(19) United States
(12) Patent Application Publication
(10) Pub. No.: US 2025/0135039 A1
(43) Pub. Date: May 1, 2025

(54) INHIBITOR-RESISTANT MGMT MODIFICATIONS AND MODIFICATION OF MGMT-ENCODING NUCLEIC ACIDS

(71) Applicants: Ensoma, Inc., Boston, MA (US); Fred Hutchinson Cancer Center, Seattle, WA (US)

(72) Inventors: Robert Thomas Peters, Needham, MA (US); Hans-Peter Kiem, Seattle, WA (US); Ashvin Reddy Bashyam, Cambridge, MA (US); Anthony Leo Forget, Charlton, MA (US); Olivier Humbert, Seattle, WA (US)

(21) Appl. No.: 18/836,708

(22) PCT Filed: Feb. 7, 2023
(86) PCT No.: PCT/US23/12540

§ 371 Date: Aug. 7, 2024

Related U.S. Application Data
(60) Provisional application No. 63/307,426, filed on Feb. 7, 2022.
(60) Provisional application No. 63/307,427, filed on Feb. 7, 2022.
(60) Provisional application No. 63/307,429, filed on Feb. 7, 2022.

(51) Int. Cl. A61K 48/00; A61K 38/45; C12N 9/22; C12N 15/11

(52) U.S. Cl. CPC A61K 48/0058; A61K 38/45; C12N 9/22; C12N 15/11; C12N 2310/20

(57) ABSTRACT

The present disclosure includes MGMT modifications that cause MGMT resistance to MGMT inhibitors. The present disclosure also includes in vivo, in vitro, and ex vivo modification of MGMT-encoding nucleic acids to encode and express an MGMT variant that is resistant to MGMT inhibitors. Inhibitor-resistant MGMT modifications and modification of an MGMT-encoding nucleic can occur or be used in conjunction with a therapeutic modification, e.g., in a single cell. Expression of inhibitor-resistant MGMT can selectively protect modified cells from a selection regimen, such as a selection regimen including an MGMT inhibitor and an alkylating agent. Accordingly, in vivo, in vitro, and ex vivo modification of MGMT-encoding nucleic acids can be used in gene therapy to select for modified cells.

### CROSS-REFERENCE TO RELATED APPLICATIONS

The present application claims the benefit to U.S. Provisional Application No. 63/307,426 filed on Feb. 7, 2022, U.S. Provisional Application No. 63/307,427 filed on Feb. 7, 2022, and U.S. Provisional Application No. 63/307,429 filed on Feb. 7, 2022, the contents of each of which are hereby incorporated herein in their entireties.

### BACKGROUND

Many medical conditions are caused by genetic mutation and/or are treatable, at least in part, by gene therapy. Some conditions are particularly treatable by modification of hematopoietic stem cells (HSCs). Compositions and methods for gene therapy, including HSC gene therapy, are therefore needed.

### SUMMARY

Gene therapy typically modifies some, but not all, cells of a population of target cells. One approach to increasing the frequency of modified cells includes delivering to the modified cells a gene that provides a selective advantage under a selection condition such as the presence of a selection agent. The present disclosure provides, among other things, methods and compositions that include various modifications providing a selective advantage to modified cells, e.g., by in vivo, in vitro, or ex vivo modification of endogenous nucleic acids of target cells. In particular, the present disclosure includes methods and compositions relating to various modifications disclosed herein for in vivo, in vitro, and/or ex vivo modification of endogenous MGMT-encoding nucleic acids, thereby providing a selective advantage to modified cells. The present disclosure further includes that in vivo, in vitro, and or ex vivo modification of endogenous MGMT-encoding nucleic acids to include or encode a modification as provide herein can be combined with delivery of a gene therapy payload such as a therapeutic payload.

In at least one aspect, the present disclosure provides a modified O(6)-methylguanine-DNA-methyltransferase (MGMT) polypeptide, where the modified MGMT polypeptide is resistant to O⁶-benzylguanine (O⁶BG), where the modified MGMT polypeptide includes at least one mutation selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

In at least one aspect, the present disclosure provides a nucleic acid encoding a modified O(6)-methylguanine-DNA-methyltransferase (MGMT) polypeptide, where the modified MGMT polypeptide is resistant to O⁶-benzylguanine (O⁶BG), where the modified MGMT polypeptide includes at least one mutation selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

In at least one aspect, the present disclosure provides a method including contacting an endogenous O(6)-methylguanine-DNA-methyltransferase (MGMT)-encoding nucleic acid of one or more cells of a mammalian subject with an editing enzyme to produce a modified MGMT-encoding nucleic acid, where the contacting occurs in vivo, in vitro, or ex vivo and the modified MGMT-encoding nucleic acid encodes MGMT^{P140K}. In some embodiments, the contacting occurs in vivo.

In at least one aspect, the present disclosure provides a method including contacting an endogenous O(6)-methylguanine-DNA-methyltransferase (MGMT)-encoding nucleic acid of one or more cells of a mammalian subject with an editing enzyme to produce a modified MGMT-encoding nucleic acid, where the contacting occurs in vivo, in vitro, or ex vivo and the modified MGMT-encoding nucleic acid encodes an O⁶-benzylguanine (O⁶BG)-resistant MGMT polypeptide, where the O⁶G-resistant MGMT polypeptide is not MGMT^{P140K} and/or does not include a lysine (K) at position 140 corresponding to SEQ ID NO: 1. In various embodiments, the O⁶BG-resistant MGMT polypeptide includes one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK. In various embodiments, the O⁶BG-resistant MGMT polypeptide includes one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P. In at least one aspect, the present disclosure provides a method including contacting an endogenous O(6)-methylguanine-DNA-methyltransferase (MGMT)-encoding nucleic acid of one or more cells of a mammalian subject with an editing enzyme to produce a modified MGMT-encoding nucleic acid, where the contacting occurs in vivo, in vitro, or ex vivo and the modified MGMT-encoding nucleic acid encodes an O⁶-benzylguanine (O⁶BG)-resistant MGMT polypeptide, where the O⁶BG-resistant MGMT polypeptide includes one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK. In at least one aspect, the present disclosure provides a method including contacting an endogenous O(6)-methylguanine-DNA-methyltransferase (MGMT)-encoding nucleic acid of one or more cells of a mammalian subject with an editing enzyme to produce a modified MGMT-encoding nucleic acid, where the contacting occurs in vivo, in vitro, or ex vivo and the modified MGMT-encoding nucleic acid encodes an O⁶-benzylguanine (O⁶BG)-resistant MGMT polypeptide, where the O⁶BG-resistant MGMT polypeptide includes one or more amino acid mutations selected from L33F, L33K, L33P, L33R, L33W, L33Y, M134F, M134V, M134W, M134Y, R135G, R135K, R135L, R135T, N137D, N137F. N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P. In various embodiments, each of the one or more amino acid mutations is encoded in the modified MGMT-encoding nucleic acid by a corresponding nucleic acid mutation selected from Table 1 and/or Table 2. In various embodiments, the contacting occurs in vivo.

In at least one aspect, the present disclosure provides use of an editing enzyme for modification of endogenous an endogenous O(6)-methylguanine-DNA-methyltransferase (MGMT)-encoding nucleic acid of one or more cells of a mammalian subject to produce a modified MGMT-encoding nucleic acid, where the use includes contacting the endogenous MGMT-encoding nucleic acid with the editing enzyme in vivo, in vitro, or ex vivo and the modified MGMT-encoding nucleic acid encodes MGMT^{P140K}. In some embodiments, the contacting occurs in vivo.

In at least one aspect, the present disclosure provides use of an editing enzyme for modification of endogenous an endogenous O(6)-methylguanine-DNA-methyltransferase (MGMT)-encoding nucleic acid of one or more cells of a mammalian subject to produce a modified MGMT-encoding nucleic acid, where the use includes contacting the endogenous MGMT-encoding nucleic acid with the editing enzyme in vivo, in vitro, or ex vivo and the modified MGMT-encoding nucleic acid encodes an O⁶-benzylguanine (O⁶BG)-resistant MGMT polypeptide, where the O⁶BG-resistant MGMT polypeptide is not MGMT^{P140K} and/or does not include a lysine (K) at position 140 corresponding to SEQ ID NO: 1. In various embodiments, the O⁶BG-resistant MGMT polypeptide includes one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK. In various embodiments, the O⁶BG-resistant MGMT polypeptide includes one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P. In at least one aspect, the present disclosure provides use of an editing enzyme for modification of endogenous an endogenous O(6)-methylguanine-DNA-methyltransferase (MGMT)-encoding nucleic acid of one or more cells of a mammalian subject to produce a modified MGMT-encoding nucleic acid, where the use includes contacting the endogenous MGMT-encoding nucleic acid with the editing enzyme in vivo, in vitro, or ex vivo and the modified MGMT-encoding nucleic acid encodes an O⁶-benzylguanine (O⁶BG)-resistant MGMT polypeptide, where the O⁶BG-resistant MGMT polypeptide includes one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140) PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK. In at least one aspect, the present disclosure provides use of an editing enzyme for modification of endogenous an endogenous O(6)-methylguanine-DNA-methyltransferase (MGMT)-encoding nucleic acid of one or more cells of a mammalian subject to produce a modified MGMT-encoding nucleic acid, where the use includes contacting the endogenous MGMT-encoding nucleic acid with the editing enzyme in vivo, in vitro, or ex vivo and the modified MGMT-encoding nucleic acid encodes an O⁶-benzylguanine (O⁶BG)-resistant MGMT polypeptide, where the O⁶BG-resistant MGMT polypeptide includes one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P. In various embodiments, each of the one or more amino acid mutations is encoded in the modified MGMT-encoding nucleic acid by a corresponding nucleic acid mutation selected from Table 1 and/or Table 2. In various embodiments, the contacting occurs in vivo.

In various embodiments, a method or use of the present disclosure includes administering to the mammalian subject a nucleic acid encoding the editing enzyme. In various embodiments, the nucleic acid encoding the editing enzyme further encodes a guide RNA that directs editing of the endogenous MGMT-encoding nucleic acid by the editing enzyme. In various embodiments, the nucleic acid encoding the editing enzyme is administered parenterally. In various embodiments, the nucleic acid encoding the editing enzyme is administered by injection. In various embodiments, the nucleic acid encoding the editing enzyme is administered intravenously.

In various embodiments, a method or use of the present disclosure includes mobilization of hematopoietic stem cells of the subject prior to administration of the nucleic acid. In various embodiments, a method or use of the present disclosure includes administering one or more immunosuppression agents to the subject, optionally where the administration of the one or more immunosuppression agents is prior to the administration of the nucleic acid. In various embodiments, a method or use of the present disclosure includes administering one or more MGMT inhibitors to the subject after the nucleic acid has been administered. In various embodiments, the one or more MGMT inhibitors includes O⁶BG or an analog or derivative thereof, and/or where the one or more MGMT inhibitors includes Lomeguatrib. In various embodiments, a method or use of the present disclosure includes administering one or more alkylating agents to the subject after the nucleic acid has been administered. In various embodiments, the one or more alkylating agents include 1,3-bis(2-chloroethyl)-1-nitrosourea (BCNU) or temozolomide.

In various embodiments, the modified MGMT-encoding nucleic acid confers a selective advantage to, and/or permits selection of, cells including the modified MGMT-encoding nucleic acid. In various embodiments, a method or use of the present disclosure includes selecting for cells including the modified MGMT-encoding nucleic acid.

In at least one aspect, the present disclosure provides a nucleic acid encoding an editing enzyme and optionally further encoding a guide RNA, where the editing enzyme, upon contact with an O(6)-methylguanine-DNA-methyltransferase (MGMT)-encoding nucleic acid, produces a modified MGMT-encoding nucleic acid that encodes MGMT^{P140K} In various embodiments, the nucleic acid encoding the editing enzyme encodes a guide RNA that directs editing of the endogenous MGMT-encoding nucleic acid by the editing enzyme.

In at least one aspect, the present disclosure provides a nucleic acid encoding an editing enzyme and optionally further encoding a guide RNA, where the editing enzyme, upon contact with an O(6)-methylguanine-DNA-methyltransferase (MGMT)-encoding nucleic acid, produces a modified MGMT-encoding nucleic acid that encodes an O⁶-benzylguanine (O⁶BG)-resistant MGMT polypeptide, where the O⁶BG-resistant MGMT polypeptide is not MGMT^{P140K} and/or does not include a lysine (K) at position 140 corresponding to SEQ ID NO: 1. In various embodiments, the O⁶BG-resistant MGMT polypeptide includes one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK. In various embodiments, the O⁶BG-resistant MGMT polypeptide includes one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P. In at least one aspect, the present disclosure provides a nucleic acid encoding an editing enzyme and optionally further encoding a guide RNA, where the editing enzyme, upon contact with an O(6)-methylguanine-DNA-methyltransferase (MGMT)-encoding nucleic acid, produces a modified MGMT-encoding nucleic acid that encodes an O⁶-benzylguanine (O⁶BG)-resistant MGMT polypeptide, where the O⁶BG-resistant MGMT polypeptide includes one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140) NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK. In at least one aspect, the present disclosure provides a nucleic acid encoding an editing enzyme and optionally further encoding a guide RNA, where the editing enzyme, upon contact with an O(6)-methylguanine-DNA-methyltransferase (MGMT)-encoding nucleic acid, produces a modified MGMT-encoding nucleic acid that encodes an O⁶-benzylguanine (O⁶BG)-resistant MGMT polypeptide, where the O⁶G-resistant MGMT polypeptide includes one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P. In various embodiments, each of the one or more amino acid mutations is encoded in the modified MGMT-encoding nucleic acid by a corresponding nucleic acid mutation selected from Table 1 and/or Table 2. In various embodiments, the nucleic acid encoding the editing enzyme encodes a guide RNA that directs editing of the endogenous MGMT-encoding nucleic acid by the editing enzyme.

In at least one aspect, the present disclosure provides a pharmaceutical composition including the nucleic acid encoding an editing enzyme of the present disclosure. In various embodiments, the pharmaceutical composition is formulated for administration to a mammalian subject, optionally where the mammalian subject is a human subject. In various embodiments, the pharmaceutical composition is formulated for parenteral administration. In various embodiments, the pharmaceutical composition is formulated for injection. In various embodiments, the pharmaceutical composition is formulated for intravenous injection.

In at least one aspect, the present disclosure provides a kit that includes a nucleic acid encoding an editing enzyme of the present disclosure and/or the pharmaceutical composition of the present disclosure. In various embodiments, the kit includes one or more MGMT inhibitors. In various embodiments, the one or more MGMT inhibitors includes O⁶BG or an analog or derivative thereof, and/or where the one or more MGMT inhibitors includes Lomeguatrib. In various embodiments, the kit includes one or more alkylating agents. In various embodiments, the one or more alkylating agents include 1,3-bis(2-chloroethyl)-1-nitrosourea (BCNU) or temozolomide. In various embodiments, the kit includes one or more mobilization agents. In various embodiments, the kit includes one or more immunosuppression agents. In various embodiments, the kit includes instructions for selection for cells including modified MGMT-encoding nucleic acids.

In various embodiments, a method, use, nucleic acid, pharmaceutical composition, or kit of the present disclosure includes an editing enzyme that is a base editing enzyme that deaminates a nucleobase in the endogenous MGMT-encoding nucleic acid. In various embodiments, the base editing enzyme includes a DNA binding domain and a deaminase domain. In various embodiments, the DNA binding domain and deaminase domain are fused. In various embodiments, the DNA binding domain is a zinc finger domain. In various embodiments, the DNA binding domain is a TALEN domain. In various embodiments, the DNA binding domain is an RNA guided DNA binding domain. In various embodiments, the RNA guided DNA binding domain is a modified Cas9 variant or a modified Cas12a variant. In various embodiments, the RNA guided DNA binding domain is a catalytically impaired nuclease domain. In various embodiments, the RNA guided DNA binding domain is a nickase variant. In various embodiments, the deaminase domain is a cytidine deaminase domain. In various embodiments, the deaminase domain is an adenosine deaminase domain.

In various embodiments, a method, use, nucleic acid, pharmaceutical composition, or kit of the present disclosure includes an editing enzyme that is a prime editing enzyme that includes a DNA binding domain and a reverse transcriptase domain.

In various embodiments, the DNA binding domain is an RNA guided DNA binding domain. In various embodiments, the RNA guided DNA binding domain and reverse transcriptase domain are fused. In various embodiments, the RNA guided DNA binding domain is a modified Cas9 variant or a modified Cas12a variant. In various embodiments, the RNA guided DNA binding domain is a catalytically impaired nuclease domain. In various embodiments, the RNA guided DNA binding domain is a nickase variant. In various embodiments, the reverse transcriptase domain is an MLV reverse transcriptase domain.

In various embodiments, a method, use, nucleic acid, pharmaceutical composition, or kit of the present disclosure includes an editing enzyme that is an RNA editing enzyme that deaminates a nucleobase in mRNA transcripts produced from the endogenous MGMT gene to produce a modified MGMT mRNA transcript.

In various embodiments, a method, use, nucleic acid, pharmaceutical composition, or kit of the present disclosure includes a nucleic acid encoding an editing enzyme where the nucleic acid is encapsidated in a viral particle. In various embodiments, the viral particle is a recombinant adenovirus. In various embodiments, the recombinant adenovirus is a recombinant Ad35 virus. In various embodiments, the recombinant adenovirus is a recombinant Ad5 virus. In various embodiments, the recombinant adenovirus is an Ad3, Ad7, Ad11, Ad14, Ad16, Ad21, Ad34, Ad37, or Ad50 virus. In various embodiments, the recombinant adenovirus is a chimeric adenovirus. In various embodiments, the chimeric adenovirus is an Ad5/35 virus (e.g., an Ad5/35++ virus).

In various embodiments of a method, use, nucleic acid, pharmaceutical composition, or kit of the present disclosure a nucleic acid encoding the editing enzyme is encapsulated in a lipid nanoparticle. In various embodiments of a method, use, nucleic acid, pharmaceutical composition, or kit of the present disclosure a nucleic acid encoding an editing enzyme is encapsulated in a liposome.

In various embodiments, a method, use, nucleic acid, pharmaceutical composition, or kit of the present disclosure includes a nucleic acid encoding an editing enzyme where the nucleic acid further includes a therapeutic payload. In various embodiments, the therapeutic payload is a non-integrating payload. In various embodiments, the therapeutic payload is an integrating payload, optionally where the integrating payload does not encode the editing enzyme. In various embodiments, the therapeutic payload comprises a nucleic acid encoding a globin protein, where the globin protein comprises a y-globin, a β-globin, and/or an a-globin. In various embodiments, the therapeutic payload comprises a nucleic acid encoding a chimeric antigen receptor (CAR), engineered T-cell receptor (TCR), checkpoint inhibitor, and/or therapeutic antibody.

In various embodiments of a method, use, nucleic acid, pharmaceutical composition, or kit of the present disclosure, the endogenous MGMT-encoding nucleic acid is an MGMT gene in the genomes of the one or more cells.

In various embodiments of a method, use, nucleic acid, pharmaceutical composition, or kit of the present disclosure, the endogenous MGMT-encoding nucleic acid is an MGMT mRNA transcript expressed from an MGMT gene of a genome of the one or more cells.

In various embodiments of a method, use, nucleic acid, pharmaceutical composition, or kit of the present disclosure, the cells are hematopoietic cells.

In various embodiments of a method, use, nucleic acid, pharmaceutical composition, or kit of the present disclosure, the cells are hematopoietic stem cells.

In at least one aspect, the present disclosure provides a method including contacting an endogenous O(6)-methylguanine-DNA-methyltransferase (MGMT)-encoding nucleic acid of one or more cells of a mammalian subject with an editing enzyme to produce a modified MGMT-encoding nucleic acid, where the contacting occurs in vivo, in vitro, or ex vivo and the modified MGMT-encoding nucleic acid encodes MGMT^{P140K}. In some embodiments, the contacting occurs in vivo.

In at least one aspect, the present disclosure provides use of an editing enzyme for modification of endogenous an endogenous O(6)-methylguanine-DNA-methyltransferase (MGMT)-encoding nucleic acid of one or more cells of a mammalian subject to produce a modified MGMT-encoding nucleic acid, where the use includes contacting the endogenous MGMT-encoding nucleic acid with the editing enzyme in vivo, in vitro, or ex vivo and the modified MGMT-encoding nucleic acid encodes MGMT^{P140K}. In some embodiments, the contacting occurs in vivo.

In various embodiments, a method or use of the present disclosure includes administering to the mammalian subject a nucleic acid encoding the editing enzyme. In various embodiments, the nucleic acid encoding the editing enzyme further encodes a guide RNA that directs editing of the endogenous MGMT-encoding nucleic acid by the editing enzyme. In various embodiments, the nucleic acid encoding the editing enzyme is administered parenterally. In various embodiments, the nucleic acid encoding the editing enzyme is administered by injection. In various embodiments, the nucleic acid encoding the editing enzyme is administered intravenously.

In various embodiments, a method or use of the present disclosure includes mobilization of hematopoietic stem cells (HSCs) of the subject prior to administration of the nucleic acid. In various embodiments, a method or use of the present disclosure includes one or more immunosuppression agents to the subject, optionally where the administration of the one or more immunosuppression agents is prior to the administration of the nucleic acid. In various embodiments, a method or use of the present disclosure includes administering one or more MGMT inhibitors to the subject after the nucleic acid has been administered. In various embodiments, the one or more MGMT inhibitors includes O⁶BG or an analog or derivative thereof, and/or where the one or more MGMT inhibitors includes Lomeguatrib. In various embodiments, a method or use of the present disclosure includes administering one or more alkylating agents to the subject after the nucleic acid has been administered. In various embodiments, the one or more alkylating agents include 1,3-bis(2-chloroethyl)-1-nitrosourea (BCNU) or temozolomide.

In various embodiments, the modified MGMT-encoding nucleic acid confers a selective advantage to, and/or permits selection of, cells including the modified MGMT-encoding nucleic acid. In various embodiments, a method or use of the present disclosure includes selecting for cells including the modified MGMT-encoding nucleic acid.

In at least one aspect, the present disclosure provides a nucleic acid encoding an editing enzyme and optionally further encoding a guide RNA, where the editing enzyme, upon contact with an O(6)-methylguanine-DNA-methyltransferase (MGMT)-encoding nucleic acid, produces a modified MGMT-encoding nucleic acid that encodes MGMT^{P140K} In various embodiments, the nucleic acid encoding the editing enzyme encodes a guide RNA that directs editing of the endogenous MGMT-encoding nucleic acid by the editing enzyme.

In at least one aspect, the present disclosure provides a pharmaceutical composition including a nucleic acid encoding an editing enzyme of the present disclosure. In various embodiments, the pharmaceutical composition is formulated for administration to a mammalian subject, optionally where the mammalian subject is a human subject. In various embodiments, the pharmaceutical composition is formulated for parenteral administration. In various embodiments, the pharmaceutical composition is formulated for injection. In various embodiments, the pharmaceutical composition is formulated for intravenous injection.

In at least one aspect, the present disclosure provides a kit that includes a nucleic acid encoding an editing enzyme as disclosed herein and/or a pharmaceutical composition of the present disclosure. In various embodiments, the kit includes one or more MGMT inhibitors. In various embodiments, the one or more MGMT inhibitors includes O⁶BG or an analog or derivative thereof, and/or where the one or more MGMT inhibitors includes Lomeguatrib. In various embodiments, the kit includes one or more alkylating agents. In various embodiments, the one or more alkylating agents include 1,3-bis(2-chloroethyl)-1-nitrosourea (BCNU) or temozolomide. In various embodiments, the kit includes one or more mobilization agents. In various embodiments, the kit includes one or more immunosuppression agents. In various embodiments, the kit includes instructions for selection for cells including modified MGMT-encoding nucleic acids.

In various embodiments, a method, use, nucleic acid, pharmaceutical composition, or kit of the present disclosure includes an editing enzyme that is a base editing enzyme that deaminates a nucleobase in the endogenous MGMT-encoding nucleic acid. In various embodiments, the base editing enzyme includes a DNA binding domain and a deaminase domain. In various embodiments, the DNA binding domain and deaminase domain are fused. In various embodiments, the DNA binding domain is a zinc finger domain. In various embodiments, the DNA binding domain is a TALEN domain. In various embodiments, the DNA binding domain is an RNA guided DNA binding domain. In various embodiments, the RNA guided DNA binding domain is a modified Cas9 variant or a modified Cas12a variant. In various embodiments, the RNA guided DNA binding domain is a catalytically impaired nuclease domain. In various embodiments, RNA guided DNA binding domain is a nickase variant. In various embodiments, the deaminase domain is a cytidine deaminase domain. In various embodiments, the deaminase domain is an adenosine deaminase domain.

In various embodiments, a method, use, nucleic acid, pharmaceutical composition, or kit of the present disclosure includes an editing enzyme that is a prime editing enzyme that includes a DNA binding domain and a reverse transcriptase domain.

In various embodiments, the DNA binding domain is an RNA guided DNA binding domain. In various embodiments, the RNA guided DNA binding domain and reverse transcriptase domain are fused. In various embodiments, the RNA guided DNA binding domain is a modified Cas9 variant or a modified Cas12a variant. In various embodiments, the RNA guided DNA binding domain is a catalytically impaired nuclease domain. In various embodiments, the RNA guided DNA binding domain is a nickase variant. In various embodiments, the reverse transcriptase domain is an MLV reverse transcriptase domain.

In various embodiments, a method, use, nucleic acid, pharmaceutical composition, or kit of the present disclosure includes an editing enzyme that is an RNA editing enzyme that deaminates a nucleobase in mRNA transcripts produced from the endogenous MGMT gene to produce a modified MGMT mRNA transcript.

In various embodiments, a method, use, nucleic acid, pharmaceutical composition, or kit of the present disclosure includes a nucleic acid encoding an editing enzyme where the nucleic acid is encapsidated in a viral particle. In various embodiments, the viral particle is a recombinant adenovirus. In various embodiments, the recombinant adenovirus is a recombinant Ad35 virus. In various embodiments, the recombinant adenovirus is a recombinant Ad5 virus. In various embodiments, the recombinant adenovirus is an Ad3, Ad7, Ad11, Ad14, Ad16, Ad21, Ad34, Ad37, or Ad50 virus. In various embodiments, the recombinant adenovirus is a chimeric adenovirus. In various embodiments, the chimeric adenovirus is an Ad5/35 virus (e.g., an Ad5/35++ virus).

In various embodiments of a method, use, nucleic acid, pharmaceutical composition, or kit of the present disclosure a nucleic acid encoding an editing enzyme is encapsulated in a lipid nanoparticle. In various embodiments of a method, use, nucleic acid, pharmaceutical composition, or kit of the present disclosure a nucleic acid encoding an editing enzyme is encapsulated in a liposome.

In various embodiments, a method, use, nucleic acid, pharmaceutical composition, or kit of the present disclosure includes a nucleic acid encoding an editing enzyme where the nucleic acid further includes a therapeutic payload. In various embodiments, the therapeutic payload is a non-integrating payload. In various embodiments, the therapeutic payload is an integrating payload, optionally where the integrating payload does not encode the editing enzyme. In various embodiments, the therapeutic payload comprises a nucleic acid encoding a globin protein, where the globin protein comprises a y-globin, a B-globin, and/or an a-globin. In various embodiments, the therapeutic payload comprises a nucleic acid encoding a chimeric antigen receptor (CAR), engineered T-cell receptor (TCR), checkpoint inhibitor, and/or therapeutic antibody.

In various embodiments of a method, use, nucleic acid, pharmaceutical composition, or kit of the present disclosure, the endogenous MGMT-encoding nucleic acid is an MGMT gene in the genomes of the one or more cells.

In various embodiments of a method, use, nucleic acid, pharmaceutical composition, or kit of the present disclosure, the endogenous MGMT-encoding nucleic acid is an MGMT mRNA transcript expressed from an MGMT gene of a genome of the one or more cells.

In various embodiments of a method, use, nucleic acid, pharmaceutical composition, or kit of the present disclosure, the cells are hematopoietic cells.

In various embodiments of a method, use, nucleic acid, pharmaceutical composition, or kit of the present disclosure, the cells are hematopoietic stem cells.

### BRIEF DESCRIPTION OF THE DRAWINGS

FIGS. 1A-1D. FIG. 1A is a schematic showing fetal hemoglobin (HPFH) mutations introduced with ABE within the HBG promoter to disrupt binding of repressor proteins (ZBTB7A, BCL11A) or to introduce site for activators (KLF1, TAL1, GATA1). FIG. 1B is a graph that shows editing efficiency. FIG. 1C is a graph that shows corresponding HbF reactivation measured in erythroid-differentiated human CD34+ cells edited by ABE7.19 protein electroporation targeting the sites shown in FIG. 1A. FIG. 1D is a graph that shows editing efficiency in CD34+ HSCs edited using increasing amounts of CBE protein targeting the HPFH-113 site.

### DEFINITIONS

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Associated with: Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another (e.g., directly or via a linker, e.g., in a fusion polypeptide); in some embodiments, two or more entities that are physically associated with one another are not covalently linked to one another but are non-covalently associated, for example by means of hydrogen bonds, van der Waals interaction, hydrophobic interactions, magnetism, or a combination thereof.

Between or From: As used herein, the term “between” refers to content that falls between indicated upper and lower, or first and second, boundaries, inclusive of the boundaries. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents: indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide; in some such embodiments, a domain is characterized by a particular structural element (e.g., a particular amino acid sequence or sequence motif, a-helix character, B-sheet character, coiled-coil character, random coil character, etc.), and/or by a particular functional feature (e.g., binding activity, enzymatic activity, folding activity, signaling activity, etc.). In some embodiments, a domain is or includes a characteristic portion or characteristic sequence element. In the present disclosure, reference to a polypeptide such as an enzyme can refer to an identified enzyme or a domain thereof (e.g., a domain having an identified activity), or to a variant of either of these.

Dosing regimen: As used herein, the term “dosing regimen” can refer to a set of one or more same or different unit doses administered to a subject, typically including a plurality of unit doses administration of each of which is separated from administration of the others by a period of time. In various embodiments, one or more or all unit doses of a dosing regimen may be the same or can vary (e.g., increase over time, decrease over time, or be adjusted in accordance with the subject and/or with a medical practitioner's determination). In various embodiments, one or more or all of the periods of time between each dose may be the same or can vary (e.g., increase over time, decrease over time, or be adjusted in accordance with the subject and/or with a medical practitioner's determination). In some embodiments, a given therapeutic agent has a recommended dosing regimen, which can involve one or more doses. Typically, at least one recommended dosing regimen of a marketed drug is known to those of skill in the art. In some embodiments, a dosing regimen is correlated with a desired or beneficial outcome when administered across a relevant population (i.e., is a therapeutic dosing regimen).

Downstream and Upstream: As used herein, the term” downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a formulation necessary to result in a desired physiological change in a subject. Effective amounts are often administered for research purposes.

Endogenous: As used herein, an agent is “endogenous” if it is naturally present in a relevant context (e.g., in a cell or organism) and/or is not present in the context as the result of engineering. For example, a nucleic acid sequence can be referred to as “endogenous” to a cell if it is present in and/or expressed from a genomic coding sequence of the cell, e.g., a genomic sequence that has not been engineered, a genomic sequence present in the cell at the time of completion of cytokinesis, and/or a genomic sequence that is derived from a germline genome of a multicellular organism in which the cell is present or from which the cell was derived.

Engineered: As used herein, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “recombinant” or “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Excipient: As used herein, “excipient” refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, or the like.

Expression: As used herein, “expression” refers individually and/or cumulatively to one or more biological process that result in production from a nucleic acid sequence of an encoded agent, such as a protein. Expression specifically includes either or both of transcription and translation.

Flank: As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means. In various instances, a fragment can alternatively be referred to as a portion.

Fusion polypeptide: As used herein, the term “fusion polypeptide” generally refers to a polypeptide including at least two segments. Typically, a polypeptide containing at least two such segments is considered to be a fusion polypeptide if the two segments are moieties that (1) are not included in nature in the same peptide, and/or (2) have not previously been linked to one another in a single polypeptide, and/or (3) have been linked to one another through action of the hand of man. A fusion polypeptide can include amino acids in addition to amino acids of two segments of the fusion polypeptide, or in addition to amino acids of the at least two segments of the polypeptide. Moieties present in a fusion polypeptide can be directly covalently associated or covalently associated via a linker. Moieties present in a fusion polypeptide can be referred to as “fused”. Fusion polypeptides can also be referred to as fusion proteins.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre-and/or post-processing) or a polypeptide (pre- and/or post-modification) encoded by an RNA transcribed from the gene.

Heterologous: As used herein, an agent is “heterologous” if it is not naturally present in a relevant context and/or is only present in the context as the result of engineering. For example, a first nucleic acid sequence is “heterologous” to a second nucleic acid sequence if the first nucleic acid sequence is not operatively linked with the second nucleic acid sequence in nature and/or in a reference context. For instance, a polypeptide is “heterologous” to a regulatory sequence if it is encoded by nucleic acid sequence that is not operatively linked with the regulatory sequence in nature and/or in a reference context.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, an intended or potential host cell can be referred to as a target cell.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Nucleotide: As used herein, the term “nucleotide” refers to a structural component, or building block, of polynucleotides, e.g., of DNA and/or RNA polymers. A nucleotide includes of a base (e.g., adenine, thymine, uracil, guanine, or cytosine) and a molecule of sugar and at least one phosphate group. As used herein, a nucleotide can be a methylated nucleotide or an un-methylated nucleotide. Those of skill in the art will appreciate that nucleic acid terminology, such as, as examples, “locus” or “nucleotide” can refer to both a locus or nucleotide of a single nucleic acid molecule and/or to the cumulative population of loci or nucleotides within a plurality of nucleic acids (e.g., a plurality of nucleic acids in a sample and/or representative of a subject) that are representative of the locus or nucleotide (e.g., having the same identical nucleic acid sequence and/or nucleic acid sequence context, or having a substantially identical nucleic acid sequence and/or nucleic acid context). Those of skill in the art will appreciate that terms relating to nucleotides, nucleobases, and nucleosides are related and in some instances are used interchangeably to refer to components of nucleic acids as appropriate in a provided context. For the avoidance of doubt, the term nucleic acid as used herein can refer to one or both of a DNA molecule (e.g., a single-stranded or double-stranded DNA molecule, such as genomic DNA) and an RNA molecule (e.g., a single-stranded or double-stranded RNA molecule) such as an mRNA transcript.

Operably linked: As used herein, “operably linked” or “operatively linked” refers to the association of at least a first element and a second element such that the component elements are in a relationship permitting them to function in their intended manner. For example, a nucleic acid regulatory sequence is “operably linked” to a nucleic acid coding sequence if the regulatory sequence and coding sequence are associated in a manner that permits control of expression of the coding sequence by the regulatory sequence. In some embodiments, an “operably linked” regulatory sequence is directly or indirectly covalently associated with a coding sequence (e.g., in a single nucleic acid). In some embodiments, a regulatory sequence controls expression of a coding sequence in trans and inclusion of the regulatory sequence in the same nucleic acid as the coding sequence is not a requirement of operable linkage.

Pharmaceutically acceptable: As used herein, the term “pharmaceutically acceptable,” as applied to one or more, or all, component(s) for formulation of a composition as disclosed herein, means that each component must be compatible with the other ingredients of the composition and not deleterious to the recipient thereof.

Pharmaceutically acceptable carrier: As used herein, the term “pharmaceutically acceptable carrier” refers to a pharmaceutically-acceptable material, composition, or vehicle, such as a liquid or solid filler, diluent, excipient, or solvent encapsulating material, that facilitates formulation of an agent (e.g., a pharmaceutical agent), modifies bioavailability of an agent, or facilitates transport of an agent from one organ or portion of a subject to another. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to a composition in which an active agent is formulated together with one or more pharmaceutically acceptable carriers.

Polypeptide: As used herein, “polypeptide” refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may be or include of natural amino acids, non-natural amino acids, or both. In some embodiments, a polypeptide may be or include only natural amino acids or only non-natural amino acids. In some embodiments, a polypeptide can include D-amino acids, L-amino acids, or both. In some embodiments, a polypeptide may include only L-amino acids. In some embodiments, a polypeptide may include one or more pendant groups or other modifications, e.g., one or more amino acid side chains, e.g., at the polypeptide's N-terminus, at the polypeptide's C-terminus, at non-terminal amino acids, or at any combination thereof. In some embodiments, such pendant groups or modifications may be selected from acetylation, amidation, lipidation, methylation, phosphorylation, glycosylation, glycation, sulfation, mannosylation, nitrosylation, acylation, palmitoylation, prenylation, pegylation, etc., including combinations thereof. In some embodiments, a polypeptide may be cyclic, and/or may include a cyclic portion.

In some embodiments, the term “polypeptide” may be appended to a name of a reference polypeptide, activity, or structure to indicate a class of polypeptides that share a relevant activity or structure. For such classes, the present specification provides and/or those skilled in the art will be aware of exemplary polypeptides within the class whose amino acid sequences and/or functions are known. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class. For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that can in some embodiments be or include a characteristic sequence element) that shows very high sequence identity, often greater than 90% or even 95%, 96%, 97%, 98%, or 99%. Such a conserved region usually encompasses at least 3-4 and in some instances up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide can be or include a fragment of a parent polypeptide. In some embodiments, a useful polypeptide may be or include a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

For the avoidance of doubt, where a polypeptide (or a nucleic acid sequence encoding a polypeptide) is referred to by a particular name, which particular name is in some instances associated with one or more particular reference sequences, those of skill in the art will appreciate that the particular name can include and be used to refer to both the particular reference sequences and to variants thereof (e.g., variants having at least 80%, 8%, 90%, 91%, 92%, 93%, 94%, 95%, 96%, 97%, 98%, 99%, 99.5%, or 99.9% identity to one or more of the reference sequences).

Promoter: As used herein, a “promoter” or “promoter sequence” can be a DNA regulatory region that directly or indirectly (e.g., through promoter-bound proteins or substances) participates in initiation and/or processivity of transcription of a coding sequence. A promoter may, under suitable conditions, initiate transcription of a coding sequence upon binding of one or more transcription factors and/or regulatory moieties with the promoter. A promoter that participates in initiation of transcription of a coding sequence can be “operably linked” to the coding sequence. In certain instances, a promoter can be or include a DNA regulatory region that extends from a transcription initiation site (at its 3′ terminus) to an upstream (5′ direction) position such that the sequence so designated includes one or both of a minimum number of bases or elements necessary to initiate a transcription event. A promoter may be, include, or be operably associated with or operably linked to, expression control sequences such as enhancer and repressor sequences. In some embodiments, a promoter may be inducible. In some embodiments, a promoter may be a constitutive promoter. In some embodiments, a conditional (e.g., inducible) promoter may be unidirectional or bi-directional. A promoter may be or include a sequence identical to a sequence known to occur in the genome of particular species. In some embodiments, a promoter can be or include a hybrid promoter, in which a sequence containing a transcriptional regulatory region can be obtained from one source and a sequence containing a transcription initiation region can be obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, NY, 1989).

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represents measure under comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof.

Regulatory sequence: As used herein in the context of expression of a nucleic acid coding sequence, a regulatory sequence is a nucleic acid sequence that controls expression of a coding sequence. In some embodiments, a regulatory sequence can control or impact one or more aspects of gene expression (e.g., cell-type-specific expression, inducible expression, etc.).

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a human subject can be interchangeably referred to as a “patient” or “individual.”

Therapeutic agent: As used herein, the term “therapeutic agent” refers to any agent that elicits a desired pharmacological effect when administered to a subject. In some embodiments, an agent is considered to be a therapeutic agent if it demonstrates a statistically significant effect across an appropriate population. In some embodiments, the appropriate population can be a population of model organisms or a human population. In some embodiments, an appropriate population can be defined by various criteria, such as a certain age group, gender, genetic background, preexisting clinical conditions, etc. In some embodiments, a therapeutic agent is a substance that can be used for treatment of a disease, disorder, or condition. In some embodiments, a therapeutic agent is an agent that has been or is required to be approved by a government agency before it can be marketed for administration to humans. In some embodiments, a therapeutic agent is an agent for which a medical prescription is required for administration to humans.

Therapeutically effective amount: As used herein, “therapeutically effective amount” refers to an amount that produces the desired effect for which it is administered. In some embodiments, the term refers to an amount that is sufficient, when administered to a population suffering from or susceptible to a disease, disorder, and/or condition in accordance with a therapeutic dosing regimen, to treat the disease, disorder, and/or condition. In some embodiments, a therapeutically effective amount is one that reduces the incidence and/or severity of, and/or delays onset of, one or more symptoms of the disease, disorder, and/or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine, etc.). Those of ordinary skill in the art will appreciate that, in some embodiments, a therapeutically effective amount of a particular agent or therapy may be formulated and/or administered in a single dose. In some embodiments, a therapeutically effective agent may be formulated and/or administered in a plurality of doses, for example, as part of a dosing regimen.

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

Unit dose: As used herein, the term “unit dose” refers to an amount administered as a single dose and/or in a physically discrete unit of a pharmaceutical composition. In many embodiments, a unit dose contains a predetermined quantity of an active agent, for instance a predetermined viral titer (the number of viruses, virions, or viral particles in a given volume). In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose can be, for example, a volume of liquid (e.g., an acceptable carrier) containing a predetermined quantity of one or more therapeutic moieties, a predetermined amount of one or more therapeutic moieties in solid form, a sustained release formulation or drug delivery device containing a predetermined amount of one or more therapeutic moieties, etc. It will be appreciated that a unit dose can be present in a formulation that includes any of a variety of components in addition to the therapeutic moiety(s). For example, acceptable carriers (e.g., pharmaceutically acceptable carriers), diluents, stabilizers, buffers, preservatives, etc., can be included. It will be appreciated by those skilled in the art, in many embodiments, a total appropriate daily dosage of a particular therapeutic agent can include a portion, or a plurality, of unit doses, and can be decided, for example, by a medical practitioner within the scope of sound medical judgment. In some embodiments, the specific effective dose level for any particular subject or organism can depend upon a variety of factors including the disorder being treated and the severity of the disorder; activity of specific active compound employed; specific composition employed; age, body weight, general health, sex, and diet of the subject; time of administration, and rate of excretion of the specific active compound employed; duration of the treatment; drugs and/or additional therapies used in combination or coincidental with specific compound(s) employed, and like factors well known in the medical arts.

Variant: As used herein, the term “variant” refers to an entity that shows significant structural identity with a reference entity but differs structurally from the reference entity in the presence, absence, or level of one or more chemical moieties as compared with the reference entity. In some embodiments, a variant also differs functionally from its reference entity. In various embodiments, a variant can be referred to as a “modified” form of a reference entity. In general, whether a particular entity is properly considered to be a “variant” of a reference entity is based on its degree of structural identity with the reference entity. A variant can be a molecule comparable, but not identical to, a reference. For example, a variant nucleic acid can differ from a reference nucleic acid at one or more differences in nucleotide sequence. In some embodiments, a variant nucleic acid shows an overall sequence identity with a reference nucleic acid that is at least 85%, 86%, 87%, 88%, 89%, 90%, 91%, 92%, 93%, 94%, 95%, 96%, 97%, or 99%. In many embodiments, a nucleic acid of interest is considered to be a “variant” of a reference nucleic acid if the nucleic acid of interest has a sequence that is identical to that of the reference but for a small number of sequence alterations at particular positions. In some embodiments, a variant has 10, 9, 8, 7, 6, 5, 4, 3, 2, or 1 substituted residue(s) as compared with a reference. In some embodiments, a variant has not more than 5, 4, 3, 2, or 1 residue additions, substitutions, or deletions as compared with the reference. In various embodiments, the number of additions, substitutions, or deletions is fewer than about 25, about 20, about 19, about 18, about 17, about 16, about 15, about 14, about 13, about 10, about 9, about 8, about 7, about 6, and commonly are fewer than about 5, about 4, about 3, or about 2 residues.

### DETAILED DESCRIPTION

The present disclosure includes, among other things, the identification of MGMT polypeptide sequences that are resistant to MGMT inhibitors, and further includes the recognition that in vivo, in vitro, and or ex vivo modification of endogenous O(6)-methylguanine-DNA-methyltransferase (MGMT) nucleic acids is useful in gene therapy. MGMT functions, at least in part, as a DNA repair enzyme that protects cells from alkylating agents. However, the protective functions of wild type MGMT are antagonized by MGMT inhibitors (e.g., O⁶-benzylguanine (O⁶BG), Lomeguatrib [6-(4-bromo-2-thienyl) methoxy] purin-2-amine], and others provided herein), rendering cells vulnerable to elimination by alkylating agents. The present disclosure includes in vivo, in vitro, and or ex vivo modification of MGMT-encoding nucleic acids to encode MGMT that is resistant to MGMT inhibitors (“inhibitor-resistant MGMT” or “inhibitor-resistant MGMT polypeptide”). In various embodiments, cells that encode and/or express inhibitor-resistant MGMT (“MGMT-modified cells” or “modified cells”) are less likely to be eliminated by a selection regimen that includes one or more MGMT inhibitors and optionally one or more alkylating agents. In various embodiments, elimination of cells refers to causing the death, cessation of growth, cessation of proliferation, and/or cessation of one or more biological functions of a cell, e.g., as understood by those of skill in the art to result from contact of a cell with a particular agent or regimen such as a selection regimen including one or more MGMT inhibitors and optionally one or more alkylating agents. Accordingly, administration of a selection regimen including one or more MGMT inhibitors and optionally one or more alkylating agents can positively select modified cells (e.g., MGMT-modified cells). This approach can be used in gene therapy, e.g., to increase the prevalence of in vivo, in vitro, and or ex vivo modified cells in a gene therapy subject. In various embodiments, in vivo, in vitro, and or ex vivo modification of MGMT is correlated with a further modification, e.g., a therapeutic modification, such that selection of modified cells increases the prevalence of cells including a therapeutic modification. In various embodiments, cells that are modified and/or targeted for modification are therapeutic cells. As disclosed herein, therapeutic cells can include any cells that express MGMT and/or are therapeutic at least in that they cause, elicit, or contribute to a desired pharmacological and/or physiological effect. In various embodiments, therapeutic cells are HSCs of a subject.

The present disclosure includes the recognition that various advantages are associated with in vivo, in vitro, and or ex vivo modification of endogenous MGMT-encoding nucleic acids. For example, expression of inhibitor-resistant MGMT from in vivo, in vitro, and or ex vivo modified endogenous MGMT-encoding nucleic acids can be tuned to endogenous expression levels (e.g., in that the level of expression of inhibitor-resistant MGMT polypeptides in modified cells is at most about the level of expression of endogenous MGMT in reference cells). Without wishing to be bound by any particular scientific theory, this is at least in part because expression of inhibitor-resistant MGMT from in vivo, in vitro, and or ex vivo modified endogenous MGMT-encoding nucleic acids is controlled by endogenous regulatory sequences. By contrast, transduction of cells to express a transgenic MGMT selectable marker can result in the insertion of multiple copies of the transgene, and/or can include a heterologous regulatory sequence (e.g., a heterologous promoter that causes expression of the selectable marker at a higher level than endogenous MGMT in target and/or reference cells), and this excessive expression of MGMT, including inhibitor-resistant MGMT variants, can be deleterious and has been observed to result in a growth defect. Without wishing to be bound by any particular scientific theory, deleterious effects of inhibitor-resistant MGMT overexpression may result from increased affinity for DNA as compared to reference MGMT and/or non-specific binding of chromatin. Moreover, unlike systems in which a heterologous transgene encoding inhibitor-resistant MGMT is inserted into a host cell genome, in vivo, in vitro, and or ex vivo modification of endogenous MGMT-encoding nucleic acids does not require potentially harmful insertion of an MGMT transgene. Modification of endogenous MGMT-encoding nucleic acids to generate a selectable marker, and particularly application of such an approach in vivo to generate a selectable marker in cells of a subject, runs contrary to decades of standard laboratory practices establishing heterologous transgenes as the default for introduction of a neo-functional selectable marker (i.e., a selectable marker that provides to cells a functional polypeptide that confers upon cells a biological or detectable activity or characteristic that enables selection, such as inhibitor resistance).

In particular embodiments, cells modified to encode an inhibitor-resistant MGMT are also genetically modified for an additional therapeutic purpose. In various embodiments, genetic modification for an additional therapeutic purpose can include delivery of a nucleic acid encoding a transgene and/or editing of an endogenous target nucleic acid, either of both of which can provide a therapeutic nucleic acid to a target cell. The genetic modification for the additional therapeutic purpose can provide a therapeutic nucleic acid that encodes a protein, e.g., to treat a disease, disorder, or condition. In certain embodiments, genetic modification for the additional therapeutic purpose can provide a therapeutic nucleic acid that encodes a chimeric antigen receptor (CAR), engineered T-cell receptor (TCR), checkpoint inhibitor, or therapeutic antibody. In certain embodiments, genetic modification for the additional therapeutic purpose can provide (e.g. deliver an editing system that causes) a modification of an endogenous sequence that increases expression of an endogenous globin gene.

The present disclosure further includes the recognition that methods and compositions disclosed herein allow for selection of modified cells in vivo, in vitro, and or ex vivo using pharmaceutically acceptable and/or low dosages of MGMT inhibitor, and/or pharmaceutically acceptable and/or low dosages of alkylating agent. Methods and compositions disclosed herein can increase the number of modified cells and/or prevalence or ratio of modified cells as compared to non-modified cells relative to a reference, e.g., within a particular subject, tissue, and/or cell population (e.g., a population of cells of a particular cell type, such as HSCs). In various embodiments, increasing the number of modified cells and/or prevalence or ratio of modified cells as compared to non-modified cells relative to a reference can improve efficacy of gene therapy (e.g., improve the treatment of a disease, disorder, or condition) as compared to a reference. In various embodiments, a reference is a subject, cell, or system, or population receiving the same or similar therapy except in that the reference is not administered a selection regimen. In various embodiments, a reference is the same subject, cell, or system prior to administration of a selection regimen. The present disclosure includes the recognition that, using methods and compositions provided herein, a gene therapy that initially (e.g., within 1 day, 2 days, 3 days, 4 days, 5 days, 6 days, 1 week, 2 weeks, 4 weeks, 8 weeks, or 16 weeks of administration and/or prior to administration of a selection regimen) modifies (e.g., delivers a therapeutic payload to, expresses a therapeutic payload in, and/or integrates a therapeutic payload into genomes of) a small number of cells (e.g., a number or percentage of cells insufficient to treat, substantially treat, clinically improve, substantially clinically improve, cure, and/or substantially cure a disease, disorder, or condition) can result in therapeutic efficacy and/or modification of a clinically significant number of cells (e.g., at least 1%, 2%, 3%, 4%, 5%, 10%, 15%, 20%, 25%, 30%, 40%, 50%, 60%, 70%, 80%, 90%, 91%, 92%, 93%, 94%, 95%, 96%, 97%, 98%, 99%, or 100% of target cells and/or cells of a particular subject, tissue, and/or cell population (e.g., a population of cells of a particular cell type, such as HSCs)). In various embodiments, modified cells are self-renewing, multilineage, and/or long-term repopulating cells such as HSCs. In various embodiments, modified cells express a therapeutic payload (e.g., heterologous gene encoding a polypeptide of interest) at a therapeutically effective level.

The present disclosure includes the recognition that selective protection of cells from selection regimens of the present disclosure can be accomplished by editing of MGMT-encoding nucleic acids, including without limitation endogenous MGMT genes encoded by cell genomes and/or endogenously expressed messenger ribonucleic acid (mRNA) molecules that encode MGMT. In various embodiments, a cell includes two endogenous copies of an MGMT gene and one or both MGMT genes are edited. In various embodiments, a cell includes one or a plurality of MGMT-encoding mRNA molecules expressed from a genomic MGMT gene and one or more of the MGMT-encoding mRNA molecules are edited. The present disclosure includes the recognition that editing of mRNA is more transient and/or reversible as compared to editing of genomic DNA. Transient modification can minimize any disruption of endogenous processes and/or fitness (e.g., where expression of an MGMT variant is associated with a fitness or competitive cost, e.g., in the absence of a selecting agent, as compared to reference cells).

### MGMT

MGMT is a DNA repair enzyme that can repair damaged guanine nucleotides by transferring the methyl at the 06 site of guanine to its cysteine residues, which can counteract the genotoxicity of alkylating agents. A reference MGMT polypeptide can have a sequence according to SEQ ID NO: 1 (below; see also GenBank Accession No. NP_002403.3). In various embodiments, an MGMT polypeptide of the present disclosure can have at least 80% sequence identity with SEQ ID NO: 1 (e.g., 80%, 85%, 90%, 91%, 92%, 93%, 94%, 95%, 96%, 97%, 98%, 99%, or 100% sequence identity with SEQ ID NO: 1). MGMT according to SEQ ID NO: 1 can be referred to as “canonical” or “wild type” MGMT. In various embodiments, numbering of amino acids of an MGMT polypeptide (e.g., wild type MGMT or variant of MGMT that is an inhibitor-resistant MGMT) can be based on numbering that corresponds to SEQ ID NO: 1. A portion of SEQ ID NO: 1 spanning from amino acid 139 to amino acid 207 (SEQ ID NO: 2) is enriched for amino acids that interact with MGMT inhibitors. In various embodiments, an MGMT polypeptide includes a fragment corresponding to SEQ ID NO: 2, where the fragment corresponding to SEQ ID NO: 2 has at least 80% sequence identity with SEQ ID NO: 2 (e.g., 80%, 85%, 90%, 91%, 92%, 93%, 94%, 95%, 96%, 97%, 98%, 99%, or 100% sequence identity with SEQ ID NO: 2), optionally wherein the remainder of the MGMT-encoding sequence has at least 80% sequence identity with SEQ ID NO: 1 (e.g., 80%, 85%, 90%, 91%, 92%, 93%, 94%, 95%, 96%, 97%, 98%, 99%, or 100% sequence identity with SEQ ID NO: 1).

(Wild Type MGMT)
SEQ ID NO: 1
MDKDCEMKRTTLDSPLGKLELSGCEQGLHEIKLLGKGTSAADAVE
VPAPAAVLGGPEPLMQCTAWLNAYFHQPEAIEEFPVPALHHPVFQ
QESFTRQVLWKLLKVVKFGEVISYQQLAALAGNPKAARAVGGAMR
GNPVPILIPCHRVVCSSGAVGNYSGGLAVKEWLLAHEGHRLGKPG
LGGSSGLAGAWLKGAGATSGSPPAGRN
(MGMT Fragment Enriched for Inhibitor Interaction)
SEQ ID NO: 2
VPILIPCHRVVCSSGAVGNYSGGLAVKEWLLAHEGHRLGKPGLGG
SSGLAGAWLKGAGATSGSPPAGRN

As those of skill in the art will appreciate, genes of the human genome that encode polypeptides can include one or more exons and one or more introns. Accordingly, genomic nucleotide sequences that encode and/or express polypeptides can include a larger number of nucleotides than would be minimally required to encode the sequence of the polypeptide. In various embodiments, wild type MGMT can be encoded by a nucleic acid sequence according to GenBank Accession No. NG_052673 (e.g., version NG_052673.1 of Accession No. NG_052673). As indicated in the GenBank Accession, exons include positions 74070-74194, 245712-245860, 297019-297158, and 304605-304814.

The combined MGMT coding sequences of NG_052673 can be presented as a single contiguous sequence encoding MGMT, as shown in SEQ ID NO: 3. In various embodiments, an MGMT coding sequence has at least 80% sequence identity with SEQ ID NO: 3 (e.g., 80%, 85%, 90%, 91%, 92%, 93%, 94%, 95%, 96%, 97%, 98%, 99%, or 100% sequence identity with SEQ ID NO: 3). The portion of SEQ ID NO: 3 that encodes SEQ ID NO: 2, and therefore corresponds to a portion of MGMT that is enriched for amino acids that interact with MGMT inhibitors, is provided in SEQ ID NO: 4. In various embodiments, an MGMT coding sequence includes a fragment that corresponds to SEQ ID NO: 4, where the fragment corresponding to SEQ ID NO: 4 has at least 80% sequence identity with SEQ ID NO: 4 (e.g., 80%, 85%, 90%, 91%, 92%, 93%, 94%, 95%, 96%, 97%, 98%, 99%, or 100% sequence identity with SEQ ID NO: 4), optionally wherein the remainder of the MGMT-encoding sequence has at least 80% sequence identity with SEQ ID NO: 3 (e.g., 80%, 85%, 90%, 91%, 92%, 93%, 94%, 95%, 96%, 97%, 98%, 99%, or 100% sequence identity with SEQ ID NO: 3). The genomic context of SEQ ID NO: 4, as found in NG_052673, is shown as SEQ ID NO: 5, the selected boundaries of which, as defined by the first and last included nucleotides, are not significant.

(wild type MGMT coding sequence)
SEQ ID NO: 3
atggacaaggattgtgaaatgaaacgcaccacactggacagccct
ttggggaagctggagctgtctggttgtgagcagggtctgcacgaa
ataaagctcctgggcaaggggacgtctgcagctgatgccgtggag
gtcccagcccccgctgcggttctcggaggtccggagcccctgatg
cagtgcacagcctggctgaatgcctatttccaccagcccgaggct
atcgaagagttccccgtgccggctcttcaccatcccgttttccag
caagagtcgttcaccagacaggtgttatggaagctgctgaaggtt
gtgaaattcggagaagtgatttcttaccagcaattagcagccctg
gcaggcaaccccaaagccgcgcgagcagtgggaggagcaatgaga
ggcaatcctgtccccatcctcatcccgtgccacagagtggtctgc
agcagcggagccgtgggcaactactccggaggactggccgtgaag
gaatggcttctggcccatgaaggccaccggttggggaagccaggc
ttgggagggagctcaggtctggcaggggcctggctcaagggagcg
ggagctacctcgggctccccgcctgctggccgaaactga
(Portion of SEQ ID NO: 3 that Encodes
SEQ ID NO: 2)
SEQ ID NO: 4
gtccccatcctcatcccgtgccacagagtggtctgcagcagcgga
gccgtgggcaactactccggaggactggccgtgaaggaatggctt
ctggcccatgaaggccaccggttggggaagccaggcttgggaggg
agctcaggtctggcaggggcctggctcaagggagcgggagctacc
tcgggctccccgcctgctggccgaaactga
(Positions 304081-305220 of NG 052673)
SEQ ID NO: 5
agtaaagcttcatttgcaaaagcaggtggtgggctgcgtgcgccc
acagccacagttggccggccatgggctggagaggcccgtgcaggt
acggtcttctctgatgcgtcctgtgatgcctgcgtggtacctggg
gagtgtttgagagcgtcacataccaccagaactcggaaacgtagt
tttcagttttgtaagcgaacgttgtatattactcaccggatgaaa
ttaagttttctcctcccaaatcgtgggcctaaagcaattctctga
ggatgccccacgtcacctcaggacactcgtccctcccccaacccg
tgctgagacccccagggactcaagggcctcaggggaggggaagtc
catgctgagacatagctgacacccacccatgccaacagcctgccc
ctggcacaggcccctgcttggtgggcacaggactcctgtcagtca
gggccttggccttgaccccaaagacctcgttgtccagatccctga
ctgacagtggctgcccccctgtcttccaggtccccatcctcatcc
cgtgccacagagtggtctgcagcagcggagccgtgggcaactact
ccggaggactggccgtgaaggaatggcttctggcccatgaaggcc
accggttggggaagccaggcttgggagggagctcaggtctggcag
gggcctggctcaagggagcgggagctacctcgggctccccgcctg
ctggccgaaactgagtatgtgcagtaggatggatgtttgagcgac
acacacgtgtaacactgcatcggatgcggggcgtggaggcaccgc
tgtattaaaggaagtggcagtgtcctgggaacaagcgtgtctgcc
ctttctgtttccatattttacagcaggatgagttcagacgcccgc
ggtcctgcacacatttgtttccttctctaacgctgcccttgctct
atttttcatgtccattaaaacaggccaagtgagtgtggaaggcct
ggctcatgttgggccacagcccaggatggggcagtctggcaccct
caggccacagacggctgccatagccgctgtccagggccagctaag
gcccatcccaggccgtccacactagaaagctggccctgccccatc
cccaccatgcctccc

Those of skill in the art will be familiar with the processes by which polypeptides encoded by genomic DNA are produced. Genomic sequences are transcribed to produce messenger ribonucleic acid molecules (mRNA) in which the coding sequence of a polypeptide is found as a single contiguous sequence. The process of transcription includes replacement of thymine nucleotides with uracil nucleotides.

The present disclosure includes an MGMT mRNA sequence according to SEQ ID NO: 6. SEQ ID NO: 7 is the portion of SEQ ID NO: 6 that encodes SEQ ID NO: 2. In various embodiments, an MGMT mRNA sequence has at least 80% sequence identity with SEQ ID NO: 6 (e.g., 80%, 85%, 90%, 91%, 92%, 93%, 94%, 95%, 96%, 97%, 98%, 99%, or 100% sequence identity with SEQ ID NO: 6). The portion of SEQ ID NO: 6 that encodes SEQ ID NO: 2, and therefore corresponds to a portion of MGMT that is enriched for amino acids that interact with MGMT inhibitors, is provided in SEQ ID NO: 7. In various embodiments, an MGMT mRNA sequence includes a fragment that corresponds to SEQ ID NO: 7, where the fragment corresponding to SEQ ID NO: 7 has at least 80% sequence identity with SEQ ID NO: 7 (e.g., 80%, 85%, 90%, 91%, 92%, 93%, 94%, 95%, 96%, 97%, 98%, 99%, or 100% sequence identity with SEQ ID NO: 7), optionally wherein the remainder of the MGMT mRNA sequence has at least 80% sequence identity with SEQ ID NO: 6 (e.g., 80%, 85%, 90%, 91%, 92%, 93%, 94%, 95%, 96%, 97%, 98%, 99%, or 100% sequence identity with SEQ ID NO: 6).

(Wild Type MGMT mRNA)
SEQ ID NO: 6
auggacaaggauugugaaaugaaacgcaccacacuggacagcccu
uuggggaagcuggagcugucugguugugagcagggucugcacgaa
auaaagcuccugggcaaggggacgucugcagcugaugccguggag
gucccagcccccgcugcgguucucggagguccggagccccugaug
cagugcacagccuggcugaaugccuauuuccaccagcccgaggcu
aucgaagaguuccccgugccggcucuucaccaucccguuuuccag
caagagucguucaccagacagguguuauggaagcugcugaagguu
gugaaauucggagaagugauuucuuaccagcaauuagcagcccug
gcaggcaaccccaaagccgcgcgagcagugggaggagcaaugaga
ggcaauccuguccccauccucaucccgugccacagaguggucugc
agcagcggagccgugggcaacuacuccggaggacuggccgugaag
gaauggcuucuggcccaugaaggccaccgguuggggaagccaggc
uugggagggagcucaggucuggcaggggccuggcucaagggagcg
ggagcuaccucgggcuccccgccugcuggccgaaacuga
(Portion of SEQ ID NO: 6 that Encodes
SEQ ID NO: 2)
SEQ ID NO: 7
guccccauccucaucccgugccacagaguggucugcagcagcgga
gccgugggcaacuacuccggaggacuggccgugaaggaauggcuu
cuggcccaugaaggccaccgguuggggaagccaggcuugggaggg
agcucaggucuggcaggggccuggcucaagggagcgggagcuacc
ucgggcuccccgccugcuggccgaaacuga

Overexpression of MGMT and/or variants thereof (e.g., expression at levels greater than those caused by endogenous regulatory sequences) can be harmful to cells. For example, it has been observed that high levels of MGMT^{P140K} expression can result in a competitive disadvantage and/or proliferation defect (see, e.g., Milsom 2008 Cancer Research, 68 (15), 6171-6180). Overexpression of MGMT^{P140K} was associated with reduced proliferation, engraftment, and therapeutic benefit. By contrast, the present disclosure appreciates that endogenous levels of MGMT are sufficient for protection of cells against clinically and/or therapeutically relevant and/or useful doses of alkylating agents. Moreover, the present disclosure appreciates that an MGMT variant (e.g., an inhibitor-resistant MGMT) expressed at endogenous levels, and in particular expressed at levels controlled and/or capped by endogenous regulatory sequences and/or mechanisms, are sufficient for protection of cells against clinically and/or therapeutically relevant and/or useful doses of selection regimens and agents thereof as disclosed herein.

### Inhibitor-Resistant MGMT

The present disclosure includes variants of MGMT that are resistant to MGMT inhibitors. The present disclosure includes sequence variants (also referred to herein as mutations) as shown in Table 1 unexpectedly identified as useful for producing inhibitor-resistant MGMT polypeptides, which sequence variants include L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

In various embodiments, variants of MGMT disclosed herein are retain some or all ability to repair DNA and/or protect cells from alkylating agents. In various embodiments, retention of ability to repair DNA and/or protect cells from alkylating agents refers to activity in the absence of inhibitor that is at least 5% (e.g., 10%, 20%, 30%, 40%, 50%, 100%, or more) of the activity of wild-type MGMT in the absence of the MGMT inhibitor, where activity can be measured by any assay known in the art including an assay as set forth in the present Examples (see, e.g., Example 1). In various embodiments, activity can refer to, e.g., alkyltransferase activity.

In some embodiments, an MGMT polypeptide that retains some or all ability to repair DNA and/or protect cells from an alkylating agent includes at least one amino acid mutation selected from L33F, L33K, L33P, L33R, L33W, L33Y, M134V, R135G, R135K, R135T, P138K, P140F, P140H, P140I, G156P, G156V, Y158T, S159F, S159I, S159W, S159Y, G160D, G160E, G160H, and G160P. In various embodiments, activity can refer to, e.g., alkyltransferase activity. In some embodiments, an MGMT polypeptide that retains some or all ability to repair DNA and/or protect cells from an alkylating agent includes at least one amino acid mutation selected from L33F, L33K, L33P, L33R, L33W, L33Y, M134F, M134V, R135G, R135K, R135L, R135T, N137D, P138K, P140F, P140H, P140I, G156I, G156P, G156V, Y158M, Y158T, S159F, S159I, S159W, S159Y, G160D, G160E, G160H, and G160P. In various embodiments, activity can refer to, e.g., alkyltransferase activity. In some embodiments, an MGMT polypeptide that retains some or all ability to repair DNA and/or protect cells from an alkylating agent includes at least one amino acid mutation selected from P140Q, P140R, G156A, Y158F, Y158H, G160A, and A170S. In some embodiments, an MGMT polypeptide that retains some or all ability to repair DNA and/or protect cells from an alkylating agent includes the amino acid mutation P140K.

In various embodiments, inhibitor-resistant MGMT polypeptides of the present disclosure are characterized in that they are resistant to inhibition by one or more MGMT inhibitors but retain some or all ability to repair DNA and/or protect cells from alkylating agents. In various embodiments, resistance to an MGMT inhibitor refers to activity in the presence of the MGMT inhibitor that is at least 10% (e.g., 20%, 30%, 40%, 50%, 100%, or more) greater than the activity of wild-type MGMT in the presence of the MGMT inhibitor, where activity can be measured by any assay known in the art including an assay as set forth in the present Examples (see, e.g., Example 1). In various embodiments, such activity in the presence of an MGMT inhibitor is normalized using the activity of the MGMT polypeptide in the absence of an MGMT inhibitor. In various embodiments, retention of ability to repair DNA and/or protect cells from alkylating agents refers to activity in the absence of inhibitor that is at least 5% (e.g., 10%, 20%, 30%, 40%, 50%, 100%, or more) of the activity of wild-type MGMT in the absence of the MGMT inhibitor, where activity can be measured by any assay known in the art including an assay as set forth in the present Examples (see, e.g., Example 1). In various embodiments, activity can refer to, e.g., alkyltransferase activity.

In various embodiments, an inhibitor-resistant MGMT polypeptide includes at least one amino acid mutation selected from M134F, R135L, N137D, P140H, P140I, G156I, G156P, G156V, Y158M, S159I, G160H, and G160P. In various embodiments, an inhibitor-resistant MGMT polypeptide includes at least one amino acid mutation selected from M134F, R135L, N137D, P140H, G156I, G156P, G156V, and Y158M. In various embodiments, an inhibitor-resistant MGMT polypeptide includes at least one amino acid mutation selected from P140H, P140I, G156P, G156V, Y158M, S159I, G160H, and G160P. In various embodiments, an inhibitor-resistant MGMT polypeptide includes at least one amino acid mutation selected from P140H, G156P, and G156V. In various embodiments, an inhibitor-resistant MGMT polypeptide includes at least one amino acid mutation selected from G156A, Y158H, and P140R. In various embodiments, an inhibitor-resistant MGMT polypeptide includes at least one amino acid mutation selected from Y158H and P140R. In various embodiments, an inhibitor-resistant MGMT polypeptide includes the amino acid mutation P140K.

In various embodiments, MGMT polypeptides of the present disclosure are characterized in that they are more sensitive to inhibition by one or more MGMT inhibitors. In various embodiments, sensitivity to an MGMT inhibitor refers to activity in the presence of the MGMT inhibitor that is at least 10% (e.g., 20%, 30%, 40%, 50%, 100%, or more) less than the activity of wild-type MGMT in the presence of the MGMT inhibitor, where activity can be measured by any assay known in the art including an assay as set forth in the present Examples (see, e.g., Example 1). In various embodiments, such activity in the presence of an MGMT inhibitor is normalized using the activity of the MGMT polypeptide in the absence of an MGMT inhibitor. In various embodiments, activity can refer to, e.g., alkyltransferase activity.

In some embodiments, an MGMT polypeptide that is more sensitive to inhibition by one or more MGMT inhibitors includes at least one amino acid mutation selected from L33F, L33P, L33W, L33Y, R135G, R135K, S159F, S159Y, and G160E. In some embodiments, an MGMT polypeptide that is more sensitive to inhibition by one or more MGMT inhibitors includes at least one amino acid mutation selected from L33F, L33P, L33Y, R135G, R135K, S159F, and S159Y. In some embodiments, an MGMT polypeptide that is more sensitive to inhibition by one or more MGMT inhibitors includes at least one amino acid mutation selected from L33F, R135G, R135K, and S159Y. In some embodiments, an MGMT polypeptide that is more sensitive to inhibition by one or more MGMT inhibitors includes at least one amino acid mutation selected from Y158F, G160A, and A170S. In some embodiments, an MGMT polypeptide that is more sensitive to inhibition by one or more MGMT inhibitors includes the amino acid mutation A170S.

In various embodiments, an inhibitor-resistant MGMT polypeptide of the present disclosure is characterized in that it can confer a proliferative advantage to HSCs in which it is expressed, following exposure to one or both of an alkylating agent and an MGMT inhibitor such as an O⁶-benzylguanine based inhibitor. In various embodiments, such proliferative advantage refers to comparative or competitive proliferation that is at least 10% (e.g., 20%, 30%, 40%, 50%, 100%, or more) greater than that of wild-type MGMT under same, comparable, and/or competitive conditions. In various embodiments, an inhibitor-resistant MGMT polypeptides of the present disclosure is characterized in that it does not confer a substantial proliferative disadvantage to HSCs in which it is expressed, e.g., in the absence of an MGMT inhibitor such as an O⁶-benzylguanine based inhibitor, and optionally in the presence of or absence of an alkylating agent. In various embodiments, such absence of a substantial proliferative disadvantage refers to comparative or competitive proliferation that is more than 5-fold (e.g., no more than 1.1, 1.2, 1.3, 1.4, 1.5, 1.6, 1.7, 1.8, 1.9, 2, 2.5, 3, 4 or 5-fold) less than that of wild-type MGMT under same, comparable, and/or competitive conditions

In various embodiments, an inhibitor-resistant MGMT is an MGMT polypeptide that includes at least one amino acid sequence difference as compared to a reference wild type sequence (i.e., includes at least one amino acid mutation) listed in Table 1 and/or Table 2 (e.g., 1, 2, 3, 4, 5, 6, 7, 8, 9, 10 or more amino acid mutations listed in Table 1 and/or Table 2). Amino acid mutations are presented in the format of (wild type amino acid(s)) (wild type amino acid position(s)) (amino acid(s) at corresponding position of mutant). For the avoidance of doubt, the term mutations is understood by those of skill in the art to include a difference as compared to a reference, and does not necessarily refer to, or imply, a change having occurred within any particular sequence of interest. Except as otherwise provided herein, reference to amino acid positions of an MGMT polypeptide refer to positions corresponding to the sequence of SEQ ID NO: 1.

In various embodiments, an inhibitor-resistant MGMT is an MGMT polypeptide that includes at least one amino acid mutation selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

In various embodiments, an inhibitor-resistant MGMT is an MGMT polypeptide that includes at least one amino acid mutation selected from L33F, L33K, L33P, L33R, L33W, L33Y, M134F, M134V, M134W, M134Y, R135G, R135K, R135L, R135T, N137D, N137F, N137P, P138K, G156I, G156P, G156V, Y158M, Y158W, S159F, S159I, S159L, S159P, S159T, S159W, S159Y, G160D, G160E, G160H, G160K, and G160P. In various embodiments, an inhibitor-resistant MGMT is an MGMT polypeptide that includes at least one amino acid mutation selected from L33F, L33K, L33P, L33R, L33W, L33Y, M134F, M134V, M134W, M134Y, R135G, R135K, R135L, R135T, N137D, N137F, N137P, P138K, G156I, G156P, G156V, Y158M, Y158W, G160D, G160E, G160H, G160K, and G160P. In various embodiments, an inhibitor-resistant MGMT is an MGMT polypeptide that includes at least one amino acid mutation selected from P140E, P140F, and P140H. In various embodiments, an inhibitor-resistant MGMT is an MGMT polypeptide that includes at least one amino acid mutation selected from S159F, S159I, S159L, S159P, S159T, S159W, and S159Y.

In various embodiments, an inhibitor-resistant MGMT is an MGMT polypeptide that includes at least one amino acid mutation selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140) PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK.

In various embodiments, an inhibitor-resistant MGMT is an MGMT polypeptide that includes the amino acid mutation P140K.

In various embodiments, an inhibitor-resistant MGMT does not include the amino acid mutation L33F, is not MGMT^{L33F}, and/or does not include the amino acid phenylalanine (F) at position 33. In various embodiments, an inhibitor-resistant MGMT does not include the amino acid mutation L33K, is not MGMT^{L33K}, and/or does not include the amino acid lysine (K) at position 33. In various embodiments, an inhibitor-resistant MGMT does not include the amino acid mutation L33P, is not MGMT^{L33P}, and/or does not include the amino acid proline (P) at position 33. In various embodiments, an inhibitor-resistant MGMT does not include the amino acid mutation L33R, is not MGMT^{L33R}, and/or does not include the amino acid arginine (R) at position 33. In various embodiments, an inhibitor-resistant MGMT does not include the amino acid mutation L33W, is not MGMT^{L33W}, and/or does not include the amino acid tryptophan (W) at position 33. In various embodiments, an inhibitor-resistant MGMT does not include the amino acid mutation L33Y, is not MGMT^{L33Y}, and/or does not include the amino acid tyrosine (Y) at position 33.

In various embodiments, an inhibitor-resistant MGMT does not include the amino acid mutation M134F, is not MGMT^{M134F}, and/or does not include the amino acid phenylalanine (F) at position 134. In various embodiments, an inhibitor-resistant MGMT does not include the amino acid mutation M134V, is not MGMT^{M134V}, and/or does not include the amino acid valine (V) at position 134. In various embodiments, an inhibitor-resistant MGMT does not include the amino acid mutation M134W, is not MGMT^{M134W}, and/or does not include the amino acid tryptophan (W) at position 134. In various embodiments, an inhibitor-resistant MGMT does not include the amino acid mutation M134Y, is not MGMT^{M134Y}, and/or does not include the amino acid tyrosine (Y) at position 134.

In various embodiments, an inhibitor-resistant MGMT does not include the amino acid mutation R135G, is not MGMT^{R135G}, and/or does not include the amino acid glycine (G) at position 135. In various embodiments, an inhibitor-resistant MGMT does not include the amino acid mutation R135K, is not MGMT^{R135K}, and/or does not include the amino acid lysine (K) at position 135. In various embodiments, an inhibitor-resistant MGMT does not include the amino acid mutation R135L, is not MGMT^{R135L}, and/or does not include the amino acid leucine (L) at position 135. In various embodiments, an inhibitor-resistant MGMT does not include the amino acid mutation R135T, is not MGMT^{R135T}, and/or does not include the amino acid threonine (T) at position 135.

In various embodiments, an inhibitor-resistant MGMT does not include the amino acid mutation N137D, is not MGMT^{N137D}, and/or does not include the amino acid aspartic acid (D) at position 137. In various embodiments, an inhibitor-resistant MGMT does not include the amino acid mutation N137F, is not MGMT^{N137F}, and/or does not include the amino acid phenylalanine (F) at position 137. In various embodiments, an inhibitor-resistant MGMT does not include the amino acid mutation N137P, is not MGMT^{N137P}, and/or does not include the amino acid proline (P) at position 137.

In various embodiments, an inhibitor-resistant MGMT does not include the amino acid mutation P138K, is not MGMT^{P138K}, and/or does not include the amino acid lysine (K) at position 138.

In various embodiments, an inhibitor-resistant MGMT does not include the amino acid mutation V139F, is not MGMT^{V139F}, and/or does not include the amino acid valine (V) at position 139.

In various embodiments, an inhibitor-resistant MGMT does not include the amino acid mutation P140E, is not MGMT^{P140E}, and/or does not include the amino acid glutamic acid (E) at position 140. In various embodiments, an inhibitor-resistant MGMT does not include the amino acid mutation P140F, is not MGMT^{P140F}, and/or does not include the amino acid phenylalanine (F) at position 140. In various embodiments, an inhibitor-resistant MGMT does not include the amino acid mutation P140H, is not MGMT^{P140H}, and/or does not include the amino acid histidine (H) at position 140.

In various embodiments, an inhibitor-resistant MGMT does not include the amino acid mutation P140A, is not MGMT^{P140A}, and/or does not include the amino acid alanine (A) at position 140. In various embodiments, an inhibitor-resistant MGMT does not include the amino acid mutation P140G, is not MGMT^{P140G}, and/or does not include the amino acid glycine (G) at position 140. In various embodiments, an inhibitor-resistant MGMT does not include the amino acid mutation P140I, is not MGMT^{P140I}, and/or does not include the amino acid isoleucine (I) at position 140. In various embodiments, an inhibitor-resistant MGMT does not include the amino acid mutation P140K, is not MGMT^{P140K}, and/or does not include the amino acid lysine (K) at position 140. In various embodiments, an inhibitor-resistant MGMT does not include the amino acid mutation P140L, is not MGMT^{P140L}, and/or does not include the amino acid leucine (L) at position 140. In various embodiments, an inhibitor-resistant MGMT does not include the amino acid mutation P140M, is not MGMT^{P140M}, and/or does not include the amino acid methionine (M) at position 140. In various embodiments, an inhibitor-resistant MGMT does not include the amino acid mutation P140N, is not MGMT^{P140N}, and/or does not include the amino acid asparagine (N) at position 140. In various embodiments, an inhibitor-resistant MGMT does not include the amino acid mutation P140Q, is not MGMT^{P140Q}, and/or does not include the amino acid glutamine (Q) at position 140. In various embodiments, an inhibitor-resistant MGMT does not include the amino acid mutation P140R, is not MGMT^{P140R}, and/or does not include the amino acid arginine (R) at position 140. In various embodiments, an inhibitor-resistant MGMT does not include the amino acid mutation P140S, is not MGMT^{P140S}, and/or does not include the amino acid serine(S) at position 140. In various embodiments, an inhibitor-resistant MGMT does not include the amino acid mutation P140T, is not MGMT^{P140T}, and/or does not include the amino acid threonine (T) at position 140.

In various embodiments, an inhibitor-resistant MGMT does not include the amino acid mutation L142M, is not MGMT^{L142M}, and/or does not include the amino acid methionine (M) at position 142.

In various embodiments, an inhibitor-resistant MGMT does not include the amino acid mutation C150Y, is not MGMT^{C150Y}, and/or does not include the amino acid tyrosine (Y) at position 150.

In various embodiments, an inhibitor-resistant MGMT does not include the amino acid mutation S152H, is not MGMT^{S152H}, and/or does not include the amino acid histidine (H) at position 152.

In various embodiments, an inhibitor-resistant MGMT does not include the amino acid mutation A154G, is not MGMT^{A154G}, and/or does not include the amino acid glycine (G) at position 154.

In various embodiments, an inhibitor-resistant MGMT does not include the amino acid mutation G156I, is not MGMT^{G156I}, and/or does not include the amino acid isoleucine (I) at position 156. In various embodiments, an inhibitor-resistant MGMT does not include the amino acid mutation G156P, is not MGMT^{G156P}, and/or does not include the amino acid proline (P) at position 156. In various embodiments, an inhibitor-resistant MGMT does not include the amino acid mutation G156V, is not MGMT^{G156V}, and/or does not include the amino acid valine (V) at position 156.

In various embodiments, an inhibitor-resistant MGMT does not include the amino acid mutation G156A, is not MGMT^{G156A}, and/or does not include the amino acid alanine (A) at position 156.

In various embodiments, an inhibitor-resistant MGMT does not include the amino acid mutation N157T, is not MGMT^{N157T}, and/or does not include the amino acid threonine (T) at position 157.

In various embodiments, an inhibitor-resistant MGMT does not include the amino acid mutation Y158M, is not MGMT^{Y158M}, and/or does not include the amino acid methionine (M) at position 158. In various embodiments, an inhibitor-resistant MGMT does not include the amino acid mutation Y158W, is not MGMT^{Y158W}, and/or does not include the amino acid tryptophan (W) at position 158.

In various embodiments, an inhibitor-resistant MGMT does not include the amino acid mutation Y158F, is not MGMT^{Y158F}, and/or does not include the amino acid phenylalanine (F) at position 158. In various embodiments, an inhibitor-resistant MGMT does not include the amino acid mutation Y158H, is not MGMT^{Y158H}, and/or does not include the amino acid histidine (H) at position 158.

In various embodiments, an inhibitor-resistant MGMT does not include the amino acid mutation S159F, is not MGMT^{S159F}, and/or does not include the amino acid phenylalanine (F) at position 159. In various embodiments, an inhibitor-resistant MGMT does not include the amino acid mutation S159I, is not MGMT^{S159I}, and/or does not include the amino acid isoleucine (I) at position 159. In various embodiments, an inhibitor-resistant MGMT does not include the amino acid mutation S159L, is not MGMT^{S159L}, and/or does not include the amino acid leucine (L) at position 159. In various embodiments, an inhibitor-resistant MGMT does not include the amino acid mutation S159P, is not MGMT^{S159P}, and/or does not include the amino acid proline (P) at position 159. In various embodiments, an inhibitor-resistant MGMT does not include the amino acid mutation S159T, is not MGMT^{S159T}, and/or does not include the amino acid threonine (T) at position 159. In various embodiments, an inhibitor-resistant MGMT does not include the amino acid mutation S159W, is not MGMT^{S159W}, and/or does not include the amino acid tryptophan (W) at position 159. In various embodiments, an inhibitor-resistant MGMT does not include the amino acid mutation S159Y, is not MGMT^{S159Y}, and/or does not include the amino acid tyrosine (Y) at position 159.

In various embodiments, an inhibitor-resistant MGMT does not include the amino acid mutation G160D, is not MGMT^{G160D}, and/or does not include the amino acid aspartic acid (D) at position 160. In various embodiments, an inhibitor-resistant MGMT does not include the amino acid mutation G160E, is not MGMT^{G160E}, and/or does not include the amino acid glutamic acid (E) at position 160. In various embodiments, an inhibitor-resistant MGMT does not include the amino acid mutation G160H, is not MGMT^{G160H}, and/or does not include the amino acid histidine (H) at position 160. In various embodiments, an inhibitor-resistant MGMT does not include the amino acid mutation G160K, is not MGMT^{G160K}, and/or does not include the amino acid lysine (K) at position 160. In various embodiments, an inhibitor-resistant MGMT does not include the amino acid mutation G160P, is not MGMT^{G160P}, and/or does not include the amino acid proline (P) at position 160.

In various embodiments, an inhibitor-resistant MGMT does not include the amino acid mutation G160A, is not MGMT^{G160A}, and/or does not include the amino acid alanine (A) at position 160. In various embodiments, an inhibitor-resistant MGMT does not include the amino acid mutation G160R, is not MGMT^{G160R}, and/or does not include the amino acid arginine (R) at position 160. In various embodiments, an inhibitor-resistant MGMT does not include the amino acid mutation G160S, is not MGMT^{G160S}, and/or does not include the amino acid serine(S) at position 160.

In various embodiments, an inhibitor-resistant MGMT does not include the amino acid mutation L162P, is not MGMT^{L162P}, and/or does not include the amino acid leucine (L) at position 162. In various embodiments, an inhibitor-resistant MGMT does not include the amino acid mutation L162V, is not MGMT^{L162V}, and/or does not include the amino acid valine (V) at position 162.

In various embodiments, an inhibitor-resistant MGMT does not include the amino acid mutation K165E, is not MGMT^{K165E}, and/or does not include the amino acid glutamic acid (E) at position 165. In various embodiments, an inhibitor-resistant MGMT does not include the amino acid mutation K165N, is not MGMT^{K165N}, and/or does not include the amino acid asparagine (N) at position 165. In various embodiments, an inhibitor-resistant MGMT does not include the amino acid mutation K165R, is not MGMT^{K165R}, and/or does not include the amino acid arginine (R) at position 165.

In various embodiments, an inhibitor-resistant MGMT does not include the amino acid mutation A170S, is not MGMT^{A170S}, and/or does not include the amino acid serine(S) at position 170.

In various embodiments, an inhibitor-resistant MGMT does not include the amino acid mutations PVP(138-140)CMK, in not MGMT^{PVP(138-140)CMK}, and/or does not include the amino acids cystine (C) at position 138, methionine (M) at position 139, and lysine (K) at position 140. In various embodiments, an inhibitor-resistant MGMT does not include the amino acid mutations PVP(138-140)CIK, in not MGMT^{PVP(138-140)CIK}, and/or does not include the amino acids cystine (C) at position 138, isoleucine (I) at position 139, and lysine (K) at position 140. In various embodiments, an inhibitor-resistant MGMT does not include the amino acid mutations PVP(138-140)HLK, in not MGMT^{PVP(138-140)HLK}, and/or does not include the amino acids histidine (H) at position 138, leucine (L) at position 139, and lysine (K) at position 140. In various embodiments, an inhibitor-resistant MGMT does not include the amino acid mutations PVP(138-140)KIK, in not MGMT^{PVP(138-140)KIK}, and/or does not include the amino acids lysine (K) at position 138, isoleucine (I) at position 139, and lysine (K) at position 140. In various embodiments, an inhibitor-resistant MGMT does not include the amino acid mutations PVP(138-140)KIR, in not MGMT^{PVP(138-140)KIR}, and/or does not include the amino acids lysine (K) at position 138, isoleucine (I) at position 139, and arginine (R) at position 140. In various embodiments, an inhibitor-resistant MGMT does not include the amino acid mutations PVP(138-140)KLK, in not MGMT^{PVP(138-140)KLK}, and/or does not include the amino acids lysine (K) at position 138, leucine (L) at position 139, and lysine (K) at position 140. In various embodiments, an inhibitor-resistant MGMT does not include the amino acid mutations PVP(138-140)KMK, in not MGMT^{PVP(138-140)KMK}, and/or does not include the amino acids lysine (K) at position 138, methionine (M) at position 139, and lysine (K) at position 140. In various embodiments, an inhibitor-resistant MGMT does not include the amino acid mutations PVP(138-140)KVK, in not MGMT^{PVP(138-140)KVK}, and/or does not include the amino acids lysine (K) at position 138, valine (V) at position 139, and lysine (K) at position 140. In various embodiments, an inhibitor-resistant MGMT does not include the amino acid mutations PVP(138-140)KWK, in not MGMT^{PVP(138-140)KWK}, and/or does not include the amino acids lysine (K) at position 138, tryptophan (W) at position 139, and lysine (K) at position 140. In various embodiments, an inhibitor-resistant MGMT does not include the amino acid mutations PVP(138-140)KYK, in not MGMT^{PVP(138-140)KYK}, and/or does not include the amino acids lysine (K) at position 138, tyrosine (Y) at position 139, and lysine (K) at position 140. In various embodiments, an inhibitor-resistant MGMT does not include the amino acid mutations PVP(138-140)KYN, in not MGMT^{PVP(138-140)KYN}, and/or does not include the amino acids lysine (K) at position 138, tyrosine (Y) at position 139, and asparagine (N) at position 140. In various embodiments, an inhibitor-resistant MGMT does not include the amino acid mutations PVP(138-140)KYR, in not MGMT^{PVP(138-140)KYR}, and/or does not include the amino acids lysine (K) at position 138, tyrosine (Y) at position 139, and arginine (R) at position 140. In various embodiments, an inhibitor-resistant MGMT does not include the amino acid mutations PVP(138-140)MIK, in not MGMT^{PVP(138-140)MIK}, and/or does not include the amino acids methionine (M) at position 138, isoleucine (I) at position 139, and lysine (K) at position 140. In various embodiments, an inhibitor-resistant MGMT does not include the amino acid mutations PVP(138-140)MLK, in not MGMT^{PVP(138-140)MLK}, and/or does not include the amino acids methionine (M) at position 138, leucine (L) at position 139, and lysine (K) at position 140. In various embodiments, an inhibitor-resistant MGMT does not include the amino acid mutations PVP(138-140)MMK, in not MGMT^{PVP(138-140)MMK}, and/or does not include the amino acids methionine (M) at position 138, methionine (M) at position 139, and lysine (K) at position 140. In various embodiments, an inhibitor-resistant MGMT does not include the amino acid mutations PVP(138-140)MVK, in not MGMT^{PVP(138-140)MVK}, and/or does not include the amino acids methionine (M) at position 138, valine (V) at position 139, and lysine (K) at position 140. In various embodiments, an inhibitor-resistant MGMT does not include the amino acid mutations PVP(138-140)MWK, in not MGMT^{PVP(138-140)MWK}, and/or does not include the amino acids methionine (M) at position 138, tryptophan (W) at position 139, and lysine (K) at position 140. In various embodiments, an inhibitor-resistant MGMT does not include the amino acid mutations PVP(138-140)MYR, in not MGMT^{PVP(138-140)MYR}, and/or does not include the amino acids methionine (M) at position 138, tyrosine (Y) at position 139, and arginine (R) at position 140. In various embodiments, an inhibitor-resistant MGMT does not include the amino acid mutations PVP(138-140)NIK, in not MGMT^{PVP(138-140)NIK}, and/or does not include the amino acids asparagine (N) at position 138, isoleucine (I) at position 139, and lysine (K) at position 140. In various embodiments, an inhibitor-resistant MGMT does not include the amino acid mutations PVP(138-140)NLK, in not MGMT^{PVP(138-140)NLK}, and/or does not include the amino acids asparagine (N) at position 138, leucine (L) at position 139, and lysine (K) at position 140. In various embodiments, an inhibitor-resistant MGMT does not include the amino acid mutations PVP(138-140)NLL, in not MGMT^{PVP(138-140)NLL}, and/or does not include the amino acids asparagine (N) at position 138, leucine (L) at position 139, and leucine (L) at position 140. In various embodiments, an inhibitor-resistant MGMT does not include the amino acid mutations PVP(138-140)PLK, in not MGMT^{PVP(138-140)PLK}, and/or does not include the amino acids proline (P) at position 138, leucine (L) at position 139, and lysine (K) at position 140. In various embodiments, an inhibitor-resistant MGMT does not include the amino acid mutations PVP(138-140)PYR, in not MGMT^{PVP(138-140)PYR}, and/or does not include the amino acids proline (P) at position 138, tyrosine (Y) at position 139, and arginine (R) at position 140. In various embodiments, an inhibitor-resistant MGMT does not include the amino acid mutations PVP(138-140)QLN, in not MGMT^{PVP(138-140)QLN}, and/or does not include the amino acids glutamine (Q) at position 138, leucine (L) at position 139, and asparagine (N) at position 140. In various embodiments, an inhibitor-resistant MGMT does not include the amino acid mutations PVP(138-140)RFK, in not MGMT^{PVP(138-140)RFK}, and/or does not include the amino acids arginine (R) at position 138, phenylalanine (F) at position 139, and lysine (K) at position 140. In various embodiments, an inhibitor-resistant MGMT does not include the amino acid mutations PVP(138-140)RTK, in not MGMT^{PVP(138-140)RTK}, and/or does not include the amino acids arginine (R) at position 138, threonine (T) at position 139, and lysine (K) at position 140. In various embodiments, an inhibitor-resistant MGMT does not include the amino acid mutations PVP(138-140)RYK, in not MGMT^{PVP(138-140)RYK}, and/or does not include the amino acids arginine (R) at position 138, tyrosine (Y) at position 139, and lysine (K) at position 140. In various embodiments, an inhibitor-resistant MGMT does not include the amino acid mutations PVP(138-140)SFK, in not MGMT^{PVP(138-140)SFK}, and/or does not include the amino acids serine(S) at position 138, phenylalanine (F) at position 139, and lysine (K) at position 140. In various embodiments, an inhibitor-resistant MGMT does not include the amino acid mutations PVP(138-140)SMK, in not MGMT^{PVP(138-140)SMK}, and/or does not include the amino acids serine(S) at position 138, methionine (M) at position 139, and lysine (K) at position 140. In various embodiments, an inhibitor-resistant MGMT does not include the amino acid mutations PVP(138-140)TIK, in not MGMT^{PVP(138-140)TIK}, and/or does not include the amino acids threonine (T) at position 138, isoleucine (I) at position 139, and lysine (K) at position 140. In various embodiments, an inhibitor-resistant MGMT does not include the amino acid mutations PVP(138-140)TLK, in not MGMT^{PVP(138-140)TLK}, and/or does not include the amino acids threonine (T) at position 138, leucine (L) at position 139, and lysine (K) at position 140. In various embodiments, an inhibitor-resistant MGMT does not include the amino acid mutations PVP(138-140)TLN, in not MGMT^{PVP(138-140)TLN}, and/or does not include the amino acids threonine (T) at position 138, leucine (L) at position 139, and asparagine (N) at position 140. In various embodiments, an inhibitor-resistant MGMT does not include the amino acid mutations PVP(138-140)TNK, in not MGMT^{PVP(138-140)TNK}, and/or does not include the amino acids threonine (T) at position 138, asparagine (N) at position 139, and lysine (K) at position 140. In various embodiments, an inhibitor-resistant MGMT does not include the amino acid mutations PVP(138-140)RCK, in not MGMT^{PVP(138-140)RCK}, and/or does not include the amino acids arginine (R) at position 138, cystine (C) at position 139, and lysine (K) at position 140. In various embodiments, an inhibitor-resistant MGMT does not include the amino acid mutations PVP(138-140)SYK, in not MGMT^{PVP(138-140)SYK}, and/or does not include the amino acids serine(S) at position 138, tyrosine (Y) at position 139, and lysine (K) at position 140. In various embodiments, an inhibitor-resistant MGMT does not include the amino acid mutations PVP(138-140)VMK, in not MGMT^{PVP(138-140)VMK}, and/or does not include the amino acids valine (V) at position 138, methionine (M) at position 139, and lysine (K) at position 140. In various embodiments, an inhibitor-resistant MGMT does not include the amino acid mutations PVP(138-140)YAK, in not MGMT^{PVP(138-140)YAK}, and/or does not include the amino acids tyrosine (Y) at position 138, alanine (A) at position 139, and lysine (K) at position 140.

The present disclosure includes DNA and mRNA coding sequences that encode an inhibitor-resistant MGMT (e.g., MGMT including at least one amino acid mutation set forth in Table 1 and/or Table 2). DNA encoding each amino acid mutation of an inhibitor-resistant MGMT polypeptide (and mRNA expressed therefrom), or mRNA encoding each amino acid mutation of an inhibitor-resistant MGMT polypeptide, can include a mutant DNA or mRNA codon that encodes the amino acid mutation. The present disclosure includes DNA and mRNA coding sequences that include at least one nucleic acid sequence difference as compared to a reference or wild type sequence (i.e., includes at least one nucleic acid sequence mutation) listed in Table 1 and/or Table 2 (e.g., 1, 2, 3, 4, 5, 6, 7, 8, 9, 10 or more nucleic acid sequence mutations listed in Table 1 and/or Table 2).

In various embodiments, a nucleic acid encoding an inhibitor-resistant MGMT does not encode a polypeptide that includes the amino acid mutation P140K, does not encode MGMT^{P140K}, and/or does not encode a polypeptide that includes the amino acid lysine (K) at position 140. In various embodiments, a nucleic acid encoding an inhibitor-resistant MGMT does not include a codon that corresponds to the amino acid K at position 140.

In various embodiments, a nucleic acid encoding an inhibitor-resistant MGMT encodes a polypeptide that includes at least one amino acid mutation selected from L33F, L33K, L33P, L33R, L33W, L33Y, M134F, M134V, M134W, M134Y, R135G, R135K, R135L, R135T, N137D, N137F. N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

In various embodiments, a nucleic acid encoding an inhibitor-resistant MGMT encodes a polypeptide that includes at least one amino acid mutation selected from L33F, L33K, L33P, L33R, L33W, L33Y, M134F, M134V, M134W, M134Y, R135G, R135K, R135L, R135T, N137D, N137F, N137P, P138K, G156I, G156P, G156V, Y158M, Y158W, S159F, S159I, S159L, S159P, S159T, S159W, S159Y, G160D, G160E, G160H, G160K, and G160P. In various embodiments, a nucleic acid encoding an inhibitor-resistant MGMT encodes a polypeptide that includes at least one amino acid mutation selected from P140E, P140F, and P140H.

In various embodiments, a nucleic acid encoding an inhibitor-resistant MGMT encodes a polypeptide that includes at least one amino acid mutation selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK.

In various embodiments, a nucleic acid encoding an inhibitor-resistant MGMT encodes a polypeptide that includes the amino acid mutation P140K.

In various embodiments, a nucleic acid encoding an inhibitor-resistant MGMT does not encode a polypeptide that includes the amino acid mutation L33F, does not encode MGMT^{L33F}, and/or does not encode a polypeptide that includes the amino acid phenylalanine (F) at position 33. In various embodiments, a nucleic acid encoding an inhibitor-resistant MGMT does not encode a polypeptide that includes the amino acid mutation L33K, does not encode MGMT^{L33K}, and/or does not encode a polypeptide that includes the amino acid lysine (K) at position 33. In various embodiments, a nucleic acid encoding an inhibitor-resistant MGMT does not encode a polypeptide that includes the amino acid mutation L33P, does not encode MGMT^{L33P}, and/or does not encode a polypeptide that includes the amino acid proline (P) at position 33. In various embodiments, a nucleic acid encoding an inhibitor-resistant MGMT does not encode a polypeptide that includes the amino acid mutation L33R, does not encode MGMT^{L33R}, and/or does not encode a polypeptide that includes the amino acid arginine (R) at position 33. In various embodiments, a nucleic acid encoding an inhibitor-resistant MGMT does not encode a polypeptide that includes the amino acid mutation L33W, does not encode MGMT^{L33W} 3W, and/or does not encode a polypeptide that includes the amino acid tryptophan (W) at position 33. In various embodiments, a nucleic acid encoding an inhibitor-resistant MGMT does not encode a polypeptide that includes the amino acid mutation L33Y, does not encode MGMT^{L33Y}, and/or does not encode a polypeptide that includes the amino acid tyrosine (Y) at position 33.

In various embodiments, an inhibitor-resistant MGMT does not encode a polypeptide that includes the amino acid mutation M134F, does not encode MGMT^{M134F}, and/or does not encode a polypeptide that includes the amino acid phenylalanine (F) at position 134. In various embodiments, a nucleic acid encoding an inhibitor-resistant MGMT does not encode a polypeptide that includes the amino acid mutation M134V, does not encode MGMT^{M134V}, and/or does not encode a polypeptide that includes the amino acid valine (V) at position 134. In various embodiments, a nucleic acid encoding an inhibitor-resistant MGMT does not encode a polypeptide that includes the amino acid mutation M134W, does not encode MGMT^{M134W}, and/or does not encode a polypeptide that includes the amino acid tryptophan (W) at position 134. In various embodiments, a nucleic acid encoding an inhibitor-resistant MGMT does not encode a polypeptide that includes the amino acid mutation M134Y, does not encode MGMT^{M134Y}, and/or does not encode a polypeptide that includes the amino acid tyrosine (Y) at position 134.

In various embodiments, an inhibitor-resistant MGMT does not encode a polypeptide that includes the amino acid mutation R135G, does not encode MGMT^{R135G}, and/or does not encode a polypeptide that includes the amino acid glycine (G) at position 135. In various embodiments, a nucleic acid encoding an inhibitor-resistant MGMT does not encode a polypeptide that includes the amino acid mutation R135K, does not encode MGMT^{R135K}, and/or does not encode a polypeptide that includes the amino acid lysine (K) at position 135. In various embodiments, a nucleic acid encoding an inhibitor-resistant MGMT does not encode a polypeptide that includes the amino acid mutation R135L, does not encode MGMT^{R135L}, and/or does not encode a polypeptide that includes the amino acid leucine (L) at position 135. In various embodiments, a nucleic acid encoding an inhibitor-resistant MGMT does not encode a polypeptide that includes the amino acid mutation R135T, does not encode MGMT^{R135T}, and/or does not encode a polypeptide that includes the amino acid threonine (T) at position 135.

In various embodiments, an inhibitor-resistant MGMT does not encode a polypeptide that includes the amino acid mutation N137D, does not encode MGMT^{N137D}, and/or does not encode a polypeptide that includes the amino acid aspartic acid (D) at position 137. In various embodiments, a nucleic acid encoding an inhibitor-resistant MGMT does not encode a polypeptide that includes the amino acid mutation N137F, does not encode MGMT^{N137F}, and/or does not encode a polypeptide that includes the amino acid phenylalanine (F) at position 137. In various embodiments, a nucleic acid encoding an inhibitor-resistant MGMT does not encode a polypeptide that includes the amino acid mutation N137P, does not encode MGMT^{N137P}, and/or does not encode a polypeptide that includes the amino acid proline (P) at position 137.

In various embodiments, an inhibitor-resistant MGMT does not encode a polypeptide that includes the amino acid mutation P138K, does not encode MGMT^{P138K}, and/or does not encode a polypeptide that includes the amino acid lysine (K) at position 138.

In various embodiments, an inhibitor-resistant MGMT does not encode a polypeptide that includes the amino acid mutation V139F, does not encode MGMT^{V139F}, and/or does not encode a polypeptide that includes the amino acid valine (V) at position 139.

In various embodiments, an inhibitor-resistant MGMT does not encode a polypeptide that includes the amino acid mutation P140E, does not encode MGMT^{P140E}, and/or does not encode a polypeptide that includes the amino acid glutamic acid (E) at position 140. In various embodiments, a nucleic acid encoding an inhibitor-resistant MGMT does not encode a polypeptide that includes the amino acid mutation P140F, does not encode MGMT^{P140F}, and/or does not encode a polypeptide that includes the amino acid phenylalanine (F) at position 140. In various embodiments, a nucleic acid encoding an inhibitor-resistant MGMT does not encode a polypeptide that includes the amino acid mutation P140H, does not encode MGMT^{P140H}, and/or does not encode a polypeptide that includes the amino acid histidine (H) at position 140.

In various embodiments, an inhibitor-resistant MGMT does not encode a polypeptide that includes the amino acid mutation P140A, does not encode MGMT^{P140A}, and/or does not encode a polypeptide that includes the amino acid alanine (A) at position 140. In various embodiments, a nucleic acid encoding an inhibitor-resistant MGMT does not encode a polypeptide that includes the amino acid mutation P140G, does not encode MGMT^{P140G}, and/or does not encode a polypeptide that includes the amino acid glycine (G) at position 140. In various embodiments, a nucleic acid encoding an inhibitor-resistant MGMT does not encode a polypeptide that includes the amino acid mutation P140I, does not encode MGMT^{P140I}, and/or does not encode a polypeptide that includes the amino acid isoleucine (I) at position 140. In various embodiments, a nucleic acid encoding an inhibitor-resistant MGMT does not encode a polypeptide that includes the amino acid mutation P140K, does not encode MGMT^{P140K}, and/or does not encode a polypeptide that includes the amino acid lysine (K) at position 140. In various embodiments, a nucleic acid encoding an inhibitor-resistant MGMT does not encode a polypeptide that includes the amino acid mutation P140L, does not encode MGMT^{P140L}, and/or does not encode a polypeptide that includes the amino acid leucine (L) at position 140. In various embodiments, a nucleic acid encoding an inhibitor-resistant MGMT does not encode a polypeptide that includes the amino acid mutation P140M, does not encode MGMT^{P140M}, and/or does not encode a polypeptide that includes the amino acid methionine (M) at position 140. In various embodiments, a nucleic acid encoding an inhibitor-resistant MGMT does not encode a polypeptide that includes the amino acid mutation P140N, does not encode MGMT^{P140N}, and/or does not encode a polypeptide that includes the amino acid asparagine (N) at position 140. In various embodiments, a nucleic acid encoding an inhibitor-resistant MGMT does not encode a polypeptide that includes the amino acid mutation P140Q, does not encode MGMT^{P140Q}, and/or does not encode a polypeptide that includes the amino acid glutamine (Q) at position 140. In various embodiments, a nucleic acid encoding an inhibitor-resistant MGMT does not encode a polypeptide that includes the amino acid mutation P140R, does not encode MGMT^{P140R}, and/or does not encode a polypeptide that includes the amino acid arginine (R) at position 140. In various embodiments, a nucleic acid encoding an inhibitor-resistant MGMT does not encode a polypeptide that includes the amino acid mutation P140S, does not encode MGMT^{P140S}, and/or does not encode a polypeptide that includes the amino acid serine(S) at position 140. In various embodiments, a nucleic acid encoding an inhibitor-resistant MGMT does not encode a polypeptide that includes the amino acid mutation P140T, does not encode MGMT^{P140T}, and/or does not encode a polypeptide that includes the amino acid threonine (T) at position 140.

In various embodiments, an inhibitor-resistant MGMT does not encode a polypeptide that includes the amino acid mutation L142M, does not encode MGMT^{L142M}, and/or does not encode a polypeptide that includes the amino acid methionine (M) at position 142.

In various embodiments, an inhibitor-resistant MGMT does not encode a polypeptide that includes the amino acid mutation C150Y, does not encode MGMT (150Y, and/or does not encode a polypeptide that includes the amino acid tyrosine (Y) at position 150.

In various embodiments, an inhibitor-resistant MGMT does not encode a polypeptide that includes the amino acid mutation S152H, does not encode MGMT^{S152H}, and/or does not encode a polypeptide that includes the amino acid histidine (H) at position 152.

In various embodiments, an inhibitor-resistant MGMT does not encode a polypeptide that includes the amino acid mutation A154G, does not encode MGMT^{A154G}, and/or does not encode a polypeptide that includes the amino acid glycine (G) at position 154.

In various embodiments, an inhibitor-resistant MGMT does not encode a polypeptide that includes the amino acid mutation G156I, does not encode MGMT^{G156I}, and/or does not encode a polypeptide that includes the amino acid isoleucine (I) at position 156. In various embodiments, a nucleic acid encoding an inhibitor-resistant MGMT does not encode a polypeptide that includes the amino acid mutation G156P, does not encode MGMT^{G156P}, and/or does not encode a polypeptide that includes the amino acid proline (P) at position 156. In various embodiments, a nucleic acid encoding an inhibitor-resistant MGMT does not encode a polypeptide that includes the amino acid mutation G156V, does not encode MGMT^{G156V}, and/or does not encode a polypeptide that includes the amino acid valine (V) at position 156.

In various embodiments, an inhibitor-resistant MGMT does not encode a polypeptide that includes the amino acid mutation G156A, does not encode MGMT^{G156A}, and/or does not encode a polypeptide that includes the amino acid alanine (A) at position 156.

In various embodiments, an inhibitor-resistant MGMT does not encode a polypeptide that includes the amino acid mutation N157T, does not encode MGMT^{N157T}, and/or does not encode a polypeptide that includes the amino acid threonine (T) at position 157.

In various embodiments, an inhibitor-resistant MGMT does not encode a polypeptide that includes the amino acid mutation Y158M, does not encode MGMT^{Y158M}, and/or does not encode a polypeptide that includes the amino acid methionine (M) at position 158. In various embodiments, a nucleic acid encoding an inhibitor-resistant MGMT does not encode a polypeptide that includes the amino acid mutation Y158W, does not encode MGMT^{Y158W}, and/or does not encode a polypeptide that includes the amino acid tryptophan (W) at position 158.

In various embodiments, an inhibitor-resistant MGMT does not encode a polypeptide that includes the amino acid mutation Y158F, does not encode MGMT^{Y158F}, and/or does not encode a polypeptide that includes the amino acid phenylalanine (F) at position 158. In various embodiments, a nucleic acid encoding an inhibitor-resistant MGMT does not encode a polypeptide that includes the amino acid mutation Y158H, does not encode MGMT^{Y158H}, and/or does not encode a polypeptide that includes the amino acid histidine (H) at position 158.

In various embodiments, an inhibitor-resistant MGMT does not encode a polypeptide that includes the amino acid mutation S159F, does not encode MGMT^{S159F}, and/or does not encode a polypeptide that includes the amino acid phenylalanine (F) at position 159. In various embodiments, a nucleic acid encoding an inhibitor-resistant MGMT does not encode a polypeptide that includes the amino acid mutation S159I, does not encode MGMT^{S159I}, and/or does not encode a polypeptide that includes the amino acid isoleucine (I) at position 159. In various embodiments, a nucleic acid encoding an inhibitor-resistant MGMT does not encode a polypeptide that includes the amino acid mutation S159L, does not encode MGMT^{S159L}, and/or does not encode a polypeptide that includes the amino acid leucine (L) at position 159. In various embodiments, a nucleic acid encoding an inhibitor-resistant MGMT does not encode a polypeptide that includes the amino acid mutation S159P, does not encode MGMT^{S159P}, and/or does not encode a polypeptide that includes the amino acid proline (P) at position 159. In various embodiments, a nucleic acid encoding an inhibitor-resistant MGMT does not encode a polypeptide that includes the amino acid mutation S159T, does not encode MGMT^{S159T}, and/or does not encode a polypeptide that includes the amino acid threonine (T) at position 159. In various embodiments, a nucleic acid encoding an inhibitor-resistant MGMT does not encode a polypeptide that includes the amino acid mutation S159W, does not encode MGMT^{S159W}, and/or does not encode a polypeptide that includes the amino acid tryptophan (W) at position 159. In various embodiments, a nucleic acid encoding an inhibitor-resistant MGMT does not encode a polypeptide that includes the amino acid mutation S159Y, does not encode MGMT^{S159Y}, and/or does not encode a polypeptide that includes the amino acid tyrosine (Y) at position 159.

In various embodiments, an inhibitor-resistant MGMT does not encode a polypeptide that includes the amino acid mutation G160D, does not encode MGMT^{G160D}, and/or does not encode a polypeptide that includes the amino acid aspartic acid (D) at position 160. In various embodiments, a nucleic acid encoding an inhibitor-resistant MGMT does not encode a polypeptide that includes the amino acid mutation G160E, does not encode MGMT^{G160E}, and/or does not encode a polypeptide that includes the amino acid glutamic acid (E) at position 160. In various embodiments, a nucleic acid encoding an inhibitor-resistant MGMT does not encode a polypeptide that includes the amino acid mutation G160H, does not encode MGMT^{G160H}, and/or does not encode a polypeptide that includes the amino acid histidine (H) at position 160. In various embodiments, a nucleic acid encoding an inhibitor-resistant MGMT does not encode a polypeptide that includes the amino acid mutation G160K, does not encode MGMT^{G160K}, and/or does not encode a polypeptide that includes the amino acid lysine (K) at position 160. In various embodiments, a nucleic acid encoding an inhibitor-resistant MGMT does not encode a polypeptide that includes the amino acid mutation G160P, does not encode MGMT^{G160P}, and/or does not encode a polypeptide that includes the amino acid proline (P) at position 160.

In various embodiments, an inhibitor-resistant MGMT does not encode a polypeptide that includes the amino acid mutation G160A, does not encode MGMT^{G160A}, and/or does not encode a polypeptide that includes the amino acid alanine (A) at position 160. In various embodiments, a nucleic acid encoding an inhibitor-resistant MGMT does not encode a polypeptide that includes the amino acid mutation G160R, does not encode MGMT^{G160R}, and/or does not encode a polypeptide that includes the amino acid arginine (R) at position 160. In various embodiments, a nucleic acid encoding an inhibitor-resistant MGMT does not encode a polypeptide that includes the amino acid mutation G160S, does not encode MGMT^{G160S}, and/or does not encode a polypeptide that includes the amino acid serine(S) at position 160.

In various embodiments, an inhibitor-resistant MGMT does not encode a polypeptide that includes the amino acid mutation L162P, does not encode MGMT^{L162P}, and/or does not encode a polypeptide that includes the amino acid leucine (L) at position 162. In various embodiments, a nucleic acid encoding an inhibitor-resistant MGMT does not encode a polypeptide that includes the amino acid mutation L162V, does not encode MGMT^{L162V}, and/or does not encode a polypeptide that includes the amino acid valine (V) at position 162.

In various embodiments, an inhibitor-resistant MGMT does not encode a polypeptide that includes the amino acid mutation K165E, does not encode MGMT^{K165E}, and/or does not encode a polypeptide that includes the amino acid glutamic acid (E) at position 165. In various embodiments, a nucleic acid encoding an inhibitor-resistant MGMT does not encode a polypeptide that includes the amino acid mutation K165N, does not encode MGMT^{K165N}, and/or does not encode a polypeptide that includes the amino acid asparagine (N) at position 165. In various embodiments, a nucleic acid encoding an inhibitor-resistant MGMT does not encode a polypeptide that includes the amino acid mutation K165R, does not encode MGMT^{K165R}, and/or does not encode a polypeptide that includes the amino acid arginine (R) at position 165.

In various embodiments, an inhibitor-resistant MGMT does not encode a polypeptide that includes the amino acid mutation A170S, does not encode MGMT^{A170S}, and/or does not encode a polypeptide that includes the amino acid serine(S) at position 170.

In various embodiments, an inhibitor-resistant MGMT does not encode a polypeptide that includes the amino acid mutations PVP(138-140)CMK, in not MGMT^{PVP(138-140)CMK}, and/or does not encode a polypeptide that includes the amino acids cystine (C) at position 138, methionine (M) at position 139, and lysine (K) at position 140. In various embodiments, a nucleic acid encoding an inhibitor-resistant MGMT does not encode a polypeptide that includes the amino acid mutations PVP(138-140)CIK, in not MGMT^{PVP(138-140)CIK}, and/or does not encode a polypeptide that includes the amino acids cystine (C) at position 138, isoleucine (I) at position 139, and lysine (K) at position 140. In various embodiments, a nucleic acid encoding an inhibitor-resistant MGMT does not encode a polypeptide that includes the amino acid mutations PVP(138-140)HLK, in not MGMT^{PVP(138-140)HLK}, and/or does not encode a polypeptide that includes the amino acids histidine (H) at position 138, leucine (L) at position 139, and lysine (K) at position 140. In various embodiments, a nucleic acid encoding an inhibitor-resistant MGMT does not encode a polypeptide that includes the amino acid mutations PVP(138-140)KIK, in not MGMT^{PVP(138-140)KIK}, and/or does not encode a polypeptide that includes the amino acids lysine (K) at position 138, isoleucine (I) at position 139, and lysine (K) at position 140. In various embodiments, a nucleic acid encoding an inhibitor-resistant MGMT does not encode a polypeptide that includes the amino acid mutations PVP(138-140)KIR, in not MGMT^{PVP(138-140)KIR}, and/or does not encode a polypeptide that includes the amino acids lysine (K) at position 138, isoleucine (I) at position 139, and arginine (R) at position 140. In various embodiments, a nucleic acid encoding an inhibitor-resistant MGMT does not encode a polypeptide that includes the amino acid mutations PVP(138-140)KLK, in not MGMT^{PVP(138-140)KLK}, and/or does not encode a polypeptide that includes the amino acids lysine (K) at position 138, leucine (L) at position 139, and lysine (K) at position 140. In various embodiments, a nucleic acid encoding an inhibitor-resistant MGMT does not encode a polypeptide that includes the amino acid mutations PVP(138-140)KMK, in not MGMT^{PVP(138-140)KMK}, and/or does not encode a polypeptide that includes the amino acids lysine (K) at position 138, methionine (M) at position 139, and lysine (K) at position 140. In various embodiments, a nucleic acid encoding an inhibitor-resistant MGMT does not encode a polypeptide that includes the amino acid mutations PVP(138-140)KVK, in not MGMT^{PVP(138-140)KVK}, and/or does not encode a polypeptide that includes the amino acids lysine (K) at position 138, valine (V) at position 139, and lysine (K) at position 140. In various embodiments, a nucleic acid encoding an inhibitor-resistant MGMT does not encode a polypeptide that includes the amino acid mutations PVP(138-140)KWK, in not MGMT^{PVP(138-140)KWK}, and/or does not encode a polypeptide that includes the amino acids lysine (K) at position 138, tryptophan (W) at position 139, and lysine (K) at position 140. In various embodiments, a nucleic acid encoding an inhibitor-resistant MGMT does not encode a polypeptide that includes the amino acid mutations PVP(138-140)KYK, in not MGMT^{PVP(138-140)KYK}, and/or does not encode a polypeptide that includes the amino acids lysine (K) at position 138, tyrosine (Y) at position 139, and lysine (K) at position 140. In various embodiments, a nucleic acid encoding an inhibitor-resistant MGMT does not encode a polypeptide that includes the amino acid mutations PVP(138-140)KYN, in not MGMT^{PVP(138-140)KYN}, and/or does not encode a polypeptide that includes the amino acids lysine (K) at position 138, tyrosine (Y) at position 139, and asparagine (N) at position 140. In various embodiments, a nucleic acid encoding an inhibitor-resistant MGMT does not encode a polypeptide that includes the amino acid mutations PVP(138-140)KYR, in not MGMT^{PVP(138-140)KYR}, and/or does not encode a polypeptide that includes the amino acids lysine (K) at position 138, tyrosine (Y) at position 139, and arginine (R) at position 140. In various embodiments, a nucleic acid encoding an inhibitor-resistant MGMT does not encode a polypeptide that includes the amino acid mutations PVP(138-140)MIK, in not MGMT^{PVP(138-140)MIK}, and/or does not encode a polypeptide that includes the amino acids methionine (M) at position 138, isoleucine (I) at position 139, and lysine (K) at position 140. In various embodiments, a nucleic acid encoding an inhibitor-resistant MGMT does not encode a polypeptide that includes the amino acid mutations PVP(138-140)MLK, in not MGMT^{PVP(138-140)MLK}, and/or does not encode a polypeptide that includes the amino acids methionine (M) at position 138, leucine (L) at position 139, and lysine (K) at position 140. In various embodiments, a nucleic acid encoding an inhibitor-resistant MGMT does not encode a polypeptide that includes the amino acid mutations PVP(138-140)MMK, in not MGMT^{PVP(138-140)MMK}, and/or does not encode a polypeptide that includes the amino acids methionine (M) at position 138, methionine (M) at position 139, and lysine (K) at position 140. In various embodiments, a nucleic acid encoding an inhibitor-resistant MGMT does not encode a polypeptide that includes the amino acid mutations PVP(138-140)MVK, in not MGMT^{PVP(138-140)MVK}, and/or does not encode a polypeptide that includes the amino acids methionine (M) at position 138, valine (V) at position 139, and lysine (K) at position 140. In various embodiments, a nucleic acid encoding an inhibitor-resistant MGMT does not encode a polypeptide that includes the amino acid mutations PVP(138-140)MWK, in not MGMT^{PVP(138-140)MWK}, and/or does not encode a polypeptide that includes the amino acids methionine (M) at position 138, tryptophan (W) at position 139, and lysine (K) at position 140. In various embodiments, a nucleic acid encoding an inhibitor-resistant MGMT does not encode a polypeptide that includes the amino acid mutations PVP(138-140)MYR, in not MGMT^{PVP(138-140)MYR}, and/or does not encode a polypeptide that includes the amino acids methionine (M) at position 138, tyrosine (Y) at position 139, and arginine (R) at position 140. In various embodiments, a nucleic acid encoding an inhibitor-resistant MGMT does not encode a polypeptide that includes the amino acid mutations PVP(138-140)NIK, in not MGMT^{PVP(138-140)NIK}, and/or does not encode a polypeptide that includes the amino acids asparagine (N) at position 138, isoleucine (I) at position 139, and lysine (K) at position 140. In various embodiments, a nucleic acid encoding an inhibitor-resistant MGMT does not encode a polypeptide that includes the amino acid mutations PVP(138-140)NLK, in not MGMT^{PVP(138-140)NLK}, and/or does not encode a polypeptide that includes the amino acids asparagine (N) at position 138, leucine (L) at position 139, and lysine (K) at position 140. In various embodiments, a nucleic acid encoding an inhibitor-resistant MGMT does not encode a polypeptide that includes the amino acid mutations PVP(138-140) NLL, in not MGMT^{PVP(138-140)NLL}, and/or does not encode a polypeptide that includes the amino acids asparagine (N) at position 138, leucine (L) at position 139, and leucine (L) at position 140. In various embodiments, a nucleic acid encoding an inhibitor-resistant MGMT does not encode a polypeptide that includes the amino acid mutations PVP(138-140)PLK, in not MGMT^{PVP(138-140)PLK}, and/or does not encode a polypeptide that includes the amino acids proline (P) at position 138, leucine (L) at position 139, and lysine (K) at position 140. In various embodiments, a nucleic acid encoding an inhibitor-resistant MGMT does not encode a polypeptide that includes the amino acid mutations PVP(138-140)PYR, in not MGMT^{PVP(138-140)PYR}, and/or does not encode a polypeptide that includes the amino acids proline (P) at position 138, tyrosine (Y) at position 139, and arginine (R) at position 140. In various embodiments, a nucleic acid encoding an inhibitor-resistant MGMT does not encode a polypeptide that includes the amino acid mutations PVP(138-140)QLN, in not MGMT^{PVP(138-140)QLN}, and/or does not encode a polypeptide that includes the amino acids glutamine (Q) at position 138, leucine (L) at position 139, and asparagine (N) at position 140. In various embodiments, a nucleic acid encoding an inhibitor-resistant MGMT does not encode a polypeptide that includes the amino acid mutations PVP(138-140)RFK, in not MGMT^{PVP(138-140)RFK}, and/or does not encode a polypeptide that includes the amino acids arginine (R) at position 138, phenylalanine (F) at position 139, and lysine (K) at position 140. In various embodiments, a nucleic acid encoding an inhibitor-resistant MGMT does not encode a polypeptide that includes the amino acid mutations PVP(138-140)RTK, in not MGMT^{PVP(138-140)RTK}, and/or does not encode a polypeptide that includes the amino acids arginine (R) at position 138, threonine (T) at position 139, and lysine (K) at position 140. In various embodiments, a nucleic acid encoding an inhibitor-resistant MGMT does not encode a polypeptide that includes the amino acid mutations PVP(138-140)RYK, in not MGMT^{PVP(138-140)RYK}, and/or does not encode a polypeptide that includes the amino acids arginine (R) at position 138, tyrosine (Y) at position 139, and lysine (K) at position 140. In various embodiments, a nucleic acid encoding an inhibitor-resistant MGMT does not encode a polypeptide that includes the amino acid mutations PVP(138-140)SFK, in not MGMT^{PVP(138-140)SFK}, and/or does not encode a polypeptide that includes the amino acids serine(S) at position 138, phenylalanine (F) at position 139, and lysine (K) at position 140. In various embodiments, a nucleic acid encoding an inhibitor-resistant MGMT does not encode a polypeptide that includes the amino acid mutations PVP(138-140)SMK, in not MGMT^{PVP(138-140)SMK}, and/or does not encode a polypeptide that includes the amino acids serine(S) at position 138, methionine (M) at position 139, and lysine (K) at position 140. In various embodiments, a nucleic acid encoding an inhibitor-resistant MGMT does not encode a polypeptide that includes the amino acid mutations PVP(138-140)TIK, in not MGMT^{PVP(138-140)TIK}, and/or does not encode a polypeptide that includes the amino acids threonine (T) at position 138, isoleucine (I) at position 139, and lysine (K) at position 140. In various embodiments, a nucleic acid encoding an inhibitor-resistant MGMT does not encode a polypeptide that includes the amino acid mutations PVP(138-140)TLK, in not MGMT^{PVP(138-140)TLK}, and/or does not encode a polypeptide that includes the amino acids threonine (T) at position 138, leucine (L) at position 139, and lysine (K) at position 140. In various embodiments, a nucleic acid encoding an inhibitor-resistant MGMT does not encode a polypeptide that includes the amino acid mutations PVP(138-140)TLN, in not MGMT^{PVP(138-140)TLN}, and/or does not encode a polypeptide that includes the amino acids threonine (T) at position 138, leucine (L) at position 139, and asparagine (N) at position 140. In various embodiments, a nucleic acid encoding an inhibitor-resistant MGMT does not encode a polypeptide that includes the amino acid mutations PVP(138-140)TNK, in not MGMT^{PVP(138-140)TNK}, and/or does not encode a polypeptide that includes the amino acids threonine (T) at position 138, asparagine (N) at position 139, and lysine (K) at position 140. In various embodiments, a nucleic acid encoding an inhibitor-resistant MGMT does not encode a polypeptide that includes the amino acid mutations PVP(138-140)RCK, in not MGMT^{PVP(138-140)RCK}, and/or does not encode a polypeptide that includes the amino acids arginine (R) at position 138, cysteine (C) at position 139, and lysine (K) at position 140. In various embodiments, a nucleic acid encoding an inhibitor-resistant MGMT does not encode a polypeptide that includes the amino acid mutations PVP(138-140)SYK, in not MGMT^{PVP(138-140)SYK}, and/or does not encode a polypeptide that includes the amino acids serine(S) at position 138, tyrosine (Y) at position 139, and lysine (K) at position 140. In various embodiments, a nucleic acid encoding an inhibitor-resistant MGMT does not encode a polypeptide that includes the amino acid mutations PVP(138-140)VMK, in not MGMT^{PVP(138-140)VMK}, and/or does not encode a polypeptide that includes the amino acids valine (V) at position 138, methionine (M) at position 139, and lysine (K) at position 140. In various embodiments, a nucleic acid encoding an inhibitor-resistant MGMT does not encode a polypeptide that includes the amino acid mutations PVP(138-140)YAK, in not MGMT^{PVP(138-140)YAK}, and/or does not encode a polypeptide that includes the amino acids tyrosine (Y) at position 138, alanine (A) at position 139, and lysine (K) at position 140.

Due to the redundancy of the genetic code, up to 6 codons can encode the same amino acid. Those of skill in the art will appreciate that any of the up to 6 codons that can encode a given mutant or non-mutant amino acid can be present at the corresponding codon position of a nucleic acid. Those of skill in the art will appreciate that amino acids are encoded by the codons of a coding sequence, such that a mutation of an MGMT coding sequence can include the presence of a codon that encodes an amino acid that differs from the amino acid encoded by a corresponding codon of a reference or wild type MGMT coding sequence.

TABLE 1
Inhibitor-Resistant MGMT Mutations

| Amino | WT | WT | | |
|---|---|---|---|---|
| Acid | Codon | Codon | Mutant Codon(s) | Mutant Codon(s) |
| Mutation | DNA | RNA | DNA | RNA |
| L33F | ctc | cuc | ttt, ttc (tty) | uuu, uuc (uuy) |
| L33K | ctc | cuc | aaa, aag (aar) | aaa, aag (aar) |
| L33P | ctc | cuc | cct, ccc, cca, ccg (ccn) | ccu, ccc, cca, ccg (ccn) |
| L33R | ctc | cuc | cgt, cgc, cga, cgg; aga, | cgu, cgc, cga, cgg; aga, |
| | | | agg (cgn, agr; or cgy, | agg (cgn, agr; or cgy, |
| | | | mgr) | mgr) |
| L33W | ctc | cuc | tgg | ugg |
| L33Y | ctc | cuc | tat, tac (tay) | uau, uac (uay) |
| M134F | atg | aug | ttt, ttc (tty) | uuu, uuc (uuy) |
| M134V | atg | aug | gtt, gtc, gta, gtg (gtn) | guu, guc, gua, gug (gun) |
| M134W | atg | aug | tgg | ugg |
| M134Y | atg | aug | tat, tac (tay) | uau, uac (uay) |
| R135G | aga | aga | ggt, ggc, gga, ggg (ggn) | ggu, ggc, gga, ggg (ggn) |
| R135K | aga | aga | aaa, aag (aar) | aaa, aag (aar) |
| R135L | aga | aga | ctt, ctc, cta, ctg; tta, ttg | cuu, cuc, cua, cug; uua, |
| | | | (ctn, ttr; or cty, ytr) | uug (cun, uur; or cuy, yur) |
| R135T | aga | aga | act, acc, aca, acg (acn) | acu, acc, aca, acg (acn) |
| N137D | aat | aau | gat, gac (gay) | gau, gac (gay) |
| N137F | aat | aau | ttt, ttc (tty) | uuu, uuc (uuy) |
| N137P | aat | aau | cct, ccc, cca, ccg (ccn) | ccu, ccc, cca, ccg (ccn) |
| P138K | cct | ccu | aaa, aag (aar) | aaa, aag (aar) |
| P140E | ccc | ccc | gaa, gag (gar) | gaa, gag (gar) |
| P140F | ccc | ccc | ttt, ttc (tty) | uuu, uuc (uuy) |
| P140H | ccc | ccc | cat, cac (cay) | cau, cac (cay) |
| G156I | ggc | ggc | att, atc, ata (ath) | auu, auc, aua (auh) |
| G156P | ggc | ggc | cct, ccc, cca, ccg (con) | ccu, ccc, cca, ccg (ccn) |
| G156V | ggc | ggc | gtt, gtc, gta, gtg (gtn) | guu, guc, gua, gug (gun) |
| Y158M | tac | uac | atg | aug |
| Y158W | tac | uac | tgg | ugg |
| S159F | tcc | ucc | ttt, ttc (tty) | uuu, uuc (uuy) |
| S159I | tcc | ucc | att, atc, ata (ath) | auu, auc, aua (auh) |
| S159L | tcc | ucc | ctt, ctc, cta, ctg; tta, ttg | cuu, cuc, cua, cug; uua, |
| | | | (ctn, ttr; or cty, ytr) | uug (cun, uur; or cuy, yur) |
| S159P | tcc | ucc | cct, ccc, cca, ccg (ccn) | ccu, ccc, cca, ccg (ccn) |
| S159T | tcc | ucc | act, acc, aca, acg (acn) | acu, acc, aca, acg (acn) |
| S159W | tcc | ucc | tgg | ugg |
| S159Y | tcc | ucc | tat, tac (tay) | uau, uac (uay) |
| G160D | gga | gga | gat, gac (gay) | gau, gac (gay) |
| G160E | gga | gga | gaa, gag (gar) | gaa, gag (gar) |
| G160H | gga | gga | cat, cac (cay) | cau, cac (cay) |
| G160K | gga | gga | aaa, aag (aar) | aaa, aag (aar) |
| G160P | gga | gga | cct, ccc, cca, ccg (ccn) | ccu, ccc, cca, ccg (ccn) |

TABLE 2
Additional Inhibitor-Resistant MGMT Mutations

| Amino | WT | WT | | |
|---|---|---|---|---|
| Acid | Codon | Codon | Mutant Codon(s) | Mutant Codon(s) |
| Mutation | DNA | RNA | DNA | RNA |
| V139F | gtc | guc | ttt, ttc (tty) | uuu, uuc (uuy) |
| P140A | ccc | ccc | gct, gcc, gca, gcg (gcn) | gcu, gcc, gca, gcg (gcn) |
| P140G | ccc | ccc | ggt, ggc, gga, ggg (ggn) | ggu, ggc, gga, ggg (ggn) |
| P140I | ccc | ccc | att, atc, ata (ath) | auu, auc, aua (auh) |
| P140K | ccc | ccc | aaa, aag (aar) | aaa, aag (aar) |
| P140L | ccc | ccc | ctt, ctc, cta, ctg; tta, ttg | cuu, cuc, cua, cug; uua, |
| | | | (ctn, ttr; or cty, ytr) | uug (cun, uur; or cuy, yur) |
| P140M | ccc | ccc | atg | aug |
| P140N | ccc | ccc | aat, aac (aay) | aau, aac (aay) |
| P140Q | ccc | ccc | caa, cag (car) | caa, cag (car) |
| P140R | ccc | ccc | cgt, cgc, cga, cgg; aga, | cgu, cgc, cga, cgg; aga, |
| | | | agg (cgn, agr; or cgy, | agg (cgn, agr; or cgy, |
| | | | mgr) | mgr) |
| P140S | ccc | ccc | tct, tcc, tca, tcg; agt, agc | ucu, ucc, uca, ucg; agu, |
| | | | (tcn, agy) | agc (ucn, agy) |
| P140T | ccc | ccc | act, acc, aca, acg (acn) | acu, acc, aca, acg (acn) |
| L142M | ctc | cuc | atg | aug |
| C150Y | tgc | ugc | tat, tac (tay) | uau, uac (uay) |
| S152H | agc | agc | cat, cac (cay) | cau, cac (cay) |
| A154G | gcc | gcc | ggt, ggc, gga, ggg (ggn) | ggu, ggc, gga, ggg (ggn) |
| G156A | ggc | ggc | gct, gcc, gca, gcg (gcn) | gcu, gcc, gca, gog (gcn) |
| N157T | aac | aac | act, acc, aca, acg (acn) | acu, acc, aca, acg (acn) |
| Y158F | tac | uac | ttt, ttc (tty) | uuu, uuc (uuy) |
| Y158H | tac | uac | cat, cac (cay) | cau, cac (cay) |
| G160A | gga | gga | gct, gcc, gca, gcg (gcn) | gcu, gcc, gca, gcg (gcn) |
| G160R | gga | gga | cgt, cgc, cga, cgg; aga, | cgu, cgc, cga, cgg; aga, |
| | | | agg (cgn, agr; or cgy, | agg (cgn, agr; or cgy, |
| | | | mgr) | mgr) |
| G160S | ga | gga | tct, tcc, tca, tcg; agt, agc | ucu, ucc, uca, ucg; agu, |
| | | | (tcn, agy) | agc (ucn, agy) |
| L162P | ctg | cug | cct, ccc, cca, ccg (ccn) | ccu, ccc, cca, ccg (ccn) |
| L162V | ctg | cug | gtt, gtc, gta, gtg (gtn) | guu, guc, gua, gug (gun) |
| K165E | aag | aag | gaa, gag (gar) | gaa, gag (gar) |
| K165N | aag | aag | aau, aac (aay) | aau, aac (aay) |
| K165R | aag | aag | cgt, cgc, cga, cgg; aga, | cgu, cgc, cga, cgg; aga, |
| | | | agg (cgn, agr; or cgy, | agg (cgn, agr; or cgy, |
| | | | mgr) | mgr) |
| A170S | gcc | gcc | tct, tcc, tca, tcg; agt, agc | ucu, ucc, uca, ucg; agu, |
| | | | (tcn, agy) | agc (ucn, agy) |
| PVP(138- | cct gtc | ccu guc | C: tgt, tgc (tgy) | C: ugu, ugc (ugy) |
| 140)CMK | ccc | ccc | M: atg | M: aug |
| | | | K: aaa, aag (aar) | K: aaa, aag (aar) |
| PVP(138- | cct gtc | ccu guc | C: tgt, tgc (tgy) | C: ugu, ugc (ugy) |
| 140)CIK | ccc | ccc | I: att, atc, ata (ath) | I: auu, auc, aua (auh) |
| | | | K: aaa, aag (aar) | K: aaa, aag (aar) |
| PVP(138- | cct gtc | ccu guc | H: cat, cac (cay) | H: cau, cac (cay) |
| 140)HLK | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | K: aaa, aag (aar) | K: aaa, aag (aar) |
| PVP(138- | cct gtc | ccu guc | K: aaa, aag (aar) | K: aaa, aag (aar) |
| 140)KIK | ccc | ccc | I: att, atc, ata (ath) | I: auu, auc, aua (auh) |
| | | | K: aaa, aag (aar) | K: aaa, aag (aar) |
| PVP(138- | cct gtc | ccu guc | K: aaa, aag (aar) | K: aaa, aag (aar) |
| 140)KIR | ccc | ccc | I: att, atc, ata (ath) | I: auu, auc, aua (auh) |
| | | | R: cgt, cgc, cga, cgg; aga, | R: cgu, cgc, cga, cgg; aga, |
| | | | agg (cgn, agr; or cgy, | agg (cgn, agr; or cgy, |
| | | | mgr) | mgr) |
| PVP(138- | cct gtc | ccu guc | K: aaa, aag (aar) | K: aaa, aag (aar) |
| 140)KLK | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | K: aaa, aag (aar) | K: aaa, aag (aar) |
| PVP(138- | cct gtc | ccu guc | K: aaa, aag (aar) | K: aaa, aag (aar) |
| 140)KMK | ccc | ccc | M: atg | M: aug |
| | | | K: aaa, aag (aar) | K: aaa, aag (aar) |
| PVP(138- | cct gtc | ccu guc | K: aaa, aag (aar) | K: aaa, aag (aar) |
| 140)KVK | ccc | ccc | V: gtt, gtc, gta, gtg (gtn) | V: guu, guc, gua, gug |
| | | | K: aaa, aag (aar) | (gun) |
| | | | | K: aaa, aag (aar) |
| PVP(138- | cct gtc | ccu guc | K: aaa, aag (aar) | K: aaa, aag (aar) |
| 140)KWK | ccc | ccc | W: tgg | W: ugg |
| | | | K: aaa, aag (aar) | K: aaa, aag (aar) |
| PVP(138- | cct gtc | ccu guc | K: aaa, aag (aar) | K: aaa, aag (aar) |
| 140)KYK | ccc | ccc | Y: tat, tac (tay) | Y: uau, uac (uay) |
| | | | K: aaa, aag (aar) | K: aaa, aag (aar) |
| PVP(138- | cct gtc | ccu guc | K: aaa, aag (aar) | K: aaa, aag (aar) |
| 140)KYN | ccc | ccc | Y: tat, tac (tay) | Y: uau, uac (uay) |
| | | | N: aat, aac (aay) | N: aau, aac (aay) |
| PVP(138- | cct gtc | ccu guc | K: aaa, aag (aar) | K: aaa, aag (aar) |
| 140)KYR | ccc | ccc | Y: tat, tac (tay) | Y: uau, uac (uay) |
| | | | R: cgt, cgc, cga, cgg; aga, | R: cgu, cgc, cga, cgg; aga, |
| | | | agg (cgn, agr; or cgy, | agg (cgn, agr; or cgy, |
| | | | mgr) | mgr) |
| PVP(138- | cct gtc | ccu guc | M: atg | M: aug |
| 140)MIK | ccc | ccc | I: att, atc, ata (ath) | I: auu, auc, aua (auh) |
| | | | K: aaa, aag (aar) | K: aaa, aag (aar) |
| PVP(138- | cct gtc | ccu guc | M: atg | M: aug |
| 140)MLK | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | K: aaa, aag (aar) | K: aaa, aag (aar) |
| PVP(138- | cct gtc | ccu guc | M: atg | M: aug |
| 140)MMK | ccc | ccc | M: atg | M: aug |
| | | | K: aaa, aag (aar) | K: aaa, aag (aar) |
| PVP(138- | cct gtc | ccu guc | M: atg | M: aug |
| 140)MVK | ccc | ccc | V: gtt, gtc, gta, gtg (gtn) | V: guu, guc, gua, gug |
| | | | K: aaa, aag (aar) | (gun) |
| | | | | K: aaa, aag (aar) |
| PVP(138- | cct gtc | ccu guc | M: atg | M: aug |
| 140)MWK | ccc | ccc | W: tgg | W: ugg |
| | | | K: aaa, aag (aar) | K: aaa, aag (aar) |
| PVP(138- | cct gtc | ccu guc | M: atg | M: aug |
| 140)MYR | ccc | ccc | Y: tat, tac (tay) | Y: uau, uac (uay) |
| | | | R: cgt, cgc, cga, cgg; aga, | R: cgu, cgc, cga, cgg; aga, |
| | | | agg (cgn, agr; or cgy, | agg (cgn, agr; or cgy, |
| | | | mgr) | mgr) |
| PVP(138- | cct gtc | ccu guc | N: aat, aac (aay) | N: aau, aac (aay) |
| 140)NIK | ccc | ccc | I: att, atc, ata (ath) | I: auu, auc, aua (auh) |
| | | | K: aaa, aag (aar) | K: aaa, aag (aar) |
| PVP(138- | cct gtc | ccu guc | N: aat, aac (aay) | N: aau, aac (aay) |
| 140)NLK | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | K: aaa, aag (aar) | K: aaa, aag (aar) |
| PVP(138- | cct gtc | ccu guc | N: aat, aac (aay) | N: aau, aac (aay) |
| 140)NLL | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | L: ctt, ctc, cta, ctg; tta, ttg | L: cuu, cuc, cua, cug; uua, |
| | | | (ctn, ttr; or cty, ytr) | uug (cun, uur; or cuy, yur) |
| PVP(138- | cct gtc | ccu guc | P: cct, ccc, cca, ccg (ccn) | P: ccu, ccc, cca, ccg (ccn) |
| 140)PLK | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | K: aaa, aag (aar) | K: aaa, aag (aar) |
| PVP(138- | cct gtc | ccu guc | P: cct, ccc, cca, ccg (ccn) | P: ccu, ccc, cca, ccg (ccn) |
| 140)PYR | ccc | ccc | Y: tat, tac (tay) | Y: uau, uac (uay) |
| | | | R: cgt, cgc, cga, cgg; aga, | R: cgu, cgc, cga, cgg; aga, |
| | | | agg (cgn, agr; or cgy, | agg (cgn, agr; or cgy, |
| | | | mgr) | mgr) |
| PVP(138- | cct gtc | ccu guc | Q: caa, cag (car) | Q: caa, cag (car) |
| 140)QLN | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | N: aat, aac (aay) | N: aau, aac (aay) |
| PVP(138- | cct gtc | ccu guc | R: cgt, cgc, cga, cgg; aga, | R: cgu, cgc, cga, cgg; aga, |
| 140)RFK | ccc | ccc | agg (cgn, agr; or cgy, | agg (cgn, agr; or cgy, |
| | | | mgr) | mgr) |
| | | | F: ttt, ttc (tty) | F: uuu, uuc (uuy) |
| | | | K: aaa, aag (aar) | K: aaa, aag (aar) |
| PVP(138- | cct gtc | ccu guc | R: cgt, cgc, cga, cgg; aga, | R: cgu, cgc, cga, cgg; aga, |
| 140)RTK | ccc | ccc | agg (cgn, agr; or cgy, | agg (cgn, agr; or cgy, |
| | | | mgr) | mgr) |
| | | | T: act, acc, aca, acg (acn) | T: acu, acc, aca, acg (acn) |
| | | | K: aaa, aag (aar) | K: aaa, aag (aar) |
| PVP(138- | cct gtc | ccu guc | R: cgt, cgc, cga, cgg; aga, | R: cgu, cgc, cga, cgg; aga, |
| 140)RYK | ccc | ccc | agg (cgn, agr; or cgy, | agg (cgn, agr; or cgy, |
| | | | mgr) | mgr) |
| | | | Y: tat, tac (tay) | Y: uau, uac (uay) |
| | | | K: aaa, aag (aar) | K: aaa, aag (aar) |
| PVP(138- | cct gtc | ccu guc | S: tct, tcc, tca, tcg; agt, | S: ucu, ucc, uca, ucg; agu, |
| 140)SFK | ccc | ccc | agc (tcn, agy) | agc (ucn, agy) |
| | | | F: ttt, ttc (tty) | F: uuu, uuc (uuy) |
| | | | K: aaa, aag (aar) | K: aaa, aag (aar) |
| PVP(138- | cct gtc | ccu guc | S: tct, tcc, tca, tcg; agt, | S: ucu, ucc, uca, ucg; agu, |
| 140)SMK | ccc | ccc | agc (tcn, agy) | agc (ucn, agy) |
| | | | M: atg | M: aug |
| | | | K: aaa, aag (aar) | K: aaa, aag (aar) |
| PVP(138- | cct gtc | ccu guc | T: act, acc, aca, acg (acn) | T: acu, acc, aca, acg (acn) |
| 140)TIK | ccc | ccc | I: att, atc, ata (ath) | I: auu, auc, aua (auh) |
| | | | K: aaa, aag (aar) | K: aaa, aag (aar) |
| PVP(138- | cct gtc | ccu guc | T: act, acc, aca, acg (acn) | T: acu, acc, aca, acg (acn) |
| 140)TLK | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | K: aaa, aag (aar) | K: aaa, aag (aar) |
| PVP(138- | cct gtc | ccu guc | T: act, acc, aca, acg (acn) | T: acu, acc, aca, acg (acn) |
| 140)TLN | ccc | ccc | L: ctt, ctc, cta, ctg; tta, ttg | L: cuu, cuc, cua, cug; uua, |
| | | | (ctn, ttr; or cty, ytr) | uug (cun, uur; or cuy, yur) |
| | | | N: aat, aac (aay) | N: aau, aac (aay) |
| PVP(138- | cct gtc | ccu guc | T: act, acc, aca, acg (acn) | T: acu, acc, aca, acg (acn) |
| 140)TNK | ccc | ccc | N: aat, aac (aay) | N: aau, aac (aay) |
| | | | K: aaa, aag (aar) | K: aaa, aag (aar) |
| PVP(138- | cct gtc | ccu guc | R: cgt, cgc, cga, cgg; aga, | R: cgu, cgc, cga, cgg; aga, |
| 140)RCK | ccc | ccc | agg (cgn, agr; or cgy, | agg (cgn, agr; or cgy, |
| | | | mgr) | mgr) |
| | | | C: tgt, tgc (tgy) | C: ugu, ugc (ugy) |
| | | | K: aaa, aag (aar) | K: aaa, aag (aar) |
| PVP(138- | cct gtc | ccu guc | S: tct, tcc, tca, tcg; agt, | S: ucu, ucc, uca, ucg; agu, |
| 140)SYK | ccc | ccc | agc (tcn, agy) | agc (ucn, agy) |
| | | | Y: tat, tac (tay) | Y: uau, uac (uay) |
| | | | K: aaa, aag (aar) | K: aaa, aag (aar) |
| PVP(138- | cct gtc | ccu guc | V: gtt, gtc, gta, gtg (gtn) | V: guu, guc, gua, gug |
| 140)VMK | ccc | ccc | M: atg | (gun) |
| | | | K: aaa, aag (aar) | M: aug |
| | | | | K: aaa, aag (aar) |
| PVP(138- | cct gtc | ccu guc | Y: tat, tac (tay) | Y: uau, uac (uay) |
| 140)YAK | ccc | ccc | A: gct, gcc, gca, gcg | A: gcu, gcc, gca, gcg |
| | | | (gcn) | (gcn) |
| | | | K: aaa, aag (aar) | K: aaa, aag (aar) |

In various embodiments, MGMT^{P140K} refers to an inhibitor-resistant MGMT polypeptide that differs from a reference MGMT polypeptide only in that it includes the amino acid K at position 140 corresponding to SEQ ID NO: 1. In particular embodiments, MGMT^{P140K} refers specifically to a polypeptide including and/or consisting essentially of and/or consisting of SEQ ID NO: 8.

SEQ ID NO: 8
MDKDCEMKRTTLDSPLGKLELSGCEQGLHEIKLLGKGTSAADAVE
VPAPAAVLGGPEPLMQCTAWLNAYFHQPEAIEEFPVPALHHPVFQ
QESFTRQVLWKLLKVVKFGEVISYQQLAALAGNPKAARAVGGAMR
GNPVKILIPCHRVVCSSGAVGNYSGGLAVKEWLLAHEGHRLGKPG
LGGSSGLAGAWLKGAGATSGSPPAGRN

The present disclosure includes inhibitor-resistant MGMT sequences other than inhibitor-resistant MGMT sequences that include the mutation P140K (e.g., MGMT^{P140K}) and nucleic acid sequences encoding the same. The present disclosure includes inhibitor-resistant MGMT sequences other than inhibitor-resistant MGMT sequences that include V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and/or PVP(138-140)YAK, and nucleic acid sequences encoding the same. The present disclosure includes inhibitor-resistant MGMT sequences other than inhibitor-resistant MGMT sequences that include L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P, and nucleic acid sequences encoding the same. The present disclosure includes inhibitor-resistant MGMT sequences other than inhibitor-resistant MGMT sequences that differ from a reference MGMT polypeptide sequence only by the mutation P140K, and nucleic acid sequences encoding the same. In various embodiments, an inhibitor-resistant MGMT does not include the amino acid mutation P140K.

### MGMT Inhibitors

The present disclosure includes inhibitor-resistant MGMT polypeptides. An inhibitor-resistant MGMT polypeptide can be resistant to any of one or more MGMT inhibitors.

In various embodiments, an MGMT inhibitor is or includes O⁶-meG or an analog or derivative thereof. In various embodiments, an MGMT inhibitor is or includes O⁶-benzylguanine (O⁶BG) or an analog or derivative thereof. O⁶BG donates an alkyl group to MGMT, inactivating it and initiating degradation. In various embodiments, an analog or derivative of O⁶-meG and/or O⁶BG can inhibit MGMT through alkyl group transfer. In various embodiments, an analog or derivative of O⁶-meG and/or O⁶BG can be or include O⁶-(3-bromobenzyl) guanine, O⁶-2-fluoropyridinylmethylguanine (O⁶FPG), O6-3-iodobenzylguanine (O⁶IBG), O⁶-(4-bromothenyl) guanine (O⁶BTG; PaTrin-2), O⁶-5-iodothenylguanine (O⁶ITG), 8-aza-O⁶-benzylguanine (8-aza-BG), O⁶-benzyl-8-bromoguanine (8-bromo-BG), 2-amino-4-benzyloxy-5-nitropyrimidine (4-desamino-5-nitro-BP), O⁶-[p-(hydroxymethyl)benzyl] guanine (HN-BG), O⁶-benzyl-8-methylguanine (8-methyl-BG), O⁶-benzyl-7, 8-dihydro-8-oxoguanine (8-oxo-BG), 2,4,5,-triamino-6-benzyloxyprimidine (5-amino-BP), O⁶-benzyl-9-[(3-oxo-5 α-androstan-17β-yloxycarbonyl)methyl] guanine (DHT-BG), O⁶-benzyl-9-(3-oxo-4-androsten-17β-yloxycarbonyl)methyl] guanine (AND-BG), and/or 8-amino-O⁶-benzylguanine (8-amino-BG).

In various embodiments, an MGMT inhibitor is or includes diethylamine NONOate, Lomeguatrib, 2,4-diamino-6-benzyloxy-5-nitrosopyrimidine (5-nitroso-BP), and/or 2,4-diamino-6-benzyloxy-5-nitropyrimidine (5-nitro-BP).

In various embodiments, an MGMT inhibitor is associated (e.g., conjugated) with a further agent, e.g., a targeting agent. In various embodiments, a targeting agent is a polypeptide such as an antibody or polypeptide ligand of a receptor. In various embodiments, a targeting agent is a small molecule such as a small molecule ligand of a receptor. Accordingly, reference to MGMT inhibitors provided herein includes without limitation agents that include an agent that inhibits MGMT (e.g., is independently capable of inhibitor MGMT) associated (e.g., conjugated) with an agent that does not inhibit MGMT (e.g., an agent that is not independently capable of inhibiting MGMT) and/or a targeting agent or otherwise functional agent.

### Alkylating Agents

Exposure to MGMT inhibitors can sensitize cells that express wild type MGMT to elimination by alkylating agents. Various alkylating agents are known in the art. Alkylating agents include, without limitation, a nitrosoureas (e.g., nimustine (ACNU), carmustine (bis-chloroethylnitrosourea (BCNU), lomustine (CCNU), streptozocin, or semustine (methyl CCNU)), a nitrogen mustard (e.g., mechlorethamine, cyclophosphamide, ifosfamide, melphalan, chlormethine, ramustine or uracil mustard, bendamustine, or chlorambucil), ethylenamine and methylenamine derivatives (e.g., altretamine, thiotepa), an aziridine or epoxide (e.g., thiotepa, mitomycin C, or diaziquone (AZQ)), and alkyl sulfonate (e.g., busulfan or hepsulfam), a triazine or hydrazine (e.g., mitozolomide, temozolomide, procarbazine, or dacarbazine), hexamethylmelamine, and chlorambucil.

As one example of an alkylating agent, BCNU promotes DNA alkylation at the 06 position of guanine, leading to DNA interstrand crosslinking and altered fidelity of DNA replication and transcription. This induced interstrand crosslinking involves formation of a chloroethyl adduct at the guanine residue that undergoes an intramolecular rearrangement to produce an unstable intermediate that reacts with the cross strand cytosine residue. The result is an N′-guanine, N3-cytosine-ethanol crosslink.

### Editing Enzymes and Systems for Modification of Nucleic Acids Encoding MGMT

The present disclosure includes editing of MGMT-encoding nucleic acids to produce a modified MGMT-encoding nucleic acid that encodes an inhibitor-resistant MGMT polypeptide. As provided herein, editing includes any modification of a nucleic acid that results in a difference in nucleic acid sequence. Editing agents refer to molecules that can be delivered to a cell or system to cause or contribute to editing. An editing system refers to two or more editing agents that are together sufficient to cause editing (e.g., a base editor and a guide RNA or a prime editor and a guide RNA) or to a single editing agent alone sufficient to cause editing. Editing systems of the present disclosure can include at least one editing agent that includes an editing enzyme. An editing agent can be a fusion polypeptide that includes an editing enzyme. The present disclosure includes a variety of editing agents and editing systems capable of editing MGMT-encoding nucleic acids. As those of skill in the art will appreciate, many or all editing agents described herein can be targeted to induce particular changes using approaches known to those of skill in the art.

### Base Editing Enzymes and Systems for Modification of Nucleic Acids Encoding MGMT

The present disclosure includes editing systems that utilize a deaminase (e.g., a base editing system) for editing of nucleic acid targets, including in various embodiments modification an MGMT-encoding nucleic acid to produce a nucleic acid encoding inhibitor-resistant MGMT. In various embodiments, an editing agent can include an editing enzyme that includes a deaminase. Deamination is the removal of an amine group from a molecule such as a nucleotide of a nucleic acid. Deamination of a nucleotide can cause changes in the sequence of a nucleic acid, and deaminases are useful in editing for at least that reason. Deamination of adenosine (A) yields inosine (I), which has the same base pairing preferences as a guanosine in DNA and is thus recognized by cell replication machinery as guanosine, resulting in an A-T to G-C transition. Deamination of cytosine (C) yields uridine (U), which is recognized by cell replication machinery as thymine, resulting in a C-G to T-A transition. Collectively, cytosine and adenosine deamination can be used to cause transitions from A to G, T to C, C to T, or G to A. Other deaminase activities are also known. For example, deamination of 5-methylcytosine yields thymine and deamination of guanosine yields xanthine, though xanthine, like guanosine, pairs with cytosine. Deaminases that deaminate cytosine can be referred to as cytosine deaminases. Deaminases that deaminate adenosine can be referred to as adenosine deaminases.

In particular embodiments, a base editing enzyme includes a cytidine deaminase domain or an adenine deaminase domain. Certain embodiments utilize a cytidine deaminase domain as the nucleobase deaminase enzyme. Particular embodiments utilize an adenine deaminase domain as the nucleobase deaminase enzyme.

Examples of cytosine deaminase enzymes (CBEs) include APOBEC1, APOBEC3A, APOBEC3G, evoAPOBEC, BE4-YE1, CDA1, and AID. APOBEC1 particularly accepts single-stranded (ss)DNA as a substrate but is incapable of acting on double-stranded (ds)DNA.

For adenosine base editors (ABEs), exemplary adenosine deaminases that can act on DNA for adenine base editing include a mutant TadA adenosine deaminases (TadA*) that accepts DNA as its substrate. E. coli TadA typically acts as a homodimer to deaminate adenosine in transfer RNA (tRNA). TadA* deaminase catalyzes the conversion of a target ‘A’ to ‘I’ (inosine), which is treated as ‘G’ by cellular polymerases. Subsequently, an original genomic A-T base pair can be converted to a G-C pair. As the cellular inosine excision repair is not as active as uracil excision, ABE does not require any additional inhibitor protein like UGI in CBE. In some embodiments, an ABE can include one or more, or all, of three components including a wild-type E. coli tRNA-specific adenosine deaminase (TadA) monomer, which can play a structural role during base editing, a TadA* mutant TadA monomer that catalyzes deoxyadenosine deamination, and/or a Cas nickase such as Cas9(D10A). In certain embodiments, there is a linker positioned between TadA and TadA*, and in certain embodiments there is a linker positioned between TadA* and the Cas nickase. In various embodiments, one or both linkers includes at least 6 amino acids, e.g., at least 5, 6, 7, 8, 9, 10, 15, 20, 25, 30, 35, 40, 45, or 50 amino acids (e.g., having a lower bound of 5, 6, 7, 8, 9, 10, or 15, amino acids and an upper bound of 20, 25, 30, 35, 40, 45, or 50 amino acids). In various embodiments, one or both linkers include 32 amino acids. In some embodiments, one or both linkers has a sequence according to (SGGS)₂-XTEN-(SGGS)₂ (i.e., SGGSSGGSSGSETPGTSESATPESSGGSSGGS) (SEQ ID NO: 9) or a sequence otherwise known to those of skill in the art.

In various embodiments, an editing system includes a deaminase associated with a DNA binding domain such as a catalytically impaired nuclease domain. In various embodiments, the DNA binding domain can localize the deaminase to a target nucleic acid in which one or more nucleotides are deaminated by the deaminase. Catalytically impaired nuclease domains are polypeptide domains that have amino acid sequences engineered from reference nuclease domain sequences but that have a reduced ability to cause double-strand breaks (DSBs) as compared to the reference (e.g., a wild type and/or fully functional nuclease) or have no ability to cause double-strand breaks. As referred to herein, a nickase refers to a catalytically impaired nuclease domain that, upon contact with a double-stranded nucleic acid substrate, cleaves one strand (e.g., a target strand) of the double-stranded nucleic acid but not both strands of the double-stranded nucleic acid. In various embodiments, a nickase, upon contact with a double-stranded nucleic acid substrate, cleaves one strand of the double-stranded nucleic acid but not both strands of the double-stranded nucleic acid in at least 70% of contacted double-stranded nucleic acid substrates (e.g., at least 70%, 75%, 80%, 85%, 90%, 91%, 92%, 93%, 94%, 95%, 96%, 97%, 98%, 99%, or 100% of double-stranded nucleic acid substrates).

Base editing systems are exemplary of editing systems that include deaminase enzymes. A base editing enzyme includes a deaminase enzyme fused to a DNA binding domain that is a catalytically impaired nuclease domain (e.g., a nickase, e.g., a nickase that nicks a single strand, e.g., a non-edited strand). DNA binding domains of base editing enzymes can be RNA guided DNA binding domains, in that an RNA guide can direct the DNA binding domain to a target nucleic acid sequence. Catalytically impaired nuclease domains of a base editing enzyme can bind nucleic acids and can localize the deaminase enzyme to a target nucleic acid.

Any nuclease of the CRISPR system can be engineered to produce a catalytically impaired nuclease domain (e.g., a nickase) and used within a base editing enzyme or system. Exemplary Cas nucleases include Cas1, Cas1B, Cas2, Cas3, Cas4, Cas5, Cas6, Cas7, Cas8, Cas9 (also known as Csn1 or Csx12), Cas10, CasX, CasY, C2c3, C2c2 and C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, and variants thereof. Numerous forms and variants of Cas nucleases are known in the art (e.g., spCas9, dCas9, nCas9, Cas9-SpRY, and Cas12a) and can have distinct characteristics, including for example recognition of distinct PAMs and PAM positions.

In various embodiments, a catalytically impaired nuclease domain generates a single-stranded nick in the non-deaminated DNA strand, inducing cells to repair the non-deaminated strand using the deaminated strand as a template. To provide one example, nCas9 can create a nick in target DNA by cutting a single strand, reducing the likelihood of detrimental indel formation as compared to methods that require a double-strand break.

Particular embodiments utilize a nuclease-inactive Cas9 (dCas9) as the catalytically disabled nuclease. However, any nuclease of the CRISPR system (many of which are described above) can be disabled and used within a base editing system. In particular embodiments, a Cas9 domain with high fidelity is selected wherein the Cas9 domain displays decreased electrostatic interactions between the Cas9 domain and a sugar-phosphate backbone of a DNA, as compared to a wild-type Cas9 domain. In some embodiments, a Cas9 domain (e.g., a wild type Cas9 domain) includes one or more mutations that decrease the association between the Cas9 domain and a sugar-phosphate backbone of a DNA. Cas9 domains with high fidelity are known to those skilled in the art. For example, Cas9 domains with high fidelity have been described in Kleinstiver (2016 Nature 529:490-495) and Slaymaker (2015 Science 351:84-88).

Other DNA binding nucleases can also be used in a base editing enzyme. For example, base-editing systems can utilize zinc finger nucleases (ZFNs) (see, e.g., Urnov 2010 Nat Rev Genet. 11 (9): 636-46) and transcription activator like effector nucleases (TALENs) (see, e.g., Joung 2013 Nat Rev Mol Cell Biol. 14 (1): 49-55). For additional information regarding DNA-binding nucleases, see, e.g., US 2018/0312825.

In various embodiments, a base editing enzyme includes a DNA glycosylase inhibitor. A DNA glycosylase inhibitor can override natural DNA repair mechanisms that might otherwise repair the intended base editing. A DNA glycosylase inhibitor can be a uracil DNA glycosylase inhibitor protein (UGI). One exemplary UGI is described in Wang (1991 Gene 99:31-37). In particular embodiments, a base editing enzyme can include one or more DNA glycosylase inhibitor domains (e.g., UGI domains). In various embodiments, base editing enzymes that include more than one DNA glycosylase inhibitor domain (e.g., UGI domain) can generate fewer indels and/or deaminate target nucleic acids more efficiently than base editing enzymes that includes one DNA glycosylase inhibitor domain (e.g., UGI domain) and/or no DNA glycosylase inhibitor domains (e.g., UGI domains). For example, in particular embodiments, dCas9 or a Cas9 nickase can be fused to a cytidine deaminase domain and the dCas9 or Cas9 nickase can be fused to one or more UGI domains.

In particular embodiments, a deaminase domain is associated with the N-terminus of a catalytically disabled nuclease. In particular embodiments, a deaminase domain is associated with the N-terminus of a catalytically disabled nuclease. In certain embodiments, one or more glycosylase inhibitors (e.g., UGI domain) can be associated with the C-terminus of a catalytically disabled nuclease.

Components of base editors can be fused directly (e.g., by direct covalent bond) or via linkers. For example, the catalytically disabled nuclease can be fused via a linker to the deaminase enzyme and/or a glycosylase inhibitor. Multiple glycosylase inhibitors can also be fused via linkers. As will be understood by one of ordinary skill in the art, linkers can be used to link any peptides or portions thereof.

Exemplary linkers include polymeric linkers (e.g., polyethylene, polyethylene glycol, polyamide, polyester); amino acid linkers; carbon-nitrogen bond amide linkers; cyclic or acyclic, substituted or unsubstituted, branched or unbranched aliphatic or heteroaliphatic linkers; monomeric, dimeric, or polymeric aminoalkanoic acid linkers; aminoalkanoic acid (e.g., glycine, ethanoic acid, alanine, B-alanine, 3-aminopropanoic acid, 4-aminobutanoic acid, 5-pentanoic acid) linkers; monomeric, dimeric, or polymeric aminohexanoic acid (Ahx) linkers; carbocyclic moiety (e.g., cyclopentane, cyclohexane) linkers; aryl or heteroaryl moiety linkers; and phenyl ring linkers.

Linkers can also include functionalized moieties to facilitate attachment of a nucleophile (e.g., thiol, amino) from a peptide to the linker. Any electrophile may be used as part of the linker. Exemplary electrophiles include activated esters, activated amides, Michael acceptors, alkyl halides, aryl halides, acyl halides, and isothiocyanates.

In particular embodiments, linkers range from 4-100 amino acids in length. In particular embodiments, linkers are 4 amino acids, 9 amino acids, 14 amino acids, 16 amino acids, 32 amino acids, or 100 amino acids.

Various base editing enzymes are known in the art. Examples of base editing enzymes include BE1 (APOBEC1-16 amino acid (aa) linker-Sp dCas9 (D10A, H840A) (see, e.g., Komor 2016 Nature 533:420-424)), BE2 (APOBEC1-16aa linker-Sp dCas9 (D10A, H840A)-4aa linker-UGI (see, e.g., Komor 2016 Nature 533:420-424)), BE3 (APOBEC1-16aa linker-SpnCas9 (D10A)-4aa linker-UGI (see, e.g., Komor 2016 Nature 533:420-424)), HF-BE2 (rAPOBEC1-HF2 nCas9-UGI), HF-BE3 (APOBEC1-16aa linker-HF nCas9 (D10A)-4aa linker-UGI (see, e.g., Rees 2017 Nat. Commun. 8:15790)), BE4 (rAPOBEC1-Sp nCas9-UGI-UGI), BE4max (APOBEC1-32aa linker-Sp nCas9 (D10A)-9aa linker-UGI-9aa linker-UGI (see, e.g., Koblan 2018 Nat. Biotechnol 36 (9): 843-846 and/or Komor 2017 Sci. Adv. 3 (8): eaao4774)), BE4-GAM (Gam-16aa linker-APOBEC1-32aa linker-Sp nCas9 (D10A)-9aa linker-UGI-9aa linker-UGI (see, e.g., Komor 2017 Sci. Adv. 3 (8): eaao4774)), YE1-BE3 (APOBEC1 (W90Y, R126E)-16aa linker-Sp nCas9 (D10A)-4aa linker-UGI (see, e.g., Kim 2017 Nat. Biotechnol. 35:475-480)), EE-BE3 (APOBEC1 (R126E, R132E)-16aa linker-Sp nCas9 (D10A)-4aa linker-UGI (see, e.g., Kim 2017 Nat. Biotechnol. 35:475-480)), YE2-BE3 (APOBEC1 (W90Y, R132E)-16aa linker-Sp nCas9 (D10A)-4aa linker-UGI (see, e.g., Kim 2017 Nat. Biotechnol. 35:475-480)), YEE-BE3 (APOBEC1 (W90Y, R126E, R132E)-16aa linker-Sp nCas9 (D10A)-4aa linker-UGI (see, e.g., Kim 2017 Nat. Biotechnol. 35:475-480)), VQR-BE3 (APOBEC1-16aa linker-Sp VQR nCas9 (D10A)-4aa linker-UGI (see, e.g., Kim 2017 Nat. Biotechnol. 35:475-480)), EQR-BE3 (rAPOBEC1-EQR SpnCas9-UGI), VRER-BE3 (APOBEC1-16aa linker-Sp VRER nCas9 (D10A)-4aa linker-UGI (see, e.g., Kim 2017 Nat. Biotechnol. 35:475-480)), Sa-BE3 (APOBEC1-16aa linker-Sa nCas9 (D10A)-4aa linker-UGI (see, e.g., Kim 2017 Nat. Biotechnol. 35:475-480)), SA-BE4 (APOBEC1-32aa linker-Sa nCas9 (D10A)-9aa linker-UGI-9aa linker-UGI (see, e.g., Komor 2017 Sci. Adv. 3 (8): eaao4774)), SaBE4-Gam (Gam-16aa linker-APOBEC1-32aa linker-Sa nCas9 (D10A)-9aa linker-UGI-9aa linker-UGI (see, e.g., Komor 2017 Sci. Adv. 3 (8): eaao4774)), SaKKH-BE3 (APOBEC1-16aa linker-Sa KKH nCas9 (D10A)-4aa linker-UGI (see, e.g., Kim 2017 Nat. Biotechnol. 35:475-480)), FNLS-BE3 (rAPOBEC1-Sp nCas9-UGI), RA-BE3 (rAPOBEC1 (RA)-Sp nCas9-UGI), Cas12a-BE (APOBEC1-16aa linker-dCas12a-14aa linker-UGI (see, e.g., Li 2018 Nat. Biotechnol. 36:324-327)), Target-AID (Sp nCas9 (D10A)-100aa linker-CDA1-9aa linker-UGI (see, e.g., Nishida 2016 Science 353 (6305): aaf8729)), Target-AID-NG (Sp nCas9 (D10A)-NG-100aa linker-CDA1-9aa linker-UGI (see, e.g., Nishimasu 2018 Science 361 (6408): 1259-1262)), xBE3 (APOBEC1-16aa linker-xCas9 (D10A)-4aa linker-UGI (see, e.g., Hu 2018 Nature 556:57-63)), eA3A-BE3 (APOBEC3A (N37G)-16aa linker-Sp nCas9 (D10A)-4aa linker-UGI (see, e.g., Gehrke 2018 Nat. Biotechnol. 36 (10): 977-982)), A3A-BE3 (hAPOBEC3A-16aa linker-Sp nCas9 (D10A)-4aa linker-UGI (see, e.g., Wang 2018 Nat. Biotechnol. 36:946-949)), eA3A-HF1-BE3-2xUGI (APOBEC3A-HF1 Sp nCas9-UGI-UGI), eA3A-HypaBE3-2xUGI (APOBEC3A-Hypa Sp nCas9-UGI-UGI), hA3A-BE3 (hAPOBEC3A-Sp nCas9-UGI), hA3B-BE3 (hAPOBEC3B-Sp nCas9-UGI), hA3G-BE3 (hAPOBEC3G-Sp nCas9-UGI), hAID-BE3 (hAPOBEC3A-Sp nCas9-UGI), SaCas9-BE3 (rAPOBEC1-SanCas9-UGI), xCas9-BE3 (rAPOBEC1-xnCas9-UGI), ScCas9-BE3 (rAPOBEC1-ScnCas9-UGI), SniperCas9-BE3 (rAPOBEC1-SnipernCas9-UGI), iSpyMac-BE3 (rAPOBEC1-iSpyMacnCas9-UGI), CRISPR-X (Sp dCas9-MS2-hAID), TAM (Sp dCas9-hAID (P182X)), AncBE4-Max (rAPOBEC1-Sp nCas9-UGI-UGI), ABE7.8/9/10 (ecTadA-ecTadA*-Sp nCas9), xCas9-ABE7.10 (ecTadA-ecTadA*-nxCas9), VQR-ABE (ecTadA-ecTadA*-Sp VQR nCas9), Sa (KKH)-ABE ecTadA-ecTadA*-Sa KKH nCas9), ABEmax (ecTadA-ecTadA*-Sp nCas9), ABE7.10max (ecTadA-ecTadA*-SpnCas9), ABE8e) ecTadA-ecTadA*-SpnCas9), ABE8e-V106W, PE1 (dSpCas9-MMLV-RT), PE2 (dSpCas9-MMLV-RT), PE3 (nSpCas9-MMLV-RT), and BE-PLUS (10X GCN4-Sp nCas9 (D10A)/ScFv-rAPOBEC1-UGI (see, e.g., Jiang 2018 Cell Res. 28 (8): 855-861)). For additional examples of BE complexes, including adenine deaminase base editors, see, e.g., Rees 2018 Nat. Rev Genet. 19 (12): 770-788 and/or Kantor 2020 Int. J. Mol. Sci. 21 (17): 6240.

Various base editors are “dual base editors” that can edit both adenine and cytosine. Dual base editor enzymes can be fusion polypeptides that include a cytosine deaminase domain and an adenine deaminase domain. For instance, a dual base editor known as Target-ACEmax includes a codon-optimized fusion of the cytosine deaminase PmCDA1, the adenosine deaminase TadA, and a Cas9 nickase (Target-ACEmax) (see, e.g., Sakata 2020 Nature Biotechnology, 38 (7), 865-869). Other exemplary dual base editors include SPACE (synchronous programmable adenine and cytosine editor). The SPACE editing enzyme is a fusion polypeptide that includes both miniABEmax-V82G and Target-AID editing domains together with a Cas9 (SpCas9-D10A) nickase domain (see, e.g., Grünewald 2020 Nat. Biotechnol. 38:861-864). A dual base editor known as A&C-BEmax includes a fusion of both cytidine and adenosine deaminase domains with a Cas9 nickase domain (see, e.g., Zhang 2020 Nat. Biotechnol. 38:856-860).

A base editing system can include a guide RNA (gRNA) that includes at least a fragment that base pairs with a complementary target nucleic acid (e.g., at least 80% identity between the fragment and the complement of the target nucleic acid, e.g., 80%, 85%, 90%, 91%, 92%, 93%, 94%, 95%, 96%, 97%, 98%, 99%, or 100% identity), wherein the fragment can be 10 to 40 nucleotides in length (e.g., equal to or about 10, 15, 16, 17, 18, 19, 20, 21, 22, 23, 24, 25, 30, 35 or 40 nucleotides in length, e.g., 17-24 or 17-20 nucleotides in length), e.g., where the target sequence is upstream of an appropriate PAM site. In various embodiments, a fragment of a gRNA that is complementary to a target nucleic acid sequence is positioned at the 5′ end of a gRNA or is 5′ relative to one or more other fragments of the gRNA. In various embodiments, a gRNA includes a sequence that forms a stemloop structure and binds with and/or recruits the catalytically impaired nuclease domain of a base editing enzyme. A gRNA that includes both a fragment that base pairs with a complementary target nucleic acid sequence and a fragment that forms a stemloop structure and binds with and/or recruits the catalytically impaired nuclease domain of a base editing enzyme can be referred to as a single guide RNA (sgRNA). The fragments of sgRNA can be associated via a linker fragment.

A guide RNA (e.g., an sgRNA) is thought to randomly interrogate nucleic acids until it encounters a nucleic acid that is sufficiently complementary to the 5′ fragment. Upon binding of a gRNA to a DNA nucleic acid target present in double-stranded DNA, base pairing between the gRNA and target nucleic acid strand causes displacement of a small segment of single-stranded DNA. In various embodiments, the gRNA recruits the catalytically impaired nuclease domain. Nucleotides of the displaced single-stranded DNA can be modified by the deaminase enzyme. The resultant base pair can then be repaired by cellular mismatch repair machinery to a new base pair, or alternatively in some instances reverted by base excision repair mediated by uracil glycosylase. In various embodiments, a glycosylase inhibitor (e.g., UGI) reduces the occurrence of reversion.

The present disclosure includes base editing enzymes and systems engineered to increase the editing window of base editing. For example, the present disclosure includes circularly permuted base editors, described for example in Huang 2020 Nature Biotechnology, 37 (6), 626-631, which is incorporated herein with respect to base editing enzymes, base editing systems, and editing windows thereof. Circularly permuted base editing enzymes and systems can be characterized by an increased range of target bases that can be modified within the protospacer up to and including, for example, at least 5, 6, 7, 8, or 9 nucleotides. For example, certain base editing systems including Cas9 variants, including cytosine and four adenine base editing enzymes, can deaminated nucleotides in a window expanded from about 4-5 nucleotides to up about 8-9 nucleotides, optionally with reduced byproduct formation.

Base editing enzymes and systems can also target and/or modify RNA molecules. One advantage of using RNA editing systems is that there is no permanent change in the genome. RNA base editors achieve analogous changes using components that base modify RNA. For example, adenosine deaminase can modify transcribed mRNA, replacing adenosine with inosine at a target site. In mammals, the most prevalent post-transcription RNA editing case is catalyzed by the adenosine deaminase enzymes (ADARs). ADAR proteins are a highly conserved family of proteins that include a single deaminase domain (DD) and one or more double-stranded RNA (dsRNA)-binding domains ADARs (e.g., ADAR 1 or ADAR2) bind to dsRNA and catalyzes adenosine to inosine (A-to-I), which is read as guanosine by cellular translational machinery. ADAR1 and ADAR2 domains have been demonstrated to achieve RNA editing, e.g., in HSCs (see, e.g., Harter 2009 Nat. Immunol. 10 (1): 109-115). A number of catalytically inactive Cas proteins have also been used to target RNA molecules, including Cas9, Cas13a, Cas13b, and Cas13d.

REPAIR (RNA editing for programmable adenosine to inosine replacement) is an RNA base editing system that includes catalytically inactive Cas13 protein and the deaminase activity of ADAR2. Cas13 generally includes two HEPN (higher eukaryotes and prokaryotes nucleotide-binding) domains, which contribute to RNA-targeted nucleolytic activity. Mutations of HEPNs abolish RNA cleavage activity while maintaining RNA targeting activity, which has been used to create an RNA base editing enzyme (e.g., REPAIR) (see, e.g., Cox 2017 Science 358:1019-1027). dCas13-ADAR2pp includes catalytically inactive dCas13 variant with RNA deaminase ADAR2 (E488Q), and can execute RNA editing for programmable A-to-I (G) replacement. RNA Editing for Specific C-to-U Exchange (RESCUE) was later developed (see, e.g., Abudayyeh 2019 Science 365:382-386). gRNAs for mRNA editing can include, e.g., a fragment complementary to a target RNA and an ADAR-recruiting fragment, such that site-directed RNA editing is achieved by recruiting ADAR to a complementary target nucleic acid. RNA-guided RNA-targeting CRISPR nuclease C2C2 (later named as Cas13a) from Leptotrichia shahii was illustrated (Abudayyeh 2016 Science 353: aaf5573).

Other examples of RNA editing systems that include ADARs can include removing the endogenous RNA-targeting domains (dsRBMS) from human adenosine deaminase and replacing them with an antisense RNA oligonucleotide to produce a recombinant enzyme that can be directed to edit a selected RNA target. In particular embodiments, an ADAR2 deaminase domain is fused with an RNA-binding protein, and the sequence bound by the RNA-binding protein is associated with an antisense RNA guide oligonucleotide. In various embodiments, the RNA-binding protein is derived from λ-phage N protein-boxB RNA interaction, which normally regulates antitermination during transcription of λ-phage mRNAs. AN peptide mediates binding of the N protein, is only 22 amino acids long, and the boxB RNA hairpin that it recognizes is only 17 nucleotides long and they can bind with nanomolar affinity. Thus, in various embodiments, AN peptide can be fused to the deaminase domain of human ADAR2 (λN-DD). In various embodiments, a mutant ADAR2DD (E488Q) can be used as the deaminase domain. In various embodiments, an editing enzyme can include an ADAR deaminase domain and 2 or more AN domains (e.g., 2, 3, 4, 5, or 6 λN domains). Examples of such editing enzymes and systems are described, e.g., in Montiel-Gonzalez 2013 PNAS 110 (45): 18285-18290 and Montiel-Gonzalez 2016 Nuc. Acids. Res. 44 (2): e157, each of which is incorporated herein by reference with respect to editing systems.

Other examples of editing systems that include ADARs can include leveraging endogenous ADAR for programmable editing of RNA (LEAPER) editing system that employs short engineered ADAR-recruiting RNAs (arRNAs) to recruit native ADAR1 or ADAR2 deaminase enzymes to change a specific adenosine to inosine. For example, in certain particular embodiments, an ADAR protein or its catalytic domain can be fused with a AN peptide. In certain embodiments, an ADAR protein or its catalytic domain can be fused with a AN peptide and a SNAP-tag or a Cas protein (e.g., dCas13b). A gRNA can recruit the editing enzyme to the specific site. Further description of LEAPER editing systems can be found in Qu 2019 Nat. Biotech. 1059-1069, which is incorporated herein by reference with respect to LEAPER editing systems and

Base editing systems can cause point mutations without producing double-strand breaks. Base editing systems can cause point mutations without producing undesired insertions and deletions (indels). For example, a base editing system can cause indels in less than 10%, 9%, 8%, 7%, 6%, 5.5%, 5%, 4.5%, 4%, 3.5%, 3%, 2.5%, 2%, 1.5%, 1%, 0.5%, or 0.1% of edited cells or editing events.

Those of skill in the art will appreciate that a base editing gRNA (e.g., sgRNA) or other targeting elements to generate a selected nucleic acid sequence modification in a target nucleic acid can be readily designed and implemented, e.g., based on available sequence information.

In various embodiments, base editing system can be used for editing of MGMT-encoding nucleic acids to produce a modified MGMT-encoding nucleic acid that encodes an inhibitor-resistant MGMT polypeptide. In some embodiments, base editing systems can be used to produce a modified MGMT-encoding nucleic acid that encodes an inhibitor-resistant MGMT polypeptide that includes at least one amino acid mutation selected from L33F, L33P, M134V, R135G, R135K, R135T, N137D, P140F, G156P, S159F, S159P, S159W, G160E, G160K, and G160P. In some embodiments, base editing systems can be used to produce a modified MGMT-encoding nucleic acid that encodes an inhibitor-resistant MGMT polypeptide that includes at least one amino acid mutation selected from P140R, G156A, Y158H, and G160A. The present disclosure includes base editing systems that include a plurality of sgRNAs (e.g., two or more, e.g., two, three, four, or five) sgRNAs. In certain embodiments, two or more sgRNAs are used to target multiple sequences of a single nucleic acid to produce an inhibitor-resistant MGMT mutation (e.g., an inhibitor-resistant MGMT mutation of Table 1 or Table 2). For instance, in some embodiments, an inhibitor-resistant MGMT mutation includes two or more sequence modifications at positions that are separated by a plurality of nucleotides of genomic DNA (e.g., 10, 20, 30, 40, 50, 60, 70, 80, 90, 100, or more nucleotides of genomic DNA). In certain such instances, a first sgRNA can target a base editing enzyme to a first target sequence of a nucleic acid for a first modification and a second (or further subsequent) sgRNA can target the base editing enzyme to a second target sequence of the nucleic acid for the second (or further subsequent) modification(s), which modifications together produce the inhibitor-resistant MGMT mutation and/or produce a modified nucleic acid encoding an inhibitor-resistant MGMT polypeptide. Exemplary of certain such inhibitor-resistant MGMT mutations are inhibitor-resistant MGMT mutations that include modification of codons encoding each of amino acid 138, amino acid 139, and amino acid 140 of MGMT.

Base editing systems do not require double-stranded DNA breaks. Base editing systems do not require a donor fragment or template. Base editing systems provide precise control of the site at which the editing system modifies a target nucleic acid. Base editing systems can be multiplexed to achieve editing of multiple targets using a single editing enzyme, optionally including therapeutic targets.

### Prime Editing Enzymes and Systems for Modification of Nucleic Acids Encoding MGMT

The present disclosure includes editing systems that utilize a reverse transcriptase (e.g., a prime editing system) for editing of nucleic acid targets, including in various embodiments modification an MGMT-encoding nucleic acid to produce a nucleic acid encoding inhibitor-resistant MGMT. In various embodiments, an editing agent includes an editing enzyme that includes a reverse transcriptase domain. A reverse transcriptase is an enzyme that can synthesize a DNA molecule from an RNA template. A reverse transcriptase generally produces a DNA molecule that is complementary to the RNA template.

In particular embodiments, an editing enzyme includes an AMV reverse transcriptase, MLV reverse transcriptase, HIV-1 reverse transcriptase, or bacterial reverse transcriptase. Certain embodiments utilize an MLV reverse transcriptase domain. Reverse transcriptases of the present disclosure can have wild type amino acid sequences or engineered amino acid sequences.

Examples of reverse transcriptase enzymes include AMV reverse transcriptases (e.g., wild type AMV reverse transcriptase (RNase H plus activity), eAMV™ (engineered; RNase Hplus activity) or ThermoScript™ (engineered; reduce RNAase H activity)), MLV reverse transcriptases (e.g., wild type M-MLV reverse transcriptase, GoScript™, or MultiScribe™ (RNase H plus activity), AccuScript Hi-Fi (engineered, RNase H minus (3′-5′ exonuclease activity), Affinity Script (engineered; E69K/E302R/W313F/L435G/N454K; unspecified RNase H activity), ArrayScript™ (engineered; unspecified RNase H activity), BioScript™ (engineered; reduced RNase H activity), CycleScript™ (engineered), EnzScript™ (engineered; RNase H minus), EpiScript™ (engineered; RNase H minus), Expand™ reverse transcriptase (engineered; RNase H reduced), FIREScript (engineered; RNase H plus), GrandScript (engineered; RNase H plus), iScript™ (engineered; RNase H plus), Maxima™ RT (engineered; RNase H plus and minus), MonsterScript™ (engineered; RNase H minus), PrimeScript™ (engineered; RNase H minus), PrimeScript™ II (engineered; RNase H minus), PrimeScript™ III (engineered; RNase H minus), PrimeScript™ IV (engineered: RNase H minus), ProtoScript® (Engineered; RNase H plus), ProtoScript® II (engineered; RNase H reduced), qScript (engineered; RNase H plus), RevertAid™ (engineered; RNase H plus and minus), ReverTra Ace® (engineered; RNase H minus), RevertUp II™ (engineered; RNase H minus), Rocketscript™ (engineered; RNase H plus and minus), Script (engineered; RNase H minus), SMART® (engineered), SMARTScribe™ (engineered; unspecified RNase H activity), SuperScript™ II (engineered; 524G/D583N/E562Q; RNase H reduced), SuperScript™ III (engineered; 204R/V223H/T306K/F309N/D524G/D583N/E562Q; RNase H reduced), SuperScript™ IV (engineered; RNase H reduced), or Transcriptor reverse transcriptase (engineered; RNase H plus)), an HIV-1 reverse transcriptase (e.g., HIV-1 RT (wild type of group M subtype B; RNase H plus), Biotools high retrotranscriptase (engineered group O variant (K65R/V751); RNase H plus), or Sunscript® (engineered group O variants with changes K358R/A359G/S360A; RNase H plus and minus)), a bacterial group II intron reverse transcriptase (e.g., Marathon RT (wild type (Eubacterium rectale); lacks RNase H domain) or TGIRT®-III RT (wild type (Geobacillus stearothermophilus); lacks RNase H domain), a bacterial DNA polymerase (e.g., BcaBEST polymerase (engineered (Bacillus caldotenax DNA polymerase without 5′-3′ and 3′-5′ exonuclease activity); lacks RNase H domain), Bst 3.0 DNA polymerase (G. stearothermophilus DNA polymerase I, large fragment; lacks 5′-3′ and 3′-5′ exonuclease activity; lacks RNase H domain), RapiDxFire™ reverse transcriptase (lacks RNase H domain), Volcano2G DNA polymerase (engineered Thermus aquaticus DNA polymerase; lacks RNase H domain), or Volcano3G DNA polymerase (engineered T. aquaticus DNA polymerase; lacks RNase H domain)), SOLIScript (engineered; RNase H reduced), Omniscript® (heterodimeric RT; RNase H plus), and SensiScript® (heterodimeric RT; RNase H plus).

In various embodiments, a reverse transcriptase is a retrovirus reverse transcriptase. In various embodiments, a reverse transcriptase is a murine leukemia virus (MLV) reverse transcriptase (RT) (e.g., an engineered MLV RT). In various embodiments, a reverse transcriptase is a bacterial group II intron RT.

In various embodiments, an editing enzyme or system includes a reverse transcriptase associated with a DNA binding domain such as a catalytically impaired nuclease domain. In various embodiments, the DNA binding domain can localize the reverse transcriptase to a target nucleic acid in which one or more nucleotides are substituted, inserted, and/or deleted.

Prime editing enzymes and systems are exemplary of editing enzymes and systems that include reverse transcriptase. A prime editing enzyme includes a reverse transcriptase fused to a DNA binding domain that is a catalytically impaired nuclease domain (e.g., a nickase, e.g., a nickase that nicks a single strand, e.g., a non-edited strand). DNA binding domains of prime editing enzymes can be RNA guided DNA binding domains, in that an RNA guide can direct the DNA binding domain to a target nucleic acid sequence. Catalytically impaired nuclease domains of a prime editing enzyme can bind nucleic acids and can localize the reverse transcriptase enzyme to a target nucleic acid in which one or more nucleotides are substituted, inserted, and/or deleted by the prime editing system.

Any nuclease of the CRISPR system can be engineered to produce a catalytically impaired nuclease domain (e.g., a nickase) and used within a prime editing enzyme or system. Exemplary Cas nucleases include Cas1, Cas1B, Cas2, Cas3, Cas4, Cas5, Cas6, Cas7, Cas8, Cas9 (also known as Csn1 or Csx12), Cas10, Cas12, CasX, CasY, C2c3, C2c2 and C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, and variants thereof. Numerous forms and variants of Cas nucleases are known in the art (e.g., spCas9, dCas9, nCas9, Cas9-SpRY, and Cas12a) and can have distinct characteristics, including for example recognition of distinct PAMs and PAM positions.

Other DNA binding nucleases can also be used in a prime editing enzyme. For example, prime editing systems can utilize zinc finger nucleases (ZFNs) (see, e.g., Urnov 2010 Nat Rev Genet. 11 (9): 636-46) and transcription activator like effector nucleases (TALENs) (see, e.g., Joung 2013 Nat Rev Mol Cell Biol. 14 (1): 49-55). For additional information regarding DNA-binding nucleases, see, e.g., US 2018/0312825.

In various embodiments, a prime editing system includes a prime editing gRNA (pegRNA) that specifies a target nucleic acid sequence and also specifies the sequence modification that the prime editing system introduces. The pegRNA includes a sequence complimentary to the target nucleic acid and recruits the prime editing enzyme to the target nucleic acid. A pegRNA includes, from 5′ to 3′: (a) a fragment that base pairs with a complementary target nucleic acid sequence (e.g., at least 80% identity between the fragment and the complement of the target nucleic acid, e.g., 80%, 85%, 90%, 91%, 92%, 93%, 94%, 95%, 96%, 97%, 98%, 99%, or 100% identity) (sometimes referred to as a “spacer”), wherein the fragment can be 10 to 40 nucleotides in length (e.g., equal to or about 10, 15, 16, 17, 18, 19, 20, 21, 22, 23, 24, 25, 30, 35 or 40 nucleotides in length, e.g., 17-24 or 17-20 nucleotides in length); (b) a sequence that forms a stemloop structure and binds with and/or recruits the catalytically impaired nuclease domain of a prime editing enzyme; (c) a fragment that includes a sequence that includes one or more modifications (e.g., one or more substitutions, insertions, and/or deletions) relative to the target nucleic acid sequence (sometimes referred to as a “template sequence”), and is complementary (excepting modifications) to the same target nucleic acid strand as (d); and (d) a fragment that includes a sequence complimentary to a target sequence (sometimes referred to as a “binding region” or “primer binding site” (PBS)), e.g., where the target sequence is upstream of an appropriate PAM site. In various embodiments, a PBS can be 5 to 20 nucleotides, e.g., 8 to 15 nucleotides in length. In various embodiments, a template sequence can be 10 to 20 nucleotides in length, or longer. Because pegRNAs include components characteristic of sgRNAs, they are sometimes described as extended sgRNAs. Any two fragments of a pegRNA can be, independently, associated directly or via a linker fragment.

A catalytically impaired nuclease domain of a prime editing enzyme can nick a target nucleic acid that includes an appropriate PAM to expose a 3′ flap and a 5′ flap. After nicking of the target nucleic acid, the released 3′ flap can hybridize to the PBS of the pegRNA, priming reverse transcription of the template fragment of the pegRNA that includes a modification of the target sequence, directly introducing the modification into the target nucleic acid to the 3′ flap. The product of reverse transcription, an edited 3′ flap that is “redundant” with the 5′ flap sequence produced by the nick (which includes the original, unedited sequence of the target nucleic acid), can then compete with the original and redundant 5′ flap sequence for reincorporation into the DNA duplex. Although the perfectly complimentary 5′ would likely be thermodynamically favored for hybridization to the non-edited strand, the 5′ flap is preferentially degraded by cellular endonucleases that are ubiquitous during lagging-strand DNA synthesis. After 5′ flap excision and ligation of the edited strand, permanent installation of the edit occurs through DNA repair of the non-edited that relies on the editing strand as a template. DNA repair of the non-edited strand can be promoted by contact with a secondary sgRNA that directs nicking of the non-edited strand. This additional nick stimulates re-synthesis of the non-edited strand using the edited strand as a template, resulting in a fully edited duplex. Prime editing systems can introduce any of one or more of the 12 types of point mutations (all possible nucleotide transitions and transversions), as well as insertions and/or deletions.

In various embodiments, a prime editing system is engineered to disrupt a PAM site of a target nucleic acid. Disruption of a PAM site of a target nucleic acid can reduce the probability of repeated editing of the particular target nucleic acid. In various embodiments, disruption of a PAM site in edited target nucleic acids can increase the efficiency of prime editing and/or gene therapy that includes prime editing.

Exemplary prime editing systems include PE1, PE2, and PE3. Each of these prime editing enzymes include a mutant Streptococcus pyogenes Cas9 nickase domain (H840A mutant) and a Moloney murine leukemia virus (M-MLV) reverse transcriptase (e.g., engineered to include D200N/T306K/W313F/T330P/L603W). PE1 includes a pegRNA and a prime editing enzyme that includes a Cas9 H840A nickase and wild type MLV RT. The Cas9 nickase acts only on the strand to be edited by the RT. PE2 includes pegRNA and a prime editing enzyme that includes a Cas9 H840A nickase and engineered MLV RT (D200N/T306K/W313F/T330P/L603W) demonstrated to improve editing efficiency. PE3 includes the same prime editing enzyme as PE2 (as well as a pegRNA) but further includes an sgRNA that targets the non-edited strand for nicking 14-116 nucleotides away from the site of the pegRNA-induced nick (PE3), where cellular mismatch repair pathways can fix the information introduced in the edited strand. Compared with PE2, the PE3b strategy demonstrate increased editing efficiency and lower levels of indel formation. A variant of the PE3 system called PE3b uses a nicking sgRNA that targets only the edited sequence, resulting in decreased levels of indel products by preventing nicking of the non-edited DNA strand until the other strand has been converted to the edited sequence.

Those of skill in the art will appreciate that a pegRNA or other targeting elements to generate a selected nucleic acid sequence modification in a target nucleic acid can be readily designed and implemented, e.g., based on available sequence information. Various tools for designing pegRNAs are available. For example, pegFinder is a web-based tool for pegRNA design (see, e.g., Chow 2020 Nat. Biomed. Eng. doi: 10.1038/s41551-020-00622-8). Another example of a web-based tool for pegRNA design is PrimeDesign (see, e.g., Hsu 2020 bioRxiv doi: 10.1101/2020.05.04.077750).

In various embodiments, prime editing systems can be used for editing of MGMT-encoding nucleic acids to produce a modified MGMT-encoding nucleic acid that encodes an inhibitor-resistant MGMT polypeptide. In some embodiments, prime editing systems can be used to produce a modified MGMT-encoding nucleic acid that encodes an inhibitor-resistant MGMT polypeptide that includes at least one amino acid mutation selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P. In some embodiments, prime editing systems can be used to produce a modified MGMT-encoding nucleic acid that encodes an inhibitor-resistant MGMT polypeptide that includes at least one amino acid mutation selected from L33K, L33R, L33W, L33Y, M134F, M134W, M134Y, R135L, N137F, N137P, P138K, P140E, P140H, G156I, G156V, Y158M, Y158W, S159I, S159L, S159T, S159Y, G160D, and G160H. In some embodiments, prime editing systems can be used to produce a modified MGMT-encoding nucleic acid that encodes an inhibitor-resistant MGMT polypeptide that includes at least one amino acid mutation selected from P140R, P140Q, G156A, Y158F, Y158H, G160A, G160S, and A170S. In some embodiments, prime editing systems can be used to produce a modified MGMT-encoding nucleic acid that encodes an inhibitor-resistant MGMT polypeptide that includes the amino acid mutation P140K.

The present disclosure includes that a prime editing system can include a plurality of pegRNAs (e.g., two or more, e.g., two, three, four, or five pegRNAs). In certain embodiments, two or more pegRNAs are used to target multiple sequences of a single nucleic acid to produce an inhibitor-resistant MGMT mutation (e.g., an inhibitor-resistant MGMT mutation of Table 1 or Table 2). For instance, in some embodiments, an inhibitor-resistant MGMT mutation includes two or more sequence modifications at positions that are separated by a plurality of nucleotides of genomic DNA (e.g., 10, 20, 30, 40, 50, 60, 70, 80, 90, 100, or more nucleotides of genomic DNA). In certain such instances, a first pegRNA can target a prime editing enzyme to a first target sequence of a nucleic acid for a first modification and a second (or further subsequent) pegRNA can target the prime editing enzyme to a second target sequence of the nucleic acid for the second (or further subsequent) modification(s), which modifications together produce the inhibitor-resistant MGMT mutation and/or produce a modified nucleic acid encoding an inhibitor-resistant MGMT polypeptide. Exemplary of certain such inhibitor-resistant MGMT mutations are inhibitor-resistant MGMT mutations that include modification of codons encoding each of amino acid 138, amino acid 139, and amino acid 140 of MGMT.

Prime editing systems do not require double-stranded DNA breaks. Prime editing systems provide precise control of the site at which the editing system modifies a target nucleic acid. Prime editing systems can be multiplexed to achieve editing of multiple targets using a single editing enzyme, optionally including therapeutic targets.

### CRISPR Enzymes and Systems for Modification of Nucleic Acids Encoding MGMT

The present disclosure includes CRISPR editing systems for editing of nucleic acid targets, including in various embodiments modification an MGMT-encoding nucleic acid to produce a nucleic acid encoding inhibitor-resistant MGMT. In various embodiments, an editing agent includes an editing enzyme that is an endonuclease. CRISPR (Clustered Regularly Interspaced Short Palindromic Repeats) enzymes and systems are exemplary of editing enzymes and systems that include an endonuclease. CRISPR editing systems include an engineered endonuclease (e.g., engineered Cas (CRISPR-associated) endonuclease) and are based, in part, on discoveries relating to immune responses of bacteria and archaea. When a virus or plasmid invades a bacterium, segments of the invader's DNA are converted into CRISPR RNAs (crRNA) by the bacteria's “immune” response. The crRNA then associates, through a region of partial complementarity, with another type of RNA called tracrRNA to guide a Cas nuclease to a region homologous to the crRNA in the target DNA called a “protospacer.” The Cas nuclease cleaves the DNA to generate blunt ends at the double-strand break at sites specified by a complementary sequence contained within the crRNA transcript.

Endonuclease domains of CRISPR editing enzymes can e RNA guided DNA binding domains, in that an RNA guide can direct the DNA binding domain to a target nucleic acid sequence. An endonuclease associated with an sgRNA can randomly interrogate DNA in a cell until contacting a nucleic acid including an appropriate protospacer adjacent motif (PAM) (e.g., in proximity to a target sequence). Upon recognition of the PAM sequence, the endonuclease unwinds the DNA, allowing the associated sgRNA to contact and/or hybridize with the exposed DNA strand (the protospacer). If the DNA sequence matches the sgRNA target sequence, the endonuclease catalytic domains (e.g., HNH and RuvC) can cleave both strands of the target DNA, generating a double-strand break, which can be repaired by NHEJ or HDR mechanisms.

Exemplary Cas nucleases include Cas1, Cas1B, Cas2, Cas3, Cas4, Cas5, Cas6, Cas7, Cas8, Cas9 (also known as Csn1 and Csx12), Cas10, Cas12, CasX, CasY, C2c3, C2c2 and C2c1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Cpf1, Csb1, Csb2, Csb3, Csx17, Csx14, Csx10, Csx16, CsaX, Csx3, Csx1, Csx15, Csf1, Csf2, Csf3, Csf4, and variants thereof. Cas endonucleases have been classified into at least three types (type I, type II, and type III), and at least 10 subtypes. Type II Cas nucleases include Cas1, Cas2, Csn2, and Cas9.

To provide one particular example, Cas9 refers to an RNA-guided double-stranded DNA-binding nuclease protein or nickase protein. Cas9 enzyme, in some embodiments, includes one or more catalytic domains of a Cas9 protein derived from bacteria such as Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. Wild-type Cas9 nuclease has two functional domains, e.g., RuvC and HNH, that cut different DNA strands. Cas9 can induce double-strand breaks in genomic DNA (target DNA) when both functional domains are active. In some embodiments, the Cas9 is a fusion protein, e.g., a fusion of two nickases and/or where the two catalytic domains are derived from different bacterial species. In various embodiments, variants of the Cas9 nuclease include a single inactive catalytic domain, such as a RuvC or HNH enzyme or a nickase. A Cas9 nickase has only one active functional domain and, in some embodiments, cuts only one strand of the target DNA, thereby creating a single-strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include N854A and N863 A. A double-strand break is introduced using a Cas9 nickase if at least two DNA-targeting RNAs that target opposite DNA strands are used. A double-nicked induced double-strand break can be repaired by HDR.

A CRISPR editing system can include a gRNA that includes at least a fragment that base pairs with a complementary target nucleic acid (sometimes referred to as a crRNA) and a fragment that associates with an endonuclease (sometimes referred to as a tracrRNA). In particular embodiments, a gRNA including a fragment that base pairs with a complementary target nucleic acid and a fragment that associates with an endonuclease can be referred to as an sgRNA.

In various embodiments, a gRNA is expressed from a nucleic acid and is not modified after expression from the nucleic acid. In various embodiments, a gRNA can be modified. In particular embodiments, a gRNA can include one or more modifications (e.g., a base modification, a backbone modification). Modified backbones may include those that retain a phosphorus atom in the backbone and those that do not have a phosphorus atom in the backbone. Suitable modified backbones containing a phosphorus atom may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable targeting elements having inverted polarity can include a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

Those of skill in the art will appreciate that a CRISPR editing gRNA (e.g., sgRNA) or other targeting elements to generate a selected nucleic acid sequence modification in a target nucleic acid can be readily designed and implemented, e.g., based on available sequence information. Thus, a CRISPR/Cas system can be engineered to create a double-strand break at a desired target in a genome of a cell, and can harness the cell's endogenous mechanisms to repair the induced break by HDR.

In particular embodiments, a double-strand break can be repaired by homology-directed repair (HDR) when a donor template is present. The donor template can be used as a template by cellular (e.g., endogenous) repair enzymes, incorporating sequence based on the donor template into the nucleic acid being repaired. The donor template can be a foreign nucleic acid such as a nucleic acid introduced in conjunction with delivery of an editing system of the present disclosure. A donor template can be used to introduce small changes (e.g., a mutation of one or a few nucleotides) or larger modifications (e.g., addition to the repaired nucleic acid of an entire transgene).

CRISPR editing is among the editing systems and methods that can be advantageously applied for editing of endogenous nucleic acids of target cells as set forth in the present disclosure. Like other editing systems disclosed herein, CRISPR permits rapid, efficient, and robust editing of nucleic acid targets.

### Zinc Finger Nucleases for Modification of Nucleic Acids Encoding MGMT

The present disclosure includes Zinc Finger Nuclease editing systems for editing of nucleic acid targets, including in various embodiments modification an MGMT-encoding nucleic acid to produce a nucleic acid encoding inhibitor-resistant MGMT. Zinc finger nucleases (ZFNs) are artificial restriction enzymes made by associating a sequence-targeted zinc-finger DNA-binding units with a nuclease domain (e.g., Fok1 nuclease domain) in a fusion protein. Each ZFN includes a nuclease domain (e.g., the cleavage domain of FokI) linked to an array of three to six zinc fingers zinc fingers (ZFs). For example, a ZFN can include several CyszHisz ZFs in which each unit includes about 30 amino acids and specifically binds about 3 nucleotides. The ZFs provide a ZFN with the ability to bind a particular nucleic acid sequence. Because the FokI cleavage domain must dimerize to cut DNA, a monomer is not active, and cleavage does not occur at single binding sites. Thus, for example, ZFNs including three ZFs that together bind a 9-bp target function as ZFN dimers that specifically bind 18 bp of DNA per cleavage site. In some embodiments, ZFNs can include up to six ZFs per ZFN.

Cleave of a target nucleic acid by ZFNs induces cellular repair processes that can mediate modification of the nucleic acid. ZFN-induced double-strand breaks can lead to both targeted modification and targeted gene replacement. For example, if a ZFN-induced cleavage is resolved by non-homologous end joining, this can result in small deletions or insertions, which can lead to gene knockout. If a ZFN-induced cleavage is resolved by a homology-based process in the presence of a provided donor nucleic acid, small changes (e.g., one or a few nucleotides) or more (e.g., up to and including entire transgenes) can be introduced into the target nucleic acid.

### TALENs for Modification of Nucleic Acids Encoding MGMT

The present disclosure includes Transcription Activator-Like Effector Nuclease (TALEN) editing systems for editing of nucleic acid targets, including in various embodiments modification an MGMT-encoding nucleic acid to produce a nucleic acid encoding inhibitor-resistant MGMT. Various editing enzymes and systems can include a transcription activator-like (TAL) effector DNA binding domain and an endonuclease enzyme. An editing enzyme including a TAL effector DNA binding domain and an endonuclease can be referred to as a TALEN.

TAL effector DNA binding domains includes a plurality of monomers, each of which monomers binds one nucleotide in the target nucleic acid sequence. Each monomer includes 34 amino acids. In each monomer, positions 12 and 13 (referred to as the repeat variable diresidue, RVD) are highly variable and contribute to specific recognition of different nucleotides. The final monomer of a TAL effector DNA binding domain, which binds the nucleotide at the 3′-end of the recognition site, can be only 20 amino acids in length and therefore is sometimes referred to as a half-repeat. RVD sequences can be degenerate, as certain RVD combinations can bind to two or more nucleotides, e.g., with distinct efficiency. For example, RVDs include Asn and Ile (NI), Asn and Gly (NG), Asn and Asn (NN), and His and Asp (HD), which bind A, T, G, and C nucleotides, respectively.

In various embodiments, a TAL effector DNA binding domain is isolated from Xanthomonas spp. In various embodiments, a TALEN includes an endonuclease domain (e.g., a FokI domain), e.g., C-terminal to the TAL effector DNA binding domain.

TALENs work as pairs, the two members having target binding site on opposite DNA strands of the target nucleic acid sequence, with the targets separated by a small fragment (e.g., 12-25 bp) that can be referred to as a spacer sequence. Once a pair of TALENs have bound their target sites, the endonuclease (e.g., FokI) domains dimerize and cause a double-strand break in a spacer sequence. Non-homologous end joining (NHEJ) to resolve a DSB directly ligates DNA from either side of the double-strand break where there is very little or no sequence overlap for annealing. This repair mechanism can cause indels (insertion or deletion), or chromosomal rearrangement, which can disrupt genes at that target nucleic acid sequence. Alternatively, DNA can be introduced into a genome through NHEJ in the presence of exogenous double-stranded DNA fragments. Homology directed repair can also introduce foreign DNA at the DSB as the transfected double-stranded sequences are used as templates for the repair enzymes

### Applications

In various embodiments, inhibitor-resistant MGMT sequences that include one or more mutations provided here can be used in cells. For example, in various embodiments, one or more cells can be engineered to include and/or express a nucleic acid sequence that encodes an inhibitor-resistant MGMT sequence that includes one or more mutations provided herein. As is discussed herein, certain such engineered cells can have a selective advantage in a cell population, tissue, organ, organism, or other system that also includes cells that do not include inhibitor-resistant, e.g., upon exposure to an MGMT inhibitor and/or an alkylating agent. The present disclosure therefore includes the production and use of such engineered cells, which production can be by any means known in the art and which use can be a use provided herein, e.g., for gene therapy. Production of engineered cells that include and/or express a nucleic acid sequence that encodes an inhibitor-resistant MGMT can be, for example, by transduction or transfection of cells with a nucleic acid that encodes and/or expresses inhibitor-resistant MGMT, or by editing of an endogenous MGMT-encoding nucleic acid sequence to produce a nucleic acid sequence that encodes inhibitor-resistant MGMT.

In vivo, in vitro, and or ex vivo modification of endogenous MGMT-encoding nucleic acids to encode an inhibitor-resistant MGMT can selectively protect MGMT-modified cells (i.e., cells including the modified nucleic acids) from a selection regimen including an MGMT inhibitor. In various embodiments, modified cells are selectively protected against the effects of MGMT inhibitors, and cells that do not encode and/or express inhibitor-resistant MGMT (“non-MGMT-modified cells” or “non-modified cells”) are vulnerable to the effects of MGMT inhibitors. In the presence of the selection regimen including an MGMT inhibitor, cells selectively protected by modification to encode an inhibitor-resistant MGMT can survive and/or proliferate at a greater rate and/or frequency than cells that are not selectively protected. For at least this reason, modification of endogenous MGMT-encoding nucleic acids as disclosed herein is useful in increasing the in vivo, in vitro, and or ex vivo prevalence of modified cells (e.g., therapeutic cells) as compared to non-modified cells, which in various embodiments can improve therapeutic efficacy.

### Nucleic Acids Encoding Editing Systems

In various embodiments, methods of the present disclosure that include modification of endogenous MGMT-encoding nucleic acids of target cells can include delivery to a subject, system, or cell of an editing system disclosed herein. In various embodiments, methods of the present disclosure that include modification of endogenous MGMT-encoding nucleic acids of target cells can include delivery to a subject, system, or cell of a nucleic acid encoding an editing system disclosed herein

The present disclosure includes compositions including a nucleic acid that encodes an editing system disclosed herein (which nucleic acid can be referred to as an “editing nucleic acid”). A nucleic acid encoding an editing system of the present disclosure can include one or more fragments each encoding one or more components of the editing system (and/or a fragment encoding the editing enzyme) operably linked with regulatory sequences such as a promoter. In various embodiments, with respect to a nucleic acid that encodes an editing system of the present disclosure, the one or more fragments of the nucleic acid that encode the one or more components of the editing system can be referred to as an “MGMT editing payload.” A nucleic acid encoding an editing system can further include a “therapeutic payload.” A therapeutic payload can refer to one or more fragments of a nucleic acid that encode one or more agents that cause, elicit, or contribute to a desired pharmacological and/or physiological effect (e.g., treatment of a disease, disorder, or condition) not achieved by modification of endogenous MGMT-encoding nucleic acids alone.

For avoidance of doubt, an editing nucleic acid of the present disclosure refers to any nucleic acid that encodes an editing system and can further include additional sequences including, for example, other payloads and/or functional sequences that do not perform or contribute to gene editing. Thus, to provide one example, an editing nucleic acid can refer to a viral vector genome that includes an MGMT editing payload.

In various embodiments, an MGMT editing payload is present in a nucleic acid that includes a therapeutic payload, where the therapeutic payload includes one or more components of an editing system that causes, elicits, or contributes to a desired pharmacological and/or physiological effect by editing a target nucleic acid sequence (a “therapeutic editing payload”).

At least in part because editing systems disclosed herein can be engineered to cause a wide variety of nucleic acid changes, editing systems disclosed herein are capable of treating a wide variety of genetic diseases, disorders, and conditions. To provide just one example, editing systems of the present disclosure can be used to treat a disease, disorder, or condition caused by a point mutation in the genomic DNA of a subject. Examples of diseases, disorders, and conditions that can result from point mutations include, without limitation cystic fibrosis, sickle cell anemia, phenylketonuria, and Tay-Sachs. Those of skill in the art will appreciate, however, that therapeutic editing payloads can have many other types of targets.

As further examples of therapeutic editing targets, various therapeutic editing systems can target one or more nucleic acid sequences to cause increased expression of a globin polypeptide. In some embodiments, a therapeutic gene editing payload encodes one or more components of a therapeutic gene editing system engineered to modify a nucleic acid sequence that encodes γ-globin, e.g., to increase expression of γ-globin. The main fetal form of hemoglobin, hemoglobin F (HbF) is formed by pairing of γ-globin polypeptide subunits with α-globin polypeptide subunits. Human fetal γ-globin genes (HBG1 and HBG2; two highly homologous genes produced by evolutionary duplication) are ordinarily silenced around birth, while expression of adult β-globin gene expression (HBB and HBD) increases. Mutations that cause or permit persistent expression of fetal γ-globin throughout life can ameliorate phenotypes of β-globin deficiencies. Thus, reactivation of fetal γ-globin genes can be therapeutically beneficial, particularly in subjects with β-globin deficiency. A variety of mutations that cause increased expression of γ-globin are known in the art (see, e.g., Wienert, Trends in Genetics 34 (12): 927-940, 2018, which is incorporated herein by reference in its entirety and with respect to mutations that increase expression of γ-globin). Certain such mutations are found in the HBG1 promoter or HBG2 promoter.

In various embodiments, a therapeutic gene editing system that is designed to increase expression of γ-globin targets an HBG1/2 promoter and is designed to increase expression of γ-globin coding by modification and/or inactivation of a BCL11A repressor protein binding site. In various embodiments, a therapeutic gene editing system that is designed to increase expression of γ-globin targets the erythroid bcl11a enhancer and is designed to increase expression of γ-globin by modification and/or inactivation of the erythroid bcl11a enhancer to reduce BCL11A repressor protein expression in erythroid cells. In various embodiments, a therapeutic gene editing system that is designed to increase expression of γ-globin is targeted to cause a loss of function mutation in the gene encoding BCL11A.

In various embodiments, an MGMT editing payload is present in a nucleic acid that includes a therapeutic editing payload, where the MGMT editing system and therapeutic editing system are “multiplexed” in that the MGMT editing system and therapeutic editing system include and/or utilize the same editing enzyme encoded by the same nucleic acid fragment. Thus, in various embodiments, a nucleic acid encoding editing systems for both MGMT editing and therapeutic editing can encode a single editing enzyme that participates in and/or causes both the MGMT editing and the therapeutic editing. To provide one example, in various multiplexed embodiments, (i) an MGMT editing payload encodes an MGMT editing system that includes a base editing enzyme and a base editing gRNA, (ii) a therapeutic editing payload encodes a therapeutic base editing gRNA, and (iii) the MGMT editing and the therapeutic editing utilize (and/or the MGMT editing system and therapeutic editing system include) the same base editing enzyme. To provide another example, in various multiplexed embodiments, (i) an MGMT editing payload encodes an MGMT editing system that includes a prime editing enzyme and a pegRNA, (ii) a therapeutic editing payload encodes a therapeutic pegRNA, and (iii) the MGMT editing and the therapeutic editing utilize (and/or the MGMT editing system and therapeutic editing system include) the same prime editing enzyme. In various embodiments, one or more components of a multiplexed system can be operably linked to distinct regulatory elements (e.g., distinct promoters), operably linked to a single regulatory element (e.g., a single promoter), or each operably linked to separate copies of the same regulatory element (e.g., separate copies of the same promoter).

The present disclosure includes embodiments in which an MGMT editing payload is present in a nucleic acid that includes a therapeutic editing payload, where the MGMT editing system and therapeutic editing system are not multiplexed, in that the MGMT editing system and therapeutic editing system do not include and/or utilize any of the same editing system components (i.e., have no shared components, e.g., no shared editing enzyme). Thus, for example, a nucleic acid of the present disclosure can include an MGMT editing payload that encodes a base editing system and a therapeutic editing payload that encodes a prime editing system. To provide another example, an editing nucleic acid of the present disclosure can include an MGMT editing payload that encodes a prime editing system and a therapeutic editing payload that encodes a base editing system.

In various embodiments, an MGMT editing payload is present in a nucleic acid that also includes a therapeutic payload that does not encode an editing system. In various embodiments, an MGMT editing payload is present in a nucleic acid that also includes a sequence encoding an expression product that is not a component of an editing system, optionally wherein the expression product is a therapeutic agent (such as a therapeutic polypeptide or therapeutic RNA). Exemplary expression products include proteins, including without limitation replacement therapy proteins for treatment of diseases or conditions characterized by low expression or activity of a biologically active protein as compared to a reference level. Exemplary expression products include antibodies, CARs, and TCRs. Exemplary expression products include small RNAs.

Particular examples of therapeutic genes and/or expression products include y-globin, Factor VIII, γC, JAK3, IL7RA, RAG1, RAG2, DCLRE1C, PRKDC, LIG4, NHEJ1, CD3D, CD3E, CD3Z, CD3G, PTPRC, ZAP70, LCK, AK2, ADA, PNP, WHN, CHD7, ORAI1, STIM1, CORO1A, CIITA, RFXANK, RFX5, RFXAP, RMRP, DKC1, TERT, TINF2, DCLRE1B, SLC46A1, a FANC family gene (e.g., FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)), soluble CD40, CTLA, Fas L, an antibody (e.g., that specifically binds CD4, CD5, CD7, CD52, IL1, IL2, IL6, TNF, P53, PTPN22, or DRB1*1501/DQB1*0602), an antibody to TCR specifically present on autoreactive T cells, IL4, IL10, IL12, IL13, IL1Ra, sIL1RI, SIL1RII, STNFRI, STNFRII, globin family genes, WAS, phox, dystrophin, pyruvate kinase, CLN3, ABCD1, arylsulfatase A, SFTPB, SFTPC, NLX2.1, ABCA3, GATA1, ribosomal protein genes, TERT, TERC, DKC1, TINF2, CFTR, LRRK2, PARK2, PARK7, PINK1, SNCA, PSEN1, PSEN2, APP, SOD1, TDP43, FUS, ubiquilin 2, C9ORF72, and other therapeutic genes and/or expression products described herein.

A therapeutic gene and/or expression product can be selected to provide a therapeutically effective response against diseases related to red blood cells and clotting. In particular embodiments, the disease is a hemoglobinopathy like thalassemia, or a sickle cell disease/trait. The therapeutic gene and/or expression product may be, for example, a gene that induces or increases production of hemoglobin; induces or increases production of β-globin, γ-globin, or α-globin; or increases the availability of oxygen to cells in the body. The therapeutic gene and/or expression product may be, for example, HBB or CYBER3. Exemplary effective treatments may, for example, increase blood cell counts, improve blood cell function, or increase oxygenation of cells in patients. In another particular embodiment, the disease is hemophilia. The therapeutic gene may be, for example, a gene that increases the production of coagulation/clotting factor VIII or coagulation/clotting factor IX, causes the production of normal versions of coagulation factor VIII or coagulation factor IX, a gene that reduces the production of antibodies to coagulation/clotting factor VIII or coagulation/clotting factor IX, or a gene that causes the proper formation of blood clots. Exemplary therapeutic genes and/or expression products include F8 and F9. Exemplary effective treatments may, for example, increase or induce the production of coagulation/clotting factors VIII and IX; improve the functioning of coagulation/clotting factors VIII and IX, or reduce clotting time in subjects.

In various embodiments of the present disclosure, a therapeutic payload encodes a globin gene, wherein the globin protein encoded by the globin gene is selected from a γ-globin, a β-globin, and/or an α-globin. Globin genes of the present disclosure can include, e.g., one or more regulatory sequences such as a promoter operably linked to a nucleic acid sequence encoding a globin protein. As those of skill in the art will appreciate, each of γ-globin, β-globin, and/or α-globin is a component of fetal and/or adult hemoglobin and is therefore useful in various vectors disclosed herein.

In various embodiments, increasing expression of a globin protein can refer to any of one or more of (i) increasing the amount, concentration, or expression (e.g., transcription or translation of nucleic acids encoding) in a cell or system of globin protein having a particular sequence; (ii) increasing the amount, concentration, or expression (e.g., transcription or translation of nucleic acids encoding) in a cell or system of globin protein of a particular type (e.g., the total amount of all proteins that would be identified as γ-globin (or alternatively β-globin or α-globin) by those of skill in the art or as set forth in the present specification) without respect to the sequences of the proteins relative to each other; and/or (iii) expressing in a cell or system a heterologous globin protein, e.g., a globin protein not encoded by a host cell prior to gene therapy.

The following references describe particular exemplary sequences of functional globin genes. References 1˜4 relate to α-type globin sequences and references 4-12 relate to β-type globin sequences (including β and γ globin sequences), which sequences are hereby incorporated by reference: (1) GenBank Accession No. Z84721 (Mar. 19, 1997); (2) GenBank Accession No. NM_000517 (Oct. 31, 2000); (3) Hardison et al., J. Mol. Biol. (1991) 222 (2): 233-249; (4) A Syllabus of Human Hemoglobin Variants (1996), by Titus et al., published by The Sickle Cell Anemia Foundation in Augusta, Ga. (available online at globin.cse.psu.edu); (5) GenBank Accession No. J00179 (Aug. 26, 1993) or U01317.1; (6) Tagle et al., Genomics (1992) 13 (3): 741-760; (7) Grovsfeld et al., Cell (1987) 51 (6): 975-985; (8) Li et al., Blood (1999) 93 (7): 2208-2216; (9) Gorman et al., J. Biol. Chem. (2000) 275 (46): 35914-35919; (10) Slightom et al., Cell (1980) 21 (3): 627-638; (11) Fritsch et al., Cell (1980) 19 (4): 959-972; (12) Marotta et al., J. Biol. Chem. (1977) 252 (14): 5040-5053. For additional coding and non-coding regions of genes encoding globins see, for example, by Marotta et al., Prog. Nucleic Acid Res. Mol. Biol. 19, 165-175, 1976, Lawn et al., Cell 21 (3), 647-651, 1980, and Sadelain et al., PNAS.; 92:6728-6732, 1995. In some embodiments, a globin gene encodes a G16D gamma globin variant.

An exemplary amino acid sequence of hemoglobin subunit β is provided, for example, at NCBI Accession No. P68871. An exemplary amino acid sequence for β-globin is provided, for example, at NCBI Accession No. NP_000509.

Exemplary therapeutic genes and/or expression products also include checkpoint inhibitor reagents, chimeric antigen receptor molecules specific to one or more cancer antigens, and/or T-cell receptors specific to one or more cancer antigens.

As another example, a therapeutic gene can be selected to provide a therapeutically effective response against a lysosomal storage disorder. In particular embodiments, the lysosomal storage disorder is mucopolysaccharidosis (MPS), type I; MPS II or Hunter Syndrome; MPS III or Sanfilippo syndrome; MPS IV or Morquio syndrome; MPS V; MPS VI or Maroteaux-Lamy syndrome; MPS VII or sly syndrome; α-mannosidosis; β-mannosidosis; glycogen storage disease type I, also known as GSDI, von Gierke disease, or Tay-Sachs; Pompe disease; Gaucher disease; or Fabry disease. The therapeutic gene and/or expression product may, for example, be, encode, or induce expression of an enzyme, or that otherwise causes the degradation of mucopolysaccharides in lysosomes. Exemplary therapeutic genes and/or expression products include IDUA or iduronidase, IDS, GNS, HGSNAT, SGSH, NAGLU, GUSB, GALNS, GLB1, ARSB, and HYAL1. Exemplary effective genetic therapies for lysosomal storage disorders may, for example, encode or induce the production of enzymes responsible for the degradation of various substances in lysosomes; reduce, eliminate, prevent, or delay the swelling in various organs, including the head (exp. Macrosephaly), the liver, spleen, tongue, or vocal cords; reduce fluid in the brain; reduce heart valve abnormalities; prevent or dilate narrowing airways and prevent related upper respiratory conditions like infections and sleep apnea; reduce, eliminate, prevent, or delay the destruction of neurons, and/or the associated symptoms.

As another example, a therapeutic gene and/or expression product can be selected to provide a therapeutically effective response against a hyperproliferative disease. In particular embodiments, the hyperproliferative disease is cancer. The therapeutic gene and/or expression product may be, for example, a tumor suppressor gene, a gene that induces apoptosis, a gene encoding an enzyme, a gene encoding an antibody, or a gene encoding a hormone. Exemplary therapeutic genes and expression products include (in addition to those listed elsewhere herein) 101F6, 123F2 (RASSF1), 53BP2, abl, ABLI, ADP, aFGF, APC, ApoAI, ApoAIV, ApoE, ATM, BAI-1, BDNF, Beta*(BLU), bFGF, BLC1, BLC6, BRCA1, BRCA2, CBFA1, CBL, C-CAM, CNTF, COX-1, CSFIR, CTS-1, cytosine deaminase, DBCCR-1, DCC, Dp, DPC-4, E1A, E2F, EBRB2, erb, ERBA, ERBB, ETS1, ETS2, ETV6, Fab, FCC, FGF, FGR, FHIT, fms, FOX, FUS1, FYN, G-CSF, GDAIF, Gene 21 (NPRL2), Gene 26 (CACNA2D2), GM-CSF, GMF, gsp, HCR, HIC-1, HRAS, hst, IGF, IL-1, IL-2, IL-3, IL-5, IL-6, IL-7, IL-8, IL-9, IL-11, ING1, interferon α, interferon β, interferon γ, IRF-1, JUN, KRAS, LUCA-1 (HYAL1), LUCA-2 (HYAL2), LYN, MADH4, MADR2, MCC, mda7, MDM2, MEN-I, MEN-II, MLL, MMACI, MYB, MYC, MYCLI, MYCN, neu, NF-1, NF-2, NGF, NOEY1, NOEY2, NRAS, NT3, NT5, OVCA1, p16, p21, p27, p57, p73, p300, PGS, PIMI, PL6, PML, PTEN, raf, Rap1A, ras, Rb, RB1, RET, rks-3, ScFv, scFV ras, SEM A3, SRC, TALI, TCL3, TFPI, thrombospondin, thymidine kinase, TNF, TP53, trk, T-VEC, VEGF, VHL, WT1, WT-1, YES, and zac1. Exemplary effective genetic therapies may suppress or eliminate tumors, result in a decreased number of cancer cells, reduced tumor size, slow or eliminate tumor growth, or alleviate symptoms caused by tumors.

As another example, a therapeutic gene and/or expression product can be selected to provide a therapeutically effective response against an infectious disease. In particular embodiments, the infectious disease is human immunodeficiency virus (HIV). The therapeutic gene and/or expression product may be, for example, a gene rendering immune cells resistant to HIV infection, or which enables immune cells to effectively neutralize the virus via immune reconstruction, polymorphisms of genes encoding proteins expressed by immune cells, genes advantageous for fighting infection that are not expressed in the patient, genes encoding an infectious agent, receptor or coreceptor; a gene encoding ligands for receptors or coreceptors; viral and cellular genes essential for viral replication including; a gene encoding ribozymes, antisense RNA, small interfering RNA (siRNA) or decoy RNA to block the actions of certain transcription factors; a gene encoding dominant negative viral proteins, intracellular antibodies, intrakines and suicide genes. Exemplary therapeutic genes and expression products include α2β1; αvβ3; αvβ5; αvβ63; BOB/GPR15; Bonzo/STRL-33/TYMSTR; CCR2; CCR3; CCR5; CCR8; CD4; CD46; CD55; CXCR4; aminopeptidase-N; HHV-7; ICAM; ICAM-1; PRR2/HveB; HveA; a-dystroglycan; LDLR/a2MR/LRP; PVR; PRR1/HveC; and laminin receptor. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

The present disclosure includes payloads that can include sequences that encode any of a variety of binding domains. Sequences that encode binding domains can encode, for example, antibodies, chimeric antigen receptors, TCRs, or other binding polypeptides.

Antibodies and antibody fragments are exemplary of binding domains. The term “antibody” can refer to a polypeptide that includes one or more canonical immunoglobulin sequence elements sufficient to confer specific binding to a particular antigen (e.g., a heavy chain variable domain, a light chain variable domain, and/or one or more CDRs). Thus, the term antibody includes, without limitation, human antibodies, non-human antibodies, synthetic and/or engineered antibodies, fragments thereof, and agents including the same. Antibodies can be naturally occurring immunoglobulins (e.g., generated by an organism reacting to an antigen). Synthetic, non-naturally occurring, or engineered antibodies can be produced by recombinant engineering, chemical synthesis, or other artificial systems or methodologies known to those of skill in the art.

As is well known in the art, typical human immunoglobulins are approximately 150 kD tetrameric agents that include two identical heavy (H) chain polypeptides (about 50 kD each) and two identical light (L) chain polypeptides (about 25 kD each) that associate with each other to form a structure commonly referred to as a “Y-shaped” structure. Typically, each heavy chain includes a heavy chain variable domain (VH) and a heavy chain constant domain (CH). The heavy chain constant domain includes three CH domains: CH1, CH2 and CH3. A short region, known as the “switch”, connects the heavy chain variable and constant regions. The “hinge” connects CH2 and CH3 domains to the rest of the immunoglobulin. Each light chain includes a light chain variable domain (VL) and a light chain constant domain (CL), separated from one another by another “switch.” Each variable domain contains three hypervariable loops known as “complement determining regions” (CDR1, CDR2, and CDR3) and four somewhat invariant “framework” regions (FR1, FR2, FR3, and FR4). In each VH and VL, the three CDRs and four FRs are arranged from amino-terminus to carboxy-terminus in the following order: FR1, CDR1, FR2, CDR2, FR3, CDR3, and FR4. The variable regions of a heavy and/or a light chain are typically understood to provide a binding moiety that can interact with an antigen. Constant domains can mediate binding of an antibody to various immune system cells (e.g., effector cells and/or cells that mediate cytotoxicity), receptors, and elements of the complement system. Heavy and light chains are linked to one another by a single disulfide bond, and two other disulfide bonds connect the heavy chain hinge regions to one another, so that the dimers are connected to one another and the tetramer is formed. When natural immunoglobulins fold, the FR regions form the beta sheets that provide the structural framework for the domains, and the CDR loop regions from both the heavy and light chains are brought together in three-dimensional space so that they create a single hypervariable antigen binding site located at the tip of the Y structure.

In some embodiments, an antibody is polyclonal, monoclonal, monospecific, or multispecific antibodies (including bispecific antibodies). In some embodiments, an antibody includes at least one light chain monomer or dimer, at least one heavy chain monomer or dimer, at least one heavy chain-light chain dimer, or a tetramer that includes two heavy chain monomers and two light chain monomers. Moreover, the term “antibody” can include (unless otherwise stated or clear from context) any art-known constructs or formats utilizing antibody structural and/or functional features including without limitation intrabodies, domain antibodies, antibody mimetics, Zybodies®, Fab fragments, Fab′ fragments, F(ab′)2 fragments, Fd′ fragments, Fd fragments, isolated CDRs or sets thereof, single chain antibodies, single-chain Fvs (scFvs), disulfide-linked Fvs (sdFv), polypeptide-Fc fusions, single domain antibodies (e.g., shark single domain antibodies such as IgNAR or fragments thereof), cameloid antibodies, camelized antibodies, masked antibodies (e.g., Probodies®), affybodies, anti-idiotypic (anti-Id) antibodies (including, e.g., anti-anti-Id antibodies), Small Modular ImmunoPharmaceuticals (“SMIPs™”), single chain or Tandem diabodies (TandAb®), VHHs, Anticalins®, Nanobodies® minibodies, BiTE®s, ankyrin repeat proteins or DARPINs®, Avimers®, DARTs, TCR-like antibodies, Adnectins®, Affilins®, Trans-bodies®, Affibodies®, TrimerX®, MicroProteins, Fynomers®, Centyrins®, and KALBITOR®s, CARs, engineered TCRs, and antigen-binding fragments of any of the above.

In various embodiments, an antibody includes one or more structural elements recognized by those skilled in the art as a complementarity determining region (CDR) or variable domain. In some embodiments, an antibody can be a covalently modified (“conjugated”) antibody (e.g., an antibody that includes a polypeptide including one or more canonical immunoglobulin sequence elements sufficient to confer specific binding to a particular antigen, where the polypeptide is covalently linked with one or more of a therapeutic agent, a detectable moiety, another polypeptide, a glycan, or a polyethylene glycol molecule). In some embodiments, antibody sequence elements are humanized, primatized, chimeric, etc., as is known in the art.

An antibody including a heavy chain constant domain can be, without limitation, an antibody of any known class, including but not limited to, IgA, secretory IgA, IgG, IgE, and IgM, based on heavy chain constant domain amino acid sequence (e.g., alpha (a), delta (8), epsilon (¿), gamma (γ) and mu (u)). IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. As used herein, a “light chain” can be of a distinct type, e.g., kappa (κ) or lambda (λ), based on the amino acid sequence of the light chain constant domain. In some embodiments, an antibody has constant region sequences that are characteristic of mouse, rabbit, primate, or human immunoglobulins. Naturally-produced immunoglobulins are glycosylated, typically on the CH2 domain. As is known in the art, affinity and/or other binding attributes of Fc regions for Fc receptors can be modulated through glycosylation or other modification. In some embodiments, an antibody may lack a covalent modification (e.g., attachment of a glycan) that it would have if produced naturally. In some embodiments, antibodies produced and/or utilized in accordance with the present invention include glycosylated Fc domains, including Fc domains with modified or engineered such glycosylation.

The term “antibody fragment” can refer to a portion of an antibody or antibody agent as described herein, and typically refers to a portion that includes an antigen-binding portion or variable region thereof. An antibody fragment can be produced by any means. For example, in some embodiments, an antibody fragment can be enzymatically or chemically produced by fragmentation of an intact antibody or antibody agent. Alternatively, in some embodiments, an antibody fragment can be recombinantly produced (i.e., by expression of an engineered nucleic acid sequence. In some embodiments, an antibody fragment can be wholly or partially synthetically produced. In some embodiments, an antibody fragment (particularly an antigen-binding antibody fragment) can have a length of at least about 50, 60, 70, 80, 90, 100, 110, 120, 130, 140, 150, 160, 170, 180, 190 amino acids or more, in some embodiments at least about 200 amino acids.

In some instances, it is beneficial for the binding domain to be derived from the same species it will ultimately be used in. For example, for use in humans, it may be beneficial for the antigen binding domain to include a human antibody, humanized antibody, or a fragment or engineered form thereof. Antibodies from human origin or humanized antibodies have lowered or no immunogenicity in humans and have a lower number of non-immunogenic epitopes compared to non-human antibodies. Antibodies and their engineered fragments will generally be selected to have a reduced level or no antigenicity in human subjects.

In various embodiments, a payload can encode a binding agent that is a checkpoint inhibitor such as an antibody that specifically binds an immune checkpoint protein. A number of immune checkpoint inhibitors are known. Immune checkpoint inhibitors can include peptides, antibodies, nucleic acid molecules and small molecules. Examples of immune checkpoints include PD-1, PD-L1, lymphocyte activation gene-3 (LAG-3), and T cell immunoglobulin and mucin domain-containing molecule 3 (TIM-3).

The present disclosure further includes antibodies and other binding domains that bind CD4, CD5, CD7, CD52, etc.: antibodies; antibodies to IL1, IL2, IL6; an antibody to TCR specifically present on autoreactive T cells; IL4; IL10; IL12; IL13; IL1Ra; sIL1RI; sIL1RII; antibodies to TNF; ABCA3; ABCD1; ADA; AK2; APP; arginase; arylsulfatase A; A1AT; CD3D; CD3E; CD3G; CD3Z; CFTR; CHD7; chimeric antigen receptor (CAR); CIITA; CLN3; complement factor, CORO1A; CTLA; C1 inhibitor; C9ORF72; DCLRE1B; DCLRE1C; decoy receptors; DKC1; DRB1*1501/DQB1*0602; dystrophin; enzymes: Factor VIII, FANC family genes (FancA, FancB, FancC, FancD1 (BRCA2), FancD2, FancE, FancF, FancG, FancI, FancJ (BRIP1), FancL, FancM, FancN (PALB2), FancO (RAD51C), FancP (SLX4), FancQ (ERCC4), FancR (RAD51), FancS (BRCA1), FancT (UBE2T), FancU (XRCC2), FancV (MAD2L2), and FancW (RFWD3)); Fas L; FUS; GATA1; globin family genes (i.e., γ-globin); F8; glutaminase; HBA1; HBA2; HBB; IL7RA; JAK3; LCK; LIG4; LRRK2; NHEJ1; NLX2.1; neutralizing antibodies; ORAI1; PARK2; PARK7; phox; PINK1; PNP; PRKDC; PSEN1; PSEN2; PTPN22; PTPRC; P53; pyruvate kinase; RAG1; RAG2; RFXANK; RFXAP; RFX5; RMRP; ribosomal protein genes; SFTPB; SFTPC; SOD1; soluble CD40; STIM1; sTNFRI; sTNFRII; SLC46A1; SNCA; TDP43; TERT; TERC; TINF2; ubiquilin 2; WAS; WHN; ZAP70; γC; and other therapeutic genes and/or expression products described herein. In various embodiments, an antibody can be a multispecific antibody.

HSCs can be engineered to encode and/or express chimeric antigen receptor (CAR) constructs. CARs can include several distinct subcomponents that can cause cells to recognize and kill target cells such as cancer cells. The subcomponents include at least an extracellular component and an intracellular component. In various embodiments, the subcomponents can include at least an extracellular component, a transmembrane domain, and an intracellular component.

An extracellular CAR component can include a binding domain that specifically binds a marker that is preferentially present on the surface of unwanted cells. When the binding domain binds such markers, the intracellular component directs a cell to destroy the bound cancer cell. The binding domain is typically a single-chain variable fragment (scFv) derived from a monoclonal antibody (mAb), but it can be based on other formats which include an antibody-like antigen binding site.

Intracellular CAR components provide activation signals based on the inclusion of an effector domain. First generation CARs utilized the cytoplasmic region of CD35 as an effector domain. Second generation CARs utilized CD32 in combination with cluster of differentiation 28 (CD28) or 4-1BB (CD137), while third generation CARs have utilized CD35 in combination with CD28 and 4-1BB within intracellular effector domains.

Intracellular or otherwise the cytoplasmic signaling components of a CAR are responsible for activation of the cell in which the CAR is expressed. The term “intracellular signaling components” or “intracellular components” is thus meant to include any portion of the intracellular domain sufficient to transduce an activation signal. Intracellular components of expressed CAR can include effector domains. An effector domain is an intracellular portion of a fusion protein or receptor that can directly or indirectly promote a biological or physiological response in a cell when receiving the appropriate signal. In certain embodiments, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other embodiments, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular embodiments, an effector domain can include an intracellular signaling component including a T cell receptor and a co-stimulatory domain which can include the cytoplasmic sequence from a co-receptor or co-stimulatory molecule.

An effector domain can include one, two, three or more receptor signaling domains, intracellular signaling components (e.g., cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTa, PTCH2, OX40, ROR2, Ryk, SLAMF1, Slp76, TCRα, TCRβ, TRIM, Wnt, Zap70, or any combination thereof. In particular embodiments, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, or NKp46.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCERIG), FcγRIIa, FcRβ (Fcε Rib), DAP10, and DAP12. In particular embodiments, variants of CD35 retain at least one, two, three, or all ITAM regions.

In particular embodiments, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3L chain, and/or co-receptors that act in concert to initiate signal transduction following binding domain engagement.

A co-stimulatory domain is domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART cells in vitro and augments human T cell persistence and anti-cancer activity in vivo (Song et al., Blood. 2012; 119 (3): 696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CDlld, ITGAE, CD103, ITGAL, CDlla, ITGAM, CDI 1b, ITGAX, CDllc, ITGBl, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, and CD19a.

In particular embodiments, the amino acid sequence of the intracellular signaling component includes a variant of CD3} and a portion of the 4-1BB intracellular signaling component.

In particular embodiments, the intracellular signaling component includes (i) all or a portion of the signaling domain of CD34, (ii) all or a portion of the signaling domain of 4-1BB, or (iii) all or a portion of the signaling domain of CD33 and 4-1BB.

Intracellular components may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR): or cytokine receptors (IL1R, IL2R, IL7R, or IL15R).

CAR generally also include one or more linker sequences that are used for a variety of purposes within the molecule. For example, a transmembrane domain can be used to link the extracellular component of the CAR to the intracellular component. A flexible linker sequence often referred to as a spacer region that is membrane-proximal to the binding domain can be used to create additional distance between a binding domain and the cellular membrane. This can be beneficial to reduce steric hindrance to binding based on proximity to the membrane. A common spacer region used for this purpose is the IgG4 linker. More compact spacers or longer spacers can be used, depending on the targeted cell marker. Other potential CAR subcomponents are described in more detail elsewhere herein.

Transmembrane domains within a CAR molecule, often serve to connect the extracellular component and intracellular component through the cell membrane. The transmembrane domain can anchor the expressed molecule in the modified cell's membrane.

The transmembrane domain can be derived either from a natural and/or a synthetic source. When the source is natural, the transmembrane domain can be derived from any membrane-bound or transmembrane protein. Transmembrane domains can include at least the transmembrane region(s) of the a, B or & chain of a T-cell receptor, CD28, CD27, CD3 epsilon, CD45, CD4, CD5, CD8, CD9, CD16, CD22; CD33, CD37, CD64, CD80, CD86, CD134, CD137 and CD154. In particular embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CD11a, ITGAM, CD11b, ITGAX, CD11c, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C. In particular embodiments, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge.

TCRs refer to naturally occurring T cell receptors. Payloads of the present disclosure can encode a TCR or a CAR/TCR hybrids that includes an element of a TCR and an element of a CAR. For example, a CAR/TCR hybrid could have a naturally occurring TCR binding domain with an effector domain that the TCR binding domain is not naturally associated with. A CAR/TCR hybrid could have a mutated TCR binding domain and an ITAM signaling domain. A CAR/TCR hybrid could have a naturally occurring TCR with an inserted non-naturally occurring spacer region or transmembrane domain.

Small RNAs are short, non-coding RNA molecules that play a role in regulating gene expression. In particular embodiments, small RNAs are less than 200 nucleotides in length. In particular embodiments, small RNAs are less than 100 nucleotides in length. In particular embodiments, small RNAs are less than 50, 45, 40, 35, 30, 25, or 20 nucleotides in length. In particular embodiments, small RNAs are less than 20 nucleotides in length. In various embodiments, a small RNA has a length having a lower bound of 5, 10, 15, 20, 25, or 30 nucleotides and an upper bound of 20, 25, 30, 35, 40, 45, 50, 75, or 100 nucleotides. Small RNAs include but are not limited to microRNAs (miRNAs, Piwi-interacting RNAs (piRNAs), small interfering RNAs (siRNAs), small nucleolar RNAs (snoRNAs), tRNA-derived small RNAs (tsRNAs) small rDNA-derived RNAs (srRNAs), and small nuclear RNAs. Additional classes of small RNAs continue to be discovered.

In particular embodiments, interfering RNA molecules that are homologous to a target mRNA or to which the interfering RNA can hybridize can lead to degradation of the target mRNA molecule or reduced translation of the target mRNA, a process referred to as RNA interference (RNAi) (Carthew, Curr. Opin. Cell. Biol. 13:244-248, 2001). RNAi occurs in cells naturally to remove foreign RNAs (e.g., viral RNAs). In some instances, natural RNAi proceeds via fragments cleaved from free double-strand RNA (dsRNA) which direct the degradative mechanism to other similar RNA sequences. Alternatively, RNAi can be manufactured, for example, to silence the expression of target genes. Exemplary RNAi molecules include small hairpin RNA (shRNA, also referred to as short hairpin RNA) and small interfering RNA (siRNA).

Without limiting the disclosure, and without being bound by theory, RNA interference in nature and/or in some embodiments is typically a two-step process. In the first step, the initiation step, input dsRNA is digested into 21-23 nucleotide (nt) siRNA, probably by the action of Dicer, a member of the ribonuclease (RNase) III family of dsRNA-specific ribonucleases, which processes (cleaves) dsRNA (introduced directly or via a transgene or a virus) in an ATP-dependent manner. Successive cleavage events degrade the RNA to 19-21 base pair (bp) duplexes (siRNA), each with 2-nucleotide 3′ overhangs.

In a second step, an effector step, the siRNA duplexes bind to a nuclease complex to form the RNA-induced silencing complex (RISC). An ATP-dependent unwinding of the siRNA duplex is required for activation of the RISC. The active RISC then targets the homologous transcript by base pairing interactions and typically cleaves the mRNA into 12 nucleotide fragments from the 3′ terminus of the siRNA. Research indicates that each RISC contains a single siRNA and an RNase.

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC.

ShRNAs are single-stranded polynucleotides with a hairpin loop structure. The single-stranded polynucleotide has a loop segment linking the 3′ end of one strand in the double-stranded region and the 5′ end of the other strand in the double-stranded region. The double-stranded region is formed from a first sequence that is hybridizable to a target sequence, such as a polynucleotide encoding transgene, and a second sequence that is complementary to the first sequence, thus the first and second sequence form a double-stranded region to which the linking sequence connects the ends of to form the hairpin loop structure. The first sequence can be hybridizable to any portion of a polynucleotide encoding transgene. The double-stranded stem domain of the shRNA can include a restriction endonuclease site.

Transcription of shRNAs is initiated at a polymerase III (Pol III) promoter and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of 21-23 nucleotides.

The stem-loop structure of shRNAs can have optional nucleotide overhangs, such as 2-bp overhangs, for example, 3′ UU overhangs. While there may be variation, stems typically range from 15 to 49, 15 to 35, 19 to 35, 21 to 31 bp, or 21 to 29 bp, and the loops can range from 4 to 30 bp, for example, 4 to 23 bp. In particular embodiments, shRNA sequences include 45-65 bp; 50-60 bp; or 51, 52, 53, 54, 55, 56, 57, 58, or 59 bp. In particular embodiments, shRNA sequences include 52 or 55 bp. In particular embodiments, siRNAs have 15-25 bp. In particular embodiments, siRNAs have 16, 17, 18, 19, 20, 21, 22, 23, or 24 bp. In particular embodiments, siRNAs have 19 bp. The skilled artisan will appreciate, however, that siRNAs having a length of less than 16 nucleotides or greater than 24 nucleotides can also function to mediate RNAi. Longer RNAi agents have been demonstrated to elicit an interferon or Protein kinase R (PKR) response in certain mammalian cells which may be undesirable. Preferably the RNAi agents do not elicit a PKR response (i.e., are of a sufficiently short length). However, longer RNAi agents may be useful, for example, in situations where the PKR response has been downregulated or dampened by alternative means.

In certain illustrative embodiments, the present disclosure includes an adenoviral vector payload that encodes an shRNA targeted to the gene encoding BCL11A, where the shRNA causes decreased translation of BCL11A.

In various embodiments, an editing nucleic acid of the present disclosure includes a fragment that integrates (or is engineered to integrate) into genomic DNA of a target or recipient subject, cell, or system. In various embodiments, a payload can include a nucleic acid sequence engineered for integration into a host cell genome (an “integrating payload”), e.g., by recombination or transposition. In various embodiments, an editing nucleic acid of the present disclosure includes a fragment that does not integrate (and/or is not engineered to integrate) into genomic DNA of a target or recipient subject, cell, or system (a “non-integrating payload”). In various embodiments, a payload can include a nucleic acid sequence that is not engineered for integration into a host cell genome in that it is not flanked by sequences for recombination with genomic DNA and/or transposition into genomic DNA (e.g., is not flanked by transposon inverted repeats). In various embodiments, a payload this is not specifically associated with and/or engineered to include sequences that cause integration into genomic DNA can be referred to as a non-integrating payload, e.g., when present in a vector or vector nucleic acid sequence (e.g., a viral vector genome) that is not characterized by an ability to integrate into genomic DNA. An editing nucleic acid of the present disclosure can include an “integrating” portion that is engineered to integrate into genomic DNA of a target or recipient subject, cell, or system and a “non-integrating” portion that is not engineered to integrate into genomic DNA of a target or recipient subject, cell, or system.

In various embodiments, an MGMT editing payload is present in a nucleic acid that includes at least one therapeutic payload (e.g., a therapeutic payload that does not encode an editing system), where the therapeutic payload is integrating and the MGMT editing payload is non-integrating. In various embodiments, an integrating payload can encode one or more expression products for which permanent, long-term, or lineage-enduring expression is desired. For example, a gene that provides an expression product that counteracts a deficiency of endogenous cells (e.g., an enzyme deficiency and/or a deficiency resulting from a genomic mutation and/or a deficiency associated with a disease, disorder, or condition) can be included in an integration portion of an editing nucleic acid of the present disclosure. In various embodiments, an editing enzyme or editing system of the present disclosure is encoded by a non-integrating portion of an editing nucleic acid of the present disclosure, e.g., to minimize the level and/or duration of expression and/or activity of an encoded agent, and where applicable genotoxicity, fitness cost, or other undesired effects resulting therefrom.

The present disclosure includes various means of engineering a portion of an editing nucleic acid of the present disclosure for integration into genomic DNA of a target or recipient subject, cell, or system. For example, an integrating fragment of an editing nucleic acid of the present disclosure can be present in a transposon that can be integrated into genomic DNA by a transposase. Transposons include a short nucleic acid sequence with terminal repeat sequences upstream and downstream of a larger segment of DNA. Transposases bind the terminal repeat sequences and catalyze the movement of the transposon to another portion of the genome. Transposases can include integrases from retrotransposons or of retroviral origin, as well as an enzyme that is a component of a functional nucleic acid-protein complex capable of transposition and which is mediating transposition. A transposition reaction includes a transposon and a transposase or an integrase enzyme.

A number of transposases have been described in the art that facilitate insertion of nucleic acids into the genome of vertebrates, including humans. Examples of such transposases include sleeping beauty (“SB”, e.g., derived from the genome of salmonid fish); piggyback (e.g., derived from lepidopteran cells and/or the Myotis lucifugus); mariner (e.g., derived from Drosophila); frog prince (e.g., derived from Rana pipiens); Tol1; Tol2 (e.g., derived from medaka fish); TcBuster (e.g., derived from the red flour beetle Tribolium castaneum), Helraiser, Himar1, Passport, Minos, Ac/Ds, PIF, Harbinger, Harbinger3-DR, HSmar1, and spinON.

The Piggy Bac (PB) transposase is a compact functional transposase protein that is described in, for example, Fraser et al., Insect Mol. Biol., 1996, 5, 141-51; Mitra et al., EMBO J., 2008, 27, 1097-1109; Ding et al., Cell, 2005, 122, 473-83; and U.S. Pat. Nos. 6,218,185; 6,551,825; 6,962,810; 7,105,343; and 7,932,088. Hyperactive piggyBac transposases are described in U.S. Pat. No. 10,131,885.

Additional information on DNA transposons can be found, for instance, in Muñoz-López & García Perez, Curr Genomics, 11 (2): 115-128, 2010.

Sleeping Beauty is described in Ivics et al., Cell 91, 501-510, 1997; Izsvak et al., J. Mol. Biol., 302 (1): 93-102, 2000; Geurts et al., Molecular Therapy, 8 (1): 108-117, 2003; Mates et al., Nature Genetics 41:753-761, 2009; and U.S. Pat. Nos. 6,489,458; 7,148,203; and 7,160,682; US Publication Nos. 2011/117072; 2004/077572; and 2006/252140. In certain embodiments, the Sleeping Beauty transposase enzyme is a Hyperactive Sleeping Beauty SB100x transposase enzyme. SB transposons are most efficiently transposed when present in circularized nucleic acid molecules (Yant et al., Nature Biotechnology, 20:999-1005, 2002).

Systematic mutagenesis studies have been undertaken to increase the activity of the SB transposase. For example, Yant et al., undertook the systematic exchange of the N-terminal 95 AA of the SB transposase for alanine (Mol. Cell Biol. 24:9239-9247, 2004). Ten of these substitutions caused hyperactivity between 200-400% as compared to SB10 as a reference. SB16, described in Baus et al., (Mol. Therapy 12:1148-1156, 2005) was reported to have a 16-fold activity increase as compared to SB10. Additional hyperactive SB variants are described in Zayed et al., (Molecular Therapy 9 (2): 292-304, 2004) and U.S. Pat. No. 9,840,696.

SB transposases transpose nucleic acid transposon payloads that are positioned between SB ITRs. Various SB ITRs are known in the art. In some embodiments, an SB ITR is a 230 bp sequence including imperfect direct repeats of 32 bp in length that serve as recognition signals for the transposase.

In various embodiments, an editing nucleic acid of the present disclosure includes a payload that includes SB100x transposon inverted repeats that flank an integrating payload that includes at least one coding sequence that encodes a β-globin expression product or a γ-globin expression product.

In various embodiments, a transposase can be provided to the same cell as the integrating payload by a further vector, where the transposase corresponds to the inverted repeats that flank the integrating payload. Accordingly, in various embodiments, a support vector or genome thereof can encode, express, and/or deliver to a target subject, cell, or system a transposase for transposition of an integrating payload present in an editing nucleic acid of the present disclosure.

In certain embodiments, an integrating payload is flanked by recombinase direct repeats, e.g., where the integrating payload is flanked by transposon inverted repeats and the transposon inverted repeats are flanked by recombinase direct repeats. In various embodiments, a recombinase can be provided to the same cell as the integrating payload by a further vector, where the recombinase corresponds to the direct repeats. Accordingly, in various embodiments, a support vector or genome thereof can encode, express, and/or deliver to a target subject, cell, or system a recombinase for recombination of recombinase sites present in an editing nucleic acid of the present disclosure.

Examples of recombinase systems include the Flp/Frt system, the Cre/loxP system, the Dre/rox system, the Vika/vox system, and the PhiC31 system. The Flp/Frt DNA recombinase system was isolated from Saccharomyces cerevisiae. The Flp/Frt system includes the recombinase Flp (flippase) that catalyzes DNA-recombination on its Frt recognition sites. Variants of the Flp protein include GenBank: ABD57356.1) and GenBank: ANW61888.1.

The Cre/loxP system is described in, for example, EP 02200009B1. Cre is a site-specific DNA recombinase isolated from bacteriophage P1. The recognition site of the Cre protein is a nucleotide sequence of 34 base pairs, the loxP site. Cre recombines the 34 bp loxP DNA sequence by binding to the 13 base pair inverted repeats and catalyzing strand cleavage and re-ligation within the spacer region. The staggered DNA cuts made by Cre in the spacer region are separated by 6 base pairs to give an overlap region that acts as a homology sensor to ensure that only recombination sites having the same overlap region recombine. Variants of the lox recognition site that can also be used include: lox2272; lox511; lox66; lox71; loxM2; and lox5171. The VCre/VloxP recombinase system was isolated from Vibrio plasmid p0908. The sCre/SloxP system is described in WO 2010/143606. The Dre/rox system is described in U.S. Pat. Nos. 7,422,889 and 7,915,037B2. It generally includes a Dre recombinase isolated from Enterobacteria phage D6 and the rox recognition site. The Vika/vox system is described in U.S. Pat. No. 10,253,332. Additionally, the PhiC31 recombinase recognizes the AttB/AttP binding sites.

Integration includes stable integration of an integrating payload into a target cell genome. By stable integration is meant that the nucleic acid remains present in the target cell genome for more than a transient period of time and can be passed on as part of the chromosomal genetic material to progeny of the target cell.

In various embodiments, an integrating payload is integrated into a genome by a process that utilizes homology arms to facilitate targeted insertion. In various embodiments, a double-strand break (DSB) in DNA (e.g., caused by an editing enzyme such as a CRISPR enzyme) can result in homology directed repair (HDR) at the site of the DSB based on a DNA template. Exogenous DNA provided and, if used as a template, cause a targeted change in the cleaved nucleic acid. Homology arms can be any length with sufficient homology to a genomic sequence at a cleavage site, e.g., 70%, 80%, 85%, 90%, 95%, or 100% homology with the nucleotide sequences flanking the cleavage site, e.g., within 50 bases or less of the cleavage site, e.g., within 30 bases, within 15 bases, within 10 bases, within 5 bases, or immediately flanking the cleavage site, to support HDR between the homology arms and the genomic sequence to which it bears homology. Homology arms are generally identical to genomic sequence, for example, to the genomic region in which the double-strand break (DSB) occurs. However, as indicated, absolute identity is not required.

In certain embodiments, integration of an integrating payload at specific genomic loci such as genomic safe harbors can include homology-directed integration using CRISPR enzyme-mediated cleavage of a target genome. CRISPR enzyme (e.g., Cas9) cleaves double-stranded DNA at a site specified by a guide RNA (gRNA). The double-strand break can be repaired by homology-directed repair (HDR) when a donor template is present. In various such methods, an integrating payload is a “repair template” in that it includes left and right homology arms (e.g., of 500-3,000 bp) for insertion into a cleaved target genome. CRISPR-mediated gene insertion can be several orders of magnitude more efficient compared with spontaneous recombination of DNA template, demonstrating that CRISPR-mediated gene insertion can be an effective tool for genome editing. Exemplary methods of homology-directed integration of a nucleic acid sequence into a specified genomic locus are known in the art, e.g., in Richardson et al., (Nat Biotechnol. 34 (3): 339-44, 2016).

Particular embodiments can utilize homology arms that have at least or about 25, 50, 100, or 200 nucleotides, or more than 200 nucleotides of sequence homology between an HDR template and a targeted genomic sequence (or any integral value between 10 and 200 nucleotides, or more). In particular embodiments, homology arms are 40-1000 nucleotides in length. In particular embodiments, homology arms are 500-2500 nucleotides, 700-2000 nucleotides, or 800-1800 nucleotides in length. In particular embodiments, homology arms include at least 800 nucleotides or at least 850 nucleotides. The length of homology arms can also be symmetric or asymmetric.

Particular embodiment can utilize first and/or second homology arms each including at least 25, 50, 100, 200, 400, 600, 800, 1,000, 1,200, 1,400, 1,600, 1,800, 2,000, 2,500, or 3,000 nucleotides or more, having sequence identity or homology with a corresponding fragment of a target genome. In some embodiments, first and/or second homology arms each include a number of nucleotides having sequence identity or homology with a corresponding fragment of a target genome that has a lower bound of 25, 50, 100, 200, 400, 600, 800, 1,000, 1,200, 1,400, 1,600, or 1,800 nucleotides and an upper bound of 1,000, 1,200, 1,400, 1,600, 1,800, 2,000, 2,500, or 3,000 nucleotides. In some embodiments, first and/or second homology arms each include a number of nucleotides having sequence identity or homology with a corresponding fragment of a target genome that is between 40 and 1,000 nucleotides, between 500 and 2,500 nucleotides, between 700 and 2,000 nucleotides, or between 800 and 1800 nucleotides, or that has a length of at least 800 nucleotides or at least 850 nucleotides. First and second homology arms can have same, similar, or different lengths.

In particular embodiments, integrating payloads (e.g., genes leading to expression of a therapeutic product within a cell) are precisely inserted within genomic safe harbors. Genomic safe harbor sites are intragenic or extragenic regions of the genome that are able to accommodate the predictable expression of newly integrated DNA without adverse effects on the host cell. A useful safe harbor must permit sufficient transgene expression to yield desired levels of the encoded protein. A genomic safe harbor site also must not alter cellular functions. Methods for identifying genomic safe harbor sites are described in Sadelain et al., Nature Reviews 12:51-58, 2012; and Papapetrou et al., Nat Biotechnol. 29 (1): 73-8, 2011. In particular embodiments, a genomic safe harbor site meets one or more (one, two, three, four, or five) of the following criteria: (i) distance of at least 50 kb from the 5′ end of any gene, (ii) distance of at least 300 kb from any cancer-related gene, (iii) within an open/accessible chromatin structure (measured by DNA cleavage with natural or engineered nucleases), (iv) location outside a gene transcription unit and (v) location outside ultraconserved regions (UCRs), microRNA or long non-coding RNA of the genome.

In particular embodiments, to meet the criteria of a genomic safe harbor, chromatin sites must be >150 kb away from a known oncogene, >30 kb away from a known transcription start site; and have no overlap with coding mRNA. In particular embodiments, to meet the criteria of a genomic safe harbor, chromatin sites must be >200 kb away from a known oncogene, >40 kb away from a known transcription start site; and have no overlap with coding mRNA. In particular embodiments, to meet the criteria of a genomic safe harbor, chromatin sites must be >300 kb away from a known oncogene, >50 kb away from a known transcription start site; and have no overlap with coding mRNA. In particular embodiments, a genomic safe harbor meets the preceding criteria (>150 kb, >200 kb or >300 kb away from a known transcription start site; and have no overlap with coding mRNA >40 kb, or >50 kb away from a known transcription start site with no overlap with coding mRNA) and additionally is 100% homologous between an animal of a relevant animal model and the human genome to permit rapid clinical translation of relevant findings.

In particular embodiments, a genomic safe harbor meets criteria described herein and also demonstrates a 1:1 ratio of forward:reverse orientations of lentiviral integration further demonstrating the locus does not impact surrounding genetic material.

Particular genomic safe harbors sites include CCR5, HPRT, AAVS1, Rosa and albumin. See also, e.g., U.S. Pat. Nos. 7,951,925 and 8,110,379; U.S. Publication Nos. 2008/0159996; 2010/00218264; 2012/0017290; 2011/0265198; 2013/0137104; 2013/0122591; 2013/0177983 and 2013/0177960 for additional information and options for appropriate genomic safe harbor integration sites.

Various technologies known in the art can be used to direct integration of an integrating payload at specific genomic loci such as genomic safe harbors. For example AAV-mediated gene targeting, as well as homologous recombination enhanced by the introduction of DNA double-strand breaks using site-specific endonucleases (zinc-finger nucleases, meganucleases, transcription activator-like effector (TALE) nucleases), and CRISPR/Cas systems are all tools that can mediate targeted insertion of foreign DNA at predetermined genomic loci such as genomic safe harbors.

One means of engineering vectors that integrate a payload into a host cell genome has been to produce integrating viral hybrid vectors. Integrating viral hybrid vectors combine genetic elements of a vector that efficiently transduces target cells with genetic elements of a vector that stably integrates its vector payload. Integrating payloads of interest, e.g., for use in combination with vectors, have included those of bacteriophage integrase PHIC31, retrotransposons, retrovirus (e.g., LTR-mediated or retrovirus integrate-mediated), zinc-finger nuclease, DNA-binding domain-retroviral integrase fusion proteins, AAV (e.g., AAV-ITR or AAV-Rep protein-mediated), and Sleeping Beauty (SB) transposase.

Expression products (e.g., editing system components and/or therapeutic payload expression products) encoded by editing nucleic acids of the present disclosure can be operably linked with one or more regulatory sequences optionally selected from a promoter, enhancer, insulator, termination signal, polyadenylation signal, splicing signal, and/or the like. Those of skill in the art will appreciate that methods and techniques for operably linking a regulatory sequence and a coding sequence are known. Various exemplary regulatory sequences, such as exemplary promoters, are provided herein by way of example.

A promoter can be a non-coding genomic DNA sequence, usually upstream (5′) to the relevant coding sequence, to which RNA polymerase binds before initiating transcription. This binding aligns the RNA polymerase so that transcription will initiate at a specific transcription initiation site. The nucleotide sequence of the promoter determines the nature of the enzyme and other related protein factors that attach to it and the rate of RNA synthesis. The RNA is processed to produce messenger RNA (mRNA) which serves as a template for translation of the RNA sequence into the amino acid sequence of the encoded polypeptide. The 5′ non-translated leader sequence is a region of the mRNA upstream of the coding region that may play a role in initiation and translation of the mRNA. The 3′ transcription termination/polyadenylation signal is a non-translated region downstream of the coding region that functions in the plant cell to cause termination of the RNA synthesis and the addition of polyadenylate nucleotides to the 3′ end.

Promoters can include general promoters, tissue-specific promoters, cell-specific promoters, and/or promoters specific for the cytoplasm. Promoters may include strong promoters, weak promoters, constitutive expression promoters, and/or inducible (conditional) promoters. Inducible promoters direct or control expression in response to certain conditions, signals, or cellular events. For example, the promoter may be an inducible promoter that requires a particular ligand, small molecule, transcription factor, hormone, or hormone protein in order to effect transcription from the promoter. Particular examples of promoters include the AFP (α-fetoprotein) promoter, amylase 1C promoter, aquaporin-5 (AP5) promoter, al-antitrypsin promoter, β-act promoter, β-globin promoter, β-Kin promoter, B29 promoter, CCKAR promoter, CD14 promoter, CD43 promoter, CD45 promoter, CD68 promoter, CEA promoter, c-erbB2 promoter, COX-2 promoter, CXCR4 promoter, desmin promoter, E2F-1 promoter, human elongation factor 1α promoter (EF1α), CMV (cytomegalovirus viral) promoter, minCMV promoter, SV40 (simian virus 40) immediately early promoter, EGR1 promoter, eIF4A1 promoter, elastase-1 promoter, endoglin promoter, FerH promoter, FerL promoter, fibronectin promoter, Flt-1 promoter, GAPDH promoter, GFAP promoter, GPIIb promoter, GRP78 promoter, GRP94 promoter, HE4 promoter, hGR1/1 promoter, hNIS promoter, Hsp68 promoter, the Hsp68 minimal promoter (proHSP68), HSP70 promoter, HSV-1 virus TK gene promoter, hTERT promoter, ICAM-2 promoter, kallikrein promoter, LP promoter, major late promoter (MLP), Mb promoter, Rho promoter, MT (metallothionein) promoter, MUC1 promoter, NphsI promoter, OG-2 promoter, PGK (Phospho Glycerate kinase) promoters, PGK-1 promoter, polymerase III (Pol III) promoter, PSA promoter, ROSA promoter, SP-B promoter, Survivn promoter, SYNI promoter, SYT8 gene promoter, TRP1 promoter, Tyr promoter, ubiquitin B promoter, WASP promoter, and the Rous Sarcoma Virus (RSV) long-terminal repeat (LTR) promoter

Promoters may be obtained as native promoters or composite promoters. Native promoters, or minimal promoters, refer to promoters that include a nucleotide sequence from the 5′ region of a given gene. A native promoter includes a core promoter and its natural 5′UTR. In particular embodiments, the 5′UTR includes an intron. Composite promoters refer to promoters that are derived by combining promoter elements of different origins or by combining a distal enhancer with a minimal promoter of the same or different origin.

In particular embodiments, promoters include wild type promoter sequences and sequences with optional changes (including insertions, point mutations or deletions) at certain positions relative to the wild-type promoter. In particular embodiments, promoters vary from naturally occurring promoters by having 1 change per 20 nucleotide stretch, 2 changes per 20 nucleotide stretch, 3 changes per 20 nucleotide stretch, 4 changes per 20 nucleotide stretch, or 5 changes per 20 nucleotide stretch. In particular embodiments, the natural sequence will be altered in 1, 2, 3, 4, 5, 6, 7, 8, 9, or 10 bases. The promoter may vary in length, including from 50 nucleotides of LTR sequence to 100, 200, 250 or 350 nucleotides of LTR sequence, with or without other viral sequence.

Some promoters are specific to a tissue or cell and some promoters are non-specific to a tissue or cell. Each gene in mammalian cells has its own promoter and some promoters can only be activated in certain cell types. A specific promoter aids in cell specific expression of a nucleotide sequence that is operably linked to the promoter sequence. A non-specific promoter, or ubiquitous promoter, aids in initiation of transcription of a gene or nucleotide sequence that is operably linked to the promoter sequence in a wide range of cells, tissues and cell cycles.

In some embodiments, the promoter is a non-specific promoter. In particular embodiments, a non-specific promoter includes CMV promoter, RSV promoter, SV40 promoter, mammalian elongation factor 1α (EF1α) promoter, β-act promoter, EGR1 promoter, eIF4A1 promoter, FerH promoter, FerL promoter, GAPDH promoter, GRP78 promoter, GRP94 promoter, HSP70 promoter, β-Kin promoter, PGK-1 promoter, ROSA promoter, and/or ubiquitin B promoter.

In various embodiments, a coding sequence is operably linked to a microRNA (or miRNA) control system. An miRNA control system can refer to a method or composition in which expression of a coding sequence is regulated by the presence of microRNA sites (e.g., nucleic acid sequences with which a microRNA can interact). In various embodiments, the present disclosure includes payload in which a nucleic acid sequence encoding an expression product is operably linked to an miRNA target site such that expression of the expression product is controlled by presence, level, activity, and/or contact with a corresponding miRNA. For the avoidance of doubt the present disclosure contemplates that a nucleic acid sequence operably linked with an miRNA site, e.g., as disclosed herein can be a nucleic acid sequence that encodes, e.g., any of one or more expression products provided herein.

### Nucleic Acid Delivery Vectors

The present disclosure includes various methods and compositions for delivery of nucleic acids encoding an editing system of the present disclosure. Vectors of the present disclosure include agents for delivery of a nucleic acid to a subject, cell, or system. In various embodiments, a nucleic acid encoding an editing system of the present disclosure is delivered by a vector such as a nanoparticle, lipid nanoparticle, liposome, plasmid, cosmid, virus, or phage.

In various embodiments, a nucleic acid encoding an editing system of the present disclosure is included in and/or associated with a nanoparticle. Nanoparticles (NPs) can range in size from 10 to 1000 nm. Various nanoparticles that can include nucleic acids are known in the art. Examples include noble metal NPs, nanorods (NRs), nanoclusters (NCs), semiconductor quantum dots (QDs), and carbon allotropes such as single-wall carbon nanotubes (SWCNTs) and graphene. Particular examples further include gold NPs, silver NPs, gold NRs, gold/silver hybrids, silver NCs, magnetic nanoparticles, platinum NPs, palladium NPs, graphene oxide, micelles, polyacrylamide NPs, viral NPs, ferritin NPs, upconversion NPs, chalcogenide NPs, alkaline earth metal NPs, and DNA NPs. The present disclosure further includes lipid nanoparticles (LNPs, e.g., solid lipid nanoparticles (SLNs) and nanostructured lipid carriers (NLCs)).

In various embodiments, a nucleic acid encoding an editing system of the present disclosure is encapsulated in an LNP. LNPs can include cationic lipids together with other components such as neutral phospholipids, phosphatidylcholines, sterols such as cholesterol, and/or PEGylated phospholipids. SLNs, produced using lipids that are solid at room temperature and at body temperature, are colloidal nanoparticles with a solid lipophilic core. The solid lipid core of an SLN can include triglycerides (e.g., tri-stearin), glyceride mixtures or partial glycerides (e.g., Imwitor), fatty acids (e.g., stearic acid or palmitic acid), steroids (e.g., cholesterol), and/or waxes (e.g., cetyl palmitate) that are solid at both room temperature and human body temperature. Lipid nano-emulsions (LNE) are colloidal nanoparticles with a core that is liquid at room temperature. NLCs include a mixture of solid and liquid lipids, such as glyceryl tricaprylate, ethyl oleate, isopropyl myristate, and/or glyceryl dioleate.

In various embodiments, a nucleic acid encoding an editing system of the present disclosure is encapsulated in a liposome. Liposomes can include one or more phospholipid bilayers. Phospholipids can be organized in a bilayer structure due to their amphipathic properties, forming vesicles. Phospholipids can include, for example, phosphatidyl choline (lecithin; PC), phosphatidyl ethanolamine (cephalin; PE), phosphatidyl serine (PS), phosphatidyl inositol (PI), and/or phosphatidyl glycerol (PG). Liposomes can further include additional agents such as cholesterol, lipid chains, and/or surfactants. In various embodiments, cholesterol does not form a bilayer by itself, but can incorporate into phospholipid membranes. In various embodiments, a liposome can include a hydrophilic carbohydrate or polymer, such as a lipid derivative of polyethylene glycol (PEG). Liposomes include conventional liposomes, pH sensitive liposomes, cationic liposomes, immune liposomes, and long circulating liposomes. Liposomes include multilamellar vesicles and unilamellar vesicles (e.g., large and small unilamellar vesicles).

In various embodiments, a nucleic acid encoding an editing system of the present disclosure is encapsidated in a viral particle of a virus. Various types of viruses can be used for delivery of nucleic acids. Viruses for delivery of a nucleic acid encoding an editing system of the present disclosure can be adenoviruses, adeno-associated viruses, alphaviruses, flaviviruses, herpes simplex viruses (HSV), measles viruses, rhabdoviruses, retroviruses, lentiviruses, Newcastle disease virus (NDV), poxviruses, and picornaviruses.

In certain embodiments, a nucleic acid encoding an editing system of the present disclosure is encapsidated in a viral particle of an adenovirus. Adenoviruses are large, icosahedral-shaped, non-enveloped viruses. Natural adenoviral capsids include three types of proteins: fiber, penton, and hexon. The hexon makes up the majority of the viral capsid, forming 20 triangular faces. A penton base is located at each of the 12 vertices of the capsid, and a fiber (also referred to as a knobbed fiber) protrudes from each penton base. Penton and fiber, and in particular the fiber knob, are of particular importance in receptor binding and internalization as they facilitate the attachment of the capsid to host cells. In various embodiments, the adenovirus is a helper-dependent adenovirus. In various embodiments, an adenoviral vector is a vector of a serotype selected from Ad3, Ad5, Ad7, Ad11, Ad14, Ad16, Ad21, Ad34, Ad35, Ad37, or Ad50.

In various embodiments, an Ad3, Ad5, Ad7, Ad11, Ad14, Ad16, Ad21, Ad34, Ad35, Ad37, or Ad50 vector is an adenoviral vector that includes a genome of the indicated serotype. In various embodiments, an Ad3, 5, 7, 11, 14, 16, 21, 34, 35, 37, or 50 genome is a single-stranded or double-stranded DNA sequence that includes ITRs of an Ad3, 5, 7, 11, 14, 16, 21, 34, 35, 37, or 50 vector (e.g., a 5′ ITR according to SEQ ID NO: 10, 28, 46, 64, 82, 100, 118, 136, 154, 172, or 190 and a 3′ ITR according to SEQ ID NO: 11, 29, 47, 65, 83, 101, 119, 137, 155, 173, or 191), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 5, 7, 11, 14, 16, 21, 34, 35, 37, or 50 genome is a single-stranded or double-stranded DNA sequence that includes a packaging sequence of an Ad3, 5, 7, 11, 14, 16, 21, 34, 35, 37, or 50 vector (e.g., a packaging sequence according to SEQ ID NO: 12, 30, 48, 66, 84, 102, 120, 138, 156, 174, or 192), or a packaging sequence having at least 75% sequence identity (e.g., at least 75%, 80%, 85%, 90%, 95%, 96%, 97%, 98%, 99%, or 100% identity) to the entirety of a portion thereof. In various embodiments, an Ad3, 5, 7, 11, 14, 16, 21, 34, 35, 37, or 50 genome is a single-stranded or double-stranded DNA sequence that includes a sequence with at least 75% sequence identity (e.g., at least 75%, 80%, 85%, 90%, 95%, 96%, 97%, 98%, 99%, or 100% identity) to all, a portion of, or a contiguous corresponding portion of, or a discontiguous corresponding portion of a reference Ad3, 5, 7, 11, 14, 16, 21, 34, 35, 37, or 50 genome (e.g., SEQ ID NO: 208, 209, 210, 211, 212, 213, 214, 215, 216, 217, or 218).

In various embodiments, an Ad3, 5, 7, 11, 14, 16, 21, 34, 35, 37, or 50 genome is any nucleotide sequence that includes at least ITRs of an Ad3, 5, 7, 11, 14, 16, 21, 34, 35, 37, or 50 vector (e.g., a 5′ITR according to SEQ ID NO: 10, 28, 46, 64, 82, 100, 118, 136, 154, 172, 190 and a 3′ ITR according to SEQ ID NO: 11, 29, 47, 65, 83, 101, 119, 137, 155, 173, or 191), or ITRs that individually and/or together have at least 75% sequence identity (e.g., at least 75%, 80%, 85%, 90%, 95%, 96%, 97%, 98%, 99%, or 100% identity) thereto. In various embodiments, an Ad3, 5, 7, 11, 14, 16, 21, 34, 35, 37, or 50 genome is an Ad3, 5, 7, 11, 14, 16, 21, 34, 35, 37, or 50 genome from which one or more nucleotides, coding sequences, and/or genes are completely or partially deleted as compared to a reference sequence. For example, in some embodiments, an Ad3, 5, 7, 11, 14, 16, 21, 34, 35, 37, or 50 genome can be a genome that does not include one or more of E1, E2, E3, and E4. In certain embodiments, an Ad3, 5, 7, 11, 14, 16, 21, 34, 35, 37, or 50 genome is a genome that does not include any coding sequences of an Ad3, 5, 7, 11, 14, 16, 21, 34, 35, 37, or 50 genome (e.g., a “gutless” vector that includes ITRs having at least 75% sequence identity to Ad3, 5, 7, 11, 14, 16, 21, 34, 35, 37, or 50 genome ITRs but includes none of the coding sequences present in a reference Ad3, 5, 7, 11, 14, 16, 21, 34, 35, 37, or 50 genome).

In various embodiments, an Ad3, 5, 7, 11, 14, 16, 21, 34, 35, 37, or 50 genome includes, does not include, or includes a deletion of, all or a portion of an E1 sequence according to SEQ ID NO: 13, 31, 49, 67, 85, 103, 121, 139, 157, 175, or 193, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, does not include, or includes a deletion of, all or a portion of an E2 sequence according to SEQ ID NO: 14, 32, 50, 68, 86, 104, 122, 140, 158, 176, or 194, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, does not include, or includes a deletion of, all or a portion of an E3 sequence according to SEQ ID NO: 15, 33, 51, 69, 87, 105, 123, 141, 159, 177, or 195, or a sequence having at least 75% sequence identity (e.g., at least 75%, 80%, 85%, 90%, 95%, 96%, 97%, 98%, 99%, or 100% identity) thereto.

In various embodiments, an Ad3, 5, 7, 11, 14, 16, 21, 34, 35, 37, or 50 genome includes, or does not include, a sequence that encodes a fiber, where the sequence has at least 75% sequence identity (e.g., at least 75%, 80%, 85%, 90%, 95%, 96%, 97%, 98%, 99%, or 100% identity) to SEQ ID NO: 16, 34, 52, 70, 88, 106, 124, 142, 160, 178, or 196.

In various embodiments, an Ad3, 5, 7, 11, 14, 16, 21, 34, 35, 37, or 50 genome includes, or does not include, a sequence that encodes a fiber tail, where the sequence has at least 75% sequence identity (e.g., at least 75%, 80%, 85%, 90%, 95%, 96%, 97%, 98%, 99%, or 100% identity) to a fiber tail of SEQ ID NO: 17, 35, 53, 71, 89, 107, 125, 143, 161, 179, or 197.

In various embodiments, an Ad3, 5, 7, 11, 14, 16, 21, 34, 35, 37, or 50 genome includes, or does not include, a sequence that encodes a fiber shaft, where the sequence has at least 75% sequence identity (e.g., at least 75%, 80%, 85%, 90%, 95%, 96%, 97%, 98%, 99%, or 100% identity) to SEQ ID NO: 18, 36, 54, 72, 90, 108, 126, 144, 162, 180, or 198.

In various embodiments, an Ad3, 5, 7, 11, 14, 16, 21, 34, 35, 37, or 50 genome includes, or does not include, a sequence that encodes a fiber knob, where the sequence has at least 75% sequence identity (e.g., at least 75%, 80%, 85%, 90%, 95%, 96%, 97%, 98%, 99%, or 100% identity) to SEQ ID NO: 19, 37, 55, 73, 91, 109, 127, 145, 163, 181, or 199.

In various embodiments, an Ad3, 5, 7, 11, 14, 16, 21, 34, 35, 37, or 50 genome includes, or does not include, a sequence that encodes a penton, where the sequence has at least 75% sequence identity (e.g., at least 75%, 80%, 85%, 90%, 95%, 96%, 97%, 98%, 99%, or 100% identity) to SEQ ID NO: 20, 38, 56, 74, 92, 110, 128, 146, 164, 182, or 200.

In various embodiments, an Ad3, 5, 7, 11, 14, 16, 21, 34, 35, 37, or 50 genome includes, or does not include, a sequence that encodes a hexon, where the sequence has at least 75% sequence identity (e.g., at least 75%, 80%, 85%, 90%, 95%, 96%, 97%, 98%, 99%, or 100% identity) to SEQ ID NO: 21, 39, 57, 75, 93, 111, 129, 147, 165, 183, or 201.

The present disclosure includes Ad3, 5, 7, 11, 14, 16, 21, 34, 35, 37, or 50 vectors that include a fiber having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber (e.g., a fiber according to SEQ ID NO: 22, 40, 58, 76, 94, 112, 130, 148, 166, 184, or 202).

The present disclosure includes Ad3, 5, 7, 11, 14, 16, 21, 34, 35, 37, or 50 vectors that include a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail of a fiber according to SEQ ID NO: 22, 40, 58, 76, 94, 112, 130, 148, 166, 184, or 202, e.g., where the fiber tail is the portion of the fiber including all amino acids N-terminal to the fiber shaft).

The present disclosure includes Ad3, 5, 7, 11, 14, 16, 21, 34, 35, 37, or 50 vectors that include a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail according to SEQ ID NO: 27, 45, 63, 81, 99, 117, 135, 153, 171, 189, or 207).

The present disclosure includes Ad3, 5, 7, 11, 14, 16, 21, 34, 35, 37, or 50 vectors that include a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft (e.g., a fiber shaft according to SEQ ID NO: 23, 41, 59, 77, 95, 113, 131, 149, 167, 185, or 203).

The present disclosure includes Ad3, 5, 7, 11, 14, 16, 21, 34, 35, 37, or 50 vectors that include a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob (e.g., a fiber knob according to SEQ ID NO: 24, 42, 60, 78, 96, 114, 132, 150, 168, 186, or 204).

The present disclosure includes Ad3, 5, 7, 11, 14, 16, 21, 34, 35, 37, or 50 vectors that include a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton (e.g., a penton according to SEQ ID NO: 25, 43, 61, 79, 97, 115, 133, 151, 169, 187, or 205).

The present disclosure includes Ad3, 5, 7, 11, 14, 16, 21, 34, 35, 37, or 50 vectors that include a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon (e.g., a hexon according to SEQ ID NO: 26, 44, 62, 80, 98, 116, 134, 152, 170, 188, or 206).

In various embodiments, an Ad3, 5, 7, 11, 14, 16, 21, 34, 35, 37, or 50 vector is any adenoviral vector that includes at least a fiber having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber (e.g., a fiber according to SEQ ID NO: 22, 40, 58, 76, 94, 112, 130, 148, 166, 184, or 202).

In various embodiments, an Ad3, 5, 7, 11, 14, 16, 21, 34, 35, 37, or 50 vector is any adenoviral vector that includes at least a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail of a fiber according to SEQ ID NO: 22, 40, 58, 76, 94, 112, 130, 148, 166, 184, or 202, e.g., where the fiber tail is the portion of the fiber including all amino acids N-terminal to the fiber shaft).

In various embodiments, an Ad3, 5, 7, 11, 14, 16, 21, 34, 35, 37, or 50 vector is any adenoviral vector that includes at least a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail (e.g., a fiber tail according to SEQ ID NO: 27, 45, 63, 81, 99, 117, 135, 153, 171, 189, or 207).

In various embodiments, an Ad3, 5, 7, 11, 14, 16, 21, 34, 35, 37, or 50 vector is any adenoviral vector that includes at least a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft (e.g., a fiber shaft according to SEQ ID NO: 23, 41, 59, 77, 95, 113, 131, 149, 167, 185, or 203).

In various embodiments, an Ad3, 5, 7, 11, 14, 16, 21, 34, 35, 37, or 50 vector is any adenoviral vector that includes at least a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob (e.g., a fiber knob according to SEQ ID NO: 24, 42, 60, 78, 96, 114, 132, 150, 168, 186, or 204).

In various embodiments, an Ad3, 5, 7, 11, 14, 16, 21, 34, 35, 37, or 50 vector is any adenoviral vector that includes at least a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton (e.g., a penton according to SEQ ID NO: 25, 43, 61, 79, 97, 115, 133, 151, 169, 187, or 205).

In various embodiments, an Ad3, 5, 7, 11, 14, 16, 21, 34, 35, 37, or 50 vector is any adenoviral vector that includes at least a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon (e.g., a hexon according to SEQ ID NO: 26, 44, 62, 80, 98, 116, 134, 152, 170, 188, or 206).

Thus, an Ad3, 5, 7, 11, 14, 16, 21, 34, 35, 37, or 50 vector can be a chimeric adenoviral vector that includes at least a fiber knob having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber knob and at least one protein or portion thereof (such as a fiber shaft, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a fiber shaft having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber shaft and at least one protein or portion thereof (such as a fiber knob, fiber tail, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a fiber tail having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 fiber tail and at least one protein or portion thereof (such as a fiber knob, fiber shaft, penton, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a penton having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 penton and at least one protein or portion thereof (such as a fiber knob, fiber shaft, fiber tail, or hexon) that has at least 75% sequence identity (e.g., at least 75%, 80%, 85%, 90%, 95%, 96%, 97%, 98%, 99%, or 100% identity) to a different adenoviral serotype.

An Ad3, 5, 7, 11, 14, 16, 21, 34, 35, 37, or 50 vector can be a chimeric adenoviral vector that includes at least a hexon having at least 75% sequence identity (e.g., at least 75%, 80%, 85%, 90%, 95%, 96%, 97%, 98%, 99%, or 100% identity) to an Ad3, 5, 7, 11, 14, 16, 21, 34, 35, 37, or 50 hexon and at least one protein or portion thereof (such as a fiber knob, fiber shaft, fiber tail, or penton) that has at least 75% sequence identity (e.g., at least 75%, 80%, 85%, 90%, 95%, 96%, 97%, 98%, 99%, or 100% identity) to a different adenoviral serotype.

Exemplary sequences of Ad3, 5, 7, 11, 14, 16, 21, 34, 35, 37, or 50 components (e.g., ITRs, packaging sequences, genes, and proteins) are provided in the following tables. Viral polypeptides include proteins that are components of viral vectors and portions or fragments thereof, including for example a fiber, fiber knob, fiber shaft, fiber tail, penton, or hexon.

In various embodiments, an Ad35 fiber knob of an Ad35 vector or chimeric Ad vector that includes an Ad35 fiber knob is a mutant Ad35 fiber knob. In particular embodiments, a mutant Ad35 fiber knob is an Ad35++ mutant fiber knob (alternatively referred to herein as an Ad35++ fiber knob). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593 (24): 3623-3648, 2019). In various embodiments, an Ad35++ mutant fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ mutant fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to GenBank Accession No. AP_000601 or an amino acid sequence corresponding thereto, e.g., where position 207 is Glu or Asp. In various embodiments, an Ad35 fiber has an amino acid sequence according to GenBank Accession No. AP_000601. Further description of Ad35++ fiber knob mutations is found in Wang 2008 J. Virol. 82 (21): 10567-10579, which is incorporated herein by reference in its entirety and with respect to fiber knobs. The present disclosure includes, for example, a recombinant Ad35 vector with a mutant Ad35 fiber knob or an Ad5/35 vector with a mutant Ad35 fiber knob.

In various embodiments, an adenoviral vector or genome of the present disclosure can be an adenoviral vector and/or genome disclosed in WO 2021/003432, which is herein incorporated by reference in its entirety, and particularly with respect to adenoviral vectors and genomes.

Various sequences corresponding to accession numbers disclosed herein, including e.g., accession sequences referred to herein as SEQ ID NOs: 208, 209, 210, 211, 212, 213, 214, 215, 216, 217, and/or 218 as indicated in Tables 3-24, are provided herein in the below listing of accession sequences. Those of skill in the art will appreciate that such sequences, including the sequences disclosed in the below listing of accession sequences, can be referenced in whole (e.g., by an accession number), or in part (e.g., by reference to a nucleotide position and/or a set or range of nucleotide positions of a sequence and/or accession number).

TABLE 3
Ad3 Genomic Sequences
Ad3 Genomic Sequences
Reference Ad3 Genome Sequence: GenBank
accession no. NC_011203 (SEQ ID NO: 208)

| | Exemplary Sequence | |
|---|---|---|
| Component | (position in reference) | SEQ ID NO: |
| Ad3 5′ (left) ITR | 1-136 | 10 |
| Ad3 3′ (right) ITR | 35208-35343 | 11 |
| Ad3 Packaging | 137-479 | 12 |
| Sequence | | |
| Ad3 E1 | 480-3918 | 13 |
| Ad3 E2 | 26643-3947 | 14 |
| Ad3 E3 | 27085-31186 | 15 |
| Ad3 fiber | 31368-32327 | 16 |
| Ad3 fiber tail | 31368-31493 | 17 |
| Ad3 fiber shaft | 31494-31763 | 18 |
| Ad3 fiber knob | 31764-32324 | 19 |
| Ad3 penton | 13905-15539 | 20 |
| Ad3 hexon | 18418-21252 | 21 |

TABLE 4
Ad3 Amino Acid Sequences
Ad3 Amino Acid Sequences

| | Exemplary Sequence | | |
|---|---|---|---|
| Component | (position in reference) | | SEQ ID NO: |
| Ad3 fiber | 1-319 | (GenBank accession no. YP_002213796) | 22 |
| Ad3 fiber shaft | 43-132 | (GenBank accession no. YP_002213796) | 23 |
| Ad3 fiber knob | 134-319 | (GenBank accession no. YP_002213796) | 24 |
| Ad3 penton | 1-544 | (GenBank accession no. YP_002213774) | 25 |
| Ad3 hexon | 1-944 | (GenBank accession no. YP_002213779) | 26 |
| Ad3 fiber tail | 1-42 | (GenBank accession no. YP_002213796) | 27 |

TABLE 5
Ad5 Genomic Sequences
Ad5 Genomic Sequences
Reference Ad5 Genome Sequence: GenBank
accession no. AC_000008 (SEQ ID NO: 209)

| | Exemplary Sequence | |
|---|---|---|
| Component | (position in reference) | SEQ ID NO: |
| Ad5 5′ (left) ITR | 1-103 | 28 |
| Ad5 3′ (right) ITR | 35836-35938 | 29 |
| Ad5 Packaging | 104-479 | 30 |
| Sequence | | |
| Ad5 E1 | 480-3509 | 31 |
| Ad5 E2 | 4091-24032 | 32 |
| Ad5 E3 | 27174-30839 | 33 |
| Ad5 fiber | 31042-32787 | 34 |
| Ad5 fiber tail | 31042-31170 | 35 |
| Ad5 fiber shaft | 31171-32241 | 36 |
| Ad5 fiber knob | 32242-32784 | 37 |
| Ad5 penton | 14156-15871 | 38 |
| Ad5 hexon | 18842-21700 | 39 |

TABLE 6
Ad5 Amino Acid Sequences
Ad5 Amino Acid Sequences

| | Exemplary Sequence | | SEQ |
|---|---|---|---|
| Component | (position in reference) | | ID NO: |
| Ad5 fiber | 1-581 | (GenBank accession no. AP_000226) | 40 |
| Ad5 fiber shaft | 44-400 | (GenBank accession no. AP_000226) | 41 |
| Ad5 fiber knob | 401-581 | (GenBank accession no. AP_000226) | 42 |
| Ad5 penton | 1-571 | (GenBank accession no. AP_000206) | 43 |
| Ad5 hexon | 1-952 | (GenBank accession no. AP_000211) | 44 |
| Ad5 fiber tail | 1-43 ( | GenBank accession no. AP_000226) | 45 |

TABLE 7
Ad7 Genomic Sequences
Ad7 Genomic Sequences
Reference Ad7 Genome Sequence: GenBank
accession no. AC_000018 (SEQ ID NO: 210)

| | Exemplary Sequence | SEQ |
|---|---|---|
| Component | (position in reference) | ID NO: |
| Ad7 5′ (left) ITR | 1-136 | 46 |
| Ad7 3′ (right) ITR | 35379-35514 | 47 |
| Ad7 Packaging | 137-479 | 48 |
| Sequence | | |
| Ad7 E1 | 480-3919 | 49 |
| Ad7 E2 | 26867-3947 | 50 |
| Ad7 E3 | 27308-31345 | 51 |
| Ad7 fiber | 31529-32506 | 52 |
| Ad7 fiber tail | 31529-31654 | 53 |
| Ad7 fiber shaft | 31655-31927 | 54 |
| Ad7 fiber knob | 31928-32503 | 55 |
| Ad7 penton | 14153-15787 | 56 |
| Ad7 hexon | 18666-21470 | 57 |

TABLE 8
Ad7 Amino Acid Sequences
Ad7 Amino Acid Sequences

| | Exemplary Sequence | | SEQ |
|---|---|---|---|
| Component | (position in reference) | | ID NO: |
| Ad7 fiber | 1-325 | (GenBank accession no. AP_000564) | 58 |
| Ad7 fiber shaft | 43-133 | (GenBank accession no. AP_000564) | 59 |
| Ad7 fiber knob | 134-325 | (GenBank accession no. AP_000564) | 60 |
| Ad7 penton | 1-544 | (GenBank accession no. AP_000543) | 61 |
| Ad7 hexon | 1-934 | (GenBank accession no. AP_000548) | 62 |
| Ad7 fiber tail | 1-42 | (GenBank accession no. AP_000564) | 63 |

TABLE 9
Ad11 Genomic Sequences
Ad11 Genomic Sequences
Reference Ad11 Genome Sequence: GenBank
accession no. NC_011202 (SEQ ID NO: 211)

| | Exemplary Sequence | |
|---|---|---|
| Component | (position in reference) | SEQ ID NO: |
| Ad11 5′ (left) ITR | 1-137 | 64 |
| Ad11 3′ (right) ITR | 34658-34794 | 65 |
| Ad11 Packaging | 138-479 | 66 |
| Sequence | | |
| Ad11 E1 | 480-3931 | 67 |
| Ad11 E2 | 25445-3963 | 68 |
| Ad11 E3 | 26866-30624 | 69 |
| Ad11 fiber | 30811-31788 | 70 |
| Ad11 fiber tail | 30811-30936 | 71 |
| Ad11 fiber shaft | 30937-31209 | 72 |
| Ad11 fiber knob | 31210-31785 | 73 |
| Ad11 penton | 13682-15367 | 74 |
| Ad11 hexon | 18254-21100 | 75 |

TABLE 10
Ad11 Amino Acid Sequences
Ad11 Amino Acid Sequences

| | Exemplary Sequence | | |
|---|---|---|---|
| Component | (position in reference) | | SEQ ID NO: |
| Ad11 fiber | 1-325 | (GenBank accession no. YP_002213828) | 76 |
| Ad11 fiber shaft | 43-133 | (GenBank accession no. YP_002213828) | 77 |
| Ad11 fiber knob | 134-325 | (GenBank accession no. YP_002213828) | 78 |
| Ad11 penton | 1-561 | (GenBank accession no. YP_002213807) | 79 |
| Ad11 hexon | 1-948 | (GenBank accession no. YP_002213812) | 80 |
| Ad11 fiber tail | 1-42 | (GenBank accession no. YP_002213828) | 81 |

TABLE 11
Ad14 Genomic Sequences
Ad14 Genomic Sequences
Reference Ad14 Genome Sequence:
GenBank accession no. AY803294 (SEQ ID NO: 212)

| | Exemplary Sequence | |
|---|---|---|
| Component | (position in reference) | SEQ ID NO: |
| Ad14 5′ (left) ITR | 1-137 | 82 |
| Ad14 3′ (right) ITR | 34628-34764 | 83 |
| Ad14 Packaging | 138-479 | 84 |
| Sequence | | |
| Ad14 E1 | 480-3947 | 85 |
| Ad14 E2 | 23389-3963 | 86 |
| Ad14 E3 | 26854-30601 | 87 |
| Ad14 fiber | 30788-31765 | 88 |
| Ad14 fiber tail | 30788-30913 | 89 |
| Ad14 fiber shaft | 30914-31186 | 90 |
| Ad14 fiber knob | 31187-31762 | 91 |
| Ad14 penton | 13698-15374 | 92 |
| Ad14 hexon | 18252-21089 | 93 |

TABLE 12
Ad14 Amino Acid Sequences
Ad14 Amino Acid Sequences

| | Exemplary Sequence | | |
|---|---|---|---|
| Component | (position in reference) | | SEQ ID NO: |
| Ad14 fiber | 1-325 | (GenBank accession no. AAW33140) | 94 |
| Ad14 fiber shaft | 43-133 | (GenBank accession no. AAW33140) | 95 |
| Ad14 fiber knob | 134-325 | (GenBank accession no. AAW33140) | 96 |
| Ad14 penton | 1-558 | (GenBank accession no. AAW33119) | 97 |
| Ad14 hexon | 1-945 | (GenBank accession no. AAW33124) | 98 |
| Ad14 fiber tail | 1-42 | (GenBank accession no. AAW33140) | 99 |

TABLE 13
Ad16 Genomic Sequences
Ad16 Genomic Sequences
Reference Ad16 Genome Sequence:
GenBank accession no. AY601636 (SEQ ID NO: 213)

| | Exemplary Sequence | |
|---|---|---|
| Component | (position in reference) | SEQ ID NO: |
| Ad16 5′ (left) ITR | 1-114 | 100 |
| Ad16 3′ (right) ITR | 35409-35522 | 101 |
| Ad16 Packaging | 115-479 | 102 |
| Sequence | | |
| Ad16 E1 | 480-3910 | 103 |
| Ad16 E2 | 23580-3954 | 104 |
| Ad16 E3 | 27107-31263 | 105 |
| Ad16 fiber | 31448-32509 | 106 |
| Ad16 fiber tail | 31448-31573 | 107 |
| Ad16 fiber shaft | 31574-31933 | 108 |
| Ad16 fiber knob | 31934-32506 | 109 |
| Ad16 penton | 13902-17534 | 110 |
| Ad16 hexon | 18450-21272 | 111 |

TABLE 14
Ad16 Amino Acid Sequences
Ad16 Amino Acid Sequences

| | Exemplary Sequence | | |
|---|---|---|---|
| Component | (position in reference) | | SEQ ID NO: |
| Ad16 fiber | 1-353 | (GenBank accession no. AAW33461) | 112 |
| Ad16 fiber shaft | 43-172 | (GenBank accession no. AAW33461) | 113 |
| Ad16 fiber knob | 173-353 | (GenBank accession no. AAW33461) | 114 |
| Ad16 penton | 1-555 | (GenBank accession no. AAW33439) | 115 |
| Ad16 hexon | 1-940 | (GenBank accession no. AAW33444) | 1160 |
| Ad16 fiber tail | 1-42 | (GenBank accession no. AAW33461) | 117 |

TABLE 15
Ad21 Genomic Sequences
Ad21 Genomic Sequences
Reference Ad21 Genome Sequence: GenBank
accession no. AY601633 (SEQ ID NO: 214)

| | Exemplary Sequence | |
|---|---|---|
| Component | (position in reference) | SEQ ID NO: |
| Ad21 5′ (left) ITR | 1-114 | 118 |
| Ad21 3′ (right) ITR | 35269-35382 | 119 |
| Ad21 Packaging | 115-479 | 120 |
| Sequence | | |
| Ad21 E1 | 480-3911 | 121 |
| Ad21 E2 | 23611-3924 | 122 |
| Ad21 E3 | 27441-31208 | 123 |
| Ad21 fiber | 31406-32377 | 124 |
| Ad21 fiber tail | 31406-31531 | 125 |
| Ad21 fiber shaft | 31532-31804 | 126 |
| Ad21 fiber knob | 31805-32374 | 127 |
| Ad21 penton | 13878-15563 | 128 |
| Ad21 hexon | 18454-21303 | 129 |

TABLE 16
Ad21 Amino Acid Sequences
Ad21 Amino Acid Sequences

| | Exemplary Sequence | | SEQ |
|---|---|---|---|
| Component | (position in reference) | | ID NO: |
| Ad21 fiber | 1-323 | (GenBank accession no. AAW33370) | 130 |
| Ad21 fiber shaft | 43-133 | (GenBank accession no. AAW33370) | 131 |
| Ad21 fiber knob | 134-323 | (GenBank accession no. AAW33370) | 132 |
| Ad21 penton | 1-561 | (GenBank accession no. AAW33349) | 133 |
| Ad21 hexon | 1-949 | (GenBank accession no. AAW33354) | 134 |
| Ad21 fiber tail | 1-42 | (GenBank accession no. AAW33370) | 135 |

TABLE 17
Ad34 Genomic Sequences
Ad34 Genomic Sequences
Reference Ad34 Genome Sequence: GenBank
accession no. AY737797 (SEQ ID NO: 215)

| | Exemplary Sequence | |
|---|---|---|
| Component | (position in reference) | SEQ ID NO: |
| Ad34 5′ (left) ITR | 1-137 | 136 |
| Ad34 3′ (right) ITR | 34639-34775 | 137 |
| Ad34 Packaging | 138-479 | 138 |
| Sequence | | |
| Ad34 E1 | 480-3929 | 139 |
| Ad34 E2 | 23399-3945 | 140 |
| Ad34 E3 | 27185-30625 | 141 |
| Ad34 fiber | 30812-31783 | 142 |
| Ad34 fiber tail | 30812-30937 | 143 |
| Ad34 fiber shaft | 30938-31210 | 144 |
| Ad34 fiber knob | 31211-31780 | 145 |
| Ad34 penton | 13681-15357 | 146 |
| Ad34 hexon | 18244-21099 | 147 |

TABLE 18
Ad34 Amino Acid Sequences
Ad34 Amino Acid Sequences

| | Exemplary Sequence | | SEQ |
|---|---|---|---|
| Component | (position in reference) | | ID NO: |
| Ad34 fiber | 1-323 | (GenBank accession no. AAW33501) | 148 |
| Ad34 fiber shaft | 43-133 | (GenBank accession no. AAW33501) | 149 |
| Ad34 fiber knob | 134-323 | (GenBank accession no. AAW33501) | 150 |
| Ad34 penton | 1-558 | (GenBank accession no. ABC49791) | 151 |
| Ad34 hexon | 1-951 | (GenBank accession no. AAW33485) | 152 |
| Ad34 fiber tail | 1-42 | (GenBank accession no. AAW33501) | 153 |

TABLE 19
Ad35 Genomic Sequences
Ad35 Genomic Sequences
Reference Ad35 Genome Sequence: GenBank
accession no. AY128640 (SEQ ID NO: 216)

| | Exemplary Sequence | |
|---|---|---|
| Component | (position in reference) | SEQ ID NO: |
| Ad35 5′ (left) ITR | 1-137 | 154 |
| Ad35 3′ (right) ITR | 34658-34794 | 155 |
| Ad35 Packaging | 138-479 | 156 |
| Sequence | | |
| Ad35 E1 | 480-3400 | 157 |
| Ad35 E2 | 3966-23415 | 158 |
| Ad35 E3 | 27198-30622 | 159 |
| Ad35 fiber | 30826-31797 | 160 |
| Ad35 fiber tail | 30826-30951 | 161 |
| Ad35 fiber shaft | 30952-31224 | 162 |
| Ad35 fiber knob | 31225-31797 | 163 |
| Ad35 penton | 13690-15375 | 164 |
| Ad35 hexon | 18255-21113 | 165 |

TABLE 20
Ad35 Amino Acid Sequences
Ad35 Amino Acid Sequences

| | Exemplary Sequence | | SEQ ID |
|---|---|---|---|
| Component | (position in reference) | | NO: |
| Ad35 fiber | 1-323 | (GenBank accession no. AP 000601) | 166 |
| Ad35 fiber shaft | 43-133 | (GenBank accession no. AP 000601) | 167 |
| Ad35 fiber knob | 134-323 | (GenBank accession no. AP 000601) | 168 |
| Ad35 penton | 1-561 | (GenBank accession no. AP 000580) | 169 |
| Ad35 hexon | 1-952 | (GenBank accession no. AP 000585) | 170 |
| Ad35 fiber tail | 1-42 | (GenBank accession no. AP 000601) | 171 |

TABLE 21
Ad37 Genomic Sequences
Ad37 Genomic Sequences
Reference Ad37 Genome Sequence: GenBank
accession no. DQ900900 (SEQ ID NO: 217)

| | Exemplary Sequence | |
|---|---|---|
| Component | (position in reference) | SEQ ID NO: |
| Ad37 5′ (left) ITR | 1-159 | 172 |
| Ad37 3′ (right) ITR | 35055-35213 | 173 |
| Ad37 Packaging | 160-479 | 174 |
| Sequence | | |
| Ad37 E1 | 480-3867 | 175 |
| Ad37 E2 | 22777-3902 | 176 |
| Ad37 E3 | 26198-30771 | 177 |
| Ad37 fiber | 31038-32135 | 178 |
| Ad37 fiber tail | 31038-31163 | 179 |
| Ad37 fiber shaft | 31164-31592 | 180 |
| Ad37 fiber knob | 31593-32132 | 181 |
| Ad37 penton | 13530-15089 | 182 |
| Ad37 hexon | 17775-20624 | 183 |

TABLE 22
Ad37 Amino Acid Sequences
Ad37 Amino Acid Sequences

| | Exemplary Sequence | | SEQ ID |
|---|---|---|---|
| Component | (position in reference) | | NO: |
| Ad37 fiber | 1-361 | (GenBank accession no. ABK59080) | 184 |
| Ad37 fiber shaft | 43-185 | (GenBank accession no. ABK59080) | 185 |
| Ad37 fiber knob | 186-361 | (GenBank accession no. ABK59080) | 186 |
| Ad37 penton | 1-519 | (GenBank accession no. ABK59086) | 187 |
| Ad37 hexon | 1-949 | (GenBank accession no. ABK59070) | 188 |
| Ad37 fiber tail | 1-42 | (GenBank accession no. ABK59080) | 189 |

TABLE 23
Ad50 Genomic Sequences
Ad50 Genomic Sequences
Reference Ad50 Genome Sequence: GenBank
accession no. AY737798 (SEQ ID NO: 218)

| | Exemplary Sequence | |
|---|---|---|
| Component | (position in reference) | SEQ ID NO: |
| Ad50 5′ (left) ITR | 1-114 | 190 |
| Ad50 3′ (right) ITR | 35272-35385 | 191 |
| Ad50 Packaging | 115-479 | 192 |
| Sequence | | |
| Ad50 E1 | 480-3910 | 193 |
| Ad50 E2 | 23590-3923 | 194 |
| Ad50 E3 | 27102-31222 | 195 |
| Ad50 fiber | 31409-32380 | 196 |
| Ad50 fiber tail | 31409-31534 | 197 |
| Ad50 fiber shaft | 31535-31807 | 198 |
| Ad50 fiber knob | 31808-32377 | 199 |
| Ad50 penton | 13888-15570 | 200 |
| Ad50 hexon | 18460-21282 | 201 |

TABLE 24
Ad50 Amino Acid Sequences
Ad50 Amino Acid Sequences

| | Exemplary Sequence | | SEQ |
|---|---|---|---|
| Component | (position in reference) | | ID NO: |
| Ad50 fiber | 1-323 | (GenBank accession no. AAW33547) | 202 |
| Ad50 fiber shaft | 43-133 | (GenBank accession no. AAW33547) | 203 |
| Ad50 fiber knob | 134-323 | (GenBank accession no. AAW33547) | 204 |
| Ad50 penton | 1-560 | (GenBank accession No. AAW33525) | 205 |
| Ad50 hexon | 1-940 | (GenBank accession no. AAW33530) | 206 |
| Ad50 fiber tail | 1-42 | (GenBank accession no. AAW33547) | 207 |

In various embodiments, an Ad3, 5, 7, 11, 14, 16, 21, 34, 35, 37, or 50 vector or genome includes modifications that reduce and/or eliminate replication of the virus in recipients. Broadly, there are three recognized “generations” of adenoviral vectors and genomes engineered to reduce and/or eliminate replication of the virus in recipients. Adenoviral vectors of the present disclosure can include vectors according to any of these three generations.

In various embodiments, an Ad3, 5, 7, 11, 14, 16, 21, 34, 35, 37, or 50 genome differs from a reference Ad sequence (e.g., one or more canonical, representative, exemplary, or wild-type sequence of an adenovirus of a serotype of interest) at least in that the regulatory E1 gene (E1a and E1b) is removed from the Ad genome (“first generation” vector modifications). E1a and E1b are the first transcriptional regulatory factors produced during the adenoviral replication cycle. E1 deletion reduces or eliminates expression of certain viral genes controlled by E1, and E1-deleted helper viruses are replication-defective. Thus, first generation Ad vectors are deficient for replication in a recipient. In some embodiments, first-generation adenoviral vectors are engineered to remove E1 and E3 genes. Retained portions of the reference genome can be identical in sequence to a reference genome or can have less than 100% identity with a reference genome, e.g., at least 99%, 98%, 97%, 96%, 95%, 90%, 85%, 80%, or 75% identity. Without these E1 (or E1 and E3) genes, adenoviral vectors cannot replicate on their own but can be produced in mammalian cell lines that express E1 (e.g., of the same serotype) or another protein sufficient to restore expression of the certain viral genes. For illustration, where an E1-deficient Ad5 vector encodes an Ad5 E4orf6, the helper vector can be propagated in a cell line that expresses Ad5 E1. In one exemplary cell type for adenoviral vector production, HEK293 cells express Ad5 E1b55k, which is known to form a complex with Ad5 E4 protein ORF6.

In various embodiments, an Ad3, 5, 7, 11, 14, 16, 21, 34, 35, 37, or 50 genome differs from a reference Ad sequence at least in that the E1 gene (E1a and E1b) and one or more of non-structural genes E2, E3 and/or E4 are deleted (“second generation” modifications). Second generation Ads have greater payload capacity than first generation Ads and are more deficient for replication than first generation viruses. In some embodiments, second-generation adenoviral vectors, in addition to E1/E3 removal, are engineered to remove non-structural genes E2 and E4, resulting in increased capacity and reduced immunogenicity. Retained portions of the reference genome can be identical in sequence to a reference genome or can have less than 100% identity with a reference genome, e.g., at least 99%, 98%, 97%, 96%, 95%, 90%, 85%, 80%, or 75% identity.

In various embodiments, an Ad3, 5, 7, 11, 14, 16, 21, 34, 35, 37, or 50 genome differs from a reference Ad sequence at least in that they are engineered to remove all viral coding sequences from the Ad genome, and retain only the ITRs of the genome and the packaging sequence of the genome or a functional fragment thereof (“third generation” modifications). Third generation adenoviral vectors can also be referred to as gutless, high capacity adenoviral vectors, or helper-dependent adenoviral vectors (HdAds). Retained portions of the reference genome can be identical in sequence to a reference genome or can have less than 100% identity with a reference genome, e.g., at least 99%, 98%, 97%, 96%, 95%, 90%, 85%, 80%, or 75% identity.

Because third generation Ad genomes do not encode the proteins necessary for viral production, they are helper-dependent: a helper-dependent genome can only be packaged into a vector if they are present in a cell that includes a nucleic acid sequence that provides viral proteins in trans. These helper-dependent vectors are also characterized by still greater capacity than first and second generation vectors and decreased immunogenicity. Because HDAd vectors do not express viral genes when used as a vector, the risk of cytotoxicity or interferon response in recipients is reduced.

Helper-dependent adenoviral vectors (HDAd) engineered to lack all viral coding sequences can efficiently transduce a wide variety of cell types, and can mediate long-term transgene expression with negligible chronic toxicity. By deleting the viral coding sequences and leaving only the cis-acting elements necessary for genome replication (ITRs) and packaging (v), cellular immune response against the Ad vector is reduced. HDAd vectors have a large cloning capacity of up to allowing for the delivery of large payloads. These payloads can include large therapeutic genes or even multiple transgenes and large regulatory components to enhance, prolong, and regulate transgene expression. It has also been observed that the certain HDAd vector genomes can be most efficiently packaged when the genome has at least a minimum a total length, e.g., a minimum to total length of at least 20 kb (e.g., 20, 21, 22, 23, 24, 25, 26, 27, 28, 29, 30, 31, 32, 33, 34, or 35 kb) which length can include, e.g., a therapeutic payload and/or a “stuffer” sequence. Where a payload does not utilize a number of nucleotides that causes the adenoviral genome to have at least a target length, a stuffer sequence can be used to achieve or surpass the target length. The present disclosure includes that a minimum length for efficient packaging is not required for beneficial use of vectors provided herein, such that meeting any target length may be advantageous but not required for use of compositions and methods provided herein. Like other adenoviral vectors, typical HDAd genomes generally remain episomal and do not integrate with a host genome.

Because HDAd vectors do not encode the viral proteins required to produce viral particles, viral proteins must be provided in trans, e.g., expressed in and/or by cells in which the HDAd genome is present. In some HDAd vector systems, one viral genome (a helper genome) encodes all of the proteins (e.g., all of the structural viral proteins) required for replication but has a conditional defect in the packaging sequence, making it less likely to be packaged into a vector under certain vector production conditions (e.g., in the presence of an agent that reduces function of the conditionally defective packaging sequence). Thus, the HDAd donor viral genome includes (e.g., only includes) Ad ITRs, a payload (e.g., a therapeutic payload), and a functional packaging sequence (e.g., a wild-type packaging sequence or a functional fragment thereof), which allows the HDAd donor viral genome to be selectively packaged into HDAd viral vectors produced from structural components expressed from the helper vector genome. In other words, Ad3, 5, 7, 11, 14, 16, 21, 34, 35, 37, or 50 helper vectors can be used for production of Ad3, 5, 7, 11, 14, 16, 21, 34, 35, 37, or 50 donor vectors. Production of HD Ad3, 5, 7, 11, 14, 16, 21, 34, 35, 37, or 50 vectors can include co-transfection of a plasmid containing the HDAd vector genome and a packaging-defective helper virus that provides structural and non-structural viral proteins. The helper virus genome can rescue propagation of the Ad3, 5, 7, 11, 14, 16, 21, 34, 35, 37, or 50 donor vector and Ad3, 5, 7, 11, 14, 16, 21, 34, 35, 37, or 50 donor vector can be produced, e.g., at a large scale, and isolated. Various protocols are known in the art, e.g., at Palmer et al., 2009 Gene Therapy Protocols. Methods in Molecular Biology, Volume 433. Humana Press; Totowa, NJ: 2009. pp. 33-53. In some embodiments, a helper genome is E1-deficient.

In some HDAd vector systems, a helper genome utilizes a recombinase system (e.g., a Cre/loxP system) for conditional packaging. In certain such HDAd vector systems, a helper genome can include a packaging sequence or functional fragment thereof (e.g., a fragment of the packaging sequence that is sufficient for packaging, required for packaging, or required for efficient packaging of the Ad genome into the capsid) flanked by recombinase (e.g., loxP) sites so that contact with a corresponding recombinase (e.g., Cre recombinase) excises the packaging sequence or functional fragment thereof from the helper genome by recombinase-mediated (e.g., Cre-mediated) site-specific recombination between the recombinase sites (e.g., loxP sites). The present disclosure includes, among other things, Ad3, 5, 7, 11, 14, 16, 21, 34, 35, 37, or 50 helper vectors and genomes that include two recombination sites that flank a packaging sequence or functional fragment thereof, where the two recombination sites are sites corresponding to (i.e., for, or acted upon by) the same recombinase.

In various embodiments, a helper genome can include deletion of E1, e.g., where the helper genome includes all of the viral genes except for E1, as E1 expression products can be supplied by complementary expression from the genome of a producer cell line. In some embodiments, to prevent generation of replication competent Ad (RCA) as a consequence of homologous recombination between the helper and HDAd donor genomes present in producer cells, a “stuffer” sequence can be inserted into the E3 region to render any recombinants too large to be packaged and/or efficiently packaged.

For production of HDAd vectors, an HDAd donor genome can be delivered to cells that express a recombinase for excision of the conditional packaging sequence of a helper vector (e.g., 293 cells (HEK293) that expresses Cre recombinase), optionally where the HDAd donor genome is delivered to the cells in a non-viral vector form, such as a bacterial plasmid form (e.g., where the HDAd donor genome is present in a bacterial plasmid (pHDAd) and/or is liberated by restriction enzyme digestion). The same cells can be transduced with the helper genome including a packaging sequence or functional fragment thereof flanked by recombinase sites (e.g., loxP sites). Thus, producer cells can be transfected with the HDAd donor genome and transduced with a helper genome bearing a packaging sequence or a functional fragment thereof flanked by recombinase sites (e.g., loxP sites), where the cells express a recombinase (e.g., Cre) corresponding to the recombinase sites such that excision of the packaging sequence or functional fragment thereof renders the helper virus genome deficient for packaging (e.g., unpackageable), but still able to provide all of the necessary trans-acting factors for production of HDAd donor vector including the HDAd donor genome.

Similar HDAd production systems have been developed using FLP (e.g., FLPe)/frt site-specific recombination, where FLP-mediated recombination between frt sites flanking the packaging sequence or functional fragment thereof of the helper genome reduces or eliminates packaging of helper genomes in producer cells that express FLP.

HDAd vectors including the donor vector genome including the payload can be isolated from the producer cells. HDAd donor vectors can be further purified from helper vectors by physical means. In general, some contamination of helper vectors and/or helper genomes in HDAd viral vectors and HDAd viral vector formulations can occur and can be tolerated.

HDAd3, 5, 7, 11, 14, 16, 21, 34, 35, 37, and 50 donor vectors, donor genomes, helper vectors, and helper genomes are also exemplary of compositions provided herein and can be used in various methods of the present disclosure. An HDAd3, 5, 7, 11, 14, 16, 21, 34, 35, 37, or 50 vector or genome is a helper-dependent Ad3, 5, 7, 11, 14, 16, 21, 34, 35, 37, or 50 vector or genome. An Ad3, 5, 7, 11, 14, 16, 21, 34, 35, 37, or 50 helper vector is a vector that includes a helper genome that includes a conditionally expressed (e.g., frt-site or loxP-site flanked) packaging sequence or fragment thereof and encodes all of the necessary trans-acting factors for production of Ad3, 5, 7, 11, 14, 16, 21, 34, 35, 37, or 50 virions into which the donor genome can be packaged.

The present disclosure further includes an HDAd3, 5, 7, 11, 14, 16, 21, 34, 35, 37, or 50 donor vector production system including a cell including an HDAd3, 5, 7, 11, 14, 16, 21, 34, 35, 37, or 50 donor genome and an Ad3, 5, 7, 11, 14, 16, 21, 34, 35, 37, or 50 helper genome. In certain such cells, viral proteins encoded and expressed by the helper genome can be utilized in production of HDAd3, 5, 7, 11, 14, 16, 21, 34, 35, 37, or 50 donor vectors in which the HDAd3, 5, 7, 11, 14, 16, 21, 34, 35, 37, or 50 donor genome is packaged. Accordingly, the present disclosure includes methods of production of HDAd3, 5, 7, 11, 14, 16, 21, 34, 35, 37, or 50 donor vectors by culturing cells that include an HDAd3, 5, 7, 11, 14, 16, 21, 34, 35, 37, or 50 donor genome and an Ad3, 5, 7, 11, 14, 16, 21, 34, 35, 37, or 50 helper genome. In some embodiments, the cells encode and express a recombinase that corresponds to recombinase direct repeats that flank a packaging sequence of the Ad3, 5, 7, 11, 14, 16, 21, 34, 35, 37, or 50 helper vector. In some embodiments, the flanked packaging sequence of the Ad3, 5, 7, 11, 14, 16, 21, 34, 35, 37, or 50 helper genome has been excised.

In some embodiments, the Ad3, 5, 7, 11, 14, 16, 21, 34, 35, 37, or 50 helper genome encodes all Ad3, 5, 7, 11, 14, 16, 21, 34, 35, 37, or 50 coding sequences. In some embodiments, the Ad3, 5, 7, 11, 14, 16, 21, 34, 35, 37, or 50 helper genome encodes and/or expresses all Ad3, 5, 7, 11, 14, 16, 21, 34, 35, 37, or 50 coding sequences except for one or more coding sequences of E1 and/or an E3 coding sequence and/or an E4 coding sequence. In various embodiments, a helper genome that does not encode and/or express an Ad3, 5, 7, 11, 14, 16, 21, 34, 35, 37, or 50 E1 gene does not encode and/or express an Ad3, 5, 7, 11, 14, 16, 21, 34, 35, 37, or 50 E4 gene. In various embodiments, as will be appreciate by those of skill in the art, cells of compositions and methods for production of HDAd donor vectors can be cells that express an E1 expression product.

The present disclosure includes, among other things, HDAd3, 5, 7, 11, 14, 16, 21, 34, 35, 37, or 50 donor vectors and genomes that include Ad3, 5, 7, 11, 14, 16, 21, 34, 35, 37, or 50 ITRs (a 5′ Ad3, 5, 7, 11, 14, 16, 21, 34, 35, 37, or 50 ITR and a 3′ ITR of the same serotype), e.g., where two Ad3, 5, 7, 11, 14, 16, 21, 34, 35, 37, or 50 ITRs flank a packaging sequence and a payload. The present disclosure includes, among other things, HDAd3, 5, 7, 11, 14, 16, 21, 34, 35, 37, or 50 donor vectors and genomes in which E1 or a fragment thereof is deleted. The present disclosure includes, among other things, HDAd3, 5, 7, 11, 14, 16, 21, 34, 35, 37, or 50 vectors and genomes in which E3 or a fragment thereof is deleted.

In various embodiments, excision of a packaging sequence or functional fragment thereof from an Ad3, 5, 7, 11, 14, 16, 21, 34, 35, 37, or 50 helper genome reduces propagation of the vector by, e.g., at least 20%, 30%, 40%, 50%, 60%, 70%, 80%, 90%, 91%, 92%, 93%, 94%, 95%, 96%, 97%, 98%, 99%, 99.5%, 99.9%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, 99.5%, 99.9% or 100%), optionally where percent propagation is measured as the number of viral particles produced by propagation of excised vector (vector from which the recombinase site-flanked sequence has been excised) as compared to complete vector (vector from which the recombinase site-flanked sequence has not been excised) or as compared to wild-type Ad3, 5, 7, 11, 14, 16, 21, 34, 35, 37, or 50 vector under comparable conditions.

An additional optional engineering consideration can be engineering of a helper genome having a size that permits separation of helper vector from HDAd3, 5, 7, 11, 14, 16, 21, 34, 35, 37, or 50 donor vector by centrifugation, e.g., by CsCl ultracentrifugation. One means of achieving this result is to increase the size of the helper genome as compared to a typical Ad3, 5, 7, 11, 14, 16, 21, 34, 35, 37, or 50 genome. In particular, adenoviral genomes can be increased by engineering to at least 104% of wild-type length. Certain helper vectors of the present disclosure can accommodate a payload and/or stuffer sequence.

The present disclosure includes that in various embodiments a vector or genome of the present disclosure can include a selection of components each selected from, or having at least 75% sequence identity (e.g., at least 75%, 80%, 85%, 90%, 95%, 96%, 97%, 98%, 99%, or 100% identity) to, a corresponding sequence of a single particular serotype. To provide an illustrative example, all components can correspond to (e.g., have at least 75% sequence identity to sequences of) Ad34, excepting sequences otherwise indicated (e.g., a payload, e.g., a heterologous payload).

In various embodiments, a vector of the present disclosure is an HDAd5/35 vector that includes Ad5 capsid proteins except that the fibers are chimeric in that they include an Ad5 fiber tail, an Ad35 fiber shaft, and an Ad35 fiber knob, optionally wherein the Ad35 fiber knob is mutated for increased affinity to CD46 (e.g., Ad5/35++). In particular embodiments, an Ad5/35++ vector is a chimeric Ad5/35 vector with a mutant Ad35++ fiber knob (see, e.g., Wang et al., 2008 J. Virol. 82 (21): 10567-79, which is incorporated herein by reference in its entirety and particularly with respect to fiber knob mutations). In various embodiments, an Ad35++ mutant fiber knob is an Ad35 fiber knob mutated to increase the affinity to CD46, e.g., by 25-fold, e.g., such that the Ad35++ mutant fiber knob increases cell transduction efficiency, e.g., at lower multiplicity of infection (MOI) (Li and Lieber, FEBS Letters, 593 (24): 3623-3648, 2019). In various embodiments, an adenoviral vector or genome of the present disclosure can be an adenoviral vector and/or genome disclosed in WO 2021/003432, which is herein incorporated by reference in its entirety, and particularly with respect to adenoviral vectors and genomes.

### Gene Therapy

Methods and compositions provided herein are disclosed at least in part for use in in vivo, in vitro, and or ex vivo gene therapy. As used herein, the term in vivo has its art-recognized meaning, such that in vivo modification and in vivo gene therapy include modification of a cell and/or a component thereof (e.g., the genome of a cell or an mRNA molecule expressed therefrom) that is present in a living multicellular (e.g., mammalian, e.g., human) organism. However, for the avoidance of doubt, the present disclosure expressly includes the use of compositions and methods provided herein for ex-vivo engineering of cells and/or tissues, as well as in vitro uses including the engineering of cells and/or tissues for research purposes. As used herein, the term in vitro has its art-recognized meaning, such that in vitro modification and in vitro gene therapy include modification of a cell and/or a component thereof (e.g., the genome of a cell or an mRNA molecule expressed therefrom) that is present in an artificial environment (e.g., in a test tube, reaction vessel, incubator, or cell culture, etc.), rather than within a living multicellular (e.g., mammalian, e.g., human) organism. As used herein, the term ex vivo has its art-recognized meaning, such that ex vivo modification and ex vivo gene therapy include modification of a cell and/or a component thereof (e.g., the genome of a cell or an mRNA molecule expressed therefrom) that is present in an artificial environment (e.g., in a test tube, reaction vessel, incubator, or cell culture, etc.), rather than within a multicellular (e.g., mammalian, e.g., human) organism, after separation of the cell or component thereof from a multicellular (e.g., mammalian, e.g., human) organism. Gene therapy includes use of a vector, nucleic acid, and/or editing system of the present disclosure in a method of engineering a nucleic acid of a host cell (such as a target cell). Because such compositions and methods are of general utility, e.g., in gene therapy, they are useful both as tools in gene therapy in general and in various particular conditions, including those provided herein.

In vivo gene therapy is an attractive approach because it may not require any genotoxic conditioning (or could require less genotoxic conditioning) or ex vivo cell processing and thus could be adopted at many institutions worldwide, including those in developing countries, as the therapy could be administered through an injection, similar to platforms already used worldwide for delivery of vaccines. In various embodiments, methods of in vivo gene therapy with vectors of the present disclosure can include one or more steps of (i) target cell mobilization, (ii) immunosuppression, (iii) administration of a vector and/or editing system provided herein, and/or (iv) selection of modified cells.

Editing systems, nucleic acids, and vectors disclosed herein can be used for treating subjects (humans, veterinary animals (dogs, cats, reptiles, birds, etc.), livestock (horses, cattle, goats, pigs, chickens, etc.), and research animals (monkeys, rats, mice, fish, etc.). Treating subjects includes delivering therapeutically effective amounts of one or more editing systems, nucleic acids, or vectors of the present disclosure. Therapeutically effective amounts include those that provide therapeutic benefit in the treatment of a disease, disorder, or condition.

Vectors described herein can be administered in coordination with mobilization factors. In certain embodiments, vector formulations described herein can be administered in concert with HSPC and/or HSC mobilization. In particular embodiments, administration of vectors occurs concurrently with administration of one or more mobilization factors. In particular embodiments, administration of vector follows administration of one or more mobilization factors. In particular embodiments, administration of vector follows administration of a first one or more mobilization factors and occurs concurrently with administration of a second one or more mobilization factors. Agents for HSPC and/or HSC mobilization include, for example, granulocyte-colony stimulating factor (G-CSF), granulocyte macrophage colony stimulating factor (GM-CSF), AMD3100, SCF, S-CSF, a CXCR4 antagonist, a CXCR2 agonist, and Gro-Beta (GRO-β). In various embodiments, a CXCR4 antagonist is AMD3100 and/or a CXCR2 agonist is GRO-β.

In certain embodiments, vector formulations described herein are not administered in concert with HSPC and/or HSC mobilization, e.g., when it is desired to deliver an editing nucleic acid to cells other than HSPCs and/or HSCs.

G-CSF is a cytokine whose functions in HSPC and/or HSC mobilization can include the promotion of granulocyte expansion and both protease-dependent and independent attenuation of adhesion molecules and disruption of the SDF-1/CXCR4 axis. In particular embodiments, any commercially available form of G-CSF known to one of ordinary skill in the art can be used in the methods and formulations as disclosed herein, for example, Filgrastim (Neupogen®, Amgen Inc., Thousand Oaks, CA) and PEGylated Filgrastim (Pegfilgrastim, NEULASTA®, Amgen Inc., Thousand Oaks, CA).

GM-CSF is a monomeric glycoprotein also known as colony-stimulating factor 2 (CSF2) that functions as a cytokine and is naturally secreted by macrophages, T cells, mast cells, natural killer cells, endothelial cells, and fibroblasts. In particular embodiments, any commercially available form of GM-CSF known to one of ordinary skill in the art can be used in the methods and formulations as disclosed herein, for example, Sargramostim (Leukine, Bayer Healthcare Pharmaceuticals, Seattle, WA) and molgramostim (Schering-Plough, Kenilworth, NJ).

AMD3100 (MOZOBIL™, PLERIXAFOR™; Sanofi-Aventis, Paris, France), a synthetic organic molecule of the bicyclam class, is a chemokine receptor antagonist and reversibly inhibits SDF-1 binding to CXCR4, promoting HSPC and/or HSC mobilization. AMD3100 is approved to be used in combination with G-CSF for HSPC and/or HSC mobilization in patients with myeloma and lymphoma.

SCF, also known as KIT ligand, KL, or steel factor, is a cytokine that binds to the c-kit receptor (CD117). SCF can exist both as a transmembrane protein and a soluble protein. This cytokine plays an important role in hematopoiesis, spermatogenesis, and melanogenesis. In particular embodiments, any commercially available form of SCF known to one of ordinary skill in the art can be used in the methods and formulations as disclosed herein, for example, recombinant human SCF (Ancestim, STEMGEN®, Amgen Inc., Thousand Oaks, CA).

Chemotherapy used in intensive myelosuppressive treatments also mobilizes HSPCs to the peripheral blood as a result of compensatory neutrophil production following chemotherapy-induced aplasia. In particular embodiments, chemotherapeutic agents that can be used for mobilization of HSPCs and/or HSCs include cyclophosphamide, etoposide, ifosfamide, cisplatin, and cytarabine.

Additional agents that can be used for cell mobilization include: CXCL12/CXCR4 modulators (e.g., CXCR4 antagonists: POL6326 (Polyphor, Allschwil, Switzerland), a synthetic cyclic peptide which reversibly inhibits CXCR4; BKT-140 (4F-benzoyl-TN14003; Biokine Therapeutics, Rehovit, Israe); TG-0054 (Taigen Biotechnology, Taipei, Taiwan); CXCL12 neutralizer NOX-A12 (NOXXON Pharma, Berlin, Germany) which binds to SDF-1, inhibiting its binding to CXCR4); Sphingosine-1-phosphate (SIP) agonists (e.g., SEW2871, Juarez et al., Blood 119:707-716, 2012); vascular cell adhesion molecule-1 (VCAM) or very late antigen 4 (VLA-4) inhibitors (e.g., Natalizumab, a recombinant humanized monoclonal antibody against α4 subunit of VLA-4 (Zohren et al., Blood 111:3893-3895, 2008); BIO5192, a small molecule inhibitor of VLA-4 (Ramirez et al., Blood 114:1340-1343, 2009)); parathyroid hormone (Brunner et al., Exp Hematol. 36:1157-1166, 2008); proteasome inhibitors (e.g., Bortezomib, Ghobadi et al., ASH Annual Meeting Abstracts. p. 583, 2012); Groβ, a member of CXC chemokine family which stimulates chemotaxis and activation of neutrophils by binding to the CXCR2 receptor (e.g., SB-251353, King et al., Blood 97:1534-1542, 2001); stabilization of hypoxia inducible factor (HIF) (e.g., FG-4497, Forristal et al., ASH Annual Meeting Abstracts. p. 216, 2012); Firategrast, an α4β1 and α4β7 integrin inhibitor (α4β1/7) (Kim et al., Blood 128:2457-2461, 2016); Vedolizumab, a humanized monoclonal antibody against the α4β7 integrin (Rosario et al., Clin Drug Investig 36:913-923, 2016); and BOP (N-(benzenesulfonyl)-L-prolyl-L-O-(1-pyrrolidinylcarbonyl) tyrosine) which targets integrins α9β1/α4β1 (Cao et al., Nat Commun 7:11007, 2016). Additional agents that can be used for HSPC and/or HSC mobilization are described in, for example, Richter R et al., Transfus Med Hemother 44:151-164, 2017, Bendall & Bradstock, Cytokine & Growth Factor Reviews 25:355-367, 2014, WO 2003043651, WO 2005017160, WO 2011069336, U.S. Pat. Nos. 5,637,323, 7,288,521, 9,782,429, US 2002/0142462, and US 2010/02268.

In particular embodiments, a therapeutically effective amount of G-CSF includes 0.1 μg/kg to 100 μg/kg. In particular embodiments, a therapeutically effective amount of G-CSF includes 0.5 μg/kg to 50 μg/kg. In particular embodiments, a therapeutically effective amount of G-CSF includes 0.5 μg/kg, 1 μg/kg, 2 μg/kg, 3 μg/kg, 4 μg/kg, 5 μg/kg, 6 g/kg, 7 μg/kg, 8 μg/kg, 9 μg/kg, 10 μg/kg, 11 μg/kg, 12 μg/kg, 13 μg/kg, 14 μg/kg, 15 μg/kg, 16 μg/kg, 17 μg/kg, 18 μg/kg, 19 μg/kg, 20 μg/kg, or more. In particular embodiments, a therapeutically effective amount of G-CSF includes 5 μg/kg. In particular embodiments, G-CSF can be administered subcutaneously or intravenously. In particular embodiments, G-CSF can be administered for 1 day, 2 consecutive days, 3 consecutive days, 4 consecutive days, 5 consecutive days, or more. In particular embodiments, G-CSF can be administered for 4 consecutive days. In particular embodiments, G-CSF can be administered for 5 consecutive days. In particular embodiments, as a single agent, G-CSF can be used at a dose of 10 μg/kg subcutaneously daily, initiated 3, 4, 5, 6, 7, or 8 days before adenoviral delivery. In particular embodiments, G-CSF can be administered as a single agent followed by concurrent administration with another mobilization factor. In particular embodiments, G-CSF can be administered as a single agent followed by concurrent administration with AMD3100. In particular embodiments, a treatment protocol includes a 5 day treatment where G-CSF can be administered on day 1, day 2, day 3, and day 4 and on day 5, G-CSF and AMD3100 are administered 6 to 8 hours prior to adenoviral administration.

Therapeutically effective amounts of GM-CSF to administer can include doses ranging from, for example, 0.1 to 50 μg/kg or from 0.5 to 30 μg/kg. In particular embodiments, a dose at which GM-CSF can be administered includes 0.5 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 11 μg/kg, 12 μg/kg, 13 μg/kg. 14 μg/kg, 15 μg/kg, 16 μg/kg, 17 μg/kg, 18 μg/kg, 19 μg/kg, 20 μg/kg, or more. In particular embodiments, GM-CSF can be administered subcutaneously for 1 day, 2 consecutive days, 3 consecutive days, 4 consecutive days, 5 consecutive days, or more. In particular embodiments, GM-CSF can be administered subcutaneously or intravenously. In particular embodiments, GM-CSF can be administered at a dose of 10 μg/kg subcutaneously daily initiated 3, 4, 5, 6, 7, or 8 days before adenoviral delivery. In particular embodiments, GM-CSF can be administered as a single agent followed by concurrent administration with another mobilization factor. In particular embodiments, GM-CSF can be administered as a single agent followed by concurrent administration with AMD3100. In particular embodiments, a treatment protocol includes a 5 day treatment where GM-CSF can be administered on day 1, day 2, day 3, and day 4 and on day 5, GM-CSF and AMD3100 are administered 6 to 8 hours prior to adenoviral administration. A dosing regimen for Sargramostim can include 200 μg/m², 210 μg/m², 220 μg/m², 230 μg/m², 240 μg/m², 250 μg/m², 260 μg/m², 270 μg/m², 280 μg/m², 290 μg/m², 300 μg/m², or more. In particular embodiments, Sargramostim can be administered for 1 day, 2 consecutive days, 3 consecutive days, 4 consecutive days, 5 consecutive days, or more. In particular embodiments, Sargramostim can be administered subcutaneously or intravenously. In particular embodiments, a dosing regimen for Sargramostim can include 250 μg/m²/day intravenous or subcutaneous and can be continued until a targeted cell amount is reached in the peripheral blood or can be continued for 5 days. In particular embodiments, Sargramostim can be administered as a single agent followed by concurrent administration with another mobilization factor. In particular embodiments, Sargramostim can be administered as a single agent followed by concurrent administration with AMD3100. In particular embodiments, a treatment protocol includes a 5 day treatment where Sargramostim can be administered on day 1, day 2, day 3, and day 4 and on day 5, Sargramostim and AMD3100 are administered 6 to 8 hours prior to adenoviral administration.

In particular embodiments, a therapeutically effective amount of AMD3100 includes 0.1 mg/kg to 100 mg/kg. In particular embodiments, a therapeutically effective amount of AMD3100 includes 0.5 mg/kg to 50 mg/kg. In particular embodiments, a therapeutically effective amount of AMD3100 includes 0.5 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, or more. In particular embodiments, a therapeutically effective amount of AMD3100 includes 4 mg/kg. In particular embodiments, a therapeutically effective amount of AMD3100 includes 5 mg/kg. In particular embodiments, a therapeutically effective amount of AMD3100 includes 10 μg/kg to 500 μg/kg or from 50 μg/kg to 400 μg/kg. In particular embodiments, a therapeutically effective amount of AMD3100 includes 100 g/kg, 150 μg/kg, 200 μg/kg, 250 μg/kg, 300 μg/kg, 350 μg/kg, or more. In particular embodiments, AMD3100 can be administered subcutaneously or intravenously. In particular embodiments, AMD3100 can be administered subcutaneously at 160-240 μg/kg 6 to 11 hours prior to adenoviral delivery. In particular embodiments, a therapeutically effective amount of AMID3100 can be administered concurrently with administration of another mobilization factor. In particular embodiments, a therapeutically effective amount of AMD3100 can be administered following administration of another mobilization factor. In particular embodiments, a therapeutically effective amount of AMD3100 can be administered following administration of G-CSF. In particular embodiments, a treatment protocol includes a 5-day treatment where G-CSF is administered on day 1, day 2, day 3, and day 4 and on day 5, G-CSF and AMD3100 are administered 6 to 8 hours prior to adenoviral injection.

Therapeutically effective amounts of SCF to administer can include doses ranging from, for example, 0.1 to 100 μg/kg/day or from 0.5 to 50 μg/kg/day. In particular embodiments, a dose at which SCF can be administered includes 0.5 μg/kg/day, 1 μg/kg/day, 2 μg/kg/day, 3 g/kg/day, 4 μg/kg/day, 5 μg/kg/day, 6 μg/kg/day, 7 μg/kg/day, 8 μg/kg/day, 9 μg/kg/day, 10 μg/kg/day, 11 μg/kg/day, 12 μg/kg/day, 13 μg/kg/day, 14 μg/kg/day, 15 μg/kg/day, 16 μg/kg/day, 17 μg/kg/day, 18 μg/kg/day, 19 μg/kg/day, 20 μg/kg/day, 21 μg/kg/day, 22 μg/kg/day, 23 μg/kg/day, 24 μg/kg/day, 25 μg/kg/day, 26 μg/kg/day, 27 μg/kg/day, 28 μg/kg/day, 29 μg/kg/day, 30 μg/kg/day, or more. In particular embodiments, SCF can be administered for 1 day, 2 consecutive days, 3 consecutive days, 4 consecutive days, 5 consecutive days, or more. In particular embodiments, SCF can be administered subcutaneously or intravenously. In particular embodiments, SCF can be injected subcutaneously at 20 μg/kg/day. In particular embodiments, SCF can be administered as a single agent followed by concurrent administration with another mobilization factor. In particular embodiments, SCF can be administered as a single agent followed by concurrent administration with AMD3100. In particular embodiments, a treatment protocol includes a 5 day treatment where SCF can be administered on day 1, day 2, day 3, and day 4 and on day 5, SCF and AMD3100 are administered 6 to 8 hours prior to adenoviral administration.

In particular embodiments, growth factors GM-CSF and G-CSF can be administered to mobilize HSPCs and/or HSCs in the bone marrow niches to the peripheral circulating blood to increase the fraction of HSPCs and/or HSCs circulating in the blood. In particular embodiments, mobilization can be achieved with administration of G-CSF/Filgrastim (Amgen) and/or AMD3100 (Sigma). In particular embodiments, mobilization can be achieved with administration of GM-CSF/Sargramostim (Amgen) and/or AMD3100 (Sigma). In particular embodiments, mobilization can be achieved with administration of SCF/Ancestim (Amgen) and/or AMD3100 (Sigma). In particular embodiments, administration of G-CSF/Filgrastim precedes administration of AMD3100. In particular embodiments, administration of G-CSF/Filgrastim occurs concurrently with administration of AMD3100. In particular embodiments, administration of G-CSF/Filgrastim precedes administration of AMD3100, followed by concurrent administration of G-CSF/Filgrastim and AMD3100. US 20140193376 describes mobilization protocols utilizing a CXCR4 antagonist with a S1P receptor 1 (S1PR1) modulator agent. US20110044997 describes mobilization protocols utilizing a CXCR4 antagonist with a vascular endothelial growth factor receptor (VEGFR) agonist.

In particular embodiments, administration of vector occurs concurrently with administration of one or more mobilization factors. In particular embodiments, administration of vector follows administration of one or more mobilization factors. In particular embodiments, administration of vector follows administration of a first one or more mobilization factors and occurs concurrently with administration of a second one or more mobilization factors.

In particular embodiments, an HSC enriching agent, such as a CD19 immunotoxin or 5-FU can be administered to enrich for HSPCs and/or HSCs. CD19 immunotoxin can be used to deplete all CD19 lineage cells, which accounts for 30% of bone marrow cells. Depletion encourages exit from the bone marrow. By forcing HSPCs and/or HSCs to proliferate (whether via, e.g., CD19 immunotoxin of 5-FU), this stimulates their differentiation and exit from the bone marrow and increases modified cells in peripheral blood cells.

Therapeutically effective amounts of HSPC and/or HSC mobilization factors, and/or HSPC and/or HSC enriching agents, can be administered through any appropriate administration route such as by, injection, infusion, perfusion, and more particularly by administration by one or more of bone marrow, intravenous, intradermal, intraarterial, intranodal, intralymphatic, intraperitoneal injection, infusion, or perfusion).

In particular embodiments, methods of the present disclosure can include selection for modified cells. In various embodiments, modified cells include cells modified to include a nucleic acid that encodes inhibitor-resistant MGMT. In various embodiments, modified cells include cells modified to include a nucleic acid that encodes inhibitor-resistant MGMT and to include, integrate, and/or express a therapeutic payload. In various embodiments, administration of a selection regimen can select for modified cells (e.g., modified HSCs), eliminate non-modified cells (e.g., non-modified HSCs), and/or contribute to therapeutic efficacy. In various embodiments, selection includes administering a selection regimen to a subject or system including one or more modified cells.

In various embodiments, a selection regimen includes an MGMT inhibitor. In various embodiments, a selection regimen includes an MGMT inhibitor and an alkylating agent. In various embodiments, a selection regimen can include an MGMT inhibitor disclosed herein and an alkylating agent disclosed herein. In various embodiments, a selection regimen includes a single composition or formulation that includes an MGMT inhibitor and an alkylating agent. In various embodiments, a selection regimen includes a first composition or formulation that includes an MGMT inhibitor and at least a second formulation or composition that includes an alkylating agent. In various embodiments, an MGMT inhibitor and an alkylating agent of a selection regimen are administered together (e.g., within the same period of 15, 30, 45, or 60 minutes). In various embodiments, an MGMT inhibitor and an alkylating agent of a selection regimen are administered separately (e.g., at times separated by period of at least 15, 30, 45, or 60 minutes).

In certain particular embodiments, a selection regimen includes O⁶BG or an analog or derivative thereof and an alkylating agent. In certain particular embodiments, a selection regimen includes O⁶BG or an analog or derivative thereof and BCNU. In certain particular embodiments, a selection regimen includes O⁶BG or an analog or derivative thereof and temozolomide. In certain particular embodiments, a selection regimen includes O⁶BG and an alkylating agent. In certain particular embodiments, a selection regimen includes O⁶BG and BCNU. In certain particular embodiments, a selection regimen includes O⁶BG and temozolomide. In certain particular embodiments, a selection regimen includes O⁶-(4-bromothenyl) guanine (O⁶BTG; PaTrin-2) and an alkylating agent. In certain particular embodiments, a selection regimen includes O⁶-(4-bromothenyl) guanine (O⁶BTG; PaTrin-2) and BCNU. In certain particular embodiments, a selection regimen includes O⁶-(4-bromothenyl) guanine (O′BTG; PaTrin-2) and temozolomide.

In certain particular embodiments, a selection regimen includes an MGMT inhibitor and BCNU. In certain particular embodiments, a selection regimen includes an MGMT inhibitor and temozolomide.

In various embodiments, a selection regimen or selection agent thereof is administered together with or concurrently with a vector of the present disclosure to a subject, cell, or system. In various embodiments, a selection regimen or selection agent thereof is administered within 1 hour, 30 minutes, 15 minutes, 10 minutes, 5 minutes, or 1 minute of administration of a vector to a subject, cell, or system

In various embodiments, a selection regimen or selection agent thereof is administered after administration of a vector of the present disclosure to a subject, cell, or system. In certain embodiments, a selection regimen or selection agent thereof is administered at least, up to, or about 1 hour, 2 hours, 3, hours, 4 hours, 5 hours, 6 hours, 12 hours, 1 day, 2 days, 3 days, 4 days, 5 days, 6 days, 1 week, 2 weeks, 3 weeks, 4 weeks, 6 weeks, 8 weeks, 10 weeks, 12 weeks, 14 weeks, 16 weeks, 18 weeks, 20 weeks, 22 weeks, and/or 24 weeks after administration of a vector to a subject, cell, or system.

In various embodiments, a selection regimen or selection agent thereof is administered prior to administration of a vector of the present disclosure to a subject, cell, or system. In certain embodiments, a selection regimen or selection agent thereof is administered at least, up to, or about 1 hour, 2 hours, 3, hours, 4 hours, 5 hours, 6 hours, 12 hours, 1 day, 2 days, 3 days, 4 days, 5 days, 6 days, 1 week, 2 weeks, 3 weeks, 4 weeks, 6 weeks, 8 weeks, 10 weeks, 12 weeks, 14 weeks, 16 weeks, 18 weeks, 20 weeks, 22 weeks, and/or 24 weeks prior to administration of a vector to a subject, cell, or system.

Vectors can be administered concurrently with or following administration of one or more immunosuppression agents or immunosuppression regimens. In various embodiments, one or more immunosuppression agents or immunosuppression regimens are administered after administration of a vector of the present disclosure to a subject, cell, or system. In certain embodiments, one or more immunosuppression agents or immunosuppression regimens are administered at least, up to, or about 1 hour, 2 hours, 3, hours, 4 hours, 5 hours, 6 hours, 12 hours, 1 day, 2 days, 3 days, 4 days, 5 days, 6 days, 1 week, 2 weeks, 3 weeks, 4 weeks, 6 weeks, 8 weeks, 10 weeks, 12 weeks, 14 weeks, 16 weeks, 18 weeks, 20 weeks, 22 weeks, and/or 24 weeks after administration of a vector to a subject, cell, or system. In various embodiments, one or more immunosuppression agents or immunosuppression regimens are administered prior to administration of a vector of the present disclosure to a subject, cell, or system. In certain embodiments, one or more immunosuppression agents or immunosuppression regimens are administered at least, up to, or about 1 hour, 2 hours, 3, hours, 4 hours, 5 hours, 6 hours, 12 hours, 1 day, 2 days, 3 days, 4 days, 5 days, 6 days, 1 week, 2 weeks, 3 weeks, 4 weeks, 6 weeks, 8 weeks, 10 weeks, 12 weeks, 14 weeks, 16 weeks, 18 weeks, 20 weeks, 22 weeks, and/or 24 weeks prior to administration of a vector to a subject, cell, or system.

In vitro gene therapy includes use of a vector to modify a target cell, where the target cell is not present in a multicellular organism (e.g., in a laboratory). In some embodiments, a target cell is derived from a multicellular organism, such as a mammal (e.g., a mouse, rat, human, or non-human primate). In vitro engineering of a cell derived from a multicellular organism can be referred to as ex vivo engineering, and can be used in ex vivo therapy. In various embodiments, methods and compositions of the present disclosure are utilized, e.g., as disclosed herein, to modify a target cell derived from a first multicellular organism and the engineered target cell is then administered to a second multicellular organism, such as a mammal (e.g., a mouse, rat, human, or non-human primate), e.g., in a method of adoptive cell therapy. In some instances, the first and second organisms are the same single subject organism. Return of in vitro engineered material to a subject from which the material was derived can be an autologous therapy. In some instances, the first and second organisms are different organisms (e.g., two organisms of the same species, e.g., two mice, two rats, two humans, or two non-human primates of the same species). Transfer of engineered material derived from a first subject to a second different subject can be an allogeneic therapy. Cells can be autologous or allogeneic in reference to a particular subject. In particular embodiments, the cells are part of an allograft.

Ex vivo cell therapies can include isolation of stem, progenitor or differentiated cells from a patient or a normal donor, expansion of isolated cells ex vivo—with or without genetic engineering—and administration of the cells to a subject to establish a transient or stable graft of the infused cells and/or their progeny. Such ex vivo approaches can be used, for example, to treat an inherited, infectious or neoplastic disease, to regenerate a tissue, or to deliver a therapeutic agent to a subject or disease site. In various ex vivo therapies there is no direct exposure of the subject to the gene transfer vector, and the target cells of transduction can be selected, expanded and/or differentiated, before or after any genetic engineering, to improve efficacy and safety.

Applications of ex vivo therapy include introducing novel nucleic acid sequences and/or functionality. Ex vivo gene therapy can confer a novel function to cells or their progeny. Ex vivo therapies include hematopoietic stem cell (HSC) transplantation (HCT). For example, autologous HSC gene therapy represents a therapeutic option for monogenic diseases of the blood and the immune system as well as for storage disorders. Another established cell and gene therapy application is adoptive immunotherapy, which exploits ex vivo expanded T cells, with or without genetic engineering to redirect their antigen specificity or to increase their safety profile, in order to harness the power of immune effector and regulatory cells for use against malignancies, infections and autoimmune diseases. A range of other types of somatic stem cells can be engineered for therapeutic applications, including epidermal and limbal stem cells, neural stem/progenitor cells (NSPCs), cardiac stem cells and multipotent stromal cells (MSCs).

Applications of ex vivo therapy include reconstituting dysfunctional cell lineages. For inherited diseases characterized by a defective or absent cell lineage, the lineage can be regenerated by functional progenitor cells, derived either from normal donors or from autologous cells that have been subjected to ex vivo modification to correct the deficiency. An example is provided by SCIDs, in which a deficiency in any one of several genes blocks the development of mature lymphoid cells. Transplantation of non-manipulated normal donor HSCs, which can allow generation of donor-derived functional hematopoietic cells of various lineages in the host, represents a therapeutic option for SCIDs, as well as many other diseases that affect the blood and immune system. Autologous HSC gene therapy can include ex vivo modification (e.g., replacing, supplementing, or repairing a defective gene) of HSCs or HSPCs for transplant and, like HCT, can provide a steady supply of functional progeny. Advantages can include reduced risk of graft versus host disease (GvHD), reduced risk of graft rejection, and reduced need for post-transplant immunosuppression.

Applications of ex vivo therapy include enhancing immune responses. For instance, immune cells such as T cells, can be engineered ex vivo to recognize and/or eliminate target cells such as cancer cells. Immune cells such as T cells can be engineered to express, for example, a chimeric antigen receptor that triggers an immune response.

In various embodiments, vectors of the present disclosure can deliver an editing nucleic acid of the present disclosure to a hematopoietic cell. Hematopoietic cell types of the present disclosure include hematopoietic cells of all lineages and stages of hematopoietic cell differentiation. Target cell types of the present disclosure include, without limitation, HSCs (e.g., CD34+ long-term (LT)-HSCs and/or CD34+ short-term (ST)-HSCs), common lymphoid progenitors (CLPs), T cells, NK cells, colony forming unit (CFU)-pre B cells, B cells, common myeloid progenitors (CMPs), granulocyte-macrophage progenitors (GMPs), CFU-M cells, monoblasts, monocytes, macrophages, CFU-G cells, myeloblasts, granulocytes, neutrophils, eosinophils, basophils, megakaryocyte-erythrocyte progenitors (MEPs), BFU-E cells, CFU-E cells, erythroblasts, erythrocytes, CFU-Mk cells, megakaryocytes, and/or platelets. Hematopoietic cell types (e.g., target hematopoietic cell types) of the present disclosure include CD34+ hematopoietic cells.

In various embodiments, vectors of the present disclosure can deliver an editing nucleic acid of the present disclosure to hematopoietic stem cells (HSCs).

In various embodiments, vectors such as certain adenoviral vectors can be targeted to HSCs for in vivo, in vitro, and or ex vivo genetic modification by binding of CD46. HSCs or subsets thereof can also be identified by any of the following marker profiles: CD34+; Lin−/CD34+/CD38−/CD45RA−/CD90+/CD49f+ (HSC1); CD34+/CD38−/CD45RA−/CD90−/CD49f+/(HSC2). In various embodiments, human HSC1 can be identified by any of the following profiles: CD34+/CD38−/CD45RA−/CD90+ or CD34−/CD45RA−/CD90+ and mouse LT-HSC can be identified by Lin−Scal+ckit+CD150+CD48−Flt3−CD34− (where Lin represents the absence of expression of any marker of mature cells including CD3, CD4, CD8, CD11b, CD11c, NK1.1, Gr1, and TER119). In particular embodiments, HSCs are identified by a CD164+ profile. In particular embodiments, HSC are identified by a CD34+/CD164+ profile. For additional information regarding HSC marker profiles, see WO2017/218948. Accordingly, HSCs can be beneficially caused to encode and/or express various payloads and/or agents provided herein, including without limitation MGMT editing payloads, inhibitor-resistant MGMT, and therapeutic payloads.

In various embodiments, hematopoietic cell types that can be targeted by vectors of the present disclosure include T cells. Several different subsets of T-cells have been discovered, each with a distinct function. For example, a majority of T-cells have a T-cell receptor (TCR) existing as a complex of several proteins. The actual T-cell receptor is composed of two separate peptide chains, which are produced from the independent T-cell receptor alpha and beta (TCRα and TCRβ) genes and are called α- and β-TCR chains.

γδ T-cells represent a small subset of T-cells that possess a distinct T-cell receptor (TCR) on their surface. In γδ T-cells, the TCR is made up of one γ-chain and one 8-chain. This group of T-cells is much less common (2% of total T-cells) than the αβ T-cells.

CD3 is expressed on all mature T cells. Activated T-cells express 4-1BB (CD137), CD69, and CD25. CD5 and transferrin receptor are also expressed on T-cells.

T-cells can further be classified into helper cells (CD4+ T-cells) and cytotoxic T-cells (CTLs, CD8+ T-cells), which include cytolytic T-cells. T helper cells assist other white blood cells in immunologic processes, including maturation of B cells into plasma cells and activation of cytotoxic T-cells and macrophages, among other functions. These cells are also known as CD4+ T-cells because they express the CD4 protein on their surface. Helper T-cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T-cells destroy virally infected cells and tumor cells, and are also implicated in transplant rejection. These cells are also known as CD8+ T-cells because they express the CD8 glycoprotein on their surface. These cells recognize their targets by binding to antigen associated with MHC class I, which is present on the surface of nearly every cell of the body.

In particular embodiments, CARs are genetically modified to be expressed in cytotoxic T-cells.

“Central memory” T-cells (or “TCM”) as used herein refers to an antigen experienced CTL that expresses CD62L or CCR7 and CD45RO on the surface thereof, and does not express or has decreased expression of CD45RA as compared to naive cells. In particular embodiments, central memory cells are positive for expression of CD62L, CCR7, CD25, CD127, CD45RO, and CD95, and have decreased expression of CD45RA as compared to naive cells.

“Effector memory” T-cell (or “TEM”) as used herein refers to an antigen experienced T-cell that does not express or has decreased expression of CD62L on the surface thereof as compared to central memory cells and does not express or has decreased expression of CD45RA as compared to a naive cell. In particular embodiments, effector memory cells are negative for expression of CD62L and CCR7, compared to naive cells or central memory cells, and have variable expression of CD28 and CD45RA. Effector T-cells are positive for granzyme B and perforin as compared to memory or naive T-cells.

“Naive” T-cells as used herein refers to a non-antigen experienced T cell that expresses CD62L and CD45RA and does not express CD45RO as compared to central or effector memory cells. In particular embodiments, naive CD8+T lymphocytes are characterized by the expression of phenotypic markers of naive T-cells including CD62L, CCR7, CD28, CD127, and CD45RA.

In various embodiments, hematopoietic cell types that can be targeted by vectors of the present disclosure include B cells. B cells are mediators of the humoral response and are responsible for production and release of antibodies specific to an antigen. Several types of B cells exist which can be characterized by key markers. In general, immature B cells express CD19, CD20, CD34, CD38, and CD45R, and as they mature the key expressed markers are CD19 and IgM.

In various embodiments, hematopoietic cell types that can be targeted by vectors of the present disclosure include natural killer (NK) cells.

In various embodiments, hematopoietic cell types that can be targeted by vectors of the present disclosure include monocytes.

HSCs can differentiate into HSPCs. HSPCs can self-renew or can differentiate into (i) myeloid progenitor cells which ultimately give rise to monocytes and macrophages, neutrophils, basophils, eosinophils, erythrocytes, megakaryocytes/platelets, or dendritic cells; or (ii) lymphoid progenitor cells which ultimately give rise to T-cells, B-cells, and lymphocyte-like cells called natural killer cells (NK-cells). For a general discussion of hematopoiesis and HSPC differentiation, see Chapter 17, Differentiated Cells and the Maintenance of Tissues, Alberts et al., 1989, Molecular Biology of the Cell, 2nd Ed., Garland Publishing, New York, NY; Chapter 2 of Regenerative Medicine, Department of Health and Human Services, Aug. 5, 2006, and Chapter 5 of Hematopoietic Stem Cells, 2009, Stem Cell Information, Department of Health and Human Services.

HSPCs can be positive for a specific marker expressed in increased levels on HSPCs relative to other types of hematopoietic cells. For example, such markers include CD34, CD43, CD45RO, CD45RA, CD59, CD90, CD109, CD117, CD133, CD166, HLA DR, or a combination thereof. Also, the HSPCs can be negative for an expressed marker relative to other types of hematopoietic cells. For example, such markers include Lin, CD38, or a combination thereof. Preferably, HSPCs are CD34+.

HSCs and HSPCs sources include umbilical cord blood, placental blood, bone marrow and peripheral blood (see U.S. Pat. Nos. 5,004,681; 7,399,633; and U.S. Pat. No. 7,147,626; Craddock et al., Blood. 90 (12): 4779-4788 (1997); Jin et al., Bone Marrow Transplant. 42 (9): 581-588 (2008); Jin et al., Bone Marrow Transplant. 42 (7): 455-459 (2008); Pelus, Curr. Opin. Hematol. 15 (4): 285-292 (2008); Papayannopoulou et al., Blood. 91:2231-2239 (1998); Tricot et al., Haematologica. 93 (11): 1739-1742 (2008); and Weaver et al., Bone Marrow Transplant. 27: S23-S29 (2001)), as well as fetal liver, and embryonic stem cells (ESC) and induced pluripotent stem cells (iPSCs) that can be differentiated into HSC. Methods regarding collection, anti-coagulation and processing, etc. of blood and tissue samples are well known in the art. See, for example, Alsever et al., J. Med. 41:126 (1941); De Gowin et al., J. Am. Med. Assoc. 114-850 (1940); Smith et al., J. Thome. Cardiovasc. Surg. 38:573 (1959); Rous and Turner, J. Exp. Med. 23 (2): 219-237 (1916); and Hum, Calif. Med. 108 (3): 218-224 (1968). Stem cell sources of HSCs and HSPCs also include aortal-gonadal-mesonephros derived cells, lymph, liver, thymus, and spleen from age-appropriate donors. All collected stem cell sources of HSCs and HSPCs can be screened for undesirable components and discarded, treated, or used according to accepted current standards at the time. These stem cell sources can be steady state/naïve or primed with mobilizing or growth factor agents.

In order to avoid surgical procedures to perform a bone marrow harvest to isolate HSCs or HSPCs, approaches that harvest stem cells from the peripheral blood can be preferred. Mobilization is a process whereby stem cells are stimulated out of the bone marrow (BM) niche into the peripheral blood (PB), and likely proliferate in the PB. Mobilization allows for a larger frequency of stem cells within the PB minimizing the number of days of apheresis, reaching target number collection of stem cells, and minimizing discomfort to the donor. Agents that enhance mobilization can either enhance proliferation in the PB, or enhance migration from the BM to PB, or both. Various mobilizing agents are described herein and/or known to those of skill in the art. et al.

HSC and/or HSPC can be collected and isolated from a sample using any appropriate technique. Appropriate collection and isolation procedures include magnetic separation; fluorescence activated cell sorting (FACS; Williams et al., Dev. Biol. 112 (1): 126-134, 1985; Lu et al., Exp. Hematol. 14 (10): 955-962, 1986; Lu et al., Blood. 68 (1): 126-133, 1986); nanosorting based on fluorophore expression; affinity chromatography; cytotoxic agents joined to a monoclonal antibody or used in conjunction with a monoclonal antibody, e.g., complement and cytotoxins; “panning” with an antibody attached to a solid matrix; selective agglutination using a lectin such as soybean (Reisner et al., Lancet. 2 (8208-8209): 1320-1324, 1980); immunomagnetic bead-based sorting or combinations of these techniques, etc. These techniques can also be used to assay for successful engraftment or manipulation of hematopoietic cells in vivo, for example for gene transfer, genetic editing or cell population expansion.

In particular embodiments, it is important to remove contaminating cell populations that would interfere with isolation of the intended cell population, such as red blood cells. Removing includes both biochemical and mechanical methods to remove the undesired cell populations. Examples include lysis of red blood cells using detergents, hetastarch (hydroxyethyl starch), hetastarch with centrifugation, cell washing, cell washing with density gradient, Ficoll-hypaque, Sepx, Optipress, filters, and other protocols that have been used both in the manufacture of HSC and/or gene therapies for research and therapeutic purposes.

In particular embodiments, a sample can be processed to select/enrich for CD34+ cells using anti-CD34 antibodies directly or indirectly conjugated to magnetic particles in connection with a magnetic cell separator, for example, the CliniMACS® Cell Separation System (Miltenyi Biotec, Bergisch Gladbach, Germany). See also, sec. 5.4.1.1 of U.S. Pat. No. 7,399,633 which describes enrichment of CD34+ HSC/HSPC from 1-2% of a normal bone marrow cell population to 50-80% of the population. HSC can also be selected to achieve the HSC profiles noted above, such as CD34+/CD45RA−/CD90+ or CD34+/CD38−/CD45RA−/CD90+.

Similarly, HSPC expressing CD43, CD45RO, CD45RA, CD59, CD90, CD109, CD117, CD133, CD166, HLA DR, or a combination thereof, can be enriched for using antibodies against these antigens. U.S. Pat. No. 5,877,299 describes additional appropriate hematopoietic antigens that can be used to isolate, collect, and enrich HSPC cells from samples.

Following isolation and/or enrichment, HSC or HSPC can be expanded in order to increase the number of HSC/HSPC. Isolation and/or expansion methods are described in, for example, U.S. Pat. Nos. 7,399,633 and 5,004,681; US Patent Publication No. 2010/0183564; International Patent Publications No. WO 2006/047569; WO 2007/095594; WO 2011/127470; and WO 2011/127472; Varnum-Finney et al., Blood 101:1784-1789, 1993; Delaney et al., Blood 106:2693-2699, 2005; Ohishi et al., J. Clin. Invest. 110:1165-1174, 2002; Delaney et al., Nature Med. 16 (2): 232-236, 2010; and Chapter 2 of Regenerative Medicine, Department of Health and Human Services, August 2006, and the references cited therein. Each of the referenced methods of collection, isolation, and expansion can be used in particular embodiments of the disclosure.

Particular methods of expanding HSC/HSPC include expansion with a Notch agonist. For information regarding expansion of HSC/HSPC using Notch agonists, see sec. 5.1 and 5.3 of U.S. Pat. Nos. 7,399,633; 5,780,300; 5,648,464; 5,849,869; and 5,856,441; WO 1992/119734; Schlondorfiand & Blobel, J. Cell Sci. 112:3603-3617, 1999; Olkkonen and Stenmark, Int. Rev. Cytol. 176:1-85, 1997; Kopan et al., Cell 137:216-233, 2009; Rebay et al., Cell 67:687-699, 1991 and Jarriault et al., Mol. Cell. Biol. 18:7423-7431, 1998.

Additional culture conditions can include expansion in the presence of one or more growth factors, such as: angiopoietin-like proteins (Angptls, e.g., Angptl2, Angptl3, Angptl7, Angpt15, and Mfap4); erythropoietin; fibroblast growth factor-1 (FGF-1); Flt-3 ligand (Flt-3L); G-CSF; GM-CSF; insulin growth factor-2 (IGF-2); interleukin-3 (IL-3); interleukin-6 (IL-6); interleukin-7 (IL-7); interleukin-11 (IL-11); stem cell factor (SCF; also known as the c-kit ligand or mast cell growth factor); thrombopoietin (TPO); and analogs thereof (wherein the analogs include any structural variants of the growth factors having the biological activity of the naturally occurring growth factor; see, e.g., WO 2007/1145227 and U.S. Patent Publication No. 2010/0183564). As a particular example for expanding HSC/HSPC, the cells can be cultured on a plastic tissue culture dish containing immobilized Delta ligand and fibronectin and 50 ng/ml of each of SCF, Flt-3L and TPO.

### Conditions Treatable by Gene Therapy

At least in part because vectors of the present disclosure (e.g., adenoviral vectors) can be used in vivo, in vitro, or ex vivo for modification of host and/or target cells, and further because a vector can include payloads encoding a wide variety of expression products, it will be clear from the present specification that various technologies provided herein have broad applicability and can be used to treat a wide variety of conditions. Examples of conditions treatable by administration of adenoviral vector, genome, or system of the present disclosure include, without limitation, genetic conditions (e.g., hemoglobinopathies) and conditions treatable by expression of a therapeutic polypeptide (e.g., cancer).

In various embodiments, methods and compositions of the present disclosure can be used to treat a genetic condition (e.g., a condition arising from and/or caused by a mutation present in the genome of one or more cells of a subject). In various embodiments, methods and compositions of the present disclosure can be used to treat a genetic condition arising from and/or caused by a single point mutation present in the genome of one or more cells of a subject (e.g., a heterozygous or homozygous single point mutation). In various embodiments, methods and compositions of the present disclosure can be used to treat a protein deficiency. In various embodiments, methods and compositions of the present disclosure can be used to treat an enzyme deficiency. In various embodiments, methods and compositions of the present disclosure can be used to treat a blood condition (e.g., a condition characterized by a blood cell abnormality). Examples of genetic (e.g., point mutation) conditions, protein deficiencies, enzyme deficiencies, and/or blood conditions that can be treated by methods and compositions of the present disclosure include adenosine deaminase deficiency (ADA), adrenoleukodystrophy (ALD), agammaglobulinemia, alpha-1 antitrypsin deficiency, congenital amegakaryocytic thrombocytopenia, amyotrophic lateral sclerosis (Lou Gehrig's disease), ataxia telangiectasia, Batten disease, Bernard-Soulier Syndrome, CD40/CD40L deficiency, chronic granulomatous disease, common variable immune deficiency (CVID), congenital thrombotic thrombocytopeni purpura (cTTP), cystic fibrosis, Diamond Blackfan anemia (DBA), DOCK 8 deficiency, dyskeratosis congenital, Fabry disease, Factor V Deficiency, Factor VII Deficiency, Factor X Deficiency, Factor XI Deficiency, Factor XII Deficiency, Factor XIII Deficiency, familial apolipoprotein E deficiency and atherosclerosis (ApoE), familial erythrophagocytic lymphohistiocytosis, Fanconi anemia (FA), Friedreich ataxia, Gaucher disease, Glanzmann thrombasthenia, glucosemia, glycogen storage disease, glycogen storage disease type I (GSDI), Gray Platelet Syndrome, hemophilia, hemophilia A, hemophilia B, hereditary hemochromatosis, Hurler's syndrome, hyper IgM, Hypogammaglobulinemia, Krabbe disease, major histocompatibility complex class II deficiency (MHC-II), maple syrup urine disease, metachromatic leukodystrophy (MLD), mucopolysaccharidoses, mucopolysaccharidosis type I (MPS I), MPS II (Hunter Syndrome), MPS III (Sanfilippo syndrome), MPS IV (Morquio syndrome), MPS V, MPS VI (Maroteaux-Lamy syndrome), MPS VII (sly syndrome), muscular dystrophy, Niemann-Pick disease, Parkinson's disease, paroxysmal nocturnal hemoglobinuria (PNH), pernicious anemia, phenylketonuria (PKU), Pompe disease, pulmonary alveolar proteinosis (PAP), pure red cell aplasia (PRCA), pyruvate kinase deficiency, refractory anemia, Shwachman-Diamond syndrome, selective IgA deficiency, severe aplastic anemia, severe combined immunodeficiency disease (SCID), Severe combined immunodeficiency due to adenosine deaminase deficiency (ADA-SCID), sickle cell anemia, sickle cell disease, sickle cell trait, Tay Sachs, thalassemia, thalassemia intermedia, von Gierke disease, von Willebrand Disease, Wiskott-Aldrich syndrome (WAS), X-linked agammaglobulinemia (XLA), X-linked severe combined immunodeficiency (SCID-X1), Zellweger syndrome, α-mannosidosis, β-mannosidosis, and/or β-thalassemia, β-thalassemia major.

In various embodiments, methods and compositions of the present disclosure can be used to treat an inborn error of metabolism. In various embodiments, methods and compositions of the present disclosure can be used to treat a hyperproliferative condition.

In various embodiments, methods and compositions of the present disclosure can be used to treat a cancer (e.g., a cancer characterized by abnormal blood cells). Examples of cancers that can be treated by methods and compositions of the present disclosure include acute lymphoblastic leukemia (ALL), acute myelogenous leukemia (AML), agnogenic myeloid metaplasia, astrocytoma, atypical teratoid rhabdoid tumor, brain and central nervous system (CNS) cancer, breast cancer, carcinosarcoma, chondrosarcoma, chordoma, choroid plexus carcinoma, choroid plexus papilloma, chronic lymphocytic leukemia (CLL), chronic myelogenous leukemia (CML), clear cell sarcoma of soft tissue, diffuse large B-cell lymphoma, ependymoma, epithelioid sarcoma, Ewing sarcoma, extragonadal germ cell tumor, extrarenal rhabdoid tumor, follicular lymphoma, gastrointestinal stromal tumor, glioblastoma, HBV-induced hepatocellular carcinoma, head and neck cancer, Hodgkin's lymphoma, juvenile myelomonocytic leukemia, kidney cancer, lung cancer, lymphoma, malignant rhabdoid tumor, medulloblastoma, melanoma, meningioma, mesothelioma, multiple myeloma, myeloma, neuroglial tumor, non-Hodgkin's lymphoma, not otherwise specified (NOS) sarcoma, oligoastrocytoma, oligodendroglioma, osteosarcoma, ovarian cancer, ovarian clear cell adenocarcinoma, ovarian endometrioid adenocarcinoma, ovarian serous adenocarcinoma, pancreatic cancer, pancreatic ductal adenocarcinoma, pancreatic endocrine tumor, pineoblastoma, prostate cancer, renal cell carcinoma, renal medullary carcinoma, rhabdomyosarcoma, sarcoma, schwannoma, skin squamous cell carcinoma, and/or stem cell cancer.

In various embodiments, methods and compositions of the present disclosure can be used to treat a hemoglobinopathy, red blood cell disorder, platelet disorder, and/or bone marrow disorder (e.g., a bone marrow failure condition).

In various embodiments, methods and compositions of the present disclosure can be used to treat an immune condition (e.g., an autoimmune condition). Examples of immune conditions (e.g., autoimmune conditions) that can be treated by methods and compositions of the present disclosure include acquired immunodeficiency syndrome (AIDS), acquired thrombotic thrombocytopenia purpura (aTTP), an autoimmune hematology, graft versus host disease (GVHD), Grave's Disease, inflammatory bowel disease, Multiple Sclerosis (MS), rheumatoid arthritis, severe aplastic anemia, and systemic lupus erythematosus (SLE).

In various embodiments, methods and compositions of the present disclosure can be used to treat an immunodeficiency (e.g., a primary immune deficiency, secondary immune deficiency, acquired immune deficiency, and/or an immune deficiency caused by trauma), an inflammatory condition, an IgG subclass deficiency, a complement disorders, or a specific antibody deficiency). In various embodiments, methods and compositions of the present disclosure can be used to eliminate or inhibit one or more subsets of lymphocytes (e.g., induce apoptosis in lymphocytes, inhibit lymphocyte activation, inhibit T cell activation, and/or inhibit Th-2 activity, and/or Th-1 activity), eliminate or inhibit autoreactive T cells, improve kinetics and/or clonal diversity of lymphocyte reconstitution, restore normal T lymphocyte development, restore thymic output, induce selective tolerance to an inciting agent, provide function to immune and other blood cells or treat an immune-mediated condition, In various embodiments, methods and compositions of the present disclosure can be used to normalize primary and secondary antibody responses to immunization.

In various embodiments, methods and compositions of the present disclosure can be used to treat and/or prevent an infection. In various embodiments, a composition of the present disclosure is a vaccine in that it encodes, and/or expresses in one or more cells of a subject, an antigen characteristic of an infectious agent (e.g., a viral or bacterial pathogen). In various embodiments, a method of the present disclosure is a method of vaccination in that it delivers to one or more cells of a subject an antigen characteristic of an infectious agent (e.g., a viral or bacterial pathogen) and/or induces an immune responses against the antigen and/or infectious agent. In various embodiments, a method or composition of the present disclosure delivers (e.g., causes transient expression of) an antigen in a subject. In various embodiments, a method or composition of the present disclosure is used to treat a subject that has the infection. In various embodiments, a method or composition of the present disclosure is used to treat a subject that is at risk of infection. In particular embodiments, the infectious disease is human immunodeficiency virus (HIV). A payload expression product can be, for example, an agent that renders a subject resistant to HIV infection, or which enables immune cells to effectively neutralize HIV. A therapeutically effective amount for the treatment of HIV, for example, may increase the immunity of a subject against HIV, ameliorate a symptom associated with AIDS or HIV, or induce an innate or adaptive immune response in a subject against HIV. An immune response against HIV may include antibody production and result in the prevention of AIDS and/or ameliorate a symptom of AIDS or HIV infection of the subject, or decrease or eliminate HIV infectivity and/or virulence.

In various embodiments, a method or composition of the present disclosure delivers to one or more cells of a subject in need thereof a coding sequence that encodes and/or expresses a replacement polypeptide (i.e., a wild type, reference, and/or functional polypeptide that corresponds to a disease variant encoded by the genome of the subject). In various embodiments, a method or composition of the present disclosure delivers to one or more cells of a subject in need thereof an editing system that modifies a nucleic acid of the subject (e.g., a genome of the subject) to express and/or increase expression of a wild type, reference, and/or functional polypeptide, e.g., by correction of a disease mutation present in the nucleic acid of the subject.

Particular examples of conditions that can be treated by methods and compositions of the present disclosure include conditions in which mutations of a globin gene results in expression of an abnormal form of hemoglobin (e.g., as in sickle cell disease (SCD) and hemoglobin C, D, and E disease) or results in reduced production of the a or B polypeptides (and thus an imbalance of the globin chains in the cell). These latter conditions are termed α- or β-thalassemias, depending on which globin chain is impaired. 5% of the world population carries a significant hemoglobin variant with the sickle cell mutation in the b-globin (HBB) gene (a glutamate to valine conversion; historically E6V, contemporaneously E7V) being by far the most common (40% of carriers). The high prevalence and severity of hemoglobin disorders presents a substantial burden, impacting not only the lives of those affected but also health-care systems, since lifelong patient care is costly.

There are two forms of hemoglobin, fetal (HbF), which includes two alpha (a) and two gamma (γ) chains (see e.g., PDB: 4MQJ_E for the alpha chain; and PDB: 4MQJ_F for the gamma chain), and adult (HbA), which includes two a and two beta (B) chains (see e.g., UniProtKB/Swiss-Prot: P69905.2 for the alpha chain; and UniProtKB/Swiss-Prot: P68871.2 for the beta chain). The natural switch from HbF to HbA occurs shortly after birth and is regulated by transcriptional repression of γ globin genes by factors including a master regulator, bcl11a. Critically, a variety of clinical observations demonstrate that the severity of β-hemoglobinopathies such as sickle cell disease and β-thalassemia are ameliorated by increased production of HbF.

In particular embodiments, a therapeutically effective treatment induces or increases expression of HbF, induces or increases production of hemoglobin, and/or induces or increases production of β-globin. In particular embodiments, a therapeutically effective treatment improves blood cell function, and/or increases oxygenation of cells.

In various embodiments, the present disclosure includes treatment of a blood disorder using a vector of the present disclosure that includes a coding nucleic acid sequence that encodes a protein or agent for treatment of the blood disorder. In various embodiments, the blood disorder is thalassemia and the protein is a β-globin or γ-globin protein, or a protein that otherwise partially or completely functionally replaces β-globin or γ-globin. In various embodiments, the blood disorder is hemophilia and the protein is ET3 or a protein that otherwise partially or completely functionally replaces Factor VIII. In various embodiments, the blood disorder is a point mutation disease such as sickle cell anemia, and the agent is a gene editing protein

ET3 can have or include the following amino acid sequence: SEQ ID NO: 219. In various embodiments, a Factor VIII replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 219

(MQLELSTCVFLCLLPLGFSAIRRYYLGAVELSWDYRQSELLREL
HVDTRFPATAPGALPLGPSVLYKKTVFVEFTDQLFSVARPRPPWM
GLLGPTIQAEVYDTVVVTLKNMASHPVSLHAVGVSFWKSSEGAEY
EDHTSQREKEDDKVLPGKSQTYVWQVLKENGPTASDPPCLTYSYL
SHVDLVKDLNSGLIGALLVCREGSLTRERTQNLHEFVLLFAVFDE
GKSWHSARNDSWTRAMDPAPARAQPAMHTVNGYVNRSLPGLIGCH
KKSVYWHVIGMGTSPEVHSIFLEGHTFLVRHHRQASLEISPLTFL
TAQTFLMDLGQFLLFCHISSHHHGGMEAHVRVESCAEEPQLRRK
ADEEEDYDDNLYDSDMDVVRLDGDDVSPFIQIRSVAKKHPKTWVH
YIAAEEEDWDYAPLVLAPDDRSYKSQYLNNGPQRIGRKYKKVRFM
AYTDETFKTREAIQHESGILGPLLYGEVGDTLLIIFKNQASRPYN
IYPHGITDVRPLYSRRLPKGVKHLKDFPILPGEIFKYKWTVTVED
GPTKSDPRCLTRYYSSFVNMERDLASGLIGPLLICYKESVDQRGN
QIMSDKRNVILFSVFDENRSWYLTENIQRFLPNPAGVQLEDPEFQ
ASNIMHSINGYVFDSLQLSVCLHEVAYWYILSIGAQTDFLSVFFS
GYTFKHKMVYEDTLTLFPFSGETVFMSMENPGLWILGCHNSDFRN
RGMTALLKVSSCDKNTGDYYEDSYEDISAYLLSKNNAIEPRSFAQ
NSRPPSASAPKPPVLRRHQRDISLPTFQPEEDKMDYDDIFSTETK
GEDFDIYGEDENQDPRSFQKRTRHYFIAAVEQLWDYGMSESPRAL
RNRAQNGEVPRFKKVVFREFADGSFTQPSYRGELNKHLGLLGPYI
RAEVEDNIMVTFKNQASRPYSFYSSLISYPDDQEQGAEPRHNFVQ
PNETRTYFWKVQHHMAPTEDEFDCKAWAYFSDVDLEKDVHSGLIG
PLLICRANTLNAAHGRQVTVQEFALFFTIFDETKSWYFTENVERN
CRAPCHLQMEDPTLKENYRFHAINGYVMDTLPGLVMAQNQRIRWY
LLSMGSNENIHSIHFSGHVFSVRKKEEYKMAVYNLYPGVFETVEM
LPSKVGIWRIECLIGEHLQAGMSTTFLVYSKKCQTPLGMASGHIR
DFQITASGQYGQWAPKLARLHYSGSINAWSTKEPFSWIKVDLLAP
MIIHGIKTQGARQKFSSLYISQFIIMYSLDGKKWQTYRGNSTGTL
MVFFGNVDSSGIKHNIFNPPIIARYIRLHPTHYSIRSTLRMELM
GCDLNSCSMPLGMESKAISDAQITASSYFTNMFATWSPSKARLHL
QGRSNAWRPQVNNPKEWLQVDFQKTMKVTGVTTQGVKSLLTSMYV
KEFLISSSQDGHQWTLFFQNGKVKVFQGNQDSFTPVVNSLDPPLL
TRYLRIHPQSWVHQIALRMEVLGCEAQDLYV).

β-globin can have or include the following amino acid sequence: SEQ ID NO: 220. In various embodiments, a β-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 220

(MVHLTPEEKSAVTALWGKVNVDEVGGEALGRLLVVYPWTQRFFE
SFGDLSTPDAVMGNPKVKAHGKKVLGAFSDGLAHLDNLKGTFATL
SELHCDKLHVDPENFRLLGNVLVCVLAHHFGKEFTPPVQAAYQKV
VAGVANALAHKYH).

γ-globin can have or include the following amino acid sequence: SEQ ID NO: 221. In various embodiments, a γ-globin replacement protein can have an amino acid sequence having at least 70%, 75%, 80%, 85%, 90%, 91%, 92%, 93%, 94%, 95%, 96%, 97%, 98%, 99%, or 100% identity to SEQ ID NO: 221

(MGHFTEEDKATITSLWGKVNVEDAGGETLGRLLVVYPWTQRFFD
SFGNLSSASAIMGNPKVKAHGKKVLTSLGDATKHLDDLKGTFAQL
SELHCDKLHVDPENFKLLGNVLVTVLAIHFGKEFTPEVQASWQKM
VTAVASALSSRYH).

In various embodiments, gene therapy of the present disclosure can include and/or be directed to a modification as disclosed in WO 2021/003432, which is herein incorporated by reference in its entirety, and particularly with respect to therapeutic gene modifications.

### Dosages, Formulations, and Administration

A vector and/or nucleic acid for delivery of an editing nucleic acid of the present disclosure can be formulated such that it is acceptable (e.g., pharmaceutically acceptable) for administration to cells or animals, e.g., to humans. A vector and/or nucleic acid for delivery of an editing nucleic acid of the present disclosure may be administered in vitro, ex vivo, or in vivo. Vectors described herein can be formulated for administration to a subject. Formulations can include one or more pharmaceutically acceptable carriers.

As disclosed herein, a vector and/or nucleic acid for delivery of an editing nucleic acid of the present disclosure can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a vector can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intracisternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a vector and/or nucleic acid for delivery of an editing nucleic acid of the present disclosure of the present invention can be formulated as a solution, microemulsion, dispersion, liposome, lipid nanoparticle, or other ordered structure suitable for delivery to a subject, cell, or system, and/or stable storage at high concentration. Sterile injectable solutions can be prepared by incorporating a composition described herein in a pharmaceutically appropriate amount in a pharmaceutically appropriate solvent with one or a combination of ingredients enumerated above, as required, followed by filter sterilization. Generally, dispersions can be prepared by incorporating a composition described herein into a sterile vehicle that contains a basic dispersion medium and other ingredients as needed, e.g., from those enumerated above. In the case of sterile powders for the preparation of sterile injectable solutions, methods for preparation include vacuum drying and freeze-drying that yield a powder of a composition described herein plus any additional desired ingredient from a previously sterile-filtered solution thereof. The proper fluidity of a solution can be maintained, for example, by the use of a coating such as lecithin, by the maintenance of the required particle size in the case of dispersion, and/or by the use of surfactants. Prolonged absorption of injectable compositions can be brought about by including in the composition a reagent that delays absorption, for example, monostearate salts and/or gelatin.

A vector can be administered parenterally in the form of an injectable formulation including a sterile solution or suspension in water or another pharmaceutically acceptable liquid. For example, the vector can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of vector included in the pharmaceutical preparations is such that a suitable dose within the designated range is provided. Examples of oils include sesame oil and soybean oil, optionally combined with benzyl benzoate or benzyl alcohol as a solubilizing agent. Other items that may be included are a buffer such as a phosphate buffer or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a vector described herein can be therapeutically delivered to a subject by way of local administration. As used herein, “local administration” or “local delivery,” can refer to delivery that does not rely upon transport of the vector or vector to its intended target tissue or site via the vascular system. For example, the vector may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, compositions provided herein are present in unit dosage form, which unit dosage form can be suitable for self-administration. Such a unit dosage form may be provided within a container, typically, for example, a vial, cartridge, prefilled syringe or disposable pen. A doser may also be used, for example, with an injection system as described herein.

Pharmaceutical forms of vector formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Isotonic agents (e.g., sugars or sodium chloride) can be used in the formulations.

A suitable dose of a vector described herein can depend on a variety of factors including, e.g., the age, sex, and weight of a subject to be treated, the condition or disease to be treated, and the particular vector used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a vector can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

In various instances, a vector can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

In various embodiments, a composition including a vector as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80, HCO-50 and the like.

The formulations disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

Therapeutically effective amounts of a viral vector can include doses ranging from, for example, 1×10⁷ to 50×10⁸ infection units (IU) or from 5×10⁷ to 20×10⁸ IU. In other examples, a dose can include 5×10⁷ IU, 6×10⁷ IU, 7×10⁷ IU, 8×10⁷ IU, 9×10⁷ IU, 1×10⁸ IU, 2×10⁸ IU, 3×10⁸ IU, 4×10⁸ IU, 5×10⁸ IU, 6×10⁸ IU, 7×10⁸ IU, 8×10⁸ IU, 9×10⁸ IU, 10×10⁸ IU, or more. In particular embodiments, a therapeutically effective amount of viral vector includes 4×10⁸ IU. In particular embodiments, a therapeutically effective amount of viral vector can be administered subcutaneously or intravenously. In particular embodiments, a therapeutically effective amount of viral vector associated with a therapeutic gene and/or expression product can be administered following administration with one or more mobilization factors.

In various embodiments of the present disclosure, an in vivo gene therapy includes administration of at least one viral vector to a subject in combination with at least one immune suppression regimen.

In an in vivo, in vitro, and or ex vivo gene therapy including more than one vector species, such as a first vector that is a viral vector in combination with a second vector that is a support vector, the first vector and the second vector can be administered in a single formulation or dosage form or in two separate formulations or dosage forms. In various embodiments, the first and second vectors can be administered at the same time or at different times, e.g., during the same one-hour period or during non-overlapping one-hour periods. In various embodiments, the first and second vectors can be administered at the same time or at different times, e.g., on the same day or on different days. In various embodiments, the first and second vectors can be administered at the same dosage or at different dosages, e.g., where the dosage is measured as the total number of viral particles or as a number of viral particles per kilogram of the subject. In various embodiments, the first and second vectors can be administered in a pre-defined ratio. In various embodiments, the ratio is in the range of 2:1 to 1:2, e.g., 1:1.

In various embodiments, a vector is administered to a subject in a single total dose on a single day. In various embodiments, a vector is administered in two, three, four, or more unit doses that together constitute a total dose. In various embodiments, one unit dose of a vector is administered to a subject per day on each of one, two, three, four, or more consecutive days. In various embodiments, two unit doses of a vector are administered to a subject per day on each of one, two, three, four, or more consecutive days. Accordingly, in various embodiments, a daily dose can refer to the dose of vector received by a subject over the course of a day. In various embodiments, the term day refers to a twenty-four-hour period, such as a twenty-four-hour period from midnight of a first calendar date to midnight of the next calendar date.

In various embodiments, a unit dose, daily dose, or total dose of a vector, such as a viral vector, or the total combined dose of a viral vector and a support vector, can be at least 1E8, 5E8, 1E9, 5E9, 1E10, 5E10, 1E11, 5E11, 1E12, 5E12, 1E13, 5E13, 1E14, or 1E15 viral particles per kilogram (vp/kg). In various embodiments, a unit dose, daily dose, or total dose of a vector, such as a viral vector, or the total combined dose of a viral vector and a support vector, can fall within a range having a lower bound selected from 1E8, 5E8, 1E9, 5E9, 1E10, 5E10, 1E11, 5E11, 1E12, 5E12, 1E13, 5E13, 1E14, or 1E15 vp/kg and an upper bound selected from 1E8, 5E8, 1E9, 5E9, 1E10, 5E10, 1E11, 5E11, 1E12, 5E12, 1E13, 5E13, 1E14, or 1E15 vp/kg.

### Selection Agent Dosages, Formulations, and Administration

A selection agent (e.g., an agent including an MGMT inhibitor, an alkylating agent, or a combination thereof) of the present disclosure can be formulated such that it is pharmaceutically acceptable for administration to cells or animals, e.g., to humans. A selection agent may be administered in vitro, ex vivo, or in vivo. Selection agents described herein can be formulated for administration to a subject. Formulations can include one or more pharmaceutically acceptable carriers.

As disclosed herein, a selection agent can be in any form known in the art. Such forms include, e.g., liquid, semi-solid and solid dosage forms, such as liquid solutions (e.g., injectable and infusible solutions), dispersions or suspensions, tablets, pills, powders, liposomes and suppositories.

Selection or use of any particular form may depend, in part, on the intended mode of administration and therapeutic application. For example, compositions containing a composition intended for systemic or local delivery can be in the form of injectable or infusible solutions. Accordingly, a selection agent can be formulated for administration by a parenteral mode (e.g., intravenous, subcutaneous, intraperitoneal, or intramuscular injection). As used herein, parenteral administration refers to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intracisternal injection and infusion. A parenteral route of administration can be, for example, administration by injection, transnasal administration, transpulmonary administration, or transcutaneous administration. Administration can be systemic or local by intravenous injection, intramuscular injection, intraperitoneal injection, subcutaneous injection.

In various embodiments, a selection agent of the present invention can be formulated as a solution, microemulsion, dispersion, liposome, lipid nanoparticle, or other ordered structure suitable for delivery to a subject, cell, or system, and/or stable storage at high concentration. Sterile injectable solutions can be prepared by incorporating a composition described herein in a pharmaceutically appropriate amount in a pharmaceutically appropriate solvent with one or a combination of ingredients enumerated above, as required, followed by filter sterilization. Generally, dispersions can be prepared by incorporating a composition described herein into a sterile vehicle that contains a basic dispersion medium and other ingredients as needed, e.g., from those enumerated above. In the case of sterile powders for the preparation of sterile injectable solutions, methods for preparation include vacuum drying and freeze-drying that yield a powder of a composition described herein plus any additional desired ingredient from a previously sterile-filtered solution thereof. The proper fluidity of a solution can be maintained, for example, by the use of a coating such as lecithin, by the maintenance of the required particle size in the case of dispersion, and/or by the use of surfactants. Prolonged absorption of injectable compositions can be brought about by including in the composition a reagent that delays absorption, for example, monostearate salts and/or gelatin.

A selection agent can be administered parenterally in the form of an injectable formulation including a sterile solution or suspension in water or another pharmaceutically acceptable liquid. For example, the selection agent can be formulated by suitably combining the therapeutic molecule with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of selection agent included in the pharmaceutical preparations is such that a suitable dose within the designated range is provided. Examples of oils include sesame oil and soybean oil, optionally combined with benzyl benzoate or benzyl alcohol as a solubilizing agent. Other items that may be included are a buffer such as a phosphate buffer or sodium acetate buffer, a soothing agent such as procaine hydrochloride, a stabilizer such as benzyl alcohol or phenol, and an antioxidant. The formulated injection can be packaged in a suitable ampule.

In various embodiments, subcutaneous administration can be accomplished by means of a device, such as a syringe, a prefilled syringe, an auto-injector (e.g., disposable or reusable), a pen injector, a patch injector, a wearable injector, an ambulatory syringe infusion pump with subcutaneous infusion sets, or other device for subcutaneous injection.

In some embodiments, a selection agent described herein can be therapeutically delivered to a subject by way of local administration. As used herein, “local administration” or “local delivery,” can refer to delivery that does not rely upon transport of the selection agent to its intended target tissue or site via the vascular system. For example, the selection agent may be delivered by injection or implantation of the composition or agent or by injection or implantation of a device containing the composition or agent. In certain embodiments, following local administration in the vicinity of a target tissue or site, the composition or agent, or one or more components thereof, may diffuse to an intended target tissue or site that is not the site of administration.

In some embodiments, compositions provided herein are present in unit dosage form, which unit dosage form can be suitable for self-administration. Such a unit dosage form may be provided within a container, typically, for example, a vial, cartridge, prefilled syringe or disposable pen. A doser may also be used, for example, with an injection system as described herein.

Pharmaceutical forms of selection agent formulations suitable for injection can include sterile aqueous solutions or dispersions. A formulation can be sterile and must be fluid to allow proper flow in and out of a syringe. A formulation can also be stable under the conditions of manufacture and storage. A carrier can be a solvent or dispersion medium containing, for example, water and saline or buffered aqueous solutions. Isotonic agents (e.g., sugars or sodium chloride) can be used in the formulations.

A suitable dose of a selection agent described herein can depend on a variety of factors including, e.g., the age, sex, and weight of a subject to be treated, the condition or disease to be treated, and the particular selection agent used. Other factors affecting the dose administered to the subject include, e.g., the type or severity of the condition or disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. A suitable means of administration of a selection agent can be selected based on the condition or disease to be treated and upon the age and condition of a subject. Dose and method of administration can vary depending on the weight, age, condition, and the like of a patient, and can be suitably selected as needed by those skilled in the art. A specific dosage and treatment regimen for any particular subject can be adjusted based on the judgment of a medical practitioner.

In various instances, a selection agent can be formulated to include a pharmaceutically acceptable carrier or excipient. Examples of pharmaceutically acceptable carriers include, without limitation, any and all solvents, dispersion media, coatings, antibacterial and antifungal agents, isotonic and absorption delaying agents, and the like that are physiologically compatible. Compositions of the present invention can include a pharmaceutically acceptable salt, e.g., an acid addition salt or a base addition salt.

Exemplary generally used pharmaceutically acceptable carriers include any and all absorption delaying agents, antioxidants, binders, buffering agents, bulking agents or fillers, chelating agents, coatings, disintegration agents, dispersion media, gels, isotonic agents, lubricants, preservatives, salts, solvents or co-solvents, stabilizers, surfactants, and/or delivery vehicles.

In various embodiments, a composition including a selection agent as described herein, e.g., a sterile formulation for injection, can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

The formulations disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). The aqueous solutions can include formulatory agents such as suspending, stabilizing, and/or dispersing agents. Alternatively, the formulation can be in lyophilized and/or powder form for constitution with a suitable vehicle, e.g., sterile pyrogen-free water, before use.

Any formulation disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by US FDA Office of Biological Standards and/or other relevant foreign regulatory agencies.

Therapeutically effective amounts of a selection agent can include a dose of an MGMT inhibitor such as O⁶BG or an analog or derivative thereof that ranges from, for example, 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). In particular embodiments, a therapeutically effective amount of MGMT inhibitor includes 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). Therapeutically effective amounts of a selection agent can include a dose of an alkylating agent such as BCNU or an analog or derivative thereof that ranges from, for example, 0.001 to 100 mg/kg (e.g., 1-5, 1-10, 1-20, 1-50, 5-10, 5-20, or 5-50 mg/kg). Therapeutically effective amounts of a selection agent can include a dose of an alkylating agent such as temozolomide or an analog or derivative thereof that ranges from, for example, 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). In particular embodiments, a therapeutically effective amount of an alkylating agent includes 0.001 to 1,000 mg/kg (e.g., 1-5, 1-10, 1-20, 1-50, 1-100, 1-250, 1-500, 1-1,000, 10-50, 10-100, 10-250, 10-500, 10-1,000, 100-250, 100-500, or 100-1,000 mg/kg). In particular embodiments, a therapeutically effective amount of a selection agent can be administered subcutaneously or intravenously. In particular embodiments, a therapeutically effective amount of selection agent can be administered before, at the same time as, or after administration of one or more immunosuppression agents or immunosuppression regimens, one or more mobilization factors, one or more vectors, and/or one or more nucleic acids of the present disclosure.

In various embodiments of the present disclosure, an in vivo, in vitro, and or ex vivo gene therapy includes administration of at least one viral vector to a subject in combination with at least one selection agent. In an in vivo, in vitro, and or ex vivo gene therapy including more than one selection agent, the first selection agent and the second selection agent can be administered in a single formulation or dosage form or in two separate formulations or dosage forms. In various embodiments, the first and second selection agent can be administered at the same time or at different times, e.g., during the same one-hour period or during non-overlapping one-hour periods. In various embodiments, the first and second selection agent can be administered at the same time or at different times, e.g., on the same day or on different days.

In various embodiments, a selection agent is administered to a subject in a single total dose on a single day. In various embodiments, a selection agent is administered in two, three, four, or more unit doses that together constitute a total dose. In various embodiments, one unit dose of a selection agent is administered to a subject per day on each of one, two, three, four, or more consecutive days. In various embodiments, two unit doses of a selection agent are administered to a subject per day on each of one, two, three, four, or more consecutive days. Accordingly, in various embodiments, a daily dose can refer to the dose of selection agent received by a subject over the course of a day. In various embodiments, the term day refers to a twenty-four-hour period, such as a twenty-four-hour period from midnight of a first calendar date to midnight of the next calendar date.

### Kits

The present disclosure provides kits that include an editing nucleic acid of the present disclosure. In certain embodiments, the present disclosure provides kits that include a pharmaceutical composition disclosed herein and at least one additional composition for use in a method of gene therapy. In some embodiments, a kit of the present disclosure can include an editing nucleic acid and one or more MGMT inhibitors (e.g., O⁶BG or an analog or derivative thereof). In some embodiments, a kit of the present disclosure can include an editing nucleic acid and one or more alkylating agents (e.g., BCNU and/or temozolomide). In some embodiments, a kit of the present disclosure can include an editing nucleic acid and a selection regimen. In some embodiments, a kit of the present disclosure can include an editing nucleic acid and one or more hematopoietic cell mobilization agents. In some embodiments, a kit of the present disclosure can include an editing nucleic acid and one or more immunosuppression agents. In various embodiments, a kit can include instructions for selecting for modified cells. In some embodiments, a kit of the present disclosure can include an editing nucleic acid (optionally present in a pharmaceutically acceptable formulation) and a plurality of additional agents including, without limitation, one or more of an MGMT inhibitor, an alkylating agent, a selection regimen, a mobilization agent, an immunosuppression agent, and/or instructions for use thereof, e.g., in a method of gene therapy and/or a method of selecting for modified cells.

### EXAMPLES

The present Examples provide mutations that, when present in an MGMT polypeptide, unexpectedly confer inhibitor resistance to the MGMT polypeptide. The present Examples additionally provide assays useful in characterizing inhibitor-resistant MGMT polypeptides. The present Examples illustrate that editing systems can be engineered to target an MGMT-encoding nucleic acid in order to produce a modified MGMT-encoding nucleic acid that encodes an inhibitor-resistant MGMT. The present Examples further include that such editing systems can be delivered to a subject (e.g., using a viral vector) to modify cells of a subject. Moreover, administration of an editing system exemplified herein to a subject permits selection of modified cells (and selective elimination of non-modified cells) by administration of a selection regimen. Nucleic acids encoding editing systems exemplified herein can further deliver a therapeutic payload (e.g., a multiplexed editing system that includes a therapeutic component) to cells, whereby cells receiving the therapeutic payload that treats a target disease, disorder, or condition can be selected for. Such methods can increase the prevalence of cells that received the therapeutic payload to improve treatment of the disease, disorder, or condition.

### Example 1: Assays of MGMT Activity

The present Example provides methods that can be used to assess the activity of inhibitor-resistant MGMT (including, e.g., inhibitor-resistant MGMT encoded by modified MGMT-encoding nucleic acids). The present Example contemplates at least the following samples and conditions for analysis: purified samples of inhibitor-resistant cells or MGMT polypeptides (in the presence or absence of an MGMT inhibitor), purified samples of reference cells or polypeptides (in the presence or absence of an MGMT inhibitor), and samples including both inhibitor-resistant cells or MGMT polypeptides and reference cells or MGMT polypeptides (in the presence or absence of an MGMT inhibitor). Samples can be, without limitation, isolated cells or isolated polypeptides. Examples of samples including both reference MGMT polypeptides and inhibitor-resistant MGMT polypeptides can include samples isolated from, or in vivo assessment in, a subject or system to which an MGMT editing system was delivered.

Various assays can include assessment of any of one or more of i) expression and stability within mammalian cells, ii) endogenous alkyltransferase activity level, iii) resistance to inhibition by O⁶-BG (or other O⁶-benzylguanine analogues such as PaTrin-2), iv) ability to confer a proliferative advantage to HSCs following exposure to both an alkylating agent and O⁶-benzylguanine based inhibitor. A secondary criterion to assess can include the presence of a selective disadvantage conferred by the inhibitor-resistant MGMT in the absence of external selection pressures (e.g. alkylating agents and O⁶-BG).

The present Example includes, in part, an in vitro assay of [³H] methyl group transfer to MGMT under protein-limiting conditions. Highly specific radioactivity [3H]-methylated DNA substrate is incubated with cellular extract containing MGMT under protein-limiting conditions until the transfer reaction is complete or until a fixed time point. Excess substrate DNA is hydrolyzed to acid solubility and radioactivity in the residual protein is measured by liquid scintillation counting. Further description of certain such assays is found in Watson ((2000). O⁶-Alkylguanine-DNA Alkyltransferase Assay. In DNA Repair Protocols (pp. 49-61). Springer), which is incorporated herein by reference.

The present Example includes, in part, an in vitro competition assay that directly compares differences in proliferative advantage during or following exposure to alkylating agent and O⁶-BG. Cells (e.g., K562 or A549 cells) are transduced with plasmids or viral vectors (e.g. lentiviral vectors or integrating HDAd vectors) encoding, e.g., an MGMT editing system or an MGMT polypeptide. Cells are challenged with alkylating agent (e.g., 10 μM temozolomide) and O⁶-BG (e.g., 10 μM). Survival can be assessed, e.g., 3 and 6 days later via FACS. Further description of certain such assays is found in Woolford (2006 J. Gene Med. 8 (1): 29-34), which is incorporated herein by reference.

The present Example includes, in part, an in vitro assay of MGMT activity in which the MGMT is expressed from a transgene introduced into a cell genome by a viral vector. In some instances, an assay can utilize a two-vector HDAd system in which one vector (HDAd-GFP/MGMT) encodes MGMT and GFP within an integrating transposon flanked by inverted repeat (IR) and Flp Recognition Target (FRT) sites and the other vector (HDAd-SB) encodes Sleeping Beauty transposase and flp recombinase for circularization and integration of the MGMT-containing transposon. HUDEP-2 cells are transduced with both vectors. Erythroid differentiation of cells is initiated and cells are treated with multiple rounds of O⁶-BG (e.g., 50 μM) and BCNU (e.g., 10 μM and then 25 μM), e.g., at days 18 and 25. Positive selection of transduced cells can be assayed by flow cytometry. In some instances, a plasmid could be used to transfect HUDEP-2 cells in place of transduction with the HDAd-GFP/MGMT vector, with plasmid transfection one day prior to HDAd-SB transduction.

The present Example includes, in part, an in vitro assay that includes a fluorogenic probe of MGMT activity. A fluorogenic probe to assay for MGMT activity offers a straightforward, high dynamic range system that does not rely on radioactive reagents. The chemosensors operate via incorporation of a fluorophore and quencher pair, which become separated by the MGMT dealkylation reaction, yielding light-up responses of up to 55-fold, directly reflecting repair activity. Further description of certain such assays is found in Beharry (2016 PLoS ONE 11 (4): 1-15), which is incorporated herein by reference.

The present Example includes, in part, an in vitro fluorescence multiplex host cell reactivation (FM-HCR) assay in which a reporter plasmid with O⁶MeG-containing oligonucleotides is used. The oligonucleotides induce transcription errors that lead to expression of fluorescent mPlum protein. Functional MGMT will repair the O⁶MeG sites and reduce mPlum expression. This represents a rapid and validated assay system to assess intracellular activity of MGMT. Further description of certain such assays is found in Nagel (2014 PNAS USA 111 (18): E1823-32, which is incorporated herein by reference.

The present Example includes, in part, an in vitro survival assay using bacteria. Bacteria (e.g., E. coli) are engineered to express an MGMT polypeptide (e.g., an inhibitor-resistant MGMT polypeptide or a reference MGMT polypeptide). Bacteria are then contacted with a selection regimen including an alkylating agent (e.g., methylnitronitrosoguanidine (MNNG)) and cell survival of cells is subsequently assayed, e.g., as compared to a reference (e.g., reference cells and/or cells not contacted with the selection regimen). Further description of certain such assays is found in Pegg (1998 Cancer Res. 59:1936-1945) and Xu-Welliver (1999 Cancer Res. 59 (7): 1514-1519), which are incorporated herein by reference.

The present Example includes, in part, an in vivo assay in mice. Mice (e.g., CD46tg mice) are mobilized with G-CSF and AMD3100 and treated with dexamethasone. A two-vector HDAd system including HDAd-SB and HDAd-GFP/MGMT vectors is administered intravenously. Mice are subsequently administered O⁶BG/BCNU (e.g., in four doses at week 4, 6, 8, and 10 after HDAd administration). The O⁶BG dose can be, e.g., 30 mg/kg. BCNU doses can be, e.g., 5, 7.5, 10, 10, mg/kg corresponding in order to the four doses. Biweekly beginning at week 4, the fraction of gene modified PBMCs or RBCs can be assessed via qPCR or flow cytometry. The fraction of gene modified HSCs can be assessed from bone marrow samples. Further description of certain such assays is found in Wang (2020 JCI Insight 5 (16), 1-17), which is incorporated herein by reference.

### Example 2: Characterization of O(6)-Methylguanine Binding and Inhibitor Binding

The present Example provides an analysis of the binding site of MGMT with O(6)-methylguanine and the MGMT inhibitor O⁶BG. A model of binding between MGMT and each of O(6)-methylguanine and O⁶BG was prepared based on structures of wild-type MGMT protein alone, wild-type MGMT protein covalently bound to alkylguanine, and a modified MGMT^{C145S} bound to O⁶-benzylguanine (O⁶BG). Certain amino acid positions in which mutations of the present disclosure are found (Table 1) including positions 33, 134, 135, 137, 138, 140, 156, 158, 159, and 160 were in close proximity to the phenyl group in O⁶BG. MGMT mutations listed in Table 1, which mutations unexpectedly produce inhibitor-resistant MGMT polypeptides, were found to disrupt interaction of MGMT with the MGMT inhibitor O⁶BG while retaining some or all ability bind O(6)-methylguanine.

### Example 3. Characterization of MGMT Variants that are MGMT Inhibitor Resistant

The present Example demonstrates that exemplary MGMT variants disclosed in Table 1 and Table 2 are resistant to the MGMT inhibitor O⁶BG. The present Example uses an in vitro FM-HCR assay in which a reporter plasmid with 0° MeG-containing oligonucleotides is used to assay MGMT activity in cells. The O⁶MeG-containing oligonucleotides induce transcription errors that lead to expression of fluorescent mPlum protein. Functional MGMT will repair the O'MeG sites and reduce mPlum expression. This represents a rapid and validated assay system to assess intracellular activity of MGMT. Further description of certain such assays is found in Nagel et al., 2014 PNAS USA 111 (18): E1823-32 and Piett et al., 2021 Nat. Protoc. 16 (9): 4265-4298, each of which is incorporated herein by reference.

The in vitro FM-HCR assay was performed using the U251 human glioblastoma cell line. The cells were cultured in DMEM with 10% FBS and passaged every three days.

To express MGMT variants in the cells, plasmids encoding each MGMT variant were designed and generated by either de novo synthesis or cloning into the pTwist EF1 Alpha Puro vector at the HindII-BamHI multiple cloning site. The pTwist EF1 Alpha Puro plasmid vector drives expression of the MGMT transcript from a human EF1-alpha promoter including intron 1 and an SV40 polyadenylation signal. Plasmids encoding the MGMT variants were purified using standard molecular biology techniques from E. coli with low endotoxins. Plasmids encoding wild-type MGMT or O⁶BG-resistant MGMT^{P140K} were also designed and generated for use as controls.

To perform the in vitro FM-HCR assay, U251 cells were seeded in 12-well plates at a density of 50,000 cells per well in 1 mL culture media. The following day, the media was changed to 1 mL of culture media containing O⁶BG (100 μM O⁶BG and 0.1% DMSO final) or 0.1% DMSO; and the cells were transfected with 1,000 ng of a plasmid cocktail using Lipofectamine 3000 (ThermoFisher Scientific) at 1.8 μL Lipofectamine 3000 Reagent and 2 μL P3000 reagent in Opti-MEM medium (ThermoFisher Scientific). For the “damaged” condition, the plasmid cocktail comprised (i) 250 ng of an MGMT encoding plasmid, (ii) 100 ng of mPlum 0° MeG reporter plasmid, (iii) 100 ng of plasmid encoding blue fluorescent protein (BFP), and (vi) 550 ng of carrier plasmid that does not encode any fluorescent protein. For the “undamaged” condition, the plasmid cocktail replaced the mPlum O⁶MeG reporter plasmid with 100 ng of a plasmid expressing mPlum. The plasmid encoding BFP was used as an internal transfection control. For transfections where no MGMT encoding plasmid is used (no pMGMT), 250 ng of additional carrier plasmid was used to maintain a total DNA amount of 1,000 ng in each transfection. Four hours after transfection, the transfection medium was changed.

All transfected cells were analyzed by flow cytometry at 24-hours after transfection. Data was analyzed as previously described in Nagel et al., 2014 PNAS USA 111 (18): E1823-32 and Piett et al., 2021 Nat. Protoc. 16 (9): 4265-4298. For each condition, normalized reporter expression was determined by taking the product of the mPlum-positive cell count and the mean mPlum fluorescence intensity in mPlum-positive cells and dividing it by the product of the BFP-positive cell count and the mean BFP fluorescence intensity in BFP-positive cells. Next, for each MGMT variant, the percent reporter expression was determined by taking the normalized reporter expression in the “damaged” condition and dividing it by the normalized reporter expression in the “undamaged” condition, and then multiplying by 100 to obtain a percent value.

Table 25 shows in vitro FM-HCR assay results for the tested MGMT variants. For each MGMT variant, the mean percent reporter expression for the DMSO condition and the O⁶BG condition is shown, along with their associated standard deviations and the number of replicates (n). Additionally, Table 25 indicates for each MGMT variant whether prime editing and/or base editing can be used to modify an endogenous MGMT-encoding nucleic acid to produce a modified nucleic acid that encodes the MGMT variant.

TABLE 25
FM-HCR Assay Results

| | | | DMSO | | | O⁶BG | | |
|---|---|---|---|---|---|---|---|---|
| | | | Reporter | | | Reporter | | |
| MGMT | | | expression (%) | | | expression (%) | | |
| variant | Prime | Base | mean | stdev | n | mean | stdev | n |
| no pMGMT | — | — | 100.0 | 0.0 | 4 | 100.0 | 0.0 | 3 |
| WT | — | — | 18.5 | 2.1 | 4 | 70.0 | 6.6 | 4 |
| P140K | yes | — | 30.2 | 5.7 | 5 | 34.8 | 2.9 | 5 |
| L33F | yes | yes | 14.3 | 0.9 | 3 | 77.0 | 9.3 | 3 |
| L33K | yes | — | 16.0 | 2.1 | 4 | 62.3 | 1.5 | 4 |
| L33P | yes | yes | 12.3 | 0.5 | 3 | 54.7 | 1.2 | 3 |
| L33R | yes | — | 15.0 | 0.8 | 3 | 58.3 | 3.1 | 3 |
| L33W | yes | — | 14.3 | 0.5 | 3 | 57.7 | 8.0 | 3 |
| L33Y | yes | — | 17.0 | 0.7 | 4 | 77.3 | 13.4 | 4 |
| M134F | yes | — | 51.3 | 4.9 | 3 | 97.0 | 11.4 | 3 |
| M134V | yes | yes | 17.8 | 2.3 | 4 | 66.0 | 5.8 | 4 |
| M134W | yes | — | 103.5 | 11.8 | 4 | 112.3 | 11.4 | 3 |
| M134Y | yes | — | 108.0 | 6.2 | 3 | 106.3 | 13.1 | 3 |
| R135G | yes | yes | 20.0 | 0.8 | 3 | 106.0 | 11.3 | 3 |
| R135K | yes | yes | 13.3 | 0.5 | 3 | 84.3 | 13.3 | 3 |
| R135L | yes | — | 42.8 | 4.3 | 4 | 82.5 | 9.2 | 4 |
| R135T | yes | yes | 27.3 | 1.9 | 3 | 101.7 | 15.2 | 3 |
| N137D | yes | yes | 52.3 | 3.4 | 3 | 80.7 | 9.7 | 3 |
| N137F | yes | — | 109.7 | 7.4 | 3 | 104.3 | 11.1 | 3 |
| N137P | yes | — | 94.0 | 9.4 | 3 | 93.0 | 10.6 | 3 |
| P138K | yes | — | 16.3 | 1.2 | 3 | 64.0 | 8.3 | 3 |
| P140F | yes | yes | 25.3 | 10.8 | 4 | 76.8 | 31.6 | 4 |
| P140H | yes | — | 15.7 | 1.2 | 3 | 29.3 | 2.6 | 3 |
| P140I | yes | — | 14.7 | 1.2 | 3 | 31.0 | 5.0 | 3 |
| P140Q | yes | — | 15.3 | 0.5 | 3 | 49.0 | 4.2 | 3 |
| P140R | yes | yes | 25.3 | 2.1 | 3 | 38.3 | 2.1 | 3 |
| G156A | yes | yes | 32.3 | 5.4 | 4 | 79.0 | 28.5 | 4 |
| G156I | yes | — | 42.0 | 1.4 | 3 | 54.0 | 3.3 | 3 |
| G156P | yes | yes | 41.0 | 1.6 | 3 | 47.3 | 2.9 | 3 |
| G156V | yes | — | 36.8 | 3.6 | 4 | 55.0 | 4.5 | 4 |
| Y158F | yes | — | 12.3 | 1.2 | 3 | 61.3 | 3.3 | 3 |
| Y158H | yes | yes | 23.3 | 1.2 | 3 | 30.3 | 4.8 | 3 |
| Y158M | yes | — | 47.7 | 2.6 | 3 | 60.0 | 9.2 | 3 |
| Y158W | yes | — | 26.0 | 2.2 | 3 | 86.7 | 3.9 | 3 |
| S159F | yes | yes | 23.0 | 1.6 | 3 | 102.7 | 5.0 | 3 |
| S159I | yes | — | 18.0 | 2.4 | 3 | 40.3 | 6.8 | 3 |
| S159W | yes | yes | 31.3 | 2.6 | 3 | 108.0 | 7.9 | 3 |
| S159Y | yes | — | 26.0 | 5.4 | 3 | 142.7 | 11.0 | 3 |
| G160A | yes | yes | 15.5 | 1.7 | 4 | 74.5 | 1.7 | 4 |
| G160D | yes | — | 23.0 | 2.8 | 4 | 72.8 | 3.3 | 4 |
| G160E | yes | yes | 15.3 | 0.8 | 4 | 63.3 | 4.1 | 4 |
| G160H | yes | — | 10.3 | 1.2 | 3 | 21.3 | 2.1 | 3 |
| G160P | yes | yes | 22.8 | 5.1 | 4 | 47.5 | 9.7 | 4 |
| A170S | yes | — | 15.8 | 2.9 | 4 | 85.0 | 9.2 | 4 |

Table 26 shows analysis of the in vitro FM-HCR assay results presented in Table 25. As percent reporter expression shown in Table 25 is inversely related to MGMT activity, MGMT activity in DMSO and O⁶BG conditions can be determined and is presented in Table 26 as arbitrary units (a.u.), and also as a percentage relative to wild-type MGMT activity in the same condition. Additionally, Table 26 shows, for each MGMT variant, the ratio of the percent reporter expression for O⁶BG relative to DMSO. Lower values for this ratio are indicative of resistance to O⁶BG, while higher values are indicative of sensitivity to O⁶BG. Similarly, for each MGMT variant, the relative MGMT activity in the O⁶BG condition and the DMSO condition was calculated by dividing the respective MGMT activity (a.u.) values and then multiplying by 100 to obtain a percent value. Higher values for relative MGMT activity are indicative of resistance to O⁶BG, while lower values are indicative of sensitivity to O⁶BG. Finally, for each MGMT variant, O⁶BG resistance relative to wild-type MGMT was calculated as the percentage difference in relative MGMT activity (%) between the MGMT variant and wild-type MGMT. Positive values for O⁶BG resistance relative to WT are indicative of MGMT variants that have increased resistance to O⁶BG, while negative values are indicative of MGMT variants have increased sensitivity to O⁶BG.

TABLE 26
FM-HCR Assay Analysis

| | | | | | Ratio | | |
|---|---|---|---|---|---|---|---|
| | | | | | percent | | |
| | | | | | reporter | Relative | O⁶BG |
| | MGMT activity | | MGMT activity | | expression | MGMT | resistance |
| MGMT | (a.u.) | | (% of WT) | | (O⁶BG/ | activity | relative to |
| variant | DMSO | O⁶BG | DMSO | O⁶BG | DMSO) | (%) | WT (%) |
| no pMGMT | — | — | — | — | 1.00 | — | — |
| WT | 5.41 | 1.43 | 100.0 | 100.0 | 3.78 | 26.4 | 0.0 |
| P140K | 3.31 | 2.87 | 61.3 | 201.1 | 1.15 | 86.8 | 228.4 |
| L33F | 6.98 | 1.30 | 129.1 | 90.9 | 5.37 | 18.6 | −29.6 |
| L33K | 6.25 | 1.61 | 115.6 | 112.4 | 3.89 | 25.7 | −2.7 |
| L33P | 8.11 | 1.83 | 150.0 | 128.0 | 4.43 | 22.6 | −14.6 |
| L33R | 6.67 | 1.71 | 123.3 | 120.0 | 3.89 | 25.7 | −2.7 |
| L33W | 6.98 | 1.73 | 129.1 | 121.4 | 4.02 | 24.9 | −6.0 |
| L33Y | 5.88 | 1.29 | 108.8 | 90.6 | 4.54 | 22.0 | −16.7 |
| M134F | 1.95 | 1.03 | 36.0 | 72.2 | 1.89 | 52.9 | 100.2 |
| M134V | 5.63 | 1.52 | 104.2 | 106.1 | 3.72 | 26.9 | 1.8 |
| M134W | 0.97 | 0.89 | 17.9 | 62.3 | 1.09 | 92.1 | 248.6 |
| M134Y | 0.93 | 0.94 | 17.1 | 65.8 | 0.98 | 101.6 | 284.3 |
| R135G | 5.00 | 0.94 | 92.5 | 66.0 | 5.30 | 18.9 | −28.6 |
| R135K | 7.50 | 1.19 | 138.8 | 83.0 | 6.33 | 15.8 | −40.2 |
| R135L | 2.34 | 1.21 | 43.3 | 84.8 | 1.93 | 51.8 | 96.1 |
| R135T | 3.66 | 0.98 | 67.7 | 68.9 | 3.72 | 26.9 | 1.7 |
| N137D | 1.91 | 1.24 | 35.4 | 86.8 | 1.54 | 64.9 | 145.5 |
| N137F | 0.91 | 0.96 | 16.9 | 67.1 | 0.95 | 105.1 | 297.7 |
| N137P | 1.06 | 1.08 | 19.7 | 75.3 | 0.99 | 101.1 | 282.4 |
| P138K | 6.12 | 1.56 | 113.3 | 109.4 | 3.92 | 25.5 | −3.4 |
| P140F | 3.96 | 1.30 | 73.3 | 91.2 | 3.04 | 32.9 | 24.5 |
| P140H | 6.38 | 3.41 | 118.1 | 238.6 | 1.87 | 53.4 | 102.1 |
| P140I | 6.82 | 3.23 | 126.1 | 225.8 | 2.11 | 47.3 | 79.0 |
| P140Q | 6.52 | 2.04 | 120.7 | 142.9 | 3.20 | 31.3 | 18.4 |
| P140R | 3.95 | 2.61 | 73.0 | 182.6 | 1.51 | 66.1 | 150.1 |
| G156A | 3.10 | 1.27 | 57.4 | 88.6 | 2.45 | 40.8 | 54.5 |
| G156I | 2.38 | 1.85 | 44.0 | 129.6 | 1.29 | 77.8 | 194.3 |
| G156P | 2.44 | 2.11 | 45.1 | 147.9 | 1.15 | 86.6 | 227.8 |
| G156V | 2.72 | 1.82 | 50.3 | 127.3 | 1.50 | 66.8 | 152.8 |
| Y158F | 8.11 | 1.63 | 150.0 | 114.1 | 4.97 | 20.1 | −23.9 |
| Y158H | 4.29 | 3.30 | 79.3 | 230.8 | 1.30 | 76.9 | 191.1 |
| Y158M | 2.10 | 1.67 | 38.8 | 116.7 | 1.26 | 79.4 | 200.6 |
| Y158W | 3.85 | 1.15 | 71.2 | 80.8 | 3.33 | 30.0 | 13.5 |
| S159F | 4.35 | 0.97 | 80.4 | 68.2 | 4.46 | 22.4 | −15.2 |
| S159I | 5.56 | 2.48 | 102.8 | 173.6 | 2.24 | 44.6 | 68.9 |
| S159W | 3.19 | 0.93 | 59.0 | 64.8 | 3.45 | 29.0 | 9.8 |
| S159Y | 3.85 | 0.70 | 71.2 | 49.1 | 5.49 | 18.2 | −31.0 |
| G160A | 6.45 | 1.34 | 119.4 | 94.0 | 4.81 | 20.8 | −21.3 |
| G160D | 4.35 | 1.37 | 80.4 | 96.2 | 3.16 | 31.6 | 19.6 |
| G160E | 6.56 | 1.58 | 121.3 | 110.7 | 4.15 | 24.1 | −8.8 |
| G160H | 9.68 | 4.69 | 179.0 | 328.1 | 2.06 | 48.4 | 83.3 |
| G160P | 4.40 | 2.11 | 81.3 | 147.4 | 2.09 | 47.9 | 81.2 |
| A170S | 6.35 | 1.18 | 117.5 | 82.4 | 5.40 | 18.5 | −29.9 |

### Example 4: Prime Editing Systems that Introduce a P140K (AAA) MGMT Modification

Amino acid position 140 of a reference endogenous MGMT gene is a proline encoded by a CCC codon. Modification of the endogenous MGMT gene to instead encode a lysine at position 140 (e.g., by editing of the codon to AAA) causes the endogenous gene to encode an inhibitor-resistant MGMT polypeptide. The modified MGMT polypeptide of the present Example can be referred to as MGMT^{P140K}. The present Example includes prime editing systems that can modify an endogenous MGMT gene to produce a modified MGMT gene that encodes an inhibitor-resistant MGMT^{P140K} polypeptide.

pegRNAs can be generated by introducing user-designed target-specific nucleic acids into a pegRNA acceptor plasmid. The present Example utilizes the publicly available acceptor plasmid pU6-pegRNA-GG-acceptor (Addgene plasmid #132777). The pegRNA acceptor plasmid encodes and can express a pegRNA following introduction into the plasmid of a spacer and a 3′ extension (including the PBS and RT template).

The present Example includes pegRNAs characterized by a spacer selected from Table 27, an RT template selected from Table 28, and a PBS sequence selected from Table 29 in the following tables. Information presented in Table 27 additionally includes the strand orientation of the spacer sequence (whether a portion of a sequence such as SEQ ID NO: 3, 4, 5, 6, or 7, or NG_052673, that corresponds to the spacer sequence is present in a “sense” or “antisense” strand, e.g., relative to a provided sequence or a sequence encoding all or a portion of an MGMT polypeptide), the distance between the spacer sequence and the nearest edited nucleotide of the target sequence, and whether the PAM site is disrupted.

A secondary nicking sgRNA can also optionally be included to stimulate re-synthesis of the non-edited strand using the edited strand as a template, resulting in a fully edited duplex. sgRNAs can be generated by introducing user-designed target-specific nucleic acids into a sgRNA acceptor plasmid. The present Example utilizes the publicly available sgRNA acceptor plasmid, PE3. The sgRNA acceptor plasmid encodes and can express a sgRNA following introduction into the plasmid of a spacer sequence targeting the site to be nicked.

The present Example additionally includes secondary nicking sgRNAs characterized by a sequence selected from Table 30.

TABLE 27
Spacers

| | | Distance | Seed/ | SEQ |
|---|---|---|---|---|
| | | to Edit | PAM | ID |
| Sequence | Orientation | Start | Disrupt | NO: |
| TGTGGCACGGGATGAGGATG | antisense | 2 | 1 | 222 |
| CTGTGGCACGGGATGAGGAT | antisense | 3 | 1 | 223 |
| TCTGTGGCACGGGATGAGGA | antisense | 4 | 1 | 224 |
| TGGCTGCCCCCCTGTCTTCC | sense | 8 | 0 | 225 |
| CCACTCTGTGGCACGGGATG | antisense | 8 | 0 | 226 |
| TGCAGACCACTCTGTGGCAC | antisense | 14 | 0 | 227 |
| CTGCAGACCACTCTGTGGCA | antisense | 15 | 0 | 228 |
| CGCTGCTGCAGACCACTCTG | antisense | 20 | 0 | 229 |
| TCCAGATCCCTGACTGACAG | sense | 28 | 0 | 230 |
| TCCTCCGGAGTAGTTGCCCA | antisense | 46 | 0 | 231 |
| TTCCTTCACGGCCAGTCCTC | antisense | 61 | 0 | 232 |
| GGCCAGAAGCCATTCCTTCA | antisense | 73 | 0 | 233 |
| ACTCCTGTCAGTCAGGGCCT | sense | 74 | 0 | 234 |
| CACAGGACTCCTGTCAGTCA | sense | 80 | 0 | 235 |
| GCACAGGACTCCTGTCAGTC | sense | 81 | 0 | 236 |
| CCCCAACCGGTGGCCTTCAT | antisense | 94 | 0 | 237 |
| TCCCCAACCGGTGGCCTTCA | antisense | 95 | 0 | 238 |
| GGCCCCTGCTTGGTGGGCAC | sense | 97 | 0 | 239 |
| GGCACAGGCCCCTGCTTGGT | sense | 103 | 0 | 240 |
| AGCCTGGCTTCCCCAACCGG | antisense | 104 | 0 | 241 |
| TGGCACAGGCCCCTGCTTGG | sense | 104 | 0 | 242 |
| CCAAGCCTGGCTTCCCCAAC | antisense | 107 | 0 | 243 |
| CCCTGGCACAGGCCCCTGCT | sense | 107 | 0 | 244 |
| CAACAGCCTGCCCCTGGCAC | sense | 118 | 0 | 245 |
| CCTGAGCTCCCTCCCAAGCC | antisense | 120 | 0 | 246 |
| CCATGCCAACAGCCTGCCCC | sense | 124 | 0 | 247 |

TABLE 28
RT Templates

| | | SEQ ID |
|---|---|---|
| Sequence | Length | NO: |
| AGGTCAAAAT | 10 | 248 |
| CAGGTCAAAAT | 11 | 249 |
| CCAGGTCAAAAT | 12 | 250 |
| TCCAGGTCAAAAT | 13 | 251 |
| TTCCAGGTCAAAAT | 14 | 252 |
| CTTCCAGGTCAAAAT | 15 | 253 |
| TCTTCCAGGTCAAAAT | 16 | 254 |

TABLE 29
PBS Sequences

| | | SEQ ID |
|---|---|---|
| Sequence | Length | NO: |
| CCTCATCC | 8 | 255 |
| CCTCATCCC | 9 | 256 |
| CCTCATCCCG | 10 | 257 |
| CCTCATCCCGT | 11 | 258 |
| CCTCATCCCGTG | 12 | 259 |
| CCTCATCCCGTGC | 13 | 260 |
| CCTCATCCCGTGCC | 14 | 261 |
| CCTCATCCCGTGCCA | 15 | 262 |
| CCTCATCCCGTGCCAC | 16 | 263 |
| CCTCATCCCGTGCCACA | 17 | 264 |

TABLE 30
Secondary Nicking sgRNA Sequences

| | | | Distance | |
|---|---|---|---|---|
| | Nick | Orien- | to | SEQ |
| | Pos- | ta- | pegRNA | ID |
| Sequence | ition | tion | Nick | NO: |
| ACTCCTGTCAGTCAGGGCCT | 174 | Sense | 77 | 265 |
| CACAGGACTCCTGTCAGTCA | 168 | sense | 83 | 266 |
| GCACAGGACTCCTGTCAGTC | 167 | sense | 84 | 267 |
| GGCCCCTGCTTGGTGGGCAC | 151 | sense | 100 | 268 |
| GGCACAGGCCCCTGCTTGGT | 145 | sense | 106 | 269 |
| TGGCACAGGCCCCTGCTTGG | 144 | sense | 107 | 270 |
| CCCTGGCACAGGCCCCTGCT | 141 | sense | 110 | 271 |
| CAACAGCCTGCCCCTGGCAC | 130 | sense | 121 | 272 |
| CCATGCCAACAGCCTGCCCC | 124 | sense | 127 | 273 |
| GGTCTGCAGCAGCGGAGCCG | 291 | sense | −40 | 274 |
| GTCTGCAGCAGCGGAGCCGT | 292 | sense | −41 | 275 |
| GGAGCCGTGGGCAACTACTC | 304 | sense | −53 | 276 |
| GCCGTGGGCAACTACTCCGG | 307 | sense | −56 | 277 |
| GGGCAACTACTCCGGAGGAC | 312 | sense | −61 | 278 |
| CTCCGGAGGACTGGCCGTGA | 321 | sense | −70 | 279 |
| GAGGACTGGCCGTGAAGGAA | 326 | sense | −75 | 280 |
| GGCCGTGAAGGAATGGCTTC | 333 | sense | −82 | 281 |
| GAATGGCTTCTGGCCCATGA | 343 | sense | −92 | 282 |
| TTCTGGCCCATGAAGGCCAC | 350 | sense | −99 | 283 |
| GGCCCATGAAGGCCACCGGT | 354 | sense | −103 | 284 |
| GCCCATGAAGGCCACCGGTT | 355 | sense | −104 | 285 |
| CCCATGAAGGCCACCGGTTG | 356 | sense | −105 | 286 |
| GGCCACCGGTTGGGGAAGCC | 364 | sense | −113 | 287 |
| CCGGTTGGGGAAGCCAGGCT | 369 | sense | −118 | 288 |
| CGGTTGGGGAAGCCAGGCTT | 370 | sense | −119 | 289 |
| TTGGGGAAGCCAGGCTTGGG | 373 | sense | −122 | 290 |
| TGGGGAAGCCAGGCTTGGGA | 374 | sense | −123 | 291 |
| CCAGGCTTGGGAGGGAGCTC | 382 | sense | −131 | 292 |
| CTTGGGAGGGAGCTCAGGTC | 387 | sense | −136 | 293 |
| GGAGGGAGCTCAGGTCTGGC | 391 | sense | −140 | 294 |
| GAGGGAGCTCAGGTCTGGCA | 392 | sense | −141 | 295 |
| AGGGAGCTCAGGTCTGGCAG | 393 | sense | −142 | 296 |
| GCTCAGGTCTGGCAGGGGCC | 398 | sense | −147 | 297 |

### Example 5: Prime Editing Systems that Introduce a P140K(AAG) MGMT Modification

Due to the redundancy of the genetic code, a P140K inhibitor-resistant MGMT polypeptide can also be encoded by an endogenous MGMT gene modified to replace the CCC codon encoding P140 with a lysine-encoding AAG codon. The present Example includes prime editing systems that can modify an endogenous MGMT gene to produce a modified MGMT gene that encodes an inhibitor-resistant MGMT^{P140K} polypeptide. The present Example utilizes the pegRNA and sgRNA acceptor plasmids described in Example 4.

The present Example includes pegRNAs characterized by a spacer selected from Table 31, an RT template selected from Table 32, and a PBS sequence selected from Table 33 in the following tables. Information presented in Table 31 additionally includes the strand orientation of the spacer sequence, the distance between the spacer sequence and the nearest edited nucleotide of the target sequence, and whether the PAM site is disrupted.

The present Example additionally includes secondary nicking sgRNAs characterized by a sequence selected from Table 34.

TABLE 31
Spacers

| | Orien- | Distance | Seed/ | SEQ |
|---|---|---|---|---|
| | ta- | to Edit | PAM | ID |
| Sequence | tion | Start | Disrupt | NO: |
| TGTGGCACGGGATGAGGATG | Antisense | 2 | 1 | 298 |
| CTGTGGCACGGGATGAGGAT | Antisense | 3 | 1 | 299 |
| TCTGTGGCACGGGATGAGGA | Antisense | 4 | 1 | 300 |
| TGGCTGCCCCCCTGTCTTCC | sense | 8 | 0 | 301 |
| CCACTCTGTGGCACGGGATG | antisense | 8 | 0 | 302 |
| TGCAGACCACTCTGTGGCAC | antisense | 14 | 0 | 303 |
| CTGCAGACCACTCTGTGGCA | antisense | 15 | 0 | 304 |
| CGCTGCTGCAGACCACTCTG | antisense | 20 | 0 | 305 |
| TCCAGATCCCTGACTGACAG | sense | 28 | 0 | 306 |
| TCCTCCGGAGTAGTTGCCCA | antisense | 46 | 0 | 307 |
| TTCCTTCACGGCCAGTCCTC | antisense | 61 | 0 | 308 |
| GGCCAGAAGCCATTCCTTCA | antisense | 73 | 0 | 309 |
| ACTCCTGTCAGTCAGGGCCT | sense | 74 | 0 | 310 |
| CACAGGACTCCTGTCAGTCA | sense | 80 | 0 | 311 |
| GCACAGGACTCCTGTCAGTC | sense | 81 | 0 | 312 |
| CCCCAACCGGTGGCCTTCAT | antisense | 94 | 0 | 313 |
| TCCCCAACCGGTGGCCTTCA | antisense | 95 | 0 | 314 |
| GGCCCCTGCTTGGTGGGCAC | sense | 97 | 0 | 315 |
| GGCACAGGCCCCTGCTTGGT | sense | 103 | 0 | 316 |
| AGCCTGGCTTCCCCAACCGG | antisense | 104 | 0 | 317 |
| TGGCACAGGCCCCTGCTTGG | sense | 104 | 0 | 318 |
| CCAAGCCTGGCTTCCCCAAC | antisense | 107 | 0 | 319 |
| CCCTGGCACAGGCCCCTGCT | sense | 107 | 0 | 320 |
| CAACAGCCTGCCCCTGGCAC | sense | 118 | 0 | 321 |
| CCTGAGCTCCCTCCCAAGCC | antisense | 120 | 0 | 322 |
| CCATGCCAACAGCCTGCCCC | sense | 124 | 0 | 323 |

TABLE 32
RT Templates

| | | SEQ ID |
|---|---|---|
| Sequence | Length | NO: |
| AGGTCAAGAT | 10 | 324 |
| CAGGTCAAGAT | 11 | 325 |
| CCAGGTCAAGAT | 12 | 326 |
| TCCAGGTCAAGAT | 13 | 327 |
| TTCCAGGTCAAGAT | 14 | 328 |
| CTTCCAGGTCAAGAT | 15 | 329 |
| TCTTCCAGGTCAAGAT | 16 | 330 |

TABLE 33
PBS Sequences

| | | SEQ ID |
|---|---|---|
| Sequence | Length | NO: |
| CCTCATCC | 8 | 331 |
| CCTCATCCC | 9 | 332 |
| CCTCATCCCG | 10 | 333 |
| CCTCATCCCGT | 11 | 334 |
| CCTCATCCCGTG | 12 | 335 |
| CCTCATCCCGTGC | 13 | 336 |
| CCTCATCCCGTGCC | 14 | 337 |
| CCTCATCCCGTGCCA | 15 | 338 |
| CCTCATCCCGTGCCAC | 16 | 339 |
| CCTCATCCCGTGCCACA | 17 | 340 |

TABLE 34
Secondary Nicking sgRNA Sequences

| | | | Distance | |
|---|---|---|---|---|
| | Nick | Orien- | to | SEQ |
| | Pos- | ta- | pegRNA | ID |
| Sequence | ition | tion | Nick | NO: |
| ACTCCTGTCAGTCAGGGCCT | 174 | Sense | 77 | 341 |
| CACAGGACTCCTGTCAGTCA | 168 | Sense | 83 | 342 |
| GCACAGGACTCCTGTCAGTC | 167 | Sense | 84 | 343 |
| GGCCCCTGCTTGGTGGGCAC | 151 | Sense | 100 | 344 |
| GGCACAGGCCCCTGCTTGGT | 145 | Sense | 106 | 345 |
| TGGCACAGGCCCCTGCTTGG | 144 | Sense | 107 | 346 |
| CCCTGGCACAGGCCCCTGCT | 141 | Sense | 110 | 347 |
| CAACAGCCTGCCCCTGGCAC | 130 | Sense | 121 | 348 |
| CCATGCCAACAGCCTGCCCC | 124 | Sense | 127 | 349 |
| GGTCTGCAGCAGCGGAGCCG | 291 | Sense | −40 | 350 |
| GTCTGCAGCAGCGGAGCCGT | 292 | Sense | −41 | 351 |
| GGAGCCGTGGGCAACTACTC | 304 | Sense | −53 | 352 |
| GCCGTGGGCAACTACTCCGG | 307 | Sense | −56 | 353 |
| GGGCAACTACTCCGGAGGAC | 312 | Sense | −61 | 354 |
| CTCCGGAGGACTGGCCGTGA | 321 | Sense | −70 | 355 |
| GAGGACTGGCCGTGAAGGAA | 326 | Sense | −75 | 356 |
| GGCCGTGAAGGAATGGCTTC | 333 | Sense | −82 | 357 |
| GAATGGCTTCTGGCCCATGA | 343 | Sense | −92 | 358 |
| TTCTGGCCCATGAAGGCCAC | 350 | Sense | −99 | 359 |
| GGCCCATGAAGGCCACCGGT | 354 | Sense | −103 | 360 |
| GCCCATGAAGGCCACCGGTT | 355 | Sense | −104 | 361 |
| CCCATGAAGGCCACCGGTTG | 356 | Sense | −105 | 362 |
| GGCCACCGGTTGGGGAAGCC | 364 | Sense | −113 | 363 |
| CCGGTTGGGGAAGCCAGGCT | 369 | Sense | −118 | 364 |
| CGGTTGGGGAAGCCAGGCTT | 370 | Sense | −119 | 365 |
| TTGGGGAAGCCAGGCTTGGG | 373 | Sense | −122 | 366 |
| TGGGGAAGCCAGGCTTGGGA | 374 | Sense | −123 | 367 |
| CCAGGCTTGGGAGGGAGCTC | 382 | Sense | −131 | 368 |
| CTTGGGAGGGAGCTCAGGTC | 387 | Sense | −136 | 369 |
| GGAGGGAGCTCAGGTCTGGC | 391 | Sense | −140 | 370 |
| GAGGGAGCTCAGGTCTGGCA | 392 | Sense | −141 | 371 |
| AGGGAGCTCAGGTCTGGCAG | 393 | Sense | −142 | 372 |
| GCTCAGGTCTGGCAGGGGCC | 398 | Sense | −147 | 373 |

### Example 6: Prime Editing Systems that Introduce a G156A (GCG) MGMT Modification

Amino acid position 156 of a reference endogenous MGMT gene is a glycine encoded by a ggc codon. Modification of the endogenous MGMT gene to instead encode an alanine at position 156 (e.g., by editing of the codon to GCG) causes the endogenous gene to encode an inhibitor-resistant MGMT polypeptide. The modified MGMT polypeptide of the present Example can be referred to as MGMT^{G156A}

The present Example includes prime editing systems that can modify an endogenous MGMT gene to produce a modified MGMT gene that encodes MGMT^{G156A}. The present Example utilizes the pegRNA and sgRNA acceptor plasmids described in Example 4.

The present Example includes pegRNAs characterized by a spacer selected from Table 35, an RT template selected from Table 36, and a PBS sequence selected from Table 37 in the following tables. Information presented in Table 35 additionally includes the strand orientation of the spacer sequence, the distance between the spacer sequence and the nearest edited nucleotide of the target sequence, and whether the PAM site is disrupted.

The present Example additionally includes secondary nicking sgRNAs characterized by a sequence selected from Table 38.

TABLE 35
Spacers

| | Orien- | Distance | Seed/ | SEQ |
|---|---|---|---|---|
| | ta- | to Edit | PAM | ID |
| Sequence | tion | Start | Disrupt | NO: |
| GTCTGCAGCAGCGGAGCCGT | sense | 5 | 1 | 374 |
| GGTCTGCAGCAGCGGAGCCG | sense | 6 | 0 | 375 |
| TTCCTTCACGGCCAGTCCTC | antisense | 13 | 0 | 376 |
| CACAGAGTGGTCTGCAGCAG | sense | 14 | 0 | 377 |
| GGCCAGAAGCCATTCCTTCA | antisense | 25 | 0 | 378 |
| CCTCATCCCGTGCCACAGAG | sense | 27 | 0 | 379 |
| CCCCAACCGGTGGCCTTCAT | antisense | 46 | 0 | 380 |
| TCCCCAACCGGTGGCCTTCA | antisense | 47 | 0 | 381 |
| AGCCTGGCTTCCCCAACCGG | antisense | 56 | 0 | 382 |
| TGGCTGCCCCCCTGTCTTCC | sense | 57 | 0 | 383 |
| CCAAGCCTGGCTTCCCCAAC | antisense | 59 | 0 | 384 |
| CCTGAGCTCCCTCCCAAGCC | antisense | 72 | 0 | 385 |
| TCCAGATCCCTGACTGACAG | sense | 77 | 0 | 386 |
| AGCTCCCGCTCCCTTGAGCC | antisense | 106 | 0 | 387 |
| ACTCCTGTCAGTCAGGGCCT | sense | 123 | 0 | 388 |
| CACAGGACTCCTGTCAGTCA | sense | 129 | 0 | 389 |
| GCCAGCAGGCGGGGAGCCCG | antisense | 130 | 0 | 390 |
| GCACAGGACTCCTGTCAGTC | sense | 130 | 0 | 391 |
| TCAGTTTCGGCCAGCAGGCG | antisense | 139 | 0 | 392 |
| CTCAGTTTCGGCCAGCAGGC | antisense | 140 | 0 | 393 |
| ACTCAGTTTCGGCCAGCAGG | antisense | 141 | 0 | 394 |
| CATACTCAGTTTCGGCCAGC | antisense | 144 | 0 | 395 |
| GGCCCCTGCTTGGTGGGCAC | sense | 146 | 0 | 396 |

TABLE 36
RT Templates

| | | SEQ ID |
|---|---|---|
| Sequence | Length | NO: |
| GTTCGCCACG | 10 | 397 |
| AGTTCGCCACG | 11 | 398 |
| TAGTTCGCCACG | 12 | 399 |
| GTAGTTCGCCACG | 13 | 400 |
| AGTAGTTCGCCACG | 14 | 401 |
| GAGTAGTTCGCCACG | 15 | 402 |
| GGAGTAGTTCGCCACG | 16 | 403 |
| CGGAGTAGTTCGCCACG | 17 | 404 |

TABLE 37
PBS Sequences

| | | SEQ ID |
|---|---|---|
| Sequence | Length | NO: |
| GCTCCGCT | 8 | 405 |
| GCTCCGCTG | 9 | 406 |
| GCTCCGCTGC | 10 | 407 |
| GCTCCGCTGCT | 11 | 408 |
| GCTCCGCTGCTG | 12 | 409 |
| GCTCCGCTGCTGC | 13 | 410 |
| GCTCCGCTGCTGCA | 14 | 411 |
| GCTCCGCTGCTGCAG | 15 | 412 |
| GCTCCGCTGCTGCAGA | 16 | 413 |
| GCTCCGCTGCTGCAGAC | 17 | 414 |

TABLE 38
Secondary Nicking sgRNA Sequences

| | | | Distance | |
|---|---|---|---|---|
| | Nick | Orien- | to | SEQ |
| | Pos- | ta- | pegRNA | ID |
| Sequence | ition | tion | Nick | NO: |
| TCCTCCGGAGTAGTTCGCCA | 198 | antisense | 5 | 415 |
| CCCCAACCGGTGGCCTTCAT | 246 | antisense | 53 | 416 |
| TCCCCAACCGGTGGCCTTCA | 247 | antisense | 54 | 417 |
| AGCCTGGCTTCCCCAACCGG | 256 | antisense | 63 | 418 |
| CCAAGCCTGGCTTCCCCAAC | 259 | antisense | 66 | 419 |
| CCTGAGCTCCCTCCCAAGCC | 272 | antisense | 79 | 420 |
| AGCTCCCGCTCCCTTGAGCC | 306 | antisense | 113 | 421 |
| GCCAGCAGGCGGGGAGCCCG | 330 | antisense | 137 | 422 |
| ACGGGATGAGGATGGGGACC | 148 | antisense | −45 | 423 |
| TCAGTTTCGGCCAGCAGGCG | 339 | antisense | 146 | 424 |
| CTCAGTTTCGGCCAGCAGGC | 340 | antisense | 147 | 425 |
| ACTCAGTTTCGGCCAGCAGG | 341 | antisense | 148 | 426 |
| AGGATGGGGACCTGGAAGAC | 140 | antisense | −53 | 427 |
| GGATGGGGACCTGGAAGACA | 139 | antisense | −54 | 428 |
| GATGGGGACCTGGAAGACAG | 138 | antisense | −55 | 429 |
| ATGGGGACCTGGAAGACAGG | 137 | antisense | −56 | 430 |
| TGGGGACCTGGAAGACAGGG | 136 | antisense | −57 | 431 |
| GGGGGCAGCCACTGTCAGTC | 118 | antisense | −75 | 432 |
| GGGGCAGCCACTGTCAGTCA | 117 | antisense | −76 | 433 |
| GCCACTGTCAGTCAGGGATC | 111 | antisense | −82 | 434 |
| AGTCAGGGATCTGGACAACG | 102 | antisense | −91 | 435 |
| GATCTGGACAACGAGGTCTT | 95 | antisense | −98 | 436 |
| ATCTGGACAACGAGGTCTTT | 94 | antisense | −99 | 437 |
| TCTGGACAACGAGGTCTTTG | 93 | antisense | −100 | 438 |
| CAACGAGGTCTTTGGGGTCA | 87 | antisense | −106 | 439 |
| GGTCTTTGGGGTCAAGGCCA | 81 | antisense | −112 | 440 |
| AGGCCAAGGCCCTGACTGAC | 67 | antisense | −126 | 441 |
| GAGTCCTGTGCCCACCAAGC | 45 | antisense | −148 | 442 |
| AGTCCTGTGCCCACCAAGCA | 44 | antisense | −149 | 443 |
| GTCCTGTGCCCACCAAGCAG | 43 | antisense | −150 | 444 |

### Example 7: Multiplex Base Editing/Prime Editing for Enrichment of HBG-Edited HSCs

A significant obstacle in certain hematopoietic stem cell (HSC) gene therapy studies has been the inability to consistently and reliably achieve sufficiently high engraftment levels of gene-modified cells to provide long-term therapeutic efficacy. Subtherapeutic engraftment has been observed to occur even after fully myeloablative conditioning and is conceptually of still greater concern with nonmyeloablative and nongenotoxic conditioning. One remedy for subtherapeutic engraftment of gene-modified autologous cells is allogeneic (allo-) HSC transplantation, which is associated with significant toxicity from graft-versus-host disease and high-dose conditioning. This Example provides an alternative approach that enables increasing engraftment levels of gene-modified autologous cells into a therapeutic range after HSC gene therapy, thus avoiding the need for an allo-HSC transplant and associated toxicities. Post-engraftment selection of hereditary persistence of fetal hemoglobin (HPFH)-edited HSCs will raise HbF to therapeutic levels. To accomplish this, an engineered mutation in the O⁶-methylguanine DNA methyltransferase (MGMT) gene will be used. When virally delivered, the MGMT^{P140K}-based selection system significantly increases modified HSCs in large animals and patients (Adair et al., Sci Translat Med. 2012; 4 (133): 133ra57; Beard et al., J Clin Invest. 2010; 120 (7): 2345-54; Beard et al., Blood. 2009; 113 (21): 5094-103). To simultaneously generate HPFH alleles and a drug-resistance mutation for selection in the same cell, novel base editors (BE) as well as the newly described prime editor (PE) platform (Anzalone et al., Nature. 2019; 576 (7785): 149-57) will be used, which systems can be even safer than the more commonly used CRISPR/Cas9 platform at least in part because they introduce precise genetic alterations without double-stranded DNA breaks (DSBs), thus reducing potential for genotoxicity (Kosicki et al., Nat Biotechnol. 2018; 36 (8): 765-71).

CD34+ HSCs will be edited using PE and/or BE systems for targeting the therapeutic HBG site for HbF reactivation, and the selection gene MGMT for enrichment of therapeutically edited cells. Multilineage engraftment of edited HSCs will be assessed in vivo in a humanized NBSGW mouse xenotransplantation model (McIntosh et al., Stem Cell Reports. 2015; 4 (2): 171-80). In vitro and in vivo selection for HPFH-edited cells will be quantified following treatment with the respective drugs and by measuring editing efficiency before and after selection. Another group of mice will be transplanted with SCD cells to verify reversal of the SCD sickling phenotype at time of necropsy.

Representative Methods: Methods used may include those described in the following publications: Traxler et al., Nat Med. 2016; 22 (9): 987-90; Metais et al., Blood Adv. 2019; 3 (21): 3379-92; and Humbert et al., Leukemia. 2019; 33 (3): 762-808.

Human HSC's: De-identified, PB-mobilized CD34+ cells will be obtained from several sources, such as the Fred Hutch Cell Processing facility and commercial vendors. Plerixafor-mobilized or BM HSCs isolated from SCD patients will be obtained through several clinical research protocols opened at St. Jude. CD34+ cells will be enriched by immunomagnetic beads using the CliniMACS Plus system. The HSCs will be cultured in vitro as described (Metais et al., Blood Adv. 2019; 3 (21): 3379-92). Studies will be performed in CD34+ cells isolated from at least three different normal subjects and three individuals with SCD. Cells from each individual will be analyzed in at least two biological replicate experiments.

BE mRNA: mRNA encoding BE will be modified with CleanCap and 5′ N1 methyl pseudouridine to enhance mRNA stability and reduce innate cellular immune response (Uzri et al., J Virol. 2009; 83 (9): 4174-84; Vaidyanathan et al., Mol Ther Nucleic acids. 2018; 12:530-42). gRNAs will be sourced from Biosprings or Synthego, with 2′O-methyl analogs and 3′phosphorothioate internucleotide linkages at the first three 5′ and 3′ termini to protect from exonucleases and reduce innate immune response (Kim et al., Genome Res. 2018, doi: 10.1101/gr.231936.117.; Wienert et al., PLoS Biol. 2018; 16 (7): e2005840). The gRNAs and mRNAs will be purified by high performance liquid chromatography (HPLC) to isolate full-length products and eliminate DNA contamination and double-stranded RNA, which can induce innate immune responses.

Mouse xenotransplantation. The NBSGW mouse xenotransplantation model is widely used to evaluate multi-lineage engraftment of normal and genome-edited human HSCs (McIntosh et al., Stem Cell Reports. 2015; 4 (2): 171-80). Efficient engraftment of human CD34+ cells has been demonstrated in this model and differentiation into CD3+ (T-cells), CD34+ (HSCs), CD14+ (myeloid), CD19+ (B-cells), and CD235a (erythroid) cells that persisted for over 16 wks has been confirmed (Metais et al., Blood Adv. 2019; 3 (21): 3379-92). NBSGW mice support human donor cell erythropoiesis, with recipient BM containing up to 10% late-stage human erythroid precursors, mainly polychromatophilic erythroblasts and reticulocytes that can be purified and analyzed for HbF expression and in vitro sickling under hypoxia. For xenotransplantation studies, mice of 8-13 weeks of age are injected intravenously with 3-5×10⁵ edited or control CD34+ cells, without prior conditioning. Female mice will be used as xenotransplantation recipients, as this sex engrafts more efficiently with human donor cells (Notta et al., Blood. 2010; 115 (18): 3704-7). For engraftment studies using CD34+ HSCs from healthy patients, groups of 10 mice transplanted with single BE treated cells (HBG, MGMT, CD33) will be compared to multiplex BE treated cells (HBG+MGMT; HBG+CD33) or to unmanipulated cells. It is expected that genome-edited cells will engraft in a similar fashion to that seen in the mice that received unmanipulated cells based on results from CRISPR/Cas9 studies. After stable engraftment at 15-18 weeks post-transplant, 5 out of 10 mice will be treated with selection drugs to enrich for genome-edited cells. To account for donor to donor variability, cells from 3 different donors will be used for these purposes, resulting in 30 mice per treatment group (x 6 groups=180 mice). This number of biological replicates is sufficient to account for occasional mortality of transplanted mice and detect robust differences at statistical significance. For engraftment studies using CD34+ HSCs from SCD patients, only the multiplex approaches (HBG+MGMT; HBG+CD33) will be tested using a similar procedure as described above for healthy CD34+ cells, resulting in 60 mice (30×2 groups).

Selection for multiplex-edited cells. For MGMT-based selection in vitro, cells will be treated with 50 μM O⁶BG and 50 μM BCNU for 3 hr. Editing efficiency will be compared between treated and untreated cells in bulk populations and in clonal hematopoietic colony assays. Briefly, these assays are performed in Methocult H4230 methylcellulose media (STEMCELL Technologies, Vancouver, Canada) supplemented with 100 ng/ml each of recombinant human IL-3, IL-6, SCF, TPO, G-CSF, GM-CSF, and EPO. After stable engraftment at 15-18 weeks, mice will be treated with two doses of O⁶BG (15 mg/kg) administered intraperitoneally 30 min apart, followed by a dose of BCNU (5 mg/kg) to select for MGMT-edited cells. Selection drugs may be administered up to three times in each animal after hematopoietic recovery for further amplification of genome-edited cells.

Statistical analysis: The difference in engraftment of edited cells between each animal group will be assessed after stable engraftment (15-18 weeks) by comparing the means between two groups, totaling 15 mice per group, which will provide 91% power to observe a statistically significant difference (at the two-sided significance level of 0.05) in means if the true distributions are separated by 1.25 standard-deviation (sd) units (using a two-sample t-test). If one compares the means across 6 groups, if the distribution of one group is 1.25 sd units from the common distribution of each of the other 5 groups, 15 mice per group provides 93% power (using one-way ANOVA).

### Example 8: Base Editing of HBG

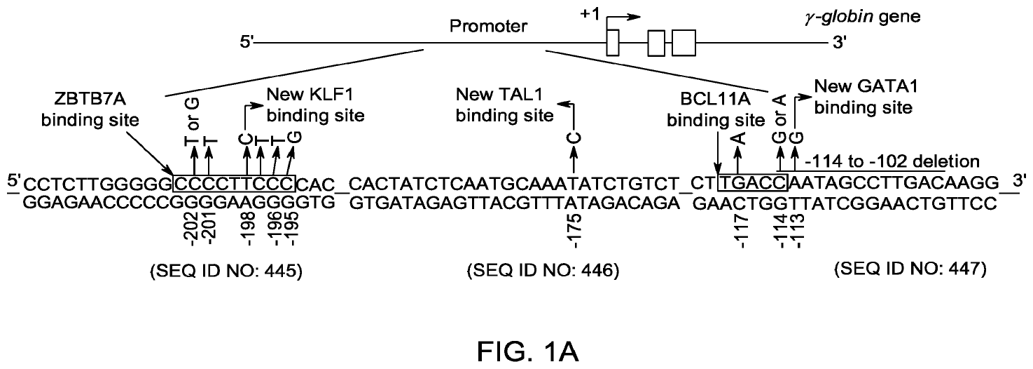

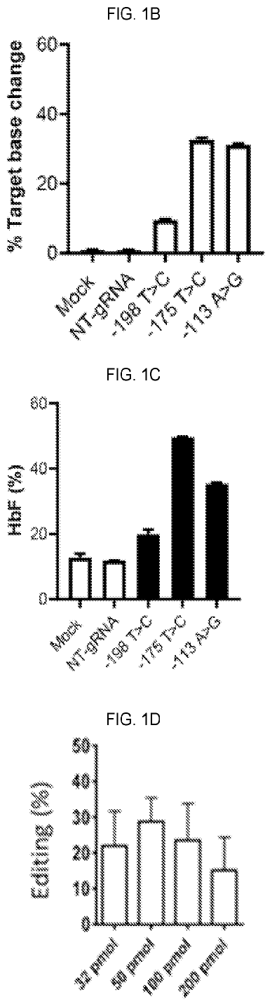

Most small (“non-deletional”) HPFH mutations alter the HBG gene promoter to either create new binding sites for an erythroid transcriptional activator, or disrupt a binding site for a transcriptional repressor, either BCL11A or ZBTB7A (Wienert et al., Trends Genet. 2018; 34 (12): 927-40). ABE 7.10 was used to generate three HPFH point mutations: −198 T>C, −175 T>C, and −113 A>G in human CD34+ HSCs (FIG. 1A). The cells were electroporated with RNPs consisting of ABE7.10 protein complexed with a targeting gRNA, then induced to undergo erythroid maturation. On-target editing was measured at day 3 by next generation sequencing (NGS) and showed up to 35% efficiency with a corresponding increase in protein HbF expression measured by HPLC in erythroid differentiated cells at day 15 (FIGS. 1B, IC). After further process optimization, on-target editing efficiency reached as much as 60% in human CD34+ HSCs. In additional experiments, the CBE BE4 (Komor et al., Sci Adv. 2017; 3 (8): eaao4774) was evaluated for editing of the same-113 HPFH site to inactivate the BCL11A binding site. Titration of CBE protein in human CD34+ HSC showed up to 30% editing efficiency (FIG. 1D) with significant HbF reactivation.

These data validate the use of both CBE and ABE for the targeting of HPFH sites and for increasing HbF expression. BE targeting the −175 and −113 sites yield the greatest HbF reactivation.

### REFERENCES

Anzalone, A. V., Randolph, P. B., Davis, J. R., Sousa, A. A., Koblan, L. W., Levy, J. M., Chen, P. J., Wilson, C., Newby, G. A., Raguram, A., & Liu, D. R. (2019). Search-and-replace genome editing without double-strand breaks or donor DNA. Nature, 576 (7785), 149-157.
Beharry, A. A., Nagel, Z. D., Samson, L. D., & Kool, E. T. K. (2016). Fluorogenic real-time reporters of DNA repair by MGMT, a clinical predictor of antitumor drug response. PLoS ONE, 11 (4), 1-15.
Chen, L., Park, J. E., Paa, P., Rajakumar, P. D., Chew, Y. T., Manivannan, S. N., & Chew, W. L. (2020). Precise and programmable C: G to G: C base editing in genomic DNA. BioRxiv.
Choi, J., Chen, W., Suiter, C. C., Lee, C., Chardon, F. M., Yang, W., Leith, A., Daza, R. M., Martin, B., & Shendure, J. (2021). Precise genomic deletions using paired prime editing. BioRxiv, 2012-2020.
Chow, R. D., Chen, J. S., Shen, J., & Chen, S. (2020). A web tool for the design of prime-editing guide RNAs. Nature Biomedical Engineering.
Christians, F. C., Dawson, B. J., Coates, M. M., & Loeb, L. A. (1997). Creation of human alkyltransferases resistant to O⁶-benzylguanine. Cancer Research, 57 (10), 2007-2012.
Cox, D. B. T., Gootenberg, J. S., Abudayyeh, O. O., Franklin, B., Kellner, M. J., Joung, J., & Zhang, F. (2017). RNA editing with CRISPR-Cas13. Science, 358 (6366), 1019-1027.
Crone, T. M., Goodtzova, K., Edara, S., & Pegg, A. E. (1994). Mutations inHuman Alkyltransferase Imparting Resistance. 6221-6227.
Daniels, D. S., Mol, C. D., Arvai, A. S., Kanugula, S., Pegg, A. E., & Tainer, J. A. (2000). Active and alkylated human AGT structures: A novel zinc site, inhibitor and extrahelical base binding. EMBO Journal, 19 (7), 1719-1730.
Daniels, D. S., Woo, T. T., Luu, K. X., Noll, D. M., Clarke, N. D., Pegg, A. E., & Tainer, J. A. (2004). DNA binding and nucleotide flipping by the human DNA repair protein AGT. Nature Structural and Molecular Biology, 11 (8), 714-720.
Davis, B. M., Encell, L. P., Zielske, S. P., Christians, F. C., Liu, L., Friebert, S. E., Loeb, L. A., & Gerson, S. L. (2001). Applied molecular evolution of O⁶-benzylguanine-resistant DNA alkyltransferases in human hematopoietic cells. Proceedings of the National Academy of Sciences of the United States of America, 98 (9): 4950-4954.
Edara, S., Kanugula, S., Goodtzova, K., & Pegg, A. E. (1996). Resistance of the human O⁶-alkylguanine-DNA alkyltransferase containing arginine at codon 160 to inactivation by O⁶-benzylguanine. Cancer Research, 56 (24), 5571-5575.
Encell, L. P., Coates, M. M., & Loeb, L. A. (1998). Engineering human DNA alkyltransferases for gene therapy using random sequence mutagenesis. Cancer Research, 58 (5), 1013-1020.
Fan, C. H., Liu, W. L., Cao, H., Wen, C., Chen, L., & Jiang, G. (2013). O⁶-methylguanine DNA methyltransferase as a promising target for the treatment of temozolomide-resistant gliomas. Cell Death and Disease, 4 (10).
Grünewald, J., Zhou, R., Lareau, C. A., Garcia, S. P., Iyer, S., Miller, B. R., Langner, L. M., Hsu, J. Y., Aryee, M. J., & Joung, J. K. (2020). A dual-deaminase CRISPR base editor enables concurrent adenine and cytosine editing. Nature Biotechnology, 38 (7), 861-864.
Hartner, J. C., Walkley, C. R., Lu, J., & Orkin, S. H. (2009). ADAR1 is essential for the maintenance of hematopoiesis and suppression of interferon signaling. Nature Immunology, 10 (1), 109-115.
Huang, T. P., Zhao, K. T., Miller, S. M., Gaudelli, N. M., Oakes, B. L., Fellmann, C., Savage, D. F., & Liu, D. R. (2019). Circularly permuted and PAM-modified Cas9 variants broaden the targeting scope of base editors. Nature Biotechnology, 37 (6), 626-631.
Kurt, I. C., Zhou, R., Iyer, S., Garcia, S. P., Miller, B. R., Langner, L. M., Grünewald, J., & Joung, J. K. (2020). CRISPR C-to-G base editors for inducing targeted DNA transversions in human cells. Nature Biotechnology, C.
Li, X., Wang, Y., Liu, Y., Yang, B., Wang, X., Wei, J., Lu, Z., Zhang, Y., Wu, J., Huang, X., Yang, L., & Chen, J. (2018). Base editing with a Cpf1-cytidine deaminase fusion. Nature Biotechnology, 36 (4), 324-327.
Liddicoat, B., Piskol, R., Chalk, A., Higuchi, M., Seeburg, P., Li, J. B., Hartner, J., & Walkley, C. R. (2013). A-To-I RNA editing by ADAR1 is essential for hematopoiesis. American Society of Hematology Washington, DC.
Montiel-Gonzalez, M. F., Vallecillo-Viejo, I., Yudowski, G. A., & Rosenthal, J. J. C. (2013). Correction of mutations within the cystic fibrosis transmembrane conductance regulator by site-directed RNA editing. Proceedings of the National Academy of Sciences of the United States of America, 110 (45), 18285-18290.
Montiel-Gonzalez, M. F., Vallecillo-Viejo, I. C., & Rosenthal, J. J. C. (2016). An efficient system for selectively altering genetic information within mRNAs. Nucleic Acids Research, 44 (21), 1-12.
Nagel, Z. D., Margulies, C. M., Chaim, I. A., McRee, S. K., Mazzucato, P., Ahmad, A., Abo, R. P., Butty, V. L., Forget, A. L., & Samson, L. D. (2014). Multiplexed DNA repair assays for multiple lesions and multiple doses via transcription inhibition and transcriptional mutagenesis. Proceedings of the National Academy of Sciences of the United States of America, 111 (18): E1823-32.
Pegg, A. E., Byers, T. L., Swenn, K., Boosalis, M., Samson, L., Moschel, R. C., & Dolan, M. E. (1993). Mechanism of Inactivation of Human O⁶-Alkylguanine-DNA Alkyltransferase by O⁶-Benzylguanine. Biochemistry, 32 (45), 11998-12006.
Pegg, A. E., Fang, Q., & Loktionova, N. A. (2007). Human variants of O⁶-alkylguanine-DNA alkyltransferase. DNA Repair, 6 (8), 1071-1078.
Margison, G. P., Povey, A. C., Kaina, B., & Santibáñez Koref, M. F. (2003). Variability and regulation of O⁶-alkylguanine-DNA alkyltransferase. Carcinogenesis, 24 (4), 625-635.
Milsom, M. D., Jerabek-Willemsen, M., Harris, C. E., Schambach, A., Broun, E., Bailey, J., Jansen, M., Schleimer, D., Nattamai, K., Wilhelm, J., Watson, A., Geiger, H., Margison, G. P., Moritz, T., Baum, C., Thomale, J., & Williams, D. A. (2008). Reciprocal relationship between O⁶-methylguanine-DNA methyltransferase P140K expression level and chemoprotection of hematopoietic stem cells. Cancer Research, 68 (15), 6171-6180.
Porto, E. M., Komor, A. C., Slaymaker, I. M., & Yeo, G. W. (2020). Base editing: advances and therapeutic opportunities. Nature Reviews Drug Discovery, 19 (12), 839-859.
Qu, L., Yi, Z., Zhu, S., Wang, C., Cao, Z., Zhou, Z., Yuan, P., Yu, Y., Tian, F., Liu, Z., Bao, Y., Zhao, Y., & Wei, W. (2019). Programmable RNA editing by recruiting endogenous ADAR using engineered RNAs. Nature Biotechnology, 37 (9), 1059-1069.
Reese, J., Lingas, K., Ksenich, P., Sweeney, C., Koc, O., & Gerson, S. (2004). 1006. Preliminary Results of a Phase I Trial Using Retroviral Gene Transfer of G156A MGMT To Protect Hematopoiesis during BG and BCNU Therapy of Advanced Malignancies. Molecular Therapy, 9 (May), S385.
Sakata, R. C., Ishiguro, S., Mori, H., Tanaka, M., Tatsuno, K., Ueda, H., Yamamoto, S., Seki, M., Masuyama, N., Nishida, K., Nishimasu, H., Arakawa, K., Kondo, A., Nureki, O., Tomita, M., Aburatani, H., & Yachie, N. (2020). Base editors for simultaneous introduction of C-to-T and A-to-G mutations. Nature Biotechnology, 38 (7), 865-869.
Schene, I. F., Joore, I. P., Oka, R., Mokry, M., van Vugt, A. H. M., van Boxtel, R., van der Doef, H. P. J., van der Laan, L. J. W., Verstegen, M. M. A., van Hasselt, P. M., Nieuwenhuis, E. E. S., & Fuchs, S. A. (2020). Prime editing for functional repair in patient-derived disease models. Nature Communications, 11 (1), 1-8.
Walton, R. T., Christie, K. A., Whittaker, M. N., & Kleinstiver, B. P. (2020). Unconstrained genome targeting with near-PAMless engineered CRISPR-Cas9 variants. Science, 368 (6488), 290-296.
Wang, H., Georgakopoulou, A., Li, C., Liu, Z., Gil, S., Bashyam, A., Yannaki, E., Anagnostopoulos, A., Pande, A., Izsvák, Z., Papayannopoulou, T., & Lieber, A. (2020). Curative in vivo hematopoietic stem cell gene therapy of murine thalassemia using large regulatory elements. JCI Insight, 5 (16), 1-17.
Watson, A. J., & Margison, G. P. (2000). O 6-Alkylguanine-DNA Alkytransferase Assay. In DNA Repair Protocols (pp. 49-61). Springer.
Wibley, J. E. A., Pegg, A. E., & Moody, P. C. E. (2000). Crystal structure of the human O⁶-alkylguanine-DNA alkyltransferase. Nucleic Acids Research, 28 (2), 393-401.
Woolford, L. B., Southgate, T. D., Margison, G. P., Milsom, M. D., & Fairbairn, L. J. (2006). The P140K mutant of human O⁶-methylguanine-DNA-methyltransferase (MGMT) confers resistance in vitro and in vivo to temozolomide in combination with the novel MGMT inactivator O⁶-(4-bromothenyl) guanine. The Journal of Gene Medicine: A Cross-disciplinary Journal for Research on the Science of Gene Transfer and Its Clinical Applications, 8 (1), 29-34.
Xu-Welliver, M., Kanugula, S., & Pegg, A. E. (1998). Isolation of human O⁶-alkylguanine-DNA alkyltransferase mutants highly resistant to inactivation by O⁶-benzylguanine. Cancer Research, 58 (9), 1936-1945.
Xu-Welliver, M., Leitão, J., Kanugula, S., & Pegg, A. E. (1999). Alteration of the conserved residue tyrosine-158 to histidine renders human O⁶-alkylguanine-DNA alkyltransferase insensitive to the inhibitor O⁶-benzylguanine. Cancer Research, 59 (7), 1514-1519.
Zhang X, Zhu B, Chen L, Xie L, Yu W, Wang Y, Li L, Yin S, Yang L, Hu H, Han H, Li Y, Wang L, Chen G, Ma X, Geng H, Huang W, Pang X, Yang Z, Wu Y, Siwko S, Kurita R, Nakamura Y, Yang L, Liu M, Li D. (2020). Dual base editor catalyzes both cytosine and adenine base conversions in human cells. Nature Biotechnology, 38 (7), 856-860.
Zhao, D., Li, J., Li, S., Xin, X., Hu, M., Price, M. A., Rosser, S. J., Bi, C., & Zhang, X. (2020). New base editors change C to A in bacteria and C to G in mammalian cells. Nature Biotechnology.

### Accession Sequences

Provided herein is a listing of nucleic acid sequences and amino acid sequences corresponding to publicly available sequence accession numbers, certain of which sequences and/or sequence accession numbers are included and/or utilized, in whole and/or in part, in the present disclosure, and/or certain of which sequences and/or sequence accession numbers are included herein as references. Sequences associated with accession numbers are available in publicly accessible databases, as is known to those of skill in the art, and such sequences are provided herein solely for easy of reference.

GenBank Accession No. NP 002403.3 MDKDCEMKRTTLDSPLGKLELSGCEQGLHEIKLLGKGTSAADAVEVPAPAAVLGGPEPLMQCTAWLNAYFHQPEAIE EFPVPALHHPVFQQES FTRQVLWKLLKVVKFGEVISYQQLAALAGNPKAARAVGGAMRGNPVPILI PCHRVVCSSGA VGNYSGGLAVKEWLLAHEGHRLGKPGLGGSSGLAGAWLKGAGATSGSPPAGRN

GenBank Accession No. NG 052673 GTTACAAATAAAGCCCTTCCCGAAAATGGATCAAAAACACTTTGTAATGAGTGAAGGAGCTGGTTTAAAAAGGGCCA ACATCAAACAAAATCCCAGTCCTGAGTCACAGACACTCAAATAGCCCCTTCCAGGGTGGGAATGTCCTCCAAAGCAC TTCCATTTGTGTGAGTAGTTCTTCAAGCCAACGTATCCAAATACATACATCTGATGAGCGGACTTTGCTTGTTTACA TACATTATGTACATTCCTTTGATAGTCAATTGTCTAAAGTTGGAACAGTTCGGAGGGAAGAGCTATATAAGGTTTGC AATGATCTTTGTCCACCAGTTACTGGACAACTGGATCCAAATTCCACAAATAGCTGTGGTTCTTTACCTGCATGGTT CTCCTCCCCTGAGACATCCAGTGCCATGCATTTTCTCTTGCAGGTCTTTCCTTGCATCACCCCTCCAACCCCAGCAG ATATCCTGGCTGAGCCTGTTTTCTCTGTGAGGGGCTACACTGCACACTGGGGCTCTTGAAAAGGGCTTGCTGGGCTG GGCACGGTGGCTCACGCCTGTAATCCTGACACTTTGGTAGGCCGAGGTGGGTGGATCACCTGAGGTCAGGTGTTCAA GATCAGCCTGGCCAATATGGTGAAACCCCGTCTCTACTAAAAATACAGAAAAATTAGCTGGGCATGGTGGTGTGCAC CTGTAATCCCAGCTACTCAGGAGGCTGAGGCAGGAGAATCACTTGAACCCGGGAGGCAGAGGTTCCAGTGAGCCAAG ATGGCACCATTGTACTTCAGCCTAGGCGACAGAGAGAGACTCCAATTCAAAAAAAGAAAAAGAGAAAAAAAAAAAAA AGAAAAAAAAGAAAGGGCTTCCTGCAGGGACAGTCAGTGCAGGAAAGGGCCTCTTTCGTACTCCCACTGCCCCTACG CATCTCCTGGGTAGAGGTCGCTGCTCCAGAGAGCAAACTATTTCCTCATCTGTGTGCCATTCCACTACAAGCCCCAC AGGGGCCTGCAACGAGTCCCATAAATCCATCTCCAGGCCCTGCACCCACAAACTGGCAAAGGAAGTTTCAGGAAAAG TCTTTCTAATAGGAGTCTGTGGGCTGCCTCTGGTTCGTTTCTTTAGGAATTCTCCTCCATGCCCTTCTTGACACAAA AGGGCCTGAAGGGTTCCAGTTCTCAACAGGAGCTCCCAGGGGCTCCTGGCAGGAGGATGTTCTGCCTCTTTGGAGGA AAGCAAGAGCTAAGGAGCCCCTCCTGTTTTAGTTGCACCTCCATCCCCTTTCTCCACCCTGCACCATTCCCTTACCC TCTATGTGAGGCAAAGCCTGAAGTCCCTCCTTCCCGCCCTCTCTCCTGCCCACTAGTAGATTACACTCCCTGCTGCA GTTACACCATGATGCCTCTGCCATGCTGACAACTAGTGTCACACAGGAGCGTAATCAGAAGCACCTGGCATTCAACC CCGGTACCGATGGCCCCCACCTTTTCATTTCAGGCTTCTTCCTCCAGAGTGCTTGAGGCTCCCTGGCCTCCTGGCCT CTCCCAGGTCCAAGGCGCTGACTCCAGCTCTTGGCTCCAACATTTACAGGAACACTGGGACTAAGAAAATCAATTGT GTTAATCAGCAAAACCCTCCACTTGAGATGCTATCCTAAAAGCAAACTATATGTAAAACACTTAACGTGGGGAGCTC TGTAATATGTTTCTTTTGTTACACGACCATCACACATTTGTCCGGAGTTTGGGGCTTTCAAACCACATAAGGTTTTA TGTGATCGTTATAAAATCCCTTCATTTCCCAAAGGTAGGGTAGTAAGGATTAAGTTTGACTACTGAAGGCAGCTTTG TTGTAAGTGGTCTCTAATGTCACAAAAGTAGCTTGGGAAGACAGGCTTATTAAACAAATTTCTTGGGTTGCAAATCA TTAGAAGCAATCGCATGTCATGTTCATTTGCTTAACCCGAAAGGTATCCATTTGGATGTTTAAAGAAACTATGCAGA TTAACATTGTTCCCAGTGCCCACAAGAAAGCTGGCTGATCTTAAAGGAAATCCAGGGAGCTCCTTGAGGAAGTCGGA CTGCAGTTGAGCATACCCTTTTGTGGTTTTCCCACCTCTTTTGTAAAAAGTTCTTCATAGGAATCTGTGTCTGGAGG AAAAGGGGTTGTGATTTGTTAATCTGAAGATGGGAGACCTCAAAGGAATCTCACCCAACCAGTCCTGCACTGTCATC CTATGAACAGAGAAGTCAGGTGGTTGCTCAGTGAGGGGCAGGGCTGGCACCACATCCGAGCCTCCCTGTCCTGTGTT CCTTACTCTTCAGTGGACCCGGCGACCTCCCGGCTTTGGAGCCATCCTGTGTTCCTTTAACAGTAGGACGAACACAC AGGCGATGATGAGATTGTAATGGGACAAGGGGGAGAGGCGTGGGGAGCTCTTGATGAAAACTTGGGTTTTTGAAATT ACCATGATGAAGGGAGGAGCTTGCACCAGCAGGTGTAAGGAGTGTGAGTTTGAGTTCTGCTCATCAGACAGCCTAAG CAGTCACATCCTTGAATCCTGGTCTTTTGCAGGCCAGTCCTTGGTCAGGTGGTGGCCTTTTTCCTCTTTCCCCTCTG CACTGAGACCCTTATTTGGCTCCTGATCCTGGAGCCCCTGACACAGTCCTTACCACTGAAGTAGCTGTGGCCCTTCA CATAGTTCTTCCCACCACGCCAGCAAGGGGACTGAGTTCTTTCAGTACAGCCAGGGCCAGGTCCACACATCTCCCTG GGAAGCCAGCACTTTCTCTCGTGGACCGCCTCCCCATTCCCACAGGGCCCTCCTAAAGGGCCTTTGGATGACCACAT TGGCCTTTCTTTCCTAGGGTGGGGAATTAGAGATGCTAAGACTGGCCAGTCTCTTTTTCTTAAGAGTTTAGGGATTC GTCTTAAAATATTATTCAGATAAACGTATGAGTCATGTGTTAACTGCAAAACTACTTAACAGCACACTTGTTAGAAT ATCAGTCGGCACTCTGATTCCATTTCGGCATTGTTTTTCCTTTGGTTCTATTAAGATCCCGGTTCTAACTGGGTCCT GGTAGTAACAGGCTGTAAGTGGCCAGGTGTATAGCATTATTTCTTAGTAGTGTGGTGTGGTGCTGGTGGTTTATTTT CCCTATGCACACATGCCCTAATATGGAAGGATAGTTTGTACACACGTAGGGTACGTTATCATGAGACTCCTTTACAG AGTATGAAACTGAGGATTAGAGAAGTCAGACGAGTTTCCCGAAGTCTTATAGAAGGCTAATGGAAAAAACCAGAGCC AAGGGCAAACATGTTTCATCTGTCCTTCCACATTAGTTTAAATGTGAATGGCTGCTTGGAACACAGTGTTGAGAAGG ATTCCGAGGCTGTGTCCAGGTTCAGTGGGGAAAAAGTGCCTTGATTTGACCATTATATCGTGTACTCAGGGCCTAGT TTATTTCCCACAAGCAGCTATTTAAAACATGTCTCATCCAAGGCAGGGTCGGGGTGGGGAACCTCAGAGGATCAATG ATGCAGCCTGTATTGTCACCAGGGCTCATCCCAAAGCAAATCCTGCTGAAGTTCTCCTCTGAACAACCCTTATCACT CATGCTAATATGTTGGATCCTGCAAGTCACAAATACGAAGGTATGAGCATTGGCCGAATCCCAACAGGGAAACTTCA CCGGGAAGGTGAAGACCCCTTTCCATTAAGATAACAAACAGAACCTAGAAGGCCCGTAGCATGGTGGCTTAGAACAT GTGGGCCTTGGATGGGATCCTGCCAAGGGGTGTGTGACCTCTCCGAAGCCTCCAGGGATGATACTACTCCCTGGGGT GCTTATGCCAACCACGTTAGAGACAATGGTTTCTGTACCCATTGCCTGGGGCTGCCATAATAAAGTGCCACACACTG AGTGGCTTAAAACAACAGAAACCTATTGTCTCACACTTCCGGGGGCCAGAAGTTTGAAACCCAGGTGTGTTAGGATC CTGCTCCCTCTGAAGGCTCCAGGGAAGAGTGTCCTCTGCTCCCTCCGAAGGCTCCAGGGAAGGGTCTGTCCTCTTAG GCTTCTGGTGGCTTGCAGGTGCAGCCCTCCAATCCTCCTCCCCAAGCGGCCTTCTGCCTATAAGGACACGAGTCATA CTGGATGAGGGGCCCACTAATTGATGGCTTCTGTAAAGTCCCCATCTCCAAATAAGGTCACATTGTGAGGTACTGGG AGTTAGGACTCCAACATAGCTTCTCTGGTGGACACAATTCAACTCCTAATAACGTCCACACAACCCCAAGCAGGGCC TGGCACCCTGTGTGCTCTCTGGAGAGCGGCTGAGTCAGGCTCTGGCAGTGTCTAGGCCATCGGTGACTGCAGCCCCT GGACGGCATCGCCCACCACAGGCCCTGGAGGCTGCCCCCACGGCCCCCTGACAGGGTCTCTGCTGGTCTGGGGGTCC CTGACTAGGGGAGCGGCACCAGGAGGGGAGAGACTCGCGCTCCGGGCTCAGCGTAGCCGCCCCGAGCAGGACCGGGA TTCTCACTAAGCGGGCGCCGTCCTACGACCCCCGCGCGCTTTCAGGACCACTCGGGCACGTGGCAGGTCGCTTGCAC GCCCGCGGACTATCCCTGTGACAGGAAAAGGTACGGGCCATTTGGCAAACTAAGGCACAGAGCCTCAGGCGGAAGCT GGGAAGGCGCCGCCCGGCTTGTACCGGCCGAAGGGCCATCCGGGTCAGGCGCACAGGGCAGCGGCGCTGCCGGAGGA CCAGGGCCGGCGTGCCGGCGTCCAGCGAGGATGCGCAGACTGCCTCAGGCCCGGCGCCGCCGCACAGGGCATGCGCC GACCCGGTCGGGGGGAACACCCCGCCCCTCCCGGGCTCCGCCCCAGCTCCGCCCCCGCGCGCCCCGGCCCCGCCCC CGCGCGCTCTCTTGCTTTTCTCAGGTCCTCGGCTCCGCCCCGCTCTAGACCCCGCCCCACGCCGCCATCCCCGTGCC CCTCGGCCCCGCCCCCGCGCCCCGGATATGCTGGGACAGCCCGCGCCCCTAGAACGCTTTGCGTCCCGACGCCCGCA GGTCCTCGCGGTGCGCACCGTTTGCGACTTGGTGAGTGTCTGGGTCGCCTCGCTCCCGGAAGAGTGCGGAGCTCTCC CTCGGGACGGTGGCAGCCTCGAGTGGTCCTGCAGGCGCCCTCACTTCGCCGTCGGGTGTGGGGCCGCCCTGACCCCC ACCCATCCCGGGCGAGCTCCAGGTGCGCCCCAAGTGCCTCCCAGGTGTTGCCCAGCCTTTCCCCGGGCCTGGGGTTC CTGGACTAGGCTGCGCTGCAGTGACTGTGGACTGGCGTGTGGCGGGGGTCGTGGCAGCCCCTGCCTTACCTCTAGGT GCCAGCCCCAGGCCCGGGCCCCGGGTTCTTCCTACCCTTCCATGCTGCCAGCTTTCCCTCCGCCAGCTGCTCCAGGA AGCTTCCAGAAGCCCCTGCGCGGGCCTTGGCTTGCAGCAACCCTTTAGCATACTTAGGCAGAGTCCCATATTTCCTT CCTGCTGGAGGCCAAGTTCTAGGGGCCTTCTGGTTACTATGGCTGGTGTTTGTGTACATCATACCCTAACTGTATTC ATCAACACTTAGAGTAAGCAAGGCTCGCTGGAGAGCCACACACACTGGGCACCGTAATGTCGGTTATAACACCGCAG AGGAGTTCTGAACTATGTATTTCGCACTCCTGGGTTCATCATCTCCTGAAATCTCAGGGTGGTGTTTGCTCTCAGTT GCTTCAGCTGAGTAGCTGGCTTTCTGTCCTGGAAAGCAGACTTTGTACATGTGTGTGCAACCTATGCCTGCTGAGAT CATCATCAGACAGGGAAGCGGCTTGGTCCAGAGAGCTGTTCTCAGTAGAATGTTAAGCACAGAGAGCTGAGAATTAG ACTGGTTATTTACATAGACATCCAAATAGAAACCTATAGAGTATCTGTTAAGTCAGGCTCTCCCGTCATCTCCCCCA TCCCTGGGCAGGTGTCTAGGAGATGGTTTTGTTATTTTCAGGGCCCTCTCAATGGCTAAAACACTCTGGGAGATGAA CAAGATTTTAAAAACCCAAAGCTTAGCGCACCTGGTGGGTGGGGGTGTGAAAACTTTGAAGGAAACCGCGTCAAGAG CCTGGCTGATTGTTAATATCACGTTAACTCAGAGGGCCAGGATACTTGCCCAGACCCGGAGTCTGCCTGCAAGTAGC AGAGGAGAGCTGGCCTTGCTCTGCCGCGTGTCTTTCTTCCTGGGCCCTCTGTCTCGGGTTGGAATTTGAATAGTGGA GTAGTGTCTGCCACAGTCAGGGGCCTGGATGAAGTAGACCACCAGTACGGAGCTATACTCAGGTACTTTTCAATGTA TATATCTTCATTAAAAAAAAAAAATCTGGGCCAGGCACAGTGGGTCACGCCTGTAATCTCAGCACTTTGGGAGGCCG AGGTGGGTGGATCACCTGAAGTCAGGAGTTTGAGACCAGCCTGGCCAACATGGTGAAACCCAGTTTCTACTAAAAAC ACAAAAATTATCCAGGCGTGGTGGTGCATGCTGGTAATCCCAGTTACTTGGGAGACTGAGGCAGGAGAATTGCTTGA ACCTGGGAGGCGGAGGTTGCAGTGAGCCGAGATTGCGCCATTGCACTCCAACCTGGGCGACGAGAGTGAAACTGTCC TGAAGGGGAAACAAAAAAATCTGTATCAAAATAAGGATTTAACTAATAGTATTAGGCACTACTTTAAAAAGGCTTTG CTGTTAGTAAGGTTGAAAGAAATAGAGCCCTAGGTATACATTGGGTAGCACCTTGTTCAGCTGCTCAGATTCTTGAA CCTTGTTGAGGTCTTACTTTGATATCACCTGGGCCTTACTTTTCGTTGTTTGGGTTTCCTCCCCAGGTGTCTCAATC AGGGCCTCCTCTGTCCTTTTGCAGGGAAGAAGGGGGTAGGAAGGGCCTGAAAAAGAACTTGCCTTCCCCATTGCCCC CTCAGAGTTTGTTCCTCTCCAGTCCTCTTCCCACCCTTTCTTAGGACACCAAAACCCTGACTCTTTCAGTATTTTAT AATAAAATAAAGTTACTAAATTGTCTGTTCTGTTTTGCAGAGGACACACATTTAGGAGCCAGTGATTCAGACCTTCC ATTTTTCTTTTTGGTGCTAATGCTCTTTTCAGTATATTCTCTTTGATAGAAAAAAAATGGAGGCAAAACTGAATTTT CTGCATTATCTTTTCCTTTAGCATTATTCTTGTTTCTGCACTTCATTCTGACCCCCTTCCCCAGCATAAAGTTCCAT GTCCAGATCCACTTGCCCTGAGATTTTTGACTTCCGTTTGGGACTCTAGTCCCTCCAGGGGACACATTGAGCCTCCC AGACCCTGCAGGGCCCTGTGCTCATTCGGCTCTTGGGCTGTTGCTACGGTCCTTCCTGACCCTCTGTTTCCTGGGAC AAGTAGTTTTTAATGCAGCTACTTTCTGAAAGAGCTGGGGTCTTACCGCAGAATATTATTTTATTATTTTTAGAGAC AAGGTCTTGGTCTTTCGCCCAGGCTGGAGTGCAGTGGTGAGATCATAGCTCCCTGCAGCCTCAAGCTCCTGGGCTCA AGCGAACCTCCTGCTTTGACCTCCCAAGTACCAGGGACTACAGGTGTGCGCCACCATACCTGGCTAATTGTTGTTGT TGTTGTTTTTAGAGGCCAGGGTCTCCGTATGTTGCCTAGGGTGGTCTCGAACTCCTGGACTCAAGTGATCTTCCCTC CTAGGCCTCCCAAAGTGCTGGAATTATAGGCATGAGCCACCATGCCTGGCCTCGCAGGATATTATTTAAACGAAACC TGAAGGATTTAAAATCAGAGAAGACACATAAACCATCACCTGACTGTGGGGTGAGAGTGGTCTTTATATAGACACTC ATGATGCACAAGCCAGCCCTTCGCCCCACTCTTGTTTTGAAGTGAAAGGATTTATATGGGAGGGAGGAGGTTTGAGC TGTGCAGGAGGGTCCTTGGTTGGCATGAGGATTTTGTGAACCTACGCTGTTAGCTCTGCAGGCCTGTCTTGGACATA GGTAAGTAGAAGTTGGAGACCTTGTGTTGTGGACCAAATGTTGCAAGATGTGTGTTCTGTGGCTTTTCATTAACTGC TGCATATCTGCTGGTTGGTGGACATAGTCTAGTTTTCTTTTGACTTAAAAAAATTAACAGTACAGCTTATATCGGAG GGCTGCATAGTTTGGGGGAAACTAATGAATATAGTATGGAATGTAATGAATGACTGACTTCTTCATAGAGCTATTGA AGTGATTTATGAAGAGGAAGAAATCATTTTTGTGACTTTGTTGATCTTTGAAGTATTAGAGTGGCTTTGGCAGGTGT GGATCACCCGGAAAGGAATCAAACACTGAGTCCTGCCAAGGTGCTCGGTCCCGGCAGCAAAGCCAGTCCTCAGCAGC CTGCGCTCCCGAAGCTCCTCCAGCTAAGTAACTGAATAGGCCGCCCTGGGAGCTGCCCATGCGGATGCTCTGAATGG TGAAACTTGGTTTTGACACCACAAGTTGTTTTCAGTTAAATCAGTGGCTCAGCAGCAGAGCAGGGTTCTCCAAGGAG GCCTCGTGTCCCCTTCACCCCACCTGGAGGTAGCTGTTCTAAAACCAGTAAAAAATTCTACAAAAACATTTTCTTAG GGTTGTTAAATTAGTCATAGTGTGTGTAACCATGAAAGGAATGGTGAGAGTGCAATAAATGCCTGGTGTGTTCACTC TCCTACTCCTAAAAGTTTCTTGGTAGAATAATTTCCTACCTGTGATGCTTGCGAAGTCAGCAGAGGCTCCCACCCAG CCCTGCCGCCCATTCCCAGAAGTCTGGGCTGGGGGACACCGAATGGATAGTCCACCAGCGGCGTGGCTTGTGAAAGG AGGTACTTGTTAAATCTATGTGTTTTTATTATAGACATCGTTCTGCCAGTCGCAATAATGTGTTTATGGACATTTTT CTGCAAGCACAGCGCGGGTGGCAGTTTTTACTGACAAATTGAGTCATATGCATAGAGGTTTAGAAAGCGCTTGCCTT ATTGACGGATCAGGCAATTCTGGCACGGGATGTTCAGAGATATTTAGGTGGGTGAAGAGAGTGAATGAATCTCAGGG CAGAGAAGGAGATGGGGCATTATATGTTGAAATGCAATAAATAATCGCTAGTTATTTATCATTTGTTTTTAAACTTT TAGCTACATTGTACTTCCCAGTCAAGAGGACTCCGGTGGGCAGCCGGGGATATCATGTTCAGGTGTCTCTCCTGCTT GTGTGGGGTAAGAGCCACTACTGGAATGCGATTCCAGCAAGGCCTGACGAGTCACAGCCCCTTGAACCTGTTGCTCT CCGTAAAGTGGGATCTTCACAGGCAACAGTGGAGGAGCGTTTGAGGGTGACCTGGGCCCTCTGCATCAGAATTACTG TGGGTGCTGGCTAGAGAGGTCTCTCGATTGCAGGGCCCCATGGGAGACCTACAGGACCAGAACCTGGGGATGCCAGG GGATTCTGGCAGACCCTAATGCTTGAGCACCTCTAGGGAAGGCCTTAGGATCCTCTGGGAAGTAGCACTCTCCAGCC CAGGCAAGATCCGAGTCCCCCTTCCAGAAGTATCCATCGGCAGTTGGCATTGCTGCAGGTTTTCTGTGCATATGGGT CTGAGTACGGTTTTGTAACTCCTGAGAGACGTGAATCTCTTCAAATTGCAAACCCAACATGTTATTATTGCTTTTGA GTTTTCAGTTTTGTGGTTATGACAAGATGCAGATGTTACCAAAACTTTGGCGTCAGTTTTACAAACACACGCGAAGC ACGGGCAACCCGTATCATATTTCACCCCTTCTTTCCTTTTGAGAGGGTTGTCTTGTTTTTAATCCCCCTCTGGGTCA AGGCATTTTCCTGGTCTCAGGTGAGGGTGCACTTTCAGAGGCACAGCTGAATACATTTTGTGGGACTGAATTGAGCT TTTCTACAGGAAGTGCCAGGCCGTGTGTGGGAGTGATGCTGTTTCCTAGCTGGGTCCCCCTGGGCAACTCTTAGCCC TCCAGGCACCTCCTCATCCATTGGGTGGGGACGATTATGCTTATTCGGTCTACCCGGGTAAGTATAGGCACACGAGA CAGTAAATACATATTTTTTTAAAAGTAAAATTTCTATCCACTACATGGATAGAAATTTTAATTTGGAGCTGAGTCTC ACGGAAGGATGAAACGAGAAGACGGCGAAGAACCATTTCGAGCCCTCGTTTTCCCCCCAGTCCAATCCGAATCTGTA TTTTGCAGATCTGCACACACCCAAGCCCTCACTGGGGGCATGTGCACGGTGGTCAGAAGTGCACCTTCTGGGAACAG ATGGGCTTCCGTTTGGACCTGCTTTACAAGTTACAATCCTGTGCCCTTGAGCAGAGTTACTTAACCTTCCAAGGCCA GATTTCTCCAAAAGCAGGGCCAGGGACAGTTCCCATCCAGAAGGAGGCCTGGCGCTGGGGAGGCCTGAATGAAGGCG GTTGGTGTTTGCGGATGCAGAGTCGCTCTGTTTTGGTGGAGGCAGGACTGGAGGTCGGGATCACTTGGCCACATTTG CCACCTCTGGTGCGTCTGAGGTCACAGTGCATTGTCTGTGTGCAGCATAAGGTAGTTCCCTATTCATTTACTAGGTC CCTGAAGTCCTTTTGTCTGTAAAGAGGCTACTGGCTCTGCCACCTGCTTCTGTCTTTTCACAGTAGACATTCTCGAC GTGGCAGCATTCTAAAGGTTCAGGTGTGCCACATCGGCCTAGAGTGGGCAGGTGGGCTTCATGTGTGCTCCTAGGGG CACTTGGCAGTTGCTGATAGCCCGAGACACTGGGAGCCTTGACATTATTCATCTGTTATATTGAAAGAACTTCCCAA AGGACGGACACAGGATCTGACACGATGGCAGTGGCCAGGAACCAGCAGTGACACCAGCATGTGACAGTATTTTCCCA TGTGTGACGGCCGTTTAGATCACAGCAGTGAGGGGTAACGCAGGTCCGCTGGGTAAACTTGGTCAAAAATGGGAAGT CTGCAGCATTTCGTGTGTTTGTGGAAATGGGCCTTATTTTTATTTGGGGTGTCTCCTTAATCTCCTGTCATGGTTCA GCTCACCCTGGTGGTTATTCTGGGCGTGCTGCGCTTGTTGCCTTGTACAGACCAACTTAGGAAAGGCTCACCACCAC CCCATGAAGGAGAAGGCGCTGTGAGTGTCTTTATTTTGCATATGAGGAACCCGGGCCTCGGAGAGTTTGAGTGACTT GCACAGGTCTCCTGGGATAGAGGTAGAGTTTTGTTTTCAACCCAGATTTATTTGATAACCAAGTGCAGGCTCATAAC CACTTGTGCCAGGCTGGCTTTGCAACGATGAGCCTGAGAAGCTGTTAGAAGTCCTTTAAGGGGCAAGACAGGGTGTA AACAGACACAGACCTGTTTATGTAGCTAATGTTTTCTGAACATCAATGTGACCAAGTGTTGTCTTTAGCATTGTGGG AAAAGCAATTCGCCCAAGTGTATTCCTGCTTGAAGGCGTTGAGCCGTAGCTGAGGCTGGGTGCAGTGAGGACTTGGG GATGGCAAAGCTGTCCCTGCGTGTGCCTTGTGCCTACCCCGCACCCCACTAGAGAGTGGAACGACCAGGGCAGTGGG GCCAGAGGAAAGATGGGAGGATGCAGAGGGCCAGCCATGTCCCGGGACTGTGCACACGCTTCACCATGTCCCTGCAG GCCAGGGACCCATGCCTGATTCCCTCTGAAACATTTGCGGGACTGTGGCTACCGGTCAGGTTTCTCCAAGTGGCCCA AGGGGTGGGCATTTTGAGCAGAGCAGTGGGACTATGGATGCGGGTACATTTGGGGACCTGGCCATAGTTGAATTTGA GCAGGGTGTGGCAAGCATGTGGGGCTGGACAGGAAGGCAGAGCTAGCCAGTGGGGCCAGGGCCTGCGGAGCCCAAAT GCTGCGCTGGGTGAAGGGCCTTGGGAGTCTTGTGAGTGGGGAACACTAGCCTGTCAGTGCTTCCCAGAGAAGATCAC GGGGCTGGCAGGGGACTGATTGGGGCAGAGGATGCAGGATGGTGGCAGCAGGCCTGGCAGGGCGGGGACAGTTCCAG GAGGTATAGCTGGGGTCAACCCTGGTGGCTTCTGGTGAGAGCGGAGCAGGTGGGAGTCGCAGAGTGTGGTTGTGAGG GACTAAGTTTAGAATCGCTTTTCTTATTGAGAGAGACGAAAGCCTTGGTGTGTGTGCTTTTTATGTGAGGGAAGCCA GATAAAGGATCTGGCTTTGGAAGGGTAGACAGGCCGCCCTCTGCCCTGCCTGGCATCTGGGTTCTGAGGCGGGAGAT TTCCCGACTGCAGCCGCGTTCAGGCGAGGATGGCAGCGGTGGACCCAGGCTCATTACCATTGCTCCCACCCCAGCCT GTCCCTGTTGGGGGCAACCTGTACGTCCTGCAGGAGTCACGGAATTTTAAAAACCAAACCGTCAGGTGTTAGAGGTA CAGTCACATCAGAATGCAAATTAGAAATAGTGTGTGCTGGGGCCTTAAACACCATGACGTGTTTCGTGGGCTGGTGC GGCTGAGTGTGCCCCTGGGCAGCAGAGATTGACGAGGGTGGCTGGGCTTCCTCCGGGCCCTGGGCTCGCAGTGGCCT TGTCTTCAAGGGGCTCCCCGTGTGGTAGGGAGACGTGCTTGTCACACCCATACATTTTGTAGGTACTGGTGAGGGCT GGCTAGACTGAATGAGGTGAAGAGCCTGGCCTTTGTTGGGGGGGGTGTTGGGGAGCTTGGTAAGTGGTTGAAGATGT TGAAGAAGTCCCCTCTGAGGAGGTGGCTTTCGAGTGGAGATTTGAACAGTGAGAGGTGGCAAGGTCTGGGGAGGAGG CTTCCAGGGGACGAGTGAGAGCTGCAGGTGCCTAGATGGCTGCAGGTCGGTGTGTTGGAAGCACAGATGGGAGGTCC TATGAGGGTCACGGGGTGGGGTGGGGAGCATTGAGGCCACTGTTGCAGGCCTGGATTCACTCTAGGAGTGTTAAAGC CAGGGTCTCAGCCTCCTGCAGTTGTCATCTAGTTAAAGTCATGCCAGCAGGGCAGGGCTGTGTGTGTCTGGGTGATG GGGGATCAGATGAGAAAGTGCGTGCCACCCTCAGCACAGTGCATGAGAGGCGTGAGGCACGGCGGATGCTGCGGGTC TTATAAGGCCTTTAACGCTCGAGGGGAAGTTCCTAGAATAATGGGCGAAGAAAAGGAATGATATATTTCTAAAAGTT ACTGAATTTAGCTAGCTAGCCCAAGAAAAGCCCATGTGTGCTATTCTTTATTGTGCAGAAAAATTCGTTTCTGTCAC CATTGTGTTATCATCAGATTAAGCTGTCGAGTCAGATGGATGCTGTGGTTGTGCGGGGTGCGGGCCGATCAGCACAC TCCCAGTGCCTCTGCAGGCCGTCAGCCTCCAGCCCAGGCCAGCCTGAGAGGCAGGCGACGTGGGAAGGGAGGGGCTT CCCACCAGGACACAGGAGGTTGCTGAGGAGGTTGCTTCCATGGTGCCACTTCTGAAATAAGAACTGTTCCTTGGAAG GAATGCACAGGTTCTGAAGTTAGTGTTCTTAAAGTTCAACGTTATCTACCCTCTTGGCCATAGTTGACCACCCTGTG GTCATCCTTGGCCCAGCAAGAGCACAGGCTCTTGGAAAGCCACAATCCATGACCATATTCACAGCTGACCAACCATT TGCCAGGAGGCAGGACTTTCTGTGCTAAAATGGGGATGTCCCAGGCAGACTGGGGTGGGTGGTCCCTGCCTCCCTCG GCTCTCATTCCAGGGTCCTCCTGGACATCACTGCTGTCACTTTGCCACTAGCTTGAGTCCACCTGGGCCTTGAGGCA GAAGCAGTCGTTGAATTTTCTTTTTTTTTGGTTCTCCGGAAGCATGGGCTTATAAGCCTTCCTGTTGTATTTTGCCA TGAGTGTAATTAGAACATCTCCCAGAGCCTGGGTCACTTCCCTTTAGCTGGACATGGCCCCCGCTGTCCTTGTCACA TTTTTTGGATTGTTTGTCACTCCAGAAAGCCTTCAGGGTCATGGTTCTCACTTCCTCAGACATTTTTCTCTTCTGGG TGTGAGGATCTTTTGCTCATAAGACTTGCCTTCTTTGGGCCTGAGACGCTTCTCCCCCAGCTGTGGAGATCATGACA GTTGCAGCAGACGGCTTCTTGGAAAGGTAGAAGGCTCTGGTGTTGGCTGAGGAGAAGCAGCTCCGTGGTCTCCAGGG GTGTCCTGGGTTCCCAGGAATCCCATGGCTCCTGGGGGAACCGCTGCCTGGGGCAGTGCTGGTTCCATGGTCTGTTG GGGTTCCTGGGTGCTCACACTGCCCCAGCTTAGCCGGCGTGACTCAGGATGCTAGGACCCATCGCCAGGCTGCTGGG AGTGGCTGGGTGGGGAGGTCAGCTCTGGCACCAAGGCCATGCTGGGGCTGCAGGGCTTGGCTGGAAGGGGTGCTGCC CTGTGTGTCCTGCTACAAGGGGCCGGGGACCCTCCCAACTCAGGGTGTCCTGCACGCCCACACTGCCTCCCTCTTCT ATCTTTAAGACACCTGGGAAGCTTCTGGACATGTCAAACCCACACCAGACTGAGTGGCAGGGTCCACCACAGGAGTA GCTTAGTGAAGACCAGAAATGGTTTTGAGCTGGGCACTGGCACAGACAGGGTGAGAGCCAGCCGCATGGTCCTCGCA GCCTCCCCGCCTGTGGACCCCTCTCTCAGGCCAGGCAAGTCTGTGGGCTCGTGGGACCCCGGCAGTGCTGGCCTCCT GTGTTTGGGCCTCTGCATCTGTGCTCTGCCCTGAGGTTGGGTGAGTGGGGGACTCGTGTCCCTCTCTTCCCAGAGCA GCCTCTGCTCTATCCTCAGGGAACCTCTTCTGACCCGATCCCCACGCAGACATGCTCTGCTGCCCTCTCTCTCAATC TTCCCCACTCTCACTTCTTCCAGAAGCTACTTCAGCCCCCACGATCTCTCTCTGTCTGGGGCCCTTCACACACCACT GTTTTCTACTAGACTTGTATTGTTTCCATCTCTTGATGGTGGGATGCTAGATTGGATGCTCTCCAAGGCCTGGTGTG TGGTTTCTACTGGATGGAAGGCCCAGCACAGAGTGCTGGACACAGGGTGCTGTGGACTGAACCGTGTCCCCAAGATT CACAGGCTGAAGTCCTGACCTCCAATTTGGCTGTATTTGGAGACAGGGCCTTTAGGAGGAAATTAAGGCTAACTGAG TCATAAGAGTGGGATCCTACTCTGCTAGGATTGATGGCCTTGGCAGAGAAAGAGAGGATCTTTTTTCTTAGAAGAGA AAGAGAGGATCTCTCCCTCCCATACCGTGTGAGGACACAGCGAGGAGGAGGCTGTGTGCAAGCCAGGAAAGGAGCCC TCACTGGAAACGGAATCAGCCAGGACCAGCAGCTCTCACTTGCCAGCCTCCAGAACCGTGAGAAGATAAATGTCTGT TGGTTAAGCCCCCAGTCTGGTATTTTGTTACGGCAGCCCAGGCAAACCAATATATAGGTGAATTACAGACTAAAGTT GTTTCAAAGGCACAAAACTCATCAAGGGATTGTGTATGAAGTTGGAATTTGGGGCTGAGGATAACTTTGTCATCAGA CAGTGCTCCAAAAAGGCACTGTTCTCAGGTTTAGCTCTTGGCATCCCAAAAGATATGAGTGTATTGCAGTAAAAAAC AAGTAACAAACAGAAACCCAGATTTCATGACCAGAAACATCAGTGGTTGTGGGAATAGGGGACAGCAGGCAAGACAG GAGACTCCAGAAGCCAGCAGACCATGGAAAGGCCTGGTGCTGTCCTGGCCCTGGGTGGGCTTGCTCTGGTAAGCGGT AGAGGAACCTGCGCCCTGATGGGAAGGAGAGACCAGCTCAAATGCTGCCAGACTGGGTTTTTAAAACCAGTCTCTAC AGGTTCATTGGGATTCTTTTTGTTGGTTGGTTGATTTTTTCCCCCCCTCCAGATTGGTCAGTATACACACACTAATC CAGCCTGGTTCAAAGGACAGCATGTGGTCGTCTGGTTGGACACCAGCTACATCTCTCAGAACCATACTCTTTGCTGC TTTTCAGGCTAAGATAGCTCTCTGAGACTGTTTGGAGGTCTCTGTTGATTTAGGGGCCTTGCAAGACATTTCTTGCT GAAAGCCCCGCTGGCCCACACCTCACCAAATTGGCTTATGTGGCTACTGATGGGGGGACATAGAAAAGGTAGATAGA AAAGGTTAAATTTACTTCTGTCTAACTCACCAATTCATGCTGTGGATTGTAGATCTGCAAAATATATCAAGTTCACA GGGATTTTATGATTGTTGCAAAGGAAACTCAAATTGTCAGCCTTTTCCTGAAGGATCTTGCCATTCCTAAGCTCTGG CTATGCCCCTACTTCTGGCTGGCTGTGGCCTTGCAGTGCAGGCTGGCACTGTGCCCCTGGCATCTGGCTTTGGGGGG CCCAGCTGTGCAGGGAGCAGATGCTGTGGTGAATACAGGTGAAGCCAGAGTCCTGGCTTGGGGAATGTGTTCTGCTT CCTGTAGTGCTCTATTTGAAGTGTGGATTTCAGTTCTTCGTTATCTTTTATTTATTTATTTACTTATTAGCACAGCT TGTTTGTTGTTCAGTTCCATAGTGAGAATCTCTAAGAATATGATTAACCTTGAGGGTCATCTTATCCTGTAATTATT AATGATTGATTCTGTTTATCCTTGAAATCAATAAGAGAGGCATAATGGTTTGGTCTGTAGTTGGGCGTGATTTCTTA CATTTCGTAAACACGGTTCTGTGACAGCAGTTGTATCAGTTTATTACCCAGAACATGTTTGATGAATCTGAAGTGTA TGATTAGTTTTATATAATTATTGTGATATGTAGTGTTCCTTACATGAGTTATGTTAATATGGCAAATAGTGTTAACA TCTCCTGCCACTAATAGTTAGGGTGGGGACATATAATAATTGATCTTCCTGGGAGTGCTGTAATGTAAAAACAATTC AGGCTGTTGGAAGTATATTTTACAAGTATGCGTTGGGCCTTGGGGATGTTATATTGAAAAACCCAATTGTACAGTTA CCATGGGAACCCAATTAAAATAGATTGGTTGATTTTTTTTTGTCTTTCATCTTGACCAAAAATCTAATGTATTTTAA ATGTTTTCATATGGAATTGTAGTCATATACTGACTTGCTGTGCCAAAGTATTCTGCTCGTTTTTTCCAACTTTAAGC TAAAATAGTGAAAAGTTCTCGAGTGGATCCTTATGAGAACGTCTCCCAAAGAGAATCAGTCATGTTTTTGTGGAAAA GTTTGTGTGAGTGATGATGGTAGAGTGGAGGTGGTGGAAAGCTTTTATGAAAAAAGGGAGAACAAGCATTTTCCTAC GAGGGCAGACAAGAACCCTCATGTTCTTCCTTTGGTGGAGGACATGAGTGTGTTTCTCCGTTCTCACGTTTGTAGAA GGTGAGATTAGGTCCCTAAAGGGCCCTCTCCGTGTTGTGGAGGGGTTGGGGTGGGGGTGGAGTGGTGGGGTGGGATC TGTGTTTGCCCTCCTTGCTGGGGGTCCTCACATTGTTTGGTCTCAGAGCCTTTTTGCACAGTTAAAACTTATGAGGG ATCCCAAAGAGATTACCTGAGTTATATATTAATACCCCATTAGAAATTAGCAGTGAGAAATTTTAAAATATTGATTC ATTTAGACAATAAACCTATTAGCTTTTTGCATAAATAACATTTTAAAATAAAAACTAGCTATATTTTCCATAAAAAC CAATTAGGGAGGAATGTTATCAGTTTCCATTTTTTAAAACTTCATAATGCCTGGATGAATAGAAGACAGCTAGCTTC TCCTGCTTCATTCAGTCTGTTGTGATATGTTTGGGTTGAAGCGCATCAAGAGAATCTGGCCTCACTCAGGTAAGTAA TTAAAAGGGAATAGTGTTTTAACAGCTTCCTCAGATAATTGAGGATGTTCTCCTTTGACACTACATCAGGACTCGGC AAGTGGTAATTTCCTGAGGGTTTGTAGCAGGGTGCAATCCGAGACCCGCTCTGTGAACTTTTTGCTCTCTATGACGT TAAAATCCACAGGTATGCCTTGCACTTTGAGTGATTTTGTAGCGTCTTTCATCGGCCCTGTGGAAAATGTTGCTTCA TTGAGTTATACAGATCTTTCAAATATTGATGTTTCCTAATACAGTCCTAAAAATAATATTTGTTAATATCACCACCT TTCTCCTTAGGAAAGTCATTTAGTATTGGGAAGCCATGGTGGTGGGTACCAGTATTCTAACATTTTAATCATCACTT GAAAGTTCTGGCTGGGCACACTGGCTCATGACTGTGATTCCAGCACTTTGGGAGGCTGAGGTGGGAGGATCACTTGA GGCCAGGAGTTGGAGACCAGCCTGGGCAATATAGCAAGATCCCGTCTCTACAAAAACATTTTTTAAAAAGAAAGTTC CCCTTTTATCATCAGCAGCAGATACTGTCAGTTGTGTTCATTACAGGTACAGACTATTCATTTTTGAGAAAACATCC GCCAGATATGCAAGTCTGAATAACTGTTGTTTGTGAGTCATTCACACTAGCAAAGCTTTTGTTCTAAAATGAAGTTG GTAAGTTCATCAGCAGTCTCTGCCATTTGCGGAGCCCTCCTGGTGGCAGCGTCATTCTGGTTTGTGGCAGAAGTGCT TTGCTGCGCTTCCCGTTAGGTCACAGAATGTTTAGAAGGTCAAGATTTAGTGAAGTTTGATTGCTTCCTCAGGGCAT TCTGAAGTGAACCTGCCTGTTGCCCTGACCGTGCCTTCCTCCTAGAGTTTGGTGTCAATATCTTGGCCCTTGCGGAG GCATCCACGGTTTTCCCCAGCATGGCTTCTGTGTTGTCTTTCACCGTGCGGTGTGTGGTCAGCCATAGTTTGTGCAG GTGCCCTTTAGCAGATGGGGGATGTTTTACTCTTTTCTGCAGTGAATATGGTGGATAACTCTCATTGGTTTCCATGT CTTCAACCAATTTTGTGTTCCTGGATAAATGCCCACCTGGACTTGGTGAATTACTGTTTTCATAGATTGCTGGATTT GACGTGTGGATATGTTTGAGGATTGTGTGTCTGTTCATGAGAGGTGTTGGTCAGCAGCATTCCTGGAGTGTCTGTCT GCTTTGTTAGGAGGGCAATCTTGCACTCATAAAATGAGGCCTCTTCTATATTTTGGATTAGTTTGTGTACCGTTAGC ATTGTCTCTTCTTTAAATGCTTCATAAGATTTACCAGTGAAGCCATTTGTGTTTGAAGTTTTCTTTGTGAAAATAAC ATTAACTATGAATTCAGTTTTTTTTAGTAGCTATAGGGCCATTCAGGTCATTTATTTCTTCTTGAGTGACTTTTTGG GATTTTGTATTTGTCAAGGAATTTGACCATTTCATCTAAGTTTGTTGAATTTATTGGCCTAAAGTTATTTTTATATT CCTTTATTATCCTTTTAATGTCTGTGGGATTTTGAGTCATGTCCTTATTTTTATTCCTGATATTGGTAATTTGCACT TTGTCCCCTTTTTTGCTAACTGGTCTGGCTAGAGGTTTATCTGTTTCTTTTCAGAGAGCCAACTTTCGTTACTTGAT TTTTCTCTCTTACTGATTTCTCCTGTTTATTATTGCTTTCTTGTGCTTACTTTCAGTTTAGTTTGCTTTCTTTTTTG TAGTTTCTTAGGGTAGAGGCTTAGATCATTTGGTTTTCCTTTCTTCTTTTCTCATATAAACATTTAATGCTAAAAAT TCTCTCTAAGCACTGCTTTAGGTGTGCTCACAGATAGACATGCTGTGTTTTCATTTTGATTCAGAGTGGCGTATTTC CCAATTTTCCTTGTGGTTTCTTCTTTAATGCTTGGGTTATTTATTAGTACATCATTTAATTTCCAAGAATTTGGGGA TTTAAAAAATATTTGGGTACTTTCCCAGTATTTTTCTGTTATTGATCTGCAGGGGTGAGTTACCTTGTAATCAGAGG ACATACCTGTTATGATTTTAATCCTCCTAGATTTGTTGAGAGTTGTTTTACAGCCCAGAATATGGTCTACCTTGGTG GATGTTTGAAAAGTGTATACTCTGTGGTTCTTGGGTGGAGTGCTCTTTAAATGTAGTTGGGTAAGGTTTGTTGATAA TGCTCTTCAAGCCTTCTATATCCTTATTGAATTTTGTTTCCCTATTCTAAAAATTATTAGGAGTATTGACATCTCCA GCTATGATTGGAGACTTATTACCCCTTTCAGTTCTGTTGGTTCTTGGTTCATCTGTTCTAAGTGATTTCATTAGGTG CATACATGTTTAGAATTTTAACATCCTCATGGGGAATTAACCTTTACTCTTATGAAATGGCCTTCTCTAGTTTTGTT GTCATATTCCTTTTTTGAAATTATTTGATATTAGGATAGCCACTCCAGCTTTCTTTTGATTAGTGTTTCTGTGGTAA CTGTTTTTCCATTCTTTTATGTTGAACCTGTTCTTGTCATTACATTTAATGTGTGTTTCTTGTAGAAAACACAGTTG GGTCTCTTGCTTTAAAAAATCTAATCTGACAATGTCTTTGTAAAATTGGAGTATTTAGACCATTTACATTTAATGTA ATTATTAATATGGTTGGGTTTAAGTATACCATCTGGCTGTTCATTTGTCATTCACCAATTTGTTCTTTTTGCCCTTC CTCCATCTTCCGTAGCTTCTTTTCAGTTATTTGGATATGTTTTTGAATGATTCCAATTTAACATTCTGTCTACACTG TTGGCTTGTTAGCTATCTCTCTTCTTTTTTAGTGGCTGCTTTAGGATGTGTAATGTACATACTTAGCTTATGCTTAT CATAATCTGCATTCATGTGATATAATACAACTTCACATGCAGAATAAGCACCTTAACAAAAGTATGCTTCAATTTCT CTCATCCCATTTTTTGTTATATTATTGCTACATATGTAATATTCTCCAATATATAGAGTTACTATTTTTATGTTGGG CAATCAGTTTTCTTTTAAAGAGATTTAAAAAATCCAGAATTTACATTTATTTGCATATTTACTGTTTTTTGAAATCC TCATTCCTTTGTGTAGATCCTAGTTTCCATTGAGTACCATTTTTTTTACACCTGAAAACCTTGTACCATCATTTCTC AAGTGCAGGTCTGCTGGTGTCTGAATTGTCATGCATTTGTTTTTGTTTTTTGAAAAAAGCCTTTGTCTCACCTGCAT TCTTGAAAAAATGTGAATTCTAGGTTGACCTGTTTTTTTGCTTTTTAATACTTTAAAGATGTTGTTCCATTGTGTTC TGCCTTGCATAGTTTCTGATGATCAGTTTGCCGTCATTCTTAAATTTGTTCTTCTGTATGTAATCTGGCATTTTTGC CACCTCTGGCCACTAAGGTTTTCTCTTTGTCACTTGTTTTCAGCTGTTTGATTATTTGTTTTTATGTTTCTTCTGCT TGGGTTTCACTGAGCTTCTTGCATCTGTGAGAATGGGTTACATATCCCTTATCTGAAATGGTTGGGACTAGAAGTGT TTCTGATTTTGGATTTTTTTTGGATTTTGGAATATTTGCATAGATAGATAGGTAGATAAATAGATATATAGATAGAT AGATATAGATATAGATACCTTGGGGATGTGACCCAAGTTTAAACATGGAATTTTTGTTTCACATATACCTTATCCAC ATAGCCTGCAGGAAATCTTATGTATTATTTTAAATAATTTTGTGCATGTTACAAAATTTTGACTGTATTTCAACTGA GACCTGTCATACAAGGTCAGGTGTGGAATTTTCCAATTGTGGCATTGTGTTGGTGCAGTTTTTGGGTTTTGGAGCAT TTTGGATTAGGTGTGCTCAACCTTGTTTTACCTCATATAGATTTTTCATCAGATTTGAGGAAATGTCACTGAATATT TCTTCAGATATTTTTCCTGCCTCCCCTTTTTCTGAGGCTCCAATTGCGCATGTGCTGCTTCATACTGTCCTGTGTCT CCCTGAGCCCTGCTCATTTCCTCAGTCTTTTTGTGTTTCTGTGCCTTGATAGTTTAAAAAACGTTATTTTCTCATTG TTGCTATTTTTTCCTTCTGCTGCATCTAATCTGTACATCCTATCGATTGTATTTTTTACTTTAGAGACTGCATTTTA CATCTCTATATGCTCCATATGGGCCTTTTAAAATATCTTCTATTTCTCATGTCATATGTGGGTTCTCCTCTATCTTC TCAAATATGGAGCATGTTTATAAGAACTGTTTTTATGTCCCAGTCCCCTTGTTTTACGCATCTCTACTTTTCTGGGT TTGGTTCTGTTGAATGATTTTTCTCCTGATTGTGGGTTATATCCCTCTGCTTCTCATGTGTGTGTGTTTTAAATGGG ATCCTAGACATTGAGAGTTTTGTTGTGGAGTGCTAGATTATGTTGTCTTTTAAAAAACAGTTTTGGACTTTGTTCTG CTGCAGGTTCAGTTTCTTGAAATTGTTTGTATCCTTTCAAGGTATCATGCTTTTTTTAAAAAAAGGTTTTGTTAGGG CAGGTCCAGACCAGGTTTTAGTCCAGGGATAGTTTAGTCCTACTATTTACATAATGTCTTTACTTTATAAGGGTGTG TGTATGTGTATATATGTATGTGTGTGTGTATTATATATATGTATGTGTGTATTTTATATATGTGTGTATTAAATATA TATGTATTACATATATATGTATATATATGAAAAATAAGGCAGCTCTGAGGTCTTTACCTGATGAATGTTGTATTAGG AGATCTTTCCACACTGGCTGGTGAGAACATGAAATATTCCCAGCTCTGTCTGTGATCAGGAATTATTTGGGCTGCTA CTTCTGGGCATCTTTCTTTGACCTCAGGGAGTTCCTTCTCAAGCAGTGTCTCAGTAGTTAGGCAGAGGCCACAGGGA CTCTTCTGGAGATGCCTGGCGTGTTCTCTGAGTAGGCCCTTCTTCCCTGGTACTCCATCCTGATGATTCTAGGTGTT TTGTTCTTTTTGAATTCTAATCTCTCTCCCCACTTTGGTGAGAGCACCCACTGGTTTTGTGCCAGATGACCCTCCTC CTCCTTGGCACAGCCTGGAGGCTGCATCCAGCCGTTCTCTGGCGGCTGGCTCACTTCACTTCCCTTTCCTTGGGGAG CATAGCCCCTGGCTGCTGTTGTCCTTGTTCTGAAGACCATTGTTTTATATAAAAGTTGAGCACCCCTAATTCAAAAT CTGAAATGCTACAGCATCTGAAAATTTTTGAACATTGACATGCTGCTCAAAGCTGATGCTCAAAGGAAATGCTTATT GGAGCATTTTGGATTTTTGGATTAGGGATGCTCCAACTGTAAGTATTTGCAAATATTCCCAAATCCAGAAAAATCTA AAACCTGAAGCACTTCTGGTTCCCAGAATTTCAGGCTACTCATCCTGGACTTTATTTCGTTTTAGCTGTTACATGTG GTTCTTGCTGCTCTGTCATGGCTAGAAGTGGGATCCCCACCAAGTTATTAGTGGACTCATAAAAATAAAAGAGAGGC CTGCTGGGCTGTTACCAGACTGAGATTGCTCAGATTACACAAAGCCCTCTGGCCCTGACCCCAGACTTTTTACCAGC CTGAAGTGGGTGAAAAGAAAGCTTCACTGACTGCTTTCTAGGCATGTTTAAGCCTGCTTATTCTGGCCCTCACAGAA TAAGAATTCAAAGGACAATAATTTTCAAGAAGACTTAATTTAAGTTCATCTACCTTATTAGACTTGATATAGATAGA TAGCCTTTTTAATTTTTTTTTCTCATGGAAACAGAGTTGAAGAAAACTAATTTACCTTCTAAATTTAGGCACAGCTA ACCAAGTCAAGGTGTGGTTCTGTTCTTCTCTGTTCTCTTCTCTTCTCTTTTTTTTTTTTTTTTTTTGGTGGGGGGTA GACAGAGTCTTGCTCTGTTGCTCAGGCTGGAGTGCGATGGCATGATCTTGGCTCGCTGCGACCTCTGTCTCCTGGGT TTAAGCAATTCTCCTGCCTTAGCCTCCCAAGTAGCTGGGATTACAGGTGCCCATCACCACGCCTGGCTAATTTTTTG CATTTTTAGTAGAGATGGGGTTTTGCCACGTTGGCCAGGCTATTCTCGAACTCTTGACCTCAGGTGATCCACCTGCC TCAGCCTCCAGAAGTGCTGGGATTACAGGTGTGAGCCACTGCGCCCAGCCGGTTATTGTATTTTCTGTATGCTTTTT GGTCGTTCAGCTCATACTCAGTCTTTCCCTGTACTGTTTGAGAAATTCACAAGTGAATCCCAGATCACAGGTGTTTG GTGAAATTGGTATAAACCTTAACTGACTTTCTGTTAAGAAACATAAAAAAATTTATTTGTATTTCTACTCTGAAGAA GGACTGTTCAAACTTTATATGGAGGGGACCCAAGATTGGGAAGGCAGAGGAAACAAAGGGCGTCTTTGATTCACATT ATTTTTCTCTTGTCATCATTGCAGGCTTAAGTGTGTGTCATCACTAGAAATGTATTCTGTTGATGTCTGTCATTTCA GCATTTTCATGTTGTCAAAAATGAGACCAGACAGAAGAAGAGTGTATTCTTTCCTGCTAGAACTCGCACAACACTGT CATTAATGCCTCTGGCAGTCAGGACTTTAATCAGTTAAATGTAATAGTGGTGATAACGTTAAAGCCTTGTATGGCAG TCTACCCGGGCAGCCTAGAGAGCCCTGTGCAACTCTGCATTTGAAAAATCAATCAACTAGGGCTGTCTTTTGTCCTG GGCTACTTGGATAGCTGGTATGAACCACCCAGCACTAGCCTCCTGCTTGTTGAGATGACACTTTTCCTGAAGCCATG CCCACTCTTGACAAAGAATAATTAGGTGCCCTGCATAAGTGTATGGGGACCTGCAGGTGGCTTGGATGGTTTGCCCT GCTCTCATGGGGGTCCACATGAGCATACAGAATGGAAGAAGTCATGATGGTTGCAGTGGTTCCTCAGAGGAACAGTG AGTTTTTCAAGTAGGACTCTGATTATCTGTAAAGGCCAAACTTGTTCCAGTGACATCACATCTCTGGGGTTGCTAGG AAGTAAAGGAGAAAATGGGTTCACTTGCACAGTTTTGGATTACATGTAAGTTTTAAAGAATAGGAATGTGTGTGTGT ATGTATGGTATATACACTATATTATCATTACCAAAAATGATGCCTCCTATAGACTTTTGGTAGAAAGGAAATTTCTT TCTCTTCTGGTTGTCTGAATTTTGTTTTCCTTTTTTTTTGCTTCCCTCCGGAAGGGAATCAAAAAATTGTATATATA TATGTACACACACTATATATGAAATGCCTGTTCATATCCTTTCTGCCTGTGTTTCTGTTGGGATTTCTTTTTCTTAT TGATTTATAGGATTATTTTATATAGTCTGGATTCTAATCCTTTGTCAGGTATATTGGCTGCAATTTTCTTCTCCCAG TTTGTGGCCTGCATTTTCATTTTCTTTATACACTGCTAACCATGTAGGTGTATGTATGTATATATTCTGATTGTGGG TTATATTTCTTTGCTTCTTATGTGTGTGTTTTAAGTTGGATCCTAGACATTGAGAGACTGTGTGTGTGTATATATAT ACCATATAGGGGTGTGTGTGTGTGTGTGTGTGTGTATATATATACACACACAAACACACATAGGTGTATACGCAGGT ATACACACACACACACACATAGGTATACACACACACACACACACACACACACACACACACACATGAATATACCTATA TGGTTAGTAGTGGGAAGCCAAGCTGTTTCTCACTGTCAGATGGTTAAGTTTTGAAATTTTCTCTCATTCTACTCAAG GGTAGAATGATTGGAGTGTCAGTGTTCAATGCTACAGGACAATAACTTTTGTAAAAATGGTTAAAGCAGATTAGACC CCAGTCAGTTAATCCACCTCCTCACCAAAACTTGGAGTTATCATGCTACTAACACTGCTAACCTACTGGGTAAAAAT CCTGTCTCATAGTGATCTTAATTAGCACTTCCGATTGAGAGCAACCTTCAGCATCTTTTCGTGTTGCCATTCATCTT TTGCGAAATGCCTGTTCATACCCTTTCTGCCCATATTTCTGTTGGATTTTCTTTTTGTTATTAACTTATAGGATTAT CTTATATAGTCTGAATTCTAATCCTTTGTCTGTTATATGTGCTGCAATTGTCTTCTCCCAGTTTGTGGCCATGTTTT CATTCTCTTTATAGTGCCTTTTGATGGACAGGAGTTTCTAATTTTAATATAGTTGCATGTAATTGAATTTATGATTC TTTTCCTTTATAGTTTGCACATTTTGTGTCTCGTTTCAAAGTGCTTCCTTATCCCCAGTCATAACAATTCCACTATT TTCCTATGAAAGTATCAGTTTTCCTTTTCATATACAAGTCTGCAGCTCATTTGAAATTAACTTCTGCTGTGGTATGG AGTGGAAGTTCAGTTCTTTTTTAATATGAATAATCAGTGGCTCCAGCCCCATGTATAGAGTAACCCCACCTTTCTCC AGGGCTCTGCAGCGGCTTCACTGCAAATATATGACAAACATGTGACATGTGGATATAGAGCAAGAGAGGCACTGCAG AGCCCTAGAGAAATGAGGAGAGACACAGTAAATGGTGCTGGAGCTGCTGACTATTCCAAGTGGACGCATGCCTTTTT CTAGGCTCTCCTGTTCCATTGATCTTTTTGTCTTTCTGTACTAACGCCACAATGCATTAATTACTATAGCTTTAGGC TAACTCTTAAAATTGATGAGTCAGCCAGGCACAGTGGCTCATGCCTGTAATCCCAGCACTTTGGGAGACTGAGGTGG GCAGATCATGAGTTCAGGAGTTTGAGACCAGTCTAGCCAACATAGTGAAACCCCATCTTTACTAAAAATAGAAAAAA TTAGCTGGGTGTGGTGGTGTGTGCCTGTAATCCCAGTTACTCGGGAGGCTGAGACAGGGGAATTGCATGAACCCGGG AAGCGGAGGTTGCAGTGAGCTGAGATAGCGCCATTGCACTCCAGCCTGGGTGACAGTGCAGGACTCTGTCTCAAAAA AAAAAAAAAAAAAAAAAAAAAATTGGTGAGTAAATCCTCATATCTTGATTTTCCATTTTCCTCTTCAGGAGTTTCTT GATTCTTTTGGGGCCTTTGCTCATCCATTCGCATTTCACAATGAGCTTGTCACATTCTTAGACTAACCTGTTTGGTA TTTTGATTGACGTCCTTGGGTCTGTAGACTAATGTGGGGAGAATAAGATCTTTAGGATATGAATCTGCCTCTTCAGG AATATGCGCTCTTGCACTCTCTGTCTCTCTTGGATTTCCTTAATGTCTCTCACATTTTAATAAGTTTTTTCATAAAC AGCTTGTGCACCTTTTGAAATACTCCCCGTGTCCTTATTTTTTGTTGTTGCCATTAATATTATAAATGGTATCTTTT TCTAGGATTAGATTTTTCTTTATGACTGATATGCGGAAATGCTATTAATTTTCATGTCATCTTTAATTTACTTTCTA ATTTTTTAAAATGAATATACCACATATATATTCTTTGTTTTTTCTACATAGACAGAGTTTTGCTTCTTCTACTCTGT AAACCATTTCTTTCTTTTACTTGTCATATTGCACTAGGATCTCCAGTTCCATTTTAAGTCGATGTGGGGATAATACA CTCATCTTGTTGCTTATTTTAAAGGGAAAACTGCTAACTCTTCATTAACAAAAATGATGCTTGCTATAGACTTTTGG TAGAAAGAACTTTCCTTCTCTTCTGGTTATCTGAATTTTGTTTTCTTTTTTTTCTTTTTTCTTATTTTGCTTCCCTC CAGGAGGGAAACAAAAAATTGTCAGAAACTATTTCAGAGCTGAAGAAAACTTCTTAATCTCCTTCTCCTCTACTGTT CCTTGAAAACTTTTTAAAAATTATGAACAGGCTTCAAATTTTATTAAGTGATTTTTCTGCATCTATTTAACTGTTTA TGTGGTTCCCATCCCCATATCTGTTAGTGTGGATCATGACTTCTAGGGAATTATTAATGTTAAGCTATCTTTGCATT CCTGGGATGAACATTACTTGGCCACTAAGAATCATCCTTTAAACACCTGCTCTAGACCAAGCTTATTAATTTATTGC TTTTAACTTTGGCCAAACTAGTGGTTTAATTGTTTTTAAGGTTATTTATTCTTTATTGTTATTAAGATTATTCTTTT TTAAAAACTGGTTAGGTTAATTATATGAAGCTATATTTTTGCACAGTCTATCTTTTTCTACCCTTTAGCTTTCAGCC TTTGTTCTTATATTTAAAATATGTGTAAAGTAATATAGTTTTAAAAATATCCAGTTTGGTAGTCTTTATTTTTTAAT TATTTAGTCCAGTCACATTAAAATAATTATAGTTGAGCTTACTTTTACCATCTTGCTATCTGTCTTCTATTTGTCCC ATCTTTATTTGTTCCTGTATTTCTAATTTATTTTGCTTTCTTTATGATTAATAGTTTTTAAAACCATTTTTCTCCCC TTTCTGGTTATACAGATTTGTATGTTTCATTTAGTGATTAGCCTAGAGATTATAATATACACCCTGGACTTACTATA GTTCACCTAAATTGCTACTTTGCCACTTCCTGAATAGTAAAATACTTTACAAAATGTTAGTCATTTCCCTTTCTCCC AGCTTGGATGCTGTTGTATTTTAATTATATATACACATTTAAAATGCAGTAGTCACTTTTGTCACTGTTTAAACGTT TGGTGTTTGTGTCACCGTGCCCCATGCTTGCCCTCCAGTGCTCTCCATTGGTTCTCCATCATCATGCCTTCGTCTGG GGTGACTTCTGCTTGAAGGACTTGTTTGTTTACTTAGTTTTTAATGCTAGTCAACTGGCAGTGAATTGGTCCATTTT TAAACACTAATAGTTATTGATTGTTGAGTTAGCAATTCTTTACCTTATAAAATATGACATTCCATCGTCTTCTGGTT TCTGAAGTTTGCTGAAAAGTCAGCTGTCAGTCTTATTGTTGCACTTTTGAAGATAATGCAAATGTTTTTACTCTGGC TGCTTTTAAGATTTTTTCTTTGGTTTGAAGCTAATTGTGTTATGGTTCTTTGAGGTTTTCTTCATATTTATTCTGCT TAGGGTTTGCTGAACTTCTTGAATTTCTGGATTGCTCTCTGTCATCTATTGGCAAATTCTTGACCCTGATCCCTTCA AATATTGCTCTGCCTCATCCAATCTGTCCTTTCCTCTCGGAGTTGAATTATAGGTAGGTTAGTCCTTTTGACTGTCC CTCGGATCTCTATTCTTGTTTTCTTTCCATTCCTTTCCCCTTTCTTGACCACAGTTTGGATATTTTCTGCTGAGCTC TCTGGCAGATCACGGACCTTGTTTTCTGATGTGTCCAGTCTGTTACTAAACCCATCCAATGAATCCTGAATTACAAA TTTTGTATTTTTCAGTTCTAGAATGCCCGTTTTGTACTTTAATATATATTTTAATTCTATATTTAAAATTCTTCTCT TTCCCACTTATGTTGTCTGTCTTTTCCTCTTATTATTAAGTGTATCCATTGTAATTATTTTACACTTCTTATCTGCT AGCACCAGTATTGTGGACATCTATTCCTCTGCTTGTATTTTCTTCTTTTTTCCCTTCTCTTTATTATTATTAAATAC ATGTTTTTGCTTTTTGCATGCTTTTAAAATTGTGAGTGTTTGCTGGACTTGTGCATAAAGGACTGTAAAACCTCTGG ATTGTCGTGTTTTTCATCAAAGCAAAAGCTCTCTTTGCCCTTGGTTAGGCATATGGAGTGAGGGGTGAAGGACCTTG GTTTCATGAGGGATTGATCTGGGTCAGGGCTGGCCTGTTATTTTACTAAAACTCAGTCCCTAGTTGGACCTTCCCTT TGAGCATGGTCTTTTGATTGGAAGCCCATGGTCTCTTTCTCCTCAGCCTGTAAATGCTTTGGGAGATCCCTTGGTTC TCCCCTTAGAAGGTTTTGAGCTGAGCTCTTTAGCTTCCCACGTCCCCTGTTTCAAAGTCTCCTAAATGTCCTGATGG GAGATGTGACTATGTGTGAAGCAGGCCCTTCTCTCTCTGTCTTTCCGCCTACCTTTCCTCCCACCCCAGTCTCGTAA GTGCTATTAGATTAAGGGATAGTGTGCTTTTCCTTTTAGGAGCCCTCACTTGACCCAGCTCTCTAGGCTCATGAACG CAGCCCCATAGGATAGCAAATTGATTGCACTTTTGGGGTTCTGATTCTGATCTTTCATGCCATCCCCAATGGGCCAC CCAGAGCTTCCCTGGTTTCCCTTCCCAGGTGGAGTGGCTTTGTGTGGACTGCCACGGTGCTCCCCATCTGCACCCAG GATCAGCAAGTGTTTCTGGGCAGCAAAGGGCAGTCTCCACTGTACAGTCCCAGTCCCCCTAGTCCTGGTTGCTTCCA CAGCTCTATAACATTTTAAAAGACGAGTTTTGTAATTTTTCTGACTTTTTCCAGTTCCTGTGGGAACATTGGGCTGC CTCCATTAGTTCGTTTTGCTTGGAAATGAAAGTCCATTCTGAGCACTTGCTTGAAATCTTGATTGAAGTCTTTGATT TCTGCTTTCTCCTTGGTCATGCTTTCTCCTGTCATCTATCTTCCTTTGCCCACACATTTCATAAATGCTTATTTCGT ATTCTACACAGGTTGCTTTAAATATCTGCAATCCTGTGGGTGGAGTGTGGGCTTTGTGGTTTTCATCTTTCTGTGAT TTCTTGTAACTCTTACGATGACACGGGACCTTGTTTGATGACTTGGTTGATTATTAATTTATATGGGCTGAGCCTGA TCTGTTTGACTCCTGAGTGCCTAAATTAGGGATGCTTTTCCCTAAGGGTGGGTGTTTGGGTTTCTGTTGCCTGGAAC CAGGGGGTCTCAAGGCAGCACCACTCAGATCTAGGACTTTCTAAGGCTTCTCACTCAATCAGGGCCCTCGGGTTGTC AGGGAGGCCCCAGTGGAGCGTCTGGAGGTCATGAGGTGTGCACCTTCTCTGCTGATGCTGCCCACTTTCCCTCTGCT TCCTCCAGCCCTGGCTTACTCTCCTTTTTCCAAGTGCCCCGATTCTCTTTGGATTGCCCCTGCTTACTAGAGAGGCC ATTCAGGAAACTTTGAAACATTGCCATTTATTCAATATCTAGTGCATCGTAGTCCCTTAATCCAAAACAACTGGTTT GGTTTTTGCATAAAGCTACCAGAAGATAAATATAGATAAAAATCTTGAAGATTTAGAAAAAGTAATATTAAAATAGA TCCTTAGATATGTTCTTATCAACATCTGCAAGTGTTCATCCCAATATGGATTCCATCAGTTAGAGGGGTTTTAAGAG GGGTCACTGTGCGCCAGGCCCAGTGTGGGATCTGGGGGAGGTTGTCAAAGTTGTCTTCTAATCCAGAACAACTTGCT CACTAATATTTATATTTCTGCCTTCAATCTGGGAAGCAGCCCTTTTCAGAAAGGTTTGCTGCAATACTCTCTAGGGG ACAAAGCTGAGTGAAACTCTAGAGGCTTGTCTTATACGCATAGACATCACGTGCACACTTACACCCCAGTCATTTCC GCATGTGGGATCATCACAGTTGTGTGTTTGACTTTTTCACCTGACCTTGTAAGTGCTTAGCCATATATTGAACTCTT TAATATCATTTTTGGAGATGGCATAGTGTCCTACTGTAAAGATATAACCTTCTTCCATTGTTAGGAATTTAGGGCAA TTTCATATCTGAGACTCTGTCTTAAAGAATTAATTAAAATTTTGTGCACAAAGATGTTTAATATAGTGTTTTGAGAA GTCAAAACACAGGGAAGACTTCTTGTGTCTTACACTGAGAAATTCTTAGCCAGCCTCTTAGTTTCCTGAATTCAGGA CTGTGTTGTGTAATTTTGAAGCCCCAATAGTGATATGGTCATTTTTTTCATATCTTCATTCAAAAAACCTCAGTCAC CTGGGGAGTGGGTGGGGTGTGTGCTGGACCCTGACCTTGAACTGTCCAGACAAGTTCCTAACCACGTGGAGCTCAAG CTTCCCTGGGGCTTACCTGAATGACCTGCTCAGTGGCCAGCCACTTGCTGCGTGTCAGCCGGTGAACTCTGGGTTCC TGGCTTATCAGAGACTTCAAGTCCTCATTACCTTCTGGGTCTCAGTTTCCCTAATATGTGCAATGACAGGATTGGCC AGGGCTTTCCAACAGAAGTTTTTGCAATGATGGAAATGTTCTGTGTCTCTGCTGCCCAATGTGGTAGCCACTAGTCA CATTGGCTGCTGGGCACTTGAAATTGACTCCTGTGGCTTAGAAACTGAATTTTAAATTTTATTACATTCCAATTAAT TTAAATTTAAATAAGTAGCCACATGTGGCTAACAGCTGCGTATGGACACTGCCAGTCTAGACGGATATGATGCTTGT TTAATGGTCTTAAGAGGTCTTTAGAGTCTATTCTAATCCTGACATCTGGTTTTCTTTGCTTGTTTGTATTTATTTGT TCCTTTATTTTTACCACGACTGCTCTGAGTACCAGGAACTCCTTTTTGGGATGTCACAGATCACCAACTTCCTTCTC TGTGCTGTGTCAATAGGGACGTCACCCCTGTGGGGAAAGGAATTTGCCTGAACAAGACACTAAAAAGATAAAATAGG CTGAAGCGGAGAAGAATAAAAGATTGTGCTGGCATGAGCTAGTTTTGCAAAACCCATTTAAATAAACTCAGGATATT ACCGACCCCATGCACACATTCTTTCCTTGACTCTGCAATGTTCCGTTAAACCAGATTGTGAATTTTTTTCAGGATTA ACAATTTACATTTATGATTCTATTTTCTTTTATAAGTAACTGCTATTTGTAAATGAGACTAATTATTTGAATTCATG GTACAAATAACGTAATTTAGAGTTACAGAACTCTTGATCCTCTGGGTCTAAGCAAAATAAACGTAGTACCTAATGCT GGAATAGAGCAAGAGTTTTCTTTGAATCTCAAAATATCTGCATTGTGAAACTGGGTTAAGTTATTTTGGAAACTATA AATAGTCACTTGGTAATTAGGTGTTTGGTTTTATTTGTTCTAATGGAGTGCTTATTACAGTCGGTGCATTTTTACCC CACGGATCAGAAGCTTTTATAGGCCAAGTGATGAGAGACCATCTTCTACATTTCATTTGCTGATTGTGACTCATCAC TGATGATCGATGGACTGCTGAACTTTTTAATTCCTGTTTGCTTGTAGAGAAAGAAAATTCTCATCTCTTTTGATAGG TCCTACTCCAAAATTCTGCAGCCGTTTTGTACAATCATAACCTTACACGATATCACACAAAGGCAGAGAAACGATTG CATGGACATGCCTTCCTGTCAAACGTGTATGGAATCTGGCCCGTCCTAGGCTTATCTCTTTCCACCAGGATTCTTCT TCCCATTTTTCTCAGCAGAGCATCTTCACGTAGTTCACATGACATCAGAACTTCATCAGGATGTAAGTTCCTGCAGA AATAGGCCTGTGTCTGGTCAGTAAACAGAGAATGTAATGACATTCCCAGAGCATTCACAGGCCAGAGTCTGGTGTCC TCCCCAGCGTCCAGCAGGCTGCGGATGGATGGCATGCAGCTGACAAGATTGATACTGCATTAGACATTCCTGGAGGT CCCTGCGGCTGGGGACTGGCACTCGCAGGCCTCCCCCTGCACACAGCTGTCTTTGGAGAGCACAGAGCAATGGGAGG CGGTTTTGTTTTACTATTTGCCATTTAGAGTCTCACTTTTAATTTATCCTCACTCTTTAAGAAATAATTGGTAATTT ATTTCTTAGAGATCAAATGTTGCTACAAGCTGAAAACTGTTTTATAAATATTTCAGGAAACTTGAATGCCTGGAATT TTCATCGTCACGCTTAAGTCTTCCTCCAAAAGGGGATGGACTTCGTTCCATCCCGCTCCAAAAGCTGGTCCGATTCT TGCCTGGGTGTTGTATGCTATTTGCCTTTTCCATAGGCTCCAATAACAATAAGTACATCTTAAAATTGATGAAAGTT TAATGAATGGCTAGCAAGCAGAGAGACTACTTTTATAACAAGATTAATACCTTGGCTCAGGGGACATAACATTTTAG TAAAATCAAAATTTGTATAGTTATCCTAGCGGTGCTTGCAACCTCTGGCTTCCATATAAAAATGTCTAAATAAAATA CTGGCAGCCGTCTCTAGCCATTATTTCCATCTGCTCTAGGTGGCCTGACCCGGAACCCACTCAGGGTGGTGCACTCT GGGCCGGGGTCTCTGGGATTTCTGTCGCATCAGCTTGTGCAGCCGTGGAATGGGGATGGGGCACTGAGGCTAGCTGT CCCGTGCTCTAGAGGTTCCTGGGAAAAAAATACAGAGAGAAATAACTCACAAGGGAGCCAGCATTTGGTGTTGTTTT AAATAAATTCATTCATCATCTATACCTTTTCCCCTTTTAATTACTACACTTTAAAAGGGATCTTGGTATATATTTAT TTTTGTTTTGTTAATTTTTTTCTTTTTTAAAAAATTAATTGTAGAGATGGGGTCTCTCTACATTGCACAGGCTCGCC TTGAACTCCTGGCCTCAAGTGATCCGCCTACCTCAGCCTCCCAAAGTGCTGGGGTTACAGGCATGACCCACTGTGCC TGGCCAGTTTTTTTTCCCCCTTTTTGGCAAGGGAAGGCTATCGGGGTAGAAGTTCTGAAGCAAGTACTCTGAATGGT TTCTGATTTTAATAGGGGGAGCATCATTTCCTGATCTAATTTATAATGTGATATAAAGCTGCTGGCCAGCTGACACT AATCTTAATTTATGATCTATTTAACCTTTATATTTCCTAGCCACAGCTCGTACCACTCCCCCGTCTCTGCACCAGCA TGTTTCAGAGGCAGGCAAGCAGCATCAGTTCTGGATCCTGGAGGCAAGGGGAGGGTTTTCACGGAGGGGCGGTGCAC AGCTCTGTGCCTCCCCGGTCAGCTGGTCCGGCACTGCAGTCAGCAGCAAGGGCGTGGTGAGGGCCCTGCAGATTCTT CACACTGTCCTGGTCCTCTCTCCTGTTTCTGAATGGCAGGACTTTGGGGTCAGATTAGACCCTGCATGTACATAGAT GACAACGGAATGGCTTTTTGGGGGGACTTTTTATTTTGAGATAAATTTTAGACTTGCTGAAAAGTTGCACAAATAGC ACAAAGTTTCTGTATGCAGCCACCATCTGGTTTCTGCCAGCACTAACACCTCACACTTACCACAACCAGGGCGCTAA CACGGGTGCACTACTGTAAACCTTGCCATCTCTCCTTTGTCTCCTCTAGTGCAATGGTCTGAATTTATGCTTCCTCC AAATTCCTGTGTTGAAACTCTACCCCCATGGTGATGGCCTTTGGAGGTGGGGCCTTTGGGCAATGACTAGGTCAGGC TGGAGCCCTCAGAATGGGATTAGTGTGCCTATAAAAGAGGACTGAGGGAGCTCACTGTCCTCTTCCACCACGTGAGG ACGCGGCGAGAAGGTGCTCTCTGTGAACCAGGAAGAGGGCCCTTATCAGACACGGGCTCTGGCGGCACCTTGATCTT GGACTTCACAGCCTCCAGAATGTGCGAGATGAATTTCTGTTGTTTACAAAGCACCCAGTCTAAGGTATTTTATTCCA GCAACCAGAAGGAACTAAGACGTCTAACTTGTGACAGTTCCTCTTGCCTTGTTTTTTGTGACCCTGGACCCTTTCGA AGAATACTGGTCAGGTATTTTGTAGGATGCCTCTCGTTTGGGTTTGCTGATGTTGTCTCACAGAGAGACAGAGGTTA CGTGTTTTTGGTAAGAAGGCTCAGAAGTGATGTGCCCCCTTGGGCCATCCTGTCCTGGGGACATATGTCTGTCAGTT ACTGTGATGTTAGCTTGGTCACTGGGTATGGTGAATCTCTTGTGCATTTCCTCTTGGTAATTGAAGTTTTCCCATTG GTAATTAATACCTTGGGGGAGATATTTTGAGACCATTTTGGTTCTGCTCAGGCTTTCACCCACTCATCTTAGTGACC GTGGGTGTGTCTTGCCTGTGGCAGTTATTACTGTACTGTTTCCCTAATGTTTGTATTTCCCTCCTTCCCTTTTCATT TACCAACGGGAACTGTTCTGTAAGGAAGAGCTGTCTTTTCTTTCCCTTTTATTTATTAGTCATTGAATTATTCACAT CAGCGTAGACTCATGGATATTTATTTTAATTTCTGGGGTATAGTCCAGTGCTGTCTTTACTTAATTTTGTTACTCAA ATAGCCCCCAGTTTGGCTTTTTGGAAACTCTTGGGTTTGGCTCCTATGTCTTTTGGACCTGCCCATCGTTTTTCTAG CGCTTTCCTACTTTCTGGGATCATAGTATGATCCAGGTGTATCTTGTGTTTTCCCTGCCCCAGCCCTGGATTCAGTC ACTTCTCCAAGGAGCCTCTCTTCCTTTTAGCTGGAGGATGATGTTTAGCAGCCAAGATCTGGGTGCTGAGTGTGCTT GCTGCTGCTGAGTCACTGCTCTTCCTGCCCTTCTCAGTAGGGGGAGCTAGGGGCTGTATGTGTGTCTACTGACCCGT GCATCTGCATTTATTTCTGTGTCTGCTGTCTGCATGCATGTTAAAAACTGCAAGTTCATCTGATGCCTCTAACTTGA ATTCATCACAGCAGGTTTTATTCTAATCTCTGCCTTTCCTCTTTGTAGCTGCCTTCTCCAACATTGAGAAACCTGGC CCTCTTTATCTGCAACATGTTTGCTTATATGGTGGCTTCCTTTAAAAAGAATCTGGTAAAACCTGTGTCATTGTGTT TCAGCCACTAGATGCAGATGAGGCTTAGTAGGAAAAGCCCTACTTGTGAAGAACAGTATTTCCTATGCTTGCTTGGT GGAAAAAGTCACAGAATTTTCTATAGAATTCTCTGTAAACATGGTTAGGAATATAGGGTTGACATTTGGACTCAAAT AGAAAATGAAATCCTTTTGTTTCATTACTTATTTCGTTACTCAGCTTTCACCTTGACTCTTGGAAAACTGATGGCAT GTTTCCAAATGGATATGCCAGCTAATTAGCTTATTTCTCAAATATCCATGTTGGATGTGGAAAACAAATAAAAATAA AGCAAGGAAGACCCTCTACTGTTGTTATGTGAAGAATAACAATTACCTTTAATAGCATTTTCTGCTAATTTAGAGAT TTTTCTTTTTCTTACACTTATTTTCTAACTCCTATGATTTGTGTAATTCATATCTTATACCTACATCTGATTCATTA TGATTCTTAATTCTCTTTGTTGTAAACAGAAATATTGAAAATTCATTCGATGAACCTAAAACCCTTTAAAAGGTCTG GTTAAATTTTAACATTAAGGGTGATACAGTATTCATGCTATAGAACACAGACAGTCAGAAATCTTATGAAGTGTTGG AGACTTTAATGGATACTGTACTATGTAGAATCTGATCACGAGACTCCTGCTTCATCACGTTTTTACTCGAATTGGGT TTCCTGGTGACAAAAGACTGTGAAGTAGCTGACGGAAATACATCCTTATGTCAGTTTTCACCCGATGACTGAAAATT GTTGGATATATGCGTGAAATAATTAGGTCTTATTTTTGGAAGGGAGAGAATATAACCGACTGAAAAATAATTTGGAG TTAATTGTCCTGAAAATTCACCTACTGTATATTAATAGGTTAGGTGTTTCCTAATTCTTTCTTTCTTAAGCATATCT TCCTTTTCCTTACACTCTTTTTGACACAAACTTTTGAATATGGACTCACATTATAAATCTTGGGATTGGAAAGGCCA TAAAGATTGGATTTTTAAAACTTCAACAATGTAGAGGAATACCAGTGTTGTATTTGTTCAGAAAGTCCTCATTGGCG CTGATTTGGGCACCGCCTGGTTTCCTACCGTAGAAGCGTGTGCTGTGGTGGGGTGAACTGGGGGGGGATTCACACC AGCCTGGGCCCTGGGCCATGGGGGGCTGCCTCCCAGGGCTGCTTCAGCCGACAGGAGGGTGCGGTGGTGAAGAGGCA GGGCTCTGGGGATTGGAATCTCCACGCTTGACTTTAGGCTTCTACTGCTCACTCGAAATGGGACCTGAGGCAAATTA TTCACCTGCATTGTGCTGGGTTCCTCACCTGTGATACATGGATTATAATATCATCTCTCTTCATGGGTTATTGTAAG GATTGATTTTATTAATATTTCGAATGTTAACGTTATTATTTATTTATTTTGAGATAGAGTCTGGCTCTGTTGCCCAG GTGCAGTGGTGCGATCTCAGCTCACTGTAACCTCCACCTCCTGAGCAATCCTCGCACCTCAGCCTCCTGAGTATCTG GGATTACAGGTACCCACCACCACACCTTCCTGATTTTTGTATGTTTTTGTAGAGACAGTGCTTCACCATGTTGCCCA GACTGGTCTTAAACTTCTGAGCTCAAACTATCCGCCCGCCTTAGCCTCCCACAGTGCTGGGATTACAGACATGAGCC ACCGCACGCAGCCCAAATGTTAAGTGTATGACATAAAATTCAGGGTTACTTTGCCTAAACAGTTCAGTGTCTCTGAT TAATGTGATTTCTGTTTCACTGACAGCTTTCATGTAAATTTAAAACTAGATAGAATTACAGAGCTTCAGAGTTGTAC AGACTTGTAGAGGTAAAGTAAAGCCAAAACATTGAGCTCAGTGACGGGGTCAGTCACTTCCTGGTGTCAGAGTCCAC TGACAGGCTCTGGCAACTCTTGCATCAAAATCCTTTGCACACAGGTCCTTTCTTGCTCTTAGCCAGTAGCAGCTGTC CTGGGCAGAGTTCCCTCAGTGAACCTGGAAACGGCAGTGGCTGGAGGTGAGAGGTGTCCATGTACTCACTTGTTCAA AGTTGCCCCCTCTTCCCTCTCTGGGCTTGGGCCCTGATACCTCTCAGAGCTACCCTGCCTCCTGCGGGGGTGGGGGA GGATGCCTTTATTATCTCTAGGCCAAAGGTTGGACCTCCACATATGTCCATGATGAGGGACAGCAGTCATCCCTTTC AAGACCGAGAAAGTATTTAACTCCTTGGAAAATTGAGGGTTTATTTTGCAGTGGTTTGGAAAATGTGCTGAGGGTGG CTGTAGAGTTAACTAAATAGAATGTTTCAGAGTCACAGAGATTTGTAATTGGTGGGCTTGGTTTGTTTTTATAGTGC TTACTCCTTTTTATCTGCCTTGTGGATTTATGCATTTATTTGATAAGTCAGTCATTTACCTCCCCATTCTTTGGTTT TCATGTTTCTGTTTATCATCAAGATAAGCAACTGTGGGTTTTCTTCCATCGAAGCACCTGCCGCAGGTGATGATTTT AATGGTATGACTGCCATGGTCACCACGACCATTCCGAGGACTCTAAACATGTTTCTTGTCCAACACTACTTTAATTT GAAACAAAGCCTGAATTGAGCAGCATTCGTGTCTGTGGTAGACCAGGGAGGCAGGGCTCACCTCTGTTTTCAGTTCC CCTTTGCTGGATGTGCAGGAGGACTGCCCCGTTCCACTCCTTCAAGGGTGGCTTGGTCATGGACTTGGTTTGGCTAA TGAGCGTGGCCTGGAGTGGCATGTGGTGCTTCTTGGTAGATGTGTGTGCACACCAGCTCTTGGTTCTCCCCATGTGA TACTGGGACAGCTGTGGGATGGGATGGGCGCAGCCTGTGGCATCAAGCTTGCATGAATCAAGGCCGCTGCTGGGGCT AGCCCAGACCCGTAGTGGGTTTGGTGGTGGTTGTTACTGCAGAGCGTCCCAGCCATCCTGACTAGTACAGCGTGGAG TGTGTCTATGTCCTGGAGCTTGGGGAGAGTCCTGGCCGCAGTCCAGGCTTTGAAGAATGTTGACTTGACCTGGAATA CCCACCTGTCCCTGGAGTGCTGTGTTCTTAGGCTAGTTCCGTAACCTCCCTGTCTGCTTCTTCATCTACACACCATT TGATCTTCCTTAGGTTTGCTGTAAGGAGTAAATGAGATAACGCATGTAAAGCACCTGGCCCGGTGCCAGGCATATTT AGTTAATACGTTGCGTTCCTACCCAGCTAGACCTTTTAACAGTCCATTGGCATCAGGTCACCTACAAAAAGGTACAG AAGCTGCCTTTCTCTGTGGTATTTGCTTTTTGATTAAAAAAAGGAAATAAATGATCACATAATTGTTTTATTTTGCC TTAAAAAATGAGTAAAAAAAAAATGTGGCTCACATATGCCAGAGGTGTGATATGTGAGTCGTCTCTCCCTGAGGAAG GCTGCAGCTGAGGTTGGGGGTGAATTTTTTCCCCTGCATGAAGAGGGGTGACTGGGGATGGGGCCGCCTCCCTTCCT AAATTTCCGTGCTCCCTGTTGGTGTAATTAACTTAGGGGAAACCACACGTGGGGATTCAGGGGCTTCTCCCTTTATT CTTTTCCTTAAAATCAAGTTACATTTTTTTTTGAGCAATAAACTTTAGAACAAAATGAGGAATAGAAAACAAGATGT CGAGACAAACAAGCCTTAGCGAGGCAGCTGTTAAAAGCAGCCGGACCCAAACAGAGCATAAAAATGCATGTAGTCCA GAGAAGCACTTTTTCTTTTTACGGCTCTACCCCGTATTTCATTTATATTTAAAACTGAGTTGACGAATATGAATACA GATATTTAGAGTTATTGTAAAGTCGTATTTGTTTATGTGCGTGGTCCAGTTTCCAGGCCATATCTTACAGTTTAAAA GGGAGAGCAGTTAATGGTTTCGCCCACACTGCTTGGATTTTTTTTTTTATTATTATCCTCCAGCTCCTATTCTTCAG GTGTGCAGCTATTTTAGTATTATGAAGAATGATGGATGATGCAGGAAAGTTTCTTTGGAAGCACTTCTGATATTGTG CAATGCACTTGGGGGTCAGCTACACTTGACTGATGCATGATTTATCCAATGTCCCTGCCACTTCCCGAGATAAAGCT GCTGCTTTATCGAAAATGCCAGCAACTGTGATTTAGAATTTATAAACCTCCGTTTTCAAATAATTGTTCTGATTACT TTCTGCCTTGGCAATAAAATGATAAAGGTAATTGCACACAAATGTGAGATAGCACCAAAATACAATAAAATTAACCT GAATTAGCTTGACTTGTCGATGAGAAGTATTTATGAAATGTGGTGTTGCAATAAGGGAGGGAGAGTGATATTATGGC TGGAAGGTGAGTTATTGCTTTAATGTGATACTCTTTGAAATTTAAAAAAGAGACTGTTTCTGTTGAGGGTGATTGTG CTTTTGAAATTAAGTTGAAGTGATTCTTATAATTGGCAACGAGAATGCATTTTGAAGGTAATATCTTTTATTGTGTT ATGCCTAGTTGCATCACCTTTTATTTAGCTGTTTCTGGCTTCAGCTTTTCTAATACTCAGGTAGAAGCAGACTTGGA ATTCTGTCGGATCCAGGGGCTGAAATGTTGTATTCTCTTTGTAGCAATGCCCCTGGTCTTAATGTTACAGACTTTTT CCTCCTACATTTGATTAAATTGTTTAGATATCAAAGGTTAAATATTATAGCTAGTTAATGTTTTTTTAAGTTTAAAG GCGGTTTTAGAAATGTGTTTTGTTTCCCTGTCTTTGTGTGCACTGGGAACATCTCATTGTAGCCTGATGGTTTCAAC AGTATCAGATCAGGGTTCCATGTCTGATACCCATAGTTCGTAATCTGCCTTTTACATTCCTGAAATGAAATTCCTGG ATAATATCCATCTACATGTACTATTTAAATGAAACACTTTAGGAGCAGTAATTTATAATAAAATAACATGCCTTCAG AACAGGAATGCTTGGGCAGGACTCTGCTAGCAGACATAACGTGGCAATCAGGTGTATGTACCTGGAACACAGCAGCT GCAAATGGAGATTGAGACAGTTTTGTTTATTGGACATTCATCATCTGGTCAACGTTGACATTGGTGATATAATTTTT CCAAAATGGTGGACAGCTTTTGTAAAGTTTCAAAGAAAATGAAGTTCACCTTTCCATTGAGTTACAGGATAGTTGTG TTCTTAGAAAACTTTGTGTAGAGAAAAACTGTGCAGAAAATTTTGTGTTTGCAGTAAACAGAATTCTAGTTTAGGGT CTTATGAATTTTAGATTTGAATGTGTTGCAGGACAGTTGAAAGTGACACAGGTTGCAGGACGACACTGTTGTTTGGG ATGGGAGAACATTCTACATTCTTGATTCCCCCCTTTTAAATGTCGATAGCTCCCACAACCATTGTGACAACTAAAAA GTGCTTTCAAATTCTCAATAACCCCCTCTTGAGAAGGACTAGAGAGGATGCCCTCCAGAACATCCTCCCCTGGCCAG ATTGTGATTAGATCAGGAGCTTCCCAGCACCCTCCTGCTCAGAGAGCCTGTGCCACCTCACATGCCACCATGGGAAG CCTGAGATTTTGGTTCCCACTGTTGCCTGGGAATCCATTTCCAAATGAGTGGGGAGAGAGATGTCATCAAAGCTGAG AGTCCTTGGCAATTGATTGTACTGTAGTGCCCTCCACCACCTTTAGGACCTGATTTTGTCTAGCCTAGGTTTGTGTG AATGTGGTAGGGTTTTTTTTTCTGACTTGGTAATAAAGTTTGTAGTATTAGTGACTTGTAGGATATCTAACAAAGAT GAAGTTTTGGAAACTAAGGCCAACTTTGGAAAGTGTTATAGACTTAAAATTGCAGCGTGTGTTTCCCCCTGTTGACT TTGGGAGTACAGCACTGGTATGCTGTTTTTCCAAGGAGAATGAAGTATTGGGAAAGAATTGCTCAGGAAAAATATAA CTAGTAGGATTTTTTCCAGAAATAATAAATGGAATTGAGAAAATTGAAGTGAAACAAGATTCTGTGGTAGATAAATA TCTCTTTTAGTCCCCCAACCCCACTTTTATTTTCTAGTCTATTGATAAACTGTATATTATTATGATAGCCTACTTTG ACTCTTGGTTTGTAAAAATGAAAGTATAATACAGGCTTTATGATTAGGCTAGTGTGAAGTTACATGTTAAAATAGGG GGCTGACAACATATCAAATACCTGTGTAATTTAATATTATGTGATAAATATGTAAAGTAAAAACTGAGATTCCATTT TTTCCAACATTTTGACTTAAGGCTGAATTTTAGGGGTCATTAAATTATCATGGAGCTCAGATAGATACACGGTGGTT CTTTGTCTTACTCTGGGTGTGAGCAAGCTGGTCTTTTCCTTCTTTAAAAATGGTTGAGTTACTTTCTGTCTGCATCT GATACATCCAGTATCTCAAGTCTTTGCTTGCTTATTGTACATTCATCATCTGGTCAGCATGGATATTGGTGACATCA TTTTCCTAAGATGGTAGACAGCTTTTGTAAAGTTTCAAAGGAAATTAAAGTTCAAGAACAAGCTAATAAAGTTTTTT GGCTTGTTCTTGCTCATGGTGTCTTATTTCCATGTTTTCCTGTTTCTTTGATTATACTGGGCATCATATTTGAGAGA TTGCTCATTATTCATGAGAGCAGTGTGAAGCCTGAGGGGTTGTGCTTGTCCCTGGGGTAGTTTGATCCCTTCGGTCA GGTGCTGAGCACACCAACAGGCTTTGTGAATTGCTCAGATGGCCACGAGCTGAGACCTGCAGAGTGGTCTCTCGTCT GCAGTTGCTATTTTTTTTTTTTTTTTTGCAGCATTTCCTCACTAGGCCAGTGGTTTTCAACTGGGGGCAGTTTTGTC CCTCAGGGGAACTTTGTCATGGTAGACATTTGTGGTTGTTACAACTGAGGGGGTGCTGCCGGCATCCAGTGGGTGGA GGCCAGGGATGCCAGTGAACATCCCACAACACATGGGCTAGCCCCACAACAAAGAGGAATTCTCTCTCCTGCAATGT CGGTACTGTCGTGGTTGAGACAGTTCATTGGTCTCTGTAGAACTTGTTTAAAGCCCAGCTTGTCCCTGGCCCCACCC CGGATTTCCCTGCCTACCCTCGGTTCATCCATGGCATCCCATCCTCTTGGGCAGGATTCTCTTGGTGCCATCTTGGA CCCCTTCTTCTGTTGTGTCTTTCCCATCACCCTATGGCAAAAGAGTTTTCCATTTCTGTGGTTTTCCAAGCATCCCA CACCTCCCCTTGCAGCCACATACTTAGGGCTGGTGAAGTAGCCGTTCAGTCATCTCACTGGCTCCAGATTGTTCTCC AGGCATCCAGGGGGCATTTATGCTCATGCCCAGCGCTGGTGGCTGCCTCGATGCTATTGTAGATGTCCAAGTCCTGA GCCCTCGGACTGCTTTCAAAGCCCCAGCTGCCCTACCCTGTCCTCCCATCTCTCGTTCCTATAATCCGCTCTATCCG AAAGAGTCGACTTGCCATTTGTACAACTCCATACTTCACATTTTTCTTTTCTCTGCCTTTGTACACTCTGTTTATTC AACGTACATAGTACAAATACCTTCTTAATCTACCCCCTTTAAACTCCTAACCATTGTTCAAAACTCCTCCTCCTATG AAACTTTTCTTAGTCCTTGGATTTGGATGTTGTCATCTTCAGGGCATCTTCTCTGGGTTTCTTTGAGATCAACACAA TATAGTCAGGGCTGTGGGCCCTGAGCCCATCTGAATAGGATCCCAGTCTTCACTCTGCCACTTAGGGGCCATGTGTC CATCACCAGGTTACTTGAGATCCCCAAACCTCAGTTTCCCTGTGTCTAAAGTGGGGGTTCCAGTGCTATGTTATGAT GCTTTGCCAGGCTACAGTGGAGAGCTAGGTGAAAATGTTCCTGCGACGACCAGTTCCTGTGAGGACCGGGTGAAACA GGACTGATCTAGTGTTGAAAGGTCTCTGTGAGCCGTCAGGCAGGTGACGTTGGAGCCAGCCTCTCCAGGGTGGTGTG CGTGGTGCTGCAGTCTCTGCATTTATCCCATGTCCTTGGGTTTGGCTTCCAGACGGATGTGAATCTCCCCTCTGGAG CATGTAGATGGAGGACAAGGCCTCTGGGGCAGGGGTGGCCTGGAGACATCGCAGTGGTCTTGTCTGGTGGCACACAG ACATTTCCCAGTTTGGTGCGGGCCGCCCTGTGCACAGCTGTCCTACAGCATCAGAGCAGGCTGTGGAGGCAGGGCCA GGCTCACTGGGAGCAATGGCCCTGCTGGAGGAGATCTGGCTTTCAGTCCCTGTTCACAGAGGGTCCTGCTGAATTGG GGACAGGGGTGCCCCAGGAGTGAGCTCTGCAGTATGTAGGAAGTCCTAGTTCCGGTGCTGGGGGAGGAACGTCCTGA GAGTGGGCCGGAGGAGATGGGGATTTATGAGCTGAGCTGTGGGAAGCAGGCTTGCAGAGCCTCTGGTGAGTGGGCCA AAATGACAAATGGATTCTGTGCAACTTTTTATTGTCCAGCCTCACACTGTATTTCTTCGCGCACTGCTGTGTTTGCC AGGGTTTTGATTGTATTAGTTACTTGTGAGATGGAGAAACCTTTCCGTCTCCCAAAATAAATATGGATCTGCTTTCA AAATGTTTTAGCTGTTAAACTTCAGCAGCTAGCAAACAAGCCGAAAGCTTTTGTTCTCATTCCACCACAGGTTTGTA GAGTTTGAATCCCAGATAAAACATGTACCAAGCCACTGGATTCTGGGTGGGATACTAAGTCCTTTGCAGGGAGGTGA CGCCCTTTGTTCTGTGACATTTTTGTTTTTGAGGAGTCTCCCATTTCCTTCTCTCACCAAGTTAGTTTTCTGTCTTG GGTGGGGCTCCCTAATTCTTAGGGACCTCTGGTACAGCCCAGTAGCAGTTCTCAGCCTGTAGGTCCTAGCCCGTAGG TAGACAGATGCTGTGGGGCAGGGCCAGCCCTTTTGTGGAGTAAAAAGCCCTTTTGCGGGTGGTTCTGATGGCCACAC CACTCCAGCCCGCCTGGAAGCCGCCATAGAGATAGCTTTCACATACCTGGTGATACGTGGCAGGTCCTAGAAGGTAT GGGTGAAGGTGGTGTGTACCAGGGGTTGGCAGGGGAAGGAGGAAGAGAGGAGTCGTAGGGCTGGAAAAGGAGAAAAA AGTTGATCTTCACCTAAAACAGTGTGTTTGTCAGTTGAGAAAACTGGTTTTACACTTAGAAGTTAAAGGTGGAGTAT ATCGAGGTAAATGGGTGCTGACCTCTCTGGTATACAACAGATTTAATTTTTCTTTCTTTCTTTTTTTTTTTTTTGTT TTTTGTTTGTTTTTTTATTTCTTTTGAGATGGAGTTTTGCTCTTGTTGCCCAGGCTGGAGTGCAGTGGTGCGATCTT GGCTCACTGCAACCTCTGCCTCCCGGGTTCAAGTGATTCTCCTGCCTCAGCCTCCCAAGTGGCTGGGATTATAGGCG CCTGCCACATGCCCGGCTGACTTTTTGTATTTTTAGTAGAGACGGGGTTTTCACCATGTTGGCCAGGCTGGTCTTGA GCTCCTGATCGCAGGTGATCCACCCGTCTCGGCCTCCCAAAGTGCTGGGATTACAGGCATGAGCCACCAGGCCTGGC CAGATTTAGTTAATTTGGAAAACCCCATTAAATAGCTCAGATCAGATTGCTTACTTGGTTGAAAAAAAGGCAAAGGT AATATTTTCTGTCATGAACAAGGTTTGTGGATATTGCTAATTACTTATTTGAAAGAAATAATAAAATGAAATTCAGT AAAATCAAATAACTTGGCAGTTATTTGAAGGAAGCGGCTCTTTTCTGCTGGGCTGCGACTTTGAGGAGCAGTTGCGT CATTTCCGGGTGAAGCCAAGAGCCCTTGCCATCACCTGGCTCCACCAGGGTTCTCTGTGTGTTTTCCTTTTGGGTTG AAATTTTCCCTTTTAGCTGCATGAGATGTTTATTATTGCTACTCAGAAGTGCCTGGAATCTATATTGCTTTAAGGTG TGGGATGAAGTTTCCTTTCATGATGGTTTAATTTTTATTGCGTTCGCATAGAGCGTTGCCGCCGTTCACAGGATGCC CATGATAGTCCCGCCCCTCGAGACGACATGCAGGATTGCACCTTCCACGCCCAGCACACACCAGGCCCTGCACACGG TTCCTGTGTGCATTAGTGTGGTTGGGCTCCTTCTCTGTGTGTGTGCCTGGCACATTCGGATTTCCTTGTGTCCTTGT GCGATTTCTATAGAAAAAGGTGTGTTTTATCAAGTGGAAAATAGCTTACAGTAATGGTAGTTAATTATTTTGGAGTT CCAAGTTGTTTAGAATTGATCGAATTCCTAAATCTTGAGTTAAAATTCTACTCTAGGATGACTAAGAAAGACATGCT GTGTTTACGTGTATTTTCCAGTTTAACCAGAGGTGACTTTAGACAAGTAATTCTGTTCTGCCAATTTATTTCTCTAA GTATTGTCAATTTAGCTTTTTCATGGGATTCTGGGATACAGAGTTGCTTGGTCCATTGAAATCTATATATGTAATTT TGTTTTTAATCAAACCTAACTTCTGGGTTTTAGAAAGCATCTCCTGTCCACAGCTTTTGAGAAAGGTCCCGTAAATT GGATGACACCAAATGGCTCCAGCACCAGAATGAAAAGGAAGAGCAGCAGAGACTTGGGGCACCCACCTGGGGGCTGT TTCCTAGTGCTGGGCAGAATCCTGGGGTCAGGGTTGGAGGGCCTTAAGGCCAGATTGGAGGTGGGAGCATGGCAAGG TTGCCGTGCCAGGATGCACAGTGTCCATCTGGGGTCCTGGAGCGGCCTGGGTTCTTCCCGCAGGGTGCTGTCCTCTG CCTGACAAGTCCTGTTCCCTGTCTCCTCGGGAGAACACCAGTACCAGGACTCCAGGAGTTGCCAGGTGGGGGCATCT AAGGAAAGGAAATGTTCATGGGATGCGCCACAGAGATGATCCCTCGGAGAATATTTTCCGGTCTTTCATAGAGGCTA AAATATAACATAGTTGTTAGAAATATGTGGTTTTGTAGGAAGTTACATACCTTGGGGAAAACGCTGGAATTGACACC TGAGAATGGGTGAAATGTTGGAAGATGTTGAACAGGTGAGAGGACTGAATCGGTGCAGAGCCACACCTGGTGGTGGG AGCACGTTGAGGGCCTGCGGGTGCCCTTGAGGAAGGGTTTTGGTGGATGACAAATATGATAGGAACCACGTGTCTTG GGGAAAACACTGGAATTGATGAGTGAGAATGGATACATTATTGGCTCTATTGGAAATTATGGCTGACAGATCTGAAG CTCTGTCATGCATTGGGGGCTTGCTGGTTATTTCAGTTTAGTAATTTCATTGATCTGTAACAGATATGTGTCAAAGA GTTTTAGGAAGTTGATAGAAGTGTACATATTGATGAAAATAAAAATTCGTAAGAAATAGATGATGGGTTCAGGTACA GAATAAGGGCAGGAAAATAAATGAAACCAGTATACCAGATGCAGGCATGTGCTTCCCATGATGGGAACCCCAGATAC CAGACGCAGCCAGATGCAGCCACGTGCTTCCTGTGATGGGAACCCGGTATACTAGACGCAGGCACGTGCTTCATGCA GTAGGAACCCCATATATCGGATGCAGCCACATGCTTCCTGTGATGGGAACCCCGTATACCAGACACAGGCACATTCT TCCTGTGATGGGAACCTGGTATACCAGATGCAGCCACGTGCTTTCCGTGATGAGAACCCTGTATACCGTCCGCAGCC AGACACAGACACGTGCTTCCTGTAATGGGAACCCGTATACCAGATGCAGCCTGGTGCTTCCCATGATGGGAACCCCA TATACCAGACTCAGCCACGTGCTTCATGTGATGGGAACCCAGTATACTAGATGCAGGCACGTGCTTCATGTGATGGG AACCCGGTATATTAGATGCAGGCACGTGCTTCATGCAGTAGGAACCCTGTATATCAGATGCAGCCACGTGCTTCCTG TGATGGGAACCCCGTATACCAGACACAGGCACATTCTTCCTGTGATGGGAACCTGGTATACCAGATGCAGCCACGTG CTTTCTGTGATGAGAACCCCGTATACCATCTGCAGCCAGACACAGACATGTGCTTCCTGTAATGGGAACCCGTATAC CAGATGCAGCCTGGTGCTTCCCATGATGAGAACCCCATATACCAGACGCAGCCACATGCTTCATGTGATGGGAACCC CGTATACCAGACGCAGCCACGTGCTTCTTGTAATGGGAAACATCTTTGAATATGCCAGGAATTAAGCAGTGAGCTTC TTGATGGTCAGAACACTGATGGCACCCAAAGTAGTTGCGTTAAGCCAGCACCAAGGTTCTGGGTTGTGTGTTTTGTT TAACACAGCCATATTCACCTGGCCATCAGTTTTTTAGTGAGGAGACCTTCCCAGGGCTATTGGGGATCAGATGAGGG GCCTAGCATATACTAAGGGCAGAGCTCTGAATAGGTTAACAAGAGCCCTAGATCCCATTAGAGCTGGGACTAGTTTT ATCTGTGCAGGCCTAGAGCCCCCCTATGCAAAGTGGCGTCTGGGCTGGCAGCATCACCTGGGCCTACCCACTCCTGC TGTATCGGAATCCGCATTGCACAAGCTCCCAGCACTGGCCGCTGTGGAGGGAATGGCGCTGTCTCGTGGTACCTTCT CCCCAAGGCTGGCTGCTGCTCTTCCTTAGGTCTGAGTCTGGCTGTCCTCTTTCCAGGTCGAGGATTTAAAGCCGCCT AGCTCAGGGCTCAGTCCTGGGTGTGTGGCTGGCCAGGAAAGGGCAGTGAATTCAATTTGCTAAGTGTCTGCTCTGCA GATGCAGGATGCTGGAGATGGGGGGCGCAGAAAGAAACAGGCCCTGATTCTGCCCTCGCACGTGGTGGGGTAGAGGC GGCGAGGTGTTGGCAGCCATCCCTGCTTTAATTGACCTGAGTTGTCATTCCCTCAGCGATCAGTTTCACCTGTTTGC GGACTGCCTGTAGTACTGCTTGCTTCTTGTTTTCTTTATATCAACTTATTTTTAAACACCCGCTTTAACTTCTTCCT AAGGAATTGCAGTGAGAACAAGTGTTTGCTATGCTCATTTTGTTTATTCTGTTACACGTTAATTTAAATCTATAGTT AATAAACTAGTGTCTCAACTAATCTCACCACCTGAGGCGTGTTCAACACCCTAGAGTCAGACCACAGCCTCCCTGTG ACTTCCCCACACCAGGTCTCTGGAAGGGTCGTTCTCATGCCTTTGAGCTTCGGGTCTTTCATCATCAGAGGAATGGA CTGTGTCTCATTTGCAGGGCTCTTGTAAGGATTAGAGAAGAGGAAAGTGAGTGCCTGGCATGAAGGAAGTACCATCA ATGTCACTAAACAACTCTTACTATTAAGTGTTTCTGAGTTGAGTTGTGGCTTAGCGATGCGTGAAGCAGTTGTTGAA TTGTAGTCTGTGCCGTTCAGTGCCTTGAATATAAGTATATTTGATAACGTTTATGATAAGTGTTGGTGAGGATGTGG AGAAATTGGAACCCTGTGTGTTGCTGGTGGGAATGTGAAATGGAGCAGCTGCTGTGGGACACTTCTGGAGGTTCCTG AAAACATGAAGCATAGAATTCCCATGTGGCCCAGCAATTCTACCCCTAAGGACATACCCCCCAAAATTGAAAGCAGG GTCTTGAGTAGATATTTGTACACTGGTGTTCATAGCAGCATCATTCACAGTGGCCAAAAGGAGAACACACACAAACG TCTATCTGTGAGTGAACAGATACATTAAATGTGGCCTATCCATGCAATGGAACATTATTCAGCCTTAGAAAGGGTGG GAATTCTGGCACATGCTGCAACATGGACAAACTTTAAAGACATTACGCTGAGTGATATAAGCCAGTCACAAAAGGAC AAGTACTATATGGGTCCACAGACATGAGGCACCCAGAACAGGCAGATTCAGAGATCAAGTAGAATAGTGGGTGCTGG GGGGTGGGGAGAGCTATTGTTAAATGGGGACATAGCTTTTGCTGAGGTTAATGAGAAAATTTTGGCTATAGATAGAG GTGATGGATACACAACTTTGTGAATGTATTTAATGCCACTGAATTACACACTTACAAAATAGTTAACATAAATATTG CATTTTGCCACAATAAAAATATATTTCCTAACATTTAAACAAAAGGAGTGTCTGGGACGTTACCTCTGACACACCTG GTTATTTGGCACCACCGGCGACCCCTGGCTGCTGGCAGGTGTCTGGAGCAGCAGCTCAGAGCAGGAGGCCCAGAATC CACAGTGCCAGGTCTAGGAGTGCAGGGGTGTGGCAGGAAACGTGACCCCGCGGCTCCTCTTCAGAGCCTCGGGGACC ACCAAGGCAGTTCTTGACACTTTATCCTGGCCTTGGTGTTCAGCATCGGAAAACAAGGAGAACTAGAATGTCTTGAC TTTCTAAAGGTTGTTGTAAAACAAGGGAAGGCCATGATGCAGTGAGTTTTATCACACTCTTGCTGTGACCTTCATGA GGATGTTGGAGCGTCTTTTCTTCATTGCGGACAGAGGCTTCCAAACTCCACGGCTGAGCCCCAGTTACAGTCAGTGT GCAGTAAGTCCTGTGGGTAGTCGTAGAAGTGGAGAAAATGGCAGTATCTTTTCTCTAAAAAACGTTTACAAACAGAT ACTTAATGTGAGTTTGCTTGCTAATAGGTTAGGTTCTTAGAGTTTAAAGAAGTAGTTAATTATCAAAGCTTTTTCAT TCTTGACAACCAGCATTTTTCCTCTAATTCAGGCTACCTTTGAAATGAACACTGCAGATCTCTGAGATTTTCATCTG TGAATGTTGTTTGAAGAATTCATTCTGCTGCAATTTAGATAAAATGTACCTGCTGAATTTTGTAATAACATTCACAA AGTTTTATATTTTTGTTCGTGTTTGAAATATACAATAGAGGTTTTCCTCGTGATAGTGAGAATATATCAATATGAGA GAGATACAATTATATCAATCAATTATTTGAACAAAGATTGGGTATTGTTTACATATAATTTTGTAATAATTCCTATT TCCAAGGAAGAACAATGCACAATACTTTGTCATTGTGTCATTTATTTTTATTGTAGTCTTTTGTTTTTATTGAAACA TAGATTCCATGTATAAATGTGGATTTTATGTATATCTTTCCCCGTGTATATTAGAAGTATTTCAAATCAAATAATTT GGCCAAATGATTTTTCTATATTAAACATTATATTTTCATTTAAACCACTATAATTTCTCCTAGTTCAGTCTGATTCC TTCTTGCTTTTTTGCATACAGTGAGTACTATGGTCCAAATGTTTGTTCTCCCACAAAATTCATGTTGAAACCTCGCC CGTAAGTTGTTGGTATTAAGAGTTGGGGCCTTTGGGGGGAGATTAGGTTAGTGCCCTTATGAAAGAGACTCCAAAGA GGTAGAGCCCTCGACGATTGAGGATGCAGCAGGAAGGCGCCATCTGCGAGGAAGTGGGGCCTCACCAGGCACTGAAT CTGCCGGCGCCTTGGTCTTAGACTTCCTGGCCTCCACAACTGTGAGAAAGAAATGTCTGTTGTTTATGAGCCACGCA GTCTATAGGATTTTGTTATGGCAGCTCAGATGGCCTAACACAGTGAGCATGCCCCACCCGGAGATGGCCTTGCCCCT CTTGCTGTCAGCATCAGGACTGACAGAAGCTGGGCCCATGCAGATTTTCCGGAAGCTGTGTCAGGTATTGGGGGTCA TTGGACTCTAACCAGTACCCAGCAGACAGATGGGACCTGAGAGAAAGGGTTGGGGTCTGCCCCTTCTCCGCTCCTCA GCTTCCTGCAGTGGCTGGGGGCAGGAAGGAGGGCACAGTTCAGGGGATTGAGAGACGTGTGCTGACTGTCCAGCAGC AGCAGATCCACCTGCAGGCACAGTGCAGCCTGGTGTCTCGTCTGAGAGGACACGTGTGCAGGACAGGGGGACCCAC AGCTGCTGCTTCCCCACTCGCCAAGGCCCCGTCAGGAGCAGCAGCTGCGGCCCTGGGAGCCCACAGTCCAGCCAGGA GGTGGTGAGCACAGGGCGGAGTGTGGGGTTGGGGACTCCACTTGTGGTGTTCCCAGATTTGGGGGATTTCCGCAAGG CCCCGTTGAAGGGCAGTGCTCCTTAGATACAGTGCAGAGTCCTTGGGGTGAGGGTCCTGTCTCATTACTTGATAGCA CGTGTCCTGTCTCATTACTTGATAGCACATGTTTCAGCTGGCTCTTTAATGGGAAAGTTGGTGCTTTAGTGGAAGCT CCAGTCCCCTAGATACAGAGTGTATTGCGGATATTTATCCCTTCCTGGCTGAGCGCGGGGCAGTTTCTGCCTGCAGA TTTGGGCTCTGGCTCTGGGTTATGGACCTTTGAATGTGGCCATGCCAGAGTTTGTGACCTTGACGTCTTGAGGCATC CTGTAAGTGGTATGACCTCAAGGGAAGAAAGCTAGCGTGGCCTGCATACAGGTGGGCCTGTGGACTGTAAAAGCAGG TTTACTTAGCGGGAGTAATTACCCATGTTGATGACAGCCTCTAACAGAGGTGCAGTCTTCAAATTCACATTTAAATG TCTCTGCCTGCTACTGAGAAATCCACAGCAAGGGAAAAGAGCAAAACAGTCTTTCTGTGAAGGGTCTGTTTTATTTT TTATTTTATTTTATTTTAATTAATGAGACGGTGATCATCCAGGGAACAGGATCTAAACGCAGTCTTTGAACTTGAAC TCTGTATGTGGTGTCTACAGGTTTGGGTGTGAGCAGCCTCACTCTGTGCTGGTTCCCAGCTTAAACGTCCAAGGAAG AGCTCATGACAGTTTTTATATTAGTTCTTTAAATATTTTATTGTAAGTTCGTCTCTTCCCAGCCCCTCTTGCTATTT TTATTGCTCCATTTGAAGTTCCAGCCAATAGCCCAGGCCCTTTCTGTCTTTGATGTCTGTATGGTGGGCAAGATCTC ATCACGACCCACATCAGCTTTGCTGTTAGAAACGTGTTGCCACAGAGGAGCACATTCATAATTGAAATCACCAAATA GGGAATTCTTTCTAGAGGCAGTGGCTGCCTCAGTGAAACGTGCATGGCTGAGCAGAGCAAGGAAGAGTTAGTGGGGC CGGGGGCGTGCTGATATCACAGTGGCGTGGGGGGGGTGAAGGGATACAGGTGTGTCCCTCCTCTTACCCGGCATGG TGTTTCCAGAAAGCAGATGGGAGAGCTGTGTGATGAACAGGTGAGACACTGGCTGGACCCAGGTGAGCTGCTATAGA AGCAGAATCAAAACCGTGTAGCACGTTGCCTTCCCTTTCCCAATACTGTAAAGAATTCCTCTTCAATGGAAATCTAC TTCAAACTTTTATCAGATTAATTTTAATAAAAAGCTGAATCAATACGAAGCAGCTTGGGAAAACAGCTTTCCCCCAC CCCAGCCCCCACCACCAATAACATTTTACAGATTCAGGCCCAGTATTTCTTGTTTCTAATGAGATGGAAGGAGCGGT ATTCTTGGCACCTGCTCATCTCCTTCCCTTTGGAAACTCAGCCAGGTTTTTGTCGTTAGCTTATGGGTCGCTAATGT TATGTGCACTCATAGCAGAAATGGAAATCCGCCATCGGTGAAAGTGGAGACCTTTGAGAATTTATGCAGCATCAAAA TGTAAATACCAACTCTTATAGAATATTGGGGTTAGCAATGTCGACAGTTCTACCCCGAGTGCGGTTGGTGGTGGGGC TCCAATGGAAACAAACTGGTGTCCAGCAGTCAGCTGTCTGCGCATCCGTCCCTCCCACTCCAGCTTCTCCAGGTAAG ACTGGCCATGTGAGCACAGAGGGTAGAAGAAGGCCTTAGGGACACCTTAGAATGGGAGGAGAGGTCATAGCTGGAGC TCCGCTTGGGGGTTTCTGGTGCTCCGCCCATGGCTCCTGGACAGCGAGGGGCAGTCTTCTGAATGGACCTCAGAGGT CCTGTGGTCCTCTTTGGCCAGAGGGCACGGGCCCCCTTAGCTCATTTCTAGTAATGGGATGGGTGGGTGTTAGGAGG AAACACGCAGCCCAGCACAGGCTCCCCAGAGCACATCGTGAATCTTTCTGTCGCCTCCCTGCCCCACCTCACCTCTG TTCTGTGGCCTCCCTGCACTGTGCCCCTCCTGTGTCCTCAGTTGCCACATGACTGACACGTGCTTGGGGCTCATTTG ATGGCTCTGGCAACTTTCTCTACCTCGGTTTACCTTTTGGCTGCCCAGTGCTGGATCGCCAGTCTGCTTTACTGTTC ACGGGAGGAAGGGTGTCACGGCGTCACACATGGGCATGGCACCTGCGCTGGGGCTCACCTGTCTCATGAGGCCGTAT GTCGCACAAGGCTTCTTGAGGGTGGGGTGGCCATGAGCGAGAGCGCTCGCCTCTGTCCTTTTCCTGTTGCAGAGGAG GAAAAAGTCTGGGGCCCTGTTTGCTCAGGGTGGGCTGAGGCCCAGACCAGCTTCCTGTCTCCACACCCTGACTCCAA ACCCTCACTGTGACCCCACGGGAAAGCATATTCCCAAATGTAACTGCGACGCCGCAGCCCGGGACTGCTGATTGAAT GAATGAGTATTCTCCTTTGGGTTTCCCACAGAAGTTTTCCCCCAGTTCCCTACCATGATGGGTTGTGGCTTTGTCAC CCGCAGTGAGAATATGCGAATGCTTTAGCAGATGTTGTGTGGCTCGGGCCTGCCTGCCTTATTTGGAGATATTTCGC ACCTCTGTGGGCTCCCAGTGGCAGGCATCTCTGGGGAGGGAGCGCTGTCTCCATCGTCCTTTGGGAGGATGTGGGAT GGCTGCACCGGGCACCACTCTCACATGGTCTCCACATCAGGAAGCGTATCTCACCGTGTCCAGCATTGGTGTCCTGG CCAGATGCTCTGTTTATAATGATAGAAAAAAAATTTTTCATCTCTGAAATTTGCCACCTTCTTTCAGCAGCATTTCA CTTGGTTTACTTCCTTAATTCACGAAATATGTGAAAAAACTTTATAAATCTTAAAGATGAGAAAATTCTAAATGATG TGTGTACAGATACACACACATACACACACACACACACACACACACACACACACACACATTTGGCCCTTGGACAAGAC AGGTCTGAACTGTGTGGATCCACTTACACTTAGATGTTCTTCTACCTCAGCCACCGCAGAGACAGCAAGACTGCCCC TGCCCCTCCTCCCCTCCCTTCCCCCTGCCCCTCCTCCCCTCCCTTCCCCCTGCCCCTCCTCCCCTCCCTTCCCCCTC CCCCTCCTCCCCTCCCTTCCCCCTCCCCCTCCCCAGCCTACTCACCATGGAGATGATGAAGACCTTCATGATAATCC ACTTCCACTTAATGAATAGTAAATAGATTTTCACGTCCTTAGGATTGTCTTAATAACTTTTTCTAGTTTATTTTACT GTAAGAATACAGTGTATAATATGGGTAACATACACAATATGTGTTAGTTGAATGTTTATGTTATCATTAAGACTTCC ATTCAACAGTAGGGTATTAGCAGTTAAGTTTTGGGGGAGTCAAAAGGTTATGTGGATTTTTGACTGTGTGGGGCTTC AACAGCCCAACCCCTGCACTGTCCGAGCATCAGCTGTATACATACATATTTTGCCGTTTGTATTATAGTAGCTCTGT GCATGGGGCATGTCTCCGCAGGAAGAGGGACTGTCCTGACTCTTGGGCCTGGTGGTGATTGTCTGGGGCACTCCTCC CAGAGCCCTGACTACTGACAGGTCACTTCAAGAATCAAAGAACCTCGACTATGATGTCATTTTTCTGTAAGCCCTGG ACTACTATAAATTATGTTTTATCTGTCATAATTAAGACTCGGAGACTACCAAGCTGTCTTCGAGGGGAAAGGCCTGT CCCTTTCTCCAGAGAAATAATTAATTGTTAGAGTATATGGTTTTTGAAGAATTTACTGTAAGATAAAAGACTGGTAT TTTATTCTTGCATTTATTAGTATTTAATCAAATGGAAAGTGCACTTTCAGCCTGGAAATCTTGGCTTCAGCTGCAGG ATTAGACTGAATTATTGTTTAAAGAGTAAAAGGTATAGAATTATATGTTCAAGTTATCAAGCCACTGATAGGTTTTG GGTATAATGTAACTAATTTAGAACATAAAATTTCCTGCCTTGTGGACTAGAGCATCGGATTAATCATTTGGAAGCGG GGCGCACTTCCTCTGCTGCGTGGCCTTGCGGTGAGATGGAGCCTGTCGCTCCAGGAGCACGAGACCTTTACGGAGTT CAGTGCCGTCAAGGCCCACTTGTCCACAGCCACCGGAGCATGTTGGGAACACCGGGAGCCGCTGGAACGTGTCTGGA ACACCAGGCTCTTCCATTCGGGACACTGAGTCATAAATCATCCCATGTGTCTCATGCAAGAATTCTTCCCTGCAGAA ATTTCATGAAACTTAGTCTTTGCTAAGGGTAAAGAGGATTTAATTCTCTTTGTTCTCAGTGGTGTGCAGGCTTAATG CTCATGGGTGTTGACTGGTGGCCGGCACATCAGTGGCTCGTGGGTGTTGACTTGTGGCTCCTGGCATTGCTTTGCAT TAGAGGATAGCCGGCCTCTCTACACCATGACTTAGAAAATTCCAGCCAGGCACAGTGGCTTACACCTGTAATCTCAG TGCTATGGGAGGATTGCGTGAAGCCAGAAGTTTGAGTTCAGCCTGGGCAACATAGCAAGACCCTATCTCTACCAAAA AAGAAAAAGAATAAAGAAAATTCCAGAAAACGTTTGTGACAGCATCACTGTTGAGGTTGTAGTGCCACAGGCAGTGC AGCAAGCATCTGGTTTATCTGCGTGAGCTCATGACGTGTTCATCAGTGGGCAGAAAAAGATGGGATCAGACAAAACA GATGCTTTCAGGATCCTTCAGATCAGGATGGCAAATGTTTCCTGAAATGGGCCAGGCCAGAAATATTTCTGGCTCTG TGATGTACAGCCGCTGGCACAGCTACCTGACCCTGCTGTTAGAGCTGCTGCAGACAGGACCCAGAGGAGGAGTGGGG CTGCTACCCATGATGCTAGACCGAGGTTGCCGAAACTGGAGTGTCCCATTGTATGCACGTGTCCTAAAGCATCTCCT TCCATCTCCTTGTCTACTCCTACCGTGCGCATACAGAGCCCCTATGGTGCGAGTACAGAGCCCCTGTGGTGCATGTA CAGAGCCCCTATGGTGCAGGTGCAGAGCCCCTACGGTGCAGGTGCAGAGCCTCTACAGTGCATGTACAGAGCCCCTA CCGTGCAGGTGCAGAGCCTCTATGGTGCGGTGCAGAGCCCCTATGGTATGCTTTCAGAGCCCCTAAGGTGCACCTAC AGAACCCCTAAAGTGCAGGTGCAGAATCCCTACAGTGTATGTGCAGAGCCCCTACGGTGCGGGTACAGAACCCCTAC GGTGAGGGTGCAGACCCCCTATGGTGCGGGTACAGAACCCCTACGGTGAGGGTGCAGAACCCCTAAGGTGTGCCTGC AGAGCCCCTATGGTGCGGGTGCAGAGCCCCTACGGTGCGGGTACAGAACCCCTATGGTGCATGTACAGACCCCATAT GGTGCGGGTACAGAGCCCCTACAGTGCGGGTGCAGAGCCCCTATGGTGTGCATACAGAACCCCTACGGTGCGGGTAC ACAGCCCCTAAGGTGTGCCTACAGAGCCCCTACGGTGAGGGTGCAGAGCCCCTACGGTGCGCGTACGGGGCCCCTAC GGCACGCAGGCTTGCCCCGCCACCTGCTGTCCCCGGATTCCTGTCAGATGTCCGGGTGCCCAGCCTGACTGGGCATG GCCCTCCTGGAAGCAGTTTCAGGGCCAGTTCTGATCTCAGGTGGGGATGGAGAGCTGGTGTCTGGGCCTCTCAGAGC TCAGGCCCCGCACATCGTCAGAGTGACCTCACCAGCTCCAAGTGCCCCTTTTCCCTCAGTTTCATTATTTGGGAAGT GAGGAGGTCACACCAGGCTGGACACGGGATTTCAATCCGTGTGTCCCCCTGTTGCCCGGGGTGGCTGCATGCAGGGT GAGTTCAGGGAAGCTCTGGATGCAGTGAGCGAGGCCCCGCCCAGCCTCTTGGTGGGAGTCACTGGCCTCACAGCTCA CTGCAATTCAGAGACCTGCAGTGTGAAGATTCTTCCAGATTACTGGATGGAAGGGACTTCAGTGACCTCATGTAAGA AAGGAAGTCACCCACTGGTAGCCTCAGAGAGGGAACCTCTGGCAGTCTGGCTCACAGCCGGGGCTAGATTCAAACTG TATCCAAGAGCAGCTGCTGAGAGACCCACCTCAGTCCTGCAGGAGTCCAGGCTCTCTCCCAGGCCCCCAGGGAGGGT GTTTCCACCTGGTACCTTGGGGAGATGTGACCAGAGCTGCTGGAGGGAGCGTCTACCTGCTGGGGTCAGGGGGTCTC CATCCGCCCACAGTGTCGGCTCAAGAAGTGTGTGCTGTCCACCCCAGGCCCAGGGCAAGTCCCACGAGGCCGAGGCT GCACCTTGCTGTCCAGGGGGCCGCAGCTGAGGCCTGTGCAGCCTCCCCGGTGCTAGTGCCAGCATGGTACAGGAGTC GCAGCTCCAGGAGGACATGCAGGCTGCCTGCCATGAGAACATCCCTCTGCTGTTTGCAGACCGAGGCCCATGGGTGC AGAAGGAAGCGTGCCGGTGCAGGGCACGCAGGGCCGATGGGAGATGGACTGACCAGTGGTCTGGACCGAGCAGGCAG CAAAGGGCCCTGTGTGCTGAACTGAGCTGCTGGGACTTAATGCACACAGAACCACAGAACCACAGAACGTAATGGAG AAGAATTGGGTAAGGGTGAGTGGGAGATAAAGAAGCCATCAGAATGCTGCTGAGTCTGGTGGGGGTTGGAGGTCACC GGAAATAGCTCCTCCCAAGTTTTACTCAGATCTTCTTGGCAGGGTGCATCTTAGCATTTGTAGAATGCACCAAGGAA TGCCTGGGAAGGGCATGGGGGCCCTGCAGGGGTCACAGGCTGGGAGAGGAGGGGCTTACTGCCTTGGGCCCCTGGAC ACAGGTAATGCCTTTGCCTCAGGTGGCCAGAAGTGCCTGTAGGCTTTGCAGAAAGCTACTGTTCCTTTCCTGTGTGG GTGTGTGACTCCTGGACGGTTGCCGGAGATTGCCCTGAGGCTGGGGTGGGTGCCCGTCCTGGAGCCCCCATCCCACA GTGCTCTTTGTCCGTAGGATCTGCGCACACAAGGGTTTGGTTGTTGAACCTGGAGACACACATTATGGTGAATCCCA GTGTTGACTGATTATATCATTAAGTGGGTCACATGACACAAAAACCACACGTTCTGAATGTACAAATTCCATCCCAG TGCAGGTGGAAATTTGTATTAACCATGGCTTTCTTCACATGGCTTCTTTGCAAACAAACACTTGGTACTTTGCTAGT ACAAGGTCCTTCCTTGGTGGGGAGGGGTGTAATCCCATGTCGATGTCTGGACGAGGAGAAAGAGGAACTTCCCCTCA GCCAGTGGACGAGACTGTCCAGGCTGCTCCACAGTGGCCACAGAGAGCCCCCGGCACCTGCTCGGGGCAGGAATTGG TGGCTTTGGCCACCTTGCTTAATTGAGAAAGTTGATGTTTCCGTCAGATCTTCTTGTGTTCGCACATGTTACCTATT CTCTAAGAAGTCAGTGAAACTTCACCCAACTTTAGAGGCTATAAAGAATGAGGTTATTTTCTGCCATGACATATTTA AGCTGAAGCTTCCCAAGCCATAACAGTTTTAAGAAGTGACATTTCATTTTGAACACCAGCCTGCCACCTCTTTACTC CTCATGACAGACTGTCACTGTGCAGCGCAGCAGTTAGTTGGTTCCTGAAGTGCCAGAAAATTCCCAGGTTGTGAGGA GTGTGCTGCTGGTGGGGTGAGATGTGCGACCCGGCGGGGGCCTCGGCGGGCACAGGCGGGAGACCCGGGCCAGAGCC CTGTGGGGTGAGCGCCATCCTGGCCAGGCCACACGGCCGGGGCAGCTTCTTTCCCTTCTGCAAGGCAGTTGGCATTA TCGCGGATGTTAGCATAGGAAGTTCTAGCGGGACTAGTTAAAAATGAGGGAAAACATGGTGTTCTCTCATTGTCTTT TTCCTAGTAAACTAAAGGAATCATGCTTGATGAAGGCTCAAGTGTAGTATGATTCAATCGGGCATGAGACTGTGCTG AGGTCTCGGATGTCTCGGGCCTGGGGTCAGCTTGAAGAGATTCATAGGCGGGTCTCGAAGCCCATGCTGCTGGGAGG GCCGGGCTGCCGACGTGGACGGGTACAGCTCCCTGGTTCACACAAGACTTGGGGTTCCCTCCACCGTGGAATCCGTC TTGCTTTCACGGGATTTGGGGACTGTGAAATCTCTTCTCTTCTAATGCATTCCCGTTGTCTAGAGGCTGGGGAGCAC CATTTGATTTTAAAGGACAGCCACAGTCCTGCCAGCTCCATAGAGAGAGCAGAGGTGGCCTCATGTTCTGAGCAGAG GGTGCCATGGTATTTCTGACCAGGCGGGACCCTGGCTGGACTCACGAGATTGTGTGAGTCCACAATGTGCTTGTCAG AAGCCAGGCGTCCTGGAGTTAGGGTCAGATTCCCATGGTGGGTTTCATTACCCTCCTAGAGACTGGCGGGGTCCCAG CCTCTTGGAAGTGTCCGCTCAGACACTGCCAGGTGCCTTGGAGGTAGCAGAAGCATCTCTTGATGGCAAGTGTCCAT TGTGACCTGTGTTGAAGACAAGGTCTGTGTGAGAAGGTGCACTGGTACCTACTGTGTCCTGATGACCTGGGAGTGTC GTGAAGACAGAGCAGCCAGTGACCTTCTGAAACCAAAAGAGTGGCTGCCCACTCTGCCTCCTGGCTCTGGCACGGGG ACGGCTCCAGGGTTCTAAGGCCTAAGCTGGCTGCACTACCTCCCGCCCTCAAGGACGATTGGCATCTCTGGCGTGCT CAGGCCCCTGCAGCCTTGCCAAACAAACAAACAAACAAAAACAACTTGTAATTTTTCATTTTATCTGTATTTTGCCA TTAAAATGTTGTTCTCTACACAATTAGCTGCAGGACGCACAGTGTTTAGCCTGTGGACGTGTCTCCCAGCTCTGCCA TGCACCGTGCGTGTACACCTGAGTGTACTCAGGTTTGCATACCCCCGACACTGGGACATTGATGTCATGGAATCTCT TCATATAGATTTTTCTTTGGTCTGCAGTGACATGGCAAGTGGTAAAGCCTTTTATGAAACCTCTTAAAGAAACAGAT GATAGTTACTAAGAAGCTTCCAAAAAGACTTTTTTTTTTTTTTTTTTTTTTTTTTTGAGACAGAGTCTCGCTCTGTC GCCCAGGCTGGAGTGCAGTGGCGTGATCTCAGCTCACTGCAACCTCCGCCTCCCGGGTTCACGCCATTCTCCTGCCT CAGCCTCCCGAGTAGCTGGGACTACAGACGCCTGCCACCATGCCCGGCTTATTTTTTTATTTTTAGTAGAGACGGGG TTTCACCGTGTTAGCCAGGATGGTCTGGATCTCCTGACCTCGTGATCCACACGCCTCGGCCTCCCAAAGTGCTGGGA TTACAGTTGTGAGCCACCGCACCCGGCCCCAGAAAGACGTTTATAGGCTCATCTTGACAGTGTTCTGGTATCACAAA CCAATTGTCAGTTAGAGCTAGAGTTGGTTTAGCTAAACTGTCGGAATGGATATGGGAGTGGCAGTTATAATCTGGAA ACATTGTTCCAAAGTGCTTTAAGTTCCAGGGAATTTTTGGAGCAATTTGTTAAAACTGTACACATTTACGTTTTCTG CCCTTTTCTCTAGGTGTGATCTATTTCTTTCACACACACACACAGCAGTTTGTATGTTAACTGCTTGCTGACTGATT TCCTGCTTGATTGCACAGATTCAAAGCAGTGCATGAGCTTCAGAACAGCGTTTATCTTCCTGCATTACTCATATTTA GTTCCATATATGTACCAAAGAGAAGAGTTCAGAAAAGAAAACCAGATTTTATTTTAAGTATGATTTTTAAATGTAGA CAGGTGCCAGTTTATATCTCAAAATGGGTGATTTAAAGACCAGTGAACAGATTTCCTTAGATGTCCGTCTGCTTCTG GGATCTGATGTGTAGGCATCTTCACGTTGGTTTTATAATGTGCCACGCGTCAGGTGTTATTTCGATGACTATGCTGA GCCCTTTAATAAGACGCACGTGCCTGGGGTGCTTCTGGAACCTTGCCACCAGGGCTCATTCTTTGACCATCCCTAAT TCCCCAGGGCTCTTGTTGTTAGAGTATTCTTTACAACCAAAGCATCATTAATCAGAAAGGTGCTGTGCGGTGCGGGG CCGGCTCCGATGACAGTAATAAAAACCGGCACACCTCCTGTTCCAGGACCCAGACCCTGGAAGTCTGTGCCTAAGCC AAGACATGAGCAGCGTTTGTGGAATGCGGCTTCAGATAGAAACCTTCTTTCAATTTGCAGGCCTGGTTATGTAATTC TAATGCAGAATCTCCCCCTTCGTGTTTGCATGCGTGTAGCCGGGTTCACAGAACTTTAGCCACAAACAGGAGTGTTT CCCTTTTTCCAGGGCTCCCGTGTTAGGCCCCCACCAGGAGGCTTTGGGGTGGCGTTTGGGAACGCCGTTTGGATCCC TTCTCAGTTTGACATCCTAGGTGGCAGCTCCAGTTTTGTTTGGTGAAACGATTTCAATGTAACAGAACCCCTTTCGG TTGTGAGAGCAGAATGAACTGAGCCGCACTTGACTGTCTTGTGCTGGATGTGACCCAGCCGAATCCACGGGGAGACC CCTGGTTACAGCGGGGACCACGGGTGGGGTCTCAGCCGTGGCCCTTTATACTTGGTGAGCATCAGCCACACCACGTG GCCAGGTTTATGATCAGGTGATAGGGATGAAGTGTTTTTGACACATGCTTTTCTTTTTTTAAATTAAAATACTATCT CATGCCTGATTTTGAGGTGGGCTGAGCATGAGTCCTGAGGATGAGAGGGGCTTCCCCAATGCCCCTTATTCCCTGTT CAGAAGGGAGATTGCAAAGCTAGTTTTCATCTTGTGATGTCTTTGTGGGAGGTTGTGAGGCAGTCAGACACGGTCCT CTTCCTCCCCCAGGTGAGCCAATCTCCCCCTTCACACAGACCCGAAGCCCAGGCCACAAGGGACCTGGGCTACCTTG TTTGTAAACAGCTTACAAGGAGGCTGGTGATTTCTCCCTTTTAACATGTTCGTTTTTATGTTTTTGAGGTGTGGTCT CACTGTGCTGCCCAGGCTGGTCTGCAACTCCAGGACTCAGGCGATTCTCCCACCTCCGCCTCTGGATAGGTCGGACC GCAGGTGCACGTCACCACACCCGGCTAGTCTGTTTCTGTGTGTTTTCATTGCACAGGATACCAGCAGTGGAAATGGT TAAGAGGGAAAGACACAGAGCTGCAAAGATGGTCCTGGAAGGACGGTTTTTAGACCTGACAGTTTTTAGTGCAGCAG GCCTGGAAAACGACTACCAGTAGGGGAAAAAGAAGAGTTTCTGTTTCTGGGTCTCTTCCTTTTAATATGGCAGGAAC TGGCCTCTGGCGCCAACTGCTTAATTCAGAACATTGTTATTTAAACGCCATTCATGTTAAAAGGGAGATGAAACGAA ATTCATTTTTCCAGAGGTCTCCAAATAGCCATGAAAAATAAATTTTTTTCTGCCACCAAGTACCATCAGCCACTCAT GTGGTTTAGGTGGATGCCGTGGTGGTGCCGGGAGCACTACCAGCCTGTGGGGCCGTGGGCTTGTGCTTCGCCGGAGT GGGAAGTGGGGACATTAGGAAGCCCGACTGTTGAATCGCCTGGGCGTCCTAAATGTGGGCTGTGTTGTCGTCCCTGT GCAAGCCTGAGAGGCAGACAGCACTGAGTCCACTGGATGCCTGCTTGTCTGGTTGCCCCTTAGACTATGGTACTCTC AGCAACACGGCTGTCCCATTATGTGCACATGCCCTGTTACAGCTTTTGGAGCCCCCCTTAGGACCGATGGTTCGAAT TCTGGAAGTCCTAAACATTTGGGTCTTCCACGTCGCTGTTTATTCTGCAGATTTGAGGCTGGCGTGTTTGAGTTAGA CGGGACACTGGGGCTGCCCGCTTTCCTTTGATGGTACGCCGAGGGCCTCTCTGCCCCGTGTGTTCCCTGAGGTTCCA GTGGGCTGCTTCAGGGAAGGCGGGCCGGGAATTCCTCTCTGTAAGACAGGCCCCGTTTTATGCAGCTGTGAACCTTG TGCTTGGGTGTAGTAGGTGGTCGGCTCTTTTCTTTTCTCCTGTTCACAGCACGCTTATAGCCAGAGCAGACTCTATG GTTTGCCTGGAGAGAGCTTGCACACACATCGTCACCCTGTCTCTGGTGGTTGTGGCTGCAGCCTCCCAGACGCCTCT CCCTGTCCCCACCCTTGACACGCCTGGAATCCGTGTTCTCCCTCGTTTCTCCGACTTCATCCCAGGACTTGGCCCTC ACGGCTCCAGGAGCTGCTGAGGCAGCTTCATCTCAAAGCCAGGTCCTCTCCCTCCTGGTGACCTGGCCACCACCTTC TGCTCCTTAATGCTCTGTGCTGCTTCCAGCGCCAGTGCCTTTGCCTGTGCGCACCTGCCTGCCTCAGTCTCTCCTCT CCTCCTCCTCTCCACCCAGAAAACTACAACACTATCCCTTCTCTCTCAGCACCTCCTCCAGGAAGCCTTCCTGGACT CCCCAGGACATCCAACCCCTTGATACAGGCTCTCATTAATCCTGGAGTTCTCCTTCTGAATTCCTATCTCAATTATC ATTGCATGATTCATATAATTAGATATTTTGTTCAACATATATTTGCTGAGAAATCGTGGGTGCCAGGCACTGTTATT CTAAATGCTGGGGATACAGCAGTAGATCAGCTAAGACTACTCTGGTTTGAAGAAAAAACAGGAATTCCTCAGAACAC AGACTTGAAACCAGCCCAACGTTCCTGTTCTCACACAAGGAGAAGCCTAGCCAGCCATGGAGAGCTGCAGTGGTATA GTGGCCATGGAGAGCTGCAGTGTAGTGGCTGTGGAGAGCTGCGGTGGTGTAGTTGTGCTTCTCTGAGAAAGTGACGT CTGAGCATGGTTGTGAGTGGGATGACAGTGGCCACTGTGCAGGCTGGGAAGGAGACAGCAGTAGCACGGAGGCTGTG AAGCGAGAGCGAGGCTGGAGTGTGAGGGGCAGTGGCGGGGGGAGCGGGGGCGTGGGTGCGATGGGGTCATGGGGGAC TGAGGCCAGAGAAGGAAGTGTGGCCCTGCCGAATTTGGGACCATTTGGGCTAGGGGGCCCCCAAAGAGACCTGCCGT GTGGAGACTAGCCATGGAGAACCGTGGTGGCGGAGTGGCCGTGGAGAGCTGCGGTGGTGGAGTAGCTCACAGTGTTG GGCCCTGATCTGTCAAGGCTTTTGGCCTTTTCCTCATGATCACTAGGTGGTTGCCACAGCTCCAGCCATCACATCTG CCATGTCAAGGAAGGAAGGGTAGGGGATGGTGCCATCTGACTTTAGAAAAAGTATATGAAAACAAGAACTCTCTGCA GGCTTCTCCTCGCTCTTTGGCTACAGTTGTGACACATTTGCTGCCTGTATTGGAGCTTACATTCTAGTGAAGGTGAC ACAACAAGTAAGCAAATCACGTCACTGTGATTCCAGGTCATGTAAGGAAAATAAGGCAGAGAAAGAAGGGGTGGAGG TGGTGACTGTTTTCGGCGGCTTCTCTGAGAAAGTGACGTCTGAGCATGGTTGTGAGTGAGATGACAGTGGCCACTGT GCAGGCTGGGAAGGAGACAGAGCGGTAGAGCGGAGGCTGTGAAGCGAGAGCGAGGCTGGAGTGTGAGGGGCAGTGGC AGGGGGAGCGGGGGCGTGGGTGCGATGGGGTCATGGGGGACTGAGGCCAGAGAAGGAAGTGTGGGCCTGCCGAATTT GGGACCATTTGGGCTAGGGGGTCCCCAATTTTTTTGGCACCAGGGACCAGTTTTGTGGAAGACTAACTTTTCCACAG ATGGGAGTGGGGGGATGGTTTGGGGATGAAACTGTTCCACCTCAGATCACCAGGCACTAGATTCTCATAAGAAGCGT CCAGCCTAGATCCCTCCCATGCACAGTTCACAATAGGCTTCACACTCCTATGAGAATTTAATGCTGCCGCTGACCTG ACAGTAACGCTGGCTCACCCGCTGCTTACCTCCTCCTCTGTGGCCCGTTTCCTAACAGGCCACGGACTGGACCCCTG GTTTAAGCCATATCCACAGGAAGTGTTTTCTCATCGTGATGGAAATGAAGGGAAGATGTGATCTGATTTACTTGTGT GTGTTTTCATTTTATTCTAATCTTTATTTTTTTAACTCAGAATTATTTTACACCTCAGTTTTTATTCAGAAAAGCAT TAACCATCATTTGTTTTGCATATAAGACATGGTGAGATCACAATAAGTGGAGAATGTTTGCCATCGTTTTTCTACTT GACATCAACAGTGTAACATTAGCTTTTTTTTGTATGTTATATTTACCCATGTATTCATAATTTGGGGTACTCTTTAT TTCTTTGTATAGCTCTCATTTTTCTTTTTCCTTTTTTTTCTTTCTTTTTGTGGGGACAGGGTCTTGCTCTGTTACCC AGGCTGGAGTGCAGTGGCACTACCGTGGCTCACTGCAGCCTCGACCTCCTGGGCTCAAGCAATCCTCCTGCCTCAGC CTCCCGAGTAGCTGGAACTACAGGCACATGCCACCAGCCTGGCTAATGTTTTTATGTTTTTTAGTAGAGCTGGGGTT TCGCCACATTGCCCAGACTGGTCTTGAACTCGTGGGCTGAAGTGATCCTCCTGCCTCGGCCTGCCAAAGTTCTGGGA TTACGGGTGTGAGCCACCATGACTGGCCCAGATTGGATATTAATACAGTTTTTTTCTCCTTGAAAAGTTGACTCGTA CTAACTGTAGAAAGCTGAGAGAATGCAGATAAAAAAGGAATAGTTTAAATCATCCTACCTTATTTGATTTTTTAAAA ATAAACTGATTTTGGAATAATCTTAGATTTTTGGAAAAGTTGCAAGCAACGTATGGTGTGGTTTACTTTTTGTGGGT AATTTTTCTCCCCCCAGGAGATCACATTTGTCTCCTTCCAGTTTCTGGCACCTTAGCCCAGTAGCTCCTGTGTAGGG GTGCTAAGGCATTGGTTAAACGTCTCTTGATCGTTCCTGGTTTGTTTCCCGTTATTTTGTCTCCCTTTTGCTTCTCC CCTGGTTGGGTTTCTCCTCACTTCTCAGTGGGTTATTTCCACAATCGACCTTGGTTCTGGACCAGTCTTGACAGAGC TGCCAGAGGGAGCCTCGAAATGCAAACCAGCTGCATCTCCTCTTGTTGAAAACTCTGTGGCTTTTCAGGGAAAAGGC AGCCAGCAGGACCGGCCTGGCCATGGCGGCCTCAGGGCCCGAGGGTGGCTGTGTCCCCCACGCCTCTCCAGGCACAC CAGGCCTCATCCCCCAGCTCTCGTGGCGGGTGCCTTCACCCTGGCCTGCGTGGCTCCTCGCCCGGGGGCCCCTCCCC ATAGTTCAGTTCTCTGTGTAGGTGTCAGTTTCTCCATAAGCTCTCAGCTGCCCACACTCTCCCCCCGAAGTGTGGCC CCAGCCCTGCGGAGTGGTTCCCTAACCGCCACTCTGCCCACCTCGTGCTAGCCTGGGTGTGGGCTTGCCTGCTGGCT CTGGGCTCAGGCTGGGGTGCTGTCAGGGTTGCAGGGGTCACAGTGAATCTTCATCTTCCGTGGGCCAGTCCCCTTCC CAGCGTCCCTCCTGGGGCTGTGGGTCTGGCCTCCCCCGTTGAATGGCCCTCTCTTGAGGTTGTTGGGCATTGGTGAA GATCCTGATGGGGTCTCATCACTGGAGTCAGACGTCTTTCTTCCTGGGTCTGGGGCATGGCGGTATTACTGTGCGGG CTGTTGGGAGAGGGTCTGTTACCCTCATGGCTGCTTGTGACCTGTCCAGGCCACCACTGTGCCAGCCTTTGTGTGGC TGGTATTGTGGCTTCTAAGTTGAGCCTCCGCCTGGCTGGGTTGCGATGGTTTCACTCCTGTCAGGTTTTCATGCTAT TGAAACTCACTTTTCTCACTGCCTGATTTCTGCCTTTTGTGGGTCCCTCCTGGTGTCCGTGATGCCCTCATTTAGTC TGCAGGCATCCCCCCACCTCTCTGCTCGCCCGGTGTGCACACCTGCTAGGTCAGCCCTGGGCATCGCATCAGAGACG CTGCCGTGCTCGGTGGCCTGGGGCTTCTGTCCTGTAGAGGCCTCAGCCATTGCATTTTAGGAATCGGCACTAAACTA ACTTCTACTGCTGCCCACAGAAGAACTGTGCCAAATGCTCTGGTTGTCTACCGGAAAAAAAGGTAGTATGCAGGTTT CAGTGGCATATCAAATTCCATTTCTGTGGGCCTTTGTGGGAGGCTTGCTCTTAGCGGGGGCTGGTGGGGGTGGGGGG GGGTGGGGGTTTGTGGGGGGGGTTCCAGCTCATTGTTGTGTTCAGGTGAAATGAGGCAGGGCTTTATTTTGATCCT ATTTAGGTTGTAAGCAGGTCTGAGGAATTCACTTCCTCATGCTTCACATACCTAAATAATTAGCCAGTTGGATATGG ACAGGAGCTGGAATCTGTTGCTCATTTGTAAGCCAGATTCTGAACTCATTCATTGTTGCAGTTATAAACTGGTTGCC TTTTTTCGGTTCTTGGAATGATTTGGATGGAGCTCACCTGGTCCCCCCAGCTCCCTGCTTTGTGATTGATTCAGTGT GTTCCCATGGCTTCAGGCCACCTTTGCTGGTCAGGGGAGCTGGTTCAGAGGGCTTGAGGCCAGCCTGCCCTTTGGAT GAGAGGCCCATGCCATGATGCTGGTCATGGGGATAGCACCACAGGGCTCAGTCGGGGATATGCCAGAGCTTTAGGGT CCCTTGGGCCAGCAGCTGGGGTCCTTGGGCATTTCCTGGGTGGCGGTGCTCTCTGGGGTGCAGAGTTGGTCATTCCA GGGCAGGCAGTTCACTTGGCCACACCAGGTTTGGCTGGAAGCCTTCTCCACCGCATTCCCAACCGCTGGCCTCCCAA GTTTGGCTTCTTGTGGTCAGGGCCCAGACCATGATAACCTTGAGGACTCCTCTCTCCGGGAGACTGTCGGGGCTAAG CTGCCTGTGGCCCTCCACCGCCCTGCTCCTTCCTCCGGGTGGGGGACACCCCATCCCCCATGCCCCTACTTCAGCTT GCTGGGATTCACCCCCAGTGGCCTTCCAAGGCTCCTACCTTTCTCCCTGGCCCTGTCTCTGCACACTCCAATGTCTT CCCCTTTGGCTGACGCTTGGATTGGGGGTGAACTCGAGGTGTGGTCTGTGCAGAATGGCGTGACAGCCCTTCCAGCC CGTCACGGTGTGTAGTGCCCAAAGCCCTGGCCGTGGCCATCTCAGGTTAGACCCTCAGAGTGGCACTGTCAGGTAGA GGAGTCAGGGGTCTTATTGATGAGGAAATAGAGGCTTGAGAGAGGAGTGCGCAGCTAGTGTCTGACCAGCCCCCACA TGGGGCACTCCCCAGTCCGAATGTCTGCAGTGCAGCTTCAGGCGCTGATGTGGGCAAACCCTTCCTCTCCCAGGCCG TCCCTTCCTGCAGCTGCTCGGACACTGGCACATGTTGGCAGGCGTCAGCACCAGGGCTGGGCTCCTGCCTGGGACTC GAATCGGGAGCAGGTCCCACGGAGGCAGAGCAGCCGAGAATCAACGGTGGCCATAGCCACCACATCCTGTTTCCAGG GGCAATGGTGGCAACAGTGCCAGCTGCCCTGCCTGCTGTGCCTGGTTGCCCCACAGATGTTTCCTGAAGCTTGATTT TGGCTGTGATTGGAGCGTGGCCAGTTCTGGTTCCTGCTATTTTCTGAGCCTGCTCCTCCAGCCTTCCCAGAAGTTCT TTGAGTTGTGGAGTAGCCTTTCAGTAAACGGCATTTGTGCTTAAATTAGCCCGTGTCAGTTTCTGACACCTGCTGTT AAGAGTGCTACCTGATGCAGGTGCTCGGGGCTGGCCAGGATCCCCACCTGAGTCTGGAGCCCTGAGCCCAGGCCTCC TGACTCCGACAGCCTGCAGCCCTGCCCGAACTGGGATGATAGAGCAGCAAACATGGCTCCATGGAGAGCGAAACCCA AGCTGGGCTGTTTACTGCCAGTCTGCTGGGAGCTTTGACCCACCATCGTGCAGGGCGAGTTTCCAAGAAATGGCGAG AGAGCATCCAGATTTCTCACGTTGCCTCTAGAGCCCTGTTGTCTGACCAGTATCTTCTGTGAACATACTTTGGGAAA TGATCTCATGGACGCTTGGCCTGGATGGTGGTCACACTGGTAATTCAGTTCCACAAGTATTTTATGAGCCCCTTGTG TGTGTGATGTGTGCTGTTCTTCAGGGAGCCCTGAGCAGAGCTGCCAGCATCCTGCCTGAGAAGCAGTGGCCCGGGAT GGTGGGAGATGTCTGCAGGCGCAGGGCGGAGGCTGTGGGCCAAGATTGTGGCTCTGAGAAATGGAGATGGGAGAAGG GGAGGCAGTCACTTTGGAAGAATGGGCGGGATGGTGATGATTTGAATGAGACTTGGGGGAGTTGGGGCAGCGTACTC CAGGGGATAAGATCAGAGGTGAAGGGGGTTGGGCTAAAATGTTGATCATGGTGGGAACAGTAGTGATCCCCAAATCC TACAGAGCCTTTGGGAAGCCTGGTCTTTGCATACGTTCCCTCTAAACTCGTGCCGACGTTGTGATTTGGTCTGATTT ATCCTACAGACAAAGACAGTTGAAGCACCAAGAGTGATTTTCTCACAGCAAAGGGACAGAGGGGGGAGGCCGTGAT TTGCACCCCCTGCCTTTATCTGGCCCCTGCCACATGGTAAGTGGAAGGCAGGTGGCTCCTTCCACAGGGCTCTCTTT GCCCTCTCGGGAAGCCTGGTTTTTACCCTTGAGGCAGCAGGGAGGCTGCAGGGTGAGATGTTGAAGGAGTGTGATGT GGGCAGAGCCTCTGTGATGGGGCATGGTTTCTGGCACATCCCATTTCCATATTCTGGGGTGATCGAATGGGATCCGG TTCAAATCCCTGTCAGTCTGCACAGAAAAGGGGAGGTTTTGCAGGCGGCTCTGGAGTGTGACAGTTGAGGATTGAAT CTTGAGAGTAATGCTTCTACAAAACACTTGTGTAGTTTGCGGTCCGCTGCCCGACCCTGGTCCCTTTAAGGGACATA AATGCACCTCTCTCTCCTCGTTCTTGGAAAGACAGATGAGAGAACCAAAGAGTGGCCTTAGAAGCATATGTAAGACA CAGAAAGGAAAGATCAGCTGGAGTGCTGGTGAAGGGTTTCACAGGATAGGAGACCAGGCAGCAAGGTGCCCAGCGGA CAAGCAGGGAGGGGGCCTTCAGTGAAGGCGCAGAGATTTGGGATGCATGTGGGATTCAGACCTGCTTCATTGTGGGA ACAGAGCAGGAAGGTGCCAGTGAGCTGCCCTTCCAGGGGTCAGCAGACCTTTCTGTAGAGGGTTCCAGGGGTCCGCA GGCCTTTTCTGTAGAGGGCCAGAGAGTCAGTGTTCTGCGCTTGCAGGGCCACACTGCCTCTGTCACAACTGCTCACC TCTGCCACTGTAGCACGGAAGCAGCAGGAGATTGTGTGTAAATAAATGAACACAATTGTGTCTCAATAAAACTTTAT TTACAAAGACAGGTGCAGTGAGTGAGCTTTGCCCCACAGGCTCTAATTTGTTGCCCCTGCTCCAGCCCCTTGATGTA GGGAGCCTTTGCTTTGTTGTTCTCAGTGAACTTTGAGGGCTGATGGTTGGGTTGGAGTGGAAAGAGCCCAGGTGTGA TGGGAGCCGTGAGGATGGCCTTCCTTGAGTAGCCACGAGCTGCTGTGGGTCTTCCTGAGAAGCTGTGGCAATGGGAG GACAGAGGTAAAATAGCAAGACCCAGTGCCTAAGGAGGCTGAAGAGGGAGGATCGCTAGAGCCCAGGAGTTCAAGAC TGCAGTGAGCTAGGATTGCACCGCTGCTCTCCAGCCTGGGTGGCAGAGCAAGACCTCGTCTCTAAAAAAAAAGAAAG AAATGTACAAGTGTAAAGGTAGCCTATAGATGTGTTCCCCTGTATTCCCCAGGCTCTATTCTTCAGCCTCCTGAGCA GCTGGAACTACAGGTGTGTGCTATCATACCCAGCTAATTTTAATTCTTTTGGGGCAGGGTCTTGCTACGTTGCCCAG TCTGGTTTCAACTCCTGAGTTCAAGTGATCTTCCTGTCTCAGCCTTCCAAAGTGCTGGGATTACAGGCATGAGCCAC TGCACCCAGACTTGTACATCTCTTTAAATGTAAATTAAATGTGCTTTCCACAGCATTAGTGGAACACCCCAGCATTG CTCATCCTCTCCGGGCTGGCTCCTAACACAGGTGCCGTGGCATAGGTGCTGAGTTGAATCTCTGTTTTTGTAAGTCA TTGCATACATGCTTCATTCTAAAATGTACTTTATTCCAAATATTGAACAGTTTGTCTTCGGGATTTATTGTCTGTGG TAGTTGTTTTATTAATTGTTTTAGTAAAACAAAGTTAGCTTAGTTGTGGGAATTATTTAAAAGGAGCTCTTACCAGG TCAGCTTCCTTCGGTGTTGCGTTGGTTAAAGATAAACGAGAAGAATCGGGATACAGTAATTTTAGGAAATAAAAGGA GACTGTAGTATTGGTTGGCTGCTCTAGGTAATGGTGGTAATTTTCTGTCTTCAGAGTAATCATATTGTGAAAATGAT GCATCGTATAATTCCAAAAATGAGGAAGAGCTTTGGAATATATTTTGTGTCTTAAATAACCAGTACTTCTGTTGTGT GTTTTATATACGACCAGCCTCTTACCTATACACTTTGTCTTAAAAATTATTTCTGTTTAGGTACTTGGAAAAATGGA CAAGGATTGTGAAATGAAACGCACCACACTGGACAGCCCTTTGGGGAAGCTGGAGCTGTCTGGTTGTGAGCAGGGTC TGCACGAAATAAAGCTCCTGGGCAAGGGGACGTCTGCAGCTGAGTAAGTATGAGCCCACGTGATCCTGTATACCGCA CATGCTGAAGCAACCGAGGAGTATATGTGATAACGGCATGTTTTCCATTGATGGCAGGGTAACGCATAGCCTTACCC CCACAACCGCAACCACGAGTCCGTCTCTCAAAACAGTGTGCTGCGACGGCTCCTGCATTTGCAGCTTCCCTGTGTGT TGCCCAGGAGCTCTCCACCTCCGATGAACCCAGGGCCGTTCCCTTCATACCCTTTCATTAGTAGCTCTAAGATGTTT GATTAAGGTGTACGTTTACAGAGGAGTTGGTTGGTGATGAGCATTGAACATTTGGGTTCTTCGGCATATGTCTCTGG AAATTTAGAGACAGGGAGATGCAGGATTAATCTTGATTAAAAAAAAAAGCCCAGATCAGCGTTCCCTGTTCCTCCAG AGTCTGCCAGGTGAGTTTCATACTAGGACTGCTTGATGTTCTGGAATCCCTTAACCAGGAGTCCCAGTTGCCTTTTG AGAGCCGTGGTCAGACCCTTGGCGAGGGTGTGGCTGAGGATGGCCCTGCCAGGGGGAAGGGCCCGAAAGCCCCTTTT CAGCATGACTTTAAGTTCTTGTCATTTCTAAGAGCGTACATGTTTCTCTTTTTGTGAACAGCTCCCACTATATTTCT CTAATTGCAAAAACGTAAGAAATGTAAAGCATTCCTTAGTGTTTTGCTTTGAAATGTAACATTTAATTTCCTCCGTT CATTCTATTAAAATTGTAGCTCTTGTTATTTCTGACCAAAGAAGAAGAAGAAAAAAAAAACACCCACTCCTATGCTT GGTGTTTCTCTTAACTGGCTAATAAACACAATGCCAAGAACATTAGATAAAGAAAATACATAAGAAACATAAAAATT GATTTTAATGGGCCCCTGTCCGAGACAGCCATAATGAGCACAAGCGCAGTCATAAATATCAGCACTCGCCTCCGCTG TCGAGATTTTTCCTGTAGAGCTGCACGTTACAATCAATATAGTCTTTTTGAGACCAAAAAATAAGCAAACCTTTGAA CCATGTGAATATACTCTTTTCAGTTTCTATGAGAACCATCTGGCTGTAGTGTTCAGAAAGTCACAGGCACCAAATAA TTTGTAGGGGAAAATGTCATTTTGTCTTTTAGCATGTTCAGTTGCACAATAAATCATAGAAATTATGTGTTGGTTTT TTTTTTCTTTTGGGAAAAAGGTACAAAGTGAATTTGGGGAAGAAAAAGGAAAACAAAACAAAACAAACATTATAAGA TATGTTTAGTTAAATGCCCATTGTGGATTAGGCAAATAGGATGAAGACGCTTAATTTTGAATTTCTACTTTATGAAC TTTCTGAATCAAGTTAGGATCAGGTTCAGGTGTAAAAACAGCCGTGAAATAATGCTAAATCCTGAATAGGATCTGGC TGATTGCTCTCATGAAAACACACCTGTGGACAAACCATGGGAGCTGGGGCTGGTGCCTTCATGGCCCCTGGGAACTT GGGCTCCTCTGAGCTTGGGGTCTCCAGATGGTGGTCCCTGCCCTGGTGCTTCAAGACAGCTCCTGGGCCTCTGCCAC AGGTAGGGGAGAAAGAAAGGAGGGCGTGTCATATATGGGCTAAAATTAGGTGTCTTGTGAGTAAGGAAGGAGTGGGA TTTGGGGGGCAGCCAGCAGTATTTGTGTGTAGTTAATTTTAGTGGGGGGAGCATTTAAAAGTTTGAGAATAGTATAT AAAAGTATATGAATAGTCAATTAAGTTTAACATTTTATCATTTTTAAAATTTAGGTAAAAATTACATAGAAGTCAGA TTTCAAGTGTACAATTCATTTGACAAAAGTGTACAACCGTATAACTACCGCCGAAGATATAGAACATTCAAGATATG GAACATTCCAGAAAATTCCCTTGTGCCCCCTAAAGTTAGTCCTCTTTGTGAATTTTTGTTTCCATTTCTCCAGAGTA AATTCCTGAGTGTGGAATTGCTGGGCTGGATGGCAAATGTATGTTTAACTTGACAAGAAATTGCCAAAACTTTCTTC AGAGCGGGATGGAATTTCCACTAGCAGTGTGTGAGTTTCAGTTACTCCACGTTCTTGGCAGCACTTGGTGGTGTCAG TTTTTCAGATTTTAGCCATTCTGGCTGGTATGAAGTATATCTCATTGTGGTTCTAATTTGCACTTTGCTGATGACTT ATCCTGATGAAGTCTTGGCCATTCCTATGTTTTCTTTGTGAAATATCTACTCAAGTCTTTTGCCCATTTTTTTTTTT TATTTCTTTGAGATGGAGGCTTGCTCTGTCACCTAGGCTGGAGTGCAGTGGTGCAATCTCGGCTCACTGCAACCTCC GCCTCCTGGGTTCAAGCAATTCTCCTGCCTCAGCCTCTGGAGTAGCTGGGATTACAGGCACGCACCGCCACACCTGG CTAATTTTTGTATTTTTAGTAGAGACAGGGTTTCACCATGTTGGTCAGGCTGGTCTCTAACTCCTGACCTTGTGATC TGCCTGCCTCGGCCTCCCAAAGTACTGGGATTACAGGCGTGAGCCACTGCGCCTGGCCACCTTTTGCTCATTTTAAA ATTAGAGGTTTCTTGTTTGTGTTTTTGTTGATGTTATTGATTTGTGGTTCTGGATGTAAGACTTTTGTCAGATGTCT GTATTGTGATGATTTTCTCACAAGTTGGAGGCCTGCTTTTTTGTTTTTCCAATAGTGTCTTTTGATGAGTAGAGGTT TTAAATTTTGAACAAGTCCATATGTCTTTTTTTCCCTTTATGTTTAACACTTTTTGTGTTCCCTGTAAGAAATATTT GTCTACTGTAATGTCTGAAATAAATTCTCCCGTAATTTCTTTTAAAAGCTTTGTAGTTTTTTTTATGTTTAGATATA TGTGATCCATTTTGAATTACCTTTTTGTGAGGTACAGAGTTTATTTTTTCCCTGTAGCTATTTAGTTGTTTTAGCAT CATTTGTTGGAAAATAAAAATTGTCTCCTTTCTTCATTGAATTACCATCAGTTGACCAAGGGTGTGTGAGCCTGTTC CTGTTTTGCTCTGTTTGGTTCAGCCTACCTTTGCCTGTTCTATGCCCGTTCCATGCTGGCTTGGTTAGGTAGCTTTG TGATACATCTTTTGTTTTGTTTTGTTTTGTTTTGAGACGGAGTCTTGCTCTGTCGCCCAGGCTGGAGTGCAGTGGTG TGATCTCGACTCACTGCAAGCTGTGCCTCCTGGGTTCACGCCATTCTCCTGCCTCAGCCTCCCGAGTAGCTGAGACT ACAGGCGCCTGCTGCCACGCCCGGCTAAATTTTTGTATATTTAGTAGAGACAGGGTTTCACTGTGTTAGCCAGGATG GTCTCCATCTCCTGACCTCGTGATCCACCCACCTCGGCCTCCCAAAGTGCTGGGATTACAGGCGTGAGCCACCGCGC CCGGCCTGTGATACATCTTGATGCTAGGTAATGTGAGTCCATGAATCCTCTAAATTTATTTATTTTTGAAAGGTTTG GCTTCTATGTCTCTTAAGTTTGCCTATTACTTTTAGAATTCAGGTGTAAATTTCTGCAATGATTCTTTTTTGGATTT CGACTGGGAGTTCATTGAACCTGGTTGTCAATTTTGGGAGGGTTGACATCTTGATACTGAAGTGTTCAGTTCTTGGC TTTGACTGGCGTCTGCATTTGTTTGGGTTCTTTCATTGTCCTCGGCAGCGTTGAGTGGTTTTTCAGCCTGGAGGCTT TGCTTATCTGGCAGACTCCATTTCTAAATACTTTGTTCTTGATGGTATTCTAAATTAGGTTGAAACTTTTTAACCTT TTAATTTATTGCTGCTTGTGTTTAACAACTGACTTGTATGTTGATCTTATATTCTGGGATCTTGACTTAATTCATGT ATTTTTTCCAGGGTTGTTTTGAGATTGCTTAGGATTCTGTATAAAAATTCATGATGTCTATGAATAAAGACAATAGG CTTCTTCCTTTCTGATTGTTATGCTTTAATTTCTTCTATTGAGCTGTCCAGAACCCTCCAGGATGACTTCAGTAAGT GTGGTGACAACAGACGTCCTTCCCTGTTTCCTGGTTTGGGGGAGCTTGCAGGATTTCACCAGTGAGGACTTGCTCCC CATGTTGGGTTTCAGAGGCGTCTTCCTCACTGTGTGGAATTTTTGTCAAATGATATTTCATGTATGTGTTGACATAG TCATGTGCTCTTGCTCCTTTATTCTTTAAATTTGGTAGAATAAATTTTTAGAATTTTAAACCAGGTGTATACTTTTC TTTTTTTGGTACTGTCTTTGTCTTGTTTTGGAATTAGTCTCATTCTGATGACAGATCAATTTGGAAGCATTCCCTCT TCTATTTTCTGAAAGAGTTAAGTGTTGGATTGATATTACTTTATCTCCAAATTTTGGGATGAATGCACCTTGGATGC CATCTGGGTCTGGTGTCAGAAGCCTTTTGTGAAGCTTCCTGGTTTAGTAGAATTGGGTGCTGCCTTCCCTCATCTCG GGGTGCTCCCACCTGGAGCCATCTGTGCTCTGTGTGGCTGCTGTCCTCTGGTTGCTGAGTGACAGTGGCTGCTGGGA TTTGGGGCAGCATCCTGCTGGCTCTGGCCCCTGCCCTCACCCTTCTCCGAGTGCCTATGAGTCCCAGGGTGGTCCTT GCTTCCTCTGAATGGGTGTTTCTTTAGTAGATAAATTACAGGTTGACCACTGGGCCTTCTTCAGTGACATTGCATCT GTGCTTGTGTACAAAGTGTGATTCCCGCTCCCCAGGTGAATCCACTTACCTGTGTGTGGCTTCGCACGGGGACCAAT GACTTCCTTGCATAGTTACCTTTCCCTCCCTAAAAACGTGTGAGAAGCACATCAGTTGGGAAAGTGGTATATTTTCC AAAAGGGAAGCAACATAAGCTGCTTTTAAGTTAGTTTCATAACTGCCTGACAGTATCAGGTTTCTGTGAATGCCATC ACGTCCAGGAGCACTTGGCCTCTGCAAGAGCGGAGCAGCTCCCCCGCGCCATCTTCCCGCAGCTGCAGAGCTCTGAC CTCGTCAGATTCCCTGCAGTGCCCACAGTCTAATGAGAGGGGCCAGGCTGTATGTGTTTACTTTTCTAATGTTATTT TGGGGCTTGTTTAGTTTTCCATAATGTTGATTGCCGTATTTGATTACTTGAAAAAATGGACCGTATCACCCTGGGTC ATACTTAGATTACAAAAATAGTAGCAGTAGGACTTGTAGATCTTGGAAGCTGGGGAGCTAAGGGGGCTCATCTTTCA CTTACCAAGGGCTTACCCTTGTTTTTCATTCTTTTATTTTTTTCACATAAGCCTTTTCAGAAAAATATAGGAAAAGC TGATAGAGTCCAATTGAACTAAACCACTTTAAAACTTTTTGTATTTTGGAAAATGTGGGGACTACACACAGACTGTC TTCTACTGCAGAGGCTCTTTTTCTGTCTTCATGGTAGGGATCCATTTACAGGGTTTGTGGGTCTGCTTTTGGCATCT GGCCCAGCCAGCCCTTGGATTGAGGCTGGGTGGGGCATGCAGGTTGGCCCTGACCCTCTTCTGAGCACCTGGCTGGC TGGACAGATAGGGCCTTCAGTCACCAGCTAGGCTGGCCTGACCCTAGACCCTCTGGAACGTGAGAACGTATCCTGCC GGGCCTCTGTGTTGAACCTGGGAGGAGAGTTGTCTGATGTCATTGGATGCCCTATGAGTTTCTGCCCTCGAAACGTG CCCCATGGAAAGCGTGAAGATGCCTTTGAGCTTCCCGAAGGGCAGCTGCAGCAGGAGGTGACCTGCGTTTTGCCTCT CTCCTGCTCATCTCTAAATGTGTTCAGGAACTCAGTCGACCCTCAGCCGTGCATGGGGTCCCGGCTGGCTTGGTATG TGCACCTGCCGAGTCTGTGCTGCCAGTTATCCCCACGGCCTCTCCGTGCAGGTCGGTCTGACCAGTGCCAGTCGGAT GGTTCCTCAAGGTCCACGGGCCATGAGAACCTTCCTGGGAACTTCACGAATTCGTTTTTTTCTGACAGATTGGGAAA TTAATACAGACACTTGCCGCATTCACGTTACTATAATTGATCTTTCAAGGAGGAGAAGAAACTGTTCTTAAGGACAA ACACTTAGAGAAGCACTGCCAGTAAAAATAACATCCAAAAAAACGCGCTCTGTAGGACTTTTACCTGCGGCAAGTTC GGCTGTCTCATTGCTTTTCCTCAGTGCCGTGAAATAATGATTTGTGTTGTATGTGCTTAAAAAATGTGATTATTTTA CTTTATAGAGTGTTCCTTAAGGAGGTCTCTTGAGTCAAATAAGCATGTTTCTGTGAGGAAAACTTGCCAGGCGCTCC TTGGCTTTATAGAAATCGTCTGTTTTTCAAAAACGAATGGCTGAATGCCCTGGCGAGCCTGGAACCCCTTGTTCTTG CCCCTGCTGGCTCTGCGATTCCATGTCGGTGAGGCCTGTCCACGCTGACGGATGTTGCCTCCCGTTTCTGCCTGAGA GGGGTAAATGATCAGTGGTGGATAGGGAGTTCCTCTTGTGACTTATGTTATCAATAACACAAATTGTGCATATACTT CACCCCATGCTAAACATTTATAGTGCAGTGTCCTTTAGAAGCCAGACAATGGCCTCGTCTTTCAGGTTGATGGCTAA AAAGACAAGACAGGAAGCCAAGGTGTTTCTTTTCAGCTCCACAGATCCTTGTGATGAATTATGTTCCGTGACGAGAG GGTACGATGGGGGCCGACTGCTGGCCCCCGGACACACAGTGCTGCACATGGGGCCTGCAGAGGTGTTAGCGTAGCCC GTGATGGTGGCAGCTCTTGGACCTTAGTAAAATAAAGGGAAGCAGCATAACATATGCTTTTAAGTGAGGGTATGGCG TGAAAACCCTGCCGTTGTGTGATAGGAGACGGGCTCTGCAGGCTGAGCCTTCCAAGTCGTGAATGATGAGGTTTGCT GTGCTCCTCTGATTGGCAGCTGAAAGGGCAGGTCAGAGCTCCTCTCTACAGCACTGCGGAGAATCCCAGGCTGTGGA GCACTGGCCCATCCACCCCAGGACAGAGCTGCCTCGCCACAGAAGAGCGAGGTTCTGGGATTCACTGTGAAATTGTT GTATGGTTTCTGATCTAGAAAATCAAACCTAAACAATGCGCAACCTCACCAGCTTCGGTGCTCTTGATGTTTTCTCC CTGCAGCTGGTTCTTTGCTGACCTGGGGGCAGAGTAGACAGTGGTGGCAGCCAGGTGGATAGTATTTAAACAAGTGT CACTGAGATTTAGGGTCCTTATGTGCTGGACTCATCTAAGAAAAAAAAAAGCAATGCAAAAGTTTTATTTCTGTCAA TTTTGTAACTGTGTTACAAGGAGACAGAGATGGCAAACGTGTGTGCCTGTGTCTGTGAGTCAGAAGCAAACTTGTTC ATTTCATTTAATTTTAAGTCTGCCAGGAACTTCTTGGAAGGAAGAAAAGAATGGTTGAATGACAGCTACAAATAGGT GAGTTCAGAATATGATCCAAAGTGTCAGAGCAAAAGATTTTTTAAGTTTCCAACATTTGGAGTAAAAGAGCACTTTA GAGTAACAGGTAAATGGGTTAACTTCAGGCAGAGAGAGGTGGATGTTTCTGCCTCTCTGGTATGTTAAAATAAAACC CCTTAAATCCCCTGCTCTTAACAATAAGTTTCCATTTAAGAGTCATTGGGCTTCACCTCTGTTCGGCTCTGTTTGTG AAGGGAGGGCAGCTTTTCTGGGGAGATCATGCCCCTCTCTCCTTTATGTGGCCACAGAGATCCTTTCCCTGTTTTCA TGAGGAGGAAACATGGAACCTTGGCACAATCCAGTTCTCATCTAGAATTACATTGTATGCTGTGCAGGCCAGGGGAG GAAAGATCCCGGAAGTGGCAGCCACCGGATAGGCACGTTGCCGCCACAGCCAGCATCGGCGTCTGTTCAGGTTTCAG GAAGGTCACTTCTTGGCTCAAATCCCTGGTATTTCCAGGTTGTCTTCAGACTGCGGCTTTCACCCTCAGGAAGCCCC AGCATGGCCTTGGGCCCAGCTTAGAGGCTTGTGGGGGTTTGGGTGTAGCCCCGTGCACAGTATCCTGTCTAGGATTC ATAATCTTGCAACAGCCATCCCTGATGGGATTGGGTAAGATGACAGTGTCCTTTCTCGATGGCCCTGGTGCCCTGTG GAGTTGGGTCCTGGCCCTCACTACCCTGTTGCGTGGGTGGGCCACGCCCCTCCTGCTGATCAGCCTCTGTTGCAGAG AGTCTGGCCCTGTGGACCCCTGCCCCCGGCCCCAGGCCCTGTGTCTGCGCTGGGCTTGCATTGCCTGCCTCTCGGTC CCCTCGCCTCATTGCAGTTTGGTGCTTCTAATTAGTTGTGGTCTTGAAAAGCTGGTGAACAGGCTTGTCCACATTGT ATGGGAAATAAGGTTCAATGAACCCCTGTGAGAAACGCCAAGTGTGGGATTTCTGACAGTATCCCACAGAAACATCT CAGAGCCCATTTCTGTTTCCTCCTGGAGTCTCTGTTTCTCCCAGAAGAGGCGGCGTGGCCAGGGGTCAGGCAGATGT CAAGGAAGTGAGGACTCGGAGGGACTGGCACCTGTGGAATAGCCCCTGCTTCGTCTGTCTGGCCTGGTGGGAGGAGG TGTCCTTCCGTCACTGCACTTCGTCCTTGGGACCCCTCTGTGAAGTTGTTGGCATCAATGTGCGTTTACCAATAAGA GAAATGGAGATGGGGTGAGGGATGGTGGGTTACAAGTCCTGGGGAAGACTTCAGGGAAGAAGTGGGTATTCCCTCTA CTTAGCTGAACTTGCAGTAGAGACTGCCACTCTGGGAGTGACTTCTGTTCTCCTGTGAAATCCTCTGTGACTCTTGC CTGCAGTTGAGGGAGCCTCTGAGACCATCCTGATTGGGATTTGGTCAGTGGGAACTCAAGTCAGTATTGTCCTGGGG GCCTGAGTGCCAGTGTCTTAAAAATAGAGTGTCCTCCAGGTAATGTTGGATTTGGAAGTTCAGGCTCAGCACTGGAT TTGTCCTCGTGATATCACTCGCCTACTGTACACATGAGTCATTGTAAAGTTACGACATGAGCTCGCATACGTGCTGG TATTAGAGCTCCTTGGACGGTGATGTGCTGTTTTAGAGGAGAAAAAGCTGAGATGAAACAAAAATGGTGGTGGCAGT TACCCAGGGCCACGTCTGTGGATGCCATCCTCAGGATGCCATTGCATCTCGGGCCAGCCTCCCTCTGCTTTTGCAAA AGCACAACCGTCTGGCCTCTACGACCACCAACACACACCTCTCATACTCTGGAATTTGATTTCTGGAATGCTTTTTT ATTCTTCCTATATCTGTAAGATTTTCTGATATTTAAACTGGCTGCAACTAATGAATAGTTTTCAGTTTTGTTTTAAA AGTGTTCATGTCATTTAAGCTCTAAACTCTCACAATATACTTGGTTTGTTAAATGTAAAATTTTATCAGTAATTAGA TCAGCTCCTTGATATTAATGTACAGGGTAGCTCTTGCGTGCTGATCAGTTCAGAATTTAAATTGAGTTAAAGGTTAA ATGAGTTTAATTTTTGAATTGAACTGAAGCATTGTCTTGTGGCAAATTCATCCAGAATTTTTATTTCCAGTTGCTGT ACAAGTGACGGAAGTGGGTCTTTTTGCGATAAAAACATTGTGAATACATTTAGGATTCAGTGAGCTTCTGTCATTTA CTCGCTCATTCTCTAAACGTGTGAGTGCCCTGCTCCGTGTCAGACCAGATGGCAGGCCCTGCATGGGGTGGGGAGGG GCCAGAATGTGCAGCCCCACCTGCAGCCCAAGCCCAGCTCAGGGGCAGACAGGGGCAGACGGGGAAGTGGGCTGTTC CACACCTTGAGATTGGTGAATTCCTCAAGACAGCGGCTGGCAGGGGCCCAAGCCTAGGTGACTGGCCACAGAGCTGA ATGGGGCTGACCGAGTTAGGACATCACTGGGTGGTGAACCAGGAGGCCCTGGCTGCTGACAGGATGTAGGGGCATGG AGGGGTAGCAGAAGGAGGCAGTTAAGAGGCTGCGAGCCCCCTCTTGGGCATCTGACGGGGTGGTGCTTCCTCCCAGG GCTAGGGGAGAGGTGAGCAAAGAATAACCTCAGGAAAAGAAATAAAAGACCTGGAGGAACAGTTTAACAAGTGGGTG GCCATGAAGAGCTACCAATGGAGACTGAAGGAAAACCAAGAAGGACCAGAGAGAGAATGGAGCCAGGAGGCTGGAAG GAGAGAGGCGGGTTTTGTGTCAGGATTGCCCAGAGCTCCCCGGAGAGGAGCATCTCTCCTCTCCATGGCAGAGCACA GAGGGTGGAACACTGACGTCAGATCTTCTTCAGGCCCTGAACGTAAGAATTCCTTGGTACCTGGTCTGCTTTGAGAC TGACATGCTGTGTTTGATTAGTTTGACTTCGGGATGCCAGCTAAATGCTGTTTGCTCAGCATATGCCTGAACTGGAA ATGGTTCCATCACAAAACAAACTGAATCAAATAGAACGTGGGGGTGGCTCAGTGGGGACACAGAGCCAGAGGTGAGT CTCCTTCACCCCAAACACCCCAAGTTCTAGATTTGTTCTTGGAGCTCTGTGTCAGGGTTGGGGTGGACAGGAGGGAG AGAATATCCTCCGCAGAAACACAGTTTATCACACTGCAGAATTCTTAGTGTTCTTCTGACAAGGACGTTTGTTCAGT TTCCCTCTAGACTGGGCCATGCTCTCTAAATAGAACTGAAATCATAGTTACAACTCAAAGGCAGTTGAGAGAAGAGT TCTAGCTTTGTTCCATAACGTCCTGTGTCTTGATTTCACTGTATCTGTCTGTTCATCCTGCTGGGGGCGTTAACCCA CCTCTTCTCGTACAGGGTCATCTCTAGTTTCACTGAGGACCTCTGAGAACCCTAGTCCCTCTGTCCATACACATCGG CAGGAGACTCACCTCCCCCCTTTCCTCCTGTACCCCATCTGATCCACATGCACAAAACACACACATGCAAATATTTA AAAGTAGAAAGGCTTATGGACTGTATGACTTCAGTACACCTGCAGATTCATGGAGTGGTTCTGGAGAACATTGGTCC TTCTGCCGGCCTGGCAGCTACTTTAATTTCTGGTGTCCATTGGCAGAATACAAGCTCATAGTGTGTGCTGGGGGCTG GGTGGGGCAGGGGATTACCGTTTTCTTGCAGTGATTGTTCTTGATGGAATCCACATTACTGCTTTCAAAGCTGCTTT TCCTGTGCCTTCTTCCAGAAAGCTATCCTCCCGTCCTTGGTTGGCTGGGGCAGGTGAAGCATTAGGAAATGCTCTGT CTCTGCATTCTCTTCGAGTAACACTGGGGAGTGTTCATGGCTTCTGGCAGAACAGCAATGTTAGCCTTCAGCTGCTG AAGCAAGCCTTCTGCTCATTCTAGAGGCATCTTCTAAACAGATCTGCACTGGTCAGGTGGGGCTGGAGAAGGACTGG GCTGAGAGCACAGCTATGGGAGGCAATACTTGTTCTTGTTCCCAAGAAGCTGATGTGTCGTCCGGTGAGGTCTCTCG TCAGCCGAAGGTTGGCACAAGGTCGCCAGGCAAGGGCTTTTTGTATGTGAAGTATGCACATTCTAGCCACAGAGTTT TCTGCCAGGGTGTTTAGACAAGCTAAAAGTGGAATATAAACTGTATTCAATTTGCTAGGTTGCAATTATAAGGACAA ACATGTTTTTAAGACTTAACCCATTATAGTAGGAAAATACAAGGCTTGGGGTATGCTGACTCTTTCCCTTGTTCGTC TCTCAAATAGAAAAATGGTGTGTGTAAGTAGGTGTTTGTATATGTACATTATAAGACATTCTAGAAATTTCTGTCCT ATTTCTTTAATATTCTTGCCACTAAAAATATATGCTCCTGAAACTAGGGAAAAAGAAACTGTTTATCATTCTTCTGA ATTATTACAGATACCCCAAAAGTAGCCTCCTCCAAATAGTGGTTTTTCTTTTCTAAAAATCACCTGGAAAAATACTG AGCTTTCTGATCTTTATTTTATCCTTTTCAGTGGCTCTGTTTGGAAGAGTTATTGTACAACCCAGCACTGCTTTTAT TAAAAAGAAAACTGGGCCACTCATTTCCTTCAAAGTTTTTGGTGTGTTTAAATCTTCTCTAGGTAAACAAAACTTTT ATTGAAAATAATATTTTGAGACCGTGACACCCTTCTCACAAGAAGCGGTGCTGAAAGCCCCCTTGTTGGCTGGTCCT GCAGGCAGCCTGGCGTCTGCTTTGCCTGTGGGCCGTGCAGGGTGCTGGGGGCAGTGGGGAGAGGCGTCAGTCCCACC TGCAGCTTTCGGGGGGGTGCTGGGGAGGCAGATTTGACATGAACGCAAATAAGTGCAAGGACCATCAGGGCAAAGAG ACCCAGGAGACCAGGCCAGGGGAGGAGGCAGAGAGCCTGGTCAGGCCAGGCTGGTCCTCCAGGTCCAGGCCCTGCAG GATCCTGGAAGGCGTGAAGATGGGCTGGAGGTGTGAGCACCAAGAATGGGGCGCGGCCTGACCTTGGGTCTCTAGAA GTTACCCTGTGGTGGCATTTTCTATAATTGATGTCATAAATAGCTGGCTTCTTTGTTAGAATTCATTTACTTACTAA GTTTGGCAAATATGTTGAAGGAAAGAAATGTGAGTGTGTGGATGGATTTCAGCACGTTGACATCAGTGCCTGTCTCC TTGGTGCCTGTTGGAGACACCGTGTTGGAATAAGGATGAGGTTCCTCCCTCAGAATGGTTGATCCATTTCCTTCAGT TCTTCTTCAGTTTTGCAGCTTTTCTCCCCTAACTGTAGCAGACCATCTCCTGGAATCTCATATTAAAGGCATAATTA ATCATGTGATATCCTCTCGTTTTTTGCATCAGAGTTTTCAAGAGCCCTCCAGATTTTTATTGCCTGCAGATCCGTGT GCTTAGACATAGTACCGTCAGCAGTAAACTGGCTTCTGGAGAAGAAATTGACAAACAACTTTATAGTTGCATTTTTA GCCTTAATATCTGAGGGCGAGAAGAATGTTCAGGATTTATGAAGTTCCTGAATTTAAAATATAAGCAATAAAATGTT CCTAATGTGGGGTCCCCATCTGCATCTATTATTACAAACTCTTAAAGTCACTTTAGCCTAACATGTCAATAGGAGTT TTCTTCTCTTTCTCTTTTCTTTTTGACAAATTGTGTCCAAGCAAATTAGTTTGCTTGCATGAAGCGTCATCTGTGTT TGCAAGCTGGAACAGTTTTGCTGGTTAAGCAGTCTAGAGATGATAAAATTTTTTGTCCGAGTATGACTTTGGTGGTG AATTTAAAAGAATCACGGCCGCTGCTGGAAGCTCAGAGGAGAGGTCCATTTTAAAGGGCGTCTGCTGCACTCAGCCC AAAGCGTTACGGGGATTGTTTGAATTGACAAAATGAAAATATGTATTATATTTTCTCTTTTTAAAATCCATTTACAG TTTATTTGTCTGATATGGAGGAGCAGAGGTATAAATTACAGAGAAATTGTCATCATATCCTCTGGATGATAAGTAGG GCCAGGGCTTCTGTTTATTTCGGTGGTTGGTTACCCACTCAGGACAGTAAAATCACAAAGTTTTAGCTAAGGAAGGA GAAATCCCTTCTGCCTGTATAATAAATGTTGTTTTGACCCAACTACACTGTTTCTTGTGATAAAAATATGTCACTTA AGGTTTCCTGCATCTGCCATTTGTGAACGTACAGTTGAGCGACATTAGTTGGTGCCATCTGTGAGTGCACAGTGCAG CAACTTTAGGGGGTTCACAATGTTCCGCAGCCATCACCGCCGCCCAGCACTGGCATGTTTCACAGTGTTCCGCAGCC ATCACCGCCACCCAGCACCGGCATGTTTCACAGTGTTCCGCAGCCACCACCGCCGCCCAGCACCGGCATGTTTCACA GTGTTCCGCAGCCACCACCGCCACCCAGCACCGGAATGTTTCACAGTGTTCCGCAGCCATCACCACCACCCAGCACT GGAATGTTTCTGTGTTTTGATTCTTTTTTTTTTTTTTTTGAGACTTAATCTCGCTCTGTCACCTAGGCTGGAGTACA GTGGTGCGATCTCGGCTCACTGCAACCTCCATCTCCCAGGTTCAAGTGATTCTCGCGCCTCAGCCCCCCAAGTAGCT GGGATTACAGGTGCACGCCACCACGCCCAGCTAATTTTTGTATTTTTAGTAGAGATGGGGTTTCACCGTGTTGCCAG GATGGTCTCGATCTCCTGACCTCAGGTGATCCACCTTCCTTAGTCTCCCAAAGTCCTGGGATTACAGGCGTGAGCCA CCGTGCCTGGCTTCATCTCCAGAACTTTTCTGTCTTGCAGAACTGAAACCCGTACCCATTAAACACTAACTCCCCAT TTCTGCCAGCCCATCCCCTGGCACCCACCCTACTCCTTTCCGTCTTGATGCTAACACACCTTGTGTGGGTTCCTCCA AGTCAAGAGAAGGAGTGTCCAAAGACCCCTGAGACCAGAACCCTCTGCCAACGTTGGTAGTGCTGTTTCGGCCTGGA GGGCAGGTGGGCCTCTGGGCACTTCTTCGTCAGGACATGGAGCCATTTCGGGTAGGGCCCTGGGTCTACCCTGTCCC AGCCCCGTTCCCTATCAGAAACCCGAGGCCCAGAGGTGCAGGGCAGCTGCAGCACCAGGGTGGACAGATGTTGATGA TCAGTGGGAAGGAATTCAAAGAAATAGAACATTCTACACAAATGTGGGGAGGTATCTTTAGTAAAAACAGGACCCAA GTACATGCAAAGTGCACTGTGAGGGAGAGAGATGCTCTGAAGAAAGGCACTGTTTTTACATTAATGAGCATTTAAAT GATGCTGGGTTAACCATTTGGCTTTTTGTACCTTGTCACCATCTCCTTTATGGTGTGATTGGACTTTCGAGAGAAGG TGGAGGAAGGTTAATAGATAGGTAGGAGAGTTGCAGGCAGGAAAGACAGAAGGCCCCGGGGATGATAGGTGGGGCAG AGGGAGGGATGGAGAGGTGACTTCAGGAAGAGCATCAGCTGCCGCAGGCCAGGGCACCCTCAGGAAGCAGGCCCCCC ATGCTTGCAGCTGCCCCCGGGAGCCCTCATCCCCAGTCTGACAACTCGAGGTAGATCTGGGTTGCTCTTTATTTTTT GATGATTTTTGCATAGTTTCAAGGATCAGTGTCTTCTACACTGATATTCTATCTCTGTAAGTTTCTCCGAGGTATTT TCACCTGTACTGACATTGTAGTCTTTCCTTACTCTCTTATTTATTTATTTACCAGACCCCACCACTGGGTGCAGTGA TCAAAACCAAAATATCAAACCAGCACCAGAAACTCTTCAGCAGACCGGTGGAACATGCCTTAGAGAAAGCCAGAGGG GTCAGGCATAGCCTCCTGTGCCTGGCCTGCGCTCGCCTGGGAAGGGCACCCCTCGCTGTCCAAGAGTGACCCCAGGC CACAGAAACCACGCTGAGTACCAAAGGCGAGTCCCCAGGGTGTGGGCAGGCTCCTTGCTGGCATGTTCAGCCCAGGC TTGCCGGAAGTGGGGCTCAGGCTCTGTCTGTGGGATAATGATCAGAAGCTTGGCAGTCATGGGACCAGGATAAGAAG AACCAAAGGACAGCTCTCCTCTCATGGTTATGGGCCAAAAAATAGGTGATTGTTTTAACTGTGGAAACTGACCCACT AAGGGGGACCTGCTGAGAGGAAGGCCAGTGGGGCCGCAGACAGGAGGGGAGGCCACAGCCGCCCACCCGCTCATGGC CGAGCGCCCCGTTTCCTGAACATGTGGGAGGGGCAGGCTCCCCACATGGGAACCACTGGGCCGTGACTGTGGCGCTC CAGCTGCTGGCAGTGCACACCCGGTGGGGCCCTCTGTGGCTTGAGCTGCGTCACCATGACGCTGCATGCCCAGGTGG TCATCCAGGAGGATGACTCTGTGCAGACCTGTGTGCCCCCGGGCCTGGGCCTCCTGCACCCCAGAGGTCAGTGACAG TGGAAATTGCCACAGACGTGGGTTTGTGGCCTTCAAAGCCTTCAATGTCACAGAATTCTGAGTCTTTAGCTAAATTA AGTACATGTTCACGTGAGTGTGTGATTTCTCCATGAGGACTGAGCCTCATAGTTTGTCCCTGTACCGGTGTCAGTTC ATCGCTTTGGTCTTGGTGGAGAATTCTGCATCGCAGGCACATCGTCTGGTTAGCGGGAGACATGTTTGGCCAGCGGT GAAAGACTGCCTTGCCCGAGTTCCTGAGACTTCGGGGCTACTGATGGCAGGGAGAGGCTGGCCGAGGGCAGGGCCAC GCGGTCCCCTTCTGCTGATGTCCGTGAGGACTGGGCACAGCTGCCAGAGTCAAGGGCACGGAAGTGCAAGCAGTCCG TCCTTTTCTGAAGCCTGCTCTTTTTGTTTTTCCCCACGTGTGTCACCTTTTAACGTTTTTTTCCTGGCTTTTTCATT AATGTATGTAAAAATATTAAAAGCCCTTGTATATTACATGAAAAACTATGTCAGCTGCCTTGCTTTTTTCTATTTAT TAAAACTTTTCAAGCAGTAAAAACATTTTACTTGTTAATGAGGGAAAGTGGGAAAATTAATGACATAGAGCTTGGAT CCACCACGATGTCTCCTCTTAACCATGCGCAGCCAGCTTCCCACCGCATGGAGTTTAATTTCTGCGATAGGGTTCAG TAAGCAAATGCAAATAGATTTAATGATTTGCATCAGTAGATCATTTCTCAGTTTAATATCCTCTGATATTTTGGGTC AAGGACTCTTTCTTTGGGAGACTAGTATAATTTCTAGAGGTAGAATTATTAATATTAGTGATATTACATTATGTTGA AGTGAAGTGAGATAGATGTATCATTTTCACAAAGTCAGATTTCTGCGTGACTTTTATTCATTGTGGTGCAAAGGAAA CCCTGGCTGCTGAAGTAGACTTAGGAGCAGGCAGAATTCCTGGACGCAGGTGCCTTGGAGTGAGGTGACCCTCTCTG GCCCCCAGCACCTTGCGAGCAGTGGTTTTTGCATCTGTTCACCAGCGCATGTGAACCTGTGGGCCCGGGGGACACGT GGTCCATTTTTGTTGAGTACGTTCAACAAAGGAATGAATTAAAATGAGAACTCTTTTCAGCAAACGCTGATGGAGCT TTCTGTGTCTCGTGTGTGGCACCATGCTGACGGAATGACGAGAAACCACAACCTGGCCTCAAGGAGCTTTGTCTACT GCGGGAGACAGAGATGGTGGCAGATCAGTCCTGGAGGCACAAGGAGAGAGTGGTAGAAATACACAAAAGGCATTGAA GACCAGTGGAGCATCAGGGTCACTTTCACAGGGAGGAGGCCAGGGGTCTTGTTGCCTGCCTGCCCCAAACGTGGCCT CCATTCCTTCTTTCTGTCTCCCGCTGGAAAGGCTGAGTGCAGGTGCCAGCTCTCTCTGCCTCACTGAGCGGGTATGT GTGAGAAGCGTAGGCCTACTGGCAGGCTCCTGGGGAAGACGTTTCTCCCCTGATACAAAGAGCAGCATGAGGAAGAC TCTCCCGGCTCCCCAGTCTGCGGACACAGTTGTGTGAATGTGATTGCAGGAGGGAGCTGGGCAGCTGTTATGTGACC ATGAGGGCGTGGGACTGGGGCTGAAAGGTGGCGTGCTGCCAATGAGAAAGATGGGGAGAGCTGGGCACCTGCTGGCC CTGTGGCCGGCCAGGCCGCCTCTGAACACCTATTCTGTGACCTGATGTGCATCCTCATCACACACTGCAGCCGTGAG CAGCACGCAGGTGATGACGGGTCATACGCCATCTGGGGACTTGATGCTCCTTTTAGCAAATACAGAAAGTACAAAGG AAATAAGAATCAGTTCCATTAATCAGTGATAGTCTTTTGGTGTAATTCCTTCTAGCTTTTTTTTTGTTTTCATCGAC ATATTTAAAATCAGTTTTGAATTCTACTTAATTTTATATCTTAAGCATTTTTCTTGTTCTGACATCTTTTTAAGCAT GATTTTTCAAGGTCTGTGCTTGGTTCTTTGTTAAGGCCTGTTGCGTGGTTGTACGTGGTGGTATTTTAACTGTCTAT GTCCTGGTGAACCTCTATGTTTTTGTTTCTGTTTACTGGGCTGCAGTAAACAGTCTCGATGAACACTATGTAGCCCT GGGTGAGTGCTTCCTGAGCAAGCTTGCTTTTTGGCCTTTCTCCCTGTAGCTTTTTTGTCTGTAACACCCCTTTCTGT ATTGCAGGTTGGCTTCTGAAATCAAATGTGACCCTAAGTGACTCCTCACTGTTTTTAAGATAAGAAAGCATTAGCTC TGTGTTTTCCTAGCCCCTCCGTTCTTGTACTTAGACCCTTCACTCTGAACAGCAGGTATGCCAGCCTGTACTTCCTG GCATTATTACCCAGTGGCCCATAGCTCAGAAGTGACATTGTGTGGTGTGAACGCTGTCTCCGGAGCGAGATTCCTAA CTCCTCTACTTTACAGCTATAAAGTGGACAAATCACTTCACTCCATGCCTCAGTTTCCCCAGGTATCACACATGGGG ACTATAATAGCATCTAACCACATAGATTGTTGGACAATTAAATGAGCTGACCAGCAGGAAACTCTTAGAACAACGCC ACCCCATAGTAAGCACTCAATAAATGGCAACTGCTGCCGTTGATGAGGATGACGATGGTGCTATTTCTACCACTTCC TAAGGTCGCCCCTGCGTGCTCAGCAGCCACCTCCACCCTGCCCTACCCGCTGCCCACATATTTCATAGGCCTCCTTA GATGTCCCTGTCTTTGAGCCGGGAGCCCTCGCACTCCCAGGCTTGCTTCAGATCCCTTGCCTGAGCCTGTCCAGCAC TTTGAGCCCGCTCTGCCCGAGCGCTGTTCTCCTTCCGTGCCGTGCTGTCTGCTGTGAGTCCTGGAGAACAGGGACCA TGTCTATCTTGCTGGCCTTAGTACCCCTCACAGGGCCTAGCCCAGGGGGCACTCAAGAAATTGTAAAGATCCATAGG CCGGGCGTGGTGGCTCACACCTGTAATCCCAGCACTTTGGGAGGCCAAGGCAAGAGACTCACTTGAGGCCAGGAGTT TGAGAACAGCCTGGGCAACATAGATTCTGTCTCTACAAAAAAACTTAAAAAAAAAAAATTATCCAGGCCTGGTGGAA GGCGTCTGCAGTGTCAGCTACTCAGGAGGCTGAGGCGGGAGGACCGCTTGAGCCCAGGAGTTCAGAGCTGCAGTGAG CTGTGATTATGCCACTGCACTGCAGCCTGGGCAACAGAGTGAGACCCTGTCTCTAAAAAAAAAATAAAATAAAAAAT AAAGATCCTGTAAGTGAAGGGTTAGAAGAGTGGAATATATGTGCTCTTAACAAAGAATGGTGTGATTATAGAGATGG GAGGTGAAAAGTAGCACGAATTCCAAGTTTTATTTTCGGCAACTCCGTCAGAGTCCTGAATGTTCTTTACCCAGTTT CTCACTCCCACTGGGACTCTCTAGGGCCAGCGGGAACCCTGCAGGCATTCGGAGAACTGAGCGCTGGCTGGACCTCC TCAAAACGGGGCACCCTCCAGGGAGCTGTCGAGGGGCTCTTCTGCCTCTTCAGAAACCATCATCAGCTGTGAAATGT GGGCTTCCAAGTGGTTTTGTGTCTCCTCAGTTTACAGAGTCCATGTTTTTGGTGACTCTTGCCTTGGACCCTGATGG CGCTGGGGGGTTTCATCGCCATTTAACCCTCCGCGTCTCACAGAGACCAAGCAGTGCTTTCGGGATCGCTCTGTTGG TCTCTGTGTAGCACGTGGCCCCCAAAATTTAGGTGTTGAAGTCCTCATCCTCAGAACCTCTGGATGTGACAGTATTT TTGAGATAGGGTCTCTTAGAAAGGTGATTAGGGTAACTCAGGGTCATTAGAGGAGCCCCTCACCCAGTGTGACCGGT GTCCCCATGAGATGGAGGTTGTGGCACAGACACACGGAGGGATGGCCACGTGAGGACCCTGGGAGAAGGCTGCCTTC TGCAAGCCAAGGAGAGAGCGCCTTGGAGAAAACCAGCTGTGCCTACACCTTGGGTTCAGACTTGCAGCACCCAGGCT GCAGGGCTTCGTCAGGGCAGCCCTGGCGGAAGAACGCAGCCTCGTCGGTGGGTTTGGTGATGCTGTGCAGTTCAGCT TTACCGTCTTGCTGCAGCGGGAACGTCCTGCTGTAGAAGAGGCGCCGCCATCCCAGACAGTGTTCATAAACACCGAC GGCAAAGTCAGGACGGGCAGGAGCGTTCTAGGCAAATGATGAAAATGGTATTTTACACATATTTGAAAAGAAAGGTT TGAGAAGGCATCCATTATTAAAACCAAATTCAACAGAGAATTTAAAAACTGGCTGTGGTGAGCAATGTTAGTCCCAA ATCACTTTTTCACAACTTGTTTCTGTTCTGTCGTAGGGCTAGTGAGACCAGCTTTCCCTTGAGTTCTGGGGGGGGAC GTGGAATGTTTGTGAGGCTACCCTTCATTGAAGGGGGGCCCGTGATGTTGAGTGTTCACCGTCTTTCAGAACGTTTG TGCGGTGGCGTGTCCCTTCTGTAAATTGCTGTGACCTGGGCTGCATGCTCCACAGTCAGACTCGCTTCGCCCTCTTA CTGGTCTTCATTAATTCGTGGTCCCTGGAGTGGAGCGGGAAATACAGATATCCACCTCCTCTCATTATCCAAATACA ATAACAAATTACGAAGCATCTTATCCTACCAACCATTATCCTATCAGAAAGCATGTGCTAATTTCGAGGAAGCAAGA GGAACTATGTCAGATGTGCCATGAGATGTAAAATGTACTCGCCCTGTATCTTGGTCTTCTCCTAGCAGTATCTTGTC TGACAATCTCCTTGTTTAACTTCTTGGGCTTTTTCTTTGACATCTTCCCACCTTTCCCTAAGGGTTTAACTGTCTTA GATCTCCTTGTCTGCTTCTGCCCGAGACTCTGGGTCCCTGTCAACTCGGGTAGAATTTTATTGGTATTTTTAGGCTC TTTAACATACTTTAATCATTGTGTTGCTGCATATATTTCTTCTAAGATAGAAGTAAATTGTAATTGTCCTCAGATAA TAACCTAGGACCTTAAAATACACACAAATGTACACACTAATGAATTTTGTAAATATTACATAACTCGGTAATTATTG TTTCAAATTAGATAATGCAGAAATCTCTGGATTTTTATGTCGGTGTTTATAAAGCATTTTAATGATAATTGGGGAAG TTGGTTGTGGGTTTTTAGCTAGAACAGGAACCCATTATAATTTTTCCCAATTATAATAATGGGACATAAGCCTCTGT AACCTGAAATTTTCCTCTATGACATGTTTTCAGAAGTAGAATTATTAGATTTGATGAGTGTGGATGCCTAGGTTTGT AGTCAACTTTTTGAACTGAGCTAATCAATGCATATTTGGGAAGAGCATATAATCCAAAATATTGGGTCTACAAATGT AAGATTGCCTTTGGTGATGAGTTGGTGTCATCTGTCTTCTGTAGGAAATGAAGGGCCTCTTGCAGTGTGCTGTGCCT CATTTTAAACCTTTAACTGGCAAGTGGTCCTGATCTTCCCTCTGGACAGCTGCGGGAAGAGGTTTCCCGGTTTACCT TGGCCCCCGTAACACCACATCATCCCACGTGTGCGTGAAGAGCCCGCACAGGCCCCCTGTGCGCCCGGGGTCAGGTG TGGATGGGGTCGGGGTATTTCCAGTACTTGCGCTTCGGAGTCTTTTTAAAATTGAGAGCTCATCTATTGTTGATAAA TGGCCAAATATAAATGTATAACACCTTGATTAATAGGCTGCTATGAAATTGAAGGTGGTCTTTTCTAGGAAATCCTC ATTGAAATACTGCCCATTTTGAATAAGTCTAATAGTACGATGAACCACACCCTTAGTGGAAACGTTATAGGATGCAA ATAGGAAGATTTGTGGGTGTTTTTCCTTCATGAATTTGTTGTTATCTTTGCAGAAGTGAAGAATATCAATATTTTAT GGCCTTTCTTTGGGATTCGTGTTTATTACCAGTGATGCAGAGCGTGGCCCTGGTCCTCAGCGCCTCTGCCTTTGTGG TGGCGGGGGCCAGTTTGCCTGGTGGGGAGGTGTGTGTGTTTTCACCAAGTTAGCAAATTTCCTGTTTTCTCTTTTAA AATGGAAGGCTAGCTTCCTCTCCCATTGGCTTTTTCTGGAGATTGGAAATGGTATTACAGTCTTTGTTGTGTTATTG TGGAAATCTCCGGCGTCTCTCCTGATGTTGCCGCCCTGCACGGCCGTTGGAGAGTACTTGCAGGCAGCCCCGCTCCA CACCACTTTTATCATCTTCATTTTCATTCTTTTGTGAAAATGTAGTTGCTTTGGAGCCTTTCGAAAAGTGTGTCAGA TCATTTTTGCCATTTCTCTTTTTCCTTTTACTTTCTGTTATTTTCCATTTCGATCTCAGATACCCTTCTTAGGAAGC TGCATCCCCGACGCCGGGCTGGTCTGTTCTGCTCTGGGGGCGGCGTGTGCTCTCATTTCACTCCCATAATCAGCTTT GGCATATGGGATTCAGAGACGCAGTGCTTGGTCTTCTGAGATGTTTTGTGCAGCAGCTAGGTGATGTCCCAGTTCTC CTGGAATATTTGCATCGACCAGGATGCTGTTTCTGCGTCGGAACGTGGTGTCTTTGTTTACCCTTAGGCATTATCTT GCTTGTTTACCTAAATCTTTAACTCTCCTTAGGTGAGTGACTTCACTGATACTAAGAGTGATTAAAATAATGTGATG TATGGAGTCAACTTAGGCAATACATTTACCTGTACACCACACGTCCAATATTTATTCAGAATGATTTTTTTCAAATA TTTAATAAGAATGAATAATTGAATGTACTATAGCCTTCTTACTTTGCCCTGCGATCCCATAAGACCTTTAAAGCTAA AACTAGTTTGGGCCCTTGAGGGGTTTATAGAAATTAGGTATGTCTGAGTATGGAAATGGCTGGCATGGTGAAAGAAT TGAGTCCTTAAGTAATTTGCTATTCCCAGTGATAATTAGAAGAAACCCCTGTTCTAGCTCCATTTTCCCATTTTTTT TAATGTTTTAGGGGAGATTTCTTTAGGTATTAGCGGCTGATCTTTTTTTTCTTACCATGTACTCCCGCTGTAAGGTG AGTGGTTTTGGCATATTGTGTTGCCTTGATGGGTTATTAAATCGTCACTTGGAAGGATGAATGTGGGTTATAGAGTA TGTTGTGCCATATATCATCTTAGTCATCAGGAATGTTTCAGAAAACTGAATAAGAGTAAAGTTTTTTAAATTTATGA TTGAAATTAGCAATTTTATTAGGTCCTTTTAAAAAAACCTACAAACTTTTAAAGTTTTTCTCAAAATATACATCTTT GTTCTCAGAAGAGGTCTGCAGACTGCCCTAATTTGTCTTTTGAAAGGACACAGAATTTTAGCAGAATACTTATGCTG CAGAAATGGACAAATGCAGGTGCTGTTCCTTGGATGGTCAGCATGGAAGGTTGACGTGCTCCAAGATTTACTGTGCG TCACCTTAGCCATCAGTCCCATCGTATTCATTTAACTCCACTAATAAGAAACGGAGCAAAAGTGGAATTATGAGTTC AGTAGTATTCTATGAAAGGTCAGTTGTTAAAAAGGCAGAGTCTGACATTCTGTGAAAAAGGTGCCTTCATTAATCAT TCTGATGGGAGAGCACGGGGCGTTCTTGACCAATACCAGTGAACCTTGCAATGCAAAGTTTCAGGGTCTGATGTGTA AGTTCTTATATAAAAACGAGGATCGTTTGAATATATGTTGAGATCATTCAAGGAATTGCTTTACCTTTGCTGTTTAA AAGTGTGTATATGATATAGAACACGTTATTAAATGTGCATTACTGTTTTGAAAAAGGAAAAACACCTGTTAATTTTA TAGATAAATTATGGAAATCTGTGGTAGACACAGCTCCAGGCAGTAATGCATTTCCAAGCGAATGAATGGACATTTCC TATATGGATCATCTACAGATATTGGTAAGGTTTCTTTAAGTCTCTTTTGTACTCTGCATTCGTGGCTAAACCTATGC ACTTTGACCCTGTTCTGCAAAACAAACCATGGCCTACTTTCAGACTCTCCTTCGTTCTTTTGCTGTTGGTTACGGTG GCAGAGTGCCTACTGTGTACACAGAATCTAGACATTTCACATTGAATTATCTGAGTATTTATGTGCCCTGACAATTC AGCGTTAAAGTTCAATACAAATATATAAATTATTAAGCAGTTAACCAGTGACACATTCATCAGCTGAACGTCTTTTA TTGTGGCCTGTAGACAGATTATTTTCATTCAGCCATTAAAGCACAATATTGTCGGGTGAAATTCTGTTTCTGGATTT TCTTTGTCTCTGTTGCTGCTATGGAATTTCATTGAAAGCATCGAAATATGAGACTAACACAGAATACTGAGTGTTTC TTTTCAGAAACGGGAACTTTCAGACATTTTCTCTCCAGCTGAATTCAGTAAAGAGCAGTGTGTGTGTGTGTGTGTGT GTGTGTGTGTGTGTGTGTGTGTGTTCCTTTCCCAGCCAGCAGTGTTATATCTGAAGGGAAGGGTGGGGATGAGCTGT GGTGCCGCCCTTCACGCCATTGAGACGGGCTGGGAAGGTGCCCTTTCACCCTCTACCTGCAGTTGGTGCCTAGTGAT TTTAAAGATTCAGGAATGTCTTTCACCGTGATTCTCACTATTAACCAATCTCACGTATCTAACTTTCCTTACTATAT TATTAGTTACTACTTTCTATACAAGAGAGAGGTGGAGCCTGCCTGTGTCACGATGGGGAAGGCCCTTGGGATTCGCT CTGTGGGTGCGGGCAGCGACAGGGGTGGGTCAGGGTGCAGGCGGTGAGACGGCGAGCGAGGAGGTCGGGAAGGAGCC TCCACACCCCAGCAGGGCCCATTCCACACGAGACCCCAGACGAGGGGAGCAGAAGCTGCTGGTGCCTCTCTGTTCTT TGCCGGATGCTACCGTTGTCTGTGGCCACACTTGCTTCTTCAGGTGTGGAAATAGAAGCTCAGAGAAGTTAAACAAT GCGCCAAATGTTACACCTTCCCATTCTAACGATTTTGTCAATTTCGGTGTCAAGGTGATATCTTGTCTGATGGTGGA GGTCATAGTTGTCTGTGAGGACATGGTATCCACCCTCAAGCATGTAATTATGCATGGAGAGGGCTCAGTTCAGTTTT ACCGAGACACAGTTTATTGGAGTCTACAAATAGCATAAACTGCTATGTCTGCAGTGTTACCATTCTCTTTCCTTATT GTCATAATTTTTGTTTAATGGCTGTTTGTGCTACATTGACCAGGTCCATGGGTTGCTTAACATAAATGATTACATTT GCTTATGATTAAAAACTTCCTCCATTAGAAGCACTGCACCCAGGACTTGTAAATGATTTGTTGTTAAAGTTTTCTAC TTCATTAGCCACGTCGTTGACTCCAGAGTCATTGTCTAAGAGTTAGGAACACTTGTAATGAGCTGTATAATATGGAC CCTCGAACGAAATTTAAAAAGAGACCAAGCCTTCTAGGAACAGCTTAGCATGCAGATAAAAGGCTAGGAAAATGTGT GAACAGCGCCAGGCTGTAGGCTGAGGCACGTTCCATTCTGACTCAGAATGGCATGGGTGAATAAAGTCTTTTATGAT GGAGCAAGACTTGTTGTAAAAAATTATATACCATTCATGACCAAAATCCTTCAAAACACTCCGTCACCATTCCCTTC TGAACCCTATAGCTTAAGTTCTAAGCATCTTGTGAGTCTCTTGATAGAGCGTTGAAAACTCCATGCAATGGTCTTTG GGACGCCATTCGAACCTGGCCTGCCCCTTGCTGGCACCTTGTTCGTCTTTGGCTCCATCCGGGGATTTGATTGTCAG TGACCCACCTGATTACAGTGGGCTGCCATGGCCCTGGCCTCGTGGTACAAAAACATGGACGATTTTGTGATTTATTA TCGGAGGACAGCTAGCAAAGAGAACTTCCTGGAATAACTGCATTAAATGTGATGGGCATTTTTGTGACAGATGTAAA ATTCTTAATCTTGAAAGCAGTTGCATGTGTCAGGCAGACTGGGTGTTAATTTTCTGACAGCAGTAGGAGGAGCTTTT TTTCCCCATTACAGATGGAATTAGGTCTACTTTGTACACAGTACTTTGTGACAGTGACTCTGGCCAGGGGAGGGGAT ATGCTGGTTCTGGAGTCTGAGAAGATGGGGCTGAGCCAGCTGTGACTCGGCCACACGTGGCTGTGGGACTTCGCACT GACTACGCAGCCTCTGCCAGGTTTCTTTGTAAAAGTGAAGTTAACACCGATCTCACAATATCTGGGGAGGAATAAAT AGGAACACACATGCTGAGTTAAAGTCCTCAGTTATTTCGTCTTCAGCGACAGATGATCCCCGACTTACCGTGGTTCA GCGTTTGAGTTTTCGAGCTTTATGATGGTGCAGAAGCAGCCTACGTGCAGTAGAAACTGTACCACAAGTGCCCGTGC CACCATTCTGCGTCTCAGTACAGTATTGAATAAAGGACATGACACAGTCACCACTTTATTATAAAATAGGCTCGTGT TCACTGATGCTGCCCAACTGTAGCTAATTCAAGTGTTGTGAGCACGTTTAAGGCAGGCTGGGTGGAACCATGGCGTT TGGTGTATTGAATGTGTTTTCAGGGTCGATGTTTTTAACTTTCACTGAATGTATCAGGATGTAGCCCCATCATAAGT CGAGGCACATCTCTGTTTGAGTGCCTGTTACGTGCTGGCCATGGTCCCAGGCCTGCGGGATGCAGAGGCGAGGACAG TGTTTGAGTTTCTAGCGTGCTCACTGTGTGTGGGTAGGATGACCCTGCCGGTCTGTGCCCTGGCTTAGTGAACTAGA TCTCGTTGGTGTCCTGAGGCCGAGTCTTCTGTGGTTTCTCTGGAGAGTGTGGCTCTCCCTGGTCAGTATTGTGACCT CAGTTACCACTTTCTGTCCCCTACATTCATATACTCCCATTCATGTATTTAACAATTCTTTACTGCTGTAAGATAAA TGGTTTTCAATTATGAGTTTTGTCATGAAAATTTAATTTTTTAAAAAAAATCAAAAGACCTGGATGTGGTGTGAGCA TGCGGAGCTCCCCTTTCTGCCCAGGCCTTTACCTTCCTGCTAGGAGGCAAGGTCAGCCCAGGGACAGTGCTGGGAGA GAGCGGAGTTCCGTCGTTCATGGGTTGCTAATTTTTGGAGTACTGCAATACAATTAAATATACTTAACCTTAAAATT ACCCAGAATGTGATTGAATCTGTGATTCACAGGCCCCTTCTGGTTCTCCCGTGCCTCGGTGTGATCATTAGGAGGAT GGATCAACCTGGTGCTATCGGGAACGCACTGCGTTAATGGCCTCAGTGCCCAGCAATGGAGAACTTTCCACTGAGTA TCCGCATGCAGCGTGCCCGGCGGAAGGTGAAAGCTTCACCCTTTGTCTCCCTTGCCATCCTTTGGCCTTGTGTGTTT GGAAGTAGCCAGAGGTGGTGGAACAGTGGTGGGCTGGTTTGGGTCTTGTTGGCAGTGCCAAAGGGACAAAGGCTGGA TGTGGGCACGACCTCAGCCTCGTGATGGGCAAGACAGGTGGGGGTGACTTTCTGTTCTGAAAATGACCTCCTCCCCT TCTTCCTCCCCTCAAGCTTCCTACCTTTCCTCCCCCTCCCCCTCCCTTCTTCTTCCACTCCCCCACTTCTTCCTCCT CTCCCCCTCCTTCCCCCTTCCCCCTCCTCTTCCCCCTTCCCCCTCCTCTTCCCCTTCCTCCTCCTCTTCCCCCTCTC CTTCCTCCCCCTCCTTCCTCCCCCTCTCCTTCCTCCCCCCTCTTTCCTCCCCCTCTTTCCTCTTCTCCCCTTCCTCC TCCTCCTCTTCCTTCCTCTCCCCTCCCCCTCCTCCTCCTTCTCTTCCTCCTCTACCCCTTGCTCCTTCTCTTCCCCT TCTTCTTCCTCTCTCTCCCCTTCCTGTCTTTCCCCTTCCTCCTCCCTTCCTCCTCCCCTCCTCCTCCCCTTCTTCCT CCACTTCCTCATTTTCCTCCTCCACTTCCTCATCTTCCTCTTCTTCCTCCTCATTATTTTCCTCCTCCTCCCCCTCA TTTTCCTCCTCCTCCCCCTCCTCCTCCTCCCCTGCTTCCCCTCCTCTGCTTCCTCACCCTCCTCTCCTTCCTCCCCC TCCTCTCCTTCCTCCTCCTCCTCTCCTTCCTCCTCTTCCTCCTCCTCCTGCCCTTCTTTTCCTCTGTCTTCCTGTTT TCTTCCCTTTCCCCTGCTGTATTCCTTCCTCACACGCCCCCCTCAGCCTCTTTTCTGGGGACTGGCTCTTCTAGGAG GCCTGCCTCAAGGCCCTCAGGCCAGTTCCCGGGGCAGCCACTGAGCAGAACCGCCCCTCCCAGCGCCCACCTATGGC CACTGCCCTCAGGGTCCCTTCCTCCAGCATGGTGCTGGCGGATGGCGCCATAAACAGGCTGTCACTCATCTGCAGGC TGCCTTGTCTTTCCCCCAGCAGATTAAAGCCTTCATGAAAAAAGATGGAGGGCGCTTGACTTTGCGACACGCACATT CTGCCGTCTTGGGAAGGGGGAAAGAGGGGCATCTAGCTGGGCGCTGCGTGTAGCCTGCCCGGCGGGGCCGGGGTGTG GCAGGACCGAAGCCTGCTCCTCGTGTGGGGCCCGGCGCGAAAACAGCTTGTGCAGCCGCTGAGTGACTGAGGACCGT GTCCCAGTGTAGTGTCATTTCCTCGTACATGTGGAGTTTAAAGAATTATAGGTACCTTGAGCTACTGGCAGGTAAAA GACAGTAAAAAAAAAAAAAATAGTGAAAGACACAGTCTACCCTCTGAACTGATCTACAAGGCTATGAAAGCATCACT TTGAATAATATGCAGTAGATATGTTAGAAAAATGTAATATTGGTGTGTGGACCCCTCAGCCTCTTTGACGAGTGTTG TAATATATCTTGTTGAATGATGAATATGCTACCGTATGTTTCAACTATTCCTTTCTTGTGGCTGAAAGAAAAATATA TTGAAAATGAATGTAAAATATTGACATTTAGTAGTTGTTATGCTCCACTGTAATTTCTTGTTGCTTTGGATGTGGTA GACACCTCACTAATTACACGGTGTGTACATGCGCACCTGTAACAGGAGCCGCGTGAAAGTCTGATTGGAACTCTGAC TTGATCAGCCCATGTTCTTCTGCCATAGCCACAGGAGCATCTGAACGTGGTGATGGAAGAAGACCCTGGCGCCCCGT CTCCCCGTGGATTAGCGGCCCTACTGTGAGATCCTGGTGATCTTGATGATCGAGGGTTATTTGAAGCAGTTCATTTT GTGCTGCAGTTCATTGAAATGCCACCACCCAGAAGACCCCTGAGTTTCCAGAGGTCCTGGTGCTTTTTTTGCTGGGG CTTGGACCGGAGCGTCTTTAGAGTGACCACTGCCAGGGAAGACACCGTGGTCGGGGCCTGTGATTCCTGCCGGGGCC CCAGGCAGGCTGGAGCCACAGGTCTGAGTTCTGTCCAGTTCAGGTTCGTAGGCCACTCTTGCAGCTCTGTTCCTTCT TCAGCTCCTCTGGTCATTCACACGTGGCCTGGCCACTTGCCTATTGTACCAGCTCTCCAGGGCTGGGGACTTGCTAG AGCAGGGTTCCCAGTGCCCCCAGGCTCTACTTTGCTCTGCCTGGTCTCAGGGTGTAGGGGATGGAGAGCTGGACTTC CAGCCTGCTTCTTGGCTGTCTAGGTGCCAGGGGCTCGGGACACAGAGCTCCTGGAGGCCGAGCACAAGCCTTGGGCA GAGGTGAGGCAGAGCTCTGACTGTTTCATTCGACTACGTTGCCAAGGAGATGCTCGCTCGGAGTGGTTGCTCTGGCT CTGGGATTCCAAACCAAGCTGCCTTCTCTGATGTGGCCTTAGTGCTCTGGGCGGATGTACCTTGGCTCTGCCTGGAC CCTCTCTCTCTTCCAGGCCTCTGTCCCACCAGGATGATGCCTATCCAGAGCTCATTGTCCTCTCCCACTTCCTCCCC GAGCTTCCCATTCCGTGTCTCTCTGGAGGGCCCATCATCATCCTGGTGGAGGTGTTGCACTGAGGACCACAGCAGCC CTCGCATTCCTACACATACCCCTTTGCCCGTGGGATTTTTGCTGTTTCTCTTCTCCCACTGTTGGTCGTTGGCTGGA TCTCGTTGAAGGCCTGCGGAGATTCAGGTGCCATAGGGAGGGTCTGGGACTTTTCTCTCGGAGGCCCCATGAAGTTG GTATTCCTCAGCTCTGCCCCAGGGTCCCTCTGATTCCACGGAGGAGCTGCGGAAAGTCCACATTTTTGAAGAGCTGC AGGCTGGGGAGCCCCCGTGGCGAACAGAGGCTCTAAGAGAGGGGTCTTTGTTTTGGGGCAGTGAGACTGAGTGGCCA GTGAGCAATCCGTCATCAATGGAGATCAATACCCAGGAGGTGCCATCTCTCTTGGCGGGTGACACGTAACTGTTACA AATGGGTGCCTGTCCCTGTCACGTCACCGGAGTGTGCGTCTTGATGCCCTGGCTCCCTGAGGCAGCTCGTGTTTTCG AGTGTTTCCATTTTAGAGACCCGGAAAAGGGATCCTTAGAGCTGCTTTGTTTTCCTTTTTCACCAGAAACAAATGCC TTTGTGCTTCTCCCGCCTGCGGACTCCCACTGCGTTCAGCCCGTGCGGGTGGTCATCGGCCAGACCCATCCAGCCTG TGGCCCGTCCCCTCCACCTCTCACAGCCACCTAGGAAAGTAGTTCTCAGCTCTAAGGGGCATTGTCTGTTCCAAGAA GAATGACTGCTCCCTGCCACCTCCCACCCCCATCCCATGTCCCCATCATAGACCCAGCAGGACAGACCCCCCCAGAG AGCCAGTGGCCTTCTTGCCCATAACTCGTTGGTGCTGGAGAGCTGTGGGTAGCGTAGCAAGTGCCACTGAGCATCCT GGAAACCCTGGGAGGGGTCCCGGCGAGAAAGAGCCATCACCCTAAGAAGATTCAGGAATGGGATGATCATGGGGTTC AGGGTGCCCCAACAGTAGGTTGAAAATCCTGTCCCTGGCCAGGAGGCAGGGAAGTAGCAACCTTATTCAAGGGGAAA AAAAACCTCCTGGAGTCTAGATACAGTATCTTAGTAGTAATAAAAGCACGGGTACAGATTTACTGGTGTCGGAGTTT TAACTGTAATACTGTGGAATGGTAGGTTATTTTGTGTTCAAGAGCTACATTTATCACAAAAATGATGATATACAAAT CACTTCCATATCACAAAATGTTCTTGGTCAAAATTGGCTTGAGGCAAGTGATATCTTCGAGTAAACAGTATATGAAG ACCATATCTTAAAATGATAGATCACCTAATTTTGTTCTGACAGAGGTTTTTGCCGCAACAAACATCTGTCATTGAAA TACCAATTCAGAGATATGGAAGCCAAATTGTAGAGGTTTTCCTGGATTTGTCACTCCTAACTTAAGAATGCTACTGT TCCCATCGTAGCTTTTATAAGATATTAGGCAGTGTTGCACTTTAAGAGACATTAAAACAAAATAAACAATTCAGAGA AATACGCTTTAAATGCAAACCAACCATTAAAAAGTATAGAAGTCAGGAATGGGCCGAGACAGCCTGGCTGTTTTTAT GTGCAGACATCACATTAAGCTCGCTTTACTTGGAGATTTTGAACTGTAGAGGCCTCCAGGCCTCCTGCCTTCACTTG AGGTTTTTGGTGCTTTTTAAAGGACTGTGATAATGATGTTATTCCCAAGATGTGAAGGACCATTCTGGTGCTGTCCT GACAGGGGAGCAGGTTTACAGCTCAGGACACTGGGGTGCCACGGGGACCTGGGGGTCTCCTTGTGCCTGGGATTCCT CCTCGTCAGGAGCCCTCACTCAGGAAGCCCTGCACGGGGGCAAATGTCCTGGGATTGGAGAGGCAAGCGTGAATCAG ACATTTCCAAAGACGACACCGAAGAACTTCTGCTGTATTCCAAGTTAAAAACTAATTGATCTTGGTCCAAACTATCA TAGGATAGTTAAAAAAAACACACAATAGAATTTCTGAGGTGCTTGGGAGACATTTTAGAAATATGTCATTGGTGACC ACTTTTTATAGGCTGAATTAAGAGGAAAAAAAGGTAAAATATCTTTTAAAGTCTCCAGTTTCTGAGACCCCTCTGGA GATGATTTCATTGAGTTCCTCCCTCTGACAGCTCCTATGGACCTGGAGGGCTCTGGTTTCTGGAGTAGCATGGGAGC TGGCCCTGGTCCTGGCTCTCCTGCCCACCTCTGCCCCCTTTTCCCTGCCCCCTCCTGACTGCAGCCACCAGTGCCCG CCATTTCTTCCTGCGCTGCAACAGCTCTGGCCAGTCCTCTCCTTATTCTGTAGGTTGAAGTACATTCCTGGAATCCA GGTTTTCAAAGCATTCTATGCAGTTTCTTGCATGAAATACAGGAGCAGCCTTTCCTGGGCTGCCTCACACATTGGGC TTCTGTATTCCTTTATGACAGTGTGAGGCAGGCCTGGCTAATGTCTTCCAGCCCATTCATCTCCCCACAGGGTATGA GATGATGGGCTTTAGCGTCAGACAGCTGGGCCCACACTCCGGCTCGGCCACTTACGGGCTCTGGCAGGTTACCTCAG CTGTTGAACTCGTGAGCTGTGACTTCCTCCTCTGCTAATGCGGCACTCTCATGCCTGCCTCGCAGGTTCAGATGTGA CGATAGACATAAAACACCTGGCCCGGCTCCGGCGCACAGATGCTGAAGTCAGTTTTCTGAGGTCGTGGTGGGGGCGG GGGTGAGGGTGGCAATGGAAATAGTGACAACAGTAGTTGGCGTTGTCATCCTGTTTACGGCTAGCCTGCGAGTGGCT CAGGCGAATCCAGCACAGCCTTGTCCTCGGGTTAGCTGCCCTCTCAGAGCTTCACATCATAACTAGTTCATTTATCA TCCACGCTAGAGACTGGTCTTCCAGAGGATATTCCTACCTCAGAGCCCCTTCCTCCCGACGTGCTGGGACGGCCCCT GCTGTTGCCTGGCTTTGCCTGGTATTACAAGGGACCTCAACCTCCTGTGCTCTTACCCGGGCCTGACTTGGGTGATG CCCAGAATCCCAGTCATGAGGGGACCATGACTGTGGGCCAGTTGCTGACAACCTTGGTGGGTTGTAGACTCATGAAA GAAACACTCCTTTGCCTCCTGACCTTGTGCATCAGGGAGGATGCAGGAGGAACGGAAGATTGAGCCCGGGGAGTGCT GTCTCGGCAGTGCCACTTCAGACAAGTGAGTGTCCCTTTGTGGGACGTGTTCTCAACAGGCACAGGTGACCTTATAG GTATTGGGATGATGGGGGTGTGCCTGCAGGTTGTGAATTACTTTTTGCCTTCAGAGCCACCAACACATTCACACAAA CATTTGTTTTCCTTTTGATTTTATCAGGAGAATTTTGTTTCTTTGCCCGCCTGAACATTGCTTGCCCTCCATAATTG AGTGTAGACTAACTCTGCAGCCTAAGCCAAAGTACATCTTTTGTCTGTGCGAAGAGAAAGTTCCATTTCCCTTTACC AAGTTATCTCCATGGCCCTTGGCCACAGCACCCTCCGCAGACCCTCACGTGCATGGCCGCAGCAGACTGCCCACACA TTTGCACACTGACATTGGTCTTATTCTCATCAACTAATAATTAAACCAGAATCCAAGTAAGTGCAAGAATCAAAGGC TTTGAGTAGGAACAAAAGGCTATGTTACTTTGTATCCTTAACTAAGAATTTTGTTGTTCAAGAAATCCTGGGCTTTT GTTGGTGTAGCTCTGCAAGTCAGCAGCACCCGGGTTCCACCCGGTGCTTACAGGTCACGAGTCCAGTGACGCTGATT GAGGTGTGTCACCAAATATGCTGCAGATCCGGGGCCAGCCCACCTGGGCCTCCGGCCACGGTCTGTGCCCACGTGTC ACGAGGCACATCCCGGGTCATCCGGAGAGGGACCTACAAAGTCTATTTTCAAACCAAGTTATTTTGTGAAATATTGT AGTGAACTTATCAAAACTGGTGAAGTTGATGAAGTGAAATGAAAATACTGCCATGAAAATTATTTTAGTGCAACATA ATGAGTTCTGTTTTTCATATCTGGCAGATATTACCTACTAAATCTAATCTGCAATGCTGAGAATTATGCCATTTTTT ATGATTTGTTTAATTTTGACTGAAAAAAATTGACTATTCATCGATTCTTGACCTGCGAGTATTGAGGGAAAAATTTA GTTAATAAGAATTTTCATGAGGTGTAAATTTAAATGTATGAGGATTCTGCCTATCCTTTAATACTATAGACATTAAT TTAAAAATTCTATAAGGGCCTGAGGGCCTGGGGTGTCTGAAGAACAGAACATGGAGGCAGTTATGGTTGTTAACTGG TAGACAAGGTCTGTCAAGATAATATAGGAATATTTTGGTGTTTTCCCTATATCCTTGGTTATATGTGGCTGAGCATA ATTAGAAGTTAAATTTGTTTTAGATAATCTCTTCGGCTGTGAAATGGCATCAATACACTTAAGAGTAACAAGTCGAA TAGAAATCAGGGCCTGTTGTTAGTGTATGAGCACACACCACACAAAGTTTCTTCTTGGTTCCCTATTTCCGCAGTTC AAAGGGGGTTTTAAAGAAGTCATAAAAAATGAAGGATCCTAATATTGCAGTTGGAGACTGAGAGTGAATGACATATT CTTTACACACCTCAACTGTCAACATTTCTAATCAAATCACCCAGCCTTCTTACTTCTTAAATGATACAGGCGATATT TCCTGTAATAAACACCCCTAAGTCTAATGAGAGGCCCTAAGGCAACTTGAAATATATGAATTCATTATAATTGAACA ACATAATCTGCAGGGCAAAGATGCCTGCTACCCAGCTCTGAACTTCACAGAGCTTGGGTGTTTTATGAAAAATGTTA TACGTTTTGTTTACAACGTATGGCTGCTGTTTGAAGTATTGTATGTTCCTGTAGTACATAAGACGGGGCCGCATAAT GGAGGAAAAATCAAGGAGGCAATCTTTCATTTTGTTCTGGTATGAAAAATTCAAAAGACTGAAAACTCACCCAGAGA AATTTTGCAAGCATATTATTTTCCAATGTTTCGATCAATTTGTCTGTCAACCATGCTGAGAGGTGGAAGGGGCCAGC AAAAGCAATTTAGCTTGTGAGGTCCAAAATAGAAATGTTTTAGGGAACCTTGTCACATGTTATTTCTTTATATTAGA ACCGCCGTGTGTTTTATTTGCTGGTCATAATATGAAGAGAAATTGATATCAATCATCTAAGTAAGCTATGAATATCT GATATAAATATGACTTTTATATGCATGCATCAGAATACAATATAAATTAACCTCTTCATGCATGCAAGAATCGTTAG TGCGACAGCGGGCTCCCTGTTTTGGATTTTTCCATTTGACAATTCACGGTGCATTTTAATTGATTGTACCCAAGTGA CACTCCGTGGAAGCATCTTGCATGTGAACGTAACTGACCCTGCCAGGGCTTTAATTAAGCTATAATCTTCCTTTAGT CAGTGCATCTGGGATTGATAAACATATGTAAACCATGCTGACCATCTAAGTGTTGCTTTAAAGAAAAAAATGGTGCA GATATGGGAGTTTTAAATAAACGGCTTTGATGTTACCTCCAGTTGAATTTTTAGTTGGGATTTGTTAAGATCAAGTT ATCTTAGGAGTAGTTAATTCTAAGGTGAATATACAATCTTGGTGAAACTTGTCCTGGAAAACCTGTAATTCTGTGAT TAGTCTGTGTGTACCCCAACACTTTTTCTTCAAATCTTAGATTAACACTGAATACATTAAGGTTATTTTTCAAACAG GTTATAGTTCAGAGAAATTAATTTAAATCAGTTGGCTTACAGAAGACCCAGTTTAGTTTTAATTTGCCTTAACAGCC AGGGTCTTCCTGCTCTACTTACTTAGACGTCCTCAGAGTGTCTTCTGAGTAGTTGGCTACCATTTGCATGGCATGAT TCCGCTCTCGGAGCTTGATATCTTTCCAGATTTTCAGAATTTAGAATTAAGTGTGGAAGTCAAGCATACTGAAAGTT TTTTATACTATGGATGCTGATAAATAGAAATAAACTATGACAAATTAATTCAGAAATCTTTCCACTCAGTGTAGATA TTAAGTTGTGGTCATACGGAAGAAGTCTTGAAACACGATTTACAGTAAATTATGATAATTTGGGATAGAGACTGTGC CATTAAGAGTTGTTAAACAGCATGATTCAGACAGCTCTGTTTTTTCCAGTGACTGCCTATTCCTTTATTGTATATGT GTACCCAAGTGATGCTCTCACATTTATCTTAAGTCCCTATTATTAATATACATTTAAAGTCTCCCGAGGTTTTGTTT GGTTTGCTTTGGTCCTTTTTTGGAGTGATTTGAAGCAAAGTTAATTGTGGAACCTATTTATAGACATCTTTCTATAC TTGCACAAGTCTGTGTATAATCAATTCTGAGAAGTAGAATTTAAAATTTAGTGCTTTGAAAATTGTCCTCATTTTAA TTTTTTGGATTTTAATTAGGTAAGGTTGAACATTTTTCCTTATTTTCATTGATTGTTTGGGGTTCCTTGGTAAAATG CTTGCTTTTGTTTCCTGTTGGGTTATTTTGTCTTTTTCTTATTGATATGTGAGAACTTTTTGCATATTAAAGAAAGA CCATCATTTCTATTTTGATTTTGCCCAGGGAATTTTTTTCAGAATAAGAAATTCAACTAGATTTTTAAAAAACTTTT AATTGGGCAATAATTTCAGATTTACAGAAAAGTCACAAAGATTGTACAGATCCTTCACCCAGGATCTCCCAATGTTA ACAGCTTACATAACCATAGTACATGGTTTGTCAAAACTAGGAGATTAACATTGGTCCATTGTGGTCAGATTTTTAAG TCCCTTCCTATGGCTTCTAAATTTATGAGGTGCTTACACAAAGGCCTTTTTAACCTTAAGATTTTTTTTTTGTTTTG CTTTTGTTAATCATATATGTTTATTTCCTTCTACTTTTTAAATGTTGTTTTTCATATTTTCATCTCTATTTATTTAT TTGAAATGTGTTTTAATTTAAAGAGTCTTGGTTGGAACCTATCTTTATTTTCATCTAAATGCTAACTAACCAACTTT TTCAGTATCTTTTGAATTACTCGACTTTGTGTTTACACACATTTTACAAGGACAACAAAAAAGTGCCTGCTGATTGA CTTTGTAGTCAATCATAACCCTTGACCATATTTTTTTTGGTGGTACTTCATATATGAAAGAGTATAAAATCTAGGTT ATTTAATGTTGTTATAAAATTATTCTAAGATCACCGAATGGAGAATACTTAGATTCTAAATAAGTGACTTTGTGAGA ATTGAATTTTTATATCATTAACCAATTCTAAGACATTTTAAAGAAGGCAGAATGATCTAACCCAATACTTCATATAC TTAGTTTTACTGTGGAATCCCAAGGGCAACATGTCCCCCGACCCAACGACAAAATAACATATATTCCTATTAAGTTT AGGGATTGATGGAGCTTGTAATTGTTAATTTGTCACAGATGATACTGTTATTTATGCGCAGTTACTGTCTCCCTTTG GAGAGTTTCATGTGCTAAATTTTCAGCAAATAGTTTGAGCCTCACCTACAGCTTCTTGACCTTTTGGGGAAAAAACT ATCACTTTAAAAGACCTTTTACGAGTGAGCAAATATATGCTTTTCTGTTTTCACAGTAACTTTCAGAGAAACCCACT GGTCTCAGCATGGCCTATGCCTTGGCAGCCCTTTCCACCCGTAGCAGGTTGTTCTTTACCTTTCGTTTTTAAGGTGG CTTCCAGTTCATACCAAGCTCACATACTGTGGCGAAGAGCTTTCTGAGAAACTTCATAAGATTTTGCTAAGATATTA ATGAATAATTTAGTATATTGTTGCATATATGACTGAGTATCACATTTCAGTAATGTTTTTACTATCTTTAAATATAG GTACATAAATCCTAGCTTGGGGTAAAAGGCATTGACATTTTCTCAGTAATAGTTCTTTGTAGACAGACCTGGGTTCA ATATTAGCTCTGCCACCCCTCGAGCTGTGTCACCAGAAGTAGGTTGTCCTGTACGTCTAACTTCAGTTTGTCTATTA AAGGGGATTGCACCACTGACGGCTTGATCAGGGTTAAATACTCCAGCATTCCTTGGAAAACACTTCTGTGAGCATTG TCGCAGACTTGATGATGTAGCAACTATTTTCCCTAAGTTTCCCACTTTAGAAATTATTAAAAATATTGCTGCCTTCA TAGCTTTTTCAAAGGTTTTCTCTTATTTCTGTCAGATCTCTTACATCTAATATTCTCATTCCCAGCAGATAATTAAT CTATTAAGAGGCAGGCTGTGGGCCCCTCTGATACCAGGGAAAGTGAAAGGCACCAGGGCAGGAGTTCTGATTCTAGC AACTGCCTGGCCGCTGTGTGATCTCCACACAGCATCAAACCTTGCAGAGTTCTAACAGAAGTGAATAGAAGATTAGT CTCCCACACAACAATAATGGGAGACTTTAACACCCCACCGTTAACATTAGACAGATCAACAAGACAGAAAGTTAACA AGGATACCCAGGAATTGAACTCAGCTCTGCACCAAGCGGACCTAATAGACATCTACAGAACTCTCCACCCCAAATCA ACAGAATATACTTTTTTTTCAGCACCACACCACACCTATTCCAAAATTGACCACATAGTTGGAAGTAAAGCACTCCT CAGCAAATGTAAAAGAACAGAAATTGTAACAAACTGTCTCTCAGACCACAGTGCAATCAAACTAGAACTCAGGATTA AGAATCTCACTCAAAACCGCTCAACTACATGGAAACTGAACAACCTGCTCCTGAATGACTACTGGGTACATAACGAA ATGAAGGCAGAAATAAAGATGTTCTTTGAAACCAACGAGAACAAAGACACAACATACCAGAATCTCTGGGACACATT CAAAGCAGTGTGTAGAGGGAAATTTATAGCACTAAATGCCCACAAGAGAAAGCAGGAAAGATCTAAAATTGACACCC TAACATCACAATTAAAAGAACTAGAGAAGCAAGAGCAAACACATTCAAAAGCTAGCAGAAAGCAAGAAATAACTAAG ATCAGAGCAGAACTGAAGGAAATAGAGACACAAAAAACCCTTCAAAAAATCAATGAATCCAGGAGCTGATTTTTTGA AAAGATCAACAAAATTGATAGACTGCTAGCAAGACTAATAAAGAAGAAAAGAGAGAAGAATCAAATAGACGCAATAA AAAATGACAAAGGGGATATCACCACCGATCCCACAGAAATACAAACTACCATCAGAGAATACTATAAACACCTCTAT GCAAATAAACTAGAAAATCTAGAAGAAATGGATAAATTCCTCGACACATACACTCTCCCAAGACTAAACCAGGAAGA AGTTGAATCTCTGAATAGACCAATAATAGGCTCTGAAATTGAGGCAATAATTAATAGCTTACCAACCAAAAAAAGTC CAGGACCATATGGATTCGCAGCCGAATTCTACCAGAGGTATAAGGAAGAGCTGGTACCATTCCTTCTGAAACTATTC CAATCAATAGACAAAGAGGGAATCCTCCCTAACTCATTTTATGAGGCCAGCATCATCCTGATACCAAAGCCTGGTAG AGACACAACCAAAAAAGAGAATTTTAGACCAATATCCTTGATGAACATTGATGCAAAAATCCTCAATAAAATACTGG CAAACCAAATCCAGCAGCACATCAAAAAGCTTATCCACCATGATCAAGTGGGCTTCATCCCTGGGATGCAAGGCTGG TTCAATATACGCAAATCAATAAATGTAATCCAGCATGTAAACAGAACCAAAGACAAAAACCACATGATTATCTCAAT AGATGCAGAAAAGACCTTTGACAAAATTCAACAACCCTTCATGCTAAAAACTCTCAATAAGTTAGGTATTGATGGGA CGTATCTCAAAATAATAAGTGCTATCTATGACAAACCCACAGCCAATATCATACTGAATGGACAAAAACTGGAAGCA TTCCCTTTGAAAACTGGCACAAGACAGGGATGCCCTCTTTCACCACTCCTATTCAACATAGTGTTGGAAGTTCTGGC CAGGGCAATCAGGCAGGAGAAGGGAATAAAGGGCATTCATTTAGGAAAAGAGGAAGTCAAATTGTCCCTGTTTGCAG ATGACGTGATTGTATATCTAGAAAACCCCATCGTCTCATCCCAAAATCTCCTTAAGCTGATAAGCAACTTCAGCAAA GTCTCAGGATACAAAATCAATGTGCAAAAATCACAAGCATTCTTATACACCAATAACAGGCAAACAAGAGCCAAATC ATGAGTGAACTCCCATTCACAGTTGCTTCAAAGAGAATAAAATACCTAGGAATCCAACTTACAAGGGATGTGAAGGA CCTCTTCAAGGAGAACTACAAACCACTGCTCAATGAAATAAAAGAGGATACAAACAAATGGAAGAACATTCCATGCT CATGGGTAGGAAGAATCAATATTGTGAAAATGGCCATACTGCCCAAGGTAATTTATAGATTCAATGCCATCCCCATC AAGCTACCAATGACTTTCTTCACAGAATTGGAAAAAGCTACTTTAAAGTTCGTATGGAACCAAAAAAGAGCCCACAT TGCCAAGTCAATCCTAAGCCAAAAGAACAAAGCTGGAGGCATCACGCTACCTGACTTCAAACTATACTACAAGGCTA CAGTAACCAAAACAGCATGGTACTGGTACCAAAACAGAGATATAGACCAATGGAACAGAACAGAGCCCTCAGAAATA ATGCCACATATCTACAACTATCTGATCATTGATAAACCTGACAAAAAGAAGAAATGGGGAAAGGATTCCTTATTTAA TAAATGGTGCTGGGAAAACTGGCTAGCCATATGTAGAAAGCTGAAACTGGATCCCTTCCTTACACCTTATACAAAAA TTAATTCAAGATGGATTAAAGACTTAAATGTTAGACCTAAAACCATAAAAAGCCTAGAAGAAAACCTAGGCAATACC ATTCAGGACATAGGCATGGGCAAGGACTTCATAACCAAAACACCAAAAGCAATGGCAACAAAAGCCAAAATTGACAA ATGGGATCTAATTAAACTAAAGAGCTTCTGCACAGCAAAAGAAACCACCATCAGAGTGAACAGGCAACCTACAGAAT AGGAGAAAATTTTCTCAACCTACTCCTCTGGCAAAGGGCTAATATCCAGAATCTACAATGAATTCAAAGAAATTTAC AAGAAAAAAACAACCCCATCAAAAAGTGGGCAAAGGATATGAACAGACACTTCTCAAAAGAAGACATTTATGCAGCC AAAAAACACATGAAAAAATGCTCACCATCACTGGCCATCAGAGAAATGCAACTCAAAACCACAATGAGATGCCATCT CACACCAGTTAGAATGGCAATCATTAAAAAGTCAGGAAACAACAGGTGCTGGAGAGGATGTGGAGAAATAGGAACAC TTTAATACTGTTGGTGGGACTGTAAAGTAGTTCAACCATTGTGGAAATCAGTGTGGCAATTCCTCAAGGATCTAGAA CTAGAAATACCATTTGACCCAGCCATCCTATTACTGAGTATATACCCAAAGGATTATAAATCATGCTGCTATAAAGA TACATGTACACGTATGTTTATTGCAGCACTATTCACAATAGCAAAAACTCGGAACCAACCCAAATGTCCAACAATGA TAGACTGGATTAAGAAAATGTGGCACATATACACCATGGAATACTATGCAGCCATAAAAAATGATGAGTTCATGTCA TTTGTAGGGACATGGATGAAGCTGGAAACCATCATTCTCAGCAAACTATTGCAAGGACAAAAAACCAAACACCGCAT GTTCTCACTCATAGGTGGGAATTGAACAATGAGAACACATGGACACAGGAAGGGGAACATCACACACTGGGGACTGT TGTGGGGTGGGAGGAGGGGGAGGGGTAGCATTAGGAGATATACCTAATGCTAAATGACAAGTTAATGGGTGCAGCAC ACCAACATGGCACATGTATATGTATGTAAGAAACCTGCACGTTGTGCACATGTACCCTAAAACTTAAAGTATAATAA TAAGAAAGAAACAAGATGAGCTTGTGGACTGTCTTTCACATCCTGGGAACTTATAGAAAGGTAGTTAGGATTATTAA GCAACTAAAACATTTTCCTTTATTTTTCCTTCCTTACCGCTACCTCTCCCAAGTTGTAATAATTTAATAGTTACAAC TATTAGTACAAATAGTTTTAAAAGTCTCTTTGAGTTGCCACCCTGGAGTGTGTATCTCCTAATGGGATAATACTTAA GGCATTTTATAAACTTTAAAGTGCCATGGACATCAGTCATTTGTGCATTTGTGAGCACTGCCTCATTTAAGCCTGCC GTCGCCTTAATGTACTGTGTACATTAGATACGACTTCAGTTCCCTTATCCCAGGAGGGCTGAAACTTACTGGGATTT CTGTTATATTGACCTAACAGATTAGGATGATTTTAAAATTTTACTCATTTTCTATATTGAATTAAAACAAGAAATTA GCTGAGCATTTTCCAAGATGGAAGGAAAAACGGCCATTGTTTTGATTTCTAGTTATGTAACCCATTCCAAAAATTCA AACATGAGATTACACGGAGTGCTGTATTAAACAGATGAGTACCTGCTCACTGGCTTCCTTTCACACAGTCGCCTGGC ACTGCAGCCACACCTGTGCTCACCTGTACCTGCCTACCTGTGGCTGGGCCAGGCCCCAGGAGTCCCGTGGGTCAGGA GCATGGCATGGAGGGTGGGCAGAGCTTTCTGCTCATCCCCGGATCATGGCAGCGTCAGATGGAACTCGGCCTGCCCC GTTTCTGTGCAGCTGTCAGGATGTATGGGGTTTTCCCATGCTCCGTGCCTCCATGCCTGCGGCACTTTGGAAGGACG TTAAGGCAGGACATCTTTAGGAGAATGTCTTTATTGACTTTGTCTTTATTTACCTTGGGCCTGGCGCTGTTATCTCA GCTGAGTGTCCGTGTTTAAAGGCGACACATGGAACGAGCATGGCTCATAATTTTGAGTTTCTTCTTCTGGAGCCTTA CTATTATATGCTCAGAAACCATCTCTGTCTGCGTGGTATCTCTCCTCTGGCAGTGCAGTAGGTCACACATCGTTGAG GTTATGAGCTTTGGAGACTCTTAGACATCAGCTCTCATGTTAACTCTGTGATTTTGGGAAAGTTGACCTATATGTTT TTGTTTTCTTTAGTGTGAAACAGGAATGCCTTCTGCATAGCGAGGCTGTCAGAAAAGCCCTTGACACTTTCTGGTCC CAGAGAGGGCACCGCAGCCACCCAGGCCTGCCCGTGAGAGGTGGCAGCTCTGGGAGGAACACTCCTTAGGCCGGTGT GTGGTACTGTCAGGTGCTTATTTTTGTTTCACAAACTTGGTATTTATTAGGTGTTCCAGGCTTTGTTCGACCCGGGT GGGAGGGAGGATCTTTGTTGGAGGACTCTCACTTTCTAATTGCGGGTTTTTGTTCTGAAGCCATAGGGTGTGTTGCC GTCTCAGCAGGGAGGAGTATTCCCCATTAACGGGCAGGTGGCATCGTGGGAGAAGCACCTGCTGTGCCCGGGGGGGC GGGACTGCGCAGGATCAGGGGGATGCGCGGGAGCCAGTGGTGCTGACACTGCATACCATGTGTCAGGAGCCTTGGAA GTCGGCTTAAATGGTTTAATCTGATTTAATCTTCACAAAAACCTTGAGGGATAGGCATTACGATTAGGCACCACAGT AGAAACTGAGGCATGGAGAGGTGCAACAGTTTCCCTGAGGTCACACAGCTCTTCAGAGGGGGAGACAGGACTTAAAC ACGAGGAGGCTGGCTCCAGGGCCCGTGGGCTTTGAGCGAGGAAGGCCTTCTAGGTCTAGACGTTCCTAGGCTGGGGC TCAGTGATACCCTAAAGCCCATGCACACCTCCTCAGACCTGGCTCCTGAGAGGCAGTCCTCCAGAGTGGCCTCTTTG CTGAGACTGTGAGCCTGGGCTTCTGTCTCTAACCACTAGCTACAAAAGGATGCTGGACAGACATCCTGTACCTAGGG ACACCCCATGATGGCTTGTCCCCCGACCACAGACACCCCATGATTGGGGATACCCCGTGACCATGGATGCCCCATAA CCACAGACACCCCACAACTGGGCAGCTCAAGGCAGCAAGTGCCTTCCTTGTGTGCTTCCGTTTTGTTGAGGTCTTTG TTGCAAACATCGTTTGTTGAGGGCTGCCTTACCCTTAGAAGACATAGTACCATTTCTGGGAACCATGGTTGCTGTTG AGAAATGCATCCTTACACGCCCAGATCCAGCCGCTGTCTGGTCCCACTGACATGGCACAGGAATCAAAGCAGGTGCT TGGGGGATGTCAACAGTTGAATTTGGATTCCTCATAAAGCAATGTTAGCACATGACACTCTGTGAGGAAATAATGAT GCTACTTTGTAAGATAAGAAAGGGGAAACAGCTGCGTTGGTGGATAATTGTTAACGGTGCAGCTGCAGTGTGAGTTG CACACTGGAAGGTGTTTGCCGTGACCTGTCGCGGGCTGAGCTTTTTGGTAGCCTGGCATTGTGGTGGATGCCTTTCA TTCCATCCGCTGTGGCTGAGTTCTGCTTTCCTGCCCCTGGTTCTGCAGTGGCGTGACTGGGCTGCTTACAGGCCCTG TCTTTCCAAGCCACCATTTAATTGGGGAGGGCTACTGACATGGCACCATTGAACTTGAGACCTGCCATTCCTCCGGT GGCAGTCCTTGGAGAACAGGGCCCCAGGTCTTTCTGACCCCACATCATTGGCATGGTCTCGCAGCCAAGTAGGTTAT GTTTTCTGAGGAAATCATCATGTCATTCCCATCTACTGTTAATTTTTAAAAAGCTGTGATTCCTGATGAAACAAACT AGAGATGATGCAGGAACTCTGGATCTAAATGGTCAGTCACTATCTTCTGCAACTTGCTGGAGTGCAGACACGTCTCA GAATAAGATATTCTCAGACTAGTCAGAACATTGTTTCCTTTCTTTAAAAAACAAAACAGAAAACCACTGAGCCCAGA GTTCAAGGCCTCCTAAAGAGGAAGAGGGCTTTGGCTACCTGCAGATAAGAAAGGTTTTGGTGGTAATGCTCAAGTGT GAGTTAGGTGTGGGCGTGAGCATGCCCAGTGGCCGTCCCGTCCTCCTGGAGACAAAACCATTTCCGGGGTATCCCCT GGCTAAGCATTTGAAAGCAGCAATTTATTTTCAACAGTTTCTATCCTTAATATTTAAAAAATAGGAAAGTGTACTCA ATCAGCTTTCTGTTGACTTTCTGCTGCCTGGCCGGCGTCTTGTAGGAGGCGCTCTGCCTGTCTGTGCAGGAGGCTGT CCAGGGTGTGCGAGCCACGTGGGCCAGCCTCTGTGGGCGGACTGTCCCAGGTGGGTGGTGGTGAGGGGGTGAATTCC TCCTTCACATTTGCTTACAAGTGCTCCTTTTTTGAGTGTTGGGAGCACATCTGTGTATGAAGGACATCATTCGGACG CTTTCCAGGGTGGTGGGAAGCAGCCTCCCTTAGCCCGGCTACGTCTCATGAATGTGCGTGGACCCCGCCTGGCCTTC TGCCCGACATGTACTCCACCCCGCCTTCTACCAGGAGCCAAGGCTGTGACCATTGTGATCTGAACAAGCCGTTGAGA GTGTGCTCTGTTGTCAGTGAATTAGTTCAGAGAGTCCCTCCTCGTGTTATAGTTCTCATGACCACTGCGCAGTGTTG CTGTGCTCTGCCTCTGACGGGTGGCTGCCAGGCATTGAAAAAATAACATCGTAAGTCCTTACTGCTAACACTGACTT TTAAAAACATTTAATTGTGTGCCTTTTAAACTTTTGTTCTCCGTACAACGTCTTCCCGATATTTAGATTTTAACTTT TAAAAGACAGTTACAAATTATTTCTAAAGTATAAAGTCTGGTTGTTGTTTTTTTTTTTGATGGAGCTGTGAGGAGGA AAAAAATGAGATATAGAATTTTAGTGTAGGTGATCTTGTGCTTTAGGATAAAACCATTTTTCTGTTACTGCTTTTTG GTATCTAAATCCCCGGCAGTTTCCCCCTTCGGATAAAGCATCATGTAGGTTTTAGCACACCAGCGGGTTCCTGGCTC TTTAGTGAAAAGGTGGCTCTGAGGATTGCCCTGTGCCCGCCATGACCATCCTGGTGGCTTGGCGACTTCATATTGAC TGCAGGCCCTGTGGACAGAAAGAATTATTTCTTTTTTAGAGAAAAGATATTTATGACCGAAAACAGTGCTAAAAACA TGAGAATATTGATCATGGTTTTGAGAGATGCTTGCACTGAATCACTTGATATACCTAAATATCGGCTTTATTCTATC TTCCCTAAAAGACAGATTGCCAACACAAAAGATGAACCATCTTGAGGGAAAAAAATCTTATATCTTTCTGATAGGCT GTCTTGCTTCCTTCCATAACAATGGGGAGAAAAGAAGTCTGTGTGGTTTCCTGAAATAAGCAACAAATATAATAAAT GGTGTATCGGAAGCTTCTGTAAAACTGCCTTGGGTGTACTTTATTTAACTTGTAGTAATAAGTTAAATTAACATCAA ATGGAAATTCAAATAGCTAAACATTTAACCAATCTAACAGTAAACAATTATTCTAAAATCAACATAAAAATAAAGAC CCATTATCATCAGAAGACCTAATTCATTGTTTTTAGAGGTAATCTCTTGGGGTTCTGTGGAAATTTATAAAGGAATG AGTGAGGCTAATTATTTACTCATCAGATTTTTGAAGGCAAATACTTTTCTGTTATTCTTAAATTTTAGACCTATTTT GGATCAAGATGGTGTGTTGCTTTTGTAGTTTAAGGTAGAAGATAGTGAGGCTGACTTCCTTCCTTATCCCGGGTAGA AAGCCTGACTTTTTCTGGATAGATTGCAGCCTAGGGGTTGTGGTTTTGGTCCTGTTCTGCCCTTTTCCCAAATTCTT CTTCATTGCATCTGATACATCCCCCTTGCCTGCAGGGCAGGGCCCTGTCTCTGTGGCACACATAGCAGGACCGTTTA CATTTCCTCTGCTCACAGGTGCCGCCCACACCTTGCTTCCTATTTCATGTCCTCCTCGTCTCTGTAGTTGAGGGAAG AGTATTCCATTCAACAGATGGAAACACCATTGCATCTGACTGCCATGTCTTTGAAACCTGCCAGACCAAGTCTTGAC TTTCGTTTTGTTTTTTTTGGTGGAGCTGGGAGGAGACAGATGGGCCATACAGTCTATGTGTGGGTGCCTTGGTGTGG GACACAAGAAAGTGAGGCAGGACGTGGAGCACCAGGTCATCAGAGCTGGCAGAGGCTGCTCCACCGTGCACCGAAGA CTTGGCATGTCAGTGGAGCCAGGAAGTCTCCGATGCAGAAGCGGAAGTCAGTGTGGCCACAGGAGCGAGGTGGCAAA GTGCTGGCCTTGGAGGAAGAGAGAGGTCAGAGAGTAGACAGAGGGTGAGGCCTGGATCATCATCAGATGAGCAGACA TGAAAGCCGCTCTCAACAGGATTAGAAAACACAGTTTCTAGGGGATTCTTGCAGGGAAACAGACATGGAGGGGCACC ATTTGGTTTGTAAAAGGAAGGTATAGCCCTGAAGTTGGCAAGCTCTTGGCCTGCTGGCCTGATGCAGCAGGCAGCTG AGAATTGCTTGGCCTGTCCATGGGGGCCTGACAGGTATCTCAAATTTTAATTGCCAGCACACACGAATCAGGAGATT TCATATTTCAAAAAGCATGTATGGCTGCTGTTTCTTTTTTGTATAAGGCTTAATTTGTTTTTTTTTTCATGTTATAG CTTTCCTGAGATACATTTTACATACTCTAAAATTTACCCATTTAAATTGTACAGCATGCTGGGTTTTCATGCATTTA TACAGTTGTGTGTCCATCATCACAGTGTAATTTTAGAAGATTTAAATCTCCCTCTTCCCCACTGCCCCTGCCAAAAA AGGGACCCCAGACCTACCAGCAGTCATTTCCCATCCCCCTCCCTCTGGTACCAGCCCACCACTAGTCTGTCTGTGTC TGTGGTTTTCCCTGTTCTGGATGTTTTGCATCAACGGAATCCTGCACCATGTGGCCTTTTGTGCCTGGTTCCTTTCC CTTCGTGTGGTGTTTTCAAGGTTGCTCCACGCTCCAGCATGTGTCAGAACTTAATTCCCTGCGGCCAAACAATATCC CATCATGTGGATGAACCACATTTTGTTGATCTATACATCAGTTAAGTCAGTTTCGCCTTTTGGTGTTGTGAATAAGG CTGCAGTGAACATTTGTGTATATATGTGTTCATTTCTCTTGGGGTATATATACACACACACACACGTAGGCCTAGGA GTAGGATTCCTGGGTCCGATGGCAATTGCATGTTTAGCATTTTGAAAAACTTATGGTTTTCTAATACAGCTGCCCCA TTTTACATTCCTGCCAGCAATGTATGAGGGCTCCAATTTCTTCATATACTCACCAACACTTTCCATGTTGTCTTTTT GATTTTAGCCATCCTAAAGGGCGAGAAAGATATCTTCTTGTGTAAAACGGGCAAATAATCAAGCTGGGAATGGTAAG CAGGGTTTTTATTCACAATAGGGCTGGTTGATGGGATTCACAATTACAGAAGGCCACAGAGGGAGGACACACTTGGT GTACTATTTCTTTTTGTCAAACAATAGCATTATTGAGATGTAAATGACATACAGAGTATACATACAGTAGTTCCCCC TTAGTCATGGGGAATATGTTACAAGAACACCAGCAGATGCTGGAAACCGTAGAAGGCACCAAACCCTTGGTATCCCT GAGGGGTTGGCTCCAGGACTCCTCTCGGATACTAACATCCGCAGGTGCTCAAGCCTCTTATATAAAATGGCATACTA TTCTTATATAGCCCAGAGACACCCTCCCATATTTTTTAAATCATCTCTAGATTACTTATGATACCTAATGCCTAGAT ATCACTTCATTCTTGTGAATTCAACGTAGTCCTTAACATATAGCAAATTCAAGTTTTGCCTTTTTGAACTTTGTAGA ATTTTTTTCCGAATATTTTTGATCTGTGGTTGGTTGAGTCCGTGAGTCCGTGGCTGTGGAGCCCATGCATATGGAGG GCCGGCTGTGCAGTATCTTTTCCTACACATACATCCCCATAATAAAGTTTACTTTGTAAACTAGCCCAGTGAGATTA ACAGCAGCAGTCTATGGAACCATTATAACAACATGCCAGCATCACTATTCTTGCACTTCAGGGCCATTATTAAGTGA AATGTGGGTGACTTGAACACAAGCACTGCAATGACCTTGATAGTCTATCTGATAGATACCAAGATAGATGGCTACTA AGCGACAGAAGGGCGGGGGTGTGTACAGTGGGGATGCACCGGACAGAGGGATGATTCGTGTCTCAGGTGGGATGGGG CGAGATTTCATCCCACTACTCAGAATGGTGTGCAATTTAGAACGTATGGATTATTTATGTCTGGAATTTTCCAGTTA ATATTTTTGGACCACGGTTGATCTCAAGTAATTGAAACCATGGAAAGTAAAACTGTGGATAAAGGGGGCCACTGTAT CTCACATATACAGTTTGATAAAATTTGCCTATGGATGACCCCAATCAAAATAACAAATATTCTCATCACCCCCAAAG TTTCCTCTTGCACCTCTGTGATTCCTCTCCTCACCTCAACCCGCTTTCCTTTCTCATCCCCAGGCAATCACTGATCT GCTGTCACTGTGCATTAGTTTGATTTTCTAGAATTTTGTGATGCACATATATACATATATATTGTATTTTACACAAA ATTTTCTCGTTTTATGAATGGAATTATAACGTATAGTCTCTTCGTGTCTGAGTTCTTCCAGTGTATTGAGTTTGATA TTCATGCATGTTGTTGCATGTGTCAGCGGTTGATTACTCTTTTTGCTGAGTGGTATTCCATTGTGTGGGGATACCAC AGTTTATCATTCGTTTGTTATTGTACATTGAATATTGTTCTTTCTGCTTTCGGCTGTTGCAGACACTGCTATGAATA TTTATATGAAAGACTTGGTAAGGACGTATGCTTCTAATTCTCTTAGGTAAACACCTAGGAGTGGATCCTATGGGCAA TGGGTGTTTAGCATTTTAAAAAGTTGCCAAACTGTTTTCCAGAGTGGCTGCATCCCTCTGCCGTTTTTATGAACAGT GGGTGGGAATGCAATTCTTGCATATAGTCTCTGATTTCATATCATTTTCCTTTTGCCTGGAAGACTTAGCATTTCTT CTGTGGGGTCTGATGGTGATACTGTCTTTTAGTGTTTTTAATGTCTGAAAAAGTATTTTCATATTTAAAAGATATTT TTGCTGTGCAAAGAATTTTGAGTTTTCTCTTTTCCTTTTCTTAAAGATCTTGTTCCACTGTCTCTGACTTGCATTGT TTCGGACCAGAAATCTGCTGTCATCCTTATTTTCCTTCATGTATGTGGAGCATTTTCCCCCCTCCAGGGGTTTTTGA GTTATGTTCTTTATCGCTGGCTTTGACCATCTGGCTCATTTTTTGGGTTGGTGTGCTTGTCTTCATGTGTCTAGTGC TTGGGCTTCATTGAGTTTCTTGGTGCCATCAATTACATCTATATTTGTCTGTTTTAAGTTATCCCACAGTTCTCTAA TATTGTGATTTTTTTGTTTTTTTTGCAGTGTTTTATTTTGGTAGTTTCTATTATGTTTTTGAGTTCACTAATCTTTT CTTCTGCAATGCCTAATATGCTTTGAATGTCATCTAGTATGTTTTTCTTCTCAGGCATGGTGATTTTAATCCATAAA AATTTGTGTCATTTTTATATCTTAATTTTTCTTTTTCTTTTTTTTTGAGATGGAGTCTTGTTCTGTCACCAGGCTGG AGGGCAGTGGTGTGATCTTGGCTCACTGCAACCTCCTCCTCCTGGGTTCAAGTGATTCCCTGTCTCAGCCTCCCAAG TAGCTGAGACTACAGGCACAACCCACCATGCCCAGCTAATTTTTATATTTTTATCATGATCCACCCGCCTCGGCTTC CCAAAGTGCTGGGATTACAGGTGTGAGCCACCGTACCCGACCTATATTTTTCTACTCACCTTTTTGACATGTGAAAT ACAGTTATAACGACTATGTTAATATCCTTGTCTGTTAATTCTAACATCTTTGTCAGTTTTGGGTTGGTTTTGATTTT TTTCTCCTCCTAGCAGTTGTATTTTTATTCTTTTATGCTTTTCTTGTAATTTTTGAATGGATGCTTTATATGCAAAT GTTACCTGGCTGGGTGCTAGGTTATTTATTTAATAAATATCCTTTAGCTTTGTTCTGGGAGGCAGGTAAGTTACGTG GAAATAGTTTGAATCTTTTGGATCATGCTTTGAAGATTCACTAGTTGCAACTACAGTGGTATTTAGTTTCAGGCTAA TTATTCCCACAACCAAGGCAGGATTGTAAGAGGAGAGATCATGGCCGGGGACTCATGGCTAGACAAAAAACAGGACC TCAGTCCTATCAGTGCAAGGAAATGAATTCTGTCAATACATAGTGAGCTTGAAAGAGACTCGCAGCCTCAGCCCTGG GAGTCTCAGTTGCAGCTGACATTTTGGTTTCACCCGGAGAAACCCTTAGCAGAGGACTGAGTCCACCCGTGCCTGGA CTCCTGACCCATGGGGCGGATGAGACAGTAGATGTGTGGTGGTTAAACCACTGAATCTGTGGTAATTTGTGGTGATT TGTTATGCAGCAGTAAGAGGTAATACCGTATGCATCTTCTTGCATCATTTACCAGCTTTTTATTATATGTGGAAGTC TTACGTCTTTCCTCCCTTTTTTATTGCCCTCTGAGTACCTTGTTGTCCCTTTCTGTGTCACATTCTTCCCACTCGCA CGCATGCATCCAGGAATAAGAGCACTGTGGCTCGGTGTGGCCAAGACCCACTGTGTACCTTCCATGAAAGATGTTTG TGAGTAGCATGGGCCCCCGAACCCTGGGTCCAGTCCTGCCTGTACTGCGAGCCAGCACTGTCATCATGGATAGTGCC CTCAGCCCATACTCTCCTCTGTGAAGTGTGTGTGCCAGCACACTGACATCACAGGGTGCCTGCAGCATTTGGAAAAC ACGCAGCATCATGCAAACTGCCTAGGAGTGCTCAACAATTTGGAGCTGGTATCTTTACACTTACCACGTTGAATATT GCATGCAGACTACATATTTGCAGAGCTTAATGAACAGAGAGGCATTTTAGGACAACTAGGAGAAACTCGCGCAGATT CCCCTCAGGGGGAGGGAAGCTGTTTTGTAGGTGTCTCTGGCAGTAGCCTAATGCAGTTGCTTTAGACCCTGGTGGTC TGGATACTGCTTCACTGTAGGCTTTAATTGCATTGCTCCAGGTCTGTGGAAGGCTGGAAGAGGCCGGTGTTTTTGTG TCACTTTTGCTACCCCCTACTCTCTCTAGTTGACAGGCCATTTGGAGCACCAGGGAGTACAATCTGGCTGAAGAAGC GGTATCCAAGGAGGACGTCATGGTGTCTGTTCTCGCAGTCATGGAGCCTAGTGCCAACAGGCCTTAATTCTGTGTTC TGGTGTCTCATGAGTGGCTCTGCAGTGGGACGTCAGAGCAGCCAGGCCTCAAGCCGCCTGGCTGGGGTGGGGACAAA GTGTGTGTTGTCCCTTCATCACGGGAGTGTGGGGCCTGGCACACGCCCTGCCAGGGCCTCACTTGGGCCATGCCTCA GAGCATGGGTGCTACTCACCTCTGACCTCTTGAGTTTTGCAGGCAAACTCAGGTCGAATTTGGATTCTCAGTCGCAT TGTGGCTCACATGACATGAAATAAAATGTTGACATTTATTTGCTTCTTGTGGCCTCTACTAAATATATAAAATAATC AAAACTGAAACTTTCCATTATTCTCAGCAGGAGTGAAACATCTTGACTTTGGAAATTTTTCCTAGTGATTTAACTCA AGTCGGATATCGGCAGCACCACCTGCAATAGTGACCCCCTATGGCCTGCGGCGGGGCTGCTTAACGCTCACATGGGT GGTCACCAAGGAAGGGCTGCTGGACAGGCAGACTCCCAGAGCAGTTGCTACTTACAAATCCAGCTTCCAGCAGGGAG ACCATTAAAGGGCTGCTTCCACCTCCAGGGCGGGCAGTGAAGCCTCCAGCTGGGAGCCGCGTGAAAATCAGAGCCAA TGGGAGCTGGCAGCCCATCCACCAGGAGGGTCCAGAAGACAGAGGCAGAGGACGTGGGATCTGGAGCCCAAGGGAGG GGTCCCAGGTGGGTTGAGGCTGGAGATGGCCCAGGAAGCCCTGGTTTGGGGTGAGGGTGGGCGAGACTGAGAGGCTG CTCAGACAGGATGGGTGGTTGAATGGAGTGCGGATCATGAGATCATCACAGGGCCACAGCATATCAGGCCATGGGTT CCCTGTTAATTCCTCTCCCACCTCGAGGACCGGAGCTTCTGTTAGGCAGGCGGGGGCTCTGCCGCAGGCCCACATTC TCTGCTAATTCCCTTCTCTCCTAAAGGTTTCAGATCTGTCTGTGGTCTTTTGTTTTACTTGGAAACACATGTTTAAT ATATCTTATAAAATACGTTTAATATAAAAATATAGTTCACTTTATAGTTCAGTTGCTTTGAGCACATTTAGTAAACC AAGATGTGCTTATATGAACTTTTCCCTGAAAAGTACCTGGATCCCAGAGTTCTACATGGATTGAATACTTATGTTTC GATTTTAATTCCTTAGATGGCTAACTCAGCATTATTTAAATTTTAATAAAGAGACTGGATACAATGTGAGCGAATTT CTCTCTATATTTTGTGAATCTCTATCAGAGCAAAATAAAACTGTTTCTGTTTTGCAATGAATAGTTTCTAGCCATGG AATTGCTGGTCGAGGCAAGAAAGTATGAATCTTATTTTTGTGGATCTAGCCAAGAAAAAGAACGTAAACTTTGAAAA GTATGTCATTTGCCATTCACAGTCTAATGAGAAACTCAGAGAGGCTAGAAACTCTCAATAAAAATTCTGATATCAGC ATTTAAGATCCAGCAAGATAGGGTATACGGGCATTTAAGGGATTCACAATCCACAGTTTACTGGCACTTTAAATGCT GTGAATCTTGTACTGGCAATCACAATCTTGATTTTATTACCGAGGGGGACCCCTCGTGAGGAATATGTCGGTGCAGT GTGCAGGTTAAACTGTGAGCTGCTCCAGCCTCTCTCCCGCCGGCTGACGGAAAGGTGGTCAAATTACCTTCGCAAAT CTGAAGGGTGCTTGGCTCCAGCAAATTGGCCATTTAGTCAAAAAGTGTCTGAAAATGACGTAGTCGTTGGAAGTGTT TGGGAATAAGCGATGAGTTGGCCAAACGCAGTGTGTTTTCTGTAGGCAAGCACCTGTGGCAGCTGGCAGGGATGGGC AGGCCTGCAGAGATGGCTTCCTGGGGGCGCAGCGTGGTCAGGCTTGAGGGTGGTGACAGTGCCCCCAGGAGAAGGCG AGACTGGCCAGAACTGGGACGGGCATGATGAGTGGCCGTGTTTCAAATATTGTCCATTTTTAACTTTTGAAAATGCC AAAAGCAAGTATGCTGCCTTGTGGAATCTGTGCAAGCAGTTCTTTCCAGGCTGACCTTGAATAGCCTGACAGCAGAG CGAGGAGTGAAATGAGCACGCCTCAGGCTGTGGTCCACTCGTGCGGGAAGACATGGACCTGCTACTGCTCCCTGCCC TCTGTCTCCCCAGGAAAGGAGCATTCTGGCTCAGAGATTCTCAAACTGGGTCTTCCAAGACCGCACAGGTGCTGTGT CAGGTCAGAGATGCCTCTGTAACAACGCCAAGACCTCGCGTGTCCAAGAGTGATGCTGGGGGAGGCTGCTCTAGCTG GACCTAGATCCAGGCCAGGCCCCAGACCTCTGCAGCTGTCCACCTACTCCTCACCACCACGCGTTTGCAGGTGGTGG ACGGTGTTTTATTTAAGAATGTCCTTGTTGGCCGGACGCGGTGGCTCACGCCTGTAATCCCAGCACTTTGGGAGGCC GAGGTGGGTGGATCACAAGGTCAGGAGTTCGAGACCAGCCTGGCCAACATAGTGAAACCCTATCTCTTCTAAAAATA CAAAAAATTAGCCAGGCATGGTGGTAGGCACCTGTAATCTCAGCTACTTGGGAGGCTGAGGCAGGAGAATGGCTTGA ACCCGGGAGGCAGAGATCACAGTGAGCCGAGATGGCGCCACTGCACTCCAGCCTGGGTGACAGAGCGAGACTCCGTC TCAAAAAATAAATAAATAAATAAAAAAGAATGTCCTTGTTGAAGCAATAAACGCTAATTGTACTACAAACTGACCCT GGAGCACCTCTCTTTTTAGTAGACACGGGGATGAAGTGAAGTGTGCGTAAAGCACTTCTGCCATGGACCAAAGCAGG GATGTTTTCCGGAGGAAGGGTTTGCGTGTGACTCAGGGTGACGGGAGTGGCCACTTTTTTCCTGGAATACCATTTTC ACCATTGAAAGTATCACTGACCAGCCAACCATATTTACCCACACTTGGGTTTTTGGCAGAAATTTTCAAGGAAAATA GCTGACAGTATTTGTTGCTAGTACTGAATGATGAAATCTGAGTTTCAAGTGAAAATCATAATTTTGAGAAACTTGCA CCTACTGCCGTGAGCTTAACAGCATTTCAATGTTTAAACAACGTTTTGCTGATACCAGTGTGGCATTAACTAGTGTG AATTTCGGTAGTGTGCTGTGGCTGAAATGCCTAACGTAGATTTGCATACCTCAGTGAACCAGTATTTTCTAAATGAT GGATAATGTTATAAAATTATGCCTGGGTAAAAATCCAAAATGCAGGCTAGGTGAATGGATTTTAATGGAACCGTGCA GACAGTTGATTCGGAAGGCTTCAGGGTCCACATGGAAACTGTGGTTTGGGAGCTGCCCCTTGTTGAGAGTTCTGTTC AGGTATCAAAGGAGAGTATCCACAGTGGCCCAAGGTAGATTTTCTTCCTGCACGTCAGCCAGAGCCCTGTATGGAAA CAGACTGAAGGCAGAAGCAGCTGCGAGAGTCCACCTGCCTTCTGTTAAGCCAGCTGTGGAAGACAGTCTCAAAAATG TAAACCAGTGCAACTTTAATTAATCCTTTTTTTTTTTGGAAAAATGGTTATTTTTCATGAAAATGTTATATGTGATA ACATGAAATCAGTTCATAACTATTTTGAAATGAAATATTTTTAAATGTTCTCAGCTTTCATTTCAAACACAGTATCA ATAGAAATAACTTGCATGAGGAAAAAAATAGCTCTTTAGAGTCCTCATGATGCTTAAGGGTGAGACTGAAAAGTCAG ACACCCACCGCCCCAGACAAACACCATGGTGGCCTCGGGGATGCTGCGAGGTCAGAGAACCTGCACTCTGCTTCCTT CTTGTGTAGGGTGAGGGAGAAGCCCCAAGCCGTTTCTTCTCAGGGCTCAGGTGTCTCTCATCAGTGTAGGGAGGGTC TTCGGGCATCCTATGCCCTGCTCATTCCCTCGATGTTATCCGGACAGGAGATTTCAGCTCCTGCGCAGTGGGCGCTG TTGGGATTCCCAGGCCTCCAAGTCATGACGGTGGGTGAAATGGAGCTGGTATTTGAGAAGGCTCGGAAGCCTCCCCT TAGAAAGGTGGGTCCTGATCAGCAGTTTGGGATGCCCCTGTGTTTCCTGCATTTGGGATGGGCGGTCCTCCCTCCTG CCTCCCCAGCTCTCTTGAGCACAGTGTGGCCGGCATCTGGTCAGGACTTCAGAGTGGCCAGCCCCGTGTTGCCGACG AATCTGAGAGGGAGCCCCTGGCTTCTTATTTTTCTTTGAACTACGTCAACGGAATTGCTTCGGATCCTTAGCCATTT TAAAAAACACAGTCATATCACGTCCCCTTCTGACATTCTCTTAAGCTTTAGCAAATAACAGAAATTTCAGTCTCACT GTGCCTTGTTCAGAGGTGACAACAACAATGGGTGGCTGAGGCGTAAGTCTCAATCATCCAGGCTCCTTAAAGCAGCT TTCGGGCATGTGTGGGGAAAGTACGCCACAGACACCCAGGGCTGTGCTTCCAAAGAGGTTTTCAAGAGGTTGAGTAT TGACACCTTTCCTTACAAGGTAGTGGGGAGGCCTGGAGGCCAGGCAGTGAGGCGAACAGTGCTCAGTGAATCCACGC TCCAGGTGAGGGGAGGCAAATGTCGGCAGAGAAGGTGGGAGCAGAGGAGGAGTTCCTTATGCAGAGGTCTCTGGGCA GGCAGAGGAAATGAGCCTCGACTTTGCTCTGCCCTGGGAAGGGAAGCTTGTGATCCACATTATATCAGTGTGGGGTT TTATGAGCTGGACTGAGATGGTTGATCTAAAGTCACAGTTGGCAAGTCCCTGTTTATGCGAGGCCGGCAAAGAATTC ACCTCTGAGTGATCTGTGGGGGCAGCCCTGTGTTGGTGCCTGAGGCCTCTGCCTTTCTGTGGAGGTCTGGCTGATGC ATGAGGCTAGAAACAGCCATTCATTTGGAAGAGGGCGTTGCAATGACTCAGCCTCTGGGCTTAAGCTTTCCTTTTGC ATAAGAAGTCTGAGGGGTCCTCAGATGTTTTATCTTCCTTTACAAAGTTGTGTTGTCAACACCAAGGAAACAGTCTT GATTCTGAACAAGGAGGTAGCCTGGGTACTGAAGGTGAAAAGAGCTCAGATCAGAAGTTTCTTGGAAAGAGATTTAA AGATTTTTAAAAAATCAGATTACTAAGGCATATTAAAATATGTTCAGTCTTACACTGTGTTCTTTGAAACCTTGAAT TACTCTAGCAAAAGGCTCAGAGGGTACTGCTATGAGAAGCTGGATCTTGGACATCTCTTTCAAATGCCTCGGTACCT TTCAAAGGGCTTTGGATGGAATGAAGGGACACCATTGGGTCTCACAGCTTTCTCCTGCCTAACATACCACAGCAGTG TATGAAATTGTTCCCATCAGCTCCAGCGCGTGAAAGACAATACGAACAGTAACACCCCCAAAGAAACAGTGGGGCTG GAGACAGCAGAATCCTTGGAGGGTTCTCTGGCTGAAGAAATCTCTCTCCTACCACAGATACTGTCCCCAGAGAAGGT GCACCTCTACCACCAATGCCCTCCATCCTCAGTTGACAAGGATTCAAAAAGTTTGTTGGTGACGCTGACATGTAATA TTCCCCTGCGAGTTACACACATCCAGGTGACCTTCGGACCAGCATTGCCGGCACACCCGTCACGCCATCAACCTGCC AGGCTGGAGAAGACCGCTCAGAGCTTTTCCTTGACTGGGGAGAGGGGAGCCGGAGCAGCGCCCCACGTCTCACGGTT CACCCCTGCCGTCTCTCGGGGCCTTCCTTCAGGCTTTCCCTGTATCCTCAGATCTTCGCCAGGGTCCTGTGATCCAG TGTTAGTTCATTCCATGTGTCATATATGAATGATGTCATTTGAATCATTTCAAGTCCTTTTGTAGGAAAACTAAGGC AGAAAGGCCAGCACCACAAAGAGGCATCCAGAATTCTCCTCGGCACAGGAGGGTGCAGCCATGGTGCCTTTGAGGAG ACGGACACGCTTAGTTCCCCCTGCCATCAGTGGGCATCTCGATGTTAACCAGATCTTAGAAATTGCTCTGCTCAGGT TGGACTCATAAGTGTACTCTTAGATGCATTCTTCCATGTTTTTGGAATTTCCTAGAGAAAACGTCTTCTTTGTTCAT GCCCGTGACACACGTTCCTCCCTGTGGTGACCTGGCGTCCATCCTGAGCTTTCGTGGTGTCCACCCATGAAAACAAA GGTGTCTGATCCCCCGTGCCACAGACATACTCTTAACCAGAATCTCCCAACCTGGGGTCCGTGGTGGTCTTCGGGGG GGTCTGACACTACCCAGGGTCAGATGAAAAGTGTTGACTGGATGCGTTTATGTGGGCGCTGCGATACTCTTCTTGGG GCGGGGCCCAGAGGGTTCCTGAAATTCTCAAAAGTGTCTGTGTCCCTCCTTGTGAGTAGGAGTCACTGTTTCAGATG CTCAGCTTTCCAGGTGATTTTTCAAATCCCTGGCACAGGCTTTCAAGGTGACTTAAGATTGTCTAATCCTCCTGCAG GTAGAAATGAGAGTCATCTGGCTGATGGTCACGAGCCGTCGGGGAGTTCCTCCTGTCTTGCACTCAAGCAGAGGAGT AAGGAGACCCCTTTAGAATGTACTTCTAATTGTTGAAAAGCACCTAACAAGTTTTCTATGGGGGAGATTATACAGGG ACTGGGTAGGGACAATCACGTAAGACATACCTGTTTATAGAAAGAGCTGGATATAGAATTATAGGTGGGTTCAGCTG CACAAAGAAGAGTCAGACTACGGAGGCTTGAAGGAGCTGTGAATACACTGGGCATCTGACATCATTGCTTACTTCCC CCTGCCCCGCCCTGCCCCTCCCTGCCCCAGGGTATCGGTAGCATGTGTCCTAATTAAATTTTGACATTATGTGAGTT TTAGCATGGTATTTTCTGCATTGGGCTATCTGTATAATGTCATGGAGTGGTGACATAAATAATGCTAGACTTTTCCA ATTTATCTACCATCCCAGCCCCCTCCCCAACTCAGTCATATGTGGAAAGTCAGTTTCTTTTTCCATTTATTATAGTT GTTAACTAAAGATTAAAATTTTACATTTCATTGCACTGTTCATAAATTGTTTATAAATACAGTGAAATCTAAGCAAA CCAAGATGATCTTGGAAAGAAGTGTTCTGTATGGAATTTATCTGTTCAGACCCAACATGCACACCCTTCCTTCGGGA TGTATCACCAAGATGAAGGCGGGCAGATCTCATGGGCATGAGCTTGTGTGCACCGAGGAAGTTCCTGCAAGCAGTCC TGCTGCACCTGGGCGGGGGACACCCTGTGGAGAACCACGGTGCTTGAGTGGAAGGAGTCTGTGTCTGTTTTTCAGAA TGCCTTCTTGCCTTCACAACTAATATGCAACAAAAATAATAGGATTGCACCTGCTTTTGATGCCTCAAGATATGCAG AAATACATTTCCCTCACTTGAGCATGTCTCTGGATATACTAGGACAGCTAGGTCGTGCTTGCGTTTCAGGCCACGGC CCTGCTCAGGAGTCAGGTTCAGCTTCTTGCATTGAGTCTTATCCGCCACAGCCCAACTTTAACAGTGGATGATAGCC AGGAAAGAGAGGGAAGCATTTTTGGAGCTGGTTGTGGTTTTAATGAGGCATATACAGACACTGCTTTAGTGGATGTA GGAGTGAGGGGGGTGGGGTGTAGTGGTTGAGGAAGGGGCTTAGTGGGCTGGGGTGAAGGTCTCTGCTGGAGATCTAG GAGTTTTCCCTAAATCCTCATCTGATCTTCCTCTCCTGTAGATTGTTAGGATAAATGAGCAAGAAGATTCTGCGCAA CAAACTGATACCTTGCTTTCTTTTGTGAGCAGCAGAGCTGGGGCGTGGAGAGGGTACGTCCAGTGTTGGATGTCAGG AAGTTCACGTCCCAGGGTATCTAGTACATCTGAGCTCTAAGCCTGCAAAGGAACCTTCTTTACAAATTGCACCATTG TTATGGTTGGCATTCTGGTTAAAAGTTCATCTGGATCTCAACCTCGCGTGAAATTGCAGAGCTACGACTGGAAAACT CGGCATCAGTTGAGAACCTTTGGGGACTTCTCAGACATTACAGTTAGAATTGAGCAAAACATGCTGAGTTCCATTCA CTCACATTTTGGTGAGTGGGTTAAAATAACAGTTTAGTTTCGTCATTTTTATTACCTACTTTTCAGACAGATCCAGA AAAATAAAAAAACAAAAAAAAAGACAAGCATCATTACCATGTTTAGGAAATCTTTGAGAAGAAATATGTATGAAATT TGGCATACTGGCTAGCCATTCCCTTACCCCCTTATTTTTGATTATCCAAAAGCTCAAGGTGACTTTGCTCTTCCCCT CTGTGACTGGGGTTAGCAAACAACAGCCATGGGCTAGATCTTGTCCACCAGCTGTTTTTGCATGATCAGGACGCTGA GTGTGGTTTTTTCATTTTTTAAAGGGTTGTAGGAAATAAAGAGACTATTGAGTAGAGACCTCACAGGCCTTGGAAAG TGTGACATACTTCCTATCTCCTTCTCTACAGAAGTGGCTGCTGACTCCTGCTCTTTGGGGTCGAAACACAAAGACTG AATAATGATGTGGTATTTTTGTATTTAAAAAATGGCATAGGGCATTTTTAGGGAGGTTGTGGCATTGACGTATTCTG TGTTGCTTCAGAGAGATGTCGTTCGGTGGGTAGAGGCTGCGCAGTGGCCCACTGTGATTGGCATGGTGGTGGCTCTC GGCCCCTGGGCCTGCTCTGCAGTCAGGTCCACCATGTGGAAGTGCTCTGAGACTCGGTGCACGTATTCAATGTTTAG TGTGTGCCAGCCACGTTCTAGGCACCGTGGATGCGGCCAGCAAGGAAGATGAGGACCCTGTTCTCATGCCCAGCCTC AGGGTGGGTGCATGGAGCATCCCTGGTCGAGTGTGTGCTCAGGATTCCTTTAGTATGTCATGAATCTGTCGTGTTTC TCAGAGCAGACTTAGGAGCCAGGCTCCCTGAGATTGAACCCTGCGTGGGCCACTTCTTTGCTGTGGAACCGTGGGCA AGTCTCTTGTTCTTTCTGGCGGCTTCCATCTGTATAAGGGGATGATAGTGCCAATCTCTTAGCGTAGGTGACAGATT CCGAAGTAAAGGGCTTGTCAAGTAGGCCTGGGGTGGGCCTGGCACATCCTGGGGGTGCTGCCTGAGGGTCAGATGTC ATCTTGGTGGCCCACACTCCCCTGCAGTGGTGCTGCTGGTTCAGGAAGTTAAAGTGGAAAACTGTGCTCCGGAATAA ACACAATTGGGAGAACCCAAAACCACACGCAAGAAAGCAAAATGTGCTGCCCGATTACTAAGCATGGAGTTAAACTC TATTTGGTGTTTTTGTTCTGAATTGTTAGGGAGTAAGAATATTTATGGTTGGAATCCTTGGCCAATCCTAAATTTTC TTCTAACTAGTAGATAATTAAGTGTCTGCTTCTAGAGGATGTAGATGTGCTTGTCTTGTATATTACAAAGTAGAGAC GACTTGCCCACACCCCCTCCTGGGCTCACACTCCATGCGGATGGCGTCAGTAGCTTCTGAGCCTCTTCAGGTGCTGA ACACATGCCCTGAGCTGCTTGTTTTGACAGGGACATCATTTTTAAAGAGTCCTTTATTACCCCAGTTTACTCACCTC TTGTTGTCTCCAGCCCTTCCCTTTTTTATGTTCTTCTCTCCCTTTTCCCTTAGCAATTGCTAATACCCTAGTTGGTA CTGATAAAAGAAAAACTTAAGCTGAATTGAATTTAAAGGAGTTTAATTGAGCAACGAACAATTCGCAAATCGAGCAG CCTCCCAAAGCCAGAGTAGGCTCAGATGGACAGAAAAAGGAAAGTGACAGAGACGAGGCACAGAAACAGCCAGATTG GTTACAGCGTTTGCCTTATTTGAACATGGTTTGAACAGTCAGCTATATTGGATTGGCCAAAACTCAGTGATTGGCAC AAGAGTAGGCTACGGTCTGTTTATACCTCCACTTGTTATAGTTCATGAGGTACAGAAAAACCTTGAGGCCACATTCA AAACATGGAAGGAGGCAGCTTTAGGCTAAGCTTGATTCAACAGGTTGCTCTCGTCACTAGGAAGTAGTCATGTACAA ATGCAAATGTGCTTGAAGTTTCAACCTAGTTTTTAGAATGTGGGATCCTGATGGGTATTCCTCTCATCTTGCTGCTT CTTGTCTTGCGCATTTATCATCCCTTCGAGGCAGTGGTTTCAGATCTTAATCCTTTTTCCTAGTCACCTAATATTCT AAGATGTGGCTATACAAAGCATTTCCAACCATTGTCCTTCCCCCCTCAAAAATTATGGTAATAAGCGTCCTTCCCAC CCCACAGATAATACTGCAGTCAGTGCAGACATATGCTTGTACATGCTTTCACACACGATTCACTGAGGCCGGCAAGT CTGTGGGAGGGAGTTCTATAACTGAATGTTTACACAGGGTGTGCATATCTTTCTCTTTGAATAGGTTGTTTCTATAC TGTTTGTCATGAAAACTCAGACCATTCCCATTTTACCATCCTTTCTTCACCAGGGAGATAGAGTGACAGAGCAACTT AATTGTTGCCCATCTGATGCGGATCAAGTGTCATATTAACTGTGCATATCCCTGCAACGTTTGCACACTAATGTACA TTGGTCTTTCTAGATGCCTGTTGTTACCTATAGGTAAGTAGGTAGGTAGGTAGGTAGGTAGGTAGGTAGATGTCTGC TAGCAATTTGACGGGTCATTTCTTTTTGAAGGATAATGAACAAATTTGAAACCTGTGGCACAGCCTGGGACCTAAGT CTGTGTGGTGCAGGGAGTCTAACTTCTGTCTAAGTCAGTAGTATCTCCAGGGATACAGCCTTACATCCACTGCTGGC TGCGGCTTCTCTGAGCGACCCATCTCTGCGTCCGCCCATCCATGGCTGCCATCTCGCACAAAATTATTTTCCTGGGA TGGTCCCTTGAGTTAGTTCGTGGTTAGGGGTAGCTCTCACCTTAGTCCCTACAAACCCACCTGAATTTCCTTTGTCT CCGAGCCACATGGGAAGGAGACTGTGGAAAATAAACCAAGAGATTCTCTAGTAGGTAGAGCTAGAGGCAACAGGTTA AAATCACATCTTTATAGGAGAATGTTTGGGGAATGCATTCAGCAGTCACCCATTCTCATTAATAGACCCAGAAAACA ATTAAAAAATACACATTGTGATTTGGACTGGGCAGACATGCCGACTGTGGAGCCATTTCTTAAGAGAGGGAGGTTGG CAGGTAGCTGGTGAGCCTATTTCTTTGCTCATTGAGCTAGCAAGAGCAACACCCATTACTTTCATTGGATTACTTGT TTGAAAATTAAATTAATCAAGTGAGGTAAACATCCGTATTGCCTCTTAGCACCCCCATAGGCTGCCCGCAAAATGGC CTGCGTGGGTATCCTAAGAGCAGGAATTGGCTATGACCTGCCGAGGTGGCCAACTCAGATGTGGCCAGTTTTCCATA CAAGTATGAAGTTCCTGAATTAATGGGAAACCTGGCTGCATTGTCTGATGGCTTACTGGCAAGCAGTAATTAGAAAA CATTTTTCATAGTGGAACCTCAGGTTTAGCTATCCAGAGAAATTGGATATTTCAAAATACTCCTAGTTGGTTAGTGA AACATTAGTATTAATGACAAATTAATATTAATATCAATATCAGAGTTGAGTATGTAGCCTTTGGGAAGAGAAGGGAA AGATTTTAAGGACCAGATTTAAGTAGCCTCCATATGCCTTTCCTTCTTGGTGGCATTTTTCATCTGAAAGCATTTTT TTTTTTTTTAGCTCAAAAACTTAGATGTGAAAAATTCAACGCATTGGGAAGAGGCGTCAACATTTGAACCGCAAATG ATTAAACCTTTATGACTAAAGTAATAGGTTTGGCTTCAGTCTTCCCAGTGGAGTAGCAAGCAGAGGTCCTTTTCAGT TTGCAAACAGTAGTATACAAACTTGGTCCATATCAAATACTGTTTCTCTTCCAGGGCTTGTGTGTGCACTGGGAACA GCAACAAAAGTTGAACAGCAACTGGGTGGGGTTTCTTTATGTCTTCTGTTTAATCAGAGTTTCCTGATGGTTTGGCC ACTTCATTCTCCTTTGGCTTAAGGGGTTCTAAGCCATGGGGCAGACCTTGGGTGGCTCACTGCTCTTGGAAGGAGGG TGTCCAGCCTTCCTGGCAGTGTGGAGATGAGTAAGGGAAACACTGCAGGGGCTTCTGGCTTCCTGGATCCTGCCCCT CACCTGGGGACTTTGTCTAAGTCCTCATCCTAGCACCACCTCGAGATCGATTCAGGCCTCCCCAGCAGTGGCAGAAA TCCAACCATGGGAGCTAGTGGGTTCCATTACTGTGGAGTAGGGAAGAAGACATATGTCCCTTCTAAGTCACTTTAAT TTGGTCCAAAATAAATAACCAAAGTCCTTGGGGAGACCGTCAGGGAAGTATCTTCCCACATCACACTTGGTCTTTGC AAGAAGTGCTGGTGGGCGTGCAGGGCTTCTTGACACTTCGCAGCAACGTAAGGATGTGGTAGTAGAGTGCTTGGTGT TCCAGTGGCCACTGCAATACAAATAGAGTGAGTTTTGTCGCCGTGGTACATTACAGCCATGGAAATTCCCCGTAGTT CATTGGTCCGTGACCAACAAAAATCCCCCTAGACTAACAATTAATCAGGCTCTGCTGAGCCTTTGACTGGCGACAGT TGCAGATGTCACATCATGCGAAAAGTTAAATCTCAGCTTTCCCCAAGAAGGAAAAAAAGTTTATTTTTACAGTTTAT GGAACTAGTTTTCCTTTTGATCAAATGACCTGGTCTTTAGGGGAGAAATATTTTCATTTTTATGTTAGCAAGTGTGA TACAGAATCATATACATTTAAAATCTTCATTTTTATTGCTTTTTTTTTAAAGTACAAAATTTTAATAGTAATAGTGA TTTCAAGAAAATGTTGTGAGTGTCACTTTACTAGTCAGGCATGTTGAAAAGGAACGCGCTTCCTGAAATCTCCAAGC GAGGTTGGCATTTGAGACCTCTGGGTACGTGAGCTTAAAATTAATATAAGTACCTGAGCCTAGAGCCCAGGAGGTTT AGAAAGACTCAGAGATGCGCTTAAAGATAATTGGTGTGCTCCTCTTGTGAGAGCGGATTCACCAAAATGAAGTTAAA ATTTGTTTGGGAAGTTGCTTGTACTGTGCCTTGTGAACTTACAAATCTGTGTCACAATCCAAAGGGTTGAGAGCCAC ACAAAGCAAAACAGATGGGCCGTGTGTCGTAGGATTTCCTGCTGTCCTTTCGGCTTCAGGTAACTAGGACTTGTGAA GCACAAGATACATGGGAGGCAAATCTGAAAAGGAATGGTGAGTGCGCCCCACGTGCCTGGGTAATACCTGAGTGCTC CCGGCATTACCTGGGGGCTGCCACCCTCCCTCCCCCTCCTTCCTTTCCAGCTCAACATTGAGGTGAACTTTTTACCA TGATCGATGTAGCTTTCTGGTGTCTACACAGATTTCTGGATGGCCTCAGAAATGTCACATACTGCTTCGGGAATGTC TGCAAGTATATTTCATACATTTTACCTGGCAATTAAAAAAAAGTCATCTTTAAGATATTAGCATGTGGATACCGAGG TTCCACACAAAGCAAACAAAGAGGTCATTCTTAGCAGCTAATTTTAAATCTTCAGGCAACTATACGAACCTATGTCA CAGTCTCTCCCAAAATTTATTATAATGCCCTTGTTATAAATTTAGAATATTTTTGCAAACTCATATAAACAAGTAAT TGATGCGACTAAAATTCTTGTCACATATTTATTGCACTATCAGCTAAAATTTTTAAGTGACAAGAACCAAAATAATC GTTAATTTTTATAGGGCAAGGATCTGGCTTTTTGTCTGACACAAAGCTGTATAAAAAACTGCTTCTGATTTTATGCA CATTGTTTTTAGTTAATGATTTTCAGCTGTTTGTTGTTTCTCTGATAGCATGAAAATGTACCATATGCTCAGCCCAA AGACAGATGATTAGCAAGTGAGATTCCGGGCAGCTGTCTGACTGAGCGCGTTGGGTTGATAGCATTCTGCTATCATA ATTAGCTTAAGCTTTGGGTTAATTAACTTTCTACCTGGATATGCATAATAATAGCAAACTGCAAAAAGCAAGAAGCT TTCAGTACATCAACAACATTGCTGTCTTTTTTATTTGGTATTTCAAGAGCATATTAGAATTTCAAGAGTGATTAATC CTGGGAATATTGTCATCTACTGTAGATTTTATTTGCAAGCCAGATAATACTGAAACAATTGGAATCTATTATAGAGC AGAAATAGCATTTTAAAATTGTAATTAAAGTTTCTAAAAGCAATTGCTAATTTCAAATGCCTTTGTTGGCAAGAATA TTAGGCACACTGCTTCTTGTATGGGCAGCTTGTCGGGCACAAAAAATACCAGCACTTACACTGTGAAGAATAAAACT TTCTTTTATTTAAGGGCTGTATTGAAAATAATCAGTTATTTTTATGTAATTTGAAAGTAAGATTACTCTGTAGTTTG CAGAGGTGCTGTTCAGACTCCTAGATTGAAGATATTAACCCTGTGCCCTCTTGCAGGTTCTCACCTTTGTTACCTCC TGAGCAGTGGAGGGCTGGAGGCCTGGGTAGGAGTGCCTGGTGCCCTCTTAGAAGATTTGATGTGTGCGTGTGTCTGC GGGTGCCAGAGGTCAGATTTTTCAAAAGTCAGTGCTATACATTTGTAGCTTGATATTTGTTTTCTACCCCAAGCAGT TTCTAAATCAATTTACTGATGGTATGAAATGGGGAGAGGGAGGGCTTTGTCTCCATCCTCCTCCTTGTTTGCATGAT TTACCAAAGCACTCTCTACGACGCTCATCATCTGAAGGGGCTGCCGGGGCCTTTGGAGAGCACAAGCTAGTGAAGTG GATGGTGGATTTGGAGTAAAAATTGTAGTTTACATTTCCCAAAGCACCAGAGAGCCTAGCACTTAATTCTGAAATAA GTCATGCCTGTTGTTACACTCAGAAAAATATTTTATCTTCTGAAAAAAAAATTCTTTACATATGCAGTGGATGGTGA AGATTTGGTGGCGGTGAATTTGCCCTGGCTCCAGAGGACTTTACTTACAGAATCAGCAGTCGTGAAAACGTGCTTTC TCAGATGTGTGACTTGTGGTCAGTCTGTGGGCTCAGGAGCACCCATTTGGTCAATCCATGCAAGTTTTATGGGGCTG TCAGAAGCCATTTTTCAAAATTGGCTTCATCTGAGGACACGTGGAGGAGCCACTCTCTTGGTGTGGGTAGTGAAAGG AGTGGCTCCCTGTGGCTCTGTCTTGGGAGTTGGGGGCTGCAGTGGTTCACTGGGAGGAGGGGGTGCCCCTGCAGGCC CCACCTGCACCCCACCTCGCCGCCCCACCCCCTCCCCCCGGCTTTCAGGCTGTGACCTCAAGGCCTTTTATCATTTT CACAATGATGTCTCCAACAGACCTTTACCCCAGTCTCAAAAAAGATACTGCCAGAGCCTGCCTTTGCTTTTTGTTTA GTCTGCTTCGGGCTGCGTCAATAAAGATAGCAGAGGGGTCAAGAGAAAAGAAGTGAACGGAATCACAGTTTTGGTGT TGAACCCTTAAATGGGGGCCCCACTGTCTCTGCTGTGTAGCAGGCACAGGATTATCTGTCTGCCTTCTGAGATTTCT TTTAATGTTTTACCAAATTAAACATGAACAATTTCAGGATGCAAAAACTTGACTGCATCTTGTTGTTTCTTAAATAA GAGCACTTTAAGAAGGCAATATATGGAGATTCTTTTGTGGGAAAGATGTTTCAAATGGGTTATTACTGGCCATTGAA AGAAAGAAAAAGAGGAAATGCATCGAGGCCTCATTTTTTGGCCTGCACATTTGATTATTTTATTTTAAATGCCATTT CAAACATAGTGTAAACTAATGATATTATTTTTTAAAAAACTTACTCTACTATTTGCGGTCCTTGATCTTTAATTTCT TTTAATGTCAAAATTATTTATTTGGCCAGTTCCATTCCAGATGTGACTGTTGAGGGCACATTAATTTCTATTCTGTT TTCATGGGCCCCTGTCCCCATCTGTACATCATTTCTGACTTCCGTGTGCTCCCTAGGGAGTTCACTTATTCATATTG TAGGTTTCATTTGCTGTGTAAACAGAATAAACAGACAATGCAAAAATCCCCACAATACCAGAAAATAATTCTTTTCA ACAACATAGTGGATCATATTTAAAAAAAAACATGGAAAATGCAATACATGTGAAGCCCATTTTTTCCACTTTATTGT CCTCCCGAGGAAAGGCTTGTGTTACCAGTGCATTTCCCTTGAATTTGATAAGTTCATTTTTATTAAAAATCCATCCC AAACAGCATTTTACGATAATTCTCAAACACGTGTTGTGGACATTTCTTTGTTTTGCGTATTAGCTTGGTCTTGCGGG TTAATTCGGAATGTGTTACTCAACTGGTATGTGCTTGCGTGGCCAAAAACACCCGTTGTGCTGTGTGTGTGTGAAAA GCAACATTCAAGAACGGTGATTTTTTTTTTTTGACATTTACAAATAAAGAGGGACATGAGTTATTTATGCTCCAATG TTGCTGCAAAGTCCTTATCTCCATTTGGCACTAAACAAGGATTTGTTGTGGGATTTGTGAGGGCTGCGCAGTGCCTC TGTGCTAAGCACCCAAAACTCTAGGGGACTGGCACTAATTTCCTCTCTCTCGTAAAACAACGTAGTATTCATAACAA CTCAAGAGGCTTGACATAAGTAATGATGGCATGTGCAGGCGAAGATTACCACCCAACAGTGTAATCAAGGTGCTAGG TACTAATGCAAGTTAAATTGAGACAATAATGCCGAAACTTGCATTGTGGGATTAGGAGGGCACTTGAAATGCTGACG TGAGCACATCCTGCCTGCCTGCCTGTCTGGCGCTCCGTTCCTGTTTATACTTAATGACCTCAAAATTTGTATGTCGT TAACACCGTGTACTGAAGTGGGAGAGAACACGGTTTGGGAGGGAGAAGACTCCCTGTCTTCGTTGACTTTTGTCTCT TTTGGTAATTGGTGGCTTTTCCTGCCGTATTAGGGAGCAAAGCGTGCAGCGGGGAGGCCCCTGAGGTCTCTGTAAAC ATGCTGTGGGCACCGAGGGCCTCCGCACCCGGCGCTAGGCAGTAATTTACATACTTTGAATGAAAAGTCATTTGTGC CTGCAAAATTGTTATTGCTGGAAGAAATTTAAATACTAGTCATATGGAAATTTGAAATAAAGTCATATTAAATTTTG ATGCTCAGATCAATATTGTGGAGCCCCGGTAATTAAAGTGGGTTTGTTTATTTGGCTCCTTATAAATAAAAAGGGCT TAATACAGACTAATTACACAGAATCTTTCGTCTACGGTGGGATAATAATGGCAGCAATTTAAAGTGATATTAAATGA GAATTAATAAAGGAATATTGAGTCACTTTGACTGTATTAAGCTGTGAAATGATTCCTCATCTCAATTTAACTTTCTG TCTTGAGCATAATGTTTGATAAGCAATGCATTTTTGTAGGAATAATCTATAACAGTATTTGACTGCAATATTTAATA ACAACTAGACTTGTGACTTTATGTTGTTTTTAAATGGGCTGGGAAAGCCCGTTTTTTCTCTGCAAATTTATAGGACA ACTTAGAGGGTTCTTGCCCTATGGTAGCTTCACAAGAGTTCTGAGTTGGCACCCTCCGGTTTGGGAGGTAGGTGGAG ATGGCTTTGACTTTTGTTCAACTTACAAGGGAGTAACTTCTTGGAAGTGCAGTCCGGGCCAGCAGAAATGGTCATAG TAGGCCATGGTGGAAGGAGAATCAGAACCACGCGTAAATCCTCAGAAGGAAGAGGGCAGTCATTTTTTTTTTTTTCT CTTGGAGGAATATCCCGGGAAGAATTACCTGGAAGAAATCAGAGTGGTTTTGGAAGATGGATTGTCGGATAACATTT TAACTTAAGGGCTGAAGTTCCTTTCTACCTAAGTACCATTGAAGAAGTATGTTCCTGATCTTAAACATTTTTAATAG CAGTCTAGGCCAATTGACTCTGTGACCATTAAGTAATTTTCAAGTGGACTGGCTGCTGCCAGGGTGTTTGTGTTTTG CTTTGTTTTGTTTGCAGAGTAACTTGTGTGTAATTTCTTTCCACTGTGCCGCACAGCATGTCATGTCCACGTTAGCA TTTCTGCCCTTTTAAAGGGTCCTGCTGAAGGTCAGGACCTCACACTGAACACCCTTTTTATATATATTACAAATTGC TGGAATTGTTCATTAATTTCTGTCTTTGAAATGCCATGTCAACCCTCTAAAGGAATTCGCAGTGCCAGTTTCCCTTG GACAGCTGCTCACCATGGTCCAGTGCGAATTTCTGATGAAATTACAGGTGCAGGGAAATGCAGAATTAGAAATATAT CTGATGTTGCAATACTTGTGAAGCTATAGCTTTGTTGATGACAGGACAATTCCATTTTTGACAGTTTAATTTACTGT TGCCAGTGGACAAGAGCAAGAGGAGGACGTGGGGTTAGCCACCTGCCCCCTCGTACCGTGTTTCACATGCGAGGTTG CCAAATCATGACTCCCTCAACCTGTCGGGCTCAAATGAGAAGGTTTTCAGCTGGGAAAATGGAATATATCAGTGCAT GTAATATTGCCTGACCTCATAAAACAAAAGGGAAAATTGCAGATTCTGACAGCCATTATGAATCTTGTATCAGCATC CAAAACGACTTGGAGCCAGGGTATAGTACATGTTTATAATAGAAAAAAAACGGGGTTGCCACGTTGACTTGCAATAT GGAGCAAAAAATGACTTGTCATAATTCGTTCTCAGATGTTAAAGAGGCTGTTGAATTTACTAGTATATACACAGAAG AATGCGTAGCCCGGAGCAGAGTGTGGAAATAGTCCCTACACAGAGGGGTCTGCTGAAGTTAGTCATCGAGTTAGATT AGAATGGAAGGCAGGAGGGGAGGAACTCCGGGTTTGCGCCGCAGCTGCGTCGGTGGGGCCAGATTTCTCATTACAAG ATGTACTGAACCTTTCCACTTTCACCCACTGTAAATCTATAAGTAATCAGGCTTCTAAAATAAAGCAAACAACCTTG TTTTGAGATTATGAGAATCATTTGAATTGAATGTATTCTGTTGGAAAACTGGTGTATTGAGGCATGGCTTTTTATCT TACGCTTAAAAAGTTTTATTTATATTAAAATAAGGCATAATCTCTCTGCGTATAATTATACGCTCTTCGATTTAGTT TTTCTATTTAATAGTCTATTCCTTCACCTTCTTGCCCATTTCTTTATGTTATTCCTTAAAAAATAATTTCTAAGTAC AATAATTGTCTCAGCTGCTTTTGGTGATTTCTTTGGGAGGGGAGATGTGGAGTGACAGTCCAGGAACTTTCTGCCCA GGGGCAGGAGGCCCAGCGCGTGCTCCCCAGCTGAGGCCCCGTCCGCCTTGGCCACTCCCTTTCATCTCCCGGGCTGA ATGCGGAGTGTGGTGACTTTTATTTTTTCCTTTGCTATTGGTTTAGAATGAGATGTAGCAGTTGATTTAAGTGAAGT AAAGCATGCTTACGCAGTACACTGTGTCCCTGTCGGGGCCTGCTCTCTCATGTCACCTGCCGGCGGTGGCATGGAGA CAAAGCCTTCTCTCCTCTCTTACTGCTGCTGGGCCTCTCCTGGGCAGGTCCGGGCTCAGTGGCTTGAACAGGTGATG CAGGGAGGGGCAGTGCCTGTGGCCACAGTGATGGCACAGGTGGACATGATCCTGAGAGTCCAGTGGGGGCTGTAGCA CAGGAAAGGAGGTTGTCTACAGCCTTTCTCACCACACCTTTCCCTCCTCAGTGGAATCTCTGGCCCCATCTGGAGTC CCTGGAGGCTGGATGGTAGAACTGGAATCGTTGGCCCGATCTGGAGCCCCTGGAGGCTGGGTGGTAGAAGTGGAGTC TCTGGCCCCATCCGGATGATGGAGGTGGAATCTCTGGCCCCATCCGGATGATAGAGGTGGAATCTCTGGCCCCATCT GGAGCCCCTGGAGGCTGGATGGTAGAAGTGAAATCTCTGGCCCCATCTGGAGTCCCTGGAGGCTGGATGGTGGAGGT TTGCTGGGGTAACCCTAGGCGGTAGGTGTTGCCACCCAAGGTGAAGAGGAACTCACAGCAGCGCTGGCTTAGCAGCT CTGTGGGGCATAACCCACTTCTTTGCAAACTCTGGTGCTGGGAGATGATGAAATGAGCACCTGGTACATTAGGTTTG GGACTTAAAGCTGATACTTCCATTCGGGAGCAGGATGAGTAAGACCTGGTGCTTCTTTGCTCAGAGCTCTCTGAGGT GGGAGGCTGGAAGCGCTTCCCTGAACACTGTAGGAAGTGTTGCAGGAGAGCGGAAGAGCAGGAGGAGGAATGGTGCA GAGGAGGAGGAGCAGTGCGGAGATTTCACCTGGGGGGACACCACCACGGGAGGCAGGCGGCAATGGTGGAGGCGTTG GCTGAGCAGGGCCTTTAGAAAAAGGCCCGGGAGCTTGGCAGCAGAAAGGAGGGTGGAAGTGCAGAAGCATCGAGATG CCCAGTGACGTCTGTGCAGTTTGGATTTTGCTGTTGAAAAGCAAAGGAGATGAATAGAAAGCTTTAGGCAGACACTG AGAGTTTTTGCTTTGGAGGTATTTTGATATATGGTAAACTATTGTTTGGATTATTTTGGAGTATGCTTTGGTCTATT ATTAAATATCCCAGTCAAAGATAGCAGATATTCAATATTTGAGTTAGGATTTTTCAAATACTTAGAATATTTGAAAA TATTTTTATCATTAGAATTTATTGATTTTTTGAGACACTATCATATTCTAACAATTGCACCTTATTCTTTGGCGGTA TCACTGTCATTGTATTAATGGCAAAGGCTTCAAAATAGCCTGTCAGGCCCATGTCATCTGCAAGTCAACCCTCAACC CTTTCTCTCTAAACTCCTCTCCTCCAGTTTGCCAAACCACTGTGCTCCAGCCATGCTGCCCTCCTTGCTGTTCTGGA ACTTGCCAAGTATGCCCCACCCCAGGGCCTTGGCATGTGCCTGGAACACTTGAGCCCCCACCCTGCACATGGCTGGC CCCTTGTTTTCTTCAGCTCTCCGTCCATATGCAATAGCGATTCCTCATGCCCCTCATAGCACTTCTGGACTTTTTCC TTGCTCTATTTTTTACCAACTAAAACAGGGGTTGGCCAACCATGGCTCTCAGGCTGGCCACCTGTTTTGCAAATACA ATTTTATTGGAACACACTTATAAAGACTTGATTTCTTATGGCTGTTTTCCCTATGATGGCAGAGTTGAATAGTTGCA ATAAAGTCTGACCTGCAAATCTAAAATGTGTGTAATATCTGGCTCATTAGGCAAAAGTTTGCAAAGCCCTCATCTAG GATATAGTGCATTCATTTGCCTCTGTCCCTGACTGGAATGCACCCTCCCTTAAGACAGAGCCTATCTCTGTTTGGCC CACTGCTGTATCCCTAGAGCCTACAACAGTGTCTAGCACAAAGTATGTTCTCGTGAGATACTTGAATGAATAAATGA ACAAAAGTGAAAGTTCCAGCCAACAGGATGATGGGCAGTAGTTAACTATGCCTCTAATTCTGGCTTAAACTTTGGTA TTTCAGCCTTTATGTATGTTCTATTTTGCAGTTTTTATGGTGCACAGGATGAAAGAACACATCAACTCCTAGGGAGT AGAGGAGGCCTGGAAATAGACTCAGATGCAGAATTGTGTGCAGGTGGCTGTTGGGGAGTGTATTCATTCATTCTCAC ACTGCTATTAGCATCTGAGACTGGGTAATTTATGAAAGAAAGAGGTTTAATTAACTCAGAGTTATCTGGGCTGGACA GACTTCTGCTTCTGGAGAGGCCTCAGGAAACTTACAATCATGGCCGAAAGCAAAGGGGGAAGCAAACACATCCTCAT GGATGGAGCAGGAAGAAGAGAGAGAGTGAAGGGGGAGGTGCCACACACTTTCAAACAACCAGATCTCTTGAGAACTC ACTCACTATCATGAGAATAGCAAGGGGGAAATCTGCCCCTGTGATCCAGTCACCTCCTACCAGGACCCTTCTCCAAG ACTGAAGATCACAAGTCAACATGCAGTTTGGGTGTGGACACAGAGCCAAACCATATCAGGGAGTATTCCCAGGATAC ACTCTGTAAGGAAGTGCATACTGCAGGATCGGGCAAGGGAGAAGCAGGGGCTTGCATTATGACCTAGTTGCAGTTGA GGTCTCATCCTGTCCTGCAGGGAACTCTGGAGAGAGAATGACCTGTAGAGTCATCCCAAATTGAGGCCGGGGGCCTA AGTCTTGGCTCCCGGGTGCGCTCTGGGGGGGCCATAACCTTGGATGAGATGCTTCTCCCTCGAGAGCAGCGGTGAAG TATGCACCTGCCTCTGAGCTCCTAGAGCTGGGGTGTGATGCACTGGTCCTGAAACAAGGCCTGGGCAAGTACCCAGA ATTTCAGTTGTCAATGGAAAGAGTCAAACTCTGTAAAATACTTATAGGGATTTATTCTGAGCCAAATATGAGTGACC ATGGCCAGTGACACAGCCCTCAGGAGATCCTGAGAACATGTGCCTAAGGTGGTCGGGGTGCAGCTTGGTTTTATACA TTTAGGGAGACATGAGACATCAATCAAATACATTAAGAAATACATTGGTTTGGTCCAGAAAGGCGGGACACCTTGAA GTCCAGGGGACTTCCAGCTTATAGGTAGATTTAAAAATCTCCTGGTTGATAATTGGTTGAGTTTCTCTAAAGACCTG AGATCAATAGAAAGGAATGTCTGGGTTAAGATAAAGGATTGTGGAGATGCAAGTTCTTATTTGTGGAGGAAGCCTTC AGGTAGCAGGCTTCAGAGAGAATAACTTGTAAAATGTTTCTTATCAGACTTCAAGTCTGTGTGGATGTTAGTGCCGG AGAGGTATAGCGAAGCATATCCAACCCCCACTTCCCGTCATGCCTGAAACAGTCTCTCAGGTTACATTTTGAAAGAG CCCTGGCTGCGGAGGAAGTCCATTTGATGGTTGGGGTTAGAATTTTATATTTGGTTTACAAAGTCTTGAATTAAAAT ATTTTATCATTACCTAGTTTTATACCCGGGAATGTTAGTTGTTGCACGTGAGGACATGAAGTGAGTTAATGCAGGGC TTGGGCCGTCCAGTGTCTTCTTTCCCCACAGCCTTGCTGGCAGTGACCCCATATAAATAGGAACAATTACCGGAAGA AACTGTGCTCCTCCTAAAGCCTAGCTAGTGACTTCCACCTTTGAACCATAGGCGAGGCAGCACTTAACCAGGGCACT GTTTGATAGGATGTTACAGGTGGTTCCAGAGAAAAACGAAACATGTCATCTTGTTAAGGGAGGTCTTCGTTTCGGCA TCTCTGTAAAATATTTAAAGAGGTATTTTTATTTCTTTAAACAGGCTCTGCCTCTTCCGTCCCCCTCGCCCAATGTG GACTCTGCCCGTCCTTCCTGAGCAGTGAGACTCTGTCCCCATGTGCTTCTGGTCACAGCTGCGGTGACATCCTGTAA CATTGTTTGGTCCTTGCTTAAATCAGTTACTCACCATGTTATAGCCGAGTTCAGACCCACTACACAGACCTCCTAAG GTGCTGGCCTGTCTAAAACATTATTTCCATTATTCATTCCTGTTCACTCTGGGATAGGGAAGCCATGCTCCAAGCTG TATTGGAATATCAGGTCCAGGGTAATTAGCCAGGGCCTAAAGGGTCTGTTTTGGAAAATTCAAGTACAAGTAGAATC TTCTTTAGTATCTTGGATCCACCAGGGAAGAGATGGTCTCTGAGGCCTGCTATGCAGAATAAAACCAAGAGAGTCAC TGTGAAGGAATCCAAGAGATGCAAGTGCTCTTTGCTGTCCAGTGATTAAAATGAAAGCAAAGACGGGAAGCGTCCTT GATCCACATTTTGCCTGTGATTTGAATATCACATGCTTGGTCCGGGCACTTAGGTGGGCTTCAGTGATTTGGGCCGG AAGGAAATGGTGCCCCTTTCCTCGAGTTTCTAGATTGTTTTGTCACAGACAGGGGAAGCCCTCATCTGACTTGCCCT GTAGGACCCTAGTCTGTCGGGAGGTCTCTGTCCCCACCCCTCCCTGGAGTCTGGGGTGTAGTTGGGCTGACCCTCAA AATATGCTGTGTCATCAAAGGTTCATCATCTATCTGGTCTGTTTTTTAACGCAACTTGATAAAATACCAGTGTTTTA ATAAAAAGGTCTAAAACATTTACCATTTAAATGAGCCTCCCTTCCTGGGATGCACACAGGAGTCTTGGCGGGAGGCT GGTGGCGCAGGCTGATGGGTCCTGTCCCTCTGTGCCTTGGGCGCCTCTGCCCAGCGTCACGCAGTGTCATGAGACCC CTCAGTGCAAGCGCAGGTCTGCCTGAGGTCCCCCCTCCCACCTGCAACCTGGATGCCTTTTGTGCCTATGGAACACA ACTGAAAGGGCCGGTCAGTGCCATGGCTTGGCTAATTCATGGTGGACATGCATTTTGGTTTTTAAAATTATGGTAAA ATATACATAAAATTTCCCACCTTCACCATTTTTAAGAGTCCAGCTCAGCAGCTGTCAGTGCATTCACACCGTCGTGC AGCCATCACCACCACCCATCCTCAGAGCCCCTCATCTTCCCACAGGGAAACTCCATCCCCATTGAGCACAGACTCCC CCTTCCCCAGCCCTGGCACCCACCCTTCTACTGTCTCTATGAGTCTGACTACATCACATAAGTGGTACAGCCATTGT CTTTTGTGACTGGCTTATTTCATGGAGTATCATGTCCTCAAGGTCCATCCGTGTTGTAGCACGTGTCAAAAGGTCCT TCCTTCCTCAGGCTGGATATGGGCTCTTTAAAAGCCTATTTTCTTAGAGCAAAACCCGTTTCCCCCACACCTGATCG TCACAAGCCGTCTAAGACACGTGGTCTAGGGTTTGCTAGTGATCGTCTGTGGGCTTTCCGAGCCTGCGCTGGTTTTG CTGCCACAACCTGAGGTCCGCGGACGTCCCTGTGCTCGCTATGACTTCTTTGTGTGAGACTGTCAGAGTGTGTTCCT GGCCCTGACACGTGCTCGGCGGCTGGTTTTTCTCCTTCAGATCAGCGGCCTTGGACAGCTTTCGCTTCTTTGGCAGC CGTTTCAAGCTGCTCCGTCTTGTCATTTTAGAGTTCAATATCTGCTGCTCTGAAGGAAGAAACGGGGATGGGCTGGA CCATTGCACTTGGTCTCTTGACCTCGATGTTGCCCATGAGCAGTGAAGGATGACTTTTGACGAGCCATGTCAGATGT GACAGGCAATCTAGTTGGCTTGAGTGGAAATGTGCTGTACAAGTTTGAAGCCTTGGTTGGGGGCATTTTTCTGGTGT TCAATGCATTTAAATTGACACCAGACTTCAGTATGTTGTTCATTTCAGGCCCAGCACTGTAGGCTCCCATCTTTAAA ATGTGTGGGTGCCGAGCGCTCCCTCGTCAGCTTTTGCAAAGCATTTTGCATATTCCCAGTACTGTATTTACATTCCT GGCTCACATTGGTTTTTGTTTTTTGTTTTTCCTATCTTAAAGCTTATTTTTGAAGTGAATTTTTAATGCTTTCAGTT GAGTAGGGATTTGTGGAATTTTGAGGGGTGGGGAGGTGCAGTGGAGAAAAGGGGGAAAATGGTTGTTTCTTGGCTCT CAGCATAAGCTACATTTTCCATCATATGGAGTGAGATGCCTATATTGGGCAGCTGTGATTTTAAGTGAAAGTCATTT CCAGTTGTTTCTAATGTCTAGAAATTAGGTTTGTTTTGATTTTAATTGTCAAAGGAAGGGACTGTGGGTGAGGAAGA GCCCTTGCCGCCGCCTCCCCTGACGTGCCCCTGCTTGGGCAGACGCCTCCCTGGTGAGGTGTGGGCTGCGCTGGACA GCACCCAGGACCACGGCCTCGTTCTTTTCCTTCTCACTGTGTTTTCCTCTTGGGTAGGGGTGAGGGTGGTTATTTTA CTCAGATCTCTGAGCCTTAAAGTGGATCTTCTGCAGATCATCTCCAGAGTCATCTGACGGCGCCGGGCAGAAGTCTG TATGTTGGGAGCTGTCCCCAGCTCCTCTCCTTAGGACTTCTGAATCTGCTTCAACCAAGCATATTCCTGCCTCATCC GGAACATTCCTCACCCTCTGCCCCAGTGCAGGCGGTGTCCACATCTCAGGGATGCAGCGTGGTGGAATTTTTAAGGT TTTGGTTGGCGTGGATGAGAGCATCTCTGCTGAGGCTGCCGTGCGGGTGTGGGCTGGGTGCATCTGTGTCTCTGGGC ACCATATACCAAGTGGCACTCATCCCTGTGAGAACATGGCCTGGGCATTAGAGGATAGGATCCTTAGTGTGAGGGAG GCCTCAACCATAAAGGGACTGAGGGGAGAGACAGGAAGGGTCCAGGTATAAGAAGAGCTGCCCACGGCAGGGGGTGT GGCAGTGGGACCAGGATGAATGGGTCTCAGTAATGTGGCTCCTTCCCCAAGCTGGGGAGCAAGGGAGGCTTCTGCTG TTGGGTGTTGAAGAGGAATCTTTGGTTTGCAGATGGGGGTGGGGTGGGTCTTTGCCCATCCTCTGATCGTATGTGTA GAAGTTTGCAACATGAAGGGCAGACTGTGGTGGAGTTCTTTGGCAGTTAGGAAAGCAAGCCAGGGCACAGACGTGTT CTCTGAAAGGCAGGGTGTGGGCACCACAGGGTCAGCCCTGCAAACAAGAGCACTGAGCAGAGGGAGCGGCGATTGGA TTGGAGAACGGAGGCTCCCACCTGGCTCACATACTCCTACATACATGCCACACGCATGCCACGGTGCTTAGGAATCT TGAAAGCTGCAGGCTCAGGGGAGCTGCGATGCTCATCGTGAAGATGCAGAGCCGGAGAGGCCGCCCAGGCAGGAAGG CCAGTGCTGGTCGCCTCCAGGTGGTCTCAGGATCCCAGAGCAGGAGGCAGCCATGTGGTGCTGAAGGGGAAGCGAGG GCATAGAAGCTGTGCCCTGGAGCTGGAATGGTAGACAGGGCTTTCTTAGTGCCAGGGCCTGCAAATTCCCAACAAGG GCTCACTGGAATCCTCCTCCCGCCATCCTCCCAGGCCCCCCATTGGCCGGCCAGGCCCCTGGATTACCTCTCTGGTG CCCTGTCCTCCTCCATGCTCTCCTCTAAGGGCCCTCACTGGCACCTTTGCCCTCCTTAACCAGGTAACACTAAACCA GGAGACATCTAGAATCCTGTTTGAGACATCTTCGAGAGTCACCTTGTTAACTAGACAGAAGTGGATCTCTACAGGTT AGAGCCTGGGGAACCTCTGTTTTCTTTGTGATATTCTCTAGTGAGTGTTCCAGGAAAGAACATGCCCCACTGTCTCC TTTCTACTTGCAGATAGCGGTGAGGTGGGAGAGGGGCCTGGGAGTGTGCTCTTCATTCTCCACCTCCTCCCCCGAGG AGGTGGTCATGGTCACTCGCTTGACGAGCCCAGACTCTGAGCTTCTGCCCCTTTATTGAGCAAATTCATAAAGTCAA CCATGCTCTTTTATTATGTAACTTGTTTTTTAGATTCAGGGGTACATGTGCATGTTTGTTGTATAGGTAACTTGCCT ATCACAGGGGTTTTGTGTGTGGATTATTTCTTCATCCACATGATAAGCACATGATAAGCATAGTACCCAATAGGTAG GTCTTCAATCCTCACCCTCCTCCCACCCTCAAGTAGGCACCGTTGTCTGCTGTTCCCTTCTTTGTGGCCATGTGTAC TCAGTGGTTAGCACCCACCTCTAAGTGAGAACACATCCATGTTGCTGCAAAGGACAACATGGATGCAAATGATCTCA TTCTTTTATTATGGCTGCATAGTATTCTGTGGTGTATATTTACCACATGTTCTTTATCCAGTCTCCTATTGATGGGC CTTTAGGTTTAGTCCATGTCTTTACTATTATGAATAGTGCTGCAGTGAATATACGCGTGCATGTATCTTTATGGTAG AACCGTTTATATTCCTTTGGGTGTGTGCCCAATAATAGGATTGTGGATTGAACAGTAATTGTGTTTTAAGTTCTTTG AGAAGTCACCAAACTGCTTTCCACAGTGGCTGAACTAATTTACATTCCCACCAAGAGCGTATAAGCATTCCGTTTTC TCTACAGCCTCACCGGCATCTGTTATTTTTTGACTTTTTAATAAAAACCATTCTGACTAGTGTGAGATGGTGTCTCA CTGTGGTTTTGATTTGCATTTCTCTAATGATTAGTGATGTTGAGCATTTTTTTCATATGCTTGTTGGCCATATGTAT GTCTTCTTTTGAAAAGTGTTTGTTCATGTCCTTTGCCCACTTTTTTTTTACAGAGTTGATTGTTTTTTGCTTGTTAA TTTGTTTAAGTTCTTTATGGTTTCTGGGTATTAGACCTTTGGGGGATGCATAGTCTGCAAAGTGTGCTTCTTATGCA AGCACCCAGCCTTCCGGCACTGGGACGGGCCACATGGGAGGAGCCAAAGGATGGGAGAGTTTACCCCCTCCAGCCCA TGAACTTGCTGTGAGGACCCCTCCTGCCATATACCCTCCTGGGTGTGAAGAGGCCTCCTGTGTGGGCTGCACCACTG TCTCCGGGATTCTTGGCCTGGAGGCATCTCTGAAATTTCTCAGAATGCCCAATTTGCACCAGAGATAGAGCCAGGCC CCTTGGGATGGAAAAGGAAAAGAAACTAGATTTACTTTTTAAAGTTTCTAGAACTTCTTTGGTATCCCTAGAAAACT TTTTTTAAAAAGGATTTTCTTTTTAAGTTGGATTTCTATCCATTATTTACCTGAGACATAATTTCAGCTGATCAAAG TTTAGAGTGCTGGCAAGTGCCAAGCCTTACGATAGATGTTGTACCTTTTGACAAATGACCATGTATGCACAGCTCTG TTTCTGGACTCTGTTCTATCCATTTTTCTGCTTGTCCTCATGCCAACATGGCCCCGACTTCATTGCTGTGGCTCTGT AGTAAGTCTTGGTGTCTTGTAGAAGTTCTCCAAATTTCGTTGTTTTTTCCACAATTGTTTTGGCTCTTCTTAGTCCT TTGCATTTCCATGTAGAATAAGTGGATTAATTAGTATAGAAAAACCTGTGATTTTCATTCAACTGCATTCAATCTAT AGATCTATTTGCAGAGATATTTCACAGTTTTGAGTCTTGATCTTTGATTGTGGCGTATAGCTCCATATTTGTTTTCT TTGATACATCTCAACATGGTTTTGTAATTTTTTGGTATACAAATCTTTTAAATATATTCCACATAAATTTATCCATA AGTTTTTCATGTGTTTTTGTTTTGTCCTTTAAAAATGCTATATTTTTAGTTTCAATTCCCAGTATTTCTTTGCTAAT GATGAGAATTACAGGAGCTTTCTGTAAATTTGTCTAGTATGCTGTGAGTTTGCTGAACAAATTTATTCTAGTTCTAG TTGCCCAAGGATTCTTTAGTATCTTCCATGTACGTGAATATCTTGTTTGAGAATGAAGACAGTTTTGTTTCTTCCTT TATACTATGTATGCTGTTAGTTTCTTTTTCTTACTTTATTGTACTGGCTAGGGTCTCCATACTGCATTCAACAGGAG TTGGGGAAGTAAACATCCTCTGTTCCTAATCTTAGGGGTGAAGCACTCAGTATTTTACTGTTAAATATAAATACACA TTCTTGTAAATTCCCATTGAAGTTCTATTCTTGTAGGCTGAGAGTTTTATTACGGAAGGGTGTGTCATTTTGTCAGG TGCCTTTTTGCATCTGTTGTAATGTTTATGTGGTTTCTGTCTTTTATTCTGATCATATGGAGAAATGCAGTAATTGA TTTTCTAATGCTCAACAAACCTTGCTTGTCTTTGATAAGTGCCACTTGCTTTGAGAAATGCATGCATACATCTCTCT GTATATATATGTGTATAATTCCAGCATTATTTCTATATACATAGAAATAATAAATATACAAATAATGCTGGTTATAT TTGCTAATATTTTACGAATTACGGGATACTGGTCTGCAGTTTTCACATTTTGTCTTTTTTAGGCTTTATTATCGGGG TAATGCTGGCTTCATAAAATGAACTGATGTATTCTCTCTTGTACTGCTTTTTAAAATTGTTTAGGATTGGTATTTTT TTTATTTAAGTGTTTAAAAACATCTGGGCCTGGAATTTTCTTTGTGGGAAGGTTATTACTTAATTTAAAAAATATAT GCAATACTATTAAGATTTTGAATTTCTTCATGACTCAGTTTTGGTTATCTGTCTTTCAAAGAGTTTGTTTTATCTAA GTGGTTGAGTTTATTGGCATTAATTGTTCGTAATCTTCTCTTATCCAGTTATCCCTCTCAGGTCTCTAGGGCACGCC ATGATGATGCTATTCCACTGCCCTTTTTTCCATTCCTGGAATGGTACTTCCCACCTTCTAAATCAGTCTTGTTACAG GTTTATTCATTTTCTCAGTTCTGTATTTTATTGATTTTTATTTTTTTGTTATTTTGTGTATTTTACTCTCTTTCTAC TTTCTAAGATAGAAGCAGTGGGTCCCACTGAAGAAAAGCAGATAGCAGGCACTAAATTTCCCTGTAGGGTTAACTAT GTCAGCCTGACAGACCCCAGGCTGTGTGTCTTTGGAGGACAGAGACATTGGAGGCCGTTCATGATCCTTTCTAAGAT TGGTCATTGATAGGAAACTTTGTTTGTTAAGTAATATAAGTATTCAAAACTAGACATTAACTTCTAAGCAGTGCTTA AGCAGCATTCTACATGTTTTGATAGGGATTGTTTTTTAATATCACTCTGTTAAATATTTATGTACTGTAATTATTGT AAATGGTGATTACACTGTTCCATGTGCATTTGCGAGAATATACCCTGCCATTGTGTGCAGTCTTCTATAAATGTACG TTGGGTCAGGTTGTTTTGGCGTGTTGTGCAGGTCTTCCCTATCTTCTGACTTTGTCTCCCTGTTCAATCAGTCGAGA AAGGAGTGTCGAAGTCTGCAACTCGAATTATGGATTTGTCTCTTTTCCCCTTTGGTTCTGTCAACTCTTGCCTTGTG CCTTCTGTACATTTGCTAGTAGGTGCCTAATCACATGAGATCATGACGTCCTCCTGATGTGCTGGCTCCTTAACAGT ATGAAATGTACTCTTTATCTCCAGCCATTTTTCTTACTCTGAAGTTACTTTGATATTAATATTGCTAGCTTTCTTGT GATTTGCCTATGCATGTGTTATACTCTTTTCCATCCTTTTGCTTTTGATCTATTTCTTTATATTTATAGTGTGTTTC TTGGAACCAGGATATAGTGATCCAGTATGCAAGTCTGTACTTTTTATTTGAAATGTTTTAGTTAATGTACTCATCAA TAGGTATGGGTTTAGTCTGCCGTTTTGCTGATTTTTTCATTTGTACTTTTTATTCTTCATTCCTGTTTTCCTAATTC CCTGCCTTCTTTTGGATTGAATCCTTTTTTGATTTCATTTTATAATTACTATTGCGTTATTAGTGCTACCTTTTTTT AAGTAGTTTGTTTAGTACAGTACTTTTTGTTAGAAATTCCAGAAATGATGGAATGCGCTGTATCCACACTGTCCAGT ATGGCAGGCACTGGCCACCTGTAGCTGTTGAGCACTTGACATGTGACTCGGGCAACTGAAGGAACTGGGTATGCAAT TACGTTTAATTTTAAATAATTTAAATATAAATAGCCACTTGGGGATACCTGCTACCACACTGGGCAGGGTGACTGTA GGGTTTCTAATATTATTGTTAACTTCTCATGATCTACATTGAAAATGGTGTTCTGTGACGTCACATGCAATGCAAGA CCTTCACCACAGTGAACTTCCATTTGCCCCTTGAAGTCCCATGTACACGGCCATTGATCTTACTTCTACATGTTATA GACTCTCAAATACATGGTTGTGACTTTTTCCTTTAAACAGTCATTTGTCGAGAAATAATCATTTAGAATTGAAATTG AAAGTGAGAAAGTACTCTTTTCACGTACGCATTTTGATGTTCTAAATATGTTGAAAGGGCGCAGGGGGGTTTAATGC CCTTCATCAGATCTGTGCTACCTTGTGGACAGTAATTGGAAAGGATGGGGAACAGGGACGCACGAGTGGGTAGATGA ATGCGAGGCTGTTGAAATGGTTCCATGGTGAGCAGTGCATGGTTGCTTGGAACAGGAAGGAGCGGAGGTGTTAAGAA GTGGTTGAATTTGGGATCTCTTTGGAAGTATGATGCTCCAGCTGTTATCAGCAGAGCTGTAAGTGAAATGTACTTTG ATTGTTTGTTGAACATCCTTGCCACCTTTCCTATCGAATTTGCTTTTCAAAAATAGTGTGGACATTAAAAAGCCATC CCCATGTGCCTAATTTCAATTAGAAAATGTTGAATCACTGTGAATTTGGTGAGTCATCCTGCATGTAAGTAGAGCTA TCCATCTAACTCTTAGAATTTGTTAACTATTAAGAGAGGAAGACAGAAGGTAATATAGAGATGACTATTTATGAGTC ATTTTATTTATTCATTATCCATCTAAAGAGTAGCAATTGATTGTATCACAGAACGGTGTTTGTTTTACATCTTTTGA TTAGGAGCAGTGGTTTATGGCATAGACTTGCTTAATTTCCTCCTGTTTCTTCCTCTGGAGTGGATAACTGTTTCACA ACAGAAGGCGATAAAAGATTTCTGGTTTATTCATTAATTTTGCTTTGAGACGGAATACCTGATCTCAAGGATAAGAT GCTGATTGGTATGACCTGAATCGAATGATTAGCAGTATGTTGCCCTTACTCTGATCTCCCCACAGTGTGAGAGATTT TCTCATCTTCGCATCAGTTTTTAGCCATGTTTTGGTGTTTAAGCAGAGATACGATTCCACTTTGAAAGCTCTGGAAG TAATTCTCAGATCACATATATTCATCTCAAATTTGAAATCTGGAGTGTTTGTCTTGAAATTAAATTGTGGTATTGTA AGCTGTCAGTGGCATGATCTGTGCTGGAACCTGATGCGGTCTAGTTTACACGGTATTGAATCATGTAAGTATCTCAT CTTTCCTCTTTGAATTCAGTATGACTTCACATAGGTGAGGTTAGCTTTTAAGTGTACACAGAGGTATGGTTAACTAT CAGCAGACTTCAGTTATCAATTGCTGATCTCAGAAAGCTAATTTATCTTTAGTGTCCAATAACATTACATGGCTTTC TTTCTTTGTTTTATGTTTTGTGTATATGTGCTTAACTGTTTAAATAAGTTCTGGATTGCATCCAAATAAGGTTTGAT ATGCTCTCATTAAGAATATGATATTGTGAATAAATGTAATCCTGACTTTCAATAAATGGCCTGTAAAAAAAAAAAGA AAGTATTTAGAGTTAGTGAGTTGAGGGGCTTCGTCACATGGCTAAAGGAGATTTGAGTAGCCCCTGGAATGTTGAGT GACTCTGACTTGTGGAATGCATGGAGCCCCGTTTCGTTGGCTGGCAGGATCTACGTGGGCCGAGCATACAGCCCTAG ATGAATGACTTATGTCTCTGAATTCTGTTCCTAATCTCTGCTGCTCAATCTGTGGCCACCTGCAGAGGAGCCAGCCG GATGGCATCGTCCTTACCGAGCTCTCTGGTTGTCAGATCCTTCACTACTCCTTAGTGGCTGATTACTCTGCGTAGTG TAGGGTGCTGTAGGGTGCATAACTGATTAGTGCCTTGCAGATATAAGCAAAGTTTACAATGAAGCCCTTGCGCTTAG GAGCATACAGTCTAGTTATGAAGAGAAGACACAAAACACCCAGTCACATCACTGTGAACAGACAAGTTTGCCAGGAG TCTGTTCAGACCTGGTGCTCCCTGAGCCGCGCTGAGCAGCAGTTCAGCGGGGATACCAAGTTGGGGAAGATGCCAAG TGGTAGGAAGGAGGCTGGGGCTCTGGTCTGCACATGCCAGCTGATGGAGTTTGGAGTTGGGGGGATATGGCCAGTTC CCAATGCACTGGACCAGCCACCATCCAGAATTAGGAAGTAGATCAGCCCGTGCCTGGCATGCATTGCTTTCTTTTCA GTTTCTAACATATATATAACCTTTTTAAGTATATATATATGTATGTATATATATAACTTTAGTTATAGAGTCTCGCT GTTTTGCCCAGGATGGAGTACAGTGGTGCAATCATAGCTTGCTGTAGCCTCAAGTGATCCCTCCCACCTCAGCCTCC CAAGTAGCTGGGACTACATACGTGCACCACCACGCCTGGCTAATTTTTTTGAAAGGACAAGTCTTGCTATGTTGCCC AGACTAGTCACCAACTCCTGGTCTCAAGCAGTCTTCCTGCCTTGGCCTCCAAAGGAGTTAGGATTACAGGCATAAGC CACTGTGCCCAGCCTAATCTATATAACCTTTCAAACAGGTATCTCCATTTGTATACTAGGGGAGAATGTAGACCCCA CACCCTCTTCCTCAAGAAACAACTTGAACGCATGCCATTCTGCCATCTCAGCCAAGGCGATTGCCTGTGCCCTTCCC AAGGGCTTGGAGACACCCTCTCCCTGCTAGCCCCGTGCAGGGGTCTTCCCATGTGTACCCTGCAGCACCGTCCACCT GTCTGAGTTCGATGGGGCCTCCAGGCCCCCTGCCTTGGTGAGGCAACACGCTCAGCGCCTGCGCGAATCTTCTTGCC CATCAGTTCCTTTTCCTTTTATCCAGGATTTCACACACTGGGACATTTTGTGCCAACAATTATTAATCAGTTTTAAG ATTGACTGGGTTTGTGGACTGTGCCCAGGGTTTGTGCACCTCACTCACGGCAGATTTCCTGGCAGCCCTGGGGATGG TCCTGCTCCCCCGGGGACAGTTTCCATCTCATGGTGTAGCTTTGGGAGTGGGGCCCACTGAAGAAAAGCAGATAGCA GGCACTGAAGTTCCTGCAGCCTGTTGGACCCCGGGCCGTGTGTCTTTGGAGGACAGAGACATTGGCCTACAGTAAAA TAAAGTCAGATGAGAAAGGGTGGGGAGGCTTTCCTGCTGTAAAGGGATTATGTATTCATACTCGAAATAGGGATACA TTAGGGGAATAGATTGTAATCTCTACATCCCCAATCCATGAAAACAAAACAGAAATGGAGGGCCAAGAAACCTACAC AGGGAAGACACGAAATAGCAAACATACCTGATTTATCCAAGGGAAGGCAGAGGAGGGAAACGAAAGGATGAATAACA GGTGAGGCAAGCAGGACATAACTCGTAGGATGGTCAGCATGCCCAGTGCGGCGGCGAGGACTTCGGAAGTCAGTGGA CCCAGTGGAGACTCAGATGAAAATCAGAGATGGTCTCACTGTGGGAACATGGTGTGCAACCATAGACTAAGGCGGCA CACCTTAAACATAGCCTAAGAGATGCACCGTGCAGGGCAGGCACCCACATGAAGCGGCTATGTAAACGTGACAAAGT AGATATTAAGAAAAGTAGGAAGGAACAGTAAAAATAAGATCAGATATCAGCAAAGCAGAAAATGGTATTACAGAGAT GTTAACAAAGGCAGAAATTATGTCCTTGGAAAGAGTCGTAAAAATTTTCAAAATTCCCAGCCAAACCTGATGAAGAA AAATGAAATGACAAATAATATAAATGAAAAGGAAAGATCACTACAGAATCTACAAATATTCAAAGATAATGAGGATG TTATTTAAAACTTTATGTTGACAAATTTGACAGTTTAGATACAATGAGCTAAGTTTTTGTCAGAGCTTAACAATTTA ATCTAAAGAAATCAAAAATTGAAAATTAAAAATCTTCCCTCAAAGAAAAATCTAGGCCCATACAGCTTCACTGTTCA GTTTTATCAAATAGTTAAGGAAGAAGTAATTCCAATCTTATATTAGCCCTTTCAAGGAATAGCAGAAGAGAGAATAA TTCCTAGCTTGTTTCATAAGACCAACACAACCATGACACCAAGATGCCAAGATGTGTCAAGGATGTTTGAGAAATGG TAACCACAGACCAATGTCTCTCATGAACCCAAATTTTAAAATCCTAATAAAATATTAGAATGTCAATTTTACAATAT AGAAAAATATGTATATTTCCAGTGGGGTTTATTCCATTCAAAAATCAATCGGTGTAATTCTCCACATTCACAGAGGA AAAGAAAAGAACTTTATGATGATAATGATCATAGTAACCAAAAAAAGCATTTGACAAATTTCAACATCCATTCACGA TGAAAATGCACTGCAAAGTAAGAATATAAAGGAAATTCTCTTACATGACAGCGTGTATACGAGTGTGCACACGTGTG TGTGCGTGTGTGTGCATGCATGTATGTGTGTGTACACGTGTGCACCTGTGGTTTTCTCTCCATGTTTCTTCCAGTAC TGATCAGGGTTGCTTGTTTTCAGGAGTTTTCAAGTGCTCTACCTTCAAGTTTTCCCCAGCTAAATCTTTCCCATTTC TTTCTCTAAATCCCTTTTTGATATTTGCAGTTCTTTTCAACTTACCACCAATATTTTAGTCTTGATTAGAATCTTCC CAGAAAAGACAAGTTTTCCTTCTGATTCCCACATGTCCCTTCAGCTCGTGATGGCGCAGAATATGTTTGCTTGCCAG TTAGGGGAACCTGAGGCTGCCTTCCTGGGCAGAGTGGCGATTTGTACTGGGCCAACGGGGCAGCCACTGTTACTTAT GGACAGGTGGACTAGGCTCTTTTTCATCCTGAGGAACTGCTCCGCGCTTGGGAAGAGCCACTGGAGTTTATCAGCCT GCGTGTAAGCATGTGGGAAGGGGGATCAGGAACATTTAGTAGAATAGTCCGTGCTCCAGGTGATCAGTCCCCACCCA CCCGCATGCCTCTCCTTCTCCATCCCACCCGCATTTTGGGTCATTGGGGGATTAATTTATTCGTGTCTCGTAAGCAC TGGCAGAACACCACCCGTGTGAACAGCATTGGTGAAGCCTGGGGAGTTCTTTTTGAGACTTCCTTGAGCTTCCGGCA GCACGTGTGAGTTTCGGGGGTGATTGGAATGTTTCTCCCTGGAGGGAGGCCTGATGCCCTGGAGCTGGCTCCAGCCC GAGCCCCGCGGCATGTTCTGCCTGCTGCCGCCTACCTGCAGGGAGACGCCTGAGCTGGGAAGGTTCTGAGGGTTGGC AGGGTGCTCTGCTGTGCTCTTTGGGAGGATCCCCTTGGCTGTTCTGCATGCCCAGGGCCGTGCTCTCCGTGGAAATC TATGTTGGAGACGACAGGCTCCGGTTCCAGCTACACCAGCCTGACTTCCACTGGACAGTGCCATTTTCTGCCTCTGA TAACACCGTGGTGAAGCGCCTTACGTAAAGCAACAGATTTTTTGTTTTAAGATGCACTATAATTACTCTTTTCAGAA CCAATAATATTAACCCAGTGAAGTCAAAACTGAATTGTTTTATAAACTGACTATGGAGCTGGTGTAAGGAGATGCAG GGAAGGCTTCTCATCTGAGTCGGTAGCAGAGTACATAGTGCAAGAAGCGTGGGCAGGCTCCCAGCGCACCCAGAGAT CAAGGAGAGTGATGGGTGCTGCCCGCCTCTGAGGGGACGGAGGCAGCTGCCACCCGCCGTGTCCCTCACGCTGGCTT TCAGAAGGCTTCACGCAAGTGGGCTCACAGGCAAGAAGAAAAGCTGTGGGCCGTGTGTCTCCCTTGGCTATGGCTCA GTTTTAATGTAATTAGAAGCTCAACTTGAAAGTGAGATATCAAAGGAAGCCTTCAGTTGCTGAAGATGGGTGTGCTG GGGGATGAGGGCGTGGGTTGAAAAGTATCCAGTTACTGTTTTTCTCACGTGGATCAGTCTAACATATGTGACTCTGG TAATTGAAGATGGTGACAGGCTAAACTAAGCTGGGATTCCCCAGGGAGAGACTAGGATTTCTGGAACCAGTCTAACA AAAACAGAAACTTGAAGGAGAAAACAGAATATGCCTCCCTCTCTTCCCTGCTTCCCTCCTCTACTCAAAAAAAAGAA AGAAAGAAAAAAGCAGCTTTTGCAATGAAGAATTCAGTTTTTCGCCCCCGAGTGTATGATGGTAGTGACTTTGGTGT TCCTGGTGCAGACCTGTATTTTTTTGTGATGTCTTCATCTGGCCCCTCGCCCTCCACTCTTCATGATGAATATGAGA CCTGTGCACTGAGGGTCCTCACCAACACTTCCTTCCTCACAGCCGTATGATTTCATCCGGAAGCGGCCTCTAAAAAT AAACACCCTCATGTTAGCCTTTTTCCACCATGCTTCATATTTGGCTCTTGTATAAATATCTGCCACGTTTCCACTGG GAATTATTGCTGTTATATATTTTATAGACACTCGACTGTCATCTAAGTGGCTTATTTTATTCCCATAGCTAGAAGAA AAATACAAAAATAATTTTGTAATGTGAGTGTGATTGTGGGCCAGGAGTTATGTTCTGAGAAGGAGCTGAGAGTAAAT TATATTCCTGCTTTATTGTGTTGATATTGTCTTCCCTGTTGTGTCCTGAGTTAGCCTTTGTTGTTCCAAGTTATTAA CCAGAGGTGGCTTGATAATGACGCCACTTTGGAAGCGTCTGGCTGGTGTTTAAAACTCTAGCTGGTGATAAATATAT TTTAGTCCTATTAATGAAGAATTGTAACATTTTAGTCCTATTAATGAAGAATTTTAACATCCATGCAGATGAGCGGC ATCCTAAAAGCAGCTTCAGATGCTTTGGGGTGTTTTCTACCGAAGATGCTTTTGTAGAAATACCGCCTTCCTTTTCA TTTTTTACACTGGTCTGTTCATCAAAAACCATAACTTCATGGAACTTGCTTCTGTGGAATGAACTAAATGTAAATTA TTTTTCCAAAAGCTTTATGAGTTTATCATACATGTTTTGGAAAAAGTGGAAAAACACTAGGAAGAAGTTCCAGTGAG CAAATGTTTCCCGACATGTTTCACGTTTTGCTCTGGTTTGAAGCAGGGCGTTTTGTGGAAGGTAGGGCTGGTGGATC GCTCGGTCGCTGGGCAGTGCTGGTCACTGCCTTTCTTTGGTTGAGCTGGTCTTGAGTCCTGGTACCTGTGTCTGCCC TGGCATTGACCCCGTGGTGCTTTCTGTGCATCTCCGTGCTCACCTTTCTGGAACACAGCCCCTGAACAGAATCATTT GGCAAAACATCCCAGTACAAAAACCAGTGGGTTTTACATGGGGAAAGAACTCAATGAGTCCACAGTTCGTGTCGATG AAGGAGGCACTGCGTGTTGGGCTCAAACGCATCTGTAACTGTGGGTGCCAAGAGACAAGTAGAGCTCAGACTGCTGG GAGCGTTGGCTGGAATAAAGCTGGCCCTCACGGGGCTGTTAAAATACTTGAACTTGAATTCAAAGTCAGATTTAAGC TGACTTCACCCCTCTTCCCTTGCTTCTGAAGATTCTGGTTTTTCACGAGACAGAAAAAAGGGTGGAACCAGGAGCTT ATCCCCTTCCTTCCTTCCTTGCAAGCAGCAGCAACAGCCATTGAAACTCAAAGACAGCAAGAGCTCTCTTCCCGCCC ACTCCCAGCGCCACTCCCGGGGGAACCCATGTGGGAAGGGCTCTTCCTGACACCTCTCCTGCAGCACACAGCACACG TCAGCCTTGCCCTGCATGCAGCCTTCCCAGCTGGAGCAGGTCCCACCAGCGCCCTGCCAGGCGGCAATGGTCTACAC CGCCATTTTACCTTCCCCAGGTCTGTGGCATTAGGTCTTCCCTTGTGAGTGGCTCGTATTCTTTTCAAGATACTGTA AATAATTTGGCATCATATGGACCACTTGATGCTTCTATATGAAACATGCCTTATAAACCCTTAGTACTGCATGATTT CAGATTACGGAAATATCTGCATGAGTATTGGCTGCGATATCTCTGTACTAGAAATTTCAGTGCTACCTCTACACAAA CAGACACACTGGGGATGTGCTGTGTGTATTGTATGAGTGTATCATAGAATATTTCATTGTCCGTGGTAGTACATTTC ACATCATAGGCTGTCTGTGGCTACTTTTAAACCTTAGACATTTGAATTTTCCCTCAAGCTTTATTCTGGTGAAGCGT GGGATGGGGTCTTAGCTCACTTTGGAAGGATGGAACAGTTAAGCAGCGTACCTGGTGACTGGCACTGTGTGTGAGTG ATGCCACCTCGGCACCTTCGCGTTGATCATCTTTAGGATCTTTAGGATCACTGAGAATTAATCATTCTTTGCATGCA TAAACCTGTGTAAGGGTTGCTCCACGTTTCTGCCATTGCTCACAGATAAAACACATTCATGATCTCAGCTTAATCCC AAGAGGATGTTTCCCCATCCATCAAAACCACAGTGCAGGTTTTCACTGATGGGACATAGGGAAGGTATGGCAAGCTG GGTGGAGGTGTGTTTCCTGGACTTTTCCGGAATAACCCAACCCATCCCTCCTAGTGAGCACAGAATTACCCAGAAAG TTGAAAAGGAAAACACAGTTTATTGGAACAAATATGCAAAGTGATTTGTTCTTTTCACAGAGAAGGTAAATAGCATG TCAGCCGCCAAGGCCCTCACACACACGGATCTGCCCGTGGAGTGATGAACTTGTAAAAGAACGTCAGGTGGAAGCGG TGAGAGGGCCTGGACAGTTGTTCTGCAGCTGTGTTCTTTGTCTGGTTAATCGCGTGCGTCTCCAGTCTGTGGCTGGC CAGCCGCGTCCACCTGCGTGCTCCGGGCACCCAAGGTGCTTGGTTAATATGCAGCTCTCTCAACATCTCCCCCTTAG GAGCAACAGGTACCCGGTGCTGGTGTTGTCCGTCCAGATGAAAGGGCCTGGCCACTCTGATTGCACTTCTCTTTGAT CTCCCTGTAGAAGGTCGGCTTGTCTGCCTAGAGATTCCTCTGAAGGGCAATTTAAAAGTTGGATTTGAGCAACATCA AGTGAAATGTCTTCCCATCTCACGCCGTGAGGAGCTCTTTGGTGTGGCTCATGGCTGGATGTGAGGAAAGATATTAA CCCTTCCTTTGAAACAAACTGTCTCACAGGCTGAACTTGCCAGCTACCTTTCTCCTCTGCTTTTAGAAAAGAAAATA TTTTCTTTCTCATAATGCTCTTGGCCCTTGAAGATAAACTCATTGCAGTGTTTAAAATTTGTCCTGAGACCCTATTG CCTTTACTTTGTTAGTGTGTGTGAGACTGAAGGCCTGTGGATGGGTGAGATGCTTCGAACTGATAGAAAAAGAAAAC AGAAGGAAGATTTTTGTGGGCAGAGCCGAGACGCCGTCTCTCAAGGTGTTGTGATCAGTAAAAGAATCCATTTTTAA TGGTGAACTGGCAAAGGCTGCTAAATACCAATTACTGAGTAAAGTGCCATGAGAAGGTAAAGCCACTTTTTGTGAAT TATTTCATTGCACTAACGTGCTCCTACTGAAAAACATGGCCAGGCTGCTTCCGATACAGTTCACTTAAAGAAAAATG GCTCACGTTAAAAATAGTTGCTTTATTTTGTAAATGTTTTAGAAAAGTGCATTGCTGTCCAGAAATCTAGATGTACA CTTGTTTTGTGTTTAATGAAAAAGGGAAGGAAAAATATCTCGAAATCCCAAATCTCATCGGTATCAATAGGATCATA AAAGTAAAGGCAGGAAAAGGAATTTGCTGCTCGTGTTTCCCATCCATGTTTCCAGGTTATGTATTTTGTTTCTCAAC TTTTATTTATGTTAATGAAACCAGTCTGCTTTTTTTTTTTGTTATTTTTGATGACTAAGATCCCATTTATTAGGAAT TTGTAACTCAGCTGTGACAACGTAGCCTGAGATTTACACAGTCCCCGTTAAGGGTAGAAAATAGAGGGAGGGCTTAG CTCTCTGTTTTCATAGCTGTGTTAAAGAGCCAAATAGTAATTTGTTGACCGAATGTATTATCTGTCCTGCCATATGT TGTTCAAATGATGAGACAGAAGAATGAAGGACTCTTACAATGTAAACTTCTGGAAATCATACTGTTTCAGGGTTGTT TACATTAATAACAATGATGGCTTACATGTATGTATGGTTTTATAATTTTTTGAGATTATAAAAGGTATACATGGTCA GTGTTGAAAATAAAGTATAAAGAAATTAAAAATTTGGACGGTGTGGTGGCTCACGCCGGTAATCCCAACACTTTGGT AGGCCAAGGTGGGCAGATCGCTTGAGTGCAGGAGTTCAGGACCAGCCTGGGCAACAAGGCAAAACCCCGTCTCCACC AAAAATACAAAAAATTAGCTGGGTGAGATGGTGTGCACCTGTGGTCCCAGCTGCTCAGGAGGCTGAGGTAGGAAAAT CTCTTGAGCCTGGGAGTTGGAGGCTGCAATGAGCTGAGATCACAGCACTGTACCCCAGCCTGGATGACAGAGTGAGA CCCTGTCTCAATTTAATAAATAAATAAAATAAAAAATGTATATTTCTATTGCATAATTAACTATTATTTATGTTTTA TCAGTCCTTCTAATTTCTTTTTTTCTTAGGAGACAGGGGCTCCCTATGTTGCCCAGGTTGGTCTTGAACTCCTGGGC TGTAGCAGTCCTCCCACCTTGGCCTCCTGAAGTGCTGGGATTGCAGGCGTAAGCCACCGCGCCCAGCTTTCTAGTTT GTTTTTATGCCTACAGCGTACAAATGTTACTAAAGTTTGGATTGCGTCACACTGCATGTGTTGTAGTGGGATATGCC TTTTTATGCACCAGCGTATGCATTGTGCCACATCCTTAGACATGACTTTTATTGGATATGCAGTAGCCTGGCTTACG GATGTTTCATAATTTATTTAACACCCTGTGATTGGTTATTTAGCTTACTTTCATTTTTAACTGTACCAAATTGTGCT GTCATGCCATCCTGGAAGTGGATTGCATGTGGGCGCCTTGTACTTGATAGAGCACTTTCTTAAACTGTATCTCAGTC ATTAGCACAAGTGCTCCATGAGGCAGTGAGGCCACTGAGTAGTGATCCGGGTCACAAGGGAAGAACTGGGGCCCAGC GAGACTGAAGCGGGTATTTTCACGTGAGACTTCGCCAGTACGGGGCAAAAGCACACGGTAGAGGATTTTGAAATCAC CCTGATTGAAGACCAAGCTGGTTTTTGTCCCAACAATAGTGATTCCTTCATTTATCCCACAAATATTTAAATGCTGC GTACGTGTCAGGCACTGTCCTTGGTTCTGGTGAATAGAGCAGACCCTGCCTGCTGCTCCGGGGGATGTCGGCCCGAT TCTTTGGTAACTCTGGAAGGCTTGTGCACCTGCTGGAGTTTGGGGGAGAGAGGGAGGGTTGTGGACCCAGTCACATA CCACAGTGGGTTCAAAGTCTCATCTTTATTCTGGGAGTGACTGGGTGTCTTTAAAAGTCAAGTTCGGAAGATTCTCA TTTAATGATATTTGATTCCATGTCTCATGCAGAGGAATACTTAGATACGACGGAATGAGTTAAGTAAAGGGTAAAGT ATTACTTAAATATATCATCTGAAAATAACTGCTTCATATCTCTTGTATCTTAAAATATTTTCTGGACAAAAATTTTA TGTAAATTAAAACAAAATGCTGTTTTTCATCATTGTATTAGGAAAAAATGTAAATACAAAATATTTGGTGTTTTGAA GGATGGGCCGGAAATGGGGATGTTTTTGTATGATGCTGCTGATAACAGTAAGTTGCTATCAGTCTTGTGACCATATA CATACAGACATAAAAATGTTAAAACCCTTCTCAGTACTCCCACTTTGAAAAAGTATACTCTAGAAAAATATCTAAAT TATAGAAAAAACGTTATACCTTGCAGCTTCTCTTATCATGGTGGAAAGGAAACCAACCTAGACGTCCAACACTAGTG AGATCGTTTAGATAATACTGACACATGCTCTCACCAAACCGAGTCAGCATCTAATGCCTATGAGACTGCGTAGTCAG GGATAAATGCTTCCGTTAAGTGAAAAAGCAGAGTGCTAAAGAGCAGGCACATTAGAGTTACAACTGTGCCATGTGAG AAAAAGCTTGAAGTGAATACTCTCAAATGATGATAATGGCTGTTTTAGGGATGAAGATCAAGGAATTTCCTGTTTTT TCTTGTCTGTATTTCAAATTTTTTATAGTATAGTTATATTACTTTTTAAAACTAGCGACTTCATTTTATAAAATGAC TTTTACTTATTTTTAAAATTTAAGCAATACAGGAAAGCAAAAGAAATAACAATTACCTCCCATCTCACAGCCCCCAG ATAAGTAGTGCTGATACTTGGTGAGCCTCCTTGTAAAAGTCACTCTTTGCTTGCATAAACATGTGGTTATATGCATA AAAGACAGTAACATAAACACAGATAAAACGACAAATATTTTCAAATGACAATAGTTTTGTTTAATACAAGGTTCTTA AATATTGTCTGTAAAGAACCAGATAGTAAATATATTAGTCTCTGTCTCAGTTATTCTGCCGTTTTGTGACAGCAATC ATAGACAAAATGTAAATGAATTAATGCAGCCGTGTTCCAATAAAGTTTTATTGCTCTTCATTCTGTTTAGATCCATT TCCACCTGGCATCATTTTCTTCTTCCTGACAGTTTCCTTCACCCTTTCTCATTGCACTGGTCTACTGCAGGTGACTT CTTACAGTTTTTTAGTGCCCAAAATGTCTTTATTGCTTCTTGATTGTTGAAAGGTATTTTCACTGGGTGTAGAATTC TAGATTGACAGGGTTTTTTTCCTCCCAATACTTTACAGATTTTGCTACACTGCCCTCTTGCATGCATTGTTTCCATT AAGAATTCTGCTGTCATCCTCATCTTTTTGTGCCTCTGTGTGACATGATTTTTTCCTCTGGTAGGTTGTAAGGCCCC TTATCAATTTTTTGAACAATTTTATTGTGATGCATCTTGGTGCAGTTCTTCATGTGTCTTTTACTTGGTTTCACTGT CCATCTTGTATCTGTGCATTTGCAGTTTGCATCAAATTGGAAAATATTTTGGCTATTTCTTCAAATATATTTTTTTC CTTTTCCTTTCAGAAACTCCAACTACAGATATGTACGATTTCCTGAAGTTTTTCAAAACCTCACTGATAATCCTTCC TTCCTTTCTTCCTTCCTTTTGATTTCCTTTCTCTGTCCTCATTTTGTGTAGTTTCTATAGGAATATATTCAAGTTCA CTAATTTTTTCTTTTGCAGTGTCTAATCTATTATTTTCATTTGATGTATTTTTCCATTGTAGGTTTTTTTAGTTTTT AATTCTGAAAGTTCTCTTTGGATCTTTTTTTTACTATATTCTATAATTCTAATTAACTTTTTGAATGTCTGGAATAC AGTTACATTAACTTTTTGCATCCTCGTCTGCTAATCTTAACATCTGAGTTAGTTTTAGGTCCATTTCAATTGACTGG TGTATTTCTTCGTAATGGATCATATTTTCCTGCTTCTTTGTGTGCCTGGTAATCTTTGATTGCATGTCCAGTATTGT GAATTTTACCTTGTTGAGTTCTGGTTATTTCTGCATTCTGATAGATTTCCTTGAGCTTTGTTCTGGGATACAGTCAC AGTTAAATTACTTGGGAATAGTTTGACTCTTTTGGGTCTTGCTTTTAAGATTCGTTAGGTAGAACCAGAGCAGTGTA CTGAGGCCAGCCCCCTTTTCAGCTCGCCCCGGATGGTCTGTGAATAGGAGGCATTCCAGTGTGGCTGTCAGGAAAAG TCATGCTCCCAGCCCTGCAAGAGCAGCGGTTACCACCCTGTCATCCTCACAGATAGCTCTTTTCCTGCCTGGGGGAG CTTCCTGACATGGCGGTGCCCATCAGTACTCTGATGAATATGTGAGGGTGACCTGTTGCAGATCTCAGGATTCCTCT TTCTGAGATGTGTCTCTGTCCCTCCAGTTCTGTCCTGCAAACTCTGCCTGTCCTGGTTTCCGGGCTCACAGCTCCAT CCCCTCAGTTTGGGAGCTGGCCAGGCCCTTCCTGTGTGCCCCTCCGGGTGCCCGGAACTCTCAGGGCAGTCGCAGCC TTTTCTTCCTTGGTTTTCTGTCACTCATCTGTCTCTGTACTTTGCTCTCCAGTTTCTTGAAAAGTGTGTTCTATTTA ATTTATTTTTTCTTTTTGGTTCTTTTTGGTGAGAGGGTCAATCTTGTCTCTTACTTATTTTATCTTGCCTGGAAACA GAAGTGCTGGGGTGTTTTTTAAAAAATAATTGTAAGATACATAATTTAAAGCTGTGCTATTATAGTATTTACATGAA GTTTTAAAAAGAAATTTGCTTCCCCTAAACTTTCTGATCATCGATAGCACTTTTTGAGGCAGGGAGCCACTAGTGGT CACCTATGTCTGTTAGCAATAGACAGACATGTCTGTTACTTCCAAAGGACTCTCACAGTGTAAACTGAGGACCCTCT TACAAGCTGGTACGGGAAGACCAGGCCTGCATCTGTCAGAGATGAGAATAAGACAGATTCTCCTAATCAAGCTGCAA GGAGCTCCTGCTGTAATCACTCCAGAATATATATCAAGAGTGGATGTGTGTTCTCTATGGCAAAAAACAGGCACTGG AAAATTTACATATTTTTAGTTTAAAATATGCATGCTCTTGGCCAATATGTTTGAACTGAAAAAAGATAGAATGTGGT TTTCTGAGTTATCTTCTTTTTCTTTTTTCTCAAGTTAGTTAAGATTTATAAGGTGCTTACTCAGAAATTCATGGATG AAGTACAACGTTCACCTTCACAACACTTTGCTCTGGTTACAAATTTTTGTGGTGTAGAAACCCCCAAAGTGCCTCGT ATTTTCTCACCCGAGTCCAGCCGGAGATACTGACCTTGGTCTAGATCGTCACCTCTGAACCATTCCTGAAGAAGTGG GTAGACACCCCCTCAGGACTGCTGTAAGAGTGGAACACATTCACGTATAACCAGCACAGAGGTTAGTATTCAGTAAG CACTCAATAAATGTTGGCTCTGAAGAGATTCTTCTCATTTGCATCATTGAAAAGGGGTCTTTGCAATGATGCTTATA GCCCAGTTTGGGGTGAATCATACCTCTAAGCAACTGTCATTGAAAGAAGGCCTTAATGCTGATAGTCATTGTTTCAG TTTTACTTTTTATTTCTTTGTCTTGGCTCTCTGGCTAGGCCTTTAATACAGTTTTGAATAGAAGTCGTGATAATGGG CATTCTTGTCTTGTTACTAAATTTAGAGGAAAAGCTTTCAGCATTCCACTGTTGATTTCCTGTGAACTCTATAGATT GTTTTGTGGATACCCATTTTCCCCCAGAATGAAAGATCTTTTTGAAATTATATTATTCTTTCTTTAATGAGGCCATT TTCATATTTGCATTTATGAGAAAATGTTTAAAAAAGTATGTGAACATAAATTATATAATCTTTTGAGTCAATCAGAA TTATACAATTGAAAAATAGTCTACAATTAATGATATTCAAGTATCTTTAGATTTTAAACTCATTAAATGAACATTGA AAGTTCTGTTTTCTAATTATAGAGCCGAGTTTAAATTGACAAGAGCAGCCACACTCGAGTTGTCTAAGTCAGAGGCT TTATAAACCTGTTGGATTTTCCAAGGGTTGCTTCTCTTGAATAAAGGTGTTTTTGTTTCTCACATATTTTACATTTT CAATGACCTGTTTAGGCAGAAGCACCTTTTGTCACTAATTAGTGTGGTGTGACAAGTAACATAATGCGTATCTGAAA AGCTCCACTGGGTGTAAGTGGAGTTCACACCTCTTAATGAGTGCCTTCAAGAGTGTTTTTGTCATTCCCAGGGAGAA CTAGTGCCCCTTGAATAGAAAACTGCTGTAAATGGAAAAAGGATGTGTCTTAGGAGCCTGGTGTAAGTGCAGATGGG CCCACCCTGTCCTCTGACTTTAGGGCCTTCCTCAAAGCCAAGGCCCTCTTGTGCGATGGGGCTCTTCTCCTTCTTAG AATTCCTACATCCAGAGTAATGGAGACGACTTGGGATCATGCTTCAGGATATATGTAGGTGCTCAACACAGGCTGGT TTCTGTTTTTAATTTCTGGCAGAATGAACCACTTGAAAATGAATTTTCATTACGATTTTAGGAGGGCTGAGAGTAGA TGAGGTAAGAGATCTGCCTAGGGGGCAGGATCGAAGGAGGCTGTCACTCTCAGATTTGTGCAAGTGACTTGTGTTCC CCAAATTCACATGTTGGAGCCCTAGCCCTCAGTACGTCGGAGCGTGACTGTATTTGGAGATGGGGCCTTTAAGCAGG TACTTAAAGTTAAATTGGGGTTGTTAGAATAGGCCCTAATTCAGCCTGACTAGTGTCCTTATAAGAAGAGGAGATTA GGGCACAGATATACCATAGGCTTCTGTGCACAGAGATGACCATGTGAAGTGGCAGCAAGGGGTGGCCGTCCGCAAGC CAAGAAGAGAGGCGTCAGAGAAAATCAACTCTGCCAACACCTTGAACCTAGACTTGCAGCATCCAGAGCTGTGAGAA AATAAATGTCTGTTCTTTAAACCACCCGGTCTATGGTATTTTGTTATGGCAGCCTGAGCAAATTCATTCAGCAAGTG CCAGCCTTGCACTCGCGGGAGAGTGAGTGCCTCCTGCAGTGCTCAGTCTGGTCTCAGGCTGAAATTCTAGGTAGATG CACATTTTCCCAGTTGGCAGGTGCTGGCTCATGCTGAAGGGGAACATATGCTCAGGATTCCTGCTTTATGTACTTCA AAAGGAGACATGTGACATGTTTCACTTAACTTCATCTAAATTTCCTAGACACACCAATCTGTAACGGTTTAGAACTC AACCCGGTGGTTATACAGCTTTCCTGAAGTGGAGAGGTGGGCTACTTATCTTGTATGAGTCCCATGAGAACTAAAGA ACAATGGTTCAAAAAACAGATGGATGTCTCCCATCTTACATTCCCTCCAGGAAGAAGTAGTTGAAGAAATAATGGAT ATAAATTTCCTTGAATTAAGAACACATGAAAGACTTAAGAATAAGAAGTCCAAGAGAGGACCAAAGAGGCAAAAAAA AAAAAAAAAGAAAAAAAAAAGAAAAATAACTGACGTCTATCTAGAAGAGCACAGTGAAATTTAAGAATAGAGAGAAA AATCTAAAACATTAGCAAGAACAAACAACACATATAAAGAACCAGTGGTCATATTGGCTTCAGATTTGTCAACAGCA ATATTGAATGTAAGAAGAGAATGGAGGCATGTTTTCAGCGTATTAAAGGAAAATAACTTAGAACAGAGTTTTATATG CAGCTAAAACTTTAAAATATGAGGGCTTGGCAAAACTATTCTCACAAGGCCTCAAAAGTTACACCTTAACAATTATA ATAAATGTAAATGAACTAAACTCACCAGTAAGAAAAAGACTGAAAAATGGAATAAAATATAAAATCCAAATATACGC CACTCAAAGGTTATCCAAAGTATAAGGACTTTCAAACTTTGAGGGTAAATGGATGGAAAGAAGCATGTCAGGAAAAT ACCCAAAGAAAACTGGCATAGCTATAATGATATGTAAAATAGATTCTAAGACTAACAGCCTTGTTAGAGATAGAAAA GGTGAATACACAATAACAAAACAAGTAAAATAGATTAACAGAGTAAAACATAAATGACAAAAATAATTTGTATTCTT GAAAATATATAGCCATGGAAGTATACATTTTAAGTTAGAAAATGAACAGAATAAACCTTTTAAAAAGCAGAAGAAAA GATCATTGGAAATTAGTGGAAAAAATACAAAGATCTTAAAAAACCATGAATTGTTTGTTTAAGAGAAAAAAATAGAT TAAAGGTAAGATATCTGAAATGGCTATATTAGCATTAGATGATGTATATTCTACAAAAAAAGTTACTAGGGTTAAAG AGAGATGTTACATAGCGATAAAGTATTCAGTTCACCAAAAAGACCTAACAGTCTTAAATGGCTATGTACCTGACAAC AGAATTCCAAAATACATGAAGCAAAAACGGAAAGGAGAAATAGATAAGTCCACAATTATAAATGGAGATTTCACCAT TTCTTACTGATCAACAGATCCTAAAATCAGTAAGGATATAGACAATTTGAACAGCATTATCTACTAACTTGATGTAA TTAACATTTCTGGAATACACTTAACAAAGGCCAAGATGTGGAAAATCCAGAATATTTGAAAGTTCTTAGTAACCCAT CATTCAAAGAAGTCACAGGGAAACTTAGAAAATGTTTTGAATTAAATGAACATGAAAGCACAACCTATCAAAAGCTG TGGGTTGCGGCTAAAAGTGTGCTTTGAGGGAAATTTATAACATTAAGTTTTTATAATAGCAAAAGGTCTCAAAACAA TGATCTAAGCTGCTGCTTTAAGAAAACAGAAAAGTAAGAGCAAGTCAAACCCAAGACAAACAGAAGGAAGGAGATAA TAAAGATAAAAACAAAGATGAATAAACATGGAAAACAGAAAAACAGTGGAAAATATCAATGAGTCAAAGAACTGGTT CTTTGGAAAAATATTAATTAAACTGATAAATCGGTGGCTAGACTGACCAAAAAAAAAAAAAAAGAGAGTGAAGTCAT ATATTAACAATGTTATGATGAGAGGGGCCATTGCTACAAAACATACTGACACTGAAAAAAAAAGTGCATAGCAGGAC AGTTTCGCTACCCATATGTTTGACTGCTTAGATAAATGAATAACTTTTTTTTTTTTTTTTTGGAGACAGAGTCTAGC TCTTTCTCCCAGCCTGGAGTTCAGAGGCACGATCTTGGCTCACTGCATCCTTCTCCTCCCGGGTTCAAGTGATTCTT CTGCCTCAGCCTCCTGAGTAGCTGGGACTACAGGTGAGCACCACCATGCCCACCTAATTTTTGTATTTTTAGTAGAG ACAGGATTTTACCATATTGGCCAGGCTGGTCTCAAACTCCTGGCCTCATCATCCACCCACCTCGGCCTCCCAAAATG CTGAGATTATAGGCGTGAGCCACTGTGCCCAGCCTGAATAACTTTTTTGAAAGGCACAGACTACCAAGCTCATTCAA GAAGAAATAGTCTTAAATCTATCATAATAATTGAATTTATAATTTAAGCCTTTCCATAATGAAAATTTCAGACGTAG TTGTCTTCATTGGGGAATTCTACAAAACATTTATGGAAGAAATAATGTCTGTTCTAGAAAATTCTTCCAGAAAATAC ACGAGGAAATACTTTCCAACTCTTTTTATGATGGCAGCATTACCTTGATACCACAACCAAATAAAGGGATTAGAAGA AAAATACAGACCAATATTCCTAATGTACATAAAATGCAAAAGTCCTCAGGAATATTACCAACTTGAAACCAGGAAAT ATATAAAGGATGATGGAACATGACCAACTTGAGTTTATCTTATGAATGTAAGACTGTTCAGTATTTCACAATTAATC AGTTTACCATATCAATAGGAAAAATGAAGATAAACCTGTGTTAATCTCGAAGAAATTTCCTGATTTGATAAAAAGGA ATCTATCAAATACTTATTGCTGACATAATTCCTAATGATAAAAGATTGAATGTTTTCCTCCTAAAATCAGAAATAAG ACAAAGATGTCTGTCCACTCTTCCTACAGCTATTCAGTGGAAGGTCCTAGCCAGTCCAGTACATCAAGGGAAAAAAT GATTTTGGAAAAGTAAGAAATAAAACTTTATTTATAGAAGGCATGATTGTCTATACAGAAAATTCCCAAAAACCTAC AAAACTACGCAACATACAAAAACCAATTGCCTTTCTATATACTAGCAATGAATATTGGATACTAAAATTTTAAATAT AGTTCCATTTATAATAGATGAAAATGTGCAGTAGTTCATGTCCAATGATATTTGCAGGATATATATACTGAAAACTA CAAAACCTTAAATAAAATAACATTTAAAGAAATGGAGAGATATACTGTGTTCTTAGATTGGAAGACTCAATATTCTC CCCAAACTGATGGATTTAACATGATTCCAGTCAAAATCACAGCAGGTCTTTTCCTAGAAACTGATAAGCCATTCTGA AATCTATGTGGAAATTTACAGTTGTACAAGTTTTCCAAAATTATTTTTGACAACCCATCACTAGAGAATGTTAAAGT AACTGATCATATAATTCCTTCAAGAATGGTCATTAAATTCTGGATTTCACAGTTTCTTTTAGTTCGTATTTCACAAG ATAGAAGCTTTACATTTTTCAGATTAAGGTAGTTTTCACCTTTGTGCATGCTACATCTTGGGGAAAATACTGAGACA AAATGACTCATTGGAATTATTTTTGATTTTTTGATACTCGGCTGACAATTATAGTCTGTGTTATGACAGTTATAGCC TGTGTCATACGTACTCAGAATTAACTAACATAGTCTTCCTCCTAACTTCCCTGCATATTGGAACATTAGAATTCTTT TGGTTGGTTGAAGAAACATCCTAGAATTTTAAAATATTTTGTATCTAATGTAATGCTGAAAACATGAAATCCACATA ATGTATGCGGCATAGCTAATATTAGAAGCATGTTCAAGGAGCAGCCTCTGGGGTTAATAGAGTTTAATTTGCAAGAC AAATGAAAGCTTATTTTCTCCTAGGCTCTCTGCTCATAATTCTAGTAGGGCCACTTTTGAAGAATAGTACCTTCAAC AGACACAGAGAACCCTTGTTGGACAGCAACAGAACTTGCTCACAGTGAGACCCGACATCCCCTCCGCTCAGTGGGCA TTTCCTACAGTGCAGAGTTGTGCGTTGAAAAGTGCCTCTTCACTCCCTACATGAGGTTCCTCACACAACCCCGCACC TGGGCGGGGGGCGGGCGTAGGGGGCATAAATGGCTTATCCCTATGAACTTCATCATTATGAAATGTAAAAATGGAC AGTCCCATTGCCCCAGAAACAGTGGGGGAGCTTCTAAGTAAATCCAGGTTTATCTGCATTTGGGAATATTCTGCATT TTTTGAAAATGATGTTCCCAGGAATCTTTATAACCAAGGAGAATCCTTACTGTAAACATTCCACCTGAATTTTTCCA AAAAAAAAATAGATCATTAAGAACAATAGACAAAGGAATATGCTTTTAAAAATAGTGATTATCTCTGAAGGTGGGAT TGTGGCTAATTTTTAACTACCCTGGTGTGACCTTATGTGACCCTGTGTGAGTCAACATGTCTGGCCTCAGGGGTGTG TGTGGAGGGGGCACCCTTTGCCATTGCATTACACAAGTCTTTGAACTGACTGTCCTTCTTACTAAATGCTATTAGCA TCTCTTAGTCACTGTGGCAGCCAAAAATGCTTCCACGTTTTCTAAGCATTCCACAGGTGTATGTTCCTGGCCTGATT CAGGATAGACCAGTAACTCCTGAAAGTAATCAAAGGGGCCGGGTATTGTGGCTCACGTCTGTAGTCACAGCTACTTG GGAGGCTGAGGCGGGAAGACCACCTGAGCCCCGAAGTTCAAGACTGCAGTGAGCCGCGATCCCACCAAGGCATTCCA GCCTGGGCTGCAGAATAAGAACCTGTCTCTTAAGAAAAAAAAAAGAAAAAAAGGTAATAAAAGAGCACCAGGAAGCA TAGGTGATGCACCTCCCATGTAGAGAAGGCTGGGCGAGCGAATGTGTAGTGGTTTCTGCGGCTCCATTGTTAGTGAT GACTTACGGATGGAAAAGGGAGTTTTGTAGCTGAGTGAAAACTGTTAGAAAACCAAATGCCGCTGTCATGTTTTTGT CTCGTGCGCTTCCCCTTCACCTTCATCCTTGGGCCCATGCTGTGGCCAACTGAGGAGTACCCTGAGGTTAAGCGTCC CCCATGGCAGGTGAGGCTGTGGGGTGGGAGGAGGGCTGGGAGATAGCAGATTAGAGATCTACAAGAGAATAGAAAGT TTGATTCAAGGAGAAAAACTGGAGTAAGCAGTTTGGAGAAGTCAGATGTTTAAAACATGAAAAGAAATGTAACTTTG AGTTGCAGTGGTTTTGAGTTTCCTGATCTTGATGTGCCAGTAACTCCCTGGGGGCTGACAAATCCAGGGGGAAGGTT CAAAGGCAGAGAGCCTGTTTGAGCTGGGCCAGGGCACAGAGGCTTGCTCAGAAGACGTGGACGGCAGCAGAGCCTGG GCTTGAGAGTCGAGCTCCACCTTCTTCAGGCTCTTTCCAGCGGGGGGGGACAAGGACCCACACATGCTAAGCGAGCA GGGACCCTCAAGCCTGTGACTGTTCGTGCCAGTGAAAAGGGCACCAGTGCACTGATTTCTTCCGTTCACCCAGAAAC AACGATTTCTTCAATGTTCACAGTAGTCTGTTAACCACCTCTTCGTGGTGCATCTTTGAGCCTGTGTTTACAGCAGT CCTCTCTTTGGTCTTAACAACCCAACGTGAATTTTGTGGCATGCTACAAATTGAGGCATCAAAGCTATGCTTTTTAC ATTGACTTTTGAGAAATTCGACAGAGTTCTGAAACCCAAATCTTGACCTTTCATGGATATGATGGTAACCTGGCTTG AGGTTTTTTTTCATCTCTTTCCCAAACTACCTGTTCTTTCCATCGGCATTTTAAGTAGCTTGAGCACTTGGTTTCAC TCTTCAATACTTTGATGATGCGAATATCTGCAGATGCCTACTGTATGCCCAGCCCAGCCTCTCTACAGAGAGACCTT CCCAGCCAGCCCCGGTCTAACCCATCATGCTGTTGAGTGTTCTTCCTAGCAGACATCATTATATTACTCATGCATTC ATTTTAGGGGGTTATGGTCTATTCCATTGCTCCCCTCTCCCAGAGCAGTGCTTTAGGGGAGCCAGGTCTGTCTTACT CCCGGTACACCCAGCACCTGTAACTCTCTAGCAAATCCTAGATGCTTGGTGAGTGAGTGAGTACATTGAAGTACATT GAGTACAGTGAGGCCACACAATTTAAAAAGTGACAATGGCAGTACACTGCGGCATGTGCTGGAAAATGAAGTCCAGG GTGCTGGGTGGCCTCATGGCAGGGAGGCTCCCCTAGGCTGGCGTCGGGGCCAGGCAGCCTGAGGATGTGAAATTCAA GAGGACACAGCAAGAGGCTGGAAGGAAACTAGTTGGGGACAGGGGTGAGAGGACTCGATTGAAGGGGTCCCACTGCA AAGGGCCCCATGAGCCCATCCAGAGTGCTTGGGTTCCCCCCCTGAGGCCTGGTGAGAGCCCGTGGAAGGGCCGAGCC TGGGGAGCTCACACCGAGGCCTCAATTCCTGCTTCCCTTTCAGACACGCATTCATCCTTAACTCTTTATCAGAAGGG AAATAAAACCTCCCAGATAGCTTGGTTGTGAGGATTAAATTAGGAAAAAAAATAAAGCTATAGCTGTTATTGGCCTT GCCCGGTGCTCCTGGACCGTGGTTAGTGGATGGAGCCTTTCCTCATCCTCCAGCTTCTTGCCTGTGTTTGTAGGGCT CACGACAGCTTGTAATTATTTCATTTGTGTGGGTCTTACTTTTTGCACATCTCTTCACTGCGTGGCAGGTCCTGTGG AGGCAGAGCTTGGGTCTGTTTTGTCCACTTTGTTCCCAGTCCAGGCAGAAGGATGGGCAGGGAGTGGACGTCCAGAT AATGAACCTGAAGTGAGTGAATGAATGCACTAAGCTCAAGTCAGGGTTTTCTTTACATAGAAATTCTCCCACGCTCC TTTTTGCTTTTAGTCTTGAGGATCAACAGTGTTCATGTACTAAATCAAGCCTGAAAGCCCTTTTTTTTTTTTTTTTT TTGAGATGGAGTCTTGCTCTGTCACCCATGCTGGAGTGCAGTAGCATGATCTTGGCTCACTGCAACCTCCGCCTCCT GGGTTCAAGCGATTCTGCTGCCTCAGCCTCCTGAGCAGCTGGGTTTATAGGCGCCCACCACCATGCCTAGCTGATTT TTGTATTTTTTTGGTAGAGATGAGGTTTTACCATGTTAATCAGGCTGGTCTCGAACTCCTGACCTCAGGTGATCCAC CCACCTCAGCCTCCCAAAGTGCTGGGATTACAGGCATGAACCACTGTACCTGGCCAAAAGCCCACGTTTGGCAGGGT GGCTGGCAGCCTTGGGGCCTCATCCACGGTGAGGTTGTGTTTGGCCTCTCTTTGCAGGTTGATGGCTGGAAGGGGGA AGGGTTCTGTGGTCAGGCCTGTGCTTCATGGAGATGTCTGACCGTTGGGACTGTACCTTTCATCCTTTGGATGAGAT GCTCTGCCTTGAGGCAGAAGCATGGAAAGCCGGTGGATTTGTGCGTGTCGGTGGTTTTCCACTGGGTCTCCAATCCG GACTCAGCATTGTGGGACCACCAGTGGCTCCCCCTGCCACTTCTTGTGGACACGTTCTTTCTCCTTAGAGCATCTGT TCCAGGCCAGCGGCGGTGTTACTTTCCAGCCTGGGGATGACTTTATGGCGTGATGTCTAATCTCCAGTGGGCAAGTG GTGAAGGTACTCTAGCATCCATCCAGTGGCCAGTCGGCCGAGGGCCTGTCTGCTCATGGGGAAGTGTCCTCTGATGG CAGCCCAGTGTCTTGCTAACCAGCAGACCACTGCAGAAGGCAACGCTGGCCTCCTAGAGGTGTGGCTTGTTTGCATC TCTATGTTTGTGACCAAGTCGTTTCCTTCCTTGGTCTCAGAATCTGCTTGGCACTTACTAATAGCATTTGTTCAACA TGCATGATCCCGGTATGTGAAGTACAGATATGACATTTATAAGCCAGACACTGCGTTTGACTAATGTTGGCAAAGTC ACACTAAGAGCCTCCCAGTGTTCAGATGGCGGAAGGGAAGGCAGATAGGAGTCCAGTCCCCAGGACTCTGAGGCCCC ACCTTGTGCCTTGGTAGAAGTGAAGACAGATCGCCTGGGGAAGTTGGAAGAAGTAGCCGAGACATTTCTCCTGGCTC AGAGCAGAGGGGGATTTACGTTGGCCTATGAAGGAAGAGTAAGTATGTAGAACCTTCACTTGTAGAGAGCTGAGACT TTACTGTCACCCTTGTTTTAGGGAGCCTACATGGCACCTAAGACTCAAATTTACATTTTTAACTAACTGAAGCCAAA TAAAAGACCTTTTATCCCAACTGGGTACACTCTAAAGTCTCGACATGATTATGCCTGTGATCCTGCACAAATCGTCT CAAAGATCTTGGAAAATTGGTTAGTGTCGGGGCACGGTGGCAGTTAGTACTGGGGCATTGGAAGCACCAGAGCTTAC CTTAGCTAGACGTTTCCATCCCGTGGCAGCTTAACACGTGGCATCCTCACTGGTCTTACAGCATTATGGGGAATGCA AAATAAGGACTTGTCTCCTTCCAGAAGCCTGCTGCCCATCAGAAATATATATATATATATATATATATATATATATA TATTTTCAGGGAATGGTAGAGAACAGTGGAGAAGTAGATGCTTGTCAGGCGTCTTGTCTGCTGGGCCAAGGTGAATC AGGAGGTGAGAGCGGCGGCTTTCCTCTTGTTTGGGCAGCGGGTGAAGGACAGGCGTGGCAGGCTGTATGGTGAGTGT GGGCATGGCCGCCCCTCCGTCCTCTTTCCCCTCTGGCCCGTCTCGCATCTGAACACCTGGGAGTCTGTGATTAGACC TGAGAAGAGGAAATGAGGGGCAGTTCCCTTCCATTTTCCTCCCAGCCGCTCAGTCCAGGTGTGTGTCAGTTTGCACG CCTCCCTCTGGAAGTCCGTGTCTCTGCGAGGCTCCTCAGCCCCGGTAGCCCCCACGCCAAGTCCCATTTACTCAGGG TTTCTGCTGGTGACGGCTCTAATAGGCACTTCACCTGCTCTGCGGAGGCGCCTGCGGAATGGTGGTCGCTCTCATTA GCTCTGCTCACCCGCCCAGCGTTACAGGCAGCTGGGGAGGGATTCAGGGGCTGCTGTGTTCAGCAGTAATCAGAGCC CTTCTGTGCACATTCGACGCTCAAGTGTGTAGTTTACCACGGGAGATCAAGAAATACGTTAAATGCATAAAATTAAA GGCAGCGGCCTGCAGGATCCCCAGGTGGCGTGTGCTGGGAATTGGATGCACTTGGCTGTGTGTGTCACTTAGGAAAT GCGCTAGGGGGTCAGCAGCTTTCCCATCGAAGCGTTGGTTAGTTTGTGAAAAGTAAAATTCCAAATATTTGAAATAA TAAGGGGCCAGATACGTTTGGAGCTTGAGAAGAACTATTTCACCAGCCAAGAGATCCTAAACATTTTTTGGCTTAAT AGAAAATAATAGCCTATTTTGAACAATAATCTTTGTAAATCCAAGAAGAAAAAACAGTTCATCTCCCTTTATAGGGT CTGATTTGTGTCTGTTCAATTTAAATAGCGGCAACAGAAATACAAGTGTCTTTGGGTGATTTTGTTCTGCTCTTACC AACAGATGACAATCACGTCTTATGGTTTTTGATTTGACAGAACGGTTCATTTCATCAGCTTTCTAGCAGCCCATTTG ATTCAATTTCCCTCTGCAGTGTTTTACTGAAAATGACAAAAATGTTCTTTTACATAAAGCCTGCGAGTCTGTGAAGG AACATAAATGTGCTGTGCTGTTTACATACTAACTTAATTGTAATTTAAATGCCCTTTTCATTCCATAAATATAGCCA TAGGCTTTTCATTTCCTTCAAGCCTTCAGCTCACAGAGCAGTTTATATGTAAACTGGAGTCAGTTGCTTCATAAAGT GAATTTCTGGAGGAAATAATATAGTATATATCAAAATCTAGATAGGTAATGGGAAATTGATTGTGACAATACAATAT TAGAAACACATCTCCATAATATTTTATTTATTACACTTTCCACCTTTTGCTGTCAAGGGAAAATATTTCATCTTGCA CCGCGAACTCCAGTCTGTAGAGATTTTGACCTCAATATTCTCACACAAATGAATATAGTGTGTAAGGTAGCACAATT TTCCCTGTGTAAAATCATGCATAAAAAACAATTCATTTAAAGCATCTAAATGTTTCCAAATTACTGCAGGTAATTTT TACAGCTTAATTCCTCCGAGACACCATTTTAAATGAACCCATAATTCAATTTTCAGACTTTGCATTTCAAATTTTTA TGTTGAAAGTAATTTCCTTTCTTGCTTAAGTTGTGCTCAGAACCAGATTCATAAAATGTGTATGATGTATGTCTGTA GAATCAGTGACTGTTAGAAGCTTAATAACAAATATGCAAGGAAGAAATTAAATGATTTTTCTCTTCAGTAGTTCAGT TTAACATTGATTTCTAAAAGAAATGATTCTGACTTTGCCGCTAAGTTTCTTTCTTTCACTTTTTTTCTTTTCTTTTT TCTTCTTGCAGTGTTCCAGGGGATAAAACTGTCCTTGTATCTGTATTTTGAATGATTTGGAATCTTTTATCTTTGCA TATTCCTTTAGACCAGAAAAGAAGTTGAATAATGTTCAGACTGGCTCCCATGTTAAGGTAGCACTTAGAGATTCTCT GAATATCATCCCTGAAGCTATGGCTTCCTAAAGACTTTTGACCTCTTAAATTAGTGAAGAGTGGACACCACCGCTTA AGCCCTGCTAAGACCAGTTAAAAGCATCTGTGGTCTGGGCGGAGGGAGCTTTGAGTACAATGGAAAATGGCACCACC GTGTTCATGCTTGCGAGCTTTCCATCCTGATGGCTCTCCTCTGCCTTTTCCTGTGGCTGTGGGATTTTTCAAAACCC TTTTCCATGTCTGCCCTTCAATAGAACACTCCAAGTATCCAGTTTTATTAATGTCCAGAAGCGTAAATCACAGCCCC CTGTTTTGTCATTCCAGTCATTTTAGTCACTAAAGGATTACTTCAAGTGACTGAACTCGGCTGTTATCTGTTTGAGT TTTTGCAGCCAGGTCATCTCAGGGGACTGGGAACAAGCTTTCTGCCCCCTTAACTATGTCGAGTGGATGGCTTTAGC TGGACCACTTTTTGTGATTGATGTACATTCTGTGTCGCTGTATCTTCCCGCGACGTGGTAATGCAAACTTAGAAAAA AAAATAAGATGGAAATGGTGCTGCAAGTTTTTAAAATCAAGGGTTGGGCAGTGCAGGGGGAGCTATTGTTTAAAAAA TAAAAGTTGGGAGGAAGATATAATGGATTTTGTCAGTGGTATTAGGGCGATGAGGCATTCTTGAGTTCAGCAAATAT TGGCAGATAATTCTGCTTTATGGAAACTATTAAATGTAATTGGTCACATCAGAGACACAATTGGCATCTGTACTTTG AAGCTGAATCTCCTCAGGTTGAGAGAGAGAAAGAGAGATTAAGACGGACTCTTCAGAGACCATGGACTACAGGGCCC AAAGTTATAAATCTGGAAGATGTAAATAAAGGAATATTTTAGGCTAACGTTGCTCCAAGAAGCAGTATTTGCCTGTA AGAAATGTAATAATAGAGGTGCCAATACAAGATAGCAAAAGTTACCCAGGAAAGATGTTAAGTGACATGCTGTCTTC ATTTTTAAACAGGACAAATTATTTAATTGGGAATTTTGCCGACACTGTTGAAGCAATTATCATATTTAAACTGTTTA TCTTCTCCACATTCTGCCTGTATTTTTTTTCTTCTTTGCATTAATCTTTTTCCTCTTTAGCATCTAGAGAGGGTCCC TTTCTCGTGGAGATCGGCATTGTAGACCTTAAAAGGTCTCACTTGAGAAATATTTTTGATATTATTGAATAATTATT AATTCATGCAAGAAACAAAAGGAAGGGCACTCACCGGCTCGTGGCCAGAGCAGACTCTCATTCTTTACTTCTGAGTG TTTGGGGGCTTCTAGCGAATAATCCCTGGATGACGGGTCCTAGTGGTTTTGAGACCACCAGTGGGTAGCCTCGCCCT GGGAGGGGCTTGAGCAGCCTTGAGGTGCTTTTGTGCTTTGCACGTTCGTAATTGTGAACCTCAACGCAAAGCAGGTG TTGTTGTAAATCTGGTGTCTCTGTGGAAGCAATGCCAGTCCTCTAATTCATCTTCGCCAAGCTAGAGGACGCGAGCA CATGTGCAGGTCTGTGACCTAAAGCCCTTGCTGGCCAGGGTGCTATTCTTACCTTTGGGCTTCCAGAAAGAGCCAGC TCCTGGTGGAGTGAAGGTCCTACCTCCAGATGCCTCCGAAGGTGCCGGTACGTGATTGGAATAGGAATGCGCATGAA CGGAGAAAACCCTCACCATTGCGTCCTTTTGTTCTTTTTGCGAGGAAGAGATCCATGGGTTGGCTCTTACTGAACAT GAAATGTTGGAAGTGGAACACTGCAGTGTTACCAGTGAGCTGTGTAATCGCCACCTCTGACAGGGATCCTGCTCGAG GAGCACGTCTTTCCTTGGTGTGGCCGTCTCCAAGTACTTTGGGTGGTGGTAAATTTACCCTTCTCGGTCACACACTG TGAAGATGTGAGTTTCCCAATTTCAGCATTTGGAGCCATGTCCTTGGTGAGGGCAGGAGGAATTTGTCACCCTTTCT CGATGTGCCTGGGACTAGGGCAGAGACGGTTAGTCTCAGGAGAGCCAGGTGTGCCAGCTGCCCCTGCCTGCCATGGC TCCCGCATTGTGGCCAGGACTTTTTCTGGCAGTGGTACTGCCCGAGGGCCGCAGTCCTGGTCACATTCCACTTGGGG ACAGAGTGTGCAAGGGGCTGATCTGAAGATCCGGCTGGAACCTGGGAGCCACAGGATCCTGGGGACCCTCTTTGGCT GTACCCCGTACCTCTGTATTTGCTCCCACATGCTGTCTGATTTCTCTGGATGGCCTGGATGCCTCTGTTCCCAGCAT CTCCTCGGCATGCTCCCTGCAGCCCTTGGCCTGTGTCCAGTGGGCCCCGACCCGGAGGCTCCTGCCCCGTAGCTCAG GTCCCGTCTTGGCCTCAGCCTTCCCTTAGGTGCTCGTTTCTCCAGTACCTGCAGCACCGGCGCTTTTAGATTGCATT CTTTATGCATTAATTAGTAATGTGGTGGTCCCAGGTGACTAATAAGCTAAAGAAAGAACTAGTGTGAGTGAATTTCT TTAAAGGTCATTTGGCTCTAGGAAAACCTTTTTTTTAAGAAAAAAAAAAGCATTTTATAGAAATGTTCAATGGCAAA GTGAAATGTAAAGTATTACATGTTTTTTACTATAGCGAACAAATTGCCCCTTTTTATTCCGTTTATGTCTTCATCTG CTACATTTAGCCAGATATATTTGTCTACCTAATTTTCTGCAAACTGGAAGACATAATCCCCCAATGTGAAAACACCA AGAGTTACATGATGATAATTATAACTCATGAGGGGAAAAGTGTCGCCTATAGAATCCATACCGGTCGTTTGCCTGAG AGAACGTGCTCATCCTGGCCCTGTTGGATTGTCTCCACAGATGTTTTTCATTTCGTATTTGGTTTGATTAAAGACAA AATTCACCCATCAAAAGGAAGAACGGCCCTGCAGAGTGTGAGTTCTTCATTCGAGAGCATGTGCTAGTTAAAAAGAA AGAAAAAAGATATCTTAATCCAATTTCTGAGCTGCACTGATCCCAAATTGAACTGAATTTCCATTAATAACTCAACC TAAAAATCCTTTAGGTGTTCTCTGGGGGCTGGAAACAGAGGCCGGTGGCGTGGTGTGATGTTCAGCGTTGGCCAGGA TCACTTGCTTCACCCAGGTGTTGGGCCTGCACACTGAGTGCCCGAGAGGAAAGGTTCCGGGAAAGCTGCTGTCGGAA GCCGGGGTTTGTGGGATCGTGTGTAGGAGTAGATGTGGAGGGGCTTGTGTGAATGGGGGAGTTATGAAACCAGGACT GAAGACAACAGAAAGGCCCCCTGGGAGACAGGTCCCTGGCTTGCCCTGTGCTTCCCCGCCACTCGGGGGGCCGGCCA TGCGGAGAACACTCTCCTCTCCCAGCCAGGCTTCAGAACTGGGGGGCTCGGCCTCCTTCCTCTGGCCCCAGCAGCCC CCGAGCGGAGACGCTGGAGTTCCAGTAGAGTGGAGGGAAAAGTGTCTTTGACAGCGAGGTGAGCTGCCTGCCTGCAC GGTGGGTCCCCTGGGTGACTCAGGCCCCCTTCCTGGAAGGCAAAGGGAGGGGCACGTTTCTGACCCTCCTGAGGGAA CGGAGGAGACTGGAGAGGGAACATTCTAGTATGTGCGTGCGCCCATGCGAAGGCTTTCAAAGGCCTGAGAGCAGACT TCAGAGCTGATCACAAAGTTGAAGGTGGGAGGAGGGGAGCAAGAAAGCATCGGCACTCAGAGTCCCCAGGCATGGGC ACCTGACACGGTGCTGTCCCCCGCAGTGGCTGCTGCTGTTGGTGTGCCTACTGTGTGGGGGCTTCTGTGGGAGAGGA CCCTGTCCTCCGCACGTCAGCTGCCGGAGAGTCAGCTGGTGCCAGATGGCCGGCCGGCCTGCCTGCCTCTGGGAGGC CTCAGTGCCTGTTGGATTCCCTGCCATGCAGGGCGCACCCACGGGGTGACGTGTGGGACCCTCCCAAGCGTGCCCGT GCACCCATGCAGCTGTGGCCGCAGTCTCCACTGTTTTGAATATTTGTTGTGTTCCTCCTGGTAGGATGCATGATGTT TTGGGACTGTGCTGGCAGCTCTGTCTTCCAGTTCCTTCCCAAGCAGTGTGGGCTGAGGGTGCCAGCAGAATGCCCGC CCCAGGACACGATCCAGTGGCTCTCGGGATCCCTCCAGGGTTGGCTGAGAATATATCAGTGGCACTGATCTTGTCCC AGAATGCTTGGTCGTCCGGTCGGCTGTGACAAAAGAGAAACATGGTCCGGGTTATACACAGCCTTTCCTTGTCTTTA TTTCTAGACACCCTTTATTTCAAGACTCTACTCCACTGGACTTCATGTCCGGTTATTAATAAGCAGTTCTCGTTCTG TCATCGAGCCTGCTCTGGAGTTAACTGTCAAATAATCTGAACTAAAAAGTTAAATCATATAGGCTTTGAAAATTAGC ATCCCTAAGTCTCTGTCCTTTTGTATTTGAAAACATTGATCATTTTCCACCCCCTCAGGATGGCCAGTATCCTCTGG ATTTATTGCTAACAGTAACTGCTCAGTTTCACACCCATCCTGAAACCATTTGAGGTGATCCTGTAATTGCCATGTGT GTGTATTACTAATCAAGGCTGCTCCCTCTACAAAACACTGAGTGGCCCCCAGGCCGAGGTGCTGCAGAGATGGCCCG TGTGGGAGGGGTAACGCCTGCAAACCCGACCCTGATCACCAATGGAGTGGCCTCACGTGGCCTCCCTGATGGTGGCC CTTGACTGTGGTCAGCACTGGACTGGCCCACCCTATGAGATGTCTCAGGCAGCTTGCAAGATTACATCATGTGATGG CCAATGCTTCTGCTAAACAAGAGGCAGGGAGCCGAATGGACAGGCCTCAGGAAGCACAGCGTGGTGGCCGTCACAGC CTTCTGGGAAGGCTATTTCTGCATCACTGAGGCCCCACAGCCCAGGGTGTTTAGGATGAAGGGCACAGATTTCCAGA TGTGAAGAGTGTCAGGTGGGCTAGGGGCAGGAGAAGCATGAGGAGGACCCTGTGCTGGGGATGAGAGGAGAGCGGCC CTGCAGTCAAGGGGCCAGGTGGAGCCACCTTTGGGGATGCGGGGAGAACTTGGTGGCCCTCAGAGACCCCTGCTTGG AGACCCCACCTCCCCCTTGACAAGACTGCGGCCTGTGCTGGGCACAGCTGGCCCAGAGCGAGGGCCACACCCTACTA CTGCACCCTCCACGCCTGTCTGTCCTTGCCCACCTCGGTGGCTGCCTCGCCGACCCCCGCCTTGCAGCTGGAGTAGC TGTGCTGATAAAGATGCAGGTGGGAAGCTGGATGGTGAGGTAGCCCATCTGTGGTTTGGGATCCGAGAAGACGGCAA GACCTCAGCTTCATGAGAAGAGAGACCTGAAGATCCATGCTGGGGAAGGGCGAGGATGGAAAGGGGTGCAGGGCGTG GAGCAGGTGCCCAGGTCGGGTACAGAGACAGGCCTGGGACCTGGAGGACCTGCACGCGGGGTCGGGCACCTGCCACA CTCTCCCATGTCTTTGAAGCCTCTTGTGGTAGAAGAGAAGGTGCTCATATTTTACCTTCTGTGCCTTAGTATGTAGT TTGGTATTGTGTTAAGTGGACAGAAATCACATACCCCTGCTCACTCTGAGCCTGCATCCTTTGACCGTCACTGTGAA CCTCAGATCGAGACACCAGCCTCAGCGGGGGCTGAGATAGGGCATTCCCCATTCCCACTGGCTGCTTGGAGGGCTGT GAGGGACCTGCTGCCTGGAGCACAGGGCCTGTATGGGTGGGTAAGTCCCTGGAGAGACAGGAGGAGGCAGGGGTGTG CAAGCCTCTTGAGGTATGAGGAAGGTGGCCGGGGTCTCGGGAAGCTTGTCTGGCCCTGGCAGGCAGGACAAGGGCCC CCTCCTGCCCATACCTCTACCTTAGGCCCCTTGTCATGACACGTGATACCTGAGGGTGGGGGGCTGTATGGGCATTT GGAATGTGACGGCACAAGCCACAGTGGGTCACTTGTTTCTACAAATGCCTGAGAAGAAAACTATTCTTCTAACTCAG AGAAGTGGGATTTCCCACTGAAAATCAGTGGAAATCACTTATAATGGTCTTTGCAAATTCCTTGCATCTAAGGAAGT GCCTGTGGAGCAGCAGTGGGAGGAAACCATTCAGGCTCGATGGAGAGGGTGACCGCTGCACCCTCATCCCTGGTGTC AGCACACTACCGAGGTGCTCCTGGTTTAAGGACGTTTGTGCACCCACCCGGAGGTATGCATGGAAAGAAAGCACTGC AGCCATTCCCCAGCCAGTGTAGGGGTGGGACACAGTCCCGTGGGGGCCCCGCGGAGGGCCTGGGCAGATGCAGGGCT GCGCACTGCCGTGGTTTCCTGTGTGTCTGCCAGGAGTGAAGACCTGAGCAGCCTCACTGGCCTGGTGTGGAGGGGCC GCCTCCGAGCATGCGGAGAAGTGGTTTGCATGGCTGGAAGTCAATGTTTATCTGTCTTTAGACTCAGTTGAAGACAC ATTTCTTTCACACATTACCATTTTTAAAACCCATTAAGGTCTGATTTTCCAAGGCGCTGACCCTGACGCTTCTAGAG GGCCTCATCCCGGAAGCTGGTGGGGAGAGCAGGAAAATCTTCCCCGTTGAGTTTTAAACGTCCAGACCTTTGTTGTC TCCTTGTGACCCTTGCAGGAGAGCTCTCGTGGCTTCTGTGCATGTTCGTGTGTGACTCGCCCGGCTGAGGGACTGGC ATAAACAAACAGGGATGAGTTTGTAAAACACTTTGTAATCCCTTGAAAAGGAGAAAAATATAAATGAAAATCACTGT TTAACAGTGAAGGCAGGAAGGGAGCTGTTCCTCAGTATAGCTGTGGGTTGTCGTTACGTGAGCAGGAGGGAATTTCA CTTTGGATGGATTTGGATTTTCTCTTCATGCTCATGCTCTCTAAATCTTGCAAAAGCACATGGTAAACACACAGCGC ATGTGTTTCTGGAGGAAACGCACAGTGTCCATATAGAGCAGGCACTGATTTTGCACTGAAAGAACGTGGATGAGAAG TTCCACGTGTTTAAATAAACTGGCAACAGCGGTCCCAGATAGCTTAGCCGGGCAGCAGTCAGAGGTGCCCTGGCCTG GGGACTGAGCTAAGGTCTCAGAAGGGTCAGCTAGCCAAGCTCTTCACCGGACAACCCCTGAATTAACCAGACACATC ACAGATGCAGACTGTTGATTCCAGGGAACCCAGGTGAAAGGCCAAGGAGAGGTGAGGTAGTTGGTGCGGTGCCCCTC CTGGAGTCCCTTCAGGACTATGGCACTACAGTTCTATGTAAGCTGTCACCCAGGACTCATCTTCAGTGGAGTCTGGA CTGCAGTCGGGAGGTGACAAACATCCATGGCATGAGGGCAGCAAGAGCCCCTCTCTTGGGAACCAGTTGTGGCCACT ATCTGCCCAGTGAACATGGGCTTCAGCCCCGAGCATGGGAGGGCTCCTGGTTGGGCATCTCCCTGGGAGTTCCTTGG TACTGCCTCCTGCAGGGTGAGGAGGGGTGGGGCCTACTTCCAGGCCTCTGGAAGCAGCATCGGCATGTCCTTTCTGG TGTGAACTCCAAATATCTGAGATGGTCTCAGTTAATTTAGAAAGTTTATTTTGCCAAGATTAAGGACATGCGCCTGT GACCCAGCCTCAGGAGGTCCTGATGACACGTGCCCCAGGTGGTCGGGGCACCGCTTGGTTTTATAGATTTTAGAGAG ACATGATACATCAATTAATATATGTAAGAAGTACATTGGTTCTGTCCAGAAAGGCAGGGACAACTCGAAGCAGGGAA GGGGCTTCCAGGTCACAGGTAGGTGAGAGTCAAGTGGTTGCACCGAGTTTCTGATAAGCCTCTCCAAAGGAGGCAAT CAGAGATGTCTTTCTCTCTGTGAGCAGAGGGATGACTGAATACAACGGGAGGCAGGTTGGCCCTGAGCAGTTTCCAG CTTGACTTTCCCCTTTAGATTAGTAATTTGGGGGCCCCAAGGTTCTCCTTTCATGCTGGGAGGAGGGTTTTTCCACA CGTCTCAGACCCAACTCTCGGGCTTCTTAACCCAAAAGAACTTCCACGCAGAAGGCAGCGAAAATAGTGCACTTCAT TTTCTAAGACATCTAAAGCTGCCACAATGACTGTGAGTTAGGTGAGCTCTATGAATTAGGCTAGGGATTTGCAACCA CTTAGCAAGTTCTAAGTAACTGTAAAAGATCCCATAGACTGCAAATTGTGTTTCATTTGGGGACAGATACCCTGTCT TGGGGATTTAAGTGTGTTTACCTGCCCAGAGTTGACACAGACGACTGTCCCTTAGCTTCCTTTCCTCATGCTGGTTT TAAGTGCTTCGAATCGGGATTTTCCTACCCATCTGATGCATTCCATTGAAAATTCAGGAATACACATGTATGTGACT GTTTATTGGCAAGTAATTGTGAAATAGGCCAGAAATCTACCAGCTTCCTTTAATTAATGTAACAAATACTTAGCAAG GACCTCCTGCATTCTAGGGATTGTGGGGGTGGGGGGGGGAGCAACAAACAAAACAGATAAACCCTGCCCTGAGGCGC CCCTTGTCTTGTCTGGGGGTGGAGGTGGGGCAGGCGTGAGACAGAAGAAACAGGTAAAATACAGTTCAAAGTCAGGT GCTGGTCAGTGTCTTGGAGAATTCTAAAGCAGGGAGGAGGGATGGGGTGAGTTTTAAATGTGGTGACTGCTGAAGAA ATGGCCTCTGACAGCCTGGAGGAGGAGCTGGAGCTGGGGGGGGGACAGGCTGGCCAGCTCCTCCAACACACACACAC ACACACACACACACACACACGCACACACACACACACACACACCCCCTCTCCCCCAAGGACCCACAAGGACTGCGGCC TCTTAGCGTGTGTGGAGTCATCACACTGACCCTTAGCAGTGTGGCCTGACTTCATGAGGCCCCCGTTGTCATGGAGA TGCTGGGAATGGGAGGATTTGCCACCCTCCAGTGAGGAGCCCCCATCCCATGAGTGCAAAGCCTGAACCGGCCCTTC CCTAATTGGCACCTCTATCCTATTTTCTTTGGCTGAGGAAATTCTCGTAAATGTATATTTAATGTGGTGTTGAGATT GGTAGTTCTCCCCCATCCCCCAAAGATCATCAAATCTCTGGGGGTCTTTCAAAACCTAAAGGTGATCTCCCTGGTCC TATTATGCTGTTTTGTTTTTGCTTCTTTTCTTTTTCTTGAAAGACCTAATTGTGGAGTTTTTTCCCCCTTCTATGAA TCCCAAGTAAGGAACTAAAATGGAAGACGGGCTTCTGGCTCCCGCTGCAGCAGATGGGATAATTCTACAGGTGATTG CCCTGGAGGTCTGCAAGGGAGGGAGAGCGGCACCATTAGACTAACACAGCTCTATTATTGACTAATGACGAACAGCG TCGCCTTGCCTTGCCACCTTAAAACAGACACAGGGATTTGCTACTCTATTTGTTTTATAAAATATGCATTGAGAGCA ACACCAAAATGCTGAATGACAGGTGACCCTCAGAGCACGCCTGGTATGCACTGCGTTAGCTTGCCAGTATTTCTTAG AGACTTATTTGATTCTAATTCAGTAGGATTTTTAACCCCAACATTTTGAAATCTTGCTTTGAAAGTCCATGTTATTC CCATTAACGTCAGTGAGTTTTTCACATATCTAAATATCTAATATCATGCTTTGAGAATTGAAGCCCAGCCTGCTCTT TAAAAGCAAGGGAGCTCACCACATCCTGGAGTTCTGCAGTGATTTGACATGTCCTGGAAGAAATTATCTTCGCAAGA AAGTAAGTTTAGTTGATTGAAATGAAATTAACACCACATTTCTGTGTCATAATGTATTAACCTGATTGGCTAGCTGG ATTCCAGCCTGTTTACAAACATCCTGAAGCCTAGATGGCCCCTTCCTCAGTGTCCTTAACTGCTGTTCTCGCCCTTA GCTCGCGTGCCCAACCCTAAGGCTGAAAACGCCCCCTGTAGGCTACTGTAAGCTGTAGCATTCAAGGCCTCCCAGGT GTCGCTGTAATTTCTTGTATACATTTTAGCTCCATCTGTGGCATAGGTACCATTCAATGTTATCTTAATACACTAGC TTTGGATTTTGTGGCTGCAACAATAACAAAAAAAAATCCTTGGTTCTGATGACTTCATGTTGTTTCAGAAGATACTC AAGGCCGGGTGCAGTGGCTCACACCTGTAATCCCAACACTTTGGGAGGCCGAGGCAGGTGGATTACCTAAGGTCAGG AGTTCGAGACCAGCCTGGCCAACACAGTGAAACCCCATCTCTACTAAAAATACAAAACTTACCTGGGCATGGTGGTG CACACCCGTAGTCTCAGCTACTCGGGAGGCTGAGGCAGGAGAATTGCTTGAACCTGGGAGGCGGAGGTTGCAGTGAG CCAAGATCGCACCACTGCGCTCCAGCCTGGGTGGCAGAGCGAGACTCCCTGTGTGGAAAAAAAAAAAAAAGATACTC AGATGTAGCTGTCTCTGCCTGGGTTGTTTTCTAAATGCGTTTGGCTGGAGATAGAACAGATCCTGCCTCTATCCACG GCGTAAGGGAGTTAGCTTCATGGCAAGCTCTCTGGAGAACACAGGCCATTGTACCTGCTCATGAGAACAGAAGTGAA AGTCACACAGCATGCATATTAGTTCCGAGGCTACTGGCATTATTATTTAATGATGAGTTAGTGCAAATTGAGGGGTG AGGAAGTAGCATGTACAAAGGCCCTGAAGATGAATGACTGTGTGAAATTTTGAAGAGCAGTTTAGCCCGAAAATATT CCATGAGTAGCTGCCAGGAGAGCTTTTCTGGAACAGTATTAAACTTTAGAAAAAGTGTTCTGACATTTATAGACTGA CACGAAAACAGAAGTAAAAGTCTAAATTGAACCAAAGTCCTCCTTAAAGATTGCCTTTGAAATTGAGTAATGGCGGA TGCCCAGCACCTGATGTTTGGGCACGGGCGACAGGACGTAGGGTGGTCATCCGAATATCCCTGCCCCACTCCCCAGC TCTCTATGGCATTAGTAGATGCTGGAAGATGAGGCCGTGTTTTCTCTTTCATCACCCGTGCAGTGAAGGATTTGCGT GGTCTTTGGTGCTTTTCTTCTAAATAATGAACAATTATTTCTTGCGACATTTTGGTTTTTTACTGCCAATTCCAACA GCCATTTGTCTTTTCAGAGCTAAGTTTATAACTTCCTCCTTTCTCTCCTGTCACTTTTACCCCATTCTGATTTTTCA TCTCCCTGCCAATTGTGGTTTCGTTTCTGAGCACTCATTTATTAATCCTTCTTTTTCATTTCCCAGAACACGTGCTG GTGTAAATCTTTTTGCAGTTAAAGTATTACATTAACCTTTTTGATAGCATGGGGTGGTATCTCGGCGGATGGGCGAG TGCTGTTCGCCTGTGGTCTGTCTCAGGGGTTCTAGTTGGAAGGCGTCCTGCAGCCATTTCTTCAGAGGCTAAGGTGT GGGGGAGCACCTTGGGGAAGAAGAAAACATAGTTTCTGCCCTTACAAAGCTGATACCCCATCAAACTTGAGATTAAA ACAGACAAAAAACCCAAGAACCTACAGAGCACTGCAGAGAGGCCTTGGGACCCTCTGACAGAGTCAGTGGGGAGTCG GCTGGGAAGGGCAGCTTTTCTGTCCCAGCAGCCTTGCTTCCTCTTCAGCATGCTGAGGGACATGGAGCCCTCCACCA TTATCTGAATTGATTCCATCTTTCTCTCCTGCCTCTGCTGTGGGTTTCCATAATGTGAAATTAATGCATGTGCTTAG GTTGCGGGGGGCCAACCCCTCAGAAATATGCAAAGACTTCAAGGGAGACCTTTTCTTTTCACAAAGAGGAAGATCCC CCACCCCCAACACACACACACACACACACACACGCACACACATGCACAGTTTGCATAGCCGGAGTAGGGAGTATCTC TTTCTGGGGAATGTTCGTATGTCACTGTTGATTTTACATTGTTTTTCCTTTCTTTGACTTGAAAATTTTTAAAGTAA CACAAATTTATTATTTTAAAAACTTCAACTCTGGAAGTACAGCCGTAAAATCCTCTTTCCAGAAGCAATCAGTCTTA GTCCTTTGGAATGCAACCTTATAAATCTGTTTCTGTTGTAAATATACTTCCCTGTTCTTACCTGCCTTTACCTGGAG AGTACATAGTATATTCTTTTGTGAGTAGGACTTTAACTTCCTAATATATCATAGGCTTTCCGCAGCTGGTTCTGTAT TATGGATTACTGTAATTTATTAACTTTTCCCACTTTGAGTGGAAATTTAGTTTATTTCCAACTTTTTTTTTTTGCTT TTATGGTGTTATGAACATTTGTGTATGTCTGTATTTCTGTGCTGTGTGCAAGTATTTCTGTGGGATAGACGTCTAGG AGAGGAATTGTTTGGCCGAAGGGCATACACATTTAAATGTTCACGGATGCAGCCCAGACCCCCCTCATAAGGCTGTA CCAACTCATACTCTCAGCAGTAATAAAGACGCGTCCATTTCTCACACCTCTGCAGAGGATTTCTAAAATGGCTTTTT ATTTTTCAAAAAGCTTACATTTGACAACTATTGATGTTGTGCAACTTTCTGTGTTTATTAGCCAATTATATTTTTTT CTCCTTTGATTTACTTATACATATTCTTTGCTTTTTTCGATTGTTTCTTTTAGTAACTCTGCAGTATGGCTACTAGT CCTTTGTCTAACAAGTTATACATATTTTATTCTAGTCTCTTTTCTTAGCAGTTTAGTGGTGTCTTTTAGTCACGCTG GAGTTTTAAAGTTTTCCGCAATTTAATCTGACGATCAATTTACTGGAGTTTCCAGTTTTGAGTCCTGCTTTGAAGCG CCACTGTAAGATTTCGAAAAAGGACTATCTTGACTTACGGTTTTGTTTTAATATTTTTAATTTACTGGTAATTTGCT TTCGTGTGACCTGAGAATGATCTGACATAAGGTTTTTCTACATAGATGGTTTTGCTACACCCTTTCCATAGGCTTCA CTGTCATCTACCCTCATCACTGAAATGTCCCCTTCATCTTATTCTGACCTTTCCTGTGTGTGGGTCTAGATACAGGC TCTGTTCTGTCCATTGCTATTCCTGGGCCAGCTCTTCCCTGATAGTAATGTGGCTCTGCAGACAGTTCCACGTGTAG AGGATGTGTCCTTTTTCATTCTTGTTTGTAAATAAGAATGGGGGTGGGGACACGGGAGGCAGAATCATAAACAAATA GCCTGACAGAACAGGGATAATAAAAATTTAAAGAAAAAGGGGGTTAGGGGAAGTAGAGTAAGATCATGCCCTACTGT ATAGGTCATAGGTAGGAATCAAAGGAAACTATTTCAAGCTCACAGACCAGGAAGTGGAAGATTAGGCAAGGAAAAAA AGGAATTAAAATAGGGTGACCAGTCATCCCAGCTCGCCTGTGACTGAGGGGCCTCCCAGGACACGGAACTTTCCGTT TTAACACTGGGAAAATCCTGGGTAAACTGCTGGGACAAGTTGGTCACCCTAGATTACAGACGCTATAAAAGATATAC ACACAAAGGTACCACTAGAATAAAAACACTTTACAAAATATTAACCTAAAAGAAAAAGCAAAGAAGACTAAAGAGAC ACAGTAACCCTCACTGCAATTCCAGCATCAGCCAACAGGACAAAGCGGAAGCTGTGTCAGGAACTGTCAGGGGGCTT AGCTGCTTCTGGAAATGAAATTTCACGTTGGCTTGGTAAGGCAGCCCGTAGCTCAGCACATGTGAGCAGCACATGTG AAACACAGTGATTCACAAGGGAAGGGCAAAGGGATGGGCGGTGATTAATCAGGCAAATGAAACCATAGGGAAGCAGG GGTTGCCATCTTGGGATTAGACCAAGCAGAAATCAGGCTAAAAAGGCATTATAAGAGACAGATGAGAACACCTTAAA ATGCTAAAAGTCACACTCATAATGAAGATAAAACCCATGTAACACAGAAACCACCTGTATAAAGCAGAACCTACAGC ATACGCAAAGAGAAATAAATAGCAACACGGAAATTGTCAGAGATGTTAACACACTATTCTCAGTACAAGACAGGTCA AGTGGCCACAAGTAAGGAATGATACAGGAGACCCAAACAACCTATCCCGTCACAGATACGTATTGAAATGCATTCGC TGAGTAGAAAATACGTCTGTTTCTCAACATACATGGAATATTTACAAAAGCTTATCAAAAATTAGGTTATGAAGAAA AGTTTCATAAAAGAATTGTTACAATCAACATTCTCTGATCACAGTGCAATAAAACTATAAATTGTTAACAAAAACAA GGCCCATCCACCTAGAGACTTAAAACCTATTCTTGAATGAAAGTGTAAATATAAACCAACGTAATAGAATGTCTGTA AAATAATAACTCTGTGTATCAAAGGCATAGTTAATGCAGTGATTAGAGAATTCATGACTGTAGACACCTATATCAGT AAAAATGAGGGAAGAAAAATAAATGGAATTAATACCAAACTCAGAAAACTATGAAAAATTAAAAGGGAAGCCAAAAA AGTATAGGAAAGGAAATAATAGAAGCAGAAAATAACGAAGTAGAGAAGGAAAAAGCATTTATTTAATCAGTAAATCA AATTCTAGGTCCTTTGAAAAAAATTTAAACAGACAAATCATTAGTTATTTTATTCAAGGAAAAAAATGGGGAAACAC AAATATATAAAATGAGAATGACAACAGGGAAATAAGTATTGATACAGAAGTTTTTAAAAAGCACAAGAGACTAATTT GCAAATAAATATGAAAACATTTTTGAAGTGCATAATTAGGAAAAATCCAGTTTTAGCAAAATTAACCCCAGTAGAGA TAGAAAGCTTAAATAAACCAATTTACATGGAAGAAACAGAGAACATTTTCAAAGAACTACCTCACAAAATGGGTCTG AGCCCAGTTGCTTTCACAGGGGAATTCTAGTAAATCTTTTGTGTAGATGATTCTAATGATACATAAATTGTTTAAAA GCATAAGAAATGAAGGGAAACGTCCCATATTTTTTATGCAGCAAGTACAAATTGAAACCTAAACCTGGTAAAGATAG TATATTGTAAGGAAAATAAAAACTAATATAATATGGAAATATTGATGCAGAAATCCCAATGAATATTAGTGAATAGA TTCCGACACCTCATTAGTAGTATAATATACCTCAACAAATATAACCTTTCTATGCCCAGGAATGCAAGGGTAGTTTG GTATTAGGAAATCTGTTCATTGCATTGCAGATGAATAATATAATCATGCAAAAAGGAAGGAAAAAAGTACCAATGCA AGTAACTTAAACACTGTACTCTGATGACATGTACTCAATTGTAGGGGGAAAACTACAAATATTCTTCTTAATAACTT TGTTTTTCATAGTGATGTGGAATTATTTCATGTGTATGTGGATTGAGAAAATGACTAAGTGTGTTGATGTTTGGGAG AACCCCCTGTGAAGAAGAGGGACACTTACATGGAGTAGGCATCAAGGAAGAAGTTGGTGCAGTTGACTTGGAATCAG CATCATCTATATGGACTCATTATTTCTCAAACATGCACATATGTACACACTTCTTCCAGCTCTAGCTAGAACTAAAA AGGCCTAGAGGCAGTAATACCCCAGTCACAAAGAACACACCTAGTTCCCAGTCTTATTTTCAAAATACCATGCACCA CTGAAAAGGATCTTGTGCAGCTTGGAGAAATGGCTGATTCCAGGATGGGGGCAGCAAAGACACAAAAGGAGCCTAGA ACAAGAAGAATGATAAGCAGGATGAGAAGGAAGAATACAAAATGAGCCCAGAATTAGCAGGGGGAAGAGAAGCCAAT TTTTAAAATGGGCAAAACATTGGAACAGGCACTTCACCAAAGAATGCATATAAATGGAAGATAAGCATATGAAAATA TGCTCAACATCTTCACCCTTAGCAAATGAAAACTAAAACCACAAGGAGATTCCACTACATGCTTATTAGAATGGCTA AAGTGAACTGACAGTGCCAAGTGCTGATGAAGATGCAGAGCAGCCAGAGTCCATTGCGGGGGAAAATGCTGAATTAC AGCCACCAGGGTGACAGTCAGGCAGGTTTAAAAAATAGAGTTGAACATACACTTACCATATGGCACAGAAATCTGAC TTAAATGTTTACTCAAGTGAACAAAAACAGATACCTACTCACCCACCCATCCACTCCTTCAGTCACCCACCCACTCC ATCACTCTCTCTCTCTTCACCCACTCACCCACCCCCCCACCTACCCACTCACCCACCTACTCACTCCCCCACCCACC CCCCAACTCACTTACCCCCACACCCGCTCACCCACCCATCCTCTCCCCTCTCCCAACTCTCTCTCTCCCAACTCTCT CTCTCTCTCTAACTCAGAAAAGTGGCTTTACTCAGAGTCTCCCCAAGCCTTAAACAGCCATTCCCTCACTTGGAGAA CTGAGTAAGTAGATAAGCTGTAGTTCATCCCTACCATGCAGTTCAGATCAGCCCTGAGAAAGGAGCAGACAATTGAT ACATACAACATCATGAATGACTCTCAAGGCATTATGCATTTGATAGTGGAAGAAGCCAGACACTGAGATGACATTAC TCCACTTACAGGACTGACTGTGGGAGGCAAAACCACTGGGACAGCAAATCAGTGGTAGCGAGGGATGGGATTGAGGG GGAAAGAGGTTTCCTATAAAGGACCACAGGAAGTGCTTAGGATGATGGAACTCTGGCTTGATCTTAGTGATGGTCAT GTGACTGTAGGTGTTTGTCAAAACTTACAGAACTAAACACCAACAAGGGTGAGTTTTACTTCATGTAAAAAATGTTA ATGTCATGAAAGACAAAGGCTGTAAAACTGTTCCAGCTTGAGGGAGACTGAAGAGACATGGCGACTGAATACAACGT GTGCCTGTACTCCAGGGAAAGTGTATTGTGGGAGAGACTTAATTGGGACAATTGATGAAATTGGCTATGGATTATAT GTTAGATAAAAGCACTGTGTTGATGTAGAATTTCCTCAGTTTGACAACTGTACTGTATTATGGTTACCTAAGAGAAT ATTCTTAGGAAATAAGATGTGTGATATACCCAAAGTAACTCTCAAATGGTTCACACAAACAAATAATAATGTTTGTG TGGGGAGGGAGGGGGAGAGAAAGAAAAAACAGTGGCAAGATGGTAAAAACAGGTGAATCTAGGTAAAGAATATACTG GTGTTCTCTGTATTCTTGTAACTTTTCTGCAAGTTTAACGTTATTTGAAACAAATTTTAACTATTTCAGTGTTCTAG ATCATATATCTTTTTCCATGCTTTTAGAATGCATTTGTCAACTTTCACGTTTTAAAAATCCAGTTACAGTTTTGATT CAGGGTGCATTCACTATGTAGATTAATTTGGGATCACAATGAATTTCTGTTAATTTTGAGAAAATTTATATTTCTGA TATTTTCCTATCTAGGAATATGCTGTTTTTTCACTTATTCAGCACTTCTTTTATGTCTTTCCATAAAATTTTATACT ATGAGTCATAGAACTCATATTTTTTGCTAAGTTTAGCCATATATATTTTTTAATGTTTGTTTTATAAATGAGCTCTT TTCACCTCATTGAAATTACTAATTTATTATTTCTGACATACAGAAAAGCTGTTGACTTTTGTACTTGGCCATTCTGC TAAATTCTCTTATTAGTTCTAACATCAATTTATTCTCTTTAACTTTCTCTAATAATACCTGGAAATAATGATAATTT TGTTTATTCCTTCCTAATATTTTTATCTGTTATTTCTTTGTCTTGTCATGCATTGACCAGAACCTAGACAACAATGC TGAGAGTAGTGATACTGTTGATGGGGTGTGCCTACCCAACAGTTGTAGAAGTTTTTGTTTCTAATGTTTGCTGCTTA TGAAACATAAGATGGTAATTAACAGGTGATTGCTGGAAGTTCTTGGCTTGGTTCCCACACCTGAAAAGCATTTGTGA TTTACTGAAAACCAGTGCATGTTGAAGGCTTCCCAGCCTTACATAATTGAGATTCACTTTGACTGTCATATTGCCAA AAATGGACTCAAACCCTGAAGTTGTATCCATACCATCTTGAGAATCAGTTGTTTGAAGCGTCACCCATTGTTTCTAA CCATTTTTGTTATAAAGATAATACACACAGAGAGAAGCGCCTGCAGTGAATCTTTGTGTAACCATCATGCAGATCAG GAGACAAACCATGGCGGCCCCTCAGAACTCTGCCACGTGTTTCTTCCCATCTTTTCCTGCTGCTGCTCCCAGAAGTA ACCCTATTCCGACTTTTCTGGGTTTTCTTGGTAATCAATTTCTGGTTTTTCTTTATAGAGTTGTTGCCTAAGTATGC ACCCTTCAACACGATACTGTTGTTTTGCTTGTACTTTTTGTTAACTGTACATATAATAAATTATGTTATATATTTCT TTATTGCAGGCTCCTTTAATCCAGTGTTATGTTTGGGAGACGCACCCATACAACTGCATGTCACCGTAGCTTGTTCA TTTCCAGTGTCGTGTAGTGTCCCTTCATGTGAATTTATCACAGTTTATTAGTATCTTCCACTCTGATGGACATTTGG TAGTTGGAATTTTCATTACTAATGATGCCCCTGCCTGTGGTCATTCCTGCCTGCCTCTTGATGCACACAGGCACGCA TTTCTGCTGGGTATGTCCTAGTTCCAGACTCACTGGTTATACGGTATGGATGTAATTATTGTTAGTTTTCCAGTGTG ATTAAACATCAGATTAGCGCCCATTAGCAATGTCTGAGGCTGCCTGTTGCTCCACTCGGGTGCCCGTTTGGTATTAG GAGCTTCTTCAGCCATTCTTGTGTTTGTGTTCATGTATCTCATTGTGGTTTTAATTTTCATTTCTATAATGACTAAT GTGGTTGAATATGTTTTTGTATGATTATTTGCCATTTGTATTTGCTCTTTTTAAAGTATTTGTACAGGACTTTCACC CATTCTCTTATTGTGTTGCCTTATATAGGAATTATTTCTATGTTCTAGATATGGATCCTTTGCTGGCCATATATGTT GAAAGTATCTCCACTCTTTGGCTTGCCTTTCATTATTTTAATTGTGTCTTGCTGAACAGAAGGTTTTAATGTCAAGG TAGTTTTTAGTCAATCCCCTCATTTATGATTGGTGCATTTTGTCAGGTTTAAGAAGTCTTTTCTCTCCCATAAGAAC GTGAAGAAAACATATTCTCCTATGTTTTATAGACCATAGGTTGGCAAACTTTTTTCTGTAAAGGACTAAATAGTAAA CATTTTAAGCTTTGTGGACCACAGATAGTCTCTGCTACATATTCATCTTAAAAAAAAAAAAAGAATAACCTTTAGAA GTATAAAAACCATTCTTAGATGCTGGGCCAGAGTGGGCCAGGCTGTACTTTGCCTGTTCCTGTTCTAGAAGTCACAG TTATTTGCTCTCACCTTTACAGTTATGATTTCAGTTGCAGTTGGTTTTGTTTTTTGTTGTTTGGGTTTACAGCATGA GACATGGGACATCTCTAAATGGATATTTATTCATGTTGAGAAGTCCATTTATTCCCCATGTCTATGCAGTGACCCCT TTGTCACAAGTGAAATGAGTCTGTTTCTCAGTTGTCTATTCCATTTTATTGTCCTATTTGCTTATTCTCCCACCATA CCGCACTACTTTAATTATTGTAGCTTTATAACAAATTTTCAAATCTAATAGAGAAAGTACTTCATTTGTTCTTTTTA CAACTGTCTTGGTTATTCTTGGTTAAGACATTTCCATTAGTATATAAATGAATCAGTTTGTCACATTTCAGTTTCAG AGTAATTTTTAAAAGAACTTTTTCTTTTTGAAATGTAGAGAAGGTTTTTAGAAAATCTTTTTCTTCTTGAAATTGTA GAGAAACATCTCTACAATTTTGAGTCATCAAATCTGTAAACATATTATAGTCCTCCATTTGTTGAGGTTGTCTTAAT CTTTCCCAATCATGACTTAAAGCCTTTCCCTACAGAAGTCTTGCATACATTTTGTTGGATTTATTATTAGGTAGATG AATATTTAAATATATATAATCTATTGTAAGTGTTATGCATTTATTAATTGGCAGTGCTTCCTTAAGTGTTTGTTGGA GCATGCTGCTGAAAGCATCTTGGCTTATACATTTCCATAGAAGATTTAAAATTATAGGACACTAGGTTTCCTGATAT TTTTGTTTTTTTAAAAATCAACTTATTTTGTTTTACTGCAATTTTTTTTTGTTAAGAGACAGAGTCTCACTATGTTG CCCAGACTGGCTTCAAATTCCTGGACTCAAACAATTCTCCCACCTCAGCCTCCTAAAAAGCTGGAACTATGGATGCA CACCACTGTGCCCACCTCTAATTTCCTTTTTTTCCTGATCTTTCTGGCTGGTTGCTTACCAATTTTACTCACCTTTT TAATGACCCCACTTCTGGTTTTACTGCTTTTTCTTATTTTTTTGTTTGTTTTCTGTTTCATCAGTTTCCTATATGAG ATCAACCCACAGACATACAATTTTATTTGAGCATTTGTATTGCTTAGAAAGCATTTATAAAATTTTATTAAATTCTT CTCGACATTTTTCTTTCAGCATGGCATTACACTGCAGATTAATAATTTCCAATTAAAGGTTGGGTGGAAGCTCTTCA ATTGCACAGTTAAAAAGACTTTGAAATATGGAGATTTCTTTTTGTACTTGCCTTGGGATCCACAAAAAACAAAACAC TACCAGAACAGTAATTTGTGCTCAGAAATTATATCCACTGTAAATTTCTGTGGGAATAGAAATCTGGAGAAGTAAAT GAACAGCTGATGTTTCTTATGATTCAGTTGTTACTTTTCATCACAATAACTTTACTGAATTTTAAGCATATAATTAC TGTTTTGCCTTGTAACTTTAGGTTGAATTTATTTTTAAAAAATATAAAGTCCGATTTACAAAGCTATTTAATGTTTG ATAACAATAGTTGAAGGAAATTCTTTTGATTCAGAAAAATTTTAATATCAAACCTGAACCATAAAAATTGCAGTAAA ACGTTACTACCTCTGCTAAGTCATTGAAGTGCTTTGTGATACAGTATGTCAGGATATCCAGCTTCAGTTTCTGACTA AATTTCATAGAAATGATTGTCATTAGGTCCGTGAGCATCAATGAAGTACACCATTGATTACACTCTTTAAATAATGT GCAATAGATTCAGATTCACATATTTTATGCAGAGTACCTGCTAGTAGGTAATATAGTAACCATAGGCCTTAACATTT TCATAAGTTTTTCAGTTTAGCCAACTAATCCTTTTTTGCTTCACACAATTTCACAACCACCACTTATAACACATCTT AGCAGATTCCACTTCAGGTTGTACCGAAGTAGTACTTTCTCATTTTTCTGAAAATCTTTTCTTTGGATTTAAAAATT TTTTCTTTGAAAATATTCTTGCCATGGCCATAGTTTGCCAGCTGTTAATCTAGCTTTCAAAATAAGAAACCACAAAG AAGAGAAAATTGAAACATAGACCTAGTAGAAGAAAGGCAATAATGAAAAGCATAAATTAATGTAATGGAAATAAAAA AAATTTTTGAGAAAATCAAAGAAACCAATAGCTTATTTCAGTTTTGAAGACATCAATAAAAATGATAAAACTCAAAC TGAGAAAGAGATTACAAATTACCCAAATTGATGTGGTAAGAGGAAATAAGTACAGATCCTACAGACATTAAAAGATT ATGAAGGACATATTATAAACAGTTATGCCAGTAAATGTGACAACAGACAAAATGGACAGAATCATTGAAAGACACAA ACTATCAAAGCTCTCTCAGGAATTCGTAACTTAGTTGCTGTGGTGCCTGTATGACTGTACATGTCAGTACTCATTGA ACTGTATGTATGAAAAGATTTAATTTTTCTGTATGTGAAAATTATCTCAATTTACTTGACTTTAAAATATTGTTTAA TGCTGTCAGTCATTTGAGCTACACGTAGATTTACCATTTTTATTCTTTTCATCCTTTCTGTGTCTGTAGCCTTCTTT ATGTGATAATCCTCCTTCTGCCTGAAGAATACCTTTTCAGCAGCAGCAAGCTGAAGCCAGCCAGCTCCGAGAGCCAG TTCTTTAATTTACGAGAATTTTATGAGCTGGCTGTCAAACACACCCATTAGTAAAAATCGAATTACATAAATTTACA GTTAAATACATTATGTTTGGAAAGAGAACACACATTCAAAACTGATTCCTTCCAATTATTGTACTAATTGTGCTGTT ATCCATACCTTGGAGGCCATTTTTATGATGCTGTGATACTCCACATTTAGTGCTGTCACCTTGGTAGCTTGAAGTCA GTCCTGGTAGGAATATTTACACAGCAGAAATTAGCAAATGCTACTAATTAGAAGTTTATTTGATTATCTAGACCAGA GGTTGGTAAACTATGGCCTGGGGCTGGGGCTGGACATCTTTTTTTTTTTGGTAAATAAGTATTTGTCAACACAGCCA CACTCATTTGTTTATGTCTTGTCTGTGGCTGCTTTCATAGCTCAGTAGCAGTCAAGTGGGAGCAACTGAGACTTCCG GCCCACAAAACACAATATATTTACTATCTGGCCCTTTGAGAAAATGTTTGCCAACCAAAATCTAGATTTCAAAATGT GATGGAGAAGATGTTAGTAAGGTGCATTCATTTTCAGATGTCTTGTGTGTAACTGTTACATTTTGAATAGCTCCAGA AATTTAGCCCCTCTTCCAGTGGTTAAAAGTATCATCTCATTCAACAAAGAAATCGCTCACGTCATTGCCGAACACGT GAAGTTCTGACATAACATCTTCATCACCTCTGTCTCGTCACTTTGCAGCATTCATGTAGAACTGTGTGTTCACCAGT TGCAACCTCAGATTGGCTATGGACATGAGTTTGGCAAATATCAACAAAGTCATGCTATAAGAATCAGCTGGCTATAC AGAATTTACAACAAAGAGTATTGTGTATTGAATTATTTGTAAATTGTATACTTCACATCCTTTATATCAGTAGAGTT TATTCTTTAAGAAATGCACATCTACATGAATACTTGTTTTGGAATGCCAGCTAAACATTTATCAGTACACCATTGCC TTTATTTCATTTCACAGGGAATTGCTGGCAAGTTCTGGTTTCAGTTTCTTTTTTAGAAAATGTCTTAATTGTATCTG TATTTTTATATCATTTGTTATAGGGGTCTAGGTTTCCAGTTATTTTATTCAGAATCCAGAGATACTGTGACTTTTTC TTCAGGCTTCCATCATTCTTCTTGAGAAGTCAGCTCTTCTTTCAAGTAATGTCTTTTCTCTAAATTGAGATCTGACT AGGTGTGGACACCTTTTATTTATCTTGCTTTGAGATCACAGGGATTCCTGAATCTATAGCTCAATGTCTTTCAACAG TTTCAGAATGTTCTCATCTCTTACTTCTTCAGAGGTTATTTTTGCTCCATTCCCTTGAATTTCCTTCTTTGAACCCA TTTACGCATCGGTACACTTCCCTGTATCTTCTAAATCTCTTGTTCTCCGTTATTTCCCATTATTTTATATCCGTGGG GTCCATTGTGTTGATTTCTTCTGATCGCTCTAACAGTTCATTAGTTTTCTTTGCTACTGTGCCTAATTTGGTTTTAA ACCCATTCATAGAATACATTATTTCAATGATTTCTCAATTCTAGTATTTTCATTTTTTTAAATAGTTGGTGATTCTT TCCTGAATTTGAGATCTTAGCTTTTTTCCTGCTTGAAGGTAGTAAAGCATAGTTATTTTACATTCCAACACCTGGAA TCCCTGTGATCTATTTTTTGTCTTTATTTCTGCCATTTTTGTTCATATTGTCTTATCTCCTCATGGGTCTAGTTGTT TTTTATTTTGTGCCATATTAGTACATTGGAGAAACAGTGTGTAGAAATGACGTATTGCCCGGGAGGATGTTTTCTTC CTCTAGCAGTGCTTGGGGCACCGGCAGTGTGGGATCAGCTCATTCTAACCCTAGGGATTAATACGATCAGAGCAGGG CTGTGGCTCTTTTGAGGTTTGCTCTAGTGACAGCTCACTTTTACTCCTACAAGGTGGCCATTCAGATTCTTCCCCCA AGTTGAGGGTGCTCAAAGGACCCTGCAAGACACTGTCTGTTGCCCCAGGGCAGTCACCTCGCAGGAATGGTGCTTCA TCCCAGGGTAGGGGCAGCTCCTGTGCTGCTGTTTCCTCCTGTACGCTGGCCCAAAATTGTCGCTGTCTGCTCTTAGA ATATGACCCACTCCTTTCATAAAGCTGAGTTCAAATCCCACATCCCAATTTAAAAAAAAAAATCATGTTATAAGCAA CACTAACAACAAATCCACTTTTCTGTCAGCCCTGCCCACCTCCCCATCTTCCTTTCTGAGACATGCAGCATTTCTCT GCAACAAGCAAAATTTTGTATGTGCAAACACACCCTCCCACCCCAAACTTTATTTTAGTCTCTGTGTCTTGGTTAAG GTGTCTTCCTCGCAGTGCCTGCCACCTTGCGGTCGAGCCCAGCTGGCAAATGAAGCTGAGCTCCAGTTCTGCCTGAT GCACCACCCCATGCCTTGGTCTTCTGCTGGAGGCTGGGGGAGGTGAGCACTGGCCAAGGACCAGCCCTCTGGTTCTG TCCACATCGGCTTGATGCCCATTCAGGGGTTGCAAGTTCTCCTGGTCTGGATCTATTAGTTGAAGAAATTGCCACCT CGTAATTTCAAATTAGTGGGAATTAAAAATTTAATGGGATGTATGTATTTTTTGGATCATATGAAACTATGTTTTAT GTGGTATTTAATGTGAGTTTAAATATGCTTACTAAATATGACTTTTGTTAATATCTCAGTTGATGCTCGTGCGAAAA AAGTCTGCCTTTCTACCTTTCTTTCCTACCTTTCTGCCAAACCCAGTCCTTTTACTAATATTTTAAAGTATTGATTT GAAGCAGCAGTGTGGTTGCTTCTGTCTGGGTTTGAACAAGTAACTGAATTTTAATTCCTGAGACCTCTTACATATAG CAATTGTCTTGGCTGTTGTAGGAAGGCAGCACCCTCATTCCTCTGATTTAGCTCTCTTTGATTCTACAGGAAGTTGT TGCCACTGTCTCTTTTTATTAAACTAAATGCCAGAATTCTTTCATGTCATCATAAGATGTTAATGCATATTGCTTTG GGGACCAAAGTCATACAATTAGAAACCACTCAAATGCTTTTTTATTCTAATTCTGAGGTTACCAATTCCTTAAAATT AGTAAATTTTCACTGATTTTGCAAGCCAGTTGTAAAAAAAAAAAATAGTATAAACTCCTCACTGACGGTAGTTTGGA AGCTGCATTGTTTGGATGTGATGTTTCAGTGCCACACACGTTAGCGGTGGGAACTGGAAGCCAGTCACGAATCGGCT ATCTGCTTCAGGGATCTGGCTTTGCACTCCTGCCCCTGCCCCTGAAGTGGCTCTAGGGACAGCTGTGGTTTGAGTGG ACTCTTGCTCCTTCCTGACCTCTTAATGGCCACATGGACCCCCTTGCTTATCTTTGAGAGCAGGGCCTTGATGGAAA TGCAAATGTGTTTCTCTTTGGTAACCCAGATACCAAAACCAAGTGCAGTCCTGGAAGGTGAAAAGAGTTTCCAAAGG AAAAATAAAAGACACATTTCTAGGAAGCATGGGAAAACCTTGGTGGCTTTGTCCTCTGGTGACAGCAGCCTGGGGGA GGTGGAGTAGCTGGGATGCCCCCAGCTTTCGCCTCCCCTGGGCTCTATCCTGCATGTTCTTGGTCCATAGCCCTCTG TGGGCCATATCTTTCCAGCATCAGTACCTGTTGGGGTCCGTGCTAGGAACACAGCAGTGAGAGATGAAGATGTGGCC TCCCTGACCGCCTGGAGTCCACAGTGCAGGGGAGGAGGGTGACTTTCAACAACTAATGAACAAAGCGTGGCTCAGTC GTAGCTGAGAGAGGCACTGCCCAGGAGAAGTATGAGGCATCGTGGGAGCATTTATCAGAGTCAGAGGAGGCTTCTTT GAGAGGTAGCTTTTAAATGAGATCTGAACCACGTGGAACAAGTAAGAGCTCACGAGTGCCTGTGAAGAAGGACCCCA CCTTGCACTTGGGGGACCCGAGGAGGACCTGTGTGCTTGCCGTGCAAAGCAGCAGGACAGAGGACCCAGCTGAGGGC CAGGCGGGAGCAAGGGCCCAGCACACTGGGAGGTTTGAGAGGGTGTCAGGAGGCCATTTGGATGGAGAAGCCACGGG CAACCAGAGGAGTTTCGTCTCGAGTCCTCACCCTGGCTGTAGTGTGGTGAGCACATTGGATTTGGAGAGGTTAGGCA GATGCAGGCAGGCCAGTTACTGGACCATCATGGTGGTCTAGGCAGGGAATGACTGGGTTTAGACTAGTAGAAGGATG ACAGCAGTGACGTCTGTAAGTGGACAGATTGCCGGAAGAGTTAGGAGTGAAAACGAGGACTTTGTGATTAAATAGGG CCAAAGAGGAGGATAAGGCAGGCGTGGGCCCGTGTTGCTGGCATGTGTGACTAGCAGGAGGGGAGTCCCCATCAGAA GGGGAAACAGAGCAGGAAGCCAGGAGTGGCGGGAACAACGCTTCAACCTTGGGCCTGCTCTTGTTGAGGTCCAGGGA ACCAGTGGGTCGGATCCGTGAGCATGCAGGCATGGAGCTCAGAGAAGCAGCATGTGTCAGAGATATGGCCTGAGAGC CACACACCCACTGCTGCTGCTGTGATCTCCTTGGGTGAGAGCATCAGGGAGAAGAGAGAAAGACCCAGTAGGTGGTG TCGCCAAGGGAGCAGGCCGTGGTGACAGAGGCTGGTGGAACCATCGGGAAAAAGGGTGCCAGAGCCCATTTCCCTGT GGCAGCCACCACCCCCTCCTCACAACACCCACCAGGTCCCTTTTCCTTAAATCAAAACCTGGTGGATTTCAGGGGCA GGCGCTGCTCAGTTGTGGCCTCTGCAAGGTCCCGGCCATGTGTCACCATTGAGTAGCTGCTACCTGGCACTGCGCCG GGCTCTCATGAGCACATGTGAGTGAGGGAGGGAAGGGGCTCCATTGGCTGAGCAGGGTGCACCCTACCTCTCCTGTA ACTCCCAGGAGTGGCCGTCCCACACCCAGATGGGGAGTCGGGGTGAAGCCACCATCCTGTGCGTGAATCAATGGAAG GCAAGGCTGGACTCACCCCCGCCTTTCCCCAGGGCATTGGGCCGGATGTTACTGAGCCTCCTTCACTTCCTCTTCCC TGTGGCCGCCCCCCGACCTTCATTTCCTTGGTAATGGGACACTTTGGCCGCTGTCATGGTTGGCCAGGCTCGGTGTG GGCGGCACCCACCAGTTCCTTCTGTGAACATACAGGTTGATGGCAACAGCGCTGGGGCCATGGGGATGACTGCGGGG AGAAGGGCTCATGGCAGAGCAAGCGCAGTGGACCCTCTCCCTCTGGCCAGTGTTCTTCTGTGAAGAGGTGCATCTCT GCGTCTCTGCGGCGTACTCTGCGAGGGTCCCTGTGGAAGTGGCTGAGATCAGCAAGACGACGTGTTTGCAGGGGTTG GGCCACACCAGGCTCTGCACGTGACATGGAGCCTGCAGCTGGAAGAACAGTCTGTTTAATAAAAAATAACCATTTGA TAATTATAATCACTTTTAAATTGATAAATGGATTCTTTTAAATAAAAGAAACCATTAAGAATGATTGTTCTGCAGAA AAAGCACATGAGGAAATGAAATTTTCTATCTCATTGGGAATTTCTTGGTAAATATGCAAGCAGATACATACATTACA TTTAGATTTTAGAAGTAAAATGACCTCATATACTTTACTTTTTTAGCACAGAGAATTCTGTATAGCATAAAGCAGTA AATCACAGATACTGGGCATTATGAGTGTATTTTTCTTTTGGAAAAAAAAACATTGAAAAAGCAGAGTTATTAATTGG AGTCCTCAAGGTCCTTATTTCAGTAAATCTGAAATGGTGACCATCTTACCCATAGCTAAGTTGGGATGCCAAAGCCA TTTCCACCATCAGCGTGCTAGTAGTAACCATCCAGGGGCAAAGCGGCTGATTCTGCAGCCTGGGCATGTGGTATGTG GGTGTCTCTGATGGGCCAAGGGAAATGACAGGCCCTTGTGCTGAAGACTGATAGGAGTTGATTTACATGAAACACAT GTGTTTTTTTTTTAAAGACAATTTTAATGCACATATTTTCATTTTCAGCTTTATAGTGTTTTATGTCTTTTGCCTTT GTGATTTAAATTGTCTTATATTATTTATAAGCATTTTCTATTATTTATAATTATTTTTAATGATTTTATGTAATACC ATTTTATTTCTAGCTTGACCTGAGTTGGCCTATGCTAACACATTTAAGCTTTCCTAATTCCCAAGGTTACCATAAAT CAGCAGGTCCCTAACTTCTGTATTCATCTCTGCCCTGTAGAGAATAGCCAACCTCAGGTTTTCGCAGCCATGCCTCA TCTCTCTGTTTCTCTAAAGGGAAGCCATAGCAGGTGCCTGGGTAATGTCCAGATCTGAAAAATCCCATGTAACCTGG AGTTGAGGGCCTGTCTTTCACCCAGTCTGTTTGTAAGCAGTGCCTCTGCCCCCACCATGTCCCCACCTGACAGGCCA GCTCAACACCTGCCTGCCTCCTCGTCCCTGCTCTAACACTAGTCAGGGCGCACAGCCCAGCGAAGTGCTCTGTCTGC TGGGAGGAGGTACTGGCTGTGATCGGACTGCCCCCCACAAAGCCCTGAGCAGGTGAAGGGTGTGGGCCAGCCGGGGG CGAAGGAACAGGTCACCGAGGCCACTGCCAGCACCACCAAGAATCAGGCCAGGTTCCGGGGGCATCTCTGTCCCCAC CAGCAGCACTGCCCTTCAGCTTTTTGATGAGGCCGGTTGTTGGCCATAAGAGGAGCGGTGATAAGTTTAGATTGTTT TGGTGTCTGTAGGTCATTACTGGAATTACACCAGCCAGCCATGAGGTCATAAAGTTACCTTGACAGATTGTGAATGC CGAGTCCACAGTAACTCTGGAGGGCTGCCTGGCATCGTCCTTCATCATTGTCCAGAAACGCTCCTCTCGGGAAGGCT GGGCGTAAAACTGAAAAGAGGAGGCTCAAGGAAAGAACGCCTCGGTGGCTCCATGCAGGAAACATTATTTCTGATTT CAAGCTGGAGAGAGAAAGGACTTTGGGGCCCCTGTTTTGGAGGCCTGGAGTGATGAGTACTGTGTCTCCAAGCTCAG AATGAAGCCACCAGTGGTGGCTGCCTTTGTTTCTGCACTTTCAGTTCCTGCAGGTGCAGGAGCTTGCCAGGGCTGCT GTCAGGGACACTTTTCCAGGCCCCGCTGCCCTGGCTGGGTTTCCTTCTCTGAGCAGGAACAAGTGCCCGATGCTGTT GCTGGTGCAGATTGGAGGGCTGCCAGGGCTGGGTGACTCCCTGCCGCATTTTGCCAAGCTTGTGTAGCTGCTTCTGC CTCTTGGAGGGGCTGTGTTAGAAAGCATGCATCACCTCCAGTTACCTCAGGCAGCAGGAAAGACTTTTAACCTACTA AACAAGCGTCTTCCCTGGGAAAATACATCCTTTGGTTGAGACTGTAGGAGACACACTGTAAGTTATTTATCTTGAAC CCTAAACTGCATGTAAAAAATGTAACCAAACCCTCAGTGCCTGGGGCGAAATCCACTCCCTAATGTTTTAATGACAC AGGATCCACTATGCTACGTGTGCACCGGGTGTAGACTTGGTCCAGTGCATGTGGTGCTAAGCCATAAGGGACTGTTA GGAAGAGCCGGGCATGGTGGCTCATGCCTGTGGTCCCAGCACTTTGGGAGGCCGAGGTGGGTAGATCACTTGAAGTC GGGAGTTCGAGACCAGTCCCGGCCAACATGGCAAAACCCTGTCTCTACTAAAAATACAAAAACTCGCCGAGCGTGGT GGTGCACACCTGTAATCCCAGCTACTCGGGAGGCTGAGGTGGGAGGATCGCTTGAACCCAGGAGGCAGAGGTTGCAG TGAGCCAAGATCATGCCATTGCTCTCCAGCCTGGATGACAGACTGAGACTCCATCTCAAAAAAAAAAAAAAGAATCG TGATATTCTTGATAGGAAGTAAATGTTAGTCATAGGATTACAAGATTTTATTCTAAGCTCTTACAAGCAAGCAGTTC AGATTAATGTGTTTTCTTAGAAAACTTGATTTGATTAAATTTCATACTTTTTATCTATTCCCCAAAATTTATGCGGT CCTTGTGTGCTTGAGGGTTCTAGGAACAAAGTGATTGGGGAAACACCGGTCCCCTCTGCTGTGGGACCCTGCGGGCT TCCAGAACTGGCGTGAGGTCCGAGTCGCTGTGGGACCCTGCGGGCTTCCACAACTGGCATGAGGTCTGAGTCCCGGT GTTTCACAATATTACTTGACCAAAGAGCGTTTTTTTCACAAACACATGAGGCAGCCCATGTGACTCCAGGGACCCGC AAAGCACAGCTTGAGAAATGTTGGTATGTTTCAATGGAAATGTATGTAATTAAGAATATTAGTACAATATTTATTAA AAGTTTCTGTCTTGAATGAAGTAATAGGATATCCTTTAGGAAGCCTGATGTCGGGACGTGCTGTGGGGTATTTTTAT GTTAGGTGTTAACATTCAGCAAACAAATCCAGGCTCTACTTTTATCCTTTAATGAAAGATTTTATTTTTTATTCAGC ATTGGTGCATTGAGGACTTACAGCCATCTTATCTTACCTCGATGCCATTGAAACAAAAAAATTAGTCATAGAAAACA AACCTAATGCCTTCCTTTGCAAGTTCCAGTCAGAAGTGTCGCATATACCAGACAACACATCATTTTTATAGTTATAG AGAGAACTCCTATGTACTTCATGGGAATACAAAAATGTCACAAAATTAAAAGGAATTTTTCCAAAATTACTGTGACT GAACAGATCATTCCACGCTCCGTTATACCATTCTGATCCTAATCCCATAGTTTAAGTGTAGAGGCTGGAAAATGTCT TCGATTTAGAAAGGAAAGACAGGCGAGTGCTGTACAATGGTGCAATTCAGTGGCCAACGAGCCCAGCAGTCGCAGAC CCCATACATTCCTCTCGTTCATATCCTATCGATTTGTGACAGCGATCACATGCTATTGTAGTACAAGGAAGAGGTTT TTTTAACCGACTGTATGACTTGTAGACTTTTCTGTGAATTTTCATTAAGGTTGGTGAATTGTACAAGCTGAGGAAAG CCAAGACAAGCTAGTTAAAATCTCCTGGCACACAGGTCAGCCTGAGTTCAGATTTTATTTCGCGGGCATGATTTTGG GGATCCCGTGCTAGATTGTAGTTGTGTTGTAATAAAGCCCCCTTTCCAGCGCTGCAGTGGGTTGTGGAAATTGTTGA GAGGAGAAACCACCTTCTCCTAGGCATCTGTATTTGGTTACCCGCCAGCACTGAGACATGCAGGCATGGAAAGGCAG ACTCAGGAAGGTTCAGGAATTCTACCGACTTTGGAGCATGCTCTCCCCCGGATCTGAAACACTGGCCCCCGTCTTAT CCACCCACCACTCCTTAAGACATCCAGGCGTGGAAGGGAAGATTCAGGAAGGTTCAGGAATTCTACCGACTTTGGAG CATGCTCTCCCCTGGATCTGAAACACTGGCCCCCGCCTTCTCCGCCTCCCGCTCCTTTAACCAGTCGTCAGGTATTT CCTCAGGTACCCGCTCTGGGAAGCCTTCCCTGACCCTCCTTTGCCCCCGTGGGGACGTGGCTGCTGCTTCCCCCGTG TGCCTGTGGCCCCTTGTGCCCAGCGCCATCGCCACTCCGTGAGGGTAGATGTGGGTGCCGGCCGTGTCTGATGCCTC CCCACAGGGGCCTTGGGCACGCTGTGTGGACGCTGCCTGTGCGTGCTTCCTGAGCGACCCCATCAGTAAAGCGTCGC CACCATGCAATGCTGCTGGCAGTCCTTGTACTGAGACAATGTGGTAGCTGCCATCCTGAATTTCCCTCTGAAACTGC TCCCACCACCAGTGCTGCCTGTGCTGTGGCCCTGCTTTGTGTCCTGTGGCCTAACACTGGAGCGCCGGCTGCTACCT GTCTGTCTCCTGTGCTGGACAGGAAGCACCATGCAGGCGGCGTTGTCGTCCGATGGTCTCCACCTGTGGCCGTAGCA CCGGGTGCGCTGTGGACTGGGACACTTCCATGTCACTCCACACGTGTGCCCCGCTGTGGCACTGTAAATATTCATTT GAGCTGGTAAGGAAAATGACATTTTAAATGCCATGATGATATATAGTGTATTTCTCATGTGAATCACGTTTTCCTTA GTATGGCTCAGCAGCACGCCATGTTTCCGTCCCATAGAGACCATGCTTCCTGTGGGGACGCCCATGGTCCTGGCTAC CTCTGCTGAAAGACGGCCAAGGTGACTCTTCAGCTATAAGAGAGATCACTCATCCTGGAAACTTGTCAGAGCAGGAG AATTCCAGAAGGATCAATATTTTTAAAAAATTCTGTTCCTAAACTTTTGCTCTGCAAAAGGTCTTGTAAAAAGAGAA CAGAGACAAGTCAGCCCAGGAGAAAATACCATTGACCTTTGAACAACACAGACTTGAACTCTGTGGGTCCACTTACA TGTGGATTTTTTTCAACCAAATGTGGATGGAAATATAGTGTTCACAGGATATGAAAACCGTGTATATGGAGGGCCAG CTTTTCATATACACAGAATCTACAGGGCTGATTGTAGGACTTGTGTAGGTGCAGATTTTTGTATCCATGAAATCCTG GAGGCACTCACCAGCATATACCGAGGAACAACTGTATTTGCAAATCCCATATCTGACAGAGGAGTTGTATTCAGAAC ATGAAACCAACTCTCTAAACTCAAAAATAAGAAAATAAACATTCCAGCTAGAAATTGGACAAACAATCTGAACAGAG ACTTCATCCAAGAGGAGATACGTAGGACCATAAGCACAGGAAACAATGTTTATTGTTAGCCAACAGAGAAATGCAAA TCAGAGCCTCAGTGAGGTTCCACGCAGAGACCACACACTCCTACAGCCACTCGGAGACCACGTGGCCACTTCCCAGA AAGTTAAGCATCTGTTTCCACAGGAACCGGAAGTTGTACTTCTGCTCTTGTATCCTAAAGAAAGGAAAACTTATGTC CACACAAAAACTGTACACAAGTGTCCATGTCAACTTTATTTGTAGTGACCCCCAAATTGGAAGCAATTCAGATGTCC TTCCAAAGATGAAAGAAAAAGCAAACTGTGGTCTATCCATACAATGGAATACTTATGAAAACAGAATAAAAAGCAGC CAACTATTGATACACGGTACAATTTGGATGGATCTAAAGGGCATTAGGTGGAGTAAAGGAAGCTAATCCTAAAAGAT AGGATTTTTTTTGTATGTAACACTCTTGAAATGACAAGCAGTGATGGGGAAGAGATAAGGGTTAACAGAGACTGGGG ACAGATGACTTCGTAGGTGTAGCCTGACAGGTGTCTTTGTGATGAGACAGTTCAGTATCCTGTCTGGGGTTTTGTGC ATGTAAATCTAAACACATTATAAAATTTCATTTTAAAACATAATTTTTAAAAGTCCATTAAAAATAAAAATACATAA GAATTTAAAATGTTTGTAAAAACTGGTGACATCAAATAAGATAGGTAGTTTAGTTAGATGTATTGTATCAATGCCAG CTTCCTGGTTTTGGTAACGCATGATTATGTAGAAAGTTAAGCTGAATGAAGGTGCCTGGGAACTGTCACTAATATTT TTCTTGTGAATCTTCAATTTCAAACTATAAAGTTTAAAAAAATTTGTGCCTTTTACATACCAGTTTTTTGTTGTTTT TGAGACGGAGTTTCACTCTGTCACTCAGGCTGGAGTGCAGTGGCGCAGTCTTGGCTCATTGCAAACTTCATCTCCCG GGTTCAAGCGATTCTCCTGCCTCAGCCTCCCAAGTAGCTGGGATTATAGGCGCACGCCACCATGTCCGGCTAATTTT CATATTTTTGGTACAGATGGGGTTTCACCATGTTGGCCAGGCTGGCCTCAAACTCCTGACCTCAAGTGATCCACCAG CCTCGGCCTCCCTAAGTGCCGGGATTACAGGCGTGAGCCACCACGCCCAGCCCATACCCATTTTTAAAAAGAGAAAA TGGACAAGAGAGCAGGGAATGGCGTTGGCTTTGCTGCTGTGAGTCTTGGAAAACCCATTTCCAGTGCAAGTGCCTGC GTAGGAGTACGGGGGCAGCATTCTTGTAATGAAAGTGAGCCCTAAAGCCAAAAGGTGATATTCCCCCTGAAATTTAG GAAAGAAAGGAAACTCTTAGGCTGGAAAAATGTGGCTGAACTTGATACTGTTCATTTGATGGTTCTTTGATTAGCCT CATTACCTAGTATTTGAACAACATAAAAAATTGGTTAAGAATAGCCAAAGAGAAAATGCCCGGGTGAGTGCTAGGCG TCCATACACATGGCAGCTTTTAACAACGTGCAGCCATGAAATATGGAAAACTTCCTGACGTTGATCACAGTTGAAAC TTCATACTTTCTTAATTCAGCCTCTTATTGACAGTAATATATTTTAACTCAAGAATGTGTTCATTCAGTGTATATTG ACTTGGCTTGTTGAAATGTTCACAGTACATGTGAACACTGGGTTTCCTGTCATGATGACTTAAGACAGGAGGACAGC AGTGAGTGATTCCTGCCAGTCAAGGAACTTTCCCTGGAACCCCACTTGAATGGTAATAATCTGCTCCTATAATTGCT GTGGAAAAGGAAAGCCCTCTGGAGATTTTACATCTAATCATTTATTTATAAATCTGTAGATGGCAGTCTGCAGAAGA TGTGTCAGGGGTCAACTTTGTGCTCAAAACAGTTGCCTGATGTTTACATTTCACAGTGTATTTTTTTAACAGTACCT CCAGCCCCCATCATCACAAAAGGACCCTGGCTTGCTGGTAGCTGCTCCCCACTCATCCCACAGCCCAGACACTTCAT TACAGATGCCTCGGCAGAGGCTGGTCCCCTCTGTATTTGCTGAAATATGCATGTTTTTCCATCTCATTAATTCAGTC TACATACAATTAATTTTTGCCTATTAATAAACCCCTTGGTGTTAGCGACAGCTATCTAAACCACTTCACCTAGTGTG GAATGAGCAAGTGTACTTGCAAATTTCCATCCCTGGTCTGCATTTTTTAGTGTTGTATTTGTGGGTTGGAGAGTTTG GGTGCATAAATCAAGCATTGTGAAGAAATATAACTGCCGAAGACTCTGGGTTCAGACTGGCTGATGGGAAGTTTGAA ATACGAATTAGTTCCACATTAATTGGGGATAGAGGGAGATGCAGGGGGGCCATCCCAGCACGTGGTAGTCAGGGAGG GTCTCTGGCTATGGGAAAATTTGTCAGCCTTGTATGTGCGTGAGACCAACTTTCTTTTTCCACAAATACAAATCCTC TCCAAATTGGACACTTCTCAAAAGATATCTTTTCTGTGTGTTTTCAAATCTCATAATTTCTTAAACATCTAATAAAG TCTGTGAATACACGTAAGTGATTGTGTATGTGGTAACTACACCAGCCAACAGCATTGAAAGGAAAAAGGAAGAAAAC ACAAAAATCAGAATGGAGCCTTGAAGTTGAGATGAGCACCACCTAGCTCATTTAGAGACATCCACAGGAAAGGAGGT TCCCCAAGACTTTTGTTATGCTCCAAAGCCCTGAGACAGATCCATGTCACAAGGACACCTTTCTCATGGTTCAACAT CACCAGCCAGGAGGAAATGCAGAATTCTGTATAAATGTTAGGAATTCTTAGATTTCTCACCTGGATTTGTAAAAATT GCAGTTCATGGCGAGAGACATCCGGGAGATCCAAATGCCATCCTCCTAGAACTTGCTGGAGGAACCAGATTCTTTCT AAGCGATTTGCATCTTGATGTGATGCTACAGTTCTATGACAGGCCCGAGTAGCTGATGAAATTCTACGTGGGAAAAG CTTTTGAGTGCCTGTCCTGGTAAACCTGTGACAGTGGCATCACATAAAGGTTTCCAGAATGTTCTGTGAGGAGATTG AGCCAATGTCGTTGGTCAGCTTAATTGGAAGCAGGTGATTGAAAGTCAGATAAACATGCAAGATTTGCTGATTTTTA AATATGCCATATGTGTCAAATGTATGAATTGTGATGATGGACAAGAACACTTTCGCAGCTTTAATGGGAGCATGTAA TGACTTGAACAAGACTGCTTAGTTTTGGTTACTGATGATCAAGTACAGAATCTTGCTGGTGTCTCCAAAACTTCCTG CCTGGCCTGGGCAGCTGTCCTGGCTCCCTCTGGCCAGTTCTCTGCCAGGCAGCCAGTGTGAATCTGGAAGGGGCTAG TCCTGTCTTCCCCTCCCTGTCTAGTGCCTTTAACGTCTCCGCTGAGCCTGCCACCTCCTGGCCTCCTCGACCCTCCA CCTCCCCGAGCCGTGGAGGTGAGTCCTAGACCTTCTTCTGTGGGCTCCTCTCCCCGGGGCCCTTTCGGCTCAGCCCC ACTTCTCTTATTCACCCACTTGTGTTCTCTGTAGCTAAATGATACATAACAGGAACTCGTTAAGTAGCTTGTGCAGT GAATGAGTTAATCCTAGGTGTTTTCAGAACAGTGAATTTCAGGTTCCTGACTCCGCTTTGAGCTCATTCAAAGAAAG TCATGTAAATAAGACCTAATCATTGCAAGAAGTCTAATGAAGAAACCATTCATTATCTCTGGAGATGGGCAGGTACA CCTCACAGAAAGTGAAGTCTGTTGCTGATGAATAGTCTGCATGCCCACATCTTCCTCTGGGTTTCTAAGTAATTGAA TAAGAAACGTTTCTATTTATATTCTTATTTATGTGCAACTGTATTTAATTATTCCCTAGTTTTGAGGATTGGCCAAT AAACCTTTTCTTCCCTTTTGCCATTGATTATTCTTAAGTTTAAGACAATGCCCAGTGGCAGATGTGCTTAACCGCCA TCCTTCATCTAGTTCATGTGACTTTTTAATGTCAATTATAAAGAAAATTACTCCTTTGGCTTCAGAAGTTTATTTAA ATCTCTTCTGAGAAAATTGGCAGCTCTTCGGAGTTAATGTCCAGAGCAGCAGAAACAGAAACTGGAGGGCACAGACT GGTGGATATGTGAACAGAAGCACTTTTTTTAAACAAAGTTAAAAAAAAAATGTTATGCTATTGCTAATAAATTGAGT CAGGGGAAAGAAAAAAAAAAGAGATCCCTAAATCAGCTTCCAAGCTGCCATTAAGATGGGCAAGTAAGATAATTCCA CATAAATAAGACCAGAGGCCTTGTCTCTGTTTTTATTTATGTATTTATTTTATTATTTTTTTGAGACAGAGTCTCGC TTTGTTGCCCAGGCTGGAGTACAGTGGTGTGATCTCCTCTCACTGCAACCTCTGCCTCCCGGGTTCAAGGGATTCCC CTACCTTAGCCTCCCAAGTAGCTAGGATTACAGGCGCCCACCACCACATCCGACTAATTTTTGTATTTTTGGTAGAA ATGGGGTTTTCTGCTGCTCTCAAACTCCTGACCTCAAGTGATCCACCCACCTGGGCCTCCCAAAGTGCTGGGATTAT AGACATGTGCCACCGTGCCCGGCCTGCCAGGGCTGTTTTTAAATGAGGATTTCTCCATTTAAGGAGGTCACCTGTCC TGGACGGTATTTCCTAAATGCCTGGCATAGGTGACATCATGCGAACAAGTGATGGTTCAGAATACAGAAGTCATGAG TTTGAAGGCTGTGATAGAGGAAGGCCGGGAGAGAAGTGAGAACAGAACGTGCTTGCCCTGGGCCTGCAGCTGTTGCC AGCATCTTCTGTCAGCTGATCTGGGCAGGGCGTGGGGGTTCTTCTCAGGCCTACCTCCTAAGAACAAAAACATGACT GACCACGAGCCTGTCCTCAGGATGCAGCCTCCATCCTTGGTGGGCACAGCTGAGAGAGGAAGTTCTGAGCTGCTTTG GGGTAGGGCTCTGTTGCACTCACCCTCCCATCTCCTTAGTCGCTGCAACCCTGTGTCACAAATAGTAGGTTCTCAGC AAGTGTGCTGAACCAAGGGGTAAGAACAACGTTTTTATTTTTTTATTTAATTTATTTTTATTTTTTGTTTTTACATT TATTTTTTAATCCATTTGGAATTTTTCTTTTTGAGATATATATAATAGGGATGAACTTTTATTTCATTTTTCTAGAC AGGTGTACAGTTACCTTAGGTACATATATTGAATTGTTATGTCTCACCAATGCACCACAACGTAGCAGTCTCTCATA GCCCGACGTATCACCCAAAGTTCTTTGTCTCACGACCAAGAAAGTTAAGGAGCGCCGACGCCAAGGGTATCACTGGA CTGAAAATTTAATAAGCAAAAGAAGAAAGCTCTCTGGTGCACAGGGGGGACCCAGAAGAGGGTTGCTGTTTTTACAG TTGAATGCAAAGGCTTTTATAGGAAACTGATGAGAGCTGGGCATCTTATTTGCATACGGTGCAAATCTGTGGTAGCT CCACCCCGTCCTCCTAGTGCACACGTAGGCCCTTAGCTTGAGTTACTCCATATTGCTTTGTTTCCCTTACTGTGCAT GTGTCAGGGGACAAAATTTTTCATTGTGGGCATGTCTGGGAAACTCACATGTGTAGCCCTTTTTTTTTTTTTTTATA CTTTAAGTTCTAGGGTACATGTGCACAACGTGCAGGTTTGTTACGTATGTATACATGTGCCATGTTGGTGTGCTGCA CCCATTAACTCATCACTTACATTAGGTATATCTCCTAATGCTATCCCTCCCCCAGCCCCCCACCCCACAACAGGCCC TGGTGTGTGATGTTCCCCTTCCTGTGTCCAAGTGTTCTCATTGTTCAGTTCCCACCTATGAGTGAGAACACATGGTG TTTGGTTTTCTGTCCTTGCAATAGTTTGCTGAGAATGATGGTTTCCAGCTTCATCCATGTCCCTATAAAAGACATGA ACTCATCATTTTTTATGGCTGCATAGTATTCCATGGTGTATATGTGCCACATTTTCTTAATCCAGTCTATCATGGAT GGACATTTGGCTTGGTCTTTGCTATTGTGAATAGTGCCACAGTAAACATACGTGTGCATGTGCCTTTATAGCAGCAT GATTTATAATCCTTGGGGTATATGTCCAGTAATGGGTTGGCTGGGCCAAATGGTATTTCTAGTTCTAGATCCTTGAG GAATCGCCACACTGACTTCCACAATGGTTGAACTAGTTTACAGTCCCACCAACAGTATTAAAGTGTTCCTATTTCTC CACATCCTCTCCAGCACCTGTTGTTTCCTGGCTTTTTAATGATCGCCATTCTAACTGGTGTGAGATGGTATCTCATT GTGGTTTTGATTTGCATTTCTCTGATGGCCAGTGATGGTGAGCATTTTTTCATGTGTCTGTTGGCTGCGTAAATGTC TTCTTTTGAGAAGTCTCTGTTCATATCCTTCACCCACTTTTTGATGGTGTTGTTTGATTTTTTTCTTGTAAATGTGT TTAAATTCTTTGTAGATCCTGGATATTAGCCCTTTGTCAGATGGGTAGATTGCAAAAATTTTCTCCCATTCTGTAGG TCGCCTGTTCCCTCTGATGGTAGTTTATTTTGATGTGCAGAAGCTCTTTAGTTTAAAGACTTCATGTCTAAAACACC AAAAGCAATGGCAACAAAAGCCAAAATTGACAAATGGTATCTAATTAAACTAAAGAACAACGTTTTTATTCTGTATC ATCAAGGAGGGAACCCAGCACGCCTCTATCCTTTTAAGAGACACGGCAAGCATCAGACCTGAGGGACATCATCATTT CCTTGCAGCTCAGAATGCACTTAAGTGTCATGTAGTGGGTTTTCTGTTTATGGGTTTGTTTTTTTTTTAGAAAATGT GTTCTTTTCTATCTTTTGTTTGTTTTTGAGACAGGGTCTCGCTGTGTCACCCAGGCTGGATGGAGTACAGTGGCAAG ATCTCAGCTCACTGCAACCTCAGCCTCCTGGGTAGCTGAGACTACAGGTGTGCACCACCACGCCCAGCTAATTTTTG TATTTTTTGTAGAGATGGGGTTTAGCCACTTTGCCCAGGTTTGTCTCAAATTCCTGGACTCAAGTTGTCCACCTGCC CCAGCCTCCAAAGTGCTGGGATTACAGGTGTGTGCCACTGCGCCCAGCCCAACCCTGTCCTTTGTTTCTTACAATAT TCAACAGTCAGTCACTTCTAGCTTTCACTTTGTCCCATAAAAATATTTCATTGAAAATTGCAAGCCATGGCAGTTTG CAGTGTTTCTAAGCTGAGGAGTTCAGCTCCACACCCAACAGAGCTCCAGCTTCTGGTTTGATTCTTAGAGATAGGAG TGACTTTTTTCAATCATGTACATCATAATTAGGAGGAAGGCATTCAGTCTTGAACATGGGGACAAAAAAGTGCTGGA GATAGTAGCTTGAGAGTTGCAAATATTTATTTTGATTCTGACTTTAATTCTTAAAAATGTTTGTCAAGAAGACAGTG GTGTGATGGAGCAGGTCCTTCGGTTACCACAAGCACACACCCTTTCTCTGTAGTCTTATAGCTTGTTTTCTTGGTCG ATTGTGCCAGCTCTTATAAAAATATTATGCTCCCAGGCAAAGGGCCATTTCTGTTGGAATTGTTGAGAAGATTCTTG GCTTAACTCTTGATCCAACTCTGATCAGCTTGGCCAAGACTAGACTTGACAAGTTTGCTCTGAATTGCCTCATGCTG CTGTGTGGGGTTTCAAATCATTGGCTTCCCAGTGCTTTCTGGGCTAGACCAACAAAACAATGCCTGGGAAAAGAGAG ATGATAGGCCGTGATAAAACTAAAACTGCGTAATACTGGAATTAACTGAGTTGGCCATGTAGATGGCAAGATATTAA AATTGAGTCCTTGTGTGCCAAAGTCGTAAACTCATTCAGATAGGCCCTAGGTCTGTGAATGTTTGTTGTTGAATCCT AGGCTCTGGGTGGAACCGAAAGCTGAAAAACATGAAATTCCCCCTCTGTCCTTTGGATCTTATTCAGACAGTCATTT CCTAGCTATTTCAAGTTTTGATCAAAACAGACACGCAGTAGAAGCCCAGTCTAAAACATCGGTAGTTTATATCACAT ATTTACTTCCATGTGTCTTAAGTGGTTAAGTTGTAGAATTATGTCTTTTAGAGAATGGATGAAGAAGTCTAGGAGTA CAATAGGCAAGAGAATATAGCAAAGTAGTTATATTTTGTCAATTTTAAAGTAAATTCTGAATCATTTTAAGTATATT CAGTAACACCCGTTTGCCCCCACCTTCCTTCCTTTGAGGTTTTCACTTATTCACTCATTAATAATTCATCAGTCTTT GCTGTTTGTGGCATGAGTATGTACAAAAGGCACTGCGGCGTCCATTGCTGTGAGTAATATAGAGATGTCCAAAGGTG TGGCTTTACCTGTGGAAACCACAGGACCATACCCAGAGCATTTGGAGGTGCCATATGCTGCTTAAGGGTGGCACATG GAGGGGAGGCTGGGCTGCACGCATGCTGGTCCTGAGGTGACCCTCTGGCCGCAGGAGAGCTGGAAAGGTGCCAGCCG TGGGTGCCATGCACTTGTCTGAGGCCAGGATGGGGAGACCACATTCCAGGTACAGGGATAAGAGGAGGAAAGTCCCA GGAGTGGGAAGGCCTGGGGGAAAAGGGGAGAAGGTCAGGGGGAGGCAAGTTGGCCAGATCGGCTGGAGCTTGGGATT CAGCCAGGACAGAGCCTGAGCCCCAGGCCAGACTGCAGGGCTGTGCCCAGCACTGGATGCAGAGTTTGGATCTGATT CTGAGTATGTGGTGATGACTCGGGGAGGCTTGTGAGCAGGATGGTGACAACCAGGCAGGATTTTAGGAAGGTTTGTC TGGTTTTGGCAGGTTCTCTGGATGTGACTGGAGTGTGAAGAGGCCAGTTTAGAGGCTCTGGAGGTTGTTCAGAAGTG GCAACAAAAGGGAATGGAAATAAGAGATGTTGCAAAGCAGAAACCAACTGACATGGAGTGCGGGAGAACTTGAAGGC AGCAGTGGGAACTGGGAGCTGTTCCCGGCGTGGGGAGAAGGCTGACGTTGATGTTGCCTAAGGAGTTAGAGGTGATG ACCAGGTTCCAGGTGGCTGATCTGGATAGGCAGCTGCATGGGGCAGGAGCCCCAGTGTGGCACAGCTGTGTCTGTCC CCTGCCGGTCCTCAGAAAGTGACACCTAACACTGTCCCAGTCCACACAGAGCCTTGTCCAGTAGTCAGGGTGAGGTG AGTGGAAGCAAGAAGAGGAGCTGGCAACAGGACTTGGGAGAGGCGGAGAGCACAGCAGGCAGCTGGGCCCAGACGTC AGCCCCAGGCAGTGTCCTGAAGCAGCGTGTGCACCTGGGCAGTGCAGGCATCGCGAGTAAGAAGAGGGTGTTAGCTC GGATAACTGGGGCTCCTGAGCAGCCTTCAAGAGGACAGGTTCCTTGGGGCTGTGGGTTTCAAATAACATCATGGAGG AGGGAGCAGAGAGGCTTGAAAATAAACGGCGGTGGCCGGGGGGAGCACAAAGAAGCCAGGCCTGGCAGTGGCACAGG TGTCAGGGGTTTCATGAAGGAGGGGCCCGGGAGTGTGCCTGCCACATGGACACTGGAGCGAAGGTGCACCCTGAGGC AGCCCACAGTGAGGAATTCAAACGCAGCTTCCTGGGCTGGATCCTGCAGGGCCTGAGTTCTCTGTTCCAGGGGCATC TTCATCTGAGGATTTGGGGTGTCCGTGCCCTCTCCTGGTTGAAAGTGTTAGAAGCCACGTTGGAGAGGCCACCACTC AGGTGCTAAAGTTAAAGCTCTAGAGTTTGGTGCCTGCCACTTAGCTGCATGGCCTTGGGAGTTATTCACTCCTCTGC ACTTTGGTTTTCTCGTTAATCAAACTGGAATCCAGATACTCACCTCCTACCTTGTTATGGGAACTAGACGTGGGCGG TGCACGTGGGGGCACCGAGCACAGTGCCCAGCACCCAGTGGGTGCTCAGGGGATGGCAGCTGCCATGATTTTTGGGA CTGAATGCTTTGATACCACCCAAATATATGGTCTGAGGTGGTTGTAAATAGAATTAGCAGAGGAGATGATACAGACT CAGAGTTGAATTAGGGTTTGAAGAAGGGCTTAGGTCTGGAGCGTTACAGGTGGGAGGACCAGCAGAGAGTGGTCAGG AATCAGGAATGACAGGACACACCTGGGGAGCCTGAGCGGCGGCTTCCAGGCAGGGAAGAAAAGCTCGGGGCAGTATG AGTCCAGACCAGGGGCCTATCAGGCGCCTTCTATGCAGGGTCAGGTGGTAAAGAATTATCTCAGCCTTAGTGGGCTA GAAGATCTCTGTCATAGCTATTCAGCTCCGTTGCTTTCTGAGTAAACAAATGGGCGTGGCTGTGTTCCAATGAAACT TTATTTACAAAAACAGATGGTGGGCCAGTTTGGTATGTGGAGGCATGTTGACTATGGACACCTTGAAAATCCCATGT GGCAACAGATATGAGCCAAGTGGTGACGTGGAATGCCCCATCGTTGAGGGATGGGGACGCAAGGGCAGCGAGGAGGT CCTGTGGGTCCGGGTGTGGCGTGGGCCCAGCGTGGCCATGGCATCTGGGGCCTCAGCTGCAGGGTTGCAGTGGAAGG AAGGGGAAAGAAGCTCAGAGTGCATTCTGAGGGTCAAACTGGCCTGTAAGTGACAAACTGAGGTCACTTCACATGTA CATGGAGGGAGACAGGCTGTCTTTGGAGCAGTGTCCATGCAAGCCTGCCTGGAGGGAGCTGCCACCCTCAGCTGCAG CCCCAGACCTCGCGGTTGCTCTCATCCGTCGTTCCCAGTGTGGGCAGTGACGCAGCACCACGGTAAGTTCTGTAGAG TGGACGCCACTCGCTGGCAAAGCCCAGCAGGGCAAGGAATAAGTGTCACTCAGTCTTTATCCAGAAAAATCCACCAA TGTACTTTTAGTGCAGTATTAACACATGGTAGAAAATGGAATTGCCTAGAGGATTTTAATCTCTGATGGATTATTCT TGTACCTGTACCTGTCTGATGGTCGTGTAAGTCCATGTTAACAAACCTGGGACCACAGGAAAGGAGAACTGATGCGA ATACAAACAGGTGGAGGATTTCCAGGCGTCAGTGGGGTCTGGGAGACTTTTCCAAGAAGTGTTTTCATTGCAGTTAA CATTTTTAAAGATGAATATCTCACTATTATAAAGGTCAATGCCACTTCTGTAATATTAATTTGCAATTGAAAGGTAT TTTCTTTTAAGAAGTCCCCTTTTTCAAGTTTCTCAAACTGGAATTTTCTTGAGCATCTAAGAATCCTTTCCTTTCAA AAGATCCCCAAGTTACCCCGGTCTAAGCTAAGAAGGGTTCAGCGTTGGCCGGAAGCATAACTCTAGCTCTCCAAGGC CTTGGGCTGCGGCAGGGTTTGCATGTCCAGGCAGTAGGTGGAGGCTTCTCTTGGTCAGTGTGTCTGCTCAGGGGGAG GCCTGGCTGGGGAAGCGGGTAGCAGTTTGGGACCAGAGCTTAAAAGAGAAGGAGAGACCGGTGCGGTGGCTCAGGCC TATAATCCCAGCACTTTGGGAGGATGGGGCAGGTAGATCACAAGGTCGGGAGTTCGAGACCAGACTGACCAACATGG TGAAACCCCATCTTTACTAAAGATACAAAAATTAGCAGGGTGTGGTGGCGCACGCCTATAATACCAGCTACTCAGGA GGCTGAGGCAGGAGAATCACTTGAACCCGAGAGGTGGAGGTTGCAGTGAGCCGACATCATGCCGCTGCACTGCAGCG TGGGCGACAGAGTGAGATTCCATCTAAAAAGAAAAAAAAAAAAAGTGAGACTTTGAAAGTGATGACAGTTTCCACAG TTCCCATGGGCAGGTGGCTCAGAATGAGGTGGCCTGAGGCCCACAGTCTGTCCTTGGGGACTGGGTGGCCCCCACGT CCAGGCAGCAGGGAGAGGACGTGCTGGCTGCCAAGATGGGGTGGGAGAAGCCCAGGGCATCAAGTGCCACGGAAATA AAGCTGGTTGCCTTAGTTGAATGGCGCCTTTGAGTTCCCAGAAGCTCTTCTCAGCGGGAGGCTCCCTGTCTTTCACC TCTGCTCTTCTGAGGTACCAGTTGAAGGGTTTGGGGGGTGAAATCAAGGCATAGGGCTCCTGGCAACACTACCCCTG GCTCCGGGGTTTGCATGCCGGGGCTTCTTCCTTGTCCTGGCTGTCAGAGAACACCTGGAAATAAATGCTGCATTGTT TTGCTGGAAAATCCTGACACCACCTCCTGCCTTCCCCTGGGAGGGCTGAGACCTGGCCTAGGCATGGAGCTTACGGT CCACAGACCTGCCTGCCTGCAGCATGACAGCCTCCCAGAGGGCTGTATTGATTTTTGAAGAGGAGGAAAAAGGCATA CAAAATTGGACTTGCCAGCCTTCTTTGAGTAGAAATTGGTGCTTCCTAAAGGGAATCTTAATTGGCTAGCTTCGAAG CTAAAATGTTTTCTCCCCCTTCCCTTCTCAGAACTCTCCAGCCCCCCTGTTTGTTCTTGTGAGACTTTTCCAAGCCC CAGTTACTTTTCTTTCCTGCTTTGGAACATAATTGGATTCTTCGCTGGGTAGAGTTTTGCTTGGAAGACCTGAGGGT GTAGCTGAACCAAGTTTGAGCACCCTGGAAGGAAGACAGAAGATGCCGGGAGACCAGCCAGCTCAAAGGAGAAAGGG AGGGGGACAGCATCTGAACTCTGGCCTCCAGCATGATTTCATCCTTTAAACTGAAAACCCTGAGTTTCAGTGCCAGC GACGACACTTGTTGAAAAAGTACAAATGTTTTCTGCTTCTTGTAATCTACTTCTTGCAAATATTAGTACTCTGGAAA AAAGATTTATTTTAATAATGTGAGTTTCAGATTTTTTTCTGTAGTTAACGTTGTTTTATTACTCTATATCCTTGCCA GAAAGCGTGTTTTAAGAGTTTCAATTCAAAATTAAATATGACCTCAGTGTTTTAAAATACAATTATATTAACTGTCC ATTTAATAGTTGCTGAGGATTTTTTTGGCCACTGATGGATAGTCAAGATACTTGTCTGAGATTAAATGCAGTATTCT CAACTCTGCTAACCAGCTAACTTGTTCCAGCTGCAGCAATCAACAGGAAAAACAAGGGCTTTCTATGAAGTGAGCTG GAGAAAGTTGTTCCCGGATAAAATTGCAGTACTATTCTATAAGCTGTGCTTTTCTTTTAAAAACTCCACTGTGTTTC ATAATAGGGAACTATCTATTATTTATTTAGAACTGTACAAACCAATTTACAAGACAGGTAATTTAGTGACAGAATTA TTTATTAATGAGCCCAAGTATACAAGTTAATGGGACCGAGCCCCGTGGAGGTCAATCCTCATATAATCCATGCCTTT TGGAAAGAAAAGTATTGTCTTCAGAACATTTAGTTATGATTGTTAAATTTAAATTACGATATATTCTCTTGCTAAGA CAAAAAATAATTTTTTCTGAATTGCATGATGACAGAGTTAGTCTGTAAGCTGTGTGGAGATGAATTGTGTCAATTAG GGGTAGGTACATCTTAGCCCTGTTGGTACCAAAAGTATTATTTCTCATTGCATGGGCTCACAGGAGAAAGCAAGCCT GTTCTTTAATAGAGACGACAAATCAGAAACCGCTGCCTGCCTGGGAGATGGAACAGTCGTGCAGGCGAGGAGCTCAG AGGGCGGGCGTGGCTTCCCAAGCTCTGGCACAGCCTGCAGGCCTTATCAGGTGCTCTGCTTCTGGGGTACTCGGCAC TTTGCTCTTCCGGTGGATGGGGGAGGGGGGGGCTGTTCCATGTGACAGCATGTGGGCTTGTCATGATTCGGGGTGAC CACTGGCTTCCATTTCCCAGGAGTGGCTTTGTCCTCGCGGTCTTACGGTATGTTTCCAGTAGATGAGAAGGCATGCC TACAGACAGGGCCTGCTGGTGTCTGCTGTGTGTCATGGGGGGAGGCAAGGTTTTCCTCACTGGGACCTCTTCACTCC CCCTTTATTAATGACCCCCTGGCTCCTCCCCAGAGGCCCAGAAGCCTAGAGGAGAAATGAGGGTACAGCATAGATGC CTTAGTGTAACGAGTGGGGCTGCTGGAACTCAACAGACTATTTACAGTTACAGCATGAAGGTGGTGCTTGAAGGATT TGCTGCTTCGTTTCATTTTTCTCATTTCATCTCATCATGAAGTCGATTGCATTATATCAGGGGGGGGGAATGGAAC AGACACAGAGGAGAACCCACCAAGTTCTGTGTCTTCAAACCTGCAGCATTTCCAGGTTCCGACTGCAGAGTCCTATC AAAGTAAGCAAAATTGGAAGTGATGCCCATTTCCTGGGCTTTCACAAACTTTGTTATTCTGCAGTCTACCACAAAAT CTGAAAAGGCAGAGAAATGTAGCTTTCACTGTTTAGAAAATCTTTGTGAGTTTTTCTTCCCCCTAGGGAATGGTTTT CAGCACATTATTCATTGGAAAAATGAAATCTGAAATGTAGGTGTATTTTCAGTGCACGTGCTCCGGAGGAGCCTCAG CACCCTTGTGTCTTGTCTACCCATTCGCTCAGTGGGCAGGGATTGAGCACTGGGAATGTGCTGGGTCGGAGCCGAGG GCTGGAGGCAGAGTTGGGACCAGCCCCAGGATGGTGCCTGGCTGTTTGTCCCAGGGCTCACTGGAACAGCCCTCCTG GGCAGCCCTTGGTTGGACTTCTTTACGCAGCCCAGTCCACACCTCACCTGTGCATTCGTCAAGCCATTGAGTGCAGT GTTGTGGCCTGAATCACTCCAGGCCTTGCTTTGGAGGGTTGACAGTAGGGGACACGGCAGGCCGGGGGCATTCTGGG ACCCAGGTCTCCTGCCTATGTGGAATACTGATGTGTGTTGTGTGGACAGGTCACTGATGTGTTTAAATGTAAATCTG ACCTTCCTTTAAGAGCAATTGGGCAGCAGTCTGGGAGCTTGAGACTGTATGATGTGGACACCTAGTATCTCTTTCTG GAAGCTTCTAGTGTATAATAGAAAGTAGGTTGGTTACTCATTCCCATTGTCTTTGAATAATGGGGCAGTGGGCCTGG ATTGTTGTCCTGTGGATTGGGGAAGAGATCATAGAGATCATAGAAGTAACCTTAGAGTAAAAGAAAGTAAATCAAAA TGCTATTCTATGTAGAATATATGCCATCAAGGGCAGAGCATGTGGGCGTGTGGACTTGTCATGATGAGGAGTGACCA CTGGCTTCCATTTCAAAGAGGAGAGCTGGAGTGCTTCTATTCCACGGAGATGCCCCAGACTAAGGTCTTTGGAGTCA GGCTTTCATTCCATTCCTGGGTCTGTCCCTTATGCGTTTGGTAAGTTGTTATTACATAACCTCCGGACCTCAGTTTC CTGTTTTAGAAATAGAGATAATGGTACCTCCCCCAAGGGTCTTTGGGAGGAATAAATAGAACAGCTTATAATGGAAT GCCTGGCACATGTTAGGCACTCAGTAAATAGCAGCTGTGCAGTGTTGTGCGAGAGCTTACATCGAGCTGTTTTCCCA GAGTGCATTGATCCTCAACCGTGTTCATGTACCCCTGCCCAAGGTGCCATATTGTGTAATGGAGATGTGACGTCGAG GCGCCATGTTGCCTGGAACTTCTGGAGTCAGCCACCTTGCCTGTGGCTGACCTTGGCTGTGTTCCTGAGAGAGTGAG GATGTGAAATTGCATGACACAAAGTCGGTCATCTTGTGCCCGTCTCATCTACTCTCACTGGTGGTTCAGTTCTTCTC TGCCCCTAAATTCTTACATCCCAGAGCAGGTTCTTGGTTGAGACACAGGTGCTTGCCTCAGGTTTCCTTGGAAAACT CCAGTGGCACCAAGCGGTCAGTAGCCTGTAGCGGCCACTTGCACTGTTGTCCCCTTTTGTGGACGTGGACGTTGACT CTCCATGTCTGGTCTGTGCAGGAAAGAGTGAGTCCTCTCTTTCTCACTCCAGAGCCTGCAGGGGTACCCACCCTCAG CCGGGCAGGGCAAGCCAGGACAGAGGCTTTGAGGGCATCTGAGGAGGAAACTGCAAGGTGTGCAGTTTGGCCCAGCA GAGAGAGACCAGAATGCAGAGGGTACTCAGGGCAGGCGGTTGCTACTCTGGGCTGTGTGCGTCATAGCGTTTGCATG ACAGACACCAGCGGTTGACCAGATGCCCCATTTGAAGCCCATGTCCAGGCATGCAGTGTTGATCTCTGTGTATAGAT CGACGTGGCTTCTTCTCTAGGGCTGGGGAAGAGAACTGTTTAATAATGCACAGTTGCCTAGCCTCAAAGATCCTTTG AGAAATAGCTACTCTGTGCAAGTTTTCAACAATTTGTTTAGACTAATGGAATGACTCTTTAAAAGTTTATATCTTTA CAGATATACAAGCACTTTATTTCTAAGCAACTAATTTAAGCTGGGAAAGAAGAACGTGCCCTCATGTGTGTGTAAAA CGTTAGAAAGCAGCTCTGTTCACTGCCGTGGTGGTGGAAGGCATGTGCAACACAGACATAAGGCTATTTGAACTCTT TATTTGCCACATTTACAATATTAATAATAGCGTTTCTAGAAGTTGTCACATCAAGTAAAAGATCTTCATTCCAGGCT GTCTTTAGGGGCCAAATTGAGACTGGATGCATGCATTTAATAAATTGTAGTCTCACGTAGTCATTTTTGTAAATAAT TAGTTTCAGTGAAAGAATAAGTTAGCATATTGTATGCATCTAATGTTGCAAATGTACATACAGCAAACTTTGCTATT AATCAACAGTCAGGCACCGCAAGTGGGGTCTCATACATAATGAATTGTGCTTAAGCCTCATGGGCTTCTTGATAGAA AAATAAAAGAAAACACAAGTGCAGTAAGCTTTTGAATAAAATTTAACTAGATTGCTCTGCATCCTGTAATTGTTTTG GAATCTTTAGGGACCTGATCATTCATCTATAGTTTTGGGGTGTTTTTATCTTCATCTGTCAAATAGTAGAGACTTTT AATGATATGTTAAAATGTTGATAAAATACAATTTTCCTTTTAATGATTTGAACATTTGGAGGGGGGAGGTAAATAGG CTTTTTAAGGCTTTCTGATCTGCCAAGTATACTATTTGGGTTGTCTTTTAAAAATATTTTCATTATTTCCTGTATTT TTAAGAATCAATTATTTTTAAAAAGAAAAATATATATAGTCCTTATGGGGAAGAGTTGAAAATTTACATAAAGTGTT CTCTGTATTTTTGGTGGGTGTTTTAAAATACTATGGTAAATACCTTTGCAGTTTTGTTTAGGATCTGACATTAATTT GAGCCCCAGATATTGAGGGCATAGCGTGCCGGTTAAAAATCGGTATCCAGTTTTAGCAAGATACCTACGACCACCCA GAAAGTAACTTGGAACCTAACTTAATTTTAAATGGCCTCTTTCTCCTCTTCACATTATTGACATTAGTACATGAACC CGTTGGTGGCCTATAATAGGATCATAAACATAGGATTCATTTTGGGATGGGGTTGAAGGCATAAAAGCAAGGAGTGA GATTTATGTCCACCTTTATTATGAACCACACAGTAAAAGTGCAGTAAAGATCTGCTGACTTGACAGCCAGAGCTCCT CACAGCCCTCCTGCAAGCACTCATGGAACCCGTCCTTCTCCAGGAAGACCTCCTGTTTACTTCTGCGCTAGGAATGG GCTGTGCTCATCACTTACTTCCCTGTCTTATATGATGTCCTTTTGTACAGAATGCATCTTTAAAATAGGCCCTACAA CTAAACACATAACAGTCAATCAGTAGACTCACCTGTGATAAGTGGGAGGCCCTCTTGTACCAGAAAGTTTTAAAAAA AAAATACATTACTCATACCTGTCATTGTTTGTTACGTAAACTTAAGCATATTTTTGATGTTTTGTGCGTGTTTATTC ACTTCTTCCTTTCATAGTTGAACACATCTTGTTCTTGAGGTTGCGTTGGAAATCAGTAGGGTGGCGCATGAATCACT GAGAACAGCCCACGGTGTGTGGTCAGTGCCATCGTCATCATCGTCATCAGCAACGGCAGCTCAAGACCAGCAAGGCC GCGGTTGTGTCTGTCATGAGCCCGTTGGCATGCCGTTTGTTTTTTATCTTTGCCACAGTTGCACTGTTGAGATCCAT GTTTCCTATTACTAACATCAGCACTTTTAATTTTCCGTTTCTACTCAGTTTCCTAAAAGAACTATAGAATATTCAAA TAATTGCATGCGCTTTGTCAAACGTACATGGTCATAGTGCCCAAACAGTTGCTGGCCACCACGCAGAGCCACGGAGC AGTCACTGCCATGTTCCTTCGCAGCCTCCTCGGGGCCTCCATGAAACATCGGGGCCAATGGAAGGCGGAAGTGCTGT TTCTGATGTTGACCTGTGTGTTTGACCCTCACCTTCCCTTCCTGCGCAGTCGGCTGTCAACTCCAGAGGAAGGGACA GCCCTGAGTCAGTATTCTCACCACTAACCGCATGCATTTCAGATCCTCAGTAAATGATTGAATGAAATTAAGCCACT GCATTTGTCTGAAATACAGAGTCAAAAGGTTTTATTCAGCCTCCTGTTTAAAATAAAATGATCTTTATTAAGAGCAG GTAGCATAGTTTATTTCTTTTCTTCAGTCTTACCCCTGAAAGAAAACATCTGAGCTTTAGCAGGAAGTTATAATTAA ATGCAGGACAAATCCATCAAACACAAGACACTCTGCTAGAGATCTCTAAATGGGACATAATTGTGCATTTACCAGTG ATATTTAGCAAGAGTCTCCATGCAGCGTGGATAATCTTTGGCTGAAGGCATGTTCCTTCCTACATGAGCCCCGGAGT CTGCTGCTCTGCCCTTCAGTCCCGAAGCCTCTCCTGGCCCTCCACACCAGGTTCTGGAAGCCAGAGGAGGTGGGGTG CTCACTAGCTGGGTTTGGAAAATGTTGCTCATCACCTGAGCATCTGAAGTGCTGTGTGTGGCGAGGCCACTAAAAGC TTTTCAGATCCCCACCAGTTTCCGTGCAACCTCCTTCACAGCCTCATGGAAGCCTTGCGTCCAAGCTCGGCACCTTG CCAGATTCCAGAGGAACTCAGAGGAGCACCTGTCTTTGGAACGAACTCTTTCCTTGGGTGACCCTCCAGGGTGGTCA GGTAGCATTCCCAGACCATAAGATGAAAACGTCGTCCATGGAGAGAGAGAGGAGGTGTTCGGGTCACAGGAGAGGCA GAGGGTACACAGCACTCCCCATCTGGAGTGTCCCACGGTCAGCCCGATGGTGCCTCAGATGTGAGTAACTTATGAAG GACTCTTGGCCGATTTTCTTAAATCAAACAGGATTCAAGCCAAGTACATCGTGTGTTAATGTCTCTGGGGAGTAGAA TTCATTCAGAAATGGACATTTAGTGATTCTCTACTGCCAGTGGAATCTTAAGGCACCCAAAAATCTGAATACCCAAA TCTCCCAGCTGGCTGGTCAAGTTTTAGAATGTTGAGCACTCTAAACTGGGAAGAGCCTGGTAGCCGTGGTACCAGGA ACACTCACTCTTCCTGCTTGCTGCTTTCCTCATGCTGAGCTGGGCTGAGATCAAGACTCCTGGGGCACTTGCTTCCT CTACCCTTTGTCTGTGGCCAGGTGGGATTCCCACACAGTCAAAGGAAGCCCGTGTCCAACAGGGGCCATTTCCCTTA AATTTCTCTCATCACCCCCAGCTCTGCTGTGCCTCAGGTGGGCCGCTGCCACTCTTCCTTCACGTTCTTGATAATAT CAGTGTCTTGAAAGGAAGCTTCTTGCAGGTAGGGAGGACGCTTTTCCCTGGAATAGCTGCTGTGCTCATATGAGTTT GTGCTGGAACATTCTAACCACCTTTCTGTGCCCTGGTTTGGGTTGAAGGGAAGGCGATTTAAATCAAAATAAATCTG CAAGGCAGTTCATCACTGGGACATTGTTTTTCCAAGCCGGGGAAGGCGAATGGGTTAGGGCAGCTTCCCACAGACGG CAACTCCGGGAGAATATCACCGGGGCCTTCCAGTCTGCCGCAGCCCCGGGAGCTTCTACCCAGGGCAGGTCAGTGTT GTCCGGTTTGATTTTAAGGAACCCCTGGCATTGCCAACAGTTGTGTTTTTCTTAAAGAAATCCTTGAGCCAGGTCCA CATGGCTCTAGCTTTGCTGTGTTAAGGTGCCCGACGCCCTGTTGTCACATGTACACACGCCCAGCAAAGCTGGGCTG CTTCACCCACCAGCCACCATCCTCTCGTGCCAAGTCAAAGAAAGTGAAAGCCACCAGTGTCATGATATTTGCCTCGG GATTATTCTGTGGGTGACACGTGATGTCCTAGGAGAGGCCTAGGGGAGGTGCGACTCTGATGGGCTCTGAGTGTTTC CCCACCAATGCTGGGAGAGGGTCTTGTCTCCTGCTGGGTTCAAGGGATGGGATAGGAAGGGGCTGAGGTGCCAGGCA AAAGTCTTGTCTCCTGCAGGGTTTGAGGGATGGGACAGGAAGGGGCTGAGGTACCAGGTGAGGGTCTTGTCTCCTGC ACTGGCTCCTGCTGGCATCATGAGCTGGCATCCGTGGCAGCCCCCGGAGCTGCAGGATTTGGGCTTCCGAGCAAAGG TGCTCCCCCGGCAAGGATGGGGCGGACCACAGGGCCGGCATTTCAGTGGTGGGTGAGTTGCAGGGCCCCTGGGGAAG ACTGGGACGTGGCATTTCCATGAGCGTGGGCTACGGAGCCCGTAATTGTTAGTGTTTCAGGGTTTGCCAGTGGGGAA GCTTGAGGAACTCCCCCGTGATAAGCGCCGCCGTGAGAGAGGCCATCACCCCAGCTCTTCTTCCAGGGCCGAGCGCG GCAGGGAAGGAGGAGAGGGCAGGGCAGGAGAATAAATTTCATTTTACTGTAGAAATGGAAATTTGTGTCATGGCTTT AAAAATCTGGTTTTATGTGTGTGTTTAATGCCATTCATTTTAAATGTAGTATCATAAATCATTGCTTCAGAATCAAA GCTGTTTAATTACATTTCACCCATGAATAGTTATCTAATGCCAATAGAGAAAGAGTTAAAAAGGAGAACTACAGAGT AAATCAGAGGTTGAGATCTTCAGCACAAGCATAAAAAATCAATTTAAGATGAATTTTACTACTATAAAAATTCTTCA AATATCCCTTTAAGAGTGGGAACTGCTAGGAAAGCCAGCTCACATTTCAATTCCCTCAGCAGCATGAGAGAAGGACG CCCGCGTGGGCAGAGGCTTGGTGTGCACCCCGTTTTGCTCAGGGGTGATGAAGGAATCCTAACTCTGGAGTCTGAGC AGTTGCTGGGGGCACAGAACCTCCCAGCTCTCGTGGTAGAATGCCCCCTCTCCCACAGTGTACCCTCGCTTCCCTGA GAAACCCCATCTGCGGGGTGTGATGGCCAGCCAGACCCCGCTAGTCCTGAAGACTAACCCTGACCTTGAGGTGCATG CCTCGCCAACCAATCCCACATCCCAGTCTTACCCGGGGACCGTACAACTCACCCACCACTGAAACGGCAGCCCCTGT GCTCCACTCCATCCTCGCCGGGGGGGCACCTTTGCTTCGGAGCCCAAATCCTGCAGCCCCAGGTGCCACCACGGACG CCAGCTCATGACACCAGCAGAGGTAGTGCAGGAGACAAGACCCTCACCCAGCGCCTCAGCCCCTTCCTGTCCCATCC CTCGAACCCTGAAGGAGATGTCAGCTGAGCTCTTGCTGCAGGAGGTGCCAGGCCCAGGTGGCTTGATCAGGGGTTTT GTTTCCTCAGTTGAAGGTCGGGGCACATGGGCCCCATCACGCTTGCTCTTCCTGTCTGGGCAGTGGTGGTTGACAGC AGCCAGCTCTGCATCACTCAGGAGGGAGCCGGGGCTAACCCTCACTGCACAGGGCTTCCACCCCTCTGTTCGGCCGC CTTCCCCAGGGATCAGGACAGCAAAGTACATTACATATTTCTCCATCGTTCTTCTTAGATAGCATCAGTACACTGTT TATTCGGTTAGCAAAGAGCAGAGCAAGAATTGAATGACTGAAGGTGTGTCTCTGAAATTGAATAATATTTAAGCCAA GCAGAAGTTCTCCTTTCCGTGGCCAGACCTCACTAACGGGGAAGGAAATTGGGCAGTGAAAAGACAGCACTCCCATG CCCTAAGGAAACTCTGAGAGAGGCCACTTCGTTGCCTGCTCGGCAGGCCCATTCTCTGCCTGCAGAGTGAGCCCGGC TTCCCCCAGGGCCTCTTTGTGACGGGACCGACAAGGGTTTCCAGGCAGTGGCTTGGGCTGTGGAGACCTCAGCCTGT GTATTAAGGGTACTGGGCTGAATGAGGGCACTGTCTGCGTGAGTTCCCTTGGGCAGGGATCTGGGTAAGGCGGCAGT TTGGAGAAGACCGGGGCATGTTCTGTCTGGCCGCAGTGTCCACGGGGTCAGCGTAATTTAGCCGGCTCTGCTGGGCT TGGTTCACAGGTCCGACTGCTGGTCACAGTTATCCGTGGCTGTCGGTCATCCTAGTGAGCTTCCTTCTTTTTCCCTT CATTCTCTGGCTCCTTCCTTAAAACCGTCAGGATTTGTGTGCTTGGAAACCTCTGACCACCCTCCTGCTCCACCGTG AAAAACAGTGTATGGGGGCCCCTATGTGCATCTCACCCAAAAAGGTCCATTTTTAGAAAGACCCGAGGATCTCGGCA GAGACTTTTACACCCACAAAATGCTCCAGCCCAGAGACCCAGCCTCCAGCACAGGACAGCGTGTGAGACTGGCCCGA GGTCTTCCGTCGCCGCCCCTGCTGTCGGATGACATGCACATTTCTGCACCTGCAGGACCTCAAAGCGAAGTAACCCT GGACACACCCTTCACTGGGTCTCTTTCCCCCACATCTCCCCCAACAATGGAAGGCAAGCACCGCTCCACACGAAAAT AACAACACGGGTGCAGTTCTGTGCCTCTCCTTAGCTTCTCCCTGCCGCCCCGTGGGCTTGGTCATGGTGTCACCACG CACTGTAGATGTCGCAGACGCAGGCCCATGGCCGTGTGTGCCCCTGGGCATGTCTGCTGTCTTCTGGGGAAATGGAA ACGCCACCATAGGCATTTACTCAGGCAGAAGCTCCAGAACTTTAGATCCCAGCTTTGCCGTTTTTAATTGTCTCTTG TTAAAATGTGAGTATCCCAGATGGAATGCAAAAGAGGTTCGTGCTGCTGAGGCCCTGGTTTCTTTTCTGAATGGATG TGCCAGGGTATTTCCGCCCACAGTACACTTTAAGCCAAAGTAATTTGTATCATCTTTAACACAAACCCAAAACACGA AAGGCTGTGTTTGTTTAACTAGACGTGTAAGCTATAGTTATCTTTATTTCCGCTTTAACTGCATAAGAGAGGAAACC TTTTTAACTATAAGGAAGGTGTTCGGCTTCCTTTTATCTCTTGCCTCATTATGTACTTTTGACTTTATTCAGATTTG TACAAGCTTGGTTTTTACCTCACAGGAAAAGCGAGAACTAATCGCTCCAGAGATCATCTGAGCTCTTTGTGTGAAGG TGTTTTAAGAAGTTGTGACTACATCTTCTTTTCTAAAACAGCCTCCAATTCTGTGATACAAAGTGAAATGCCAGTTG TTTGCAGCTCCGGGGCTGTGGTCCTCCTGGACACCAGCGGTAGCAACAGGCCCCATCCACACAGGGCTGGGGAGACA GGTCCCCTTCTGCACGGGGCCCAGGAGACAGGCCCTCATTCATACAAGGCCAGGGAGACCAGCAGAATCAGCACGGG GCCAGGGCAACAGCCCCATCCACACAGGGCCGGGCAGACAGGCTGAATCCGCACAGAGCTGGGGTTGGAGCTGAGAG TCATCCCTGACCCCAGCTTCATGGTCCTCCAGCTCTTCCCTGCACCATTGCCATGTTCTTACTGCTGCAGCCCTGCG TCAGGGCAGCTGCGGTGCCTTCTGCCTCTTGCCAGGGGCCCCCAGCCCCCACCAGTCACACTGTGGGCCGCACTCAT AGGACAAAGTGCCCTTCCCGCCCTGACACGCACAGTCCTTGCCCACCTTTCTGTGAGTCTCAGTCCCCGAGGCCTCC ACTCTGCGTCTACGTTCACACTGTCTGTCCTACGTCCGGGATGGCTCTGTTGCCTCCTCTCCATGGACTGGCCGTGC ATACCTTGTGTGGACAGAGTTGGGACGGTGCTCCCGGCTGCATCGGCCGCCTGTGCTGGGCCACATGTCCAGGCAAG TGAAGCCTTCACTGGGACCCTCCCAGAGCAGCATGGGTGTGTCGAGGAGAAACATTCTGTAGATCTCAGCGCACGCC CCCCAAGGCAGCCCAGGAAGGAAGGGTGAAGGGCCCGCGGGAGCTGTGCGTCCACAGCATCCACAGTGCAGTGACGT TTCCCACCTTTTCACAGAAGCCTGAGGAGCAGCACTGTCTGCAGAAGGAAACTAGAAAACTTCCGTGCTCCAAGCCT GGGAAGTCCCGGGGAAGCATGCCGTTTGTCTGGGGCCTCGAGAGGCAGGGAAGGAAGGAAGGGAGTCACCTGAAGAA ATCGGACCCAGGGCCAGGGCTTCCCACGGGGGTCCACAGCACTTGAAATCACAACGGGATGTGTCATGCACTCCACA GCCCCAGGTGGCAGCGGGAGACCTCCCCTTCAGGGCCATGTCCTCAGTCCGTGGAACACAGCCCCAGAAAATCCACC CCTGCTGGAGATGGGGAATCCTTTCCATGAGGGCCCACCAGGTCGAAGCCACAGATGAATGAACACCCCAAACAGAA GCAGCCACAAAACAGTCTGAAGCGACTCCCAGAGGTACAGGTTCAAACTGCAGGAAATCAAAGGGAGGAGAGTGTCC AGAAAGAACCAAGAGGGCCGCGCAGTAGCTCACACCTTTAATCCCAGCAGTTTGGGGGGCCCACGCGGGCGGATCAC CTGAGGTCAGGAGTTCGAGACCAGCCTGGCCAACGTGATGAAGCCAACATGGAGAAACCCCATCTCTACTAAAGATA CAAAAAGTTAGCTGGCCGTGGTGGCAGTCGCCTGTAATCCCAGCTACTAGGGAAGATGAGGCAGAAGAATCACTTGA ACCCGGGATATGGAGGTTGCAGTGAGCCAAGATCGAGCCATTGTACTCCAGCCACGGCAACAAGAGCAAAACTCCGT CTCAAAACAAACAAACAAACACCAGGAAGGTTTGAGAAAGAACCAAGTAGACACAGGCACACGAGGCACAGGGCTGC CCCCGAGAGGACAGACTCACCGATGAAGGAAACACAGAGTAGGCCCTCTGGAGCGTGGGAATCAGGGGACTGCAGGT AAACCCTGAGGAATCCCCTGAACACAGCACACGGCCATGGAGGAGGCAGAACGCGTGGATGTGGCCTTGCGTTGTTG GACTCGGGGTGGGGTGGGCCGTGTGGTGCAGTCTAATGACTGTGTCGATATTTCTGGGAACTGTTTTCAATAGTGAG TGCTTGATGATTTAGGTGCAGCCGTGCCATCTGTAAGTCTTACAAAGGGACACTGTCACTTGGGTTGCCTTGCGCTG TGATGCAGACTCTCAGAACACACGTCCTGGGAGGCCTGAAGGTGGAGAGTGGTTGCGGGGCCTCGGTGCGTCCTTGC CTCACCAGTCCCCCTTTCTGCTGCACAGCTAGTTGAGACGTGTGTGCCCATGAAGCAGCCACAGGTGTTTGCCCGTT TAGATGCAGTAGGTGTTTGCTTTTCCGATGTGTGGAGGCAGGGCCCAGAGGTTTACTAAGCCCCTGTTCTCACTTTT GCAGTGCCGTGGAGGTCCCAGCCCCCGCTGCGGTTCTCGGAGGTCCGGAGCCCCTGATGCAGTGCACAGCCTGGCTG AATGCCTATTTCCACCAGCCCGAGGCTATCGAAGAGTTCCCCGTGCCGGCTCTTCACCATCCCGTTTTCCAGCAAGG TCGGTAACTAAGCCATCTGCGGTGTTTCCTTTGGGGAGCTTGACTTATTAACGATCGCTGACATCACAGTTCATTTT ATTGAGTATACCAACTTGGTATTTTTACATCAGCTAAACAGAGGCAGCGTTTGGCCGAGCTGGAGGGACGGGGGTTC GCCGCCTCCACCCACTCCAACACTTGTGTTAGTTTTGGCACAGGGTTGCATAATTCTTGGCAAAGGCAATAAAAATG AAGCATGCAGATCGACTCTTGACTCTCGCTGGCTGAAAGGTTTAAATAGGAACACATCTGAATGGCAGTAAATTTTT CAATAAGCTTATTGTGAACTTCAACTATCTGCTTGCCAACAGCACAGAAAACGAGACTTGTGCGTGGACATTTAGCT ATTTCCATACACTCTGTTGGTGACACTTACATCATAGCAGGTAGCAGCATTGGAGCTTCTCAAATAATTTTACATTT AATTTCTCAAACGTACCAGAGTTAGACATATTCTTTCTTAAGAGATGACTTGAGAACTTACCTGTATGTAGCCTGCT TAAGGCTTGTGATAATCTAAAACATTTCCCAGACATTTCTTTTAAAGCTTGTGTTTGGCTCTGCTCAATACAGAAAT TAATTACAGCAACTAAAATCATTCCAGACACATGCCAGAAATTTATTTTGCATAGTGAGTTTTTCATAATCATTTTT GTGTATAAATGAACATATATTCATATATGATGTATATATGGAGTTGTACACTTATATATCCTTAAAGGGAAAAATTA CCTTGAGATTTTAGATGTGGTGAATTTAAAGCAATTTTGAATTTCAGATCAGATTTGCTGGGTAAGCTTAACAGTAT TCTAATTGCTGCGGTGCTTCTCAGCACAAAACAAACTTGAAAATGGAGAGAACTTTTCAAGGGAAGCAGGTAAAGAA ACCCCTGAAGGTTGGGCTAATACCTGAGTTCACTAACCAAGTCAGACAGGCGAACATATGAAGTCATGAGCACGTTT TGTGCATAATGAACTGAAGCTGTGCAGCTATAATGAACCAGGCACCTTGCTGTCCTCTGAATCATACAGAACTGTCT CCAGAAGGGTTCAGTGGGGATCTCTAGGATGGGCAGATAGGAAGCTAGAGTGGGTGGGCCTTTACATGGAACCACAA ACGTCCAAGAAACCTCTCATCATCTCTGCCCACCAATGGGCTGCTGCCAGGACAAAGGCACTACCAAAGCCTCCTGG GCTGCCGCCAGCTGGGGGCCACCTCTCCCCAGCGCCACAAGCCAGTGAGCCAATTAGACGGTGTGGGGGACCTTGCT CACTGTGCACCTGATGGGCCGGGCACAGGGCAGGATGTTGCTGAATGGCCCAAGGAAGTCAGAGATTGGTTTTGTTT ATTAAAGTTGGGGCTTGGGGATCTTAGAGTGGTGAACTCTTCTCTATACGATCTCTTTTTGGGGGTTCGCCTGCCAG GAGGAGCAAGGCTGAAGAGCTGTCCAGAGGCTTTTCTTCTCCTTAAGCCTGTGAAATATGTGAGCTGAAATGTAAGG TCTCAGGACAGAAAAGTTCTCTTACAAAACCAGGGCAGGAGGTATCTGCATAATCATATGGAGGCTGGGAGAATGAG CCTGCAAACCCCTGAAGTTTACCTAAGGTTTGTTGGGCAACTTCATGAAAAACATAGGGGCAAAAGTTCCATGAAAA TCGTTAGTTTTCTCCAAGTCATATGGTGGCCCTGGCCATGGAGCTAGACCCTGCTGGCCTGGTCTCACCAGGGTCCA GAGAGTGGTGTGCTCTCAGGCAGGCCAGGAAGAACCGGAGGAGGAGGCCTCCGCAGCTGCTGACAGGAGCTCCAGGG CAGGAGTGGCCAGCAAGGAAGGTGGCACCGGAAATAATCAGACACTCTCCAAACCACCCCTGCAAGCACTGAACCCC CGCTGAGCCTTTTCCCCCTCACTTCTGCCCATGGCAGTGTTTGCTGCTCCTAGAGCCGAGGACAGGCGGGCCCCTGG GGCTTCTCAGTCAAGTCCCTAGTGCTTAAGCAGAGTTGGGATTGGCCACACCTACCCCTGCCTCCCTGGCTTTCAGC CCACGCCAGGCCAGGCTGCTTCCTGGGCTGGCAGGTCATGATAGGTCTTGGTGACCAGGAAGACCTCAGAGTAGCCG CCAGGATACTGGTAAAGGCTGTCTGGCTCCCCCGTGGCCCTGCCCCTCTTGGAAAGCCCTACAGTGCGGGTGGATAC CTCTAGCTGGGACGAGGTCCACTCCAGCCAAGTTTTGGGATGGTAGTGCGACAGCCGCTCACATCCTTCTCTCTTGG TGTTCTAGAACTCTGAGGCTGGCTGCAGACCCAGGGTGCTGAGCTCAGGGCCTTCTCTCGAGGGTAGGGGCTGGAGT CCCCTGGGCCAGGCCTGCCTGCGGATGGTTAGTGGTTGCTGTGGGGGCTGGGATTCCCCACCACAGCATGTGTAAGC CAGGTTTTTAACATTTAGCTCAATCGTTTACTCCTGGGCAGGGGCCAGGTGTATGGATTTTACTGTTTGAAGAAGGG GAGGTGGATGGTGGATGGTTGCTAACAGGGAGTTAATTGGTTCTCCCAGATGTGGATACGCCCCTCTCTGTCTGGCA TGAGTTTGGTTTGCTCTCTGTCCAGGTTGTTATCTTGTAACATCAGTGAAATCAACAGTGAGGGGGCAGGTCTTCTA GAGGAAAAGTCTTTTGACATCTCAGAGCCGCCTGGAAGCTGTGGAGTAACAGAAATCAACCTGCTTTGCAGACTGAG AGGAGCGGGTCCATTGTAACATGGAATGAACATTTCCTTGACCAATTCCTTTTTCTGCACATTTTAATTTCTGTATA TGTTTAACATACTTTTGCATATCATTTTTCACCATAGTCTCACTTCTGAGATTGAGACCAGGTTTATCTATTTCAAC CTGGTTTGTCTATGTCAACATTTTTAAAAATGTGATTGTATTTGAGCTCATCCTTGAGTTCCAGCACATTGACCATT TCTTTCTGAAGGCCTGGCTGGTAAAACTTCTGACTGCGCAGTACCAAGCCCTCATCCCTTCCTCAGGCCTGTCCTGC TGCTGAAAGACCCTCTCTCCTGCACACGGCCTCTTCGCGGTACCACCTCACCTTCCAGGATTCCTCTCTGGAACGAA CATGCTGTTTGTGATTTGCTTTTTAAGGCTAACCACAAAAATATGCATTACGTTGAACCATATGAAATTGCTATTTT TTAGGTTAAAACTTATCAAATACCAGAAATTTCAGATGGTTCAACTTCTTATCTTGGGGAATTTCCAAAGACAGGTT AGTAGCTAGGTAGCTGTTGAAGTATTTCTGCCAAGGGACAGTGAAGCTGCCTTTCCCGACCGGGTGTCCGGAGGTCC CCGGGGCTGCCTCATTGTGAGGTTGGGACAGGTGGGATTTCCCAGTGGGAGGGCGCTGAGGCTCTGGAAGGAAGCCT CATTGTTCTGCTGAAGGGATTAAGAGGAAAGCTGAGCTGTCCAGTGAAGCATATTCTAATTTCTCGTTCAAGTGTTG TTTTAGTCAGGTCATTTCCTAAAATAAAATTAGTCAAAGTTTTCTTGGTTTGGGGTTTTTTGGCTTTTTTGGTTTGC TATTCTGGTTTTTTTAAAGTGTCAACAAAATCCATTTTCTAGCTACTGTGTGACCGAGGGCTAGCATCTTGTCGTAC AACGTAGCTTTTTAAGGAGGTAATTTTTTCCCATAAGCCTCTCGCAAGGGTCAGCTTTGTATTTATTTCTAATTCAT TTTTCCTCAAGAAATACTTATTTAATGAAGCCATTATTTTTGAAATACATTTTTACTTTTTTCTTTTATAATGAAAT ATCATTTAAACTTTGAACACTCCCTGATTTCTTAGTGTTTATTTATGAAAATGTTTAGCTTCTTTATATAATGTGCC TTCTGGAGAAGCTGTAGGGGTCAGCAGCCAATGCAGCTACAAGAAATCCTTTCTACCTTAGTTTGCCAAAAGCAGCT TTTTACAATGAGTTCAAGAAAAGCAGACCTACGGAAAGCGAGGATGGACTTTCAGATACAGAACCCATCTGGGCACG TCCTCAGGTCCCTGAGCCCTCCCACCCATGGAAACCCTGCCAGCAGGTATGGCTGACCCCAGGCTGCTGAGGGAGGT CCTGCCAAGGCAGGACCAGGCCTTGGGGCTGCCCACGCGAGACTGTGGGGAGCCTAGCTGGGACCCCAGCCTTCTCA CAGTTGAAGGTGTTCAACGGACGAGTTCTGAGTTTTCTTTGTAAAGTGACTGCAATGTTCCCTTTTAAATGAAATAT TTAACACTTTAATAATTCCTTCGTCACAGCTATTGGCTTACAGCTATGAAATCCAGTTGCATGACAGCAGTAAAATC TCCCTTTTTTAACACCTGCTGGTAGAAGTAAGTTTTTGTAACTTCTATTATAGAGCCTCAGAAAGGATTCATATTTT TGTATTTTTGTGTCTCCATCGAATTCCTCATTTCAAAAGCGCCTCTAACAAAAGCCCTGCCCCAGAGTTAAGGGAAA GGGCCTTCAGGGTGAATTCGTTAAGGTTCCCACCGTGTCCTGGAAGATGGGCCCTCTTTGCTGGTGCTTCTAGTGAG GACTGCTTGGGTCCTGGAAGGGGCATGTCCCGGGAGAGGATGACGAGGGAGGAGAGGAGAAGAGAGTGGCGTCATTA CTTGGGTCCTCACCGGAGCCAGCCCTTTAAGATTAGAAATCCTTCTTGAAGAGCCAAGCAAAGGCTCATCCAGCCCC CTACAACCCCATCCTGCTGCCTACTCACATCCAGGAAAGAGCCCACCCCTGATGGGTGCCCAAGTAGATGGCCTCCC CTGGAAAGAGGCATATTAGCCCTCCTCACCCGAGGGTCCTAGGGCCCGTTTGGGTGTTCTTAAAATGTGCCTTATAA GTCGTGATCCAGCCTGAGTTTTTCTACAGAGACACTGCAGCCAAGAGGGGATGTTTTTCTTGTAGCTTCCACATAGA TCACGGCAGCACGCAGGCTTTGTCCTTTCAGCACTTTGTACCGATATTTTCCCTGAACTTAAGAAAAAAATTACCAA TTGAAAGAGAAGAGAATAGCTAAAATAGGAGATGCATTTGCCACCACGTGTGGGTGGCCACCGCTGTTCCCCTTGAT TTTAAACGAGGGCTCATAAATATTGTTGACCTTGCTGAGATATTACCTGGTGGAGCTGGAAGGTTTGTAAACCTTCT TCCGATGCCCGTTTATTGATTAGAACAAGCTAACAACACTGCACTTAAGTGGAAAAAACAGCTGGTAAAAATGTAAT TCTTTCAGCCTTAGGGCTGCTGTGCTCCTTTTCTGGAGAGCAAATGTGGAAATCAAGATCTGTGGCCTTCTGGAACC TTCACCAGGAAAGGTGAAAAGAAGAAGAACCCATCCACAGCTGAGAGTGGATGGCAGGCCCGCGGGTTTCCAGGTGG CCAGTAGGATTGTCCGATGTCTGGAATGAGTACCGGGGCCGCCACCAGGAACACTTGTCATTTCTACCATGGAGAGG AAAGCCCAGTGCATTTCTGTGGTTCAGAAACTTGACACAAAGAGAAAAGCTGGAATGTTTAAAAGCAAAAATAGGGC AGAGATCAGTGAAAGTGCAGGATGTGTGTGAAACAAGGGTAGAGTGAGCACTGGTGGGGATGGAGGTGGTGAGGGGT CTTCTGAGCTGGGGACGTTTACTTACACCAGCCCTGCCTCCCAAGAACCCATCCCCTGTGGGTCCAGAAGACCAGAT CCCCGCCAATGTCCTTCCCTTTCAGAGCTGTCTGGAGGAGCATGCCACTAGTTCCCAGGGTCTCATCTGCCGGCACA CGGCCTGACTTTCATGGGAAGTCCCCATTGATGCTGTTCCCTTCCCAGTGAGTGGGCTCCCGTCACCCGTCCTGGCT CATTTGAATACCTGCATCACCTCCAGCCCCTCAGCAGAACAAGAATGTCAGATAATCCAGAATGCTGTTGAGAGCTG CTCCAGGTTGGGGGCTCGCAGCCCCAGCATCCAAAAGCCTGGTTTCAGCAGGGCATGCCCGCAGAGCCCCTAGGCGC CCCGAGCTCTCCTGGTTTGCCCGGGCTTTGCCTGCACGGGCTGGCCCCGTGGACTGCCTGGCCAGGCCAGCCCAGTA ACAGCGAAGCACACCACTCTCAGCTCCTGAGCTTGGCTGACCAGCTATACTCTGGCATATTGGTGAATTTCATGGGC CAAAGAACCTGAGGTTCAGATTGTGTCTTAGTTCATTGTTTCATATTTGAATATGCAGTTTCCTACCTAGCAATTGT CATGAGTACTTTCTTGCTGTTTATACTATGAACAACATCTTGGGCTAGGAGAGAGTTTTTAAAGTTTCAACAATGAT TCATTCAAGCACTTTCCTTTTTCAAGTTACTCATATCTCAGTTCATTCAATGGGGTTGGGGGATTTATATCAGAATC CTCTAGACATTTCATTTATAATATTCTACCTATAAATTTTTATACTTCTTACCCCTCCTCAAGATACACTAAAACCT TAGCATGGGCACACGCACACACATACCCTGTTTCTGAGAATTATTGTTCTAAAGGAATTTATCAAAATGGATGTAGC TCCTATGAATGCTTTTGCTGTGTGCTTTACCCTCTACTAAGCCTAACATTCCTAATTTAATAGCTATTTTTGTTTGA GACTTAAAAACTGTGCGCATTGAAGGAATGTTTTGCCGTTGAGGAAATGCCAGCCCCTCTCCTAGGAAAGGGGTGCT CGGCTCCTGGTCCACTGGCAGTGGCTGGAACAGAACCACAGCCTGCTGAATCCCAGGAAATTCATGATGTCCCTGTG CAGAGCCCTCTGACTAATCAGCGATCCCACAATGCCTGTAGCAGAAGCATAGTTAGGTGAGAGTTTTATCCTGAATG TAAGAGGTGGCCAGGTCCTTAGTTCCCTGAGGTTCCGTGAGTCGGGGGTAAAGGAAGCAGCCCTCACACAGAGTAAT TTACCAAGCCTGAGAGGGAACCAAGAGGACTGTTTGAGAGTCAGCCACTTCCAAACCTTGGTGGACATAACGAGGGT CTCTACTACAATAAGCAGGACTTGTAATATGTAAAATTATTATGAGTGCTGCTTTTATGGGAAAATATTCACATTAA AATATAATTTAACTCTGGTGAAGTAATTAAACAGTAAATCAGTGATGGCTTCTATTAAAAATTAAGTCATCGTAATT ATGTGAATGCCTTTAGAAGGATTTTCCGCAGTGTCCTGGCGTTGGCCTTGAGTGGACTTCTCAAAAAGTAGAAGTAA CCAGGTTTCTGCCTGTGAGGAAATGTAAATTGCATCATTGCTTCCTTTAATAAATTTTCATTTTAAATGGGCTTTGA AAAATAGGAATTTTCTCTCTGTTTTAAAGTAAAAAACACACTTTTCACTTAAATATGTTTCTAAATTTCTATTTCCA CTGTAGTCATTAGCTGGACATGTGCCGCTCCAAAAGAGAATGTTAACCTTCGTCTCTTTTGTCGTCATAAATGCACA TTCATGTTTTACAGGTAATGTTCGTGCATTTGAAATAATTCCAGCAATAATAAGTAGAATTATCTCCACTACCTGTG TTTATAGAGAAAATGATGTTTTAATAACGTGTGCATAGAATTGGACTATGTTATCAATACGTTAAATGTGTAAATAT TAGTATAAAATTAATAGTGATAATGCTGAAAAACTATTGCAGGAGCTGAAAGTGTAAGAACTATGACTTAATGTAAA TGCTTTCACACATACGGTTTTTGCCTGCAATCCTTATGTTCAGGCTGACTCGGAATATAAGATTCATGAGTCTCTGT CCCTCTTTATATGTTGTTTCATGTGTCAGAATGGAAAAAGCAAATATTTACGTAGAGGGAACTCCTCCCTCCTGTTG GAGGTACGCGGCGGTCAGTGTAGCTTCTGAGGCCGCGTGGAGATGGGAACATCCTCAAGACAGAGCAGACTTCCTGG AGGCTCAGCCGGAACGTCTCCAGCTCTGTAGCACGTTTTAATTCCATACAAGAAGTTACTCCCAAAGAAGTAAATTG AAGAACGAAATCGAGGAAAAGAAAGTCCTTTTTTGAAAGCTCTTAAACACTTGACTCCGTTTAGTTCGGATCAGGCC TTCTCTGCCCTTCCAAAGATTCAGGTTAGGTATTGGTAACGCCCAGGAAGCACCCACTCTGGCGAGCCCACCCTCTC CGCCAGTAAATATATACAGTGTAGCTGTTACTATGACAACGACTTTTTTTTCTTGAGTACTGTTTTTAATTGTTGCA CAAACACATATTGTGTAAGAAAGGGGAATTGTCCATAGTCTTCCACGTGTGGATGTGTGTGCCCACACTGTCTGCTT TCTTTAAAAATGCACATTCCTTTCTGGATTGGGCTCCGCGATCCCCCGCCTGCCAAGCAACCTGATTTTCTCTTCAG CCTTTCCTCACTTAGATGGTTTTAGTAAGACCTAGCAACCGAGCTCAAGAGACACGTCAGGACTTACTGCTTGCCCA AGACATATACTTTTCATAGAAAATTCGCGTCTCTTCCTCTTTTCAGAATAAAATATGCAAGAACCAACAGAAATGGC ACATTCCCCTGCTTTTCCCCAAGAATTGGGCCTTTGATGTTCAAGAGCAGCTGATATTAAAGGCTATCCCTAGAAGG CTGTAGAAACTGTACCTATTTATGACAAATGTAAAACAGCACAAATCTAGCTGAATGTGATGGTATCATTAGTTTGT GAATCGCATCCTTGAAAATAACTGTGAAGGAGCCAAATCGGTTTAAAAGAAGATAACAAAATGGTTTAAGCTACTTA GGTTGAATGCCAATTATATTTTAATATTCAACTGAGCATATTTTACTAAGAAAAAAGGCCGCATTGACGCATGCATA ACTCATTGGCAGCGTCGGCCAGCTGTGCCGCTCCACTCTCCGAATCACTTTGTGCATTTTAGTAGCATGGTAATTTA GTAGAGGACTACCTGAGTGATGGCATTTTCAATGAGCAGGTAATCTACAGAGAATTTTTATATGGTTATGAGTTCTG TGCTTCAGCCCGGCAACACAAAAATACATCAAGACCAATTACGGTATCAACTTTCAGTGTGATACCCTTGCCACGCA AGTGCATGTATTTCTGCCCCAGGCATTTCAAATTGCATCTAATTTGAAATCTTTGGGGTTTTAATTAAATTTCAGAA TATATGGAATTCTTCATTAGTACCTGCTCTGAATAGCAGATCATCCTTCTCCCAGTCTCTCGTTAAAGAAGTAGGCT GTGGTAAATAGCGTGTTCAGTAGCTGATCATTTGTGGGACCTGGGTATTGGAACAGAAATAGTGAAATGCAAAATAA TATCAAAAAGATTATTAGTCCAGGGAGAGTCTCTTGATTTGTATTTTAATGGAATTTTACATTTCTCTTAATGATTT GCAAATTGTCCTCTGATTATAAGATGCATTAAGCTTTTCATTTTAATGGCAAAGACACAGCCATTAGCAGAATTTTT TTTTCTGTCTTCTTTTTAGAGTGCTTCCCTGCAGCAGATAAATATTGAAACCATTAACACCTTTTGATGTATGGTAC AGTCTGCATCTTAGACCCACACATGGCTGGCCGGCTGTGGTCCCTCCGTGCCGCACACCTGGCCGGGGACCCTGACT CTTCTCTGAGGACAGTGAGCAGCAGTAATGCCTGTTTCCTTTTCTGGAAAACAGGATTCTTTTTGAAAATTTCATCA GAAGGTGGTGTTTATTTGTATTAAAGCAATGCATTTAAATATTATTCATGGCCCAATGTGTATTTAAAAAGCTGGTC TTACCAGTGTTAGGGTTGACGGTGCCCACAGCTCCTGCCTTGCTCTGGTCTGCGGGGCTGGCCTTGGTGATTTTGGA AATGGAATCTGGCCTTGCCCCCAGGATGTTTCTCTGTCAACCCATGTGCTGTGAGAGACACACAGAGCTCTTTCTGT GTTAGCCAGCTGACCACAGCACGACCCCAACGTGGGGACACTTGACTTTGGTTTGGCCTCTGAGATAATGTCTTGGT TTTAAGGCCAGGACTTCTAATGCCCTAGAAGATGAATATGATTTACACGACCAGAGCTACAAGGCAAAGCTCATCTC TTCTGCTTCATCTGCTGAATGTGCACAGGGAGCTTTAGGCTTCGACCTGGCATGCAGTGACAGTCTGTGAAGGCAGT GGCGCTTCGGGCTAGGTGTCTCCTTGTGTCGGAGACCTGTGGAATCCAGCAGCTCCGTCTGATCGTATAGTTAGGAA AACCAAGGGGACTAGAGAATGGCACTGCTGAGTGCGGGCAGTGGTGGCTGCCCCCCACCCCCACCCTGGGCAAGTGA GGTCAGGCCTGGCCCTGCTTGCCCTCCATTTCGAAGGGCAATTCTTCACTCCAGGCTTGCACAGCTGACCCGAGAGT GAGTGTCTCTGAGCAGCTGTGGCTCTCAGTCACTTACCCCCTCACCTTCTCACTTCTCTTCATGGGTCTTCCAGGAT CTGAGGGATGAAGCCAGACACACCTCCTGCCCTTCCTTTGTCCTCTCCTGGTGAGTGTCACCTTCCCGGGCTGTCAA GAGCTGGACCATGTGCCCACTCAGGTGTGTCACCTTCCTGGGCTGTCTAGAGCCAGTCTGCATGCCTGCTAAGGCGT GTCACCTTCCGCTCAGGTGTGTCACCTTTCTGAGATGTCTAGAGCCAGTCCACGTGCCTGCTCAGGCACATCACCTT CCTAGGCTGTCTAGAGGCAGTCTGTATGCTTGCTCAGGCATGTCACCTTCTGCTCAGGCATGTCACCTTCCCGGGAT GTCTAGAGCTAGTTCGTTTGCCCGCTCAGGTGCATCACCTTCCGCTCAGGTGTGTCCCCTTTCTGAGATGTCTAGAG CCGGTCCATGTGCCCGCTCAGGTATGTCACCTTCCTGAGATGTCTAGAGCCGGTCCGTCTGCCTGCTCAGGCGTGTC ACCGTCCCAGGCTGTCCAGAGCCGATCTGTGTGCCCATTCAGCTGTGTCACCTTCTGCTCAGGCATGTCACCTTCCC GAGGTGCCCAGAGCTGGTCTGTGTGCCCGCTCAGGCGTGTCACCTTCTGCTCAGGCATGTCACCTTCCCGGGCTGTC TAGAGCTGGTCCGTGTGTCCACTCAGGTACGTCACCTTCCCAGGCTGTCTAGAGCCAGTCCGTGTGCCTGCTCAGAT ATACTGTCACCATGTGGGCCCTACTCTTGTGTCAGGGCCATTAGCTGGCACATGCTGTGTGGTTGCCTGGTGCGCGC TGGGGCAACCTCGGGAAACACTCTTAGCCTTTGTATTGATCTGTGCAGGCTGCCAAAACAAAGTGATGCCAACTGGG GTCTTATACAACAGAAATCTTTCTCAGCTCTGGAGCCTGGAAGTCCGAGACCGAGGTGCGGCAGGGTGTCAGCTACT CAGACCAAGGTGCTGAGGCTGTCCTGCTTGCCCTGCAGATGGCCGTCTCCTTGCTGCGTCCTCACTAGGCCTTTCCT CTGGGCGTGCATCGCTCGGGTCTCTATATCATAATCTCCCCTTCTTAGAAGGACACCAGTCAGATGGGATTAGGGCT ACCTGAACAACCGAATGAGAACTTCTTCACCTCTTAAAGACCCCATCTGCTGATACAGTTGAAATGCCGAGGGTCAG GGCTGCAACATACGAATTTTGGCAGAACATAGTTTAGCCCATAACACCCTTTCCGAGCCTCTGATTATCTGTCTGTA AAGTGGGGACAGTAGTAACAATGTGTAAAATGTCATGAGGGTAAAATGAGGCCACCAAACAGTCAGCAAAAGAAATA CCTGCTTATTTCACAGATAGTCTTTTCTCCAAAATCCCAGTGGTAGTTAGCAAGTCAGGACAGCTGACTCCACCTGG TCCTTTGGAACTCAGGGAAGATTGGGCCGTTATTAGGAAGTGCTGCTCTTTGCAGCCAACAGAACTTCCCAAGGACG CCCGCCCTCCGCCCTTGCTTGCTATGTGTACGAGTTACAGGAAGTGTGGCTGGTATAGCCCCGCAGCTGTTCAGAAG GAGTGTTTTGGTTTGTCATCTCCACATGAAATGAATGGAGCCAATTCATACTGTAACTGAGAAAAGAACATTTTTCA ACCATTATTTCTTCTCCCTACTCACTGTTAAGCTCTAGGCCACAATAAATGGCTGCCTGCTGGGACTCCTGCACTCT GGGATGACACGTTCAAAGCGTGTGCCCATGTAGGCTGCTGCAAGAGGGTAAGCATGATTGGAGTGGGGTCACTGGGG TCTGGGGAGCCAGCCTCGGCCCCTCCTGGCTCAGCACAGCCCTCGCGGCCTTGGGGGCCCACAGGGTCACTCTCCCC CAACCCTCCCACAGCACCGGGCTGTCTGCTCTGTGCTTCACTCTGCACTAGCTGGTTCCTGAGTACCAGGAGGCATT CTCTGATATGCTATGGATACAGTTATGGACAAAATGGACTCTTAGGAAGGCAGCAAGTAAGGGGAATGGAGGGGCAA AGAGCACACAGCTAAAGATTGCCTGACTCAACTGTGGTAGTCACTGGTGCACTGCAGGTTGGCATTTGCACCCTGCT AGCATGTGCTCCTGAAACTGGGGCCGGAGAGCTTTCTTCTCTGTCTGAGCACTCAGAGCAGTGCCTGCCAATGCTGT GACAATGAACGAATGAATAAGCAAAATGTCAACCCATTTTCAGAGTTTTGTTTAATGTCCCTGGACCTGGATCTCTC TTGAGAGTGAGCATTGAATGGGCCTTCCCTGCCCCTACTGAGAGCTCAGGGCATGGACCCCAAACGCTTGGGTCTCT CCTCTTCCCTCTTCTGTTGTAAATTTAAATGTCAGGGGGAAAAAAAAAAAAGCAAGAAAAATATGTAAATAAATACA TTTGCAATCATATGTTATCGGATGAGAAGGGTAGCGGTTTCAAGAGTTACATTCAGAGTAATCGTTGAGCACATTGA TGGCAAGTTGATAGTTTCGTTATTATGAAATATTTACTTTGGACAAAGGAGTATTCAAAACTGACTTACGAGGTATA AATAATAAAGGACAAAGTCATATATATGCCACCCAGGTGATGTAATAATTTGTTTTCTTCCCTTCTGTCAGTAGACA TTTGGGTTCTGAAGTTTTTGCCTTTAAAACTAATGATGTTACAGATATTCTAGGTTGTGTGCCCTAGTGCTCCAGCT GGCTGGAATTCATGGGCGGAGTTCGCGAGAAGAGGGAACTATACAGAGAAAGAATTCCAGAGGTCTGCATGGTATCC CATTGAGTCTGTTGCAGATTACAAAGTCATACATGCATTTGTAAACCACATGAAACCAGACAACTATTGGGGAACTA TAAGTTGAACAAATTTCAGATAAGTGAACCTCACTAGAACTAGAAAATATGTGGACAAGTATGAAAATTTTCTTCTA TTATTTTATTTAAAAGATGATTAACTGTTCATGATAAAGTTAATAAAGATGTATTTTGTAGTTTATAATACACGTAG AAGGAAAACATATGACAACAATAGCATAGAAGATGAGACTGGGGAAATGGAAATACATTGTTCTAAGTTTCTTACTT TATGAACAAGTATAATATTTAAGGGCAGACTTTGATAAATTATGAATGCATATTAAAAATCCTAGAATACCAGTAAA AATAACCAGAATCTATAGCTCATGGGCCAACAGAAAAGATAAAATAGAATGCTAATTCTTTTATTAGAATTAGCATA TTAGAATGCTAAAATGTAAAAGAAGATAGGGTAAAGGGGGAACAAAGAACAGATGGGAAAAATAACAAGTTGGCAGA CTTAAACCCAATCATATTAATAATTACATTAAATGTAAATGTTTTAAGATTCAAATAAAAAGACATAGATTAGTAGA CTGGTTTAAAAAGCCAGAGCCTGTTATATATTTTCTACTAGACAAAAATAATGTAAAATGATGACAGAGTCCTATGT AAAGATGGAAGGAAAAAAAAAGGAATACATTGACAGAAAGATATAAGTAAAATTGTATTTATTTTCAGATCACATAA TTATTTACATAGAATATCTTGAATAATGTTGAAAATAACTATTAAAACCAATAAGTGAATTTGAGAAGTTTGAGGAT ACAATTTTAACATAAAATGTATTCTATTTTTATATAATGTAATTAAAAAGTAAATTATAAAACAGTTGCTTTATGTA TTTAACATTAGCATTATTAAAGATTATTATTTAAAGATATGCAAGGCCTCTCTACTAAACAGCATAAAAACATGACT GAAAGAAATTAAAGAAGACCTAAGTAAATGGATCTTTATTAGTTATTGGAAGAGGCGGTGTTACGGAGAGGTCACAT CTACACAAATTGCCCTCTGCATTCAGTTCAGTCACACTTTTAAATTATTTCAAAATGCAAAGAGCCTAGAATATTTA AAACAATCTTGAAAAAGAAGAATGAATTTGTAGGACTTACATTACCTGGTTTCAAGACTTCCTACAAATCTGTGGTA ATCAAGACACTGCAGTAAGGATCAAAACTTAGAACAGTGCAACAGAGAGAGAGACCAGTGTTTTTTCAGTTCTTGTA TCTTTGAGAAAGACACAAAGTAGATCAATGAGGAAAGTAAAGTCTTGTTGGCCAGGCATGGTGGCTTACGCCTGTAA CCCCAGCACTTTGTGAGGCCATGGCAGGCGGATCACTTGAGGCCAGGAGTTCAAGACCAGCCTTGCAAACATAGCAA GACCCTGTCTCTACTAAAAACACAAAAAACAGCCAAGCATGGTGGTGCACACCTGTAATCCCAGCTACTCGGGAGGC TAAGGCATATGAATCACTTGAACCCAGGAGACAGAGGTTGCAGTGAGCTGAGATCATGCCACTGCATTCCAGTTTGG GTGACAGAGGAAGACTCTATCACAAAAAAAAAAAAAAAAAAAAAAAAAAGTCTTGTCAACTAATGGTGCTGAGCAAC TGGATATCCATTTTTTTTTCCATTCCATTTTCAGATCACTTTAATGGTTAGAAATTTGTTCCATAACTTATTTATTT CCATAGGTTTTTGGGGAACAGATGGTATTTGGTTACATGAGTAAGTTCTTTAGTGGTGGTTTGTGAGATTTTGTGCA CCCATTACACGAGCAGTATACACTGAACGTGATATGTAGTCTTTCATCCCTCACCCACTTCCCAACCATTCCCCCTG AGTCCCCAAAGTCTATTGTGTCATTCTTATGTCTTTACCTCCTCATAGCTTAGCTCCCGTGGATAACCATTTTCAGA TAAATATTATTCACCATGACCTTGACCTCACACCATACACAAAAATTAATCTGAACTATATCATATACCTAAAGGAG AAGCTGAAACTCTAAAGCTCTTAGAAGAAAACATGAGTATTTTTCTGATTTGGGTAGGCACACATTTCTTATAACAC CAAAATAAAAAACAATGAAAGAAATTATTAATAAATTAAACTTTATTAAAAGACTTCATTAAGTAAATGAGGAACAG GCCACAGAATGAGAGAAATTTTTACAAAACACATATCTGACAAAGGACTTCTAAGAAGGGTATATGAAGACTCTGAT AACTCCATAAAAGGACCAACAACCCAGTAAATAACTGGACAAAATATCTCACATTTTCATCGCAAAGACACATAATA CACAGATTGGCCCCTGAGCTCATGAGAAAGTGCTCGGCATCATCAGTCATTAGGGAAGTACAGGTTAAAACCACAGT GAAAGACATCTGCATATATCCCAGGGCAGCTCAGATCAGACACCACCAAGTGTCGCAGAGACTGCCGCAGCCTCGCC TCTCGTATACATTGGTGGGTGTTAAAATGTTGTAACCTCTTTGCAAAAGGTCTAGCAGCTGCTTATAAAACTAAGTG TTCTCCTACCCCGCTACCCAACAATTCCACTCTAGGGCATTTATCCAGGAGAAATTAAAACCTACGTCCACAAAAAC GTATTCAAGAGTGTTCACAGCAGCTTTGCTTGCTTTATTTATAATTGCTGCAATTGGGAATTGCTTGTATGTCCATC TGTAGGAAAGTGGATAAATTACCTGAGGGATATCCATGTAGAGCCTGTTCCTATCTGTGAACTTAGCATTGAAAAGG AATAATCCACAGACACACACAGCCTCGTGGATGAGTCTCGGTAACGTCATGCTGAGCTTAAGGAGCCGGACACAGCA AGAGCCTGCCTCTGATTATATTTATTGAAGCTCTGCAGTTGCCAAAAACAATCTGGATAGGGAAAAGTTAGAAGAGG AACTGCTTCTGGGGTTGTGCAGGCAGTGAGAAACTGGGAAGGGGCATGAGGGAATGTTCTAGACCATGACGGCCATG GTCTATATCTTAACAGGTTAGGTCACATAGGTATCTATCTGTGTTTGTCAGAATTCATCAAATGATGTTGAAGATGG TACATTTCACTGTGTGTGTAAATTCCACCTTAAAGGAAAAAAGAACCATAAATGGACATTGAACTCTTAATGGTATA TGTGCTGAAGGACTTGGAGAGAGTACACTGATGTCTGCAGCTTATTTTGAAATGCAGCAAAAATAATAATAAAATGG ATATGGGGTGGAGAGATGGATAGATATATGATAAAAACAAATACAGGCTAAAATGTCAATTATATCATCTGAGGTAT AAAGCTATTCATTGTAAAACTCTTCCAACTCTATGTGGCTGGGGTTTTTCATAATATGTTGGAGGAAAATAGTAAAT ATTATGGATTTTTATTCCTTGAAAGCAAATTTGGGGGAAAGATCATGAAAACTGTGGCCTCCTTATCCCAGATGGTG AAGATACAACCTAGTATGGCCTCAGCGAGATACTGAGTGCACTCTCAGTTTGCACCTGGAGGGCAGGGTGAGTCTGG GAGGTCTGTGAATTGTCAGTGGACCTCCCGGTCCTCTCCAGAGCCCTGCAGGATCGGGTGGATACACTGACCTGATG GACAGGAAGAGGAAGCCTCTGGGCCAGACCTTTGGACACTGTTGCCTTACTCTTCTTCTCTCCAGCCAAGCGAGTGG CCTTGGGAGGTGAGTGTGGTGTCGCCTTGCAGCCTTGCACTCAGCTTTGCAGCCGTCCCTGTCCAGCCAGCTTCCAC GCAAGTGGAAGACACACAGAATCCACAGCCCAGGCAGCTGCTCTCACCTGTGTGTGGAGCACAGGGTGCCCATCCTC AGAGACCCTGCCCAGCCCCTCGCTGCCATCCCCTCTGCTAGACCAGGCATGTGGGTCAGCGCTGCCCTGTCTGGCCC GAAGTTCCATGTCAGTAGCCGTGGATCAAATGATGAAGGCAGAGTTATCTAATTACGGAGTGTTTTCTCTAATATTT CCACAGCCTGCTGCTGTCATGGAACATGGAGATTCATTACTTAGAATGTCAAATTAATAGCGTTCCTGGACCTGAAA CATCATTGTGTCAACTTCTCCACCTTTTCCAGGTGGTGGTTTGAGTTTGTGCACATGGCATGCCTATTGCCCCAGCA CGGCCACAGCTTTCCAAAGTGCCGCCCAGGGCTGGGGTCTGCCCACTTCCTCGGAGGTCTGTCCATGCTCTGGGCAG GCTACAGCCTGCAGGATGCAGATGCCCAGATTGGAGCAGCACCCTGTGCCCTCAAGAGGAGCACAGCTCACACATCC ACGTAACTCTTGTCCTTAGCAGTAGTGAGGGGCTTCTGCAGGCAGAGGAGTCGGCTCCACGGTCCTCCATGTAAACT ACCGGGATCAGGAGCCTAGGGTTCAATTCTGCTGTCACGTGTCTGCACTGTAGCCTGTTCTGCCTTCTCAACCAGCC CCTTCTAGCTCCTGGGGCAGTTTCTCTTTAGCTGAAACTTCCCTTCATCTTCCACCAATACCATGTTGCGTTACAGT TTCCCTCTTCTCACAGAACGAGCCTTGGGAGAGGAGTGTGTTCTGTCACACAGCCTGGCCATGGCTCAGGTCCACAA GGTCAGCTCCAGACTCTCAGTGCAATGAGGGACCATGAGTAGCTTCTGCTTGCTGAGTGCTTTCCAAGTGGCGGGGC CTCTGCCAAGTGCTTTGTGATCCTCATATCCAAGTGGAGGAAACTCAACTTTAGACAGTGACTGAGCTCCAACAGCT GATCCTTGGCACAGATGAGACTTGAACCTGGGTCCTTCTGACTCCAGAATGCAAGCTCATAGGAAGGGGTAAATTTA CTGGGAAGATCTCTGCTGGCCAGGTCCTTGATGGCACAGGGCCACGTGCAGGTGGAGTCCAGGCGAGACTTGTCCTT GGGAACACGAGCTGGTTTTGAAGCTACTGTAGTCCTGGGTTGCCCGCCCATGGGTGTTGTGAGGACGCTCTCCAGCA TCACTGCTGCCCACGTGCCCTGAGGGGTCTTAGGTTCCACCCCAGGTCACATTCAGTGTTTTGTCTGATTCTTCACC AGAAGGGAGCAGCCTCACGCAGACTGAGCAGGAACAGTGGCATTGGTGTTTTCCACAGATCTTTTGGAAGAATTTGA ACTTCTGATCATGTATATATGACACTTTTTAAAGTTTTAAGTGATGCATGGAAACTTGATATTGTAAATTTAAATAT CAGTCATTTTCAGTTTTGTCTCTTTGCTGCTCAAAATGAAAAATCTTCTCACTCTTCCCCTACTCCATAATATGGTC ATGGAGTTTTGACCTAATTAAAAGCATCAGAATTAAGTTGCTAAGTTTGAATGGGAATGTAGTGGTGTTTAAGGGGA CTTAGGAGACCCTGTCCTCATGTCCTTTCTGTCACTATAGGTTTTGTTACTGTTTAAAGTCCATCTGCCCAGCTTTT AGATAATTAATTCTAATTTTGAGAACTTGGGAGATGTTGCAAAAACTGTTGGAGCTGATTTTAAGATTGGTGTAATA TACCTGGTATTACTAGAGTCAGTCAGTGAAGTCATAGATGAAGTTGTGACATTTGGCCCAGGGTTCCCTGCTGAATT CTGAAATACCATGATGCTATCAAGAGAAGAGCCATGGGTTAGCCAGTCTCCATGTGACACCCAGTGAGCTTCACTGC TGGTACTCCCACTCTTGTTCCTTCTCGGATGGAGTCAGGTCTGGCCCCAAGCTACAGCAGGATATGGAGGAAGTGAT GGTGTGCGACTTCCGAGGCTAGGTCATAAGAGGCCTTGCAGCTCCCAGTGTCCTCAGTCACCTGCTGTGAGGGCAGC CAGTACCATGTCAGAGGACATACAGGCAGCCCCAGGAGAGGCCCATGTGGTGAGGAGCCAGGGAAGTCAGCCCTGTT GGAAATAGATCCTCCAGCCCCAGTCGAGCCTTCGGATGAGTGCAGCTATGCAGAAGACCCCAAGTGACAACTGCTGA GCCCGACCCTTCTGAATTCAAGGCCATGAAAGAAAGGTCATTTGTGTTGTTTGGGGCCACGAAGTTGGGGATTATCT GTTACCTGTCTGTAGTGACTGGCACAAGATGTGAGGCACTCCCCAGTAGTTACTTATTCATCACACTTCCTTTCATA GCTGGTCCTTGTCCTTTCCTCTGGTGTAAAGTACAGACTGTGAAGACCCGCCCTCTGTTCCTTCCAGCTGTTTTTGG CTGTCTCTTGTGTACAAATGCTGTAGGAGATGCAAAGGTGACTCGCAGCATCCCCCAAGAATGCCTGGGCTTGGTGC TGCCAGAAAGGTATATGAGGAGCCCCGGGTAAGCCAGCACTGCAGGCAGCAAGGTAGCCTTCAGCTGTGTGGGCTGG GGAGAACTTCATGGAGGAGGTAGCATTTGCACCAGACTTTGAGGTAGATGGGATTTGAACACATGGAGTAAGGGGAA ACAACATAGACTGAGGCTGGGCCTGTGTGTGAAACAGCAAGCAATCCCTTCTGGCTGGAGCAGGGGAGGGTGTGAGG AGGGGCTGCCAGCCTCCCTCCAGGCCAAGAAGGCCATCAGGTCTCACTGGGGGACAGTGAGGAGCTACTCAAGGCAT GATGGGAGCCAGGCTGGACTTGAACAGGACTCATTGGACTGTCCAGGTGCTGGGTGAGAGAGGGAGGAGGGGACATG GAGCCAGCACCAGGGGACAAGGGGGACCTGAAGCAGCTGTGACCGTGGATGGCAGGGAAAAGAAGAAGAAGGGATTC ATTTTGGCATAGAGTTGGGAATTGCAGCAGTCACTTGAGAATGACAGAGATGGGAAGGTTAGGGATTAGGGTCAGTG GCATGGACGTGAGCGATGAACCCATACAAGTGGGTGAGAAAAAGGCTGAAGAAGGTGAGGCAGCATAGGGAGTGGGT GACGCCAGTGATGGCTGATGTGGTGTGACCAGAGGAAGGGGCCAAGGGGGCGGACCCCGCAGGGTCCATAATGGTGG TGATTGCCTTGGGGGAGTTCAGGGGTCGCAGAAGAGGACCTGATTATGCCATCCTCACTTGTAAATTCTGCTTGGTC TCCTGACGCTATCAGGGTCAGACCCAGCCCTCTCATGGCCCGCTGTGTGGCATGTGCTGCTCCCTGGCTCTTGCCCA TGCCCCTCCTCCTGGCCAAGCCACACTCCACCTGCATCAGCCATAGGCCCTCCCGCCACTGCCCGCCTGCAGGTCTC AGCACCAGCGTTGCCTCCCCAGAAGCCTCCCTGACCATGGTCCCTGTTGTGTGCCCCACCACCCCCTGTTCTCTGTC CCCCTGGTATTGGTTCGTCTGCTTGTGGTAACTGCTGAATGCCAGCCCCAATGCCCCCGCCTCCCATCGCCATCAGC GTCACCAGCATCACAAAGGTCAGCACGAGGTAGCTGCTCACAGAATATTTATTAAGAATAAGAAATTAATAAAGGGA CTAATTGGTAAATCTTAACATGCCCACACTGGGTCTTTTCCCCATGTCACGTGAGCCTGAACTTGGAACATCCATAT TTCCAGAAGACATCAGGCTCTTTCTCTAGTGACCTTAGAAATCCCCGCATTGAAGGGAGATGGCCCTGGCATGGGGC CCCTGCCACTGGGTGGGGAGGGGTGGGGACCAGACTGTGCCAGGGACCCCAAGGAGTTGGAGGGTGCAGAGCTTCTT GACCTGAGTGGACAGGGAGACCCTCTGAGCTCATCCTCTGTAACAGGATGAGCATGGACTCTTCAGGGGACAAACTT CGTGCCTCGTGTGAAAAACGAAAGGGCCCCACGGGCTGTGTCTGATGGTGTTACCCACTGCACAGGGAAGGCACCTT TGCACCCCAGTCCCAGCCAGGAGAGCAGCAGGTCGCAATGTCTTGCTAGCCTGGCCCCTCTGCCCTCAATGACTTTG CAAGCACCTTGGCCTCCCTGTCCCACTCCCCTGCCGGCACCTGCGTGTCCCTGTCCTGCCGAGCTGCCAGCCTCACC TGCGTGCCTCTCGTCCTCATTTGAATAGGTGAGCAACCAAAGTCAGCTGTGCCGTTGGAGCCGGATTCCATCACTCA AAATCCAGGCACAGCTCCTTTTGAATTCCTCAGCTGCCCCGAGGCAGGGGCTCAATGTTGATACTGAGATTAATCCC CAAAGAGCTTCTCATTTCTCTGCAGAGAAATTAAAGCTTGCCAGTTTCCTTTTGCCCGTCCTTCTGACAGCTCTGTC AGTAATGATATGCCTGCCGCGTGGCATAATCCGTGTCCCGTGGTAAAGAATAATTCATTAAGAAATTTAGGGCCCAG CTCCTTTCCGTTGGGTTGGTCTTCAAAAGTTAACTAATGGCGGCAGCAGCACGATGATCAATGGAAAATTATTGAAC AGAGCTCAGGGCTGCTTCAGACCAGCTCAAGGAGCCACCTGGGTGGAGAGCAGTCAGACCCACCCACCCCCTTAGAT TTGTCAGCATTCCCTGAAACACAGTCAGACGGTGTTGATATGAGGTGTCTGCTGGGCGTGGACAGTGGGGTGGATGG GGTACTGCCCTAGTTTGGGAAGCAGCTTATTTTTAGCCTTGTAGAAGCAGCGTGAAGTGTCTACTGGTAAAGCCAGC ACGACTTCTCCTCCCTTCTGAACACCAGCAAGAAGCTTCTAGATGTTTCTGATGAACAAATAGGAGTTCCTGTACTG GCTGAGAGGGGCAGCCTACATACTTAGGTTCTTTGGGGAGGTCAGTGCGTTTTTAGCAACCATTGGCGCATTGAGCA TGAAATGGCGTGGAGCTGAAACTCACTACCAACTGCAGTGAAACCAACAAATGGAAGACGTGAACTGATGCTGGCCC TAATGACTCTGGGCAAGACGAGGGACATTATTGTTTTGCATGCATTGATAATGTGTCATCTGGAATCAGATTTTCTA CTTTCATGTTGTTCGATAACAAGGTTGTGCCCTGTGAGTCTGGGATTGTGTGAGGCGAGCGTGCGTATTCATTCTAC TGCAGAGGAGGCTGGGGAGGAATCAGAGCATCCTGTGTCTGCAAGGTCTGGCGTGTTTCTAAGGGAAGGCAGAGAAA GCCAGTGCTCCAAAGACAGCAGAGCAGACGCGAGGTTTGCAGGCATGAGGATGACAAATGAAATTCGCAGAATTCCT CAGACTTGATTTCCCTCCCTTCTATAATTAACCTCAGGGATACCTTGACTGCGCTGAGGTCCATGTGCTTGAGCACT TGCTGTCCTGCTTGCCCTGCCCTCCTCTGATTTTTTTTTGCACAGCATGATTCTATTTCTGCTATTGTTCAGAAACC ATTAGCACCTATTCAGAAGTCAGTTATTCAGCTTTGAGCTGAATGACAATTATTTGAATGGTAAAGGGTGTTAATGT ATAATTATTCACATTCATACCAGGACAGTCACAGTTTAATCATTTCTCTGTAGGCTTCCTTAAGTTTGTGCTTAAAG TCTTAGGCATAATTCTTTTGTTAAATAAGTTTAGGAGGCTCTAAGTATTGGGAAAGTGAGTATAAATACGCCTCTGG GGGAGCTTCAAGGATGTGGTGCCTCACCTCCAGGAGCGAGCCTCCTAGGTGAAACTGTCACTAGGCGCCCCACACTT CAGCCAGGAGGAAGGTCCATTTCCGAGGGCTGGTAGCATAATCTGGCCTCCAGGTGAGCTCTGAGGTCGTGTTCTGG CGTGCCCAGGACTAGATGGAGCCTACCGGGGCATTCCTATGTGTGACACAGAGCCCTGCCAGGCCCGGTCGGGCTCA GAGTGGGCTTAAATTAACCCTTCAGACCCTCGATTCCTCTGGACCCCTCTTTAAGATGATTGACGACCCCAAAGAGC TTTTGATGATAAGGGCTATATTGTGCACATTTGCCATATTGGAAATTAAACCTAAGACTTTTTTTTTTTTTTTTTTT TTTTTGCGACGGAGTCTCGCTCTGTTGCCCAAGCTTGAGTGCAGTGACACGATCTCGGCTTACTGCAACATCTGCCT CCTGGGTTCAAGAGATTCTTCTGCCTCAGCTTCATACACCCTGCATATGCCGGCTGCCTCCTCAAGCAGTACAGCTG GGATTACAGGCACCCGCCACCACACCCGGCTGATTTTAGTGTTTTTAGTAGAGTTGCGGTTTCACCATTTTGGCCAG GCTGGTCTTGAACTCCTGATCTTGTGATCCACCTGCCTCGGCCTCCCAAAGTGCTGGGATTACAGGCCTGAGCCACC ACGCCCTGCCCTGAGACTTTTTAAATGTATTAACTTATTTAAAAATAACAACAGTAAACCTATGGCAGGATATCATA GGTAATATGCTTCTGGAAATGAGTTTTCCAAGACAGACACTTGAGGAGAGTGGCACTGTTGTCCATTTTTGCAGAAT GGAATGCCTTGATGCCTGCCTCAGTGGAAGACAGCTGGGTTCTCAGATCTGCCTCTGCGTTTAATCCGTTGTGTTGT CACATCACGTAGCCCCCAGAAAACTCTTTTGTCACCTGTAAGAGAATGAGAATCTAAAAGGCAAATAACATCCTAGT ATTGCTATGGAAATGATTTTGACCTCACAAACCCCCTAAAGGGGTCTCAGTGACCCCTGATGATCCCTAGACCACAT TTTGAGAATCTCTGTTCTAAAAAGTCCATTTCTTTTACTCTAAAATATTACTCACATTGCCTGATATGTTGTGGGCT CCTCCTTTTTTAAATTTTTTTTATTATACTTTAAGTTCTAGGGTACATGTACACAATGTGCAGGTTTGTTACGTAGG TATACATGTGCTATGTTGGTGTGCTGCACCCATCTATTCGTCATTTATATTAGGTATTTCTCCTAATGCTATCCCTC CCCCAACCCCCCACCCCCCGACAGGCCCCAGTGTGTGATGTTCCCTGCCCTGTGTCCATGTGTTCTCATTGTTCAGT TCCCACCTGTGAGTGAGAACATGCGGTGTTTGGTTTTCTGTCCTTGTGATAGTTTGCTTAGAATGATGGTTTCCAAC TTCATCCATGTCCCTGCAAAGGACATGAACTCATCCTTTTTTATGGCTGCATGGTATTCCATAGTGTACACATGCCA CATTTTCTTTTTTTTTGGCATTTTTTTTAATTTATTATTATTATACTTTAAGTTTTAGGGTGCATGTGCATAATGTG CAGGTTAGTTACATATGTATACATGTGCCATGCTTGTGCACTGCACCCACTAACTCGTCATCTAGCATTAGGTATAT CTCCCAATGCTATCCCTCCCCCGTCCCCCCACCCCACAACAGTCCCCAGAATGTGATGTTCGCCTTCCTGTGTCCAT GTGTTCTCATTGTTCAGTTCCCACCTATGAGTGAGAATATGCAGTGTTTGGTTTTTTGTTCTTGCGATAGTTTACTG AGAATGATGATTTCCAATTTCATCCATGTCCCTACAAAGGACATGAACTCATCATTTTTTATGGCTGCATAGTATTC CATGGTGTATATGTGCCACATTTTCTTAATCCAGTCTATCATTGTTGGACATTTGGGTTGGTTCCAAGTCTTTGCTA TTGTAAATAATGCCGCAATAAACATACGTGTGCACGTGTCTTTATAGCAGCATGATTTATAGTCCTTTGGGTATATA CCCAGTAATGGGATGGCTGGGTCAAATGGTATTTCTAGTTCTAGATCCCTGAGGAATCGCCACACTGACTTCCACAA TGGTTGAACTAGTTTACAGTCCCACCAACAGTGTAAAAGTGTTCCTATTTCTCCACATCCTCTCCAGCACCTGTTGT TTCCTGACTTTTTAATGATTGCCATTCTAACTGGTGTGAGACGGTATCTCATTGTGGTTTGGATTTGCATTTCTCTG ATGGCCAGTGATGGTGAGCATTTTTTCATGTGTTTTTTGGCTGCATAAATGTCTACTTTTGAGAAGTGTCTGTTCAT GTCCTTCGCCCACTTTTTTGATGGGGTTGTTTGTTTTTTTCTTGTAAATTTGTTTGAGTTCATTGTAGATTCTGGAT ATTAGCCCTTTGTCAGATGAGTAGGTTGCGAAAATTTTCTCCCATTTTGTAGGTTGCCTGTTCACTCTGATGGTAGT TTCTTTTGCTGTGCAGAAGCTCTTTAGTTTAATTAGATCCCATTTGTCAATTTTGTCTTTTGTTGCCATTGCTTTTG GTGTCTTAGACATGAAGTCCTTGCCCATGCCTATGTCCTGAATGGTAATGCCTAGGTTTTCTTCTAGGTTTTTTATG GTTTTAGGTCTAACGTTTAAGTCTTTAATCCATCTTGAATTGATTTTTGTATAAGGTGTAAGGAAGGGATCCAGTTT CAACTTTCTACATATGGCTAGCCAGTTTTCCCAGCACCATTTATTAAATAGGGAATACTTTCCCCATTGCTTGTTTT TGTCAGGTTTGTCAAAGATCAGATAGTTGTAGATATGCGGCGTTATTTCTGAGGACTCTGTTCTGTTCCATTGGTCT ATATCTCTGTTTTGGTACCAGTACCATGCTGTTTTGGTTACTGTAGCCTTGTAGTATAGTTTGAAGTCAGGTAGTGT GATGCCTCCAGCTTTGTTCTTTTGGCTTAGGATTGACTTGGCGATGCGGGCTCTTTTTTGGTTCCATACGAACTTTA AAGTAGTTTTTTCCAATTCTGTGAAGAAAGTCATTGGTAGCTTGATGGGGATGGCACTGAATCTGTAAATTACCTTG GGCAGTATGGCCATTTTCACGATATTGATTCTTCCTACCCATGAGCATGGAATGTTCTTCCATTTGTTTGTATCCTC TTTTATTTCATTGAGCAGTGGTTTGTAGTTCTCCTTGAAGAGGTCCTTCACATCCCTTGTAAGTTGGATTCCTAGGT ATTTTATTCTCTTTGAAGTGATTGTGAATGGGAGTTCACTCATGATTTGGCTCTCTGTTTGTCTGTTATTGGTGTAT AAGAATGCTTGTGATTTTTGTACATTGATTTTGTATCCTGAGACTTTGCTGAAGTTGCTTTTCAGCTTAAGGAGATT TTGGGCTGAGACAGTGGGGTCTTCTGGATATACAATCATGTCATCTGCAAACAGGGACAATTTTACTTCCTCTTTTC CTAATTGAATACCCTTTATTTCCTTCTCCTGCCTGATTGCCCTGGCCAGAACTTCCAACACTATGTTGAATAGGAGT GGTGAGAGAGGGTATCCCTGTCTTGTGCCAGTTTTCAAAGGGAATGCTTCCAGTTTTTGCCCATTCAGTATGATATT GGCTGTGGGTTTGTCATAGATAGCACTTATTATTTTGAGATACGTCCCATCAATACCTAATTTATTGAGAGTTTTTA GCATGAAGGGTTGTTGAATTTTGTCAAAGGCCTTTTCTGCATCTATTGAGATAATCATGTAGTTTTTGTCTTTGGTT CTGTTTATATGCTGGATTACATTTATTGATTTGCATATATTGAACCAGCCTTGCATCCCAGGGATGAAGCCCACTTG ATCATGGTGGATCAGCTTTTTGATGTGCTGCTGGATTCGGTTTGCCAGTATTTTATTGAGGATTTTTGCATCAATGT TAATTAAGGATATTGGTCTAAAATTCTCTTTTTTGGTTGTGTCTCTGCCCAGCTTTGGTATCAGGATGATGCTGGCC TCATAAAATGAGTTAGGGAGGATTCCCTCCTTTTCTATTGATTGGAATAGTTTCAGAAGCAATGGTACCAGTTCCTC CTTGTACCTCTGGTAGAATTCGGCTGTGAATCCATCTGGTCCTGGACTCTTTTTGTTGATAAGCTATTGATTATTGC CACAATTTCAGCTCCTGTTATTGGTCTATTCAGAGATTCAACTTCTTCCTGGTTTAGTCTTGGGAGAGTGTATGTGT CAAGGAATTTATCCATTTCTTCTAGATTTTCTAGTTTATTTGTGTAGAGGTGTTTGTAGTATTCTCTGATGGTAGTT TGTATTTCTGTGGGATTGGTGATGATATCCCCTTTATCATTTTTTATTGCGTCTATTTGATTCTTCTCTCTCTTTTT CTTTATTAGTCTTGCTAGCGGTCTATCAATTTTGTTGATCCTTTCAAAAAGCCAACTCCTGGATTCGTTAATTTTTT GAAGGGTTTTTTGTGTCTCTATTTCCTTCAGTTCTGCTCTGATTTTAGTTATTTCTTGCCTTCTGCTAGCTTTTGAA TGTGTTTGCTCTTGCTTTTCTAGTTCTTTTAATTGTGATGTTAGGGTGTCAATTTTGGATCTTTCCTGCTTTCTCTT GTGGGCATTTAGTGCTATAAATTTCCCTCTACACACTGCTTTGAATGCGTCCCAGAGATTCTGGTATGTTGTGTCTT TGTTCTCGTTGGTTTCAAAGAACATCTTTATTTCTGCCTTCATTTCATTATGTACCCAGTAGTCATTCAGGAGCAGG TTGTTCAGTTTCCATGTAGTTGAGCGGTTTTGAGTGAGATTCTTAATCCTGAGTTCTAGTTTGATTGCACTGTGGTC TGAGAGATAGTTTGTTATAATTTCTGTTCTTTTACATTTGCTGAGGAGAGCTTTACTTCCAAGTATGTGGTCAATTT TGGAATAGGTGTGGTGTGGTGCTGAAAAAAATGTATATTCTGTTGATTTGGGGTGGAGAGTTCTGTAGATGTCTATT AGGTCCTCTTGGTGCAGAGCTGAGTTCAATTCCTGGGTATCCTTGTTGACTTTCTGTCTCGTTGATCTGTCTAATGT TGACAGTGGGGTGTTAAAGTCTCCCATTATTAATGTGTGGGAGTCTAAGTCTCTTTGTAGGTCACTCAGGACTTGCT TTACGAATCTGGGTGCTCCTGTATTGGGTGCATATATATTTAGGATAGTTAGCTCTTCTTGTTGAATTGATCCCTTT ACCATTAAGTAATGGCCTTCTTTGTCTCTTTTGATCTTTGTTGGTTTAAAGTGTGTTTTATCAGAGACTAGGATTGC AACCCCTGCCTTTTTTTGTTTTCCATTTGCTTGGTAGATCTTCCTCCATCCTTTTATTTTGAGCCTGTGTGTGTCTC TGCCCGTGAGATGGGTTTCCTGAATACAGCACACTGATGGGTCTTGACTCTTTATCCAATTTGCCAGTCTGTGTCTT TTAATTGGAGCATTTAGTCCATTTACATTTAAAGTTAATATTGTTATGTGTGAATTTGATCCTGTCATTATGATGTT AGCTGGTTATTTTGCTCGTTAGTTGATGCAGTTTTTTCCTAGTCTTGATGGTCTACATTTTGGCATGATTTTGCAGC GGCTGGTACCGGTTGTTCCTTTCCATGTTTAGTGCTTCCTTCAGGAGCTCTTTTAGGGCAGGCCTGGTGGTGACAAA ATCTCTCAGCATTTGCTTGTCTGTAAAGTATTTTATTTCTCCTTCACTTAAGAAGCTTAGTTTGGCTGGATATGAAA TTCTGGGTTGAAAATTCTTTTCTTTAAGAATGTTGAATATTGGCCCCCATTCTCTTCTGGCTTGTAGAGTTTCTGCC AAGAGATCTGCTGTTAGTCTGATGGGCTTCCCTTTGAGGGTAACCCGACCTTTCTCTCTGGCTGCCCTTAACATTTT TCCTTCATTTCAACTTTGGTGAATCTGACAATTATGTGTCTTGGAGTTGCTCTTCTCGAGGAGTATCTTTGTGGCAT TCTCTGTATTTCCTGAATCTGAATGTTGGCCTGCCTTGCTAGATTGGGGAAGTTCTCCTGGATAATATCCTGCAGAG TGTTTTCCAACTTGGTTCCATTCTCCCTGTCACTTTCAGGTACACCAATCAGACATAGATTTGGTATTTTCACATAG TCCCATATTTCTTGGAGGCTGTGCGCGTTTCTTTTTATTCTTTTTTCTCTAAACTTCTCGCTTCATTTCATTCATTT CATCTTCCATCGCTGATACCCTTTCTTCCAGTTGATTGCATCGGCTCCTGAGGCTTCTGCATTCTTCACGTAGTTCT CGAGCCTTGGTTTTCAGCTCCATCAGCTCCTTTAAGCACTTCTCTGTATTGGTTACTCTAGTTATACATTCTTCTAA ATTTTTTTCAAAGTTTTCAACTTCTTTGCCTTTGGTTTGAATGTCCTCCCGTAGCTCGGAGTAATTTGATCGTCTGA AGCCTTCTTCTCTCAACTCGTCAAAGTCATTCTCCATCCAGCTTTGTTCCGTTGCTGGTGAGGAACTGCGTTCCTTT GGAGGAGGAGAGGCGCTCTGCTTTTTAGAGTTTCCAGTTTTTCTGCTCTGTTTTTTCCCCATCTTTGTGGTTTTATC TACCTTTGGTCTTTGATGATGGTGATGTACAGATGGGTTTTTGGTGTGGATGTCCTTTCTGTTTGTTAGTTTTCCTT CTAACATACAGGACCCTCAGCTGCAGGTCTGTTGGAGTACCCGGCCGTGTGAGGTGTCAGTCTGGCCCTGCTGGGGG GTGCCTCCCAGTTAGGCTGCTTGAGGGTCAGGGGTCAGGGACCCACTTGAGGAGGCAGTCTGCCCGTTCTCAGATCT GTAGCTGCGTGCTGGGAGAACCACTGCTCTCTTCAAAGCTGTCAGACAGGGACATTTAAGTCTGCAGAGGTTACTGC TGTCTTTTTGTTGGTCTGTGCCCTGCCCCCAGAGGTGGAGCCTACAGAGGCAGGCAGGCCTCCTTGAGCTGTGGTGG GCTCCACCCAGTTCGAGCTTCCCGGCTGCGTTGTTAACCTAAGCAAGCCTGGGCAATGGCGGGTGCCCCTCCCCAAG CCTCGCTGCCACCTTGCAGTTTGATCTCAGACTGCTGTGCTAGCAATCAGCGAGACTCCGTGGGCATAGGACCCTCC AAGCCAGGTGCGGGATATAATCTCCTGGTGCGCCGTTTTTTAAGCCCGTCGGAAAAGGGCAGTATTCAGGTGGGAGT GACCCGATTTTCCAGGTGCCGTCTGTCACCAATTTCTTTGACTAGGAAAGGGAACTCCCTGACCCCTTGCGCTTCCG GAGTGAGGCAATGCCTCGCCCTGCTTTGGCTCGCGCACGGTGCACGCACCCACTGACCTGCGCCCACTGTCTGGCAC TCCCTAGTGAGATGAATCCGGTACCTCAGATGGAAATGCAGAAATCACCCATCTTCTGCGTCGCTCACACTGGGAGC TGTAGACCAGAGCTGTTCCTATTCGGCCATCTTGGCTCCTCCCCATGTGCCACATTTTCTTAATCCACATCATTGAT GGGCATTTGGGTTGGTTCCAAGTCTTTGCTATTGTGAATAGTGCCGCAGTAAACATACATGTGCATGTGTCTTTATA GTAGCATGATTCACAGCTGAATTCTACCAGAGGTACAAAGAAGAGCTGGTACCATTCCTTCTGAAACTATTCCAATG AATAGAAAAAGAGGGAATCCTCACTAACTCATTTTATGAGGCCAGCATCATCCTAATACCAAAGGCTGGCAGAGACA CAACAAAAAAAGAGAATTTTAGGCCAATATCCCTGATGAACATTGATGTGAAAATCTTCAATAAAATGCTGGCAAAC CGAATCCAGCAGCACATCAAAAAGTTTATCCACCACCATCAAGTCGGCTTCATCCCTGGGATCCAAGGCTGGTTCAA CATACACAAATCAATAAACATGTTCCATCACGTAAACAGAACCAAAGACAAAAACCATATGATTATCTCAATAGATG CAGAAAAGGCCTTCAACAAAATTCAGCAGCGCTTCATGCTAAAAACTCTCAGTAAACTAGGTATTGACGGAACGTAT CTCAAATAATAAGAGCTATTTATGACAAACCCACAGCCAATATCATACTGAATGGGCAAAACTGGAAACATTCACTT TGAAAATCGGCACAAGACAAGGATGCAGTCTCTCACCACTCCTGTTCAACATACTGCTGGAAGTTCTGGCTAGGGCA ATCAGGCAAGAGAAAGAAATAAAGGGTATTCAATTAGGAAAAGAGGAAGTCAGTTGTCTCTGTTTGCAGATGACATG ATTCTATATTTAGAAAACCCCATTGTCTCAGCCCAAAATCTCCTCTTTTTTATTTTCGAAAAAATTACAGCAGAGTT CACTCTTCTTGGTGTGCAGCAGGAGGTGCACGTGCTGTGCTTGTCCATCAGTGTCTGCTTTGTGCATCTCTGTTGAC CTGCCTCTAACACTGGGACGAGCATCAGCCAAATCAGAACCAGGTCAGAGCCCCTACGCCCTGGTCAGATCAGAACC AGGTCAGAACTCCCCTACACCACAGTCAGATCAGAACCAGGTCAGAACCCCTATACCAAGCTTGTCCAATCCTCAGC CTGCGAGCCACCTGCAGCCCAGGATAGCTTTGAATGTGGCCCAACATAAATTCATAAACTTTCTTAACACATTATGA GTTTTTTTATGATTTTTTTTAACTCCTCAGCTATCATTAGTGTTAGTTATTTTATATGTGGCCTAAGACAATTCTTC CAGTATGGCTCAGGGAAGCCAAAAGATTAGACACCCCTGCCCTACACCTTGGTCAGATCAGATCCAGGTCAGAGGCC CTACATCCTGTCCCCACAGTGGACAGGTTGGAAATGTAAGGTCGTGTGGCCACTGGGGGCCCAGGATGGGCTCTGCC TCAGAGGCACTTCCTTCCACCTGGCCTGTTTTAATCAACAGTCATTAAAGGGAAATCTAAAGATGTTTAAAACGTTG TGTATTTTCTCGTTAGCAGAGCCAAGAATCAGTCTGCAGGGAGCAAAGAGCTTGAGTGTGGAGTTAGAAGTCGGGGT CCGGGAAAGGGTGCTTGTTTCTGAGGCTAACATGCATCAGGTGCTCCAAGTGGGCTCTCATTGACCCCAGGATGCCA TCAGCCCCCTTGCCCTGCCTGGAGTATATCACATGGGCCCTGGGTGCCTGGGCCTGTGGAGCCACAGCTGCTGAGGT CCGGTGTTTTTAAAGCCTGAGAGAGGCCATCTGGGGGTAGCACTACAGGAGATGGGAAGAGGCCCCCAGGAAACCAG GGAGAGCAAGAAGGTCTTCTAAGGACTCATGTCCTCTTACCAACTACCATGTAGTTAAGAATGCCTTACGAAGGGGG ACGGGAGAGTCAAGGGATAGAAAATCAGTCCTTAAAATCTGCACTGTCCTTTCCACATACAAAGCACCTTTCACACA GGTGAGCTCATTTACCCTCAGATGGCACTTACTGTTGTGGGAACCTCATTTTACCAGGAGGAAACGGAGGGGCGACA GGACAGCGTTCTAGGAAGTAGCTCAGCTGTGGACATTTTGTGGTGAGCTCTCAGGGACAGCAGCAGGCATGGGATGC CTGCAGAGAGGCTTTCAGGAGAGCGCTGGCCCCACCTGCAGGCCCCCAGGGCGACCTTGGATAGGCTGCCGTCTGGG CCTGGGAGACTTCTCACCTGCAGAGTGGATGATGGGGGACCTTGAGGACTGAACAGGAGAAAGCACACTGAACCGAG CCTGGTACCTAGTAAGCACTCAGTAAGTCTTGGCACTTACTGTGATAATTGCATTTTAAGCCCAGCTACCAAAATTT CCAGACCTACGTTACCATCTTCAAATGAGTGCCCCTACCCGTTTCCCTAACGTTGCTCAAACTTGCATTTTCTGAGG AGCGTCTGTTGGATGCCTTTGGCCCTTGCACCACGTACAGCTTTCCACGCTTATTGGCTGGCCACTGGGTACCAAGG ACCATGGCGGGCACTCTGGAATGCTCTGTTTTGGCAAACTTTCTTCCCCAAGAACAGATTTGACTTTTGAAAAGTGG TCTGAAAGTCAGCCTCCCCAGCCCTGGTCACAGCAGAAATAGTTTTGGACCCAGGTGAGTTGAGTCCACAAAATAAC AGGCCGTGTCTACTTGAGCTGAAGCCTTATCAGTTGGCCCAGTAGGTCCCATCTGAAGAAGCCCTGTATTTTTGAAA AATGTTAACTCTCCCTAAAAGGTTACATTGAACGGCAACACCCGGGAGGCTGCTGTTGGCCTTGCCAGCAAGCTGAA CCTGCAGTTGAGTGGCCACGCGTGTGGTGCGAGGCGTGCAGCCCATCCCTGAGGGCTCCCCTGCATGGGAAAGGAGG CTGGGAGGTTCGCCAGGACTGCATGTGGCCCTCGCACCAAGAACCTGACCCATGGTGGAGGTGGTGAGTGCTGTGTG GTCGGCCTTGATGTTTATACGAGAGTCTCTGAGGTGTAGACATCAGTGTCTCCATTTTGGGGAGAGGGAGGCGGTTT TCTTCCCAGCTTACCATTACCACTGTGTTCGAGGAGGGACTGATATTTCGAGTGGGGTCACCATCACACTCACACCA CCAAGGCTGCTTTCCACCACTCAGCTTTCTCATGCATCCCGTTTCCCAGAGGTGGCCAGTGCCTGGGCCCTGAGTCG GTTGAGACTCATCCAGAGCAGAGCTGCAGAAGCTGGGCTGGCTCCAGACAGAGATGACCCAAAGCCTGTGTCAGCAT TTGGGTGCCTGGGCCCACCGTCCAGGCCTGAGAGAGCATCAGTGAGGCTCGGAGCCCCCATCAGCCAGGTCAGGCCG TGGCTCTGAGCCTAGTGCTCCTTTCTAAATGGGAGTTGCAACGCCAGCTTTGCAAAGTGGTTGTCAGGGCTCACTGA GGGGCTCTGGGGAGACTTTGGGGGAAGGATAATGATTGGGGAAGTAGAAATTGGCTCCCAAAATGCTAGAGCAGGAC TTAAGGGACTGATGGCCAGGCACCTGCCCAGTGTGGCATCCTCCCATCGTTGGCTCGAGCGCTGAGCTGCACCTCTG TGTCAGGACTTGCTCTCAGTGTTCTGTCTGGGGAGGTCATGTGCCTACTGGGTGGGGCAGGTCCTTGTGTGGGAGGA GCCAGAAACTCCCTTTTTTCTTAAAAGTTATTGGGCCAGAGGTCATGAATTCACCCTTAGCCTATGATCTCCAGTAA CTCTACCATCCGCAGGAAAATGACTTCACACCATGCGTAGGGCTGGAAGGAGCACTGGGATGGAGTCCCACCTCGGT GCGCGACCCGGGGCGTTCAGCAGTGCCCCAGGACCGCAGCAGCCCATGCTCAGAGCCAGGTCCTCAGGGCCGGGGCA TTCAGCAGTGCCCCACTACCATAGCAGCCCATGCTCAGAGCCAGGTCATCAGGGCCAGGGCATTCAGCAGTGCCCCA CGGCTGCAGCGGCCCATGCTCAGAGCCAGGTCCTCAGGGCTGGGGCGTTCAGCAGTGCCCCACTACCACAGCAGCCC ATGCTCAGAGCCAGGTCATCAGGGCCGGGGCATTCAGCAGTGCCCCACGGCTGCAGCGGCCCATGCTCAGAGCCAGG TCCTCAGGGCTGGGGCGTTCAGCAGTGCCCCACTACCATAGCAGCCCATGCTCAGAGCCAGGTCATCAGGGCCGGGG CATTCAGCAGTGCCTCACAACTGCAACGGCCCATGCTCAGAGCCAGGTTCTCAGGGCCGGGGCGTTCAGCAGTGCCG CACGACTGCAGTGGCCCATGCTCAGAGCCAGGTCTTCAGGGCCGGGGCATTCAGCACTGCCCCACCACCGCAGCTGC CCATGCTCAGAGCCAGGTCATCAGGGCCGGAAGATGGCAACTTGATGCTTAGCCTTCAAAGAGGTGGTAAACGATAA AAAAACGGAGCCATGCCTGGATTCAAGAGCCTCTGAAATCCTACCACCTAATCACTGTCTTCCCTAATTTCCCCTTG GCCGCTATGTGGGTGGTATATGGTGGGCTGTTGGGTGGAGCGGGGGGGGTCGTTACTGATCTATCTGGAATACCCGC TTTATCTCTACCCAGCAAACTGCTACCCATCCAAGACCCAGGCCCACCTGTCACTTCCACTCTGGCACCTCGTTCGC CATCACCTCACTCAGGAGGGTTCCACTGGTGCTTGCCGGCGAACGCCACCATTTTTTGCAGCATATCTCATAGCTTG TTGGAAACCCACGTTGAGTTGGGGCTTTGTTCATCCACAGGTGCTGTCACGCATCCGTTTTCAGGAGACAGAGATGT CCTCTGTGGCCCTCCGTTGGCCGACCCTGTTGGGTGCAGCAGCTGTCAGTGGACAGAGGTGCATCAGTGCGTTGGTT GGTGGCATCGTGGCCTCACCGGCCCACAGTTCTGACAGAGGTGTCTGTGTTTCACCAACTCAGTCTGTCCCTGCCCA TTGTCCGCACACTCATTCCCTCCATGCCCCAGTCTGGCACCCTCAAGTGCAAGTGCGTGCATTTAATTCACGTAGTG TGTTGGTTGGGTGCACTCCTGGACCTTTGGGGAGACTGTCTTCTTCCATGTTAGCTGCTTCCTTCTTCCCTGTGGGG TCATAGAGGTCACAGCTGACATGCTGAAACAAATGCCATTGTTTTCACTTAAACCACATTGTATTCCATAGCCGCTG TGTCATATGCGTATATATAATAGTGTGTAAGGCGTATGGAAAGAAGAGATTGTGTCCAGACTGGCTACAGATGGTCA TCCTAATGGCGGAAGCAGCCAGGCCAGTTGGGCTTGACTCTGGAGCTTTGGAGGGCCATCCTCAGTGTATTTGTTTT TATTTGTGGAGGCCCCGCAGCGCTCATGCTGGCCATGCCTGGCTGGGATGGGATGACCTGGTGAGGACAGAGGAGGA CAGCCTCTGCTTCCATGATGAATAAAACAGCATCTGGCTGGGTAGGAGGAAAGCCTGTGCTTGTTCAGCCTCAAGTA CGTGTTTGAAAGCTTGCCCCAAAGACATTCCAAAAACCAGGCAGTGGTGGTGTCCTGTCCTTCTAGAGGCCAGGTTG TCCAGGCCCAGGGGAGTCTTGCTTAGGGGTCAGTGAGCGCTGGGACTCAGTCTGGCTCCTGGCAAGCCCAGAGAACC CCACTGCTGAGTCTCTACACAAGGCATTGGACGGGGCTGCTGTGGGTGTGCGCCAGGCCACCTCCATGCCTGGGCAG CTGTGCACAAGCTGGCTATGAGCTCCATGGCATACAGAGCACGTCCAAACTAACCTGGTAGCCCAGTCCCATGGAAA GTGTGTGACATCGTGTCCCTGCCTTCGGAGTGAGCAAGCCACAAGCCAGATGTCAGGGGCTGTTCCTTCTGCAGGTC ACCCACTGGCTGGCCAAGCACCGTGAAGCTGGGCGCACCTCCTCCAGGGACCATACTAAAGCAGCTAGCAGCACCTT CCCCCTGCTGTGAACCCCATGCCTGACCTGAGCCTGAGCCCTCACAGGCCTGGCAAAGGCTGCCTGCAGAGGCCTGG GCCACACAGGCCCTATGGCTTTCCTGCGTGCCACGGAGAGCCAGCCAGTGGGGCTGCAGGCACCTCCTCCCGCTCAC TCTCAGTGTGCGCCTCCACAGGGACCACAGACCACAGGAGCTCCTGGCCCGCCACATCGCCAGCCGTGTGCGCAAGG TTTATCAGGTTCCTCTTGGCCAGGTTGAAGTAGATGCCGGGTGGGGATCTGTAGGCCCTGGTTGTGTTTACAGAAGA TGCTGCCCTTCACTGTCTTCCAGCCTCCCAGGAAAGGATGGTCCCCAGGCTCCATGCTGACACCACCCACCTTGCTT CACCCAGTAGGCGCTTACTGTATGTCTACTCTAGGTCATCCACAGTGCTGAGTGTTGGGATAGAGCCCCGGGAGGTA GGTGCCTGGTCCTCCGTGAGTCTGGTGGAGGAGCCCTCCGTGGTGACAGCCTTCTGTGAGAGCATGGCAGGTGGTGG GGCCTCGTAGAGGGAAAGATTTTGCAGAAAATGTGACCTAGATTCCCTTTTAAATAGGAAATACTGGTCCCTAGTGT CTTGAACTTTGGGGAGAAAACTCCCTTTGATTAAAGAGAAGGTAACTTGGAAATGTGTTTTCCCTCATCTTTTTGTT AATTTATTTTTTAAATTAACATGTAGTGAAATTGGCTTTTTGTGGGGTATACAGTCCCGTGAATGTTAACGCGTGTG TGTGTTTCTGTAACCACCCCCAAAATCAGGGTGCAGAGGAGTTCCACCACCTGGACACCCCCACGCCGCCCCCTGGC AGCCACGGCCTCTCCCCACCCCCATTCCAGCTCTCAGCAACTGCGACGTGTTCTCCAACACCGTGGCTTTGTGTTTT TGAGAATGTCTTATAAATGGAATCAGACAGTATTTAACCTTTGGAGCCTGGCTTTTTTCACTCAGGTTAATGCCTTT GAGATTCATCCAAGTTGTTGCATGTATCTACAGCTTATTCATTTTTCTTGCTGAGTGTACGCTACCGTGTGGGTATA TCTCCATTGATCTGTTTTTTATTTTGGCCATTCTAATAGATATGTAATGATATCTCATTGTTTTCATTTGCGTTTCC CCGATGGCCAGTTATGTTGAACATCTTCTTTTGTGGTTATTGACCCCTCCTTATATCCTCTTTGGGGAAGTGTCAAT TCAAGTCTTTTGCCTTTTTTTTAAGTTAGGTGGTTTGTTGAGTTTCAGTTTGGAACTGTTGAGTTTGGAAAAATCTT TATATATTCTGGATAGGATAGGACTCTTGTTGGATTTCTTATTTCCAAATATTTTCTCCTAGCTAGTAGTAGCTAGT CTTTTATTCTCTTAACACTGACTTTCAGCCGGGTGCGGAAGCTCATGCCTGTAATCCCAGCACTTTGGGAGGCCAAG GGGGCGGATCATGAGGTCAGGAGATCGACTGCATCCTGGCTGACACAGTGAAACCCTGTCTCTACTAAAAAAATACA AAAAAAATTAGCCAGGCGTGGTGGCAGGCGCCTGTAGTCCCAGCTACTCAGGAGGCTGAAGCAGGAGAATGGCGTGA ACCCGGGAGGCAGAGCTTGCAGTGAGCCGAGATCGCACCACTTTGCTCCAGCCTAGGGGACAGAGCGAGACTCTGTC TCAAAAAAAAAAAAAAAAACAAACAAAAAAAAACACCTGACTTTCATAGACCAAACTTTTTCACAAACAAGTTTTTT TTCCCTTTTTAATGAAGTTCAATTTATCTCTCTCTTTTTTTTATCATGATTTGAAGGACATATCTAACAACTGTTTG CCTAACCCCAGGTCATGAAGATTTGTCATATAGTTTCTTTTTTTTTTTAGTTTAATGTTTTACATTTAAATCTATGA TATATTTTGAGTTAATTTGTTTAAGTAAGTCATGAGATTTACATCACGTTTCACTATTTGCCTATGGATATCCAATT TTCCAGCACCTCTTTTTGAAAATAATATGCTTTCTTCATTGAAATCCCTTTGCACCTTTGTCAATAAACATTTGGTC GTGCTTCAGTAGGTCTATTTTGGAAATCTCTCTTCTATTCTGTTAACTCCATCCAGTAACTCCATCAATACTCCAAC AAGCTGGGTATTTATCCCTTTGCAAATACATAATTATTATTATGTATAGTAAGTCTTGAAACCTGTCAGTGTGATTT CTCTAACCTTTTTTGTCTCTTGTAAAATATGTTGGCTAGTCTCAATCCTTTGCCCTTTCATCTAAATTTTAGAGTCA ACATGTCTATCTGCAATAAATTCTTCTAGGGTTTTTTAATTGGAATGATAATAAACCTATAGGTCAGTTTGTTAGAA TTCAAATTTTATGATATTGAGATTTCCATGAACATGATATGTCTCCTCAGTTATTCAGACATTCTTGTGATTACTTT TTCAGCATTTTGTAATTTCCAACAAACAGAGCCTATATACAAATTGTTAGATTTACATCTAAGTATTTTATTTGGTT GGAGCCATTATCAGTGGTATATAGAAAGGTGATTGATTTTTATTTAGTAACCTTGTATCTAGTGACTTTGCTAAACT CACTTATTAGTTCTAAGACTTTTCTATTATTTCCTGGGTATTTTCCCATAGACAATTATGTTGTCTGCAAATAGGGA GAGTTTGTTTCTTCTTTTCTAAAACAATTGTCTTCTATTTCCTCATCTTGCTTTTTTTGCACAGTTAGGTATGATGT TAACTGTGGGTTTTTTGCAGATACAGTTGTCAGATTGAGGATGTTACCTTCTGTTCTTAGGTTGCTGAGAGCTTTTA TCATGAATAGGTGTTAAATTGTATTAAATATTTTTTCTGACTGGATATAGATTATGTGTTTTTCTAGGATATTAATA TGATAGCTATTGTTCGTTTTTAATAGCCTTTGTTTTTAGAATAGTTTCGGATTTACAGAAAAATTGAAAAATCAGTA GGGTTCTCTGATACTGCACACCTATTTTCCCAAATTATACCATTGATTCTTATTATGATGACATTTACACTTGACAT CTTACATTAGTACGGTACATGTGTTATGATTCATGAACCATTGTTGGTACCTTAATATTAACTAAAGTCCTATGATC TTGAAGTTTTCTTAGTTTTTACCTACTGCCCTTCTTCCATTCCAGGATCCTGCCTTGGATACTGCATTCCTAGCTCC TTTGGCCCCTCTTGGCTGTGACAGGTCCTTAGTTTTAGATGGCCTAGACAGTGTTAAGGAGTACTGATTAACTATTT TGTAGAATGTTCCTCTTTTGGAATGTTTTATGCTTTTCTCATGATTTGACTGGAGTTACAGTGTGTGAGGAGAAAAA CCACATAGGTAAGCTGTCATTTTCCTGAGATCGTATCAAGGAGTCATGCTGTCAGTGTCATTTATCATTGTTGATGT CAACTTTGATTACCTGGCTGGGTAGTGGTGTTTGTCAGGTGACTCTATGATAAAGTTACCTTCCCCCATCTCCCCTG CGTACTGCACTCGTCCCGAAGAAGTTGCTGTACCTAACACACTTAGGAATGGGGAGTTACGCTCCTTGAAGACAGAG TATCTACATAAGTTACTGGGAATGCTTCTGCATGGGAGAGTTCTTCTCCCTCACCTATTTCTGTATTCAGTCATTTT TTTATGTTAGTATGGACTCAGGGATATTGATATTTTGGGTTGTCTTCCAATACTACTTTATTTTGTTGCTGAGATTA TCCCAGTTTTGGCCATTTGGGAGATTGTGCAGTTTGCTCCTGTGTCCCTTTGTCGTACTCTCCTCATCGTGGTTTGA CTGGAATGTTTGTCTTTGTTCTTTTGCTTCATTTCATTTTGGAGTGTGTCCTGACTTTCTGATACTGCAAGATTCTC AAGGCTCATCTTGTGTATTTCCTGTCCCAGTCCTAGGGAGGAAAAGGCCAGTTCTTGAAGGAGTCTTGTGTCTTTTA TTATAAAATGCATGAGAAACCATGGACTGGGCACTAGGTTTGCTCCTTGCTATGGGGGTGGTGTTGTTTCTAAGTCT TCTCAGTTGACAGACCACAGAAGTAGCTTTCTGTATACTAACCCACGGACAGATAAATATTGTAAACATTTCTGTAT GCAATTATCTGTGTCTATATTAAGCTAAACATTAGTTCCTATTGATATCTGCAACTCTAATCCATTACCACATGGAT TCTTCTAGCCTCCTCTCCTTACTTATCTGTAAAATCCCACTCACAATGAGAAACCTGGCTCACACTTTCTGCCATCC ATTTACTTAATTATTCAAGCCCAGTATACATATAGAATAGTATCAGAGAGTTTAACACCTGTGCTCATGGGAAACAA CTTTATCAACTAGAGTTCAGTATTTATGTACAGTTCCTTTTGCCTTTAGTCTTACAGACTCCACTCACTTTCAAGTT ACTTTTGGAGAAATCAGCTCCCCTCCCCCATTCAGTGAGGACGTTTTATAAGTTTATAATACAGTCAGATTCTCTTG TCACAGTCTGTGTTTCTTCCCAGGATCCCCTGATCTCCTAAATATTTTGTTGTTGTTAATTTACATACATTGCTTAG ATTCACTCTTTGTGCTGTAAGCTTCTATGGCCTTTGATAGATGTATGATATCATGTTTTCACCATTACAGTAACATG CAGAATAATTTTACACCCTAAAAATCCCCCTGTGCTCCACCTATTCAGTCCCCCGCTCCTTCTGAAAGTGCCCCGGC AACCACTTATCTTTTTTCTGTCTATATAGTTTTGCTTCTTCCAGAATGTCATCTAGTTGGGATCATACAGTATGTAG CCTTTTCGGCTGGCTTCCTTTGCTTAGCAATATACATTTAGTTTTCTCCGTGTCTTCTGCATAGCTCATTTTTTTCA CTGAATAATATTCCATTGTATGGATGTACCATAGTTGTTTATCCGTTCACCTATCGAACAACATTTTGGTCACTTAC AACTTTTGGCAGTGTTAATAAAGCTGCTATAAACATTCACAGGCAGGTTTTTGTGAGGATGTCAGTTTTCAAATCAG TAGAGTAAATACCTAGGTGTGCAATTGCCAGATCACATGGTAAGGCTCTGTCTTTGAAAGAAATTGCAAAACAGTCT TCCAAAGTGGCTTTACCATTTTGCATTCCCATCAGCAATGATTAAGCATCCTTTTTGCTCCACATCCTTGCCAGCAG TTTTTGGTACTGTCACTGTTTGGATTTTTAGCCATTCTAAAAGCCATTGTAATCATATTCATTATTTTAGTGTGCAG CTCCTAAAGACAAATTATGTTAAATATCTTTCCTTGTGCTTATTTGTCACCTATATATATTCTTTGGTGAAGTGTCT GTTAAGACTTGCCCATTTTTTAATTGGGTTGTTTGACTTTTTTCTTGTTGAATTTTAAGAGTCTTTTTATAATATAT TTTAGATGCAAGTCTTTTTTCAGATATGTGTCTTACATATATCTTCTCCTAGTCTGCCTCTCGTTTTCAGAGTCTCA GCATCTTTTGTAGTAAAGAAGTTTTTAATTTTAATAAACTCCAGCTTATTCATTTTTTTCTTTCATGAATCATGTTG TATCTAAAAACTCTTCTCCCAACCTAAGGTTGCTTAGATTTTCTCCTGTGTTATATTCTGAAAGTTTATGATTTTCC CTTTTAGATTTAGGTCCATGATCCATTTTGAGTTCATTTTTGAGAAAGGTATAAAGTCTGTATCTGGATTCACTTCT TTTTCGTGTGATGTCTAATTGTTCCAGGGCCATTTGTTTAAAAGACTATGCTTTCTCCATTGAATTGCTTTTTGCTT TTTTTTGTCAAAGATCTTTGTCAAAGTCTAAATTTGGCCCTTGTATTTATGGGTTCCACATCCATGTATTCAACCAA CTGCAGATAAAAAATGTTCAACAACAAGGAAAGAATGGTTGCATCTTTACTGAAAGCGTACAGACTTTTTCTCCTTG TCATTATCCTCTCAACAATACAGTATAACAGCTATTTACATAGCATTTACATTATTCTAGATATCCTAAGTAATCTA GAAATGATTTAAAGTCTACAGGAGGATGTGCAAGGGTTATATGCAAATACTATGCCATTTTATATCAGGAACTTAAG CATCCATGGATTTTGGTATTCTAGAAGGGTCCTGGAACCAATACCCAATGGATACCAAGGAATGACTGTATATCTGT GTGTGCCTGTTTCATTGACCTTTGTGTCTGTTCTTTTACCAATGCCACCCTGTCTTGATTACTGTAGTTTCATAATG TAGATTTATTTTCAAATATTGAATCAGCCTTGCTTTCCTGGGACAAACCCCACTTAATTTTGATGTCTTGTTCTTTT TATATATTGCTGGATTTGATTTGCCAATATTTTGTTGAGGATTTTTACCCTGTGTTCATAAGAGATACTGAACTGTA CTCTTTTGGTTTCTGACTTTTTCTGGTTTTGATGGTGGAGTAATGCTGGTCTCATAAAACACTTTAGAAAATGTTCC TTTCTCTGGAAAAAATTATGTCAAATCAATCTTATTTTTAAAACATTTTATAGAATTCACTGGTAAAACCATCTTGG CCCTAGAGGTTTCTTTTTCAGAAGATTGTAAGCTATGAATTCAACTTCTTTAATAATTATAGGAGTATTCAGTTTCA TCTTGGATAAGTTTGGTAGTTTGTAGTTTTTGATGCATTGGTCTATTCGCAGTTGTTGGATTTTAGTACACAGTTAT TTGTAGGAATACCTTATTATCTCTTTAATATCTGTGGGATCTGTGGCAATATCTTCTCTTTTATTCCTGATATTGAT TATTTGTTTCTTCTTTTCTGTCAGTCTTTTAGAGATTTATAAATTTTATTGATCATTTCATGGAACTCATTTTTAAT TTCTTGGATTTTACTCTTATCATTTGTCTGTTTTCAATTTTGTTGATTTTCGCCTTTGCCTTTATTATTTCCTTTCT GGTTGTTTTGAGTTTATTTTTCTCTTCTTTTTCTGGTTTCTTAAGTTGGAAGTTTAGATTATTGAGACCTTTCTTTT TTAATATAGACACTTAATACTATAAATTTTCCTCTAAATATTGTTTTAGCTTTGTCCCATAAATTTTAATATGCTTT ATTTTCAGTTTCATTCTGTTCAGTATACTCTTTTAATTTTCCTTCAGACTTTCTCTTTGACCTATTGATTATTTAGA AGTGATATGTTTACTGTTCAAATGTTTGGAGAATTTTCTATTATGGTTTTTGTCGATTTCTAGTCTAATTCCATTAT GATCAGAGGATGTAACTTATATGATTGCAGTCATTTTGCATGTGTTAAGGTTTATGGCCCAGAAGAATATGGCATAT CTTTGTCAATGTTTTATGCACACTTGAAAAGAGTATAGATTCTACTGTTATTGGGTGGAGTGTTCTGTAAATATCCA TTAAATCCAATCGGTTGATAATATTTTTTATTTGTGTATCTTTGCTGATTTTCTGTCACCTAGTTCTATTGATTATT GAGAAAGAGGTGTTTAGGTCTCCACATATAATTCTAGATTTGTCTAATTTTCCTTGAATTTTTTCATGAGTTTTGAA ATCTGTTGTAAGACACATATACATTTAGAATTGATGATTTCTTTTTGTGAATTGACCCTCTCACTATGTAATATTCC TTTTTACCCTCTGGGAATTTTATTTGTTCTTAAGTCTGCTTTGTCTTTTATTAATTTTTTTTTACATTTGTGTTTGC ATGGTATATCTTTTTCTATCCGTTTACTTTTAACCTACCTACAGGTATCATTTGAAGTGAATTCTTTATGACGGCAT TTTCTTGTGCATGTTTTTAAAAATCTATTGTGACAGTCTCTATCTTTTCCTTGGCATGGTTAGATCATTTACATTTA ATGTAATTATTAATGTTTCAATTTAGGTCTAACATTTTATTGTTTGTGCCCTCTTTTTTTCCTTTTTCTTCCTTCTT TTGGGCTATTTTTAACTTGTTTTAGTGTTTTGTTTTATTTGTTGTGGTTTTTACTATATCTCTTCATATAGTTTTTC TAGTGACTGTTTTAGGGATTATAATACTTTCTTTCACAGTCTAATTAGAATCACTGTTTTACCTTTTTAAGTGGAAT ATAGAAATCTTATGACTATATAGATCCTTTTAGCTCTCCCATTATGTTATAATATCATGCATTATGTTTACATAAAT TGAAAATCTCATTTCATATGTCATAATTTTTGCTTTTGGCTAATAAACATATCTTGAAAAACTTGAGGAAAATAGCC TGTAATAGTTTTCCATATACTTACATTTTTCTTACTCTGCCTGCATTCCTGATGTTCCAAGCTTCTTTAGAGTAAGT ATATGGATAACTATAACCATCATTTCCCTTTTTTCTAAAGAATTTCCTTTAGCCATTCTAAAGATCAGATCTACTGG CACTGAATTACCTTAGATTTTCCTTTTTATCAGCAAATGTCTCTAACTCTAACTTCATTCCTGAAGGATTCTTAAAC ATCGGATATAGAATCCTGAGTTGTCAGGTTTTTTTCTTTCTTTCAGCACTTCAAAAGTGCTGTTTTACTTCCTCTGG ACTCAGTAGTTTCTGATGAGAATTCCACATTCAAATGACTGTTGCCTTGTACATAATGCATTGTTTGTTTCTAGTGG CTTTCAAGACTTTCTCTGAAGCTTTCAGCAGTTTGATCATGATTTATCTGGGAATGGATTTCTTTGAATGTGTCCTG TTTGCAGTTTTTTGGGCATCTTAAATCTACAGTTGTGTATCTTTTGCCAAATTTGACAAAATTTTAGCTATCGTTTA TTTGAATTTTTTCCCGTACTACACTTTTTTTCCTCTTTTTCTGTAACTCCAGTGACATAAATAGATCTGTTGACCTT GTCTCACGTGTTCTCTTTTTGTCTATTTTAAATACTTTTTTTTCTGTTGTTTGGATGATTTTTTGTTAACTTGGCTT CAGTTTTATTGACTCTTTTCTCGGCATCTCCATTCTACTATTGATCCTATGCAGTACAGTTTTTAATTTTGGTTATT GTATTTTTCAACACTAAATTTTCTACTTTATTATTCTTGATAGCTTCTATTTGTGTTTTTTCATTTGTTTCAAGAGT GTCTTCCCTTGCAGCATTTTTATAACAGATCTCTTAAACTTTTTGTCAGATGATTTCAACTTTTGGGTCATCTCAGC CTTGGCATGAGTTGATTTTTTCCCTGGTGAGTTTATTTTTCTGATTATTTGCATGCTTGGTAATTTTGGATTGTATC CTGGTCATTTTGAATATTATGAGACTCTGGGTTTTATATAAATCCTACAGATAATGTTGATATTTTTGTTTTAGGAG GTAGTCAACCTGGTTGGATTTAGTTAGTTCATAAGTTCTGACCAGCCTTCAATATTTGTTCAATTTTCAAACCCTTT TCCGTCCTCTTAACATCAGTTCTTTTGTGCACCACTCACCAGCCAGTTTGGGAACTGGCAGTGCCGAATTCTTACAG CAGTTCTCAAAGTCTGTGGTTTAGGGTCAGATACACATGTGCATAGGCGGGGAAGTAGACAGCTCACCCGAACAGGT GAGTCCAGAAATCTATCAACAGCTTTTTGGTTTGCTTCCATGAACTCCTTTTCCTCCACAATCTTTTTGGCACTTTT TAGTTCTCAGGGCTCCCTCCTCAGTCCCCTGGACAGACAGCTGGGGCTTTTCCCCACTCCGCTAAACTCTTCCTATT ACTAAACCTCAATCTGGGTCCAAGCATTAGGAAGACAGAGGTAAAGAGCAAAGACATTTCGCCCACCTCTTAGGGGG AGCACACCTCTTCCCATCAGAAAGGGACCATCTCCCCCTCACTGGTTTGGGCTCCTGCGGCCCCTGTTGTCCCCCAC ACCACAGGATTGCTTAGGGAGGAGCTGACGTTCAAGGGCTCAGAGATAACACCAAAAAGGGATGATTCCTCACTCGC CCTGAACATAGTTGTCTCAGCCCATTTTCTGCTGCTATAACAACACTGCCATCTAGGTAATTGATAAAGAATAGGAA TTTATTGGGCTCACAGCTCTGGAGGCTGAGAAGTCCAAGAGCATGACGCCGGCATCTGCTGAGGGCCACCCCGTGGT GGAAGGCATTCCATGGCGAGGAAGCATGCCAGACAGGAGAAAAGGGGCCAGAGTCCCTCAGTAACCAATTCCAGCAG TAACAGCATTCATCCATTCCTGAGGGAGGGGCCCTCATGACCTAATCACATCTTAACACTGTCACAGTGGCAGTTGT TTCCAACACAGGAACACATTGAAACCTTAGCAGCAGGTGATGCTTTTCCTTCCTCAAAGTGGATTAGAGCCGAGCCC CTAGGGGGCTCCCCAGCTGTGCCAGGCTTTGCCTCCAGGGCTCAGGCTGTAGTAAGTTCGGGCTGAGGACAACAGAG GGATAACCCAGGAAGCCCATTCATCTTTTCCATGGTACTTGAAATTCTTATCTTCTTTCCAAGGCTGCCAACTACTA CTGACTCTTCTCAGCCCTCACGTAGCTGCTCTGTGCCTTCTGCCCAGGTTTTATAAAGACATTCAGTAGAAGAAACA AGTATAGAGTGCTTGCTTCATTTTACCTGGAACCAGAATCCCCCTTTCCCTCCATTTGTGTTTCAGGTTAGTTTATT CTGGCTGCTTTTGATGGCCAGGGAGCATTAGGTCAAATAGCTGTGACTTGTGCCAGGGACACCAGCTCCTGGGGATT CCCCTTCCAGGCCAGTGGCTGCTGCACAGGGATGTCCTGCAGGAGCTGAGGACCTGCTCTTTCTTGCTCTCTGTTGC TCTTTGTTGGCCATGGCCACAGCACAGCTACTGAGATGCTCTGCAAGCATGACCCGGCAGGTAGCACCTGTGTGTAT GGGCCTGAGTATGCCCCTGCTAGCTGCAGCCTGGCTAAGGAAAAGTCCCAAAGCCTGGGCTGCTGAAGGATGCCCCT TCCCCCCAGGAAGCCACACGGACCTTGACAGGTCACCACCCATGGAGCTGTGATGGTTGAGGGAAAGGAGACTCCTC CTCAAGAACTTGGAAGCACAATGGCCTGCCTCCCAGGAAAGAGAGCCTCAGTGCTTGTCGGAGCCCTGATCTCAGAG AGGGCCAGCTGCCTGGCCAGCTCTGCTGTTGCCTGTGCTCTGAGCCTGGCCGTGTGCAGTGGACGCACAGGTGTGTT TGCTGAGGTGGCTCGTAGCAGATTATCACCGAGGAGAGATGGACACCCAGGATAAGGAAGTGATGAGTCTGAGCACA GCGGTGGCAGAGCCAGGCTGGTCCCGGTCTGTGTCCTCCTGCCGTGCTCTGGTGTGTGTTGTCTTCCTTATTGGAGC TGGGTTTGGCTGAGTTGGTCCAGGTTCAGGTGTCAGGCCTGCTCAGAGCCTTGAGCTCCACTCCATGGCGTGGCATG AAAACACCTCGCATAAAAACCCAGTGTCTGTGACACAAAACGATTTCTCCCGTGACCAGCATGAGAAAAGGGCATCC AGACCCTTGGCGGAGGATGCACAATGCAGGCAGAGGGTTTAGAAAACAGTCTTCTCTGGGTGAGGCAGTTATGGTGC ACACATTTAAATAGCTGCCCAAGCCGAAATGAAAATAGCCATGTCCTTCAGATGCAGAGACTCCAAGATGCAGGTCC TCTCCCTTGACCCCAGCCACAGAAGCCCAGCCCAAGGTACCCAGCACCACAGGACAGGCAGGAGGAAGGGCGAGTGT GGGAAGGAGACAAGGGAAGTGGAGGGGTAAGAGAGGAACATCCAGAATGTGGCGTTCGGAGGCCGCGTCACAGAGCC CCAAGGTCTCGGGGCTGCCCTCTGGGGAGGCCCATGGTGAAAGAGAGAAGGCTGCTGGGGTTTTTTTTGTTTTGTTT TTTGTTTTTTGAGACGGAGTCTCGCTCTGTCGCCCAGGCTGGAGCGCAGTGGCACAATCTCAGATCACTGCAACCTC TGCCTCCCGGGTTCAAGTGATTCTTCTGCCTCACCCTCCCGAGTAGCTGGGACTGCAGACGAGCACCACCACGCCTG GCTAATTTTTTTATTTTTAGTAGAGATGGGGTTTCACCATATTGGCTAGGATGGTCTTGAACTCCTGACCTCGTGAT CCGCCCGCCTCGGCCTCCCAAAGTGCTGGGATTACAGGCGTGAGCCACCGCGCCTGGACCAGAAGGCTGCTGTTTTA ATGGTTGCCCCTTTGGGCCACCTGAAGTGATTGTTCCAGCTGCTGAGGACTGATGTGGTCACCTCAACCTCAAGAAC TTCTATTTTCCACATGAGTTAATGTCTATGAGTGGATTTCACTCCATTATAGAATATGATGCTTAACAGCAAGGATT GCCCTTCTTCAAGAACGGGTGTCATTCAGCCATGTGATTCTGCACAGGCTAGGGTTCGAAGCACTGTGCTCTTTGAG AGCTGCTTCAACTAATTACTGCATTCGTTCGCTGCAGGGTGAAACAGAGTTTTCAGATACATTTGTAAAATCAGGTG CCTGATTGCTAAATACTACATTGTTTGAGCTATACTTCCTCATATGTTGCCTGGTACGGACCAGTTAAGAAATGGTT TCCCATGAGAAACTGACAATGAAGCTGAACATGTTTGATAAACTCTGTGCTCATCTATGGACATATAATTGTTTTAT TATACGATACTTTTACACTTGGAAAGAAAGGTGCTCCGTAATTTGGCAGCAATTTGTCAGAGCAGCACTCAAGGCTG GCGCTCAGATGAGGCCCTTGGAACCATGCCATTGTTTCTCGGAACTGCTCTGTGCTCCGGTTAGGTTTGCACACACC TCCCTCGTTTCATTCCTACAAGTTTAGTAAAGTTCCAGGCCGTTCTCCCAGAACACCTAACGGAAAGCACAGAAAAC AAAACCACCAACTCTGTGTTCGCCGAGGAAGGCAGGAAATTGGCCTCTGCTTCTTGCGGTATTGACGTGCTGCTTTC ATTGTTTCTTTTCCAAGACAGCTGTCCCCGAAATCTCTTTACCTCCAAGTCAACAGCAGGCTACCTTGCTTTGCTTT GCTTTTTTTTAAAGTTAGGCATAACAACAACAGCAACCATGATAATGATTAACAGTTTGATTTCAAAACGTTTTCTG AAGAAATGACAGTAAACAGTGCATTTGCCCAGGGAAATCCACCACCCCGCCAGCCATCAGCCGGTAATACAACTGCA TGTGCTAATGAACCCAAAGCTGTTAATGTGGTTTCTTCAGTTAAATTGATTCATGAATTTTGAAGAGAACATTATCT AATGAATTTTCTTTGAATAGAAAGCAATTAAAAGGACAAATCAGTCTGATTAACCCCAAAGTCACCCTCCTGCCACA AATGGTGTACAGTTTGAAATTATATCATAAAAAACTAGAGCACATTTGCTATCTTTTTGCCCTCTACTAATCCATGT AAATTAATGAACAACAAAGTTAATTTCTTTGATGACCCTTTTCTAGTATCACCTGGATTATGCTGATGTTTAATTTG CTTAATGTTATAATAAAGACTAAACAGATTTTTTTCCCAGTGAAGTGATTATCGTTCCCTTCAGTTAAGAAGTGATT TTTATTATTTAACACACATGTAAGGGTGTGTTTCCTTTAAGAACCAAATCACACTTTGCCAGCTTGGCACCAAGTGT AATTAGTGTTCTTGTGGACACACTCAGAACATCGTTCCTGCCCGCGGAAGCCTTCCCTTTGTTGCTGCTATTCAGAA ACTCCTGCCCCTGGAGGGGCTGGAGCCCCTGCGATGGAGGACATGGCTCTGTCTCCAGTGCTCCTGAGCTGCATGTC CCCCTTGGAAAGACAATAGGTACTGATTCAGACAGGCCTTCTTCGCGTGCCCCAGTTTCTCTCCCACCGACACCACT CCCAGGTAACAGTTCTGAAACCTGGAGGCAGAGGGAGAGAATGTTCTCCTGCAGTCACCTCCTCATTCCTGCAGCAG GATGCGGCACAAATGCAGTTGGATGTGGTTAGTCAATTAAAATGGGAATCAAGTCATTTTATTTTTGCAGCCCTATT CACTAAAGATGCCTGTAACCAAGGTCACCGCCAGCCCCTGCGTACTGTGGTGCTTGGCACTCCGAGCAGTAGCTGGG GGAATCCTGCACGCGGCTGGAGGTGGACAGCCGGCATAATATCGCAGCCTGGCCTGTGGCTGCTTTTGCCTTCTTTG TCCCAGTTGAAGTTGGAAATTAACCAGCAGTACAAAAGATTTCCAAGCAGGCGGGCAGGCAGGGACTTCTGTGTGTA TTAATTGAGCCATGAGCTCACTTACACACGCCCAGTCGCTAGAAGAGTGGCACACGCAACCCCACGCGGAACACGGG AAGATGGAAAGAAATTGCAAACCTGTGGTGCCAGCTCCAAGGATGCCTTCCAAGGGGCCAGCCTGCATGGTGGCCTG CCACATGCTTGCGTCTCCTCGCCTGGTGACATGGTGGCAGCAGTGCATGGAGGCCGTGTTTTAATTGAGTATAATAC CTCTATTTGTGTAGATGCGTTTCCTGTTTTGGGACTAGTGGCTATTTATATCAGAGCTGCTGAAGCCGTTTGTCCAA ATAACATTATCCTGCATTCTTCCTTTCAGAGTCGTTCACCAGACAGGTGTTATGGAAGCTGCTGAAGGTTGTGAAAT TCGGAGAAGTGATTTCTTACCAGCAATTAGCAGCCCTGGCAGGCAACCCCAAAGCCGCGCGAGCAGTGGGAGGAGCA ATGAGAGGCAATCCTGTGAGTTCTCATGGCGCAAGCATGGCTGTGGGTGGCGGGTGCGTGCAGGTGGCAGGGTGTCA TCTGATCCCACGTGTTTCCTCGGAAGGCAGGCTGTGCTGCTGGGGTGGGCAGAGGGGCGATATCTGTGATGGGGACC ATTATGCCAGATGCCTCCAGAAGAGGCAAGGAGGCTACCCCAGCACTACACTCCTGGAAGCTCCCCCCTACCGGGAG CACATCTCCACTGCATGAGCTCCTGGTTGTCACAGTGAGCAGGAGAGGAGGGAGCCTGGAGGAGTGGCAAGCGGGCA GAGGGTGCCCCACCTGCTCACTCAGGAGGTGGACGTGTTGGGTGCCTGTTTTATGGCCCACACTCTTCTAGGTGCCC AGAGTACAGCAGCTAACAGACAGAAAGGTCTACCTGCGAGGTGCCTGCATCCTAGCAGGATGGCCAAGCAAGGGTGC ACATCCAACCCCATCTCTCTGTGGGCTGCACTCCCCCTGAGGCCCAGGGCTCCCTGGGTCCCAAGAGCTAAAGCACC ACACGTGAGCACACGAGGCAGGAGGCCTTGCAGCATGGGACAAGGGACCTAGACCACACGCACCTCGTGTCTCACCA GGGATGGCGCCCTCCCCAGAGCCCTTCTGTGTCCTCGTCCTTCCCATGCGTCTGCAGTCAGATTTCACTAAACTCCA GGCTCCCAGGCTATGCAGCCTCAGGCCATGCTCCCCAGAGGACAAGGCCCAACCGGGACACAGCATGGGTGGGGCAG CGTGGCCTGTGGTGCCTGGGGGCCACGTGGGTGCCGTTGCTCTCCCATCTCACAGCACCCAGGCCTGGCAGCTGGGC CGCGGGCCATCTGGCATCTGGCATGAGGTGGTAAGTCACAGCATGCCATGTGCGCCCGGCCAAGGCCACACGTGCCT GTGTGTGTGGGGGCAGAAAGGGTGCCCCGTCTGCGTGGCCCATTGGGAGAACCCACACAACCTCAAGAATGCCCCTG GAATATTCACCCAGTGATGGTGTGTGGTCACGTTGGCAGTGTCTTCTCCCGAAAGCTCTGTGAAAGTTTCTAGCATG ATGGAAGCATACCATCTGATCAGGGAGCACAAATATGATCACTCCATCATGGGATGGGAAATTACTAAATCAGATAG ACCTCACTTTTTGAAAAATTGCTGTGGCTTTACTGTCTATTTCCAAAACTACTTATATCCCTGCTCACAGGGGAGAG GCCGGCGTGTTCATACCACAGCCTGCTGCTCCTGCTGCCCGCTGCAGCAGCCTGGGGGCAGAGCCTCCGTTTCTAGG AGATGACAGTTACGTAGGCTTTTGCACAAAACTGCATTGTTAGTCCTTAAAAAGCAACTCTGGTAGGTTTGTAGTGG ATTGCATTGTCGTAATGTACACCCTAATCTAGGCGAGCTTTCTACCCTATTAAAATATCCCAAATATCCCAGGAGTG AGATACCACAGGGACAGCCACCATCCCCACCTCCTCCATCTCTTCCTGTCCTCAGTTTTGGTGACAGTGGCTGGTGG GCGCAATTGACCCTTCCTTTTGCACCTGTTGCACATTAATTGCAATGTCTTCAGCTACATTGTCATAAAACATCTGC ACCACGCAGTAGGGATCTGATTTTTACGGCGCGTTGGGGGATATTTCCGAATCAAATTTCTGAGGATGGTTTTTGAC ATTTCCCAATAAAACTTAACCTACATGCCCCTCTGGAAGGCTGCTGTCTTCTCCCCGGTTGTGAGTCAATTTTGAGG TGTTCAACCTGATGAAAAGGCAGCTCAGATGCTGTTGTGTTGTGGCTCCGTGTAGGGCCCATGAGAACTGTGGATGC GAATGGGGTTTTGGGGGTGCTCTTCCTGCATCAGTGCAGTGACTGTTTCAGGCAGTGTCTACTCTTTGTGCCCCCCG AGGAGAGTAAGTCCTTGTCTTCTCCCCACTGAGGTGGATTCTGCTCCCTCCGTGCACACACATGAAGATGGGAGTGG CCCCGTCAGCGGGCGCCGACTGCTCGCTCCCTTGTGTGTGCAGCTCCCCTTTTGCTGTAAGGACAAGCTCCACTAAC TGACATGTGCAGCGTGGCAGCACCCTCCCCCACCAGCCTTTGGTGATGCCAGCAGTTTCTCACTCTGCAGTGGAGAT GCCGTCGCTGGTTAGCAATCCTGCTTGTGGCGATATTCGGAGACATTTATTGTTCTGCAAACCAGCGTGACTCTCTT CTCCTGCTATTAATCTCTATGAGTGTCTCTGTTCCAGTATGGCCTCAGTCACTGAAATGTATAAAGTAGTATCACAT GGGATGTAAAACCCTAATGTCTGTAAAGTCCACACATCGGCACCCTGTCCCATGGCAAAGCCAAGACTGGAGGTGAG CACAGCATCAGGCAGGAGGGTTCTACAGGCACCAAGTCTCGCCCTTCTCGGCGTCCACATGCCTCCTGTGCCGAGGG CAGCCAGGCGGTGGTGGCTTCTCGTGGCCTTTCTGTGGGCTCCACCGTGTGCAGTGACCTTCAGCCTGAGGAGGGGG CTGCAGCAGGACCTGTGGCTACGGTACCCACAGACGCCTCAAATTATAGCACAGATATGCCAGAGATATTCCTGCAA GAAGCCTTCAGCTACGTAGTTTACTATGGTTCAGGTTGACAGCCTCCCAAGTTCCCATTAGAAACCTCATTAGAATG GGCTAGGAGGATGAAAAGGTGACCCAGGGACTTGCTAGAGGAAGGGCGCTGGCCAGCAGTGAGCCATTGCTGCTGTC AGTGTCCTCAGGAATGTTTCCCTTTGGCGCTGACAGCAGTGTCCCAGCCGGAAGGCTGCGCACATCCACTCCAGCCC AGACGGTGCGTCGCTGAGACTCGCTCATTGAAAACATCCCTTATATTCAGGATGAATAGAGTGATTGTGCATTTAAG GGAGGAGAGTGAAAAATGGACTTGGAATGTTAGAAAACATCGTAAAGCTCATTGTGCATTAATTCCTTAGGTGGCTT GCAATAAACAGGAGCACGTGCTGGTTCATATTACTGCATTTGCTGTCGGCATGAGTTTCTCTTCCCCAACCGGAGTA AGGGATGGCTTTTGCCATGCGGAGCTTTCCTCTATCCTAGGAGCTTGATTTCTCCAGAATCCTCCAGGAAGGACTCA CCAGCCGGTGTAAGGTGGTTGGTCAGCATTCCTGGAAGGCTGTCTCCCCAACTCAGCGGGAGAGGATGGTCAGTTTC CCAGTATATTGAGTAGAAAACTCCTCACGACCCACCCCCTCCACAAAGCACCCTTCCTCTTCATCCTCAGACAGAAT TTGCTAAATGCAGTTCAAAGGCAATCGATACATTGCACAGAGAGTCTCCCCACCTTTTTCTGAACAAATGCATTCCT GAGGAGTTAGTTTATTCACGGATTGCCATGAGCGTTAATGACCCCCCGAGGCTTGTTCTGCCCGTCACCCGGATGGC TGCTGATAAAGACTTAGCATCATGTTCTAAGTGTCAAGGTTGTCAGTGGAGCTGAACCATTTGACCAGAAGAAAAAA ACGCACAGGGAGCTATTGATTACAGCTTCTCGGTGTCACTTGTTCACCAGGATTTAGGTATTGATGAGGCTGCGAGT GAAAACAAGCTCTTTCCCTTGTGGGCTGCTGCCCAGAAGAACAAAGGCGAAGTTGGAAAAAATATTCAGCCGGTACC AGAATCAGAGGGATTCCATCATTGACTGAAAGGCCCCTTCATGTTATTTTTGTTGGAAACAAATCTATGGGTTTGGA AGTTTTATAGCGTTTGAGAAAGGGGCTGCATAAGTGATGGATTCACGTATTGGATGAATTAGATTTCACAGCATGAA GATTTCAATGCAGCTGTGATATTATGAGCTTCTTGGTTATTGATAAAAATTACATTTTATGCAGCACATGCTTTAAA GGAGAGGGCATAGAGGGTACAGATCCTTTGAAAATGTCCTTAGGGCCCCAATAAATTTCAGCAGGTTTCATTATCTT AATAGTCTGAAACCTGCCACCAAAATTCACACAAACAGAAGCAGCGTTCCCTATTTCTAACGGTAAAAATAGTGCCT ATTTGGTAAGTCACCTCTTCCCCATCACAGCATGCTAAGGTGAGGACCGGTCACAGCTGTCGGGGTCATTGTTATTC CGTGCCATCTGTCCTGGCCTCTGCATTGATGTACAGGAAGAGGGAGAGGGAGACATGTTTATTTTACTCATTTTTAT AATTTCATAGCTCATTGAGAGGGAGAGAAGAAAAACCAAAGCTCTGCTGTGAAATCTGCTATTAGGTAGGAAAATTG AATTGGTGCCTAAATGTTTATAAAACTAGATTGAATTAAATGCCTACCTCTTTATCAGTCAAAAACGTGAATTGTGT ATTATCTAATAATGCCCTCACAGCAATTATTAATGGGACTGTTGTATTTGATAAAAATCATCAATCAGGGTAAATAT TCCATCCTGGAGATTTCCTGTGACTCCTTCTGGAAGACTGGGATTCTCAGCTGTTCCAGAACTTGGCAGTTACTACA CCTCACTCTAGTAAACTCAATGTTAAAAACAGTCAGCCCTCCGCCACCTGCATGTTCCCATGTCATAGTCGTCAGAA GCCTCTTAGACTGCGTCATTGGTGCACGTTACCAAACTCATCGTCAAATTAGGATGAGCTGTGTGATGTGCCGGAAG CCAGAGTCCCACCTTGCAGCCATGTCATCTGCTGGCACCGGCTGTCACCCCACAAGGTCCCACCTCAACAGTCCACG TCAAGGAGGAGAAGTGTGGTGCGTGGAGCCATGCAGGAGAGTGGTTAGAGCCCCTTCTTTGCCACTTAACAGCTTTA TGAACTTGGGCACGAGACTCCATTCGGCCGGAGGCTGAGACAGGCCTGGTGGGGCCTTGCGGGGTGATGGGGGCCAT AGATGGGGTCGAAGATGCAGCAGCATCAGGCATGGGCACACCCGCTCCCAGGGGCTTTAAGCACCTTTTCCAAGGGG GGCTGGGGCTTCCCCCACCTCCCTCGGGGACGTGTGTAGCTGGGCCCCACAGAGGGCCACACCCACCAGGTGAAATC TGTGGCAGGTACATGTGTGGCAGTGACACAAGCTGCCTGCACCAAAAGCCTCAGGGGATGCAAAAGTAGCAGTCACC TCCCCAGGAAGCAGAGGTGCCCAAGAAATAGAGCTTGGAGATGCTGGGCACACGCCCCCAGCTGCTTCCTCTAAAAG CTCACCTGACCAGTAGCTGGTTGCTTTGTCCAGTTCACTGGCTTTGGAGGGTGGAGCGTGGGGAAGCTGGGCATGGG CAGAAATCATTTCTCTGTTGAGCTGGGGATACGCTCAGCTGGAGAGAAAAAGAAGAGTGGAGTCGGAGTGATACCCG GGGGTACCCGGAGTGATACCCGGAGGTAGCCTGGACACGCCCCAACCTTGGGATCCTGCAGAAACAGCTGTTTGTTC TCATCAGGGCAGGATGCTGGGAGCAGGGCAGCCATGTGTGAGGGCCCAGGGAGAGGCCCCGGGTCTGCAGCATCCGT GTCTGGGCCATCTAGGTTGCGGGGATGAATGTGCTGCCCTCCAGCTCAGGAGGAAGGGAGGAAGGGGGGCCAGGCAG TAAGCGATCGTTAGGCATCTCCCATGTGCCTTCTACGTGTCGTGCCAGCTGAGAACCTTCCAGAGCGAGCTTGGTGC CCTTCCCCATTCTTCTGGAGCCTGCCCTGGCTCCTGTGGTCATGCTCATTGGCAGTTTACCTTCTTCACGGAGCAGC CACTGTGATGCTTTGCACGATGATGAGGCATGTCCCCCCACACGTCCCCCGAGATGTCCTGGCCCCTTAGTGGCTGC CCACCCATACACCTGCTCCCCAGGCGCCAGGAGCTACTTAGGGATGAAGGCTGTTGCCTGGGAGGCTCTCCAGCCAA TTCTTTCTTTAGAAATCTGTGCAAGACTTCCCCACATGCTCCTGGGAAGCCATTCTGGAGCAGATCTTCACAGTAAC TGCTTGTTGGGAAGCTTAAGCAAATGGCCCTGGAGCCCCCAGAGCCCCCAGAGTCCCCTGCGGTGGCCATGCAGGCT GTGTGGCCATCCCTGTCTGCCCAGGCAGAGGGGGACAACCAGAGAGATGCCCATATGTTACTGCCTTGTCTTTCACC TTTCTGAGCCAGCTGAGAAATGGATAGCAGACCTCAAGATTTTAGCTTTATTAGGGTAAAAGCGAGATTGAAGGCAG GGGGTGGGGGGCCAGTGGCATGGCCATGGTGGCTTCCCAGTAATTCCCACAAGAATTACATTCGCCATCACTGTCCC CTCTGGCTAAGCTCTGGCCTCCCCAGCCAAGGCAGGGGCTGACCATCTCAGAAGAGATCAGGGCGCCCCGAACCCTG GGGCAGGCAGGTCACCTGCAGGAGCCCTAACAGTGCTAAGCTGGGCATACCCCTCTCTTCCTCCCTTGTCGGTGGTC AGTGACTCATTCTCCCTTGTGGGTGTGAGAGTGGAGGAGGCCAGCACCTACATGGAAACCAGAGGCAGGGGCCACAG CACCTGCCCCCACCCACCACAGGTGGCTCTCAGAGCTTCAGCCAGACAGTGGACCCATGGAGCTCCTCCTGTCTACA CAGCTCGGGCAACGCCAAAGGACCAAAGCAGAGTCTCCCTGAGGACTGCACTGAAGGAGGCGCCCGCCCCTCTGGAG GGAACAGGGATTCCCTTCCCCTGGTGGGACTGGCCATAAAGAAGCAGTTGGTAGGCAGGATCCGGGGCCTGTGTATG GCTGCGTGGTTGTGGTCCTCACTCTGGATCAGCCCCTATCAGCACAGGGCATCCTGAGGCAGGGGTTGGCCAAGTCC TCCTGTCAAGGGCCAGAGAGTAAATATGTTAGGCTTGGCAAAGCAGCCACAGCCAGTATGGAGAAAAAGGCATGGCT GCGTTCCAGTAAAGCTTCATTTGCAAAAGCAGGTGGTGGGCTGCGTGCGCCCACAGCCACAGTTGGCCGGCCATGGG CTGGAGAGGCCCGTGCAGGTACGGTCTTCTCTGATGCGTCCTGTGATGCCTGCGTGGTACCTGGGGAGTGTTTGAGA GCGTCACATACCACCAGAACTCGGAAACGTAGTTTTCAGTTTTGTAAGCGAACGTTGTATATTACTCACCGGATGAA ATTAAGTTTTCTCCTCCCAAATCGTGGGCCTAAAGCAATTCTCTGAGGATGCCCCACGTCACCTCAGGACACTCGTC CCTCCCCCAACCCGTGCTGAGACCCCCAGGGACTCAAGGGCCTCAGGGGAGGGGAAGTCCATGCTGAGACATAGCTG ACACCCACCCATGCCAACAGCCTGCCCCTGGCACAGGCCCCTGCTTGGTGGGCACAGGACTCCTGTCAGTCAGGGCC TTGGCCTTGACCCCAAAGACCTCGTTGTCCAGATCCCTGACTGACAGTGGCTGCCCCCCTGTCTTCCAGGTCCCCAT CCTCATCCCGTGCCACAGAGTGGTCTGCAGCAGCGGAGCCGTGGGCAACTACTCCGGAGGACTGGCCGTGAAGGAAT GGCTTCTGGCCCATGAAGGCCACCGGTTGGGGAAGCCAGGCTTGGGAGGGAGCTCAGGTCTGGCAGGGGCCTGGCTC AAGGGAGCGGGAGCTACCTCGGGCTCCCCGCCTGCTGGCCGAAACTGAGTATGTGCAGTAGGATGGATGTTTGAGCG ACACACACGTGTAACACTGCATCGGATGCGGGGCGTGGAGGCACCGCTGTATTAAAGGAAGTGGCAGTGTCCTGGGA ACAAGCGTGTCTGCCCTTTCTGTTTCCATATTTTACAGCAGGATGAGTTCAGACGCCCGCGGTCCTGCACACATTTG TTTCCTTCTCTAACGCTGCCCTTGCTCTATTTTTCATGTCCATTAAAACAGGCCAAGTGAGTGTGGAAGGCCTGGCT CATGTTGGGCCACAGCCCAGGATGGGGCAGTCTGGCACCCTCAGGCCACAGACGGCTGCCATAGCCGCTGTCCAGGG CCAGCTAAGGCCCATCCCAGGCCGTCCACACTAGAAAGCTGGCCCTGCCCCATCCCCACCATGCCTCCCTTCCTGGC TGTGTCCATGGCTGTGATGGCATTCTCCACTCAGCAGTTCCTAGCATCCCACACCCAGGTCTCACTGAAAGAAAGGG GAACAGGCCATGGCAGTCAGTGCTTACAGAGGTTTTCTAGCTTTGTTTCCTACCTGCCAAAAAGGCTAATGCAAGAT GCATCCACTTATGGTCTGTATCAGGTGAATAGAAAATAACCATTTGCCAAGATTTGGAAAGACAGAGAGCTCTCTGG ACACGGGTTCGATGGAGCTTGCACCTCTCTGAGAGCTGAAGTCAAGTCTCACCCTCTCTCCATACTCCCTGGCCTAG TGTGCTCTGGGCAGCAGGTAGGAAGGGACAGGGACCTCTCATCTCAAGGGCTGCCCCAAAGGGCCCTCAGGGACCAT GGGCCTGTTAGGACCCCAGCACCCCAGTGTCGATCCTGGAAGTCAAAGGTCACGACTGTGGGGGGCCAGCTCATGTC ACTGATGGCAGCAGGCAGGGTTTCCAGCATGGGCGGTGCGCTGCTGTCTGATACTTCATTATTGTGTTGTCATCCTA GCTGTATAATTAACTATATCCAAAAGCCGGAAGGGAAACCTGTAGCTCTGCATATGGAGAAATAAAACAGAGCATAT GATGTATAGAGAAAATGAACTGCACCCAGCTGTGCACTAGAGCGCCATGTTCTTACCCAGCATTCCTTGCCACGGTG TGAACCACAGCAGGGAAGCTCGCATGGTTCTACACGTCACCGAGGCCTGGATGAAAATGTGGCCTCACGATGTGTAT GGTTGGGACCCGCCTTCTATTTCATTTTATCTTAACACCAACGAAAGAAAACCGATTTCAGACGTTTTTCGCTGATT TATGATTTATTATATCCCTTTCTGGCATCAGCCAAAATCTCGGTTTTTCCTATGACATGAAGTGATGTGACTCTTAC CCCGTTGTCTTAACTCTTGGTATTTTTGAAGCAGGATAAGTCACACAGCGGGTCACGTAGACACGTGCTGTTTTGTG GGACAGGCCCTGGGCCTCCTTGCTCCTGGCAGGCCTCAGGTGCCGCACGCCGCGTCACCGTCAGGACTCGCAGGCTG TCTGGTCATTTTTGACGTTGCTGAAGGTGAGTGGGAAAAGCTGGGTGCACTCTGCCCCAAAGGTGGTGCCCTGTGTC CCCCACAGATGATGGCCCTCCCCAGTGATGGCCGGTCACCAGGCACGGCTTTCCCCTCTGCATGAACAGAACAGAAG CTTCTGGCTCTCCAAGGCAGCAGCTTGGGGCCTGGTGGCCCGGGTGTGCGAGCCGCGAGTGCTTTCCAACAGAGGGC ACCAGAGCCGTGCGGTGTGCTGTGGAGACCCCAGCACCACTTCCCCTGCTGGAGGATGGATTTGAAGGAGGGTGGCT GGGCGGCACCCCGATGGCTCCCTGTGCTCCCTGAGACCCTACAGGCCGCCCTCTGCAGCCACACCTGGACCGGCAGG CATGCACGCTGCAGGCAGGCTGCCTCCGCTGTTTGGGTCCCATGAACGATGGAAGCCTCCCAGTGAGATCTTCAGGT CTCTCATCAGGCACAGGCTCTGAGGGAGGGTCCTGGGGCCACATGACTGCAGCCAGCGAGGGCCGAGCCCCCAGAAT CCAGGTCAGCTCCTGGTGGCAGGGGACCTGCCCGTCAAAGCCGTCCAAGGTGAGCCAGCAAATCCGTGTGGCCTGGG AGCACAGGTGGGCCTGCTCGGACCATGCCCTTGGTGCAGTACAAAACCATGTTCTGCTTCCTCTCCGAGGGTTTCCG CGGCCCTGTCAGAACAGGCTTGATAGATGCCCGGAGGTTCCCTCTGACAGCCGCAGCAGCAGGGCTCAAACCGGCGG GGCCGTGTACCGCTCCAAGGACAAGCTCTGCCGAGGCCGACATGAAAAGCCAGCACGCGGGTCGCCTAGAGCCGCTC TGCCTCCAAGCTGCAGCGCCGTTCGTGAAAATACAGAGGGAGAGGGAAGGCACAAAGAAATACAAAACTATTTTCCA AATAAAAAGAAATGTCGAAACAAGAAAGCTGTGTTCTTCAGCCTTGGCCCTGGGTCCCCCTCACCCTGTCGCATTTC CCAGCACTGTGATGCTTTGAAGGCCGACACCGAGACCCGCCGTGCACGACAGCCACCTCGCAGCCACTCAGACCGGC GGACCACGCTCCCCTGTTTCCAGAGAGATGAGAGAGTCAGAAGTAATACTTGTTTTTGCAGAACCACACTTGTGGCA AAATAAGTTGCCTTTGGCCACGCAGGAGACTGAGTTTTGTGAGGGGTTGTCCTTCTCCCAGTCTCTCTTCCCGCTGG GAACAGAAGTGTCCAGTAAACCCACCTGCATTCCACGTGTCCTGTGGCCGCCTCCAGTGGTGAGTCTGTGAGGCCAG AGAAGAGAAGCTATGACCGTGCAAACATGCATGTTATGGTGTTGGCTGATGCGGAATCAGGAAGCTTGGAGGAGCTC TTGCATGAGGAATTGCAGAACTTAGGGGCATGAATTTGCACTTCCAGATGTGAAGGTTGCAGGCACCGGGCGTTGCA GAGGAAGTGGGCAAGCTCACCTTGTAGAGAACTTACTTGGGGTTTGTAATTTGTATACACTTCTTACCCAGCAGAAA CAGCTTACTGAACACCCCTCGGGTGGCTGGATGATGTGCTAGCAACTGAAACCATCTCTGCGACTTAAGCTTCTCTG GTGCGTCGCGCCCCACGTGCTTCTCCAGAATCCCGTTTGGAGGGGCCAAGCAAGTCCAATATAGATAGGTTTATACT GAAGCCATCAGTTTTGTTTCTTACATCGTTTGTAAACAGCAGTATTATCCTGGAACTTGTTTCCTACAGAACTGTAA GATAAGGATAAAATCCCATCCACCTGAATTGCTGAGAAACGTAAGAGGTGGTGTGACCCGCTGGAGCCCTGTGGAGT GGCGGACTCTGGGGAGTAGCTGGTCAGTGAGATTCTCGCAGCAATGTGAGGCATCCTCCTCACAGAAATGCATTTAC GGATGTTGCTGTATCCCCTGACCTGGCCTACAAGGACAGAACAGCCCTGGGGGCCACGTCCCCTCCCGGTCTCATCT GCCGCTTGCTCACCTTGGCTGTCCATGCACCCGTAGCTGTGACCATGGGCGGTGGCGCCAGCTCGGACTTCACCCCG TTGGAGCGGGCAGGATGTCACACTGAGCTAATGTGAGATTGTTCATGAAGCTACAGATCTTAGTGCTACTTGGGCTT CTCACAGCACAGAGGAGGATTCTGAAGTGAGAGATAAATTGGCTTCATGTATGAGCAAGGGGGAAAGTGTTTTATTT TACTAGGAAGATTTTACTGAAAGACCAATCCCGAAACGGCCCTTGCTTCGGCCACAGCTGCTGGGATGTCCTCGGGC CTCCCCTGTTTGTCTGCATGTCTTCTTGTTGCCATCGGGGCAGTGGAGTCCCCGGAAACAGTCCAAGGCCCTGGGGT GGGGGATTTAAATTTTTGGCTTCCCTCAGACCTCAGACCGTGTGGGTTTTTTTTTCTTTCTTTCTTTCCTGTTCATG GGCATTTGATTAAAAGTTTGTGTTTAAAGAGCTGAACTTCACATCAGTTTTACTTATCAGTTTGAAAGACATTGAGA TCTAAACACGGCTCTGCCCAAGACCTCTGCCAAGCAGCGCCGTCTGTCTGTCTGTCTGTCTGAAGGGACGGGAAGGT GGAGAAACTGCAGGTACTGACCTGTCTCTAAGCCACTGAGGGCAGCCATCCAGTGTCCTCGGGACCCCATGGCTCCA GCTGCTGACAGATGTGAAACGTCTGGCCTCCATCACCCGGGTCGCCCCGACAAGGATCTGTAATGGGGGCAGGGGTG GGGGTGGATGAGAAAGTAGGAAGTGGATGCTGGGCAGCGAGGTGTGAACTGACGGCAGAGCCCAGCCCGACAGCACC TTTGATTGATGAAAGGACATTGTTAACAAGCCTTTAAAGTTTCTCATTAATGTTTTTTTCGTTGGAAAAAAAAGCCA CCAGAAAAAAATTCCACATGGACAGTAATTAAGATCGATCCCGTACAGTATGGTATCAATGTTTGGGAGCCTTTAGG AAAAAATTATCCACCCTCCTATTAAAGACTTATTTTCAAGCTGCCATTTTCCAGGCTGTTGAAAGTCCCTGGCAGTG TGGGTACCTGGGCAGTCAGGCACGTTCCGCAGATGTGATGTGGTCGGCCTCACAGGTAATGTTTCTACTGTGGCCTG AGGATGGAGAGTGGAGCCCCTGAGCTTTAGTTGTCACGGACTCTGATGCAGTCAGGCAAGGAAGGGTGGGGAAGGAT CTGATGGATCTGCTGCCATGGAGAAGCTGGAGTTCTTCTCAGCTCCGAGTGAGCCCCAAGGAGTGCGTCTGAGCAGA GTGCACGTGGTGCCCATGGTTCTCACCAGAGATGCTCAAGCCCTTGGGCGAGGGGGCTGAGCCAGCCCCGACATTGT ACACACAGCACCACACACGGGCATACCCAACGGTTCTTTACAGGTCTTCGAGTTACACGTCACTGCTGTTTGGATCC CAAACACCAGCGGAACGCATGATTCCAGGTTGATTTGTTGACTCACATTAAGGAAACCCAGCTGGAGAAAGTATCAG GGTCCTAACGTTAAGCAACCTGCGATATTATGCAAAAAGGATTGCTTTTGTGTGTTGTGATTGCCATGGCTACGAGA AGAAATGGATGCACGTGTGAGACTTGGCTGGTGACAAGGAAGCATGTATGGACATGGAATCAGGTCCTGAGCAAGCA GGCGGTCCTCCAAGGCGGTGGAATTGGCCCTGGATCCCTGCCCAAAGGCCGCTCCGCTCTGGGCCTTGGTGGGAAAG CTCTGCCCTCTAGGCTGGCCTCTGAGAGGGCTTTGGTGTGACCTTCCTGTCACTGCCCACTTTCTAACCAGACAAGG AGGGTGTCTGTACCTGGCGCTAACCCAGGGTTTGGGGGACCGTCCTGCAGTCCCCGTCGGACAGAGAAGGCTTCGTT TCTCAGGAGCTCCAGGCTTTGATTCACTCCCACGATGTGTTTCTTTTTGGGGTAAAACAGTCCCCACGGGGAGTTAA ATTTTAGGAAGCCATGAGATCCTTCCAGATGAAAGGCATCATGTAAGTGTATGATACTGTTACTGAGCAAAAGCCCC AGTGCTCCCAGGTGTGGCAACGAGACACCTGCACTCACCGGGCACCACCCAGCCGGGGGGGGCGCTCCCCGGGACTG GCAGGTTCTCTAGGTGTGGCAGTGAGACATCACTCACCGGGCACCACCCAGCCGGAGTGGGTGCTCTCCGGGACTGG CAGGTTCTCCAGGTGCCTCCTTCCTCTTGCAGCTTTGCTCACTTTGTCAATTTTCAGGAAGGGGAATTTTAAATGTC CCCCCAGTCCTGAAGAGATTGAGCAGCATTTTCAGGAGGAGGCCTCACCATCATCATTGCCACACTGGCCATAAACC AGAGACTCTGCTGGAGAAACCCTCTGCAGACCTTGGAGGCCTCACACATCCCAGTTACGGGCAGCGCAGCTGTCCCC TCCGGAGAAGATTCCTCTGCAATGGGAA

GenBank Accession No. Z84721 GATCACGCCATTGCACTCCACCCTGGGCGACAGAGCGACGAGACCCCGTATCAAAAAAAAAAAAAAGAAAGAAAGAA AGAAAAAAGAAAAAAAAAAGGCCGGGCGCGGTGGCTCACGCCTGTAATCCCAGCACTTTGGGAGGCCGAGGCGGGTG AATCACGAGGTCAGGAGTTCGAGACCATCCTGGCCAACATGGTGAAACCCCGTCTCTACAAAAAAAAAAAAAAAAAT TAGCCGGGCGTGGTGGCGGGCGCCTGTAATCCCAGCTACTCGGGAGGCTGAGACAGGAAAATCGCTTGAACCCGGGA GGCGGAGCTTGCGGTGAGCCGAGATTGCGCCACTGCACTACAGCCTAGGCGACAGAGCGAGACTCCGTCTCAAAAAA AAAAAAAAAAAAAAAAAACACTTGGAAGCCGACAGGAGATCTTTGAGACCTTGGGCGAGGCAGTGACACTAAAGGCA GGAGCGACTACAGAAGAATAAATTAAACTTCATCAGATTAAAAACTTTACTGCGGCCGGGCGCGGTGGCTCACGCCT GAAATCCCAGCACTTTGGGAGGCCGAGGTGGGCAGATCATGAGATCAGGAGATCTAGACCATCCTGGCCAACATGGT AAAACCCCGTCTCTCTACTAAAAATACAAAAATTAGCTGGGTTTGGCGGCGCCTGCTTCTAATCCCAGCTACTCGGG AGGCTGAGGCAGGAGAATCGCTTGAAGCCGGGAGGCGGAGGTTGCAGTGAGCCGAGATCGTGCCACTGAACTCTGGC CTGGCGACAGAGCGAGACTCCATCTCAAAACAAAACAAAAACTTCGGTGCTTTAAAGGACACCATCAAGAAAATTAA AAGTCCACCCACAGAACGGGAGAAAATATTTGTAAGTTACATATCTGATAAGGGAATTGTATCTAGAATGGAGGAAA CTTACAACTCAACAATAAAAAGACAATTGAAAAATGCACAAAGGATATGAATATTTTTCCAGTGCATTATGCAAATG GCCAATAAGCACCAGAAGATGCTCAGCTCAACTGGTAGAGGCTTACGCCTGTGACCCCAGCGCTGAGAGGCCAGGAA CTCCAGACCAGCCTGGGCAAAACAGAAATTAAAAATGCTCAACATTATTAGGCATTAGGGAGATGCAAATCAAAACT ACAAATAGATGCCACATCACACCTCCTACGATGGCTGTAATCAAAAAGACAAGCGTCAGCAGGGGTGTGGAGAAACG GGAATCTCTCTCCTGCTGGTGGGAATGTAAGAGGCTACACTCGCTATGGAAAACAGGCTGGCAGTTCCTGAAAGGTT AGAGTTAACACAACACTCGGCAAATCCCCCTTTTAGATATATAGCCAAGAGAAATGAAAGCATATGTCCACACAAAA ACATGTGTGTTCTTAGTAATATTATTCATAATAGCCCAAAGTGGAAGCAATCCTAGGGTATATCAATTGATGAATGG GTGAATATGGTATAGTTTGTTTAAGGGAATACTATTCAGCCATAAAAAGGAATGAAGTACGGCACATGAATCCATCT TGAAGACACACTAATATATGATTCCATTTATATAAGATGCCCAGAATAGGCAAATCCATAGAGACAGAATGATTAGT GGCTGCCTAGGGCTTCCAGGGGGTCAGGGGAAATATGGAGCGATTCATGGGTTTTTTGAAGGGGAGTGATGAAAATG TTCTAACGTTGACTGTGGTAATGGTTGGACAGCTCTGAGAACGCGAATACACTAAAAGACATGGAAGTGCCGGGCGC AGTGGCTCATGCCTGTAATCCCAGCGCTTTGGGAGGCCAAGGCAGGCGGATCGCGAGGTCAGGAGATCGAGACCATC CTGGCTAAGACAGTGAAACCCCGTGTCTACTAAAAATACAAAAAATTAGCTGGACATGGTGCGGGCGCCTGTAGTCC CAGATACTCAGGAGGCTGAGGCAGGAGAATGGTGTGAACCCGGGAGGCGGAGCTTGCAGTGAGCCAAGATCGCACCA TTGCACTCCAGCCTGGGCGACAGAGCGAGACTCCATCTCAAAAACAAAAAAAAGATATGGAAGTGTACACTTGAAGT GGATAAGCTTTATGGTATGCAAATTGGTATGGTATGGTAAATTATATCTCAATGAAGTTGTTTTTTAAAAAATCACC CCACCTACCCTATCCCAGGCTTCCCCAGGAGGTAACTAAAGGTAATGAGCTTCTTTGGCTGCTTCCAGAACTTTCCC AAGCACATCAAATGCATCAGAACCTAACCACTTGACTGAGGGATGAGCATTTTCACTGTTGCAAGTAACCCTCTTGC ACCAACACTGACACTAATGTGTATTTTGCAGAACAAATTTGTGGATTGGCCTCACCAGGGTGAAGGGTACGTGCATT TGAAATGGCTCAACAGTACCAACAGGTGCGTTTTCTTGCACAGGGCTGCATAACATTTTTTTTTTTTTTTTGAGACA GAGTCTCGCTCTATCACCCAGGCTGGAGGGCAGTGGCACAATCTCAGTTCACTGCAAGCTCCACCTACCAGGTTCAC ATCATTCTCCTGCCTCAGCCTCCCAAGTAGCTGGGACTACAGGTGCCCGCCACCACACCAGGCTAATTTTTTTTTTT TTTTTGAGATGGAGTCTTGCTCTGTCGCCCAGGCTGGAGTGCAGTGGCACGATCTCAGCTCACTGCAAGCTCCACCT CCCAGGTTCACACCATTCTCCTGCCTCAGCCTCCCCAGTAGCTGAGACTACAGGCGCCCGCCACCACGTCCGGCTAA TTTTTTTGTATTTTTAGTAGAGACGGGGTTTCACCGCGTTAGCCAGGATGGTCTCGATCTCCTGACCTCTTGATCCA CCCGCCTCGGCCTCTCAAAGTGCTGGGATTACAGGCGTGAGCCACCGTGCCCGGCCTGCATAACATTTTTTTTTTTC CTGAAATTCCCAGAAAGGAAAATGGTGTCTTGTTCTATGTTGCATTTCTTTGATTGAGAGGGAGAGCTGCATCACTT AATTATTTGCAGAGAATTGCTTTTCTTGTTTTCTTTACAGGTGGTCTGTTCTTGGATGGTCTGGCTGTGTTCTTTCT GAGGAATACATAACCTCTGCTACACATTTTGCAAGGCTTTATCCCCGTTGTCCATGTTTTGATTTTATGTATAATCA AAAGGTTTGTGAGTTCTCCCGCACTTCCCAGGAGTGCCTCTGGGATGGAAATGAGACTGCAGGAGCAGGGCTTGAGG CTGGAGGGGTGAGATGGGACAGATGGGGGTGGGGGAACCCAGGGCAGTGGCCGGTGGTGGTAATGGAGGCCTCCTCA CAGGGACCCTCACAGCGACCATGCGAATGGAGCAGGACTGTGACTCAGGTCTCGCTCTTCTGACCTAATCGTGCTGC TGCCCCAATGGGCAGAACCTTGGGGCTCCAGACTGGACATCTCTGGGCTCAAAGGATCCCACTGTTCCCCCGGTTAC CCTCTCAGGGTTGGCCTCCTGCCAGTAACCCTGGCACTCATTGTTCATTCTTCTGACTATCGTCAGTCATAATGAGA GCTCGAACTGGTGAAAGTGCAGGGAGCTCACCATGACCCCAGCCCACAGAGGTCCTGGGTGCGTCCCTGCCCTCGAA GCAGCACTCTGGATCCCAGCGCCACCCTCATGTCCATGTTTGCACCTCATTGGCTGTGACAGAAATGAGACATCATT GTCACACGCTGGCCTGAGGGTCAGTGGGCCTTGCTTTGGACCTCAGTTTCCCCACCAGTAACAGGGTTCAGAGCAGA TGGTCCCTGAGTGAGTCCCAGCTCTAAGTTCTCCCAGGGTCTCCTGGACAATGAAGCACCAGGGCCAACCTCCATTT GCTACAGGGGACATCCTCAGGCTCTTCTCTGCTAAGACCCCACACCTCCAAGTCTCCTCATTTTACCTTTAAATAGC TGTTTCATGACCTGCTTTTTTGACGGTAAGTAGATTTTTGGAAACTGAAACCCCTGACCCTTCCTCCCAGCCTGGGC CTGCCCTTGGCAGGATAGGAGGCCTTATCGGTCCTGCCACTTGGTCTGGGCCTCAAAGGGCCACCGCCATCTGCAGG AGGGCCGGGTGGGGTTCACAGACGCTATCTGGGACTTGCCTGGACACCTCCACCTTCTCAGCTGAGTGTTGCTGCCC CACCAGGGAGAACCACTCACACACAGTAGTAATAGAAATAATTTAAAATTCATGCTGCAAGTTCCTGAGCGCCCTCC CAACACTGAGGTGGGGGCTAGTCTAATCCCCATCCTAGAGGTGAAAACAGTGAAACTAGGACTCACAAGGCAAATTA GCCTGTTCAGGGTCACCGAGGGTCCACTCTCATGGGAGAGTTTGCAGATGCCCAATCCGGCATTCTGCTGAGTGTCC AGTGGCTTGTAAGTGGCCAGACACCCTTTGAGCTCAGCCTCAGCTGCTCAGGCACAGAACGTGCCTGGAGCTTGGAA TTCAGGCCAGAAACCACCAGTGGACACCAGCATTCCACACTCACTGCACAGGCTGGGGCTCAAACCAAGGCCCAGGG ACAGGAAGGGACAAGCCCCAGCCCCAGCCGGACTCCCAGCCCACACAAACCATCAGGGCTTGTTTCCTGCTCCATGG AAGCCTCAGACATGTTTCATAACCTCCTGGAGCCTCCGTTTCCTTATCTTTCCAATGTAATGATGCCCATGTGCAGT GGCTCACGCCTGTAATCCCAAGCACTTTAGGAGGCCGAGGTGGGTGGATCACTGGAGCTCAGGAGTTTGAGGCCAGC CTGGGCAACATGGCAAAACGCCATCTCTACTAAAAACACAAATATTACCCAGGCATAGTGGCACATGCCTATAGTCC CAGCTACTCAGGAGGCTGAGGTGGGAGGATCACCTGAGCTTGGGAAGTTGAGCCTGCAGTGAGCCAAGATTGTCACA CTGCACTCTAGCCTGGAGGACAGAGTAAGAAGACCCTGTAACAAAACAAAACATAACAAAACAAACAAACAAAAAAC CCAACTAATGACAATAAAATAAACCCTCCCTCACAGGGTGGTTGTGAGGATAAAGCACCCAGAATGAAGAGTGTTGC TGCCATGTGCAGAACTTAGAAAGTGCTCAACAGATGCCAGCCAAACAGACATGGACTCCCCTCAACACAGTCAACCC AAGGTTGACTGTCACCAAACGCAAAAGACCACACTGTAAAGCTTTTAGAAATGTGGTCTAGTGGCCGGGCACTGTGG CTCATGCCTGTAATCTCAGCACTTTGGAAGGCTGAGGCGGGCGGATCACAGGGTCAGGAGTTCGAGACCAGCCTGAC CACCTGACCAACGTGGTAAAACCCCGTCTCTACTAAAGATTCAAAAAATTAGCCGGGTGTAGTGCTACGTGCCTGTA ATCCCAGCTGCTCGGGAGGCTGAGGCAGGAGAATCGCTTGAACCCAGGAGGCGGAGGTACAGTGAGCTGAGATCGCG CCATTGCACTCCAGCCTGGGAGACAGAGAGAGACTCCGTCTCAAAAAAAAAAAAAAAAAAAAAGTTAGCCGGGTGGT AGTGGCATGTACCTGTAATCCCAGCTACTTGGGAGGCTGAGGTAGGAGAATCGCTTGAGCCTGGGAGGTAGAGGGTT GCGGTGAGCCAAGATGGCGCCACTGCACTCCAATCTGGGCGAGACACTGAGACCCTGTCTCAAAAAAAAAAAAAAAA TGTGGTCTAGGAGACTCTCTTCACTTTGAGATAAAATTTGCATCACGTAAAGATAACCATTTTAACGAGAGCAAGTC AACGGCATTCAGCACATTCAGAGTGTTGTGCAACAACCACTTCTCCCTGGTTCCAGGACATTTTCATCGCCTCAGAT GGAAACGCCCTCCTCACGGAGGCATCTCTCCCGGCCTTTGTCCTCCCCGGCCCTGACAACCACTAATCTACTTTCTG CTGGGATTTGCCCATTCTGGATGTTTCCTAAAAATGGCTTATCTAAGCCCCACAGTTTCATGCAGCACGTAGCCTCT GGTGTGTGACGTCCTTCACTTGGTGTAATGGTTCGAGGCTTGTCCATGTCGTAGCCTGGGTCAGAACTTCATTTTCA TGGCTGAATAATATCTCACGGTGTGGAAATATCACAGTTTGCTTATCTGTTCATCCAGTGATGGACATTTGGGTTGT TTCTACCTTTTGGCTATTGGGAATGGAAGGGATAACATTTTTTAATTGGATTTTTAAAGTCACTAGTTTGACTGCAT TAAAATTACAAACTTTTGTTTAACGAGAATATCACTAAGATACAGAGTTGGGGAGATCTAACACATAAAAGTGACAA AGGAATTATATCCAGAATATTTTTGAAATTTCTACAAATCAGTGACTGGCAACACAGTGGGAAAGTGGCCAAGACTA AAATACTTTAATAAAGAGGAAACCGAAATGGCCAGTAAATATGGGCTCAACCTCACTAATTATCAGGAAAATGTAAA TTAAGACCACAAGAGAAACCACTACACACTCACCAAAAATCACACACCCAATAAAAAGGTAATTTTTTTTTTTTTTT GAGATGAAGTCTCACTCTATTGCCCAGGCTGGAGTACAATGGCGCGATCTTGGCTCACTGCAACCTCCGCCTCCTGG GTTCAAGCGATTCTCCTGCCTCAGCCTCCTGAGTACCTGGGATTACAGGCGCACACCACCACACCCAGCTAATTTTG CATTTTTAAGTAGAGACGGGGTTTCACCATGTGGGCAAGGCTAGTCTCGAACTCCTGACCTCGTGATCTGCCCGCCT TGGCCTCCCAAAGTGCTGAGATTACAGGCATCAGCCACTGTGCCCGGCCTAAAAAAGGCTAAAATTTAAGAAGACCA GGAGTTTGACTGCTATGGTTGGAATGTTTGTCTCCTCTAAAACTCTTGTTGAAACTTAATCCCCAGTGTGGCAGCGT TGAGAGGTGGGGCCTTTGGGGTAAGGAGGTTGGATCATGAGGGTCCTCCCCCAAGGAATGGATTAATGAGTTGTCAT GGGAGTGTGGCTGGTGGCTTTATAAGAAGAGAGACCTGGCCGGGCACGGTGGCTGACACCTGTAATCCCAGCACTTT GTGAGGCCGAGATGGGCGGATCACAAGGTCAGGGGATCGAGACCATCCTGGCTAACACAGTGAAACCCTGTCTCTAC TAAAAAAAAAATGCAAAAAAATTAGCCGGGCGTGGTGGCGGGCACCTGTAGTCCCAGCTACTAGGAAGGCTGAGGCA GGAGAATGGCGTGAACCTGGGAGGCGGAGCTTGCAGTGAGCCGAGATCGCGCCACTGCCCTCCAGCCTGGGCGACAG AGCAAGACTCTGTCTCAAAAAAAAAAAGAAGAGAGATCTGAGGTGGCACACAAGCATGCTCAGCCCACACGACCTGC GATTAATACTCTGTGCCACTTTGGGACTCTGCACGAGTCCCCACTGGGCTCGAAACTTCTCAGCCTCCGTAACTATA GGAAATAAATTCCTTTTAAAATAAATTCCACAGTCTCAGGTATTCTATTATAAGCAACAGAAAATGGAGTACTACAC CGATCATATCAAATGTTTAGAAGGATTTGGAGCAAGGAGAATGCTCGCACACCACTAGGGAAAACATAAGTTGGTTA ACCACTGTGAAAAAGTTTGGCATTCTTTACTAAAGTTGAAAATCTATATGCCCTATGACCCAGCAACTTTACTCCTA GGTATGTATGTACAAAATAGAATTTCAGGCATGTGGGTACCAGGTGACATGTAAAGGAATGTTTATTGCAGCATTAT TCATAATAGCCAAGAACTAAACAACACAAAGTTCCAGCCCCAGTACAATGAATAAACTGTGGTATATTCCTACAAGG AAATATTAATAGATACAGCAATGAAAATGAACACATATAACATGGCTGGTAAATCTGACATGAGAGAGTGAAAGAAG ATGGACATTCAGTGTGCAGACAGTTGGATTAAAAATATTTTTTTAAAGGCCAGGCTTGGTGGCTCACATCTATAATC CTAGCACTTACAGAGGCCAAGGCGGGCAGATCACCTGAGGTCAGGAGTTCAGGACCAGCCTGGCTAACACAGTGAAA CCCCATCTCTACTAGAAAATACAAAAATTAGCCAGGTGTGGTGGTGCATGCCTGTAGTCCCAACTACTCGGGAGGCT GAGGCAGGAGAATCACTTGAACCTAGGAGGCGGAGGTTGCAGTGAGCCAAGATCGCATCACTGTACTCCATCCTGGG TGACAGAGCAAGACTGCGTCTCGAAAATAAATAGATAAATAAATAAATAACCAACAGGCCGGGAGCAGTGGCTCATG CCTGTAATCCCAGCACTTTGGGAGGCTGAGGTGGGCAGATCACGAGGTCAGGAGATCAAGACCATCCTGGCTAACAC AGTGAAACCCTGTCTCTACTGAAAATACAAAAAAATTAGCCGGGCATGGTGGCGGGCGCCTGTAGTCCCAGCTACTC AGGAGGCTGAGGCAGGAGAATGGCATGAACCCGGGAGGTGGAGCTTGCAGTGAGCCGAGATCATGCCACTGCACTCC AGCCTGAGCGACAGAGCGAGACTCCATCTCAAAAAAATAATAATTAAAAATAAATAAATTAAATAAATAAATAACAG ATTGCATAAAGTGGCTCATGCCTGTAATCCAAGCACTTTGGGAGGCCAAGGCAGAAGGATCACTTGAGCCCAGGAGT TCAGGACAAGCCTGAGCAACATGGTGAAACCCCACCTCTACAAAAAAAAAAAAAAAATTAGCTGGGCATGGTGGCAT GTGCCTGTGATCCCAGCTACTTGGGAGGCTGAGGCAGGAGGATCACTTAAGCCTGGGAGGTCGAGGCTGCAATGAGC TATGATCGTACCACTGCACTCCAGCCTGGGCAATAGAGCAAGACCCTGTCTCAAAACAAATAAACAAAAGCCAGACA GACACAAATGAGAGCATTCTGTATCGTTTCATTTCTATGAAGGTGAAAAGCAGGCAAAAACAACCAAAGTGCTTGCA GATGCATATCTGAGTAGTTAAAAACTTACTGAAAAGCAGGCCTGGCTCACGCCTTTAATCCCAGCACTTTGGGAAGC GGGCGGATCACGAGGTCAGGAGATCGAGACCATCCTGGCTAACACGGTGAAACCCCGTCTCTACTAAAAATATAAAA AATTAGCCAGGTATGGTGGCTAGTGCCTGTGGTCCCAGCTACTCGAGAGGCTGAGGCAGGAGAATGGCATGAATCCG GGAGGTGGAGCTTGCAGTGAGCTAAGATCGTGCAACTGCACTCCAGCCTGGGCAGCAGAGCGAGACTCCCTCTCAAA AAAAAAAAAACTTACTGAAAAGCAAGAAGTCAGGTGGAGGTTACCTTTGGGGAGGATTGGGGTGCTGTCCGCTTTCT AATAATTCGTTAAACTATAGTCTACATCTTGTGCTATATTTCACAATGGAAAAACAGAAAAGAGCTCCTGCCCATAA CGCTGCTTTGCAGGTTTGGAAATTTCAGATTCAATTCCTCTCCTTGCGGGGGCCAAGGATGGGAAGAGCAGGTGGTT CCAGTAGGGAAAGAGGAGGCCCTGGGGCCTCAAAATGGCTAAGGACCATTCCTCAGCGTGGGTGGCACCTACCCTGG AAACAGGACTCTACTTCCTCCTCTGTTAGGGGGCAGAGCAGCCCTGCAGTGCCTTCTGGGCACAGGTCCTCACTCTG CAGCTGGAGGAATTCTCCCAGGCACTGAGAGCCCTTCACGGCCCAAATGCCCCGTGCGCTCGGCCTCTGGACTTGCC TTCCCTGCTCTGTATATCTCCCTCCGCCTGACCCTCAGCCTCCTCCATCACTCACTGTCTTCTCTGCCAGTCTATTC ATCTGTCTCTGTCCCTCTCTCTGCCACCTTCTCTCCTATTGAGAAGCCGAAACCTCAGGCACAGACCCACATCCCCT CCTCATGGGCCCATGTGCCCAAGGTGCCCCTAGGTGCCAGGCTGAGATGAACCAGGAGTGTCCTTCTGAACCCAGCA ACAGCGAAGGGTGACCAGGGAGGGCCAGTTCATCTCGGTCTGAAAGAAGCCCCAGATGAGCAAAGGATACACTGGCC TCCTGCGGTCAGCAGCACTTCCCAGGACAGTGAGCAAGACAGGGGTAAGGCCAGAGTGGGTGGGCACACCCATGGGA GAGAGGAGCCGCTGTGAAATGTGCACGAGGAACAGACCAGCAAGGAGGATCCACGCAGTGCTAGAAGGGAGTTCCTG GAAGCCTGGTGGAGAGCCCCTCCCATCTGCTAAGCCCGGAGGGCATCAAAGGCTGCTGCTGCCCTCAACCCCTGACA ATCTCATCATCTCATATCTCAGGCATGGAAGAATGAGGGCCATTACACGAGTAAAACATCAAGTACACTCCAGCCTG GATGACAGGGCCAGGCTCCATCTCAAAAAAAAATGCCTGTGGTCAAAGCTCTCCTGACAGGGGAAAACAAAACAAAA CAAACTTCTCCTTAAAGAAAACATTTGCCTTTGACTGCATCATAATTCCAGCAGGATTTTGTGCAGATAACTCTTTG GCTAACTCTAAAATTAATACAGAAAGGTAAAGAAATTAGAATAGCCAAAGAAATTTTGAAAAGGAAGAATAAAGCGA GAGGAATCACATTCCTCAATTTTTAACAGCTCTATTGAGATAAAATTCACATACCATACGGTTCACCCATTTAAAGT GTATAATTCAGGCCGGGCGCGGTGGCTCACGCCTGTAATCCCAGCACTTTGGGAGGCTGAAGCGGGCAGATCACCTG AGGTCGGGAATTCGAGACCAGTCTGACCAACATGGAGAAACCCCGTCTCTACTAAAAATACAAAATTAGCCAGGCGT GGTGGCTCATGCCTGTACTCCCAGCTACTCGGAAGACTGAGGCAAGAGAATTGCTTGAACCCGGGAGACGGAGGTTG CCATGAGCCGAGATCGCGCCACCACACCCAGCTGCCATTTTTTAATTGATTACTTGTCTATTTATTACTGAGTTGTA AGATATTTTGGGCCAAGCACGGTGGCTAACGCCTGTAATCCCAGCACTTTAGGAGGCTATGGTGGGCAAATCACTTG AGGTCAGGAGTTCGAGACCAGGCTGGCCAACATGGCAAAACACCATCTCTACTAAAAATACAAAAAAATTAGCCAGG TGTGGCCAGGCGTGGTGACTCACGCCTGTAATCCCAGCACTTTGGGAGGCCAAGGCGGGTGGATCACCTGAGGTCGG GGGCTCAAGACCAGCCTGACCAACATGGAGAAACCCCGACTCCGCTAAAAATACAAAATTAGCCGGGTGTGGTGGTG CATGCCTGTAATCCCAGCTACTCACGAAGCTGAGGCAGGAGAATGGCTTGAGCCCAGGAGGCAGAGGTTGTGGTGAG CTGAGATCATGCCATTGTACTCCAGCCTGGGCGACAAGAGCGAAATTCTGTCACAAAAAAAAAAAAACCATTAGCCA GCCATGGTGATGCACACCCGTGGTCCCAGCTACTCAGGAGGCTGAGGTATGAGAATTGCTTGAACCCAGGAGGCAGA GGTTGCAGCGAGCCAGGATTACGCCGCTGCACTCCAGTCTGGGTGACAGAGCAAGACTCTGTCTAAAAAAAAAACAA AAACAAAAAAGATATTTTGTATGTGTTTGGATAACTTCCCTATCAGATATATGATTTGCAAATATGTTTCTCTCATT CTGTGAGACATCATTCAATTTTAAGACATCACAGAGCTATGTTAATCAAGGCACTGTGGCTGTGGTAAAGGATAGAC ACACAGAACAGAACAGAGAGCCCAGAAATGGACCCGCAAACCTATGCCCCATTCATTTTTTACAAATAAGTGCGAGA AGCCAACTGAATAGAAAGCGTATAGCTTTTTCAAAAAACAGTGCTGGAACAATTGGACATCTGTAGGCAAAAAAACA AACAAGCAAACAGAAGAATCTGGACCTGCCCTTCACACCTCAGACAAAAGTCATCTCAAAATGGATTGTAGATCTCA ATATAAACATAAACTATACAACTTTAGAAGAAAATATAGGTGAAACTCTTTGTGTTCTGTGGTTAGGCAGACAGTTC CTAGGCATGGCACTAAGTAAGATTCATTTAAAATTTTTTGACAAATTGGACTTTATTAAAACTTTTGCTCTACAAAA GACAATATTAAGAGAATGAACTAACAAGCTACAAACTAAGAGAAAACATTTGCAAATTGCATATCTGACAAGGGATT GCTTCCAGACGATACACAGAATTCTAAAAATTCATCCTTAAGAGAATAAACCACCCAATTTTTAAATGGGCAAAACA GGCCAGGCGTGGTGGTGCACGCCTGTAATCCTAGCACTTTGGGAGGCCGAGGCAGGCGGATCACAAGGTCAGGAGAT TGAGACCATCCTAGCTAACACGGTGAAACCCTGTCTCTACTAAAAATACAAAAAATTAGCCAGGCATGGTGGCAGGT GCCTGTAGTCCCAGCTACTCGGGAGGCTGAGGCAGGAGAATGGCGTGAACCTGGGAGGCGGAGCTTGCAGTGAGTGG AGATCGCACCACTGCGCTCCAGCCTGGGCAACAGAGCGAGACTCCGTCTCAAAAAAAAGACAAAATACTTGAAAAGA TATTGGCTAGGCGCGCTGGCTCATGCCTGTAATCCCAGCACTTTGGGAGGCCAAGGCGGGTGGATCACAAGGTCAGG AGTTCAAGCAGCCTGGCCAAGATGGTGAAACCCCGTCTCTACTAAAAAAAAAAAAAAAAAAAAAAAAAAAATTGGCC GGGCACAGTGGCTCATGCCTGTAATCCCAGCACTTTGGGAGGCTGAGGCAGGTGGATCAGGAGTCAGGAGATCGAGA CCATCCTGGCCAACATGGTGAAACCCCATCTCTATGAAAATACAAAAATTAGCCAGAGATGATGCCGGGTGCCTGTA ATCCCAGCTACTCATGAGGCTGAGGCAGAAGAATCACTTGAACCAGGGAGTCAGAGGTTGCAGTGAGCTGAGATCGC ACCACTGCACTCCACCCTGGGCGACAAATCGAGATTCCATCTCAAAAAAAGAAAAAAAAATTAAAAGGAATATTTGC CTCATTATGTTACAATAACTAATATGGAAAGCAATATTGCAATGCCTATTAGCACATGACATTAGGTGAATTCTCCT TTGTCCCCGGACCTGCTGCCTCCTCCTGCTTGTCAGGGGACAGATCCAGTACATCTCCCCTCAGCGCTGGGTGGACC TAACCCTTGCTTTCTTGGAGGAAACCCAGGAATCCAGAGACAAAGTGGAAGGGTACTGGCATGTGGTTGGGCAGGGC TGCCTGAGGTCGGTGTCAGCCGACCGTGGGGCTTGGTCCCAGGAGGCTGCTTACTGGGCCCTGCTCCTCTGGTTTCC CCCAAGTCGTGATTCTGAAATGAATAAGGACGGTGCAGAACTGGACTACAAATGCAGGAGTGACTTCCTGGGAGGGT GGGGCCCCTATCTCTCCTAGACTCTGTGGTCAGACTCTGGCCAACACCCCCTGTAAGGCCACAGGAGAGGAACAGGA GTGATAGCCCCCAAACCCCAGTCCCACCAGGCCCTGAGGGCCCCTTTGTCACTGGATCTGATAAGAAACACCACCCC TGCAGCCCCCTCCCCTCACCTGACCAATGGCCACAGCCTGGCTGGGCCCAGCTCCCTGTATATAAGGGGACCCTGGG GGCTGAGCACTACCAAGGCCAGTCCTGAGCAGGCCCAACTCCAGTGCAGCTGCCCACCCTGCCGCCATGTCTCTGAC CAAGACTGAGAGGACCATCATTGTGTCCATGTGGGCCAAGATCTCCACGCAGGCCGACACCATCGGCACCGAGACTC TGGAGAGGTGAGTGTCAGACGGGACTGCCAGAGGGACTGGGTGGGAGGCCAGGTATGTGAGTGGGGACAGTGGGGAG GGGGCGGTGGGGAGGGGACAGTGGGGAGGGGACCATGGAGAGGAGACAGTGGGGAGGGCACTGTGGGGAGAGGACAG TGAGGAGGGGACCTTGGGGAGGGGACAGTGAGGAGGGAACCGTGGAGAGGGGACAGTGAGGAAGGGACAGTGAGGAC AGATAGCGTTCCCTCTCAGTGAGGAGAGCAGGGTAAGGAGGGAACGATTAGGAGTTGCACAACCATCTGGGCTCGCT GAGACCTGGGCAGGCACAGGCCCAGGTTCTGACAAGCAGAGGGTGAAAGGTTTCGTTCTAGGCCTGAAGGGCCTTAC AGGGCAGCCAGGGCACTACAGCCTCTAAAGTCCCAGCATCTGGGATCAGGGCACTGTCCCAGCTTCAAATTCCCAGC ATCTGATCCCCTGGGAGGGGCCAGGGAGCTTTTCCTTCCCTGGAACGCTGCTGGGAGGTCATGAGCCTGCAGAAGGG GTGGCGGGCAACCCAGTCTGGGGCTGGGAGGGAGGTCCTGTGGCCAGAGGAGACGGTGGAGGGGCTGGGGGCACCAG GCGTGCTGGAGGCGGAGGGCGGGAGATTTGGGGACCAGGCTGCACAGAACCCGTCGGAAGCAGGGCGATCAGCCGGG AGCTGCAGAGGCCTGGGGGGCCTCTAGCCCAGGGCAGCCTGGGAGGGGCAGCTGCCTGGGCACCCGGGCCCCGCGAG GAGGGGCTGGGGCCTGCTGCGGGGTCGCAGATGTGTCCCGGTGCTCGGAGAGGGCCGCAGGGCGCGTGGGCCGTGGC GGGAGGCCGCGCTGCTGGGAGCTCACGGCCCCCGCCCCCCGTCCCAGGCTCTTCCTCAGCCACCCGCAGACCAAGAC CTACTTCCCGCACTTCGACCTGCACCCGGGGTCCGCGCAGTTGCGCGCGCACGGCTCCAAGGTGGTGGCCGCCGTGG GCGACGCGGTGAAGAGCATCGACGACATCGGCGGCGCCCTGTCCAAGCTGAGCGAGCTGCACGCCTACATCCTGCGC GTGGACCCGGTCAACTTCAAGGTGCGCGGGGCGCGGTGCGGGGGGGGCGGGACGGGGCGGGGCGCGGTGCGGGCGGG GCGGGGCGGGGCGGGGGGGGAGGGGCGGGGAGGGGGGGGGTCGCGGGGCGGATGCGGGGGTCGCCGGGGGGGGCCC GGGCTAGGCCCCGCCCCCTCACTGAGCCGCCCCCGCCCCCAGCTCCTGTCCCACTGCCTGCTGGTCACCCTGGCCGC GCGCTTCCCCGCCGACTTCACGGCCGAGGCCCACGCCGCCTGGGACAAGTTCCTATCGGTCGTATCCTCTGTCCTGA CCGAGAAGTACCGCTGAGCGCCGCCTCCGGGACCCCCAGGACAGGCTGCGGCCCCTCCCCCGTCCTGGAGGTTCCCC AGCCCCACTTACCGCGTAATGCGCCAATAAACCAATGAACGAAGCAGCGTCCACCTGGTCTCTGTTGTCCGTGGGCG GCGGGCGCTTGGGGAGGCGGAGCGGGAGGAGGGCGCCCCGGCTGTCTCGGGGCCACTGCTGGGCCGCAGGGATCCTT GCACCGACCCCAGGGTCTCTAAGAGGCAGAGGGATGTGCAGCTCCCGGGGGGGGAGCGGGGGTCACTCGGGACCCAG GCGTGGTGGAGAAGGGGTGCAGTTAGGCCTTTGCGGAGGGGGGAGCAGTGCTGGCGCCCACCCGCCGCGGCTCTCCC TGGGACCTCCGTGGTCTTCCTTCTTTATTTCTCCCGAATGTGTACTATTTCCTGATTTCAGAACGATCAGGACGAAG AGGGGAGGGATGGGCGTCTGCGCTCACTCATTCCTTCTTCCATTCCTCAATGAAACATTTACTGGGCATAAGACAGC CTAGGCATGTTTCTAGGCTATGGATACCGCAGCTGAAATAAAGAAAGCCCTCTGCCCCGTGGGGCTGACAATCTAGT GGGGGATACAGACGTGATGAAGACAGTCAGATCACAGTTCACAGAAATGAGACAGGAAAAGAGGCTGAGCCTCACTC ATAAGAGAAACGCAAGTTAAACTACACAAAAATAAAAAACCTCACTGAGATCCATGTCTCACCTCCCTGATAGGCAA AAATCCAAGAGTTTGATCAGACTGCAGGCGCCCCTCCTCCACTGGGCACCCCTCATCCAGGGCAGAGGGAACCAGCC CGGGGCGCAAGTCCACCGGGGCATCTCATTTGCTAAAGACCTGAAAACCCAGGTGTCCATCATCAGGACTAACTGGA AAAACCAAGGGTATCCGCACCATGGAGAGCTCGACTGAAAAAAAAAAATGAGGATAATTGGATAATTTCTTTTTTTT TTTTTTTTTTTTTCAGACGGAGTCTCGCTCTGTCGCCCAGGCTGGAGTGCAGTGGTGCGATCCCGGCTCACTGCAAG CTCCGCCTCCTGGTTTCAAGCGATTCTCCTGCCTCAGCCTCCCGAGTAGCTGGGTCTACAGGCGCCCGCCACCACGG CTGGCTAATTTTTTGTATTTTTAGTAGAGACGGGGTTTCACCGTGTTAGCCAGGATGGTCTCGATCTCCTGACCTCG TGATCCACCCGCCTCGGCCTCCCAAAGTGCTGGGATTACAGGTGTGAGCCACCGCGCCCGACCTAAAATGAGGATAA TTTCTAATAATGAAAATAAAGAGGTTAGAATGGTGTGTATACAATGGTGGAACAGAGGAGAAACACGAATATGTGTG TGCACATATATGTGAGCTTATGCATAACTATGTATGAGGCTGCGTGTGGACATGTGTGTTTGTGCACAACCATGTAT GTGCCCGCATGTGCTTATTTCTGCAAAAATAAACCATGGCAGGACAAACCGGAAATGAATACAAATAATAAGGTGGG TGGGGATGGAGGGGAAGGTGGAAGGAAGCTCCTGCAAGTCTGACTCTCTACATAGTTTTGACCTTTGATTTGTGTAA ATATTTTACATTATCAAAAATAAATTCAGGCTGGGCATGGTGGCTCATACCTGTAGTCCTAGCACTTTGGGAGTCCA AGGGGAGAGGATTGCTTGAGGCCAGGAGTTGAAGGCCACCCTGGCCAACATAGAGAGACCCTGTCTTTAAAAAAAAT TACAAAATTAAGGCCGGGCGCGGTGGCTCACGCCTGTAATCCCAGCACTGTGGGAGGCCGAGGTGGGCGGATCACGA GGTCAGGAGATTGAGACCGTCCTGGCTAACACGGTGAAACCCCGTCTCTACTAAAAAGTAGAAGAAATTAGCCGGGT GTGGTGGCGGGTGCCTGTAGTCCCAGCTACTTGGGAGGCTGAGGCAGGAGAATGGTGTGAACCCGGGAGGCGGAGCT TGCAGTGAGCCAGGTTCAAGCCACTGCCCTTCAGCCTAGGTGATAGAGTGAGACTCCTTCTCAAAAAAAAAAAAAAA ATTACAAAATTAATAAGATTAAAATAAAAAGAGGGGCCTTGCCAGTGGCTCAAGCCTCTAATCCTACCACTTGGGAG GCCAAGGCTGGAGGATCCCTTGATGCCAAGAGTCGGAGGCCAGCCTAGGTAACACAGCAGGACCTCGTCTCAAAAAG ATTAAAAAATTAACTGGGCATGGTAGCCTCCAAATTGGGGGTTAGCCTGGGAGGTTTGCCCAGGAAGGAATTCAAGG GCAAGCTGGTGGTGTTACACAGCAACTCTGATTGATATCGAAGCCACAGCAGACAGCAGGAGCAGAACACTGCTCCT TACAGAGCAGGGGTACCCCATAGGCTGTGTGCACAGGAGAGCAACTCAGAGGCACTGCTGCACTCATCTTTATACCC ACTTTTCATTATATGCAAATTAAGGGAAAGTTATGCACAAATTTCTAGGATGAGTGTGGTAACTTCTGGGTGGTCCA GTCACTGCCATGGAAAGGGATGGTAAACTCCCATGGCACACTGGTGGGTGTGTCTTATGGAAAGCTGCTTCTGCCCT ACTTGTTTTAGCTGGTCCTCAGTTTGGTCCGGTGTCCGAGCCCAACATCCGGAGTACATGCAGAGTCCCACCTCCTA CGTCACACCTGCAGTTCCAGCTACTCAGGAGGCTGAGGCTGGAGGATTGCTGGAGCCCAGATGTTGAAGGCTACAGT GAGCTATGATTGTGCCACCGCACTTCAGCCTGAGCAACACAGCAATACTCTCTCTCTAAAAAAGCAAAGCACACAAA CAAAAAGAGTGACTGGGTGCAGTGGCTCACACTTGGAATCTTAGCACTTTGGGAGGCCAAGGTGGGATGGTCACTTG AGCCTGGGAGTTCAAGACCAGCCTAGGCAACATAGCAAGACTTTATCTCTACTAAAATATATATATATTTTTTAATT AGCTGGACATGGTGGTGCACCTGCAGTCCCAGCTACTTGGGAGGCTGAGTTGGGGGTGGAGGGGAGTATCACTTGAG CCCAGAAGTTCCAGGCTGTAGTAAGCTATGATTGCACCACTGCACTCCAGCCTGGGCAACAGAGAGAGACCTTATCT ATATTTAAAAAAAAAAAAAAAAAGAGAGAGAAAATTGAAAACTCCTAATTGAAAACCCCCAAATTGAAAACTAACTT AAATAAATGAGCCAATGTAAGAATGTGGTGATATAATAATCAGAAAAAAGGATTGTTCCAGGTGACCTCTGAACACA GAACCTCGGCTATGACCGAAAGAACTCCAAAGACACTCTAACACTCCGTGGTTTATTGTTCCTCATAACATATATAA AATAATTTCATAAGCTTTTATTTTGAAACATATTCAGATTATGAAGAAATAAAAACACCCTGCAAGAATAAGACAAA GATGGAGAAGGAAGGATGACTGCTGGTGGGTTTGGGGCTTTTGGAGGGTGATGGAAACCTTCTAAAATTGATTATGG TGATGGTCGCACAATTATGTGAACACATTAAAAATTATTGAAATGGGCCGGGGGTGGTGGCTCACCCCTGTAATCCC AGCACTTTGGGAGGCCAACGCGGGCAGATTACCTGAGCTCAGGAGTTCCAGACTAACCTGGCCAACATGGTGAAACC CCCGTCCCTACTAAAAATGCAAAAATTAGCCACGCATGGTGGCACATGCCTGTAATCCCAGCTACTGGGGAGGCTGA GGCAGGAGAATTGCTTGAACCCAGGAGACAGAGGTTGCAGTGAGCCGAGATTGTGCCACTGAACTCCAGCTTGGCCG ACAGAGTGAGACTCTGTCTCAAAAAAAAAAAAAATTATTGAAATGTACACATTAAGTGGGTGAATTTTATCTCAATA AAACTGTTAAATAAAATAACAAGAATATGAAAAACTCTTGAATACTACTCATCCAGACTCTCCAGCTGTTAACATTC TACCACATCGGCTTGCTCTCTCTTGCCCCCACTTGCTCTTTCTCTCGGAGCCCTTGGAGAGGGGTATGCAAATATCC GTACTCTAAATATCCTCCATATACTGTGTATTTCCTAAAATCAACAAGGACATTAGGCTGCACAGCCAGAGAACAAC CATCAAAATCAGGTTAATATTGATCCAAATCCATCTATCAACAGAAGCAACATCAAGTTCAAGACCCTTTTGAAAGC AATGATACCAGCCATTTACTCCATCCCTAAAGGACTGAGGGTGCTGCGAATTTAACCGTATCAATGCAGTCTTTTTG ATGTTATTTACTGAAGGAAATGGATGTTCTTTAAAATATGTATTTATTTATTTTTCTTTTTTGAGACGGAATCTTGT TCTGTCGCCCAGGCTGGAGGGCAGTGGGACAATCTTGGTTCACTGCAACCTCTGCCTCCTGGGTTCAAGAGGTTCTC CTGCCTCAGCCTCCCGAGTAGCTGGGATTACAGGCGCGAACCACCACGCCCGGTTAATTTTGGTATTTTTAGTAGAG GCGGGGTTTTACCATGTTGGCCAGGCTGGTCTCAAACTCCTGACATGGTAGCCTGTAATCCCAGCTACTCGGGAGGC TGAGGCAGGAGAATCGCTTGAACCCAGGAGGTGGGGTTGCAGTGAGCCAAGATCGTGCCATTGCACTCCAGCCTGGG AGACAGAGCGAGACTCCATCAAAAAAAAAAAAAAAAAAAATTCCTGAAGCTCCTCTTGAGCTTACATTCTAGTGGAC TGTAAACAGAAACATTTTTTTTTCCTGTGGATAAAGAAAAGCAGGGCAAGTAGGGGCTTAGACAGAGGAGGGGAGGA TTCAGATTTTAAATGGGTTGGCCACTGTAGGTCTATTAACGTGGTGACATTTGAGGGAGTGGCAATACTAGGGAAGG GGCTTCAGGGGAGTGGCCAGGAGCTAGGGATAGAGGGAGGGAGGACAGGAGGCCTTGTCTGTCTTTTCCTCCATATG TAAGTTTCAGGAGTGAGTGGGGGGTGTCGAGGGTGCTGTGCTCTCCGGCCTGAGCCTCAGGAAGGAAGGGCAGTAGT CAGGGATGCCAGGGAAGGACAGTGGAGTAGGCTTTGTGGGGAACTTCACGGTTCCATTGTTGAGATGATTTGCTGGA GACACACAGATGAGGACATCAAATACATCCCTGGATCAGGCCCTGGGGCCTGAGTCCGGAAGAGAGGTCTGTATGGA CACACCCATCAATGGGAGCACCAGGACACAGATGGAGGCTAATGTCATGTTGTAGACAGGATGGGTGCTGAGCTGCC ACACCCACATTATTAGAAAATAACAGCACAGGCTTGGGGTGGAGGCGGGACACAAGACTAGCCAGAAGGAGAAAGAA AGGTGAAAAGCTGTTGGTGCAAGGAAGCTCTTGGTATTTCCAATGGCTTGGGCACAGGCTGTGAGGGTGCCTGGGAC GGCTTGTGGGGCACAGGCTGCAAGAGGTGCCCAGGACGGCTTGTGGGGCACAGGTTGTGAGAGGTGCCCTGGACGGC TTGTGGGGCACAGGCTGTGAGAGGTGCCCAGGACGGCTTGTGGGGCACAGGCTGTGAGGGTGCCCGGGACGGCTTGT GGGGCACAGGTTGTGAGAGGTGCCCGGGACGGCTTGTGGGGCACAGGTTTCAGAGGTGCCCGGGACGGCTTGTGGGG CACAGGTTGTGAGAGGTGCCCGGGACGGCTTGTGGGACACAGGTTGTGAGAGGTGCCTGGGACGGCTTGTGGGGCAC AGGCTGTGAGGGTGCCTGGGACGGCTTGTGGGGCACAGGTTGTGAGAGGTGCCCGGGTCGGCTTGTGGGGCACAGGT TGTGAGAGGTGCCCGGGACGGCTTGTGGGGCACAGGTTGTGAGACGTGCCCGGGACGGCTTGTGGGGCACAGGCTGT GAGGGTGCCCGGGTCGGCTTGTGGGGCACAGGCTGCAAGAGGTGCCCGGGACGGCTTGTGGGGCACAGGCTGTGAGG GTGCCCGGGACGGCTTGTGGGGCACAGGCTGTGAGGGTGCCCGGGACAGCTCGTGGGGCACAGGTTGTGAGAGGTGC CCGGGACGGCTTGTGGGGCACAGGCTGTGAGGGTGCCTGGGACGGCTTGTGGGGCACAGGTTGTGAGAGGTGCCCGG GACGGCTTGTGGGGCACAGGTTGTGAGGATGCCCGGGATGGCTTGTGGGGCACAGGTTGTGAGAGGTGCCTGGGACG GCTTGTGGGGCACAGGCTGTGAGGGTGCCCGGGACGGCTTGTGGGGCACAGGCTGTGAGAGGTGCCTGGGACGGCTT GTGGGGCACAGGCTGTGAGGATGCCCGGGACGGCTTGTGGGGCACAGGTTGTGAGGGGTGCCCAGGACGGCTTGTGG GGCACAGGCTGCAAGAGGTGCCCAGGACGGCTTGTGGGGCACAGGTTGTGAGAGGTGCCCGGGACGGCTTGTGGGGC ACAGGCTGTGAGGGAGCCCGGCACGGCTTGCAGCTACAGGGAGAAAAGACTTGGTGCTGTGGGCCTGCCTTGGGGCT GGTGGTACAGCCCTTATCTGCTGCCCTCAGGATCTCCCGGCCCCTCTCGTCCAGGCCCCTGCAACCCCATGCCCCAG CCTCTGAGGACCAAAGGCGCCCCTGCTTGGGAAGAGGGGGCTCAGGGGAGTCGCCTGACCCGGTTCCAAGCCAGGCT GATTTACCGTTGCTAACATCCTATCGCACGCATCCCTCTGCCTCATGCACCCAACCCCAAGGCCTGGTACACTGCAG GCCCCAAGGTCCTGTGCGTCCTTTCAATACCCTCCTCACCTGCCTCACCTGCCCCCCCTACCCTGACTCTGGCTGGA GACCCCCTCCAGGGAGTTTTCAAAACAAAGGGTGTCAGTCTCCTGTGGGATTCCCTCACCTCTGCAGCCTGCGGTCT GAAAGCTGCCCCATGGTGTGTAGTGCTAAACTTCCAACTTACTCCAGGCCAGCGGTGACAGCCCGAGGGCAGGAAGG GCACCCACACTGAGCCTCAAACAGCTAATTTTGCAACTGTAAGTCCATATAATTGTCTTGAAAAGTAATTTGTTTCA AAAAGCTAAAAAACGAATACTCTTGAGTCTCCTTCTAGTAATTCCCCTTCTAGAGGTCTATCACCAGGAAAAGATCC AAAGCACTGATATTCTTCATGGAGTTGTTTATAATAGAAAAAAACTAGAGCTTGTTCACAAAGGGGAGCTCTGCAGG CTGAAGATGTTGCACCTGTCAGCGGGGATGGGGGCACGCTTGCTGACGCAGCAACGGAAAAGCATCAGTGTGTGAAG ATGCATTTTCTCTCTTTCTATTATTATTATTTTTATTTTTATTTTTTCTGAGGCAGAACCTCGCTCTGTCACCCAGG CTGGAGTGCAGTGATGCGACCTCATCACAACCACGAGCCACCATGTGCGGCCCCATGAGCAAGCCACCACGCCCAGC CTTTTTTTCCCTTGTTTTAAAAAATCCTCTATTTAAAAAAGATGTGCATGGGCCGGGCACGGTGGTTCACGCTCATA ATCCCAGCTCTTTCAGAGGCCGAGGCAGGCAGATCACCTGAGGTCAAGAGTTCGACACCAGCCTGGCCAACATGGTG AAATTCCATCTGTACTAAAAATACAAAAATTAGCCAGGCCGTGGTGGTGTGTGCCTGTAATCCCAGCTACTCAGGAG ACTGAAGCAGGAGAATCACTTGAACCCAGGAGGCAGAGGTTGCAGTGGGTCAAAATCATGCCACCACACTCCAGTCT GGGAGACAGAGCAAGACTCCATCTCAGAAACAAACTAACAAACAAAATTTTTATATCTACCTATAATTCGTATAAAT TTAAAATACATGCATAAAATCATACCCTTTGCAAGCACACGTACTAACTAAAAGGAATATATTCAGCACATAGAAAT GGTTGTCTAACGGAGGAGGGGGGAGTTAATAAACAGAGAGGATAAAAAGAAATAAATCAGTAGAGCTGGAGGAGGGT CTCCTCCAGGCTGCGATGAGAACATAGTGAGCAGAATTGCAGGCCTGCATGACCTCACCTTCTGTGAGGAGTCCGGC CTCCCAAGACGCTTTCCTGCCTAGGTGCCCGGCTCAGAGTGTCCCCTACAAGGCTACTGGAGGAGAACCCCAGACCG AGCCTCATTCAGGTGAGGGGGCTGCACACCGGAGGTGGGAGAGGTCTGTCCCTTCCCACCCTGTGACACTGGGTCCC ACTTTCTCTCTAGGGGGTCTCGGTTTCCTCATTTGCAAACTGGAGCTCATAAGGTGGGCCAGAGAAGTTTCAGTGAA GTGAGGAATGGATCGTCCCTCTGCCAGGGCCCATGTGCTCTAGGTCACCCTGTCATCACAGGGACAGGGAGGTCAAG GACAGTCACTCCTGAGGCCAGTCCGGGCTGGGCTGACCACGTGGACTCTCATGCCCAGATTGGGGCCCCAATCTCCC TGAAGCTGGGGCTCCAGCTGTGACTCAGGGGTGGGCAGAAGGGGAGACAGAAGCGATAGGTTCCTCAGCCCCCAGTC CCACCTGAGGGCCCCTTTGTCACTGGATCTGATAAGAAACACCACCCCTGCAGCCCCCTCCCCTCACCTGACCAATG GCCACAGCCTGGCTGGGCCCAGCTCCCTGTATATAAGGGGACCCTGGGGGCTGAGCACTACCAAGGCCAGTCCTGAG CAGGCCCAACTCCAGTGCAGCCGCCCACCCTGCCGCCATGTCTCTGACCAAGACTTAGGGGACCATCATTGTGTCCA TGTGGGCCAAGATCTCCACGCAGGCCGACACCATCGGCACCGAGACTCTGGAGAGGTGAGTGTCAGATGGGACTGCC AGAGGGACTGGGTGGGAGGCCAGGTATGTGAGTGGGGACAGTGGGGAGCGGGCAGTGGGGAGGGGACCGTGGGGAGG GGACAGTGAGTAGGAGACAGTGGGGAGAGGACAGTGGAGAGGGGACAGTGAGGAGGGGACCATGGGAAGGGGACCGT GGAGTGGGGACAGTGAGGAGGGGACCATAGGGAGGGGACAGTGGGGAGGGGACAGTGAGGAGGGGACCGTGGGGAGG GGACAGTGAGGAGGGGACCGTGGGGAGGAGACAGTGAGGAGGGGACCGTAGGGAGGGGACAGTGAGGAGGGGACCGT GGGGAGGGGACAGTGAGGAGGGGACCGTGGGGAGGGGACAGTGAGGAGGGGACCGTGGGAAGGAGACAGTGAGGAGG GGACCTTGGGGAGGGGACAGTGAGGAGGGGACCATGGGGAGGGGACAGTGAGGAGGGGACAATGGAGAGGGGACAGT GAGGAGGGGACTGTGGGGAGAGGACAGTGAGGAGGGGACCATGGGGAGGGCACAGTGGGGAGGGGAGAGTGAGGAAG GGACAGTGAGGAGGGGACTGTGGGGAGGGGACAGTGGAGACAGATAGCCTTCCCTCTCAGTGAGGAGGGCAGGGTAA GGAGGGAACGATTAGGAGTTGCACAACCATCTGGGCTCGCTGAGACCTGGGCAGGCACAGGCCCAGGTTCTGACAAG CAGAGGGTGAAAGGTTTCGTTCTAGGCCTGAAGGGCCTTACAGGGCAGCCAGGGCACTACAGCCTCTAAAGTCCCAG CATCTGGGATCAGGGCACTGTCCCAGCTTCAAATTCCCAGCATCTGATCCCCTGGGAGGGGCCAGGGAGCTTTTCCT TCCCTGGAACGCTGCTGGGAGGTCATGAGCCTGCAGAAGGGGTGGCGGGCAACCCAGTCTGGGGCTGGGAGGGAGGT CCTGTGGCCAGAGGAGACGGTGGAGGGGCTGGGGGCACCAGGCGTGCTGGAGGCGGAGGGCGGGAGATTTGGGGACC AGGCTGCACAGAACCCGTCGGAAGCAGGGCGATCAGCCGGGAGCTGCAGAGGCCTGGGGGGCCTCTAGCCCAGGGCA GCCTGGGAGGGGCAGCTGCCTGGGCACCCGGGCCCCGCGAGGAGGGGCTGGGGCCTGCTGCGGGGTCGCAGATGTGT CCCGGTGCTCGGAGAGGGCCGCAGGGCGCGTGGGCCGTGGCGGGAGGCCGCGCTGCTGGGAGCTCACGGCCCCCGCC CCCCGTCCCAGGCTCTTCCTCAGCCACCCGCAGACCAAGACCTACTTCCCGCACTTCGACCTGCACCCGGGGTCCGC GCAGTTGCGCGCGCACGGCTCCAAGGTGGTGGCCGCCGTGGGCGACGCGGTGAAGAGCATCGACGACATCGGCGGCG CCCTGTCCAAGCTGAGCGAGCTGCACGCCTACATCCTGCGCGTGGACCCGGTCAACTTCAAGGTGCGCGGGGCGCGG TGCGGGCGGGGCGGGGGGGGGCCGCGGGGGGGGGGGGGCCGCGGGGGGGGGTCGCGGGGGGGGGGGGGTGGGGTCG CGGGGCGGGGGGGGGTCGCGGGGGGGGGCGGGGCGGGGGGGGGGGGCGGGGCGGCCGGGGCCCGGCGGGGCGGGGC GGGGCGGGGAGGGGCTGGGCGGGGCGGGGCGCGGGGGGGGGGGGGCCGGGCCGGGGGGGGTCGCGGGGCGGGGTCG CGGGGCGGGGCGCGGGGCGGGGGGGGGGGGGTGGGGTCGCGGGGGGGGCCCGGGCTAGGCCCCGCCCCCGCACTG AGCCGCCCCCGCCCCCAGCTCCTGTCCCACTGCCTGCTGGTCACCCTGGCCGCGCGCTTCCCCGCCGACTTCACGGC CGAGGCCCACGCCGCCTGGGCCAAGTTCCTATCGGTCGTATCCTCTGTCCTGACCGAGAAGTACCGCTGAGCGCCGC CTCCGGGACCCCCAGGACAGGCTGCGGCCCCTCCCCTGCCCTTCACCCTCCCACAGTTCCTGCCCTGACTCCAATAA ATGGATGAGGACGGAGCGATCTGGGCTCTGTGTTCTCAGTATTGGAGGGAAGGAGGGGAGAAGCTGAGTGATGGGTC CGGGGGCTTCGCAGGAACTCGGTCGTCCCCACTGTCGTCGCGGCCTGGGGTTCACTTGGGGGGCGCCTTGGGGAGGT TCTAGCCCCTGAGCACCGGAGCTGCGGCCCGGGTGGAGCGGAGCAGTCCCGGGCCGGCCCGCGGCGTCTCCTGGGGT CCTTGAGTCGGACGGGCGTTTGTGCGTCTCCCGGCTTCCCATATCGCACAAAGATTGTCACTTCACTAAGCGTATTG GAAGCGTGTCGGGGCTCAGGGAACTTTTCCACAAAGCCTGACGTCCGAATCCCGGGACTCTGGCAGCTACGGGGGTC CCTGAGGCCGGTCCCTCCCCGACTCCTAAGAGAGTAGGGGGTTTCCTGCCCGGTGTTCTCTCTCCGGTTCCTCCCAT GTGCTCCCTCCTGGCAGAGCAGTAACTTTACCCGAGGGGAGTAAACAGATGCCCCTAAAGTCTGCAGTAAAGGTGCC CACGCGCAACGGCGTGGGTCAATGCCAGAAACCCTGGGATCCCGGAGGTCGAGGCCTCCACACAGACGGGAACCCGG GCTGGTTACGTTCCCCGGCGCAGGCCGAGGGTCCCCGCGTTCCCGCCGCGCTCGGGCCGATAAGGACGGGGGGGGTG CCCGGAGGCTCTATAAGGAGGCCAGGGCGGCGGGCGCGGCCCCCAGAGCACGTCAGGCGGCGCCATGCTCAGCGCCC AGGAGCGCGCCCAAATCGCGCAGGTCTGGGACCTGATTGCGGGCCACGAGGCGCAATTCGGGGCGGAGCTGCTGCTC AGGTCGGTAGAGGCGGGGTCTCCGGGAGCTCAGGGAGGTGGAGATGAGGGTTTTGGGCGCGTGGGCCGCCAACGCCA TCCAAGGTCCTTCGGGTGCGGATCCCCGGGCTCTGGGCGGTGTGGGCGCTAGTGAAGCCCCACGCAGCCGCCCTCCT CCCCGGTCACTGACCTGGTCCTGCAGGCTCTTCACGGTGTACCCCAGCACCAAGGTCTACTTCCCGCACCTGAGCGC CTGCCAGGACGCGACGCAGCTGCTGAGCCACGGGCAGCGCATGCTGGCGGCTGTGGGCGCGGCGGTGCAGCACGTGG ACAACCTGCGCGCCGCGCTGAGCCCGCTGGCGGACCTGCACGCGCTCGTGCTGCGCGTGGACCCAGCCAACTTTCCG GTGAGGCCTTTCCGGCCGGGGCAATGGTGCAGCGCGCAGCCGGGGTGGGGGGGCTCTGGGGGTCCCTAGCGGGGCAG ACCCCGTCTCACCGGCCCCTTCTCCTGCAGCTGCTAATCCAGTGTTTCCACGTCGTGCTGGCCTCCCACCTGCAGGA CGAGTTCACCGTGCAAATGCAAGCGGCGTGGGACAAGTTCCTGACTGGTGTGGCCGTGGTGCTGACCGAAAAATACC GCTGAGCCCTGTGCTGCGCAGGCCTTGGTCTGTGCCTGTCAATAAACAGAGGCCCGAACCATCTGCCCCTGCCTGTG TGGTCTTTGGGGAGCTAGCAAAGCGAGGTCACTATTGTTGGCCAGTGAAGCTCAGGGACCTAAAAGGAGCCTCCTAG AACTCTCAAATGCGCCCCACCCCCGGAGGTTTGTCCTCCCATGGCGAGGAGTGCGATGGGGCAGAGGGAGCACTGTG ATGTGGCGGGGGTAGGGAGGGTGGCCTTCGACTTCAACCCTTGAATCGGGCTTCCAACCATACTGTTCGCAAAGCAC TTCCCCATTCACGCATTTATTCATTCATTCTCCCTCCATCCCCACTTCCTGCTGGGACCTGTAGATGCTAATCCTGG CCCTTTTTGCAGAGAGATGCAGAAACTGAGGTCCCAGAGCCAAATGTGCAACCTAATTCGTTGGCCCAGAGCAGAGG GCTCCGCAGACCTGTTCCTTTCCCCTTCCTTCCCCCATGGACACTTCCTCAGTGGCAAACCTGCGCTAGCCTGGTTA GCCCTCCCTGTGACCCTGCAGCCCTGGGGATGAGGTCGGGAGGAAGTCCTCAGTGGCCACAATTTGGCAGACAGAGC AGGTTTAGTCTTCCAGCCTGCTCAATGACAAGCTGTGCGACCCTGGGCGTGTCCCAGAGCTCTCAGGCCTTTACCTA TCGAATAGAAAAACAACGTCCAACTCACGAGATTTTTGAAATAATTTTTGAAATCATAACACAGGGTGGGTGCCTGC AGGGTCGTTGCCACCCCACCCCTCCACCCAGCCCCAGCTGCCGTGTCTCAATCTCTGCAGGTGCCCAGGCCAAGGCA CTCCCTTCCCCAGGTTCCCTCTTCTCCCTCCCCAGGACTGGGAAGGGAATCTTAGGGCTCCACCCCAGGCTTTTCAG ACAAAGAATAGGGGCTGAGGAAAGAGTGGGACCTTGGAGGTCTCCAAACCCTGAATAGGGTTGGCTCTGGGTTGGCC ATCCTGGGTCTGTGTGGGGAGCACTGGACCAGGCCTGGCACCCAGGTCTGACCTGGCAGTCAGCAACGAGGTCTGAA GAGAGCTGCTGGAAGTGGAGCCCTGACTGTGAGTCGGCCAAACTCCCCCCAGCAGTCAGTGCCAGTGACCTGTTGCC CTGCACTGCCTGGGACCCCAGCCCGGTAGTTTGGAGAACTTGGCCCCACGTTATCTACATCCCCCAACTGTTTTTTT GTTTTTGGGGGTTTTTTTTTTTTTTGCTTTGTTTTTGTTTTTGAGATAGGCCCTTGCTCTGACACCCCGGCTGGAGT GCAGTGGCACAGTTTTGGCTCACTGCAGCCTCAACCTCCTGGGTTCAAGCGATTCTCCTGCCTCTGTCTCCCGTGTA GCTGGGATTACAGGCATGGGCCGCCATTCCTGGCTAATTTTTGTATTTTTAATAGAGACACAGTTTCACCATGTTGA TCAGGCTGGTCTCAAACTCCTGACCTCAAGTGATCTGCCCTCCTCGGTCTCCCAAAGTGCTGGGATGACAGGCGTGA GCCACCACACCCAGCCCCCGCAACTGTTTACATGGATAATTAACAGCTTTTTGTCCCAGGCAGAGTTTGGTGTGAAA GCAGCTTATGTTTCACTTTGGAAAAACTGTGCTCTTCTCCCCATCCAGGAAGCTGCCTGGGTCTGGGCCATATGTGG ATACCTTATGGGTATAAGCTGCTCAGGACCCTGTGTGGAAGCTCAGGACAATGCCAGCGGGAAGGCTACCATGTGGA GAGCTGGTCTCTGTTTGGGCAGGACTAAGAGACGCAGGGCAGCCTTGGGCAACCTGTCTACTCTCACTCACTCCTCC TCCCCTTTCCTGTGCCAGGCACCTCCTGGCAACTTGCCAGCCAATGACCCTGCATCCCAGGCATAAGAGCTCCTACT CTCCCCCACCTTTCACTTTTGAGCTTACACAGACTCAGAAATAAGCTGCCGTGGTGCTGTCTCCTGAGGACAAGGCT AACACCAAGGCGGTCTGGGAGAAAGTTGGCAACCACACTGCTGGCTATGCCACGGAGGCCCTGGAGAGGCAAGAACC CTCCTCTCCCTGCTCACACCTTGGGTCCAACGCCCACTCCAGGGCTCCACTGGCCACCCCTAACTATTCTTACCCTG GACCCAGCCCCCAGCCCCTCACTCTTTGCTTCCCCCTGAAGCATGTTCCTGACCTTCCTCTCACTTGGCCCTGAGTT ATGGCTCAGCCCAGATCAAGAAACAATGCAAGTAGGTGGCCGACACGCTGACCAATGCCGTGGTCCACTTAGATGAC ATGCCCAATGATGTGTCTGAGCTGAGGAAGCTGCATGTCCACGAGCTGTGGGTGGACCCAGGCAACATCAGGGAGAG CTTTGGGCTGGGAGGAATCTAGGGTGTGGGGGCAGCTGGCCTTCCTCATAGGACAGACCCTCCCACGCGTTCAGGGA GGTGGAGCACAGGTGGCAGTAGTATCTGCATCCCCTGACTCTCTCTCCACAGTTCCTGGGTAAATGCCTGCTGGTGA CCTAGGCCTGCCACACCCTTCCCAGTTTACCCATGTGGTGCCTCCATGGACAAATTATTTGCTTTTGTGAGTGCTGT GTTGACCTAAAAACACCATTAAGCTAGAGCATTGGTGGTCATGCCCCCTGCCTGCTGGGCCTCCCACCAGGCCCTCC TCCCCTCCCTGCCCCAGCACTTCCTGATCTTTGAATGAAGTCCGAGTAGGCAGCAGCCTGTGTGTGCCTGGGTTCTC TCTGTCCCGGAATGTGCCAACAGTGGAGGTGTTTACCTGTCTCAGACCAAGGACCTCTCTGCAGCTGCATGGGGCTG GGGAGGGAGAACTGCAGGGAGTATGGGAGGGGAAGCTGAGGTGGGCCTGCTCAAGAGAAGGTGCTGAACCATCCCCT GTCCTGAGAGGTGCCAGGCCTGCAGGCAGTGGCTCAGAAGCTGGGGAGGAGAGAGGCATCCAGGGTTCTACTCAGGG AGTCCCAGCATCGCCACCCTCCTTTGAAATCTCCCTGGTTGAACCCAGTTAACATACGCTCTCCATCAAAACAAAAC GAAACAAAACAAACTAGCAAAATAGGCTGTCCCCAATGCAAGTGCAGGTGCCAGAACATTTCTCTCATTCTCACCCC TTCCTGCCAGAGGGTAGGTGGCTGGAGTGAGGGTGCTGGCCCTACTCACACTTCCTGTGTCATGGTGACCCTCTGAG AGCAGCCCAGTCAGTGGGGAAGGAGGAAGGGGCTGGGATGCTCACAGCCGGCAGCCCACACCTGGGGAGACTCTTCA GCAGAGCACCTTGCGGCCTTACTCCTGCACGTCTCCTGCAGTTTGTAAGGTGCATTCAGAACTCACTGTGTGCCCAG CCCTGAGCTCCCAGCTAATTGCCCCACCCAGGGCCTCTGGGACCTCCTGGTGCTTCTGCTTCCTGTGCTGCCAGCAA CTTCTGGAAACGTCCCTGTCCCCGGTGCTGAAGTCCTGGAATCCATGCTGGGAAGTTGCACAGCCCATCTGGCTCTC AGCCAGCCTAGGAACACGAGCAGCACTTCCAGCCCAGCCCCTGCCCCACAGCAAGCCTCCCCCTCCACACTCACAGT ACTGAATTGAGCTTTGGGTAGGGTGGAGAGGACCCTGTCACCGCTTTTCTTCTGGACATGGACCTCTCTGAATTGTT GGGGAGTTCCCTCCCCCTCTCCACCACCCACTCTTCCTGTGCCTCACAGCCCAGAGCATTGTTATTTCAACAGAAAC ACTTTAAAAAATAAACTAAAATCCGACAGGCACGGTGGCTCACACCTGTAATCCCAGTACTTTGGGAGGCTGAGGCG AGAGGATCACCTGAGGTCGGGAGTTTGAGACCAGCCTGACCAATATGGAGAAACCCCAGTTATACTAAAAATACAAA ATTAGCTGGGTGTGGTGGCGCATGCCTGTAATCCTAGCTACTAGGAAGGCTGAGGCAGGAGAATCGCTTGAACCCGG GAGGTGGAGGTTGAGGTGAGCTGAGATCACGCCATTGCACTCCAGCCTGGGCAACAAGAGCAAAACTCCGTCTCAAA AAATAAATAAATAAATAAATAAATAAACTAAAATCTATCCATGCTTTCACACACACACACACACACACACACACACA CCCTTTTTTGTGTTACTTAAAGTAGGAGAGTGTCTCTCTTTCCTGTCTCCTCACACCCACCCCCAGAAGAGACCAAA ATGAAGGGTTTGGAACTCAGCCCATGGGCCCCATCCCATGCTGAGGGAACACAGCTACATCTACAACTACTGCCACA GGCTCTCTTTTTGGACAAAAATACCATCATACTGTAGATACCTGTGTACAACTTCCTATTCTCAGTGAAGTGTCTCC CCTGCATCCCTTTCAGCCAGTTCATTCAGCTCTGCGCCATTCCACAGTCTCACTGATTATTACTATGTTTCCATCAT GATCCCCCCAAAAAATCATGACTTTATTTTTTTATTTTTATTATTATTATTTTTTTTTTTTTTTTTGTGACGGAGTC TCGCTCTGTCACCCAGGCTGGAGTGCAGTGGCACAATCTCGGCTCACTGCAAGCTCCACCTCGCAGGTTCACGCCAT TCTCCTCCCTCAGCCTCCCGAGTAGCTGAGTAGCTGGGACTACAGGCGCCCCCCACTACGCCTGGCTAATTTTTTCT ATTTTTAATAGAGACAGAGTTTCACTGCATTAGCGAGGATGGTCTCGATCTCCTGACCTCGCATCTGCCCGCCTCAG CCTCCCAATGTGCTGGGATTACAGGCGTGAGCCACCGCGCCCGGCCTTATGTATTTATTTTTTTGAGACAGAGTCTC GCTGTGTCGTCAGGCTAGAGTGCTGTGGCACGATCTCGGCTCACTGCAACCTCCAACTCCCTGGTTCAAAGGATTCT CCAGCCTCCACCTCCCGAGTAGCTGGGATTACAGGCGTGCACCACCACACCCAGCTAATTTTTGTATTTTTAGTAGA GACGGGGTTTCTCCATGTTGGTCAGCCTGGTCTCGAACTCCCGACCTCAGCTGATCCACCCGCCTTGGCCTCCCAAA GTGCTGGGATTACAGGCGTGAGCCACCGAGCCTGGCCAAACCATCACTTTTCATGAGCAGGGATGCACCCACTGGCA CTCCTGCACCTCCCACCCTCCCCCTCGCCAAGTCCACCCCTTCCTTCCTCACCCCACATCCCCTCACCTACATTCTG CAACCACAGGGGCCTTCTCTCCCCTGTCCTTTCCCTACCCAGAGCCAAGTTTGTTTATCTGTTTACAACCAGTATTT ACCTAGCAAGTCTTCCATCAGATAGCATTTGGAGAGCTGGGGGTGTCACAGTGAACCACGACCTCTAGGCCAGTGGG AGAGTCAGTCACACAAACTGTGAGTCCATGACTTGGGGCTTAGCCAGCACCCACCACCCCACGCGCCACCCCACAAC CCCGGGTAGAGGAGTCTGAATCTGGAGCCGCCCCCAGCCCAGCCCCGTGCTTTTTGCGTCCTGGTGTTTGTTCCTTC CCGGTGCCTGTCACTCAAGCACACTAGTGACTATCGCCAGAGGGAAAGGGAGCTGCAGGAAGCGAGGCTGGAGAGCA GGAGGGGCTCTGCGCAGAAATTCTTTTGAGTTCCTATGGGCCAGGGCGTCCGGGTGCGCGCATTCCTCTCCGCCCCA GGATTGGGCGAAGCCCTCCGGCTCGCACTCGCTCGCCCGTGTGTTCCCCGATCCCGCTGGAGTCGATGCGCGTCCAG CGCGTGCCAGGCCGGGGCGGGGGTGCGGGCTGACTTTCTCCCTCGCTAGGGACGCTCCGGCGCCCGAAAGGAAAGGG TGGCGCTGCGCTCCGGGGTGCACGAGCCGACAGCGCCCGACCCCAACGGGCCGGCCCCGCCAGCGCCGCTACCGCCC TGCCCCCGGGCGAGCGGGATGGGCGGGAGTGGAGTGGCGGGTGGAGGGTGGAGACGTCCTGGCCCCCGCCCCGCGTG CACCCCCAGGGGAGGCCGAGCCCGCCGCCCGGCCCCGCGCAGGCCCCGCCCGGGACTCCCCTGCGGTCCAGGCCGCG CCCCGGGCTCCGCGCCAGCCAATGAGCGCCGCCCGGCCGGGCGTGCCCCCGCGCCCCAAGCATAAACCCTGGCGCGC TCGCGGGCCGGCACTCTTCTGGTCCCCACAGACTCAGAGAGAACCCACCATGGTGCTGTCTCCTGCCGACAAGACCA ACGTCAAGGCCGCCTGGGGTAAGGTCGGCGCGCACGCTGGCGAGTATGGTGCGGAGGCCCTGGAGAGGTGAGGCTCC CTCCCCTGCTCCGACCCGGGCTCCTCGCCCGCCCGGACCCACAGGCCACCCTCAACCGTCCTGGCCCCGGACCCAAA CCCCACCCCTCACTCTGCTTCTCCCCGCAGGATGTTCCTGTCCTTCCCCACCACCAAGACCTACTTCCCGCACTTCG ACCTGAGCCACGGCTCTGCCCAGGTTAAGGGCCACGGCAAGAAGGTGGCCGACGCGCTGACCAACGCCGTGGCGCAC GTGGACGACATGCCCAACGCGCTGTCCGCCCTGAGCGACCTGCACGCGCACAAGCTTCGGGTGGACCCGGTCAACTT CAAGGTGAGCGGCGGGCCGGGAGCGATCTGGGTCGAGGGGCGAGATGGCGCCTTCCTCTCAGGGCAGAGGATCACGC GGGTTGCGGGAGGTGTAGCGCAGGCGGCGGCTGCGGGCCTGGGCCGCACTGACCCTCTTCTCTGCACAGCTCCTAAG CCACTGCCTGCTGGTGACCCTGGCCGCCCACCTCCCCGCCGAGTTCACCCCTGCGGTGCACGCCTCCCTGGACAAGT TCCTGGCTTCTGTGAGCACCGTGCTGACCTCCAAATACCGTTAAGCTGGAGCCTCGGTAGCCGTTCCTCCTGCCCGC TGGGCCTCCCAACGGGCCCTCCTCCCCTCCTTGCACCGGCCCTTCCTGGTCTTTGAATAAAGTCTGAGTGGGCAGCA GCCTGTGTGTGCCTGGGTTCTCTCTATCCCGGAATGTGCCAACAATGGAGGTGTTTACCTGTCTCAGACCAAGGACC TCTCTGCAGCTGCATGGGGCTGGGGAGGGAGAACTGCAGGGAGTATGGGAGGGGAAGCTGAGGTGGGCCTGCTCAAG AGAAGGTGCTGAACCATCCCCTGTCCTGAGAGGTGCCAGGCCTGCAGGCAGTGGCTCAGAAGCTGGGGAGGAGAGAG GCATCCAGGGTTCTACTCAGGGAGTCCCAGCATCGCCACCCTCCTTTGAAATCTCCCTGGTTGAACCCAGTTAACAT ACGCTCTCCATCAAAACAAAACGAAACAAAACAAACTAGCAAAATAGGCTGTCCCCAGTGCAAGTGCAGGTGCCAGA ACATTTCTCTCATTCCCACCCCTTCCTGCCAGAGGGTAGGTGGCTGGAGTGAGGGTGCTGGCCCTACTCACACTTCC TGTGTCACGGTGACCCTCTGAGAGCAGCCCAGTCAGTGGGGAAGGAGGAAGGGGCTGGGATGCTCACAGCCGGCAGC CCACACCTGGGGAGACTCTTCAGCAGAGCACCTTGCGGCCTTACTCCTGCACGTCTCCTGCAGTTTGTAAGGTGCAT TCAGAACTCACTGTGTGCCCAGCCCTGAGCTCCCAGCTAATTGCCCCACCCAGGGCCTCTGGGACCTCCTGGTCTTC TGCTTCCTGTGCTGCCAGCAACTTCTGGAAACGTCCCTGTCCCCGGTGCTGAAGTCCTGGAATCCATGCTGGGAAGT TGCACAGCCCATCTGGCTCTCAGCCAGCCTAGGAACATGAGCAGCACTTCCAACCCAGTCCCTGCCCCACAGCAAGC CTCCCCCTCCACACTCACAGTACTGGATTGAGCTTTGGGGAGGGTGGAGAGGACCCTGTCACTGCTTTCCTTCTGGA CATGGACCTCTCTGAATTGTTGGGGAGTTCCCTCCCCTCTCCACCACCCGCTCTTCCTGCGCCTCACAGCCCAGAGC ATTGTTATTTCAGCAGAAACACTTTAAAAAATAAACTAAAATCCGACAGGCACGGTGGCTCACGCCTGTAATCCCAG CACTTTGGGAGGCCGAGGTGGGAGGATCACCTGAGGTCGGGAGTTTGAGACCACCCTGATCAACATGTAGAAACCCC ATCTATACTAAAAATACAAAATCAGCCGGGCATGGTGGCCCATGCCTGTAAACCCACCTACTCCGGAGGCTGAGGCA GGAGAATCATTTTAACCAAGGAGGCAGAGGTTGCAGTGAGCTAAGATCACACCATTGCACTCCAGCCTGGAAAACAA CAGCGAAACTCCGCCTCAAAAAAAAAAAAGCCCCCACATCTTATCTTTTTTTTTTCCTTCAGGCTGTGGGCAGAGTC AGAAGAGGGTGGCAGACAGGGAGGGGAAATGAGAAGATCCAACGGGGGAAGCATTGCTAAGCTGGTCGGAGCTACTT CCTTCTCTGCCCAAGGCAGCTTACCCTGGCTTGCTCCTGGACACCCAGGGCAGGGCCTGAGTAAGGGCCTGGGGAGA CAGGGCAGGGAGCAGGCTGAAGGGTGCTGACCTGATGCACTCCTCAAAGCAAGATCTTCTGCCAGACCCCCAGGAAA TGACTTATCAGTGATTTCTCAGGCTGTTTTCTCCTCAGTACCATCCCCCCAAAAAACATCACTTTTCATGCACAGGG ATGCACCCACTGGCACTCCTGCACCTCCCACCCTTCCCCAGAAGTCCACCCCTTCCTTCCTCACCCTGCAGGAGCTG GCCAGCCTCATCACCCCAACATCTCCCCACCTCCATTCTCCAACCACAGGGCCCTTGTCTCCTCTGTCCTTTCCCCT CCCCGAGCCAAGCCTCCTCCCTCCTCCACCTCCTCCACCTAATACATATCCTTAAGTCTCACCTCCTCCAGGAAGCC CTCAGACTAACCCTGGTCACCTTGAATGCCTCGTCCACACCTCCAGACTTCCTCAGGGCCTGTGATGAGGTCTGCAC CTCTGTGTGTACTTGTGTGATGGTTAGAGGACTGCCTACCTCCCAGAGGAGGTTGAATGCTCCAGCCGGTTCCAGCT ATTGCTTTGTTTACCTGTTTAACCAGTATTTACCTAGCAAGTCTTCCATCAGATAGCATTTGGAGAGCTGGGGGTGT CACAGTGAACCACGACCTCTAGGCCAGTGGGAGAGTCAGTCACACAAACTGTGAGTCCATGACTTGGGGCTTAGCCA GCACCCACCACCCCACGCGCCACCCCACAACCCCGGGTAGAGGAGTCTGAATCTGGAGCCGCCCCCAGCCCAGCCCC GTGCTTTTTGCGTCCTGGTGTTTATTCCTTCCCGGTGCCTGTCACTCAAGCACACTAGTGACTATCGCCAGAGGGAA AGGGAGCTGCAGGAAGCGAGGCTGGAGAGCAGGAGGGGCTCTGCGCAGAAATTCTTTTGAGTTCCTATGGGCCAGGG CGTCCGGGTGCGCGCATTCCTCTCCGCCCCAGGATTGGGCGAAGCCTCCCGGCTCGCACTCGCTCGCCCGTGTGTTC CCCGATCCCGCTGGAGTCGATGCGCGTCCAGCGCGTGCCAGGCCGGGGCGGGGGTGCGGGCTGACTTTCTCCCTCGC TAGGGACGCTCCGGCGCCCGAAAGGAAAGGGTGGCGCTGCGCTCCGGGGTGCACGAGCCGACAGCGCCCGACCCCAA CGGGCCGGCCCCGCCAGCGCCGCTACCGCCCTGCCCCCGGGCGAGCGGGATGGGGGGAGTGGAGTGGCGGGTGGAG GGTGGAGACGTCCTGGCCCCCGCCCCGCGTGCACCCCCAGGGGAGGCCGAGCCCGCCGCCCGGCCCCGCGCAGGCCC CGCCCGGGACTCCCCTGCGGTCCAGGCCGCGCCCCGGGCTCCGCGCCAGCCAATGAGCGCCGCCCGGCCGGGCGTGC CCCCGCGCCCCAAGCATAAACCCTGGCGCGCTCGCGGCCCGGCACTCTTCTGGTCCCCACAGACTCAGAGAGAACCC ACCATGGTGCTGTCTCCTGCCGACAAGACCAACGTCAAGGCCGCCTGGGGTAAGGTCGGCGCGCACGCTGGCGAGTA TGGTGCGGAGGCCCTGGAGAGGTGAGGCTCCCTCCCCTGCTCCGACCCGGGCTCCTCGCCCGCCCGGACCCACAGGC CACCCTCAACCGTCCTGGCCCCGGACCCAAACCCCACCCCTCACTCTGCTTCTCCCCGCAGGATGTTCCTGTCCTTC CCCACCACCAAGACCTACTTCCCGCACTTCGACCTGAGCCACGGCTCTGCCCAGGTTAAGGGCCACGGCAAGAAGGT GGCCGACGCGCTGACCAACGCCGTGGCGCACGTGGACGACATGCCCAACGCGCTGTCCGCCCTGAGCGACCTGCACG CGCACAAGCTTCGGGTGGACCCGGTCAACTTCAAGGTGAGCGGCGGGCCGGGAGCGATCTGGGTCGAGGGGCGAGAT GGCGCCTTCCTCGCAGGGCAGAGGATCACGCGGGTTGCGGGAGGTGTAGCGCAGGCGGCGGCTGCGGGCCTGGGCCC TCGGCCCCACTGACCCTCTTCTCTGCACAGCTCCTAAGCCACTGCCTGCTGGTGACCCTGGCCGCCCACCTCCCCGC CGAGTTCACCCCTGCGGTGCACGCCTCCCTGGACAAGTTCCTGGCTTCTGTGAGCACCGTGCTGACCTCCAAATACC GTTAAGCTGGAGCCTCGGTGGCCATGCTTCTTGCCCCTTGGGCCTCCCCCCAGCCCCTCCTCCCCTTCCTGCACCCG TACCCCCGTGGTCTTTGAATAAAGTCTGAGTGGGCGGCAGCCTGTGTGTGCCTGAGTTTTTTCCCTCAGCAAACGTG CCAGGCATGGGCGTGGACAGCAGCTGGGACACACATGGCTAGAACCTCTCTGCAGCTGGATAGGGTAGGAAAAGGCA GGGGCGGGAGGAGGGGATGGAGGAGGGAAAGTGGAGCCACCGCGAAGTCCAGCTGGAAAAACGCTGGACCCTAGAGT GCTTTGAGGATGCATTTGCTCTTTCCCGAGTTTTATTCCCAGACTTTTCAGATTCAATGCAGGTTTGCTGAAATAAT GAATTTATCCATCTTTACGTTTCTGGGCACTCTTGTGCCAAGAACTGGCTGGCTTTCTGCCTGGGACGTCACTGGTT TCCCAGAGGTCCTCCCACATATGGGTGGTGGGTAGGTCAGAGAAGTCCCACTCCAGCATGGCTGCATTGATCCCCCA TCGTTCCCACTAGTCTCCGTAAAACCTCCCAGATACAGGCACAGTCTAGATGAAATCAGGGGTGCGGGGTGCAACTG CAGGCCCCAGGCAATTCAATAGGGGCTCTACTTTCACCCCCAGGTCACCCCAGAATGCTCACACACCAGACACTGAC GCCCTGGGGCTGTCAAGATCAGGCGTTTGTCTCTGGGCCCAGCTCAGGGCCCAGCTCAGCACCCACTCAGCTCCCCT GAGGCTGGGGAGCCTGTCCCATTGCGACTGGAGAGGAGAGCGGGGCCACAGAGGCCTGGCTAGAAGGTCCCTTCTCC CTGGTGTGTGTTTTCTCTCTGCTGAGCAGGCTTGCAGTGCCTGGGGTATCAGAGGGAGGGTTCCCGGAGCTGGTAGC CATAAAGCCCTGGCCCTCAACTGATAGGAATATCTTTTATTCCCTGAGCCCATGAATCACCCTTGGTAAACACCTAT GGCAGGCCCTCTGCCTGCGTTTGTGATGTCCTTCCCGCAGCCTGTGGGTACAGTATCAACTGTCAGGAAGACGGTGT CTTCGTTATTTCATCAGGAAGAATGGAGGTCTGACCTAAAGGTAGAAATATGTCAAATGTACAGCAGAGGGCTGGTT GGAGTGCAGCGCTTTTTACAATTAATTGATCAGAACCAGTTATAAATTTATCATTTCCTTCTCCACTCCTGCTGCTT CAGTTGACTAAGCCTAAGAAAAAATTATAAAAATTGGCCGGGCGCGGTGGCTCACACCTGTAATTGCAGCACTTTGC CAGGCTTAGGCAGGTGGATCACCTGAAGTCAGGGGTTCGAGACCAGCCTAGCCAACATAGTGAAACCCTGTCTCTAC TAAAAAGACAAAAATTGTCCAGGTGTGATGACTCATGCCTGTAAACCTGGCACTTTGGGAGGCGGAGGTTGTAGTGA GTCAAGATCGCGCCATCGCACTCCAGCTTGGGCAACAAGAGCGAAACTCTGTCTCAAAAAAAAATTTAATCTAATTT AATTTAATTTAAAAATTAGCACGGTGGTTGGGCACAGTGGCTCACGCCTGTAATCCCAGCACTTTGGGAAGCCAAGG TGGGCAGATCACAAGGTCAGGAATTCGAGACCAGCCTGGCCAATATGGGGAAACCCCATCTCTACTAAAAATACAAA AAATTAGCCGGGTGTGGTGGCGCACGCCTGTAATCCCAGCTACTCGGGAGGTTGAGGTAGGAGAATCACTTGAACCC AGGAGGCAGAGGTTGCAGTGACCCGAGATCACACCATTGCACTCTAGCCTGGGCAACAAGAGCAAAACTCCATCTCA AAAAAAATTATAAAAATTATACATCAGTAGATGAATGGGTAAACAAAATGTGGTGGTCTATACACACAATGGAATAT TATTTGGCCACAAAAAGAAATGAAGCACTGATAGGATGTAGCTGCACCCTGAAAATATTTGACAAGTAAAAGAAGCC GGACACCAAAGGTCACAAACTGCATGACCCCATCTATATGCAATATCCGCTACAGCCAAATCCATAGGGACCAAAAG CGGATTAGTGGCTGCCGGGGCCAGAGTTACTGTTAATGAGTACCGAGGTGGCGTTTGGGATGATGAAAAAGTTCTGA CCTAGATAGTGGTGATGGCTGCATAACACTAAGTGTTCTTAATATCACCAAATTTTATACCTGAAAAATGGCTACAA TGGTAATTTATGTCTATTTTATCACCTTTTTTAAAACAAAAAAGATATAAGGGGTACAGCAGAGTGAGTGCTGCATA TGCATTTACTATTATTCTTGGGTTACATCCCAGGTACTCAATAAATGTTCACTGCCCTGAAGAAACACCTGCTACGA GTCAGGCACCTCACAGTTGTTATCCGTTTAATTCTCACAATCTGAGAAGAAACTGTCACCCTCATTTTATATAATAA ATGAGAAAACAGACTCGGGCAAGTGTCACAATAGAATCAAGAGGCAGAATAAACTGACTTCCAATGCCAAATCCATG CCGAAATTCAGTGCTATAATAATGTACATGGCCGGGCGCGGTGGTTCACGCCTGTAATCCCAGAACTTTGGGAGGCT GAGGCGGGAGGATCACCTGAGGTCGGGAGTTTGAGATCAGCCTAACACGGTGAAACCCTGTCTCTACTAAAAATACA AAATTGGCATGGTGGCATGCACCTGTGATCCCAGTTACTCGGGAGGCTGAGGCAGGAGAATCGTTTGAACCCGGGAG GCGGAGGTTGCAGTGAGCCGGAATGGCGCCACTGCACTCACCGCACCCGGCCAATTTTTGTGTTTTTAGTAGAGACT AAATACCATATAGTGAACACCTAAGACGGGGGGCCTTGGATCCAGGGCGATTCAGAGGGCCCCGGTCGGAGCTGTCG GAGATTGAGCGCGCGCGGTCCCGGGATCTCCGACGAGGCCCTGGACCCCCGGGCGGCGAAGCTGCGGCGCGGCGCCC CCTGGAGGCCGCGGGACCCCTGGCCGGTCCGCGCAGGCGCAGCGGGGTCGCAGGGCGCGGCGGGTTCCAGCGCGGGG ATGGCGCTGTCCGCGGAGGACCGGGCGCTGGTGCGCGCCCTGTGGAAGAAGCTGGGCAGCAACGTCGGCGTCTACAC GACAGAGGCCCTGGAAAGGTGCGGCAGGCTGGGCGCCCCCGCCCCCAGGGGCCCTCCCTCCCCAAGCCCCCCGGACG CGCCTCACCCACGTTCCTCTCGCAGGACCTTCCTGGCTTTCCCCGCCACGAAGACCTACTTCTCCCACCTGGACCTG AGCCCCGGCTCCTCACAAGTCAGAGCCCACGGCCAGAAGGTGGCGGACGCGCTGAGCCTCGCCGTGGAGCGCCTGGA CGACCTACCCCACGCGCTGTCCGCGCTGAGCCACCTGCACGCGTGCCAGCTGCGAGTGGACCCGGCCAGCTTCCAGG TGAGCGGCTGCCGTGCTGGGCCCCTGTCCCCGGGAGGGCCCCGGCGGGGTGGGTGCGGGGGGCGTGCGGGGGGGGTG CAGGCGAGTGAGCCTTGAGCGCTCGCCGCAGCTCCTGGGCCACTGCCTGCTGGTAACCCTCGCCCGGCACTACCCCG GAGACTTCAGCCCCGCGCTGCAGGCGTCGCTGGACAAGTTCCTGAGCCACGTTATCTCGGCGCTGGTTTCCGAGTAC CGCTGAACTGTGGGTGGGTGGCCGCGGGATCCCCAGGCGACCTTCCCCGTGTTTGAGTAAAGCCTCTCCCAGGAGCA GCCTTCTTGCCGTGCTCTCTCGAGGTCAGGACGCGAGAGGAAGGCGCCGCCCCTCCCCAAGGAAAGGCGAGGGCCTG GGGCACACCCCCAGTGCCCAGATCCAGGCGCGCCTCTTTCCACCTCCAGCAGGTTTGGGGCCTCGGCCATGGGGGCA CCGAACTGCGTGCAGCCTGACCCTCCCGAATGGGGTGGTAGGTGAGGGCCGCGGGACGCCCCGGGCGGCGGGCTGCG AGGACGGCCGACTCTGCCCATCCCGAGGGCGGCTGGCTTCGCCCTCCCCACTCTGCGCCGAGCACGCGGCCCGGACC CACCGCGAGAACTCCGCACCTGCAGCGTGAACGCACGCGGGCGGCGTTAAGGGCCCGGGGCTGACTCGGAGCAGGTT AGGGAACAGCGCCCCCTCCCGGCGCGAGCCGGTACCTGCGCAGCACCCAGCCGCCGCGGCTGTGGCCTGGAATCGGG GACCTGGGGTGCCGGGGGGTTGTGGTGAAGGAGGTGGGACCAGCCCCAGCACCTAGCCACGTAGCTGGCGAGGTGGA CCAGGAACCGACCCAGACCCCTGCCGTCACCCGACATCACTACGGAGAGTGAAGCTTTTTTATATTTGTCCACATAA AACCAATCATGGTCATTGTAGAACTTCCGAAAACAAGGCTTGCTGCACCTTCCTGTGTATCCCAGGTCCAGGAATGG GTGCAGCACATCCTTCAGCTGCCGCTTGACACGCGGCAAACTGTGTCATGTGTAAACAAGAACAGGACATGGCTGTC ATATCCAAGAGCACATGTGTAACACAGACATGCCACACACACACACACACACACACGGGGTAGAGGCAGGCCTCATC CACACCCCTAACATTTGATGCGTAGCTGTTCCAGTCTTCTAGGCACATGTAGAGATGCTTTTCCTCAGAAATGGTAT TCTCAAGGTGACACTGAGGAAAAGTGGACAGGCCGGGCGCGGTGGCTCACGCCTGTAATCCCAGCACTCCGGGAGGC CGAGGCGGGCGGATC

GenBank Accession No. NM_000517 ACTCTTCTGGTCCCCACAGACTCAGAGAGAACCCACCATGGTGCTGTCTCCTGCCGACAAGACCAACGTCAAGGCCG CCTGGGGTAAGGTCGGCGCGCACGCTGGCGAGTATGGTGCGGAGGCCCTGGAGAGGATGTTCCTGTCCTTCCCCACC ACCAAGACCTACTTCCCGCACTTCGACCTGAGCCACGGCTCTGCCCAGGTTAAGGGCCACGGCAAGAAGGTGGCCGA CGCGCTGACCAACGCCGTGGCGCACGTGGACGACATGCCCAACGCGCTGTCCGCCCTGAGCGACCTGCACGCGCACA AGCTTCGGGTGGACCCGGTCAACTTCAAGCTCCTAAGCCACTGCCTGCTGGTGACCCTGGCCGCCCACCTCCCCGCC GAGTTCACCCCTGCGGTGCACGCCTCCCTGGACAAGTTCCTGGCTTCTGTGAGCACCGTGCTGACCTCCAAATACCG TTAAGCTGGAGCCTCGGTAGCCGTTCCTCCTGCCCGCTGGGCCTCCCAACGGGCCCTCCTCCCCTCCTTGCACCGGC CCTTCCTGGTCTTTGAATAAAGTCTGAGTGGGCAGCA

GenBank Accession No. J00179 GAATTCTAATCTCCCTCTCAACCCTACAGTCACCCATTTGGTATATTAAAGATGTGTTGTCTACTGTCTAGTATCCC TCAAGTAGTGTCAGGAATTAGTCATTTAAATAGTCTGCAAGCCAGGAGTGGTGGCTCATGTCTGTAATTCCAGCACT GGAGAGGTAGAAGTGGGAGGACTGCTTGAGCTCAAGAGTTTGATATTATCCTGGACAACATAGCAAGACCTCGTCTC TACTTAAAAAAAAAAAAATTAGCCAGGCATGTGATGTACACCTGTAGTCCCAGCTACTCAGGAGGCCGAAATGGGAG GATCCCTTGAGCTCAGGAGGTCAAGGCTGCAGTGAGACATGATCTTGCCACTGCACTCCAGCCTGGACAGCAGAGTG AAACCTTGCCTCACGAAACAGAATACAAAAACAAACAAACAAAAAACTGCTCCGCAATGCGCTTCCTTGATGCTCTA CCACATAGGTCTGGGTACTTTGTACACATTATCTCATTGCTGTTCGTAATTGTTAGATTAATTTTGTAATATTGATA TTATTCCTAGAAAGCTGAGGCCTCAAGATGATAACTTTTATTTTCTGGACTTGTAATAGCTTTCTCTTGTATTCACC ATGTTGTAACTTTCTTAGAGTAGTAACAATATAAAGTTATTGTGAGTTTTTGCAAACACAGCAAACACAACGACCCA TATAGACATTGATGTGAAATTGTCTATTGTCAATTTATGGGAAAACAAGTATGTACTTTTTCTACTAAGCCATTGAA ACAGGAATAACAGAACAAGATTGAAAGAATACATTTTCCGAAATTACTTGAGTATTATACAAAGACAAGCACGTGGA CCTGGGAGGAGGGTTATTGTCCATGACTGGTGTGTGGAGACAAATGCAGGTTTATAATAGATGGGATGGCATCTAGC GCAATGACTTTGCCATCACTTTTAGAGAGCTCTTGGGGACCCCAGTACACAAGAGGGGACGCAGGGTATATGTAGAC ATCTCATTCTTTTTCTTAGTGTGAGAATAAGAATAGCCATGACCTGAGTTTATAGACAATGAGCCCTTTTCTCTCTC CCACTCAGCAGCTATGAGATGGCTTGCCCTGCCTCTCTACTAGGCTGACTCACTCCAAGGCCCAGCAATGGGCAGGG CTCTGTCAGGGCTTTGATAGCACTATCTGCAGAGCCAGGGCCGAGAAGGGGTGGACTCCAGAGACTCTCCCTCCCAT TCCCGAGCAGGGTTTGCTTATTTATGCATTTAAATGATATATTTATTTTAAAAGAAATAACAGGAGACTGCCCAGCC CTGGCTGTGACATGGAAACTATGTAGAATATTTTGGGTTCCATTTTTTTTTCCTTCTTTCAGTTAGAGGAAAAGGGG CTCACTGCACATACACTAGACAGAAAGTCAGGAGCTTTGAATCCAAGCCTGATCATTTCCATGTCATACTGAGAAAG TCCCCACCCTTCTCTGAGCCTCAGTTTCTCTTTTTATAAGTAGGAGTCTGGAGTAAATGATTTCCAATGGCTCTCAT TTCAATACAAAATTTCCGTTTATTAAATGCATGAGCTTCTGTTACTCCAAGACTGAGAAGGAAATTGAACCTGAGAC TCATTGACTGGCAAGATGTCCCCAGAGGCTCTCATTCAGCAATAAAATTCTCACCTTCACCCAGGCCCACTGAGTGT CAGATTTGCATGCACTAGTTCACGTGTGTAAAAAGGAGGATGCTTCTTTCCTTTGTATTCTCACATACCTTTAGGAA AGAACTTAGCACCCTTCCCACACAGCCATCCCAATAACTCATTTCAGTGACTCAACCCTTGACTTTATAAAAGTCTT GGGCAGTATAGAGCAGAGATTAAGAGTACAGATGCTGGAGCCAGACCACCTGAGTGATTAGTGACTCAGTTTCTCTT AGTAATTGTATGACTCAGTTTCTTCATCTGTAAAATGGAGGGTTTTTTAATTAGTTTGTTTTTGAGAAAGGGTCTCA CTCTGTCACCCAAATGGGAGTGTAGTGGCAAAATCTCGGCTCACTGCAACTTGCACTTCCCAGGCTCAAGCGGTCCT CCCACCTCAACATCCTGAGTAGCTGGAACCACAGGTACACACCACCATACCTCGCTAATTTTTTGTATTTTTGGTAG AGATGGGGTTTCACATGTTACACAGGATGGTCTCAGACTCCGGAGCTCAAGCAATCTGCCCACCTCAGCCTTCCAAA GTGCTGGGATTATAAGCATGATTACAGGAGTTTTAACAGGCTCATAAGATTGTTCTGCAGCCCGAGTGAGTTAATAC ATGCAAAGAGTTTAAAGCAGTGACTTATAAATGCTAACTACTCTAGAAATGTTTGCTAGTATTTTTTGTTTAACTGC AATCATTCTTGCTGCAGGTGAAAACTAGTGTTCTGTACTTTATGCCCATTCATCTTTAACTGTAATAATAAAAATAA CTGACATTTATTGAAGGCTATCAGAGACTGTAATTAGTGCTTTGCATAATTAATCATATTTAATACTCTTGGATTCT TTCAGGTAGATACTATTATTATCCCCATTTTACTACAGTTAAAAAAACTACCTCTCAACTTGCTCAAGCATACACTC TCACACACACAAACATAAACTACTAGCAAATAGTAGAATTGAGATTTGGTCCTAATTATGTCTTTGCTCACTATCCA ATAAATATTTATTGACATGTACTTCTTGGCAGTCTGTATGCTGGATGCTGGGGATACAAAGATGTTTAAATTTAAGC TCCAGTCTCTGCTTCCAAAGGCCTCCCAGGCCAAGTTATCCATTCAGAAAGCATTTTTTACTCTTTGCATTCCACTG TTTTTCCTAAGTGACTAAAAAATTACACTTTATTCGTCTGTGTCCTGCTCTGGGATGATAGTCTGACTTTCCTAACC TGAGCCTAACATCCCTGACATCAGGAAAGACTACACCATGTGGAGAAGGGGTGGTGGTTTTGATTGCTGCTGTCTTC AGTTAGATGGTTAACTTTGTGAAGTTGAAAACTGTGGCTCTCTGGTTGACTGTTAGAGTTCTGGCACTTGTCACTAT GCCTATTATTTAACAAATGCATGAATGCTTCAGAATATGGGAATATTATCTTCTGGAATAGGGAATCAAGTTATATT ATGTAACCCAGGATTAGAAGATTCTTCTGTGTGTAAGAATTTCATAAACATTAAGCTGTCTAGCAAAAGCAAGGGCT TGGAAAATCTGTGAGCTCCTCACCATATAGAAAGCTTTTAACCCATCATTGAATAAATCCCTATAGGGGATTTCTAC CCTGAGCAAAAGGCTGGTCTTGATTAATTCCCAAACTCATATAGCTCTGAGAAAGTCTATGCTGTTAACGTTTTCTT GTCTGCTACCCCATCATATGCACAACAATAAATGCAGGCCTAGGCATGACTGAAGGCTCTCTCATAATTCTTGGTTG CATGAATCAGATTATCAACAGAAATGTTGAGACAAACTATGGGGAAGCAGGGTATGAAAGAGCTCTGAATGAAATGG AAACCGCAATGCTTCCTGCCCATTCAGGGCTCCAGCATGTAGAAATCTGGGGCTTTGTGAAGACTGGCTTAAAATCA GAAGCCCCATTGGATAAGAGTAGGGAAGAACCTAGAGCCTACGCTGAGCAGGTTTCCTTCATGTGACAGGGAGCCTC CTGCCCCGAACTTCCAGGGATCCTCTCTTAAGTGTTTCCTGCTGGAATCTCCTCACTTCTATCTGGAAATGGTTTCT CCACAGTCCAGCCCCTGGCTAGTTGAAAGAGTTACCCATGCAGAGGCCCTCCTAGCATCCAGAGACTAGTGCTTAGA TTCCTACTTTCAGCGTTGGACAACCTGGATCCACTTGCCCAGTGTTCTTCCTTAGTTCCTACCTTCGACCTTGATCC TCCTTTATCTTCCTGAACCCTGCTGAGATGATCTATGTGGGGAGAATGGCTTCTTTGAGAAACATCTTCTTCGTTAG TGGCCTGCCCCTCATTCCCACTTTAATATCCAGAATCACTATAAGAAGAATATAATAAGAGGAATAACTCTTATTAT AGGTAAGGGAAAATTAAGAGGCATACGTGATGGGATGAGTAAGAGAGGAGAGGGAAGGATTAATGGATGATAAAATC TACTACTATTTGTTGAGACCTTTTATAGTCTAATCAATTTTGCTATTGTTTTCCATCCTCACGCTAACTCCATAAAA AAACACTATTATTATCTTTATTTTGCCATGACAAGACTGAGCTCAGAAGAGTCAAGCATTTGCCTAAGGTCGGACAT GTCAGAGGCAGTGCCAGACCTATGTGAGACTCTGCAGCTACTGCTCATGGGCCCTGTGCTGCACTGATGAGGAGGAT CAGATGGATGGGGCAATGAAGCAAAGGAATCATTCTGTGGATAAAGGAGACAGCCATGAAGAAGTCTATGACTGTAA ATTTGGGAGCAGGAGTCTCTAAGGACTTGGATTTCAAGGAATTTTGACTCAGCAAACACAAGACCCTCACGGTGACT TTGCGAGCTGGTGTGCCAGATGTGTCTATCAGAGGTTCCAGGGAGGGTGGGGTGGGGTCAGGGCTGGCCACCAGCTA TCAGGGCCCAGATGGGTTATAGGCTGGCAGGCTCAGATAGGTGGTTAGGTCAGGTTGGTGGTGCTGGGTGGAGTCCA TGACTCCCAGGAGCCAGGAGAGATAGACCATGAGTAGAGGGCAGACATGGGAAAGGTGGGGGAGGCACAGCATAGCA GCATTTTTCATTCTACTACTACATGGGACTGCTCCCCTATACCCCCAGCTAGGGGCAAGTGCCTTGACTCCTATGTT TTCAGGATCATCATCTATAAAGTAAGAGTAATAATTGTGTCTATCTCATAGGGTTATTATGAGGATCAAAGGAGATG CACACTCTCTGGACCAGTGGCCTAACAGTTCAGGACAGAGCTATGGGCTTCCTATGTATGGGTCAGTGGTCTCAATG TAGCAGGCAAGTTCCAGAAGATAGCATCAACCACTGTTAGAGATATACTGCCAGTCTCAGAGCCTGATGTTAATTTA GCAATGGGCTGGGACCCTCCTCCAGTAGAACCTTCTAACCAGCTGCTGCAGTCAAAGTCGAATGCAGCTGGTTAGAC TTTTTTTAATGAAAGCTTAGCTTTCATTAAAGATTAAGCTCCTAAGCAGGGCACAGATGAAATTGTCTAACAGCAAC TTTGCCATCTAAAAAAATCTGACTTCACTGGAAACATGGAAGCCCAAGGTTCTGAACATGAGAAATTTTTAGGAATC TGCACAGGAGTTGAGAGGGAAACAAGATGGTGAAGGGACTAGAAACCACATGAGAGACACGAGGAAATAGTGTAGAT TTAGGCTGGAGGTAAATGAAAGAGAAGTGGGAATTAATACTTACTGAAATCTTTCTATATGTCAGGTGCCATTTTAT GATATTTAATAATCTCATTACATATGGTAATTCTGTGAGATATGTATTATTGAACATACTATAATTAATACTAATGA TAAGTAACACCTCTTGAGTACTTAGTATATGCTAGAATCAAATTTAAGTTTATCATATGAGGCCGGGCACGGTGGCT CATATATGGGATTACATGCCTGTAATCCCAGCACTTTGGGAGGCCAAGGCAATTGGATCACCTGAGGTCAGGAGTTC CAGACCAGCCTGGCCAACATGGTGAAACCCCTTCTCTACTAAAAAATACAAAAAATCAGCCAGGTGTGGTGGCACGC GTCTATAATCCCAGCTACTCAGGAGGCTGAGGCAGGAGAATCACTTGAACCCAGGAGGTGGAGGTTGCAGTGAGCTA AGATTGCACCACTGCACTCCAGCCTAGGCGACAGAGTGAGACTCCATCTCAAAAAAAAAAAAAGAAGTTTATTATAT GAATTAACTTAGTTTTACTCACACCAATACTCAGAAGTAGATTATTACCTCATTTATTGATGAGGAGCCCAATGTAC TTGTAGTGTAGATCAACTTATTGAAAGCACAAGCTAATAAGTAGACAATTAGTAATTAGAAGTCAGATGGTCTGAGC TCTCCTACTGTCTACATTACATGAGCTCTTATTAACTGGGGACTCGAAAATCAAAGACATGAAATAATTTGTCCAAG CTTACAGAACCACCAAGTAGTAAGGCTAGGATGTAGACCCAGTTCTGCTACCTCTGAAGACAGTGTTTTTTCCACAG CAAAACACAAACTCAGATATTGTGGATGCGAGAAATTAGAAGTAGATATTCCTGCCCTGTGGCCCTTGCTTCTTACT TTTACTTCTTGGCGATTGGAAGTTGTGGTCCAAGCCACAGTTGCAGACCATACTTCCTCAACCATAATTGCATTTCT TCAGGAAAGTTTGAGGGAGAAAAAGGTAAAGAAAAATTTAGAAACAACTTCAGAATAAAGAGATTTTCTCTTGGGTT ACAGAGATTGTCATATGACAAATTATAAGCAGACACTTGAGAAAACTGAAGGCCCATGCCTGCCCAAATTACCCTTT GACCCCTTGGTCAAGCTGCAACTTTGGTTAAAGGGAGTGTTTATGTGTTATAGTGTTCATTTACTCTTCTGGTCTAA CCCATTGGCTCCGTCTTCATCCTGCAGTGACCTCAGTGCCTCAGAAACATACATATGTTTGTCTAGTTTAAGTTTGT GTGAAATTCTAACTAGCGTCAAGAACTGAGGGCCCTAAACTATGCTAGGAATAGTGCTGTGGTGCTGTGATAGGTAC ACAAGAAATGAGAAGAAACTGCAGATTCTCTGCATCTCCCTTTGCCGGGTCTGACAACAAAGTTTCCCCAAATTTTA CCAATGCAAGCCATTTCTCCATATGCTAACTACTTTAAAATCATTTGGGGCTTCACATTGTCTTTCTCATCTGTAAA AAGAATGGAAGAACTCATTCCTACAGAACTCCCTATGTCTTCCCTGATGGGCTAGAGTTCCTCTTTCTCAAAAATTA GCCATTATTGTATTTCCTTCTAAGCCAAAGCTCAGAGGTCTTGTATTGCCCAGTGACATGCACACTGGTCAAAAGTA GGCTAAGTAGAAGGGTACTTTCACAGGAACAGAGAGCAAAAGAGGTGGGTGAATGAGAGGGTAAGTGAGAAAAGACA AATGAGAAGTTACAACATGATGGCTTGTTGTCTAAATATCTCCTAGGGAATTATTGTGAGAGGTCTGAATAGTGTTG TAAAATAAGCTGAATCTGCTGCCTAACATTAACAGTCAAGAAATACCTCCGAATAACTGTACCTCCAATTATTCTTT AAGGTAGCATGCAACTGTAATAGTTGCATGTATATATTTATCATAATACTGTAACAGAAAACACTTACTGAATATAT ACTGTGTCCCTAGTTCTTTACACAATAAACTAATCTCATCCTCATAATTCTATTAGCTAATACATATTATCATCCTA TATTTCAGAGACTTCAAGAAGTTAAGCAACTTGCTCAAGATCATCTAAGAAGTAGGTGGTATTTCTGGGCTCATTTG GCCCCTCCTAATCTCTCATGGCAACATGGCTGCCTAAAGTGTTGATTGCCTTAATTCATCAGGGATGGGCTCATACT CACTGCAGACCTTAACTGGCATCCTCTTTTCTTATGTGATCTGCCTGACCCTAGTAGAACTTATGAAATTTCTGATG AGAAAGGAGAGAGGAGAAAGGCAGAGCTGACTGTGATGAGTGATGAAGGTGCCTTCTCATCTGGGTACCAGTGGGGC CTCTAAGACTAAGTCACTCTGTCTCACTGTGTCTTAGCCAGTTCCTTACAGCTTGCCCTGATGGGAGATAGAGAATG GGTATCCTCCAACAAAAAAATAAATTTTCATTTCTCAAGGTCCAACTTATGTTTTCTTAATTTTTAAAAAAATCTTG ACCATTCTCCACTCTCTAAAATAATCCACAGTGAGAGAAACATTCTTTTCCCCCATCCCATAAATACCTCTATTAAA TATGGAAAATCTGGGCATGGTGTCTCACACCTGTAATCCCAGCACTTTGGGAGGCTGAGGTGGGTGGACTGCTTGGA GCTCAGGAGTTCAAGACCATCTTGGACAACATGGTGATACCCTGCCTCTACAAAAAGTACAAAAATTAGCCTGGCAT GGTGGTGTGCACCTGTAATCCCAGCTATTAGGGTGGCTGAGGCAGGAGAATTGCTTGAACCCGGGAGGCGGAGGTTG CAGTGAGCTGAGATCGTGCCACTGCACTCCAGCCTGGGGGACAGAGCACATTATAATTAACTGTTATTTTTTACTTG GACTCTTGTGGGGAATAAGATACATGTTTTATTCTTATTTATGATTCAAGCACTGAAAATAGTGTTTAGCATCCAGC AGGTGCTTCAAAACCATTTGCTGAATGATTACTATACTTTTTACAAGCTCAGCTCCCTCTATCCCTTCCAGCATCCT CATCTCTGATTAAATAAGCTTCAGTTTTTCCTTAGTTCCTGTTACATTTCTGTGTGTCTCCATTAGTGACCTCCCAT AGTCCAAGCATGAGCAGTTCTGGCCAGGCCCCTGTCGGGGTCAGTGCCCCACCCCCGCCTTCTGGTTCTGTGTAACC TTCTAAGCAAACCTTCTGGCTCAAGCACAGCAATGCTGAGTCATGATGAGTCATGCTGAGGCTTAGGGTGTGTGCCC AGATGTTCTCAGCCTAGAGTGATGACTCCTATCTGGGTCCCCAGCAGGATGCTTACAGGGCAGATGGCAAAAAAAAG GAGAAGCTGACCACCTGACTAAAACTCCACCTCAAACGGCATCATAAAGAAAATGGATGCCTGAGACAGAATGTGAC ATATTCTAGAATATATTATTTCCTGAATATATATATATATATATATACACATATACGTATATATATATATATATATA TATTTGTTGTTATCAATTGCCATAGAATGATTAGTTATTGTGAATCAAATATTTATCTTGCAGGTGGCCTCTATACC TAGAAGCGGCAGAATCAGGCTTTATTAATACATGTGTATAGATTTTTAGGATCTATACACATGTATTAATATGAAAC AAGGATATGGAAGAGGAAGGCATGAAAACAGGAAAAGAAAACAAACCTTGTTTGCCATTTTAAGGCACCCCTGGACA GCTAGGTGGCAAAAGGCCTGTGCTGTTAGAGGACACATGCTCACATACGGGGTCAGATCTGACTTGGGGTGCTACTG GGAAGCTCTCATCTTAAGGATACATCTCAGGCCAGTCTTGGTGCATTAGGAAGATGTAGGCAACTCTGATCCTGAGA GGAAAGAAACATTCCTCCAGGAGAGCTAAAAGGGTTCACCTGTGTGGGTAACTGTGAAGGACTACAAGAGGATGAAA AACAATGACAGACAGACATAATGCTTGTGGGAGAAAAAACAGGAGGTCAAGGGGATAGAGAAGGCTTCCAGAAGAAT GGCTTTGAAGCTGGCTTCTGTAGGAGTTCACAGTGGCAAAGATGTTTCAGAAATGTGACATGACTTAAGGAACTATA CAAAAAGGAACAAATTTAAGGAGAGGCAGATAAATTAGTTCAACAGACATGCAAGGAATTTTCAGATGAATGTTATG TCTCCACTGAGCTTCTTGAGGTTAGCAGCTGTGAGGGTTTTGCAGGCCCAGGACCCATTACAGGACCTCACGTATAC TTGACACTGTTTTTTGTATTCATTTGTGAATGAATGACCTCTTGTCAGTCTACTCGGTTTCGCTGTGAATGAATGAT GTCTTGTCAGCCTACTTGGTTTCGCTAAGAGCACAGAGAGAAGATTTAGTGATGCTATGTAAAAACTTCCTTTTTGG TTCAAGTGTATGTTTGTGATAGAAATGAAGACAGGCTACATGATGCATATCTAACATAAACACAAACATTAAGAAAG GAAATCAACCTGAAGAGTATTTATACAGATAACAAAATACAGAGAGTGAGTTAAATGTGTAATAACTGTGGCACAGG CTGGAATATGAGCCATTTAAATCACAAATTAATTAGAAAAAAAACAGTGGGGAAAAAATTCCATGGATGGGTCTAGA AAGACTAGCATTGTTTTAGGTTGAGTGGCAGTGTTTAAAGGGTGATATCAGACTAAACTTGAAATATGTGGCTAAAT AACTAGAATACTCTTTATTTTTTCGTATCATGAATAGCAGATATAGCTTGATGGCCCCATGCTTGGTTTAACATCCT TGCTGTTCCTGACATGAAATCCTTAATTTTTGACAAAGGGGCTATTCATTTTCATTTTATATTGGGCCTAGAAATTA TGTAGATGGTCCTGAGGAAAAGTTTATAGCTTGTCTATTTCTCTCTCTAACATAGTTGTCAGCACAATGCCTAGGCT ATAGGAAGTACTCAAAGCTTGTTAAATTGAATTCTATCCTTCTTATTCAATTCTACACATGGAGGAAAAACTCATCA GGGATGGAGGCACGCCTCTAAGGAAGGCAGGTGTGGCTCTGCAGTGTGATTGGGTACTTGCAGGACGAAGGGTGGGG TGGGAGTGGCTAACCTTCCATTCCTAGTGCAGAGGTCACAGCCTAAACATCAAATTCCTTGAGGTGCGGTGGCTCAC TCCTGTAATCACAGCAGTTTGGGACGCCAAGGTGGGCAGATCACTTGAGGTCAGGAGTTGGACACCAGCCCAGCCAA CATAGTGAAACCTGGTCTCTGCTTAAAAATATAAAAATTAGCTGGACGTGGTGACGGGAGCCTGTAATCCAACTACT TGGGAGGCTGAGGCAGGAGAATCGCTTGAACCGGGGAGGTGGAGTTTGCACTGAGCAGAGATCATGCCATTGCACTC CAGCCTCCAGAGCGAGACTCTGTCTAAAGAAAAACGAAAACAAACAAACAAACAAACAAACAAAACCCATCAAATTC CCTGACCGAACAGAATTCTGTCTGATTGTTCTCTGACTTATCTACCATTTTCCCTCCTTAAAGAAACTGTGGAACTT CCTTCAGCTAGAGGGGCCTGGCTCAGAAGCCTCTGGTCAGCATCCAAGAAATACTTGATGTCACTTTGGCTAAAGGT ATGATGTGTAGACAAGCTCCAGAGATGGTTTCTCATTTCCATATCCACCCACCCAGCTTTCCAATTTTAAAGCCAAT TCTGAGGTAGAGACTGTGATGAACAAACACCTTGACAAAATTCAACCCAAAGACTCACTTTGCCTAGCTTCAAAATC CTTACTCTGACATATACTCACAGCCAGAAATTAGCATGCACTAGAGTGTGCATGAGTGCAACACACACACACACCAA TTCCATATTCTCTGTCAGAAAATCCTGTTGGTTTTTCGTGAAAGGATGTTTTCAGAGGCTGACCCCTTGCCTTCACC TCCAATGCTACCACTCTGGTCTAAGTCACTGTCACCACCACCTAAATTATAGCTGTTGACTCATAACAATCTTCCTG CTTCTACCACTGCCCCACTACAATTTCTTCCCAATATACTATCCAAATTAGTCTTTTCAAAATGTAAGTCATATATG GTCACCTCTTTGTTCAAAGTCTTCTGATAGTTTCCTATATCATTTATAATAAAACCAAATCCTTACAATTCTCTACA ATAGTTGTTCATGCATATATTATGTTTATTACAGATACGCATATATATAGCTCTCATATAAATAAATATATATATTT ATGTGTATGTGTGTAGAGTGTTTTTTCTTACAACTCTATGATGTAGGTATTATTAGTGTCCCAAATTTTATAATTTA GGACTTCTATGATCTCATCTTTTATTCTCCCCTTCACCGAATCTCATCCTACATTGGCCTTATTGATATTCCTTGAA AATTCTAAGCATCTTACATCTTTAGGGTATTTACATTTGCCATTCCCTATGCCCTAAATATTTAATCATAGTTTCAT ATAAATGGGTTCCTCATCATCTATGGGTACTCTCTCAGGTGTTAACTTTATAGTGAGGACTTTCCTGCCATACTACT TAAAGTAGCGATACCCTTTCACCCTGTCCTAATCACACTCTGGCCTTCATTTCAGTTTTTTTTTTTTCTCCATAGCA CCTAATCTCATTGGTATATAACATGTTTCATTTGCTTATTTAATGTCAAGCTCTTTCCACTATCAAGTCCATGAAAA CAGGAACTTTATTCCTCTATTCTGTTTTTGTGCTGTATTCTTAGCAATTTTACAATTTTGAATGAAATGAATGAGCA GTCAAACACATATACAACTATAATTAAAAGGATGTATGCTGACACATCCACTGCTATGCACACACAAAGAAATCAGT GGAGTAGAGCTGGAAGCGCTAAGCCTGCATAGAGCTAGTTAGCCCTCCGCAGGCAGAGCCTTGATGGGATTACTGAG TTCTAGAATTGGACTCATTTGTTTTGTAGGCTGAGATTTGCTCTTGAAAACTTGTTCTGACCAAAATAAAAGGCTCA AAAGATGAATATCGAAACCAGGGTGTTTTTTACACTGGAATTTATAACTAGAGCACTCATGTTTATGTAAGCAATTA ATTGTTTCATCAGTCAGGTAAAAGTAAAGAAAAACTGTGCCAAGGCAGGTAGCCTAATGCAATATGCCACTAAAGTA AACATTATTCCATAGGTGTCAGATATGGCTTATTCATCCATCTTCATGGGAAGGATGGCCTTGGCCTGGACATCAGT GTTATGTGAGGTTCAAAACACCTCTAGGCTATAAGGCAACAGAGCTCCTTTTTTTTTTTTCTGTGCTTTCCTGGCTG TCCAAATCTCTAATGATAAGCATACTTCTATTCAATGAGAATATTCTGTAAGATTATAGTTAAGAATTGTGGGAGCC ATTCCGTCTCTTATAGTTAAATTTGAGCTTCTTTTATGATCACTGTTTTTTTAATATGCTTTAAGTTCTGGGGTACA TGTGCCATGGTGGTTTGCTGCACCCATCAACCCGTCATCTACATTAGGTATTTCTCCTAATGCTATCCTTCCCCTAG CCCCCCACCCCCAACAGGCCCCAGTGTGTGATGTTCCCCTCCCTGTGTCCATGGATCACTGGTTTTTTTTTTTTTTT TTTTTTTTTTTTTAAAGTCTCAGTTAAATTTTTGGAATGTAATTTATTTTCCTGGTATCCTAGGACCTGCAAGTTAT CTGGTCACTTTAGCCCTCACGTTTTGATGATAATCACATATTTGTAAACACAACACACACACACACACACACACACA TATATATATATAAAACATATATATACATAAACACACATAACATATTTATCGGGCATTTCTGAGCAACTAACTCATGC AGGACTCTCAAACACTAACCTATAGCCTTTTCTATGTATCTACTTGTGTAGAAACCAAGCGTGGGGACTGAGAAGGC AATAGCAGGAGCATTCTGACTCTCACTGCCTTTGGCTAGGTCCCTCCCTCATCACAGCTCAGCATAGTCCGAGCTCT TATCTATATCCACACACAGTTTCTGACGCTGCCCAGCTATCACCATCCCAAGTCTAAAGAAAAAAATAATGGGTTTG CCCATCTCTGTTGATTAGAAAACAAAACAAAATAAAATAAGCCCCTAAGCTCCCAGAAAACATGACTAAACCAGCAA GAAGAAGAAAATACAATAGGTATATGAGGAGACTGGTGACACTAGTGTCTGAATGAGGCTTGAGTACAGAAAAGAGG CTCTAGCAGCATAGTGGTTTAGAGGAGATGTTTCTTTCCTTCACAGATGCCTTAGCCTCAATAAGCTTGCGGTTGTG GAAGTTTACTTTCAGAACAAACTCCTGTGGGGCTAGAATTATTGATGGCTAAAAGAAGCCCGGGGGAGGGAAAAATC ATTCAGCATCCTCACCCTTAGTGACACAAAACAGAGGGGGCCTGGTTTTCCATATTTCCTCATGATGGATGATCTCG TTAATGAAGGTGGTCTGACGAGATCATTGCTTCTTCCATTTAAGCCTTGCTCACTTGCCAATCCTCAGTTTTAACCT TCTCCAGAGAAATACACATTTTTTATTCAGGAAACATACTATGTTATAGTTTCAATACTAAATAATCAAAGTACTGA AGATAGCATGCATAGGCAAGAAAAAGTCCTTAGCTTTATGTTGCTGTTGTTTCAGAATTTAAAAAAGATCACCAAGT CAAGGACTTCTCAGTTCTAGCACTAGAGGTGGAATCTTAGCATATAATCAGAGGTTTTTCAAAATTTCTAGACATGA GATTCAAAGCCCTGCACTTAAAATAGTCTCATTTGAATTAACTCTTTATATAAATTGAAAGCACATTCTGAACTACT TCAGAGTATTGTTTTATTTCTATGTTCTTAGTTCATAAATACATTAGGCAATGCAATTTAATTAAAAAAACCCAAGA ATTTCTTAGAATTTTAATCATGAAAATAAATGAAGGCATCTTTACTTACTCAAGGTCCCAAAAGGTCAAAGAAACCA GGAAAGTAAAGCTATATTTCAGCGGAAAATGGGATATTTATGAGTTTTCTAAGTTGACAGACTCAAGTTTTAACCTT CAGTGCCCATGATGTAGGAAAGTGTGGCATAACTGGCTGATTCTGGCTTTCTACTCCTTTTTCCCATTAAAGATCCC TCCTGCTTAATTAACATTCACAAGTAACTCTGGTTGTACTTTAGGCACAGTGGCTCCCGAGGTCAGTCACACAATAG GATGTCTGTGCTCCAAGTTGCCAGAGAGAGAGATTACTCTTGAGAATGAGCCTCAGCCCTGGCTCAAACTCACCTGC AAACTTCGTGAGAGATGAGGCAGAGGTACACTACGAAAGCAACAGTTAGAAGCTAAATGATGAGAACACATGGACTC ATAGAGGGAAACAACGCATACTGGGGCCTATCAGAGGGTGGAGGGTGAGAGAAGGAGAGGATCAGGAAAAATCACTA ATGGATGCTAAGCGTAATACCTGAGTGATGAGATCATCTATACAACAAACCCCCTTGACATTCATTTATCTATGTAA CAAACCTGCACATCCTGTACACGTACCCCTGAACTTAAAATAAAAGTTGAAAACAAGAAAGCAACAGTTTGAACACT TGTTATGGTCTATTCTCTCATTCTTTACAATTACACTAGAAAATAGCCACAGGCTCCTGCAAGGCAGCCACAGAATT TATGACTTGTGATATCCAAGTCATTCCTGGATAATGCAAAATCTAACACAAAATCTAGTAGAATCATTTGCTTACAT CTATTTTTGTTCTGAGAATATAGATTTAGATACATAATGGAAGCAGAATAATTTAAAATCTGGCTAATTTAGAATCC TAAGCAGCTCTTTTCCTATCAGTGGTTTACAAGCCTTGTTTATATTTTTCCTATTTTAAAAATAAAAATAAAGTAAG TTATTTGTGGTAAAGAATATTCATTAAAGTATTTATTTCTTAGATAATACCATGAAAAACATTCAGTGAAGTGAAGG GCCTACTTTACCCAACAAGAATCTAATTTATATAATTTTTCATACTAATAGCATCTAAGAACAGTACAATATTTGAC TCTTCAGGTTAAACATATGTCATAAATTAGCCAGAAAGATTTAAGAAAATATTGGATGTTTCCTTGTTTAAATTAGG CATCTTACAGTTTTTAGAATCCTGCATAGAACTTAAGAAATTACAAATGCTAAAGCAAACCCAAACAGGCAGGAATT AATCTTCATCGAATTTGGGTGTTTCTTTCTAAAAGTCCTTTATACTTAAATGTCTTAAGACATACATAGATTTTATT TTACTAATTTTAATTATACAGACAATAAATGAATATTCTTACTGATTACTTTTTCTGACTGTCTAATCTTTCTGATC TATCCTGGATGGCCATAACACTTATCTCTCTGAACTTTGGGCTTTTAATATAGGAAAGAAAAGCAATAATCCATTTT TCATGGTATCTCATATGATAAACAAATAAAATGCTTAAAAATGAGCAGGTGAAGCAATTTATCTTGAACCAACAAGC ATCGAAGCAATAATGAGACTGCCCGCAGCCTACCTGACTTCTGAGTCAGGATTTATAAGCCTTGTTACTGAGACACA AACCTGGGCCTTTCAATGCTATAACCTTTCTTGAAGCTCCTCCCTACCACCTTTAGCCATAAGGAAACATGGAATGG GTCAGATCCCTGGATGCAAGCCAGGTCTGGAACCATAGGCAGTAAGGAGAGAAGAAAATGTGGGCTCTGCAACTGGC TCCGAGGGAGCAGGAGAGAATCAACCCCATACTCTGAATCTAAGAGAAGACTGGTGTCCATACTCTGAATGGGAAGA ATGATGGGATTACCCATAGGGCTTGTTTTAGGGAGAAACCTGTTCTCCAAACTCTTGGCCTTGAGATACCTGGTCCT TATTCCTTGGACTTTGGCAATGTCTGACCCTCACATTCAAGTTCTGAGGAAGGGCCACTGCCTTCATACTGTGGATC TGTAGCAAATTCCCCCTGAAAACCCAGAGCTGTATCTTAATTGTTTAAAAAAATTATATTATCTCAAGGACTGTTCT TCTCTGAGTAGCCAAGCTCAGCTTGGTTCAAGCTACAAGCAGCTGCGCTGCTTTTTGTCTAGTCATTGTTCTTTTAT TTCAGTGGATCAAATACGTTCTTTCCAAACCTAGGATCTTGTCTTCCTGGACTATATATTTTATCCACGAAGTCTTA ATCTGGGGTCCACAGAACACTAGGGGGCTGGTGAAGTTTATAGAAAAAAAATCTGTATTTTTACTTACATGTAACTG AAATTTAGCATTTTCTTCTACTTTGAATGCAAAGGACAAACTAGAATGACATCATCAGTACCTATTGCATAGTTATA AAGAGAAACCACAGATATTTTCATACTACACCATAGGTATTGCAGATCTTTTTGTTTTTGTTTTTGTTTGAGATGGA GTTTCGCTCTTATTGCCCAGGCTGGAGTGCAGTGGCATGATTTCGGCTCACTGCAACCTCCCCTTCCTGCATTCAAG CAATTCTCCTGCCTTGGCCTCCAGAGTAGCTGGGGATTACAGGCACCTGCCACCATGCCAGTCTAATTTTTGTATTT TTAGTAGAGAATGGGTTTCGCCATGTTGGCCAGGCTGGTCTTGAACTCCTGACCTCAGATGATCTGCCCGCCTTGGC CTCCTGAAGTGCTGGGATTATAGGTGTGAGCCACCACGCCTGGCCCATTGCAGATATTTTTAATTCACATTTATCTG CATCACTACTTGGATCTTAAGGTAGCTGCAGACCCAATCCCAGATCTAATGCTTTCATAAAGAAGCAAATATAATAA ATACTATACCACAAATGTAATGTTTGATGTCTGATAATGATATTTCAGTGTAATTAAACTTAGCACTCCATGTATAT TATTTGATGCAATAAAAACATATTTTTTTAGCACTTACAGTCTGCCAAACTGGCCTGTGACACAAAAAAAGTTTAGG GGAATTCCCCTAGTTTTGTCTGTGTTAGCCAATGGTTAGAATATATGCTCAGAAAGATACCATTGGTTAATAGCTAA AAGAAAATGGAGTAGAAATTCAGTGGCCTGGAATAATAACAATTTGGGCAGTCATTAAGTCAGGTGAAGACTTCTGG AATCATGGGAGAAAAGCAAGGGAGACATTCTTACTTGCCACAAGTGTTTTTTTTTTTTTTTTTTTTTATCACAAACA TAAGAAAATATAATAAATAACAAAGTCAGGTTATAGAAGAGAGAAACGCTCTTAGTAAACTTGGAATATGGAATCCC CAAAGGCACTTGACTTGGGAGACAGGAGCCATACTGCTAAGTGAAAAAGACGAAGAACCTCTAGGGCCTGAACATAC AGGAAATTGTAGGAACAGAAATTCCTAGATCTGGTGGGGCAAGGGGAGCCATAGGAGAAAGAAATGGTAGAAATGGA TGGAGACGGAGGCAGAGGTGGGCAGATCATGAGGTCAAGAGATCGAGACCATCCTGGCAAACATGGTGAAATCCCGT CTCTACTAAAAAAAAAAAATTAGCTGGGCATGGTGGCATGCGCCTGTAGTCCCAGCTGCTCGGGAGGCTGAGGCAG GAGAATCGTTTGAACCCAGGAGGCGAAGGTTGCAGTGAGCTGAGATAGTGCCATTGCACTCCAGTCTGGCAACAGAG TGAGACTCCGTCTCAAAAAAAAAAAAAAGAAAGAAAGAAAAGAAAAAGAAAAAAGAAAAAATAAATGGATGTAGAAC AAGCCAGAAGGAGGAACTGGGCTGGGGCAATGAGATTATGGTGATGTAAGGGACTTTTATAGAATTAACAATGCTGG AATTTGTGGAACTCTGCTTCTATTATTCCCCCAATCATTACTTCTGTCACATTGATAGTTAAATAATTTCTGTGAAT TTATTCCTTGANTCCCAAAATATTGAGGTAAATAACAATGGTATTATAAAAGGGCAGATTAAGTGATATAGCATAAG CAATATTCTTCAGGCACATGGATCGAATTGAATACACTGTAAATCCCAACTTCCAGTTTCAGCTCTACCAAGTAAAG AGCTAGCAAGTCATCAAAATGGGGACATACAGAAAAAAAAAAGGACACTAGAGGAATAATATACCCTGACTCCTAGC CTGATTAATATATCGATTCACTTTTACTCTGTTTGGTGACAAATTCTGGCTTTAAATAATTTTAGGATTTTAGGCTT CTCAGCTCCCTTCCCAGTGAGAAGTATAAGCAGGACAGCAGGCAAGCAAGAAGAGAGCCCAAGGCAATACTCACAAA GTAGCCAGTGTCCCCTGTGGTCATAGAGAAATGGAAAGAGAGAGGANTCCCCCCTTGGAGCCACTGGGTGGTAATCC TTTCCGTCCGTTCCTCTCTAGGGAATCACCCCAAGGTACTGTACTTTGGGATTAAGGCTTTAGTCCCACTGTGGACT ACTTGCTATTCTGTTCAGTTTCTGAAGGAACTATGTACGGTTTTTGTCTCCCTAGAGAAACTAAGGTACAGAAGTTT TGTTTACAATGCACTCCTTAAGAGAGCTAGAACTGGGTGAAGANTCCTGGTTTAACCAGCCTTAATTTCCTTTCCCT GGGCCCCGGTTTGGTCACGTCACTGTCACCACCTTTAAGGCAAATGTTAAATGCGCTTTGGCTGAACTTTTTCCTAT TTTGAGATTTGCTCCTTTATATGAGGCTTTCTTGGAAAAGGAGAATGGGAGAGATGGATATCATTTTGGAAGATGAT GAAGAGGGTAAAAAAGGGTACAAATGGAAATTTGTGTTGCAGATAGTATGAGGAGCCAACAAAAAAGAGCCTCAGGA TCCAGCACACATTATCACAAACTTAGTGTCCATCCATCACTGCTGACCCTCTCCGGACCTGACTCCACCCCTGAGGA CACAGGTCAGCCTTGACCAATGACTTTTAAGTACCATGGAGAACAGGGGGCCAGAACTTCGGCAGTAAAGAATAAAA GGCCAGACAGAGAGGCAGCAGCACATATCTGCTTCCGACACAGCTGCAATCACTAGCAAGCTCTCAGGCCTGGCATC ATGGTGCATTTTACTGCTGAGGAGAAGGCTGCCGTCACTAGCCTGTGGAGCAAGATGAATGTGGAAGAGGCTGGAGG TGAAGCCTTGGGCAGGTAAGCATTGGTTCTCAATGCATGGGAATGAAGGGTGAATATTACCCTAGCAAGTTGATTGG GAAAGTCCTCAAGATTTTTTGCATCTCTAATTTTGTATCTGATATGGTGTCATTTCATAGACTCCTCGTTGTTTACC CCTGGACCCAGAGATTTTTTGACAGCTTTGGAAACCTGTCGTCTCCCTCTGCCATCCTGGGCAACCCCAAGGTCAAG GCCCATGGCAAGAAGGTGCTGACTTCCTTTGGAGATGCTATTAAAAACATGGACAACCTCAAGCCCGCCTTTGCTAA GCTGAGTGAGCTGCACTGTGACAAGCTGCATGTGGATCCTGAGAACTTCAAGGTGAGTTCAGGTGCTGGTGATGTGA TTTTTTGGCTTTATATTTTGACATTAATTGAAGCTCATAATCTTATTGGAAAGACCAACAAAGATCTCAGAAATCAT GGGTCGAGCTTGATGTTAGAACAGCAGACTTCTAGTGAGCATAACCAAAACTTACATGATTCAGAACTAGTGACAGT AAAGGACTACTAACAGCCTGAATTGGCTTAACTTTTCAGGAAATCTTGCCAGAACTTGATGTGTTTATCCCAGAGAA TTGTATTATAGAATTGTAGACTTGTGAAAGAAGAATGAAATTTGGCTTTTGGTAGATGAAAGTCCATTTCAAGGAAA TAGAAATGCCTTATTTTATGTGGGTCATGATAATTGAGGTTTAGAAGAGATTTTTGCAAAAAAAATAAAAGATTTGC TCAAAGAAAAATAAGACACATTTTCTAAAATATGTTAAATTTCCCATCAGTATTGTGACCAAGTGAAGGCTTGTTTC CGAATTTGTTGGGGATTTTAAACTCCCGCTGAGAACTCTTGCAGCACTCACATTCTACATTTACAAAAATTAGACAA TTGCTTAAAGAAAAACAGGGAGAGAGGGAACCCAATAATACTGGTAAAATGGGGAAGGGGGTGAGGGTGTAGGTAGG TAGAATGTTGAATGTAGGGCTCATAGAATAAAATTGAACCTAAGCTCATCTGAATTTTTTGGGTGGGCACAAACCTT GGAACAGTTTGAGGTCAGGGTTGTCTAGGAATGTAGGTATAAAGCCGTTTTTGTTTGTTTGTTTGTTTTTTCATCAA GTTGTTTTCGGAAACTTCTACTCAACATGCCTGTGTGTTATTTTGTCTTTTGCCTAACAGCTCCTGGGTAACGTGAT GGTGATTATTCTGGCTACTCACTTTGGCAAGGAGTTCACCCCTGAAGTGCAGGCTGCCTGGCAGAAGCTGGTGTCTG CTGTCGCCATTGCCCTGGCCCATAAGTACCACTGAGTTCTCTTCCAGTTTGCAGGTCTTCCTGTGACCCTGACACCC TCCTTCTGCACATGGGGACTGGGCTTGGCCTTGAGAGAAAGCCTTCTGTTTAATAAAGTACATTTTCTTCAGTAATC AAAAATTGCAATTTTATCTTCTCCATCTTTTACTCTTGTGTTAAAAGGAAAAAGTGTTCATGGGCTGAGGGATGGAG AGAAACATAGGAAGAACCAAGAGCTTCCTTAAGAAATGTATGGGGGCTTGTAAAATTAATGTGGATGTTATGGGAGA ATTCCCAAGATTCCCAAGGAGGATGATATGATGGAGAAAAATCTTTATCGGGGTGGGAAAATGGTTAATTAAGTGGC AGAGACTCCTAGGCAGTTTTTACTGCACCGGGGAAAGAAGGAGCTGTTGTGGTACCTGAGAAAGCAGATTTGTGGTA CATGTCACTTTTCATTAAAAACAAAAACAAAACAAAACAAAACTTCATAGATATCCAAGATATAGGCTGAGAATTAC TATTTTAATTTACTCTTATTTACATTTTGAAGTAGCTAGCTTGTCACATGTTTTATGAAATTGATTTGGAGATAAGA TGAGTGTGTATCAACAATAGCCTGCTCTTTCCATGAAGGATTCCATTATTTCATGGGTTAGCTGAAGCTAAGACACA TGATATCATTGTGCATTATCTTCTGATACAATGTAACATGCACTAAAATAAAGTTAGAGTTAGGACCTGAGTGGGAA AGTTTTTGGAGAGTGTGATGAAGACTTTCCGTGGGAGATAGAATACTAATAAAGGCTTAAATTCTAAAACCAGCAAG CTAGGGCTTCGTGACTTGCATGAAACTGGCTCTCTGGAAGTAGAAGGGAGAGTAAGACATACGTAGAGGACTAGGAA AGACCAGATAGTACAGGGCCTGGCTACAAAAATACAAGCTTTTACTATGCTATTGCAATACTAAACGATAAGCATTA GGATGTTAAGTGACTCAGGAAATAAGATTTTGGGAAAAAGTAATCTGCTTATGTGCACAAAATGGATTCAAGTTTGC AGATAAAATAAAATATGGATGATGATTCAAGGGGACAGATACAATGGTTCAAACCCAAGAGGAGCAGTGAGTCTGTG GAATTTTGAAGGATGGACAAAGGTGGGGTGAGAAAGACATAGTATTCGACCTGACTGTGGGAGATGAGAAGGAAGAA GGAGGTGATAAATGACTGAAAGCTCCCAGACTGGTGAAGATAACAGGAGGAAACCATGCACTTGACCCTGGTGACTC TCATGTGTGAAGGGTAGAGGGATATTAACAGATTTACTTTTTAGGAAGTGCTAGATTGGTCAGGGAGTTTTGACCTT CAGGTCTTGTGTCTTTCATATCAAGGAACCTTTGCATTTTCCAAGTTAGAGTGCCATATTTTGGCAAATATAACTTT ATTAGTAATTTTATAGTGCTCTCACATTGATCAGACTTTTTCCTGTGAATTACTTTTGAATTTGGCTGTATATATCC AGAATATGGGAGAGAGACAAATAATTATTGTAGTTGCAGGCTATCAACAATACTGGTCTCTCTGAGCCTTATAACCT TTCAATATGCCCCATAAACAGAGTAAACAGGGATTATTCATGGCACTAAATATTTTCACCTAGGTCAGTCAACAAAT GGAGGCAATGTGCATTTTTTGATACATATTTTTATATATTTATGGGGCATGTGATACTTACATGCCTAGAACATGTG ACTGATTAAGTCTAGATATTTAGGATATCCATTACTTTGAGCATTTATCATTTCTATGTATTGAGAAAATTTCAAAT CCTCATTTCTGACCATTTTGAAATATATAATAAATAGTAATTAACTATAGTCACCCTACTCAAATATCAACATTATA AACTAACTAATCCTTCTTTCCACTTTTTTACCAACCAACATCTCTTAAATCCCCTGCCATACACATCACACATTTTT CAGCTCTGATAACTATCATTCTACTCTCATACCACCATGAGACCACTTTTTTAGCTCCACAGATGAATAAAAACATG TGATATTTGACTTTCTGTATCTGGCTTATTTTATTATCTATCTCTTTGGCATACCAAGAGTTTGTTTTTGTTCTGCT TCAGGGCTTTCAATTAACATAATGACCTCTGGTTCCATCCATGTTGCTACAAATGACAAGATTTCATTCTTTTTCAT GGCAAAATAGTACTGTGCAAAAAATACAATTTTTTAATCCGTTCATCTGTTGATAGACACTTAGGTTGATCCCAAAC CTTAACTATTGTGAATAGGTGCTTCAATAAACATGAGTGTAATGTGTCCATTGGATATACTGATTTCCTTTCTTTTG GATAAATAACCACTAGTGAGATTGCTGGATTGTATGATAGTTCTGTTTTTAGTTTATTGAGAAATCTTCATACTGTT TTCCATAATGGTTGTACTATTTTACATTCCCACCAACAGTGTGTAAGAAAGAGTTCCCTTTTCTCCATATCCTCACA AGGATCTGTTATTTTTTGTCTTTTTTGTTAATAGCATTTTAACTAGAGTAAGTAGATATCTCATTGTAGTTTTGATT TGCATTTCCCTGATCATTAGTGATGTTGAGATTTTTTCATATGTTTGTTGGTCATTTGTATATCTTTTTCTGAGATT GTCTGTTCATGTCCTTATCCTACTTTTATTGGGATTGTTGTTATTTTCTTGATAATCATTGTGTCATTTTAGAGCCT GGATATTATTCTTTTGTCAGATGTATAGATTGTGAAGATTTTCTCCTCTGTGGGTTGTCTGTTTATTCTGCAGACTC TTCCTTTTGCCATGCAAAAGCTCTTTAGTTTAATTTAGTCCCAGATATTTTCTTTGTTTTTATGTGTTTGCATTTGT GTTCTTGTCATGAAATCCTTTCCTAAGCCAATGTGTAGAAGGGTTTTTCCGATGTTATTTTCTAGAATTGTTACAGT TTCAGGCTTAGATTTAAGTCCTTGATCCATCTTAAGTTGATTTTTGTATAAGGTGAGAGATGAAGATCCAGTTTCAT TCTCCTACATGTAGCTTGCCAGCTATCCCGACTCATTTGTTGAATAGGGTGCCCTTTCCCATTTATGTTTTTGTTTG CTTTGTCAAAGATCAGTTCGGATGTAAGTATTTGAGTTTATTTCTGGGTTCTCTATTCTGTTCCATTGGTCCGATGT GCCTATTTGTACACCAGCATCATGCTGTGTTTTTGGTGACTATGGCCTTATTGTATAGTTTGAAATGAGGTAATGTA ATGCCATTCAGATTTGTTCTTTTTTTTAGACTTGCTTGTTTATTGGGCTCTTTTTTGGTTCCATAAGAATTTTAGGA TTGTTTTTTCTAGTTCTGTGAAGGCTAATGGTGGTATTTATGGGAATTGCAATGCAATTTGTAGGTTGCTTCTGGCA TTATGGCCATTTTCACAATATTGATTCTACCCATCTATGAGAATGGCATGTGTTTCCATTTGTTTGTGTCTTATATG ATTACTATCAGCCGTGTTTTGTAGTTTTCCTTGTAGATGTCTTTCACCTCCTTGGTTAGGTATATATTCCTAAGTTT TTGTTTTGTTTTGTTTTGTTTTTTGCAGCTATTGTAAAAGGGGTTGAGTTATTGATTTTATTCTCATCTTGGTCATT GCTGGTATGTAAGAAAGCAACTCATTGGTGTACGTTAATTTTGTATCCAGAAACTTTGCTGAATTATTTTATCAGTT CTAGGGGGTTTTGGAGGAGTCTTTAGAGTTTTCTACATACACAATCATATCATCAGCAAACAGTGACAGTTTGACTT TCTCTTTAACAATTTGGATGTGCTTTACTTGTTTCTCTTGTCTGATTGCTCTTGCTAGGACTTCCAGTAATATGTTA AAGAGAAGTGGTGAGAGTGGGTATCCTTGTCTCATTCCAGTTTTCAGACAGAATGCTTTTAACTTTTTCCCATTCAA TATAATGTTGGCTGTGTGTTTACCATAGCTGGCTTTTATTACATTGAGGTATGTCCTTTGTAAACCGATTTTGCTGA GTTTTAGTCATAAAGTGATGTTGAATTTTGTTGAATGCAGTTTCTGTGGCTATTGAGATAATCACATGATTTTTGTT TCCAATTCTCTTTATGTTGTGTATCACACTTATTGACTTGCGTATGTTAAACCATCCGTGCATCCCTCGCATGAAAC CACTTGATCATGGGTTTTGATATGCCGTGTGGGATGCTATTAGCTATATTTTGTCAAGGATGTTGGCATCTATGTTC ATCAGGGATATTGATCTGTAGTGTTTTTTTTTTTTGGTTATGTTCTTTCCCAGTTTTGGTATTAAGGTGATACTGGC TTCATAGAATGATTTAGGGAGGATTCTCTCTTTCTCTATCTTGTAGAATACTGTCAATAGGATTGGTATCAATTCTT CTTTGAATGTCTGGTAGAATTCGAACGTCTCCTTTAGGTTTTCTAGTTTATTCATGTAAAGGTGTTCATAGTAACCT TGAATAATCTTTTGTATTTCTGTGGTATCAGTAATAGTATCTCCTGTTTTGTTTCTAACTGAGTTTATTTGCACTTC TCTCCTCTTTTCTTGGTTAATCTTGCTAATGGTCTATCAGTTTTATTTATCTTTTCAAAGAACCAGCTTTTTATTTC ATTTAGCTTTTGTATTTTTTTGCAGTTGTTTTAATTTCATTTAGTTCTCCTCTTATCTTAGTTATTCCCTTTCTTTT GCTGGGTTTTGGTTCTGTTTGTTTTTGTTTCTCTAGTTTCTTGTGGTGTGACCTTATATTGTCTGTCCTCTTTCAGA CTCTTTGACATCGACATTTAGGGCTGTGAACTTTCCTTTTAGCACCATCTTTGCTGTATCCTAGAGGTTTTGATAGG TGTGTCACTATTGTCGGTCAGTTCAAGTAATTTTGTTGTTCTTATTATACTTTAAGTTCTGGGATACATGTGCAGAA TGTGCAGGTTTGTTACATAGGTATAGATGTGCCATGGTGGTTTGCTGCTCCCATCAACCTGTCATCTACATTAGGTA TTTCTTTTAATGTTATCCCTCTCCTAACCCCCTCACCCCCCGACAGGCCCTGGTGTGTGATGTTCCCCTCCCTGTGT CCATGTGTTCTCATTGTTCAACTCCCACTTATGAGTGAGAACGTGTGGTGTTTGGTTTCTCTGTTCCTGTGTTAGTT TGCTCAGAATGATGTTTCCACCTTCACCATGTCCCTGCAAAGACATGAACTCATCATTTTATGGCTGCATATATTCC ATGGTGTATATGTGCCACATTTTCTTTATCCATTATATCGCTGATGGCCATTTGGGTTGGTTCCAAGTCTTTGGTAT TGTGAATAGTGCCGCAATAAACATACGTGTGCACATGTCTTTATAGTAGAATGATTTCTAATTCTTTGGGTATATAC CCAGTAATGGGATTGCTGGGTCAAACAGTATTTCTGGTTCTAGATCCTTGAGGAATTGCCACACTGTCTTCCACAAT GGTTGAACTAATTTACACACCCATCAACAGTGTAAAATTTTTCCTATTCTTCCACATCCTCTCCAGCACCTTTTGTT TCCTGACTTTTTAATAATTGCCATTCTAACTGGCATGAGATGGTATCTCATTGTGGTTTTGATTTGCATTTCTCTAA TGACCAGTGATGATGAGCTTCTTTTCATGTGTTTCTTGGCCACATAAATGACTTCTTTAGAGAAGCATCTGTTCATA TCCTTTGTCCACTTTTTGATGGGGTCGTTAGGTTTTTTCTTGTAAATTTGTTGAAGTTCTTTGTAGATTTTGGATGT TAGCCCTTTGTCAGATGGATAGATTGGCAAAAATTTTCTCCCATTCTGTAGGTTGCCTGTTCACTCTGATGATAGTC TTTTGCTGTGCAGAAGCTCTTTAGTTTAATTAGATCCCATATGTCAATTTTGGCCTTTGTTGTCATTGCTTTTGATG TTTAGTCGTGGAATTTTGCCCATGCCTATGTCCTGAATGGTATTGCCTAGGTTATCTTCTAGGATTTTTATGGTTTT AGGTTGCACATTTAAGTCTTTAATCCACCTTGAGTTAATTTTTGTATAAGGTGTAAGGAAGGGGTACAGTTTCAGTT TTATGCATATTGCTAGCCAGTTTTTCCAGCACCATTTATTAAATAGGGAATTCTTTCTCCATTGCTTTTGTGATGTT TGTCAAAGATCAGATGGTCGTAGATGTGTGGCATTATTTCTGAGGCTTCTGTTCTGTTCCACTGGTCTATATATCTG TTTTGGTACCAGTACCATGCTGTTTTTGTTACTGTAGCCTTGTAGTATAGCTTGAAGTCAGGTAGCATCATGCCTCC AGCTTTGTTCTTTTTGTTTAGGATTGTCTTGGCTATATGGGCTCTTTTTTGATTCCATATGACATTTAAAGTAGTTT TTTCTAATTCTTTGAAAAAAGTCAGTGGTAGCTTGATGGGGATAGCATTGAATCTATAAATTACTTTGGGCAGTATG GCCATTTTAAAGATATTGATTCTTTCTATCTATGAGCATGGAATGTTTTTCCATTTGTTTGTGTCCTCTCTTATTTC CTTGAGCAGTGAGTGGTTTGTAGCTCTCCTTGAAGAGGTTCTTCACATCCCTTATAAGTTGTATTTCTAGGTATTTT ATTTTATTCTCTTTGCAGCAATTGTGAATGGGAGTTCACCCATGATTTGGCTCTCTGCTTGTCTATTATTGGTGTAT AGGAATGCTTGTGATTTTTGCACACTGATTTTGTATCTTGAGACTTTGCTGAAGCTGTTTATCAGCTTAAGATTTTG GGCTGAGATGACAGGGTCTTCTAAATATACAATCATGTCATCTGCAAACAGAGACAATTTGACTTCCTCTCTTCCTA TTTGAATATGCTTTATTTCTTTCTCTTGCCTGATTGTCCTGGCGAGAACTTCCAATACTATGTTGAGTAAGAGTGGC GAGAGGGCATCCTTGTCTTGTGCCGGTTTTCAAAGCAAATGATTTTTAAATTTCCGTCTTGATTTCATTGTTGACCC AATGATCATTCAGGAGCAGGTTATTTAATTTCCCTGTATTTGCATGGTTTTGAAGGTTCCTTTTGTAGTTGATTTCC AATTTTATTCTACTGTGGTCTGAGAGAGTGCTTGATATAATTTCAATTTTTAAAAATTTATTGAGGCTTGTTTTGTG GCATATCATATGGCCTATCTTGGAGAAAGTTCCATGTGCTGATGAATAGAATGTGTATTCTGCAGTTGTTGGGTAGA ATGTCCTGTAAATATCTGTTAAGTCCATTTGTTCTTTAAATCCATTGTTTCTTTGTAGACTGTCTTGATGACCTGCC TAGTGCAGTCAGTGGAGTATTGAAGTCCCCCACTATTATTATGTTGCTGTCTAGTAGTAATTGTTTTATAAATTTGG GATCTCCAGTATTAGATGCATATATATTAAGAATTGTAATATTCTCCCATTGGACAAGGGCTTTTATCATTATATGA TGTCCCTCTTTGTCTTTTTTAACTGCTGTTTCTTTAAAGTTTGTTTTGTCTGACATAAGAATAGCTGCTTTGGCTCG CTTTTGGTGTCCATTTGTGTGGAATGTCATTTTCCACCCCTTTACCTTAAGTTTATGTGAGTCCTTATGTGTTAGGT GAGTCTCCTGAAGGCGGCAGATAACTGGTTGGTGAATTCTATTCATTCTGCAATTCTGTATCTTTTAAGTGGAGCAT TTAGTCCATTTACATTCAACATCAGTATTGAGGTGTGAGGTGACTATTCCATTCTTCGTGGTATTTGTTGCCTGTGT ATCTTTTTATCTGTATTTTTGTTGTATATGTCCTATGGGATTTATGCTTTAAAGAGGTTCTGTTTTGATGTGCTTCC AGGGTTTATTTCAAGATTTAGAGCTCCTTTTATCATTCTTGTAGTGTTGGCTTGGTAGTGCCGAATTCTCTCAGCAT TTGTTTTTCTGAAAAACACTGTGTATTTTCTTCATTTGTGAAGCTTAGTTTCACTGGATATAAAATTCTTGGCTGAT AATTGTTTTGTTTAAGAAGGCTGAAGATAGGGCCATATTCACTTCTAGCTTTTACGGTTTCTGCTGAGAAATCTGCT GTTAATCTGATAGGTTTTCTTTCATAGGTTACCTGGTAGTTTCACCTCACAGCTCTTAAGATTCTCTTTGTCTTTAG ATAACTTTGGATACTCTGATGACAATGTACCTAGGCAATGATATTTTTGCAATGAATTTCCCAGGTGTTTATTGAGC TTCTTTGTATTTGGATATCTAGGTCTCTAGCAAGGAGGGGGAAGTTTTCCTTGATTATTTCCATGGACAAGTTTTCC AAACTTTTAGATTTCTCTTCTTTCTCAGGAATGCTGATTATTCTTAGGTTTGATTGTTTAACATAATCCCAGATTTC TTGGAGGCTTTGTTCATATTTTCTTATTCTTTTTTCTTTGTCTTTGTTGGATTGGGTAATTCAAAAACTTTGTCTTC AAGCTCTGAATTTCTTCTGCTTGGATTCTATTGCTGAGACTTTCTAGAGCATTTTGCATTTCTATAAGTGCATCCAT TCATCCATTGTTTCCTGAAGTTTTGAATGTTTTTTATTTATGCTATCTCTTTAACTGAAGATTTCTCCCCTCATTTC TTGTATCATATTTTTGGTTTTTTTAAAATTGGACTTCACCTTCCTCGGATGCCTCCTTGATTAGCTTAATAACTGAC CTTCTGAATTATTTTTCAGGTAAATCAGGGATTTCTTCTTGGTTTGGATGCATTGCTGGTGAGCTAGTATGATTTTT TGGGGGGTGTTAAAGAACCTTGTTTTTCATATTACCAGAGTTAGTTTTCTGGTTCCTTCTCACTTGGGTAGGCTCTG TCAGAGGGAAAGTCTAGGCCTCAAGGCTGAGACTTTTGTCCCAGCAGGTGTTCCCTTGATGTAGCACAGTCCCCCTT TTCCTAGGACGTGGGGCTTCCTGAGAGCCGAACTGTAGTGATTGTTATCTCTCTTCTGGATCTAGCCACCCATCAGG TCTACCAGACTCCAGGCTGGTACTGGGGTTTGTCTGCACAGAGTCTTGTGACGTGAACCATCTGTGGGTCTCTCAGC CATAGATACAACCACCTGCTCCAATGGAGGTGGTAGAGGATGAAATGAACTCTGTGAGGGTCCTTACTTTTGGTTGT TCAATGCACTATCTTTTTGTGCTGGTTGGCCTCCTGCCAGGAGGTGGCACTTTCTAGAAAGCATCAGCAGAGGCAGT CAGGTGGTGGTGGCTGGGGGGGCTGGGGCACTAGAACTCCCAAGAATATATGCCCTTTGTCTTCAGCTACTAGGGTG AGTAAGGAAGGACCATCAGGTGGGGGCAGGACTAGTCGTGTCTGAGCTCAGAGTCTCCTTGGGCAGGTCTTTCTGTG GCTACTGTGGGAGGATGGGGGTGTAGTTTCCAGGTCAATGGATTTATGTTCCTAGGACAATTATGGCTGCCTCTGCT GTGTCATGCAGGTCATCAGGAAAGTGGGGGAAAGCAAGCAGTCACGTGACTTGCCCAGCTCCCATGCAACTCAAAAG GTTGGTCTCACTTCCAGCGTGCACCCTCCCCCGCAACAGCTCCGAATCTGTTTCCATGCAGTCAGTGAGCAAGGCTG AGAACTTGCCCAGGCTACCAGCTGCGAAACCAAGTAGGGCTGTCCTACTTCCCTGCCAGTGGAGTCTGCACACCAAA TTCATGTCCCCCCACCAACCCCCCCACTGCCCAGCCCCTAGATCTGGCCAGGTGGAGATTTTCTTTTTCCTGTCTCT TTTCCCAGTTCCTCTGGCAGCCCTCCCAAATGACCCCTGTGAGGCAAGGCAGAAATGGCTTCCTAGGGGACCCAGAG AGCCCACAGGGCTTTTCCCGCTGCTTCCTCTACCCCTGTATTTTGCTTGGCCCTCTAAATTGACTCAGCTCCAGGTA AGGTCAGAATCTTCTCCTGTGGTCTAGATCTTCAGGTTCCCAGTGAGGATGTGTGTTTGGGGGTAGACGGTCCCCCT TTTCCACTTCCACAGTTTGGGCACTCACAATATTTGGGGTGTTTCCCGGGTCCTACATGAGCAATCTGCTTCTTTCA GAGGGTGTGTGCGTTCTCTCAGCTTTCTTGAATTTATTTCTGCAGGTGGTTCTGCAAAAAAAATTCCTGATGGGAGA CTTCACATGCTGCTCTGTGCATCCGAGTGGGAGCTGCAATGTACTTCTGCTGCCACCCATCTGCCATCACCCTCTAA TTTGTCGGTAATATGCATTTTTAATCAATCTTTTTTTCTCTCTCTCTCTTTTCTTCTCCCCCAAAACTATACTGCCC TTTGATATCAAGGAATCAAGGCCGTGATGTTGAGGGGTGGGCAGTGGATACACTCTTTACCCCTTAGGGAGCATATC TAGATTTAGATATTGCCAATTCAAGATAACTTAATTGAAAGCAAATTCATAATGAATACACACACACACACACACAT CTGCATGACAAGATTTTTAATAGTTGAAAGAATAACTAATAATTGTCCACAGGCAATAAGGGCTTTTTAAGCAAAAC AGTTGTGATAAAACAGGTCATTCTTAGAATAGTAATCCAGCCAATAGTACAGGTTGCTTAGAGATTATGACATTACC AGAGTTAAAATTCAATAATGGCTTCTCACTCCCTACCACTGAGGACAAGTTTATGTCCTTAGGTTTATGCTTCCCTG AAACAATACCACCTGCTATTCTCCACTTTACATATCAACGGCACTGGTTCTTTATCTAACTCTCTGGCACAGCAGGA GTTTGTTTTCTTCTGCTTCAGAGCTTTGAATTTACTATTTCAGCTTCTAAACTTTATTTGCAATGCCTTCCCATGGC AGACTCCTTCTGTCATTTTGCCTCTGTTCGAAAACTTTTTCCTTAATTTCATTCTTAGTTAATAATATCTGAAATTA TTTTGTTGTTTAACTTAATTATTAATTTTATGTATGTTCTACCTAGATATAATCTTCTAGAGGATTGTTTTATTCTC TGACTTATTTAACTTAAATGCCCACTACCTTTAAAAATTATGACATTTATTTAACAGATATTTGCTGAACAAATGTT TGAAAATACATGGGAAAGAATGCTTGAAAACACTTGAAATTGCTTGTGTAAAGAAACAGTTTTATCAGTTAGGATTT AATCAATGTCAGAAGCAATGATATAGGAAAAATCGAGGAATAAGACAGTTATGGATAAGGAGAAATCAACAAACTCT TAAAAGATATTGCCTCAAAAGCATAAGAGGAAATAAGGGTTTATACATGACTTTTAGAACACTGCCTGGGTTTTTGG ATAAATGGGGAAGTTGTTGGAAAACAGGAGGGATCCTAGATATTCCTTAGTCTGAGGAGGAGCAATTAAGATTCACT TGTTTAGAGGCTGGGAGTGGTGGCTCACGCCTGTAATCCCAGAATTTTGGGAGGCCAAGGCAGGCAGATCACCTGAG GTCAAGAGTTCAAGACCAACCTGGCCAACATGGTGAAATCCCATCTCTACAAAAATACAAAAATTAGACAGGCATGA TGGCAAGTGCCTGTAATCCCAGCTACTTGGGAGGCTGAGGAAGGAGAATTGCTTGAACCTGGAAGGCAGGAGTTGCA GTGAGCCGAGATCATACCACTGCACTCCAGCCTGGGTGACAGAACAAGACTCTGTCTCAAAAAAAAAAAAGAGAGAT TCAAAAGATTCACTTGTTTAGGCCTTAGCGGGCTTAGACACCAGTCTCTGACACATTCTTAAAGGTCAGGCTCTACA AATGGAACCCAACCAGACTCTCAGATATGGCCAAAGATCTATACACACCCATCTCACAGATCCCCTATCTTAAAGAG ACCCTAATTTGGGTTCACCTCAGTCTCTATAATCTGTACCAGCATACCAATAAAAATCTTTCTCACCCATCCTTAGA TTGAGAGAAGTCACTTATTATTATGTGAGTAACTGGAAGATACTGATAAGTTGACAAATCTTTTTCTTTCCTTTCTT ATTCAACTTTTATTTTAACTTCCAAAGAACAAGTGCAATATGTGCAGCTTTGTTGCGCAGGTCAACATGTATCTTTC TGGTCTTTTAGCCGCCTAACACTTTGAGCAGATATAAGCCTTACACAGGATTATGAAGTCTGAAAGGATTCCACCAA TATTATTATAATTCCTATCAACCTGATAAGTTAGGGGAAGGTAGAGCTCTCCTCCAATAAGCCAGATTTCCAGAGTT TCTGACGTCATAATCTACCAAGGTCATGGATCGAGTTCAGAGAAAAAACAAAAGCAAAACCAAACCTACCAAAAAAT AAAAATCCCAAAGAAAAAATAAAGAAAAAAACAGCATGAATACTTCCTGCCATGTTAAGTGGCCAATATGTCAGAAA CAGCACTGAGTTACAGATAAAGATGTCTAAACTACAGTGACATCCCAGCTGTCACAGTGTGTGGACTATTAGTCAAT AAAACAGTCCCTGCCTCTTAAGAGTTGTTTTCCATGCAAATACATGTCTTATGTCTTAGAATAAGATTCCCTAAGAA GTGAACCTAGCATTTATACAAGATAATTAATTCTAATCCATAGTATCTGGTAAAGAGCATTCTACCATCATCTTTAC CGAGCATAGAAGAGCTACACCAAAACCCTGGGTCATCAGCCAGCACATACACTTATCCAGTGATAAATACACATCAT CGGGTGCCTACATACATACCTGAATATAAAAAAAATACTTTTGCTGAGATGAAACAGGCGTGATTTATTTCAAATAG GTACGGATAAGTAGATATTGAAGTAAGGATTCAGTCTTATATTATATTACATAACATTAATCTATTCCTGCACTGAA ACTGTTGCTTTATAGGATTTTTCACTACACTAATGAGAACTTAAGAGATAATGGCCTAAAACCACAGAGAGTATATT CAAGAATAAGTATAGCACTTCTTATTTGGAAACCAATGCTTACTAAATGAGACTAAGACGTGTCCCATCAAAAATCC TGGACCTATGCCTAAAACACATTTCACAATCCCTGAACTTTTCAAAAATTGGTACATGCTTTAACTTTAAACTACAG GCCTCACTGGAGCTACAGACAAGAAGGTGAAAAACGGCTGACAAAAGAAGTCCTGGTATCTTCTATGGTGGGAGAAG AAAACTAGCTAAAGGGAAGAATAAATTAGAGAAAAATTGGAATGACTGAATCGGAACAAGGCAAAGGCTATAAAAAA AATTAAGCAGCAGTATCCTCTTGGGGGCCCCTTCCCCACACTATCTCAATGCAAATATCTGTCTGAAACGGTTCCTG GCTAAACTCCACCCATGGGTTGGCCAGCCTTGCCTTGACCAATAGCCTTGACAAGGCAAACTTGACCAATAGTCTTA GAGTATCCAGTGAGGCCAGGGGCCGGCGGCTGGCTAGGGATGAAGAATAAAAGGAAGCACCCTTCAGCAGTTCCACA CACTCGCTTCTGGAACGTCTGAGGTTATCAATAAGCTCCTAGTCCAGACGCCATGGGTCATTTCACAGAGGAGGACA AGGCTACTATCACAAGCCTGTGGGGCAAGGTGAATGTGGAAGATGCTGGAGGAGAAACCCTGGGAAGGTAGGCTCTG GTGACCAGGACAAGGGAGGGAAGGAAGGACCCTGTGCCTGGCAAAAGTCCAGGTCGCTTCTCAGGATTTGTGGCACC TTCTGACTGTCAAACTGTTCTTGTCAATCTCACAGGCTCCTGGTTGTCTACCCATGGACCCAGAGGTTCTTTGACAG CTTTGGCAACCTGTCCTCTGCCTCTGCCATCATGGGCAACCCCAAAGTCAAGGCACATGGCAAGAAGGTGCTGACTT CCTTGGGAGATGCCATAAAGCACCTGGATGATCTCAAGGGCACCTTTGCCCAGCTGAGTGAACTGCACTGTGACAAG CTGCATGTGGATCCTGAGAACTTCAAGGTGAGTCCAGGAGATGTTTCAGCACTGTTGCCTTTAGTCTCGAGGCAACT TAGACAACTGAGTATTGATCTGAGCACAGCAGGGTGTGAGCTGTTTGAAGATACTGGGGTTGGGAGTGAAGAAACTG CAGAGGACTAACTGGGCTGAGACCCAGTGGCAATGTTTTAGGGCCTAAGGAGTGCCTCTGAAAATCTAGATGGACAA CTTTGACTTTGAGAAAAGAGAGGTGGAAATGAGGAAAATGACTTTTCTTTATTAGATTTCGGTAGAAAGAACTTTCA CCTTTCCCCTATTTTTGTTATTCGTTTTAAAACATCTATCTGGAGGCAGGACAAGTATGGTCGTTAAAAAGATGCAG GCAGAAGGCATATATTGGCTCAGTCAAAGTGGGGAACTTTGGTGGCCAAACATACATTGCTAAGGCTATTCCTATAT CAGCTGGACACATATAAAATGCTGCTAATGCTTCATTACAAACTTATATCCTTTAATTCCAGATGGGGGCAAAGTAT GTCCAGGGGTGAGGAACAATTGAAACATTTGGGCTGGAGTAGATTTTGAAAGTCAGCTCTGTGTGTGTGTGTGTGTG TGTGCGCGCGTGTGTTTGTGTGTGTGTGAGAGCGTGTGTTTCTTTTAACGTTTTCAGCCTACAGCATACAGGGTTCA TGGTGGCAAGAAGATAACAAGATTTAAATTATGGCCAGTGACTAGTGCTGCAAGAAGAACAACTACCTGCATTTAAT GGGAAAGCAAAATCTCAGGCTTTGAGGGAAGTTAACATAGGCTTGATTCTGGGTGGAAGCTTGGTGTGTAGTTATCT GGAGGCCAGGCTGGAGCTCTCAGCTCACTATGGGTTCATCTTTATTGTCTCCTTTCATCTCAACAGCTCCTGGGAAA TGTGCTGGTGACCGTTTTGGCAATCCATTTCGGCAAAGAATTCACCCCTGAGGTGCAGGCTTCCTGGCAGAAGATGG TGACTGGAGTGGCCAGTGCCCTGTCCTCCAGATACCACTGAGCTCACTGCCCATGATGCAGAGCTTTCAAGGATAGG CTTTATTCTGCAAGCAATACAAATAATAAATCTATTCTGCTAAGAGATCACACATGGTTGTCTTCAGTTCTTTTTTT TATGTCTTTTTAAATATATGAGCCACAAAGGGTTTTATGTTGAGGGATGTGTTTATGTGTATTTATACATGGCTATG TGTGTTTGTGTCATGTGCACACTCCACACTTTTTTGTTTACGTTAGATGTGGGTTTTGATGAGCAAATAAAAGAACT AGGCAATAAAGAAACTTATACATGGGAGCGTCTGCAAGTGGGAGTAAAAGGTGCAGGAGAAATCTGGTTGGAAGAAA GACCTCTATAGGACAGGACTCCTCAGAAACAGATGTTTTGGAAGAGATGGGGAAAGGTTCAGTGAAGGGGGCTGAAC CCCCTTCCCTGGATTGCAGCACAGCAGCGAGGAAGGGGCTCAACGAAGAAAAAGTGTTCCAAGCTTTAGGAAGTCAA GGTTTAGGCAGGGATAGCCATTCTATTTTATTAGGGGCAATACTATTTCCAACGGCATCTGGCTTTTCTCAGCCCTT GTGAGGCTCTACGGGGAGGTTGAGGTGTTAGAGATCAGAGCAGGAAACAGGTTTTTCTTTCCACGGTAACTACAATG AAGTGATCCTTACTTTACTAAGGAACTTTTTCATTTTAAGTGTTGACGCATGCCTAAAGAGGTGAAATTAATCCCAT ACCCTTAAGTCTACAGACTGGTCACAGCATTTCAAGGAGGAGACCTCATTGTAAGCTTCTAGGGAGGTGGGGACCTA GGTGAAGGAAATGAGCCAGCAGAAGCTCACAAGTCAGCATCAGCGTGTCATGTCTCAGCAGCAGAACAGCACGGTCA GATGAAAATATAGTGTGAAGAATTTGTATAACATTAATTGAGAAGGCAGATTCACTGGAGTTCTTATATAATTGAAA GTTAATGCACGTTAATAAGCAAGAGTTTAGTTTAATGTGATGGTGTTATGAACTTAACGCTTGTGTCTCCAGAAAAT TCACATGCTGAATCCCCAACTCCCAATTGGCTCCATTTGTGGGGGAGGCTTTGGAAAAGTAATCAGGTTTAGAGGAG CTCATGAGAGCAGATCCCCATCATAGAATTATTTTCCTCATCAGAAGCAGAGAGATTAGCCATTTCTCTTCCTTCTG GTGAGGACACAGTGGGAAGTCAGCCACCTGCAACCCAGGAAGAGAGCCCTGACCAGGAACCAGCAGAAAAGTGAGAA AAAATCCTGTTGTTGAAGTCACCCAGTCTATGCTATTTTGTTATAGCACCTTGCACTAAGTAAGGCAGATGAAGAAA GAGAAAAAAATAAGCTTCGGTGTTCAGTGGATTAGAAACCATGTTTATCTCAGGTTTACAAATCTCCACTTGTCCTC TGTGTTTCAGAATAAAATACCAACTCTACTACTCTCATCTGTAAGATGCAAATAGTAAGCCTGATCCCTTCTGTCTA ACTTCGAATTCTATTTTTTCTTCAACGTACTTTAGGCTTGTAATGTGTTTATATACAGTGAAATGTCAAGTTCTTTC TTTATATTTCTTTCTTTCTTTTTTTTCCTCAGCCTCAGAGTTTTCCACATGCCCTTCCTACCTTCAGGAACTTCTTT CTCCAAACGTCTTCTGCCTGGCCTCCATTCAAATCATAAAGGACCCACTTCAAATGCCATCACTCACTACCATTTCA CAATTCGCACTTTCTTTCTTTGTCCTTTTTTTTTTTAGTAAAACAAGTTTATAAAAAATTGAAGGAATAAATGAATG GCTACTTCATAGGCAGAGTAGACACAAGGGCTACTGGTTGCCGATTTTTATTGTTATTTTTCAATAGTATGCTAAAC AAGGGGTAGATTATTTATGCTGCCCATTTTTAGACCATAAAAGATAACTTCCTGATGTTGCCATGGCATTTTTTTTC CTTTTAATTTTATTTCATTTCATTTTAATTTCGAAGGTACATGTGCAGGATGTGCAGGCTTGTTACATGGGTAAATG TGTGTCTTTCTGGCCTTTTAGCCATCTGTATCAATGAGCAGATATAAGCTTTACACAGGATCATGAAGGATGAAAGA ATTTCACCAATATTATAATAATTTCAATCAACCTGATAGCTTAGGGGATAAACTAATTTGAAGATACAGCTTGCCTC CGATAAGCCAGAATTCCAGAGCTTCTGGCATTATAATCTAGCAAGGTTAGAGATCATGGATCACTTTCAGAGAAAAA CAAAAACAAACTAACCAAAAGCAAAACAGAACCAAAAAACCTCCATAAATACTTCCTACCCAGTTAATGGTCCAATA TGTCAGAAACAGCACTGTGTTAGAAATAAAGCTGTCTAAAGTACACTAATATTCGAGTTATAATAGTGTGTGGACTA TTAGTCAATAAAAACAACCCTTGCCTCTTTAGAGTTGTTTTCCATGTACACGCACATCTTATGTCTTAGAGTAAGAT TCCCTGAGAAGTGAACCTAGCATTTATACAAGATAATTAATTCTAATCCACAGTACCTGCCAAAGAACATTCTACCA TCATCTTTACTGAGCATAGAAGAGCTACGCCAAAACCCTGGGTCATCAGCCAGCACACACACTTATCCAGTGGTAAA TACACATCATCTGGTGTATACATACATACCTGAATATGGAATCAAATATTTTTCTAAGATGAAACAGTCATGATTTA TTTCAAATAGGTACGGATAAGTAGATATTGAGGTAAGCATTAGGTCTTATATTATGTAACACTAATCTATTACTGCG CTGAAACTGTGGTCTTTATGAAAATTGTTTTCACTACACTATTGAGAAATTAAGAGATAATGGCAAAAGTCACAAAG AGTATATTCAAAAAGAAGTATAGCACTTTTTCCTTAGAAACCACTGCTAACTGAAAGAGACTAAGATTTGTCCCGTC AAAAATCCTGGACCTATGCCTAAAACACATTTCACAATCCCTGAACTTTTCAAAAATTGGTACATGCTTTAGCTTTA AACTACAGGCCTCACTGGAGCTACAGACAAGAAGGTAAAAAACGGCTGACAAAAGAAGTCCTGGTATCCTCTATGAT GGGAGAAGGAAACTAGCTAAAGGGAAGAATAAATTAGAGAAAAACTGGAATGACTGAATCGGAACAAGGCAAAGGCT ATAAAAAAAATTAAGCAGCAGTATCCTCTTGGGGGCCCCTTCCCCACACTATCTCAATGCAAATATCTGTCTGAAAC GGTCCCTGGCTAAACTCCACCCATGGGTTGGCCAGCCTTGCCTTGACCAATAGCCTTGACAAGGCAAACTTGACCAA TAGTCTTAGAGTATCCAGTGAGGCCAGGGGCCGGCGGCTGGCTAGGGATGAAGAATAAAAGGAAGCACCCTTCAGCA GTTCCACACACTCGCTTCTGGAACGTCTGAGATTATCAATAAGCTCCTAGTCCAGACGCCATGGGTCATTTCACAGA GGAGGACAAGGCTACTATCACAAGCCTGTGGGGCAAGGTGAATGTGGAAGATGCTGGAGGAGAAACCCTGGGAAGGT AGGCTCTGGTGACCAGGACAAGGGAGGGAAGGAAGGACCCTGTGCCTGGCAAAAGTCCAGGTCGCTTCTCAGGATTT GTGGCACCTTCTGACTGTCAAACTGTTCTTGTCAATCTCACAGGCTCCTGGTTGTCTACCCATGGACCCAGAGGTTC TTTGACAGCTTTGGCAACCTGTCCTCTGCCTCTGCCATCATGGGCAACCCCAAAGTCAAGGCACATGGCAAGAAGGT GCTGACTTCCTTGGGAGATGCCATAAAGCACCTGGATGATCTCAAGGGCACCTTTGCCCAGCTGAGTGAACTGCACT GTGACAAGCTGCATGTGGATCCTGAGAACTTCAAGGTGAGTCCAGGAGATGTTTCAGCACTGTTGCCTTTAGTCTCG AGGCAACTTAGACAACTGAGTATTGATCTGAGCACAGCAGGGTGTGAGCTGTTTGAAGATACTGGGGTTGGGAGTGA AGAAACTGCAGAGGACTAACTGGGCTGAGACCCAGTGGCAATGTTTTAGGGCCTAAGGAGTGCCTCTGAAAATCTAG ATGGACAACTTTGACTTTGAGAAAAGAGAGGTGGAAATGAGGAAAATGACTTTTCTTTATTAGATTTCGGTAGAAAG AACTTTCACCTTTCCCCTATTTTTGTTATTCGTTTTAAAACATCTATCTGGAGGCAGGACAAGTATGGTCGTTAAAA AGATGCAGGCAGAAGGCATATATTGGCTCAGTCAAAGTGGGGAACTTTGGTGGCCAAACATACATTGCTAAGGCTAT TCCTATATCAGCTGGACACATATAAAATGCTGCTAATGCTTCATTACAAACTTATATCCTTTAATTCCAGATGGGGG CAAAGTATGTCCAGGGGTGAGGAACAATTGAAACATTTGGGCTGGAGTAGATTTTGAAAGTCAGCTCTGTGTGTGTG TGTGTGTGTGTGTGTGTCAGCGTGTGTTTCTTTTAACGTCTTCAGCCTACAACATACAGGGTTCATGGTGGGAAGAA GATAGCAAGATTTAAATTATGGCCAGTGACTAGTGCTTGAAGGGGAACAACTACCTGCATTTAATGGGAAGGCAAAA TCTCAGGCTTTGAGGGAAGTTAACATAGGCTTGATTCTGGGTGGAAGCTGGGTGTGTAGTTATCTGGAGGCCAGGCT GGAGCTCTCAGCTCACTATGGGTTCATCTTTATTGTCTCCTTTCATCTCAACAGCTCCTGGGAAATGTGCTGGTGAC CGTTTTGGCAATCCATTTCGGCAAAGAATTCACCCCTGAGGTGCAGGCTTCCTGGCAGAAGATGGTGACTGCAGTGG CCAGTGCCCTGTCCTCCAGATACCACTGAGCCTCTTGCCCATGATTCAGAGCTTTCAAGGATAGGCTTTATTCTGCA AGCAATACAAATAATAAATCTATTCTGCTGAGAGATCACACATGATTTTCTTCAGCTCTTTTTTTTACATCTTTTTA AATATATGAGCCACAAAGGGTTTATATTGAGGGAAGTGTGTATGTGTATTTCTGCATGCCTGTTTGTGTTTGTGGTG TGTGCATGCTCCTCATTTATTTTTATATGAGATGTGCATTTTGATGAGCAAATAAAAGCAGTAAAGACACTTGTACA CGGGAGTTCTGCAAGTGGGAGTAAATGGTGTTGGAGAAATCCGGTGGGAAGAAAGACCTCTATAGGACAGGACTTCT CAGAAACAGATGTTTTGGAAGAGATGGGAAAAGGTTCAGTGAAGACCTGGGGGCTGGATTGATTGCAGCTGAGTAGC AAGGATGGTTCTTAATGAAGGGAAAGTGTTCCAAGCTTTAGGAATTCAAGGTTTAGTCAGGTGTAGCAATTCTATTT TATTAGGAGGAATACTATTTCTAATGGCACTTAGCTTTTCACAGCCCTTGTGGATGCCTAAGAAAGTGAAATTAATC CCATGCCCTCAAGTGTGCAGATTGGTCACAGCATTTCAAGGGAGAGACCTCATTGTAAGACTCTGGGGGAGGTGGGG ACTTAGGTGTAAGAAATGAATCAGCAGAGGCTCACAAGTCAGCATGAGCATGTTATGTCTGAGAAACAGACCAGCAC TGTGAGATCAAAATGTAGTGGGAAGAATTTGTACAACATTAATTGGAAGGTTTACTTAATGGAATTTTTGTATAGTT GGATGTTAGTGCATCTCTATAAGTAAGAGTTTAATATGATGGTGTTACGGACCTGGTGTTTGTGTCTCCTCAAAATT CACATGCTGAATCCCCAACTCCCAACTGACCTTATCTGTGGGGGAGGCTTTTGAAAAGTAATTAGGTTTAGCTGAGC TCATAAGAGCAGATCCCCATCATAAAATTATTTTCCTTATCAGAAGCAGAGAGACAAGCCATTTCTCTTTCCTCCCG GTGAGGACACAGTGAGAAGTCCGCCATCTGCAATCCAGGAAGAGAACCCTGACCACGAGTCAGCCTTCAGAAATGTG AGAAAAAACTCTGTTGTTGAAGCCACCCAGTCTTTTGTATTTTGTTATAGCACCTTACACTGAGTAAGGCAGATGAA GAAGGAGAAAAAAATAAGCTTGGGTTTTGAGTGAACTACAGACCATGTTATCTCAGGTTTGCAAAGCTCCCCTCGTC CCCTATGTTTCAGCATAAAATACCTACTCTACTACTCTCATCTATAAGACCCAAATAATAAGCCTGCGCCCTTCTCT CTAACTTTGATTTCTCCTATTTTTACTTCAACATGCTTTACTCTAGCCTTGTAATGTCTTTACATACAGTGAAATGT AAAGTTCTTTATTCTTTTTTTCTTTCTTTCTTTTTTCTCCTCAGCCTCAGAATTTGGCACATGCCCTTCCTTCTTTC AGGAACTTCTCCAACATCTCTGCCTGGCTCCATCATATCATAAAGGTCCCACTTCAAATGCAGTCACTACCGTTTCA GGATATGCACTTTCTTTCTTTTTTGTTTTTTGTTTTTTTTAAGTCAAAGCAAATTTCTTGAGAGAGTAAAGAAATAA ACGAATGACTACTGCATAGGCAGAGCAGCCCCGAGGGCCGCTGGTTGTTCCTTTTATGGTTATTTCTTGATGATATG TTAAACAAGTTTTGGATTATTTATGCCTTCTCTTTTTAGGCCATATAGGGTAACTTTCTGACATTGCCATGGCATGT TTCTTTTAATTTAATTTACTGTTACCTTAAATTCAGGGGTACACGTACAGGATATGCAGGTTTGTTTTATAGGTAAA AGTGTGCCATGGTTTTAATGGGTTTTTTTTTTCTTGTAAAGTTGTTTAAGTTTCTTGTTTACTCTGGATATTGGCCT TTGTCAGAAGAATAGATTGGAAAATCTTTTTCCCATTCTGTAGATTGTCTTTCGCTCTGATGGTAGTTTCTTTTGCT GAGCAGGAGCTCTTTAGTTTAATTAGATTCCATTGGTCAATTTTTGCTTTTGCTGCAATTGCTTTTCACGCTTTCAT CATGAAATCTGTGCCCGTGTTTATATCATGAATAGTATTGCCTTGATTTTTTTCTAGGCTTTTTATAGTTTGGGGTT TTTCATTTAAGTCTCTAATCCATCCGGAGTTAATTTTGGATAAGGTATAAGGAAGGAGTCCAGTTTCATTTTTCAGC ATATGGCTAGCCAGTTCTCCCCCATCATTTATTAAATTGAAAATCCTTTCCCCATTGCTTGCTTTTGTCAGGTTTCT AAAAGACAGATGGTTGTAGGTACAATATGCAGTTTCTTCAAGTCATATAATACCATCTGAAATCTCTTATTAATTCA TTTCTTTTAGTATGTATGCTGGTCTCCTCTGCTCACTATAGTGAGGGCACCATTAGCCAGAGAATCTGTCTGTCTAG TTCATGTAAGATTCTCAGAATTAAGAAAAATGGATGGCATATGAATGAAACTTCATGGATGACATATGGAATCTAAT GTGTATTTGTTGAATTAATGCATAAGATGCAACAAGGGAAAGGTTGACAACTGCAGTGATAACCTGGTATTGATGAT ATAAGAGTCTATAGATCACAGTAGAAGCAATAATCATGGAAAACAATTGGAAATGGGGAACAGCCACAAACAAGAAA GAATCAATACTACCAGGAAAGTGACTGCAGGTCACTTTTCCTGGAGCGGGTGAGAGAAAAGTGGAAGTTGCAGTAAC TGCCGAATTCCTGGTTGGCTGATGGAAAGATGGGGCAACTGTTCACTGGTACGCAGGGTTTTAGATGTATGTACCTA AGGATATGAGGTATGGCAATGAACAGAAATTCTTTTGGGAATGAGTTTTAGGGCCATTAAAGGACATGACCTGAAGT TTCCTCTGAGGCCAGTCCCCACAACTCAATATAAATGTGTTTCCTGCATATAGTCAAAGTTGCCACTTCTTTTTCTT CATATCATCGATCTCTGCTCTTAAAGATAATCTTGGTTTTGCCTCAAACTGTTTGTCACTACAAACTTTCCCCATGT TCCTAAGTAAAACAGGTAACTGCCTCTCAACTATATCAAGTAGACTAAAATATTGTGTCTCTAATATCAGAAATTCA GCTTTAATATATTGGGTTTAACTCTTTGAAATTTAGAGTCTCCTTGAAATACACATGGGGGTGATTTCCTAAACTTT ATTTCTTGTAAGGATTTATCTCAGGGGTAACACACAAACCAGCATCCTGAACCTCTAAGTATGAGGACAGTAAGCCT TAAGAATATAAAATAAACTGTTCTTCTCTCTGCCGGTGGAAGTGTGCCCTGTCTATTCCTGAAATTGCTTGTTTGAG ACGCATGAGACGTGCAGCACATGAGACACGTGCAGCAGCCTGTGGAATATTGTCAGTGAAGAATGTCTTTGCCTGAT TAGATATAAAGACAAGTTAAACACAGCATTAGACTATAGATCAAGCCTGTGCCAGACACAAATGACCTAATGCCCAG CACGGGCCACGGAATCTCCTATCCTCTTGCTTGAACAGAGCAGCACACTTCTCCCCCAACACTATTAGATGTTCTGG CATAATTTTGTAGATATGTAGGATTTGACATGGACTATTGTTCAATGATTCAGAGGAAATCTCCTTTGTTCAGATAA GTACACTGACTACTAAATGGATTAAAAAACACAGTAATAAAACCCAGTTTTCCCCTTACTTCCCTAGTTTGTTTCTT ATTCTGCTTTCTTCCAAGTTGATGCTGGATAGAGGTGTTTATTTCTATTCTAAAAAGTGATGAAATTGGCCGGGCGC GGTGGCTCACACCTGTAATCCCAGCACTTTGGGAGGCTGAGGTGGGCGGATCACGAGGTCAGGAGATCAAGACCATC CTGGCTAACATGGTGAAACCCCATCTCTACTAAAAATACAAAAAATTAGCCAGAGACGGTGGCGGGTGCCTGTAGTC CCAGCTACTCGGGAGGCTGAGGCAGGAGAATGGCGTGAACCTGGGAGGCAGAGCTGCAGTGAGCAGAGATCGCGCCA CTGCACACTCCAGCCTGGGTGACAAAGCGAGACTCCATCTCAAAAAAAAAAAAAAAAAAAAAAAGAAAGAAAGAAAG AAAAAAAAAGTGATGAAATTGTGTATTCAATGTAGTCTCAAGAGAATTGAAAACCAAGAAAGGCTGTGGCTTCTTCC ACATAAAGCCTGGATGAATAACAGGATAACACGTTGTTACATTGTCACAACTCCTGATCCAGGAATTGATGGCTAAG ATATTCGTAATTCTTATCCTTTTCAGTTGTAACTTATTCCTATTTGTCAGCATTCAGGTTATTAGCGGCTGCTGGCG AAGTCCTTGAGAAATAAACTGCACACTGGATGGTGGGGGTAGTGTAGGAAAATGGAGGGGAAGGAAGTAAAGTTTCA AATTAAGCCTGAACAGCAAAGTTCCCCTGAGAAGGCCACCTGGATTCTATCAGAAACTCGAATGTCCATCTTGCAAA ACTTCCTTGCCCAAACCCCACCCCTGGAGTCACAACCCACCCTTGACCAATAGATTCATTTCACTGAGGGAGGCAAA GGGCTGGTCAATAGATTCATTTCACTGGGAGAGGCAAAGGGCTGGGGGCCAGAGAGGAGAAGTAAAAAGCCACACAT GAAGCAGCAATGCAGGCATGCTTCTGGCTCATCTGTGATCACCAGGAAACTCCCAGATCTGACACTGTAGTGCATTT CACTGCTGACAAGAAGGCTGCTGCCACCAGCCTGTGAAGCAAGGTTAAGGTGAGAAGGCTGGAGGTGAGATTCTGGG CAGGTAGGTACTGGAAGCCGGGACAAGGTGCAGAAAGGCAGAAAGTGTTTCTGAAAGAGGGATTAGCCCGTTGTCTT ACATAGTCTGACTTTGCACCTGCTCTGTGATTATGACTATCCCACAGTCTCCTGGTTGTCTACCCATGGACCTAGAG GTACTTTGAAAGTTTTGGATATCTGGGCTCTGACTGTGCAATAATGGGCAACCCCAAAGTCAAGGCACATGGCAAGA AGGTGCTGATCTCCTTCGGAAAAGCTGTTATGCTCACGGATGACCTCAAAGGCACCTTTGCTACACTGAGTGACCTG CACTGTAACAAGCTGCACGTGGACCCTGAGAACTTCCTGGTGAGTAGTAAGTACACTCACGCTTTCTTCTTTACCCT TAGATATTTGCACTATGGGTACTTTTGAAAGCAGAGGTGGCTTTCTCTTGTGTTATGAGTCAGCTATGGGATATGAT ATTTCAGCAGTGGGATTTTGAGAGTTATGTTGCTGTAAATAACATAACTAAAATTTGGTAGAGCAAGGACTATGAAT AATGGAAGGCCACTTACCATTTGATAGCTCTGAAAAACACATCTTATAAAAAATTCTGGCCAAAATCAAACTGAGTG TTTTGGATGAGGGAACAGAAGTTGAGATAGAGAAAATAACATCTTTCCTTTGGTCAGCGAAATTTTCTATAAAAATT AATAGTCACTTTTCTGCATAGTCCTGGAGGTTAGAAAAAGATCAACTGAACAAAGTAGTGGGAAGCTGTTAAAAGAG GATTGTTTCCCTCCGAATGATGATGGTATACTTTTGTACGCATGGTACAGGATTCTTTGTTATGAGTGTTTGGGAAA ATTGTATGTATGTATGTATGTATGTGATGACTGGGGACTTATCCTATCCATTACTGTTCCTTGAAGTACTATTATCC TACTTTTTAAAAGGACGAAGTCTCTAAAAAAAAAATGAAACAATCACAATATGTTGGGGTAGTGAGTTGGCATAGCA AGTAAGAGAAGGATAGGACACAATGGGAGGTGCAGGGCTGCCAGTCATATTGAAGCTGATATCTAGCCCATAATGGT GAGAGTTGCTCAAACTCTGGTCAAAAAGGATGTAAGTGTTATATCTATTTACTGCAAGTCCAGCTTGAGGCCTTCTA TTCACTATGTACCATTTTCTTTTTTATCTTCACTCCCTCCCCAGCTCTTAGGCAACGTGATATTGATTGTTTTGGCA ACCCACTTCAGCGAGGATTTTACCCTACAGATACAGGCTTCTTGGCAGTAACTAACAAATGCTGTGGTTAATGCTGT AGCCCACAAGACCACTGAGTTCCCTGTCCACTATGTTTGTACCTATGTCCCAAAATCTCATCTCCTTTAGATGGGGG AGGTTGGGGAGAAGAGCAGTATCCTGCCTGCTGATTCAGTTCCTGCATGATAAAAATAGAATAAAGAAATATGCTCT CTAAGAAATATCATTGTACTCTTTTTCTGTCTTTATATTTTACCCTGATTCAGCCAAAAGGACGCACTATTTCTGAT GGAAATGAGAATGTTGGAGAATGGGAGTTTAAGGACAGAGAAGATACTTTCTTGCAATCCTGCAAGAAAAGAGAGAA CTCGTGGGTGGATTTAGTGGGGTAGTTACTCCTAGGAAGGGGAAATCGTCTCTAGAATAAGACAATGTTTTTACAGA AAGGGAGGTCAATGGAGGTACTCTTTGGAGGTGTAAGAGGATTGTTGGTAGTGTGTAGAGGTATGTTAGGACTCAAA TTAGAAGTTCTGTATAGGCTATTATTTGTATGAAACTCAGGATATAGCTCATTTGGTGACTGCAGTTCACTTCTACT TATTTTAAACAACATATTTTTTATGATTTATAATGAAGTGGGGATGGGGCTTCCTAGAGACCAATCAAGGGCCAAAC CTTGAACTTTCTCTTAACGTCTTCAATGGTATTAATAGAGAATTATCTCTAAGGCATGTGAACTGGCTGTCTTGGTT TTCATCTGTACTTCATCTGCTACCTCTGTGACCTGAAACATATTTATAATTCCATTAAGCTGTGCATATGATAGATT TATCATATGTATTTTCCTTAAAGGATTTTTGTAAGAACTAATTGAATTGATACCTGTAAAGTCTTTATCACACTACC CAATAAATAATAAATCTCTTTGTTCAGCTCTCTGTTTCTATAAATATGTACAAGTTTTATTGTTTTTAGTGGTAGTG ATTTTATTCTCTTTCTATATATATACACACACATGTGTGCATTCATAAATATATACAATTTTTATGAATAAAAAATT ATTAGCAATCAATATTGAAAACCACTGATTTTTGTTTATGTGAGCAAACAGCAGATTAAAAGGCTGAGATTTAGGAA ACAGCACGTTAAGTCAAGTTGATAGAGGAGAATATGGACATTTAAAAGAGGCAGGATGATATAAAATTAGGGAAACT GGATGCAGAGACCAGATGAAGTAAGAAAAATAGCTATCGTTTTGAGCAAAAATCACTGAAGTTTCTTGCATATGAGA GTGACATAATAAATAGGGAAACGTAGAAAATTGATTCACATGTATATATATATATAGAACTGATTAGACAAAGTCTA ACTTGGGTATAGTCAGAGGAGCTTGCTGTAATTATATTGAGGTGATGGATAAAGAACTGAAGTTGATGGAAACAATG AAGTTAAGAAAAAAAATCGAGTAAGAGACCATTGTGGCAGTGATTGCACAGAACTGGAAAACATTGTGAAACAGAGA GTCAGAGATGACAGCTAAAATCCCTGTCTGTGAATGAAAAGAAGGAAATTTATTGACAGAACAGCAAATGCCTACAA GCCCCCTGTTTGGATCTGGCAATGAACGTAGCCATTCTGTGGCAATCACTTCAAACTCCTGTACCCAAGACCCTTAG GAAGTATGTAGCACCCTCAAACCTAAAACCTCAAAGAAAGAGGTTTTAGAAGATATAATACCCTTTCTTCTCCAGTT TCATTAATCCCAAAACCTCTTTCTCAAAGTATTTCCTCTATGTGTCCACCCCAAAGAGCTCACCTCACCATATCTCT TGAGTGGGAGCACATAGATAGGCGGTGCTACCATCTAACAGCTTCTGAAATTCCTTTGTCATATTTTTGAGTCCCCA CTAATAACCCACAAAGCAGAATAAATACCAGTTGCTCATGTACAATAATCACTCAACTGCTGTCTTGTAGCATACAT TAATTAAGCACATTCTTTGAATAATTACTGTGTCCAAACAATCACACTTTAAAATCTCACACTTGTGCTATCCCTTG CCCTTCTGAATGTCACTCTGTATTTTAAATGAAGAGATGAGGGTTGAATTTCCTGTGTTACTTATTGTTCATTTCTC GATGAGGAGTTTTCACATTCACCTTTACTGGAAAACACATAAGTACACATCTTACAGGAAAAATATACCAAACTGAC ATGTAGCATGAATGCTTGTGCATGTAGTCATATAAAATCTTGTAGCAATGTAAACATTCTCTGATATACACATACAG ATGTGTCTATATGTCTACACAATTTCTTATGCTCCATGAACAAACATTCCATGCACACATAAGAACACACACTGTTA CAGATGCATACTTGAGTGCATTGACAAAATTACCCCAGTCAATCTAGAGAATTTGGATTTCTGCATTTGACTCTGTT AGCTTTGTACATGCTGTTCATTTACTCTGGGTGATGTCTTTCCCTCATTTTGCCTTGTCTATCTTGTACTCATACTT TAAGTCCTAACTTATATGTTATCTCAACTAAGAAGCTATTTTTTTTTAATTTTAACTGGGCTTAAAGCCCTGTCTAT AAACTCTGCTACAATTATGGGCTCTTTCTTATAATATTTAGTGTTTTTCCTACTAATGTACTTAATCTGCTCATTGT ATATTCCTACCACTAAATTTTAACCTCTTTTATGGTAGAGACATTGTCTTGTAAACTCTTATTTCCCTAGTATTTGG AGATGAAAAAAAAGATTAAATTATCCAAAATTAGATCTCTCTTTTCTACATTATGAGTATTACACTATCCATAGGGA AGTTTGTTTGAGACCTAAACTGAGGAACCTTTGGTTCTAAAATGACTATGTGATATCTTAGTATTTATAGGTCATGA GGTTCCTTCCTCTGCCTCTGCTATAGTTTGATTAGTCAGCAAGCATGTGTCATGCATTTATTCACATCAGAATTTCA TACACTAATAAGACATAGTATCAGAAGTCAGTTTATTAGTTATATCAGTTAGGGTCCATCAAGGAAAGGACAAACCA TTATCAGTTACTCAACCTAGAATTAAATACAGCTCTTAATAGTTAATTATCCTTGTATTGGAAGAGCTAAAATATCA AATAAAGGACAGTGCAGAAATCTAGATGTTAGTAACATCAGAAAACCTCTTCCGCCATTAGGCCTAGAAGGGCAGAA GGAGAAAATGTTTATACCACCAGAGTCCAGAACCAGAGCCCATAACCAGAGGTCCACTGGATTCAGTGAGCTAGTGG GTGCTCCTTGGAGAGAGCCAGAACTGTCTAATGGGGGCATCAAAGTATCAGCCATAAAAAACCATAAAAAAGACTGT CTGCTGTAGGAGATCCGTTCAGAGAGAGAGAGAGACCAGAAATAATCTTGCTTATGCTTTCCCTCAGCCAGTGTTTA CCATTGCAGAATGTACATGCGACTGAAAGGGTGAGGAAACCTGGGAAATGTCAGTTCCTCAAATACAGAGAACACTG AGGGAAGGATGAGAAATAAATGTGAAAGCAGACATGAATGGTAATTGACAGAAGGAAACTAGGATGTGTCCAGTAAA TGAATAATTACAGTGTGCAGTGATTATTGCAATGATTAATGTATTGATAAGATAATATGAAAACACAGAATTCAAAC AGCAGTGAACTGAGATTAGAATTGTGGAGAGCACTGGCATTTAAGAATGTCACACTTAGAATGTGTCTCTAGGCATT GTTCTGTGCATATATCATCTCAATATTCATTATCTGAAAATTATGAATTAGGTACAAAGCTCAAATAATTTATTTTT TCAGGTTAGCAAGAACTTTTTTTTTTTTTTTTTCTGAGATGGAGCATTGCTATGGTTGCCCAGGCTGGAGTGCAATG GCATGATCCAGGCTCACTGCAACATCTGCCTCCCAGGTTCAAGCGATTCTCCTGCCTCAGCCTCCCAAGTAGCTGGC ATTACAGGCATGTGCCACCACCATGCCTGGCTAATTTTCTATTTTTAGTAGATAGGGGGTTTCACCATGTTGGTCAG GCTGATCTCGAACTCCTAACATCAGGTGATCCACCCTCCTCGGCCTCTGAATGTACTGGGATCACAGGCGTGAGCCA CCACACCCAGCCAAGAATGTGAATTTTGTAGAAGGATATAACCCATATTTCTCTGACCCTAGAGTCCTTAGTATACC TCCCATACCATGTGGCTCATCCTCCTTACATACATTTCCCATCTTTCACCCTACCTTTTCCTTTTTGTTTCAGCTTT TCACTGTGTGTCAAAATCTAGAACCTTATCTCCTACCTGCTCTGAAACCAACAGCAAGTTGACTTCCATTCTAACCC ACATTGGCATTACACTAATTAAAATCGATACTGAGTTCTAAAATCATCTGGGATTTTGGGGACTATGTCTTACTTCA TACTTCCTTGAGATTTCACATTAAATGTTGGTGTTCATTAAAGGTCCTTCATTTAACTTTGTATTCATCACACTCTT GGATTCACAGTTATATCTAAACTCTTATATATAGCCTGTATAATCCCAATTCCCAAGTCTGATTTCTAACCTCTGAC CTCCAACCTCAGTGCCAAACCCATATATCAAACAATGTACTGGGCTTATTTATATAGATGTCCTATAGGCACCTCAG ACTCAGCATGGGTATTTCACTTGTTATACTAAAACTGTTTCTCTTCCAGTGTTTTCCATTTTAGTCATTAGATAGCT ACTTGCCCATTCACCAAGGTCACAGATTAAAATCATTTCCCTACCTCTAATCAACAGTTCAATTCTGCTTCAATTTG TCCCTATCTATTAATCACCACTCTTACTGCCCAGTCAGGTCCTCATTGTTTCCTGAACAAGAGTAGATGCTATTCTT TCCACTTTAAGACCTTATCCTGGCTGGATGCGGTGGCTCAGGCTTGTAAACCCAGCACTTTGGGAGGCCGAGGCAGG CAGATCACTTGAGGTCAGGAGTTCAAGACCAGCCTGACCAACATGGTGAAACCCCATCTCTACTAAAAATACAAAAT CAGCCGGGCGTGTGGTGCATGCCTGCAGTCCCAGCTATTCAGGTGGCTGAGGCAGGAGAATTGCTTGAACCCAGGAG GCGGAGGTTGCGGTGAGCCTAGATTGCACCATTGCACTCTAGCTTGGGCAATAGGGATGAAACTCCATCTCAGAAGA GAAAAGAAAAAAAGACCTTATTCTGTTACACAAATCCTCTCAATGCAATCCATATAGAATAAACATGTAACCAGATC TCCCAATGTGTAAAATCATTTCAGGTAGAACAGAATTAAAGTGAAAAGCCAAGTCTTTGGAATTAACAGACAAAGTT CAAATAACAGTCCTCATGGCCTTAAGAATTTACCTAACATTTTTTTTAGAATCAATTTTCTTATATATGAATTGGAA ACATAATTCCTCCCTCACAAACACATTCTAAGATTTTAAGGAGATATTGATGAAGTACATCATCTGTCATTTTTAAC AGTTAGTGGTAGTGATTCACACAGCACATTATGATCTGTTCTTGTATGTTCTGTTCCATTCTGTATTCTTGACCTGG TTGTATTCTTTCTGAGCTCCAGATCCACATATCTAAGTACATCTTTTTGCATTTTACAAGAGTGCATACAATACAAT GTATCCAAGACTGTATTTCTGATTTTATCGTACCACTAAACTCACAAATGTGGCCCTATTCTTGTGTTCACGACTGA CATCACCGTCATGGTCCAAGTCTGATAATAGAAATGGCATTGTCACTTTCTTCCCTACTGCAACAGAAGCCCAGCTA TTTGTCTCCCATTTTCTCTACTTCTAAAATACATTTCTTCACTAAGTGAGAATAATCTTTTAAAGACACAAATCAAA CCATGCCACCACCTTTCTTGAATTATTCAATATCTTTCGTTGGCTTCCAGGTTACAGAAAAATAACTTGTAACAAAG TTTAAAGGTCATTCATGGCTCCTCTCTACCCTATTTTATAACATTTCCCCTTGTGATCAGAATCTCAGGCACATCAT CCATCTTTCTATATACAAATAAAGTCATATAGTTTGAACTCACCTCTGGTTACTTTTAATCAACCAAATGCTGTAAA ATGCATTTGTATCGCTACGTGTTAAGCAGTAGTTGATTCTTTTCATTTCTTGTTAATATTCTATTCTTTGACTATAC CGTAATTTATCAATTCTACTGTTGGTAAGCATTTAAGTGGCTACCGGTTTGAGGTTTTTATGATTATTGCTGTCATA AGCATTTCTATACATGTCTTTGGATACACACATGCATGTGTTTCTGAATATCTAAAAATGTAATTGCTAGGTAATAG ACTTATCAAGCATCCAGCATTTGTGGATACTATTAAAGGTTTTCCAAAGGGGTTATACTATTGTACAGTGTCACCAA CAGAGTTTGAGTTTCTATTGATCCATATCACCACCAAAATTTGAACTGTCAGTCTTATCTCTTCTCTTGTCTCTTTT TTCCTCTTTTTTTTCCTTCCCTTCCCCTCTCTTCGTTTCTTTTCTCTCCTCTTCTCTTCTTTCCTCTCTTCCCTTCC CTTTCTCTTTCTCTTCCCTATCCCTTCTCCTCTCCTCTCCCCTCCTTTTTTCTCCTCTCCTCTCCATTATTTATTTT TCCTTCTTCTCCTCCATCCCTTCCATCCTCTCTCTTCCCCTCTTCCTTCCTTCCTTTCTCCATTTCTTCCTCCTCTT TCCCTCAATCCTTCCTTTTGGATATGCTCATGGGTGTGTATTTGTCTGCCATTGTGGCATTATTTGAATTCAGAAAA GAGTGAAAAACTACTGGGATCTTCATTCTGGGTCTAATTCCACATTTTTTTTTAAGAACACACTCTGTAAAAATGTT CTGTACTAGCATATTCCCAGGAACTTCGTTAAATTTAATCTGGCTGAATATGGTAAATCTACTTTGCACTTTGCATT CTTTCTTTAGTCATACCATAATTTTAAACATTCAAAATATTTGTATATAATATTTGATTTTATCTGTCATTAAAATG TTAACCTTAAAATTCATGTTTCCAGAACCTATTTCAATAACTGGTAAATAAACACTATTCATTTTTTAAATATTCTT TTAATGGATATTTATTTCAATATAATAAAAAATTAGAGTTTTATTATAGGAAGAATTTACCAAAAGAAGGAGGAAGC AAGCAAGTTTAAACTGCAGCAATAGTTGTCCATTCCAACCTCTCAAAATTCCCTTGGAGACAAAATCTCTAGAGGCA AAGAAGAACTTTATATTGAGTCAACTTGTTAAAACATCTGCTTTTAGATAAGTTTTCTTAGTATAAAGTGACAGAAA CAAATAAGTTAAACTCTAAGATACATTCCACTATATTAGCCTAAAACACTTCTGCAAAAATGAAACTAGGAGGATAT TTTTAGAAACAACTGCTGAAAGAGATGCGGTGGGGAGATATGCAGAGGAGAACAGGGTTTCTGAGTCAAGACACACA TGACAGAACAGCCAATCTCAGGGCAAGTTAAGGGAATAGTGGAATGAAGGTTCATTTTTCATTCTCACAAACTAATG AAACCCTGCTTATCTTAAACCAACCTGCTCACTGGAGCAGGGAGGACAGGACCAGCATAAAAGGCAGGGCAGAGTCG ACTGTTGCTTACACTTTCTTCTGACATAACAGTGTTCACTAGCAACCTCAAACAGACACCATGGTGCATCTGACTCC TGAGGAGAAGACTGCTGTCAATGCCCTGTGGGGCAAAGTGAACGTGGATGCAGTTGGTGGTGAGGCCCTGGGCAGGT TGGTATCAAGGTTATAAGAGAGGCTCAAGGAGGCAAATGGAAACTGGGCATGTGTAGACAGAGAAGACTCTTGGGTT TCTGATAGGCACTGACTCTCTGTCCCTTGGGCTGTTTTCCTACCCTCAGATTACTGGTGGTCTACCCTTGGACCCAG AGGTTCTTTGAGTCCTTTGGGGATCTGTCCTCTCCTGATGCTGTTATGGGCAACCCTAAGGTGAAGGCTCATGGCAA GAAGGTGCTAGGTGCCTTTAGTGATGGCCTGGCTCACCTGGACAACCTCAAGGGCACTTTTTCTCAGCTGAGTGAGC TGCACTGTGACAAGCTGCACGTGGATCCTGAGAACTTCAGGGTGAGTCCAGGAGATGCTTCACTTTTCTCTTTTTAC TTTCTAATCTTACATTTTGGTTCTTTTACCTACCTGCTCTTCTCCCACATTTTTGTCATTTTACTATATTTTATCAT TTAATGCTTCTAAAATTTTGTTAATTTTTTATTTAAATATTCTGCATTTTTTCCTTCCTCACAATCTTGCTATTTTA AATTATTTAATATCCTGTCTTTCTCTCCCAACCCCCTCCCTTCATTTTTCCTTCTCTAACAACAACTCAAATTATGC ATACCAGCTCTCACCTGCTAATTCTGCACTTAGAATAATCCTTTTGTCTCTCCACATGGGTATGGGAGAGGCTCCAA CTCAAAGATGAGAGGCATAGAATACTGTTTTAGAGGCTATAAATCATTTTACAATAAGGAATAATTGGAATTTTATA AATTCTGTAGTAAATGGAATGGAAAGGAAAGTGAATATTTGATTATGAAAGACTAGGCAGTTACACTGGAGGTGGGG CAGAAGTCGTTGCTAGGAGACAGCCCATCATCACACTGATTAATCAATTAATTTGTATCTATTAATCTGTTTATAGT AATTAATTTGTATATGCTATATACACATACAAAATTAAAACTAATTTGGAATTAATTTGTATATAGTATTATACAGC ATATATAGCATATATGTACATATATAGACTACATGCTAGTTAAGTACATAGAGGATGTGTGTGTATAGATATATGTT ATATGTATGCATTCATATATGTACTTATTTATGCTGATGGGAATAACCTGGGGATCAGTTTTGTCTAAGATTTGGGC AGAAAAAAATGGGTGTTGGCTCAGTTTCTCAGAAGCCAGTCTTTATTTCTCTGTTAACCATATGCATGTATCTGCCT ACCTCTTCTCCGCAGCTCTTGGGCAATGTGCTGGTGTGTGTGCTGGCCCGCAACTTTGGCAAGGAATTCACCCCACA AATGCAGGCTGCCTATCAGAAGGTGGTGGCTGGTGTGGCTAATGCCCTGGCTCACAAGTACCATTGAGATCCTGGAC TGTTTCCTGATAACCATAAGAAGACCCTATTTCCCTAGATTCTATTTTCTGAACTTGGGAACACAATGCCTACTTCA AGGGTATGGCTTCTGCCTAATAAAGAATGTTCAGCTCAACTTCCTGATTAATTTCACTTATTTCATTTTTTTGTCCA GGTGTGTAAGAAGGTTCCTGAGGCTCTACAGATAGGGAGCACTTGTTTATTTTACAAAGAGTACATGGGAAAAGAGA AAAGCAAGGGAACCGTACAAGGCATTAATGGGTGACACTTCTACCTCCAAAGAGCAGAAATTATCAAGAACTCTTGA TACAAAGATAATACTGGCACTGCAGAGGTTCTAGGGAAGACCTCAACCCTAAGACATAGCCTCAAGGGTAATAGCTA CGATTAAACTCCAACAATTACTGAGAAAATAATGTGCTCAATTAAAGGCATAATGATTACTCAAGACAATGTTATGT TGTCTTTCTTCCTCCTTCCTTTGCCTGCACATTGTAGCCCATAATACTATACCCCATCAAGTGTTCCTGCTCCAAGA AATAGCTTCCTCCTCTTACTTGCCCCAGAACATCTCTGTAAAGAATTTCCTCTTATCTTCCCATATTTCAGTCAAGA TTCATTGCTCACGTATTACTTGTGACCTCTCTTGACCCCAGCCACAATAAACTTCTCTATACTACCCAAAAAATCTT TCCAAACCCTCCCCGACACCATATTTTTATATTTTTCTTATTTATTTCATGCACACACACACACTCCGTGCTTTATA AGCAATTCTGCCTATTCTCTACCTTCTTACAATGCCTACTGTGCCTCATATTAAATTCATCAATGGGCAGAAAGAAA ATATTTATTCAAGAAAACAGTGAATGAATGAACGAATGAGTAAATGAGTAAATGAAGGAATGATTATTCCTTGCTTT AGAACTTCTGGAATTAGAGGACAATATTAATAATACCATCGCACAGTGTTTCTTTGTTGTTAATGCTACAACATACA AAGAGGAAGCATGCAGTAAACAACCGAACAGTTATTTCCTTTCTGATCATAGGAGTAATATTTTTTTCCTTGAGCAC ATTTTTGCCATAGGTAAAATTAGAAGGATTTTTAGAACTTTCTCAGTTGTATACATTTTTAAAAATCTGTATTATAT GCATGTTGATTAATTTTAAACTTACTTGAATACCTAAACAGAATCTGTTGTTTCCTTGTGTTTGAAAGTGCTTTCAC AGTAACTCTGTCTGTACTGCCAGAATATACTGACAATGTGTTATAGTTAACTGTTTTGATCACAACATTTTGAATTG ACTGGCAGCAGAAGCTCTTTTTATATCCATGTGTTTTCCTTAAGTCATTATACATAGTAGGCATGAGACTCTTTATA CTGAATAAGATATTTAGGAACCACTGGTTTACATATCAGAAGCAGAGCTACTCAGGGCATTTTGGGGAAGATCACTT TCACATTCCTGAGCATAGGGAAGTTCTCATAAGAGTAAGATATTAAAAGGAGATACTTGTGTGGTATTCGAAAGACA GTAAGAGAGATTGTAGACCTTATGATCTTGATAGGGAAAACAAACTACATTCCTTTCTCCAAAAGTCAAAAAAAAAG AGCAAATATAGCTTACTATACCTTCTATTCCTACACCATTAGAAGTAGTCAGTGAGTCTAGGCAAGATGTTGGCCCT AAAAATCCAAATACCAGAGAATTCATGAGAACATCACCTGGATGGGACATGTGCCGAGCAACACAATTACTATATGC TAGGCATTGCTATCTTCATATTGAAGATGAGGAGGTCAAGAGATGAAAAAAGACTTGGCACCTTGTTGTTATATTAA AATTATTTGTTAGAGTAGAGCTTTTGTAAGAGTCTAGGAGTGTGGGAGCTAAATGATGATACACATGGACACAAAGA ATAGATCAACAGACACCCAGGCCTACTTGAGGGTTGAGGGTGGGAAGAGGGAGACGATGAAAAAGAACCTATTGGGT ATTAAGTTCATCACTGAGTGATGAAATAATCTGTACATCAAGACCCAGTGATATGCAATTTACCTATATAACTTGTA CATGTACCCCCAAATTTAAAATAAAGTTAAAACAAAGTATAGGAATGGAATTAATTCCTCAAGATTTGGCTTTAATT TTATTTGATAATTTATCAAATGGTTGTTTTTCTTTTCTCACTATGGCGTTGCTTTATAAACTATGTTCAGTATGTCT GAATGAAAGGGTGTGTGTGTGTGTGAAAGAGAGGGAGAGAGGAAGGGAAGAGAGGACGTAATAATGTGAATTTGAGT TCATGAAAATTTTTCAATAAAATAATTTAATGTCAGGAGAATTAAGCCTAATAGTCTCCTAAATCATCCATCTCTTG AGCTTCAGAGCAGTCCTCTGAATTAATGCCTACATGTTTGTAAAGGGTGTTCAGACTGAAGCCAAGATTCTACCTCT AAAGAGATGCAATCTCAAATTTATCTGAAGACTGTACCTCTGCTCTCCATAAATTGACACCATGGCCCACTTAATGA GGTTAAAAAAAAGCTAATTCTGAATGAAAATCTGAGCCCAGTGGAGGAAATATTAATGAACAAGGTGCAGACTGAAA TATAAATTTTCTGTAATAATTATGCATATACTTTAGCAAAGTTCTGTCTATGTTGACTTTATTGCTTTTGGTAAGAA ATACAACTTTTTAAAGTGAACTAAACTATCCTATTTCCAAACTATTTTGTGTGTGTGCGGTTTGTTTCTATGGGTTC TGGTTTTCTTGGAGCATTTTTATTTCATTTTAATTAATTAATTCTGAGAGCTGCTGAGTTGTGTTTACTGAGAGATT GTGTATCTGCGAGAGAAGTCTGTAGCAAGTAGCTAGACTGTGCTTGACCTAGGAACATATACAGTAGATTGCTAAAA TGTCTCACTTGGGGAATTTTAGACTAAACAGTAGAGCATGTATAAAAATACTCTAGTCAAGTGCTGCTTTTGAAACA AATGATAAAACCACACTCCCATAGATGAGTGTCATGATTTTCATGGAGGAAGTTAATATTCATCCTCTAAGTATACC CAGACTAGGGCCATTCTGATATAAAACATTAGGACTTAAGAAAGATTAATAGACTGGAGTAAAGGAAATGGACCTCT GTCTCTCTCGCTGTCTCTTTTTTGAGGACTTGTGTGTGTGTGTGTGTGTGTGTGTGTGTGTGTTGTGGTCAGTGGGG CTGGAATAAAAGTAGAATAGACCTGCACCTGCTGTGGCATCCATTCACAGAGTAGAAGCAAGCTCACAATAGTGAAG ATGTCAGTAAGCTTGAATAGTTTTTCAGGAACTTTGAATGCTGATTTAGATTTGAAACTGAGGCTCTGACCATAACC AAATTTGCACTATTTATTGCTTCTTGAAACTTATTTGCCTGGTATGCCTGGGCTTTTGATGGTCTTAGTATAGCTTG CAGCCTTGTCCCTGCAGGGTATTATGGGTAATAGAAAGAAAAGTCTGCGTTACACTCTAGTCACACTAAGTAACTAC CATTGGAAAAGCAACCCCTGCCTTGAAGCCAGGATGATGGTATCTGCAGCAGTTGCCAACACAAGAGAAGGATCCAT AGTTCATCATTTAAAAAAGAAAAAAAATAGAAAAAGGAAAACTATTTCTGAGCATAAGAAGTTGTAGGGTAAGTCT TTAAGAAGGTGACAATTTCTGCCAATCAGGATTTCAAAGCTCTTGCTTTGACAATTTTGGTCTTTCAGAATACTATA AATATAACCTATATTATAATTTCATAAAGTCTGTGCATTTTCTTTGACCCAGGATATTTGCAAAAGACATATTCAAA CTTCCGCAGAACACTTTATTTCACATATACATGCCTCTTATATCAGGGATGTGAAACAGGGTCTTGAAAACTGTCTA AATCTAAAACAATGCTAATGCAGGTTTAAATTTAATAAAATAAAATCCAAAATCTAACAGCCAAGTCAAATCTGTAT GTTTTAACATTTAAAATATTTTAAAGACGTCTTTTCCCAGGATTCAACATGTGAAATCTTTTCTCAGGGATACACGT GTGCCTAGATCCTCATTGCTTTAGTTTTTTACAGAGGAATGAATATAAAAAGAAAATACTTAAATTTTATCCCTCTT ACCTCTATAATCATACATAGGCATAATTTTTTAACCTAGGCTCCAGATAGCCATAGAAGAACCAAACACTTTCTGCG TGTGTGAGAATAATCAGAGTGAGATTTTTTCACAAGTACCTGATGAGGGTTGAGACAGGTAGAAAAAGTGAGAGATC TCTATTTATTTAGCAATAATAGAGAAAGCATTTAAGAGAATAAAGCAATGGAAATAAGAAATTTGTAAATTTCCTTC TGATAACTAGAAATAGAGGATCCAGTTTCTTTTGGTTAACCTAAATTTTATTTCATTTTATTGTTTTATTTTATTTT ATTTTATTTTATTTTGTGTAATCGTAGTTTCAGAGTGTTAGAGCTGAAAGGAAGAAGTAGGAGAAACATGCAAAGTA AAAGTATAACACTTTCCTTACTAAACCGACTGGGTTTCCAGGTAGGGGCAGGATTCAGGATGACTGACAGGGCCCTT AGGGAACACTGAGACCCTACGCTGACCTCATAAATGCTTGCTACCTTTGCTGTTTTAATTACATCTTTTAATAGCAG GAAGCAGAACTCTGCACTTCAAAAGTTTTTCCTCACCTGAGGAGTTAATTTAGTACAAGGGGAAAAAGTACAGGGGG ATGGGAGAAAGGCGATCACGTTGGGAAGCTATAGAGAAAGAAGAGTAAATTTTAGTAAAGGAGGTTTAAACAAACAA AATATAAAGAGAAATAGGAACTTGAATCAAGGAAATGATTTTAAAACGCAGTATTCTTAGTGGACTAGAGGAAAAAA ATAATCTGAGCCAAGTAGAAGACCTTTTCCCCTCCTACCCCTACTTTCTAAGTCACAGAGGCTTTTTGTTCCCCCAG ACACTCTTGCAGATTAGTCCAGGCAGAAACAGTTAGATGTCCCCAGTTAACCTCCTATTTGACACCACTGATTACCC CATTGATAGTCACACTTTGGGTTGTAAGTGACTTTTTATTTATTTGTATTTTTGACTGCATTAAGAGGTCTCTAGTT TTTTATCTCTTGTTTCCCAAAACCTAATAAGTAACTAATGCACAGAGCACATTGATTTGTATTTATTCTATTTTTAG ACATAATTTATTAGCATGCATGAGCAAATTAAGAAAAACAACAACAAATGAATGCATATATATGTATATGTATGTGT GTATATATACACATATATATATATATTTTTTTTCTTTTCTTACCAGAAGGTTTTAATCCAAATAAGGAGAAGATATG CTTAGAACTGAGGTAGAGTTTTCATCCATTCTGTCCTGTAAGTATTTTGCATATTCTGGAGACGCAGGAAGAGATCC ATCTACATATCCCAAAGCTGAATTATGGTAGACAAAGCTCTTCCACTTTTAGTGCATCAATTTCTTATTTGTGTAAT AAGAAAATTGGGAAAACGATCTTCAATATGCTTACCAAGCTGTGATTCCAAATATTACGTAAATACACTTGCAAAGG AGGATGTTTTTAGTAGCAATTTGTACTGATGGTATGGGGCCAAGAGATATATCTTAGAGGGAGGGCTGAGGGTTTGA AGTCCAACTCCTAAGCCAGTGCCAGAAGAGCCAAGGACAGGTACGGCTGTCATCACTTAGACCTCACCCTGTGGAGC CACACCCTAGGGTTGGCCAATCTACTCCCAGGAGCAGGGAGGGCAGGAGCCAGGGCTGGGCATAAAAGTCAGGGCAG AGCCATCTATTGCTTACATTTGCTTCTGACACAACTGTGTTCACTAGCAACCTCAAACAGACACCATGGTGCACCTG ACTCCTGAGGAGAAGTCTGCCGTTACTGCCCTGTGGGGCAAGGTGAACGTGGATGAAGTTGGTGGTGAGGCCCTGGG CAGGTTGGTATCAAGGTTACAAGACAGGTTTAAGGAGACCAATAGAAACTGGGCATGTGGAGACAGAGAAGACTCTT GGGTTTCTGATAGGCACTGACTCTCTCTGCCTATTGGTCTATTTTCCCACCCTTAGGCTGCTGGTGGTCTACCCTTG GACCCAGAGGTTCTTTGAGTCCTTTGGGGATCTGTCCACTCCTGATGCTGTTATGGGCAACCCTAAGGTGAAGGCTC ATGGCAAGAAAGTGCTCGGTGCCTTTAGTGATGGCCTGGCTCACCTGGACAACCTCAAGGGCACCTTTGCCACACTG AGTGAGCTGCACTGTGACAAGCTGCACGTGGATCCTGAGAACTTCAGGGTGAGTCTATGGGACCCTTGATGTTTTCT TTCCCCTTCTTTTCTATGGTTAAGTTCATGTCATAGGAAGGGGAGAAGTAACAGGGTACAGTTTAGAATGGGAAACA GACGAATGATTGCATCAGTGTGGAAGTCTCAGGATCGTTTTAGTTTCTTTTATTTGCTGTTCATAACAATTGTTTTC TTTTGTTTAATTCTTGCTTTCTTTTTTTTTCTTCTCCGCAATTTTTACTATTATACTTAATGCCTTAACATTGTGTA TAACAAAAGGAAATATCTCTGAGATACATTAAGTAACTTAAAAAAAAACTTTACACAGTCTGCCTAGTACATTACTA TTTGGAATATATGTGTGCTTATTTGCATATTCATAATCTCCCTACTTTATTTTCTTTTATTTTTAATTGATACATAA TCATTATACATATTTATGGGTTAAAGTGTAATGTTTTAATATGTGTACACATATTGACCAAATCAGGGTAATTTTGC ATTTGTAATTTTAAAAAATGCTTTCTTCTTTTAATATACTTTTTTGTTTATCTTATTTCTAATACTTTCCCTAATCT CTTTCTTTCAGGGCAATAATGATACAATGTATCATGCCTCTTTGCACCATTCTAAAGAATAACAGTGATAATTTCTG GGTTAAGGCAATAGCAATATTTCTGCATATAAATATTTCTGCATATAAATTGTAACTGATGTAAGAGGTTTCATATT GCTAATAGCAGCTACAATCCAGCTACCATTCTGCTTTTATTTTATGGTTGGGATAAGGCTGGATTATTCTGAGTCCA AGCTAGGCCCTTTTGCTAATCATGTTCATACCTCTTATCTTCCTCCCACAGCTCCTGGGCAACGTGCTGGTCTGTGT GCTGGCCCATCACTTTGGCAAAGAATTCACCCCACCAGTGCAGGCTGCCTATCAGAAAGTGGTGGCTGGTGTGGCTA ATGCCCTGGCCCACAAGTATCACTAAGCTCGCTTTCTTGCTGTCCAATTTCTATTAAAGGTTCCTTTGTTCCCTAAG TCCAACTACTAAACTGGGGGATATTATGAAGGGCCTTGAGCATCTGGATTCTGCCTAATAAAAAACATTTATTTTCA TTGCAATGATGTATTTAAATTATTTCTGAATATTTTACTAAAAAGGGAATGTGGGAGGTCAGTGCATTTAAAACATA AAGAAATGAAGAGCTAGTTCAAACCTTGGGAAAATACACTATATCTTAAACTCCATGAAAGAAGGTGAGGCTGCAAA CAGCTAATGCACATTGGCAACAGCCCTGATGCCTATGCCTTATTCATCCCTCAGAAAAGGATTCAAGTAGAGGCTTG ATTTGGAGGTTAAAGTTTTGCTATGCTGTATTTTACATTACTTATTGTTTTAGCTGTCCTCATGAATGTCTTTTCAC TACCCATTTGCTTATCCTGCATCTCTCAGCCTTGACTCCACTCAGTTCTCTTGCTTAGAGATACCACCTTTCCCCTG AAGTGTTCCTTCCATGTTTTACGGCGAGATGGTTTCTCCTCGCCTGGCCACTCAGCCTTAGTTGTCTCTGTTGTCTT ATAGAGGTCTACTTGAAGAAGGAAAAACAGGGGGCATGGTTTGACTGTCCTGTGAGCCCTTCTTCCCTGCCTCCCCC ACTCACAGTGACCCGGAATCTGCAGTGCTAGTCTCCCGGAACTATCACTCTTTCACAGTCTGCTTTGGAAGGACTGG GCTTAGTATGAAAAGTTAGGACTGAGAAGAATTTGAAAGGGGGCTTTTTGTAGCTTGATATTCACTACTGTCTTATT ACCCTATCATAGGCCCACCCCAAATGGAAGTCCCATTCTTCCTCAGGATGTTTAAGATTAGCATTCAGGAAGAGATC AGAGGTCTGCTGGCTCCCTTATCATGTCCCTTATGGTGCTTCTGGCTCTGCAGTTATTAGCATAGTGTTACCATCAA CCACCTTAACTTCATTTTTCTTATTCAATACCTAGGTAGGTAGATGCTAGATTCTGGAAATAAAATATGAGTCTCAA GTGGTCCTTGTCCTCTCTCCCAGTCAAATTCTGAATCTAGTTGGCAAGATTCTGAAATCAAGGCATATAATCAGTAA TAAGTGATGATAGAAGGGTATATAGAAGAATTTTATTATATGAGAGGGTGAAACCTAAAATGAAATGAAATCAGACC CTTGTCTTACACCATAAACAAAAATAAATTTGAATGGGTTAAAGAATTAAACTAAGACCTAAAACCATAAAAATTTT TAAAGAAATCAAAAGAAGAAAATTCTAATATTCATGTTGCAGCCGTTTTTTGAATTTGATATGAGAAGCAAAGGCAA CAAAAGGAAAAATAAAGAAGTGAGGCTACATCAAACTAAAAAATTTCCACACAAAAAAGAAAACAATGAACAAATGA AAGGTGAACCATGAAATGGCATATTTGCAAACCAAATATTTCTTAAATATTTTGGTTAATATCCAAAATATATAAGA AACACAGATGATTCAATAACAAACAAAAAATTAAAAATAGGAAAATAAAAAAATTAAAAAGAAGAAAATCCTGCCAT TTATGCGAGAATTGATGAACCTGGAGGATGTAAAACTAAGAAAAATAAGCCTGACACAAAAAGACAAATACTACACA ACCTTGCTCATATGTGAAACATAAAAAAGTCACTCTCATGGAAACAGACAGTAGAGGTATGGTTTCCAGGGGTTGGG GGTGGGAGAATCAGGAAACTATTACTCAAAGGGTATAAAATTTCAGTTATGTGGGATGAATAAATTCTAGATATCTA ATGTACAGCATCGTGACTGTAGTTAATTGTACTGTAAGTATATTTAAAATTTGCAAAGAGAGTAGATTTTTTTGTTT TTTTAGATGGAGTTTTGCTCTTGTTGTCCAGGCTGGAGTGCAATGGCAAGATCTTGGCTCACTGCAACCTCCGCCTC CTGGGTTCAAGCAAATCTCCTGCCTCAGCCTCCCGAGTAGCTGGGATTACAGGCATGCGACACCATGCCCAGCTAAT TTTGTATTTTTAGTAGAGACGGGGTTTCTCCATGTTGGTCAGGCTGATCCGCCTCCTCGGCCACCAAAGGGCTGGGA TTACAGGCGTGACCACCGGGCCTGGCCGAGAGTAGATCTTAAAAGCATTTACCACAAGAAAAAGGTAACTATGTGAG ATAATGGGTATGTTAATTAGCTTGATTGTGGTAATCATTTCACAAGGTATACATATATTAAAACATCATGTTGTACA CCTTAAATATATACAATTTTTATTTGTGAATGATACCTCAATAAAGTTGAAGAATAATAAAAAAGAATAGACATCAC ATGAATTAAAAAACTAAAAAATAAAAAAATGCATCTTGATGATTAGAATTGCATTCTTGATTTTTCAGATACAAATA TCCATTTGACTGTTTACTCTTTTCCAAAACAATACAATAAATTTTAGCACTTTATCTTCATTTTCCCCTTCCCAATC TATAATTTTATATATATATATTTTAGATATTTTGTATAGTTTTACTCCCTAGATTTTCTAGTGTTATTATTAAATAG TGAAGAAATGTTTACACTTATGTACAAAATGTTTTGCATGCTTTTCTTCATTTCTAACATTCTCTCTAAGTTTATTC TATTTTTTCCTGATTATCCTTAATATTATCTCTTTCTGCTGGAAATATATTGTTACTTTTGGTTTATCTAAAAATGG CTTCATTTTCTTCATTCTAAAATCATGTTAAATTAATACCACTCATGTGTAAGTAAGATAGTGGAATAAATAGAAAT CCAAAAACTAAATCTCACAAAATATAATAATGTGATATATAAAAATATAGCTTTTAAATTTAGCTTGGAAATAAAAA ACAAACAGTAATTGAACAACTATACTTTTTGAAAAGAGTAAAGTGAAATGCTTAACTGCATATACCACAATCGATTA CACAATTAGGTGTGAAGGTAAAATTCAGTCACGAAAAAACTAGAATAAAAATATGGGAAGACATGTATATAATCTTA GAGATAACAGTGTTATTTAATTATCAACCCAAAGTAGAAACTATCAAGGGAGAAATAAATTCAGTCAACAATAAAAG CATTTAAGAAGTTATTCTAGGCTGGGAGCGGTGGCTCACACCTGCAATTGCAGCACTTTGGGAGGCCTAGACAGGCG GATCACGACGTCAGGAGTTCAAGATCAGCCTGGCCAACATAGTGAAACCTCATCGCTACTAAAAATATAAAAACTTA GCCTGGCGTGGTGGCAGGCATGTGTAATCCCAGCAATTTGGGAGGCTGAGGCAGGAGAATCGCTTGATCCTGGGAGG CAGAGGTTGCAGTGAGCCAAGATTGTGCCACTGCATTCCAGCCCAGGTGACAGCATGAGACTCCGTCACAAAAAAAA AAGAAAAAAAAGGGGGGGGGGGGCGGTGGAGCCAAGATGACCGAATAGGAACAGCTCCAGTCTATAGCTCCCATCGT GAGTGACGCAGAAGACGGGTGATTTCTGCATTTCCAACTGAGGTACCAGGTTCATCTCACAGGGAAGTGCCAGGCAG TGGGTGCAGGACAGTAGTGCAGTGCACTGTGCATGAGCCGAAGCAGGGCGAGGCATCACCTCACCCGGGAAGCACAA GGGGTCAGGGAATTCCCTTTCCTAGTCAAAGAAAAGGGTGACAGATGGCACCTGGAAAATCGGGTCACTCCCGCCCT AATACTGCGCTCTTCCAACAAGCTTAACAAATGGCACACCAGGAGATTATATCCCATGCCTGGCTCAGAGGGTCCTA CGCCCATGGAGCCTCGCTCATTGCTAGCACAGCAGTCTGAGGTCAAACTGCAAGGTGGCAGTGAGGCTGGGGGAGGG GTGCCCACCATTGTCCAGGCTTGAGCAGGTAAACAAAGCCGCCTGGAAGCTCGAACTGGGTGGAGCCCACCACAGCT CAAGGAGGCCTGCCTGCCTCTGTAGGCTCCACCTCTAGGGGCAGGGCACAGACAAACAAAAGACAACAAGAACCTCT GCAGACTTAAATGTCCCTGTCTGACAGCTTTGAAGAGAGTAGTGGTTCTCCCAGCACATAGCTTCAGATCTGAGAAC AGGCAGACTGCCTCCTCAAGTGGGTCCCTGACCCCCGAGTAGCCTAACTGGGAGGCATCCCCCAGTAGGGCGGACTG ACACCTCACATGGCTGGTACTCCTCTAAGACAAAACTTCCAGAGGAATGATCAGGCAGCAGCATTTGCGGTTCACCA ATATCCACTGTTCTGCAGCCACCGCTGCTGATACCCAGGAAAACAGCATCTGGAGTGGACCTCCAGTAAACTCCAAC AGACCTGCAGCTGAGGGTCCTGACTGTTAGAAGGAAAACTAACAAACAGAAAGGACATCCACACCAAAAACCCATCT GTACATCACCATCATCAAAGACCAAAGGTAGATAAAACCATAAAGATGGGGAAAAAGCAGAGCAGAAAAACTGGACA CTCTAAAAATGAGAGTGCCTCTCCTTCTCCAAAGTAACGCAGCTCCTCACCAGCAATGGAACAAAGCTGGGCAGAGA ATGACTTTGACGAGTTGAGAGAGGAAGGCTTCAGAAGATCAAACTACTCCAAGCTAAAGGAGGAAGTTCGAACAAAC GGCAAAGAAGTAAAAAACTTTGAAAAAAAATTAGATGAATGGATAACTAGAATAACCAATGCACAGAAGTCCTTAAA GGACCTGATGGAGCTGAAAACCAAGGCAGGAGAACTACGTGACAAATACACAAGCCTCAGTAACCGATGAGATCAAC TGGAAGAAAGGGTATCAATGACGGAAGATGAAATGAATGAAATGAAGCATGAAGAGAAGTTTAGAGAAAAAAGAATA AAAAGAAACGAACAAAGCCTCCAAGAAATATGGGACTATGTGAAAAGACCAAATCTACATCTAATTGGTGTAGCTGA AAGTGATGGGGAGAATGGAACCAAGTTGGAAAACACTCTGCAGGATATTATCCAGGAGAACTTCCCCAATCTAGCAA GGCAGCCCAAATTCACATTCAGGAAATACAGAGAACGCCACAAAGATACTCCTAGAGAAAAGCAACTCCAAGACACA TAACTGACAGATTCACCAAAGTTGAAATGAAGGAAAAAATGTTAAGGGCAGCCAGAGAGAAAGGTCGGGTTACCCAC AAAGGGAAGCCCATCAGACTAACAGCTGATCTATCGGCAGAAACTCTACAAGCCAGAAGAAAGTGGGGGCCAATATT CAACATTGTTAAAGAAAAGAATTTTCGGCCCAGAATTTCATATCCAGCCAAACTAAGCTTCATAAGCATTGGAGAAA TAAAATCCTTTACAGACAAGCAAATGCTGAGAGATTTTGTCACCACCAGGCCTGCCCTACAAGAGCTCCTGAAGGAA GCACTAAACATGGAAAGGAACAACTAGTATCAGCCACTGCAAAAACATGCCAAATTGTAAACGACCATCAAGGCTAG GAAGAAACTGCATCAAGGAGCAAAATAACCAGCTAACATCATAATGACAGGATCAAATTCATACATAACAATACTCA CCTTAAATGTAAATAGGCTAAATGCTCCAATTAAAAGACACAGACTGGCAAATTGGATAAGGAGTCAAGACCCATCT GTCGTTATGTATTCAGGAAACCCATCTCACGTGCAGAGACACACATAGGCTCGAAATAAAAGGATGGAGGAATATCT ACCAAGCAAATGGAAAACAAAAAAAGGCAGGGGTTGCAATCCTAGTCTCTGATAAAACAGATTTTAAACCAACAAAG ATCAAAAGAGACAAAGAAGGCCATTACATAATGGCAAAGGGATCTATTCAAGAAGAAGAACTAACTATACTAAATAT ATATGCACCCAATACAGGAGCACCCAGATTCATAAAACAAGTCCTGAGTGACCTACAAAGAGACTTAGATGCCCACA CAATAATAATGGGAGACTTTAACACCCCACTGTCAACATTAGACAGATCAACGAGACAGAAAGTTAACAAGGATATC CAGGAATTGGACTCAGCTCTGCACCAAGCAGACCTAATAGACATCTACAGAACTCTCCACCCCAAATCAACAGAATA TACATTCTTTTCAGCACCACACCACACCTATTCCAAAACTGACCACATAGTTGGAAGTAAAGCTCTCCTCAGCAAAT GTAAAAGAACAGAAACTATAACAAACTGTCTCTCAGACCACAGTGCAATCAAACTAGAACTCAGGATTAAGAAACTC ACTCAAAACCACTCAGCTACATGGAAACTGAACAGCCTGCTCCTGAATGACTACTGGGTACATAACAAAATGAAGGC AGAAATAAAGATGTTCTTTGAAACAACGAGAACAAAGACACAACACACCAGAATCTCTGAGACACATTCAAAGCAGT GTGTAGAGGGAAATTTATAGCACTAAATGCCCACAAGGGAAAGCAGGAAAGATCTAAAATTGACACCCTAACATCAC AATTAAAAAACTAGAGAAGCAGGAGCAAACACATTCAAAAGCTAACAGAAGACAAGAAATAACTAAGATCAGAGCAG AAGTGAAGAAGATAGAGACACAAAAAACCCTTCAAAAAAATCAATGAATCCAGAAGCTGTTTTTTTGAAAAGATCAA CAAAATTGATAGACTGCTAGCAAGACTAATAAAGAAGAAAGGGGAGAAGAATCAAATAGACGCAATAAAAAATGACA CGGGGTATCACCACTGATCCCACAGAAATACAAACTACCGTCAGAGAATACTATAAACACCTCTACGCAAATAAACT AGAAAATCTAGAAGAAATGGATAAATTCCTCGACACATACACTCTGCCAAGACTAAACCAGGAAGAAGTTGTATCTC TGAATAGACCAATAACAGGCTCTGAAATTGAGGCAATAATTAATAGCTTATCAACCAAAAAAAGTCCGGGACCAGTA GGATTCATAGCCGAATTCTACCAGAGGTACAAGGAGGAGCTGGTACCATTCCTTCTGAAACTATTCCAATCAATAGA AAAAGAGGGAATCCTCCCTAACTCATTTTATGAGGCCAGCATCATCCTGATACCAAAGCCTGACAGAGACACAACAA AAAAAGAGAATGTTACACCAATATCCTTGATGAACATCGATGCAAAAATCCTCAATAAAATACTGGCAAACTGAATC CAGCAGCACATCAAAAAGCTTATCCTCCATGATCAAGTGGGCTTCATCCCTGCCATGCAAGGCTGGTTCAACATACG AAATCAATAAACATAATCCAGCATATAAACAGAACCAAAGACACAAACCATATGATTATCTCAATAGATGCAGAAAA GGCCTTTGACAAAATTCAACAATGCTTCATGCTAAAAACTCTCAATAAATTAGGTATTGATGGGACATATCTCAAAA TAATAAGAGCTATCTATGACAAACCCACAGCCAATATCATACTGAGTGGACAAAAACTGGAAGCATTCCCTTTGAAA ACTGGCACAAGGCAGGGATGCCCTCTCTCACCACTCCTATTCAACATAGTGTTGGAAGTTCTGGCCAGGGCAATCAG GCAGGAGAAGGAAATAAAGGGCATTCAATTAGGAAAAGAGGAAGGTGAAATTGTCCCTGTTTGCAGATGACATGATT GTATATCTAGAAAACCCCATTGTCTCAGCCCAAAATCTCCTTAAGCTGATAAGCAACTTCAGCAAAGTCTCAGGATA TAAAATCAGTGTGCAAAAATCACAAGTATTCCTATGCACCAATAACAGACAAACAGAGAGCCAAATCATGAGTGAAC TCCCATTCACAATTGCTTCAAAGAGAATAAAATACCTAGGAATCCAACTTACAAGGGATGTGAAGGACCTCTTCAAG GAGAACTACAAACCACTGCTCAATGAAATAAAAGAGGATACAAACAAATGGAAGAACATTCCATGCTTATGGGTAGG AAGAATCATATCGTGAAAATGGTCATACTGCCCAAGGTAATTTATAGATTCAATGCCATCCCCATCAAGCTACCAAT GACTTTCTTCACAGAACTGGAAAAAACTACTTTAAAGTTCATATGGAATCAAAAAAGAGCCCACATCACCAAGGCAA TCCTAAGCCAAAAGAACAAAGCTGGAGGCATCACGCTACCTGACTTCAAACTATACTACAATGCTACGGTAACCAAA ACAGCATGGTACTGGTACCAAAACAGAGATCTAGACCAATGGAACAGAACAGAGCCCTCAGAAATAATGCCGCATAT CTACAACTATCCGATCTTTGACAAACCTGAGAGAAACAAGCAATGGGGAAAGGATTCCCTATTTAATAAATGGTGCT GGGAAAACTGGCTAGCCATATGTAGAAAGCTGAAACTGGATCCTTCCTTACACCTTATACAAAAATTAATTCAAGAT GGATTAAAGACTTAAACATTAGACCTAAAACCATAAAAACCCTAGAAAAAAACCTAGGCAATACCATTCAGGACATA GGCATGGGCAAGGACTTCATGTCTAAAACACCAAAACGAATGGCAACAAAAGACAAAATGGACAAACGGGATCTAAT TAAACTAAAGAGCTTCTGCACAGCTAAAGAAACTACCATCAGAGTGAACAGGCAACCTACAAAATGGGAGAAAATTT TTGCAATCTACTCATCTGACAAAGGGCTAATATCCAGAATCTACAATGAACTCAAACAAATTTACAAGAAAAAACAA ACAACCCCATCAAAAAGTGGGCAAAGGATATGAACAGACACTTCTCAAAAGAAGACATTTATGTAATCAAAAAACAC ATGAAAAAATGCTCATCATCACTAGCCATCAGAGAAATGCAAATCAAAACCACAATGAGATACCATCTCACACCAGT TAGAATGGCGATCATTAAAAAGTCAGGAAACAACAGGTGCTGGAGAGGATGTGGAGAAACAGGAACAACTTTTACAC TGTTGGTGGGACTGTAAACTAGTTCAACCATTGCGGAAGTCAGTGTGGCAATTCCTCAGGAATCTAGAACTAGAAAT ACCATTTGACCCAGCCATCCCATTACTGGGTAGATACCCAAAGGATTATAAATCATGCTGCTATAAAGACACATGCA CACGTATGTTTATTGCAGCACTATTCACAATAGCAAAGACTTGGAACCAACCCAAATGTCCAACAACGATAGATTGG ATTAAGAAAATGTGGCACATATACACCATGGAATACTATGCAGCCATAAAAAATGATGAGTTCATGTCCTTTGTAGG GACATGGATGAAGCTGGAAACTATCATTCTCAGCAAACTATCACAAGGACAATAAACCAAACACCGCATGTTCTCAC TCATAGGTGGGAATTGAACAATGAGAACACATGGACACATGAAGAGGAACATCACACTCTGGGGACTGTTATGGGGT GGGGGGCAGGGGCAGGGATAGCACTAGGAGATATACCTAATGCTAAATGACGAGTTAATGGGTGCAGCACACCAACA TGGCACATGTATACATATATAACAAACCTGCCGTTGTGCACATGTACCCTAAAACTTGAAGTATAATAATAAAAAAA AGTTATCCTATTAAAACTGATCTCACACATCCGTAGAGCCATTATCAAGTCTTTCTCTTTGAAACAGACAGAAATTT AGTGTTTTCTCAGTCAGTTAAC

GenBank Accession No. U01317.1 GAATTCTAATCTCCCTCTCAACCCTACAGTCACCCATTTGGTATATTAAAGATGTGTTGTCTACTGTCTAGTATCCC TCAAGTAGTGTCAGGAATTAGTCATTTAAATAGTCTGCAAGCCAGGAGTGGTGGCTCATGTCTGTAATTCCAGCACT GGAGAGGTAGAAGTGGGAGGACTGCTTGAGCTCAAGAGTTTGATATTATCCTGGACAACATAGCAAGACCTCGTCTC TACTTAAAAAAAAAAAAATTAGCCAGGCATGTGATGTACACCTGTAGTCCCAGCTACTCAGGAGGCCGAAATGGGAG GATCCCTTGAGCTCAGGAGGTCAAGGCTGCAGTGAGACATGATCTTGCCACTGCACTCCAGCCTGGACAGCAGAGTG AAACCTTGCCTCACGAAACAGAATACAAAAACAAACAAACAAAAAACTGCTCCGCAATGCGCTTCCTTGATGCTCTA CCACATAGGTCTGGGTACTTTGTACACATTATCTCATTGCTGTTCGTAATTGTTAGATTAATTTTGTAATATTGATA TTATTCCTAGAAAGCTGAGGCCTCAAGATGATAACTTTTATTTTCTGGACTTGTAATAGCTTTCTCTTGTATTCACC ATGTTGTAACTTTCTTAGAGTAGTAACAATATAAAGTTATTGTGAGTTTTTGCAAACACAGCAAACACAACGACCCA TATAGACATTGATGTGAAATTGTCTATTGTCAATTTATGGGAAAACAAGTATGTACTTTTTCTACTAAGCCATTGAA ACAGGAATAACAGAACAAGATTGAAAGAATACATTTTCCGAAATTACTTGAGTATTATACAAAGACAAGCACGTGGA CCTGGGAGGAGGGTTATTGTCCATGACTGGTGTGTGGAGACAAATGCAGGTTTATAATAGATGGGATGGCATCTAGC GCAATGACTTTGCCATCACTTTTAGAGAGCTCTTGGGGACCCCAGTACACAAGAGGGGACGCAGGGTATATGTAGAC ATCTCATTCTTTTTCTTAGTGTGAGAATAAGAATAGCCATGACCTGAGTTTATAGACAATGAGCCCTTTTCTCTCTC CCACTCAGCAGCTATGAGATGGCTTGCCCTGCCTCTCTACTAGGCTGACTCACTCCAAGGCCCAGCAATGGGCAGGG CTCTGTCAGGGCTTTGATAGCACTATCTGCAGAGCCAGGGCCGAGAAGGGGTGGACTCCAGAGACTCTCCCTCCCAT TCCCGAGCAGGGTTTGCTTATTTATGCATTTAAATGATATATTTATTTTAAAAGAAATAACAGGAGACTGCCCAGCC CTGGCTGTGACATGGAAACTATGTAGAATATTTTGGGTTCCATTTTTTTTTCCTTCTTTCAGTTAGAGGAAAAGGGG CTCACTGCACATACACTAGACAGAAAGTCAGGAGCTTTGAATCCAAGCCTGATCATTTCCATGTCATACTGAGAAAG TCCCCACCCTTCTCTGAGCCTCAGTTTCTCTTTTTATAAGTAGGAGTCTGGAGTAAATGATTTCCAATGGCTCTCAT TTCAATACAAAATTTCCGTTTATTAAATGCATGAGCTTCTGTTACTCCAAGACTGAGAAGGAAATTGAACCTGAGAC TCATTGACTGGCAAGATGTCCCCAGAGGCTCTCATTCAGCAATAAAATTCTCACCTTCACCCAGGCCCACTGAGTGT CAGATTTGCATGCACTAGTTCACGTGTGTAAAAAGGAGGATGCTTCTTTCCTTTGTATTCTCACATACCTTTAGGAA AGAACTTAGCACCCTTCCCACACAGCCATCCCAATAACTCATTTCAGTGACTCAACCCTTGACTTTATAAAAGTCTT GGGCAGTATAGAGCAGAGATTAAGAGTACAGATGCTGGAGCCAGACCACCTGAGTGATTAGTGACTCAGTTTCTCTT AGTAATTGTATGACTCAGTTTCTTCATCTGTAAAATGGAGGGTTTTTTAATTAGTTTGTTTTTGAGAAAGGGTCTCA CTCTGTCACCCAAATGGGAGTGTAGTGGCAAAATCTCGGCTCACTGCAACTTGCACTTCCCAGGCTCAAGCGGTCCT CCCACCTCAACATCCTGAGTAGCTGGAACCACAGGTACACACCACCATACCTCGCTAATTTTTTGTATTTTTGGTAG AGATGGGGTTTCACATGTTACACAGGATGGTCTCAGACTCCGGAGCTCAAGCAATCTGCCCACCTCAGCCTTCCAAA GTGCTGGGATTATAAGCATGATTACAGGAGTTTTAACAGGCTCATAAGATTGTTCTGCAGCCCGAGTGAGTTAATAC ATGCAAAGAGTTTAAAGCAGTGACTTATAAATGCTAACTACTCTAGAAATGTTTGCTAGTATTTTTTGTTTAACTGC AATCATTCTTGCTGCAGGTGAAAACTAGTGTTCTGTACTTTATGCCCATTCATCTTTAACTGTAATAATAAAAATAA CTGACATTTATTGAAGGCTATCAGAGACTGTAATTAGTGCTTTGCATAATTAATCATATTTAATACTCTTGGATTCT TTCAGGTAGATACTATTATTATCCCCATTTTACTACAGTTAAAAAAACTACCTCTCAACTTGCTCAAGCATACACTC TCACACACACAAACATAAACTACTAGCAAATAGTAGAATTGAGATTTGGTCCTAATTATGTCTTTGCTCACTATCCA ATAAATATTTATTGACATGTACTTCTTGGCAGTCTGTATGCTGGATGCTGGGGATACAAAGATGTTTAAATTTAAGC TCCAGTCTCTGCTTCCAAAGGCCTCCCAGGCCAAGTTATCCATTCAGAAAGCATTTTTTACTCTTTGCATTCCACTG TTTTTCCTAAGTGACTAAAAAATTACACTTTATTCGTCTGTGTCCTGCTCTGGGATGATAGTCTGACTTTCCTAACC TGAGCCTAACATCCCTGACATCAGGAAAGACTACACCATGTGGAGAAGGGGTGGTGGTTTTGATTGCTGCTGTCTTC AGTTAGATGGTTAACTTTGTGAAGTTGAAAACTGTGGCTCTCTGGTTGACTGTTAGAGTTCTGGCACTTGTCACTAT GCCTATTATTTAACAAATGCATGAATGCTTCAGAATATGGGAATATTATCTTCTGGAATAGGGAATCAAGTTATATT ATGTAACCCAGGATTAGAAGATTCTTCTGTGTGTAAGAATTTCATAAACATTAAGCTGTCTAGCAAAAGCAAGGGCT TGGAAAATCTGTGAGCTCCTCACCATATAGAAAGCTTTTAACCCATCATTGAATAAATCCCTATAGGGGATTTCTAC CCTGAGCAAAAGGCTGGTCTTGATTAATTCCCAAACTCATATAGCTCTGAGAAAGTCTATGCTGTTAACGTTTTCTT GTCTGCTACCCCATCATATGCACAACAATAAATGCAGGCCTAGGCATGACTGAAGGCTCTCTCATAATTCTTGGTTG CATGAATCAGATTATCAACAGAAATGTTGAGACAAACTATGGGGAAGCAGGGTATGAAAGAGCTCTGAATGAAATGG AAACCGCAATGCTTCCTGCCCATTCAGGGCTCCAGCATGTAGAAATCTGGGGCTTTGTGAAGACTGGCTTAAAATCA GAAGCCCCATTGGATAAGAGTAGGGAAGAACCTAGAGCCTACGCTGAGCAGGTTTCCTTCATGTGACAGGGAGCCTC CTGCCCCGAACTTCCAGGGATCCTCTCTTAAGTGTTTCCTGCTGGAATCTCCTCACTTCTATCTGGAAATGGTTTCT CCACAGTCCAGCCCCTGGCTAGTTGAAAGAGTTACCCATGCAGAGGCCCTCCTAGCATCCAGAGACTAGTGCTTAGA TTCCTACTTTCAGCGTTGGACAACCTGGATCCACTTGCCCAGTGTTCTTCCTTAGTTCCTACCTTCGACCTTGATCC TCCTTTATCTTCCTGAACCCTGCTGAGATGATCTATGTGGGGAGAATGGCTTCTTTGAGAAACATCTTCTTCGTTAG TGGCCTGCCCCTCATTCCCACTTTAATATCCAGAATCACTATAAGAAGAATATAATAAGAGGAATAACTCTTATTAT AGGTAAGGGAAAATTAAGAGGCATACGTGATGGGATGAGTAAGAGAGGAGAGGGAAGGATTAATGGATGATAAAATC TACTACTATTTGTTGAGACCTTTTATAGTCTAATCAATTTTGCTATTGTTTTCCATCCTCACGCTAACTCCATAAAA AAACACTATTATTATCTTTATTTTGCCATGACAAGACTGAGCTCAGAAGAGTCAAGCATTTGCCTAAGGTCGGACAT GTCAGAGGCAGTGCCAGACCTATGTGAGACTCTGCAGCTACTGCTCATGGGCCCTGTGCTGCACTGATGAGGAGGAT CAGATGGATGGGGCAATGAAGCAAAGGAATCATTCTGTGGATAAAGGAGACAGCCATGAAGAAGTCTATGACTGTAA ATTTGGGAGCAGGAGTCTCTAAGGACTTGGATTTCAAGGAATTTTGACTCAGCAAACACAAGACCCTCACGGTGACT TTGCGAGCTGGTGTGCCAGATGTGTCTATCAGAGGTTCCAGGGAGGGTGGGGTGGGGTCAGGGCTGGCCACCAGCTA TCAGGGCCCAGATGGGTTATAGGCTGGCAGGCTCAGATAGGTGGTTAGGTCAGGTTGGTGGTGCTGGGTGGAGTCCA TGACTCCCAGGAGCCAGGAGAGATAGACCATGAGTAGAGGGCAGACATGGGAAAGGTGGGGGAGGCACAGCATAGCA GCATTTTTCATTCTACTACTACATGGGACTGCTCCCCTATACCCCCAGCTAGGGGCAAGTGCCTTGACTCCTATGTT TTCAGGATCATCATCTATAAAGTAAGAGTAATAATTGTGTCTATCTCATAGGGTTATTATGAGGATCAAAGGAGATG CACACTCTCTGGACCAGTGGCCTAACAGTTCAGGACAGAGCTATGGGCTTCCTATGTATGGGTCAGTGGTCTCAATG TAGCAGGCAAGTTCCAGAAGATAGCATCAACCACTGTTAGAGATATACTGCCAGTCTCAGAGCCTGATGTTAATTTA GCAATGGGCTGGGACCCTCCTCCAGTAGAACCTTCTAACCAGCTGCTGCAGTCAAAGTCGAATGCAGCTGGTTAGAC TTTTTTTAATGAAAGCTTAGCTTTCATTAAAGATTAAGCTCCTAAGCAGGGCACAGATGAAATTGTCTAACAGCAAC TTTGCCATCTAAAAAAATCTGACTTCACTGGAAACATGGAAGCCCAAGGTTCTGAACATGAGAAATTTTTAGGAATC TGCACAGGAGTTGAGAGGGAAACAAGATGGTGAAGGGACTAGAAACCACATGAGAGACACGAGGAAATAGTGTAGAT TTAGGCTGGAGGTAAATGAAAGAGAAGTGGGAATTAATACTTACTGAAATCTTTCTATATGTCAGGTGCCATTTTAT GATATTTAATAATCTCATTACATATGGTAATTCTGTGAGATATGTATTATTGAACATACTATAATTAATACTAATGA TAAGTAACACCTCTTGAGTACTTAGTATATGCTAGAATCAAATTTAAGTTTATCATATGAGGCCGGGCACGGTGGCT CATATATGGGATTACATGCCTGTAATCCCAGCACTTTGGGAGGCCAAGGCAATTGGATCACCTGAGGTCAGGAGTTC CAGACCAGCCTGGCCAACATGGTGAAACCCCTTCTCTACTAAAAAATACAAAAAATCAGCCAGGTGTGGTGGCACGC GTCTATAATCCCAGCTACTCAGGAGGCTGAGGCAGGAGAATCACTTGAACCCAGGAGGTGGAGGTTGCAGTGAGCTA AGATTGCACCACTGCACTCCAGCCTAGGCGACAGAGTGAGACTCCATCTCAAAAAAAAAAAAAGAAGTTTATTATAT GAATTAACTTAGTTTTACTCACACCAATACTCAGAAGTAGATTATTACCTCATTTATTGATGAGGAGCCCAATGTAC TTGTAGTGTAGATCAACTTATTGAAAGCACAAGCTAATAAGTAGACAATTAGTAATTAGAAGTCAGATGGTCTGAGC TCTCCTACTGTCTACATTACATGAGCTCTTATTAACTGGGGACTCGAAAATCAAAGACATGAAATAATTTGTCCAAG CTTACAGAACCACCAAGTAGTAAGGCTAGGATGTAGACCCAGTTCTGCTACCTCTGAAGACAGTGTTTTTTCCACAG CAAAACACAAACTCAGATATTGTGGATGCGAGAAATTAGAAGTAGATATTCCTGCCCTGTGGCCCTTGCTTCTTACT TTTACTTCTTGGCGATTGGAAGTTGTGGTCCAAGCCACAGTTGCAGACCATACTTCCTCAACCATAATTGCATTTCT TCAGGAAAGTTTGAGGGAGAAAAAGGTAAAGAAAAATTTAGAAACAACTTCAGAATAAAGAGATTTTCTCTTGGGTT ACAGAGATTGTCATATGACAAATTATAAGCAGACACTTGAGAAAACTGAAGGCCCATGCCTGCCCAAATTACCCTTT GACCCCTTGGTCAAGCTGCAACTTTGGTTAAAGGGAGTGTTTATGTGTTATAGTGTTCATTTACTCTTCTGGTCTAA CCCATTGGCTCCGTCTTCATCCTGCAGTGACCTCAGTGCCTCAGAAACATACATATGTTTGTCTAGTTTAAGTTTGT GTGAAATTCTAACTAGCGTCAAGAACTGAGGGCCCTAAACTATGCTAGGAATAGTGCTGTGGTGCTGTGATAGGTAC ACAAGAAATGAGAAGAAACTGCAGATTCTCTGCATCTCCCTTTGCCGGGTCTGACAACAAAGTTTCCCCAAATTTTA CCAATGCAAGCCATTTCTCCATATGCTAACTACTTTAAAATCATTTGGGGCTTCACATTGTCTTTCTCATCTGTAAA AAGAATGGAAGAACTCATTCCTACAGAACTCCCTATGTCTTCCCTGATGGGCTAGAGTTCCTCTTTCTCAAAAATTA GCCATTATTGTATTTCCTTCTAAGCCAAAGCTCAGAGGTCTTGTATTGCCCAGTGACATGCACACTGGTCAAAAGTA GGCTAAGTAGAAGGGTACTTTCACAGGAACAGAGAGCAAAAGAGGTGGGTGAATGAGAGGGTAAGTGAGAAAAGACA AATGAGAAGTTACAACATGATGGCTTGTTGTCTAAATATCTCCTAGGGAATTATTGTGAGAGGTCTGAATAGTGTTG TAAAATAAGCTGAATCTGCTGCCTAACATTAACAGTCAAGAAATACCTCCGAATAACTGTACCTCCAATTATTCTTT AAGGTAGCATGCAACTGTAATAGTTGCATGTATATATTTATCATAATACTGTAACAGAAAACACTTACTGAATATAT ACTGTGTCCCTAGTTCTTTACACAATAAACTAATCTCATCCTCATAATTCTATTAGCTAATACATATTATCATCCTA TATTTCAGAGACTTCAAGAAGTTAAGCAACTTGCTCAAGATCATCTAAGAAGTAGGTGGTATTTCTGGGCTCATTTG GCCCCTCCTAATCTCTCATGGCAACATGGCTGCCTAAAGTGTTGATTGCCTTAATTCATCAGGGATGGGCTCATACT CACTGCAGACCTTAACTGGCATCCTCTTTTCTTATGTGATCTGCCTGACCCTAGTAGAACTTATGAAATTTCTGATG AGAAAGGAGAGAGGAGAAAGGCAGAGCTGACTGTGATGAGTGATGAAGGTGCCTTCTCATCTGGGTACCAGTGGGGC CTCTAAGACTAAGTCACTCTGTCTCACTGTGTCTTAGCCAGTTCCTTACAGCTTGCCCTGATGGGAGATAGAGAATG GGTATCCTCCAACAAAAAAATAAATTTTCATTTCTCAAGGTCCAACTTATGTTTTCTTAATTTTTAAAAAAATCTTG ACCATTCTCCACTCTCTAAAATAATCCACAGTGAGAGAAACATTCTTTTCCCCCATCCCATAAATACCTCTATTAAA TATGGAAAATCTGGGCATGGTGTCTCACACCTGTAATCCCAGCACTTTGGGAGGCTGAGGTGGGTGGACTGCTTGGA GCTCAGGAGTTCAAGACCATCTTGGACAACATGGTGATACCCTGCCTCTACAAAAAGTACAAAAATTAGCCTGGCAT GGTGGTGTGCACCTGTAATCCCAGCTATTAGGGTGGCTGAGGCAGGAGAATTGCTTGAACCCGGGAGGCGGAGGTTG CAGTGAGCTGAGATCGTGCCACTGCACTCCAGCCTGGGGGACAGAGCACATTATAATTAACTGTTATTTTTTACTTG GACTCTTGTGGGGAATAAGATACATGTTTTATTCTTATTTATGATTCAAGCACTGAAAATAGTGTTTAGCATCCAGC AGGTGCTTCAAAACCATTTGCTGAATGATTACTATACTTTTTACAAGCTCAGCTCCCTCTATCCCTTCCAGCATCCT CATCTCTGATTAAATAAGCTTCAGTTTTTCCTTAGTTCCTGTTACATTTCTGTGTGTCTCCATTAGTGACCTCCCAT AGTCCAAGCATGAGCAGTTCTGGCCAGGCCCCTGTCGGGGTCAGTGCCCCACCCCCGCCTTCTGGTTCTGTGTAACC TTCTAAGCAAACCTTCTGGCTCAAGCACAGCAATGCTGAGTCATGATGAGTCATGCTGAGGCTTAGGGTGTGTGCCC AGATGTTCTCAGCCTAGAGTGATGACTCCTATCTGGGTCCCCAGCAGGATGCTTACAGGGCAGATGGCAAAAAAAAG GAGAAGCTGACCACCTGACTAAAACTCCACCTCAAACGGCATCATAAAGAAAATGGATGCCTGAGACAGAATGTGAC ATATTCTAGAATATATTATTTCCTGAATATATATATATATATATATACACATATACGTATATATATATATATATATA TATTTGTTGTTATCAATTGCCATAGAATGATTAGTTATTGTGAATCAAATATTTATCTTGCAGGTGGCCTCTATACC TAGAAGCGGCAGAATCAGGCTTTATTAATACATGTGTATAGATTTTTAGGATCTATACACATGTATTAATATGAAAC AAGGATATGGAAGAGGAAGGCATGAAAACAGGAAAAGAAAACAAACCTTGTTTGCCATTTTAAGGCACCCCTGGACA GCTAGGTGGCAAAAGGCCTGTGCTGTTAGAGGACACATGCTCACATACGGGGTCAGATCTGACTTGGGGTGCTACTG GGAAGCTCTCATCTTAAGGATACATCTCAGGCCAGTCTTGGTGCATTAGGAAGATGTAGGCAACTCTGATCCTGAGA GGAAAGAAACATTCCTCCAGGAGAGCTAAAAGGGTTCACCTGTGTGGGTAACTGTGAAGGACTACAAGAGGATGAAA AACAATGACAGACAGACATAATGCTTGTGGGAGAAAAAACAGGAGGTCAAGGGGATAGAGAAGGCTTCCAGAAGAAT GGCTTTGAAGCTGGCTTCTGTAGGAGTTCACAGTGGCAAAGATGTTTCAGAAATGTGACATGACTTAAGGAACTATA CAAAAAGGAACAAATTTAAGGAGAGGCAGATAAATTAGTTCAACAGACATGCAAGGAATTTTCAGATGAATGTTATG TCTCCACTGAGCTTCTTGAGGTTAGCAGCTGTGAGGGTTTTGCAGGCCCAGGACCCATTACAGGACCTCACGTATAC TTGACACTGTTTTTTGTATTCATTTGTGAATGAATGACCTCTTGTCAGTCTACTCGGTTTCGCTGTGAATGAATGAT GTCTTGTCAGCCTACTTGGTTTCGCTAAGAGCACAGAGAGAAGATTTAGTGATGCTATGTAAAAACTTCCTTTTTGG TTCAAGTGTATGTTTGTGATAGAAATGAAGACAGGCTACATGATGCATATCTAACATAAACACAAACATTAAGAAAG GAAATCAACCTGAAGAGTATTTATACAGATAACAAAATACAGAGAGTGAGTTAAATGTGTAATAACTGTGGCACAGG CTGGAATATGAGCCATTTAAATCACAAATTAATTAGAAAAAAAACAGTGGGGAAAAAATTCCATGGATGGGTCTAGA AAGACTAGCATTGTTTTAGGTTGAGTGGCAGTGTTTAAAGGGTGATATCAGACTAAACTTGAAATATGTGGCTAAAT AACTAGAATACTCTTTATTTTTTCGTATCATGAATAGCAGATATAGCTTGATGGCCCCATGCTTGGTTTAACATCCT TGCTGTTCCTGACATGAAATCCTTAATTTTTGACAAAGGGGCTATTCATTTTCATTTTATATTGGGCCTAGAAATTA TGTAGATGGTCCTGAGGAAAAGTTTATAGCTTGTCTATTTCTCTCTCTAACATAGTTGTCAGCACAATGCCTAGGCT ATAGGAAGTACTCAAAGCTTGTTAAATTGAATTCTATCCTTCTTATTCAATTCTACACATGGAGGAAAAACTCATCA GGGATGGAGGCACGCCTCTAAGGAAGGCAGGTGTGGCTCTGCAGTGTGATTGGGTACTTGCAGGACGAAGGGTGGGG TGGGAGTGGCTAACCTTCCATTCCTAGTGCAGAGGTCACAGCCTAAACATCAAATTCCTTGAGGTGCGGTGGCTCAC TCCTGTAATCACAGCAGTTTGGGACGCCAAGGTGGGCAGATCACTTGAGGTCAGGAGTTGGACACCAGCCCAGCCAA CATAGTGAAACCTGGTCTCTGCTTAAAAATATAAAAATTAGCTGGACGTGGTGACGGGAGCCTGTAATCCAACTACT TGGGAGGCTGAGGCAGGAGAATCGCTTGAACCGGGGAGGTGGAGTTTGCACTGAGCAGAGATCATGCCATTGCACTC CAGCCTCCAGAGCGAGACTCTGTCTAAAGAAAAACGAAAACAAACAAACAAACAAACAAACAAAACCCATCAAATTC CCTGACCGAACAGAATTCTGTCTGATTGTTCTCTGACTTATCTACCATTTTCCCTCCTTAAAGAAACTGTGGAACTT CCTTCAGCTAGAGGGGCCTGGCTCAGAAGCCTCTGGTCAGCATCCAAGAAATACTTGATGTCACTTTGGCTAAAGGT ATGATGTGTAGACAAGCTCCAGAGATGGTTTCTCATTTCCATATCCACCCACCCAGCTTTCCAATTTTAAAGCCAAT TCTGAGGTAGAGACTGTGATGAACAAACACCTTGACAAAATTCAACCCAAAGACTCACTTTGCCTAGCTTCAAAATC CTTACTCTGACATATACTCACAGCCAGAAATTAGCATGCACTAGAGTGTGCATGAGTGCAACACACACACACACCAA TTCCATATTCTCTGTCAGAAAATCCTGTTGGTTTTTCGTGAAAGGATGTTTTCAGAGGCTGACCCCTTGCCTTCACC TCCAATGCTACCACTCTGGTCTAAGTCACTGTCACCACCACCTAAATTATAGCTGTTGACTCATAACAATCTTCCTG CTTCTACCACTGCCCCACTACAATTTCTTCCCAATATACTATCCAAATTAGTCTTTTCAAAATGTAAGTCATATATG GTCACCTCTTTGTTCAAAGTCTTCTGATAGTTTCCTATATCATTTATAATAAAACCAAATCCTTACAATTCTCTACA ATAGTTGTTCATGCATATATTATGTTTATTACAGATACGCATATATATAGCTCTCATATAAATAAATATATATATTT ATGTGTATGTGTGTAGAGTGTTTTTTCTTACAACTCTATGATGTAGGTATTATTAGTGTCCCAAATTTTATAATTTA GGACTTCTATGATCTCATCTTTTATTCTCCCCTTCACCGAATCTCATCCTACATTGGCCTTATTGATATTCCTTGAA AATTCTAAGCATCTTACATCTTTAGGGTATTTACATTTGCCATTCCCTATGCCCTAAATATTTAATCATAGTTTCAT ATAAATGGGTTCCTCATCATCTATGGGTACTCTCTCAGGTGTTAACTTTATAGTGAGGACTTTCCTGCCATACTACT TAAAGTAGCGATACCCTTTCACCCTGTCCTAATCACACTCTGGCCTTCATTTCAGTTTTTTTTTTTTCTCCATAGCA CCTAATCTCATTGGTATATAACATGTTTCATTTGCTTATTTAATGTCAAGCTCTTTCCACTATCAAGTCCATGAAAA CAGGAACTTTATTCCTCTATTCTGTTTTTGTGCTGTATTCTTAGCAATTTTACAATTTTGAATGAAATGAATGAGCA GTCAAACACATATACAACTATAATTAAAAGGATGTATGCTGACACATCCACTGCTATGCACACACAAAGAAATCAGT GGAGTAGAGCTGGAAGCGCTAAGCCTGCATAGAGCTAGTTAGCCCTCCGCAGGCAGAGCCTTGATGGGATTACTGAG TTCTAGAATTGGACTCATTTGTTTTGTAGGCTGAGATTTGCTCTTGAAAACTTGTTCTGACCAAAATAAAAGGCTCA AAAGATGAATATCGAAACCAGGGTGTTTTTTACACTGGAATTTATAACTAGAGCACTCATGTTTATGTAAGCAATTA ATTGTTTCATCAGTCAGGTAAAAGTAAAGAAAAACTGTGCCAAGGCAGGTAGCCTAATGCAATATGCCACTAAAGTA AACATTATTCCATAGGTGTCAGATATGGCTTATTCATCCATCTTCATGGGAAGGATGGCCTTGGCCTGGACATCAGT GTTATGTGAGGTTCAAAACACCTCTAGGCTATAAGGCAACAGAGCTCCTTTTTTTTTTTTCTGTGCTTTCCTGGCTG TCCAAATCTCTAATGATAAGCATACTTCTATTCAATGAGAATATTCTGTAAGATTATAGTTAAGAATTGTGGGAGCC ATTCCGTCTCTTATAGTTAAATTTGAGCTTCTTTTATGATCACTGTTTTTTTAATATGCTTTAAGTTCTGGGGTACA TGTGCCATGGTGGTTTGCTGCACCCATCAACCCGTCATCTACATTAGGTATTTCTCCTAATGCTATCCTTCCCCTAG CCCCCCACCCCCAACAGGCCCCAGTGTGTGATGTTCCCCTCCCTGTGTCCATGGATCACTGGTTTTTTTTTTTTTTT TTTTTTTTTTTTTAAAGTCTCAGTTAAATTTTTGGAATGTAATTTATTTTCCTGGTATCCTAGGACCTGCAAGTTAT CTGGTCACTTTAGCCCTCACGTTTTGATGATAATCACATATTTGTAAACACAACACACACACACACACACACACACA TATATATATATAAAACATATATATACATAAACACACATAACATATTTATCGGGCATTTCTGAGCAACTAACTCATGC AGGACTCTCAAACACTAACCTATAGCCTTTTCTATGTATCTACTTGTGTAGAAACCAAGCGTGGGGACTGAGAAGGC AATAGCAGGAGCATTCTGACTCTCACTGCCTTTGGCTAGGTCCCTCCCTCATCACAGCTCAGCATAGTCCGAGCTCT TATCTATATCCACACACAGTTTCTGACGCTGCCCAGCTATCACCATCCCAAGTCTAAAGAAAAAAATAATGGGTTTG CCCATCTCTGTTGATTAGAAAACAAAACAAAATAAAATAAGCCCCTAAGCTCCCAGAAAACATGACTAAACCAGCAA GAAGAAGAAAATACAATAGGTATATGAGGAGACTGGTGACACTAGTGTCTGAATGAGGCTTGAGTACAGAAAAGAGG CTCTAGCAGCATAGTGGTTTAGAGGAGATGTTTCTTTCCTTCACAGATGCCTTAGCCTCAATAAGCTTGCGGTTGTG GAAGTTTACTTTCAGAACAAACTCCTGTGGGGCTAGAATTATTGATGGCTAAAAGAAGCCCGGGGGAGGGAAAAATC ATTCAGCATCCTCACCCTTAGTGACACAAAACAGAGGGGGCCTGGTTTTCCATATTTCCTCATGATGGATGATCTCG TTAATGAAGGTGGTCTGACGAGATCATTGCTTCTTCCATTTAAGCCTTGCTCACTTGCCAATCCTCAGTTTTAACCT TCTCCAGAGAAATACACATTTTTTATTCAGGAAACATACTATGTTATAGTTTCAATACTAAATAATCAAAGTACTGA AGATAGCATGCATAGGCAAGAAAAAGTCCTTAGCTTTATGTTGCTGTTGTTTCAGAATTTAAAAAAGATCACCAAGT CAAGGACTTCTCAGTTCTAGCACTAGAGGTGGAATCTTAGCATATAATCAGAGGTTTTTCAAAATTTCTAGACATGA GATTCAAAGCCCTGCACTTAAAATAGTCTCATTTGAATTAACTCTTTATATAAATTGAAAGCACATTCTGAACTACT TCAGAGTATTGTTTTATTTCTATGTTCTTAGTTCATAAATACATTAGGCAATGCAATTTAATTAAAAAAACCCAAGA ATTTCTTAGAATTTTAATCATGAAAATAAATGAAGGCATCTTTACTTACTCAAGGTCCCAAAAGGTCAAAGAAACCA GGAAAGTAAAGCTATATTTCAGCGGAAAATGGGATATTTATGAGTTTTCTAAGTTGACAGACTCAAGTTTTAACCTT CAGTGCCCATGATGTAGGAAAGTGTGGCATAACTGGCTGATTCTGGCTTTCTACTCCTTTTTCCCATTAAAGATCCC TCCTGCTTAATTAACATTCACAAGTAACTCTGGTTGTACTTTAGGCACAGTGGCTCCCGAGGTCAGTCACACAATAG GATGTCTGTGCTCCAAGTTGCCAGAGAGAGAGATTACTCTTGAGAATGAGCCTCAGCCCTGGCTCAAACTCACCTGC AAACTTCGTGAGAGATGAGGCAGAGGTACACTACGAAAGCAACAGTTAGAAGCTAAATGATGAGAACACATGGACTC ATAGAGGGAAACAACGCATACTGGGGCCTATCAGAGGGTGGAGGGTGAGAGAAGGAGAGGATCAGGAAAAATCACTA ATGGATGCTAAGCGTAATACCTGAGTGATGAGATCATCTATACAACAAACCCCCTTGACATTCATTTATCTATGTAA CAAACCTGCACATCCTGTACACGTACCCCTGAACTTAAAATAAAAGTTGAAAACAAGAAAGCAACAGTTTGAACACT TGTTATGGTCTATTCTCTCATTCTTTACAATTACACTAGAAAATAGCCACAGGCTCCTGCAAGGCAGCCACAGAATT TATGACTTGTGATATCCAAGTCATTCCTGGATAATGCAAAATCTAACACAAAATCTAGTAGAATCATTTGCTTACAT CTATTTTTGTTCTGAGAATATAGATTTAGATACATAATGGAAGCAGAATAATTTAAAATCTGGCTAATTTAGAATCC TAAGCAGCTCTTTTCCTATCAGTGGTTTACAAGCCTTGTTTATATTTTTCCTATTTTAAAAATAAAAATAAAGTAAG TTATTTGTGGTAAAGAATATTCATTAAAGTATTTATTTCTTAGATAATACCATGAAAAACATTCAGTGAAGTGAAGG GCCTACTTTACCCAACAAGAATCTAATTTATATAATTTTTCATACTAATAGCATCTAAGAACAGTACAATATTTGAC TCTTCAGGTTAAACATATGTCATAAATTAGCCAGAAAGATTTAAGAAAATATTGGATGTTTCCTTGTTTAAATTAGG CATCTTACAGTTTTTAGAATCCTGCATAGAACTTAAGAAATTACAAATGCTAAAGCAAACCCAAACAGGCAGGAATT AATCTTCATCGAATTTGGGTGTTTCTTTCTAAAAGTCCTTTATACTTAAATGTCTTAAGACATACATAGATTTTATT TTACTAATTTTAATTATACAGACAATAAATGAATATTCTTACTGATTACTTTTTCTGACTGTCTAATCTTTCTGATC TATCCTGGATGGCCATAACACTTATCTCTCTGAACTTTGGGCTTTTAATATAGGAAAGAAAAGCAATAATCCATTTT TCATGGTATCTCATATGATAAACAAATAAAATGCTTAAAAATGAGCAGGTGAAGCAATTTATCTTGAACCAACAAGC ATCGAAGCAATAATGAGACTGCCCGCAGCCTACCTGACTTCTGAGTCAGGATTTATAAGCCTTGTTACTGAGACACA AACCTGGGCCTTTCAATGCTATAACCTTTCTTGAAGCTCCTCCCTACCACCTTTAGCCATAAGGAAACATGGAATGG GTCAGATCCCTGGATGCAAGCCAGGTCTGGAACCATAGGCAGTAAGGAGAGAAGAAAATGTGGGCTCTGCAACTGGC TCCGAGGGAGCAGGAGAGAATCAACCCCATACTCTGAATCTAAGAGAAGACTGGTGTCCATACTCTGAATGGGAAGA ATGATGGGATTACCCATAGGGCTTGTTTTAGGGAGAAACCTGTTCTCCAAACTCTTGGCCTTGAGATACCTGGTCCT TATTCCTTGGACTTTGGCAATGTCTGACCCTCACATTCAAGTTCTGAGGAAGGGCCACTGCCTTCATACTGTGGATC TGTAGCAAATTCCCCCTGAAAACCCAGAGCTGTATCTTAATTGTTTAAAAAAATTATATTATCTCAAGGACTGTTCT TCTCTGAGTAGCCAAGCTCAGCTTGGTTCAAGCTACAAGCAGCTGAGCTGCTTTTTGTCTAGTCATTGTTCTTTTAT TTCAGTGGATCAAATACGTTCTTTCCAAACCTAGGATCTTGTCTTCCTAGGCTATATATTTTGTCCCAGGAAGTCTT AATCTGGGGTCCACAGAACACTAGGGGGCTGGTGAAGTTTATAGAAAAAAAATCTGTATTTTTACTTACATGTAACT GAAATTTAGCATTTTCTTCTACTTTGAATGCAAAGGACAAACTAGAATGACATCATCAGTACCTATTGCATAGTTAT AAAGAGAAACCACAGATATTTTCATACTACACCATAGGTATTGCAGATCTTTTTGTTTTTGTTTTTGTTTGAGATGG AGTTTCGCTCTTATTGCCCAGGCTGGAGTGCAGTGGCATGATTTCGGCTCACTGCAACCTCCCCTTCCTGCATTCAA GCAATTCTCCTGCCTTGGCCTCCAGAGTAGCTGGGGATTACAGGCACCTGCCACCATGCCAGTCTAATTTTTGTATT TTTAGTAGAGATGGGGTTTCGCCATGTTGGCCAGGCTGGTCTTGAACTCCTGACCTCAGATGATCTGCCCGCCTTGG CCTCCTGAAGTGCTGGGATTATAGGTGTGAGCCACCACGCCTGGCCCATTGCAGATATTTTTAATTCACATTTATCT GCATCACTACTTGGATCTTAAGGTAGCTGTAGACCCAATCCTAGATCTAATGCTTTCATAAAGAAGCAAATATAATA AATACTATACCACAAATGTAATGTTTGATGTCTGATAATGATATTTCAGTGTAATTAAACTTAGCACTCCTATGTAT ATTATTTGATGCAATAAAAACATATTTTTTTTAGCACTTACAGTCTGCCAAACTGGCCTGTGACACAAAAAAAGTTT AGGAATTCCTGGTTTTGTCTGTGTTAGCCAATGGTTAGAATATATGCTCAGAAAGATACCATTGGTTAATAGCTGAA AGAAAATGGAGTAGAAATTCAGTGGCCTGGAATAATAACAATTTGGGCAGTCATTAAGTCAGGTGAAGACTTCTGGA ATCATGGGAGAAAAGCAAGGGAGACATTCTTACTTGCCACAAGTGTTTTTTTTTTTTTTTTTTTATCACAAACATAA GAAAATATAATAAATAACAAAGTCAGGTTATAGAAGAGAGAAACGCTCTTAGTAAACTTGGAATATGGAATCCCCAA AGGCACTTGACTTGGGAGACAGGAGCCATACTGCTAAGTGAAAAAGACGAAGAACCTCTAGGGCCTGAACATAGGAA ATTGTAGGAACAGAAATTCCTAGATCTGGTGGGGCAAGGGGAGCCATAGGAGAAAGAAATGGTAGAAATGGATGGAG ACGGAGGCAGAGGTGGGCAGATCATGAGGTCAAGAGATCGAGACCATCCTGGCAAACATGGTGAAATCCCGTCTCTA CTAAAAATAAAAAAATTAGCTGGGCATGGTGGCATGCGCCTGTAGTCCCAGCTGCTCGGGAGGCTGAGGCAGGAGAA TCGTTTGAACCCAGGAGGCGAAGGTTGCAGTGAGCTGAGATAGTGCCATTGCACTCCAGTCTGGCAACAGAGTGAGA CTCCGTCTCAAAAAAAAAAAAAAGAAAGAAAGAAAAGAAAAAGAAAAAAGAAAAAATAAATGGATGTAGAACAAGCC AGAAGGAGGAACTGGGCTGGGGCAATGAGATTATGGTGATGTAAGGGACTTTTATAGAATTAACAATGCTGGAATTT GTGGAACTCTGCTTCTATTATTCCCCCAATCATTACTTCTGTCACATTGATAGTTAAATAATTTCTGTGAATTTATT CCTTGATTCTAAAATATGAGGATAATGACAATGGTATTATAAGGGCAGATTAAGTGATATAGCATAAGCAATATTCT TCAGGCACATGGATCGAATTGAATACACTGTAAATCCCAACTTCCAGTTTCAGCTCTACCAAGTAAAGAGCTAGCAA GTCATCAAAATGGGGACATACAGAAAAAAAAAAGGACACTAGAGGAATAATATACCCTGACTCCTAGCCTGATTAAT ATATCGATTCACTTTTTTCTCTGTTTGATGACAAATTCTGGCTTTAAATAATTTTAGGATTTTAGGCTTCTCAGCTC CCTTCCCAGTGAGAAGTATAAGCAGGACAGACAGGCAAGCAAGAAGAGAGCCCCAGGCAATACTCACAAAGTAGCCA GTGTCCCCTGTGGTCATAGAGAAATGAAAAGAGAGAGGATTCCCTGGAAGCACTGGATGTAATCTTTTCTGTCTGTC CTCTCTAGGGAATCACCCCAAGGTACTGTACTTTGGGATTAAGGCTTTAGTCCCACTGTGGACTACTTGCTATTCTG TTCAGTTTCTAGAAGGAACTATGTACGGTTTTTGTCTCCCTAGAGAAACTAAGGTACAGAAGTTTTGTTTACAATGC ACTCCTTAAGAGAGCTAGAACTGGGTGAGATTCTGTTTTAACAGCTTTATTTTCTTTTCCTTGGCCCTGTTTTTGTC AACTGTCACCACCTTTAAGGCAAATGTTAAATGTGCTTTGGCTGAAACTTTTTTTCCTATTTTGAGATTTGCTCCTT TATATGAGGCTTTCTTGGAAAAGGAGAATGGGAGAGATGGATATCATTTTGGAAGATGATGAAGAGGGTAAAAAAGG GGACAAATGGAAATTTGTGTTGCAGATAGATGAGGAGCCAACAAAAAAGAGCCTCAGGATCCAGCACACATTATCAC AAACTTAGTGTCCATCCATCACTGCTGACCCTCTCCGGACCTGACTCCACCCCTGAGGACACAGGTCAGCCTTGACC AATGACTTTTAAGTACCATGGAGAACAGGGGGCCAGAACTTCGGCAGTAAAGAATAAAAGGCCAGACAGAGAGGCAG CAGCACATATCTGCTTCCGACACAGCTGCAATCACTAGCAAGCTCTCAGGCCTGGCATCATGGTGCATTTTACTGCT GAGGAGAAGGCTGCCGTCACTAGCCTGTGGAGCAAGATGAATGTGGAAGAGGCTGGAGGTGAAGCCTTGGGCAGGTA AGCATTGGTTCTCAATGCATGGGAATGAAGGGTGAATATTACCCTAGCAAGTTGATTGGGAAAGTCCTCAAGATTTT TTGCATCTCTAATTTTGTATCTGATATGGTGTCATTTCATAGACTCCTCGTTGTTTACCCCTGGACCCAGAGATTTT TTGACAGCTTTGGAAACCTGTCGTCTCCCTCTGCCATCCTGGGCAACCCCAAGGTCAAGGCCCATGGCAAGAAGGTG CTGACTTCCTTTGGAGATGCTATTAAAAACATGGACAACCTCAAGCCCGCCTTTGCTAAGCTGAGTGAGCTGCACTG TGACAAGCTGCATGTGGATCCTGAGAACTTCAAGGTGAGTTCAGGTGCTGGTGATGTGATTTTTTGGCTTTATATTT TGACATTAATTGAAGCTCATAATCTTATTGGAAAGACCAACAAAGATCTCAGAAATCATGGGTCGAGCTTGATGTTA GAACAGCAGACTTCTAGTGAGCATAACCAAAACTTACATGATTCAGAACTAGTGACAGTAAAGGACTACTAACAGCC TGAATTGGCTTAACTTTTCAGGAAATCTTGCCAGAACTTGATGTGTTTATCCCAGAGAATTGTATTATAGAATTGTA GACTTGTGAAAGAAGAATGAAATTTGGCTTTTGGTAGATGAAAGTCCATTTCAAGGAAATAGAAATGCCTTATTTTA TGTGGGTCATGATAATTGAGGTTTAGAAGAGATTTTTGCAAAAAAAATAAAAGATTTGCTCAAAGAAAAATAAGACA CATTTTCTAAAATATGTTAAATTTCCCATCAGTATTGTGACCAAGTGAAGGCTTGTTTCCGAATTTGTTGGGGATTT TAAACTCCCGCTGAGAACTCTTGCAGCACTCACATTCTACATTTACAAAAATTAGACAATTGCTTAAAGAAAAACAG GGAGAGAGGGAACCCAATAATACTGGTAAAATGGGGAAGGGGGTGAGGGTGTAGGTAGGTAGAATGTTGAATGTAGG GCTCATAGAATAAAATTGAACCTAAGCTCATCTGAATTTTTTGGGTGGGCACAAACCTTGGAACAGTTTGAGGTCAG GGTTGTCTAGGAATGTAGGTATAAAGCCGTTTTTGTTTGTTTGTTTGTTTTTTCATCAAGTTGTTTTCGGAAACTTC TACTCAACATGCCTGTGTGTTATTTTGTCTTTTGCCTAACAGCTCCTGGGTAACGTGATGGTGATTATTCTGGCTAC TCACTTTGGCAAGGAGTTCACCCCTGAAGTGCAGGCTGCCTGGCAGAAGCTGGTGTCTGCTGTCGCCATTGCCCTGG CCCATAAGTACCACTGAGTTCTCTTCCAGTTTGCAGGTCTTCCTGTGACCCTGACACCCTCCTTCTGCACATGGGGA CTGGGCTTGGCCTTGAGAGAAAGCCTTCTGTTTAATAAAGTACATTTTCTTCAGTAATCAAAAATTGCAATTTTATC TTCTCCATCTTTTACTCTTGTGTTAAAAGGAAAAAGTGTTCATGGGCTGAGGGATGGAGAGAAACATAGGAAGAACC AAGAGCTTCCTTAAGAAATGTATGGGGGCTTGTAAAATTAATGTGGATGTTATGGGAGAATTCCCAAGATTCCCAAG GAGGATGATATGATGGAGAAAAATCTTTATCGGGGTGGGAAAATGGTTAATTAAGTGGCAGAGACTCCTAGGCAGTT TTTACTGCACCGGGGAAAGAAGGAGCTGTTGTGGTACCTGAGAAAGCAGATTTGTGGTACATGTCACTTTTCATTAA AAACAAAAACAAAACAAAACAAAACTTCATAGATATCCAAGATATAGGCTGAGAATTACTATTTTAATTTACTCTTA TTTACATTTTGAAGTAGCTAGCTTGTCACATGTTTTATGAAATTGATTTGGAGATAAGATGAGTGTGTATCAACAAT AGCCTGCTCTTTCCATGAAGGATTCCATTATTTCATGGGTTAGCTGAAGCTAAGACACATGATATCATTGTGCATTA TCTTCTGATACAATGTAACATGCACTAAAATAAAGTTAGAGTTAGGACCTGAGTGGGAAAGTTTTTGGAGAGTGTGA TGAAGACTTTCCGTGGGAGATAGAATACTAATAAAGGCTTAAATTCTAAAACCAGCAAGCTAGGGCTTCGTGACTTG CATGAAACTGGCTCTCTGGAAGTAGAAGGGAGAGTAAGACATACGTAGAGGACTAGGAAAGACCAGATAGTACAGGG CCTGGCTACAAAAATACAAGCTTTTACTATGCTATTGCAATACTAAACGATAAGCATTAGGATGTTAAGTGACTCAG GAAATAAGATTTTGGGAAAAAGTAATCTGCTTATGTGCACAAAATGGATTCAAGTTTGCAGATAAAATAAAATATGG ATGATGATTCAAGGGGACAGATACAATGGTTCAAACCCAAGAGGAGCAGTGAGTCTGTGGAATTTTGAAGGATGGAC AAAGGTGGGGTGAGAAAGACATAGTATTCGACCTGACTGTGGGAGATGAGAAGGAAGAAGGAGGTGATAAATGACTG AAAGCTCCCAGACTGGTGAAGATAACAGGAGGAAACCATGCACTTGACCCTGGTGACTCTCATGTGTGAAGGGTAGA GGGATATTAACAGATTTACTTTTTAGGAAGTGCTAGATTGGTCAGGGAGTTTTGACCTTCAGGTCTTGTGTCTTTCA TATCAAGGAACCTTTGCATTTTCCAAGTTAGAGTGCCATATTTTGGCAAATATAACTTTATTAGTAATTTTATAGTG CTCTCACATTGATCAGACTTTTTCCTGTGAATTACTTTTGAATTTGGCTGTATATATCCAGAATATGGGAGAGAGAC AAATAATTATTGTAGTTGCAGGCTATCAACAATACTGGTCTCTCTGAGCCTTATAACCTTTCAATATGCCCCATAAA CAGAGTAAACAGGGATTATTCATGGCACTAAATATTTTCACCTAGGTCAGTCAACAAATGGAGGCAATGTGCATTTT TTGATACATATTTTTATATATTTATGGGGCATGTGATACTTACATGCCTAGAACATGTGACTGATTAAGTCTAGATA TTTAGGATATCCATTACTTTGAGCATTTATCATTTCTATGTATTGAGAAAATTTCAAATCCTCATTTCTGACCATTT TGAAATATATAATAAATAGTAATTAACTATAGTCACCCTACTCAAATATCAACATTATAAACTAACTAATCCTTCTT TCCACTTTTTTACCAACCAACATCTCTTAAATCCCCTGCCATACACATCACACATTTTTCAGCTCTGATAACTATCA TTCTACTCTCATACCACCATGAGACCACTTTTTTAGCTCCACAGATGAATAAAAACATGTGATATTTGACTTTCTGT ATCTGGCTTATTTTATTATCTATCTCTTTGGCATACCAAGAGTTTGTTTTTGTTCTGCTTCAGGGCTTTCAATTAAC ATAATGACCTCTGGTTCCATCCATGTTGCTACAAATGACAAGATTTCATTCTTTTTCATGGCAAAATAGTACTGTGC AAAAAATACAATTTTTTAATCCGTTCATCTGTTGATAGACACTTAGGTTGATCCCAAACCTTAACTATTGTGAATAG GTGCTTCAATAAACATGAGTGTAATGTGTCCATTGGATATACTGATTTCCTTTCTTTTGGATAAATAACCACTAGTG AGATTGCTGGATTGTATGATAGTTCTGTTTTTAGTTTATTGAGAAATCTTCATACTGTTTTCCATAATGGTTGTACT ATTTTACATTCCCACCAACAGTGTGTAAGAAAGAGTTCCCTTTTCTCCATATCCTCACAAGGATCTGTTATTTTTTG TCTTTTTTGTTAATAGCATTTTAACTAGAGTAAGTAGATATCTCATTGTAGTTTTGATTTGCATTTCCCTGATCATT AGTGATGTTGAGATTTTTTCATATGTTTGTTGGTCATTTGTATATCTTTTTCTGAGATTGTCTGTTCATGTCCTTAT CCTACTTTTATTGGGATTGTTGTTATTTTCTTGATAATCATTGTGTCATTTTAGAGCCTGGATATTATTCTTTTGTC AGATGTATAGATTGTGAAGATTTTCTCCTCTGTGGGTTGTCTGTTTATTCTGCAGACTCTTCCTTTTGCCATGCAAA AGCTCTTTAGTTTAATTTAGTCCCAGATATTTTCTTTGTTTTTATGTGTTTGCATTTGTGTTCTTGTCATGAAATCC TTTCCTAAGCCAATGTGTAGAAGGGTTTTTCCGATGTTATTTTCTAGAATTGTTACAGTTTCAGGCTTAGATTTAAG TCCTTGATCCATCTTAAGTTGATTTTTGTATAAGGTGAGAGATGAAGATCCAGTTTCATTCTCCTACATGTAGCTTG CCAGCTATCCCGACTCATTTGTTGAATAGGGTGCCCTTTCCCATTTATGTTTTTGTTTGCTTTGTCAAAGATCAGTT CGGATGTAAGTATTTGAGTTTATTTCTGGGTTCTCTATTCTGTTCCATTGGTCCGATGTGCCTATTTGTACACCAGC ATCATGCTGTGTTTTTGGTGACTATGGCCTTATTGTATAGTTTGAAATGAGGTAATGTAATGCCATTCAGATTTGTT CTTTTTTTTAGACTTGCTTGTTTATTGGGCTCTTTTTTGGTTCCATAAGAATTTTAGGATTGTTTTTTCTAGTTCTG TGAAGGCTAATGGTGGTATTTATGGGAATTGCAATGCAATTTGTAGGTTGCTTCTGGCATTATGGCCATTTTCACAA TATTGATTCTACCCATCTATGAGAATGGCATGTGTTTCCATTTGTTTGTGTCTTATATGATTACTATCAGCCGTGTT TTGTAGTTTTCCTTGTAGATGTCTTTCACCTCCTTGGTTAGGTATATATTCCTAAGTTTTTGTTTTGTTTTGTTTTG TTTTTTGCAGCTATTGTAAAAGGGGTTGAGTTATTGATTTTATTCTCATCTTGGTCATTGCTGGTATGTAAGAAAGC AACTCATTGGTGTACGTTAATTTTGTATCCAGAAACTTTGCTGAATTATTTTATCAGTTCTAGGGGGTTTTGGAGGA GTCTTTAGAGTTTTCTACATACACAATCATATCATCAGCAAACAGTGACAGTTTGACTTTCTCTTTAACAATTTGGA TGTGCTTTACTTGTTTCTCTTGTCTGATTGCTCTTGCTAGGACTTCCAGTAATATGTTAAAGAGAAGTGGTGAGAGT GGGTATCCTTGTCTCATTCCAGTTTTCAGACAGAATGCTTTTAACTTTTTCCCATTCAATATAATGTTGGCTGTGTG TTTACCATAGCTGGCTTTTATTACATTGAGGTATGTCCTTTGTAAACCGATTTTGCTGAGTTTTAGTCATAAAGTGA TGTTGAATTTTGTTGAATGCAGTTTCTGTGGCTATTGAGATAATCACATGATTTTTGTTTCCAATTCTCTTTATGTT GTGTATCACACTTATTGACTTGCGTATGTTAAACCATCCGTGCATCCCTCGCATGAAACCACTTGATCATGGGTTTT GATATGCCGTGTGGGATGCTATTAGCTATATTTTGTCAAGGATGTTGGCATCTATGTTCATCAGGGATATTGATCTG TAGTGTTTTTTTTTTTTGGTTATGTTCTTTCCCAGTTTTGGTATTAAGGTGATACTGGCTTCATAGAATGATTTAGG GAGGATTCTCTCTTTCTCTATCTTGTAGAATACTGTCAATAGGATTGGTATCAATTCTTCTTTGAATGTCTGGTAGA ATTCGAACGTCTCCTTTAGGTTTTCTAGTTTATTCATGTAAAGGTGTTCATAGTAACCTTGAATAATCTTTTGTATT TCTGTGGTATCAGTAATAGTATCTCCTGTTTTGTTTCTAACTGAGTTTATTTGCACTTCTCTCCTCTTTTCTTGGTT AATCTTGCTAATGGTCTATCAGTTTTATTTATCTTTTCAAAGAACCAGCTTTTTATTTCATTTAGCTTTTGTATTTT TTTGCAGTTGTTTTAATTTCATTTAGTTCTCCTCTTATCTTAGTTATTCCCTTTCTTTTGCTGGGTTTTGGTTCTGT TTGTTTTTGTTTCTCTAGTTTCTTGTGGTGTGACCTTATATTGTCTGTCCTCTTTCAGACTCTTTGACATCGACATT TAGGGCTGTGAACTTTCCTTTTAGCACCATCTTTGCTGTATCCTAGAGGTTTTGATAGGTGTGTCACTATTGTCGGT CAGTTCAAGTAATTTTGTTGTTCTTATTATACTTTAAGTTCTGGGATACATGTGCAGAATGTGCAGGTTTGTTACAT AGGTATAGATGTGCCATGGTGGTTTGCTGCTCCCATCAACCTGTCATCTACATTAGGTATTTCTTTTAATGTTATCC CTCTCCTAACCCCCTCACCCCCCGACAGGCCCTGGTGTGTGATGTTCCCCTCCCTGTGTCCATGTGTTCTCATTGTT CAACTCCCACTTATGAGTGAGAACGTGTGGTGTTTGGTTTCTCTGTTCCTGTGTTAGTTTGCTCAGAATGATGTTTC CACCTTCACCATGTCCCTGCAAAGACATGAACTCATCATTTTATGGCTGCATATATTCCATGGTGTATATGTGCCAC ATTTTCTTTATCCATTATATCGCTGATGGCCATTTGGGTTGGTTCCAAGTCTTTGGTATTGTGAATAGTGCCGCAAT AAACATACGTGTGCACATGTCTTTATAGTAGAATGATTTCTAATTCTTTGGGTATATACCCAGTAATGGGATTGCTG GGTCAAACAGTATTTCTGGTTCTAGATCCTTGAGGAATTGCCACACTGTCTTCCACAATGGTTGAACTAATTTACAC ACCCATCAACAGTGTAAAATTTTTCCTATTCTTCCACATCCTCTCCAGCACCTTTTGTTTCCTGACTTTTTAATAAT TGCCATTCTAACTGGCATGAGATGGTATCTCATTGTGGTTTTGATTTGCATTTCTCTAATGACCAGTGATGATGAGC TTCTTTTCATGTGTTTCTTGGCCACATAAATGACTTCTTTAGAGAAGCATCTGTTCATATCCTTTGTCCACTTTTTG ATGGGGTCGTTAGGTTTTTTCTTGTAAATTTGTTGAAGTTCTTTGTAGATTTTGGATGTTAGCCCTTTGTCAGATGG ATAGATTGGCAAAAATTTTCTCCCATTCTGTAGGTTGCCTGTTCACTCTGATGATAGTCTTTTGCTGTGCAGAAGCT CTTTAGTTTAATTAGATCCCATATGTCAATTTTGGCCTTTGTTGTCATTGCTTTTGATGTTTAGTCGTGGAATTTTG CCCATGCCTATGTCCTGAATGGTATTGCCTAGGTTATCTTCTAGGATTTTTATGGTTTTAGGTTGCACATTTAAGTC TTTAATCCACCTTGAGTTAATTTTTGTATAAGGTGTAAGGAAGGGGTACAGTTTCAGTTTTATGCATATTGCTAGCC AGTTTTTCCAGCACCATTTATTAAATAGGGAATTCTTTCTCCATTGCTTTTGTGATGTTTGTCAAAGATCAGATGGT CGTAGATGTGTGGCATTATTTCTGAGGCTTCTGTTCTGTTCCACTGGTCTATATATCTGTTTTGGTACCAGTACCAT GCTGTTTTTGTTACTGTAGCCTTGTAGTATAGCTTGAAGTCAGGTAGCATCATGCCTCCAGCTTTGTTCTTTTTGTT TAGGATTGTCTTGGCTATATGGGCTCTTTTTTGATTCCATATGACATTTAAAGTAGTTTTTTCTAATTCTTTGAAAA AAGTCAGTGGTAGCTTGATGGGGATAGCATTGAATCTATAAATTACTTTGGGCAGTATGGCCATTTTAAAGATATTG ATTCTTTCTATCTATGAGCATGGAATGTTTTTCCATTTGTTTGTGTCCTCTCTTATTTCCTTGAGCAGTGAGTGGTT TGTAGCTCTCCTTGAAGAGGTTCTTCACATCCCTTATAAGTTGTATTTCTAGGTATTTTATTTTATTCTCTTTGCAG CAATTGTGAATGGGAGTTCACCCATGATTTGGCTCTCTGCTTGTCTATTATTGGTGTATAGGAATGCTTGTGATTTT TGCACACTGATTTTGTATCTTGAGACTTTGCTGAAGCTGTTTATCAGCTTAAGATTTTGGGCTGAGATGACAGGGTC TTCTAAATATACAATCATGTCATCTGCAAACAGAGACAATTTGACTTCCTCTCTTCCTATTTGAATATGCTTTATTT CTTTCTCTTGCCTGATTGTCCTGGCGAGAACTTCCAATACTATGTTGAGTAAGAGTGGCGAGAGGGCATCCTTGTCT TGTGCCGGTTTTCAAAGCAAATGATTTTTAAATTTCCGTCTTGATTTCATTGTTGACCCAATGATCATTCAGGAGCA GGTTATTTAATTTCCCTGTATTTGCATGGTTTTGAAGGTTCCTTTTGTAGTTGATTTCCAATTTTATTCTACTGTGG TCTGAGAGAGTGCTTGATATAATTTCAATTTTTAAAAATTTATTGAGGCTTGTTTTGTGGCATATCATATGGCCTAT CTTGGAGAAAGTTCCATGTGCTGATGAATAGAATGTGTATTCTGCAGTTGTTGGGTAGAATGTCCTGTAAATATCTG TTAAGTCCATTTGTTCTTTAAATCCATTGTTTCTTTGTAGACTGTCTTGATGACCTGCCTAGTGCAGTCAGTGGAGT ATTGAAGTCCCCCACTATTATTATGTTGCTGTCTAGTAGTAATTGTTTTATAAATTTGGGATCTCCAGTATTAGATG CATATATATTAAGAATTGTAATATTCTCCCATTGGACAAGGGCTTTTATCATTATATGATGTCCCTCTTTGTCTTTT TTAACTGCTGTTTCTTTAAAGTTTGTTTTGTCTGACATAAGAATAGCTGCTTTGGCTCGCTTTTGGTGTCCATTTGT GTGGAATGTCATTTTCCACCCCTTTACCTTAAGTTTATGTGAGTCCTTATGTGTTAGGTGAGTCTCCTGAAGGCGGC AGATAACTGGTTGGTGAATTCTATTCATTCTGCAATTCTGTATCTTTTAAGTGGAGCATTTAGTCCATTTACATTCA ACATCAGTATTGAGGTGTGAGGTGACTATTCCATTCTTCGTGGTATTTGTTGCCTGTGTATCTTTTTATCTGTATTT TTGTTGTATATGTCCTATGGGATTTATGCTTTAAAGAGGTTCTGTTTTGATGTGCTTCCAGGGTTTATTTCAAGATT TAGAGCTCCTTTTATCATTCTTGTAGTGTTGGCTTGGTAGTGCCGAATTCTCTCAGCATTTGTTTTTCTGAAAAACA CTGTGTATTTTCTTCATTTGTGAAGCTTAGTTTCACTGGATATAAAATTCTTGGCTGATAATTGTTTTGTTTAAGAA GGCTGAAGATAGGGCCATATTCACTTCTAGCTTTTACGGTTTCTGCTGAGAAATCTGCTGTTAATCTGATAGGTTTT CTTTCATAGGTTACCTGGTAGTTTCACCTCACAGCTCTTAAGATTCTCTTTGTCTTTAGATAACTTTGGATACTCTG ATGACAATGTACCTAGGCAATGATATTTTTGCAATGAATTTCCCAGGTGTTTATTGAGCTTCTTTGTATTTGGATAT CTAGGTCTCTAGCAAGGAGGGGGAAGTTTTCCTTGATTATTTCCATGGACAAGTTTTCCAAACTTTTAGATTTCTCT TCTTTCTCAGGAATGCTGATTATTCTTAGGTTTGATTGTTTAACATAATCCCAGATTTCTTGGAGGCTTTGTTCATA TTTTCTTATTCTTTTTTCTTTGTCTTTGTTGGATTGGGTAATTCAAAAACTTTGTCTTCAAGCTCTGAATTTCTTCT GCTTGGATTCTATTGCTGAGACTTTCTAGAGCATTTTGCATTTCTATAAGTGCATCCATTCATCCATTGTTTCCTGA AGTTTTGAATGTTTTTTATTTATGCTATCTCTTTAACTGAAGATTTCTCCCCTCATTTCTTGTATCATATTTTTGGT TTTTTTAAAATTGGACTTCACCTTCCTCGGATGCCTCCTTGATTAGCTTAATAACTGACCTTCTGAATTATTTTTCA GGTAAATCAGGGATTTCTTCTTGGTTTGGATGCATTGCTGGTGAGCTAGTATGATTTTTTGGGGGGTGTTAAAGAAC CTTGTTTTTCATATTACCAGAGTTAGTTTTCTGGTTCCTTCTCACTTGGGTAGGCTCTGTCAGAGGGAAAGTCTAGG CCTCAAGGCTGAGACTTTTGTCCCAGCAGGTGTTCCCTTGATGTAGCACAGTCCCCCTTTTCCTAGGACGTGGGGCT TCCTGAGAGCCGAACTGTAGTGATTGTTATCTCTCTTCTGGATCTAGCCACCCATCAGGTCTACCAGACTCCAGGCT GGTACTGGGGTTTGTCTGCACAGAGTCTTGTGACGTGAACCATCTGTGGGTCTCTCAGCCATAGATACAACCACCTG CTCCAATGGAGGTGGTAGAGGATGAAATGAACTCTGTGAGGGTCCTTACTTTTGGTTGTTCAATGCACTATCTTTTT GTGCTGGTTGGCCTCCTGCCAGGAGGTGGCACTTTCTAGAAAGCATCAGCAGAGGCAGTCAGGTGGTGGTGGCTGGG GGGGCTGGGGCACTAGAACTCCCAAGAATATATGCCCTTTGTCTTCAGCTACTAGGGTGAGTAAGGAAGGACCATCA GGTGGGGGCAGGACTAGTCGTGTCTGAGCTCAGAGTCTCCTTGGGCAGGTCTTTCTGTGGCTACTGTGGGAGGATGG GGGTGTAGTTTCCAGGTCAATGGATTTATGTTCCTAGGACAATTATGGCTGCCTCTGCTGTGTCATGCAGGTCATCA GGAAAGTGGGGGAAAGCAAGCAGTCACGTGACTTGCCCAGCTCCCATGCAACTCAAAAGGTTGGTCTCACTTCCAGC GTGCACCCTCCCCCGCAACAGCTCCGAATCTGTTTCCATGCAGTCAGTGAGCAAGGCTGAGAACTTGCCCAGGCTAC CAGCTGCGAAACCAAGTAGGGCTGTCCTACTTCCCTGCCAGTGGAGTCTGCACACCAAATTCATGTCCCCCCACCAA CCCCCCCACTGCCCAGCCCCTAGATCTGGCCAGGTGGAGATTTTCTTTTTCCTGTCTCTTTTCCCAGTTCCTCTGGC AGCCCTCCCAAATGACCCCTGTGAGGCAAGGCAGAAATGGCTTCCTAGGGGACCCAGAGAGCCCACAGGGCTTTTCC CGCTGCTTCCTCTACCCCTGTATTTTGCTTGGCCCTCTAAATTGACTCAGCTCCAGGTAAGGTCAGAATCTTCTCCT GTGGTCTAGATCTTCAGGTTCCCAGTGAGGATGTGTGTTTGGGGGTAGACGGTCCCCCTTTTCCACTTCCACAGTTT GGGCACTCACAATATTTGGGGTGTTTCCCGGGTCCTACATGAGCAATCTGCTTCTTTCAGAGGGTGTGTGCGTTCTC TCAGCTTTCTTGAATTTATTTCTGCAGGTGGTTCTGCAAAAAAAATTCCTGATGGGAGACTTCACATGCTGCTCTGT GCATCCGAGTGGGAGCTGCAATGTACTTCTGCTGCCACCCATCTGCCATCACCCTCTAATTTGTCGGTAATATGCAT TTTTAATCAATCTTTTTTTCTCTCTCTCTCTTTTCTTCTCCCCCAAAACTATACTGCCCTTTGATATCAAGGAATCA AGGCCGTGATGTTGAGGGGTGGGCAGTGGATACACTCTTTACCCCTTAGGGAGCATATCTAGATTTAGATATTGCCA ATTCAAGATAACTTAATTGAAAGCAAATTCATAATGAATACACACACACACACACACATCTGCATGACAAGATTTTT AATAGTTGAAAGAATAACTAATAATTGTCCACAGGCAATAAGGGCTTTTTAAGCAAAACAGTTGTGATAAAACAGGT CATTCTTAGAATAGTAATCCAGCCAATAGTACAGGTTGCTTAGAGATTATGACATTACCAGAGTTAAAATTCAATAA TGGCTTCTCACTCCCTACCACTGAGGACAAGTTTATGTCCTTAGGTTTATGCTTCCCTGAAACAATACCACCTGCTA TTCTCCACTTTACATATCAACGGCACTGGTTCTTTATCTAACTCTCTGGCACAGCAGGAGTTTGTTTTCTTCTGCTT CAGAGCTTTGAATTTACTATTTCAGCTTCTAAACTTTATTTGCAATGCCTTCCCATGGCAGACTCCTTCTGTCATTT TGCCTCTGTTCGAAAACTTTTTCCTTAATTTCATTCTTAGTTAATAATATCTGAAATTATTTTGTTGTTTAACTTAA TTATTAATTTTATGTATGTTCTACCTAGATATAATCTTCTAGAGGATTGTTTTATTCTCTGACTTATTTAACTTAAA TGCCCACTACCTTTAAAAATTATGACATTTATTTAACAGATATTTGCTGAACAAATGTTTGAAAATACATGGGAAAG AATGCTTGAAAACACTTGAAATTGCTTGTGTAAAGAAACAGTTTTATCAGTTAGGATTTAATCAATGTCAGAAGCAA TGATATAGGAAAAATCGAGGAATAAGACAGTTATGGATAAGGAGAAATCAACAAACTCTTAAAAGATATTGCCTCAA AAGCATAAGAGGAAATAAGGGTTTATACATGACTTTTAGAACACTGCCTGGGTTTTTGGATAAATGGGGAAGTTGTT GGAAAACAGGAGGGATCCTAGATATTCCTTAGTCTGAGGAGGAGCAATTAAGATTCACTTGTTTAGAGGCTGGGAGT GGTGGCTCACGCCTGTAATCCCAGAATTTTGGGAGGCCAAGGCAGGCAGATCACCTGAGGTCAAGAGTTCAAGACCA ACCTGGCCAACATGGTGAAATCCCATCTCTACAAAAATACAAAAATTAGACAGGCATGATGGCAAGTGCCTGTAATC CCAGCTACTTGGGAGGCTGAGGAAGGAGAATTGCTTGAACCTGGAAGGCAGGAGTTGCAGTGAGCCGAGATCATACC ACTGCACTCCAGCCTGGGTGACAGAACAAGACTCTGTCTCAAAAAAAAAAAAGAGAGATTCAAAAGATTCACTTGTT TAGGCCTTAGCGGGCTTAGACACCAGTCTCTGACACATTCTTAAAGGTCAGGCTCTACAAATGGAACCCAACCAGAC TCTCAGATATGGCCAAAGATCTATACACACCCATCTCACAGATCCCCTATCTTAAAGAGACCCTAATTTGGGTTCAC CTCAGTCTCTATAATCTGTACCAGCATACCAATAAAAATCTTTCTCACCCATCCTTAGATTGAGAGAAGTCACTTAT TATTATGTGAGTAACTGGAAGATACTGATAAGTTGACAAATCTTTTTCTTTCCTTTCTTATTCAACTTTTATTTTAA CTTCCAAAGAACAAGTGCAATATGTGCAGCTTTGTTGCGCAGGTCAACATGTATCTTTCTGGTCTTTTAGCCGCCTA ACACTTTGAGCAGATATAAGCCTTACACAGGATTATGAAGTCTGAAAGGATTCCACCAATATTATTATAATTCCTAT CAACCTGATAAGTTAGGGGAAGGTAGAGCTCTCCTCCAATAAGCCAGATTTCCAGAGTTTCTGACGTCATAATCTAC CAAGGTCATGGATCGAGTTCAGAGAAAAAACAAAAGCAAAACCAAACCTACCAAAAAATAAAAATCCCAAAGAAAAA ATAAAGAAAAAAACAGCATGAATACTTCCTGCCATGTTAAGTGGCCAATATGTCAGAAACAGCACTGAGTTACAGAT AAAGATGTCTAAACTACAGTGACATCCCAGCTGTCACAGTGTGTGGACTATTAGTCAATAAAACAGTCCCTGCCTCT TAAGAGTTGTTTTCCATGCAAATACATGTCTTATGTCTTAGAATAAGATTCCCTAAGAAGTGAACCTAGCATTTATA CAAGATAATTAATTCTAATCCATAGTATCTGGTAAAGAGCATTCTACCATCATCTTTACCGAGCATAGAAGAGCTAC ACCAAAACCCTGGGTCATCAGCCAGCACATACACTTATCCAGTGATAAATACACATCATCGGGTGCCTACATACATA CCTGAATATAAAAAAAATACTTTTGCTGAGATGAAACAGGCGTGATTTATTTCAAATAGGTACGGATAAGTAGATAT TGAAGTAAGGATTCAGTCTTATATTATATTACATAACATTAATCTATTCCTGCACTGAAACTGTTGCTTTATAGGAT TTTTCACTACACTAATGAGAACTTAAGAGATAATGGCCTAAAACCACAGAGAGTATATTCAAGAATAAGTATAGCAC TTCTTATTTGGAAACCAATGCTTACTAAATGAGACTAAGACGTGTCCCATCAAAAATCCTGGACCTATGCCTAAAAC ACATTTCACAATCCCTGAACTTTTCAAAAATTGGTACATGCTTTAACTTTAAACTACAGGCCTCACTGGAGCTACAG ACAAGAAGGTGAAAAACGGCTGACAAAAGAAGTCCTGGTATCTTCTATGGTGGGAGAAGAAAACTAGCTAAAGGGAA GAATAAATTAGAGAAAAATTGGAATGACTGAATCGGAACAAGGCAAAGGCTATAAAAAAAATTAAGCAGCAGTATCC TCTTGGGGGCCCCTTCCCCACACTATCTCAATGCAAATATCTGTCTGAAACGGTTCCTGGCTAAACTCCACCCATGG GTTGGCCAGCCTTGCCTTGACCAATAGCCTTGACAAGGCAAACTTGACCAATAGTCTTAGAGTATCCAGTGAGGCCA GGGGCCGGCGGCTGGCTAGGGATGAAGAATAAAAGGAAGCACCCTTCAGCAGTTCCACACACTCGCTTCTGGAACGT CTGAGGTTATCAATAAGCTCCTAGTCCAGACGCCATGGGTCATTTCACAGAGGAGGACAAGGCTACTATCACAAGCC TGTGGGGCAAGGTGAATGTGGAAGATGCTGGAGGAGAAACCCTGGGAAGGTAGGCTCTGGTGACCAGGACAAGGGAG GGAAGGAAGGACCCTGTGCCTGGCAAAAGTCCAGGTCGCTTCTCAGGATTTGTGGCACCTTCTGACTGTCAAACTGT TCTTGTCAATCTCACAGGCTCCTGGTTGTCTACCCATGGACCCAGAGGTTCTTTGACAGCTTTGGCAACCTGTCCTC TGCCTCTGCCATCATGGGCAACCCCAAAGTCAAGGCACATGGCAAGAAGGTGCTGACTTCCTTGGGAGATGCCATAA AGCACCTGGATGATCTCAAGGGCACCTTTGCCCAGCTGAGTGAACTGCACTGTGACAAGCTGCATGTGGATCCTGAG AACTTCAAGGTGAGTCCAGGAGATGTTTCAGCACTGTTGCCTTTAGTCTCGAGGCAACTTAGACAACTGAGTATTGA TCTGAGCACAGCAGGGTGTGAGCTGTTTGAAGATACTGGGGTTGGGAGTGAAGAAACTGCAGAGGACTAACTGGGCT GAGACCCAGTGGCAATGTTTTAGGGCCTAAGGAGTGCCTCTGAAAATCTAGATGGACAACTTTGACTTTGAGAAAAG AGAGGTGGAAATGAGGAAAATGACTTTTCTTTATTAGATTTCGGTAGAAAGAACTTTCACCTTTCCCCTATTTTTGT TATTCGTTTTAAAACATCTATCTGGAGGCAGGACAAGTATGGTCGTTAAAAAGATGCAGGCAGAAGGCATATATTGG CTCAGTCAAAGTGGGGAACTTTGGTGGCCAAACATACATTGCTAAGGCTATTCCTATATCAGCTGGACACATATAAA ATGCTGCTAATGCTTCATTACAAACTTATATCCTTTAATTCCAGATGGGGGCAAAGTATGTCCAGGGGTGAGGAACA ATTGAAACATTTGGGCTGGAGTAGATTTTGAAAGTCAGCTCTGTGTGTGTGTGTGTGTGTGTGCGCGCGTGTGTTTG TGTGTGTGTGAGAGCGTGTGTTTCTTTTAACGTTTTCAGCCTACAGCATACAGGGTTCATGGTGGCAAGAAGATAAC AAGATTTAAATTATGGCCAGTGACTAGTGCTGCAAGAAGAACAACTACCTGCATTTAATGGGAAAGCAAAATCTCAG GCTTTGAGGGAAGTTAACATAGGCTTGATTCTGGGTGGAAGCTTGGTGTGTAGTTATCTGGAGGCCAGGCTGGAGCT CTCAGCTCACTATGGGTTCATCTTTATTGTCTCCTTTCATCTCAACAGCTCCTGGGAAATGTGCTGGTGACCGTTTT GGCAATCCATTTCGGCAAAGAATTCACCCCTGAGGTGCAGGCTTCCTGGCAGAAGATGGTGACTGGAGTGGCCAGTG CCCTGTCCTCCAGATACCACTGAGCTCACTGCCCATGATGCAGAGCTTTCAAGGATAGGCTTTATTCTGCAAGCAAT ACAAATAATAAATCTATTCTGCTAAGAGATCACACATGGTTGTCTTCAGTTCTTTTTTTTATGTCTTTTTAAATATA TGAGCCACAAAGGGTTTTATGTTGAGGGATGTGTTTATGTGTATTTATACATGGCTATGTGTGTTTGTGTCATGTGC ACACTCCACACTTTTTTGTTTACGTTAGATGTGGGTTTTGATGAGCAAATAAAAGAACTAGGCAATAAAGAAACTTA TACATGGGAGCGTCTGCAAGTGGGAGTAAAAGGTGCAGGAGAAATCTGGTTGGAAGAAAGACCTCTATAGGACAGGA CTCCTCAGAAACAGATGTTTTGGAAGAGATGGGGAAAGGTTCAGTGAAGGGGGCTGAACCCCCTTCCCTGGATTGCA GCACAGCAGCGAGGAAGGGGCTCAACGAAGAAAAAGTGTTCCAAGCTTTAGGAAGTCAAGGTTTAGGCAGGGATAGC CATTCTATTTTATTAGGGGCAATACTATTTCCAACGGCATCTGGCTTTTCTCAGCCCTTGTGAGGCTCTACGGGGAG GTTGAGGTGTTAGAGATCAGAGCAGGAAACAGGTTTTTCTTTCCACGGTAACTACAATGAAGTGATCCTTACTTTAC TAAGGAACTTTTTCATTTTAAGTGTTGACGCATGCCTAAAGAGGTGAAATTAATCCCATACCCTTAAGTCTACAGAC TGGTCACAGCATTTCAAGGAGGAGACCTCATTGTAAGCTTCTAGGGAGGTGGGGACCTAGGTGAAGGAAATGAGCCA GCAGAAGCTCACAAGTCAGCATCAGCGTGTCATGTCTCAGCAGCAGAACAGCACGGTCAGATGAAAATATAGTGTGA AGAATTTGTATAACATTAATTGAGAAGGCAGATTCACTGGAGTTCTTATATAATTGAAAGTTAATGCACGTTAATAA GCAAGAGTTTAGTTTAATGTGATGGTGTTATGAACTTAACGCTTGTGTCTCCAGAAAATTCACATGCTGAATCCCCA ACTCCCAATTGGCTCCATTTGTGGGGGAGGCTTTGGAAAAGTAATCAGGTTTAGAGGAGCTCATGAGAGCAGATCCC CATCATAGAATTATTTTCCTCATCAGAAGCAGAGAGATTAGCCATTTCTCTTCCTTCTGGTGAGGACACAGTGGGAA GTCAGCCACCTGCAACCCAGGAAGAGAGCCCTGACCAGGAACCAGCAGAAAAGTGAGAAAAAATCCTGTTGTTGAAG TCACCCAGTCTATGCTATTTTGTTATAGCACCTTGCACTAAGTAAGGCAGATGAAGAAAGAGAAAAAAATAAGCTTC GGTGTTCAGTGGATTAGAAACCATGTTTATCTCAGGTTTACAAATCTCCACTTGTCCTCTGTGTTTCAGAATAAAAT ACCAACTCTACTACTCTCATCTGTAAGATGCAAATAGTAAGCCTGATCCCTTCTGTCTAACTTCGAATTCTATTTTT TCTTCAACGTACTTTAGGCTTGTAATGTGTTTATATACAGTGAAATGTCAAGTTCTTTCTTTATATTTCTTTCTTTC TTTTTTTTCCTCAGCCTCAGAGTTTTCCACATGCCCTTCCTACCTTCAGGAACTTCTTTCTCCAAACGTCTTCTGCC TGGCCTCCATTCAAATCATAAAGGACCCACTTCAAATGCCATCACTCACTACCATTTCACAATTCGCACTTTCTTTC TTTGTCCTTTTTTTTTTTAGTAAAACAAGTTTATAAAAAATTGAAGGAATAAATGAATGGCTACTTCATAGGCAGAG TAGACACAAGGGCTACTGGTTGCCGATTTTTATTGTTATTTTTCAATAGTATGCTAAACAAGGGGTAGATTATTTAT GCTGCCCATTTTTAGACCATAAAAGATAACTTCCTGATGTTGCCATGGCATTTTTTTTCCTTTTAATTTTATTTCAT TTCATTTTAATTTCGAAGGTACATGTGCAGGATGTGCAGGCTTGTTACATGGGTAAATGTGTGTCTTTCTGGCCTTT TAGCCATCTGTATCAATGAGCAGATATAAGCTTTACACAGGATCATGAAGGATGAAAGAATTTCACCAATATTATAA TAATTTCAATCAACCTGATAGCTTAGGGGATAAACTAATTTGAAGATACAGCTTGCCTCCGATAAGCCAGAATTCCA GAGCTTCTGGCATTATAATCTAGCAAGGTTAGAGATCATGGATCACTTTCAGAGAAAAACAAAAACAAACTAACCAA AAGCAAAACAGAACCAAAAAACCTCCATAAATACTTCCTACCCAGTTAATGGTCCAATATGTCAGAAACAGCACTGT GTTAGAAATAAAGCTGTCTAAAGTACACTAATATTCGAGTTATAATAGTGTGTGGACTATTAGTCAATAAAAACAAC CCTTGCCTCTTTAGAGTTGTTTTCCATGTACACGCACATCTTATGTCTTAGAGTAAGATTCCCTGAGAAGTGAACCT AGCATTTATACAAGATAATTAATTCTAATCCACAGTACCTGCCAAAGAACATTCTACCATCATCTTTACTGAGCATA GAAGAGCTACGCCAAAACCCTGGGTCATCAGCCAGCACACACACTTATCCAGTGGTAAATACACATCATCTGGTGTA TACATACATACCTGAATATGGAATCAAATATTTTTCTAAGATGAAACAGTCATGATTTATTTCAAATAGGTACGGAT AAGTAGATATTGAGGTAAGCATTAGGTCTTATATTATGTAACACTAATCTATTACTGCGCTGAAACTGTGGTCTTTA TGAAAATTGTTTTCACTACACTATTGAGAAATTAAGAGATAATGGCAAAAGTCACAAAGAGTATATTCAAAAAGAAG TATAGCACTTTTTCCTTAGAAACCACTGCTAACTGAAAGAGACTAAGATTTGTCCCGTCAAAAATCCTGGACCTATG CCTAAAACACATTTCACAATCCCTGAACTTTTCAAAAATTGGTACATGCTTTAGCTTTAAACTACAGGCCTCACTGG AGCTACAGACAAGAAGGTAAAAAACGGCTGACAAAAGAAGTCCTGGTATCCTCTATGATGGGAGAAGGAAACTAGCT AAAGGGAAGAATAAATTAGAGAAAAACTGGAATGACTGAATCGGAACAAGGCAAAGGCTATAAAAAAAATTAAGCAG CAGTATCCTCTTGGGGGCCCCTTCCCCACACTATCTCAATGCAAATATCTGTCTGAAACGGTCCCTGGCTAAACTCC ACCCATGGGTTGGCCAGCCTTGCCTTGACCAATAGCCTTGACAAGGCAAACTTGACCAATAGTCTTAGAGTATCCAG TGAGGCCAGGGGCCGGCGGCTGGCTAGGGATGAAGAATAAAAGGAAGCACCCTTCAGCAGTTCCACACACTCGCTTC TGGAACGTCTGAGATTATCAATAAGCTCCTAGTCCAGACGCCATGGGTCATTTCACAGAGGAGGACAAGGCTACTAT CACAAGCCTGTGGGGCAAGGTGAATGTGGAAGATGCTGGAGGAGAAACCCTGGGAAGGTAGGCTCTGGTGACCAGGA CAAGGGAGGGAAGGAAGGACCCTGTGCCTGGCAAAAGTCCAGGTCGCTTCTCAGGATTTGTGGCACCTTCTGACTGT CAAACTGTTCTTGTCAATCTCACAGGCTCCTGGTTGTCTACCCATGGACCCAGAGGTTCTTTGACAGCTTTGGCAAC CTGTCCTCTGCCTCTGCCATCATGGGCAACCCCAAAGTCAAGGCACATGGCAAGAAGGTGCTGACTTCCTTGGGAGA TGCCATAAAGCACCTGGATGATCTCAAGGGCACCTTTGCCCAGCTGAGTGAACTGCACTGTGACAAGCTGCATGTGG ATCCTGAGAACTTCAAGGTGAGTCCAGGAGATGTTTCAGCACTGTTGCCTTTAGTCTCGAGGCAACTTAGACAACTG AGTATTGATCTGAGCACAGCAGGGTGTGAGCTGTTTGAAGATACTGGGGTTGGGAGTGAAGAAACTGCAGAGGACTA ACTGGGCTGAGACCCAGTGGCAATGTTTTAGGGCCTAAGGAGTGCCTCTGAAAATCTAGATGGACAACTTTGACTTT GAGAAAAGAGAGGTGGAAATGAGGAAAATGACTTTTCTTTATTAGATTTCGGTAGAAAGAACTTTCACCTTTCCCCT ATTTTTGTTATTCGTTTTAAAACATCTATCTGGAGGCAGGACAAGTATGGTCGTTAAAAAGATGCAGGCAGAAGGCA TATATTGGCTCAGTCAAAGTGGGGAACTTTGGTGGCCAAACATACATTGCTAAGGCTATTCCTATATCAGCTGGACA CATATAAAATGCTGCTAATGCTTCATTACAAACTTATATCCTTTAATTCCAGATGGGGGCAAAGTATGTCCAGGGGT GAGGAACAATTGAAACATTTGGGCTGGAGTAGATTTTGAAAGTCAGCTCTGTGTGTGTGTGTGTGTGTGTGTGTGTC AGCGTGTGTTTCTTTTAACGTCTTCAGCCTACAACATACAGGGTTCATGGTGGGAAGAAGATAGCAAGATTTAAATT ATGGCCAGTGACTAGTGCTTGAAGGGGAACAACTACCTGCATTTAATGGGAAGGCAAAATCTCAGGCTTTGAGGGAA GTTAACATAGGCTTGATTCTGGGTGGAAGCTGGGTGTGTAGTTATCTGGAGGCCAGGCTGGAGCTCTCAGCTCACTA TGGGTTCATCTTTATTGTCTCCTTTCATCTCAACAGCTCCTGGGAAATGTGCTGGTGACCGTTTTGGCAATCCATTT CGGCAAAGAATTCACCCCTGAGGTGCAGGCTTCCTGGCAGAAGATGGTGACTGCAGTGGCCAGTGCCCTGTCCTCCA GATACCACTGAGCCTCTTGCCCATGATTCAGAGCTTTCAAGGATAGGCTTTATTCTGCAAGCAATACAAATAATAAA TCTATTCTGCTGAGAGATCACACATGATTTTCTTCAGCTCTTTTTTTTACATCTTTTTAAATATATGAGCCACAAAG GGTTTATATTGAGGGAAGTGTGTATGTGTATTTCTGCATGCCTGTTTGTGTTTGTGGTGTGTGCATGCTCCTCATTT ATTTTTATATGAGATGTGCATTTTGATGAGCAAATAAAAGCAGTAAAGACACTTGTACACGGGAGTTCTGCAAGTGG GAGTAAATGGTGTTGGAGAAATCCGGTGGGAAGAAAGACCTCTATAGGACAGGACTTCTCAGAAACAGATGTTTTGG AAGAGATGGGAAAAGGTTCAGTGAAGACCTGGGGGCTGGATTGATTGCAGCTGAGTAGCAAGGATGGTTCTTAATGA AGGGAAAGTGTTCCAAGCTTTAGGAATTCAAGGTTTAGTCAGGTGTAGCAATTCTATTTTATTAGGAGGAATACTAT TTCTAATGGCACTTAGCTTTTCACAGCCCTTGTGGATGCCTAAGAAAGTGAAATTAATCCCATGCCCTCAAGTGTGC AGATTGGTCACAGCATTTCAAGGGAGAGACCTCATTGTAAGACTCTGGGGGAGGTGGGGACTTAGGTGTAAGAAATG AATCAGCAGAGGCTCACAAGTCAGCATGAGCATGTTATGTCTGAGAAACAGACCAGCACTGTGAGATCAAAATGTAG TGGGAAGAATTTGTACAACATTAATTGGAAGGTTTACTTAATGGAATTTTTGTATAGTTGGATGTTAGTGCATCTCT ATAAGTAAGAGTTTAATATGATGGTGTTACGGACCTGGTGTTTGTGTCTCCTCAAAATTCACATGCTGAATCCCCAA CTCCCAACTGACCTTATCTGTGGGGGAGGCTTTTGAAAAGTAATTAGGTTTAGCTGAGCTCATAAGAGCAGATCCCC ATCATAAAATTATTTTCCTTATCAGAAGCAGAGAGACAAGCCATTTCTCTTTCCTCCCGGTGAGGACACAGTGAGAA GTCCGCCATCTGCAATCCAGGAAGAGAACCCTGACCACGAGTCAGCCTTCAGAAATGTGAGAAAAAACTCTGTTGTT GAAGCCACCCAGTCTTTTGTATTTTGTTATAGCACCTTACACTGAGTAAGGCAGATGAAGAAGGAGAAAAAAATAAG CTTGGGTTTTGAGTGAACTACAGACCATGTTATCTCAGGTTTGCAAAGCTCCCCTCGTCCCCTATGTTTCAGCATAA AATACCTACTCTACTACTCTCATCTATAAGACCCAAATAATAAGCCTGCGCCCTTCTCTCTAACTTTGATTTCTCCT ATTTTTACTTCAACATGCTTTACTCTAGCCTTGTAATGTCTTTACATACAGTGAAATGTAAAGTTCTTTATTCTTTT TTTCTTTCTTTCTTTTTTCTCCTCAGCCTCAGAATTTGGCACATGCCCTTCCTTCTTTCAGGAACTTCTCCAACATC TCTGCCTGGCTCCATCATATCATAAAGGTCCCACTTCAAATGCAGTCACTACCGTTTCAGGATATGCACTTTCTTTC TTTTTTGTTTTTTGTTTTTTTTAAGTCAAAGCAAATTTCTTGAGAGAGTAAAGAAATAAACGAATGACTACTGCATA GGCAGAGCAGCCCCGAGGGCCGCTGGTTGTTCCTTTTATGGTTATTTCTTGATGATATGTTAAACAAGTTTTGGATT ATTTATGCCTTCTCTTTTTAGGCCATATAGGGTAACTTTCTGACATTGCCATGGCATGTTTCTTTTAATTTAATTTA CTGTTACCTTAAATTCAGGGGTACACGTACAGGATATGCAGGTTTGTTTTATAGGTAAAAGTGTGCCATGGTTTTAA TGGGTTTTTTTTTTCTTGTAAAGTTGTTTAAGTTTCTTGTTTACTCTGGATATTGGCCTTTGTCAGAAGAATAGATT GGAAAATCTTTTTCCCATTCTGTAGATTGTCTTTCGCTCTGATGGTAGTTTCTTTTGCTGAGCAGGAGCTCTTTAGT TTAATTAGATTCCATTGGTCAATTTTTGCTTTTGCTGCAATTGCTTTTCACGCTTTCATCATGAAATCTGTGCCCGT GTTTATATCATGAATAGTATTGCCTTGATTTTTTTCTAGGCTTTTTATAGTTTGGGGTTTTTCATTTAAGTCTCTAA TCCATCCGGAGTTAATTTTGGATAAGGTATAAGGAAGGAGTCCAGTTTCATTTTTCAGCATATGGCTAGCCAGTTCT CCCCCATCATTTATTAAATTGAAAATCCTTTCCCCATTGCTTGCTTTTGTCAGGTTTCTAAAAGACAGATGGTTGTA GGTACAATATGCAGTTTCTTCAAGTCATATAATACCATCTGAAATCTCTTATTAATTCATTTCTTTTAGTATGTATG CTGGTCTCCTCTGCTCACTATAGTGAGGGCACCATTAGCCAGAGAATCTGTCTGTCTAGTTCATGTAAGATTCTCAG AATTAAGAAAAATGGATGGCATATGAATGAAACTTCATGGATGACATATGGAATCTAATGTGTATTTGTTGAATTAA TGCATAAGATGCAACAAGGGAAAGGTTGACAACTGCAGTGATAACCTGGTATTGATGATATAAGAGTCTATAGATCA CAGTAGAAGCAATAATCATGGAAAACAATTGGAAATGGGGAACAGCCACAAACAAGAAAGAATCAATACTACCAGGA AAGTGACTGCAGGTCACTTTTCCTGGAGCGGGTGAGAGAAAAGTGGAAGTTGCAGTAACTGCCGAATTCCTGGTTGG CTGATGGAAAGATGGGGCAACTGTTCACTGGTACGCAGGGTTTTAGATGTATGTACCTAAGGATATGAGGTATGGCA ATGAACAGAAATTCTTTTGGGAATGAGTTTTAGGGCCATTAAAGGACATGACCTGAAGTTTCCTCTGAGGCCAGTCC CCACAACTCAATATAAATGTGTTTCCTGCATATAGTCAAAGTTGCCACTTCTTTTTCTTCATATCATCGATCTCTGC TCTTAAAGATAATCTTGGTTTTGCCTCAAACTGTTTGTCACTACAAACTTTCCCCATGTTCCTAAGTAAAACAGGTA ACTGCCTCTCAACTATATCAAGTAGACTAAAATATTGTGTCTCTAATATCAGAAATTCAGCTTTAATATATTGGGTT TAACTCTTTGAAATTTAGAGTCTCCTTGAAATACACATGGGGGTGATTTCCTAAACTTTATTTCTTGTAAGGATTTA TCTCAGGGGTAACACACAAACCAGCATCCTGAACCTCTAAGTATGAGGACAGTAAGCCTTAAGAATATAAAATAAAC TGTTCTTCTCTCTGCCGGTGGAAGTGTGCCCTGTCTATTCCTGAAATTGCTTGTTTGAGACGCATGAGACGTGCAGC ACATGAGACACGTGCAGCAGCCTGTGGAATATTGTCAGTGAAGAATGTCTTTGCCTGATTAGATATAAAGACAAGTT AAACACAGCATTAGACTATAGATCAAGCCTGTGCCAGACACAAATGACCTAATGCCCAGCACGGGCCACGGAATCTC CTATCCTCTTGCTTGAACAGAGCAGCACACTTCTCCCCCAACACTATTAGATGTTCTGGCATAATTTTGTAGATATG TAGGATTTGACATGGACTATTGTTCAATGATTCAGAGGAAATCTCCTTTGTTCAGATAAGTACACTGACTACTAAAT GGATTAAAAAACACAGTAATAAAACCCAGTTTTCCCCTTACTTCCCTAGTTTGTTTCTTATTCTGCTTTCTTCCAAG TTGATGCTGGATAGAGGTGTTTATTTCTATTCTAAAAAGTGATGAAATTGGCCGGGCGCGGTGGCTCACACCTGTAA TCCCAGCACTTTGGGAGGCTGAGGTGGGCGGATCACGAGGTCAGGAGATCAAGACCATCCTGGCTAACATGGTGAAA CCCCATCTCTACTAAAAATACAAAAAATTAGCCAGAGACGGTGGCGGGTGCCTGTAGTCCCAGCTACTCGGGAGGCT GAGGCAGGAGAATGGCGTGAACCTGGGAGGCAGAGCTGCAGTGAGCAGAGATCGCGCCACTGCACACTCCAGCCTGG GTGACAAAGCGAGACTCCATCTCAAAAAAAAAAAAAAAAAAAAAAAGAAAGAAAGAAAGAAAAAAAAAGTGATGAAA TTGTGTATTCAATGTAGTCTCAAGAGAATTGAAAACCAAGAAAGGCTGTGGCTTCTTCCACATAAAGCCTGGATGAA TAACAGGATAACACGTTGTTACATTGTCACAACTCCTGATCCAGGAATTGATGGCTAAGATATTCGTAATTCTTATC CTTTTCAGTTGTAACTTATTCCTATTTGTCAGCATTCAGGTTATTAGCGGCTGCTGGCGAAGTCCTTGAGAAATAAA CTGCACACTGGATGGTGGGGGTAGTGTAGGAAAATGGAGGGGAAGGAAGTAAAGTTTCAAATTAAGCCTGAACAGCA AAGTTCCCCTGAGAAGGCCACCTGGATTCTATCAGAAACTCGAATGTCCATCTTGCAAAACTTCCTTGCCCAAACCC CACCCCTGGAGTCACAACCCACCCTTGACCAATAGATTCATTTCACTGAGGGAGGCAAAGGGCTGGTCAATAGATTC ATTTCACTGGGAGAGGCAAAGGGCTGGGGGCCAGAGAGGAGAAGTAAAAAGCCACACATGAAGCAGCAATGCAGGCA TGCTTCTGGCTCATCTGTGATCACCAGGAAACTCCCAGATCTGACACTGTAGTGCATTTCACTGCTGACAAGAAGGC TGCTGCCACCAGCCTGTGAAGCAAGGTTAAGGTGAGAAGGCTGGAGGTGAGATTCTGGGCAGGTAGGTACTGGAAGC CGGGACAAGGTGCAGAAAGGCAGAAAGTGTTTCTGAAAGAGGGATTAGCCCGTTGTCTTACATAGTCTGACTTTGCA CCTGCTCTGTGATTATGACTATCCCACAGTCTCCTGGTTGTCTACCCATGGACCTAGAGGTACTTTGAAAGTTTTGG ATATCTGGGCTCTGACTGTGCAATAATGGGCAACCCCAAAGTCAAGGCACATGGCAAGAAGGTGCTGATCTCCTTCG GAAAAGCTGTTATGCTCACGGATGACCTCAAAGGCACCTTTGCTACACTGAGTGACCTGCACTGTAACAAGCTGCAC GTGGACCCTGAGAACTTCCTGGTGAGTAGTAAGTACACTCACGCTTTCTTCTTTACCCTTAGATATTTGCACTATGG GTACTTTTGAAAGCAGAGGTGGCTTTCTCTTGTGTTATGAGTCAGCTATGGGATATGATATTTCAGCAGTGGGATTT TGAGAGTTATGTTGCTGTAAATAACATAACTAAAATTTGGTAGAGCAAGGACTATGAATAATGGAAGGCCACTTACC ATTTGATAGCTCTGAAAAACACATCTTATAAAAAATTCTGGCCAAAATCAAACTGAGTGTTTTGGATGAGGGAACAG AAGTTGAGATAGAGAAAATAACATCTTTCCTTTGGTCAGCGAAATTTTCTATAAAAATTAATAGTCACTTTTCTGCA TAGTCCTGGAGGTTAGAAAAAGATCAACTGAACAAAGTAGTGGGAAGCTGTTAAAAGAGGATTGTTTCCCTCCGAAT GATGATGGTATACTTTTGTACGCATGGTACAGGATTCTTTGTTATGAGTGTTTGGGAAAATTGTATGTATGTATGTA TGTATGTGATGACTGGGGACTTATCCTATCCATTACTGTTCCTTGAAGTACTATTATCCTACTTTTTAAAAGGACGA AGTCTCTAAAAAAAAAATGAAACAATCACAATATGTTGGGGTAGTGAGTTGGCATAGCAAGTAAGAGAAGGATAGGA CACAATGGGAGGTGCAGGGCTGCCAGTCATATTGAAGCTGATATCTAGCCCATAATGGTGAGAGTTGCTCAAACTCT GGTCAAAAAGGATGTAAGTGTTATATCTATTTACTGCAAGTCCAGCTTGAGGCCTTCTATTCACTATGTACCATTTT CTTTTTTATCTTCACTCCCTCCCCAGCTCTTAGGCAACGTGATATTGATTGTTTTGGCAACCCACTTCAGCGAGGAT TTTACCCTACAGATACAGGCTTCTTGGCAGTAACTAACAAATGCTGTGGTTAATGCTGTAGCCCACAAGACCACTGA GTTCCCTGTCCACTATGTTTGTACCTATGTCCCAAAATCTCATCTCCTTTAGATGGGGGAGGTTGGGGAGAAGAGCA GTATCCTGCCTGCTGATTCAGTTCCTGCATGATAAAAATAGAATAAAGAAATATGCTCTCTAAGAAATATCATTGTA CTCTTTTTCTGTCTTTATATTTTACCCTGATTCAGCCAAAAGGACGCACTATTTCTGATGGAAATGAGAATGTTGGA GAATGGGAGTTTAAGGACAGAGAAGATACTTTCTTGCAATCCTGCAAGAAAAGAGAGAACTCGTGGGTGGATTTAGT GGGGTAGTTACTCCTAGGAAGGGGAAATCGTCTCTAGAATAAGACAATGTTTTTACAGAAAGGGAGGTCAATGGAGG TACTCTTTGGAGGTGTAAGAGGATTGTTGGTAGTGTGTAGAGGTATGTTAGGACTCAAATTAGAAGTTCTGTATAGG CTATTATTTGTATGAAACTCAGGATATAGCTCATTTGGTGACTGCAGTTCACTTCTACTTATTTTAAACAACATATT TTTTATGATTTATAATGAAGTGGGGATGGGGCTTCCTAGAGACCAATCAAGGGCCAAACCTTGAACTTTCTCTTAAC GTCTTCAATGGTATTAATAGAGAATTATCTCTAAGGCATGTGAACTGGCTGTCTTGGTTTTCATCTGTACTTCATCT GCTACCTCTGTGACCTGAAACATATTTATAATTCCATTAAGCTGTGCATATGATAGATTTATCATATGTATTTTCCT TAAAGGATTTTTGTAAGAACTAATTGAATTGATACCTGTAAAGTCTTTATCACACTACCCAATAAATAATAAATCTC TTTGTTCAGCTCTCTGTTTCTATAAATATGTACAAGTTTTATTGTTTTTAGTGGTAGTGATTTTATTCTCTTTCTAT ATATATACACACACATGTGTGCATTCATAAATATATACAATTTTTATGAATAAAAAATTATTAGCAATCAATATTGA AAACCACTGATTTTTGTTTATGTGAGCAAACAGCAGATTAAAAGGCTGAGATTTAGGAAACAGCACGTTAAGTCAAG TTGATAGAGGAGAATATGGACATTTAAAAGAGGCAGGATGATATAAAATTAGGGAAACTGGATGCAGAGACCAGATG AAGTAAGAAAAATAGCTATCGTTTTGAGCAAAAATCACTGAAGTTTCTTGCATATGAGAGTGACATAATAAATAGGG AAACGTAGAAAATTGATTCACATGTATATATATATATAGAACTGATTAGACAAAGTCTAACTTGGGTATAGTCAGAG GAGCTTGCTGTAATTATATTGAGGTGATGGATAAAGAACTGAAGTTGATGGAAACAATGAAGTTAAGAAAAAAAATC GAGTAAGAGACCATTGTGGCAGTGATTGCACAGAACTGGAAAACATTGTGAAACAGAGAGTCAGAGATGACAGCTAA AATCCCTGTCTGTGAATGAAAAGAAGGAAATTTATTGACAGAACAGCAAATGCCTACAAGCCCCCTGTTTGGATCTG GCAATGAACGTAGCCATTCTGTGGCAATCACTTCAAACTCCTGTACCCAAGACCCTTAGGAAGTATGTAGCACCCTC AAACCTAAAACCTCAAAGAAAGAGGTTTTAGAAGATATAATACCCTTTCTTCTCCAGTTTCATTAATCCCAAAACCT CTTTCTCAAAGTATTTCCTCTATGTGTCCACCCCAAAGAGCTCACCTCACCATATCTCTTGAGTGGGAGCACATAGA TAGGCGGTGCTACCATCTAACAGCTTCTGAAATTCCTTTGTCATATTTTTGAGTCCCCACTAATAACCCACAAAGCA GAATAAATACCAGTTGCTCATGTACAATAATCACTCAACTGCTGTCTTGTAGCATACATTAATTAAGCACATTCTTT GAATAATTACTGTGTCCAAACAATCACACTTTAAAATCTCACACTTGTGCTATCCCTTGCCCTTCTGAATGTCACTC TGTATTTTAAATGAAGAGATGAGGGTTGAATTTCCTGTGTTACTTATTGTTCATTTCTCGATGAGGAGTTTTCACAT TCACCTTTACTGGAAAACACATAAGTACACATCTTACAGGAAAAATATACCAAACTGACATGTAGCATGAATGCTTG TGCATGTAGTCATATAAAATCTTGTAGCAATGTAAACATTCTCTGATATACACATACAGATGTGTCTATATGTCTAC ACAATTTCTTATGCTCCATGAACAAACATTCCATGCACACATAAGAACACACACTGTTACAGATGCATACTTGAGTG CATTGACAAAATTACCCCAGTCAATCTAGAGAATTTGGATTTCTGCATTTGACTCTGTTAGCTTTGTACATGCTGTT CATTTACTCTGGGTGATGTCTTTCCCTCATTTTGCCTTGTCTATCTTGTACTCATACTTTAAGTCCTAACTTATATG TTATCTCAACTAAGAAGCTATTTTTTTTTAATTTTAACTGGGCTTAAAGCCCTGTCTATAAACTCTGCTACAATTAT GGGCTCTTTCTTATAATATTTAGTGTTTTTCCTACTAATGTACTTAATCTGCTCATTGTATATTCCTACCACTAAAT TTTAACCTCTTTTATGGTAGAGACATTGTCTTGTAAACTCTTATTTCCCTAGTATTTGGAGATGAAAAAAAAGATTA AATTATCCAAAATTAGATCTCTCTTTTCTACATTATGAGTATTACACTATCCATAGGGAAGTTTGTTTGAGACCTAA ACTGAGGAACCTTTGGTTCTAAAATGACTATGTGATATCTTAGTATTTATAGGTCATGAGGTTCCTTCCTCTGCCTC TGCTATAGTTTGATTAGTCAGCAAGCATGTGTCATGCATTTATTCACATCAGAATTTCATACACTAATAAGACATAG TATCAGAAGTCAGTTTATTAGTTATATCAGTTAGGGTCCATCAAGGAAAGGACAAACCATTATCAGTTACTCAACCT AGAATTAAATACAGCTCTTAATAGTTAATTATCCTTGTATTGGAAGAGCTAAAATATCAAATAAAGGACAGTGCAGA AATCTAGATGTTAGTAACATCAGAAAACCTCTTCCGCCATTAGGCCTAGAAGGGCAGAAGGAGAAAATGTTTATACC ACCAGAGTCCAGAACCAGAGCCCATAACCAGAGGTCCACTGGATTCAGTGAGCTAGTGGGTGCTCCTTGGAGAGAGC CAGAACTGTCTAATGGGGGCATCAAAGTATCAGCCATAAAAAACCATAAAAAAGACTGTCTGCTGTAGGAGATCCGT TCAGAGAGAGAGAGAGACCAGAAATAATCTTGCTTATGCTTTCCCTCAGCCAGTGTTTACCATTGCAGAATGTACAT GCGACTGAAAGGGTGAGGAAACCTGGGAAATGTCAGTTCCTCAAATACAGAGAACACTGAGGGAAGGATGAGAAATA AATGTGAAAGCAGACATGAATGGTAATTGACAGAAGGAAACTAGGATGTGTCCAGTAAATGAATAATTACAGTGTGC AGTGATTATTGCAATGATTAATGTATTGATAAGATAATATGAAAACACAGAATTCAAACAGCAGTGAACTGAGATTA GAATTGTGGAGAGCACTGGCATTTAAGAATGTCACACTTAGAATGTGTCTCTAGGCATTGTTCTGTGCATATATCAT CTCAATATTCATTATCTGAAAATTATGAATTAGGTACAAAGCTCAAATAATTTATTTTTTCAGGTTAGCAAGAACTT TTTTTTTTTTTTTTTCTGAGATGGAGCATTGCTATGGTTGCCCAGGCTGGAGTGCAATGGCATGATCCAGGCTCACT GCAACATCTGCCTCCCAGGTTCAAGCGATTCTCCTGCCTCAGCCTCCCAAGTAGCTGGCATTACAGGCATGTGCCAC CACCATGCCTGGCTAATTTTCTATTTTTAGTAGATAGGGGGTTTCACCATGTTGGTCAGGCTGATCTCGAACTCCTA ACATCAGGTGATCCACCCTCCTCGGCCTCTGAATGTACTGGGATCACAGGCGTGAGCCACCACACCCAGCCAAGAAT GTGAATTTTGTAGAAGGATATAACCCATATTTCTCTGACCCTAGAGTCCTTAGTATACCTCCCATACCATGTGGCTC ATCCTCCTTACATACATTTCCCATCTTTCACCCTACCTTTTCCTTTTTGTTTCAGCTTTTCACTGTGTGTCAAAATC TAGAACCTTATCTCCTACCTGCTCTGAAACCAACAGCAAGTTGACTTCCATTCTAACCCACATTGGCATTACACTAA TTAAAATCGATACTGAGTTCTAAAATCATCTGGGATTTTGGGGACTATGTCTTACTTCATACTTCCTTGAGATTTCA CATTAAATGTTGGTGTTCATTAAAGGTCCTTCATTTAACTTTGTATTCATCACACTCTTGGATTCACAGTTATATCT AAACTCTTATATATAGCCTGTATAATCCCAATTCCCAAGTCTGATTTCTAACCTCTGACCTCCAACCTCAGTGCCAA ACCCATATATCAAACAATGTACTGGGCTTATTTATATAGATGTCCTATAGGCACCTCAGACTCAGCATGGGTATTTC ACTTGTTATACTAAAACTGTTTCTCTTCCAGTGTTTTCCATTTTAGTCATTAGATAGCTACTTGCCCATTCACCAAG GTCACAGATTAAAATCATTTCCCTACCTCTAATCAACAGTTCAATTCTGCTTCAATTTGTCCCTATCTATTAATCAC CACTCTTACTGCCCAGTCAGGTCCTCATTGTTTCCTGAACAAGAGTAGATGCTATTCTTTCCACTTTAAGACCTTAT CCTGGCTGGATGCGGTGGCTCAGGCTTGTAAACCCAGCACTTTGGGAGGCCGAGGCAGGCAGATCACTTGAGGTCAG GAGTTCAAGACCAGCCTGACCAACATGGTGAAACCCCATCTCTACTAAAAATACAAAATCAGCCGGGCGTGTGGTGC ATGCCTGCAGTCCCAGCTATTCAGGTGGCTGAGGCAGGAGAATTGCTTGAACCCAGGAGGCGGAGGTTGCGGTGAGC CTAGATTGCACCATTGCACTCTAGCTTGGGCAATAGGGATGAAACTCCATCTCAGAAGAGAAAAGAAAAAAAGACCT TATTCTGTTACACAAATCCTCTCAATGCAATCCATATAGAATAAACATGTAACCAGATCTCCCAATGTGTAAAATCA TTTCAGGTAGAACAGAATTAAAGTGAAAAGCCAAGTCTTTGGAATTAACAGACAAAGTTCAAATAACAGTCCTCATG GCCTTAAGAATTTACCTAACATTTTTTTTAGAATCAATTTTCTTATATATGAATTGGAAACATAATTCCTCCCTCAC AAACACATTCTAAGATTTTAAGGAGATATTGATGAAGTACATCATCTGTCATTTTTAACAGTTAGTGGTAGTGATTC ACACAGCACATTATGATCTGTTCTTGTATGTTCTGTTCCATTCTGTATTCTTGACCTGGTTGTATTCTTTCTGAGCT CCAGATCCACATATCTAAGTACATCTTTTTGCATTTTACAAGAGTGCATACAATACAATGTATCCAAGACTGTATTT CTGATTTTATCGTACCACTAAACTCACAAATGTGGCCCTATTCTTGTGTTCACGACTGACATCACCGTCATGGTCCA AGTCTGATAATAGAAATGGCATTGTCACTTTCTTCCCTACTGCAACAGAAGCCCAGCTATTTGTCTCCCATTTTCTC TACTTCTAAAATACATTTCTTCACTAAGTGAGAATAATCTTTTAAAGACACAAATCAAACCATGCCACCACCTTTCT TGAATTATTCAATATCTTTCGTTGGCTTCCAGGTTACAGAAAAATAACTTGTAACAAAGTTTAAAGGTCATTCATGG CTCCTCTCTACCCTATTTTATAACATTTCCCCTTGTGATCAGAATCTCAGGCACATCATCCATCTTTCTATATACAA ATAAAGTCATATAGTTTGAACTCACCTCTGGTTACTTTTAATCAACCAAATGCTGTAAAATGCATTTGTATCGCTAC GTGTTAAGCAGTAGTTGATTCTTTTCATTTCTTGTTAATATTCTATTCTTTGACTATACCGTAATTTATCAATTCTA CTGTTGGTAAGCATTTAAGTGGCTACCGGTTTGAGGTTTTTATGATTATTGCTGTCATAAGCATTTCTATACATGTC TTTGGATACACACATGCATGTGTTTCTGAATATCTAAAAATGTAATTGCTAGGTAATAGACTTATCAAGCATCCAGC ATTTGTGGATACTATTAAAGGTTTTCCAAAGGGGTTATACTATTGTACAGTGTCACCAACAGAGTTTGAGTTTCTAT TGATCCATATCACCACCAAAATTTGAACTGTCAGTCTTATCTCTTCTCTTGTCTCTTTTTTCCTCTTTTTTTTCCTT CCCTTCCCCTCTCTTCGTTTCTTTTCTCTCCTCTTCTCTTCTTTCCTCTCTTCCCTTCCCTTTCTCTTTCTCTTCCC TATCCCTTCTCCTCTCCTCTCCCCTCCTTTTTTCTCCTCTCCTCTCCATTATTTATTTTTCCTTCTTCTCCTCCATC CCTTCCATCCTCTCTCTTCCCCTCTTCCTTCCTTCCTTTCTCCATTTCTTCCTCCTCTTTCCCTCAATCCTTCCTTT TGGATATGCTCATGGGTGTGTATTTGTCTGCCATTGTGGCATTATTTGAATTCAGAAAAGAGTGAAAAACTACTGGG ATCTTCATTCTGGGTCTAATTCCACATTTTTTTTTAAGAACACACTCTGTAAAAATGTTCTGTACTAGCATATTCCC AGGAACTTCGTTAAATTTAATCTGGCTGAATATGGTAAATCTACTTTGCACTTTGCATTCTTTCTTTAGTCATACCA TAATTTTAAACATTCAAAATATTTGTATATAATATTTGATTTTATCTGTCATTAAAATGTTAACCTTAAAATTCATG TTTCCAGAACCTATTTCAATAACTGGTAAATAAACACTATTCATTTTTTAAATATTCTTTTAATGGATATTTATTTC AATATAATAAAAAATTAGAGTTTTATTATAGGAAGAATTTACCAAAAGAAGGAGGAAGCAAGCAAGTTTAAACTGCA GCAATAGTTGTCCATTCCAACCTCTCAAAATTCCCTTGGAGACAAAATCTCTAGAGGCAAAGAAGAACTTTATATTG AGTCAACTTGTTAAAACATCTGCTTTTAGATAAGTTTTCTTAGTATAAAGTGACAGAAACAAATAAGTTAAACTCTA AGATACATTCCACTATATTAGCCTAAAACACTTCTGCAAAAATGAAACTAGGAGGATATTTTTAGAAACAACTGCTG AAAGAGATGCGGTGGGGAGATATGCAGAGGAGAACAGGGTTTCTGAGTCAAGACACACATGACAGAACAGCCAATCT CAGGGCAAGTTAAGGGAATAGTGGAATGAAGGTTCATTTTTCATTCTCACAAACTAATGAAACCCTGCTTATCTTAA ACCAACCTGCTCACTGGAGCAGGGAGGACAGGACCAGCATAAAAGGCAGGGCAGAGTCGACTGTTGCTTACACTTTC TTCTGACATAACAGTGTTCACTAGCAACCTCAAACAGACACCATGGTGCATCTGACTCCTGAGGAGAAGACTGCTGT CAATGCCCTGTGGGGCAAAGTGAACGTGGATGCAGTTGGTGGTGAGGCCCTGGGCAGGTTGGTATCAAGGTTATAAG AGAGGCTCAAGGAGGCAAATGGAAACTGGGCATGTGTAGACAGAGAAGACTCTTGGGTTTCTGATAGGCACTGACTC TCTGTCCCTTGGGCTGTTTTCCTACCCTCAGATTACTGGTGGTCTACCCTTGGACCCAGAGGTTCTTTGAGTCCTTT GGGGATCTGTCCTCTCCTGATGCTGTTATGGGCAACCCTAAGGTGAAGGCTCATGGCAAGAAGGTGCTAGGTGCCTT TAGTGATGGCCTGGCTCACCTGGACAACCTCAAGGGCACTTTTTCTCAGCTGAGTGAGCTGCACTGTGACAAGCTGC ACGTGGATCCTGAGAACTTCAGGGTGAGTCCAGGAGATGCTTCACTTTTCTCTTTTTACTTTCTAATCTTACATTTT GGTTCTTTTACCTACCTGCTCTTCTCCCACATTTTTGTCATTTTACTATATTTTATCATTTAATGCTTCTAAAATTT TGTTAATTTTTTATTTAAATATTCTGCATTTTTTCCTTCCTCACAATCTTGCTATTTTAAATTATTTAATATCCTGT CTTTCTCTCCCAACCCCCTCCCTTCATTTTTCCTTCTCTAACAACAACTCAAATTATGCATACCAGCTCTCACCTGC TAATTCTGCACTTAGAATAATCCTTTTGTCTCTCCACATGGGTATGGGAGAGGCTCCAACTCAAAGATGAGAGGCAT AGAATACTGTTTTAGAGGCTATAAATCATTTTACAATAAGGAATAATTGGAATTTTATAAATTCTGTAGTAAATGGA ATGGAAAGGAAAGTGAATATTTGATTATGAAAGACTAGGCAGTTACACTGGAGGTGGGGCAGAAGTCGTTGCTAGGA GACAGCCCATCATCACACTGATTAATCAATTAATTTGTATCTATTAATCTGTTTATAGTAATTAATTTGTATATGCT ATATACACATACAAAATTAAAACTAATTTGGAATTAATTTGTATATAGTATTATACAGCATATATAGCATATATGTA CATATATAGACTACATGCTAGTTAAGTACATAGAGGATGTGTGTGTATAGATATATGTTATATGTATGCATTCATAT ATGTACTTATTTATGCTGATGGGAATAACCTGGGGATCAGTTTTGTCTAAGATTTGGGCAGAAAAAAATGGGTGTTG GCTCAGTTTCTCAGAAGCCAGTCTTTATTTCTCTGTTAACCATATGCATGTATCTGCCTACCTCTTCTCCGCAGCTC TTGGGCAATGTGCTGGTGTGTGTGCTGGCCCGCAACTTTGGCAAGGAATTCACCCCACAAATGCAGGCTGCCTATCA GAAGGTGGTGGCTGGTGTGGCTAATGCCCTGGCTCACAAGTACCATTGAGATCCTGGACTGTTTCCTGATAACCATA AGAAGACCCTATTTCCCTAGATTCTATTTTCTGAACTTGGGAACACAATGCCTACTTCAAGGGTATGGCTTCTGCCT AATAAAGAATGTTCAGCTCAACTTCCTGATTAATTTCACTTATTTCATTTTTTTGTCCAGGTGTGTAAGAAGGTTCC TGAGGCTCTACAGATAGGGAGCACTTGTTTATTTTACAAAGAGTACATGGGAAAAGAGAAAAGCAAGGGAACCGTAC AAGGCATTAATGGGTGACACTTCTACCTCCAAAGAGCAGAAATTATCAAGAACTCTTGATACAAAGATAATACTGGC ACTGCAGAGGTTCTAGGGAAGACCTCAACCCTAAGACATAGCCTCAAGGGTAATAGCTACGATTAAACTCCAACAAT TACTGAGAAAATAATGTGCTCAATTAAAGGCATAATGATTACTCAAGACAATGTTATGTTGTCTTTCTTCCTCCTTC CTTTGCCTGCACATTGTAGCCCATAATACTATACCCCATCAAGTGTTCCTGCTCCAAGAAATAGCTTCCTCCTCTTA CTTGCCCCAGAACATCTCTGTAAAGAATTTCCTCTTATCTTCCCATATTTCAGTCAAGATTCATTGCTCACGTATTA CTTGTGACCTCTCTTGACCCCAGCCACAATAAACTTCTCTATACTACCCAAAAAATCTTTCCAAACCCTCCCCGACA CCATATTTTTATATTTTTCTTATTTATTTCATGCACACACACACACTCCGTGCTTTATAAGCAATTCTGCCTATTCT CTACCTTCTTACAATGCCTACTGTGCCTCATATTAAATTCATCAATGGGCAGAAAGAAAATATTTATTCAAGAAAAC AGTGAATGAATGAACGAATGAGTAAATGAGTAAATGAAGGAATGATTATTCCTTGCTTTAGAACTTCTGGAATTAGA GGACAATATTAATAATACCATCGCACAGTGTTTCTTTGTTGTTAATGCTACAACATACAAAGAGGAAGCATGCAGTA AACAACCGAACAGTTATTTCCTTTCTGATCATAGGAGTAATATTTTTTTCCTTGAGCACATTTTTGCCATAGGTAAA ATTAGAAGGATTTTTAGAACTTTCTCAGTTGTATACATTTTTAAAAATCTGTATTATATGCATGTTGATTAATTTTA AACTTACTTGAATACCTAAACAGAATCTGTTGTTTCCTTGTGTTTGAAAGTGCTTTCACAGTAACTCTGTCTGTACT GCCAGAATATACTGACAATGTGTTATAGTTAACTGTTTTGATCACAACATTTTGAATTGACTGGCAGCAGAAGCTCT TTTTATATCCATGTGTTTTCCTTAAGTCATTATACATAGTAGGCATGAGACTCTTTATACTGAATAAGATATTTAGG AACCACTGGTTTACATATCAGAAGCAGAGCTACTCAGGGCATTTTGGGGAAGATCACTTTCACATTCCTGAGCATAG GGAAGTTCTCATAAGAGTAAGATATTAAAAGGAGATACTTGTGTGGTATTCGAAAGACAGTAAGAGAGATTGTAGAC CTTATGATCTTGATAGGGAAAACAAACTACATTCCTTTCTCCAAAAGTCAAAAAAAAAGAGCAAATATAGCTTACTA TACCTTCTATTCCTACACCATTAGAAGTAGTCAGTGAGTCTAGGCAAGATGTTGGCCCTAAAAATCCAAATACCAGA GAATTCATGAGAACATCACCTGGATGGGACATGTGCCGAGCAACACAATTACTATATGCTAGGCATTGCTATCTTCA TATTGAAGATGAGGAGGTCAAGAGATGAAAAAAGACTTGGCACCTTGTTGTTATATTAAAATTATTTGTTAGAGTAG AGCTTTTGTAAGAGTCTAGGAGTGTGGGAGCTAAATGATGATACACATGGACACAAAGAATAGATCAACAGACACCC AGGCCTACTTGAGGGTTGAGGGTGGGAAGAGGGAGACGATGAAAAAGAACCTATTGGGTATTAAGTTCATCACTGAG TGATGAAATAATCTGTACATCAAGACCCAGTGATATGCAATTTACCTATATAACTTGTACATGTACCCCCAAATTTA AAATAAAGTTAAAACAAAGTATAGGAATGGAATTAATTCCTCAAGATTTGGCTTTAATTTTATTTGATAATTTATCA AATGGTTGTTTTTCTTTTCTCACTATGGCGTTGCTTTATAAACTATGTTCAGTATGTCTGAATGAAAGGGTGTGTGT GTGTGTGAAAGAGAGGGAGAGAGGAAGGGAAGAGAGGACGTAATAATGTGAATTTGAGTTCATGAAAATTTTTCAAT AAAATAATTTAATGTCAGGAGAATTAAGCCTAATAGTCTCCTAAATCATCCATCTCTTGAGCTTCAGAGCAGTCCTC TGAATTAATGCCTACATGTTTGTAAAGGGTGTTCAGACTGAAGCCAAGATTCTACCTCTAAAGAGATGCAATCTCAA ATTTATCTGAAGACTGTACCTCTGCTCTCCATAAATTGACACCATGGCCCACTTAATGAGGTTAAAAAAAAGCTAAT TCTGAATGAAAATCTGAGCCCAGTGGAGGAAATATTAATGAACAAGGTGCAGACTGAAATATAAATTTTCTGTAATA ATTATGCATATACTTTAGCAAAGTTCTGTCTATGTTGACTTTATTGCTTTTGGTAAGAAATACAACTTTTTAAAGTG AACTAAACTATCCTATTTCCAAACTATTTTGTGTGTGTGCGGTTTGTTTCTATGGGTTCTGGTTTTCTTGGAGCATT TTTATTTCATTTTAATTAATTAATTCTGAGAGCTGCTGAGTTGTGTTTACTGAGAGATTGTGTATCTGCGAGAGAAG TCTGTAGCAAGTAGCTAGACTGTGCTTGACCTAGGAACATATACAGTAGATTGCTAAAATGTCTCACTTGGGGAATT TTAGACTAAACAGTAGAGCATGTATAAAAATACTCTAGTCAAGTGCTGCTTTTGAAACAAATGATAAAACCACACTC CCATAGATGAGTGTCATGATTTTCATGGAGGAAGTTAATATTCATCCTCTAAGTATACCCAGACTAGGGCCATTCTG ATATAAAACATTAGGACTTAAGAAAGATTAATAGACTGGAGTAAAGGAAATGGACCTCTGTCTCTCTCGCTGTCTCT TTTTTGAGGACTTGTGTGTGTGTGTGTGTGTGTGTGTGTGTGTGTTGTGGTCAGTGGGGCTGGAATAAAAGTAGAAT AGACCTGCACCTGCTGTGGCATCCATTCACAGAGTAGAAGCAAGCTCACAATAGTGAAGATGTCAGTAAGCTTGAAT AGTTTTTCAGGAACTTTGAATGCTGATTTAGATTTGAAACTGAGGCTCTGACCATAACCAAATTTGCACTATTTATT GCTTCTTGAAACTTATTTGCCTGGTATGCCTGGGCTTTTGATGGTCTTAGTATAGCTTGCAGCCTTGTCCCTGCAGG GTATTATGGGTAATAGAAAGAAAAGTCTGCGTTACACTCTAGTCACACTAAGTAACTACCATTGGAAAAGCAACCCC TGCCTTGAAGCCAGGATGATGGTATCTGCAGCAGTTGCCAACACAAGAGAAGGATCCATAGTTCATCATTTAAAAAA GAAAACAAAATAGAAAAAGGAAAACTATTTCTGAGCATAAGAAGTTGTAGGGTAAGTCTTTAAGAAGGTGACAATTT CTGCCAATCAGGATTTCAAAGCTCTTGCTTTGACAATTTTGGTCTTTCAGAATACTATAAATATAACCTATATTATA ATTTCATAAAGTCTGTGCATTTTCTTTGACCCAGGATATTTGCAAAAGACATATTCAAACTTCCGCAGAACACTTTA TTTCACATATACATGCCTCTTATATCAGGGATGTGAAACAGGGTCTTGAAAACTGTCTAAATCTAAAACAATGCTAA TGCAGGTTTAAATTTAATAAAATAAAATCCAAAATCTAACAGCCAAGTCAAATCTGTATGTTTTAACATTTAAAATA TTTTAAAGACGTCTTTTCCCAGGATTCAACATGTGAAATCTTTTCTCAGGGATACACGTGTGCCTAGATCCTCATTG CTTTAGTTTTTTACAGAGGAATGAATATAAAAAGAAAATACTTAAATTTTATCCCTCTTACCTCTATAATCATACAT AGGCATAATTTTTTAACCTAGGCTCCAGATAGCCATAGAAGAACCAAACACTTTCTGCGTGTGTGAGAATAATCAGA GTGAGATTTTTTCACAAGTACCTGATGAGGGTTGAGACAGGTAGAAAAAGTGAGAGATCTCTATTTATTTAGCAATA ATAGAGAAAGCATTTAAGAGAATAAAGCAATGGAAATAAGAAATTTGTAAATTTCCTTCTGATAACTAGAAATAGAG GATCCAGTTTCTTTTGGTTAACCTAAATTTTATTTCATTTTATTGTTTTATTTTATTTTATTTTATTTTATTTTGTG TAATCGTAGTTTCAGAGTGTTAGAGCTGAAAGGAAGAAGTAGGAGAAACATGCAAAGTAAAAGTATAACACTTTCCT TACTAAACCGACTGGGTTTCCAGGTAGGGGCAGGATTCAGGATGACTGACAGGGCCCTTAGGGAACACTGAGACCCT ACGCTGACCTCATAAATGCTTGCTACCTTTGCTGTTTTAATTACATCTTTTAATAGCAGGAAGCAGAACTCTGCACT TCAAAAGTTTTTCCTCACCTGAGGAGTTAATTTAGTACAAGGGGAAAAAGTACAGGGGGATGGGAGAAAGGCGATCA CGTTGGGAAGCTATAGAGAAAGAAGAGTAAATTTTAGTAAAGGAGGTTTAAACAAACAAAATATAAAGAGAAATAGG AACTTGAATCAAGGAAATGATTTTAAAACGCAGTATTCTTAGTGGACTAGAGGAAAAAAATAATCTGAGCCAAGTAG AAGACCTTTTCCCCTCCTACCCCTACTTTCTAAGTCACAGAGGCTTTTTGTTCCCCCAGACACTCTTGCAGATTAGT CCAGGCAGAAACAGTTAGATGTCCCCAGTTAACCTCCTATTTGACACCACTGATTACCCCATTGATAGTCACACTTT GGGTTGTAAGTGACTTTTTATTTATTTGTATTTTTGACTGCATTAAGAGGTCTCTAGTTTTTTATCTCTTGTTTCCC AAAACCTAATAAGTAACTAATGCACAGAGCACATTGATTTGTATTTATTCTATTTTTAGACATAATTTATTAGCATG CATGAGCAAATTAAGAAAAACAACAACAAATGAATGCATATATATGTATATGTATGTGTGTATATATACACATATAT ATATATATTTTTTTTCTTTTCTTACCAGAAGGTTTTAATCCAAATAAGGAGAAGATATGCTTAGAACTGAGGTAGAG TTTTCATCCATTCTGTCCTGTAAGTATTTTGCATATTCTGGAGACGCAGGAAGAGATCCATCTACATATCCCAAAGC TGAATTATGGTAGACAAAGCTCTTCCACTTTTAGTGCATCAATTTCTTATTTGTGTAATAAGAAAATTGGGAAAACG ATCTTCAATATGCTTACCAAGCTGTGATTCCAAATATTACGTAAATACACTTGCAAAGGAGGATGTTTTTAGTAGCA ATTTGTACTGATGGTATGGGGCCAAGAGATATATCTTAGAGGGAGGGCTGAGGGTTTGAAGTCCAACTCCTAAGCCA GTGCCAGAAGAGCCAAGGACAGGTACGGCTGTCATCACTTAGACCTCACCCTGTGGAGCCACACCCTAGGGTTGGCC AATCTACTCCCAGGAGCAGGGAGGGCAGGAGCCAGGGCTGGGCATAAAAGTCAGGGCAGAGCCATCTATTGCTTACA TTTGCTTCTGACACAACTGTGTTCACTAGCAACCTCAAACAGACACCATGGTGCACCTGACTCCTGAGGAGAAGTCT GCCGTTACTGCCCTGTGGGGCAAGGTGAACGTGGATGAAGTTGGTGGTGAGGCCCTGGGCAGGTTGGTATCAAGGTT ACAAGACAGGTTTAAGGAGACCAATAGAAACTGGGCATGTGGAGACAGAGAAGACTCTTGGGTTTCTGATAGGCACT GACTCTCTCTGCCTATTGGTCTATTTTCCCACCCTTAGGCTGCTGGTGGTCTACCCTTGGACCCAGAGGTTCTTTGA GTCCTTTGGGGATCTGTCCACTCCTGATGCTGTTATGGGCAACCCTAAGGTGAAGGCTCATGGCAAGAAAGTGCTCG GTGCCTTTAGTGATGGCCTGGCTCACCTGGACAACCTCAAGGGCACCTTTGCCACACTGAGTGAGCTGCACTGTGAC AAGCTGCACGTGGATCCTGAGAACTTCAGGGTGAGTCTATGGGACCCTTGATGTTTTCTTTCCCCTTCTTTTCTATG GTTAAGTTCATGTCATAGGAAGGGGAGAAGTAACAGGGTACAGTTTAGAATGGGAAACAGACGAATGATTGCATCAG TGTGGAAGTCTCAGGATCGTTTTAGTTTCTTTTATTTGCTGTTCATAACAATTGTTTTCTTTTGTTTAATTCTTGCT TTCTTTTTTTTTCTTCTCCGCAATTTTTACTATTATACTTAATGCCTTAACATTGTGTATAACAAAAGGAAATATCT CTGAGATACATTAAGTAACTTAAAAAAAAACTTTACACAGTCTGCCTAGTACATTACTATTTGGAATATATGTGTGC TTATTTGCATATTCATAATCTCCCTACTTTATTTTCTTTTATTTTTAATTGATACATAATCATTATACATATTTATG GGTTAAAGTGTAATGTTTTAATATGTGTACACATATTGACCAAATCAGGGTAATTTTGCATTTGTAATTTTAAAAAA TGCTTTCTTCTTTTAATATACTTTTTTGTTTATCTTATTTCTAATACTTTCCCTAATCTCTTTCTTTCAGGGCAATA ATGATACAATGTATCATGCCTCTTTGCACCATTCTAAAGAATAACAGTGATAATTTCTGGGTTAAGGCAATAGCAAT ATTTCTGCATATAAATATTTCTGCATATAAATTGTAACTGATGTAAGAGGTTTCATATTGCTAATAGCAGCTACAAT CCAGCTACCATTCTGCTTTTATTTTATGGTTGGGATAAGGCTGGATTATTCTGAGTCCAAGCTAGGCCCTTTTGCTA ATCATGTTCATACCTCTTATCTTCCTCCCACAGCTCCTGGGCAACGTGCTGGTCTGTGTGCTGGCCCATCACTTTGG CAAAGAATTCACCCCACCAGTGCAGGCTGCCTATCAGAAAGTGGTGGCTGGTGTGGCTAATGCCCTGGCCCACAAGT ATCACTAAGCTCGCTTTCTTGCTGTCCAATTTCTATTAAAGGTTCCTTTGTTCCCTAAGTCCAACTACTAAACTGGG GGATATTATGAAGGGCCTTGAGCATCTGGATTCTGCCTAATAAAAAACATTTATTTTCATTGCAATGATGTATTTAA ATTATTTCTGAATATTTTACTAAAAAGGGAATGTGGGAGGTCAGTGCATTTAAAACATAAAGAAATGAAGAGCTAGT TCAAACCTTGGGAAAATACACTATATCTTAAACTCCATGAAAGAAGGTGAGGCTGCAAACAGCTAATGCACATTGGC AACAGCCCTGATGCCTATGCCTTATTCATCCCTCAGAAAAGGATTCAAGTAGAGGCTTGATTTGGAGGTTAAAGTTT TGCTATGCTGTATTTTACATTACTTATTGTTTTAGCTGTCCTCATGAATGTCTTTTCACTACCCATTTGCTTATCCT GCATCTCTCAGCCTTGACTCCACTCAGTTCTCTTGCTTAGAGATACCACCTTTCCCCTGAAGTGTTCCTTCCATGTT TTACGGCGAGATGGTTTCTCCTCGCCTGGCCACTCAGCCTTAGTTGTCTCTGTTGTCTTATAGAGGTCTACTTGAAG AAGGAAAAACAGGGGGCATGGTTTGACTGTCCTGTGAGCCCTTCTTCCCTGCCTCCCCCACTCACAGTGACCCGGAA TCTGCAGTGCTAGTCTCCCGGAACTATCACTCTTTCACAGTCTGCTTTGGAAGGACTGGGCTTAGTATGAAAAGTTA GGACTGAGAAGAATTTGAAAGGGGGCTTTTTGTAGCTTGATATTCACTACTGTCTTATTACCCTATCATAGGCCCAC CCCAAATGGAAGTCCCATTCTTCCTCAGGATGTTTAAGATTAGCATTCAGGAAGAGATCAGAGGTCTGCTGGCTCCC TTATCATGTCCCTTATGGTGCTTCTGGCTCTGCAGTTATTAGCATAGTGTTACCATCAACCACCTTAACTTCATTTT TCTTATTCAATACCTAGGTAGGTAGATGCTAGATTCTGGAAATAAAATATGAGTCTCAAGTGGTCCTTGTCCTCTCT CCCAGTCAAATTCTGAATCTAGTTGGCAAGATTCTGAAATCAAGGCATATAATCAGTAATAAGTGATGATAGAAGGG TATATAGAAGAATTTTATTATATGAGAGGGTGAAACCTAAAATGAAATGAAATCAGACCCTTGTCTTACACCATAAA CAAAAATAAATTTGAATGGGTTAAAGAATTAAACTAAGACCTAAAACCATAAAAATTTTTAAAGAAATCAAAAGAAG AAAATTCTAATATTCATGTTGCAGCCGTTTTTTGAATTTGATATGAGAAGCAAAGGCAACAAAAGGAAAAATAAAGA AGTGAGGCTACATCAAACTAAAAAATTTCCACACAAAAAAGAAAACAATGAACAAATGAAAGGTGAACCATGAAATG GCATATTTGCAAACCAAATATTTCTTAAATATTTTGGTTAATATCCAAAATATATAAGAAACACAGATGATTCAATA ACAAACAAAAAATTAAAAATAGGAAAATAAAAAAATTAAAAAGAAGAAAATCCTGCCATTTATGCGAGAATTGATGA ACCTGGAGGATGTAAAACTAAGAAAAATAAGCCTGACACAAAAAGACAAATACTACACAACCTTGCTCATATGTGAA ACATAAAAAAGTCACTCTCATGGAAACAGACAGTAGAGGTATGGTTTCCAGGGGTTGGGGGTGGGAGAATCAGGAAA CTATTACTCAAAGGGTATAAAATTTCAGTTATGTGGGATGAATAAATTCTAGATATCTAATGTACAGCATCGTGACT GTAGTTAATTGTACTGTAAGTATATTTAAAATTTGCAAAGAGAGTAGATTTTTTTGTTTTTTTAGATGGAGTTTTGC TCTTGTTGTCCAGGCTGGAGTGCAATGGCAAGATCTTGGCTCACTGCAACCTCCGCCTCCTGGGTTCAAGCAAATCT CCTGCCTCAGCCTCCCGAGTAGCTGGGATTACAGGCATGCGACACCATGCCCAGCTAATTTTGTATTTTTAGTAGAG ACGGGGTTTCTCCATGTTGGTCAGGCTGATCCGCCTCCTCGGCCACCAAAGGGCTGGGATTACAGGCGTGACCACCG GGCCTGGCCGAGAGTAGATCTTAAAAGCATTTACCACAAGAAAAAGGTAACTATGTGAGATAATGGGTATGTTAATT AGCTTGATTGTGGTAATCATTTCACAAGGTATACATATATTAAAACATCATGTTGTACACCTTAAATATATACAATT TTTATTTGTGAATGATACCTCAATAAAGTTGAAGAATAATAAAAAAGAATAGACATCACATGAATTAAAAAACTAAA AAATAAAAAAATGCATCTTGATGATTAGAATTGCATTCTTGATTTTTCAGATACAAATATCCATTTGACTGTTTACT CTTTTCCAAAACAATACAATAAATTTTAGCACTTTATCTTCATTTTCCCCTTCCCAATCTATAATTTTATATATATA TATTTTAGATATTTTGTATAGTTTTACTCCCTAGATTTTCTAGTGTTATTATTAAATAGTGAAGAAATGTTTACACT TATGTACAAAATGTTTTGCATGCTTTTCTTCATTTCTAACATTCTCTCTAAGTTTATTCTATTTTTTCCTGATTATC CTTAATATTATCTCTTTCTGCTGGAAATATATTGTTACTTTTGGTTTATCTAAAAATGGCTTCATTTTCTTCATTCT AAAATCATGTTAAATTAATACCACTCATGTGTAAGTAAGATAGTGGAATAAATAGAAATCCAAAAACTAAATCTCAC AAAATATAATAATGTGATATATAAAAATATAGCTTTTAAATTTAGCTTGGAAATAAAAAACAAACAGTAATTGAACA ACTATACTTTTTGAAAAGAGTAAAGTGAAATGCTTAACTGCATATACCACAATCGATTACACAATTAGGTGTGAAGG TAAAATTCAGTCACGAAAAAACTAGAATAAAAATATGGGAAGACATGTATATAATCTTAGAGATAACAGTGTTATTT AATTATCAACCCAAAGTAGAAACTATCAAGGGAGAAATAAATTCAGTCAACAATAAAAGCATTTAAGAAGTTATTCT AGGCTGGGAGCGGTGGCTCACACCTGCAATTGCAGCACTTTGGGAGGCCTAGACAGGCGGATCACGACGTCAGGAGT TCAAGATCAGCCTGGCCAACATAGTGAAACCTCATCGCTACTAAAAATATAAAAACTTAGCCTGGCGTGGTGGCAGG CATGTGTAATCCCAGCAATTTGGGAGGCTGAGGCAGGAGAATCGCTTGATCCTGGGAGGCAGAGGTTGCAGTGAGCC AAGATTGTGCCACTGCATTCCAGCCCAGGTGACAGCATGAGACTCCGTCACAAAAAAAAAAGAAAAAAAAGGGGGGG GGGGGCGGTGGAGCCAAGATGACCGAATAGGAACAGCTCCAGTCTATAGCTCCCATCGTGAGTGACGCAGAAGACGG GTGATTTCTGCATTTCCAACTGAGGTACCAGGTTCATCTCACAGGGAAGTGCCAGGCAGTGGGTGCAGGACAGTAGT GCAGTGCACTGTGCATGAGCCGAAGCAGGGCGAGGCATCACCTCACCCGGGAAGCACAAGGGGTCAGGGAATTCCCT TTCCTAGTCAAAGAAAAGGGTGACAGATGGCACCTGGAAAATCGGGTCACTCCCGCCCTAATACTGCGCTCTTCCAA CAAGCTTAACAAATGGCACACCAGGAGATTATATCCCATGCCTGGCTCAGAGGGTCCTACGCCCATGGAGCCTCGCT CATTGCTAGCACAGCAGTCTGAGGTCAAACTGCAAGGTGGCAGTGAGGCTGGGGGAGGGGTGCCCACCATTGTCCAG GCTTGAGCAGGTAAACAAAGCCGCCTGGAAGCTCGAACTGGGTGGAGCCCACCACAGCTCAAGGAGGCCTGCCTGCC TCTGTAGGCTCCACCTCTAGGGGCAGGGCACAGACAAACAAAAGACAACAAGAACCTCTGCAGACTTAAATGTCCCT GTCTGACAGCTTTGAAGAGAGTAGTGGTTCTCCCAGCACATAGCTTCAGATCTGAGAACAGGCAGACTGCCTCCTCA AGTGGGTCCCTGACCCCCGAGTAGCCTAACTGGGAGGCATCCCCCAGTAGGGCGGACTGACACCTCACATGGCTGGT ACTCCTCTAAGACAAAACTTCCAGAGGAATGATCAGGCAGCAGCATTTGCGGTTCACCAATATCCACTGTTCTGCAG CCACCGCTGCTGATACCCAGGAAAACAGCATCTGGAGTGGACCTCCAGTAAACTCCAACAGACCTGCAGCTGAGGGT CCTGACTGTTAGAAGGAAAACTAACAAACAGAAAGGACATCCACACCAAAAACCCATCTGTACATCACCATCATCAA AGACCAAAGGTAGATAAAACCATAAAGATGGGGAAAAAGCAGAGCAGAAAAACTGGACACTCTAAAAATGAGAGTGC CTCTCCTTCTCCAAAGTAACGCAGCTCCTCACCAGCAATGGAACAAAGCTGGGCAGAGAATGACTTTGACGAGTTGA GAGAGGAAGGCTTCAGAAGATCAAACTACTCCAAGCTAAAGGAGGAAGTTCGAACAAACGGCAAAGAAGTAAAAAAC TTTGAAAAAAAATTAGATGAATGGATAACTAGAATAACCAATGCACAGAAGTCCTTAAAGGACCTGATGGAGCTGAA AACCAAGGCAGGAGAACTACGTGACAAATACACAAGCCTCAGTAACCGATGAGATCAACTGGAAGAAAGGGTATCAA TGACGGAAGATGAAATGAATGAAATGAAGCATGAAGAGAAGTTTAGAGAAAAAAGAATAAAAAGAAACGAACAAAGC CTCCAAGAAATATGGGACTATGTGAAAAGACCAAATCTACATCTAATTGGTGTAGCTGAAAGTGATGGGGAGAATGG AACCAAGTTGGAAAACACTCTGCAGGATATTATCCAGGAGAACTTCCCCAATCTAGCAAGGCAGCCCAAATTCACAT TCAGGAAATACAGAGAACGCCACAAAGATACTCCTAGAGAAAAGCAACTCCAAGACACATAACTGACAGATTCACCA AAGTTGAAATGAAGGAAAAAATGTTAAGGGCAGCCAGAGAGAAAGGTCGGGTTACCCACAAAGGGAAGCCCATCAGA CTAACAGCTGATCTATCGGCAGAAACTCTACAAGCCAGAAGAAAGTGGGGGCCAATATTCAACATTGTTAAAGAAAA GAATTTTCGGCCCAGAATTTCATATCCAGCCAAACTAAGCTTCATAAGCATTGGAGAAATAAAATCCTTTACAGACA AGCAAATGCTGAGAGATTTTGTCACCACCAGGCCTGCCCTACAAGAGCTCCTGAAGGAAGCACTAAACATGGAAAGG AACAACTAGTATCAGCCACTGCAAAAACATGCCAAATTGTAAACGACCATCAAGGCTAGGAAGAAACTGCATCAAGG AGCAAAATAACCAGCTAACATCATAATGACAGGATCAAATTCATACATAACAATACTCACCTTAAATGTAAATAGGC TAAATGCTCCAATTAAAAGACACAGACTGGCAAATTGGATAAGGAGTCAAGACCCATCTGTCGTTATGTATTCAGGA AACCCATCTCACGTGCAGAGACACACATAGGCTCGAAATAAAAGGATGGAGGAATATCTACCAAGCAAATGGAAAAC AAAAAAAGGCAGGGGTTGCAATCCTAGTCTCTGATAAAACAGATTTTAAACCAACAAAGATCAAAAGAGACAAAGAA GGCCATTACATAATGGCAAAGGGATCTATTCAAGAAGAAGAACTAACTATACTAAATATATATGCACCCAATACAGG AGCACCCAGATTCATAAAACAAGTCCTGAGTGACCTACAAAGAGACTTAGATGCCCACACAATAATAATGGGAGACT TTAACACCCCACTGTCAACATTAGACAGATCAACGAGACAGAAAGTTAACAAGGATATCCAGGAATTGGACTCAGCT CTGCACCAAGCAGACCTAATAGACATCTACAGAACTCTCCACCCCAAATCAACAGAATATACATTCTTTTCAGCACC ACACCACACCTATTCCAAAACTGACCACATAGTTGGAAGTAAAGCTCTCCTCAGCAAATGTAAAAGAACAGAAACTA TAACAAACTGTCTCTCAGACCACAGTGCAATCAAACTAGAACTCAGGATTAAGAAACTCACTCAAAACCACTCAGCT ACATGGAAACTGAACAGCCTGCTCCTGAATGACTACTGGGTACATAACAAAATGAAGGCAGAAATAAAGATGTTCTT TGAAACAACGAGAACAAAGACACAACACACCAGAATCTCTGAGACACATTCAAAGCAGTGTGTAGAGGGAAATTTAT AGCACTAAATGCCCACAAGGGAAAGCAGGAAAGATCTAAAATTGACACCCTAACATCACAATTAAAAAACTAGAGAA GCAGGAGCAAACACATTCAAAAGCTAACAGAAGACAAGAAATAACTAAGATCAGAGCAGAAGTGAAGAAGATAGAGA CACAAAAAACCCTTCAAAAAAATCAATGAATCCAGAAGCTGTTTTTTTGAAAAGATCAACAAAATTGATAGACTGCT AGCAAGACTAATAAAGAAGAAAGGGGAGAAGAATCAAATAGACGCAATAAAAAATGACACGGGGTATCACCACTGAT CCCACAGAAATACAAACTACCGTCAGAGAATACTATAAACACCTCTACGCAAATAAACTAGAAAATCTAGAAGAAAT GGATAAATTCCTCGACACATACACTCTGCCAAGACTAAACCAGGAAGAAGTTGTATCTCTGAATAGACCAATAACAG GCTCTGAAATTGAGGCAATAATTAATAGCTTATCAACCAAAAAAAGTCCGGGACCAGTAGGATTCATAGCCGAATTC TACCAGAGGTACAAGGAGGAGCTGGTACCATTCCTTCTGAAACTATTCCAATCAATAGAAAAAGAGGGAATCCTCCC TAACTCATTTTATGAGGCCAGCATCATCCTGATACCAAAGCCTGACAGAGACACAACAAAAAAAGAGAATGTTACAC CAATATCCTTGATGAACATCGATGCAAAAATCCTCAATAAAATACTGGCAAACTGAATCCAGCAGCACATCAAAAAG CTTATCCTCCATGATCAAGTGGGCTTCATCCCTGCCATGCAAGGCTGGTTCAACATACGAAATCAATAAACATAATC CAGCATATAAACAGAACCAAAGACACAAACCATATGATTATCTCAATAGATGCAGAAAAGGCCTTTGACAAAATTCA ACAATGCTTCATGCTAAAAACTCTCAATAAATTAGGTATTGATGGGACATATCTCAAAATAATAAGAGCTATCTATG ACAAACCCACAGCCAATATCATACTGAGTGGACAAAAACTGGAAGCATTCCCTTTGAAAACTGGCACAAGGCAGGGA TGCCCTCTCTCACCACTCCTATTCAACATAGTGTTGGAAGTTCTGGCCAGGGCAATCAGGCAGGAGAAGGAAATAAA GGGCATTCAATTAGGAAAAGAGGAAGGTGAAATTGTCCCTGTTTGCAGATGACATGATTGTATATCTAGAAAACCCC ATTGTCTCAGCCCAAAATCTCCTTAAGCTGATAAGCAACTTCAGCAAAGTCTCAGGATATAAAATCAGTGTGCAAAA ATCACAAGTATTCCTATGCACCAATAACAGACAAACAGAGAGCCAAATCATGAGTGAACTCCCATTCACAATTGCTT CAAAGAGAATAAAATACCTAGGAATCCAACTTACAAGGGATGTGAAGGACCTCTTCAAGGAGAACTACAAACCACTG CTCAATGAAATAAAAGAGGATACAAACAAATGGAAGAACATTCCATGCTTATGGGTAGGAAGAATCATATCGTGAAA ATGGTCATACTGCCCAAGGTAATTTATAGATTCAATGCCATCCCCATCAAGCTACCAATGACTTTCTTCACAGAACT GGAAAAAACTACTTTAAAGTTCATATGGAATCAAAAAAGAGCCCACATCACCAAGGCAATCCTAAGCCAAAAGAACA AAGCTGGAGGCATCACGCTACCTGACTTCAAACTATACTACAATGCTACGGTAACCAAAACAGCATGGTACTGGTAC CAAAACAGAGATCTAGACCAATGGAACAGAACAGAGCCCTCAGAAATAATGCCGCATATCTACAACTATCCGATCTT TGACAAACCTGAGAGAAACAAGCAATGGGGAAAGGATTCCCTATTTAATAAATGGTGCTGGGAAAACTGGCTAGCCA TATGTAGAAAGCTGAAACTGGATCCTTCCTTACACCTTATACAAAAATTAATTCAAGATGGATTAAAGACTTAAACA TTAGACCTAAAACCATAAAAACCCTAGAAAAAAACCTAGGCAATACCATTCAGGACATAGGCATGGGCAAGGACTTC ATGTCTAAAACACCAAAACGAATGGCAACAAAAGACAAAATGGACAAACGGGATCTAATTAAACTAAAGAGCTTCTG CACAGCTAAAGAAACTACCATCAGAGTGAACAGGCAACCTACAAAATGGGAGAAAATTTTTGCAATCTACTCATCTG ACAAAGGGCTAATATCCAGAATCTACAATGAACTCAAACAAATTTACAAGAAAAAACAAACAACCCCATCAAAAAGT GGGCAAAGGATATGAACAGACACTTCTCAAAAGAAGACATTTATGTAATCAAAAAACACATGAAAAAATGCTCATCA TCACTAGCCATCAGAGAAATGCAAATCAAAACCACAATGAGATACCATCTCACACCAGTTAGAATGGCGATCATTAA AAAGTCAGGAAACAACAGGTGCTGGAGAGGATGTGGAGAAACAGGAACAACTTTTACACTGTTGGTGGGACTGTAAA CTAGTTCAACCATTGCGGAAGTCAGTGTGGCAATTCCTCAGGAATCTAGAACTAGAAATACCATTTGACCCAGCCAT CCCATTACTGGGTAGATACCCAAAGGATTATAAATCATGCTGCTATAAAGACACATGCACACGTATGTTTATTGCAG CACTATTCACAATAGCAAAGACTTGGAACCAACCCAAATGTCCAACAACGATAGATTGGATTAAGAAAATGTGGCAC ATATACACCATGGAATACTATGCAGCCATAAAAAATGATGAGTTCATGTCCTTTGTAGGGACATGGATGAAGCTGGA AACTATCATTCTCAGCAAACTATCACAAGGACAATAAACCAAACACCGCATGTTCTCACTCATAGGTGGGAATTGAA CAATGAGAACACATGGACACATGAAGAGGAACATCACACTCTGGGGACTGTTATGGGGTGGGGGGCAGGGGCAGGGA TAGCACTAGGAGATATACCTAATGCTAAATGACGAGTTAATGGGTGCAGCACACCAACATGGCACATGTATACATAT ATAACAAACCTGCCGTTGTGCACATGTACCCTAAAACTTGAAGTATAATAATAAAAAAAAGTTATCCTATTAAAACT GATCTCACACATCCGTAGAGCCATTATCAAGTCTTTCTCTTTGAAACAGACAGAAATTTAGTGTTTTCTCAGTCAGT TAAC

NCBI Accession No. P68871 MVHLTPEEKSAVTALWGKVNVDEVGGEALGRLLVVYPWTORFFESFGDLSTPDAVMGNPKVKAHGKKVLGAFSDGLA HLDNLKGTFATLSELHCDKLHVDPENFRLLGNVLVCVLAHHFGKEFT PPVQAAYQKVVAGVANALAHKYH

NCBI Accession No. NP_000509 MVHLTPEEKSAVTALWGKVNVDEVGGEALGRLLVVYPWTORFFESFGDLSTPDAVMGNPKVKAHGKKVLGAFSDGLA HLDNLKGTFATLSELHCDKLHVDPENFRLLGNVLVCVLAHHFGKEFTPPVQAAYOKVVAGVANALAHKYH

GenBank Accession No. ABD57356.1 MNEQAKQLVAVTROPALNAGVGLVLAQLAEVEARQIPGSLAEARAHCLAQGAPDILLVEVENPQTLAADLAALAECC PPQMRLVLLGERGDVTLFRWLISVGVDDYYPAPLDPDALRTGLLRLLGVPLVTSLRKGRVICVVGAAGGVGTSTVAA NLAMALADQHHRQVALLDLNLHHSRHPILLGSDYAPPGEQWWQATDRLDGTLLAHTAHOLGPRLFLFYDEGQELVLG AEQLVAAVNVMAEHYSTLIIDVPDLRTHGLRALLQEADVVLWLHDFSLGALRLLGQCPQGGQAQRRLLVGNHCRGKE GRVPAQELERVCGQPHAAVLPYDHGVFVRAERAGQPLIQQKSKLARALTLLAGELVGAQVTGRGRR

GenBank Accession No. ANW61888.1 MAPKKKRKVMSQFDILCKTPPKVLVRQFVERFERPSGEKIASCAAELTYLCWMITHNGTAIKRATEMSYNTIISNSL SFDIVNKSLOFKYKTOKATILEASLKKLIPAWEFTII PYNGQKHOSDITDIVSSLQLQFESSEEADKGNSHSKKMLK ALLSEGESIWEITEKILNS FEYTSRFTKTKTLYQFLFLATFINCGRESDI KNVDPKS FKLVONKYLGVI IQCLVTET KTSVSRHIYFFSARGRIDPLVYLDEFLRNSEPVLKRVNRTGNSSSNKQEYQLLKDNLVRSYNKALKKNAPYPIFAIK NGPKSHIGRHLMTSFLSMKGLTELTNVVGNWSDKRASAVARTTYTHQITAIPDHYFALVSRYYAYDPISKEMIALKD ETNPIEEWQHIEQLKGSAEGSIRYPAWNGIISQEVLDYLSSYINRRI

GenBank Accession No. AP_000601 MTKRVRLSDSFNPVYPYEDESTSQHPFINPGFISPNGFTQSPDGVLTLKCLTPLTTTGGSLQLKVGGGLTVDDTDGT LQENIRATAPITKNNHSVELSIGNGLETQNNKLCAKLGNGLKFNNGDICIKDSINTLWTGINPPPNCQIVENTNTND GKLTLVLVKNGGLVNGYVSLVGVSDTVNQMFTQKTANIQLRLYFDSSGNLLTEESDLKI PLKNKSSTATSETVASSK AFMPSTTAYPFNTTTRDSENYIHGICYYMTSYDRSLFPLNISIMLNSRMISSNVAYAIQFEWNLNASESPESNIATL TTSPFFFSYITEDDN

GenBank Accession No. NC_011203 (SEQ ID NO: 208) CTATCTATATAATATACCTTATAGATGGAATGGTGCCAACATGTAAATGAGGTAATTTAAAAAAGTGCGCGCTGTGT GGTGATTGGCTGCGGGGTTAACGGCTAAAAGGGGGGCGCGACCGTGGGAAAATGACGTGACTTATGTGGGAGGAGT TATGTTGCAAGTTATTACGGTAAATGTGACGTAAAACGAGGTGTGGTTTGAACACGGAAGTAGACAGTTTTCCCACG CTTACTGACAGGATATGAGGTAGTTTTGGGCGGATGCAAGTGAAAATTCTCCATTTTCGCGCGAAAACTAAATGAGG AAGTGAATTTCTGAGTCATTTCGCGGTTATGCCAGGGTGGAGTATTTGCCGAGGGCCGAGTAGACTTTGACCGTTTA CGTGGAGGTTTCGATTACCGTGTTTTTCACCTAAATTTCCGCGTACGGTGTCAAAGTTCTGTGTTTTTACGTAGGTG TCAGCTGATCGCTAGGGTATTTAAACCTGACGAGTTCCGTCAAGAGGCCACTCTTGAGTGCCAGCGAGAAGAGTTTT CTCCTCCGCGCCGCAAGTCAGTTCTGCGCTTTGAAAATGAGACACCTGCGCTTCCTGCCACAGGAGGTTATCTCCAG TGAGACCGGGATCGAAATACTGGAGTTTGTGGTAAATACCCTAATGGGAGACGACCCGGAACCGCCAGTGCAGCCTT TCGATCCACCTACGCTGCACGATCTGTATGATTTAGAGATAGACGGGCCGGAGGATCCCAATGAGGAAGCTGTGAAT GGGTTTTTTACTGATTCTATGCTGCTAGCTGCTGATGAAGGATTGGACATAAACCCTCCTCCTGAGACACTTGTTAC CCCAGGGGTGGTTGTGGAAAGCGGCATAGGTGGGAAAAAATTGCCTGATCTGGGAGCAGCTGAAATGGACTTGCGTT GTTATGAAGAGGGTTTTCCTCCCAGTGATGATGAAGATGGGGAAACTGAGCAGTCCATCCATACCGCAGTAAATGAG GGAGTAAAAGCTGCCAGCGATGTTTTTAAGTTGGACTGTCCGGAGCTGCCTGGACATGGCTGTAAGTCTTGTGAATT TCACAGGAATAACACTGGAATGAAAGAACTATTGTGCTCGCTTTGCTATATGAGAATGCACTGCCACTTTATTTACA GTAAGTGTATTTAAGTGAAATTTAAAGGAATAGTGTAGCTATTTAATAACTGTTGAATGGTAGATTTATGTTTTTTT CTTGCGATTTTTTGTAGGTCCTGTGTCTGATGATGAGTCACCTTCTCCTGATTCAACTACCTCACCTCCTGAAATTC AGGCGCCCGCACCTGCAAACGTATGCAAGCCCATTCCTGTGAAGCCTAAGCCTGGGAAACGCCCTGCTGTGGATAAG CTTGAGGACTTGTTGGAGGGTGGGGATGGACCTTTGGACCTTAGTACCCGGAAACTGCCAAGGCAATGAGTGCCCTG CAGCTGTGTTTATTTAATGTGACGTCATGTAATAAAATTATGTCAGCTGCTGAGTGTTTTATTACTTCTTGGGTGGG GACTTGGATATATAAGTAGGAGCAGATCTGTGTGGTTAGCTCACAGCAACCTGCTGCCATCCATGGAGGTTTGGGCT ATCTTGGAAGACCTCAGACAGACTAAGCTACTGCTAGAAAACGCCTCGGACGGAGTCTCTGGCCTTTGGAGATTCTG GTTCGGTGGTGATCTAGCTAGGCTAGTGTTTAGGATAAAACAGGACTACAGGGAAGAATTTGAAAAGTTATTGGACG ATAGTCCGGGACTTTTTGAAGCTCTTAACTTGGGTCATCAGGCTCATTTTAAGGAGAAGGTTTTATCAGTTTTAGAT TTTTCTACTCCTGGTAGAACTGCTGCTGCTGTAGCTTTTCTTACTTTTATATTGGATAAATGGATCCGCCAAACTCA CTTCAGCAAGGGATACGTTTTGGATTTCATAGCAGCAGCTTTGTGGAGAACATGGAAGGCTCGCAGGATGAGGACAA TCTTAGATTACTGGCCAGTGCAGCCTCTGGGAGTAGCAGGGATACTGAGACACCCACCGACCATGCCAGCGGTTCTG CAGGAGGAGCAGCAGGAGGACAATCCGAGAGCCGGCCTGGACCCTCCGGTGGAGGAGTAGCTGACCTGTTTCCTGAA CTGCGACGGGTGCTTACTAGGTCTACGACCAGTGGACAGAACAGGGGAATTAAGAGGGAGAGGAATCCTAGTGGGAA TAATTCAAGAACCGAGTTGGCTTTAAGTTTAATGAGCCGCAGGCGTCCTGAAACTGTTTGGTGGCATGAGGTTCAGA GCGAAGGCAGGGATGAAGTTTCAATATTGCAGGAGAAATATTCACTAGAACAACTTAAGACCTGTTGGTTGGAACCT GAGGATGATTGGGAGGTGGCCATTAGGAATTATGCTAAGATATCTCTGAGGCCTGATAAACAATATAGAATTACTAA GAAGATTAATATTAGAAATGCATGCTACATATCAGGGAATGGGGCAGAGGTTATAATAGATACACAAGATAAAGCAG TTTTTAGATGTTGTATGATGGGTATGTGGCCAGGGGTTGTCGGCATGGAAGCAGTAACACTTATGAATATTAGGTTT AAAGGGGATGGGTATAATGGCATTGTATTTATGGCTAACACTAAGCTGATTCTACATGGTTGTAGCTTTTTTGGGTT TAATAATACGTGTGTAGAAGCTTGGGGGCAAGTTAGTGTGAGGGGTTGTAGTTTTTATGCATGCTGGATTGCAACAT CAGGTAGGGTCAAGAGTCAGTTGTCTGTGAAGAAATGCATGTTTGAGAGATGTAATCTTGGCATACTGAATGAAGGT GAAGCAAGGGTCCGCCACTGCGCAGCTACAGAAACTGGCTGCTTCATTCTAATAAAGGGAAATGCCAGTGTGAAGCA TAATATGATCTGTGGACATTCGGATGAGAGGCCTTATCAGATGCTGACCTGCGCTGGTGGACATTGCAATATTCTTG CTACCGTGCATATCGTTTCACATGCACGCAAAAAATGGCCTGTATTTGAACATAATGTGATTACCAAGTGCACCATG CACATAGGTGGTCGCAGGGGAATGTTTATGCCTTACCAGTGTAACATGAATCATGTGAAGGTAATGTTGGAACCAGA TGCCTTTTCCAGAGTGAGCTTAACAGGAATCTTTGATATGAATATTCAACTATGGAAGATCCTGAGATATGATGACA CTAAACCAAGGGTGCGCGCATGCGAATGCGGAGGCAAGCATGCTAGATTCCAGCCGGTGTGCGTGGATGTGACTGAA GACTTGAGACCCGATCATTTGGTGCTTGCCTGCACTGGAGCGGAGTTCGGTTCTAGTGGTGAAGAAACTGACTAAAG TGAGTAGTGGGGCAAGATGTGGATGGGGACTTTCAGGTTGGTAAGGTGGGCAGATTGGGTAAATTTTGTTAATTTCT GTCTTGCAGCTGCCATGAGTGGAAGCGCTTCTTTTGAGGGGGGAGTATTTAGCCCTTATCTGACGGGCAGGCTCCCA CCATGGGCAGGAGTTCGTCAGAATGTCATGGGATCCACTGTGGATGGGAGACCCGTCCAGCCCGCCAATTCCTCAAC GCTGACCTATGCCACTTTGAGTTCGTCACCATTGGATGCAGCTGCAGCCGCCGCCGCTACTGCTGCCGCCAACACCA TCCTTGGAATGGGCTATTATGGAAGCATCGTTGCCAATTCCAGTTCCTCTAATAACCCTTCAACCCTGGCTGAGGAC AAGCTACTTGTTCTCTTGGCGCAGCTCGAGGCCTTAACCCAACGCTTAGGCGAACTGTCTAAGCAGGTGGCCCAGTT GCGTGAGCAAACTGAGTCTGCTGTTGCCACAGCAAAGTCTAAATAAAGATCTCAAATCAATAAATAAAGAAATACTT GATATAAAACAAATGAATGTTTATTTGATTTTTCGCGCGCGGTATGCCCTGGACCATCGGTCTCGATCATTGAGAAC GCGGTGGATCTTTTCCAGTACCCTGTAAAGGTGGGATTGAATGTTTAGATACATGGGCATTAGTCCGTCTCGGGGGT GGAGATAGCTCCATTGAAGAGCCTCTTGCTCCGGGGTAGTGTTATAAATCACCCAGTCATAGCAAGGTCGGAGTGCA TGGTGTTGCACAATATCTTTTAGGAGCAGACTAATTGCAACGGGGAGGCCCTTAGTGTAGGTGTTTACAAATCTGTT GAGCTGGGACGGGTGCATCCGGGGTGAAATTATATGCATTTTGGACTGGATCTTGAGGTTGGCAATGTTGCCGCCTA GATCCCGTCTCGGGTTCATATTGTGCAGGACCACCAAGACAGTGTATCCGGTGCACTTGGGAAATTTATCATGCAGC TTAGAGGGAAAAGCATGAAAAAATTTGGAGACGCCTTTGTGACCCCCCAGATTCTCCATGCACTCATCCATAATGAT AGCGATGGGGCCGTGGGCAGCGGCACGGGCGAACACGTTCCGGGGGTCTGAAACATCATAGTTATGCTCCTGAGTCA GGTCATCATAAGCCATTTTAATAAACTTTGGGCGGAGGGTGCCAGATTGGGGGATGAAAGTTCCCTCTGGCCCGGGA GCATAGTTTCCCTCACATATTTGCATTTCCCAGGCTTTCAGTTCAGAGGGGGGGATCATGTCCACCTGCGGGGCTAT AAAAAATACCGTTTCTGGAGCCGGGGTGATTAACTGGGACGAGAGCAAATTCCTAAGCAGCTGAGACTTGCCGCACC CAGTGGGACCGTAAATGACCCCAATTACGGGTTGCAGATGGTAGTTTAGGGAACGACAGCTGCCGTCCTCCCGGAGC AGGGGGGCCACTTCGTTCATCATTTCCCTTACATGGATATTTTCCCGCACCAAGTCCGTTAGGAGGCGCTCTCCCCC AAGGGATAGAAGCTCCTGGAGCGAGGAGAAGTTTTTCAGCGGCTTCAGCCCGTCAGCCATGGGCATTTTGGAAAGAG TCTGTTGCAAGAGCTCGAGCCGGTCCCAGAGCTCGGTGATGTGCTCTATGGCATCTCGATCCAGCAGACCTCCTCGT TTCGCGGGTTGGGACGGCTCCTGGAGTAGGGAATCAGACGATGGGCGTCCAGCGCTGCCAGGGTCCGATCCTTCCAT GGTCGCAGCGTCCGAGTCAGGGTTGTTTCCGTCACGGTGAAGGGGTGCGCGCCTGGTTGGGCGCTTGCGAGGGTGCG CTTCAGACTCATCCTGCTGGTCGAGAACCGCTGCCGATTGGCGCCCTGCATGTCGGCCAGGTAGCAGTTTACCATGA GTTCGTAGTTGAGCGCCTCGGCCGCGTGGCCTTTGGCACGGAGCTTACCTTTGGAAGTTTTATGGCAGGCGGGGCAG TAGATACATTTGAGGGCATACAACTTGGGCGCGAGGAAAATGGATTCGGGGGAGTATGCATCCGCACCGCAGGAGGC GCAGACGGTTTCGCACTCCACGAGCCAGGTCAGATCCGGCTCATCGGGGTCAAAAACAAGTTTTCCGCCATGTTTTT TGATGCGTTTCTTACCTTTGGTTTCCATGAGTTCGTGTCCCCGCTGGGTGACAAAGAGGCTGTCCGTGTCCCCGTAG ACCGACTTTATGGGCCTGTCCTCGAGCGGAGTGCCTCGGTCCTCTTCGTAGAGGAACCCAGCCCACTCTGATACAAA AGCGCGTGTCCAGGCCAGCACAAAGGAGGCCACGTGGGAGGGGTAGCGGTCGTTGTCAACCAGGGGGTCCACCTTCT CTACGGTATGTAAACACATGTCCCCCTCCTCCACATCCAAGAATGTGATTGGCTTGTAAGTGTAGGCCACGTGACCA GGGGTCCCCGCCGGGGGGGTATAAAAGGGGGGGGCCTCTGTTCGTCCTCACTGTCTTCCGGATCGCTGTCCAGGAG CGCCAGCTGTTGGGGTAGGTATTCCCTCTCGAAGGCGGGCATGACCTCTGCACTCAGGTTGTCAGTTTCTAGGAACG AGGAGGATTTGATATTGACAGTACCAGCAGAGATGCCTTTCATAAGACTCTCGTCCATCTGGTCAGAAAACACAATC TTCTTGTTGTCCAGCTTGGTGGCAAATGATCCATAGAGGGCATTGGATAGAAGCTTGGCGATGGAGCGCATGGTTTG GTTCTTTTCCTTGTCCGCGCGCTCCTTGGCGGTGATGTTAAGCTGGACGTACTCGCGCGCCACACATTTCCATTCAG GAAAGATGGTTGTCAGTTCATCCGGAACTATTCTGATTCGCCATCCCCTATTGTGCAGGGTTATCAGATCCACACTG GTGGCCACCTCGCCTCGGAGGGGCTCATTGGTCCAGCAGAGTCGACCTCCTTTTCTTGAACAGAAAGGGGGGAGGGG GTCTAGCATGAACTCATCAGGGGGGTCCGCATCTATGGTAAATATTCCCGGTAGCAAATCTTTGTCAAAATAGCTGA TGGTGGCGGGATCATCCAAGGTCATCTGCCATTCTCGAACTGCCAGCGCGCGCTCATAGGGGTTAAGAGGGGTGCCC CAGGGCATGGGGTGGGTGAGCGCGGAGGCATACATGCCACAGATATCGTAGACATAGAGGGGCTCTTCGAGGATGCC GATGTAAGTGGGATAACATCGCCCCCCTCTGATGCTTGCTCGCACATAGTCATAGAGTTCATGTGAGGGGGCAAGAA GACCCGGGCCCAGATTGGTGCGGTTGGGTTTTTCCGCCCTGTAAACGATCTGGCGAAAGATGGCATGGGAATTGGAA GAGATAGTAGGTCTCTGGAATATGTTAAAATGGGCATGAGGTAAGCCTACAGAGTCCCTTATGAAGTGGGCATATGA CTCTTGCAGCTTGGCTACCAGCTCGGCGGTGATGAGTACATCCAGGGCACAGTAGTCGAGAGTTTCCTGGATGATGT CATAACGCGGTTGGCTTTTCTTTTCCCACAGCTCGCGGTTGAGAAGGTATTCTTCGTGATCCTTCCAGTACTCTTCG AGGGGAAACCCGTCTTTTTCTGCACGGTAAGAGCCCAACATGTAGAACTGATTGACTGCCTTGTAGGGACAGCATCC CTTCTCCACTGGGAGAGAGTATGCTTGGGCTGCATTGCGCAGCGAGGTATGAGTGAGGGCAAAAGTGTCCCTGACCA TGACTTTGAGGAATTGATACTTGAAGTCGATGTCATCACAGGCCCCCTGTTCCCAGAGTTGGAAGTCCACCCGCTTC TTGTAGGCGGGGTTGGGCAAAGCGAAAGTAACATCATTGAAGAGGATCTTGCCGGCCCTGGGCATGAAATTTCGGGT GATTTTGAAAGGCTGAGGAACCTCTGCTCGGTTATTGATAACCTGAGCGGCCAAGACGATCTCATCAAAGCCATTGA TGTTGTGCCCCACTATGTACAGTTCTAAGAATCGAGGGGTGCCCCTGACATGAGGCAGCTTCTTGAGTTCTTCAAAA GTGAGATCTGTAGGGTCAGTGAGAGCATAGTGTTCGAGGGCCCATTCGTGCACGTGAGGGTTCGCTTTAAGGAAGGA GGACCAGAGGTCCACTGCCAGTGCTGTTTGTAACTGGTCCCGGTACTGACGAAAATGCTGTCCGACTGCCATCTTTT CTGGGGTGACGCAATAGAAGGTTTGGGGGTCCTGCCGCCAGCGATCCCACTTGAGTTTTATGGCGAGGTCATAGGCG ATGTTGACGAGCCGCTGGTCTCCAGAGAGTTTCATGACCAGCATGAAGGGGATTAGCTGCTTGCCAAAGGACCCCAT CCAGGTGTAGGTTTCCACATCGTAGGTGAGAAAGAGCCTTTCTGTGCGAGGATGAGAGCCAATCGGGAAGAACTGGA TCTCCTGCCACCAGTTGGAGGAATGGCTGTTGATGTGATGGAAGTAGAACTCCCTGCGACGCGCCGAGCATTCATGC TTGTGCTTGTACAGACGGCCGCAGTACTCGCAGCGATTCACGGGATGCACCTTATGAATGAGTTGTACCTGACTTCC TTTGACGAGAAATTTCAGTGGAAAATTGAGGCCTGGCGCTTGTACCTCGCGCTTTACTATGTTGTCTGCATCGGCAT GACCATCTTCTGTCTCGATGGTGGTCATGCTGACGAGCCCTCGCGGGAGGCAAGTCCAGACCTCGGCGCGGCAGGGG CGGAGCTCGAGGACGAGAGCGCGCAGGCCGGAGCTGTCCAGGGTCCTGAGACGCTGCGGAGTCAGGTTAGTAGGCAG TGTCAGGAGATTAACTTGCATGATCTTTTGGAGGGCGTGAGGGAGGTTCAGATAGTACTTGATCTCAACGGGTCCGT TGGTGGAGATGTCGATGGCTTGCAGGGTTCCGTGTCCCTTGGGCGCTACCACCGTGCCCTTGTTTTTCATTTTGGAC GGCGGTGGCTCTGTTGCTTCTTGCATGTTTAGAAGCGGTGTCGAGGGCGCGCACCGGGCGGCAGGGGCGGCTCGGGA CCCGGCGGCATGGCTGGCAGTGGTACGTCGGCGCCGCGCGCGGGTAGGTTCTGGTACTGCGCCCTGAGAAGACTCGC ATGCGCGACGACGCGGCGGTTGACATCCTGGATCTGACGCCTCTGGGTGAAAGCTACCGGCCCCGTGAGCTTGAACC TGAAAGAGAGTTCAACAGAATCAATCTCGGTATCGTTGACGGCGGCTTGCCTAAGGATTTCTTGCACGTCGCCAGAG TTGTCCTGGTAGGCGATCTCGGCCATGAACTGCTCGATCTCTTCCTCTTGAAGATCTCCGCGGCCCGCTCTCTCGAC GGTGGCCGCGAGGTCGTTGGAGATGCGCCCAATGAGTTGAGAGAATGCATTCATGCCCGCCTCGTTCCAGACGCGGC TGTAGACCACAGCCCCCACGGGATCTCTCGCGCGCATGACCACCTGGGCGAGGTTGAGCTCCACGTGGCGGGTGAAG ACCGCATAGTTGCATAGGCGCTGGAAAAGGTAGTTGAGTGTGGTGGCGATGTGCTCGGTGACGAAGAAATACATGAT CCATCGTCTCAGCGGCATCTCGCTGACATCGCCCAGCGCTTCCAAGCGCTCCATGGCCTCGTAGAAGTCCACGGCAA AGTTAAAAAACTGGGAGTTACGCGCGGACACGGTCAACTCCTCTTCCAGAAGACGGATAAGTTCGGCGATGGTGGTG CGCACCTCGCGCTCGAAAGCCCCTGGGATTTCTTCCTCAATCTCTTCTTCTTCCACTAACATCTCTTCCTCTTCAGG TGGGGCTGCAGGAGGAGGGGGAACGCGGCGACGCCGGCGGCGCACGGGCAGACGGTCGATGAATCTTTCAATGACCT CTCCGCGGCGGCGGCGCATGGTCTCGGTGACGGCACGACCGTTCTCCCTGGGTCTCAGAGTGAAGACGCCTCCGCGC ATCTCCCTGAAGTGGTGACTGGGAGGCTCTCCGTTGGGCAGGGACACCGCGCTGATTATGCATTTTATCAATTGCCC CGTAGGTACTCCGCGCAAGGACCTGATCGTCTCAAGATCCACGGGATCTGAAAACCTTTCGACGAAAGCGTCTAACC AGTCGCAATCGCAAGGTAGGCTGAGCACTGTTTCTTGCGGGGGGGGCGGCTAGACGCTCGGTCGGGGTTCTCTCTT TCTTCTCCTTCCTCCTCTTGGGAGGGTGAGACGATGCTGCTGGTGATGAAATTAAAATAGGCAGTTTTGAGACGGCG GATGGTGGCGAGGAGCACCAGGTCTTTGGGTCCGGCTTGTTGGATACGCAGGCGATGAGCCATTCCCCAAGCATTAT CCTGACATCTGGCCAGATCTTTATAGTAGTCTTGCATGAGTCGTTCCACGGGCACTTCTTCTTCGCCCGCTCTGCCA TGCATGCGAGTGATCCCGAACCCGCGCATGGGCTGGACAAGTGCCAGGTCCGCTACAACCCTTTCGGCGAGGATGGC TTGCTGCACCTGGGTGAGGGTGGCTTGGAAGTCGTCAAAGTCCACGAAGCGGTGGTAGGCCCCGGTGTTGATTGTGT AGGAGCAGTTGGCCATGACTGACCAGTTGACTGTCTGGTGCCCAGGGCGCACGAGCTCGGTGTACTTGAGGCGCGAG TATGCGCGGGTGTCAAAGATGTAATCGTTGCAGGTGCGCACCAGGTACTGGTAGCCAATGAGAAAGTGTGGCGGTGG CTGGCGGTACAGGGGCCATCGCTCTGTAGCCGGGGCTCCGGGGGCGAGGTCTTCCAGCATGAGGCGGTGGTAGCCGT AGATGTACCTGGACATCCAGGTGATACCGGAGGCGGTGGTGGATGCACGTGGGAACTCGCGCACGCGGTTCCAGATG TTGCGCAGCGGCATGAAGTAGTTCATGGTAGGCACGGTCTGGCCAGTGAGGCGCGCGCAGTCATTGACGCTCTGTAG ACACGGAGAAAACGAAAGCGATGAGCGGCTCGACTCCGTGGTCTGGGGGAACGTGAACGGGTTGGGTCGCGGTGTAC CCCGGTTCGAGTCCAAAGCTAAGCGATCACGCTCGGATCGGCCGGAGCCGCGGCTAACGTGGTATTGGCTATCCCGT CTCGACCCAGCCGACGAATATCCAGGGTACGGAGTAGAGTCGTTTTTGCTGCTTTTTCCTGGACGTGTGCCATTGCC ACGTCAAGCTTTAGAACGCTCAGTTCTCGGGCCGTGAGTGGCTCGCGCCCGTAGTCTGGAGAATCAGTCGCCAGGGT TGCGTTGCGGTATGCCCCGGTTGGAGCCTAAGCGCGGCTCGTATCGGCCGGTTTCCGCGACAAGCGAGGGTTTGGCA GCCCCGTTATTTCCAAGACCCCGCCAGCCGACTTCTCCAGTTTACGGGAGCGAGCCCTTTTTTTTTTTTTTTTGTTT TTGTCGCCCAGATGCATCCAGTGCTGCGACAGATGCGCCCCCAGCAACAGGCCCCTTCTCAGCAACAGCCACAAAAG GCTCTTCTTGCTCCTGCAACTACTGCAGCTGCAGCCGTGAGCGGCGCGGGACAGCCCGCCTATGATCTGGACTTGGA AGAGGGCGAGGGATTGGCGCGCCTGGGGGCTCCATCGCCCGAGCGGCACCCGCGGGTGCAACTAAAAAAGGACTCTC GCGAGGCGTACGTGCCCCAGCAGAACCTGTTCAGGGACAGGAGCGGCGAGGAGCCAGAGGAGATGCGAGCATCTCGA TTTAACGCGGGTCGCGAGCTGCGCCACGGTCTGGATCGAAGACGGGTGCTGCAAGACGAGGATTTTGAGGTCGATGA AGTCACAGGGATCAGCCCAGCTAGGGCACATGTGGCCGCGGCCAACCTAGTCTCGGCCTACGAGCAGACCGTGAAGG AGGAGCGCAACTTCCAAAAATCTTTTAACAACCATGTGCGCACCCTGATCGCCCGCGAGGAAGTGACCCTGGGTCTG ATGCATCTGTGGGACCTGATGGAGGCTATCGCCCAAAACCCCACTAGCAAACCACTGACAGCTCAGCTGTTTCTGGT GGTTCAACATAGCAGGGACAACGAGGCATTCAGGGAGGCGTTGTTGAACATCACCGAGCCTGATGGGAGATGGCTGT ATGATCTGATCAACATCCTGCAAAGTATTATAGTGCAGGAACGTAGCCTGGGTTTGGCTGAGAAAGTGGCAGCTATC AACTACTCGGTCTTGAGCCTGGGCAAATACTACGCTCGCAAGATCTACAAGACCCCCTACGTACCCATAGACAAGGA GGTGAAGATAGATGGGTTTTACATGCGCATGACTCTGAAGGTGCTGACTCTGAGCGACGATCTGGGGGTGTATCGCA ATGACAGGATGCACCGCGCGGTGAGCGCCAGCAGGAGGCGCGAGCTGAGCGACAGAGAACTTATGCACAGCTTGCAA AGAGCTCTAACGGGGGCCGGGACTGATGGGGAGAACTACTTTGACATGGGAGCGGATTTGCAATGGCAACCCAGTCG CAGGGCCATGGAGGCTGCAGGGTGTGAGCTTCCTTACATAGAAGAGGTGGATGAAGTCGAGGACGAGGAGGGCGAGT ACTTGGAAGACTGATGGCGCGACCCGTATTTTTGCTAGATGGAACAGCAGCAGGCACCGGACCCCGCAATGCGGGCG GCGCTGCAGAGCCAGCCGTCCGGCATTAACTCCTCGGACGATTGGACCCAGGCCATGCAACGCATAATGGCGCTGAC GACCCGCAACCCCGAAGCCTTTAGACAGCAACCCCAGGCCAACCGCCTTTCTGCCATACTGGAGGCCGTAGTGCCCT CCCGCTCCAACCCCACCCACGAGAAGGTCCTGGCTATCGTGAACGCGCTGGTGGAGAACAAGGCCATCCGTCCCGAT GAGGCCGGGCTGGTATACAATGCTCTCTTGGAGCGCGTGGCCCGTTACAACAGCAGCAACGTGCAAACCAACCTGGA CCGGATGGTGACCGATGTGCGCGAGGCCGTGTCTCAGCGCGAGCGATTCCAGCGCGACGCCAACTTGGGGTCGTTGG TAGCGCTAAACGCTTTCCTCAGCACCCAGCCCGCCAACGTGCCCCGTGGTCAGCAAGACTATACAAACTTTTTGAGT GCATTGAGACTCATGGTAGCTGAGGTGCCCCAGAGCGAGGTGTACCAGTCCGGGCCAGATTACTTCTTCCAGACCAG CAGACAGGGCTTGCAGACAGTGAACCTGACTCAGGCTTTCAAGAACCTGAAGGGTCTGTGGGGAGTGCACGCCCCAG TAGGGGATCGCGCGACCGTGTCTAGCTTGCTGACTCCCAACTCCCGCCTGCTGCTGCTGCTGGTATCCCCCTTTACT GACAGCGGTAGCATTGACCGCAACTCGTACTTGGGCTACCTGCTTAACCTGTATCGCGAGGCCATAGGACAGAGCCA GGTGGACGAGCAGACCTATCAAGAAATCACCCAAGTGAGCCGCGCCCTGGGTCAGGAAGACACGGGCAGTTTGGAAG CCACCCTGAACTTCTTGCTAACCAACCGGTCACAGAAGATCCCTCCTCAGTATGCGCTTACCGCTGAGGAGGAGCGG ATCCTCAGATACGTGCAACAGAGCGTTGGACTGTTCCTGATGCAGGAGGGGGCGACACCTACCGCCGCGCTGGACAT GACAGCTCGAAACATGGAGCCCAGCATGTATGCTAGTAACAGGCCTTTCATTAACAAACTGCTGGACTACCTGCACA GGGCGGCCGCCATGAACTCTGATTATTTCACCAATGCTATCCTGAACCCACACTGGCTGCCCCCACCTGGTTTCTAC ACTGGCGAGTACGACATGCCCGACCCCAATGACGGGTTCCTGTGGGACGATGTGGACAGCAGCATATTTTCCCCGCC TCCCGGTTATACAGTTTGGAAGAAGGAAGGGGGCGATAGAAGACACTCTTCCGTGTCGCTATCCGGAACGGCTGGTG CTGCCGCGACCGTGCCCGAAGCTGCAAGTCCTTTCCCTAGCTTGCCCTTTTCACTAAACAGCGTTCGCAGCAGTGAA CTGGGGAGAATAACCCGCCCGCGCTTGATGGGCGAGGATGAGTACTTGAATGACTCTTTGCTGAGGCCAGAGAGGGA AAAGAACTTCCCCAACAATGGAATAGAGAGTCTGGTGGATAAGATGAGTAGATGGAAGACCTATGCGCAGGATCACA GAGACGAGCCCAGGATCTTGGGGGCTACAAGCAGACCGATCCGTAGACGCCAGCGCCACGACAGGCAGATGGGTCTT GTGTGGGACGATGAGGACTCTGCCGATGATAGCAGCGTGTTGGACTTGGGTGGAAGAGGAGGGGGCAACCCGTTCGC TCATCTGCGTCCCAGATTCGGGCGCATGTTGTAAAAGTGAAAGTAAAATAAAAAGGCAACTCACCAAGGCCATGGCG ACCGAGCGTGCGTTCGTTCTTTTTTGTTATCTGTGTCTAGTACGATGAGGAGACGAGCCGTGCTAGGCGGAGCGGTG GTGTATCCGGAGGGTCCTCCTCCTTCTTACGAGAGCGTGATGCAGCAACAGGCGGCGATGATACAGCCCCCACTGGA GGCTCCCTTCGTACCCCCACGGTACCTGGCGCCTACGGAAGGGAGAAACAGCATTCGTTACTCGGAGCTGTCGCCCC TGTACGATACCACCAAGTTGTATCTGGTGGACAACAAGTCGGCGGACATCGCCTCCCTGAACTATCAGAACGACCAC AGCAACTTCCTGACCACGGTGGTGCAGAACAATGACTTTACCCCCACGGAGGCTAGCACCCAGACCATCAACTTTGA CGAGCGGTCGCGATGGGGCGGTCAGCTGAAGACCATCATGCACACCAACATGCCCAACGTGAACGAGTACATGTTCA GCAACAAGTTCAAGGCGAGGGTGATGGTGTCCAGAAAAGCTCCTGAAGGTGTTACAGTAAATGACACCTATGATCAT AAAGAGGATATCTTGAAGTATGAGTGGTTTGAGTTCATTTTACCAGAAGGCAACTTTTCAGCCACCATGACGATCGA CCTGATGAACAATGCCATCATTGACAACTACCTGGAAATTGGCAGACAGAATGGAGTGCTGGAAAGTGACATTGGTG TTAAGTTTGACACTAGAAATTTCAGGCTCGGGTGGGACCCCGAAACTAAGTTGATTATGCCAGGTGTCTACACTTAT GAGGCATTCCATCCTGACATTGTATTGCTGCCTGGTTGCGGGGTAGACTTTACTGAAAGCCGACTTAGCAACTTGCT TGGCATCAGGAAGAGACATCCATTCCAGGAGGGTTTCAAAATCATGTATGAAGATCTTGAAGGGGGTAATATTCCTG CCCTTTTGGATGTCACTGCCTATGAGGAAAGCAAAAAGGATACCACTACTGAAACAACCACACTGGCTGTTGCAGAG GAAACTAGTGAAGATGATGATATAACTAGAGGAGATACCTATATAACTGAAAAACAAAAACGTGAAGCTGCAGCTGC TGAAGTTAAAAAAGAGTTAAAGATCCAACCTCTAGAAAAAGACAGCAAGAGTAGAAGCTACAATGTCTTGGAAGACA AAATCAACACAGCCTACCGCAGTTGGTACCTGTCCTACAATTACGGTAACCCTGAGAAAGGAATAAGGTCTTGGACA CTGCTCACCACTTCAGATGTCACCTGTGGGGCAGAGCAGGTCTACTGGTCGCTCCCTGACATGATGCAAGACCCAGT CACCTTCCGCTCCACAAGACAAGTCAACAACTACCCAGTGGTGGGTGCAGAGCTTATGCCCGTCTTCTCAAAGAGTT TCTACAATGAGCAAGCCGTGTACTCTCAGCAGCTCCGACAGGCCACTTCGCTCACGCACGTCTTCAACCGCTTCCCT GAGAACCAGATCCTCATCCGCCCGCCGGCGCCCACAATTACCACCGTCAGTGAAAACGTTCCTGCTCTCACAGATCA CGGGACCCTGCCGTTACGCAGCAGTATCCGGGGAGTCCAGCGCGTGACCGTTACTGACGCCAGACGCCGCACCTGTC CCTACGTTTACAAGGCCCTGGGCATAGTCGCGCCGCGCGTTCTTTCAAGCCGCACTTTCTAAAAAAAAAAAAAATGT CCATTCTCATCTCGCCCAGTAATAATACCGGTTGGGGACTGTATGCGCCCACCAAGATGTATGGAGGCGCCCGCAAG CGCTCTACCCAGCATCCTGTGCGCGTTCGCGGTCATTTCCGCGCTCCCTGGGGCGCACTCAAGGGTCGTACCCGCAC TCGGACCACGGTCGATGATGTGATCGACCAGGTGGTCGCCGATGCTCGTAATTATACTCCTACTGCGCCTACATCTA CTGTGGATGCAGTTATTGACAGTGTGGTGGCAGACGCCCGCGCCTATGCTCGCCGGAAGAGCCGAAGGAGGCGCATC GCCAGGCGCCACAGGGCTACTCCCGCCATGCGAGCCGCAAAAGCTATTCTGCGGAGGGCCAAACGTGTGGGGCGAAG AGCCATGCTTAGAGCGGCCAGACGCGCGGCTTCAGGTGCCAGCAGCGGCAGGTCCCGCAGGCGCGCGGCCACGGCGG CAGCAGCGGCCATTGCCAACATGGCCCAACCGCGAAGAGGCAATGTGTACTGGGTGCGTGATGCCACTACCGGCCAG CGCGTGCCCGTGCGCACCCGCCCCCCTCGCACTTAGAAGATACTGAGCAGTCTCCGATGTTGTGTCCCAGCGGCAAG TATGTCCAAGCGCAAATACAAGGAAGAGATGCTCCAGGTCATCGCGCCTGAAATCTACGGTCCGCCGGTGAAGGATG AAAAAAAGCCCCGCAAAATCAAGCGGGTCAAAAATAACAAAAAGGAAGAAGATGACGATGATGGGCTGGTGGAGTTT GTGCGCGAGTTCGCCCCAAGACGGCGCGTGCAGTGGCGCGGGCGCAAAGTGCGTCAAGTGCTCAGACCCGGGACCAC TGTGGTTTTTACACCTGGCGAGCGTTCCAGCACTACTTTTAAACGGTCCTATGATGAGGTGTACGGGGATGACGATA TTCTTGAGCAGGCGGCAGACCGCCTTGACGAGTTTGCTTATGGCAAGCGCACTAGATCCAGTCCCAAAGAGGAGGCT GTGTCCATTCCTTTGGATCATGGAAATCCCACCCCCAGCCTCAAACCAGTCACCCTGCAGCAAGTGCTGCCCGTGCC TGCGCGGAGAGGCGTAAAGCGCGAGGGTGAGGACCTATATCCCACCATGCAGCTAATGGTGCCCAAGCGCCAGAGGC TAGAAGACGTACTGGAGAAAATGAAAGTGGATGCCGATATCCAGCCTGAGGTCAAAGTAAGACCTATCAAGGAAGTG GCGCCAGGTTTGGGAGTACAAACCTTCGACATCAAGATTCCCACCGAGTCCATGGAAGTGCAGACCGAACCTGCAAA ACCCACCACCTCAATTGAGGTGCAAACGGAACCCTGGATGCCCGCGCCCGTTGCCGCCCCCAGCACCACTCGAAGAT CACGACGAAAGTACGGCCCAGCAAGTCTGCTAATGCCCAACTATGCTCTGCACCCATCCATCATTCCCACTCCGGGT TACAGAGGCACTCGCTACTATCGAAACCGGAGCAACACCTCTCGCCGCCGCAAACCACCTGCAAGTCGCACTCGCCG TCGCCGCCGCCGCAACACTGCCAGCAAATTGACTCCCGCCGCCCTGGTGCGGAGAGTGTACCGCGATGGTCGCGCTG AACCTCTGACGCTGCCGCGCGCGCGCTACCATCCAAGCATCACCACTTAATGACTGTTGACGCTGCCTCCTTGCAGA TATGGCCCTCACTTGCCGCCTTCGCGTCCCCATTACTGGCTACCGAGGAAGAAACTCGCGCCGTAGAAGGATGTTGG GGCGAGGGATGCGCCGCCACAGACGAAGGCGCGCTATCAGCAGACGATTAGGGGGTGGCTTTTTGCCAGCTCTTATA CCCATCATCGCCGCAGCGATCGGGGCGATACCAGGCATAGCTTCAGTGGCGGTTCAGGCCTCGCAGCGCCACTAACA TTGGAAAAAACTTATAAATAAAAAATAGAATGGACTCTGACGCTCCTGGTCCTGTGACTATGTTTTTGTAGAGATGG AAGACATCAATTTTTCATCCCTGGCTCCGCGACACGGCACGAGGCCGTACATGGGCACCTGGAGCGACATCGGCACG AGCCAACTGAACGGGGGCGCCTTCAATTGGAGCAGTATCTGGAGCGGGCTTAAAAATTTTGGCTCGACCGTAAAAAC CTATGGGAACAAAGCTTGGAACAGCAGCACAGGGCAGGCTCTGAGAAATAAGCTTAAGGAACAAAACTTCCAACAGA AGGTGGTCGATGGGATCGCCTCTGGTATTAACGGCGTAGTGGATCTGGCCAACCAGGCTGTACAAAAACAGATAAAC AGCCGCCTGGACCCGCCGCCCGCAACCCCTGGTGAAATGGAAGTGGAGGAAGAACTTCCTCCGCTGGAAAAGCGGGG CGACAAGCGTCCGCGACCCGAGCTAGAGCAGACGCTGGTGACGCGCGCAGACGAGCCCCCTTCATATGAGGAGGCAG TAAAGCTCGGAATGCCCACTACCAGGCCTGTAGCTCACATGGCTACCGGGGTGATGAAACCTTCTCAGTCACATCGA CCCGCCACCTTGGACTTGCCTCCTCCCCCTGCTTCTGCGGCGCCTGTTCCCAAACCTGTCGCTACCAGAAAGCCCAC CGCCGTACAGCCCGTCGCCGTAGCCAGACCGCGTCCTGGGGGCACACCGCGCCCGAAAGCAAACTGGCAAAGTACTC TGAACAGCATCGTGGGTCTGGGCGTGCAGAGTGTAAAGCGCCGTCGCTGCTATTAATTAAATATGGAGTAGCGCTTA ACTTGCTTGTCTGTGTGTATGTATCATCACCACGCCGCCGCAGCAGAGGAGAAAGGAAGAGGTCGCGCGCCGAGGCT GAGTTGCTTTCAAGATGGCCACCCCATCGATGATGCCCCAATGGGCATACATGCACATCGCCGGACAGGATGCTTCG GAGTACCTCAGTCCGGGTCTGGTGCAGTTCGCCCGTGCAACAGACACCTACTTCAGTATGGGGAACAAATTTAGAAA CCCCACAGTGGCGCCCACCCACGATGTGACCACCGACCGTAGCCAGCGCCTGATGCTGCGCTTCGTGCCCGTTGACC GGGAAGACAATACCTACTCTTACAAAGTTCGCTACACGCTGGCTGTAGGCGACAACAGAGTGCTTGACATGGCCAGC ACATTCTTTGACATTCGGGGGGTGCTTGATAGAGGTCCTAGCTTCAAGCCATATTCCGGCACAGCTTACAATTCACT CGCTCCTAAGGGCGCGCCCAATACATCTCAGTGGATAGTTACAACAAATGGGGACAATGCAGTAACTACCACCACAA ACACATTTGGCATTGCTTCCATGAAGGGAGACAATATTACTAAAGAAGGTTTGCAAATTGGGAAAGACATTACCACT ACTGAAGGAGAAGAAAAGCCCATTTATGCCGATAAAACATATCAGCCAGAGCCTCAAGTTGGAGAAGAATCATGGAC TGATACTGATGGAACAAATGAAAAGTTTGGTGGAAGAGCCCTTAAACCAGCTACCAACATGAAGCCATGCTACGGGT CTTTTGCAAGACCTACAAACATAAAAGGGGGCCAAGCTAAAAACAGAAAAGTAAAACCAACAACCGAAGGAGGGGTT GAAACTGAGGAACCAGATATTGATATGGAATTTTTCGATGGTAGAGATGCTGTTGCAGGAGCTTTAGCGCCTGAAAT TGTGCTTTATACGGAAAATGTAAATTTGGAAACTCCAGACAGTCATGTGGTATATAAACCAGAAACGTCTAATAACT CTCATGCAAATTTGGGTCAACAAGCCATGCCTAACAGACCCAATTACATTGGCTTCAGGGATAACTTCGTAGGCCTA ATGTACTACAACAGTACTGGAAATATGGGAGTTTTGGCTGGCCAAGCATCACAACTGAATGCAGTGGTTGACTTGCA GGACAGAAATACTGAACTGTCATATCAGCTTTTGCTTGATTCTCTGGGAGACAGAACCAGATACTTCAGCATGTGGA ATCAGGCTGTGGACAGTTACGATCCCGATGTTCGCATTATTGAAAATCATGGCATCGAGGATGAACTGCCTAATTAC TGTTTTCCTCTGAATGGCATAGGACCAGGGCACACATATCAAGGCATTAAAGTTAAAACCGATGACACTAATGGATG GGAAAAAGATGCTAATGTTGCTCCAGCTAATGAAATAACCATAGGCAACAACCTGGCTATGGAAATTAATATCCAAG CTAACCTTTGGAGAAGTTTTCTGTACTCTAATGTGGCTTTGTACCTTCCAGATGTTTACAAGTACACGCCACCTAAC ATTACTTTGCCCACTAACACCAACACCTATGAGTACATGAACGGGCGAGTGGTATCCCCATCCCTGGTTGATTCATA CATCAACATTGGCGCCAGGTGGTCTCTTGACCCAATGGACAATGTGAATCCATTCAACCACCACCGCAATGCTGGTC TGCGCTACAGGTCCATGCTTCTGGGAAATGGTCGTTATGTGCCTTTCCACATACAAGTGCCTCAGAAATTCTTTGCT GTCAAGAACCTACTTCTTCTACCTGGCTCCTACACCTACGAGTGGAACTTCCGAAAGGATGTGAACATGGTCCTGCA AAGTTCCCTTGGAAATGACCTCAGAACGGATGGTGCTACCATAAGTTTCACCAGCATCAATCTCTATGCCACCTTCT TCCCCATGGCTCACAACACAGCTTCCACCCTTGAAGCCATGCTGCGCAACGATACCAATGATCAGTCATTTAACGAC TACCTCTCTGCAGCTAACATGCTTTACCCCATTCCTGCCAATGCAACCAACATTCCAATTTCCATCCCATCTCGCAA CTGGGCAGCCTTCAGGGGCTGGTCCTTCACCAGACTCAAAACCAAGGAGACTCCATCTCTTGGATCAGGGTTCGATC CCTACTTCGTATATTCTGGATCTATTCCCTACCTGGATGGCACCTTTTACCTTAACCACACTTTCAAGAAGGTCTCC ATCATGTTTGACTCCTCAGTCAGCTGGCCTGGCAATGACAGGCTGTTGAGTCCAAATGAGTTTGAAATCAAGCGCAC TGTGGACGGGGAAGGATACAACGTGGCACAATGCAACATGACCAAAGACTGGTTCCTGGTTCAGATGCTTGCCAATT ACAACATTGGCTACCAGGGCTTTTACATCCCTGAGGGATACAAGGATCGCATGTACTCCTTTTTCAGAAACTTCCAG CCTATGAGCAGGCAGGTGGTTGATGAGGTTAATTACACTGACTACAAAGCCGTCACCTTACCATACCAACACAACAA CTCTGGCTTTGTAGGGTACCTTGCACCTACTATGAGACAAGGGGAACCTTACCCAGCCAATTATCCATACCCGCTCA TCGGAACTACTGCCGTTAAGAGTGTCACCCAGAAAAAGTTCCTGTGCGACAGGACCATGTGGCGCATTCCCTTCTCC AGCAACTTCATGTCCATGGGGGCCCTTACCGACCTGGGACAGAACATGCTCTATGCCAACTCAGCCCATGCGCTGGA CATGACTTTTGAGGTGGATCCCATGGATGAGCCCACCCTGCTTTATCTTCTTTTCGAAGTCTTCGACGTGGTCAGAG TGCACCAGCCACACCGCGGCGTCATCGAGGCCGTCTACCTGCGCACACCGTTCTCGGCCGGCAACGCCACCACATAA GAAGCCTCTTGCTTCTTGCAAGCAGCAGCTGCAGCCATGACATGCGGGTCCGGAAACGGCTCCAGCGAGCAAGAGCT CAAAGCCATCGTCCGAGACCTGGGCTGCGGACCCTATTTCCTGGGAACCTTTGACAAGCGTTTCCCGGGGTTCATGG CCCCCGACAAGCTCGCCTGCGCCATAGTCAACACTGCCGGACGCGAGACGGGGGGAGAGCACTGGCTGGCTTTTGGT TGGAACCCGCGCTACAACACCTGCTACCTTTTTGATCCTTTTGGGTTCTCGGATGAGCGGCTCAAACAGATTTACCA GTTTGAGTACGAGGGGCTCCTGCGTCGCAGTGCCCTTGCTACCAAAGACCGCTGCATCACCCTGGAGAAGTCTACCC AAAGCGTGCAGGGTCCGCGCTCAGCCGCCTGTGGACTTTTTTGCTGTATGTTCCTTCATGCCTTTGTGCACTGGCCC GACCGCCCCATGGACGGAAACCCCACCATGAAGTTGCTGACTGGGGTGTCCAACAGCATGCTCCAATCACCCCAAGT CCAGCCCACCCTGCGCCGCAACCAGGAGGTGCTATACCGCTTCCTAAACACCCACTCATCTTACTTTCGTTCTCACC GCGCGCGCATTGAAAGGGCCACCGCGTTTGACCGTATGGATATGCAATAAGTCATGTAAAACCGTGTTCAATAAACA GCACTTTATTTTTACATGCACTGAGGCTCTGGTTTTGCTCATTTGTTTCATCATTTACTCAGAAGTCGAATGGGTTC TGGCGGGAGTCAGAGTGACCCGCGGGCAGGGATACGTTGCGGAACTGTAACCTGTTCTGCCACTTGAACTCGGGGAT TACCAGCTTGGGAACTGGAATCTCGGGAAAGGTGTCTTGCCACAACTTTCTGGTCAGTTGCATAGCGCCAAGCAGGT CAGGAGCAGAGATCTTGAAATCACAGTTGGGGCCGGCATTCTGGACACGGGAGTTGCGATACACTGGGTTGCAACAC TGGAACACTATCAACGCTGGGTGTCTTACGCTTGCCAACACGGTTGGGTCACTGATGGTAGTCACATCCAAGTCTTC AGCATTGGCCATCCCAAAGGGGGTCATCTTACATGTCTGCCTGCCCATCACGGGAGCGCAGCCTGGCTTGTGGTTGC AATCACAATGAATGGGGATCAGCATCATCCTGGCTTGGTCGGGAGTTATCCCTGGGTACACAGCCTTCATGAAGGCT TCGTACTGCTTAAAAGCTTCCTGGGCCTTACTTCCCTCGGTGTAGAACATCCCACAGGACTTGCTGGAAAATTGATT AGTAGTACAGTTGGCATCATTCACACAACAGCGGGCATCGTTGTTGGCCAACTGAACCACATTTCTGCCCCAGCGGT TTTGGGTGATCTTGGCTCTGTCTGGATTCTCCTTCATAGCGCGCTGCCCGTTCTCGCTCGCCACATCCATCTCGATA ATGTGGTCCTTCTGGATCATGATAGTGCCATGCAGGCATTTCACCTTGCCTTCATAATCGGTGCATCCATGAGCCCA CAGAGCGCACCCGGTGCACTCCCAATTATTGTGGGCGATCTCAGAATAATAATGTACCAATCCCTGCATGAATCTTC CCATCATTGTTGTCAAGGTCTTCATGCTGGTAAATGTCAGCGGGATGCCACGGTGCTCCTCGTTCACATACTGGTGG CAGATACGCTTGTATTGCTCGTGCTGCTCTGGCATCAGCTTGAAAGAGGTTCTCAGATCATTATCCAGCCTGTACCT TTCCATTAGCACAGCCATCACTTCCATGCCCTTCTCCCAGGCAGATACCAGGGGCAGACTCAAAGGATTCCTAACAG CAATAAAAGTAGCTCCTTTAGCTATAGGGTCATTCTTGTCGATCTTCTCAACACTTCTCTTGCCATCCTTCTCAATG ATGCGCACCGGGGGGTAGCTGAAGCCCACGGCCACCAACTGAGCCTGTTCTCTTTCTTCTTCACTATCCTGGCTGAT GTCTTGCAGAGGGACATGCTTGGTCTTCCTGGGCTTCTTCTTGGGAGGGATCGGGGGAGGACTGTTGCTCCGCTCCG GAGACAGGGATGACTGCGAAGTTTCGCTTACCAATACCACCTGGCTCTCGGTAGAAGAACCGGACCCCACACGACGG TAGGTGTTCCTCTTCGGGGGCAGAGGTGGAGGCGACTGAGATGGGCTGCGGTCTGGCCTTGGAGGCGGATGGCTGGC AGAGCTCATTCCGCGTTCGGGGGTGTGCTCCCGGTGGCGGTCGCTTGACTGATTTCCTCCGCGGCTGGCCATTGTGT TCTCCTAGGCAGAGAAACAACAGACATGGAAACTCAGCCATCACTGCCAACATCGCTGCAAGCACCATCACACCTCG CCCCCAGCAGCGACGAGGAGGAGAGCTTAACCACCCCACCACCCAGTCCCGCTACCACCACCTCTACCCTCGATGAT GAGGAGGAGGTCGACGCAGCCCAGGAGATGCAGGCGCAGGATAATGTGAAAACGGAAGAGATTGAGGCAGATGTCGA GCAGGACCCGGGCTATGTGACGCCGGCGGAGCACCAGGAGGAGCTGAAACGCTTTCTAGACAGAGAGGATGACGACC GCCCAGAGCATCAAGCAGATGGCGTTTACCAGGAGGCTGGGATCAGGGATCATGTCGCCGACTACCTCACCGGCCTT GGTGGGGAGGACGTGCTCCTCAAACATCTAGCAAGGCAGTCGATCATAGTTAAAGACGCATTGCTCGATCTCACTGA AGTGCCCATCAGTGTGGAAGAGCTTAGCCGCGCCTACGAGCTGAACCTCTTTTCGCCTCAGGTACCCCCCAAGCGGC AGCCAAACGGCACCTGCGAGGCCAACCCTCGACTCAACTTCTATCCAGCTTTTACTATCCCCGAAGTGTTGGCCACC TACCACATCTTTTTCAAGAACCAAAAGATTCCAGTCTCCTGCCGCGCCAACCGCACCCGCGCCGATGCCCTGCTCAA CTTGGGTCCGGGAGCTCGCTTACCTGATATAGCTTCCTTGGAAGAGGTTCCAAAGATCTTTGAGGGTCTGGGAAGTG ATGAGACACGGGCCGCAAATGCTCTGCAACAGGGAGAGAATGACATGGATGAACACCACAGCGCTCTGGTGGAACTG GAGGGTGACAATGCCCGGATTGCAGTGCTCAAGCGCAGTATCGTGGTCACCCATTTTGCCTACCCCGCTGTTAACCT GCCCCCCAAAGTTATGAGCGCTGTCATGGACCATCTGCTCATCAAACGAGCAAGACCTCTTTCAGAAAACCAGAACA TGCAGGATCCAGACGCCTCGGACGAGGGCAAGCCGGTAGTCAGTGACGAGCAGCTATCTCGCTGGCTGGGTACCAAC TCCCCCCGAGATTTGGAAGAGAGGCGCAAGCTTATGATGGCTGTAGTGCTAGTAACTGTGGAGCTGGAGTGTCTGCG CCGCTTTTTCACCGACCCTGAGACCCTGCGCAAGCTAGAGGAGAACCTGCACTACACCTTTAGACATGGCTTCGTGC GGCAGGCATGCAAGATCTCCAACGTGGAGCTTACCAACCTGGTTTCTTACATGGGCATTTTGCATGAGAACCGACTA GGGCAGAGCGTCCTGCACACCACCCTTAAAGGGGAGGCCCGCCGTGACTACATCCGAGACTGTGTCTACCTCTACCT CTGCCATACCTGGCAAACTGGTATGGGTGTGTGGCAACAGTGTTTGGAAGAGCAGAACCTAAAAGAGCTGGACAAGC TCTTGCAGAGATCCCTCAAAGCCCTGTGGACAGGTTTTGATGAGCGCACCGTCGCCTCGGACCTGGCAGACATCATC TTCCCCGAGCGTCTCAGGGTTACTCTGCGAAACGGCCTGCCAGACTTTATGAGCCAGAGCATGCTTAACAACTTTCG CTCTTTCATCCTGGAACGCTCCGGTATCCTGCCTGCCACCTGCTGTGCGCTGCCCTCCGACTTTGTGCCTCTCACCT ACCGCGAGTGCCCACCGCCGCTATGGAGCCACTGCTACCTGTTCCGCCTGGCCAACTACCTCTCCTACCACTCGGAT GTTATAGAGGATGTGAGCGGAGACGGCCTGCTGGAATGCCACTGCCGCTGCAATCTTTGCACACCCCACCGCTCCCT TGCCTGCAACCCCCAGTTGCTGAGCGAGACCCAGATTATCGGCACCTTCGAGCTGCAGGGTCCCAGAAGTAAAGGCG AGGGGTCTTCTCCGGGGCAGAGTTTGAAACTGACACCGGGGCTGTGGACCTCCGCCTACCTGCGCAAGTTTCACCCC GAGGACTACCATCCCTATGAGATCAGGTTCTATGAGGACCAATCACATCCTCCCAAAGTCGAGCTCTCAGCCTGCGT CATCACCCAGGGAGCAATTCTGGCCCAATTGCAAGCCATCCAAAAATCTCGCCAAGAATTTCTGCTAAAAAAGGGAA ACGGGGTCTACCTTGACCCTCAGACCGGTGAGGAGCTCAACACAAGGTTCCCCCAGGATGTCCCATCGCCGAGGAAG CAAGAAGTTGAAGGTGCAGCTGTCGCCCCCAGAGGATATGAAGGAAGACTGGGACAGTCAGGCAGAGGAGGAGATGG AAGATTGGGACAGCCAGGCAGAGGAGGTGGACAGCCTGGAGGAAGACAGTTTGGAGGAGGAAGACGAGGAGGCAGAG GAGGTGGAAGAAGCAACCGCCGCCAAACAGTTGTCATCGGCGGCGGAGACAAGCAAGTCCCCAGACAGCAGCACGGC TACCATCTCCGCTCCGGGTCGGGGGGCCCAGCGGCGGCCCAACAGTAGATGGGACGAGACCGGGCGATTCCCAAACC CGACCACCGCTTCCAAGACCGGTAAGAAGGAGCGACAGGGATACAAGTCCTGGCGTGGACATAAAAACGCTATCATC TCCTGCTTGCATGAATGCGGGGGCAACATATCCTTCACCCGGCGATACCTGCTCTTCCACCACGGTGTAAACTTCCC CCGCAATATCTTGCATTACTACCGTCACCTCCACAGCCCCTACTGCAGTCAGCAAGTCCCGGCAACCCCGACAGAAA AATACAGCAGCGACAACGGTGACCAGAAAACCAGCAGTTAGAAAATCCACAACAAGTGCACCAGGAGGAGGACTGAG GATCACAGCGAACGAGCCAGCGCAGACCAGAGAGCTGAGGAACCGGATCTTTCCAACCCTCTATGCCATTTTCCAGC AGAGTCGGGGGCAAGAGCAGGAACTGAAAGTAAAAAACCGATCTCTGCGCTCGCTCACCAGAAGTTGTTTGTATCAC AAGAGCGAAGACCAACTTCAGCGCACTCTCGAGGACGCCGAGGCTCTCTTCAACAAGTACTGCGCGCTGACTCTTAA AGAGTAGCCCTTGCCCGCGCTCATTTTGAAAACGGCGGGAATCACGTCACCCTTGGCACCTGTCCTTTGCCCTTGTC ATGAGTAAAGAGATTCCCACGCCTTACATGTGGAGCTATCAGCCCCAAATGGGGTTGGCAGCAGGCGCTTCCCAGGA CTACTCCACCCGCATGAATTGGCTTAGCGCCGGGCCCTCAATGATATCACGGGTTAATGATATACGAGCTTATCGAA ACCAGTTACTCCTAGAACAGTCAGCTCTCACCACCACACCCCGTCAACACCTTAATCCCCGAAATTGGCCCGCCACC CTGGTGTACCAGGAAAATCCCGCTCCCACCACCGTACTACTTCCTCGAGACGCCCAGGCCGAAGTTCAGATGACTAA CGCAGGTGTACAGCTGGCGGGCGGTTCCGCCCTATGTCGTCACCGACCTCAACAGAGTATAAAACGCCTGGTGATTA GAGGCCGAGGTATCCAGCTCAACGACGAGTCGGTTAGCTCTTCGCTTGGTCTGCGACCAGACGGAGTCTTCCAAATC GCCGGCTGTGGGAGATCTTCCTTCACTCCTCGTCAGGCTGTGCTGACTTTGGAGAGTTCGTCCTCGCAGCCCCGCTC GGGCGGCATTGGAACTCTCCAGTTTGTGGAGGAGTTTACTCCCTCTGTCTACTTCAACCCCTTCTCCGGCTCTCCTG GCCAGTACCCGGACGAGTTCATACCAAACTTCGACGCAATCAGCGAGTCAGTGGATGGCTATGATTGATGTCTAATG GTGGTGCGGCTGAGCTAGCTCGACTGCGACACCTAGACCACTGCCGCCGCTTTCGCTGCTTCGCCCGGGAACTCACC GAGTTCATCTACTTCGAACTCTCCGAGGAGCACCCTCAGGGTCCGGCCCACGGAGTGCGGATTACCATCGAAGGGGG AATAGACTCTCGCCTGCATCGCATCTTCTCCCAGCGGCCCGTGCTAATTGAACGCGACCAGGGAAATACAACCATCT CCATCTACTGCATCTGTAACCACCCCGGATTGCATGAAAGCCTTTGCTGTCTTGTTTGTGCTGAGTTTAATAAAAAC TGAGTTAAGACCCTCCTACGGACTACCGCTTCTTCAATCAGGACTTTACAACACCAACCAGATCTTCCAGAAGACCC AGACCCTTCCTCCTTTCATCCAGGACTCTAACTCTACCTTACCAGCACCCTCCACTACTAACCTTCCCGAAACAAAC AAGCTTGCATCTCATCTGCAACACCGCCTTTCACGAAGCCTTCTTTCTGCCAATACTACCACTCCCAAAACCGGAGG TGAGCTCCGCGGTCTTCCTACTGACGACCCCTGGGTGGTAGCGGGTTTTGTAACGTTAGGAGTAGTTGCGGGTGGGC TTGTGCTGATCCTTTGCTACCTATACACACCTTGCTGTGCATATTTAGTCATATTGTGCTGTTGGTTTAAGAAATGG GGGCCATACTAGTCGTGCTTGCTTTACTTTCGCTTTTGGGTCTGGGCTCTGCTAATCTCAATCCTCTCGATCACGAT CCATGTTTAGACTTCGACCCAGAAAACTGCACACTTACTTTTGCACCCGACACAAGCCGTCTCTGTGGAGTTCTTAT TAAGTGCGGATGGGACTGCAGGTCCGTTGAAATTACACATAATAACAAAACATGGAACAATACCTTATCCACCACAT GGGAGCCAGGAGTTCCCCAGTGGTATACTGTCTCTGTCCGAGGTCCTGACGGTTCCATCCGCATTAGTAACAACACT TTCATTTTTTCTGAAATGTGCGATCTGGCCATGTTCATGAGCAGACAGTATGACCTATGGCCTCCCAGCAAAGAGAA CATTGTGGCATTTTCCATTGCTTATTGCTTGGTAACATGCATCATCACTGCTATCATTTGTGTGTGCATACACTTGC TTATAGTTATTCGCCCTAGACAAAGCAATGAGGAAAAAGAGAAAATGCCTTAACCTTTTTCCTCATACCTTTTCTTT ACAGCATGGCTTCTGTTACAGCTCTAATTATTGCCAGCATTGTCACTGTCGCTCACGGGCAAACAATTGTCCATATT ACCTTAGGACATAATCACACTCTTGTAGGGCCCCCAATTACTTCAGAGGTTATTTGGACCAAACTTGGAAGTGTTGA TTATTTTGATATAATTTGCAACAAAACTAAACCAATATTTGTAATCTGTAACAGACAAAATCTCACGTTAATTAATG TTAGCAAAATTTATAACGGTTACTATTATGGTTATGACAGATCCAGTAGTCAATATAAAAATTACTTAGTTCGCATA ACTCAGCCCAAATTAACAGTGCCAACTATGACAATAATTAAAATGGCTAATAAAGCATTAGAAAATTTTACATCACC AACAACACCCAATGAAAAAAACATTCCAAATTCAATGATTGCAATTATTGCGGCGGTGGCATTGGGAATGGCACTAA TAATAATATGCATGCTCCTATATGCTTGTTACTATAAAAAGTTTCAACATAAACAGGATCCACTACTAAATTTTAAC ATTTAATTTTTTATACAGATGATTTCCACTACAATTTTTATCATTACTAGCCTTGCAGCTGTAACTTATGGCCGTTC ACACCTAACTGTACCTGTTGGCTCAACATGTACACTACAAGGACCCCAAGAAGGCTATGTCACTTGGTGGAGAATAT ATGATAATGGAGGGTTCGCTAGACCATGTGATCAGCCTGGTACAAAATTTTCATGCAACGGAAGAGACTTGACCATT ATTAACATAACATCAAATGAGCAAGGCTTCTATTATGGAACCAACTATAAAAATAGTTTAGATTACAACATTATTGT AGTGCCAGCCACCACTTCTGCTCCCCGCAAATCCACTTTCTCTAGCAGCAGTGCCAAAGCAAGCACAATTCCTAAAA CAGCTTCTGCTATGTTAAAGCTTCCAAAAATCGCTTTAAGTAATTCCACAGCCGCTCCCAATACAATTCCTAAATCA ACAATTGGCATCATTACTGCCGTGGTAGTGGGATTAATGATTATATTTTTGTGTATAATGTACTACGCCTGCTGCTA TAGAAAACATGAACAAAAAGGTGATGCATTACTAAATTTTGATATTTAATTTTTTATAGAATTATGATATTGTTTCA ATCAAATACCACTACCTCCTATGCATACACAAACATTCAGCCTAAATACGCTATGCAACTAGAAATCACAATACTAA TTGTAATTGGAATTCTTATACTATCTGTTATTCTTTATTTTATATTCTGCCGTCAAATACCCAATGTTCATAGAAAT TCTAAAAGACGTCCCATCTATTCTCCTATGATTAGTCGTCCCCATATGGCTCTGAATGAAATCTAAGATCTTTTTTT TTTTCTCTTACAGTATGGTGAACATCAATCATGATCCCTAGAAATTTCTTCTTCACCATACTCATCTGTGCTTTTAA TGTCTGTGCTACTTTCACAGCAGTAGCCACTGCAAGCCCAGACTGTATAGGACCATTTGCTTCCTATGCACTTTTTG CCTTCGTTACTTGCATCTGCGTGTGTAGCATAGTCTGCCTGGTTATTAATTTTTTCCAACTGGTAGACTGGATCTTT GTGCGAATTGCCTACCTACGTCACCATCCCGAATACCGCAATCAAAATGTTGCGGCACTTCTTAGGCTTATTTAAAA CCATGCAGGCTATGCTACCAGTCATTTTAATTTTGCTACTACCCTGCATTCCCCTAGCTTCCACCGCCACTCGCGCT ACACCTGAACAACTTAGAAAATGCAAATTTCAACAACCATGGTCATTTCTTGATTGCTACCATGAAAAATCTGATTT TCCCACATACTGGATAGTGATTGTTGGAATAATTAACATACTTTCATGTACCTTTTTCTCAATCACAATATACCCCA CATTTAATTTTGGGTGGAATTCTCCCAATGCACTGGGTTACCCACAAGAACCAGATGAACATATTCCACTACAACAC ATACAACAACCACTAGCACTGGTACAGTATGAAAATGAGCCACAACCTTCACTGCCCCCTGCCATTAGTTACTTCAA CCTAACCGGCGGAGATGACTGACCCAATCGCCACATCATCCACCGCTGCCAAGGAGCTGCTGGACATGGACGGACGT GCCTCAGAACAGCGACTCATCCAACTACGCATTCGTCAGCAGCAGGAACGAGCAGTAAAAGAGCTAAGGGATGCCAT TGGGATTCACCAGTGCAAAAAAGGCATATTCTGCTTAGTAAAACAATCCAAAATCTCCTACGAGATCACCGCTACTG ACCATCGTCTCTCATACGAGCTCGGTCCGCAGCGACAAAAATTCACCTGCATGGTGGGAATCAACCCCATAGTTATC ACCCAGCAGTCTGGAGATACTAAGGGTTGTATCCAGTGTTCCTGTGATTCCACCGAGTGCATCTACACACTGCTGAA GACCCTCTGCGGCCTTCGAGACCTCCTACCCATGAACTAATCATTGCCCCTACCTTACCCAATCAAAATATTAATAA AGACACTTACTTGAAATCAGCAATACAGTCTTTGTCAAAACTTTCTACCAGCAGCACCTCACCCTCTTCCCAACTCT GGTACTCTAAACGTCGGAGGGTGGCATACTTTCTCCACACTTTGAAAGGGATGTCAAATTTTATTTCCTCTTCTTTG CCCACAATCTTCATTTCTTTATCCCCAGATGGCCAAGCGAGCTCGGCTAAGCACTTCCTTCAACCCGGTGTACCCTT ATGAAGATGAAAGCAGCTCACAACACCCATTTATAAATCCTGGTTTCATTTCCCCTGACGGGTTCACACAAAGTCCA AACGGGGTTTTAAGTCTTAAATGTGTTAATCCACTTACCACTGCAAGCGGCTCCCTCCAACTTAAAGTGGGAAGTGG TCTTACAGTAGACACTACTGATGGATCCTTAGAAGAAAACATCAAAGTTAACACCCCCCTAACAAAGTCAAACCATT CTATAAATTTACCAATAGGAAACGGTTTGCAAATAGAACAAAACAAACTTTGCAGTAAACTCGGAAATGGTCTTACA TTTGACTCTTCCAATTCTATTGCACTGAAAAATAACACTTTATGGACAGGTCCAAAACCAGAAGCCAACTGCATAAT TGAATACGGGAAACAAAACCCAGATAGCAAACTAACTTTAATCCTTGTAAAAAATGGAGGAATTGTTAATGGATATG TAACGCTAATGGGAGCCTCAGACTACGTTAACACCTTATTTAAAAACAAAAATGTCTCCATTAATGTAGAACTATAC TTTGATGCCACTGGTCATATATTACCAGACTCATCTTCTCTTAAAACAGATCTAGAACTAAAATACAAGCAAACCGC TGACTTTAGTGCAAGAGGTTTTATGCCAAGTACTACAGCGTATCCATTTGTCCTTCCTAATGCGGGAACACATAATG AAAATTATATTTTTGGTCAATGCTACTACAAAGCAAGCGATGGTGCCCTTTTTCCGTTGGAAGTTACTGTTATGCTT AATAAACGCCTGCCAGATAGTCGCACATCCTATGTTATGACTTTTTTATGGTCCTTGAATGCTGGTCTAGCTCCAGA AACTACTCAGGCAACCCTCATAACCTCCCCATTTACCTTTTCCTATATTAGAGAAGATGACTGACAACAAAAATAAA GTTCAACATTTTTTATTGAAATTCCTTTTACAGTATTCGAGTAGTTATTTTGCCTCCCCCTTCCCATTTAACAGAAT ACACCAATCTCTCCCCACGCACAGCTTTAAACATTTGGATACCATTAGAGATAGACATAGTTTTAGATTCCACATTC CAAACAGTTTCAGAGCGAGCCAATCTGGGGTCAGTAATACATAAAAATGCATCGGGATAGTCTTTTAAAGCGCTTTC ACAGTCCAACTGTTGCGGATGCGACTCCGGAGTCTGAATCACGGTCATCTGGAAGAAGAACGATGGGAATCATAATC CGAAAACGGAATCGGGCGATTGTGTCTCATCAAACCCACAAGCAACCGCTGTCTGCGTCGCTCCGTGCGACTGCTGT TTATGGGATCGGGGTCCGCAGTGTCCTGAAGCATGATTTTAATAGCCCTTAACATTAACTTTCTGGTGCGATGCGCG CAGCAACGCATTCTGATTTCACTTAGATTACTACAGTAGGTACAGCACATTATCACAATATTGTTTAATAAACCATA ATTAAAAGCGCTCCAGCCAAAACTCATATCTGATATAATCGCCCCTGCATGACCATCATACCAAAGTTTAATATAAA TTAAATGTCGTTCCCTCAAAAACACACTACCCACATACATGATCTCTTTTGGCATGTGCATATTAACAATTTGTCTG TACCATGGACAACGTTGGTTAATCATGCAACCCAATATAACCTTCCGGAACCACACTGCCAACACCGCTCCCCCAGC CATGCATTGAAGTGAACCCTGCTGATTACAATGACAATGAAGAACCCAATTCTCTCGACCATGAATCACTTGAGACT GAAAAATATCTATAGTAGCACAACAAAGACATAAATGCATGCATCTTCTCATAATTTTTAACTCCTCTGGATTTAAA AACATATCCCAAGGAATGGGAAACTCTTGCAGAACAGTAAAGCTGGCAGAACAAGGAAGACCACGAACACAACTTAC ACTATGCATAGTCATAGTATCACAATCTGGCAACAGCGGGTGGTCTTCAGTCATAGAAGCTCGGGTTTCATTTTCCT CACATCGTGGTAATTGGGCTCTGGTGTAAGGGTGATGTCTGGCGCATGATGTGGAGCGTGCGCGCAACCTTGTCATA ATGGAGTTGCTTCCTGACATTCTCGTATTTTGTATAGCAAAACGCTGCCCTGGCACAACACACTCTTCTTCGTCTTC TATCCTGCCGCTTAGTGTGTTCCGTCTGATAATTCAAGTACAGCCACACTCTTAAGTTGGTCAAAAGAATGCTGGCT TCAGTTGTAATCAAAACTCCATCATATTTAATTGTTCTAAGGAAATCATCCACGGTAGCATATGCAAATCCCAACCA AGCAATGCAACTGGATTGCGTTTCAAGCAGCAGAGGAGAGGGAAGAGACGGAAGAATCATGTTAATTTTTATTCCAA ACGATCTCGCAGTACTTCAAATTGTAGATCGCGCAGATGGCATCTATCGCCCCCACTGTGTTGGTGAAAAAGCACAG CTAAATCAAAAGAAATGCGATTTTCAAGGTGCTCAACGGTGGCTTCCAACAAAGCCTCCACGCGCACATCCAAAAAC AAAAGAATACCAAAAGAAGGAGCATTTTCTAACTCCTCAAACATCATATTACATTCCTGCACCATTCCCAGATAATT TTCAGCTTTCCAGCCTTGAATTATTCGTGTCAGTTCTTGTGGTAAATCCAAACCACACATTACAAACAGGTCCCGGA GGGCGCCCTCCACCACCATTCTTAAACACACCCTCATAATGACAAAATATCTTGCTCCTGTGTCACCTGTAGCAAAT TAAGAATGGCATCATCAATTGACATGCCCTTGGCTCTAAGTTCTTCTCTAAGTTCTAGTTGTAGATACTCTCTCATA TTATCACCAAACTGCTTAGCCAGAAGCCCCCCGGGAACAATAGCAGGGGACGCTACAGTGCAGTACAAGCGCAGACC TCCCCAATTGGCTCCAGCAAAAACAAGATTAGAATAAGCATACTGGGAACCACCAGTAATATCATCAAAGTTGCTGG AAATATAATCAGGCAGAGTTTCTTGTAAAAATTGAATAAAAGAAAAATTTTCCAAAGAAACATTCAAAACCGTTGGG ATGCAAATACAATAGGTTACCGCGCTGCGCTCCAACATTGTTAGTTTTGAATTAGTCTGCAAAATAAAAGAAACAAG CGTCATATCATAGTAGCCTGTCGAACAGGTGGAAAAATCAGTCTTTCCATCACAAGACAAGCCACAGGGTCTCCAGC TCGACCCTCGTAAAACCTGTCATTGTGATTAAACAACAGCACCGAAAGTTCCTCGCGGTGGCCAGCATGAATAATTC TTGATGAAGCATACAATCCAGACATGTTAGCATCAGTTAAAGAGAAAAAACAGCCAACATAGCCTCTGGGTATAATT ATGCTTAATTTTAAGTATAGCAAAGCCACCCCTCGCGGATACAAAGTAAAAGGCACAGGAGAATAAAAAATATAATT ATTTCTCTGCTGCTGTTCAGGCAACGTTGCTCCCGGTCCCTCTAAATAGACATACAAAGCCTCATCAGCCATGGCTT ACCAGGCAAAGTACAGCGGGCGCACAAAGCACAAGCTCTAAAGAAGCTCTAAAAACACTCTCCAACCTCTCCACAAT ATATACACAAGCCCTAAACTGACGTAATGGGAGTAAAGTGAAAAAAAAATACCGCCAAGCCCAACACACACCCCGAA ACTGCGTCAGCAGGAAAAAGTACAGTTTCACTTCCGCATTCCCAACAAGCGTAACTTCCTCTTTCTCATGGTACGTC ACATCCGATTAACTTGCAACGTCATTTTCCCACGGTCGCGCCGCCCCTTTTAGCCGTTAACCCCGCAGCCAATCACC ACACAGCGCGCACTTTTTTAAATTACCTCATTTACATGTTGGCACCATTCCATCTATAAGGTATATTATATAGATAG

GenBank Accession No. YP_002213796 MAKRARLSTSFNPVYPYEDESSSQHPFINPGFISPDGFTQSPNGVLSLKCVNPLTTASGSLQLKVGSGLTVDTTDGS LEENIKVNTPLTKSNHSINLPIGNGLQIEQNKLCSKLGNGLTFDSSNSIALKNNTLWTGPKPEANCIIEYGKONPDS KLTLILVKNGGIVNGYVTLMGASDYVNTLFKNKNVSINVELYFDATGHILPDSSSLKTDLELKYKQTADFSARGEMP STTAYPFVLPNAGTHNENYIFGQCYYKASDGALFPLEVTVMLNKRLPDSRTSYVMTFLWSLNAGLAPETTOATLITS PFTFSYIREDD

GenBank Accession No. YP_002213774 MRRRAVLGGAVVYPEGPPPSYESVMQQQAAMIOPPLEAPFVPPRYLAPTEGRNSIRYSELSPLYDTTKLYLVDNKSA DIASLNYQNDHSNFLTTVVONNDFTPTEASTQTINFDERSRWGGOLKTIMHTNMPNVNEYMFSNKFKARVMVSRKAP EGVTVNDTYDHKEDILKYEWFEFILPEGNFSATMTIDLMNNAIIDNYLEIGRONGVLESDIGVKEDTRNFRLGWDPE TKLIMPGVYTYEAFHPDIVLLPGCGVDFTESRLSNLLGIRKRHPFQEGFKIMYEDLEGGNIPALLDVTAYEESKKDT TTETTTLAVAEETSEDDDITRGDTYITEKQKREAAAAEVKKELKIQPLEKDSKSRSYNVLEDKINTAYRSWYLSYNY GNPEKGIRSWTLLTTSDVTCGAEQVYWSLPDMMQDPVTFRSTRQVNNYPVVGAELMPVFSKSFYNEQAVYSQQLRQA TSLTHVENRFPENQILIRPPAPTITTVSENVPALTDHGTLPLRSSIRGVQRVTVTDARRRTCPYVYKALGIVAPRVL SSRTF

GenBank Accession No. YP_002213779 MATPSMMPQWAYMHIAGQDASEYLSPGLVQFARATDTYFSMGNKERNPTVAPTHDVTTDRSQRLMLRFVPVDREDNT YSYKVRYTLAVGDNRVLDMASTFFDIRGVLDRGPSFKPYSGTAYNSLAPKGAPNTSQWIVTTNGDNAVTTTTNTFGI ASMKGDNITKEGLQIGKDITTTEGEEKPIYADKTYQPEPQVGEESWTDTDGTNEKFGGRALKPATNMKPCYGSFARP TNIKGGQAKNRKVKPTTEGGVETEEPDIDMEFFDGRDAVAGALAPEIVLYTENVNLETPDSHVVYKPETSNNSHANL GQQAMPNRPNYIGERDNFVGLMYYNSTGNMGVLAGQASQLNAVVDLODRNTELSYQLLLDSLGDRTRYFSMWNQAVD SYDPDVRIIENHGIEDELPNYCFPLNGIGPGHTYQGIKVKTDDINGWEKDANVAPANEITIGNNLAMEINIQANLWR SFLYSNVALYLPDVYKYTPPNITLPTNTNTYEYMNGRVVSPSLVDSYINIGARWSLDPMDNVN PFNHHRNAGLRYRS MLLGNGRYVPFHIQVPQKFFAVKNLLLLPGSYTYEWNFRKDVNMVLQSSLGNDLRTDGATISFTSINLYATFFPMAH NTASTLEAMLRNDTNDQSFNDYLSAANMLYPI PANATNIPISIPSRNWAAFRGWSFTRLKTKETPSLGSGFDPYFVY SGSIPYLDGTFYLNHTFKKVSIMFDSSVSWPGNDRLLSPNEFEIKRTVDGEGYNVAQCNMTKDWFLVOMLANYNIGY QGFYIPEGYKDRMYSFFRNFQPMSROVVDEVNYTDYKAVTLPYQHNNSGFVGYLAPTMRQGEPYPANYPYPLIGTTA VKSVTQKKFLCDRTMWRIPFSSNFMSMGALTDLGQNMLYANSAHALDMTFEVDPMDEPTLLYLLFEVFDVVRVHQPH RGVIEAVYLRTPFSAGNATT

GenBank Accession No. AC_000018 (SEQ ID NO: 210) CTCTCTATTTAATATACCTTATAGATGGAATGGTGCCAATATGTAAATGAGGTAATTTAAAAAAGTGCGCGCTGTGT GGTGATTGGCTGTGGGGTTAACGGCTAAAATGGGCGGGGCGGCCGTGGGAAAATGACGTGACTTATGTGGGAGGAGC TATGTTGCAAGTTATTGCGGTAAATGTGACGTAAAACGAGGTGTGGTTTGAACACGGAAGTAGACAGTTTTCCCACG CTTACTGACAGGATATGAGGTAGTTTTGGGCGGATGCAAGTAAAAATTCTCCATTTTCGCGCGAAAACTGAATGAGG AAGTGAATTTCTGAGTCATTTCGCGGTTATGACAGGGTGGAGTATTTGCCGAGGGCCGAGTAGACTTTGACCGTTTA CGTGGAGGTTTCGATTACCGTGTTTTTCACCTAAATTTCCGCGTACGGTGTCAAAGTCCTGTGTTTTTACGTAGGTG TCAGCTGATCGCTAGGGTATTTAAACCTGACGAGTTCCGTCAAGAGGCCACTCTTGAGTGCCAGCGAGAAGAGTTTT CTCCTTCGCGCCGCAAGTCAGTTCTGCGCTTTGAAAATGAGACACCTGCGTTTCCTGCCACAGGAGATTATCTTCAG TGAGACCGGGATCGAAATACTGGAGTTTGTGGTAAATACCCTAATGGGAGACGACCCGGAACCGCCAGTGCAGCCTT TCGATCCACCTACGCTGCACGATCTGTATGATTTAGAGGTAGACGGGCCTCAGGATCCCAATGAGGAAGCTGTGAAT GGGTTTTTTACTGATTCTATGCTGCTAGCTGCCGATGAAGGATTGGACATAAACCCTCCTCCTGAGACCCTTGTTAC CCCAGGGGTGGTTGTGGAAAGCGGCAGAGGTGGGAAAAAATTGCCTGATCTGGGAGCAGCTGAAATGGACTTGCGTT GTTATGAAGAGGGTTTTCCTCCGAGTGATGATGAAGATGGGGAAACTGAGCAGTCCATCCATACCGCAGTGAATGAG GGAGTAAAAGCTGCCAGCGATGTTTTTAAGTTGGACTGTCCGGAGCTGCCTGGACATGGCTGTAAGTCTTGTGAATT TCACAGGAATAACACTGGAATGAAAGAACTATTGTGCTCGCTTTGCTATATGAGAATGCACTGCCACTTTATTTACA GTAAGTGTATTTAAGTGAAATTTAAAGGAATAGTGTAGCTGTTTAATAACTGTTGAATGGTAGATTTATGTTTTTTA CTTGTGATTTTTTGTAGGTCCTGTGTCTGATGATGAGTCACCTTCTCCTGATTCAACTACCTCACCTCCTGAAATTC AGGCGCCCGCACCTGCAAACGTATGCAAGCCCATTCCTGTAAAGCCTAAGCCTGGGAAACGCCCTGCTGTGGATAAG CTTGAGGACTTGTTGGAGGGTGGGGATGGACCTTTGGACCTTAGTACCCGGAAACTGCCAAGGCAATAAGTGCCCTG CAGCTGTGTTTATTTAATGTGACGTCATGTAATAAAATTATGTCAGCTGCTGAGTGTTTTATTACTTCTTGGGTGGG GACTTGGATATATAAGTAGGAGCAGATCTGTGTGGTTAGCTCACAGCAACCTGCTGCCATCCATGGAGGTTTGGGCT ATCTTGGAAGACCTCAGACAGACTAGGCTACTGCTAGAAAACGCCTCGGACGGAGTCTCTGGCCTTTGGAGATTCTG GTTCGGTGGTGATCTAGCTAGGCTAGTGTTTAGGATAAAACAGGACTACAGCGTAGAATTTGAAAAGTTATTGGACG ACAGTCCAGGACTTTTTGAAGCTCTTAACTTGGGTCATCAGGCTCATTTTAAGGAGAAGGTTTTATCAGTTTTAGAT TTTTCTACTCCTGGTAGAACTGCTGCTGCTGTAGCTTTTCTTACTTTTATATTGGATAAATGGATCCGCCAAACTCA CTTCAGCAAGGGATACGTTTTGGATTTCATAGCAGCAGCTTTGTGGAGAACATGGAAGGCTCGCAGGATGAGGACAA TCTTAGATTACTGGCCAGTGCAGCCTCTAGGAGTAGCAGGGATACTGAGACACCCACCGACCATGCCAGCGGTTCTG CAGGAGGAGCAGCAGGAGGACAATCCGAGAGCCGGCCTGGACCCTCCGGTGGAGGAGTAGCTGACCTGTTTCCTGAA CTGCGACGGGTGCTTACTAGGTCTACGACCAGTGGACAGAACAGAGGCATTAAGAGGGAGAGGAATCCTAGTGGGAA TAATTCAAGAACCGAGTTGGCTTTAAGTTTAATGAGCCGCAGGCGTCCTGAAACTGTTTGGTGGCATGAGGTTCAGA GCGAAGGCAGGGATGAAGTTTCAATATTGCAGGAGAAATATTCACTAGAACAACTTAAGACCTGTTGGTTGGAACCT GAGGATGATTGGGAGGTGGCCATTAGGAATTATGCTAAGATATCTCTGAGGCCTGATAAACAATATAGAATTACTAA GAAGATTAATATTAGAAATGCATGCTACATATCAGGGAATGGGGCAGAGGTTATAATAGATACACAAGATAAAGCAG CTTTTAGATGTTGTATGATGGGTATGTGGCCAGGGGTTGTCGGCATGGAAGCAGTAACACTTATGAATATTAGGTTT AGAGGGGATGGGTATAATGGCATTGTATTTATGGCTAACACTAAGCTGATTCTACATGGTTGTAGCTTTTTTGGGTT TAATAATACGTGTGTAGAAGCTTGGGGGCAAGTTAGTGTGAGGGGTTGTAGTTTTTATGCATGCTGGATTGCAACAT CAGGTAGGGTCAAGAGTCAGTTGTCTGTGAAGAAATGCATGTTTGAGAGATGTAATCTTGGCATACTGAATGAAGGT GAAGCAAGGATCCGCCACTGCGCAGCTACAGAAACTGGCTGCTTCATTCTAATAAAGGGAAATGCCAGTGTGAAGCA TAATATGATCTGTGGACATTCGGATGAGAGGCCTTATCAGATGCTGACCTGCGCTGGTGGACATTGCAATATTCTTG CTACTGTGCATATCGTTTCACATGCACGCAAGAAATGGCCTGTATTTGAACATAATGTGATTACCAAGTGCACCATG CACATAGGTGGTCGCAGGGGAATGTTTATGCCTTACCAGTGTAACATGAATCATGTGAAGGTAATGTTGGAACCAGA TGCCTTTTCCAGAGTGAGCTTAACAGGAATCTTTGATATGAATATTCAACTATGGAAGATCCTGAGATATGATGACA CTAAACCGAGGGTGCGCGCATGCGAATGCGGAGGCAAGCATGCTAGATTCCAGCCGGTGTGCGTGGATGTGACTGAA GACCTGAGACCCGATCATTTGGTGCTTGCCTGCACTGGAGCGGAGTTCGGTTCCAGTGGTGAAGAAACTGACTAAAG TGAGTAGTGGGGGCAAGATGTGGATGGGGACTTTCAGGTTGGTAAGGTGGACAGATTGGGTAAATTTTGTTAATTTC TGTCTTGCAGCTGCCATGAGTGGAAGCGCTTCTTTTGAGGGGGGAGTATTTAGCCCTTATCTGACGGGCAGGCTCCC ACCATGGGCAGGAGTTCGTCAGAATGTCATGGGATCCACTGTGGATGGGAGACCCGTCCAGCCCGCCAATTCCTCAA CGCTGACCTATGCCACTTTGAGTTCGTCACCATTGGATGCAGCTGCAGCCGCCGCCGCTACTGCTGCCGCCAACACT ATCCTTGGAATGGGCTATTACGGAAGCATCGTTGCCAATTCCAGTTCCTCTAATAACCCTTCAACCCTGGCTGAGGA CAAGCTACTTGTTCTGTTGGCTCAGCTCGAGGCCTTAACCCAACGCTTAGGCGAACTGTCTAAGCAGGTGGCCCAGT TGCGTGAGCAAACTGAGTCTGCTGTTGCTACAGCAAAGTCTAAATAAAGATCTCAAATCAATAAATAAAGAAATACT TGTTATAAAAACAAATGAATGTTTATTTGATTTTTCGCGCGCGGTATGCCCTGGACCATCGGTCTCGATCATTGAGA ACTCGGTGGATCTTTTCCAGTACCCTGTAAAGGTGGGATTGAATGTTTAGATACATAGGCATTAGTCCGTCTCGGGG GTGGAGATAGCTCCATTGAAGAGCCTCTTGCTCCGGGGTAGTGTTATAAATCACCCAGTCATAGCAAGGTCGGAGTG CATGGTGTTGCACAATATCTTTTAGGAGCAGACTAATTGCAACGGGGAGGCCCTTAGTGTAGGTGTTTACAAATCTA TTGAGCTGGGACGGGTACATCCGGGGTGAAATTATATGCATTTTGGACTGGATCTTGAGGTTGGCAATGTTGCCGCC TAGATCCCGTCTCGGGTTCATATTGTGCAGGACCACTAAGACAGTGTATCCGGTGCACTTGGGAAATTTATCATGCA GCTTAGAGGGAAAAGCATGAAAAAATTTGGAGACGCCTTTGTGACCCCCCAGATTCTCCATGCACTCATCCATAATG ATAGCGATGGGGCCGTGGGCAGCGGCACGGGCGAACACGTTCCGGGGGTCTGAAACATCATAGTTATGCTCCTGAGT CAGGTCATCATAAGCCATTTTAATAAACTTTGGGCGGAGGGTGCCAGATTGGGGGATGAAAGTTCCCTCTGGCCCGG GAGCATAGTTCCCCTCACATATTTGCATTTCCCAGGCTTTCAGTTCAGAGGGGGGGATCATGTCCACCTGCGGGGCT ATAAAAAATACCGTTTCTGGAGCCGGGGTGATTAACTGGGATGAGAGCAAATTCCTAAGCAGCTGAGACTTGCCGCA CCCAGTGGGACCGTAAATGACCCCAATTACGGGTTGCAGATGGTAGTTTAGGGAGCGACAGCTGCCGTCCTCCCGGA GTAGGGGGGCCACTTCGTTCATCATTTCCCTTACATGGATATTTTCCCGCACCAAGTCCGTTAGGAGGCGCTCTCCC CCAAGGGATAGAAGCTCCTGGAGCGAGGAGAAGTTTTTCAGCGGCTTCAGCCCGTCAGCCATGGGCATTTTGGAAAG AGTCTGTTGCAAGAGCTCGAGCCGGTCCCAGAGCTCGGTGATGTGCTCTATGGCATCTCGATCCAGCAGACCTCCTC GTTTCGCGGGTTGGGACGGCTCCTGGAGTAGGGAATCAGACGATGGGCGTCCAGCGCTGCCAGGGTCCGATCCTTCC ATGGTCGCAGCGTCCGAGTCAGGGTTGTTTCCGTCACGGTGAATGGGTGCGCGCCTGGTTGGGCGCTTGCGAGGGTG CGCTTCAGACTCATCCTGCTGGTCGAGAACCGCTGCCGATCGGCGCCCTGCATGTCGGCCAGGTAGCAGTTTACCAT GAGTTCGTAGTTGAGCGCCTCGGCCGCGTGGCCTTTGGCACGGAGCTTACCTTTGGAAGTTTTATGGCAGGCGGGGC AGTAGATACATTTGAGGGCATACAGCTTGGGCGCGAGGAAAATGGATTCGGGGGAGTATGCATCCGCACCGCAGGAG GCGCAGACGGTTTCGCACTCCACGAGCCAGGTCAGATCCGGCTCATCGGGGTCAAAAACAAGTTTTCCGCCATGTTT TTTGATGCGTTTCTTACCTTTGGTTTCCATGAGGTCGTGTCCCCGCTGGGTGACAAAGAGGCTGTCCGTGTCCCCGT AGACCGACTTTATGGGCCTGTCCTCGAGCGGAGTGCCTCGGTCCTCTTCGTAGAGGAACCCAGCCCACTCTGATACA AAAGCGCGTGTCCAGGCCAGCACAAAGGAGGCCACGTGGGAGGGGTAGCGGTCGTTGTCAACCAGGGGGTCCACCTT CTCTACGGTATGTAAACACATGTCCCCCTCCTCCACATCCAAGAATGTGATTGGCTTGTAAGTGTAGGCCACGTGAC CAGGGGTCCCCGCCGGGGGGGTATAAAAGGGGGCGGGCCTCTGTTCGTCCTCACTGTCTTCCGGATCGCTGTCCAGG AGCGCCAGCTGTTGGGGTAGGTATTCCCTCTCGAAGGCGGGCATGACCTCTGCACTCAGGTTGTCAGTTTCTAGGAA CGAGGAGGATTTGATATTGACAGTACCAGCAGAGATGCCTTTCATAAGACTCTCGTCCATCTGGTCAGAAAACACAA TCTTCTTGTTGTCCAGCTTGGTAGCAAATGATCCATAGAGGGCATTGGATAGAAGCTTGGCGATGGAGCGCATGGTT TGGTTCTTTTCCTTGTCCGCGCGCTCCTTGGCGGCGATGTTAAGCTGGACGTACTCGCGCGCCACACATTTCCATTC AGGGAAGATGGTTGTCAGTTCATCCGGAACTATTCTGACTCGCCATCCCCTATTGTGCAGGGTTATCAGATCCACAC TGGTGGCCACCTCGCCTCGGAGGGGCTCATTGGTCCAGCAGAGTCGACCTCCTTTTCTTGAACAGAAAGGGGGGAGG GGGTCTAGCATGAGCTCATCAGGGGGGTCCGCATCTATGGTAAATATTCCCGGTAGCAAATCTTTGTCAAAATAGCT GATGGTGGTGGGATCATCCAAGGTCATCTGCCATTCTCGAACTGCCAGCGCGCGCTCATAGGGGTTAAGAGGGGTGC CCCAGGGCATGGGGTGGGTGAGCGCGGAGGCATACATGCCACAGATATCGTATACATAGAGGGGCTCTTCGAGGATG CCGATGTAAGTGGGATAACAGCGCCCCCCTCTGATGCTTGCTCGCACATAGTCATAGAGTTCATGTGAGGGGGCGAG AAGACCCGGGCCCAGATTGGTGCGGTTGGGTTTTTCCGCCCTGTAAACGATCTGGCGAAAGATGGCATGGGAATTTG AAGAGATAGTAGGTCTCTGGAATATGTTAAAATGGGCATGAGGTAGGCCTACAGAGTCCCTTATGAAGTGGGCATAT GACTCTTGCAGCTTGGCTACCAGCTCGGCGGTGACGAGTACATCCAGGGCACAGTAGTCGAGAGTTTCCTGGATGAT GTCATAACGCGGTTGGCTTTTCTTTTCCCACAGCTCGCGGTTGAGAAGGTATTCTTCGCGATCCTTCCAGTACTCTT CGAGGGGAAACCCGTCTTTTTCTGCACGGTAAGAGCCCAACATGTAGAACTGATTGACTGCCTTGTAGGGACAGCAT CCCTTCTCCACTGGGAGAGAGTATGCTTGGGCTGCATTGCGCAGCGAGGTATGAGTGAGGGCAAAAGTGTCCCTGAC CATGACTTTGAGGAATTGATACTTGAAGTCCATGTCATCACAGGCCCCCTGTTCCCAGAGTTGGAAGTCCACCCGCT TCTTGTAGGCGGGGTTGGGCAAAGCGAAAGTAACATCATTGAAGAGGATCTTGCCGGCCCTGGGCATGAAATTTCGG GTGATTCTGAAAGGCTGAGGGACCTCTGCTCGGTTATTGATAACCTGAGCGGCCAAGACGATCTCATCAAAGCCATT GATGTTGTGCCCCACTATGTACAGTTCTAAGAATCGAGGGGTGCCCCTGACATGAGGCAGCTTCTTGAGTTCTTCAA AAGTGAGATCTGTAGGGTCAGTGAGAGCATAGTGTTCGAGGGCCCATTCGTGCACGTGAGGGTTCGCTTTGAGGAAG GAGGACCAGAGGTCCACTGCGAGTGCTGTTTGTAACTGGTCCCGGTATTGACGAAAATGCTGCCCGACTGCCATTTT TTCTGGGGTGACGCAATAGAAGGTTTGGGGGTCCTGCCGCCAGCGATCCCACTTAAGTTTCATGGCGAGGTCATAGG CGATGTTGACGAGCCGCTGGTCTCCAGAGAGTTTCATGACCAGCATGAAGGGGATTAGCTGCTTGCCAAAGGACCCC ATCCAGGTGTAGGTTTCCACATCGTAGGTGAGGAAGAGCCTTTCTGTGCGAGGATGAGAGCCAATCGGGAAGAACTG GATCTCCTGCCACCAGTTGGAGGAATGGCTGTTGATGTGATGGAAGTAGAACTCCCTGCGACGCGCCGAGCATTCAT GCTTGTGCTTGTACAGACGGCCGCAGTACTCGCAGCGATTCACGGGATGCACCTCATGAATGAGTTGTACCTGACTT CCTTTGACGAGAAATTTCAGTGGAAAATTGAGGCCTGGCGTTTGTACCTGGCGCTCTACTATGTTGTCTGCATCGGC ATGACCATCTTCTGTCTCGATGGTGGTCATGCTGACGAGCCCTCGCGGGAGGCAAGTCCAGACCTCGGCGCGGCAGG GGCGGAGCTCGAGGACGAGAGCGCGCAGGCCGGAGCTGTCCAGGGTCCTGAGACGCTGCGGAGTCAGGTTAGTAGGC AGTGTCAGGAGATTGACTTGCATGATCTTTTCGAGGGCGTGAGGGAGGTTCAGATGGTACTTGATCTCCACGGGTCC GTTGGTGGAGATGTCAATGGCTTGCAGGGTTCCGTGCCCCTTGGGCGCTACCACCGTGCCCTTGTTTTTCCTTTTGG GCGGCGGTGGCTCTGTTGCTTCTTGCATGTTTAGGAGCGGTGTCGAGGGCGCGCACCGGGCGGCAGGGGCGGCTCGG GACCCGGCGGCATGGCTGGCAGTGGTACGTCGGCGCCGCGCGCGGGTAGGTTCTGGTACTGCGCCCTGAGAAGACTC GCATGTGCGACGACGCGGCGGTTGACATCCTGGATCTGACGCCTCTGGGTGAAAGCTACCGGCCCCGTGAGCTTGAA CCTGAAAGAGAGTTCAACAGAATCAATCTCGGTATCGTTGACGGCGGCTTGCCTAAGGATTTCTTGCACGTCGCCAG AGTTATCCTGGTAGGCGATCTCGGCCATGAACTGCTGGATCTCTTCCTCTTGAAGATCTCCGCGGCCCGCTCTCTCG ACGGTGGCCGCTAGGTCGTTGGAGATGCGCCCAATGAGTTGAGAGAATGCATTCATGCCCGCCTCGTTCCAGACGCG GCTGTAGACCACAGCCCCCACGGGATCTCTCGCGCGCATAACCACCTGGGCGAGGTTAAGCTCTACGTGGCGGGTGA AGACCGCATAGTTGCATAGGCGCTGGAAAAGGTAGTTGAGTGTGGTGGCGATGTGCTCGGTGACGAAGAAATACATG ATCCATCGTCTCAGCGGCATCTCGCTGACATCGCCCAGCGCTTCCAAGCGCTCCATGGCCTCGTAGAAGTCCACGGC AAAGTTGAAAAACTGGGAGTTACGCGCGGACACGGTCAACTCCTCTTCCAGAAGACGGATGAGTTCGGCAATGGTGG TGCGCACCTCGCGCTCGAAATCCCCCGGGATTTCTTCCTCAATCTCTTCTTCTTCCACTAACATCTCTTCCTCTTCA GGTGGGGCTGCAGGAGGAGGGGGAACGCGGCGACGCCGGCGGCGCACGGGCAGACGGTCGATGAATCTTTCAATGAC CTCTCCGCGGCGGCGGCGCATGGTCTCGGTGACGGCACGACCGTTCTCCCTGGGTCTCAGAGTGAAGACGCCTCCGC GCATCTCCCTGAAGTGGTGACTGGGAGGCTCTCCGTTGGGCAGGGACACCGCGCTGATTATGCATTTTATCAATTGC CCCGTAGGTACTCCGCGCAAGGACCTGATCGTCTCAAGATCCACGGGATCTGAAAACCTTTCGACGAAAGCGTCTAA CCAGTCGCAATCGCAAGGTAGGCTGAGCACTGTTTCTTGCGGGCGGGGGGGGCTAGACGCTCGGTCGGGGTTCTCTC TTTCTTTTCCTTCCTCCTCTTGGGAGGATGAGACGATGCTGCTGGTGATGAAATTAAAATAGGCAGTTTTGAGACGG CGGATGGTGGCGAGGAGCACCAGGTCTTTGGGTCCGGCTTGTTGGATGCGCAGGCGATGGGCCATTCCCCAAGCATT ATCCTGACATCTGGCCAGATCTTTATAGTAGTCTTGCATGAGTCGTTCCACGGGCACTTCTTCTTCGCCCGCTCTGC CATGCATGCGAGTGATCCCGAACCCGCGCATGGGCTGGACAAGTGCCAGGTCCGCTACAACCCTTTCGGCGAGGATG GCTTGCTGCACCTGGGTGAGGGTGGCTTGGAAGTCGTCAAAGTCCACAAAGCGGTGGTAGGCCCCGGTGTTGATTGT GTAGGAGCAGTTGGCCATGACTGACCAGTTGACTGTCTGGTGCCCAGGGCGCACGAGCTCGGTGTACTTGAGGCGCG AGTATGCGCGGGTGTCAAAGATGTAATCGTTGCAGGTGCGCACCAGGTACTGGTAGCCGATGAGAAAGTGTGGCGGT GGCTGGCGGTACAGGGGCCATCGCTCTGTAGCCGGGGCTCCGGGGGCAAGGTCTTCCAGCATGAGGCGGTGGTAACC GTAGATGTACCTGGACATCCAGGTGATACCGGAGGCGGTGGTGGATGCCCGCGGGAACTCGCGTACGCGGTTCCAGA TGTTGCGCAGCGGCATGAAGTAGTTCATGGTAGGCACGGTTTGGCCCGTGAGACGTGCACAGTCGTTGATGCTCTAG ACATACGGGCAAAAACGAAAGCGGTCAGCGGCTCGTCTCCGTGGCCTGGAGGCTAAGCGAACGGGTTGGGCTGCGCG TGTACCCCGGTTCGAATCTCGGATCAGGCTGGAGCCGCAGCTAACGTGGTACTGGCACTCCCGTCTCGACCCAGGCC TGCACAAAACCTCCAGGATACGGAGGCGGGTCGTTTTTTTGCTTTTTCCTGGATGGGAGCCAGTGCTGCGTCAAGCT TTAGAACACTCAGTTCTCGGGGCTGGGAGTGGCTCGCGCCCGTAGTCTGGAGAATTAATCGCCAGGGTTGCGTTGCG GTGTGCCCCGGTTCGAGTCTTAGCGCGCCGGATCGGCCGGTTTCCGCGACGTTTCTAAGACCCCGCCAGCCGACTTC TCCAGTTTACGGGAGCGAGCCCTCTTTTTTTTTTTTGTTTTTTGTTGCCCAGATGCATCCCGTGCTGCGACAGATGC GCCCCCAGCAACAGCCCCCTTCTCAGCAGCAGCTACAACAACAGCCACAAAAGGCTCTTCCTGCTCCTGTAACTACT GCGGCTGCAGCCGTCAGCGGCGCGGGACAGCCCGCCTATGATCTGGAATTGGAAGAGGGCGAGGGACTGGCGCGCCT GGGCGCACCATCGCCCGAGCGGCACCCGCCCAGCCGACTTCTCCAGTTTACGGGAGCGAGCCCTCTTTTTTTTTTTT GTTTTTTGTTGCCCAGATGCATCCCGTGCTGCGACAGATGCGCCCCCAGCAACAGCCCCCTTCTCAGCAGCAGCTAC AACAACAGCCACAAAAGGCTCTTCCTGCTCCTGTAACTACTGCGGCTGCAGCCGTCAGCGGCGCGGGACAGCCCGCC TATGATCTGGAATTGGAAGAGGGCGAGGGACTGGCGCGCCTGGGCGCACCATCGCCCGAGCGGCACCCGCGGGTGCA ACTGAAAAAGGACTCTCGCGAGGCGTACGTGCCCCAGCAGAACCTGTTCAGGGACAGGAGCGGTGAGGAGCCAGAGG AGATGCGAGCATCTCGATTTAACGCGGGTCGCGAGCTGCGCCACGGTCTGGATCGAAGACGGGTGCTGCAAGACGAG GATTTTGAGGTCGATGAAGTGACAGGGATCAGCCCAGCTAGGGCACATGTGGCCGCGGCCAACCTAGTCTCAGCCTA CGAGCAGACCGTGAAGGAGGAGCGCAACTTCCAAAAATCTTTTAACAACCATGTGCGCACCCTGATCGCCCGCGAGG AAGTGACCCTGGGTCTGATGCATCTGTGGGACCTGATGGAGGCTATCACCCAGAACCCCACTAGCAAACCACTGACA GCTCAGCTGTTTCTGGTGGTTCAACATAGCAGGGACAACGAGGCATTCAGGGAGGCGTTGTTGAACATCACCGAGCC TGATGGGAGATGGCTGTATGATCTGATCAACATCCTGCAAAGTATTATAGTGCAGGAACGTAGCCTGGGTTTGGCTG AGAAAGTGGCAGCTATCAACTACTCGGTCTTGAGCCTGGGCAAATACTACGCTCGCAAGATCTACAAGACCCCCTAC GTACCCATAGATAAGGAGGTAAAGATAGATGGGTTTTACATGCGCATGACTCTGAAGGTGCTGACTCTGAGCGACGA TCTGGGGGTGTATCGCAATGACAGGATGCACCGCGCGGTGAGCGCCAGCAGGAGGCGCGAGCTGAGCGACAGAGAAC TTATGCACAGCTTGCAAAGAGCTCTAACGGGGGCCGGGACTGATGGGGAGAACTACTTTGACATGGGAGCGGACTTG CAATGGCAACCCAGTCGCAGGGCCATGGAGGCTGCAGGGTGTGAGCTTCCTTACATAGAAGAGGTGGATGAAGTCGA GGACGAGGAGGGCGAGTACTTGGAAGACTGATGGCGCGACCCGTATTTTTGCTAGATGGAACAGCAGCAGGCACCGG ACCCCGCAATGCGGGCGGCGCTGCAGAGCCAGCCGTCCGGCATTAACTCTTCGGACGATTGGACCCAGGCCATGCAA CGCATAATGGCGCTGACGACCCGCAACCCCGAAGCCTTTAGACAGCAACCCCAGGCCAACCGCCTTTCGGCCATACT GGAGGCCGTAGTGCCCTCCCGCTCCAACCCCACCCACGAGAAGGTCCTGGCTATCGTGAACGCGCTGGTGGAGAACA AGGCCATCCGTCCCGATGAGGCCGGGCTGGTATACAATGCTCTCTTGGAGCGCGTGGCCCGTTACAACAGCAGCAAC GTGCAAACCAACCTGGACAGGATGGTGACCGATGTGCGCGAGGCCGTGTCTCAGCGCGAGCGGTTCCAGCGCGGCGC CAACTTGGGGTCGTTGGTAGCGCTAAACGCTTTCCTCAGCACCCAGCCCGCCAACGTGCCCCGTGGTCAGCAAGACT ATACAAACTTTTTGAGTGCATTGAGACTCATGGTAGCTGAGGTGCCCCAGAGCGAGGTGTACCAGTCCGGGCCAGAT TACTTCTTCCAGACCAGCAGACAGGGCTTGCAGACAGTGAACCTGACTCAGGCTTTCAAGAACCTGAAGGGTCTGTG GGGAGTGCACGCCCCAGTAGGAGATCGCGCGACCGTGTCTAGCTTGCTGACTCCCAACTCCCGCCTGCTGCTGCTGC TGGTATCCCCCTTTACTGACAGCGGTAGCATCGACCGCAACTCGTACTTGGGCTACCTGCTTAACCTGTATCGCGAG GCCATAGGGCAGAGCCAGGTGGACGAGCAGACCTATCAAGAAATCACCCAAGTGAGCCGCGCCCTGGGTCAGGAAGA CACGGGCAGTTTGGAAGCCACCCTGAACTTCTTGCTAACCAACCGGTCGCAGAAGATCCCTCCTCAGTATGCGCTTA CCGCTGAGGAGGAGCGGATCCTCAGATACGTGCAACAGAGCGTTGGACTGTTTCTGATGCAGGAGGGGGCGACACCT ACCGCCGCGCTGGACATGACAGCTCGAAACATGGAGCCCAGCATGTATGCTAGTAACAGGCCTTTCATTAACAAACT GCTGGACTACCTGCACAGGGCGGCCGCCATGAACTCTGATTATTTCACCAATGCTATCCTGAACCCACACTGGCTGC CCCCACCTGGTTTCTACACTGGCGAGTACGACATGCCCGACCCCAATGACGGGTTCCTGTGGGACGATGTGGACAGC AGCATATTCTCCCCGCCTCCCGGTTATACAGTTTGGAAGAAGGAAGGGGGCGATAGAAGACACTCTTCCGTGTCGCT GTCCGGAACGGCTGGTGCTGCCGCGACCGTGCCCGAAGCTGCAAGTCCTTTCCCTAGCTTGCCCTTTTCACTAAACA GCGTTCGCAGCAGTGAACTGGGGAGAATAACCCGCCCGCGCTTGATGGGCGAGGATGAGTACTTGAATGACTCTTTG CTGAGGCCAGAGAGGGAAAAGAACTTCCCCAACAATGGAATAGAGAGTCTGGTGGATAAGATGAGTAGATGGAAGAC CTATGCGCAGGATCACAGAGACGAGCCCAGGATATTGGGGGCTACAAGCAGACCGACCCGTAGACGCCAGCGCCACG ACAGACAGATGGGTCTTGTGTGGGACGATGAGGACTCTGCCGATGATAGCAGCGTGTTGGACTTGGGTGGAAGAGGA GGGGGCAACCCGTTCGCTCATCTGCGTCCCAGATTCGGGCGCATGTTGTAAAAGTGAAAGTAAAATAAAAATGCAAC TCACCAAGGCCATGGCGACCGAGCGTGCGTTCGTTCTTTTTTGTTATCTGTGTCTAGTACGATGAGGAGACGAGCCG TGCTAGGCGGAGCGGTGGTGTATCCGGAGGGTCCTCCTCCTTCTTACGAGAGCGTGATGCAGCAACAGGCGGCGATG ATACAGCCCCCACTGGAGGTTCCCTTCGTACCCCCGCGGTACCTGGCGCCTACGGAAGGGAGAAACAGCATTCGTTA CTCGGAGCTGTCGCCCCTGTACGATACCACCAAGTTGTATCTGGTTGACAACAAGTCGGCGGACATCGCCTCCCTGA ACTATCAGAACGACCACAGCAACTTCCTGACCACGGTGGTGCAGAACAATGACTTTACCCCCACGGAGGCTAGCACC CAGACCATCAACTTTGACGAACGGTCGCGATGGGGCGGTCATCTGAAGACCATCATGCACACCAACATGCCCAACGT GAACGAGTACATGTTCAGCAACAAGTTCAAGGCGAGGGTGATGGTGTCCAGAAAAGCTCCTGAAGGTGTTACAGTAA ATGACACCTATGATCATAAAGAGGATATCTTGAAGTATGAGTGGTTTGAGTTCATTTTACCAGAAGGCAACTTTTCA GCCACCATGACGATCGACCTGATGAACAATGCCATCATTGACAACTACCTGGAAATTGGCAGACAGAATGGAGTGCT GGAAAGTGACATTGGTGTTAAGTTTGACACTAGAAATTTCAGGCTCGGGTGGGACCCCGAAACTAAGTTGATTATGC CAGGAGTCTACACTTATGAGGCATTCCATCCTGACATTGTATTGCTGCCTGGTTGCGGGGTAGACTTTACTGAAAGC CGACTTAGCAACTTGCTTGGCATCAGGAAGAGACATCCATTCCAGGAGGGTTTCAAAATCATGTATGAAGATCTTGA AGGGGGTAATATTCCTGCCCTTTTGGATGTCACTGCCTATGAGGAAAGCAAAAAGGATACCACTACTGAAACAACCA CACTGGCTGTTGCAGAGGAAACTAGTGAAGATGATAATATAACTAGAGGAGATACCTATATAACAGAAAAACACAAA CGTGAAGCTGCAGCTGCTGAAGTTAAAAAAGAGTTAAAGATCCAACCTCTAGAAAAAGACAGCAAGAGTAGAAGCTA CAATGTCTTGGAAGACAAAATCAACACGGCCTACCGCAGTTGGTACCTGTCCTACAATTACGGTAACCCTAAGAAAG GAATAAGGTCTTGGACACTGCTCACCACTTCAGATGTCACCTGTGGGGCAGAGCAGGTTTACTGGTCGCTCCCTGAC ATGATGCAAGACCCAGTCACGTTCCGCTCCACAAGACAAGTCAACAACTACCCAGTGGTGGGTGCAGAGCTTATGCC CGTCTTCTCAAAGAGTTTCTACAATGAGCAAGCCGTGTACTCTCAGCAGCTCCGACAGGCCACTTCGCTCACGCACG TCTTCAACCGCTTCCCTGAGAACCAGATCCTCATCCGCCCGCCGGCGCCCACAATTACCACCGTCAGTGAAAACGTT CCTGCTCTCACAGATCACGGGACCCTGCCGTTACGCAGCAGTATCCGGGGAGTCCAGCGCGTGACCGTTACTGACGC CAGACGCCGCACCTGTCCCTACGTTTACAAGGCCCTGGGCATAGTCGCGCCGCGCGTTCTTTCAAGCCGCACTTTCT AAAAAAAAAAAATGTCCATTCTCATCTCGCCCAGTAATAATACCGGTTGGGGACTGTATGCGCCCACCAAGATGTAT GGAGGCGCCCGCAAACGCTCTACCCAGCACCCTGTGCGCGTTCGCGGTCATTTCCGCGCTCCCTGGGGCGCACTCAA GGGTCGTACCCGCACTCGGACCACGGTCGATGATGTGATCGACCAGGTGGTCGCCGATGCTCGTAATTATACTCCTA CTGCGCCTACATCTACTGTGGATGCAGTTATTGACAGTGTGGTGGCAGACGCCCGCGCCTATGCTCGCCGGAAGAGC CGAAGGAGGCGCATCGCCAGGCGCCACAGGGCTACTCCCGCCATGCGAGCTGCAAAAGCTATTCTGCGGAGGGCCAA ACGTGTGGGGCGAAGAGCCATGCTTAGAGCGGCCAGACGCGCGGCTTCAGGTGCCAGCAGCGGCAGGTCCCGCAGGC GCGCGGCCACGGCGGCAGCAGCGGCCATTGCCAACATGGCCCAACCGCGAAGAGGCAATGTGTACTGGGTGCGTGAT GCCACTACCGGCCAGCGCGTGCCCGTGCGCACTCGCCCCCCTCGCACTTAGAAGATACTGAGCAGTCTCCGATGTTG TGTCCCAGCGGCAAGTATGTCCAAGCGCAAATACAAGGAAGAGATGCTCCAGGTCATCGCGCCTGAAATCTACGGTC CACCGGTGAAGGATGAAAAAAAGCCCCGCAAAATCAAGCGGGTCAAAAATAACAAAAAGGAAGAAGATGACGATGAT GGGCTGGTGGAGTTTGTGCGCGAGTTCGCCCCAAGACGGCGCGTGCAGTGGCGCGGGCGCAAAGTGCGTCAAGTGCT CAGACCCGGGACCACTGTGGTTTTTACACCCGGCGAGCGTTCCAGCACTACTTTTAAACGGTCCTATGATGAGGTGT ACGGGGATGACAATATTCTTGAGCAGGCGGCAGACCGCCTTGACGAGTTTGCTTATGGCAAGCGCACTAGATCCAGT CCCAAAGAGGAGGCGGTGTCCATTCCTTTGGATCATGGAAATCCCACCCCCAGCCTCAAACCAGTCACCCTGCAGCA AGTGCTGCCCGTGCCTGCGCGGAGAGGCGTAAAGCGCGAGGGTGAGGACCTGTATCCCACCATGCAGCTAATGGTGC CCAAGCGCCAGAGGCTAGAAGACGTACTGGAGAAAATGAAAGTGGATGCCGATATCCAGCCTGAGGTCAAAGTAAGA CCTATCAAGGAAGTGGCGCCAGGTTTGGGAGTACAAACCTTCGACATCAAGATTCCCACCGAGTCCATGGAAGTGCA GACCGAACCTGCAAAACCCACAACCACCTCAATTGAGGTGCAAACGGAACCCTGGATGCCCGCGCCCGTTGCCGCCC CCAGCACCACTCGAAGATCACGACGAAAGTACGGCCCAGCAAGTCTGCTAATGCCCAACTATGCTCTGCACCCATCC ATCATTCCCACTCCGGGTTACAGAGGCACTCGCTACTATCGAAACCGGAGCAACACCTCTCGCCGCCGCAAACCACC TGCAAGTCGCACTCGCCGTCGCCGCCGCCGCAACACTGCCAGCAAATTGACTCCCGCCGCCCTGGTGCGGAGAGTGT ACCGCGATGGTCGCGCTGAACCTCTGACGCTGCCGCGCGCGCGCTACCATCCAAGCATCACCACTTAATGACTGTTG ACGCTGCCTCCTTGCAGATATGGCCCTCACTTGCCGCCTTCGCGTCCCCATTACTGGCTACCGAGGAAGAAACTCGC GCCGTAGAAGGATGTTGGGGCGAGGGATGCGCCGCCACAGACGAAGGCGCGCTATCAGCAGACGATTAGGGGGTGGC TTTTTGCCAGCTCTTATACCCATCATCGCCGCAGCGATCGGGGCGATACCAGGCATAGCTTCCGTGGCGGTTCAGGC CTCGCAGCGCCACTAACATTGGAAAAACTTATAAATAAAAAATAGAATGGACTCTGACGCTCCTGGTCCTGTGACTA TGTTTTTGTAGAGATGGAAGACATCAATTTTTCATCCCTGGCTCCGCGACACGGCACGAGGCCGTACATGGGCACCT GGAGCGACATCGGCACGAGCCAACTGAACGGGGGCGCCTTCAATTGGAGCAGTATCTGGAGCGGGCTTAAAAATTTT GGCTCGACCGTAAAAACCTATGGGAACAAAGCTTGGAACAGCAGCACAGGGCAGGCTCTGAGAAATAAGCTTAAGGA ACAAAACTTCCAACAGAAGGTGGTCGATGGGATCGCCTCTGGTATTAACGGCGTAGTGGATCTGGCCAACCAGGCTG TACAAAAACAGATAAACAGCCGCCTGGACCCGCCGCCCGCAACCCCTGGTGAAATGGAAGTGGAGGAAGAACTTCCT CCGCTGGAAAAGCGGGGCGACAAGCGTCCGCGACCCGAGCTAGAGCACACGCTGGTGACGCGCGCAGACGAGCCCCC TTCATACGAGGAGGCAGTAAAGCTCGGAATGCCCACTACCAGGCCCGTAGCTCACATGGCTACCGGGGTGATGAAAC CTTCTGAGTTACATCGACCCGCCACCTTGGACTTGCCTCCTCCCCCTGCTTCTGCGGCGCCTGTTCCCAAACCTGTC GCTACCAGAAAGCCCACCGCCGTACAGCCCGTTGCCGTAGCCAGACCGCGTCCTGGGGGCACACCGCGCCCGAAAGC AAACTGGCAAAGTACTCTGAACAGCATCGTGGGTCTGGGCGTGCAGAGTGTAAAGCGCCGTCGCTGCTATTAATTAA ATATGGAGTAGCGCTTAACTTGCTTGTCTGTGTGTATGTATCATCACCACGCCGCCGCAGCAGAGGAGAAAGGAAGA GGTCGCGCGCCGAGGCTGAGTTGCTTTCAAGATGGCCACCCCATCGATGATGCCCCAATGGGCATACATGCACATCG CCGGACAGGATGCTTCGGAGTACCTGAGTCCGGGTCTGGTGCAGTTCGCCCGTGCAACAGACACCTACTTCAGTATG GGGAACAAGTTTAGAAACCCCACAGTGGCGCCCACCCACGATGTGACCACCGACCGTAGCCAGCGACTGATGCTGCG CTTCGTGCCCGTTGACCGGGAGGACAATACATACTCTTACAAAGTGCGGTACACCCTCGCCGTGGGCGACAACAGAG TGCTTGACATGGCCAGCACATTCTTTGACATTAGGGGGGTGCTTGATAGAGGTCCTAGCTTCAAGCCATATTCCGGC ACAGCTTACAATTCACTGGCTCCTAAGGGCGCGCCTAACACATCTCAGTGGATAGTTACAACGGGAGAAGACAATGC CACCACATACACATTTGGCATTGCTTCCACGAAGGGAGACAATATTACTAAGGAAGGTTTAGAAATTGGGAAAGACA TTACTGCAGACAACAAGCCCATTTATGCCGATAAAACATATCAGCCAGAGCCTCAAGTTGGAGAAGAATCATGGACT GATATTGATGGAACAAATGAAAAATTTGGAGGTAGAGCTCTTAAACCAGCTACTAAAATGAAGCCATGCTACGGGTC TTTTGCAAGACCTACAAACATAAAAGGGGGCCAAGCTAAAAACAGAAAAGTAACACCAACCGAAGGAGATGTTGAAG CTGAGGAGCCAGATATTGATATGGAATTTTTCGATGGTAGAGAAGCTGCTGACGCTTTTTCGCCTGAAATTGTGCTT TACACGGAAAATGTCAATTTGGAAACTCCAGACAGCCATGTGGTATACAAGCCAGGAACTTCTGATGGTAACTCTCA TGCAAATTTGGGTCAACAAGCCATGCCTAACAGACCCAATTACATTGGCTTCAGGGATAACTTTGTAGGTCTTATGT ACTACAACAGTACTGGAAATATGGGAGTTTTGGCCGGCCAAGCATCACAACTGAATGCAGTGGTTGACTTGCAGGAC AGAAACACTGAACTGTCATATCAGCTTTTGCTTGATTCTCTGGGAGACAGAAGCAGATACTTCAGCATGTGGAATCA GGCTGTGGACAGCTATGATCCCGATGTTCGTATTATTGAAAATCATGGCGTCGAGGATGAACTGCCTAATTACTGTT TTCCTCTGGATGGCATAGGACCAGGGAACAAATATCAAGGCATTAAACCTAGAGACACTGCATGGGAAAAAGATACT AAAGTTTCTACAGCTAATGAAATAGCCATAGGCAACAATCTGGCTATGGAAATTAATATCCAAGCTAATCTTTGGAG AAGTTTTCTGTACTCCAATGTGGCTTTGTACCTTCCAGATGTTTACAAGTACACGCCAACTAACATTACTCTGCCCG CTAACACCAACACCTATGAGTACATGAACGGGCGAGTGGTTTCCCCATCTCTGGTCGATTCATACATCAACATTGGC GCCAGGTGGTCTCTTGACCCAATGGACAATGTGAATCCATTTAACCACCACCGCAATGCTGGCCTACGCTACCGGTC CATGCTTCTGGGCAATGGCCGTTATGTGCCTTTCCACATACAAGTGCCTCAAAAATTCTTTGCTGTCAAGAACCTAC TTCTTCTACCTGGCTCCTACACCTATGAGTGGAACTTCAGAAAGGATGTGAACATGGTCCTGCAAAGTTCCCTTGGA AATGACCTCAGAACAGATGGTGCTAACATAAGTTTCACCAGCATCAACCTCTATGCCACCTTCTTCCCCATGGCTCA CAACACCGCTTCAACTCTTGAAGCCATGCTGCGCAACGATACCAATGATCAGTCATTCAACGACTACCTCTCTGCAG CTAACATGCTTTACCCCATCCCTGCCAATGCAACCAACATTCCAATTTCCATCCCATCTCGCAACTGGGCAGCCTTC AGGGGCTGGTCCTTCACCAGACTCAAAACCAAGGAGACTCCATCTCTTGGATCAGGGTTCGATCCCTACTTCGTTTA TTCTGGATCTATTCCCTACCTGGATGGCACTTTTTACCTTAACCACACTTTCAAGAAGGTCTCCATCATGTTTGACT CCTCAGTCAGCTGGCCTGGCAATGACAGGCTGTTGTCTCCAAATGAGTTTGAAATCAAGCGCACTGTGGATGGGGAA GGATACAATGTGGCCCAATGCAACATGACCAAAGACTGGTTCCTGGTTCAGATGCTTGCCAACTACAACATTGGCTA CCAGGGCTTTTACATCCCTGAGGGATACAAGGATCGCATGTACTCCTTTTTCAGAAACTTCCAGCCTATGAGCAGGC AGGTGGTTGATGAGGTTAATTACACTGACTACAAAGCCGTCACCTTACCATATCAACACAACAACTCTGGCTTTGTA GGATACCTTGCGCCTACTATGAGACAAGGGGAACCTTACCCAGCCAATTATCCATACCCGCTCATCGGAACTACTGC CGTTAAAAGTGTTACCCAAAAAAAGTTCCTGTGCGACAGGACCATGTGGCGCATACCGTTCTCCAGCAACTTCATGT CCATGGGAGCCCTTACGGACCTGGGACAGAACCTGCTCTATGCCAACTCGGCCCATGCGCTGGACATGACTTTTGAG GTGGATCCCATGGATGAGCCCACCCTGCTTTATCTTCTTTTCGAAGTCTTCGACGTGGTCAGAGTGCACCAGCCACA CCGCGGCGTCATCGAGGCCGTCTACCTGCGCACACCGTTCTCGGCCGGCAACGCCACCACATAAGAAGCCTCTTGCT TCTTGCAAGCAGCAGCTGCAGCCATGTCATGCGGGTCCGGAAACGGCTCCAGCGAGCAAGAGCTCAAAGCCATCGTC CGAGACCTGGGTTGCGGACCCTATTTCCTGGGAACCTTTGACAAGCGTTTCCCGGGGTTCATGGCCCCCGACAAGCT CGCCTGCGCCATAGTCAACACTGCCGGACGCGAGACGGGGGGAGAGCACTGGCTGGCTTTTGGTTGGAACCCGCGCT CCAACACCTGCTACCTTTTTGATCCTTTTGGGTTCTCGGATGAGCGACTCAAACAGATTTACCAGTTTGAGTACGAG GGGCTCCTGCGCCGCAGTGCCCTTGCTACCAAAGACCGCTGCATCACCCTGGAAAAGTCCACCCAGAGCGTGCAGGG CCCACGCTCAGCCGCCTGTGGACTTTTTTGCTGTATGTTCCTTCATGCCTTTGTGCACTGGCCCGACCGCCCCATGA ACGGAAACCCCACCATGAAGTTGCTGACTGGGGTGCCCAACAGCATGCTCCAATCTCCCCAAGTGCAGCCCACCCTG CGCCGCAACCAGGAGGCGCTATATCGCTTCCTAAACACCCACTCATCTTACTTTCGTTCTCACCGCGCACGCATCGA AAGGGCCACCGCGTTTGACCGTATGGATATGCAATAAGTCATGTAAAACCGTGTTCAATAAAAAGCACTTTATTTTT ACATGCACTAAGGCTCTCGTTTTTTACTCATTCGTTTTCATTATTCACTCAGAAATCAAATGGGTTCTGGCGGGAGT CAAAGTGACCCGCGGGCAGGGATACGTTGCGGAACTGTAACCTGTTCTGCCACTTGAACTCGGGGATCACCAACTTG GGAACTGGAATCTCGGGAAAGGTGTCTTGCCACAACTTTCTGGTCAGCTGCAGGGCGCCAAGTAGGTCAGGAGCAGA GATCTTGAAATCACAGTTGGGACCGGCATTCTGGACACGGGAGTTGCGGTACACTGGGTTGCAACACTGGAACACCA TCAAGGCTGGGTGTCTCACGCTTGCCAGCACGGTCGGGTCACTGATGGTAGTCACATCCAAGTCTTCAGCATTGGCC ATCCCAAAGGGGGTCATCTTACAGGTCTGCCTGCCCATCACGGGAGCGCAGCCTGGCTTGTGGTTGCAATCGCAATG AATGGGGATCAGCATCATCCTGGCTTGGTCGGGGGTTATCCCTGGGTACACGGCCTTCATGAAGGCTTCGTACTGCT TGAAAGCTTCCTGAGCCTTACTTCCCTCGGTATAGAACATCCCACAGGACTTGCTGGAAAATTGATTAGTAGCACAG TTGGCATCATTTACACAGCAGCGGGCATCGTTGTTGGCCAACTGGACCACATTTCTGCCCCAGCGGTTCTGGGTGAT CTTGGCTCTGTCTGGGTTCTCCTTCATAGCGCGCTGTCCGTTCTCGCTCGCCACATCCATCTCGATAATGTGGTCCT TCTGAATCATGATAGTGCCATGCAGGCATTTCACCTTGCCTTCATAATCGGTGCATCCATGAGCCCACAGAGCGCAC CCGGTGCACTCCCAACTATTGTGGGCGATCTCAGAATAAGAATGTACCAATCCCTGCATGAATCTTCCCATCATCGC TGTCAGGGTCTTCATGCTACTAAATGTCAGCGGGATGCCACGGTGCTCCTCGTTCACATACTGGTGGCAGATACGCT TGTACTGCTCGTGCTGCTCTGGCATCAGCTTGAAAGAGGTTCTCAGGTCATTATCCAGCCTGTACCTCTCCATTAGC ACAGCCATCACTTCCATGCCCTTCTCCCAGGCAGATACCAGGGGCAAGCTCAAAGGATTCCTAACAGCAATAGAAGT AGCTCCTTTAGCTATAGGGTCATTCTTGTCGATCTTCTCAACACTTCTCTTGCCATCCTTCTCAATGATGCGCACCG GGGGGTAGCTGAAGCCCACGGCCACCAACTGAGCCTGTTCTCTTTCTTCTTCGCTGTCGTGGCCGATGTCTTGCAGA GGGACATGCTTGGTCTTTCTGGGCTTCTTCTTGGGAGGGATCGGGGGAGGACTGTTGCTCCGTTCCGGAGACAGGGA TGACCGCGAAGTTTCGCTTACCAGTACCACCTGGCTCTCGATAGAAGAATCGGACCCCACGCGACGGTAGGTGTTCC TCTTCGGGGGCAGAGGTGGAGGCGACTGAGATGGGCTGCGGTCTGGCCTTGGAAGCGGATGGCTGGCAGAGCCCATT CCGCGTTCGGGGGTGTGCTCCCGTTGGCGGTCGCTTGACTGATTTCCTCCGCGGCTGGCCATTGTGTTCTCCTAGGC AGAGAAACAACAGACATGGAAACTCAGCCATCACTGCCAACATCGCTGCAAGCGCCATCACACCTCGCCCCCAGCAG CGACGAGGAGGAGAGCTTAACCACCCCACCACCCAGTCCAGCTACCACCACCTCTACCCTCGATGATGAGGAGGAGG AGGTCGACGCAGCCCAGGAGATGCAGGCGCAGGATAATGTGAAAGCGGAAGAGATTGAGGCAGATGTCGAGCAGGAC CCGGGCTATGTGACACCGGCGGAGCACGAGGAGGAGCTGAAACGTTTTATAGACAGAGAGGATGACGACCGCCCAGA GCATCAAGCAGATGGCGATCACCAGGAGGCTGGCATCGGGGATCAAGTTGCCGACTACCTCACCGGGCTTGGGGGGG AAGACGTGCTCCTCAAACATCTAGCAAGGCAGTCGAACATAGTTAAAGACGCACTACTCGACCTCACCGAAGTGCCC ATCAGTGTGGAAGAGCTTAGCCGCGCCTACGAGCTGAACCTCTTTTCGCCTCACATACCCCCCAAGCGGCAGCCAAA CGGCACCTGCGAGGCCAACCCTCGACTGAACTTCTATCCAGCTTTTACTGTCCCCGAAGTGCTGGCCACCTACCACA TCTTTTTTAAGAACCAAAAGATTCCAGTCTCCTGCCGCGCCAACCGCACCCGCGCCGATGCCCTTCTCAACTTGGGT CCGGGAGCTCGCTTACCTGATATAGCTTCCTTGGAAGAGGTTCCAAAAATCTTTGAGGGTCTGGGAAGTGATGAGAC TCGGGCCGCAAATGCTCTGCAACAGGGAGAGAATGGCATGGATGAACATCACAGCGCTTTAGTGGAACTGGAGGGTG ACAATGCCCGGCTTGCAGTGCTCAAGCGCAGTATCGTGGTCACCCATTTTGCCTACCCCGCTGTTAACCTGCCCCCC AAAGTTATGAGCGCTGTTATGGACCATCTGCTCATCAAACGAGCAGGTCCACTTTCAGAAAACCAGAACATGCAGGA TCCAGACGCCTCGGACGAGGGCAAGCCGGTAGTCAGTGACGAGCAGCTATCTCGCTGGCTGGGTACCAACTCCCCCC GAGATTTGGAAGAGAGGCGCAAGCTTATGATGGCTGTAGTGCTAGTAACTGTGGAGCTGGAGTGTTTGCGCCGCTTT TTCACCGACCCCGAGACCCTGCGCAAGCTAGAGGAGAACCTGCACTACACCTTTAGACATGGCTTCGTGCGGCAGGC ATGCAAGATCTCCAACGTGGAGCTTACCAACCTGGTTTCTTACATGGGCATTTTGCATGAGAACCGGCTAGGGCAGA GCGTCCTGCACACCACCCTTAAAGGGGAGGCCCGCCGTGACTACATCCGAGACTGTGTCTACCTCTACCTCTGCCAT ACCTGGCAGACTGGCATGGGTGTGTGGCAACAGTGTTTGGAAGAGCAGAACCTAAAAGAGCTGGACAAGCTCTTGCA GAGATCCCTCAAAGCCCTGTGGACAGGTTTTGATGAGCGCACCGTCGCCTCGGACCTGGCAGACATCATCTTCCCCG AGCGTCTCAGGGTTACTCTGCGAAACGGCCTGCCAGACTTTATGAGCCAGAGCATGCTTAACAACTTTCGCTCTTTC ATCCTGGAACGCTCCGGTATCCTGCCTGCCACCTGCTGTGCGCTGCCCTCCGACTTTGTGCCTCTCACCTACCGCGA GTGCCCACCGCCGCTATGGAGCCACTGCTACCTGTTCCGCCTGGCCAACTACCTCTCCTACCACTCGGATGTTATAG AGGATGTGAGCGGAGACGGTCTGCTGGAATGCCACTGCCGCTGCAATCTTTGCACACCCCACCGCTCCCTTGCCTGC AACCCCCAGTTGCTGAGCGAGACCCAGATCATCGGCACCTTCGAGTTGCAGGGTCCCAGCAGTGAAGGCGAGGGGTC TTCTCCGGGGCAGAGTCTGAAACTGACACCGGGGCTGTGGACCTCCGCCTACCTGCGCAAGTTTCATCCCGAGGATT ACCACCCCTATGAGATCAGGTTCTATGAGGACCAGTCACATCCTCCCAAAGTCGAGCTCTCAGCCTGCGTCATCACC CAGGGAGCAATTCTGGCCCAATTGCAAGCCATCCAAAAATCCCGCCAAGAATTTCTACTGAAAAAGGGAAGCGGGGT CTACCTTGACCCCCAGACCGGTGAGGAGCTCAACACAAGGTTCCCCCAGGATGTCCCATCGCCGAGGAAGCAAGAAG CTGAAGGTGCAGCTGACGCCCCCAGAGGATATGGAGGAAGACTGGGACAGTCAGGCAGAGGAGGAGATGGAAGATTG GGACAGCCAGGCAGAGGAGGTGGACAGCCTGGAGGAAGACAGTTTGGAGGAGGAAGACGAGGAGGCAGAGGAGGTGG AAGAAGCAACCGCCGCCAAACAGTTGTCATCGGCGGCGGAGACAAGCAAGTCCCCAGACAGCAGCACGGCTACCATC TCCGCTCCGGGTCGGGGGGTCCAGCGGCGGCCCAACAGTAGATGGGACGAGACCGGGCGATTCCCAAACCCGACCAC CGCTTCCAAGACCGGTAAGAAGGAGCGACAGGGATACAAGTCCTGGCGTGGACATAAAAACGCTATCATCTCCTGCT TGCATGAATGCGGGGGCAACATATCCTTCACCCGGCGATACCTGCTCTTCCACCACGGTGTGAACTTCCCCCGCAAT ATCTTGCATTACTACCGTCACCTCCACAGCCCCTACTGCAGTCAGCAAGTCCCGGCAACCCCGACAGAAAAAGACAG CAGCGACAACGGTGACCAGAAAACCAGCAGTTAGAAAATCTACAACAAGTGCAGCAGGAGGAGGACTGAGGATCACA GCGAACGAGCCAGCGCAGACCAGAGAGCTGAGGAACCGGATCTTTCCAACCCTCTATGCCATCTTCCAGCAGAGTCG GGGGCAAGAGCAGGAACTGAAAGTAAAAAACCGATCTCTGCGCTCGCTCACCAGAAGTTGTTTGTATCACAAGAGCG AAGACCAACTTCAGCGCACTCTCGAGGACGCCGAGGCTCTCTTCAACAAGTACTGCGCGCTGACTCTTAAAGAGTAG CCCTTGCCCGCGCTTATTCGAAAACGGCGGGAATCACGTCACCCTTGGCACCTGTCCTTTGCCCTAGTCATGAGTAA AGAGATTCCCACGCCTTACATGTGGAGCTATCAGCCCCAAATGGGGTTGGCAGCAGGCGCCTCCCAGGACTACTCCA CCCGCATGAATTGGCTTAGCGCCGGGCCCTCAATGATATCACGGGTTAATGATATACGAGCTTATCGAAACCAGTTA CTCCTAGAACAGTCAGCTCTCACCACCACACCCCGCCAACACCTTAATCCCCGAAATTGGCCCGCCGCCCTGGTGTA CCAGGAAACTCCCGCTCCCACCACCGTACTACTTCCTCGAGACGCCCAGGCCGAAGTTCAGATGACTAACGCAGGTG TACAGCTGGCGGGCGGTTCCGCCCTATGTCGTCACCGACCTCAACAGAGTATAAAACGCCTGGTGATCAGAGGCCGA GGTATCCAGCTCAACGACGAGTCGGTTAGCTCTTCGCTTGGTCTGCGACCAGACGGAGTCTTCCAGATCGCCGGCTG TGGGAGATCTTCCTTCACCCCTCGTCAGGCTGTACTGACTTTGGAGAGTTCGTCCTCGCAGCCCCGCTCGGGCGGCA TCGGAACTCTCCAGTTCGTGGAGGAGTTTACTCCCTCTGTCTACTTCAACCCCTTCTCCGGCTCTCCTGGCCAGTAC CCAGACGAGTTCATACCGAACTTCGACGCAATCAGCGAGTCAGTGGATGGCTATGATTGATGTCTAATGGTGGCGCG GCTGAGCTAGCTCGACTGCGACACCTAGACCACTGCCGCCGCTTTCGCTGCTTCGCCCGGGAACTCACCGAGTTCAT CTACTTCGAACTCCCCGAGGAGCACCCTCAGGGTCCGGCCCACGGAGTGCGGATTACCATCGAAGGGGGAATAGACT CTCGCCTGCATCGAATCTTCTCCCAGCGACCCGTGCTGATTGAGCGCGACCAGGGAAATACAACCATCTCCATTTAC TGCATCTGTAACCACCCCGGATTGCATGAAAGCCTTTGCTGTCTTGTTTGTGCTGAGTTTAATAAAAACTGAGTTAA GACCCTCCTACGGACTACCGCTTCTTCAATCAGGACTTTACAACACCAACCAGATCTTCCAGAAGACCCAGACCCTT CCTCCTCTGATCCAGGACTCTAACTCTACCTTACCAGCACCATCCACTACTAACCTTCCCGAAACTAACAAGCTTGG ATCTCATCTGCAACACCGCCTTTCACGAAGCCTTCTTTCTGCCAATACTACCACTCCCAAAACCGGAGGTGAGCTCC GCGGTCTCCCTACTGACGACCCCTGGGTGGTAGCGGGTTTTGTAACGTTAGGAGTAGTTGCGGGTGGGCTTGTGCTA ATCCTTTGCTACCTATACATACCTTGCTGTGCATATTTAGTCATATTGCGCTGTTGGTTTAAAAAATGGGGGCCATA TTAGTCGTGCTTGCTTTACTTTCGCTTTTGGGTCTGGGCTCTGCTAATCTCAATCCTCTTGATCACGATCCATGTCT AGACTTCGACCCAGAAAACTGCACACTTACTTTTGCACCCGACACAAGCCGTCTCTGTGGAGTTCTTATTAAGTGCG GATGGGACTGCAGGTCCGTTGAAATTACACATAATAATAAAACATGGAACAATACCTTATCCACCACATGGGAGCCA GGAGTTCCCGAGTGGTATACTGTCTCTGTCCGAGGTCCTGACGGTTCCATCCGCATTAGTAACAACACTTTTATTTT TTCTGAAATGTGCGATCTGGCCATGTTCATGAGCAGACAGTATGACCTATGGCCTCCCAGCAAAGAGAACATTGTGG CATTTTCCATTGCTTATTGCTTGGTAACATGCATCATCACTGCTATCATTTGTGTGTGCATACACTTGCTTATAGTT ATTCGCCCTAGACAAAGCAATAAGGAAAAAGAGAAAATGCCTTAACCTTTTTACTCATACCTTTTCTTTACAGCATG GCTTTTGTTACAGCTCTAATTATTGCCAACATTGTCACTGTCGCTCACGGGCAAACAATTATCCATATTACCTTAGG ACATAATCACACCCTTGTAGGGCCCCCAATTACTTCAGAGGTTATTTGGACCAAACTTGGAAGTGTTGATTATTTTG ATATAATTTGCAACAAAACTAAACCAATATTTGTAATCTGTAACAGACAAAATCTCACGTTAATTAATGTTAGCAAA ATTTATAACGGTTACTATTATGGTTATGACAGATCCAGTAGTCAATATAAAAATTACTTAGTTCGCATAACTCAGCC CAAATTAACAGTGCCCACTATGACAATAATTAAAATGGCTAATAAAGCATTAGAAAATTTTACATCACCAACAACGC CCAATGAAAAAAACATTCCAAATTCAATGATTGCAATTATTGCGGCGGTGGCATTGGGAATGGCACTAATAATAATA TGCATGTTCCTATATGCTTGTTGCTATAAAAAGTTTCAACATAAACAGGATCCACTACTAAATTTTAACATTTAATT TTTTATACAGATGTTTTCCACTACAATTTTTATCATTACTAGCCTTGCAGCTGTAACTTATGGCCGTTCACACCTAA CTCTACCTGTTGGCTCAACATGTACACTACAAGGACCCCAACAAGGCTATGTCACTTGGTGGAGAATATATGATAAT GGAGGGTTCGCTAGACCATGTGATCAGCCTGGTACAAAATTTTCATGCAACGGAAGAGACTTGACCATAATTAACAT AACATCAAATGAGCAAGGCTTCTATTATGGAACCAACTATAAAGATAGTTTAGATTACAACATTATTGTAGTGCCAG CCACCACTTCTGCTCCCCGCAAAACCACTTTCTCTAGCAGCAGTGCCAAAGCAAGCACAATTCCTAAAACAGCTTCT GCTATGTTAAAGCTTCAAAAAATCGCTTTAAATAATTCCACAGCCGCTCCCAATACAATTCCTAAATCAACAATTGG CATCATTACTGCCGTGGTAGTGGGATTAATTATTATATTTTTGTGCATAATGTACTATGCCTGCTGCTATAGAAAAC ATGAACAAAAAGGTGATGCATTACTAAATTTTGACATTTAATTTTTTATAGAATTATGATATTGTTTCAATCAAATA CCACTAACACTATCAATGTGCAGACTACTTTAAATCATGACATGGAAAACCACACTACCTCCTATGCATACACAAAC ATTCAGCCTAAATACGCTATGCAATAGAAATTCTAAAAGACGTCCCATCTATTCTCCTATGATTAGTCGTCCCCATA TGGCTTTGAATGAAATCTAAGATCTTTTTTTTTTTTCTCTTACAGTATGGTGAACACCAATCATGATCCCTAGAAAT TTCTTCTTCACCATACTCATCTGTGCTTTCAATGTCTGTGCTACTTTCACAGCAGTAGCCACTGCAAGCCCAGACTG TATAGGACCATTTGCTTCCTATGCACTTTTTGCCTTCGTTACTTGCATCTGCGTGTGTAGCATAGTCTGCCTGGTTA TTAATTTTTTCCAACTGGTAGACTGGATCTTTGTACGAATTGCCTACCTACGTCACCATCCCGAATACCGCAATCAA AATGTTGCGGCACTTCTTAGGCTTATTTAAAACCATGCAGGCTATGCTACCAGTCATTTTAATTCTGCTACTACCCT GCATTGCCCTAGCTTCCACCGCCACTCGCGCTACACCTGAACAACTTAGAAAATGCAAATTTCAACAACCATGGTCA TTTCTTGATTGCTACCATGAAAAATCTGATTTCCCCACATACTGGATAGTGATTGTTGGAATAATTAACATACTTTC ATGTACCTTTTTCTCAATCACAATATACCCCACATTTAATTTTGGGTGGAATTCTCCCAATGCACTGGGTTACCCAC AAGAACCAGATGAACATATCCCACTACAACACATACAACAACCACTAGCACTGGTAGAGTATGAAAATGAGCCACAA CCTTCACTACCTCCTGCCATTAGTTACTTCAACCTAACCGGCGGAGATGACTGAAATACTCACCACCTCCAATTCCG CCGAGGATCTGCTTGATATGGACGGCCGCGTCTCAGAACAGCGACTCGCCCAACTACGCATCCGCCAGCAGCAGGAA CGCGTGACCAAAGAGCTCAGAGATGTCATCCAAATTCACCAATGCAAAAAAGGCATATTTTGCTTGGTAAAACAAGC CAAGATATCCTACGAGATCACCGCTACTGACCATCGCCTCTCTTACGAACTTGGCCCCCAACGACAAAAATTTACAT GCATGGTGGGAATCACCCCTATAGTTATCACTCAGCAAAGTGGAGATACTAAGGGGTGCATTCACTGCTCTTGCGAT TCCATCGAGTGCACCTACACCCTGCTAAAGACCCTATGCGGCCTAAGAGACCTGCTACCCATGAATTAAAAATTAAT AAAAAATCACTTACTTGAAATCAGCAATAAGGTCTCTGTTGAAATTTTCTCCCAGCAGCACCTCACTTCCCTCTTCC CAACTCTGGTATTCTAAACCCCGTTCAGCGGCATACTTTCTCCATACTTTAAAGGGGATGTCAAATTTTAACTCCTG TCCTGTACCCACAATCTTCATGTCTTTCTTCCCAGATGACCAAGAGAGTCCGGCTCAGTGATTCCTTCAACCCTGTC TACCCCTATGAAGATGAAAGCACCTCCCAACACCCCTTTATAAACCCAGGGTTTATTTCCCCAAATGGCTTTACACA AAGCCCAGACGGAGTTCTTACTTTAAAATGTTTAACCCCACTAACAACCACAGGCGGGTCTCTACAGTTAAAAGTGG GAGGGGGTCTTACAATAGATGACACCGACGGTTTTTTGAAAGAAAACATAAGTGCCACCACACCACTCGTTAAGACT GGTCACTCTATAGGTTTGTCGCTAGGACCCGGATTAGGAACAAATGAAAACAAACTTTGTGCCAAATTGGGAGAAGG ACTTACATTCAATTCCAACAACATTTGCATTAATGACAATATTAACACCCTATGGACAGGAGTTAACCCCACCAGAG CCAACTGTCAAATAATGGCCTCCAGTGAATCTAATGATTGCAAATTAATTCTAACACTAGTTAAAACTGGAGCCCTC GTCACTGCATTTGTTTATGTTATAGGAGTATCTAACGATTTTAATATGCTAACTACACATAAAAATATAAATTTCAC TGCAGAGCTGTTTTTTGATTCTACTGGTAATTTATTAACTAGCCTTTCATCCCTAAAAACTCCACTTAATCATAAAT CAGGGCAAAACATGGCTACTGGTGCCCTTACTAATGCTAAAGGTTTCATGCCCAGCACAACTGCCTATCCTTTCAAT GTTAATTCCAGAGAAAAAGAAAACTACATTTACGGAACTTGTTACTACACAGCTAGTGATCACACTGCTTTTCCCAT TGACATATCTGTCATGCTTAACCAAAGAGCATTAAATAATGAGACATCATATTGTATTCGTGTAACTTGGTCCTGGA ATACAGGAGTTGCCCCAGAAGTGCAAACCTCTGCTACTACCCTAGTCACCTCTCCATTTACCTTTTACTACATTAGA GAAGACGACTGACAAATAAAGTTTAACTTGTTTATTTAAAATCAATTCATAAAATTCGAGTAGTTATTTTGCCTCCC CCTTCCCATTTAACAGAATACACCAATCTCTCCCCACGCACAGCTTTAAACATTTGGATACCATTACAGATAGACAT AGTTTTAGATTCCACATTCCAAACAGTTTCAAAGCGAGCCAATCTGGGGTCAGTGATACATAAAAATGCATCGGGAT AGTCTTTTAAAGCGCTTTCACAGTCCAACTGCTGCGGATGCGACTCCGGAGTCTGGATCACGGTCATCTGGAAGAAG AACGATGGGAATCATAATCCGAAAACGGAATCGGGCGATTGTGTCTCATCAAACCCACAAGCAGCCGCTGTCTGCGT CGCTCCGTGCGACTGCTGTTTATAGGATCGGGATCCACAGTGTCCTGAAGCATGATTTTAATAGCCCTTAACATTAA CTTTCTGGTGCGGTGCGCGCAGCAACGCATTCTGATTTCACTTAGATTACTACAGTAGGTACAGCACATTATCACAA TATTGTTTAATAAACCATAATTAAAAGCGCTCCAGCCAAAACTCATATCAGATATAATCGCCCCTGCATGACCATCA TACCAAATTTTAATATAAATTAAATGTCGTTCCCTCAAAAACACACTACCCACATACATAATCTCTTTTGGCATGTG CATATTAACAATCTGTCTGTACCATGGACAACGTTGGTTAATCATGCAACCCAATATAACCTTCCGAAACCACACTG CCAACACCGCTCCCCCAGCCATGCATTGAAGTGAACCCTGCCGATTACAATGACAATGAAGAACCCAATTCTCTCGA CCATGAATCACTTGAGAATAAAAAATATCTATAGTAGCACAACAAAGACATAAATGCATGCATCTTCTCATAATTCT TAACTCCTCGGGATTTAGAAACATATCCCAAGGAATGGGAAACTCTTGCAGAACAGTAAAGCTGGCAGAACAAGGAA GACCACGAACACAACTTACACTATGCATAGTCATAGTATCACAATCTGGCAACAGCGGGTGGTCTTCAGTCATAGAA GCTCGGGTTTCATTTTCCTCACATCGTGGTAACTGGGCTCTGGTGTAAGGGTGATGTCTGGCGCATGATGTCGAGCG TGCGCGCAACCTTGTCATAATGGAGTTGCTTCCTGACATTCTCGTATTTTGTATAGCAAAACGCGGCCCTGGCACAA CACACTCTTCTTCGTCTTCTATCCTGCCGCTTAGTGTGTTCCGTCTGATAATTCAAGTACAGCCACACTCTTAAGTT GGTCAAAAGAATGCTGGCTTCAGTTGTAATCAAAACTCCATCATATTTAATTGTTCTAAGGAAATCATCCACGGTAG CATATGCAAATCCCAACCAAGCAATGCAACTGGATTGCGTTTCAAGCAGCAGAGGAGAGGGAAGAGACGGAAGAATC ATGTTAATTTTTATTCCAAACGATCTCGCAGTACTTCAAATTGTAGATCGCGCAGATGGCATCTATCGCCCCCACTG TGTTGGTGAAAAAGCACAGCTAAATCAAAAGAAATGCGATTTTCAAGGTGCTCAACGGTGGCTTCCAACAAAGCCTC CACGCGCACATCCAAAAACAAAAGAATACCAAAAGAAGGAGCATTTTCTAACTCCTCAAACATCATATTACATTCCT GCACCATTCCCAGATAATTTTCAGCTTTCCAGCCTTGAATTATTCGTGTCAGTTCTTGTGGTAAATCCAAACCACAC ATTACAAACAGGTCCCGGAGGGCGCCCTCCACCACCATTCTTAAACACACCCTCATAATGTCAAAATATCTTGCTCC TGTGTCACCTGTAGCAAATTAAGAATGGCATCATCAATTGACATGCCCTTGGCTCTAAGTTCTTCTCTAAGTTCTAG TTGTAAATACTCTTTCATATTATCACCAAACTGCTTAGCCAGAAGCCCCCCGGGAACAATAGCAGGGGACGCTACAG TGCAGTACAAGCGCAGACCTCCCCAATTGGCTCCAGCAAAAACAAGATTAGAATAAGCATACTGGGAACCACCAGTA ATATCATCAAAGTTGCTGGAAATATAATCAGGCAGAGTTTCTTGTAAAAATTGAATAAAAGAAAAATTTTCCAAAGA AACATTCAAAACCTCTGGGATGCAAATGCAATAGGTTACCGCGCTGCGCTCCAACATTATTAGTTTTGAATTAGTCT GTAAAATAAAAGAAACAAGCGTCATATCATAGTAGCCTGTCGAACAGGTGGATAAATCAGTCTTTCCATTACAAGAC AAGCCACAGGGTCTCCAGCTCGACCCTCGTAAAACCTGTCATCGTGATTAAACAACAGCACCGAAAGTTCCTCGCGG TGGCCAGCATGAATAATTCTTGATGAAGCATATAATCCAGACATGTTAGCATCAGTTAAAGAAAAAAAACAGCCAAC ATAGCCTCTGGGTATAATTATGCTTAATCTTAAGTATAGCAAAGCCACCCCTCGCGGATACAAAGTAAAAGGCACAG GAGAATAAAAAATATAATTATTTCTCTGCTGCTGTTCAGGCAACGTTGCCCCCGGTCCCTCTAAATACACATACAAA GCCTCATCAGCCATGGCTTACCAGACAAAGTATAGCGGGCGCACAAAGCACAAGCTCTAAAGAAGCTCTAAAGACGC TCTCCAACCTCTCCACAATATATACACAAGCCCTAAACTGACGTAATGGGAGTAAAGTGTAAAAAATCCCGCCAAGC CCAACACACACCCCGAAACTGCGTCAGCAGGGAAAAGTACAGTTTCACTTCCGCAAACCCAACAAGCGTAGCTTCCT CTTTCTCACGGTACGTCACATCCGATTAACTTGCAACGTCATTTTCCCACGGCCGCCCCGCCCATTTTAGCCGTTAA CCCCACAGCCAATCACCACACAGCGCGCACTTTTTTAAATTACCTCATTTACATATTGGCACCATTCCATCTATAAG GTATATTATATAGAGAG

GenBank Accession No. AP_000564 MTKRVRLSDSFNPVYPYEDESTSQHPFINPGFISPNGFTQSPDGVLTLKCLTPLTTTGGSLQLKVGGGLTIDDTDGF LKENISATTPLVKTGHSIGLSLGPGLGTNENKLCAKLGEGLTFNSNNICINDNINTLWTGVNPTRANCQIMASSESN DCKLILTLVKTGALVTAFVYVIGVSNDENMLTTHKNINFTAELFFDSTGNLLTSLSSLKTPLNHKSGONMATGALTN AKGFMPSTTAYPFNVNSREKENYIYGTCYYTASDHTAFPIDISVMLNQRALNNETSYCIRVTWSWNTGVAPEVQTSA TTLVTSPFTFYYIREDD

GenBank Accession No. AP_000543 MRRRAVLGGAVVYPEGPPPSYESVMQQQAAMIQPPLEVPFVPPRYLAPTEGRNSIRYSELSPLYDTTKLYLVDNKSA DIASLNYQNDHSNFLTTVVONNDFTPTEASTQTINFDERSRWGGHLKTIMHTNMPNVNEYMFSNKFKARVMVSRKAP EGVTVNDTYDHKEDILKYEWFEFILPEGNFSATMTIDLMNNAIIDNYLEIGRONGVLESDIGVKFDTRNFRLGWDPE TKLIMPGVYTYEAFHPDIVLLPGCGVDFTESRLSNLLGIRKRHPFQEGFKIMYEDLEGGNIPALLDVTAYEESKKDT TTETTTLAVAEETSEDDNITRGDTYITEKHKREAAAAEVKKELKIQPLEKDSKSRSYNVLEDKINTAYRSWYLSYNY GNPKKGIRSWTLLTTSDVTCGAEQVYWSLPDMMQDPVTFRSTRQVNNYPVVGAELMPVFSKSFYNEQAVYSQQLRQA TSLTHVENREPENQILIRPPAPTITTVSENVPALTDHGTLPLRSSIRGVQRVTVTDARRRTCPYVYKALGIVAPRVL SSRTF

GenBank Accession No. AP_000548 MATPSMMPQWAYMHIAGODASEYLSPGLVQFARATDTYFSMGNKERNPTVAPTHDVTTDRSQRLMLRFVPVDREDNT YSYKVRYTLAVGDNRVLDMASTFFDIRGVLDRGPSFKPYSGTAYNSLAPKGAPNTSQWIVTTGEDNATTYTFGIAST KGDNITKEGLEIGKDITADNKPIYADKTYQPEPQVGEESWTDIDGTNEKFGGRALKPATKMKPCYGSFARPTNIKGG QAKNRKVTPTEGDVEAEEPDIDMEFFDGREAADAFSPEIVLYTENVNLETPDSHVVYKPGTSDGNSHANLGQQAMPN RPNYIGFRDNFVGLMYYNSTGNMGVLAGQASQLNAVVDLQDRNTELSYQLLLDSLGDRSRYFSMWNQAVDSYDPDVR IIENHGVEDELPNYCFPLDGIGPGNKYQGIKPRDTAWEKDTKVSTANEIAIGNNLAMEINIQANLWRSFLYSNVALY LPDVYKYTPTNITLPANTNTYEYMNGRVVSPSLVDSYINIGARWSLDPMDNVNPFNHHRNAGLRYRSMLLGNGRYVP FHIQVPQKFFAVKNLLLLPGSYTYEWNFRKDVNMVLQSSLGNDLRTDGANISFTSINLYATFFPMAHNTASTLEAML RNDTNDQSFNDYLSAANMLYPIPANATNIPISIPSRNWAAFRGWSFTRLKTKETPSLGSGFDPYFVYSGSIPYLDGT FYLNHTFKKVSIMFDSSVSWPGNDRLLSPNEFEIKRTVDGEGYNVAQCNMTKDWFLVOMLANYNIGYOGFYIPEGYK DRMYSFFRNFQPMSRQVVDEVNYTDYKAVTLPYQHNNSGFVGYLAPTMRQGEPYPANYPYPLIGTTAVKSVTOKKFL CDRTMWRIPFSSNFMSMGALTDLGQNLLYANSAHALDMTFEVDPMDEPTLLYLLFEVFDVVRVHQPHRGVIEAVYLR TPFSAGNATT

GenBank Accession No. NC_011202 (SEQ ID NO: 211) CATCATCAATAATATACCTTATAGATGGAATGGTGCCAATATGTAAATGAGGTGATTTTAAAAAGTGTGGATCGTGT GGTGATTGGCTGTGGGGTTAACGGCTAAAAGGGGCGGTGCGACCGTGGGAAAATGACGTTTTGTGGGGGTGGAGTTT TTTTGCAAGTTGTCGCGGGAAATGTGACGCATAAAAAGGCTTTTTTCTCACGGAACTACTTAGTTTTCCCACGGTAT TTAACAGGAAATGAGGTAGTTTTGACCGGATGCAAGTGAAAATTGTTGATTTTCGCGCGAAAACTGAATGAGGAAGT GTTTTTCTGAATAATGTGGTATTTATGGCAGGGTGGAGTATTTGTTCAGGGCCAGGTAGACTTTGACCCATTACGTG GAGGTTTCGATTACCGTGTTTTTTACCTGAATTTCCGCGTACCGTGTCAAAGTCTTCTGTTTTTACGTAGGTGTCAG CTGATCGCTAGGGTATTTATACCTCAGGGTTTGTGTCAAGAGGCCACTCTTGAGTGCCAGCGAGAAGAGTTTTCTCC TCTGCGCCGGCAGTTTAATAATAAAAAAATGAGAGATTTGCGATTTCTGCCTCAGGAAATAATCTCTGCTGAGACTG GAAATGAAATATTGGAGCTTGTGGTGCACGCCCTGATGGGAGACGATCCGGAGCCACCTGTGCAGCTTTTTGAGCCT CCTACGCTTCAGGAACTGTATGATTTAGAGGTAGAGGGATCGGAGGATTCTAATGAGGAAGCTGTAAATGGCTTTTT TACCGATTCTATGCTTTTAGCTGCTAATGAAGGGTTAGAATTAGATCCGCCTTTGGACACTTTTGATACTCCAGGGG TAATTGTGGAAAGCGGTACAGGTGTAAGAAAATTACCTGATTTGAGTTCCGTGGACTGTGATTTGCACTGCTATGAA GACGGGTTTCCTCCGAGTGATGAGGAGGACCATGAAAAGGAGCAGTCCATGCAGACTGCAGCGGGTGAGGGAGTGAA GGCTGCCAATGTTGGTTTTCAGTTGGATTGCCCGGAGCTTCCTGGACATGGCTGTAAGTCTTGTGAATTTCACAGGA AAAATACTGGAGTAAAGGAACTGTTATGTTCGCTTTGTTATATGAGAACGCACTGCCACTTTATTTACAGTAAGTGT GTTTAAGTTAAAATTTAAAGGAATATGCTGTTTTTCACATGTATATTGAGTGTGAGTTTTGTGCTTCTTATTATAGG TCCTGTGTCTGATGCTGATGAATCACCATCTCCTGATTCTACTACCTCACCTCCTGAGATTCAAGCACCTGTTCCTG TGGACGTGCGCAAGCCCATTCCTGTGAAGCTTAAGCCTGGGAAACGTCCAGCAGTGGAAAAACTTGAGGACTTGTTA CAGGGTGGGGACGGACCTTTGGACTTGAGTACACGGAAACGTCCAAGACAATAAGTGTTCCATATCCGTGTTTACTT AAGGTGACGTCAATATTTGTGTGACAGTGCAATGTAATAAAAATATGTTAACTGTTCACTGGTTTTTATTGCTTTTT GGGCGGGGACTCAGGTATATAAGTAGAAGCAGACCTGTGTGGTTAGCTCATAGGAGCTGGCTTTCATCCATGGAGGT TTGGGCCATTTTGGAAGACCTTAGGAAGACTAGGCAACTGTTAGAGAACGCTTCGGACGGAGTCTCCGGTTTTTGGA GATTCTGGTTCGCTAGTGAATTAGCTAGGGTAGTTTTTAGGATAAAACAGGACTATAACCAAGAATTTGAAAAGTTG TTGGTAGATTGCCCAGGACTTTTTGAAGCTCTTAATTTGGGCCATCAGGTTCACTTTAAAGAAAAAGTTTTATCAGT TTTAGACTTTTCAACCCCAGGTAGAACTGCTGCTGCTGTGGCTTTTCTTACTTTTATATTAGATAAATGGATCCCGC AGACTCATTTCAGCAGGGGATACGTTTTGGATTTCATAGCCACAGCATTGTGGAGAACATGGAAGGTTCGCAAGATG AGGACAATCTTAGGTTACTGGCCAGTGCAGCCTTTGGGTGTAGCGGGAATCCTGAGGCATCCACCGGTCATGCCAGC GGTTCTGGAGGAGGAACAGCAAGAGGACAACCCGAGAGCCGGCCTGGACCCTCCAGTGGAGGAGGCGGAGTAGCTGA CTTGTCTCCTGAACTGCAACGGGTGCTTACTGGATCTACGTCCACTGGACGGGATAGGGGCGTTAAGAGGGAGAGGG CATCTAGTGGTACTGATGCTAGATCTGAGTTGGCTTTAAGTTTAATGAGTCGCAGACGTCCTGAAACCATTTGGTGG CATGAGGTTCAGAAAGAGGGAAGGGATGAAGTTTCTGTATTGCAGGAGAAATATTCACTGGAACAGGTGAAAACATG TTGGTTGGAGCCTGAGGATGATTGGGAGGTGGCCATTAAAAATTATGCCAAGATAGCTTTGAGGCCTGATAAACAGT ATAAGATTACTAGACGGATTAATATCCGGAATGCTTGTTACATATCTGGAAATGGGGCTGAGGTGGTAATAGATACT CAAGACAAGGCAGTTATTAGATGCTGCATGATGGATATGTGGCCTGGGGTAGTCGGTATGGAAGCAGTAACTTTTGT AAATGTTAAGTTTAGGGGAGATGGTTATAATGGAATAGTGTTTATGGCCAATACCAAACTTATATTGCATGGTTGTA GCTTTTTTGGTTTCAACAATACCTGTGTAGATGCCTGGGGACAGGTTAGTGTACGGGGATGTAGTTTCTATGCGTGT TGGATTGCCACAGCTGGCAGAACCAAGAGTCAATTGTCTCTGAAGAAATGCATATTTCAAAGATGTAACCTGGGCAT TCTGAATGAAGGCGAAGCAAGGGTCCGCCACTGCGCTTCTACAGATACTGGATGTTTTATTTTGATTAAGGGAAATG CCAGCGTAAAGCATAACATGATTTGCGGTGCTTCCGATGAGAGGCCTTATCAAATGCTCACTTGTGCTGGTGGGCAT TGTAATATGCTGGCTACTGTGCATATTGTTTCCCATCAACGCAAAAAATGGCCTGTTTTTGATCACAATGTGATGAC GAAGTGTACCATGCATGCAGGTGGGCGTAGAGGAATGTTTATGCCTTACCAGTGTAACATGAATCATGTGAAAGTGT TGTTGGAACCAGATGCCTTTTCCAGAATGAGCCTAACAGGAATTTTTGACATGAACATGCAAATCTGGAAGATCCTG AGGTATGATGATACGAGATCGAGGGTACGCGCATGCGAATGCGGAGGCAAGCATGCCAGGTTCCAGCCGGTGTGTGT AGATGTGACTGAAGATCTCAGACCGGATCATTTGGTTATTGCCCGCACTGGAGCAGAGTTCGGATCCAGTGGAGAAG AAACTGACTAAGGTGAGTATTGGGAAAACTTTGGGGTGGGATTTTCAGATGGACAGATTGAGTAAAAATTTGTTTTT TCTGTCTTGCAGCTGTCATGAGTGGAAACGCTTCTTTTAAGGGGGGAGTCTTCAGCCCTTATCTGACAGGGCGTCTC CCATCCTGGGCAGGAGTTCGTCAGAATGTTATGGGATCTACTGTGGATGGAAGACCCGTCCAACCCGCCAATTCTTC AACGCTGACCTATGCTACTTTAAGTTCTTCACCTTTGGACGCAGCTGCAGCTGCCGCCGCCGCTTCTGTTGCCGCTA ACACTGTGCTTGGAATGGGTTACTATGGAAGCATCATGGCTAATTCCACTTCCTCTAATAACCCTTCTACCCTGACT CAGGACAAGTTACTTGTCCTTTTGGCCCAGCTGGAGGCTTTGACCCAACGTCTGGGTGAACTTTCTCAGCAGGTGGT CGAGTTGCGAGTACAAACTGAGTCTGCTGTCGGCACGGCAAAGTCTAAATAAAAAAATCCCAGAATCAATGAATAAA TAAACAAGCTTGTTGTTGATTTAAAATCAAGTGTTTTTATTTCATTTTTCGCGCACGGTATGCCCTAGACCACCGAT CTCTATCATTGAGAACTCGGTGGATTTTTTCCAGGATCCTATAGAGGTGGGATTGAATGTTTAGATACATGGGCATT AGGCCGTCTTTGGGGTGGAGATAGCTCCATTGAAGGGATTCATGCTCCGGGGTAGTGTTGTAAATCACCCAGTCATA ACAAGGTCGCAGTGCATGGTGTTGCACAATATCTTTTAGAAGTAGGCTGATTGCCACAGATAAGCCCTTGGTGTAGG TGTTTACAAACCGGTTGAGCTGGGATGGGTGCATTCGGGGTGAAATTATGTGCATTTTGGATTGGATTTTTAAGTTG GCAATATTGCCGCCAAGATCCCGTCTTGGGTTCATGTTATGAAGGACCACCAAGACGGTGTATCCGGTACATTTAGG AAATTTATCGTGCAGCTTGGATGGAAAAGCGTGGAAAAATTTGGAGACACCCTTGTGTCCTCCAAGATTTTCCATGC ACTCATCCATGATAATAGCAATGGGGCCGTGGGCAGCGGCGCGGGCAAACACGTTCCGTGGGTCTGACACATCATAG TTATGTTCCTGAGTTAAATCATCATAAGCCATTTTAATGAATTTGGGGCGGAGAGTACCAGATTGGGGTATGAATGT TCCTTCGGGCCCCGGAGCATAGTTCCCCTCACAGATTTGCATTTCCCAAGCTTTCAGTTCCGAGGGTGGAATCATGT CCACCTGGGGGGCTATGAAAAACACCGTTTCTGGGGCGGGGGTGATTAATTGTGATGATAGCAAATTTCTGAGCAAT TGAGATTTGCCACATCCGGTGGGGCCATAAATGATTCCGATTACGGGTTGCAGGTGGTAGTTTAGGGAACGGCAACT GCCGTCTTCTCGAAGCAAGGGGGCCACCTCGTTCATCATTTCCCTTACATGCATATTTTCCCGCACCAAATCCATTA GGAGGCGCTCTCCTCCTAGTGATAGAAGTTCTTGTAGTGAGGAAAAGTTTTTCAGCGGTTTCAGACCGTCAGCCATG GGCATTTTGGAGAGAGTTTGCTGCAAAAGTTCTAGTCTGTTCCACAGTTCAGTGATGTGTTCTATGGCATCTCGATC CAGCAGACCTCCTCGTTTCGCGGGTTTGGACGGCTCCTGGAATAGGGTATGAGACGATGGGCGTCCAGCGCTGCCAG GGTTCGGTCCTTCCAGGGTCTCAGTGTTCGAGTCAGGGTTGTTTCCGTCACAGTGAAGGGGTGTGCGCCTGCTTGGG CGCTTGCCAGGGTGCGCTTCAGACTCATCCTGCTGGTCGAAAACTTCTGTCGCTTGGCGCCCTGTATGTCGGCCAAG TAGCAGTTTACCATGAGTTCGTAGTTGAGCGCCTCGGCTGCGTGGCCTTTGGCGCGGAGCTTACCTTTGGAAGTTTT CTTGCATACCGGGCAGTATAGGCATTTCAGCGCATACAACTTGGGCGCAAGGAAAACGGATTCTGGGGAGTATGCAT CTGCGCCGCAGGAGGCGCAAACAGTTTCACATTCCACCAGCCAGGTTAAATCCGGTTCATTGGGGTCAAAAACAAGT TTTCCGCCATATTTTTTGATGCGTTTCTTACCTTTGGTCTCCATGAGTTCGTGTCCTCGTTGAGTGACAAACAGGCT GTCCGTGTCCCCGTAGACTGATTTTACAGGCCTCTTCTCCAGTGGAGTGCCTCGGTCTTCTTCGTACAGGAACTCTG ACCACTCTGATACAAAGGCGCGCGTCCAGGCCAGCACAAAGGAGGCTATGTGGGAGGGGTAGCGATCGTTGTCAACC AGGGGGTCCACCTTTTCCAAAGTATGCAAACACATGTCACCCTCTTCAACATCCAGGAATGTGATTGGCTTGTAGGT GTATTTCACGTGACCTGGGGTCCCCGCTGGGGGGGTATAAAAGGGGGCGGTTCTTTGCTCTTCCTCACTGTCTTCCG GATCGCTGTCCAGGAACGTCAGCTGTTGGGGTAGGTATTCCCTCTCGAAGGCGGGCATGACCTCTGCACTCAGGTTG TCAGTTTCTAAGAACGAGGAGGATTTGATATTGACAGTGCCGGTTGAGATGCCTTTCATGAGGTTTTCGTCCATTTG GTCAGAAAACACAATTTTTTTATTGTCAAGTTTGGTGGCAAATGATCCATACAGGGCGTTGGATAAAAGTTTGGCAA TGGATCGCATGGTTTGGTTCTTTTCCTTGTCCGCGCGCTCTTTGGCGGCGATGTTGAGTTGGACATACTCGCGTGCC AGGCACTTCCATTCGGGGAAGATAGTTGTTAATTCATCTGGCACGATTCTCACTTGCCACCCTCGATTATGCAAGGT AATTAAATCCACACTGGTGGCCACCTCGCCTCGAAGGGGTTCATTGGTCCAACAGAGCCTACCTCCTTTCCTAGAAC AGAAAGGGGGAAGTGGGTCTAGCATAAGTTCATCGGGAGGGTCTGCATCCATGGTAAAGATTCCCGGAAGTAAATCC TTATCAAAATAGCTGATGGGAGTGGGGTCATCTAAGGCCATTTGCCATTCTCGAGCTGCCAGTGCGCGCTCATATGG GTTAAGGGGACTGCCCCATGGCATGGGATGGGTGAGTGCAGAGGCATACATGCCACAGATGTCATAGACGTAGATGG GATCCTCAAAGATGCCTATGTAGGTTGGATAGCATCGCCCCCCTCTGATACTTGCTCGCACATAGTCATATAGTTCA TGTGATGGCGCTAGCAGCCCCGGACCCAAGTTGGTGCGATTGGGTTTTTCTGTTCTGTAGACGATCTGGCGAAAGAT GGCGTGAGAATTGGAAGAGATGGTGGGTCTTTGAAAAATGTTGAAATGGGCATGAGGTAGACCTACAGAGTCTCTGA CAAAGTGGGCATAAGATTCTTGAAGCTTGGTTACCAGTTCGGCGGTGACAAGTACGTCTAGGGCGCAGTAGTCAAGT GTTTCTTGAATGATGTCATAACCTGGTTGGTTTTTCTTTTCCCACAGTTCGCGGTTGAGAAGGTATTCTTCGCGATC CTTCCAGTACTCTTCTAGCGGAAACCCGTCTTTGTCTGCACGGTAAGATCCTAGCATGTAGAACTGATTAACTGCCT TGTAAGGGCAGCAGCCCTTCTCTACGGGTAGAGAGTATGCTTGAGCAGCTTTTCGTAGCGAAGCGTGAGTAAGGGCA AAGGTGTCTCTGACCATGACTTTGAGAAATTGGTATTTGAAGTCGATGTCGTCACAGGCTCCCTGTTCCCAGAGTTG GAAGTCTACCCGTTTCTTGTAGGCGGGGTTGGGCAAAGCGAAAGTAACATCATTGAAGAGAATCTTACCGGCTCTGG GCATAAAATTGCGAGTGATGCGAAAAGGCTGTGGTACTTCCGCTCGATTGTTGATCACCTGGGCAGCTAGGACGATC TCGTCGAAACCGTTGATGTTGTGTCCTACGATGTATAATTCTATGAAACGCGGCGTGCCTCTGACGTGAGGTAGCTT ACTGAGCTCATCAAAGGTTAGGTCTGTGGGGTCAGATAAGGCGTAGTGTTCGAGAGCCCATTCGTGCAGGTGAGGAT TTGCATGTAGGAATGATGACCAAAGATCTACCGCCAGTGCTGTTTGTAACTGGTCCCGATACTGACGAAAATGCCGG CCAATTGCCATTTTTTCTGGAGTGACACAGTAGAAGGTTCTGGGGTCTTGTTGCCATCGATCCCACTTGAGTTTAAT GGCTAGATCGTGGGCCATGTTGACGAGACGCTCTTCTCCTGAGAGTTTCATGACCAGCATGAAAGGAACTAGTTGTT TGCCAAAGGATCCCATCCAGGTGTAAGTTTCCACATCGTAGGTCAGGAAGAGTCTTTCTGTGCGAGGATGAGAGCCG ATCGGGAAGAACTGGATTTCCTGCCACCAGTTGGAGGATTGGCTGTTGATGTGATGGAAGTAGAAGTTTCTGCGGCG CGCCGAGCATTCGTGTTTGTGCTTGTACAGACGGCCGCAGTAGTCGCAGCGTTGCACGGGTTGTATCTCGTGAATGA GTTGTACCTGGCTTCCCTTGACGAGAAATTTCAGTGGGAAGCCGAGGCCTGGCGATTGTATCTCGTGCTCTTCTATA TTCGCTGTATCGGCCTGTTCATCTTCTGTTTCGATGGTGGTCATGCTGACGAGCCCCCGCGGGAGGCAAGTCCAGAC CTCGGCGCGGGAGGGGCGGAGCTGAAGGACGAGAGCGCGCAGGCTGGAGCTGTCCAGAGTCCTGAGACGCTGCGGAC TCAGGTTAGTAGGTAGGGACAGAAGATTAACTTGCATGATCTTTTCCAGGGCGTGCGGGAGGTTCAGATGGTACTTG ATTTCCACAGGTTCGTTTGTAGAGACGTCAATGGCTTGCAGGGTTCCGTGTCCTTTGGGCGCCACTACCGTACCTTT GTTTTTTCTTTTGATCGGTGGTGGCTCTCTTGCTTCTTGCATGCTCAGAAGCGGTGACGGGGACGCGCGCCGGGCGG CAGCGGTTGTTCCGGACCCGAGGGCATGGCTGGTAGTGGCACGTCGGCGCCGCGCACGGGCAGGTTCTGGTACTGCG CTCTGAGAAGACTTGCGTGCGCCACCACGCGTCGATTGACGTCTTGTATCTGACGTCTCTGGGTGAAAGCTACCGGC CCCGTGAGCTTGAACCTGAAAGAGAGTTCAACAGAATCAATTTCGGTATCGTTAACGGCAGCTTGTCTCAGTATTTC TTGTACGTCACCAGAGTTGTCCTGGTAGGCGATCTCCGCCATGAACTGCTCGATTTCTTCCTCCTGAAGATCTCCGC GACCCGCTCTTTCGACGGTGGCCGCGAGGTCATTGGAGATACGGCCCATGAGTTGGGAGAATGCATTCATGCCCGCC TCGTTCCAGACGCGGCTGTAAACCACGGCCCCCTCGGAGTCTCTTGCGCGCATCACCACCTGAGCGAGGTTAAGCTC CACGTGTCTGGTGAAGACCGCATAGTTGCATAGGCGCTGAAAAAGGTAGTTGAGTGTGGTGGCAATGTGTTCGGCGA CGAAGAAATACATGATCCATCGTCTCAGCGGCATTTCGCTAACATCGCCCAGAGCTTCCAAGCGCTCCATGGCCTCG TAGAAGTCCACGGCAAAATTAAAAAACTGGGAGTTTCGCGCGGACACGGTCAATTCCTCCTCGAGAAGACGGATGAG TTCGGCTATGGTGGCCCGTACTTCGCGTTCGAAGGCTCCCGGGATCTCTTCTTCCTCTTCTATCTCTTCTTCCACTA ACATCTCTTCTTCGTCTTCAGGCGGGGGGGGAGGGGGCACGCGGCGACGTCGACGGCGCACGGGCAAACGGTCGATG AATCGTTCAATGACCTCTCCGCGGCGGCGGCGCATGGTTTCAGTGACGGCGCGGCCGTTCTCGCGCGGTCGCAGAGT AAAAACACCGCCGCGCATCTCCTTAAAGTGGTGACTGGGAGGTTCTCCGTTTGGGAGGGAGAGGGCGCTGATTATAC ATTTTATTAATTGGCCCGTAGGGACTGCACGCAGAGATCTGATCGTGTCAAGATCCACGGGATCTGAAAACCTTTCG ACGAAAGCGTCTAACCAGTCACAGTCACAAGGTAGGCTGAGTACGGCTTCTTGTGGGGGGGGGTGGTTATGTGTTCG GTCTGGGTCTTCTGTTTCTTCTTCATCTCGGGAAGGTGAGACGATGCTGCTGGTGATGAAATTAAAGTAGGCAGTTC TAAGACGGCGGATGGTGGCGAGGAGCACCAGGTCTTTGGGTCCGGCTTGCTGGATACGCAGGCGATTGGCCATTCCC CAAGCATTATCCTGACATCTAGCAAGATCTTTGTAGTAGTCTTGCATGAGCCGTTCTACGGGCACTTCTTCCTCACC CGTTCTGCCATGCATACGTGTGAGTCCAAATCCGCGCATTGGTTGTACCAGTGCCAAGTCAGCTACGACTCTTTCGG CGAGGATGGCTTGCTGTACTTGGGTAAGGGTGGCTTGAAAGTCATCAAAATCCACAAAGCGGTGGTAAGCTCCTGTA TTAATGGTGTAAGCACAGTTGGCCATGACTGACCAGTTAACTGTCTGGTGACCAGGGCGCACGAGCTCGGTGTATTT AAGGCGCGAATAGGCGCGGGTGTCAAAGATGTAATCGTTGCAGGTGCGCACCAGATACTGGTACCCTATAAGAAAAT GCGGCGGTGGTTGGCGGTAGAGAGGCCATCGTTCTGTAGCTGGAGCGCCAGGGGCGAGGTCTTCCAACATAAGGCGG TGATAGCCGTAGATGTACCTGGACATCCAGGTGATTCCTGCGGCGGTAGTAGAAGCCCGAGGAAACTCGCGTACGCG GTTCCAAATGTTGCGTAGCGGCATGAAGTAGTTCATTGTAGGCACGGTTTGACCAGTGAGGCGCGCGCAGTCATTGA TGCTCTATAGACACGGAGAAAATGAAAGCGTTCAGCGACTCGACTCCGTAGCCTGGAGGAACGTGAACGGGTTGGGT CGCGGTGTACCCCGGTTCGAGACTTGTACTCGAGCCGGCCGGAGCCGCGGCTAACGTGGTATTGGCACTCCCGTCTC GACCCAGCCTACAAAAATCCAGGATACGGAATCGAGTCGTTTTGCTGGTTTCCGAATGGCAGGGAAGTGAGTCCTAT TTTTTTTTTTTGCCGCTCAGATGCATCCCGTGCTGCGACAGATGCGCCCCCAACAACAGCCCCCCTCGCAGCAGCAG CAGCAGCAATCACAAAAGGCTGTCCCTGCAACTACTGCAACTGCCGCCGTGAGCGGTGCGGGACAGCCCGCCTATGA TCTGGACTTGGAAGAGGGCGAAGGACTGGCACGTCTAGGTGCGCCTTCACCCGAGCGGCATCCGCGAGTTCAACTGA AAAAAGATTCTCGCGAGGCGTATGTGCCCCAACAGAACCTATTTAGAGACAGAAGCGGCGAGGAGCCGGAGGAGATG CGAGCTTCCCGCTTTAACGCGGGTCGTGAGCTGCGTCACGGTTTGGACCGAAGACGAGTGTTGCGGGACGAGGATTT CGAAGTTGATGAAATGACAGGGATCAGTCCTGCCAGGGCACACGTGGCTGCAGCCAACCTTGTATCGGCTTACGAGC AGACAGTAAAGGAAGAGCGTAACTTCCAAAAGTCTTTTAATAATCATGTGCGAACCCTGATTGCCCGCGAAGAAGTT ACCCTTGGTTTGATGCATTTGTGGGATTTGATGGAAGCTATCATTCAGAACCCTACTAGCAAACCTCTGACCGCCCA GCTGTTTCTGGTGGTGCAACACAGCAGAGACAATGAGGCTTTCAGAGAGGCGCTGCTGAACATCACCGAACCCGAGG GGAGATGGTTGTATGATCTTATCAACATTCTACAGAGTATCATAGTGCAGGAGCGGAGCCTGGGCCTGGCCGAGAAG GTGGCTGCCATCAATTACTCGGTTTTGAGCTTGGGAAAATATTACGCTCGCAAAATCTACAAGACTCCATACGTTCC CATAGACAAGGAGGTGAAGATAGATGGGTTCTACATGCGCATGACGCTCAAGGTCTTGACCCTGAGCGATGATCTTG GGGTGTATCGCAATGACAGAATGCATCGCGCGGTTAGCGCCAGCAGGAGGCGCGAGTTAAGCGACAGGGAACTGATG CACAGTTTGCAAAGAGCTCTGACTGGAGCTGGAACCGAGGGTGAGAATTACTTCGACATGGGAGCTGACTTGCAGTG GCAGCCTAGTCGCAGGGCTCTGAGCGCCGCGACGGCAGGATGTGAGCTTCCTTACATAGAAGAGGCGGATGAAGGCG AGGAGGAAGAGGGCGAGTACTTGGAAGACTGATGGCACAACCCGTGTTTTTTGCTAGATGGAACAGCAAGCACCGGA TCCCGCAATGCGGGCGGCGCTGCAGAGCCAGCCGTCCGGCATTAACTCCTCGGACGATTGGACCCAGGCCATGCAAC GTATCATGGCGTTGACGACTCGCAACCCCGAAGCCTTTAGACAGCAACCCCAGGCCAACCGTCTATCGGCCATCATG GAAGCTGTAGTGCCTTCCCGCTCTAATCCCACTCATGAGAAGGTCCTGGCCATCGTGAACGCGTTGGTGGAGAACAA AGCTATTCGTCCAGATGAGGCCGGACTGGTATACAACGCTCTCTTAGAACGCGTGGCTCGCTACAACAGTAGCAATG TGCAAACCAATTTGGACCGTATGATAACAGATGTACGCGAAGCCGTGTCTCAGCGCGAAAGGTTCCAGCGTGATGCC AACCTGGGTTCGCTGGTGGCGTTAAATGCTTTCTTGAGTACTCAGCCTGCTAATGTGCCGCGTGGTCAACAGGATTA TACTAACTTTTTAAGCGCTTTGAGACTGATGGTATCAGAAGTACCTCAGAGCGAAGTGTATCAGTCCGGTCCTGATT ACTTCTTTCAGACTAGCAGACAGGGCTTGCAGACGGTAAATCTGAGCCAAGCTTTTAAAAACCTTAAAGGTTTGTGG GGAGTGCATGCCCCGGTAGGAGAAAGAGCAACCGTGTCTAGCTTGTTAACTCCGAACTCCCGCCTATTATTACTGTT GGTAGCTCCTTTCACCGACAGCGGTAGCATCGACCGTAATTCCTATTTGGGTTACCTACTAAACCTGTATCGCGAAG CCATAGGGCAAAGTCAGGTGGACGAGCAGACCTATCAAGAAATTACCCAAGTCAGTCGCGCTTTGGGACAGGAAGAC ACTGGCAGTTTGGAAGCCACTCTGAACTTCTTGCTTACCAATCGGTCTCAAAAGATCCCTCCTCAATATGCTCTTAC TGCGGAGGAGGAGAGGATCCTTAGATATGTGCAGCAGAGCGTGGGATTGTTTCTGATGCAAGAGGGGGCAACTCCGA CTGCAGCACTGGACATGACAGCGCGAAATATGGAGCCCAGCATGTATGCCAGTAACCGACCTTTCATTAACAAACTG CTGGACTACTTGCACAGAGCTGCCGCTATGAACTCTGATTATTTCACCAATGCCATCTTAAACCCGCACTGGCTGCC CCCACCTGGTTTCTACACGGGCGAATATGACATGCCCGACCCTAATGACGGATTTCTGTGGGACGACGTGGACAGCG ATGTTTTTTCACCTCTTTCTGATCATCGCACGTGGAAAAAGGAAGGCGGCGATAGAATGCATTCTTCTGCATCGCTG TCCGGGGTCATGGGTGCTACCGCGGCTGAGCCCGAGTCTGCAAGTCCTTTTCCTAGTCTACCCTTTTCTCTACACAG TGTACGTAGCAGCGAAGTGGGTAGAATAAGTCGCCCGAGTTTAATGGGCGAAGAGGAGTATCTAAACGATTCCTTGC TCAGACCGGCAAGAGAAAAAAATTTCCCAAACAATGGAATAGAAAGTTTGGTGGATAAAATGAGTAGATGGAAGACT TATGCTCAGGATCACAGAGACGAGCCTGGGATCATGGGGATTACAAGTAGAGCGAGCCGTAGACGCCAGCGCCATGA CAGACAGAGGGGTCTTGTGTGGGACGATGAGGATTCGGCCGATGATAGCAGCGTGCTGGACTTGGGTGGGAGAGGAA GGGGCAACCCGTTTGCTCATTTGCGCCCTCGCTTGGGTGGTATGTTGTAAAAAAAAATAAAAAAAAAACTCACCAAG GCCATGGCGACGAGCGTACGTTCGTTCTTCTTTATTATCTGTGTCTAGTATAATGAGGCGAGTCGTGCTAGGCGGAG CGGTGGTGTATCCGGAGGGTCCTCCTCCTTCGTACGAGAGCGTGATGCAGCAGCAGCAGGCGACGGCGGTGATGCAA TCCCCACTGGAGGCTCCCTTTGTGCCTCCGCGATACCTGGCACCTACGGAGGGCAGAAACAGCATTCGTTATTCGGA ACTGGCACCTCAGTACGATACCACCAGGTTGTATCTGGTGGACAACAAGTCGGCGGACATTGCTTCTCTGAACTATC AGAATGACCACAGCAACTTCTTGACCACGGTGGTGCAAAACAATGACTTTACCCCTACGGAAGCCAGCACCCAGACC ATTAACTTTGATGAACGATCGCGGTGGGGCGGTCAGCTAAAGACCATCATGCATACTAACATGCCAAACGTGAACGA GTATATGTTTAGTAACAAGTTCAAAGCGCGTGTGATGGTGTCCAGAAAACCTCCCGACGGTGCTGCAGTTGGGGATA CTTATGATCACAAGCAGGATATTTTGAAATATGAGTGGTTCGAGTTTACTTTGCCAGAAGGCAACTTTTCAGTTACT ATGACTATTGATTTGATGAACAATGCCATCATAGATAATTACTTGAAAGTGGGTAGACAGAATGGAGTGCTTGAAAG TGACATTGGTGTTAAGTTCGACACCAGGAACTTCAAGCTGGGATGGGATCCCGAAACCAAGTTGATCATGCCTGGAG TGTATACGTATGAAGCCTTCCATCCTGACATTGTCTTACTGCCTGGCTGCGGAGTGGATTTTACCGAGAGTCGTTTG AGCAACCTTCTTGGTATCAGAAAAAAACAGCCATTTCAAGAGGGTTTTAAGATTTTGTATGAAGATTTAGAAGGTGG TAATATTCCGGCCCTCTTGGATGTAGATGCCTATGAGAACAGTAAGAAAGAACAAAAAGCCAAAATAGAAGCTGCTA CAGCTGCTGCAGAAGCTAAGGCAAACATAGTTGCCAGCGACTCTACAAGGGTTGCTAACGCTGGAGAGGTCAGAGGA GACAATTTTGCGCCAACACCTGTTCCGACTGCAGAATCATTATTGGCCGATGTGTCTGAAGGAACGGACGTGAAACT CACTATTCAACCTGTAGAAAAAGATAGTAAGAATAGAAGCTATAATGTGTTGGAAGACAAAATCAACACAGCCTATC GCAGTTGGTATCTTTCGTACAATTATGGCGATCCCGAAAAAGGAGTGCGTTCCTGGACATTGCTCACCACCTCAGAT GTCACCTGCGGAGCAGAGCAGGTCTACTGGTCGCTTCCAGACATGATGAAGGATCCTGTCACTTTCCGCTCCACTAG ACAAGTCAGTAACTACCCTGTGGTGGGTGCAGAGCTTATGCCCGTCTTCTCAAAGAGCTTCTACAACGAACAAGCTG TGTACTCCCAGCAGCTCCGCCAGTCCACCTCGCTTACGCACGTCTTCAACCGCTTTCCTGAGAACCAGATTTTAATC CGTCCGCCGGCGCCCACCATTACCACCGTCAGTGAAAACGTTCCTGCTCTCACAGATCACGGGACCCTGCCGTTGCG CAGCAGTATCCGGGGAGTCCAACGTGTGACCGTTACTGACGCCAGACGCCGCACCTGTCCCTACGTGTACAAGGCAC TGGGCATAGTCGCACCGCGCGTCCTTTCAAGCCGCACTTTCTAAAAAAAAAAAAAATGTCCATTCTTATCTCGCCCA GTAATAACACCGGTTGGGGTCTGCGCGCTCCAAGCAAGATGTACGGAGGCGCACGCAAACGTTCTACCCAACATCCT GTCCGTGTTCGCGGACATTTTCGCGCTCCATGGGGCGCCCTCAAGGGCCGCACTCGCGTTCGAACCACCGTCGATGA TGTAATCGATCAGGTGGTTGCCGACGCCCGTAATTATACTCCTACTGCGCCTACATCTACTGTGGATGCAGTTATTG ACAGTGTAGTGGCTGACGCTCGCAACTATGCTCGACGTAAGAGCCGGCGAAGGCGCATTGCCAGACGCCACCGAGCT ACCACTGCCATGCGAGCCGCAAGAGCTCTGCTACGAAGAGCTAGACGCGTGGGGCGAAGAGCCATGCTTAGGGCGGC CAGACGTGCAGCTTCGGGCGCCAGCGCCGGCAGGTCCCGCAGGCAAGCAGCCGCTGTCGCAGCGGCGACTATTGCCG ACATGGCCCAATCGCGAAGAGGCAATGTATACTGGGTGCGTGACGCTGCCACCGGTCAACGTGTACCCGTGCGCACC CGTCCCCCTCGCACTTAGAAGATACTGAGCAGTCTCCGATGTTGTGTCCCAGCGGCGAGGATGTCCAAGCGCAAATA CAAGGAAGAAATGCTGCAGGTTATCGCACCTGAAGTCTACGGCCAACCGTTGAAGGATGAAAAAAAACCCCGCAAAA TCAAGCGGGTTAAAAAGGACAAAAAAGAAGAGGAAGATGGCGATGATGGGCTGGCGGAGTTTGTGCGCGAGTTTGCC CCACGGCGACGCGTGCAATGGCGTGGGCGCAAAGTTCGACATGTGTTGAGACCTGGAACTTCGGTGGTCTTTACACC CGGCGAGCGTTCAAGCGCTACTTTTAAGCGTTCCTATGATGAGGTGTACGGGGATGATGATATTCTTGAGCAGGCGG CTGACCGATTAGGCGAGTTTGCTTATGGCAAGCGTAGTAGAATAACTTCCAAGGATGAGACAGTGTCGATACCCTTG GATCATGGAAATCCCACCCCTAGTCTTAAACCGGTCACTTTGCAGCAAGTGTTACCCGTAACTCCGCGAACAGGTGT TAAACGCGAAGGTGAAGATTTGTATCCCACTATGCAACTGATGGTACCCAAACGCCAGAAGTTGGAGGACGTTTTGG AGAAAGTAAAAGTGGATCCAGATATTCAACCTGAGGTTAAAGTGAGACCCATTAAGCAGGTAGCGCCTGGTCTGGGG GTACAAACTGTAGACATTAAGATTCCCACTGAAAGTATGGAAGTGCAAACTGAACCCGCAAAGCCTACTGCCACCTC CACTGAAGTGCAAACGGATCCATGGATGCCCATGCCTATTACAACTGACGCCGCCGGTCCCACTCGAAGATCCCGAC GAAAGTACGGTCCAGCAAGTCTGTTGATGCCCAATTATGTTGTACACCCATCTATTATTCCTACTCCTGGTTACCGA GGCACTCGCTACTATCGCAGCCGAAACAGTACCTCCCGCCGTCGCCGCAAGACACCTGCAAATCGCAGTCGTCGCCG TAGACGCACAAGCAAACCGACTCCCGGCGCCCTGGTGCGGCAAGTGTACCGCAATGGTAGTGCGGAACCTTTGACAC TGCCGCGTGCGCGTTACCATCCGAGTATCATCACTTAATCAATGTTGCCGCTGCCTCCTTGCAGATATGGCCCTCAC TTGTCGCCTTCGCGTTCCCATCACTGGTTACCGAGGAAGAAACTCGCGCCGTAGAAGAGGGATGTTGGGACGCGGAA TGCGACGCTACAGGCGACGGCGTGCTATCCGCAAGCAATTGCGGGGTGGTTTTTTACCAGCCTTAATTCCAATTATC GCTGCTGCAATTGGCGCGATACCAGGCATAGCTTCCGTGGCGGTTCAGGCCTCGCAACGACATTGACATTGGAAAAA AACGTATAAATAAAAAAAAAAAAATACAATGGACTCTGACACTCCTGGTCCTGTGACTATGTTTTCTTAGAGATGGA AGACATCAATTTTTCATCCTTGGCTCCGCGACACGGCACGAAGCCGTACATGGGCACCTGGAGCGACATCGGCACGA GCCAACTGAACGGGGGCGCCTTCAATTGGAGCAGTATCTGGAGCGGGCTTAAAAATTTTGGCTCAACCATAAAAACA TACGGGAACAAAGCTTGGAACAGCAGTACAGGACAGGCGCTTAGAAATAAACTTAAAGACCAGAACTTCCAACAAAA AGTAGTCGATGGGATAGCTTCCGGCATCAATGGAGTGGTAGATTTGGCTAACCAGGCTGTGCAGAAAAAGATAAACA GTCGTTTGGACCCGCCGCCAGCAACCCCAGGTGAAATGCAAGTGGAGGAAGAAATTCCTCCGCCAGAAAAACGAGGC GACAAGCGTCCGCGTCCCGATTTGGAAGAGACGCTGGTGACGCGCGTAGATGAACCGCCTTCTTATGAGGAAGCAAC GAAGCTTGGAATGCCCACCACTAGACCGATAGCCCCAATGGCCACCGGGGTGATGAAACCTTCTCAGTTGCATCGAC CCGTCACCTTGGATTTGCCCCCTCCCCCTGCTGCTACTGCTGTACCCGCTTCTAAGCCTGTCGCTGCCCCGAAACCA GTCGCCGTAGCCAGGTCACGTCCCGGGGGCGCTCCTCGTCCAAATGCGCACTGGCAAAATACTCTGAACAGCATCGT GGGTCTAGGCGTGCAAAGTGTAAAACGCCGTCGCTGCTTTTAATTAAATATGGAGTAGCGCTTAACTTGCCTATCTG TGTATATGTGTCATTACACGCCGTCACAGCAGCAGAGGAAAAAAGGAAGAGGTCGTGCGTCGACGCTGAGTTACTTT CAAGATGGCCACCCCATCGATGCTGCCCCAATGGGCATACATGCACATCGCCGGACAGGATGCTTCGGAGTACCTGA GTCCGGGTCTGGTGCAGTTCGCCCGCGCCACAGACACCTACTTCAATCTGGGAAATAAGTTTAGAAATCCCACCGTA GCGCCGACCCACGATGTGACCACCGACCGTAGCCAGCGGCTCATGTTGCGCTTCGTGCCCGTTGACCGGGAGGACAA TACATACTCTTACAAAGTGCGGTACACCCTGGCCGTGGGCGACAACAGAGTGCTGGATATGGCCAGCACGTTCTTTG ACATTAGGGGTGTGTTGGACAGAGGTCCCAGTTTCAAACCCTATTCTGGTACGGCTTACAACTCCCTGGCTCCTAAA GGCGCTCCAAATACATCTCAGTGGATTGCAGAAGGTGTAAAAAATACAACTGGTGAGGAACACGTAACAGAAGAGGA AACCAATACTACTACTTACACTTTTGGCAATGCTCCTGTAAAAGCTGAAGCTGAAATTACAAAAGAAGGACTCCCAG TAGGTTTGGAAGTTTCAGATGAAGAAAGTAAACCGATTTATGCTGATAAAACATATCAGCCAGAACCTCAGCTGGGA GATGAAACTTGGACTGACCTTGATGGAAAAACCGAAAAGTATGGAGGCAGGGCTCTCAAACCCGATACTAAGATGAA ACCATGCTACGGGTCCTTTGCCAAACCTACTAATGTGAAAGGCGGTCAGGCAAAACAAAAAACAACGGAGCAGCCAA ATCAGAAAGTCGAATATGATATCGACATGGAGTTTTTTGATGCGGCATCGCAGAAAACAAACTTAAGTCCTAAAATT GTCATGTATGCAGAAAATGTAAATTTGGAAACTCCAGACACTCATGTAGTGTACAAACCTGGAACAGAAGACACAAG TTCCGAAGCTAATTTGGGACAACAATCTATGCCCAACAGACCCAACTACATTGGCTTCAGAGATAACTTTATTGGAC TTATGTACTATAACAGTACTGGTAACATGGGGGTGCTGGCTGGTCAAGCGTCTCAGTTAAATGCAGTGGTTGACTTG CAGGACAGAAACACAGAACTTTCTTACCAACTCTTGCTTGACTCTCTGGGCGACAGAACCAGATACTTTAGCATGTG GAATCAGGCTGTGGACAGTTATGATCCTGATGTACGTGTTATTGAAAATCATGGTGTGGAAGATGAACTTCCCAACT ACTGTTTTCCACTGGACGGCATAGGTGTTCCAACAACCAGTTACAAATCAATAGTTCCAAATGGAGACAATGCGCCT AATTGGAAGGAACCTGAAGTAAATGGAACAAGTGAGATCGGACAGGGTAATTTGTTTGCCATGGAAATTAACCTTCA AGCCAATCTATGGCGAAGTTTCCTTTATTCCAATGTGGCTCTATATCTCCCAGACTCGTACAAATACACCCCGTCCA ATGTCACTCTTCCAGAAAACAAAAACACCTACGACTACATGAACGGGCGGGTGGTGCCGCCATCTCTAGTAGACACC TATGTGAACATTGGTGCCAGGTGGTCTCTGGATGCCATGGACAATGTCAACCCATTCAACCACCACCGTAACGCTGG CTTGCGTTACCGATCCATGCTTCTGGGTAACGGACGTTATGTGCCTTTCCACATACAAGTGCCTCAAAAATTCTTCG CTGTTAAAAACCTGCTGCTTCTCCCAGGCTCCTACACTTATGAGTGGAACTTTAGGAAGGATGTGAACATGGTTCTA CAGAGTTCCCTCGGTAACGACCTGCGGGTAGATGGCGCCAGCATCAGTTTCACGAGCATCAACCTCTATGCTACTTT TTTCCCCATGGCTCACAACACCGCTTCCACCCTTGAAGCCATGCTGCGGAATGACACCAATGATCAGTCATTCAACG ACTACCTATCTGCAGCTAACATGCTCTACCCCATTCCTGCCAATGCAACCAATATTCCCATTTCCATTCCTTCTCGC AACTGGGCGGCTTTCAGAGGCTGGTCATTTACCAGACTGAAAACCAAAGAAACTCCCTCTTTGGGGTCTGGATTTGA CCCCTACTTTGTCTATTCTGGTTCTATTCCCTACCTGGATGGTACCTTCTACCTGAACCACACTTTTAAGAAGGTTT CCATCATGTTTGACTCTTCAGTGAGCTGGCCTGGAAATGACAGGTTACTATCTCCTAACGAATTTGAAATAAAGCGC ACTGTGGATGGCGAAGGCTACAACGTAGCCCAATGCAACATGACCAAAGACTGGTTCTTGGTACAGATGCTCGCCAA CTACAACATCGGCTATCAGGGCTTCTACATTCCAGAAGGATACAAAGATCGCATGTATTCATTTTTCAGAAACTTCC AGCCCATGAGCAGGCAGGTGGTTGATGAGGTCAATTACAAAGACTTCAAGGCCGTCGCCATACCCTACCAACACAAC AACTCTGGCTTTGTGGGTTACATGGCTCCGACCATGCGCCAAGGTCAACCCTATCCCGCTAACTATCCCTATCCACT CATTGGAACAACTGCCGTAAATAGTGTTACGCAGAAAAAGTTCTTGTGTGACAGAACCATGTGGCGCATACCGTTCT CGAGCAACTTCATGTCTATGGGGGCCCTTACAGACTTGGGACAGAATATGCTCTATGCCAACTCAGCTCATGCTCTG GACATGACCTTTGAGGTGGATCCCATGGATGAGCCCACCCTGCTTTATCTTCTCTTCGAAGTTTTCGACGTGGTCAG AGTGCATCAGCCACACCGCGGCATCATCGAGGCAGTCTACCTGCGTACACCGTTCTCGGCCGGTAACGCTACCACGT AAGAAGCTTCTTGCTTCTTGCAAATAGCAGCTGCAACCATGGCCTGCGGATCCCAAAACGGCTCCAGCGAGCAAGAG CTCAGAGCCATTGTCCAAGACCTGGGTTGCGGACCCTATTTTTTGGGAACCTACGATAAGCGCTTCCCGGGGTTCAT GGCCCCCGATAAGCTCGCCTGTGCCATTGTAAATACGGCCGGACGTGAGACGGGGGGAGAGCACTGGTTGGCTTTCG GTTGGAACCCACGTTCTAACACCTGCTACCTTTTTGATCCTTTTGGATTCTCGGATGATCGTCTCAAACAGATTTAC CAGTTTGAATATGAGGGTCTCCTGCGCCGCAGCGCTCTTGCTACCAAGGACCGCTGTATTACGCTGGAAAAATCTAC CCAGACCGTGCAGGGTCCCCGTTCTGCCGCCTGCGGACTTTTCTGCTGCATGTTCCTTCACGCCTTTGTGCACTGGC CTGACCGTCCCATGGACGGAAACCCCACCATGAAATTGCTAACTGGAGTGCCAAACAACATGCTTCATTCTCCTAAA GTCCAGCCCACCCTGTGTGACAATCAAAAAGCACTCTACCATTTTCTTAATACCCATTCGCCTTATTTTCGCTCCCA TCGTACACACATCGAAAGGGCCACTGCGTTCGACCGTATGGATGTTCAATAATGACTCATGTAAACAACGTGTTCAA TAAACATCACTTTATTTTTTTACATGTATCAAGGCTCTGCATTACTTATTTATTTACAAGTCGAATGGGTTCTGACG AGAATCAGAATGACCCGCAGGCAGTGATACGTTGCGGAACTGATACTTGGGTTGCCACTTGAATTCGGGAATCACCA ACTTGGGAACCGGTATATCGGGCAGGATGTCACTCCACAGCTTTCTGGTCAGCTGCAAAGCTCCAAGCAGGTCAGGA GCCGAAATCTTGAAATCACAATTAGGACCAGTGCTTTGAGCGCGAGAGTTGCGGTACACCGGATTGCAGCACTGAAA CACCATCAGCGACGGATGTCTCACGCTTGCCAGCACGGTGGGATCTGCAATCATGCCCACATCCAGATCTTCAGCAT TGGCAATGCTGAACGGGGTCATCTTGCAGGTCTGCCTACCCATGGCGGGCACCCAATTAGGCTTGTGGTTGCAATCG CAGTGCAGGGGGATCAGTATCATCTTGGCCTGATCCTGTCTGATTCCTGGATACACGGCTCTCATGAAAGCATCATA TTGCTTGAAAGCCTGCTGGGCTTTACTACCCTCGGTATAAAACATCCCGCAGGACCTGCTCGAAAACTGGTTAGCTG CACAGCCGGCATCATTCACACAGCAGCGGGCGTCATTGTTAGCTATTTGCACCACACTTCTGCCCCAGCGGTTTTGG GTGATTTTGGTTCGCTCGGGATTCTCCTTTAAGGCTCGTTGTCCGTTCTCGCTGGCCACATCCATCTCGATAATCTG CTCCTTCTGAATCATAATATTGCCATGCAGGCACTTCAGCTTGCCCTCATAATCATTGCAGCCATGAGGCCACAACG CACAGCCTGTACATTCCCAATTATGGTGGGCGATCTGAGAAAAAGAATGTATCATTCCCTGCAGAAATCTTCCCATC ATCGTGCTCAGTGTCTTGTGACTAGTGAAAGTTAACTGGATGCCTCGGTGCTCCTCGTTTACGTACTGGTGACAGAT GCGCTTGTATTGTTCGTGTTGCTCAGGCATTAGTTTAAAAGAGGTTCTAAGTTCGTTATCCAGCCTGTACTTCTCCA TCAGCAGACACATCACTTCCATGCCTTTCTCCCAAGCAGACACCAGGGGCAAGCTAATCGGATTCTTAACAGTGCAG GCAGCAGCTCCTTTAGCCAGAGGGTCATCTTTAGCGATCTTCTCAATGCTTCTTTTGCCATCCTTCTCAACGATGCG CACGGGCGGGTAGCTGAAACCCACTGCTACAAGTTGCGCCTCTTCTCTTTCTTCTTCGCTGTCTTGACTGATGTCTT GCATGGGGATATGTTTGGTCTTCCTTGGCTTCTTTTTGGGGGGTATCGGAGGAGGAGGACTGTCGCTCCGTTCCGGA GACAGGGAGGATTGTGACGTTTCGCTCACCATTACCAACTGACTGTCGGTAGAAGAACCTGACCCCACACGGCGACA GGTGTTTCTCTTCGGGGGCAGAGGTGGAGGCGATTGCGAAGGGCTGCGGTCCGACCTGGAAGGCGGATGACTGGCAG AACCCCTTCCGCGTTCGGGGGTGTGCTCCCTGTGGCGGTCGCTTAACTGATTTCCTTCGCGGCTGGCCATTGTGTTC TCCTAGGCAGAGAAACAACAGACATGGAAACTCAGCCATTGCTGTCAACATCGCCACGAGTGCCATCACATCTCGTC CTCAGCGACGAGGAAAAGGAGCAGAGCTTAAGCATTCCACCGCCCAGTCCTGCCACCACCTCTACCCTAGAAGATAA GGAGGTCGACGCATCTCATGACATGCAGAATAAAAAAGCGAAAGAGTCTGAGACAGACATCGAGCAAGACCCGGGCT ATGTGACACCGGTGGAACACGAGGAAGAGTTGAAACGCTTTCTAGAGAGAGAGGATGAAAACTGCCCAAAACAACGA GCAGATAACTATCACCAAGATGCTGGAAATAGGGATCAGAACACCGACTACCTCATAGGGCTTGACGGGGAAGACGC GCTCCTTAAACATCTAGCAAGACAGTCGCTCATAGTCAAGGATGCATTATTGGACAGAACTGAAGTGCCCATCAGTG TGGAAGAGCTCAGCCGCGCCTACGAGCTTAACCTCTTTTCACCTCGTACTCCCCCCAAACGTCAGCCAAACGGCACC TGCGAGCCAAATCCTCGCTTAAACTTTTATCCAGCTTTTGCTGTGCCAGAAGTACTGGCTACCTATCACATCTTTTT TAAAAATCAAAAAATTCCAGTCTCCTGCCGCGCTAATCGCACCCGCGCCGATGCCCTACTCAATCTGGGACCTGGTT CACGCTTACCTGATATAGCTTCCTTGGAAGAGGTTCCAAAGATCTTCGAGGGTCTGGGCAATAATGAGACTCGGGCC GCAAATGCTCTGCAAAAGGGAGAAAATGGCATGGATGAGCATCACAGCGTTCTGGTGGAATTGGAAGGCGATAATGC CAGACTCGCAGTACTCAAGCGAAGCATCGAGGTCACACACTTCGCATATCCCGCTGTCAACCTGCCCCCTAAAGTCA TGACGGCGGTCATGGACCAGTTACTCATTAAGCGCGCAAGTCCCCTTTCAGAAGACATGCATGACCCAGATGCCTGT GATGAGGGTAAACCAGTGGTCAGTGATGAGCAGCTAACCCGATGGCTGGGCACCGACTCTCCCAGGGATTTGGAAGA GCGTCGCAAGCTTATGATGGCCGTGGTGCTGGTTACCGTAGAACTAGAGTGTCTCCGACGTTTCTTTACCGATTCAG AAACCTTGCGCAAACTCGAAGAGAATCTGCACTACACTTTTAGACACGGCTTTGTGCGGCAGGCATGCAAGATATCT AACGTGGAACTCACCAACCTGGTTTCCTACATGGGTATTCTGCATGAGAATCGCCTAGGACAAAGCGTGCTGCACAG CACCCTGAAGGGGGAAGCCCGCCGTGATTACATCCGCGATTGTGTCTATCTGTACCTGTGCCACACGTGGCAAACCG GCATGGGTGTATGGCAGCAATGTTTAGAAGAACAGAACTTGAAAGAGCTTGACAAGCTCTTACAGAAATCTCTTAAG GTTCTGTGGACAGGGTTCGACGAGCGCACCGTCGCTTCCGACCTGGCAGACCTCATCTTCCCAGAGCGTCTCAGGGT TACTTTGCGAAACGGATTGCCTGACTTTATGAGCCAGAGCATGCTTAACAATTTTCGCTCTTTCATCCTGGAACGCT CCGGTATCCTGCCCGCCACCTGCTGCGCACTGCCCTCCGACTTTGTGCCTCTCACCTACCGCGAGTGCCCCCCGCCG CTATGGAGTCACTGCTACCTGTTCCGTCTGGCCAACTATCTCTCCTACCACTCGGATGTGATCGAGGATGTGAGCGG AGACGGCTTGCTGGAGTGTCACTGCCGCTGCAATCTGTGCACGCCCCACCGGTCCCTAGCTTGCAACCCCCAGTTGA TGAGCGAAACCCAGATAATAGGCACCTTTGAATTGCAAGGCCCCAGCAGCCAAGGCGATGGGTCTTCTCCTGGGCAA AGTTTAAAACTGACCCCGGGACTGTGGACCTCCGCCTACTTGCGCAAGTTTGCTCCGGAAGATTACCACCCCTATGA AATCAAGTTCTATGAGGACCAATCACAGCCTCCAAAGGCCGAACTTTCGGCCTGCGTCATCACCCAGGGGGCAATTC TGGCCCAATTGCAAGCCATCCAAAAATCCCGCCAAGAATTTCTACTGAAAAAGGGTAAGGGGGTCTACCTTGACCCC CAGACCGGCGAGGAACTCAACACAAGGTTCCCTCAGGATGTCCCAACGACGAGAAAACAAGAAGTTGAAGGTGCAGC CGCCGCCCCCAGAAGATATGGAGGAAGATTGGGACAGTCAGGCAGAGGAGGCGGAGGAGGACAGTCTGGAGGACAGT CTGGAGGAAGACAGTTTGGAGGAGGAAAACGAGGAGGCAGAGGAGGTGGAAGAAGTAACCGCCGACAAACAGTTATC CTCGGCTGCGGAGACAAGCAACAGCGCTACCATCTCCGCTCCGAGTCGAGGAACCCGGCGGCGTCCCAGCAGTAGAT GGGACGAGACCGGACGCTTCCCGAACCCAACCAGCGCTTCCAAGACCGGTAAGAAGGATCGGCAGGGATACAAGTCC TGGCGGGGGCATAAGAATGCCATCATCTCCTGCTTGCATGAGTGCGGGGGCAACATATCCTTCACGCGGCGCTACTT GCTATTCCACCATGGGGTGAACTTTCCGCGCAATGTTTTGCATTACTACCGTCACCTCCACAGCCCCTACTATAGCC AGCAAATCCCGGCAGTCTCGACAGATAAAGACAGCGGCGGCGACCTCCAACAGAAAACCAGCAGCGGCAGTTAGAAA ATACACAACAAGTGCAGCAACAGGAGGATTAAAGATTACAGCCAACGAGCCAGCGCAAACCCGAGAGTTAAGAAATC GGATCTTTCCAACCCTGTATGCCATCTTCCAGCAGAGTCGGGGTCAAGAGCAGGAACTGAAAATAAAAAACCGATCT CTGCGTTCGCTCACCAGAAGTTGTTTGTATCACAAGAGCGAAGATCAACTTCAGCGCACTCTCGAGGACGCCGAGGC TCTCTTCAACAAGTACTGCGCGCTGACTCTTAAAGAGTAGGCAGCGACCGCGCTTATTCAAAAAAGGCGGGAATTAC ATCATCCTCGACATGAGTAAAGAAATTCCCACGCCTTACATGTGGAGTTATCAACCCCAAATGGGATTGGCGGCAGG CGCCTCCCAGGACTACTCCACCCGCATGAATTGGCTCAGCGCCGGGCCTTCTATGATTTCTCGAGTTAATGATATAC GCGCCTACCGAAACCAAATACTTTTGGAACAGTCAGCTCTTACCACCACGCCCCGCCAACACCTTAATCCCAGAAAT TGGCCCGCCGCCCTAGTGTACCAGGAAAGTCCCGCTCCCACCACTGTATTACTTCCTCGAGACGCCCAGGCCGAAGT CCAAATGACTAATGCAGGTGCGCAGTTAGCTGGCGGCTCCACCCTATGTCGTCACAGGCCTCGGCATAATATAAAAC GCCTGATGATCAGAGGCCGAGGTATCCAGCTCAACGACGAGTCGGTGAGCTCTCCGCTTGGTCTACGACCAGACGGA ATCTTTCAGATTGCCGGCTGCGGGAGATCTTCCTTCACCCCTCGTCAGGCTGTTCTGACTTTGGAAAGTTCGTCTTC GCAACCCCGCTCGGGCGGAATCGGGACCGTTCAATTTGTGGAGGAGTTTACTCCCTCTGTCTACTTCAACCCCTTCT CCGGATCTCCTGGGCATTACCCGGACGAGTTCATACCGAACTTCGACGCGATTAGCGAGTCAGTGGACGGCTACGAT TGATGTCTGGTGACGCGGCTGAGCTATCTCGGCTGCGACATCTAGACCACTGCCGCCGCTTTCGCTGCTTTGCCCGG GAACTCATTGAGTTCATCTACTTCGAACTCCCCAAGGATCACCCTCAAGGTCCGGCCCACGGAGTGCGGATTTCTAT CGAAGGCAAAATAGACTCTCGCCTGCAACGAATTTTCTCCCAGCGGCCCGTGCTGATCGAGCGAGACCAGGGAAACA CCACGGTTTCCATCTACTGCATTTGTAATCACCCCGGATTGCATGAAAGCCTTTGCTGTCTTATGTGTACTGAGTTT AATAAAAACTGAATTAAGACTCTCCTACGGACTGCCGCTTCTTCAACCCGGATTTTACAACCAGAAGAACGAAACTT TTCCTGTCGTCCAGGACTCTGTTAACTTCACCTTTCCTACTCACAAACTAGAAGCTCAACGACTACACCGCTTTTCC AGAAGCATTTTCCCTACTAATACTACTTTCAAAACCGGAGGTGAGCTCCAAGGTCTTCCTACAGAAAACCCTTGGGT GGAAGCGGGCCTTGTAGTGCTAGGAATTCTTGCGGGTGGGCTTGTGATTATTCTTTGCTACCTATACACACCTTGCT TCACTTTCTTAGTGGTGTTGTGGTATTGGTTTAAAAAATGGGGCCCATACTAGTCTTGCTTGTTTTACTTTCGCTTT TGGAACCGGGTTCTGCCAATTACGATCCATGTCTAGACTTCGACCCAGAAAACTGCACACTTACTTTTGCACCCGAC ACAAGCCGCATCTGTGGAGTTCTTATTAAGTGCGGATGGGAATGCAGGTCCGTTGAAATTACACACAATAACAAAAC CTGGAACAATACCTTATCCACCACATGGGAGCCAGGAGTTCCCGAGTGGTACACTGTCTCTGTCCGAGGTCCTGACG GTTCCATCCGCATTAGTAACAACACTTTCATTTTTTCTGAAATGTGCGATCTGGCCATGTTCATGAGCAAACAGTAT TCTCTATGGCCTCCTAGCAAGGACAACATCGTAACGTTCTCCATTGCTTATTGCTTGTGCGCTTGCCTTCTTACTGC TTTACTGTGCGTATGCATACACCTGCTTGTAACCACTCGCATCAAAAACGCCAATAACAAAGAAAAAATGCCTTAAC CTCTTTCTGTTTACAGACATGGCTTCTCTTACATCTCTCATATTTGTCAGCATTGTCACTGCCGCTCATGGACAAAC AGTCGTCTCTATCCCTCTAGGACATAATTACACTCTCATAGGACCCCCAATCACTTCAGAGGTCATCTGGACCAAAC TGGGAAGCGTTGATTACTTTGATATAATCTGCAACAAAACAAAACCAATAATAGTAACTTGCAACATACAAAATCTT ACATTGATTAATGTTAGCAAAGTTTACAGCGGTTACTATTATGGTTATGACAGATACAGTAGTCAATATAGAAATTA CTTGGTTCGTGTTACCCAGTTGAAAACCACGAAAATGCCAAATATGGCAAAGATTCGATCCGATGACAATTCTCTAG AAACTTTTACATCTCCCACCACACCCGACGAAAAAAACATCCCAGATTCAATGATTGCAATTGTTGCAGCGGTGGCA GTGGTGATGGCACTAATAATAATATGCATGCTTTTATATGCTTGTCGCTACAAAAAGTTTCATCCTAAAAAACAAGA TCTCCTACTAAGGCTTAACATTTAATTTCTTTTTATACAGCCATGGTTTCCACTACCACATTCCTTATGCTTACTAG TCTCGCAACTCTGACTTCTGCTCGCTCACACCTCACTGTAACTATAGGCTCAAACTGCACACTAAAAGGACCTCAAG GTGGTCATGTCTTTTGGTGGAGAATATATGACAATGGATGGTTTACAAAACCATGTGACCAACCTGGTAGATTTTTC TGCAACGGCAGAGACCTAACCATTATCAACGTGACAGCAAATGACAAAGGCTTCTATTATGGAACCGACTATAAAAG TAGTTTAGATTATAACATTATTGTACTGCCATCTACCACTCCAGCACCCCGCACAACTACTTTCTCTAGCAGCAGTG TCGCTAACAATACAATTTCCAATCCAACCTTTGCCGCGCTTTTAAAACGCACTGTGAATAATTCTACAACTTCACAT ACAACAATTTCCACTTCAACAATCAGCATTATCGCTGCAGTGACAATTGGAATATCTATTCTTGTTTTTACCATAAC CTACTACGCCTGCTGCTATAGAAAAGACAAACATAAAGGTGATCCATTACTTAGATTTGATATTTAATTTGTTCTTT TTTTTTTTATTTACAGTATGGTGAACACCAATCATGGTACCTAGAAATTTCTTCTTCACCATACTCATTTGTGCATT TAATGTTTGCGCTACTTTCACAGCAGTAGCCACAGCAACCCCAGACTGTATAGGAGCATTTGCTTCCTATGCACTTT TTGCTTTTGTTACTTGCATCTGCGTATGTAGCATAGTCTGCCTGGTTATTAATTTTTTCCAACTTATAGACTGGATC CTTGTGCGAATTGCCTACCTGCGCCACCATCCCGAATACCGCAACCAAAATATCGCGGCACTTCTTAGACTCATCTA AAACCATGCAGGCTATACTACCAATATTTTTGCTTCTATTGCTTCCCTACGCTGTCTCAACCCCAGCTGCCTATAGT ACTCCACCAGAACACCTTAGAAAATGCAAATTCCAACAACCGTGGTCATTTCTTGCTTGCTATCGAGAAAAATCAGA AATTCCCCCAAATTTAATAATGATTGCTGGAATAATTAATATAATCTGTTGCACCATAATTTCATTTTTGATATACC CCCTATTTGATTTTGGCTGGAATGCTCCCAATGCACATGATCATCCACAAGACCCAGAGGAACACATTCCCCTACAA AACATGCAACATCCAATAGCGCTAATAGATTACGAAAGTGAACCACAACCCCCACTACTCCCTGCTATTAGTTACTT CAACCTAACCGGCGGAGATGACTGAAACACTCACCACCTCCAATTCCGCCGAGGATCTGCTCGATATGGACGGCCGC GTCTCAGAACAGCGACTCGCCCAACTACGCATCCGCCAGCAGCAGGAACGCGCGGCCAAAGAGCTCAGAGATGTCAT CCAAATTCACCAATGCAAAAAAGGCATATTCTGTTTGGTAAAACAAGCCAAGATATCCTACGAGATCACCGCTACTG ACCATCGCCTCTCTTACGAACTTGGCCCCCAACGACAAAAATTTACCTGCATGGTGGGAATCAACCCCATAGTTATC ACCCAGCAAAGTGGAGATACTAAGGGTTGCATTCACTGCTCCTGCGATTCCATCGAGTGCACCTACACCCTGCTGAA GACCCTATACGGCCTAAGAGACCTGCTACCAATGAATTAAAAAATGATTAATAAAAAATCACTTACTTGAAATCAGC AATAAGGTCTCTGTTGAAATTTTCTCCCAGCAGCACCTCACTTCCCTCTTCCCAACTCTGGTATTCTAAACCCCGTT CAGCGGCATACTTTCTCCATACTTTAAAGGGGATGTCAAATTTTAGCTCCTCTCCTGTACCCACAATCTTCATGTCT TTCTTCCCAGATGACCAAGAGAGTCCGGCTCAGTGACTCCTTCAACCCTGTCTACCCCTATGAAGATGAAAGCACCT CCCAACACCCCTTTATAAACCCAGGGTTTATTTCCCCAAATGGCTTCACACAAAGCCCAAACGGAGTTCTTACTTTA AAATGTTTAACCCCACTAACAACCACAGGCGGATCTCTACAGCTAAAAGTGGGAGGGGGACTTACAGTGGATGACAC CAACGGTTTTTTGAAAGAAAACATAAGTGCCACCACACCACTCGTTAAGACTGGTCACTCTATAGGTTTACCACTAG GAGCCGGATTGGGAACGAATGAAAATAAACTTTGTATCAAATTAGGACAAGGACTTACATTCAATTCAAACAACATT TGCATTGATGACAATATTAACACCTTATGGACAGGAGTCAACCCCACCGAAGCCAACTGTCAAATCATGAACTCCAG TGAATCTAATGATTGCAAATTAATTCTAACACTAGTTAAAACTGGAGCACTAGTCACTGCATTTGTTTATGTTATAG GAGTATCTAACAATTTTAATATGCTAACTACACACAGAAATATAAATTTTACTGCAGAGCTGTTTTTCGATTCTACT GGTAATTTACTAACTAGACTCTCATCCCTCAAAACTCCACTTAATCATAAATCAGGACAAAACATGGCTACTGGTGC CATTACTAATGCTAAAGGTTTCATGCCCAGCACGACTGCCTATCCTTTCAATGATAATTCTAGAGAAAAAGAAAACT ACATTTACGGAACTTGTTACTACACAGCTAGTGATCGCACTGCTTTTCCCATTGACATATCTGTCATGCTTAACCGA AGAGCAATAAATGACGAGACATCATATTGTATTCGTATAACTTGGTCCTGGAACACAGGAGATGCCCCAGAGGTGCA AACCTCTGCTACAACCCTAGTCACCTCCCCATTTACCTTTTACTACATCAGAGAAGACGACTGACAAATAAAGTTTA ACTTGTTTATTTGAAAATCAATTCACAAAATCCGAGTAGTTATTTTGCCTCCCCCTTCCCATTTAACAGAATACACC AATCTCTCCCCACGCACAGCTTTAAACATTTGGATACCATTAGATATAGACATGGTTTTAGATTCCACATTCCAAAC AGTTTCAGAGCGAGCCAATCTGGGGTCAGTGATAGATAAAAATCCATCGGGATAGTCTTTTAAAGCGCTTTCACAGT CCAACTGCTGCGGATGCGACTCCGGAGTCTGGATCACGGTCATCTGGAAGAAGAACGATGGGAATCATAATCCGAAA ACGGTATCGGACGATTGTGTCTCATCAAACCCACAAGCAGCCGCTGTCTGCGTCGCTCCGTGCGACTGCTGTTTATG GGATCAGGGTCCACAGTGTCCTGAAGCATGATTTTAATAGCCCTTAACATCAACTTTCTGGTGCGATGCGCGCAGCA ACGCATTCTGATTTCACTCAAATCTTTGCAGTAGGTACAACACATTATTACAATATTGTTTAATAAACCATAATTAA AAGCGCTCCAGCCAAAACTCATATCTGATATAATCGCCCCTGCATGACCATCATACCAAAGTTTAATATAAATTAAA TGACGTTCCCTCAAAAACACACTACCCACATACATGATCTCTTTTGGCATGTGCATATTAACAATCTGTCTGTACCA TGGACAACGTTGGTTAATCATGCAACCCAATATAACCTTCCGGAACCACACTGCCAACACCGCTCCCCCAGCCATGC ATTGAAGTGAACCCTGCTGATTACAATGACAATGAAGAACCCAATTCTCTCGACCGTGAATCACTTGAGAATGAAAA ATATCTATAGTGGCACAACATAGACATAAATGCATGCATCTTCTCATAATTTTTAACTCCTCAGGATTTAGAAACAT ATCCCAGGGAATAGGAAGCTCTTGCAGAACAGTAAAGCTGGCAGAACAAGGAAGACCACGAACACAACTTACACTAT GCATAGTCATAGTATCACAATCTGGCAACAGCGGGTGGTCTTCAGTCATAGAAGCTCGGGTTTCATTTTCCTCACAA CGTGGTAACTGGGCTCTGGTGTAAGGGTGATGTCTGGCGCATGATGTCGAGCGTGCGCGCAACCTTGTCATAATGGA GTTGCTTCCTGACATTCTCGTATTTTGTATAGCAAAACGCGGCCCTGGCAGAACACACTCTTCTTCGCCTTCTATCC TGCCGCTTAGCGTGTTCCGTGTGATAGTTCAAGTACAACCACACTCTTAAGTTGGTCAAAAGAATGCTGGCTTCAGT TGTAATCAAAACTCCATCGCATCTAATCGTTCTGAGGAAATCATCCACGGTAGCATATGCAAATCCCAACCAAGCAA TGCAACTGGATTGTGTTTCAAGCAGGAGAGGAGAGGGAAGAGACGGAAGAACCATGTTAATTTTTATTCCAAACGAT CTCGCAGTACTTCAAATTGTAGATCGCGCAGATGGCATCTCTCGCCCCCACTGTGTTGGTGAAAAAGCACAGCTAGA TCAAAAGAAATGCGATTTTCAAGGTGCTCAACGGTGGCTTCCAGCAAAGCCTCCACGCGCACATCCAAGAACAAAAG AATACCAAAAGAAGGAGCATTTTCTAACTCCTCAATCATCATATTACATTCCTGCACCATTCCCAGATAATTTTCAG CTTTCCAGCCTTGAATTATTCGTGTCAGTTCTTGTGGTAAATCCAATCCACACATTACAAACAGGTCCCGGAGGGCG CCCTCCACCACCATTCTTAAACACACCCTCATAATGACAAAATATCTTGCTCCTGTGTCACCTGTAGCGAATTGAGA ATGGCAACATCAATTGACATGCCCTTGGCTCTAAGTTCTTCTTTAAGTTCTAGTTGTAAAAACTCTCTCATATTATC ACCAAACTGCTTAGCCAGAAGCCCCCCGGGAACAAGAGCAGGGGACGCTACAGTGCAGTACAAGCGCAGACCTCCCC AATTGGCTCCAGCAAAAACAAGATTGGAATAAGCATATTGGGAACCGCCAGTAATATCATCGAAGTTGCTGGAAATA TAATAAGGCAGAGTTTCTTGTAAAAATTGAATAAAAGAAAAATTTGCCAAAAAAACATTCAAAACCTCTGGGATGCA AATGCAATAGGTTACCGCGCTGCGCTCCAACATTGTTAGTTTTGAATTAGTCTGCAAAAATAAAAAAAAAAACAAGC GTCATATCATAGTAGCCTGACGAACAGATGGATAAATCAGTCTTTCCATCACAAGACAAGCCACAGGGTCTCCAGCT CGACCCTCGTAAAACCTGTCATCATGATTAAACAACAGCACCGAAAGTTCCTCGCGGTGACCAGCATGAATAATTCT TGATGAAGCATACAATCCAGACATGTTAGCATCAGTTAACGAGAAAAAACAGCCAACATAGCCTTTGGGTATAATTA TGCTTAATCGTAAGTATAGCAAAGCCACCCCTCGCGGATACAAAGTAAAAGGCACAGGAGAATAAAAAATATAATTA TTTCTCTGCTGCTGTTCAGGCAACGTCGCCCCCGGTCCCTCTAAATACACATACAAAGCCTCATCAGCCATGGCTTA CCAGACAAAGTACAGCGGGCACACAAAGCACAAGCTCTAAAGTGACTCTCCAACCTCTCCACAATATATATATACAC AAGCCCTAAACTGACGTAATGGGAGTAAAGTGTAAAAAATCCCGCCAAACCCAACACACACCCCGAAACTGCGTCAC CAGGGAAAAGTACAGTTTCACTTCCGCAATCCCAACAGGCGTAACTTCCTCTTTCTCACGGTACGTGATATCCCACT AACTTGCAACGTCATTTTCCCACGGTCGCACCGCCCCTTTTAGCCGTTAACCCCACAGCCAATCACCACACGATCCA CACTTTTTAAAATCACCTCATTTACATATTGGCACCATTCCATCTATAAGGTATATTATTGATGATG

GenBank Accession No. YP_002213828 MTKRVRLSDSFNPVYPYEDESTSQHPFINPGFISPNGFTQSPNGVLTLKCLTPLTTTGGSLQLKVGGGLTVDDTNGF LKENISATTPLVKTGHSIGLPLGAGLGTNENKLCIKLGQGLTENSNNICIDDNINTLWTGVNPTEANCQIMNSSESN DCKLILTLVKTGALVTAFVYVIGVSNNENMLTTHRNINFTAELFFDSTGNLLTRLSSLKTPLNHKSGONMATGAITN AKGFMPSTTAYPFNDNSREKENYIYGTCYYTASDRTAFPIDISVMLNRRAINDETSYCIRITWSWNTGDAPEVQTSA TTLVTSPFTFYYIREDD

GenBank Accession No. YP_002213807 MRRVVLGGAVVYPEGPPPSYESVMQQQQATAVMOSPLEAPFVPPRYLAPTEGRNSIRYSELAPQYDTTRLYLVDNKS ADIASLNYQNDHSNFLTTVVONNDFTPTEASTQTINFDERSRWGGOLKTIMHTNMPNVNEYMESNKFKARVMVSRKP PDGAAVGDTYDHKQDILKYEWFEFTLPEGNFSVTMTIDLMNNAIIDNYLKVGRONGVLESDIGVKFDTRNFKLGWDP ETKLIMPGVYTYEAFHPDIVLLPGCGVDFTESRLSNLLGIRKKOPFQEGFKILYEDLEGGNIPALLDVDAYENSKKE QKAKIEAATAAAEAKANIVASDSTRVANAGEVRGDNFAPTPVPTAESLLADVSEGTDVKLTIQPVEKDSKNRSYNVL EDKINTAYRSWYLSYNYGDPEKGVRSWTLLTTSDVTCGAEQVYWSLPDMMKDPVTFRSTRQVSNYPVVGAELMPVFS KSFYNEQAVYSQQLRQSTSLTHVFNRFPENQILIRPPAPTITTVSENVPALTDHGTLPLRSSIRGVQRVTVTDARRR TCPYVYKALGIVAPRVLSSRTF

GenBank Accession No. YP_002213812 MATPSMLPQWAYMHIAGQDASEYLSPGLVQFARATDTYFNLGNKERNPTVAPTHDVTTDRSQRLMLRFVPVDREDNT YSYKVRYTLAVGDNRVLDMASTFFDIRGVLDRGPSFKPYSGTAYNSLAPKGAPNTSQWIAEGVKNTTGEEHVTEEET NTTTYTEGNAPVKAEAEITKEGLPVGLEVSDEESKPIYADKTYQPEPQLGDETWTDLDGKTEKYGGRALKPDTKMKP CYGSFAKPTNVKGGQAKOKTTEQPNQKVEYDIDMEFFDAASQKTNLSPKIVMYAENVNLETPDTHVVYKPGTEDTSS EANLGQQSMPNRPNYIGFRDNFIGLMYYNSTGNMGVLAGQASQLNAVVDLQDRNTELSYQLLLDSLGDRTRYFSMWN QAVDSYDPDVRVIENHGVEDELPNYCFPLDGIGVPTTSYKSIVPNGDNAPNWKEPEVNGTSEIGOGNLFAMEINLQA NLWRSFLYSNVALYLPDSYKYTPSNVTLPENKNTYDYMNGRVVPPSLVDTYVNIGARWSLDAMDNVNPFNHHRNAGL RYRSMLLGNGRYVPFHIQVPQKFFAVKNLLLLPGSYTYEWNFRKDVNMVLQSSLGNDLRVDGASISFTSINLYATFF PMAHNTASTLEAMLRNDTNDQSFNDYLSAANMLYPIPANATNIPISIPSRNWAAFRGWSFTRLKTKETPSLGSGFDP YFVYSGSIPYLDGTFYLNHTFKKVSIMFDSSVSWPGNDRLLSPNEFEIKRTVDGEGYNVAQCNMTKDWFLVOMLANY NIGYQGFYIPEGYKDRMYSFFRNFQPMSRQVVDEVNYKDFKAVAIPYQHNNSGFVGYMAPTMRQGQPYPANYPYPLI GTTAVNSVTQKKFLCDRTMWRIPFSSNFMSMGALTDLGQNMLYANSAHALDMTFEVDPMDEPTLLYLLFEVFDVVRV HQPHRGIIEAVYLRTPFSAGNATT

GenBank Accession No. AY803294 (SEQ ID NO: 212) CATCATCAATAATATACCTTATAGATGGAATGGTGCCAATATGTAAATGAGGTGATTTTAAAAAGTGTGGGCTGTGT GGTAATTGGCTGTGGGGTTAACGGCTAAAAGGGGCGGCGCGGCCGTGGGAAAATGACGTTTTTTGGGGGTGGAGTGT TTTTGCAAGTTGTCGCGGTAAATGTGACGCAAACAAAGGCTTTTTTTTTACGGAACTACTTAGTGTTCCCACGGTAT TTAACAGGAAATGAGGTAGTTTTGGCCGGATGCAAGTAAAAATTGTTCATTTTCGCGCGAAAACTGAATGAGGAAGT GGTTTTCTGAATAATGCGGTATTTATGGCAGGGTGGAGTATTTGTTCAGGGCCAGGTAGACTTTGACCCATTACGTG GAGGTTTCGATTACCGCGGAGGTTTCGATTACCGTGTTTTTTACCTAAATTTCCGCGTACCGTGTGAAAGTCTTCTG TTTTTACGTAGGTGTCAGCTGATCGCTACGGTATTTATACCTCAGGGTTTGTGTCAAGAGGCCACTCTTGAGTGCCA GCGAGAAGAGTTTTCTCCTCTGCGCCGGCAGTTTAATATTAAAAAAAATGAGACACTTGCGATTTATGCCTCAGGAA ATAATTTCTGCTGAGACTGGAAACGAAATACTGGAGTTTGTGGTGCACGCCCTGATGGGAGACGATCCGGAGCCACC TGTGCAGCTTTTTGAGCCTCCTACGCTTCAGGAACTGTATGATTTAGAGGTAGAGGGATCGGAGGATTCTAATGAGG AAGCTGTGAATGGCTTTTTTACCGATTCTATGCTTTTAGCTGCTAATGAAGGATTAGAATTAGATCCGCCTTTGGAC ACTTTCGATACTCCAGGGGTGATTGTGGAAAGCGGTACAGCTGTAAGAAAATTACCTGATTTGGGTTCCGTGGACTG TGATTTGCACTGCTATGAAGACGGGTTTCCTTTGAGTGATGAGGAGGACCATGAAAAGGAGCAGTCTATGCAGACTG CAGCGGGTGAGGGAGTGAAGGCTGCCATTGGTTTTCAGTTGGATTGCCCGGAGCTTCCTGGACATGGCTGTAAGTCT TGTGAATTTCACAGGAAAAATACTGGAGTAAAGGAACTGTTATGTTCGCTTTGTTATATGAGAGCGCACTGCCACTT TATTTACAGTAAGTGTGTTTAAGTTAAAATTTAAAGGAATATGCTGTTTTTCACATGTATATTGAGTGGGAAATTTG TGCTTCTTATTATAGGTCCTGTGTCTGATGCTGATGAGTCACCATCTCCTGATTCTACTACCTCACCTCCTGAGATT CAAGCACCTGTTCCTGTGGACGTGCACAAGCCCATTCCTGTAAAGCTTAAGCCTGGAAAACGTCCAGCAGTGGAAAA ACTCGAGGACTTGTTACAGGGTGGGGACGGACCTTTGGACTTGAGTACACGGAAACGGCCAAGACAATAAGTGTTCC ATATCCGTGTTTACTTAAGGTGACGTCAATATTTGTGTGAGAGTGCAATGTAATAAAAATATGTTAACTGTGTACTG GTTTTTATTGCTTTTTGGGGGGGGACTCAGGTATATAAGTAGAAGCAGACCTGTGTGGTTAGCTCATAGAAGCTGGC TTTGATTCATGGAGGTTTGGGCCATTTTGGAAGACCTTAGAAAGACTAGGCAACTGTTAGAGAACGCTTCGGACGGA GTCTCCGGTTTTTGGAGATTCTGGTTCGCTAGTGAATTAGCTAGGGTAGTTTTTAGGATAAAACAGGACTATAAAGA AGAATTTGAAAAGTTGTTGGTAGATTGTCCAGGACTTTTTGAAGCTCTTAATTTGGGCCATCAAGTTCACTTTAAAG AAAAAGTTTTATCAGTTTTAGACTTTTCGACCCCAGGTAGAACTGCCGCTGCTGTGGCTTTTCTTACTTTTATATTA GATAAATGGATCCCGCAGACTCATTTCAGCAGGGGATACGTTTTGGATTTCGTAGCCACAGCATTGTGGAGAACATG GAAGGTTCGCAAGATGAGGACAATCTTAGGTTACTGGCCAGTGCAGCCTTTGGGTGTAGCGGGAATCCTGAGGCATC CACCGGTCATGCCAGCGGTTCTGGAGGAGGAACAGCAAGAGGACAACCCGAGAGCCGGCCTGGACCCTCCAGTGGAG GAGGCGGAGTAGCTGACTTGTCTCCTGAACTGCAACGGGTGCTTACTGGATCTACGTCCACTGGACGGGATAGGGGC GTTAAAAGGGAGAGGGCATCTAGTGGTACTGATGCTAGATCTGAGTTGGCTTTAAGTTTAATGAGTCGCAGACGTCC TGAAACCATTTGGTGGCATGAGGTCCAGAAAGAGGGAAGGGATGAAGTTTCTGTATTGCAGGAGAAATATTCACTGG AACAGGTGAAAACATGTTGGTTGGAGCCTGAGGATGATTGGGAGGTGGCCATTAAAAATTATGCCAAGATAGCTTTG AGGCCTGATAAACAGTATAAGATTACTAGACGGATTAATATCCGGAATGCTTGTTACATATCTGGAAATGGGGCTGA GGTGGTAATAGATACTCCAGACAAGACAGTTATTAGATGCTGCATGATGGATATGTGGCCTGGAGTAGTCGGTATGG AAGCAGTAACTTTTGTAAATGTTAAGTTTAGGGGAGATGGTTATAATGGAATAGTGTTTATGGCCAATACCAAACTT ATATTGCATGGTTGTAGCTTTTTTGGTTTTAACAATACCTGTGTAGATGCCTGGGGACAGGTTAGTGTACGGGGATG TAGTTTCTATGCGTGTTGGATTGCCACAGCTGGCAGAACCAAGAGTCAATTGTCTCTGAAGAAATGCATATTCCAAA GATGTAACCTGGGCATTCTTAATGAAGGCGAAGCAAGGGTCCGCCACTGCGCTTCTACAGATACTGGATGTTTTATT TTAATTAAGGGCAATGCCAGCGTAAAGCATAACATGATTTGCGGTGCTTCCGATGAGAGGCCTTATCAAATGCTCAC TTGTGCCGGAGGGCATTGTAACATGCTGGCTACTGTGCATATTGTTTCTCATCAACGCAAAAAATGGCCTGTTTTTG ATCACAATGTGTTGACCAAGTGTACCATGCATGCAGGTGGGCGTAGAGGAATGTTTATGCCTTACCAGTGTAACATG AATCATGTAAAAGTGTTGTTGGAACCAGATGCCTTTTCCAGAATGAGTCTAACAGGAATGTTTGACATGAACATGCA AATCTGGAAGATCCTGAGGTATGATGATACAAGATCGAGGGTGCGCGCATGCGAATGCGGAGGCAAGCATGCCAGGT TCCAGCCGGTGTGTGTAGATGTGACTGAAGATCTGAGACCGGATCATTTGGTTATTGCCCGCACTGGAGCAGAGTTC GGATCCAGTGGAGAAGAAACTGACTAAGGTGAGTATTGGGAAAACTTTGGGGTGGGATTTTCAGATGGACAGATTGA GTAAAAATTTGTTTTTCTGTCTTGCAGCTGTCATGAGTGGAAACGCTTCTTTTAATGGGGGAGTCTTCAGCCCTTAT CTGACAGGGCGTCTCCCATCCTGGGCAGGAGTTCGTCAGAATGTTATGGGATCTACTGTGGATGGAAGACCCGTCCA ACCCGCCAATTCTTCAACGCTGACCTATGCTACTTTAAGTTCTTCACCTTTGGACGCAGCTGCAGCCGCCGCTGCCG CCTCTGTTGCCGCTAACACTGTGCTTGGAATGGGTTACTATGGAAGCATCCTGGCTAATTCCACTTCCTCTAATAAC CCTTCTACCCTGACTCAGGACAAGTTACTTGTCCTTTTGGCCCAGCTGGAGGCTTTGACCCAACGTCTGGGTGAACT TTCTCAGCAGGTGGCCGAGTTGCGAGTACAAACTGAGTCTGCTGTCGGCACGGCAAAGTCTAAATAAAAAAAAAAAA TTCCAGAATCAATGAATAAATAAACGAGCTTGTTGTTGATTTAAAATCAAGTGTTTTTTATTTCATTTTTCGCGCAC GGTATGCCCTAGACCACCGATCTCGATCATTGAGAACACGGTGGATTTTTTCCAAAATCCTATAAAGGTGGGATTGA ATGTTTAGATACATGGGCATTAGGCCGTCTTTGGGGTGGAGATAGCTCCATTGAAGGGATTCATGCTCCGGGGTAGT GTTGTAAATTACCCAGTCATAACAAGGTCGCTGTGCATGGTGTTGCACAATATCTTTTAGAAGTAGGCTGATTGCCA CAGATAAGCCCTTGGTGTAGGTGTTTACAAACCGGTTGAGCTGGGAGGGGTGCATTCGGGGTGAAATTATGTGCATT TTGGATTGGATTTTTAAGTTGGCAATATTGCCGCCAAGATCTCGTCTTGGGTTCATGTTATGAAGTACCACCAAGAC GGTGTATCCGGTACATTTAGGAAATTTATCGTGCAGCTTGGATGGAAAAGCGTGGAAAAATTTGGAGACACCCTTGT GTCCTCCGAGATTTTCCATGCACTCATCCATGATAATAGCAATAGGGCCGTGGGCAGCAGCGCGGGCAAACACGTTC CGTGGGTCTGACACATCATAGTTATGTTCCTGAGTTAAATCATCATAGGCCATTTTAATAAATTTGGGACGGAGAGT ACCCGATTGGGGTATGAATGTTCCTTCGGGCCCCGGAGCATAGTTCCCCTCACAGATTTGCATTTCCCAAGCTTTCA GTTCCGAGGGTGGAATCATGTCCACCTGGGGGGCTATAAAGAACACCGTTTCTGGGGCTGGGGTAATTAGTTGGGAT GATAGCAAGTTTCTGAGCAATTGAGATTTGCCACATCCGGTGGGGCCATAAATGATTCCGATTACAGGTTGCAGTTG GTAGTTTAGGGAACGGCAACTGCCGTCTTCTCGAAGCAAGGGGGCCACCTCGTTCATCATTTCCCTTACATGCATAT TTTCCCGCACCAAATCCATTAGGAGGCGCTCTCCTCCTAGTGATAGAAGTTCTTGTAGTGAGGAAAAGTTTTTCAGC GGTTTTAGACCGTCAGCCATGGGCATTTTGGAGAGAGTCTGTTGCAAAAGTTCTAGTCTGTTCCACAGTTCAGTGAT GTGTTCTATGGCATCTCGATCCAGCAGACCTCCTCGTTTCGCGGGTTTGGACGGCTCCTGGAGTAGGGTATGAGACG ATGGGCGTCCAGCGCTGCCAGGGTTCGGTCCTTCCAGGGTCTCAAAGTTCGGGTCAGGGTTGTTTCCGTCACAGTGA AGGGGTGTGCGCCTGCTTGGGCGCTTGCCAGGGTGCGCTTCAGACTCATCCTGCTGGTCGAAAACTTGTGCCGCTTG GCGCCCTGTATGTCGGCCAAGTAGCAGTTTACCATGAGTTCGTAGTTGAGCGCCTCGGCTGCGTGGCCCTTGGCGCG GAGCTTACCTTTGGAAGTTTTCTTGCATACCGGGCAGTATAGGCATTTCAGCGCATACAGCTTGGGCGCAAGGAAAA TGGATTCTGGGGAGTATGCATCCGCGCCGCAGGAGGCGCAAACAGTTTCACATTCCACCAGCCAGGTTAAATCCGGT TCATTGGGGTCAAAAACAAGTTTTCCGCCATATTTTTTGATGCGTTTCTTACCTTTGGTCTCCATGAGTTCGTGTCC TCGTTGAGTGACAAACAGGCTGTCCGTGTCCCCGTAGACTGATTTTACAGGCCTCTTTTCCAGTGGAGTGCCTCGGT CTTCTTCGTATAGGAACTCTGACCACTCTGATACAAAGGCGCGCGTCCAGGCCAGCACAAAGGAGGCTATGTGGGAG GGGTAGCGATCGTTGTCAACCAGGGGGTCCACCTTTTCCAAAGTATGCAAACACATGTCACTCTCTTCAACATCCAG GAATGTGATTGGCTTGTAGGTGTATTTCACGTGACCTGGGGTCCCAGCTGGGGGGGTATAAAAGGGGGCGGTTCTCT GCTCTTCCTCACTGTCTTCCGGATCGCTGTCCAGGAACGTCAGCTGTTGGGGTAGGTATTCCCTCTCGAAGGGGGC ATGACCTCTGCACTCAGGTTGTCAGTTTCTAAGAACGAGGAGGATTTGATATTGACAGTGCCGCTTGAGATGCCTTT CATGAGGTTTTCGTCCATTTGGTCAGAAAACACAATTTTTTTATTGTCAAGTTTGGTGGCAAATGATCCATACAGGG CGTTGGATAAAAGTTTGGCAATGGATCGCATGGTTTGGTTCTTTTCCTTGTCCGCGCGCTCTTTGGCAGCGATGTTG AGTTGGACATATTCGCGTGCCAGGCACTTCCATTCGGGGAAGATAGTTGTCAATTCATCTGGCACAATTCTCACTTG CCACCCTCGGTTATGCAAGGTAATTAAATCCACACTGGTGGCCACCTCGCCTCGAAGGGGTTCGTTGGTCCAGCAGA GCCTACCTCCTTTCCTAGAACAGAAAGGTGGAAGTGGGTCTAGCATAAGTTCATCGGGAGGGTCTGCATCCATGGTA AAGATTCCAGGAAGTAAATCCTTATCAAAATAGCTGATGGGAGTGGGGTCATCTAAGGCCATTTGCCATTCTCGAGC TGCCAGTGCGCGCTCATATGGGTTAAGGGGACTGCCCCAGGGCATGGGATGGGTGAGTGCAGAGGCATACATGCCAC AGATGTCATAGACGTAGATGGGATCCTCAAAGATGCCTATGTAGGTTGGATAGCATCGCCCCCCTCTGATACTTGCT CGCACATAGTCATATAGTTCATGTGACGGCGCTAGCAGCCCCGGACCCAAGTTGGTGCGATTGGGTTTTTCTGTTCT GTAGACAATCTGGCGAAAGATGGCGTGAGAATTGGAAGAGATGGTGGGTCTTTGAAAAATGTTGAAGTGGGCATGAG GTAGACCTACAGAGTCTCTGATAAAGTGGGCATAAGATTCTTCAAGCTTGGTTACCAGTTGGGCGGTGACAAGTACG TCCAGGGCGCAGTAGTCAAGTGTTTCTTGAATGATGTCATAACCTGGTTGGTTTTTCTTTTCCCACAGTTCGCGGTT GAGAAGGTATTCTTCGCGATCCTTCCAGTACTCTTCTAGCGGAAACCCGTCTTTGTCTGCACGGTAAGATCCTAGCA TGTAGAACTGATTAACTGCCTTGTAAGGGCAGCAGCCCTTCTCTACGGGTAGAGAGTATGCTTGAGCAGCTTTTCGT AGCGAAGCGTGAGTAAGGGCGAAGGTGTCTCTAACCATGACTTTGACAAATTGGTATTTAAAGTCCATGTCGTCACA GGCTCCCTGTTCCCAGAGTTGGAAGTCTACCCGTTTCTTGTAGGCGGGGTTGGGCAAAGCGAAAGTAACATCGTTGA AGAGAATCTTACCGGCTCTGGGCATAAAATTGCGAGTGATGCGAAAAGGCTGTGGTACTTCCGCTCGATTGTTGATC ACCTGGGCAGCTAGGACGATCTCGTCGAAGCCGTTGATGTTGTGTCCTACAATGTATAATTCTATGAAACGCGGCGT GCCTCTGACGTGAGGTAGCTTATTGAGCTCATCAAAGGTTAGGTCTGTAGGGTCAGATAAGGCGTAGTGTTCAAGGG CCCATTCGTGCAGATGAGGATTTGCATGTAGGAATGATGACCAAAGATCCACCGCCAGTGCTGTTTGTAACTGGTCC CGATACTGACGAAAATGCTGGCCAATTGCCATTTTTTCTGGAGTGACACAGTAGAAGGTTCCGGGATCTTGTTGCCA TCGATCCCACTTAAGTTTAATGGCTAGATCGTGGGCCATGTTGACGAGACGCTCTTCTCCTGAGAGTTTCATGACCA GCATGAAAGGAACTAGTTGTTTGCCAAAGGACCCCATCCAGGTGTAAGTTTCCACATCGTAGGTCAGGAAGAGTCTT TCTGTGCGAGGATGAGAGCCGATTGGGAAAAACTGGATTTCCTGCCACCAGTTGGAGGATTGGCTGTTGATGTGATG GAAGTAGAAGTTTCTGCGGCGCGCCGAGCATTCGTGTTTGTGCTTGTACAGACGGCCGCAGTAGTCGCAGCGTTGCA CGGGTTGTATCTCGTGAATGAGCTGTACCTGGCTTCCCTTGACGAGAAATTTCAGTGGGAAGCCGAGGCCTGGCGAT TGTATCTCGTGCTCTTCTATATTCGCTGTATTGGCCTGTTCATCTTCTGTTTCGATGGTGGTCATGCTGACGAGCCC CCGCGGGAGGCAAGTCCAGACCTCGGCGCGGGAGGGGCGGAGCTGAAGGACGAGAGCGCGCAGGCTGGAGCTGTCCA GAGTCCTGAGACGCTGCGGACTCAGGTTAGTAGGTAGGGACAGAAGATTAACTTGCATGATCTTTTCCAGGGCGTGC GGGAGGTTTAGATGGTACTTGATTTCCACAGGTTCGTTTGTAGAGACGTCAATGGCTTGCAGGGTTCCGTGTCCTTT GGGTGCCACTACCGTACCTTTGTTTTTTCTTTTGATCGGTGGTGGCTCTCTTGCTTCTTGCATGCTTAAAAGCGGTG ACGGGGACGCGCGCCGGGCGGCAGCGGTTGTTCCGGACCCGGGGGCATGGCTGGTAGTGGCACGTCGGCGCCGCGCA CGGGCAGGTTCTGGTACTGCGCTCTGAGAAGACTTGCGTGCGCCACCACGCGTCGATTGACGTCTTGTATCTGACGT CTTTGGGTGAAAGCTACCGGCCCCGTGAGCTTGAACCTGAAAGAGAGTTCAACAGAATCAATTTCGGTATCGTTAAC GGCAGCTTGTCTCAGTATTTCTTGTACGTCACCAGAGTTGTCCTGGTAGGCGATCTCCGCCATGAACTGCTCGATTT CTTCCTCCTGAAGATCTCCGCGACCCGCTCTCTCGACGGTGGCCGCGAGGTCATTGGAGATACGGCCCATGAGTTGG GAGAATGCATTCATGCCCGCCTCGTTCCAGACGCGGCTGTAAACCACGGCCCCCTCGGAGTCTCTTGCGCGCATCAC CACCTGAGCGAGGTTAAGCTCCACGTGTCTGGTGAAGACCGCATAGTTGCATAGGCGCTGAAAAAGGTAGTTGAGTG TGGTGGCGATGTGTTCGGCGACAAAGAAATACATGATCCATCGTCTCAGCGGCATTTCGCTGACATCGCCCAGAGCT TCCAAGCGCTCCATGGCCTCGTAGAAGTCCACGGCAAAATTAAAAAACTGGGAGTTTCGCGCGGACACGGTCAATTC CTCCTCGAGAAGACGGATGAGTTCGGCTATGGTGGCCCGTACTTCGCGTTCGAAGGCTCCCGGCATCTCTTCTTCCT CTTCTATCTCTTCTTCCACTAACATCTCTTCTTCGTCTTCAGGCGGGGGGGGAGGGGGCACGCGGCGACGTCGACGG CGCACGGGCAAACGGTCGATGAATCGTTCAATGACCTCTCCGCGGCGGCGGCGCATGGTTTCAGTGACGGCGCGGCC GTTCTCGCGCGGTCGCAGAGTAAAAACACCGCCGCGCATCTCCTTAAAGTGGTGACTGGGAGGTTCTCCGTTTGGGA GGGAAAGGGCGCTGATTATACATTTTATTAATTGGCCCGTAGGGACTGCGCGCAGAGATCTAATCGTGTCAAGATCC ACGGGATCTGAAAACCTTTCAACGAAAGCGTCTAACCAGTCACAGTCACAAGGTAGGCTGAGTACGGCTTCTTGTGG GCGGGGGTGGTTATGTGTTCGGTCTGGGTCTTCTATTCCTTCTTCATCTCGGGAAGGTGAGACGATGCTGCTGGTGA TGAAATTAAAGTAGGCAGTTCTAAGACGGCGGATGGTGGCGAGGAGCACCAGGTCTTTGGGTCCGGCTTGCTGGATA CGCAGGCGATTGGCCATTCCCCAAGCATTATCCTGACATCTAGCCAGATCTTTGTAGTAGTCTTGCATGAGCCGTTC TACGGGCACTTCTTCTTCACCCGTTCTGCCATGCATACGTGTGAGTCCAAACCCGCGCATTGGTTGGACCAGTGCCA AGTCAGCTACAACTCTTTCGGCGAGGATGGCTTGCTGTACTTGGGTGAGGGTGGCTTGAAAGTCATCAAAATCCACG AAGCGGTGGTAAGCCCCGGTATTGATGGTGTAAGCACAGTTGGCCATGACTGACCAGTTAACTGTTTGGTGACCATG GCGCACGAGCTCGGTGTATTTAAGGCGCGAATAGGCGCGGGTGTCAAAAATGTAATCGTTGCAGGTGCGCACCAGAT ACTGGTACCCTATAAGAAAATGCGGTGGTGGTTGGCGGTAGAGAGGCCATTGTTCTGTAGCTGGAGCGCCGGGGGCG AGGTCTTCCAACATAAGGCGGTGATAGCCGTAGATGTACCTGGACATCCAGGTGATTCCTGCGGCGGTAGTGGAAGC CCGAGGAAACTCGCGTACGCGGTTCCAAATGTTGCGTAGCGGCATGAAGTAGTTCATTGTAGGCACGGTTTGACCAG TGAGGCGCGCGCAGTCATTGATGCTCTATAGACACGAAGAAAATGAAAGCGTTCAGCGACTCGACTCTGTAGCCTGG AGGAACGTGAACGGGTTGGGTCGCGGTGTACCCCGGTTCAAGACTTGTACTCGAGCCGGCCGGAGCCGCGGCTAACG TGGTATTGGCACTCCCGTCTCGACCCAGCCTACAAAAATCCAGGATACGGAATCGAGTCGTTTTGCTGGTTGCTGAA TGGCAGGGAAGTGAGTCCTATTTTTTTTTTTGCCGCTCAGATGCATCCCGTGCTGCGACAGATGCGTCCCCAACAAC AGCCCCCCTCGCAGCAGCAGCAACCACAAAAGGCTGTCCCTGCAACTACTGCAACTGCCGCCGTGAGCGGTGCGGGA CAGCCCGCCTATGATCTGGACTTGGAAGAGGGCGAAGGACTGGCACGTCTAGGTGCGCCCTCGCCCGAGCGGCATCC GCGAGTTCAACTGAAAAAAGATTCTCGCGAGGCGTATGTGCCCCAACAGAACCTATTTAGAGACAGAAGCGGCGAGG AGCCGGAGGAGATGCGAGCTTCCCGCTTTAACGCGGGTCGTGAGCTGCGTCACGGTTTGGACCGAAGACGAGTGTTG CGGGACGAGGATTTCGAAGTTGATGAAGTGACAGGGATCAGTCCTGCCAGGGCACACGTGGCTGCAGCCAACCTTGT ATCGGCTTACGAGCAGACAGTAAAGGAAGAGCGTAACTTCCAAAAGTCTTTTAATAATCATGTGCGAACCCTGATTG CCCGCGAAGAAGTTACTCTTGGTTTGATGCATTTGTGGGATTTGATGGAAGCTATCATTCAGAACCCTACTAGCAAA CCTCTGACCGCACAGCTGTTTCTGGTGGTGCAACACAGCAGAGACAACGAGGCTTTCAGAGAGGCACTGCTCAACAT CACTGAACCCGAGGGGAGATGGTTGTATGATCTTATCAACATTCTACAGAGTATCATAGTGCAGGAGCGGAGCCTGG GCCTGGCCGAAAAGGTGGCTGCCATCAATTACTCGGTTTTAAGTTTGGGAAAATATTACGCTCGCAAGATCTACAAG ACTCCATACGTTCCCATAGACAAGGAGGTGAAGATAGATGGGTTCTACATGCGTATGACGCTCAAGGTCTTGACCCT GAGCGATGATCTTGGGGTGTACCGCAATGACAGAATGCATCGCGCCGTTAGCGCCAGTAGGAGGCGCGAGTTAAGCG ACAGGGAACTGATGCACAGTTTGCAAAGAGCTCTGACTGGAGCTGGAACAGAGGGTGAGAATTACTTTGACATGGGA GCTGACTTGCAGTGGCAGCCTAGTCGCAGGGCTCTGAGCGCCGCGACGGCAGGATGTGAGCTTCCTTACATAGAAGA GGCGGATGAAGGCGAGGAGGAAGAGGGCGAGTACTTGGAAGACTGATGGCACAACCCGTGTTTTTTGCTAGATGGAA CAGCAAGCACCGGATCCCGCAACGCGGGCGGCGCTGCAGAGCCAGCCGTCCGGCATTAACTCCTCGGACGATTGGAC CCAGGCCATGCAACGTATCATGGCGTTGACGACTCGCAACCCCGAAGCCTTTAGACAGCAACCCCAGGCCAACCGTC TATCGGCCATCATGGAAGCTGTAGTGCCTTCCCGCTCTAATCCCACTCATGAGAAGGTCCTGGCCATTGTAAACGCG TTGGTGGAGAACAAAGCTATTCGTCCAGATGAGGCCGGACTGGTATACAACGCTCTTTTAGAACGCGTGGCTCGCTA CAACAGTAGCAATGTGCAAACCAATTTGGACCGTATGATAACAGATGTACGCGAAGCCGTGTCTCAGCGTGAAAGGT TCCAGCGCGATGCCAACCTTGGTTCGCTGGTGGCGTTAAATGCTTTTTTGAGTACTCAGCCTGCTAATGTGCCGCGT GGTCAACAGGATTATACTAACTTTTTGAGTGCGTTGAGACTGATGGTATCTGAAGTACCTCAGAGCGAAGTGTATCA GTCCGGACCTGACTACTTCTTTCAGACTAGCAGACAGGGTTTGCAGACGGTAAATCTGAGCCAAGCTTTTAAAAACC TTAAAGGTTTGTGGGGAGTGCATGCCCCGGTAGGAGAAAGAGCAACCGTGTCTAGCTTGTTAACTCCAAACTCCCGC CTATTACTACTGTTGGTAGCTCCTTTCACCGACAGCGGCAGCATCGACCGTAATTCCTATTTGGGTTACCTACTAAA CCTGTATCGCGAAGCCATAGGGCAAAGCCAGGTGGACGAGCAGACCTATCAAGAAATTACCCAAGTCAGTCGCGCTT TGGGTCAGGAAGACACTGGCAGTTTGGAAGCCACTCTGAACTTCTTGCTTACCAATCGGTCTCAGAAGATCCCTCCT CAATATGCTCTTACTGCGGAGGAGGAGAGGATCCTTAGATATGTGCAGCAGAGCGTGGGATTGTTTCTGATGCAAGA GGGGGCAACTCCGACTGCGGCATTGGACATGACAGCGCGAAATATGGAGCCCAGCATGTATGCCAGTAACCGGCCTT TCATTAACAAACTGCTGGACTACTTGCACAGAGCTGCCGCTATGAACTCTGATTATTTTACCAATGCCATCCTAAAC CCGCACTGGCTGCCCCCACCTGGTTTCTACACGGGCGAATATGACATGCCCGACCCTAATGACGGGTTTCTGTGGGA CGACGTGGACAGTAATGTTTTTTCACCTCTTTTTGATCATCGCACGTGGAAAAAGGAAGGCGGCGATAGAATGCATT CTTCTGCATCGCTGTCCGGGGTCATGGGTGCTACCGCGGCTGAGCCCGAGTCTGCAAGTCCTTTTCCTAGTCTACCC TTTTCTCTACACAGTGTACGTAGCAGCGAAGTGGGTAGAATAAGTCGCCCGAGTTTAATGGGCGAAGAGGAATACCT AAACGATTCCTTGCTCAGACCGGCGAGAGAAAAAAATTTCCCAAACAATGGAATAGAAAGTTTGGTGGATAAGATGA GTAGATGGAAGACTTATGCTCAGGATCACAGAGACGAGCCTGGGATCATGGGGACTACAAGTAGAGCGAGCCGTAGA CGCCAGCGTCATGACAGACAGAGGGGTCTTGTGTGGGAAGATGAGGATTCGGCCGATGATAGCAGCGTGTTGGACTT GGGTGGGAGAGGAAGGGGCAACCCGTTTGCTCATTTGCGCCCTCGCTTGGGTGGTATGTTGTAAAAAAAAAATAAAA AGGAAAACTCACCAAGGCCATGGCGACGAGCGTACGTTCGTTCTTCTTTATTATTTGTGTCTAGTATAATGAGGCGA GTCGTGCTAGGCGGAGCGGTGGTGTATCCGGAGGGTCCTCCTCCTTCGTACGAGAGCGTGATGCAGCAGCAGCAGGC GACGGCGGTGATGCAATCCCCACTGGAGGCTCCCTTTGTACCTCCGCGATACCTGGCACCTACGGAGGGCAGAAACA GCATTCGTTACTCGGAACTGGCACCTCAGTACGATACCACCAGGTTGTATCTGGTGGACAACAAGTCGGCGGACATT GCTTCTCTGAACTATCAGAATGACCACAGCAACTTCTTGACCACGGTGGTGCAGAACAATGACTTTACCCCTACGGA AGCCAGTACCCAGACCATTAACTTTGATGAACGATCGCGGTGGGGCGGTCAGCTAAAGACCATCATGCATACTAACA TGCCCAACGTAAACGAGTATATGTTTAGTAACAACTTCAAAGCGCGTGTGATGGTGTCCAGAAAACCTCCCGAAGGT GCTGCAGTTGGGGATACATATGATCACAAGCAGGATATTTTGGAATATGAGTGGTTCGAGTTTACTTTGCCAGAAGG CAACTTTTCAGTTACTATGACCATTGATTTGATGAACAATGCCATCATAGATAACTACTTGAAAGTGGGCAGACAGA ATGGAGTGCTTGAAAGTGACATTGGTGTTAAGTTCGACACCAGGAACTTCAAGCTGGGATGGGATCCCGAAACCAAG TTGATTATGCCTGGAGTGTATACGTATGAAGCCTTTCATCCTGACATTGTCTTACTGCCTGGCTGTGGAGTGGACTT TACCGAAAGTCGTTTGAGCAACCTTCTTGGTATCAGAAAAAAACAGCCATTTCAAGAGGGTTTTAAGATTTTGTATG AAGATTTAGAAGGAGGTAATATTCCGGCCCTCTTGGATGTAGATGCCTATGAGAACAGTAAGAAAGAACAAAAAGCC AAAATAGAAGCTGCTGCGGAAGCTAAGGCAAACATAGTTGCCAGCGACTTTACAAGGGTTGCTAACGCTGGAGAGGT CAGAGGAGACAATTTTGCACCAACACCTGTTCCGACTGCAGAATCATTATTGGCCGATGTAACTGGAGGAACGGACG TGAAACTCACTATTCAACCTGTAGAAAAAGATAGTAAGAATAGAAGCTATAATGTGTTGGAAGATAAAATCAACACA GCCTATCGCAGTTGGTACCTTTCGTACAATTATGGCGATCCCGAAAAAGGAGTGCGTTCCTGGACATTGCTCACCAC CTCAGATGTCACCTGCGGAGCAGAGCAGGTCTACTGGTCGCTTCCAGACATGATGCAGGATCCTGTCACTTTCCGCT CCACTAGACAAGTCAGCAACTACCCTGTGGTGGGTGCAGAGCTTATGCCCGTCTTCTCAAAGAGCTTCTACAACGAA CAAGCTGTGTACTCCCAGCAGCTCCGCCAGTCCACCTCGCTTACGCACGTCTTCAACCGCTTTCCTGAGAACCAGAT TTTAATCCGTCCGCCGGCGCCCACCATTACCACCGTCAGTGAAAACGTTCCTGCTCTCACAGATCACGGGACCCTGC CGTTGCGCAGCAGTATCCGGGGAGTCCAACGTGTGACCGTTACTGACGCCAGACGCCGCACCTGTCCCTACGTGTAC AAGGCACTGGGCATAGTCGCACCGCGCGTCCTTTCAAGCCGCACTTTCTAAAAAAATGTCCATTCTTATCTCGCCCA GTAATAACACCGGTTGGGGTCTGCGCGCTCCAAGCAAGATGTACGGAGGCGCACGCAAACGTTCTACCCAACATCCC GTGCGTGTTCGCGGTCATTTTCGCGCTCCATGGGGTGCCCTCAAGGGCCGCACTCGCGTTCGAACCACCGTCGATGA TGTAATCGATGAGGTGGTTGCCGACGCCCGTAATTATACTCCTACTGCGCCTACATCTACTGTGGATGCAGTTATTG ACAGTGTAGTGGCTGACGCTCGCAACTATGCTCGACGTAAGAGCCGGCGAAGGCGCATTGCCAGACGCCACCGAGCT ACCACTGCCATGCGAGCCGCAAGAGCTCTGCTACGAAGAGCTAGACGCGTGGGACGAAGAGCCATGCTTAGGGCGGC CAGACGTGCAGCTTCGGGCGCCAGCGCCGGCAGGTCCCGCAGGCAAGCAGCCGCTGTCGCAGCGGCGACTATTGCCG ACATGGCCCAAACGCGAAGAGGCAATGTATACTGGGTGCGTGACGCTGCCACCGGTCAACGTGTACCCGTGCGCACC CGTCCCCCTCGCACTTAGAAGATACTGAGCAGTCTCCGATGTTGTGTCCCAGCGGCGAGGATGTCCAAGCGCAAATA CAAGGAAGAAATGCTGCAGGTTATCGCACCTGAAGTCTACGGCCAACCGCTGAAGGATGAAAAAAAACCCCGCAAAA TCAAGCGGGCTAAAAAGGACAAAAAAGAAGAGGAAGATGGCGATGATGGGCTGGCGGAGTTTGTGCGCGAGTTTGCC CCACGGCGACGCGTGCAATGGCGTGGACGCAAAGTTCGACATTTGTTGAGACCTGGAACTTCGGTGGTCTTTACACC CGGCGAGCGTTCAAGCGCTACTTTTAAGCGTTCCTATGATGAGGTGTACGGGGATGATGATATTCTTGAGCAGGCGG CTGACCGATTAGGCGAGTTTGCTTATGGCAAGCGTAGTAGAATAAATCCCAAGGATGAGACAGTGTCCATACCCTTG GATCATGGAAATCCCACCCCTAGTCTTAAACCGGTCACTTTGCAGCAAGTGTTACCCGTAACTCCGCGAACAGGTGT TAAACGCGAAGGTGAAGATTTGTATCCCACTATGCAACTAATGGTACCCAAACGCCAAAAGTTGGAGGACGTTTTGG AGAAAGTAAAAGTGGATCCAGATATTCAACCTGAGGTTAAAGTGAGACCCATTAAGCAGGTAGCGCCTGGTCTGGGA GTACAAACTGTAGACATTAAGATTCCCACTGAAAGTATGGAAGTGCAAACTGAACCCGCAAAGCCTACTGCCACCTC CACTGAAGTGCAAACGGATCCATGGATGCCGATGCCTATTACAACTGACGCCGCCGGTCCCACTCGAAGATCCCGAC GAAAGTACGGTCCAGCAAGTCTGTTGATGCCCAACTATGTTGTACACCCATCTATTATTCCTACTCCTGGTTACCGA GGCACTCGCTACTATCGCAGCCGAAACAGTACCTCCCGCCGTCGCCGCAAGACACCTGCAAATCGCAGTCGTCGCCG TAGACGCACAAGCAAACCGACTCCCGGCGCCCTGGTGCGGCAAGTGTACCGCAATAGTAGTGCGGAACCTTTGACAC TGCCGCGTGCGCGTTACCATCCAAGTATCATCACTTAATCAATGTTGCCGCTGCCTCCTTGCAGATATGGCCCTCAC TTGTCGCCTTCGCGTTCCCATCACTGGTTACCGAGGAAGAAATTCGCGCCGTAGAAGAGGGATGTTGGGGCGCGGAA TGCGACGCTACAGGCGACGGCGTGCTATCCGCAAGCAATTGCGGGGTGGTTTTTTGCCAGCCTTAATTCCAATTATC GCTGCTGCAATTGGCGCGATACCAGGCATAGCTTCCGTGGCGGTTCAGGCCTCGCAACGACATTGACATTGGAAAAA AAAGTATAAATAAAAAAAAAAATACAATGGACTCTGACACTCCTGGTCCTGTGACTATGTTTTCTTAGAGATGGAAA ACATCAATTTTTCATCCTTGGCTCCGCGACACGGCACGAAGCCGTACATGGGCACCTGGAGCGACATCGGCACGAGC CAACTGAACGGGGGCGCCTTCAATTGGAGCAGTATCTGGAGCGGGCTTAAAAATTTTGGCTCAACCATAAAAACATA CGGGAACAAAGCTTGGAACAGCAGTACAGGACAGGCGCTTAGAAATAAACTTAAAGACCAGAACTTTCAACAAAAAG TAGTCGATGGGATAGCTTCCGGCATCAATGGAGTGGTAGATTTGGCTAATCAGGCTGTGCAGAAAAAGATAAACAGT CGTTTGGACCCGCCGCCAGCAACCCCAGGTGAAATACAAGTGGAGGAAGAAATTCCTCCGCCAGAAAAACGAGGCGA CAAGCGTCCGCGTCCCGATTTGGAAGAGACGCTGGTGACGCGCGTAGATGAACCGCCTTCTTATGAGGAAGCAACGA AGCTTGGAATGCCCACCACTAGACCGATAGCCCCTATGGCTACCGGGGTAATGAAACCTTCTCAGTTGCATCGACCC GTCACTTTGGATTTGCCCCCTCCCCCTGCTGCTACTGCTGTACCCGCTTCTAAGCCTGTCGCTGCCCCGAAACCAGT CGCCGTAGCCAGGTCACGTCCCGGGGGCGCTCCTCGTCCAAATGCGCACTGGCAAAATACTCTGAACAGCATCGTGG GTCTAGGCGTGCAAAGTGTAAAACGCCGTCGCTGCTTTTAATTAAATATGGAGTAGCGCTTAACTTGCCTATCTGTG TATATGTGTCATTACACGCCGTCACAGCAGCAGAGGAAAAAAGGAAGAGGTCGTGCGTCGACGCTGAGTTACTTTCA AGATGGCCACCCCATCGATGCTGCCCCAGTGGGCATACATGCACATCGCCGGACAGGATGCTTCGGAGTACCTGAGT CCGGGTCTGGTGCAGTTCGCCCGCGCCACAGACACCTACTTCAATCTGGGAAATAAGTTTAGAAATCCCACCGTAGC GCCAACCCACGATGTGACCACCGACCGTAGCCAGCGGCTCATGTTGCGCTTCGTGCCCGTTGACCGGGAGGACAATA CATACTCTTACAAAGTGCGGTACACCCTGGCCGTGGGCGACAACAGAGTGCTGGATATGGCCAGCACGTTCTTTGAC ATTAGGGGCGTGTTGGACAGAGGTCCCAGTTTCAAACCCTATTCTGGTACGGCTTACAACTCTCTGGCTCCTAAAGG CGCTCCAAATGCATCTCAGTGGTTGGATAAAGGGGTTGAAACTACTGAAGAACGGCAAAATGAAGACGGGGAAAATG ACGAAAAAGCTACATACACTTTTGGCAATGCCCCAGTAAAAGCCGATGCTGACATTACAAAAGACGGACTACCAATA GGTTTGGAAGTCCCAGCTGAAGGTGACCCTAAACCTATCTACGCTAATAAGCTTTACCAACCAGAACCCCAGGTGGG ACAGGAATCGTGGACTGATACAGATGGCACTGAAGAAAAATACGGAGGCAGAGTACTTAAACCGGACACTAAAATGA AACCGTGCTATGGGTCTTTTGCTAAACCTACTAATGTGAAAGGCGGACAGGCAAAAGTGAAAACAGAAGAAGGCAAC AACATTGAATATGACATTGACATGAACTTTTTTGACTTAAGATCACAAAAACAAGGTCTTAAACCTAAGATTGTAAT GTATGCAGAAAATGTGGACCTGGAATCTCCAGATACTCATGTTGTGTACAAACCTGAAGTTTCAGATGCTAGTTCAA ATGCTAATCTTGGACAGCAGTCTATGCCCAACAGACCCAACTACATTGGCTTCAGAGATAATTTTATTGGTCTTATG TACTATAACAGTACTGGTAACATGGGGGTGCTGGCTGGCCAAGCATCTCAGTTGAATGCAGTGGTTGACTTGCAGGA CAGAAACACAGAACTGTCTTACCAACTCTTGCTTGACTCCCTGGGCGATAGAACCAGATACTTTAGCATGTGGAATC AGGCTGTTGACAGTTATGATCCCGATGTGCGTGTTATTGAAAATCATGGTGTGGAAGATGAACTTCCCAACTACTGT TTTCCACTGGACGGCATCGGTCCGCGAACAGATAGTTACAAGGAGATTCAGTTAAATGGAGACCAAGCTTGGAAAGA TGTAAATCCAAATGGTATCAGTGAACTTGTTAAGGGAAATCCATTTGCCATGGAAATTAACCTTCAAGCCAATCTAT GGCGAAGTTTCCTTTATTCCAATGTGGCTCTGTATCTCCCAGACTCGTACAAATACACCCCGTCCAATGTCACTCTT CCAGAAAACAAAAACACCTACGACTACATGAACGGGCGGGTGGTGCCGCCATCTCTAGTAGACACCTATGTGAACAT TGGCGCCAGGTGGTCTCTGGATGCTATGGACAATGTCAACCCATTCAACCACCACCGTAACGCTGGCTTGCGTTACC GATCCATGCTTTTGGGTAACGGACGTTATGTGCCTTTCCACATACAAGTGCCTCAAAAATTCTTCGCTGTCAAAAAC CTGCTGCTTCTCCCAGGCTCCTACACTTATGAGTGGAACTTCAGGAAGGATGTGAACATGGTGCTACAGAGTTCCCT CGGTAACGACCTACGGGTAGATGGCGCCAGCATCAGTTTCACGAGCATCAACCTCTATGCTACCTTTTTCCCCATGG CTCACAACACCGCTTCCACCCTTGAAGCCATGCTGCGGAATGACACCAATGATCAGTCATTCAACGACTATCTATCT GCAGCTAACATGCTCTATCCCATTCCTGCCAATGCAACCAATATTCCCATTTCCATTCCTTCTCGCAACTGGGCGGC TTTCAGAGGCTGGTCATTTACCAGACTCAAAACCAAAGAAACTCCCTCTTTGGGGTCTGGATTTGACCCCTACTTTG TCTATTCTGGTTCTATTCCCTACCTGGATGGTACCTTCTACCTGAACCACACTTTTAAGAAGGTTTCCATCATGTTT GACTCTTCAGTGAGCTGGCCTGGAAATGACAGGTTACTATCTCCCAACGAATTTGAAATAAAGCGCACTGTGGATGG CGAAGGCTACAATGTAGCCCAATGCAACATGACCAAAGACTGGTTCTTGGTACAGATGCTCGCCAACTACAACATAG GCTATCAGGGCTTCTACATTCCAGAAGGATACAAAGATCGCATGTATTCATTTTTCAGAAACTTCCAGCCCATGAGC AGGCAGGTGGTTGATGAGGTCAATTACAAAGACTTCAAGGCCGTCGCCATACCCTACCAACACAACAACTCTGGCTT TGTGGGTTACATGGCTCCGACCATGCGCCAAGGTCAACCCTATCCCGCTAACTATCCCTATCCACTCATTGGAACAA CTGCCGTAAATAGTGTTACGCAGAAAAAGTTCTTGTGTGACAGAACCATGTGGCGCATACCGTTCTCGAGCAACTTC ATGTCTATGGGGGCCCTTACAGACTTGGGACAGAACATGCTTTATGCCAACTCAGCTCATGCTCTGGACATGACCTT TGAGGTGGATCCCATGGATGAGCCCACCCTGCTTTATCTTCTCTTCGAAGTTTTCGACGTGGTCAGAGTGCATCAGC CACATCGCGGCATCATCGAGACAGTCTACCTGCGTACACCGTTCTCGGCCGGTAACGCTACCACGTAAAAAGCTTCT TGCTTCTTGCAAACAGCAGCTGCAACCATGGCCTGCGGATCCCAAAACGGCTCCAGCGAGCAAGAGCTCAGAGCCAT TGTCCAAGACCTGGGTTGCGGACCCTATTTTTTGGGAACCTACGATAAGCGCTTCCCGGGGTTCATGGCCCCCGATA AGCTCGCCTGTGCCATTGTAAACACGGCCGGACGTGAGACGGGGGGAGAGCACTGGTTGGCTTTCGGTTGGAACCCA CGTTCTAACACCTGCTACCTTTTTGATCCTTTTGGATTCTCGGATGATCGTCTTAAACAGATTTACCAGTTTGAATA TGAGGGTCTCCTGCGCCGCAGCGCTCTTGCTACCAAGGACCGCTGTATTACGCTGGAAAAATCTACCCAGACCGTGC AGGGCCCCCGTTCTGCCGCCTGCGGACTTTTCTGCTGCATGTTCCTTCATGCCTTTGTGCACTGGCCTGACCGTCCC ATGGACGGAAACCCCACCATGAAATTGCTGACTGGAGTGCCAAACAACATGCTTCATTCTCCTAAAGTCCAGCCCAC CCTGTGTGACAATCAAAAAGCACTCTACCATTTTCTCAATACCCATTCGCCTTATTTTCGCTCCCATCGTACACACA TCGAAAGGGCCACTGCGTTCGACCGTATGGATGTGCAATAATGACTCATGTAAACAACGTGTTGAATAAACAGCACT TTATTTTTTACACGTATCAAGGCTCTGGATTACTTATTTATTTACAAGTCGAATGGGTTCTGACGAGAATCAGAATG ACCCGCGGGCAGTGATACGTTGCGGAACTGATACTTGGGTTGCCACTTGAATTCGGGAATCACCAACTTGGGAACCG GTATATCGGGTAGGATGTCACTCCACAGCTTTCTGGTCAGCTGCAAAGCTCCCAGCAGGTCAGGAGCCGAAATCTTG AAATCACAATTAGGACCAGTGCTCTGAGCGCGAGAGTTGCGGTACACCGGATTGCAGCACTGAAACACCATCAGCGA CGGATGTCTCACGCTTGCCAGCACGGTGGGATCTGCAATCATGCCCACATCCAGATCTTCAGCATTGGCAATGCTGA ACGGGGTCATCTTGCAGGTCTGCCTACCCATGGCGGGCACCCAATTAGGCTTGTGGTTGCAATCGCAGTGCAGGGGG ATTAGTATCATCTTGGCCTGATCCTGTCTGATTCCTGGATACACGGCTCTCATGAAAGCATCATATTGCTTGAAAGC CTGCTGGGCTTTACTACCCTCGGTATAGAACATCCCGCAGGACCTGCTCGAAAACTGGTTAGCTGCGCAGCCGGCAT CATTCACACAGCAGCGGGCGTCATTGTTGGCTATTTGCACCACACTTCTGCCCCAGCGGTTTTGGGTGATTTTGGTT CGCTCGGGATTCTCCTTCAAGGCTCGTTGTCCATTCTCGCTGGCCACATCCATCTCGATAATCTGCTCCTTCTGAAT CATAATAGTGCCATGCAGGCACTTCAGCTTGCCCTCATAATCATTGCAGCCATGAGGCCACAACGCACAGCCTGTAC ATTCCCAATTATGGTGGGCGATCTGAGAAAAAGAATGTATCATTCCCTGCAGAAATCTTCCCATCATCGTGCTCAGT GTCTTGTGACTAGTGAAAGTTAACTGGATGCCTCGGTGCTCCTCGTTTACGTACTGGTGACAGATGCGCTTGTATTG TTCGTGCTGCTCAGGCATTAGTTTAAAAGAGGTTCTAAGTTCGTTATCCAGCCTGTACTTCTCCATCAGTACACACA TCACTTCCATGCCCTTCTCCCAAGCAGACACCAGGGGCAAGCTAATCGGATTCTTAACAGTACAGGCAGCAGCTCCT TTAGCCAGAGGATCATCTTTGGCAATCTTTTCAATGCTTCTTTTGCCATCCTTCTCAACGATGCGCACGGGCGGGTA GCTGAAACCTACTGCTACAAGCTGCGCCTCTTCTCTTTCTTCTTCGCTGTCTTGACTGATGTCTTGCATGGGAACAT GTTTGGTCTTCCTTGGCTTCTTTTTGGGGGGTATCGGGGGAGGAGGACTGTCGCTCCGTTCCGGAGACAGGGAGGAT TGTGAAGTTTCGCTCACCATTACCAACTGACTGTCGGTAGAAGAACCTGACCCCACACGGCGACAGGTGTTTCTCTT CGGGGGCAGAGGTGGAGGCGATTGCGAAGGGCTGCGGTCCGACCTGGAAGGCGGATGACTGGCAGAACCCCTTCCGC GTTCGGGGGTGTGCTCCCTGTGGCGGTCGCTTAACTGATTTCCTTCGCGGCTGGCCATTGTGTTCTCCTAGGCAGAG AAACAACAGACATGGAAACTCAGCCATTGCTGTCAACATCGCCACAAGTGCCATCACATCTCGTCGTCAGCGACGAG GAAAAGGAGCAGAGCTTAACCATTCCACCGCCCAGTCCTGCCACCACCTCTACCCTAGAAGATAAGGAGGTCGACGC ATCTCATGACATGCAGAATAAAAAAGCGAAAGAGTCTGAAACAGACATCGAGCAAGACCCGGGCTATGTGACACCGG TGGAACACGAGGAAGAGTTGAAACGCTTTCTAGAGAGAGAGGATGAAAACTGCCCAAAACAGCAAGCGGATAACTAT CACCAAGATGCTGGAAATAGGGATCAGAACACCGACTACCTCATAGGGCTTGACGGGGAAGACGCGCTCCTTAAACA TCTAGCAAGACAGTCACTCATAGTCAAGGATGCATTATTGGACAGAACTGAAGTGCCCATCAGTGTGGAAGAGCTCA GCCGCGCCTACGAGCTTAACCTTTTTTCACCTCGTACTCCCCCCAAACGCCAGCCAAACGGCACCTGCGAGCCAAAT CCTCGCTTAAACTTTTATCCAGCTTTTGCTGTGCCAGAAGTACTCGCTACTTATCACATCTTTTTTAAAAATCAAAA AATTCCAGTCTCCTGCCGCGCTAATCGCACCCGCGCTGACGCCCTACTTAATCTGGGACCTGGTTCACGCTTACCTG ATATAGCTTCCTTGGAAGAGGTTCCAAAAATCTTCGAGGGTCTGGGCAATAATGAGACTCGGGCCGCAAATGCTCTG CAAAAGGGAGAAAATGGCATGGATGAGCATCACAGCGTTCTGGTGGAATTGGAGGGCGATAATGCCAGACTCGCAGT ACTCAAGCGAAGCGTCGAGGTCACACACTTTGCATACCCCGCTGTCAACCTGCCCCCTAAAGTTATGACGGCGGTCA TGGACCAGTTACTCATTAAGCGCGCAAGTCCCCTTTCAGAAGACATGCATGACCCAGACGCCTGTGATGAGGGTAAA CCAGTGGTCAGTGATGAGCAGCTAACCCGATGGCTGGACACCGACTCTCCCCGGGATTTGGAAGAGCGTCGCAAGCT TATGATGGCCGTAGTGCTGGTTACCGTAGAACTAGAGTGTCTCCGGCGTTTCTTTACCGATTCAGAAACCTTGCGCA AACTCGAAGAGAATCTGCACTACACTTTTAGACACGGCTTTGTGCGGCAGGCGTGCAAGATATCTAACGTGGAACTC ACCAACCTGGTTTCCTACATGGGTATTCTGCATGAGAATCGTCTAGGACAAAGCGTGCTGCACAGCACCCTTAAGGG GGAAGCCCGCCGTGATTACATCCGCGATTGTGTCTATCTCTACCTGTGCCACACGTGGCAAACCGGCATGGGTGTAT GGCAGCAATGTTTAGAAGAACAGAACTTGAAAGAGCTTAACAAGCTCTTACAGAAATCTCTTAAGGTTCTGTGGACA GGGTTCGACGAGCGCACCGTCGCTTCCGACCTGGCAGACCTCATCTTCCCAGAGCGTCTTAGGGTTACTTTGCGAAA CGGACTGCCTGACTTTATGAGCCAGAGCATGCTTAACAATTTTCGCTCTTTCATCCTGGAACGCTCCGGTATCCTGC CCGCCACCTGCTGCGCACTGCCCTCCGACTTTGTGCCTCTCACCTACCGCGAGTGCCCCCCGCCGCTATGGAGTCAC TGCTACCTGTTCCGTCTGGCCAACTACCTCTCCTACCACTCGGATGTGATCGAGGATGTGAGCGGAGACGGCTTGCT GGAGTGTCACTGCCGCTGCAATCTGTGCACGCCCCACCGGTCCCTAGCTTGCAACCCCCAGTTGATGAGCGAAACCC AGATAATAGGCACCTTTGAACTGCAAGGCCCCAGCAGCCAAGGCGATGGGTCTTCTCCTGGGCAAAGTTTAAAACTG ACCCCGGGACTGTGGACCTCTGCCTACTTGCGCAAGTTTGCCCCGGAAGATTACCACCCCTATGAAATCAAGTTCTA TGAGGACCAATCACAGCCTCCAAAGGCCGAACTTTCGGCCTGCGTCATCACCCAGGGGGCAATTCTAGCCCAATTGC AAGCCATCCAAAAATCCCGCCAAGAATTTCTACTGAAAAAGGGTAAGGGGGTCTACCTTGACCCCCAGACCGGCGAG GAACTCAACACAAGGTTCCCTCAGGATGTCCCAACGACGAGAAAGCAAGAAGTTGAAGGTGCAGCCGCCGCCCCCAG AAGATATGGAGGAAGATTGGGACAGTCAGGCAGAGGAAGCGGAGGAGGACAGTCTGGAGGACAGTCTGGAGGAAGAC AGTTTGGAGGAGGAAAACGAGGAGGCAGAGGAGGTGGAAGAAGTAACCGCCGACAAACAGTTATCCCCGGCTGCGGA GACAAGCAACAGCGCTATCATCTCCGCTCCGAGTCGAGGAACGCGGCGGCGTCCCAGCAGTAGATGGGACGAGACCG GACGCTTCCCGAACCCAACCACCGCTTCCAAGACCGGTAAGAAGGATCGGCAGGGATACAAGTCCTGGCGGGGGCAT AAGAATGCCATCATCTCCTGCTTGCATGAGTGCGGGGGAAACATATCCTTCACGCGACGCTACTTGCTATTCCACCA TGGGGTGAACTTTCCACGCAATGTTTTGCATTACTACCGTCACCTCCACAGCCCCTACTATAGCCAGCAAATCCCGG CAATCTCGACAGAAAAAGACAGCGGCGGCGACCTCCAACAGAAAACCAGCAGCGGCAGTTAAAAAATACACAACAAG TGCAGCAACAGGAGGATTAAAGATTACAGCCAACGAGCCAGCGCAAACCCGAGAGCTAAGAAATCGGATCTTTCCAA CCCTGTATGCCATCTTCCAGCAGAGTCGGGGCCAAGAGCAGGAACTGAAAATAAAAAACCGATCTTTGCGTTCGCTC ACCAGAAGTTGTTTGTATCACAAGAGCGAAGATCAACTTCAGCGCACTCTTGAGGACGCCGAGGCTCTCTTCAACAA GTACTGCGCGCTGACTCTTAAAGAGTAGGCAGCGACCGCGCTTATTCAAAAAAGGCGGGAATTACATCATCCTCGTC ATGAGTAAAGAAATTCCCACGCCTTACATGTGGAGTTATCAGCCCCAAATGGGATTGGCGGCAGGCGCCTCCCAGGA CTACTCCACCCGCATGAATTGGCTCAGCGCCGGGCCTTCTATGATTTCTCGAGTTAATGATATACGCGCCTACCGAA ACCAAATACTTTTGGAACAGTCAGCTCTTACCACCACGCCCCGCCAACACCTTAATCCCAGAAATTGGCCCGCCGCC CTAGTATACCAGGAAAGTCCCGCTCCCACCACTGTATTACTTCCTCGAGACGCCCAGGCCGAAGTCCAAATGACTAA TGCAGGTGCGCAGTTAGCTGGCGGCTCCACCCTATGTCGTCACAGGCCTCGGCATAATATAAAACGCCTGATGATTA GAGGCCGAGGTATTCAGCTTAACGACGAGTCGGTGAGCTCTCCGCTTGGTCTACGACCAGACGGAATCTTTCAAATT GCCGGCTGCGGGAGATCTTCCTTCACCCCTCGTCAGACTGTTTTGACTTTGGAAAGTTCGTCTTCGCAACCCCGCTC GGGCGGAATCGGGACCGTTCAATTTGTGGAGGAGTTTACTCCCTCTGTCTACTTCAACCCTTTCTCCGGATCTCCTG GGCACTACCCGGACGAGTTCATACCGAACTTTGACGCAATTAGCGAGTCAGTGGACGGCTACGATTGATGTCTGGTG ACGCGGCTGAGCTATCTCGGCTGCGACATCTAGACCACTGCCGCCGCTTTCGCTGTTTTGCCCGGGAACTCATTGAG TTCATTTACTTCGAACTCCCCAAGGATCACCCTCAAGGTCCGGCCCACGGAGTGCGGATTACTATCGAAGGTAAAAT AAACTCTCGCCTGCATCGAATTTTCTCCCAGCGGCCCGTGCTGATCGAGCGAGACCAGGGAAACACCACGGTTTCTA TCTACTGCATTTGTAATCACCCAGGATTGCATGAAAGCCTTTGCTGTCTTATGTGTACTGAGTTTAATAAAAACTGA ATTAAGACTCTCCTACGGACTGCCGCTTCTTCAACCCGGATTTTACAACCAGAAGAACGAAACTTTTCCTCTCATCC AGGACTCTGTTAACTTTACCTTTCCTACTTACAAACCAGAAGCTCAACGACAACACCGCTTTTCCAGAAGCATTTTC CCTACTAATACTACTTTCAAAACCGGAGGTGAGCTCCACAGTCTCCCCGCAGAAAACCCTTGGGTGGAAGCGGGCCT TGTAGTGCTAGGAATTCTTGCGGGCGGGCTTGTGATTATTCTTTGCTACCTATACACACCTTGCTTCACTTTCCTAG TGGTGTTGTGGTATTGGTTTAAAAAATGGGGCCCATACTAGTCTTGCTTGTTTTACTTTCGCTTTTGGGACCGGGTT CTGCCAACTACAATCCATGTCTAGACTTTGACCCAGAAAACTGCACACTTACTTTTGCACCCGACACAAGCCGCATC TGTGGAGTTCTTATTAAGTGCGGATGGGAATGCAGGTCCGTTGAAATTACACACAATAACAAAACCTGGAACAATAC CTTATCCACCACATGGGAGCCAGGAGTTCCCGAGTGGTACACTGTCTCTGTCCGAGGTCCTGACGGTTCCATCCGCA TTAGTAACAACACTTTTATTTTTTCTACAATGTGCGATCTGGCCATGTTCATGAGCAAACAGTATTCTCTATGGCCT CCCAGCAAGGACAACATTGTAACGTTCTCCATTGCTTATTGCTTGTGCGCTTGCCTTCTTACTGCTTTACTGTGCGT ATGCATACACCTGCTTGTAACCACTCGTATCAAAAACGCCAATAACAAAGAAAAAATGCCTTAACCTCTTTCTGTTT ACAGACATGGCTTTTCTTACAGCTCTCATACTTGTCAGCATTGTCACTGCCGCTCACGGACAAACAGTCGTCTCTAT CCCTCTAGGTCATAATTACACTCTCATAGGACCCCCAATCACTTCAGAGGTCATCTGGACCAAACTGGGAAGCGTTG ATTACTTTGATATAATCTGTAACAAAACAAAACCAATAATAGTAACCTGCAACATACAAAATCTTACATTAATTAAT GTTAGCAAAGTTTACAGCGGTTACTATTATGGTTATGACAGATACAGTAGTCAATATAGAAATTACTTGGTTCGTGT TACCCAGTCCAAAACCACGAAAATGCCAAATATGGCAGAAATTCGATCCGATGACAATTCTCTAGAAACTTTTACAT CTTCCACCACACCTGACGAAAAAAATATCCCAGATTCAATGATTGCAATTATCGCAGCGGTGGCAGTGGTGATGGCA CTAACAGTAATATGCATGCTTTTATATGCTTGTCGCTACAAAAAGTTTCATCCTAAAAAACAAGATCTCCTACTAAG GCTTAACATTTAATTTCTTTTTACACAGCCATGGTTTCCACTACCACATTCCTTATGCTTACTAGTATAGCAACTCT GACTTCTGCTCGCTCACACCTCACTGTAACTATAGGCTCAAACTGCACACTAAAAGGACCTCAAGGTGGTCATGTCT TTTGGTGGAGAATATATGACAATGGATGGTTTACAAAACCATGTGACCAACCTGGTAGATTTTTCTGCAACGGCAGA GACCTAACCATTATCAACGTGACAGCAAATGACAAAGGCTTCTATTATGGAACCGACTATAAAAGTAGTTTAGATTA TAACATTATTGTACTGCCATCCACCACTCCAGCGCCCCGCAAAACTACTTTCTCTAGCAGCAGTGCCGCTAACAATA CAATTTCCAATCCAACCTTTACCGCGCTTTTAAAACGCACTGTGAATAATTCTACAACAATTTCCACTTCAACAATC AGCATCATCGCTGCCGTGACAATTGGAATATCTATTCTTGTTTTTACCATAACCTACTACACCTGCTGCTATAAAAA AGACGAACATAAAGGTGATCCATTACTTAGATTTGATATTTAATTTGTTCTTTTTTTTTTTATTTACAGTATGGTGA ACACCAATCATGGTACCTAGAAATTTCTTCTTCACCATACTCATCTGTGCTTTTAATGTTTGCGCTACTTTCACAGC AGTAGCCACAGCAAGCCCAGACTGTATAGGAGCATTTGCTTCCTATGCACTTTTTGCTTTTGTCACTTGCATCTGCG TATGTAGCATAGTCTGCCTGGTTATTAATTTTTTCCAACTTCTAGACTGGATCCTTGTGCGAATTGCCTACCTGCGC CACCATCCCGAATACCGCAACCAAAATATCGCGGCACTTCTTAGACTTATCTAAAACCATGCAGGCTATACTACCAA TATTTTTGCTTCTATTGCTTCCCTACGCTGTCTCAACCCCAGCTACCTATAGTACTCCACCAGAACACCTTAGAAAA TGCAAATTCCAACAACCGTGGTCATTTCTTGCTTGCTATCGAGAAAAATCTGAAATTCCCCCAACTTTAATAATGAT TGCTGGAATAATTAATGTAATCTGTTGCACCATAATTTCATTTCTGATATACCCCCTATTTGATTTTGGCTGGAATG CTCCCAATGCACATGATTATTCCCAAGACCCAGAGGAACACATTCCCCTACATAACATGCAACAACCAATAGCGCTA ATAGAATACGAAAGTGAACCACAACCCCCACTACTCCCTGCTATTAGTTACTTCAACCTAACCGGCGGAGATGACTG AAACACTCACCACCTCCAATTCCGCCGAGGATCTGCTTGATATGGACGGCCGCGTCTCAGAACAGCGACTCGCCCAA CTACGCATCCGCCAGCAGCAGGAACGCGTGGCCAAAGAGCTCAGAGATGTCATCCAAATTCACCAATGCAAAAAAGG CATATTCTGTTTGGTAAAACAAGCCAAAATATCCTACGAGATCACCGCTACCGACCATCGCCTCTCTTACGAGCTTG GCCCCCAACGACAAAAATTTACCTGCATGGTGGGAATCAACCCCATAGTTATCACCCAACAAAGTGGAGATACTAAG GGTTGCATTCACTGCTCCTGCGATTCCATCGAGTGCACCTACACCCTGCTGAAGACCCTATGCGGACTAAGAGACCT GCTACCCATGAATTAAAAAAATGATTAATAAAAAATCACTTACTTAAAATCAGCAATAAGGTCTCTATTGAAATTTT CTCCCAGCAGCACCTCACTTCCCTCTTCCCAACTCTGGTATTCTAAACCCCGTTCAGCGGCATACTTTCTCCATACT TTAAAGGGGATGTCAAATTTTAGCTCCTCTCCTGTACCCACAATCTTCATCTCTTTCTTCCCAGATGACCAAGAGAG TCCGGCTCAGTGACTCCTTCAACCCTGTCTACCCCTATGAAGATGAAAGCACCTCCCAACACCCCTTTATAAACCCA GGGTTTATTTCCCCAAATGGCTTCACCCAAAGCCCAGACGGAGTTCTTACTTTAAAATGTTTAACCCCGCTAACAAC CACAGGCGGGTCTCTACAGCTAAAAGTGGGAGGGGGACTTACAGTAGATGACACTGATGGGACCTTACAAGAAAACA TAGGTGCCACCACACCACTTGTTAAGACTGGGCACTCTATAGGTTTATCCCTAGGAGCCGGATTGGGAACAGATGAA AATAAACTTTGTACCAAATTGGGAGAAGGACTTACATTCAATTCAAACAACATTTGCATTGATGACAATATTAACAC CCTGTGGACAGGAGTTAACCCCACCGAAGCCAACTGTCAAATGATGGACTCCAGTGAATCTAATGATTGCAAATTAA TTCTAACACTAGTTAAAACTGGAGCCCTAGTCACTGCATTTGTTTATGTTATAGGAGTATCTAACAATTTTAATATG CTAACTACATACAGAAATATAAATTTTACTGCGGAGCTGTTTTTTGATTCTGCGGGTAATTTACTAACTAGCCTGTC ATCCCTAAAAACTCCACTTAATCATAAATCAGGACAAAACATGGCTACTGGTGCCATTACTAATGCTAAAAGTTTCA TGCCCAGCACAACTGCTTATCCTTTCAATAATAATTCTAGAGAAAAAGAAAACTACATTTACGGAACTTGTCACTAC ACAGCTAGTGATCACACTGCTTTTCCCATTGACATATCTGTCATGCTTAACCAAAGAGCAATAAGAGCTGATACATC ATATTGTATTCGTATAACTTGGTCCTGGAACACAGGAGATGCCCCAGAGGGGCAAACCTCTGCTACAACCCTAGTTA CCTCCCCATTTACCTTTTACTACATCAGAGAAGACGACTGACAAATAAAGTTTAACTTGTTTATTTGAAAATCAATT CACAAACTCCGAGTAGTTATTTTGCCTCCCCCTTCCCATTTAACAGAATATACCAATCTCTCCCCACGCACAGCTTT AAACATTTGGATACCATTAGAGATAGACATGGTTTTAGATTCCACATTCCAAACAGTTTCAGAGCGAGCCAATCTGG GGTCAGTGATAGATAAAAATCCATCGGGATAGTCTTTTAAAGCGCTTTCACAGTCCAACTGATGCGGATGCGACTCC GGAGTCTGGATCACGGTCATCTGGAAGAAGAACGATGGGAATCATAATCCGAAAACGGGATCGGGCGATTGTGTCTC ATCAAACCCACAAGCAGCCGCTGTCTGCGTCGCTCCGTGCGACTGCTGTTTATGGGATCGGGATCCACAGTGTCCTG AAGCATGATTTTAATAGCCCTTAACATTAACTTTCTGGTGCGATGCGCGCAGCAACGCATTCTTATTTCACTTAGAT TAATACAGTAGGTACAGCACATTATCACAATATTGTTTAATAAACCATAATTAAAAGCACTCCAGCCAAAACTCATA TCTGATATAATCGCCCCTGCATGACCATCATACCAAAGTTTAATATAAATTAAATGACGTTCCCTCAAAAACACACT ACCCACATACATGATCTCTTTTGGCATGTGCATATTAACAATCTGTCTGTACCATGGACAACGTTGGTTAATCATGC AGCCCAATATAACCTTCCGGAACCACACTGCCAACACCGCTCCCCCAGCCATGCATTGAAGTGAACCCTGCTGATTA CAATGACAATGAAGAATCCAATTCTCTCGACCGTGAATCACTTGAGAATGAAAAATATCTATAGTAGCACAACATAG ACATAAATGCATGCATCTTCTCATAATTTTTAACTCCTCAGGATTTAGAAACATATCCCAAGGAATAGGAAGCTCTT GCAGAACAGTAAAGCTGGCAGAACAAGGAAGACCACGAACACAACTTACACTATGCATAGTCATAGTATCACAATCT GGCAACAGCGGGTGGTCTTCAGTCATAGAAGCTCGGGTTTCATTTTCCTCACAACGTGGTAACTGGGCTCTGGTGTA AGGATGATGTCTGGCGCATGATGTCGAGCGTGCGCACAACCTTGTCATAATGGAGTTGCTTCCTGACATTCTCGTAT TTTGTATAGCAAAACGCGGCCCTGGCAGAACACACTCTTCTTCGCCTTTTATCCTGCCGCTTAGCGTGTTCCGTGTG ATAGTTCAAGTACAGCCACACTCTTAAGTTGGTCAAAAGAATGCTGGCTTCAGTTGTAATCAAAACTCCATCACATC TAATCGTTCTGAGGAAATGATCCACGGTAGCATATGCAAATCCCAACCAAGCAATGCAACTGGATTGCGTTTCAAGC AGGAGAGGAGAGGGAAGAGACGGAAGAACCATGTTAATTTTTATTCCAAACGATCTCGCAGTACTTCAAATTGTAGA TCGCGCAGATGGCATCTGTCGCCCCCACTGTGTTGGTGAAAAAGCACAGCTAGATCAAAAGAAATGCGATTTTCAAG GTGCTCAACGGTGGCTTCCAGCAAAGCCTCCACGCGCACATCCAAGAACAAAAGAATACCAAAAGAAGGAGCATTTT CTAACTCCTCAATCATCATATTACAGTCCTGCACCATTCCCAGATAATTTTCAGCTTTCCAGCCTTGGATTATTCGT GTCATTTCTTGTGGTAAATCCAATCCACACATTACAAACAGGTCCCGGAGGGCGCCCTCCACCACCATTCTTAAACA CACCCTCATAATGACAAAATATCTTGCTCCTGTGTCACCTGTAGCAAATTGAGAATGGCAACATCAATTGACATGCC GTTGGCTCTAAGTTCTTCTCTAAGTTCTAGTTGTAAATACTCTTTCATATTATCACCAAACTGCTTAGCCAGAAGCC CCCCGGGAACAAGAGCAGGACACGCTACAGTGCAGTACAAGCGCAGACCTCCCCAATTGGCTCCAGCAAAAACAAGA TTGGAATAAGCATATTGGGAACCACCAGTAATGTCATCAAAGTTGCTGGAAATATAATCAGGCAGAGTTTGTTGTAA AAATTGAATAAAAGAAAAATTTGCCAAAAAAACATTCAAAACCTCTGGGATGCAAATGCAATAAGTTACCGCGCTGC GCTCCAACATTGTTAGTTTTGAATGAGTCTGCAAAAAATAAAAAAACAAGCGTCATATCAGAGTAGCCTGACGAACA GGTGGATAAATCAGTCTTTCCATCACAAGACAAGCCACAGGGTCTCCAGCCCGACCCTCGTAAAACCTGTCATCGTG ATTAAACAACAGCACCGAAAGTTCCTCGCGGTGACCAGCATGAATAATTCTTGATGAAGCATACAATCCAAACATGT TAGCATCAGTTAAAGACAAAAAACAGCCAATATAGCCTCTGGGTATAATTATGCTTAATCGTAAATATAGCAAAGCC ACCCCTCGCGGATACAAAGTAAAAGGCACAGGAGAATAAAAAATATAATTATTCCTTTGCTGCTGTTCAGGCAACGT CGCCCCCGGTCCCTCTAAATACACATACAAAGCCTCATCAGCCATGGCTTACCAGACAAAGTACAGCAGGCACACAA AGCACAAGCTCTAAAGTCACTCACCAACCTGTCCACAGTATATATACACAAACCCTAAACTGACGTAATGGGGCTAA AGTACACAAAATCCCGCCAAACCCAACACACACCCCGAAACTGCGTCACCACAAAAGTACAGTTTCACTTCCGCAAT CCCAACAAGCGGCACTTCCTCTTTCTCACGGGACGTCACATCCGCTTAACTTGCAACCTCATTTTCCCACGGCCGCG CCGCCCCTTTTAGCCGTTAACCCCACAGCCAATTACCACACAGCCCACACTTTTTAAAATCACCTCATTTACATATT GGCACCATTCCATCTATAAGGTATATTATTGATGATG

GenBank Accession No. AAW33140 MTKRVRLSDSFNPVYPYEDESTSQHPFINPGFISPNGFTQSPDGVLTLKCLTPLTTTGGSLQLKVGGGLTVDDTDGT LOENIGATTPLVKTGHSIGLSLGAGLGTDENKLCTKLGEGLTENSNNICIDDNINTLWTGVNPTEANCOMMDSSESN DCKLILTLVKTGALVTAFVYVIGVSNNENMLTTYRNINFTAELFFDSAGNLLTSLSSLKTPLNHKSGONMATGAITN AKSFMPSTTAYPENNNSREKENYIYGTCHYTASDHTAFPIDISVMLNQRAIRADTSYCIRITWSWNTGDAPEGQTSA TTLVTSPFTFYYIREDD

GenBank Accession No. AAW33119 MRRVVLGGAVVYPEGPPPSYESVMQQQQATAVMOSPLEAPFVPPRYLAPTEGRNSIRYSELAPQYDTTRLYLVDNKS ADIASLNYQNDHSNFLTTVVONNDFTPTEASTQTINFDERSRWGGQLKTIMHTNMPNVNEYMESNNFKARVMVSRKP PEGAAVGDTYDHKQDILEYEWFEFTLPEGNFSVTMTIDLMNNAIIDNYLKVGRONGVLESDIGVKFDTRNFKLGWDP ETKLIMPGVYTYEAFHPDIVLLPGCGVDFTESRLSNLLGIRKKOPFQEGFKILYEDLEGGNIPALLDVDAYENSKKE QKAKIEAAAEAKANIVASDFTRVANAGEVRGDNFAPTPVPTAESLLADVTGGTDVKLTIQPVEKDSKNRSYNVLEDK INTAYRSWYLSYNYGDPEKGVRSWTLLTTSDVTCGAEQVYWSLPDMMQDPVTFRSTRQVSNYPVVGAELMPVFSKSF YNEQAVYSQQLRQSTSLTHVENREPENQILIRPPAPTITTVSENVPALTDHGTLPLRSSIRGVQRVTVTDARRRTCP YVYKALGIVAPRVLSSRTF

GenBank Accession No. AAW33124 MATPSMLPQWAYMHIAGQDASEYLSPGLVQFARATDTYENLGNKERNPTVAPTHDVTTDRSQRLMLRFVPVDREDNT YSYKVRYTLAVGDNRVLDMASTFFDIRGVLDRGPSFKPYSGTAYNSLAPKGAPNASQWLDKGVETTEERQNEDGEND EKATYTFGNAPVKADADITKDGLPIGLEVPAEGDPKPIYANKLYQPEPQVGQESWTDTDGTEEKYGGRVLKPDTKMK PCYGSFAKPTNVKGGQAKVKTEEGNNIEYDIDMNFFDLRSQKOGLKPKIVMYAENVDLESPDTHVVYKPEVSDASSN ANLGQQSMPNRPNYIGFRDNFIGLMYYNSTGNMGVLAGQASQLNAVVDLQDRNTELSYQLLLDSLGDRTRYFSMWNQ AVDSYDPDVRVIENHGVEDELPNYCFPLDGIGPRTDSYKEIQLNGDQAWKDVNPNGISELVKGNPFAMEINLQANLW RSFLYSNVALYLPDSYKYTPSNVTLPENKNTYDYMNGRVVPPSLVDTYVNIGARWSLDAMDNVNPFNHHRNAGLRYR SMLLGNGRYVPFHIQVPQKFFAVKNLLLLPGSYTYEWNFRKDVNMVLQSSLGNDLRVDGASISFTSINLYATFFPMA HNTASTLEAMLRNDTNDQSFNDYLSAANMLYPIPANATNIPISIPSRNWAAFRGWSFTRLKTKETPSLGSGFDPYFV YSGSIPYLDGTFYLNHTFKKVSIMFDSSVSWPGNDRLLSPNEFEIKRTVDGEGYNVAQCNMTKDWELVOMLANYNIG YQGFYIPEGYKDRMYSFFRNFQPMSROVVDEVNYKDFKAVAIPYQHNNSGFVGYMAPTMRQGQPYPANYPYPLIGTT AVNSVTQKKFLCDRTMWRIPFSSNFMSMGALTDLGONMLYANSAHALDMTFEVDPMDEPTLLYLLFEVFDVVRVHQP HRGIIETVYLRTPFSAGNATT

GenBank Accession No. AY601636 (SEQ ID NO: 213) CATTATCTATAATATACCTTATAGATGGAATGGTGCCAACATGTAAATGAGGTAATTTAAAAAAGTGCGCGCTGTGT GGTGATTGGCTGCGGGGTGAACGGCTAAAAGGGGGGGGCAATGCTGGGATGTGACGTAACTTATGTGGGAGGAGTTA TGTTGCAAGTTATCGCGGTAAAGGTGACGTAAAACGAGGTGTGGTTTGGACACGGAAGTAGACAGTTTTCCCACGTT TACTGACAGGATATGAGGTAGTTTTGGGCGGATGCAAGTGAAAATTCTCCATTTTCGCGCGAAAACTGAATGAGGAA GTGAATTTCTGAGTCATTTCGCGGTTATGACAGGGTGGAGTATTTGCCGAGGGCCGAGTAGACTTTGACCGTTTACG TGGAGGTTTCGATTACCGTGTTTTTCACCTAAATTTCCGCGTACGGTGTCAAAGTCCTGTGTTTTTACGTAGGTGTC AGCTGATCACTAGGGTATTTAAACCTGTCGAGTTCCGTCAAGAGGCCACTCTTGAGTGCCAGCGAGAAGAGTTTTCT CCTCCGCGCTGCGAGTCAGTTTTGCGCTTTGAAAATGAGACACCTGCGATTCCTGCCACAGGAGATTATCTCCAGCG AGACCGGGATCGAAATACTGGAGTTTGTGGTAAATACCCTGATGGGAGATGACCCGGAACCGCCAGTGCAGCCTTTC GATCCACCTACGCTGCACGATCTGTATGATTTAGAGGTAGACGGGCCTGATGATCCCAATGAGGAAGCTGTAAATGG GTTTTTTACTGATTCTATGCTGCTAGCTGCCGATGAAGGATTGGACATAAACCCTCCTCCTGAGACCCTTGATACCC CAGGGGTGGTTGTGGAAAGCGGCAGAGGTGGGAAAAAATTGCCTGATCTGGGAGCAGCTGAAATGGACTTGCGTTGT TATGAAGAGGGTTTTCCTCCGAGTGATGATGAAGACGGGGAAACTGAACAGTCCATCCATACCGCAGTGAATGAGGG AGTAAAAGCTGCCAGCGATGTTTTTAAGTTGGACTGTCCGGAGCTGCCTGGACATGGCTGTAAGTCTTGTGAATTTC ACAGGAATAACACTGGAATGAAAGAACTATTGTGCTCGCTTTGCTATATGAGAATGCACTGCCACTTTATTTACAGT AAGTGTATTTAAGTGAAATTTAAAGGAATAGTGTAGCTGTTTAATAACTGTTGAATGGTAGATTTATGTTTTTGCTT GCGATTTTTTGTAGGTCCTGTGTCTGATGATGAGTCACCTTCTCCTGATTCAACTACCTCACCTCCTGAAATTCAGG CGCCCGTACCTGCAAACGTATGCAAGCCCATTCCTGTGAAGCCTAAGTCTGGGAAACGCCCTGCTGTGGATAAGCTT GAGGACTTGTTGGAGGGTGGGGATGGACCTTTGGACCTTAGTACCCGGAAACTGCCAAGGCAATGAGTGCCCTGCAG CTGTGTTTATTTAGTGACGTCATGTAATAAAATTATGTCAGCTGCTGAGTGTTTTATTGCTTCTTGGGTGGGGACTT GGATATATAAGTAGGAGCAGATCTGTGTGGTTAGCTCATAGCAACCTGCTGCCATCCATGGAGGTTTGGGCTATCTT GGAAGACCTGAGACAGACTAGGCTACTGCTAGAAAACGCCTCGGACGGAGTCTCTGGCTTTTGGAGATTCTGGTTCG GTGGCGATCTAGCTAGGCTAGTGTTTAGGATAAAACAGGACTATAGGGAAGAATTTGAAAAGTTATTGGACGACAGT CCAGGACTTTTTGAAGCTCTTAACTTGGGCCATCAGGCTCATTTTAAGGAGAAGGTTTTATCAGTTTTAGATTTTTC TACTCCTGGTAGAACTGCTGCTGCTGTAGCTTTTCTTACTTTTATATTGGATAAATGGATCCGCCAAACCCACTTCA GCAAGGGATACGTTTTGGATTTCATAGCAGCAGCTTTGTGGAGAACATGGAAGGCTCGCAGGATGAGGACAATCTTA GATTACTGGCCAGTGCAGCCTCTGGGAGTAGCAGGGATACTGAGACACCCACCGGCCATGCCAGCGGTTCTGGAGGA GGAGCAGCAGGAGGACAATCCGAGAGCCGGCCTGGACCCTCCGGTGGAGGAGTAGCTGACTTGTTTCCTGAACTGCG ACGGGTGCTTACTAGGTCTACGTCCAGTGGACAGGACAGGGGCATTAAGAGGGAAAGGAATCCTAGTGGGAATAATT CAAGAACCGAGTTGGCTTTAAGTTTAATGAGCCGTAGGCGTCCTGAAACTGTTTGGTGGCATGAGGTTCAGAGCGAA GGCAGGGATGAAGTTTCAATATTGCAGGAGAAATATTCACTAGAACAACTTAAGACCTGTTGGTTGGAACCTGAGGA TGATTGGGAGGTGGCCATTAGGAATTATGCTAAGATATCTCTGAGGCCTGATAAACAGTATAGAATTACTAAGAAGA TTAATATTAGAAATGCATGCTACATATCAGGGAATGGGGCAGAGGTTATAATAGATACACAAGATAAAGCAGCTTTT AGATGTTGTATGATGGGTATGTGGCCAGGGGTTGTCGGCATGGAAGCAGTAACATTTATGAATATTAGGTTTAAAGG GGATGGGTATAATGGCATTGTATTTATGGCTAACACTAAGCTGATTCTACATGGTTGTAGCTTTTTTGGGTTTAATA ATACTTGTGTAGAAGCTTGGGGGCAAGTTAGTGTGAGGGGTTGTAGTTTTTATGCATGCTGGATTGCAACATCAGGT AGGGTCAAGAGTCAGTTGTCTGTGAAGAAATGCATGTTTGAGAGATGTAATCTTGGCATACTGAATGAAGGTGAAGC AAGGGTCCGCCACTGCGCAGCTACAGAAACTGGCTGCTTCATTCTAATAAAGGGAAATGCCAGTGTGAAGCATAATA TGATCTGTGGACATTCGAATGAGAGGCCTTATCAGATGCTGACCTGCGCTGGTGGACATTGCAATATTCTGGCTACC GTGCATATCGTTTCCCATGCACGCAAGAAATGGCCTGTATTTGAACATAATGTGATTACCAAGTGCACCATGCACAT AGGTGGTCGCAGGGGAATGTTTATGCCTTACCAGTGTAACATGAATCATGTGAAGGTGATGTTGGAACCAGATGCCT TTTCCAGAGTAAGCTTAACAGGAATCTTTGATATGAATATTCAACTATGGAAGATCCTGAGATATGATGACACTAAA CCGAGGGTGCGCGCATGCGAATGCGGAGGCAAGCATGCTAGATTCCAGCCGGTGTGCGTGGATGTGACTGAAGACCT GAGACCCGATCATTTGGTGCTTGCCTGCACTGGAGCGGAGTTCGGTTCTAGTGGTGAAGAAACTGACTAAAGTGAGT AGTGGGACGAGCTGTGGAGGTGGGACTTTGAGGTTGGTAAGGTGGGCAGATTGGGTAAATTTTGTTAATTTCTGTCT TGCAGCTGCCATGAGTGGAAGCGCTTCTTTTGAGGGGGGAGTATTTAGCCCTTATCTGACGGGCAGGCTCCCACCAT GGGCAGGAGTTCGTCAGAATGTCATGGGATCCACTGTGGATGGGAGACCCGTCCAGCCCGCCAATTCCTCAACGCTG ACCTATGCCACTTTGAGTTCGTCACCATTGGATGCAGCTGCAGCCGCCGCCGCTACTGCTGCCGCCAACACCATCCT TGGAATGGGCTATTACGGAAGCATCGTTGCCAATTCCAGTTCCTCTAATAACCCTTCAACCCTGGCTGAGGACAAGC TACTTGTTCTCTTGGCTCAGCTCGAGGCCTTAACCCAACGCTTAGGCGAACTGTCTAAGCAGGTGGCCCAGTTGCGT GAGCAAACTCAGTCTGCTGTTGCCACAGCAAAGTCTAAATAAAGATCTTAAATCAATAAATAAAGAAATACTTGTTA TAAAAACAAATGAATGTTTATTTGATTTTTCGCGCGCGGTATGCCCTGGACCATCGGTCTCGATCATTGAGAACTCG GTGGATCTTTTCCAGTACCCTGTAAAGGTGGGATTGAATGTTTAGATACATGGGCATTAGTCCGTCCCGGGGGTGGA GATAGCTCCATTGAAGAGCCTCTTGCTCCGGGGTAGTGTTATAAATCACCCAGTCATAGCAAGGTCGGAGTGCATGG TGTTGCACAATATCTTTTAGGAGCAGACTAATTGCAACGGGGAGGCCCTTAGTGTAGGTGTTTACAAATCTGTTGAG CTGGGACGGGTGCATCCGGGGTGAAATTATATGCATTTTGGACTGGATCTTGAGGTTGGCAATGTTGCCGCCTAGAT CCCGTCTGGGGTTCATATTGTGCAGAACCACCAAGACAGTGTATCCGGTGCACTTGGGAAATTTATCATGCAGCTTA GAGGGAAAAGCATGAAAAAATTTGGAGACGCCTTTGTGACCCCCCAGATTCTCCATGCACTCATCCATAATGATAGC GATGGGGCCGTGGGCAGCGGCACGGGCGAACACGTTCCGGGGGTCTGAAACATCATAGTTATGCTCCTGAGTCAGGT CATCATAAGCCATTTTAATAAACTTTGGGCGAAGGGTGCCAGATTGGGGGATGAAAGTTCCCTCTGGCCCGGGAGCA TAGTTTCCCTCACATATTTGCATTTCCCAGGCTTTCAGTTCAGAGGGGGGGATCATATCCACCTGCGGGGCTATAAA AAATACTGTTTCTGGAGCCGGGGTGATTAACTGGGATGAGAGCAAATTCCTAAGCAGCTGAGACTTGCCGCACCCAG TGGGACCGTAAATGACCCCAATTACGGGTTGCAGATGGTAGTTTAGGGAGCGACAGCTGCCGTCCTCCCGGAGCAGG GGGGCCACTTCGTTCATCATTTCCCTTACATGGATATTTTCCCGCACCAAGTCCGTTAGGAGGCGCTCTCCCCCAAG GGATAGAAGCTCCTGGAGCGAGGAGAAGTTTTTCAGCGGCTTCAGCCCGTCAGCCATGGGCATTTTGGAAAGAGTCT GTTGCAAGAGCTCGAGCCGGTCCCAGAGCTCGGTGATGTGCTCTATGGCATCTCGATCCAGCAGACCTCCTCGTTTC GCGGGTTGGGACGGCTCCTGGAGTAGGGAATCAGACGATGGGCGTCCAGCGCTGCCAGGGTCCGATCCTTCCATGGT CGCAGCGTCCGAGTCAGGGTTGTTTCCGTCACGGTGAAGGGGTGCGCGCCTGGTTGGGCGCTTGCGAGGGTGCGCTT CAGACTCATCCTGCTGGTCGAGAACCGCTGCCGATCGGCGCCCTGCATGTCGGCCAGGTAGCAGTTTACCATGAGTT CGTAGTTGAGCGCCTCGGCCGCGTGGCCTTTGGCACGGAGCTTACCTTTGGAAGTTTTATGGCAGGCGGGGCAGTAG ATACATTTGAGGGCATACAGCTTGGGCGCGAGGAAAATGGATTCGGGGGAGTATGCATCCGCACCGCAGGAGGCGCA GACGGTTTCGCACTCCACGAGCCATGTCAGATCCGGCTCATCGGGGTCAAAAACAAGTTTTCCGCCATGTTTTTTGA TGCGTTTCTTACCTTTGGTTTCCATGAGTTCGTGTCCCCGCTGGGTAACAAAGAGGCTGTCCGTGTCCCCGTAGACT GACTTTATGGGCCTGTCCTCGAGCGGAGTGCCGCGGTCCTCTTCGTAGAGGAACCCAGCCCACTCTGATACAAAAGC GCGTGTCCAGGCCAGCACAAAGGAGGCCACGTGGGAGGGGTAGCGGTCGTTGTCAACCAGGGGGTCCACCTTCTCTA CGGTATGTAAACACATGTCCCCCTCCTCCACATCCAAGAATGTGATTGGCTTGTAAGTGTAGGCCACGTGACCAGGG GTCCCCGCCGGGGGGGTATAAAAGGGGGGGGCCTCTGTTCGTCTTCACTGTCTTCCGGATCGCTGTCCAGGAGCGC CAGCTGTTGGGGTAGGTATTCCCTCTCGAAGGCGGGCATGACCTCTGCACTCAGGTTGTCAGTTTCTAGGAACGAGG AGGATTTGATATTGACAGTACCAGCAGAGATGCCTTTCATAAGACTCTCGTCCATCTGGTCAGAAAACACAATCTTC TTGTTGTCCAGCTTGGTGGCAAATGATCCATAGAGGGCATTGGATAGAAGCTTGGCGATAGAGCGCATGGTTTGGTT CTTTTCCTTGTCCGCGCGCTCCTTGGCGGCGATGTTAAGCTGGACGTACTCGCGCGCCACACATTTCCATTCAGGGA AGATGGTTGTCAGTTCATCCGGAACTATTCTGACTCGCCATCCCCTATTGTGCAGGGTTATCAGATCCACACTGGTG GCTACCTCGCCTCGGAGGGGCTCATTGGTCCAGCAGAGTCGACCTCCTTTTCTTGAACAGAAAGGGGGGAGGGGGTC TAGCATGAACTCATCAGGGGGGTCCGCATCTATGGTAAATATTCCCGGTAGCAAATCCTTGTCAAAATAGCTGATGG TGGCGGGATCATCCAAAGTCATCTGCCATTCTCGAACTGCCAGCGCGCGCTCATAGGGGTTAAGAGGGGTGCCCCAG GGCATGGGGTGGGTGAGCGCGGAGGCATACATGCCACAGATATCGTAGACATAGAGGGGCTCTTCGAGGATGCCGAT GTAAGTGGGATAACAGCGCCCCCCTCTGATGCTTGCTCGCACATAGTCATAGAGTTCATGTGAGGGGGCGAGAAGAC CCGGGCCCAGATTGGTGCGGTTGGGTTTTTCCGCCCTGTAAACGATCTGGCGAAAGATGGCATGGGAATTGGAAGAG ATAGTAGGTCTCTGGAATATGTTAAAATGGGCATGAGGGAGGCCTACAGAGTCCCTTATGAAGTGGGCATATGACTC TTGCAGCTTGGCTACCAGCTCGGCGGTGACAAGTACGTCTAGGGCACAGTAGTCGAGAGTTTCCTGGATGATGTCAT AACGCGGTTGGTTTTTCTTTTCCCACAGCTCGCGGTTGAGAAGGTATTCTTCGCGATCCTTCCAGTACTCTTCGAGG GGAAACCCGTCTTTTTCTGCACGGTAAGAGCCCAACATGTAGAATTGATTGACTGCCTTGTAGGGACAGCATCCCTT CTCCACTGGGAGAGAGTATGCTTGGGCTGCATTGCGCAGCGAGGTATGAGTGAGGGCAAAAGTGTCCCTGACCATGA CTTTGAGGAATTGATACTTGAAGTCGATGTCATCACAGGCCCCCTGTTCCCAGAGTTGGAAGTCCACCCGCTTCTTG TAGGCGGGGTTGGGCAAAGCGAAAGTAACATCATTGAAGAGGATCTTGCCGGCCCTGGGCATAAAATTTCGGGTGAT TCTGAAAGGCTGAGGGACCTCTGCTCGGTTATTGATAACCTGAGCGGCCAAGACGATCTCATCAAAGCCATTGATGT TGTGCCCCACTATGTACAGTTCTAAGAATCGAGGTGTGCCCCTGACATGAGGCAGCTTCTTGAGTTCTTCAAAAGTG AGGTCTGTAGGGTCAGTGAGAGCATAGTGTTCGAGGGCCCATTCGTGCAGGTGAGGGTTCGCTTTGAGGAAGGAGGA CCAGAGGTCCACTGCCAGTGCTGTTTGTAACTGGTCCCGGTACTGACGAAAATGCTGCCCGACTGCCATCTTTTCTG GGGTGACGCAATAGAAGGTTTGGGGGTCCTGCTGCCAGCGATCCCACTTGAGTTTTATAGCCAGGTCATAGGCGATG TTGACGAGCCGCTGGTCTCCAGAGAGTTTCATGACCAGCATGAAGGGGATTAGCTGCTTGCCAAAGGACCCCATCCA GGTGTAGGTTTCCACATCGTAGGTGAGGAAGAGCCTTTCTGTGCGAGGATGAGAGCCAATCGGGAAGAACTGGATCT CCTGCCACCAGTTGGAGGAATGGCTGTTGATGTGATGGAAGTAGAACTCCCTGCGACGCGCCGAGCATTCATGCTTG TGCTTGTACAGACGGCCGCAGTACTCGCATCGATTCACGGGATGCACCTCATGAATGAGTTGTACCTGACTTCCTTT GACGAGAAATTTCAGTGGAAAATTGAGGCCTGGCGCTTGTACCTCGCGCTCTACTATGTTGTCTGCATCGGCATGAC CATCTTCTGTCTCGATGGTGGTCATGCTGACGAGCCCTCGCGGGAGGCAAGTCCAGACCTCGGCGCGGCAGGGGCGG AGCTCGAGGACGAGAGCGCGCAGGTCGGAGCTGTCCAGGGTCCTGAGACGCTGCGGAGTCAGGTTAGTAGGCAGTGT CAGGAGATTGACTTGCATGATCTTTTCGAGGGCGTGAGGGAGGTTCAGATGGTACTTGATCTCCACGGGTCCGTTGG TGGAGATGTCGATGGCTTGCAGGGTTCCGTGCCCCTTGGGCGCTACCACCGTGCCCTTGTTTTTCCGTTTGGGCGGC GGTGGCTCTGTTGCTTCTTGCATGTTTAGAAGCGGTGTCGAGGGCGCGCACCGGGCGGCAGGGGCGGCTCGGGACCC GGCGGCATGGCCGGCAGTGGTACGTCGGCGCCGCGCGCGGGTAGGTTCTGGTACTGCGCCCTGAGAAGACTTGCATG CGCGACGACGCGGCGGTTGACATCCTGGATCTGACGCCTCTGGGTGAACGCTACCGGCCCCGTGAGCTTGAACCTGA AAGAGAGTTCAACAGAATCAATCTCGGTATCGTTGACGGCGGCTTGCCTAAGGATTTCTTGCACGTCGCCAGAGTTG TCCTGGTAGGCGATCTCGGCCATGAACTGCTCGATCTCTTCCTCTTGAAGATCTCCGCGGCCCGCTCTCTCGACGGT GGCCGCAAGGTCGTTGGAGATGCGTCCAATGAGTTGAGAGAATGCATTCATGCCCGCCTCGTTCCAGACGCGGCTGT AGACCACAGCCCCCTCGGGATCTCTCGCGCGCATGACCACCTGGGCGAGGTTGAGCTCCACGTGGCGGGTGAAGACC GCATAGTTGCATAGGCGCTGGAAAAGGTAGTTGAGTGTGGTGGCGATGTGCTCGGTGACGAAGAAATACATGATCCA TCGTCTCAGCGGCATCTCGCTAACATCGCCCAGCGCTTCCAAGCGCTCCATGGCCTCGTAGAAGTCCACGGCAAAGT TGAAAAACTGGGAGTTACGCGCGGACACGGTCAACTCCTCTTCCAGAAGACGGATGAGTTCGGCGATGGTGGTGCGC ACCTCGCGCTCGAAAGCCCCCGGGATTTCTTCCTCAATTTCTTCTTCTTCCACTAACATCTCTTCCTCTTCAGGTGG GGCTGCAGGAGGAGGGGGAACGCGGCGACGCCGGCGGCGCACAGGCAGACGGTCGATGAATCTTTCAATGACCTCTC CGCGGCGGCGGCGCATGGTCTCGGTGACGGCACGACCGTTCTCCCTGGGTCTCAGAGTGAAGACGCCTCCGCGCATC TCCCTGAAGTGGTGACTGGGAGGCTCTCCGTTGGGCAGGGACACCGCGCTGATTATGCATTTTATCAATTGCCCCGT AGGTACTCCGCGCAAGGACCTGATCGTCTCAAGATCCACGGGATCTGAAAACCTTTCGACGAAAGCGTCTAACCAGT CGCAATCGCAAGGTAGGCTGAGCACTGTTTCTTGCGGGGGGGGCGGCTAGACGCTCGGTCGGGGTTCTCTCTTTCT TCTCCTTCCTCCTCTTTGGAGGGTGAGACGATGCTGCTGGTGATGAAATTAAAATAGGCAGTTTTGAGACGGCGGAT GGTGGCGAGGAGCACCAGGTCTTTGGGTCCGGCTTGTTGGATGCGCAGGCGATGGGCCATTCCCCAAGCATTATCCT GACATCTGGCCAGATCTTTATAGTAGTCTTGCATGAGTCGTTCCACGGGCACTTCTTCTTCGCCCGCTCTGCCATGC ATGCGAGTGATCCCGAACCCGCGCATGGGCTGGACAAGTGCCAGGTCCGCTACAACCCTTTCTGCGAGGATGGCTTG CTGCACCTGGGTGAGGGTGGCTTGGAAGTCGTCAAAGTCCACGAAGCGGTGGTAAGCCCCGGTGTTGATTGTGTAGG AGCAGTTGGCCATGACTGACCAGTTGACTGTCTGGTGCCCCGGACGCACAATCTCGGTGTACTTGAGGCGCGAGTAG GCGCGGGTGTCAAAGATGTAATCGTTACAGGTGCGCACCAGGTACTGGTAGCCGATGAGAAAGTGAGGCGGCGGCTG GCGGTATAGGGGCCATCGCTCTGTAGCCGGGGCGCCAGGGGCGAGGTCTTCCAGCATGAGGCGGTGATAACCGTAGA TGTACCTGGACATCCAGGTGATACCGGAGGCGGTGGTGGATGCCCGCGGGAACTCGCGTACGCGGTTCCAGATGTTG CGCAGCGGCATGAAGTAGTTCATGGTAGGCACGGTTTGGCCCGTGAGACGCGCACAGTCGTTGATGCTCTAGACATA CGGGCAAAAACGAAAGCGGTCAGCGGCTCGTCTCCGTGGCCTGGAGGCTAAGCGAACGGGTTGGGCTGCGCGTGTAC CCCGGTTCGAATCTCGGATCAGGCTGGAGCCGCAGCTAACGTGGTACTGGCACTCCCGTCTCGACCCAGGCCTGCAC AAAACCTCCAGGATACGGAGGCGGGTCGTTTTTTTTTTGCTTTTTCCTGGATGGGAGCCAGTGCTGCGTCAAGCTTT AGAACACTCAGTTCTCGGGGCTGGGAGTGGCTCGCGCCCGTAGTCTGGAGAATCAATCGCCAGGGTTGCGTTGCGGT GTGCCCCGGTTCGAGTCTTAGCGCGCCGGATCGGCCGGTTTCCGCGACAAGCGAGGGTTTGGCAGCCTCGTCATTTC TAAGACCCCGCCAGCCGACTTCTCCAGTTTACGGGAGCGAGCCCTCTTTTTTTTGTTTTTTGTTGCCCAGATGCATC CCGTGCTGCGACAGATGCGCCCCCAGCAACAGCCCCCTTCTCAGCAGCAGCTACAACAACAGCCACAAAAGGCTCTT CCTGCTCCTGTAACTACTGCGGCTGCAGCCGTCAGCGGCGCGGGACAGCCCGCCTATGATCTGGACTTGGAAGAGGG CGAGGGATTGGCGCGCCTGGGGGCTCCATCGCCCGAGCGGCACCCGCGGGTGCAACTAAAAAAGGACTCTCGCGAGG CGTACGTGCCCCAACAGAACCTATTCAGGGACAGGAGCGGCGAGGAGCCAGAGGAGATGCGAGCATCTCGATTTAAC GCGGGTCGCGAGCTGCGCCACGGTCTGGATCGAAGACGGGTGCTGCAAGACGAGGATTTTGAGGTCGATGAAGTGAC AGGGATCAGCCCAGCTAGGGCACATGTGGCCGCGGCCAACCTAGTCTCGGCCTACGAGCAGACCGTGAAGGAGGAGC GCAACTTCCAAAAATCTTTTAACAACCATGTGCGCACCCTGATCGCCCGCGAGGAAGTGACCCTGGGTCTGATGCAC CTGTGGGACCTGATGGAGGCTATCACCCAGAACCCCACTAGCAAACCCCTGACAGCTCAGCTGTTTCTGGTGGTTCA ACATAGCAGGGACAACGAGGCATTCAGGGAGGCGTTGTTGAATATCACCGAGCCTGATGGGAGATGGCTGTATGATC TGATTAACATCCTGCAAAGTATTATAGTGCAGGAACGTAGCCTGGGTTTGGCTGAGAAAGTGGCAGCTATCAACTAC TCGGTCTTGAGCCTGGGCAAATACTACGCTCGCAAGATCTACAAGACCCCCTACGTACCCATTGACAAGGAGGTGAA GATAGATGGGTTTTACATGCGCATGACTCTCAAGGTGTTGACTTTAAGCGACGATCTGGGGGTGTATCGCAATGACA GGATGCACCGCGCGGTGAGCGCCAGCAGGAGGCGCGAGCTGAGCGACAGAGAACTTATGCACAGCTTGCAAAGGGCT CTAACGGGGGCCGGAACTGATGGGGAGAACTACTTTGACATGGGAGCGGACTTGCAATGGCAACCCAGTCGCAGGGC CATGGAGGCTGCAGGGTGTGAGCTTCCTTACATAGAAGAGGTGGATGAAGTCGAGGACGAGGAGGGCGAGTACTTGG AAGACTGATGGCGCGACCCGTATTTTTGCTAGATGGAACAACAGCAGGCACCGGACCCCGCAATGCGGGCGGCGCTA CAGAGCCAGCCGTCCGGCATTAACTCCTCGGACGATTGGACCCAGGCCATGCAACGTATAATGGCGCTGACGACCCG CAACCCCGAAGCCTTTAGACAGCAACCCCAGGCCAACCGCCTTTCGGCCATACTGGAGGCCGTAGTGCCCTCCCGCT CCAACCCCACCCACGAGAAGGTCCTGGCTATCGTGAACGCGCTGGTGGAGAACAAGGCCATCCGTCCCGATGAGGCC GGGCTGGTATACAATGCTCTTTTGGAGCGCGTGGCCCGTTACAACAGCAGCAACGTGCAGACCAACCTGGACCGGAT GGTGACCGATGTGCGCGAGGCTGTGTCTCAGCGCGAGCGGTTCCAGCGCGACGCCAACTTGGGGTCATTGGTAGCGC TAAACGCTTTCCTTAGCACCCAGCCCGCCAACGTGCCCCGTGGTCAGCAAGACTATACAAACTTTTTGAGTGCATTG AGACTCATGGTATCTGAGGTGCCCCAGAGCGAGGTGTACCAGTCCGGGCCAGATTACTTCTTCCAGACCAGCAGACA GGGCTTGCAGACAGTGAACCTGACCCAGGCTTTCAAGAACCTGAAGGGTCTGTGGGGAGTGCACGCCCCAGTAGGAG ATCGCGCGACCGTGTCTAGCTTGCTGACTCCCAACTCCCGCCTGCTGCTGCTGCTGGTATCCCCCTTCACTGACAGC GGTAGCATCGACCGCAACTCCTACTTGGGCTACCTGCTTAACCTGTATCGCGAGGCTATAGGACAGAGCCAGGTGGA CGAGCAGACCTATCAAGAAATCACCCAAGTGAGCCGCGCCCTGGGTCAGGAAGACACAGGCGGTTTGGAAGCCACCC TGAACTTCTTACTAACCAACCGGTCGCAGAAGATCCCTCCTCAGTATGCGCTTACCGCTGAGGAGGAGCGGATCCTA AGATACGTGCAACAGAGCGTTGGACTGTTTTTGATGCAGGAGGGGGCGACACCTACCGCCGCGCTGGATATGACAGC TCGAAACATGGAGCCCAGCATGTATGCTAGTAACAGGCCTTTCATTAACAAACTGCTGGACTACCTGCACAGGGCGG CCGCCATGAACTCTGATTATTTCACCAATGCTATTCTGAACCCACACTGGCTGCCCCCACCTGGTTTCTACACTGGC GAATACGACATGCCCGATCCCAATGACGGGTTCCTATGGGACGATGTGGACAGTAGCATATTTTCCCCGCCGCCAGG TTATACGGTTTGGAAGAAGGAAGGGGGCGATAGAAGGCACTCTTCCGTATCGTTGCCCGGAACGGCTGGTGCTGCCG CGGCCGTGCCCGAAGCTGCGAGTCCTTTCCCTAGCTTGTCCTTTTCACTAAACAGCGTTCGCAGCAGTGAACTGGGG AGAATAAACCGCCCGCGCTTGATGGGCGAGGATGAGTACTTGAATGACTCTTTGCTGAGGCCAGAGAGGGAAAAGAA CTTCCCTAACAATGGAATAGAGAGCCTGGTGGATAAGATGAGTAGATGGAAGACCTATGCGCAGGATCACAGAGACG AGCCCAGGATCTTGGGGGCTACAAGCAGACCGAGCCGTAGACGCCAGCGCCACGACAGGCAGATGGGTCTTGTGTGG GACGACGAGGACTCTGCCGATGACAGCAGCGTGTTGGACTTGGGTGGAAAAGGAGTTGGCAACCCGTTCGCTCATCT GCGTCCCCGTTTCGGTCGCATGTTGTAAAAGTGAAAGTAAAAATAAAAAGGCAACTCACCAAGGCCATGGCAACCGA GCGTGCGTTCGTTCTTTTTTTTGTTATCTGTATCTAGTACGATGAGGAGACGAGCCGTGCTAGGCGGAGCGGTGGTG TATCCGGAGGGTCCTCCTCCTTCTTACGAGAGCGTGATGCAGCAACAGGCGGCGATGATACAGCCCCCACTGGAGGC TCCCTTCGTACCCCCTCGGTACCTGGCGCCTACGGAAGGGAGAAATAGCATTCGTTACTCGGAGCTGTCACCCCAGT ACGATACCACCAAGTTGTATCTGGTGGACAACAAGTCGGCGGACATCGCCTCCCTGAACTATCAGAACGACCACAGC AACTTCCTGACCACAGTGGTGCAGAACAATGACTTTACCCCCACGGAGGCTAGCACCCAGACCATTAACTTTGACGA GCGGTCGCGGTGGGGCGGTCAGCTGAAGACCATTATGCACACCAACATGCCCAACGTGAACGAGTACATGTTCAGCA ACAAGTTTAAGGCGAGGGTGATGGTATCTAGGAAGGCTCCTGAAGGTGTTACAGTAAATGATCATAAAGATGATATT TTGAAATATGAGTGGTTTGAGTTCACTTTACCAGAAGGTAACTTCTCAGCTACCATGACCATCGACCTGATGAACAA TGCCATCATTGACAACTACCTGAAAATTGGCAGACAGAATGGAGTGCTGGAAAGTGACATTGGTGTTAAGTTTGACA CTAGAAACTTCAGGCTCGGGTGGGACCCCGAAACTAAGTTGATTATGCCAGGGGTCTACACTTATGAGGCATTCCAT CCTGACATTGTTTTGTTGCCTGGTTGCGGGGTAGATTTTACTGAAAGCCGACTTAGCAACTTGCTTGGCATCAGGAA GAGACATCCATTCCAGGAGGGTTTCAAAATCATGTATGAAGATCTTGAAGGGGGTAATATTCCTGCCCTTTTGGATG TCACTGCCTATGAGGAAAGCAAAAAGGATACCACTACTGAAACAGGCGAAAAGGCGGTGGTTAAAACAACCACAGTG GCTGTTGCAGAGGAAACCAGTGAAGATGATAATATAACTAGAGGAGATACTTATATAACTGAAAAACAAAAACGTGA AGCTGCAGCTGCAGAACTATTACTTATGTCTGAAGTTAAAAAAGAGTTAAAGATCCAACCTTTAGAAAAAGACAGCA AGAATAGAAGCTACAATGTCTTGGAAGACAAAATCAACACAGCCTACCGCAGCTGGTACCTGTCCTACAATTATGGT AACCCTGAGAAAGGAATAAGGTCCTGGACACTGCTCACCACTTCGGATGTCACCTGTGGAGCCGAGCAGGTCTACTG GTCGCTCCCCGACATGATGCAAGACCCCATCACCTTCCGCTCCTCGAGACAAGTCAACAACTACCCAGTAGTGGGTG CAGAGCTTATGCCGGTCTTCTCAAAGAGTTTCTACAATGAGCAAGCCGTGTACTCTCAGCAGCTCCGACAGTCCACC TCGCTCACGCACGTCTTCAACCGCTTCCCTGAGAACCAGATCCTCATCCGCCCGCCGGCGCCCACAATTACCACCAT CAGTGAAAACGTTCCTGCTCTCACAGATCACGGGACCCTGCCGTTACGCAGCAGTATCCGGGGAGTCCAGCGCGTGA CCGTTACTGACGCCAGACGTCGCACCTGTCCCTACGTTTACAAGGCCCTGGGCATAGTCGCGCCGCGCGTTCTTTCA AGCCGCACTTTCTAAAAAAAAAAAAAAATGTCCATTCTCATCTCGCCCAGTAATAATACCGGTTGGGGACTGCATGC GCCCACCAAGATGTACGGAGGCGCCCGCAAACGCTCTACCCAGCACCCCGTGCGCGTTCGCGGTCATTTCCGCGCTC CCTGGGGCGCCCTCAAGGGCCGTACCCGCACTCGGACCACGGTCGATGATGTGATCGACCAGGTGGTTGCCGATGCT CGTAATTATACTCCTACTGCGCCTACATCTACTGTGGATGCAGTTATTGACAGTGTAGTGGCAGACGCTCGCGCCTA TGCTCGCCGGAAGAGCCGAAGGAGGCGCATCGCCAGGCGCCACAGGGCTACTCCCGCTATGCGAGCTGCAAAAGCTA TTCTGCGGAGGGCCAAACGTGTGGGACGAAGAGCCATGCTTAGAGCGGCCAGACGCGCGGCTTCTGGTGCTAGCAGC GGCAGGTCCCGCAGGCGCGCGGCCACGGCGGCAGCAGCGGCCATTGCCAACATGGCCCAACCGCGAAGAGGCAATGT GTATTGGGTGCGCGATGCCACTACCGGCCAGCGCGTGCCCGTGCGCACCCGCCCCCCTCGCACTTAGAAGATACTGA GCAGTCTCCGATGTTGTGTCCCAGCGGCAAGTATGTCCAAGCGCAAATACAAGGAAGAGATGCTCCAGGTCATCGCG CCTGAAATCTACGGTCCGCCGATGAAGGATGAAAAAAAGCCCCGCAAAATCAAGCGGGTTAAAAAGGACAAAAAAGA AGAAGATGGCGATGATGGACTGGTGGAGTTTGTGCGCGAGTTCGCGCCAAGACGGCGCGTGCAGTGGCGCGGTCGAA AAGTACGCCAAGTGCTTAGACCCGGGACCACTGTGGTCTTTACACCTGGCGAGCGTTCCAGCACTACTTTTAAACGG TCCTATGATGAGGTGTATGGGGATGACGATATTATTGAGCAGGCGGCAGACCGCCTTGGCGAGTTTGCTTATGGCAA GCGCACAAGATCCAGTCCCAAAGAGGAGGCGGTATCTATTCCCTTGGATCATGGAAATCCCACCCCTAGCCTCAAAC CAGTCACCCTGCAGCAAGTGCTGCCCGTACCTGCGAGCAGAGGCGTAAAGCGCGAGGGTGAGGACCTATATCCCACC ATGCAGCTAATGGTGCCCAAGCGGCAGAGATTAGAAGACGTACTGGAGAAAATGAAAGTGGATGCCGATATCCAGCC TGAGGTGAAAGTGAGACCCATCAAGGAAGTGGCGCCAGGTTTGGGAGTACAAACCTTTGACATCAAGATTCCCACTG AGTCCATGGAAGTGCAGACCGAACCTGCAAAACCCACAGTCACCTCAATTGAGGTTCAGACGGAACCCTGGATGCCC GCGCCCGTTGCCGCCCCCAGCACCACTAGAAGATCACGTCGAAAGTATGGCCCAGCAAGTCTGCTAATGCCCAACTA TGCTCTGCACCCATCCATCATTCCCACTCCGGGTTACAGAGGCACTCGCTACTATCGAAGTCGGAGCAACACCTCAC GCCGCCGCAAACTACCTGCAAGTCGCACTCGCCGTCGCCGCCGCCGCACCACTGCCAGCAAATTAACTCCCGCCGCC CTGGTGCGGAGAGTGTACCGCGATGGTCGCGCTGAACCTCTGACGCTGCCGCGCGCGCGCTATCATCCAAGCATCAC CACTTAATGACTGTTGACGCTGCCTCCTTGCAGATATGGCTCTCACTTGCCGCCTTCGCGTCCCCATTACTGGCTAC CGAGGAAGAAACTCGCGCCGTAGAAGGATGTTGGGGCGAGGGATGCGCCGCCACAGACGAAGGCGCGCTATCAGCAA GCGATTAGGGGGTGGCTTTCTGCCAGCTCTTATACCCATCATCGCCGCGGCGATCGGGGCGATACCAGGCATAGCTT CCGTGGCGGTTCAGGCCTCGCAGCGCCACTAACAATGGAAAAATTTATAAATAAAAAATAGAATGGACTCTGACGCT CCTGGTCCTGTGACTATGTTTTTGTAGAGATGGAAGACATCAATTTTTCATCCCTGGCTCCGCGACACGGCACGAGG CCGTACATGGGCACCTGGAGCGACATCGGCACGAGCCAACTGAACGGGGGCGCCTTCAATTGGAGCAGTATCTGGAG CGGGCTTAAAAATTTTGGCTCGACCATAAAAACCTATGGGAACAAAGCTTGGAACAGCAGCACAGGGCAGGCCCTTA GAAATAAGCTTAAGGAGCAGAACTTCCAACAAAAGGTGGTCGATGGTATCGCCTCTGGTATTAACGGCGTAGTGGAT CTAGCCAACCAGGCTGTGCAGAAACAGATAAACAGCCGCCTGGACCCGCCGCCCGCAACTCCTGGTGAAATGGAAGT GGAGGAAGAGCTTCCTCCGCTGGAGAAGCGGGGCGACAAGCGACCGCGTCCCGAGCTGGAACAGACGTTGGTGACGC GCGCAGACGAGCCCCCTTCATACGAGGAGGCAGTAAAGCTCGGAATGCCCACTACCAGGCCTGTAGCTCACATGGCT ACCGGGGTGATGAAACCTTCTCAGTCGCATCGGCCTGCCACCTTGGACTTGCCTCCTCCCCCTGCTTCTGCGGCGCC TATTCCCAAACCTGTCGCTACCAGAAAGCCCACCGCCGTACAGCCCGTCGCCGTAGCCAGACCGCGTCCTGGGGCAC ACCGCGCCCGAAAGCAAACTGGCAGAGTACTCTGAACAGCATCGTGGGTCTGGGCGTGCAGAGTGTAAAGCGCCGTC GCTGCTATTAATTAAATATGGAGTAGCGCTTAACTTGCTTGTCTGTGTGTATGTATCATCACCATGCCGCCGCAGCA GAGGAGAAAGGAAGAGGTCGCGCGCCGAGGCTGAGTTGCTTTCAAGATGGCCACCCCATCGATGATGCCCCAATGGG CATACATGCACATCGCCGGACAGGATGCTTCGGAGTACCTGAGTCCGGGTCTGGTGCAGTTCGCCCGTGCAACAGAC ACCTACTTCAGTATGGGGAACAAGTTTAGAAACCCCACAGTGGCGCCCACCCACGATGTGACCACCGACCGTAGCCA GCGACTAATGCTGCGCTTCGTGCCCGTTGACCGGGAAGACAATACCTACTCTTACAAAGTTCGCTACACGCTGGCTG TAGGGGACAACAGAGTACTGGATATGGCCAGCACGTTCTTTGACATCCGCGGCGTGCTGGACCGGGGCCCTAGCTTC AAACCCTACTCCGGGACCGCCTACAACAGCCTGGCTCCCAAGGGAGCGCCCAACACCTGCCAGTGGAAGGATTCTGA CAGCAAAATGCATACCTTTGGGGTAGCTGCCATGCCCGGTGTTACTGGGAAAAAGATAGAAGCTGATGGGCTGCCTA TTGGAATAGATTCAACTTCTGGAACTGACACAGTAATTTATGCTGATAAAACTTTCCAACCAGAACCACAAGTTGGA AATGCCAGTTGGGTTGACGCCAATGGTACAGAGGAAAAATATGGAGGCAGAGCTCTGAAGGACACTACAAAGATGAA ACCCTGCTATGGTTCTTTCGCCAAGCCTACCAACAAAGAAGGTGGTCAGGCTAACTTAAAAGATTCAGAAACCGCCG CCACCACTCCTAACTATGATATAGATCTGGCTTTCTTTGACAACAAAAATATTGCTGCTAACTACGATCCAGATATT GTAATGTACACAGAAAATGTTGACTTGCAGACTCCAGATACTCATATTGTATACAAACCTGGAACAGAGGACACCAG CTCTGAATCCAATTTGGGTCAGCAAGCCATGCCTAACAGACCCAACTACATTGGCTTCAGAGACAATTTTATTGGGC TCATGTACTACAACAGCACTGGCAATATGGGGGTGCTGGCCGGTCAGGCCTCTCAGCTGAATGCTGTGGTTGACTTG CAAGACAGAAACACTGAACTGTCCTACCAGCTCTTGCTTGACTCTCTGGGTGACAGAACCCGGTATTTCAGTATGTG GAATCAGGCGGTGGACAGCTATGATCCTGATGTGCGTATTATTGAAAACCATGGTGTGGAGGACGAATTGCCAAACT ATTGCTTTCCGTTGAATGGTGTGGGATTTACAGACACTTACCAAGGTGTTAAAGTTAAAACAGATGCAGTTGCTGGA ACCAGTGGAACACAGTGGGACAAAGATGACACCACAGTTAGTACTGCTAATGAAATCCATGGAGGCAATCCTTTTGC CATGGAAATCAACATCCAAGCCAATCTATGGCGAAGTTTCCTTTATTCCAATGTGGCTTTGTATCTCCCAGACTCGT ATAAATACACCCCGTCCAATGTCACTCTCCCAGAAAACAAAAACACCTACGACTACATGAACGGGCGGGTGGTGCCG CCATCTCTAGTAGACACCTATGTGAACATTGGTGCCAGGTGGTCTCTGGATGCCATGGACAATGTCAACCCATTCAA CCACCACCGTAACGCTGGCTTGCGTTACCGATCCATGCTTCTGGGTAACGGACGTTATGTGCCTTTCCACATACAAG TGCCTCAAAAATTCTTCGCTGTTAAAAACCTGCTGCTTCTCCCAGGCTCCTACACTTATGAGTGGAACTTTAGGAAG GATGTAAACATGGTTCTACAGAGTTCCCTTGGTAACGACCTACGGGTAGATGGCGCCAGCATCAGTTTCACGAGCAT CAATCTTTATGCTACTTTTTTCCCCATGGCTCACAACACCGCTTCCACCCTTGAAGCCATGCTGCGGAATGACACCA ATGATCAGTCATTCAACGACTACCTATCTGCAGCTAACATGCTCTACCCCATACCTGCCAACGCAACCAATATTCCC ATTTCCATTCCTTCTCGCAACTGGGCGGCTTTCAGAGGCTGGTCATTTACCAGACTGAAAACCAAAGAAACTCCCTC TTTGGGGTCTGGATTTGACCCATACTTTGTCTATTCCGGTTCTATTCCCTACCTGGATGGTACCTTCTATCTAAATC ACACTTTTAAGAAGGTTTCCATCATGTTTGACTCTTCAGTGAGCTGGCCTGGAAATGACAGGTTACTATCTCCTAAC GAATTTGAAATAAAGCGCACTGTGGATGGCGAAGGCTACAACGTAGCCCAATGCAACATGACCAAAGACTGGTTCTT GGTACAGATGCTCGCCAACTACAACATTGGCTACCAGGGCTTTTACATCCCTGAGGGATACAAGGATCGCATGTACT CCTTTTTCAGAAACTTCCAGCCTATGAGCAGGCAGGTGGTTGATGAGGTTAATTACACTGACTACAAAGCCGTCACC TTACCATATCAACACAACAACTCTGGCTTTGTAGGATACCTTGCGCCTACTATGAGACAAGGGGAACCTTACCCAGC CAATTATCCATACCCGCTCATCGGAACTACTGCCGTTAAGAGTGTTACCCAAAAAAAGTTCCTGTGCGACAGGACCA TGTGGCGCATACCGTTCTCCAGCAACTTCATGTCCATGGGGGCCCTTACAGACCTGGGACAAAACCTGCTCTATGCC AACTCGGCCCATGCACTGGACATGACTTTTGAGGTGGATCCCATGGATGAGCCCACCCTGCTTTATCTTCTTTTCGA AGTCTTCGACGTGGTCAGAGTGCACCAGCCACACCGCGGCGTCATCGAGGCCGTCTACCTGCGCACACCGTTCTCGG CCGGCAACGCCACCACATAAGAAGCCTCTTGCTTCTTGCAAGCAGCAGCTGCAGCCATGTCATGCGGGTCCGGAAAC GGCTCCAGCGAGCAAGAGCTCAAAGCCATCGTCCGAGACCTGGGTTGCGGTCCCTATTTCCTGGGAACCTTTGACAA GCGTTTCCCGGGGTTCATGGCCCCCGACAAGCTCGCCTGCGCCATAGTCAACACTGCCGGACGCGAGACGGGGGGAG AGCACTGGCTGGCTTTTGGTTGGAACCCGCGCTCCAACACCTGCTACCTTTTTGATCCTTTTGGGTTCTCGGATGAG CGACTCAAACAGATTTACCAGTTTGAGTACGAGGGGCTCCTGCGCCGCAGTGCCCTTGCTACCAAAGACCGCTGCAT CACCCTGGAAAAGTCCACCCAGAGCGTGCAGGGCCCACGCTCAGCCGCCTGTGGACTTTTTTGCTGTATGTTCCTTC ATGCCTTTGTGCACTGGCCCGACCGTCCCATGAACGGAAACCCCACCATGAAGTTGCTGACTGGGGTGCCCAACAGC ATGCTCCAATCTCCCCAAGTCCAGCCCACCCTGCGCCGCAACCAGGAGGCACTATACCGCTTCCTAAACACCCACTC ATCTTACTTTCGTTCTCACCGCGCACGCATCGAAAGGGCCACCGCGTTTGACCGTATGGATATGCAATAAGTCATGT AAAACCGTGTTCAATAAAAAGCACTTTATTTTTACATGCACTAAGGCTCTGGTTTTTTGCTCATTCGTTTTCATCAT TCACTCAGAAATCAAATGGGTTCTGGCGGGAGTCATAGTGGCCCGCGGGCAGGGATACGTTGCGGAACTGTAACCTG TTCTGCCACTTGAACTCGGGGATCACCAGCTTGGGAACTGGAATCTCGGGAAAGGTGTCTTGCCACAACTTTCTGGT CAGTTGCAGGGCGCCAAGCAGGTCAGGAGCAGAGATCTTGAAATCACAGTTGGGGCCGGCATTCTGGACACGGGAGT TGCGGTACACTGGGTTGCAACACTGGAACACCATCAAGGCTGGGTGTCTCACGCTTGCCAGCACGGTCGGGTCACTG ATGGTAGTCACATCCAAGTCTTCAGCATTGGCCATCCCAAAGGGGGTCATCTTACAGGTCTGCCTGCCCATCACGGG AGCGCAGCCTGGCTTGTGGTTGCAATCGCAATGAATGGGAATCAGCATCATCCTGGCTTGGTCGGGGGTTATCCCTG GATATACGGCCTTCATGAAGGCTTCGTACTGCTTGAAAGCTTCCTGAGCCTTACTTCCCTCGGTGTAGAACATTCCA CAGGACTTGCTGGAAAATTGGTTAGTAGCACAGTTGGCATCATTTACACAGCAGCGGGCATCGTTGTTGGCCAACTG AACCACATTTCTGCCCCAGCGGTTCTGGGTGATCTTGGCTCTGTCTGGGTTCTCCTTCATAGCGCGCTGCCCGTTCT CGCTCGCCACATCCATCTCGATAATGTGGTCCTTCTGGATCATGATAGTGCCATGCAGGCATTTCACCTTGCCTTCG TAATCGGTGCATCCATGAGCCCACAGAGCGCACCCGGTGCACTCCCAATTATTGTGGGCGATCTCAGAATAAGAATG CACCAATCCCTGCATGAATCTTCCCATCATCGCTGTCAGGGTCTTCATGCTACTAAATGTCAGCGGAATGCCACGGT GCTCCTCGTTCACATACTGGTGGCAGATACGCTTGTACTGCTCGTGCTGCTCTGGCATCAGCTTGAAAGAGGTTCTC AGGTCATTATCCAGCCTATACCTCTCCATTAGCACAGCCATCACTTCCATGCCCTTCTCCCAGGCAGATACCAGGGG CAAGCTCAAAGGATTCCTAACAGCAATAGAAGTAGCTCCTTTAGCTATAGGGTCATTCTTGTCGATCTTCTCAACAC TTCTCTTGCCATCCTTCTCAATGATGCGCACCGGGGGGTAGCTGAAGCCCACGGCCACCAACTGAGCCTGTTCTCTT TCTTCTTCGCTGTCCTGGCTGATGTCTTGCAGAGGGACATGCTTGGTCTTCCTGGGCTTCTTCTTGGGAGGGATCGG GGGAGGACTGTTGCTCCGTTCCGGAGACAGGGATGACCGCGAAGTTTCGCTTACCAGTACCACCTGGCTCTCGATAG AAGAATCGGACCCCACGCGACGGTAGGTGTTCCTCTTCGGGGGCAGAGGTGGAGGCGACTGAGATGGGCTGCGGTCT GGCCTTGGAGGCGGATGGCTGGCAGAGCCCATTCCGCGTTCGGGGGTGTGCTCCCGTTGGCGGTCGCTTGACTGATT TCCTCCGCGGCTGGCCATTGTGTTCTCCTAGGCAGAGAAACAACAGACATGGAAACTCAGCCATCACTGCCAACATC GCTGCAAGCGCCATCACACCTCGCCCCCAGCAGCGACGAGGAGGAGAGCTTAACCACCCCACCACCCAGTCCCGCTA CCACCACCTCTACCCTCGATGATGAGGAGGAGGTCGACGCAGCCCAGGAGATGCAGGCGCAGGATAATGTGAAAGCG GAAGAGATTGAGGCAGATGTCGAGCAGGACCCGGGCTATGTGACACCGGCGGAGCACGAGGAGGAGCTGAAACGTTT TCTAGACAGAGAGGATGACGACCGCCCAGAGCATCAAGCAGATGGCGATCACCAGGAGGCTGGCCTCGGGGATCATG TTGCCGACTACCTCTCCGGGCGTGGGGGGGAGGACGTGCTCCTCAAACATCTAGCAAGGCAGTCGCTCATAGTTAAA GACGCACTACTCAACCTCACCGAAGTGCCCATCAGTGTGGAAGAGCTTAGCCGCGCCTACGAGCTGAACCTCTTTTC GCCTCAGATACCCCCCAAGCGGCAGCCAAACGGCACCTGCGAGGCCAACCCTCGACTCAACTTCTATCCAGCTTTTA CTGTCCCCGAAGTGCTGGCCACCTACCACATCTTTTTTAAGAACCAAAAGATTCCAGTCTCCTGCCGCGCCAACCGC ACCCGCGCAGATGCCCTTCTCAACTTGGGTCCGGGAGCTCGCTTACCTGATATAGCTTCCTTGGAAGAGGTTCCAAA GATCTTTGAGGGTCTGGGAAGTGATGAGACTCGGGCCGCAAATGCTCTGCAACAGGGAGAGAATGGCATGGATGAAC ATCACAGCGCTCTAGTGGAACTGGAGGGTGACAATGCCCGGCTTGCAGTGCTCAAGCGCAGTATCGTGGTCACCCAT TTTGCCTACCCCGCTGTTAACCTGCCGCCCAAAGTCATGAGCGCTGTCATGGACCATCTGCTCATCAAACGAGCAAG TCCACTTTCAGAAAACCAGAACATGCAGGATCCAGACGCCTCGGACGAGGGCAAGCCGGTAGTCAGTGACGAGCAGC TATCTCGCTGGCTGGGTACCAACTCCCCCCGAGATTTGGAAGAAAGACGCAAGCTTATGATGGCTGTAGTGCTAGTA ACTGTTGAGTTGGAGTGTCTGCGCCGCTTTTTTACCGACCCCGAGACCCTGCGCAAGCTAGAGGAGAACCTGCACTA CACCTTCAGACATGGCTTCGTGCGGCAGGCATGCAAGATCTCCAACGTGGAGCTCACCAACCTGGTTTCATACATGG GCATTTTGCATGAGAACCGGCTAGGGCAGAGCGTTCTGCACACCACCCTGAAGGGGGAGGCCCGCCGCGACTACATC CGAGACTGTGTCTACCTCTACCTCTGCCATACCTGGCAGACTGGTATGGGTGTGTGGCAACAGTGTTTGGAAGAGCA GAACCTTAAAGAGCTGGACAAGCTCTTGCAGAGATCCCTCAAAGCCCTGTGGACAGGTTTTGACGAGCGCACCGTCG CCTCGGACTTGGCGGACATCATCTTCCCCGAGCGTCTTACGGTTACTCTGCGAAACGGCCTGCCAGACTTCATGAGC CAGAGCATGCTTAACAACTTTCGCTCTTTCATCCTGGAACGCTCCGGTATCCTGCCTGCCACCTGCTGTGCGCTGCC CTCCGACTTTGTGCCTCTCACCTACCGCGAGTGCCCACCGCCGCTATGGAGCCACTGCTACCTATTCCGCCTGGCCA ACTACCTCTCCTACCACTCGGATGTGATAGAGGATGTGAGCGGAGACGGCCTGCTGGAATGCCACTGCCGATGCAAT TTATGCACACCCCACCGCTCCCTCGCCTGCAACCCCCAGTTGCTAAGCGAGACCCAGATCATCGGCACCTTCGAGTT GCAGGGTCCCAACAGTGAAGGCGAGGGGTCTTCTCCGGGGCAGAGTCTGAAACTGACACCGGGGCTGTGGACCTCCG CCTACCTGCGCAAGTTTCATCCCGAGGACTATCATCCCTATGAGATCAGGTTCTATGAGGACCAGTCACATCCTCCC AAAGTCGAGCTCTCAGCCTGCGTCATCACCCAGGGGGCAATTCTGGCCCAATTGCAAGCCATCCAAAAATCCCGCCA AGAATTTCTGCTGAAAAAGGGAAGCGGGGTCTACCTTGACCCCCAGACCGGTGAGGAGCTCAACACAAGGTTCCCCC AGGATGTCCCATCGCCGAGGAAGCAAGAAGCTGAAGGTGCAGCTGTCACCCCCAGAGGATATGGAGGAAGACTGGGA CAGTCAGGCAGAGGAGGAGATGGAAGATTGGGACAGCCAGGCAGAGGAGGTGGACAGCCTGGAGGAAGACAGTTTGG AGGAGGAAGACGAGGAGGCAGAGGAGGTGGAAGAAGCAACCGCCGCCAAACAGTTGTCATCGGCGGCGGAGACAAGC AAGTCCCCAGACAGCAGCACGGCTACCATCTCCGCTCCGGGTCGGGGGGCCCAGCGGCGGCCCAACAGTAGATGGGA CGAGACCGGGCGATTCCCAAACCCGACCACCGCTTCCAAGACCGGTAAGAAGGAGCGACAGGGATACAAGTCCTGGC GTGGACATAAAAACGCTATCATCTCCTGCTTGCATGAATGCGGGGGCAACATATCCTTCACCCGGCGATACCTGCTT TTCCACCACGGTGTGAACTTCCCCCGCAATATCTTGCATTACTACCGTCACCTCCACAGCCCCTACTGCAGTCAGCA AGTCCCGGCAACCCCGACAGAAAAAGACAGCAGCGACAACGGTGACCAGAAAACCAGCAGTTAGAAAATCCACAACA AGTGCAGCAGGAGGAGGACTGAGGATCACAGCGAACGAGCCAGCGCAGACCAGAGAGCTGAGGAACCGGATCTTTCC AACCCTCTATGCCATCTTCCAGCAGAGTCGGGGGCAAGAGCAGGAATTAAAAGTAAAAAACCGATCTCTGCGCTCGC TCACCAGAAGTTGTTTGTATCACAAGAGCGAAGACCAACTTCAGCGCACTCTCGAGGACGCCGAGGCTCTCTTCAAC AAGTACTGCGCGCTGACTCTTAAAGAGTAGCCCTTGCCCGCGCTCATTCGAAAACGGCGGGAATCACGTCACCCTTG GCAGCTGTCCTTTGCCCTCGTCATGAGTAAAGAGATTCCCACGCCTTACATGTGGAGCTATCAGCCCCAAATGGGGT TGGCAGCAGGTGCTTCCCAGGACTACTCCACCCGCATGAATTGGCTTAGCGCCGGGCCCTCAATGATATCACGGGTT AATGATATACGAGCTTATCGAAACCAGTTACTCCTAGAACAGTCAGCTCTCACCACCACACCCCGCCAACACCTTAA TCCCCGAAATTGGCCCGCCGCCCTGGTGTACCAGGAAAATCCCGCTCCCACCACCGTACTACTTCCTCGAGACGCCC AGGCCGAAGTTCAGATGACTAACGCAGGTGTACAGCTGGGGGGGGTTCCGCCCTATGTCGTCACCGACCTCAACAG AGTATAAAACGCCTGGTGATCAGAGGCCGAGGTATCCAGCTCAACGACGAGTCGGTTAGCTCTTCGCTTGGTCTGCG ACCAGACGGAGTCTTCCAGATCGCCGGCTGTGGGAGATCTTCCTTCACTCCTCGTCAGGCTGTGCTGACTTTGGAGA GTTCGTCCTCGCAGCCCCGCTCGGGCGGCATCGGAACTCTCCAGTTTGTGGAGGAGTTTACTCCCTCTGTCTACTTC AACCCCTTCTCCGGCTCTCCTGGCCAGTACCCGGACGAGTTCATACCGAACTTCGACGCAATCAGCGAGTCAGTGGA TGGCTATGATTGATGTCTAATGGTGGCGCGGCTGAGCTAGCTCGACTGCGACACCTAGACCACTGCCGCCGCTTTCG CTGTTTCGCCCGGGAACTCACCGAGTTCATCTACTTCGAACTCTCCGAGGAGCACCCTCAGGGTCCGGCCCACGGAG TGCGGATTACCATCGAAGGGGGAATAGACTCTCGCCTGCATCGCATCTTCTCCCAGCGGCCCGTGCTGATTGAGCGC GACCAGGGAAATACAACCATCTCCATCTACTGCATTTGTAACCACCCCGGATTGCATGAAAGCCTTTGCTGTCTTGT TTGTGCTGAGTTTAATAAAAACTGAGTTAAGACCCTCCTACGGACTACCGCTTCTTCAATCAGGACTTTACAACACC AACCAGATCTTCCAGAAGACCCAGACCCTTCCTCCTCTGATCCAGGACTCTAACTCTACCTTACCAGCACCCTCCAC TACTAACCTTCCCGAAACTAACAAGCTTGGATCTCATCTGCAACACCGCCTTTCACGAAGCCTTCTTTCTGCCAATA CTACCACTCCCAAAACCGGAGGTGAGCTCCGCGGTCTTCCTACTGACGACCCCTGGGTGGTAGCGGGTTTTGTAACG TTAGGAGTAGTTGCGGGTGGGCTTGTGCTAATCCTTTGCTACCTATACACACCTTGCTGTGCATATTTAGTCATATT GTGCTGTTGGTTTAAGAAATGGGGGCCATACTAGTCGTGCTTGCTTTACTTTCGCTTTTGGGTCTGGGCTCTGCTAA TCTCAATCCTCTTGATCACGATCCATGTCTAGACTTCGACCCAGAAAACTGCACACTTACTTTTGCACCCGACACAA GCCGTCTCTGTGGAGTTCTTATTAAGTGCGGATGGGACTGCAGGTCCGTTGAAATTACACATAATAACAAAACATGG AACAATACCTTATCCACCACATGGGAGCCAGGAGTTCCCGAGTGGTATACTGTCTCTGTCCGAGGTCCTGACGGTTC CATTCGCATTAGTAACAACACTTTCATTTTTTCTGAAATGTGCGATCTGGCCATGTTTATGAGCAAACAGTATGACC TATGGCCTCCTAGCAAAGAGAACATTGTGGCATTTTCCATTGCTTATTGCTTGGTAACATGCATCATCACTGCTATC ATTTGTGTGTGCATACACTTGCTTATAGTTATTCGCCCTAGACAAAGCAATGAGGAAAAAGAGAAAATGCCTTAACC TTTTTCCTCATACCTTTTCTTTACAGCATGGCTTCTGTTACAGCTCTAATTATTGCCAGCATTGTCACTGTCGCTCA CGGGCAAACAATTGTCCATATTACCTTAGGACATAATCACACTCTTGTAGGGCCCCCAATTACTTCAGAGGTTATTT GGACCAAACTTGGAAGTGTTGATTATTTTGATATAATTTGCAACAAAACTAAACCAATATTTGTAATCTGCAACAGA CAAAATCTCACGTTAATTAATGTCAGCAAAATTTATAACGGTTACTATTATGGTTATGATAGATCCAGTAGTCAATA TAAAAATTACTTAGTTCGCATAACTCAACCCAAATCAACAGTGCCAACTATGACAATAATTAAAATGGCTAATAAAG CATTAGAAAATTTTACATTACCAACAACGCCCAATGAAAAAAACATTCCAAATTCAATGATTGCAATTATTGCGGCG GTGGCATTGGGAATGGCACTAATAATAATATGCATGTTCCTATATGCTTGTTGCTATAAAAAGTTTAAACATAAACA GGATCCACTACTAAATTTTAACATTTAATTTTTTATACAGATGATTTCCACTACAATTTTTATCATTACTAGCCTTG CAGCTGTAACTTATGGCCGTTCACACCTAACTGTACCTGTTGGCTCAACATGTACACTACAAGGACCCCAAGAAGGC CATGTCACTTGGTGGAGAATATATGATAATGGAGGGTTCGCTAGACCATGTGATCAGCCTGGTACAAAATTTTCATG CAACGGAAGAGACTTGACCATTATTAACATAACATTAAATGAGCAAGGCTTCTATTATGGAACCAACTATAAAAATA GTTTAGATTACAACATTATTGTAGTGCCAGCCACCACTTCTGCTCCCCGCAAATCCACTTTCTCTAGCAGCAGTGCC AAAGCAAGCACAATTCCTAAAACAGCTTCTGCTATGTTAAAGCTTCGAAAAATCGCTTTAAGTAATTCCACAGCCGC TCCCAATACAATTCCTAAATCAACAATTGGCATCATTACTGCCGTGGTAGTGGGATTAATAATTATATTTTTGTGCA TAATGTACTACGCCTGCTGCTATAGAAAACATGAACAAAAAGGTGATGCATTACTAAATTTTGATATTTAATTTTTT ATAGAATTATGATATTGTTTCAATCAAATACCACTAACACTATCAATGTGCAGACTACTTTAAATCATGACATGGAA AACCACACTACCTCCTATGCATACACAAACATTCAGCCTAAATACGCTATGCAACTAAGAAATCACCATACTAATTG TAATTGGAATTCTTATACTATCTGTTATTCTTTATTTTATATTCTGCCGTCAAATACCCAATGTTCATAGAAATTCT AAAAGACGACCCATCTATTCTCCTATGATTAGTCGTCCCCATATGGCTCTGAATGAAATCTAAGATCTTTTTTTTTC TCTTACAGTATGGTGAACATCAATCATGATTCCTAGAAATTTCTTCTTCACCATACTCATCTGTGCTTTCAATGTCT GTGCTACTTTCACAGCAGTAGCCACTGCAAGCCCAGACTGTATAGGACCATTTGCTTCCTATGCACTTTTTGCCTTC GTTACTTGCATCTGCGTGTGTAGCATAGTCTGCCTGGTTATTAATTTTTTCCAACTGGTAGACTGGATCTTTGTGCG AATTGCCTACCTACGTCACCATCCCGAATACCGCAATCAAAATGTTGCGGCACTTTTTAGGCTTATTTAAAACCATG CAGGCTATGCTGCCAGTCATTTTAATTCTGCTCCTACCCTGCATTGCCCTAGCTTCCACCGCCACTCGCGCTACACC TGAACAACTTAGAAAATGCAAATTTCAACAACCATGGTCATTTCTTGATTGCTACCATGAAAAATCTGATTTTCCCA CATACTGGATAGTGATTGTTGGAATAATTAACATACTTTCATGTACCTTTTTCTCAATCACAATATACCCCACATTT AATTTTGGGTGGAATTCTCCCAATGCACTGGGTTACCCACAAGAACCACATGAACATATCCCACTACAACACATACA ACAACCACTAGCACTGGTAGAGTATGAAAATGAGCCACAACCTTCACTGCCTCCTGCCATTAGTTACTTCAACCTAA CCGGCGGAGATGACTGAAATACTCACCACCTCCAATTCCGCCGAGGATCTGCTTGATATGGACGGCCGCGCCTCAGA ACAGCGACTCGCCCAACTACGCATACGCCAGCAGCAGGAACGTGCCGCCAAGGAGCTCAGGGATGCTATTGAAATTC ACCAATGCAAAAAAGGCATATTTTGTTTGGTAAAACAAGCCAAGATATCCTACGAGATTACCAATACTGACCATCGC CTCTCATACGAGCTCGGACCGCAGCGACAAAAATTCACTTGTATGGTGGGAATCAACCCCATAATCATCACCCAGCA AGCTGGAGATACCAAGGGTTGCATCCACTGTTCCTGCAGTTCCGCCGAGTGCATCTACACCCTGCTGAAGACCCTCT GCGGCCTTCGAGACCTCCTACCCATGAACTAATCAACCCAGCCCCTCACTTACCAATTACATAAAGCCAATTAATAA AAACACTTACTTGAAATCAGAAATAAGGTTTCTGTCTACGTTGTTTCCAAGCAGCACCTCACTTCCTTCTTCCCAAC TCTGGTACTCTAAGCCTCGGCGGGTGGCATACTTCCTCCACACTTTGAAAGGGATGTCAAATTTTAGTTCCTCTTCT TTGCCCACAATCTTCATTTCTTTATCCCCAGATGGCCAAACGAGCTCGGCTAAGCAGCTCCTTCAATCCGGTCTACC CCTATGAAGATGAAAGCAGCTCACAACACCCCTTTATAAACCCTGGTTTCATTTCCTCAAATGGTTTTGCACAAAGC CCAGATGGAGTTCTAACTCTTAAATGTGTTAATCCACTCACTACCGCCAGCGGACCCCTCCAACTTAAAGTTGGAAG CAGTCTTACAGTAGATACTATCGATGGGTCTTTGGAGGAAAATATAACTGCCGCAGCGCCACTCACTAAAACTAACC ACTCCATAGGTTTATTAATAGGATCTGGCTTGCAAACAAAGGATGATAAACTTTGTTTATCGCTGGGAGATGGGTTG GTAACAAAGGATGATAAACTATGTTTATCGCTGGGAGATGGGTTAATAACAAAAAATGATGTACTATGTGCCAAACT AGGACATGGCCTTGTGTTTGACTCTTCCAATGCTATCACCATAGAAAACAACACCTTGTGGACAGGCGCAAAACCAA GCGCCAACTGTGTAATTAAAGAGGGAGAAGATTCCCCAGACTGTAAGCTCACTTTAGTTCTAGTGAAGAATGGAGGA CTGATAAATGGATACATAACATTAATGGGAGCCTCAGAATATACTAACACCTTGTTTAAAAACAATCAAGTTACAAT CGATGTAAACCTCGCATTTGATAATACTGGCCAAATTATTACTTACCTATCATCCCTTAAAAGTAACCTGAACTTTA AAGACAACCAAAACATGGCTACTGGAACCATAACCAGTGCCAAAGGCTTCATGCCCAGCACCACCGCCTATCCATTT ATAACATACGCCACTGAGACCCTAAATGAAGATTACATTTATGGAGAGTGTTACTACAAATCTACCAATGGAACTCT CTTTCCACTAAAAGTTACTGTCACACTAAACAGACGTATGTTAGCTTCTGGAATGGCCTATGCTATGAATTTTTCAT GGTCTCTAAATGCAGAGGAAGCCCCGGAAACTACCGAAGTCACTCTCATTACCTCCCCCTTCTTTTTTTCTTATATC AGAGAAGATGACTGACAACAAAAAAAATAAAGATCAACTTTTTTATTGAAAATCAGTTTACAAGATTCGAGTAGTTA TTTTGCCCCCCTCTTCCCATTTTATAGAATACACAATTCTCTCCCCACGCACAGCTTTGAACATTTGAATTCCATTA GAGATAGACATAGTTTTAGATTCCACATTCCACACAGTTTCAGAGCGGGCCAATCTTGGATCAGTGATAGATATAAA TCCATCGGAACAGTCTTTCAAGGTGGTTTCACAGTCCAACTGCTGCGGCTGCGGCTCCGGGGTTTGGATTAGGGTCA TCTGGAAGAAGAACGATGGGAGTCATAATCCGAGAACGGGATCGGGCGGTTGTGTCTTAAACCTCGAAGCAATCGCT GTCTGCGCCGCTCCGTGCGACTGCTGCTGATGGGATCAGGATCCACAGTCTCTCGAAGCATAATTTTAATAGCCCTC AACATTAACATCCTGGTGCGATGGGCACAACAACGCATTCTAATTTCGCTTAGCTCACTGCAGTAGGTACAACACAT TACCACAATGTTGTTTAACAGGCCATAATTAAAGGTGCTCCAGCCAAAACTCATCTCAGGGATAATCATACCCGCGT GACCATCGTACCAAATCTTAATGTAAATTAGATGACGCCCCCTCCAGAACACACTGCCCACATACATAATTTCCTTG GGCATATGCATGTTCACAATTTCTCTGTACCATGGACAGCGCTGGTTAATCATACAGCCCCTAATAACCTTCCGGAA CCACATAGCTAGCACTGCTCCCCCAGCAATACATTGAAGAGAACCCGGCTGTTTACAGTGACAATGGAGAACCCACT TCTCTCGCCCATGGATCACTTGAGAATTAAATATATCTATAGTGGCACAACACAAACATAAATGCATGCATCTTTTC ATAACCCTCAACTCTTCGGGGGTTAAAAACATATCCCAGGGAATAGGAAGCTCTTGCAAAACAGTAAAGCTGGCAGA ACAAGGAAGACCACGAACACAACTTACACTATGCATAGTCATAGTATCACAATCTGGCAACAGCGGGTGGTCTTCAG TCATAGAAGCTCGGGTTTCATTTTCCTCACATCGTGGTAACTGGGCTCTGGTGTAAGGGTGATGTCTGGCGCATGAT GTCGAGCGTGCGCGCAACCTTGTCATAATGGAGTTGCTTCCTGACATTCTCGTATTTTGTATAGCAAAATGCGGCCC TGGCACAACACACTTTTCTTCGTCTTCTATCCTGCCGCTTAGTGTGTTCCGTCTGATAATTCAAGTACAGCCACACT CTTAAGTTGGTCAAAAGAATGCTGGCTTCAGTTGTAATCAAAACTCCATCATATTTAATTGTTCTAAGGAAATCATC CACGGTAGCATATGCAAATCCCAACCAAGCAATGCAACTGGATTGCGTTTCAAGCAGCAGAGGAGAGGGAAGAGACG GAAGAATCATGTTAATTTTTATTCCAAACGATCTCGCAGTACTTCAAATTGTAGATCGCGCAGATGGCATCTATCGC CCCCACTGTGTTGGTGAAAAAGCACAGCTAAATCAAAAGAAATGCGATTTTCAAGGTGCTCAACGGTGGCTTCCAAC AAAGCCTCCACGCGCACATCCAAAAACAAAAGAATACCAAAAGAAGGAGCATTTTCTAACTCCTCAAACATCATATT ACATTCCTGCACCATTCCCAGATAATTTTCAGCTTTCCAGCCTTGAATTATTCGTGTCAGTTCTTGTGGTAAATCCA AACCACACATTACAAACAGGTCCCGGAGGGCGCCCTCCACCACCATTCTTAAACACACCCTCATAATGACAAAATAT CTTGCTCCTGTGTCACCTGTAGCAAATTAAGAATGGCATCATCAATTGACATGCCCTTGGCTCTAAGTTCTTCTCTA AGTTCTAGTTGTAAATACTCTCTCATATTATCACCAAACTGCTTAGCCAGAAGCCCCCCGGGAACAATAGCAGGGGA CGCTACAGTGCAGTACAAGCGCAGACCTCCCCAATTGGCTCCAGCAAAAACAAGATTAGAATAAGCATACTGGGAAC CACCAGTAATATCATCAAAGTTGCTGGAAATATAATCAGGCAGAGTTTCTTGTAAAAATTGAATAAAAGAAAAATTT TCCAAAGAAACATTCAAAACCTCTGGGATGCAAATGCAATAGGTTACCGCGCTGCGCTCCAACATTGTTAGTTTTGA ATTAGTCTGTAAAATAAAAGAAACAAGCGTCATATCATAGTAGCCTGTCGAACAGGTGGATAAATCAGTCTTTCCAT CACAAGACAAGCCACAGGGTCTCCAGCTTGACCCTCGTAAAACCTGTCATCGTGATTAAACAACAGCACCGAAAGTT CCTCGCGGTGGCCAGCATGAATAATTCTTGATGAAGCATATAATCCAGACATGTTAGCATCAGTTAAAGAGAAAAAA CAGCCAACATAGCCTCTGGGTATAATTATGCTTAATCTTAAGTATAGCAAAGCCACCCCTCGCGGATACAAAGTAAA AGGCACAGGAGAATAAAAAATATAATTATTTCTCTGCTGCTGTTCAGGCAACGTCGCCCCCGGTCCCTCTAAATACA CATACAAAGCCTCATCAGCCATGGCTTACCAGACAAAGTACAGCGGGCGCACAAAGCACAAGCTCTAAAGAAGCTCT AAAGACACTCTCCAACCTCTCCACAATATATACACAAGCCCTAAACTGACGTAATGGGAGTAAAGTGTAAAAAATCC CGCCAAGCCCAACACACACCCCGAAACTGCGTCAGCAGGGAAAAGTACAGTTTCACTTCCGCAAACCCAACAAGCGT AACTTCCTCTTTCTCACGGTACGTCACATCCGATTAACTTGCAACGTCATTTTCCCACGGCCGCACCGCCCCTTTTA GCCGTTCACCCCGCAGCCAATCACCACACAGCGCGCACTTTTTTAAATTACCTCATTTACATGTTGGCACCATTCCA TCTATAAGGTATATTATTGATAATG

GenBank Accession No. AAW33461 MAKRARLSSSFNPVYPYEDESSSQHPFINPGFISSNGFAQSPDGVLTLKCVNPLTTASGPLQLKVGSSLTVDTIDGS LEENITAAAPLTKTNHSIGLLIGSGLQTKDDKLCLSLGDGLVTKDDKLCLSLGDGLITKNDVLCAKLGHGLVFDSSN AITIENNTLWTGAKPSANCVIKEGEDSPDCKLTLVLVKNGGLINGYITLMGASEYTNTLFKNNQVTIDVNLAFDNTG QIITYLSSLKSNLNFKDNONMATGTITSAKGFMPSTTAYPFITYATETLNEDYIYGECYYKSTNGTLFPLKVTVTLN RRMLASGMAYAMNFSWSLNAEEAPETTEVTLITSPFFFSYIREDD

GenBank Accession No. AAW33439 MRRRAVLGGAVVYPEGPPPSYESVMQQQAAMIOPPLEAPFVPPRYLAPTEGRNSIRYSELSPQYDTTKLYLVDNKSA DIASLNYQNDHSNFLTTVVONNDFTPTEASTQTINFDERSRWGGQLKTIMHTNMPNVNEYMFSNKFKARVMVSRKAP EGVTVNDHKDDILKYEWFEFTLPEGNFSATMTIDLMNNAIIDNYLKIGRONGVLESDIGVKFDTRNFRLGWDPETKL IMPGVYTYEAFHPDIVLLPGCGVDFTESRLSNLLGIRKRHPFQEGFKIMYEDLEGGNIPALLDVTAYEESKKDTTTE TGEKAVVKTTTVAVAEETSEDDNITRGDTYITEKQKREAAAAELLLMSEVKKELKIQPLEKDSKNRSYNVLEDKINT AYRSWYLSYNYGNPEKGIRSWTLLTTSDVTCGAEQVYWSLPDMMQDPITFRSSRQVNNYPVVGAELMPVFSKSFYNE QAVYSQQLRQSTSLTHVENREPENQILIRPPAPTITTISENVPALTDHGTLPLRSSIRGVQRVTVTDARRRTCPYVY KALGIVAPRVLSSRTF

GenBank Accession No. AAW33444 MATPSMMPQWAYMHIAGQDASEYLSPGLVQFARATDTYFSMGNKFRNPTVAPTHDVTTDRSQRLMLRFVPVDREDNT YSYKVRYTLAVGDNRVLDMASTFFDIRGVLDRGPSFKPYSGTAYNSLAPKGAPNTCQWKDSDSKMHTFGVAAMPGVT GKKIEADGLPIGIDSTSGTDTVIYADKTFQPEPQVGNASWVDANGTEEKYGGRALKDTTKMKPCYGSFAKPTNKEGG QANLKDSETAATTPNYDIDLAFFDNKNIAANYDPDIVMYTENVDLQTPDTHIVYKPGTEDTSSESNLGQQAMPNRPN YIGFRDNFIGLMYYNSTGNMGVLAGQASQLNAVVDLQDRNTELSYQLLLDSLGDRTRYFSMWNQAVDSYDPDVRIIE NHGVEDELPNYCFPLNGVGFTDTYQGVKVKTDAVAGTSGTQWDKDDTTVSTANEIHGGNPFAMEINIQANLWRSFLY SNVALYLPDSYKYTPSNVTLPENKNTYDYMNGRVVPPSLVDTYVNIGARWSLDAMDNVNPFNHHRNAGLRYRSMLLG NGRYVPFHIQVPQKFFAVKNLLLLPGSYTYEWNFRKDVNMVLQSSLGNDLRVDGASISFTSINLYATFFPMAHNTAS TLEAMLRNDTNDQSENDYLSAANMLYPIPANATNIPISIPSRNWAAFRGWSFTRLKTKETPSLGSGFDPYFVYSGSI PYLDGTFYLNHTFKKVSIMFDSSVSWPGNDRLLSPNEFEIKRTVDGEGYNVAQCNMTKDWFLVOMLANYNIGYQGFY IPEGYKDRMYSFERNFQPMSRQVVDEVNYTDYKAVTLPYQHNNSGFVGYLAPTMROGEPYPANYPYPLIGTTAVKSV TOKKFLCDRTMWRIPFSSNFMSMGALTDLGONLLYANSAHALDMTFEVDPMDEPTLLYLLFEVFDVVRVHQPHRGVI EAVYLRTPFSAGNATT

GenBank Accession No. AY601633 (SEQ ID NO: 214) CTATCTATATAATATACCTTATAGATGGAATGGTGCCAATATGCAAATGAGGTAATTTAAAAAAGTGCGCGCTGTGT GGTGATTGGCTGCGGGGTGAACGGCTAAAAGGGGGGGGCAATGCTGGGAGGTGACGTAACTTATGTAGGAGGAGTTA TGTTGCAAGTTATCGCGGTAAAGGTGACGTAAAACGAGGTGTGGTTTGGACACGGAAGTAGACAGTTTTCCCACGCT TACTGACAGGATATGAGGTAGTTTTGGGCGGATGCAAGTGAAAATTCTCCATTTTCGCGCGAAAACTGAATGAGGAA GTGAATTTCTGAGTCATTTCGCGGTTATGACAGGGTGGAGTATTTGCCGAGGGCCGAGTAGACTTTGACCGTTTACG TGGAGGTTTCGATTACCGTGTTTTTCACCTAAATTTCCGCGTACGGTGTCAAAGTCCTGTGTTTTTACGTAGGTGTC AGCTGATCACTAGGGTATTTAAACCTGTCGAGTTCCGTCAAGAGGCCACTCTTGAGTGCCAGCGAGAAGAGTTTTCT CCTCCGCGCTGCGAGTCAGTTTTGCGCTTTGAAAATGAGACACCTGCGATTCCTGCCACAGGAGATTATCTCCAGCG AGACCGGGATCGAAATACTGGAGTTTGTGGTAAATACCCTGATGGGAGATGACCCGGAACCGCCAGTGCAGCCTTTC GATCCACCTACGCTGCACGATCTGTATGATTTAGAGGTAGACGGGCCTGATGATCCCAATGAGGAAGCTGTAAATGG GTTTTTTACTGATTCTATGCTGCTAGCTGCCGATGAAGGATTGGACATAAACCCTCCTCCTGGGACCCTTGATACCC CAGGGGTGGTTGTGGAAAGCGGCAGAGGTGGGAAAAAATTGCCTGATCTGGGAGCAGCTGAAATGGACTTGCGTTGT TATGAAGAGGGTTTTCCTCCGAGTGATGATGAAGATGGGGAAACTGAACAGTCCATCCATACCGCAGTGAATGAGGG AGTAAAAGCTGCCAGCGATGTTTTTAAGTTGGACTGTCCGGAGCTGCCTGGACATGGCTGTAAGTCTTGTGAATTTC ACAGGAATAACACTGGAATGAAAGAACTATTGTGCTCGCTTTGCTATATGAGAATGCACTGCCACTTTATTTACAGT AAGTGTATTTAAGTGAAATTTAAAGGAATAGTGTAGCTGTTTAATAACTGTTGAATGGTAGATTTATGTTTTTACTT GCGATTTTTTGTAGGTCCTGTGTCTGATGATGAGGCGCCTTCTCCTGATTCAACTACCTCACCTCCTGAAATTCAGG CGCCCGTACCTGCAAACGTATGCAAGCCCATTCCTGTGAAGCCTAAGTGTGGGAAACGCCCTGCTGTGGATAAGCTT GAGGACTTGTTGGAGGGTGGGGATGGACCTTTGGACCTTAGTACCCGGAAACTGCCAAGACAATGAGTGCCCTGCAG CTGTGTTTATTTAATGTGACGTCATGTAATAAAATTATGTCAGCTGCTGAGTGTTTTATTGCTTCTTGGGTGGGGAC TTGGATATATAAGTAGGAGCAGATCTGTGTGGTTAGCTCATAGCAACCTGCTGCCATCCATGGAGGTTTGGGCTATC TTGGAAGACCTGAGACAGACTAGGCTACTGCTAGAAAACGCCTCGGACGGAGTCTCTGGCTTTTGGAGATTCTGGTT CGGTGGTGATCTAGCTAGGCTAGTGTTTAGGATAAAACAGGACTACAGGGAAGAATTTGAAAAGTTATTGGACGACA GTCCAGGACTTTTTGAAGCTCTTAACTTGGGCCACCAGGCTCATTTTAAGGAGAAGGTTTTATCAGTTTTAGATTTT TCTACTCCTGGTAGAACTGCTGCTGCTGTAGCTTTTCTTACTTTTATATTGGATAAATGGATCCGCCAAACCCACTT CAGCAAGGGATACGTTTTGGATTTCATAGCAGCAGCTTTGTGGAGAACATGGAAGGCTCGCAGGATGAGGACAATCT TAGATTACTGGCCAGTGCAGCCTCTGGGAGTAGCAGGGATACTGAGACACCCACCGGCCATGCCAGCGGTTCTGGAG GAGGAGCAGCAGGAGGACAATCCGAGAGCCGGCCTGGACCCTCCGGTGGAGGAGTAGCTGACCTGTTTCCTGAACTG CGACGGGTGCTTACTAGGTCTACGTCCAGTGGACAGGACAGGGGCATTAAGAGGGAGAGGAATCCTAGTGGGAATAA TTCAAGAACCGAGTTGGCTTTAAGTTTAATGAGCCGTAGGCGTCCTGAAACTGTTTGGTGGCATGAGGTTCAGAGCG AAGGCAGGGATGAAGTTTCAATATTGCAGGAGAAATATTCACTAGAACAACTTAAGACCTGTTGGTTGGAACCTGAG GATGATTGGGAGGTGGCCATTAGGAATTATGCTAAGATATCTCTGAGGCCTGATAAACAGTATAGAATTACTAAGAA GATTAATATTAGAAATGCATGCTACATATCAGGGAATGGGGCAGAGGTTATAATAGATACCCAAGATAAAGCAGCTT TTAGATGTTGTATGATGGGTATGTGGCCAGGGGTTGTCGGCATGGAAGCAGTAACATTTATGAATATTAGGTTTAAA GGGGATGGGTATAATGGCATTGTATTTATGGCTAACACTAAGCTGATTCTACATGGTTGTAGCTTTTTTGGGTTTAA TAATACTTGTGTAGAAGCTTGGGGGCAAGTTGGTGTGAGGGGTTGTAGTTTTTATGCATGCTGGATTGCAACATCAG GTAGGGTCAAGAGTCAGTTGTCTGTGAAGAAATGCATGTTTGAGAGATGTAATCTTGGCATACTGAATGAAGGTGAA GCAAGGGTCCGCCACTGCGCAGCTACAGAAACTGGCTGCTTCATTCTAATAAAGGGAAATGCCAGTGTGAAGCATAA TATGATCTGTGGACATTCGAATGAGAGGCCTTATCAGATGCTGACCTGCGCTGGTGGACATTGCAATATTCTTGCTA CCGTGCATATCGTTTCCCATGCACGCAAGAAATGGCCTGTATTTGAACATAATGTGATTACCAAGTGCACCATGCAC ATAGGTGGTCGCAGGGGAATGTTTATGCCTTACCAGTGTAACATGAATCATGTGAAGGTGATGTTGGAACCAGATGC CTTTTCCAGAGTGAGCTTAACAGGAATCTTTGATATGAATATTCAACTATGGAAGATCCTGAGATATGATGACACTA AACCGAGGGTGCGCGCATGCGAATGCGGAGGCAAGCATGCTAGATTCCAGCCGGTGTGCGTGGATGTGACTGAAGAC CTGAGACCCGATCATTTGGTGCTTGCCTGCACTGGAGCGGAGTTCGGTTCTAGTGGTGAAGAAACTGACTAAAGTGA GTAGTGGGGCAATATGTGGATGGGGACTTTCAGGTTGGTAAGGTGGACAGATTGGGTAAATTTTGTTAATTTCTGTC TTGCAGCTGCCATGAGTGGAAGCGCTTCTTTTGAGGGGGGAGTATTTAGCCCTTATCTGACGGGCAGGCTCCCATCA TGGGCAGGAGTTCGTCAGAATGTCATGGGATCCACTGTGGATGGGAGACCCGTCCAGCCCGCCAATTCCTCAACGCT GACCTATGCCACTTTGAGTTCGTCATCATTGGATGCAGCTGCAGCCGCCGCCGCTACTGCTGCCGCCAACACCATCC TTGGAATGGGCTATTACGGAAGCATCGTTGCCAATTCCAGTTCCTCTAATAACCCTTCAACCCTGGCTGAGGACAAG CTGCTTGTTCTCTTGGCTCAGCTCGAGGCCTTAACCCAACGCTTAGGCGAACTGTCTAAGCAGGTGGCCCAGTTGCG TGAGCAAACTGAGTCTGCTGTTGCCACAGCAAAGTCTAAATAAAGATCTCAAATCAATAAATAAAGAAATACTTGTT ATAAAAACAAATGAATGTTTATTTGATTTTTCGCGCGCGGTATGCCCTGGACCATCGGTCTCGATCATTGAGAACTC GGTGGATCTTTTCCAGTACCCTGTAAAGGTGGGATTGAATGTTTAGATACATGGGCATTAGTCCGTCTCGGGGGTGG AGATAGCTCCATTGAAGAGCCTCTTGCTCCGGGGTAGTGTTATAAATCACCCAGTCATAGCAAGGTCGGAGTGCATG GTGTTGCACAATATCTTTTAGGAGCAGACTAATTGCAACGGGGAGGCCCTTAGTGTAGGTGTTTACAAATCTGTTGA GCTGGGACGGGTGCATCCTGGGTGAAATTATATGCATTTTGGACTGGATCTTGAGGTTGGCAATGTTGCCGCCTAGA TCCCGTCTCGGATTCATATTGTGCAGAACCACCAAGACAGTGTATCCGGTGCACTTGGGAAATTTATCATGCAGCTT AGAGGGAAAAGCATGAAAAAATTTGGAGACGCCTTTGTGACCCCCCAGATTCTCCATGCACTCATCCATAATGATAG CGATGGGGCCGTGGGCAGCGGCACGGGCGAACACGTTCCGGGGGTCTGAAACATCATAGTTATGCTCCTGAGTCAGG TCATCATAAGCCATTTTAATAAACTTTGGGCGGAGGGTGCCAGATTGGGGGATGAAAGTTCCCTCTGGCCCGGGAGC ATAGTTTCCCTCACATATTTGCATTTCCCAGGCTTTCAGTTCCGAGGGGGGGATCATGTCCACCTGCGGGGCTATAA AAAATACCGTTTCTGGAGCCGGGGTGATTAACTGGGATGAGAGCAAATTCCTAAGCAGCTGAGACTTGCCGCACCCA GTGGGACCGTAAATGACCCCAATTACGGGTTGCAGATGGTAGTTTAGGGAGCGACAGCTGCCGTCCTCCCGGAGCAG GGGGGCCACTTCGTTCATCATTTCCCTTACATGGATATTTTCCCGCACCAAGTCCGTTAGGAGGCGCTCTCCCCCAA GGGATAGAAGCTCCTGGAGCGAGGAGAAGTTTTTCAGCGGCTTCAGCCCGTCAGCCATGGGCATTTTGGAAAGAGTC TGTTGCAAGAGCTCGAGCCGGTCCCAGAGCTCGGTGATGTGCTCTATGGCATCTCGATCCAACAGACCTCCTCGTTT CGCGGGTTGGGACGGCTCCTGGAGTAGGGAATCAGACGATGGGCGTCCAGCGCTGCTAGGGTCCGATCCTTCCATGG TCGCAGCGTCCGAGTCAGGGTTGTTTCCGTCACGGTGAAGGGGTGCGCGCCTGGTTGGGCGCTTGCGAGGGTGCGCT TCAGACTCATCCTGCTGGTCGAGAACCGCTGCCGATCGGCGCCCTGCAGGTCGGCCAGGTAGCAGTTTACCATGAGT TCGTAGTTGAGCGCCTCGGCCGCGTGGCCTTTGGCACGGAGCTTACCTTTGGAAGTTTTATGGCAGGCGGGGCAGTA GATACATTTGAGGGCATACAGCTTGGGCGCGAGGAAAATGGATTCGGGGGAGTATGCATCCGCACCGCAGGAGGCGC AGACGGTTTCGCACTCCACGAGCCAGGTCAGATCCGGCTCATCGGGGTCAAAAACAAGTTTTCCGCCATGTTTTTTG ATGCGTTTCTTACCTTTGGTTTCCATGAGTTCGTGTCCCCGCTGGGTGACAAAGAGGCTGTCCGTGTCCCCGTAGAC CGACTTTATGGGCCTGTCCTCGAGCGGAGTGCCTCGGTCCTCTTCGTAGAGGAACCCAGCCCACTCTGATACAAAAG CGCGTGTCCAGGCCAGCACAAAGGAGGCCACGTGGGAGGGGTAGCGGTCGTTGTCAACCAGTGGGTCCACCTTCTCT ACGGTATGTAAACACATGTCCCCCTCCTCCACATCCAAGAATGTGATTGGCTTGTAAGTGTAGGCCACGTGACCAGG GGTCCCCGCCGGGGGGGTATAAAAGGGGGCGGGCCTCTGTTCGTCCTCACTGTCTTCAGGATCGCTGTCCAGGAGCG CCAGCTGTTGGGGTAGGTATTCCCTCTCGAAGGCGGGCATGACCTCTGCACTCAGGTTGTCAGTTTCTAGGAACGAG GAGGATTTGATATTGACAGTACCAGCAGAGACGCCTTTCATAAGACTCTCGTCCATCTGGTCAGAAAACACAATCTT CTTGTTGTCCAGCTTGGTGGCAAATGATCCATAAAGGGCATTGGACAGAAGCTTGGCGATGGAGCGCATGGTTTGGT TCTTTTCCTTGTCCGCGCGCTCCTTGGCGGCGATGTTAAGCTGGACGTACTCGCGCGCCACACATTTCCATTCAGGG AAGATGGTTGTCAGTTCATCCGGAACTATTCTGACTCGCCATCCCCTATTGTGCAGGGTTATCAGATCCACACTGGT GGCCACCTCGCCTCGGAGGGGCTCATTGGTCCAGCAGAGTCGACCTCCTTTTCTTGAACAGAAAGGTGGGAGGGGGT CTAGCATGAACTCATCAGGGGGGTCCGCATCTATGGTAAATATTCCCGGTAGCAAATCTTTGTCAAAATAGCTGATG GTGGCGGGATCATCCAAGGTCATCTGCCATTCTCGAACTGCCAGCGCGCGCTCATAGGGGTTAAGAGGGGTGCCCCA GGGCATGGGGTGGGTGAGCGCGGAGGCATACATGCCACAGATATCGTAGACATAGAGGGGCTCTTCGAGGATGCCGA TGTAAGTGGGATAACAGCGCCCCCCTCTGATGCTTGCTCGCACATAGTCATAGAGTTCATGTGAGGGGGCGAGAAGA CCCGGGCCCAGATTGGTGCGGTTGGGTTTTTCCGCCCTGTAAACGATCTGGCGAAAGATGGCATGGGAATTGGAAGA GATAGTAGGTCTCTGGAATATGTTAAAATGGGCATGAGGTAGGCCTACAGAGTCCCTTATGAAGTGGGCATATGACT CTTGCAGCTTGGCTACCAGCTCGGCGGTGACGAGTACATCCAGGGCACAGTAGTCGAGAGTTTCCTGGATGATGTCA TAACGCGGTTGGCTTTTCTTTTCCCACAGCTCGCGGTTGAGAAGGTATTCTTCGCGATCCTTCCAGTACTCTTCGAG GGGAAACCCGTCTTTTTCTGCACGGTAAGAGCCCAACATGTAGAACTGATTGACTGCCTTGTAGGGACAGCATCCCT TCTCCACTGGAAGAGAGTATGCTTGGGCTGCATTGCGCAGCGAGGTATGAGTGAGGGCAAAAGTGTCCCTGACCATG ACTTTGAGGAATTGATACTTGAAGTCAATGTCATCACAGGCCCCCTGTTCCCAGAGTTGGAAGTCCACCCGCTTCTT GTAGGCGGGGTTGGGCAAAGCGAAAGTAACATCATTGAAGAGGATCTTGCCGGCCCTGGGCATGAAATTTCGGGTGA TTTTGAAAGGCTGAGGGACCTCTGCTCGGTTATTGATAACCTGAGCGGCCAAGACGATCTCATCAAAGCCATTGATG TTGTGCCCCACTATGTACAGTTCTAAGAATCGAGGGGTGCCCCTGACATGAGGCAGCTTCTTGAGTTCTTCAAAAGT GAGATCTGTAGGGTCAGTGAGAGCATAGTGTTCGAGGGCCCATTCGTGCACGTGAGGGTTCGCTTTGAGGAAGGAGG ACCAGAGGTCCACTGCCAGTGCTGTTTGTAACTGGTCCCGGTACTGACGAAAATGCTGCCCGACTGCCATCTTTTCT GGGGTGACGCAATAGAAGGTTTGGGGGTCCTGCCGCCAGCGATCCCACTTGAGTTTCATGGCGAGGTCATAGGCGAT GTTGACGAGCCGCTGGTCTCCAGAGAGTTTCATGACCAGCATGAAGGGGATTAGCTGCTTGCCAAAGGACCCCATCC AGGTGTAGGTTTCCACATCGTAGGTGAGGAAGAGCCTTTCTGTGCGAGGATGAGAGCCAATCGGGAAGAACTGGATC TCCTGCCACCAGTTGGAGGAATGGCTGTTGATGTGATGGAAGTAGAACTCCCTGCGACGCGCCGAGCATTCATGCTT GTGCTTGTACAGACGGCCGCAGTACTCGCAGCGATTCACGGGATGCACCTCATGAATGAGTTGTACCTGACTTCCTT TGACGAGAAATTTCAGTGGAAAATTGAGGCCTGGCGTTTGTACCTCGCGCTCTACTATGTTGTCTGCATCGGCATGA CCATCTTCTGTCTCGATGGTGGTCATGCTGACGAGCCCTCGCGGGAGGCAAGTCCAGACCTCGGCGCGGCAGGGGCG GAGCTCGAGGACGAGAGCGCGCAGGCCGGAGCTGTCCAGGGTCCTGAGACGCTGCGGAGTCAGGTTAGTAGGCAGTG TCAGGAGATTGACTTGCATGATCTTTTCGAGGGCGTGAGGGAGGTTCAGATGGTACTTGATCTCCACGGGTCCGTTG GTGGAGATGTCGATGGCTTGCAGGGTTCCGTGCCCCTTGGGTGCTACCACCGTGCCCTTGTTTTTCCTTTTGGGCGG CGGTGGCTCTGTTGCTTCTTGCATGTTTAGAAGCGGTGTCGAGGGCGCGCACCGGGCGGCAGGGGCGGTTCGGGACC CGGCGGCATGGCTGGCAGTGGTACGTCGGCGCCGCGCGCGGGTAGGTTCTGGTACTGTGCTCTGAGAAGACTCGCAT GCGCGACGACGCGGCGGTTGACATCCTGGATCTGACGCCTCTGGGTGAAAGCTACCGGCCCCGTGAGCTTGAACCTG AAAGAGAGTTCAACAGAATCAATCTCGGTATCGTTGACGGCGGCTTGCCTAAGGATTTCTTGCACGTCGCCAGAGTT GTCCTGGTAGGCGATCTCGGCCATGAACTGCTCGATCTCTTCCTCTTGAAGATCTCCGCGGCCCGCTCTCTCGACGG TGGCCGCCAGGTCGTTGGAGATGCGCCCAATGAGTTGAGAGAATGCATTCATGCCCGCCTCGTTCCAGACGCGGCTG TAGACCACAGCCCCCACGGGATCTCTCGCGCGCATGACCACCTGGGCGAGGTTGAGCTCCACGTGGCGGGTGAAGAC CGCATAGTTGCATAGACGCTGGAAAAGGTAGTTGAGTGTGGTGGCGATGTGCTCGGTGACGAAGAAATACATGATCC ATCGTCTCAGCGGCATCTCGCTGACATCGCCCAGCGCTTCCAAGCGCTCCATGGCCTCGTAGAAGTCCACGGCAAAG TTGAAAAACTGGGAGTTACGCGCGGACACGGTCAACTCCTCTTCCAGAAGACGGATGAGTTCGGCGACGGTGGTGCG CACCTCGCGCTCGAAAGCCCCTGGGATTTCTTCCTCAATCTCTTCTTCTTCCACTAACATCTCTTCCTCTTCAGGTG GGGCTGCAGGAGGAGGGGGAACGCGGCGACGCCGGCGGCGCACGGGCAGACGGTCGATGAATCTTTCAATGACCTCT CCGCGGCGGCGGCGCATGGTCTCGGTGACGGCACGACCGTTCTCCCTGGGTCTCAGAGTGAAGACGCCTCCGCGCAT CTCCCTGAAGTGGTGACTGGGAGGCTCTCCGTTGGGCAGGGACACCGCGCTGATTATGCATTTTATCAATTGCCCCG TAGGTACTCCGCGCAAGGACCTGATCGTCTCAAGATCCACGGGATCTGAAAACCTTTCGACGAAAGCGTCTAACCAG TCGCAATCGCAAGGTAGGCTGAGCACTGTTTCTTGCGGGGGGGGCGGCTAGACGCTCGGTCGGGGTTCTCTCTTTC TTCTCCTTCCTCCTCTTTGGAGGGTGAGACGATGCTGCTGGTGATGAAATTAAAATAGGCAGTTTTGAGACGGCGGA TGGTGGCGAGGAGCACCAGGTCTTTGGGTCCGGCTTGTTGGATGCGCAGGCGATGGGCCATTCCCCAAGCATTATCC TGACATCTGGCCAGATCTTTATAGTAGTCTTGCATGAGTCGTTCCACGGGCACTTCTTCTTCGCCCGCTCTGCCATG CATGCGAGTGATCCCGAACCCGCGCATGGGCTGGACAAGTGCCAGGTCCGCTACAACCCTTTCTGCGAGGATGGCTT GCTGCACCTGGGTGAGGGTGGCTTGGAAGTCGTCAAAGTCCACGAAGCGGTGGTAAGCCCCGGTGTTGATTGTGTAG GAGCAGTTGGCCATGACTGACCAGTTGACTGTCTGGTGCCCCGGACGCACAATCTCGGTGTACTTGAGGCGCGAGTA GGCGCGGGTGTCAAAGATGTAATCGTTACAGGTGCGCACCAGGTACTGGTAGCCGATAAGAAAGTGCGGCGGCGGCT GGCGGTATAGGGGCCATCGCTCTGTAGCCGGGGCGCCAGGGGCGAGGTCTTCCAGCATGAGGCGGTGATAACCGTAG ATGTACCTGGACATCCAGGTGATACCGGAGGCGGTGGTGGATGCCCGCGGGAACTCGCGTACGCGGTTCCAGATGTT GCGCAGCGGCATGAAGTAGTTCATGGTAGGCACGGTTTGGCCCGTGAGACGTGCACAGTCGTTGATGCTCTAGACAT ACGGGCAAAAACGAAAGCGGTCAGCGGCTCGTCTCCGTGGCCTGGAGGCTAAGCGAACGGGTTGGGCTGCGCGTGTA CCCCGGTTCGAATCTCGGATCAGGCTGGAGCCGCAGCTAACGTGGTACTGGCACTCCCGTCTCGACCCAGGCCTGCA CAAAACCTCCAGGATACGGAGGCGGGTCGTTTTTTTTTTTTTTTGCTTTTTCCTGGATGGGAGCCAGTGCTGCGTCA AGCTTTAGAACACTCAGTTCTCGGGGCTGGGAGTGGCTCGCGCCCGTAGTCTGGAGAATCAATCGCCAGGGTTGCGT TGCGGTGTGCCCCGGTTCGAGTCTTAGCGCGCCGGATCGGCCGGTTTCCGCGACAAGCGAGGGTTTGGCAGCCCCGT CATTTCTAAGACCCCGCCAGCCGACTTCTCCAGTTTACGGGAGCGAGCCCTCTTTTTTTGTTTTTTTGTTGCCCAGA TGCATCCCGTGCTGCGACAGATGCGCCCCCAGCAACAGCCCCCTTCTCAGCAGCAGCTACAACAACAGCCACAAAAG GCTCTTCCTGCTCCTGTAACTACTGCGGCTGCAGCCGTCAGCGGCGCGGGGCAGCCCGCCTATGATCTGGACTTGGA AGAGGGCGAGGGACTGGCGCGCCTGGGCGCACCATCGCCCGAGCGGCACCCGCGGGTGCAACTGAAAAAGGACTCTC GCGAGGCGTACGTGCCCCAGCAGAACCTGTTCAGGGACAGGAGCGGCGAGGAGCCTGAGGAAATGCGAGCTTCCCGC TTTAACGCGGGTCGCGAACTGCGTCACGGTCTGGACCGAAGACGGGTGCTGCGTGATGATGATTTTGAAGTCGATGA AGTGACAGGAATAAGTCCTGCTAGGGCACATGTGGCCGCGGCCAACCTAGTATCAGCTTACGAGCAGACCGTGAAGG AGGAGCGCAACTTTCAAAAATCTTTCAACAACCATGTGCGCACCCTGATTGCCCGCGAGGAAGTGACACTGGGACTG ATGCACCTGTGGGACCTGATGGAAGCCATTACCCAGAACCCCACCAGCAAACCTCTAACCGCTCAGCTGTTTCTGGT GGTGCAACATAGTAGAGACAATGAGGCATTTAGGGAGGCGCTGTTGAACATTACTGAGCCCGAGGGGAGATGGTTGT ATGATCTTATCAATATTCTGCAAAGTATAATAGTGCAAGAACGTAGCCTGGGTCTAGCTGAGAAGGTGGCTGCTATT AACTACTCGGTCTTGAGCCTGGGCAAGCACTACGCTCGCAAGATCTACAAAACCCCATACGTACCTATAGACAAGGA GGTGAAGATAGATGGGTTTTATATGCGCATGACTCTCAAGGTGCTGACCTTGAGTGACGATCTGGGAGTGTACCGCA ACGACAGGATGCACCGCGCAGTGAGCGCCAGCAGAAGGCGTGAGCTGAGCGACAGAGAACTTATGCACAGCTTGCAA AGAGCTCTGACTGGGGCTGGAACCGAGGGGGAGAACTACTTTGACATGGGAGCGGACTTGCAATGGCAGCCCAGTCG CAGGGCCCTGGACGCAGCAGGGTATGAGCTTCCTTACATAGAAGAGGTGGATGAAGGCCAGGATGAGGAGGGCGAGT ACCTGGAAGACTGATGGCGCGACCATCCATATTTTTGCTAGATGGAACAGCAGGCACCGGACCCCGCAAAACGGGCG GCGCTACAGAGCCAGCCGTCCGGCATTAACTCCTCGGACGATTGGAGCCAGGCCATGCAACGCATCATGGCGCTGAC GACCCGCAACCCCGAAGCCTTTAGGCAGCAACCCCAGGCCAACCGCCTTTCTGCTATCCTGGAGGCCGTAGTGCCCT CCCGCTCCAACCCCACACACGAGAAGGTCCTGGCCATCGTGAACGCGCTGGTGGAGAACAAAGCCATACGTCCCGAT GAGGCTGGGCTGGTATACAATGCCCTATTGGAGCGCGTAGCCCGCTACAACAGCAGCAACGTGCAGACCAACCTGGA CCGGATGGTGACCGATGTGCGCGAGGCCGTGTCTCAGCGCGAGCGGTTCCAGCGAGACGCCAATTTAGGGTCGCTGG TGGCTTTGAACGCCTTCCTCAGCACTCAGCCTGCCAACGTGCCTCGCGGTCAGCAAGACTACACAAACTTTCTAAGT GCATTGAGACTCATGGTGGCCGAAGTCCCTCAAAGCGAAGTGTACCAGTCCGGGCCAGACTACTTTTTCCAGACCAG CAGACAGGGCTTGCAGACAGTGAACCTGAGCCAGGCTTTTAAGAACCTGAATGGTCTGTGGGGAGTGCGCGCCCCAG TGGGAGATCGGGCGACCGTGTCTAGCTTGCTGACCCCCAACTCCCGCCTACTACTGCTCTTGGTAGCCCCATTCACT GACAGCGGTAGCATCGACCGTAATTCGTACTTGGGCTATCTGTTGAACCTGTATCGCGAGGCCATAGGGCAAACTCA GGTAGATGAGCAAACCTATCAAGAAATTACCCAAGTGAGCCGCGCTCTGGGTCGGGAGGACACTGGCAGCTTGGAAG CCACCTTAAACTTCTTGCTGACCAACCGGTCGCAGAAGATCCCTCCTCAGTATGCGCTTACCGCGGAGGAGGAACGG ATCCTGAGATACGTGCAGCAGAGCGTGGGACTGTTCCTAATGCAGGAGGGGGCGACTCCTACTGCTGCGCTCGATAT GACAGCCCGAAACATGGAGCCCAGCATGTATGCCAGTAACCGGCCTTTTATCAATAAACTGCTAGACTACTTACACA GGGCGGCTGCTATGAACTCTGATTATTTCACCAATGCTATCCTGAACCCCCATTGGCTGCCCCCACCTGGGTTCTAT ACGGGCGAGTATGACATTGCCCGACCCAATGACGGGTTTTTATGGGACGATGTGGACAGTAGTGTTTTCTCCCCGCC TCCTGGTTATAACACTTGGAAGAAGGAAGGTGGCGATAGAAGGCACTCTTCCGTGTCACTGTCCGGGGCAACGGGTG CTGCCGCAGCGGCTCCCGAGGCCGCAAGTCCTTTCCCTAGTTTGCCATTTTCGCTAAACAGTGTACGCAGCAGTGAG CTGGGAAGAATAACCCGTCCTCGCTTGATCGGCGAGGAGGAGTATTTGAACGACTCCCTGTTGAGACCCGAGAGGGA GAAGAATTTCCCCAACAACGGGATAGAAAGCTTGGTTGACAAAATGAACCGCTGGAAGACGTACGCGCACGATCCCC GGGCGCTGGGGGATAGCCGGGGCAGCGCTACCCGTAAACGCCAGTGGCACGACAGGCAGCGGGGCCTGGTGTGGGCC GATGATGATTCCGCCGACGACAGCAGCGTGTTGGACTTGGGTGGGAGTGGTGGTAACCCGTTCGCTCACCTGCGCCC CCGCGTCGGGCGCCTGATGTAAGAAACCGAAAATAAATACTCACCAAGGCCATGGCGACCAGCGTGCGTTCGTTTCT TCTCTGTTATATCTAGTATGATGAGGCGAACCGTGCTAGGCGGAGCGGTGGTGTATCCGGAGGGTCCTCCTCCTTCG TACGAGAGCGTGATGCAGCAGGCGGCGGCGGCGACGATGCAGCCACCACTGGAGGCTCCCTTTGTACCCCCTCGGTA CCTGGCACCTACGGAGGGGAGAAACAGCATTCGTTACTCGGAGCTGGCACCATTGTATGATACCACCCGGTTGTATT TGGTGGACAACAAGTCCGCGGACATCGCCTCACTGAACTATCAGAACGACCACAGCAACTTCCTCACCACGGTGGTG CAAAACAATGACTTTACCCCCACGGAGGCCAGCACCCAGACCATCAACTTTGACGAGCGGTCGCGATGGGGCGGTCA GCTGAAGACTATCATGCACACCAACATGCCCAACGTGAACGAGTACATGTTTAGCAACAAGTTCAAAGCTCGGGTGA TGGTGTCTAGAAAGGCTCCTGAAGGTGTCACAGTAGATGACAATTATGATCACAAGCAGGATATTTTGGAATATGAG TGGTTTGAGTTTACTCTACCGGAAGGGAATTTCTCAGCCACAATGACCATTGACCTAATGAACAATGCCATCATTGA TAATTACCTTGAAGTGGGCAGACAGAATGGAGTGTTAGAGAGTGACATTGGTGTTAAATTTGACACCAGGAACTTTA GACTGGGTTGGGATCCGGAAACTAAGTTGATTATGCCTGGGGTTTACACCTATGAGGCATTCCATCCTGACATTGTA TTGTTGCCTGGTTGCGGAGTTGACTTTACTGAAAGTCGCCTTAGTAACTTGCTTGGTATCAGGAAAAGACACCCATT CCAGGAGGGTTTTAAGATCTTGTATGAGGATCTTGAAGGGGGTAATATCCCGGCCCTGTTGGATGTAGAAGCCTATG AGAACAGTAAGAAAGAACAAGAAGCCAAAACAGAAGCCGCTAAAGCTGCTGCTATTGCTAAAGCCAACATAGTTGTC AGCGACCCTGTAAGGGTGGCTAATGCAGAAGAAGTCAGAGGAGACAACTATACAGCTTCATCTGTTGCAACTGACGA ATCGCTATTGGCTGCTGTGGCCGAAACTACAGAGACAAAACTCACTATTAAACCTGTAGAAAAAGACAGCAAGAGTA GAAGTTACAATGTCTTGGAAGATAAAGTGAATACAGCCTACCGCAGCTGGTACCTGTCCTACAACTATGGTGACCCT GAAAAAGGAGTCCGTTCCTGGACACTGCTCACCACCTCGGATGTCACCTGTGGAGCAGAGCAGGTGTACTGGTCGCT CCCAGACATGATGCAGGACCCTGTCACATTCCGTTCCACGAGACAAGTCAGCAACTATCCAGTGGTAGGTGCAGAGC TCATGCCGGTCTTCTCAAAGAGTTTCTACAACGAGCAAGCCGTGTACTCCCAGCAGCTTCGCCAGTCCACCTCGCTC ACGCACGTCTTCAACCGCTTCCCTGAGAACCAGATCCTCATCCGCCCGCCAGCGCCCACCATTACCACCGTCAGTGA AAACGTTCCTGCTCTCACAGATCACGGGACCCTGCCGTTGCGCAGCAGTATCCGGGGAGTCCAGCGCGTGACCGTTA CTGACGCCAGACGCCGCACCTGCCCCTACGTCTACAAGGCCCTGGGCATAGTCGCGCCGCGCGTCCTTTCAAGCCGC ACTTTCTAAAAAAAAAAATGTCCATTCTTATCTCACCTAGTAATAACACCGGTTGGGGCCTGCGCGCGCCAAGCAAG ATGTACGGAGGTGCTCGCAAACGCTCTACACAGCACCCTGTGCGCGTGCGCGGGCACTTCCGCGCTCCATGGGGCGC CCTCAAGGGTCGTGCCCGCACTAGAACCACCGTCGATGATGTGATCGACCAGGTGGTGGCCGATGCTCGTAATTATA CTCCTACTGCACCTACATCTACTGTGGATGCAGTTATTGACAGCGTAGTGGCTGACGCCCGCGCCTATGCTCGCCGG AAGAGCAGGCGGAGACGCATCGCCAGGCGCCACCGGGCTACTCCCGCTATGCGAGCGGCAAGAGCTCTGCTACGGAG GGCCAAACGCGTGGGGCGAAGAGCTATGCTTAGAGCGGCCAGACGCGCGGCTTCAGGTGCCAGTGCCGGCAGGTCCC GCAGGCGCGCAGCCACGGCGGCAGCAGCGGCCATTGCCAACATGGCCCAACCGCGAAGAGGCAATGTGTACTGGGTG CGCGACGCCACCACCGGCCAGCGCGTGCCCGTGCGCACCCGCCCCCCTCGCTCTTAGAAGATACTGAGCAGTCTCCG ATGTTGTGTCCCAGCGAGGATGTCCAAGCGCAAATACAAGGAAGAGATGCTCCAGGTCATCGCGCCTGAAATCTACG GTCCGCCGGTGAAGGATGAAAAAAAGCCCCGCAAAATCAAGCGGGTCAAAAAGGACAAAAAGGAAGAAGATGGCAAT GATGGTCTGGCGGAGTTTGTACGCGAGTTCGCCCCAAGGCGGCGTGTGCAGTGGCGTGGACGCAAAGTGCGGCCTGT GCTGAGACCTGGAACCACGGTGGTCTTTACGCCCGGCGAGCGCTCCAGCACTGCTTTTAAGCGGTCCTATGATGAGG TGTATGGGGATGATGATATTCTGGAGCAGGCGGCCGACCGCCTGGGCGAGTTTGCTTATGGCAAGCGCTCCCGCTCG AGCCCCAAGGAGGAGGCGGTGTCCATTCCCTTGGACAATGGGAATCCCACCCCTAGTCTCAAGCCAGTCACCCTGCA GCAAGTGCTGCCCGTGCCTCCACGCAGAGGCAACAAGCGAGAGGGTGAGGATCTGTATCCCACTATGCAATTGATGG TGCCCAAGCGCCAGCGGCTGGAGGACGTGCTGGAGAAAATGAAAGTGGATCCCGATATACAACCTGAGGTCAAAGTG AGACCCATCAAGCAGGTGGCGCCAGGTTTGGGAGTACAAACCGTAGACATCAAGATTCCCACCGAGTCCATGGAAGT CCAAACCGAACCTGCAAAGCCCACAACCACCTCCATTGAGGTGCAAACGGATCCCTGGATGACCGCACCCGTTACAA CTCCAGCTGCTGTCAACACCACTCGAAGATCCCGGCGAAAGTACGGTCCAGCAAGTTTGCTGATGCCAAATTATGCT CTGCACCCATCCATTATTCCAACTCCGGGTTACCGAGGCACTCGCTACTACCGCAGCAGGAGCAGCACTTCCCGCCG TCGCCGCAAAACACCTGCAAGTCGTAGTCACCGTCGTCGCCGCCGCCCCACCAGCAATCTGACTCCCGCTGCTCTGG TGCGGAGAGTGTATCGCGATGGCCGCGCGGATCCCCTGACGTTGCCGCGCGTACGCTACCATCCAAGCATCACAACT TAACAACTGTTGCCGCTGCCTCCTTGCAGATATGGCCCTCACTTGCCGCCTTCGTGTCCCCATTACTGGCTACCGAG GAAGAAACTCGCGCCGTAGAAGAGGGATGTTGGGGCGCGGAATGCGACGCCACAGGCGGCGGCGCGCTATCAGCAAG AGGCTGGGGGGTGGCTTTCTGCCTGCTCTGATCCCCATCATAGCCGCGGCGATCGGGGCGATACCAGGCATAGCTTC CGTGGCGGTTCAGGCCTCGCAGCGCCACTGACATTGGAAAAACTTATAAATAAAACAGAATGGACTCTGATGCTCCT GGTCCTGTGACTATGTTTTTGTAGAGATGGAAGACATCAATTTTTCATCCCTGGCTCCGCGACACGGCACGAGGCCG TACATGGGCACCTGGAGCGACATCGGCACCAGCCAACTGAACGGGGGCGCCTTCAATTGGAGCAGTATCTGGAGCGG GCTTAAAAATTTTGGCTCTACCATAAAAACCTATGGGAACAAAGCTTGGAACAGCAGCACAGGGCAGGCATTGAGAA ATAAGCTTAAAGAGCAAAACTTCCAACAGAAGGTGGTTGATGGAATCGCCTCTGGTATCAATGGGGTGGTGGATCTG GCCAACCAGGCCGTGCAGAAACAGATAAACAGCCGCATTGACCCGCCGCCGTCAGCCCCGGGTGAAATGGAAGTGGA GGAAGATCTCCCTCCCCTTGAAAAGCGGGGCGACAAGCGTCCGCGCCCCGATCTGGAGGAGACACTAGTCACACGCT CAGACGACCCGCCCTCCTACGAGGAGGCAGTGAAGCTTGGAATGCCCACCACCAGACCTGTAGCCCCCATGGCTACC GGGGTAATGAAACCTTCTCAGTCACACCGACCCGCTACCTTGGACTTGCCTCCCCCTGCTGTTGCAGCGCCTGCTCG CAAGCCTGTCGCTACCCCGAAGCCCACCACCGTACAGCCCGTCGCCGTAGCCAGACCGCGTCCTGGGGGCACTCCAC GTCCGAATGCAAACTGGCAGAGTACTCTGAACAGCATCGTGGGTCTGGGCGTGCAAAGTGTAAAGCGCCGTCGCTGC TTTTAAATTAATATGGAGTAGCGCTTAACTTGCCTGTCTGTGTGTATGTGTCATCATCACGCCGCCGCCGCAGCAAC AGCAGAGGAGCAAGGAAGAGGTCGCGCGCCGAGGCTGAGTTGATTTCAAGATGGCCACCCCATCGATGCTGCCCCAG TGGGCATACATGCACATCGCCGGACAGGATGCTTCGGAGTACCTGAGTCCGGGTCTGGTGCAGTTCGCCCGCGCCAC AGACACCTACTTCAATCTGGGGAACAAGTTTAGGAACCCCACCGTGGCGCCCACCCATGATGTGACCACCGACCGCA GTCAGCGGCTGATGCTGCGCTTTGTGCCCGTTGACCGGGAAGACAATACCTACGCATACAAAGTTCGATACACCTTG GCTGTGGGCGACAACAGAGTGCTGGATATGGCCAGCACTTTCTTTGACATTCGGGGTGTGTTGGATAGAGGCCCTAG CTTCAAGCCATACTCTGGCACTGCTTACAACTCGTTGGCCCCTAAGGGCGCTCCCAATACATCTCAGTGGATTGCTG AAGGCGTAAAAAAAGAAGATGGGGGATCTGACGAAGAGGAAGAGAAAAATCTCACCACTTACACTTTTGGAAATGCC CCAGTGAAAGCAGAAGGTGGTGATATCACTAAAGACAAAGGTCTTCCAATTGGTTCAGAAATTACAGACGGCGAAGC CAAACCAATTTATGCAGATAAACTATACCAACCAGAACCTCAGGTGGGAGATGAAACTTGGACTGACACAGATGGAA CAACTGAGAAGTATGGTGGTAGAGCTCTAAAGCCAGAAACTAAAATGAAACCCTGCTATGGGTCTTTTGCTAAACCC ACTAACGTCAAAGGCGGACAGGCAAAACAAAAAACTACTGAACAACCGCAAAACCAGCAGGTTGAATATGATATTGA CATGAACTTTTTTGATGAAGCGTCACAGAAAGCAAACTTCAGTCCAAAAATTGTGATGTATGCAGAAAATGTAGACT TGGAAACCCCAGACACTCATGTGGTGTACAAACCTGGTACTTCAGAAGAAAGTTCTCATGCTAATCTGGGTCAACAA TCTATGCCCAACAGACCCAACTACATTGGCTTTAGAGATAACTTTATTGGACTTATGTACTACAACAGTACTGGCAA CATGGGAGTGCTGGCAGGTCAAGCATCCCAATTGAATGCGGTGGTTGACTTGCAGGACAGAAACACAGAACTATCAT ATCAACTACTGCTTGACTCTCTGGGTGACAGAACCAGATACTTCAGCATGTGGAATCAAGCAGTCGATAGCTATGAT CCTGATGTGCGCATTATTGAAAATCATGGGGTGGAAGATGAGCTTCCCAACTACTGCTTTCCATTGGATGGAGTAGG GGTACCAATAAGTAGTTACAAAATAATTGAACCAAACGGACAGGGTGCAGATTGGAAAGAGCCTGACATAAATGGAA CAAGTGAAATTGGACAAGGAAATCTCTTTGCCATGGAAATTAACCTCCAAGCTAATCTCTGGAGAAGTTTTCTTTAT TCCAATGTGGCTCTGTATCTCCCAGACTCCTACAAATACACCCCAGCCAATGTCACTCTTCCAACTAACACCAACAC TTATGACTACATGAATGGGCGGGTGGTTCCCCCATCCCTGGTGGATACCTACGTAAACATTGGCGCCAGATGGTCTT TGGATGCCATGGACAATGTCAACCCCTTTAACCATCACCGCAACGCTGGCCTGCGATACCGGTCCATGCTTTTGGGC AATGGTCGTTACGTGCCTTTCCACATTCAAGTGCCTCAGAAATTCTTTGCTGTGAAGAACCTGCTGCTTCTACCCGG TTCTTACACCTACGAGTGGAACTTCAGAAAGGATGTGAACATGGTCCTGCAGAGTTCCCTTGGTAATGATCTCCGGG TCGATGGTGCCAGCATAAGTTTTACCAGCATCAATCTCTATGCCACCTTCTTCCCCATGGCCCACAACACTGCCTCC ACCCTTGAAGCCATGCTGCGCAATGACACCAATGATCAATCATTCAATGACTACCTTTCTGCTGCCAACATGCTCTA CCCCATCCCGGCCAACGCTACCAACGTTCCCATCTCCATTCCCTCTCGCAACTGGGCCGCCTTCAGAGGCTGGTCCT TCACCAGACTCAAAACCAAGGAGACTCCCTCTTTGGGATCAGGGTTCGATCCCTACTTTGTTTACTCTGGTTCTATA CCCTACCTGGATGGTACCTTCTACCTTAACCACACTTTCAAGAAAGTCTCCATCATGTTTGACTCTTCAGTGAGCTG GCCTGGTAATGACAGATTGCTAAGTCCAAATGAGTTCGAAATCAAGCGCACAGTTGATGGGGAAGGCTACAATGTGG CCCAATGTAACATGACCAAAGACTGGTTCCTGGTCCAGATGCTTGCCAACTACAACATTGGATACCAGGGCTTCTAC GTTCCTGAGGGTTACAAGGATCGCATGTACTCCTTCTTCAGAAACTTCCAGCCCATGAGTAGACAGGTGGTTGATGA GATTAACTACAAAGACTATAAAGCTGTCGCCGTACCCTACCAGCATAATAACTCTGGCTTTGTGGGTTACATGGCTC CTACCATGCGTCAGGGTCAAGCGTACCCTGCTAACTACCCATACCCCCTAATTGGAACCACTGCAGTAACCAGTGTC ACCCAGAAAAAATTCCTGTGCGACAGGACCATGTGGCGCATCCCATTCTCTAGCAACTTCATGTCCATGGGTGCCCT TACAGACCTGGGACAGAACTTGCTGTATGCCAACTCGGCCCATGCGCTGGACATGACTTTTGAGGTGGATCCCATGG ATGAGCCCACCCTGCTTTATCTTCTTTTCGAAGTCTTCGACGTGGTCAGAGTGCACCAGCCACACCGCGGCGTCATC GAGGCCGTCTACCTGCGCACACCGTTCTCGGCCGGCAACGCCACCACATAAGAAGCCTCTTGCTTCTTGCAAGCAGC AGCTGCAGCCATGTCATGCGGGTCCGGAAACGGCTCCAGCGAGCAAGAGCTCAAAGCCATCGTCCGAGACCTGGGCT GCGGACCCTATTTCCTGGGAACCTTTGACAAGCGTTTCCCGGGGTTCATGGCCCCCGACAAGCTCGCCTGCGCCATA GTCAACACTGCCGGACGCGAGACGGGGGGAGAGCACTGGCTGGCTTTTGGTTGGAACCCGCGCTCCAACACCTGCTA CCTTTTTGATCCTTTTGGGTTCTCGGATGAGCGACTCAAACAGATTTACCAGTTTGAGTACGAGGGGCTCCTGCGCC GCAGTGCCCTTGCTACCAAAGACCGCTGCATCACCCTGGAAAAGTCCACCCAGAGCGTGCAGGGCCCGCGCTCAGCC GCCTGTGGACTTTTTTGCTGTATGTTCCTTCATGCCTTTGTGCACTGGCCCGACCGCCCCATGAACGGAAACCCCAC CATGAAGTTGCTGACTGGGGTGTCAAACAGCATGCTCCAATCACCCCAAGTCCAGCCCACCCTGCGTCGCAACCAGG AGGCGCTATATCGCTTCCTAAACACCCACTCATCTTACTTTCGTTCTCACCGCGCACGCATCGAAAGGGCCACCGCG TTTGACCGTATGGATATGCAATAAGTCATGTAAAACCGTGTTCAATAAAAAGCACTTTATTTTTACATGCACTAAGG CTCTGGTTTTTTGCTCATTCGTTTTCATCATTCACTCAGAAATCAAATGGGTTCTGGCGTGAGTCAGAGTGACCCGT GGGCAGGGAGACGTTGCGGAACTGTAACCTGTTCTGCCACTTGAACTCGGGGATCACCAGCTTGGGAACTGGAATTT CGGGAAAGGTGTCTTGCCACAACTTTCTGGTCAGTTGCAGGGCGCCAAGCAGGTCAGGAGCAGAGATCTTGAAATCA CAGTTGGGGCCGGCATTCTGGACACGGGAGTTGCGGTACACTGGGTTGCAACACTGGAACACCATCAAGGCTGGGTG TCTCACGCTTGCCAGCACGGTCGGGTCACTGATGGTAGTCACATCCAAGTCTTCAGCATTGGCCATTCCAAAGGGGG TCATCTTACAGGTCTGCCTGCCCATCACGGGAGCGCAGCCTGGCTTGTGGTTGCAATCGCAATGAATGGGGATCAGC ATCATCCTGGCTTGGTCGGGGGTTATCCCTGGGTACACGGCCTTCATGAAGGCTTCGTACTGCTTGAAAGCTTCCTG AGCCTTACTTCCCTCGGTGTAAAACATCCCACAGGACTTGCTGGAAAATTGGTTAGTAGCACAGTTGGCATCATTCA CACAGCAGCGGGCATCGTTGTTGGCCAACTGGACCACATTTCTGCCCCAGCGGTTCTGGGTGATCTTGGCTCTGTCT GGGTTCTCCTTCATAGCGCGCTGCCCGTTTTCGCTCGCCACATCCATCTCGATAATGTGGTCCTTCTGGATCATAAT AGTGCCATGCAGGCATTTCACCTTGCCTTCGTAATCGGTGCATCCATGAGCCCACAGAGCGCACCCGGTGCACTCCC AATTATTGTGGGCGATCTCAGAATAAGAATGCACCAATCCCTGCATGAATCTTCCCATCATCGCTGTCAGGGTCTTC ATGCTACTAAATGTCAGCGGGATGCCACGGTGCTCCTCGTTCACATACTGGTGGCAGATACGCTTGTACTGCTCGTG CTGCTCTGGCATCAGCTTGAAAGAGGTTCTCAGGTCATTATCCAGCCTATACCTCTCCATTAGCACAGCCATCACTT CCATGCCCTTCTCCCAGGCAGATACCAGGGGCAAGCTCAAAGGATTCCTAACAGCAATAGAAGTAGCTCCTTTAGCT ATAGGGTCATTCTTGTCGATCTTCTCAACACTTCTCTTGCCATCCTTCTCAATGATGCGCACCGGGGGGTAGCTGAA GCCCACGGCCACCAACTGAGCCTGTTCTCTTTCTTCTTCGCTGTCCTGGCTGATGTCTTGCAGAGGGACATGCTTGG TCTTCCTGGGCTTCTTCTTGGGAGGGATCGGGGGAGGACTGTTGCTCCGTTCCGGAGACAGGGATGACCGCGAAGTT TCGCTTACCAGTACCACCTGGCTCTCGATAGAAGAATCGGACCCCACGCGACGGTAGGTGTTCCTCTTCGGGGGCAG AGGTGGAGGCGACTGAGATGGGCTGCGGTCCGGCCTTGGAGGCGGATGGCTGGCAGAGCCCATTCCGCGTTCGGGGG TGTGCTCCCGTTGGCGGTCGCTTGACTGATTTCCTCCGCGGCTGGCCATTGTGTTCTCCTAGGCAGAGAAACAACAG ACATGGAAACTCAGCCATCACTGCCAACATCGCTGCAAGCGCCATCACACCTCGCCCCCAGCAGCGACGAGGAGGAG AGCTTAACCACCCCACCACCCAGTCCCGCTACCACCACCTCTACCCTCGATGATGAGGAGGAGGTCGACGCAGCCCA GGAGATGCAGGCGCAGGATAATGTGAAAGCGGAAGAGATTGAGGCAGATGTCGAGCAGGACCCGGGCTATGTGACAC CGGCGGAGCACGAGGAGGAGCTGAAACGTTTTCTAGACAGAGAGGATGACGACCGCCCAGAGCATCACCAGGAGGCT GGCCTCGGGGATCATGTTGCCGACTACCTCTCCGGGCTTGGGGGGGAGGACGTGCTCCTCAAACATCTAGCAAGGCA GTCGATCATAGTTAAAGACGCACTACTCAACCTCACCGAAGTGCCCATCAGTGTGGAAGAGCTTAGCCGCGCCTACG AGCTGAACCTCTTTTCGCCTCAGATACCCCCCAAGCGGCAGCGAAACGGCACCTGCGAGGCCAACCCTCGACTCAAC TTCTATCCAGCTTTTACTGTCCCCGAAGTGCTGGCCACCTACCACATCTTTTTTAAGAACCAAAAGATTCCAGTCTC CTGCCGCGCCAACCGCACCCGCGCAGATGCCCTTCTCAACTTGGGTCCGGGAGCTCGTTTACCTGATATAGCTTCCT TGGAAGAGGTTCCAAAGATCTTTGAGGGTCTGGGAAGTGATGAGACTCGGGCCGCAAATGCTCTGCAACAGGGAGAG AATGGCATGGATGAACATCACAGCGCTCTAGTGGAACTGGAGGGTGACAATGCCCGGCTTGCAGTGCTCAAGCGCAG TATCGTGGTCACCCATTTTGCCTACCCCGCTGTTAACCTGCCGCCCAAAGTCATGAGCGCTGTCATGGACCATCTGC TCATCAAACGAGCAAGTCCACTTTCAGAAAACCAGAACATGCAGGATCCAGACGCCTCGGACGAGGGCAAGCCGGTA GTCAGTGACGAGCAGCTATCTCGCTGGCTGGGTACCAACTCCCCCCGAGATTTGGAAGAAAGACGCAAGCTTATGAT GGCTGTAGTGCTAGTAACTGTTGAGTTGGAGTGTCTGCGCCGCTTTTTTACCGACCCCGAGACCCTGCGCAAGCTAG AGGAGAACCTGCACTACACCTTCAGACATGGCTTCGTGCGCCAGGCATGCAAGATCTCCAACGTGGAGCTCACCAAC CTGGTTTCATACATGGGCATTTTGCATGAGAACCGGCTAGGGCAGAGCGTTCTGCACACCACCCTGAAGGGGGAGGC CCGCCGCGACTACATCCGAGACTGTGTCTACCTCTACCTCTGCCATACCTGGCAGACTGGTATGGGTGTGTGGCAAC AGTGTTTGGAAGAGCAGAACCTTAAAGAGCTGGACAAGCTCTTGCAGAGATCCCTCAAAGCCCTGTGGACAGGTTTT GACGAGCGCACCGTCGCCTCGGACCTGGCGGACATCATCTTCCCCGAGCGTCTTAGGGTTACTCTGCGAAACGGCCT GCCAGACTTCATGAGCCAGAGCATGCTTAACAACTTTCGCTCTTTCATCCTGGAACGCTCCGGTATCCTGCCTGCCA CCTGCTGTGCGCTGCCCTCCGACTTTGTGCCTCTCACCTACCGCGAGTGCCCACCGCCGCTATGGAGCCACTGCTAC CTATTCCGCCTGGCCAACTACCTCTCCTACCACTCGGATGTGATAGAGGATGTGAGCGGAGACGGCCTGCTGGAATG CCACTGCCGATGCAATTTATGCACACCCCACCGCTCCCTCGCCTGCAACCCCCAGTTGCTAAGCGAGACCCAGATCA TCGGCACCTTCGAGTTGCAGGGTCCCAACAGTGAAGGCGAGGGGTCTTCTCCGGGGCAGAGTCTGAAACTGACACCG GGGCTGTGGACCTCCGCCTACCTGCGCAAGTTTTATCCCGAGGACTATCATCCCTATGAGATCAGGTTCTATGAGGA CCAGTCACATCCTCCCAAAGTCGAGCTCTCAGCCTGCGTCATCACCCAGGGGGCAATTCTGGCCCAATTGCAAGCCA TCCAAAAATCCCGCCAAGAATTTCTGCTGAAAAAGGGAAGCGGGGTCTACCTTGACCCCCAGACCGGTGAGGAGCTC AACACAAGGTTCCCCCAGGATGTCCCATCGCCGAGGAAGCAAGAAGCTGAAGGTGCAGCTGTCACCCCCAGAGGATA TGGAGGAAGACTGGGACAGTCAGGCAGAGGAGGAGATGGAAGATTGGGACAGCCAGGCAGAGGAGGTGGACAGCCTG GAGGAAGACAGTTTGGAGGAGGAAGACGAGGAGGCAGAGGAGGTGGAAGAAGCAACCGCCGCCAAACAGTTGTCATC GGCGGCGGAGACAAGCAAGTCCCCAGACAGCAGCACGGCTACCATCTCCGCTCCGGGTCGGGGGGCCCAGCGGCGGC CCAACAGTAGATGGGACGAGACCGGGCGATTTCCAAACCCGACCACCGCTTCCAAGACCGGTAAGAAGGAGCGACAG GGATACAAGTCCTGGCGTGGACATAAAAACGCTATCATCTCCTGCTTGCATGAATGCGGGGGCAACATATCCTTCAC CCGGCGATACCTGCTTTTCCACCACGGTGTGAACTTCCCCCGCAATATCTTGCATTACTACCGTCACCTCCACAGCC CCTACTGCAGTCAGCAAGTCCCGGCAACCCCGACAGAAAAAGACAGCAGCGACAACGGTGACCAGAAAACCAGCAGT TAGAAAATCCACAACAAGTGCAGCAGGAGGAGGACTGAGGATCACAGCGAACGAGCCAGCGCAGACCAGAGAGCTGA GGAACCGGATCTTTCCAACCCTCTATGCCATCTTCCAGCAGAGTCGGGGGCAAGAGCAGGAATTGAAAGTAAAAAAC CGATCTCTGCGCTCGCTCACCAGAAGTTGTTTGTATCACAAGAGCGAAGACCAACTTCAGCGCACTCTCGAGGACGC CGAGGCTCTCTTCAACAAGTACTGCGCGCTGACTCTTAAAGAGTAGCCCTTGCCCGCGCTCATTCGAAAACGGCGGG AATCACGTCACCCTTGGCAGCTGTCCTTTGCCCTCGTCATGAGTAAAGACATTCCCACGCCTTACATGTGGAGCTAT CAGCCCCAAATGGGGTTGGCAGCAGGTGCTTCCCAGGACTACTCCACCCGCATGAATTGGCTTAGCGCCGGGCCCTC AATGATATCACGGGTTAATGATATACGAGCTTATCGAAACCAGTTACTCCTAGAACAGTCAGCTCTTACCACCACAC CCCGCCAACACCTTAATCCCCGAAATTGGCCCGCCGCCCTGGTGTACCAGGAAAATCCCGCTCCCACCACCGTACTA CTTCCTCGAGACGCCCAGGCCGAAGTTCAGATGACTAACGCAGGTGTACAGCTGGCGGGCGGTTCCGCCCTATGTCG TCACCGGCCTCAACAGAGTATAAAACGCCTGGTGATCAGAGGCCGAGGTATCCAGCTCAACGACGAGTCGGTTAGCT CTTCGCTTGGTCTGCGACCAGACGGAGTCTTCCAGATCGCCGGCTGTGGGAGATCTTCCTTCACTCCTCGTCAGGCT GTGCTGACTTTGGAGAGTTCGTCCTCGCAGCCCCGCTCGGGCGGCATCGGAACTCTCCAGTTTGTGGAGGAGTTTAC TCCCTCTGTCTACTTCAACCCCTTCTCCGGCTCTCCTGGCCAGTACCCGGACGAGTTCATACCGAACTTCGACGCAA TCAGCGAGTCAGTGGATGGCTATGATTGATGTCTAATGGTGGCGCGGCTGAGCTAGCTCGACTGCGACACCTAGACC ACTGCCGCCGCTTTCGCTGTTTCGCCCGGGAACTCACCGAGTTCATTTACTTCGAACTCTCCGAGGAGCACCCTCAG GGTCCGGCCCACGGAGTGCGGATTACCATCGAAGGGGGAATAGACTCTCGCCTGCATCGCATCTTCTCCCAGCGGCC CGTGCTGATTGAGCGCGACCAGGGAAATACAACCATCTCCATCTACTGCATCTGTAACCACCCCGGATTGCATGAAA GCCTTTGCTGTCTTGTTTGTGCTGAGTTTAATAAAAACTGAGTTAAGACCCTCCTACGGACTACCGCTTCTTCAATC AGGACTTTACAACACCAACCAGATCTTCCAGAAGACCCAGACCCTTCCTCCTCTGATCCAGGACTCTAACTCTACCT TACCAGCACCCTCCACTACTAACCTTCCCGAAACTAACAAGCTTGGATCTCATCTGCAACACCGCCTTTCACGAAGC CTTCTTTCTGCCAATACTACCACTCCCAAAACCGGAGGTGAGCTCCGCGGTCTTCCTACTGACGACCCCTGGGTGGT AGCGGGTTTTGTAACGTTAGGATTAGTTGCGGGTGGGCTTGTGCTAATCCTTTGCTACCTATACACACCTTGCTGTG CATATTTAGTCATATTGTGCTGTTGGTTTAAGAAATGGGGGCCATACTAGTCGTGCTTGCTTTACTTTCGCTTTTGG GTCTGGGCTCTGCTAATCTCAATCCTCTTGATCACGATCCATGTCTAGACTTCGACCCAGAAAATTGCACACTTACT TTTGCACCCGACACAAGCCGTCTCTGTGGAGTTCTTATTAAGTGCGGATGGGACTGCAGGTCCGTTGAAATTACACA TAATAACAAAACATGGAACAATACATTATCCACCACATGGGAACCAGGAGTTCCCGAGTGGTATACTGTCTCTGTCC GAGGTCCTGACGGTTCCATTCGCATTAGTAACAACACTTTCATTTTTTCTGAAATGTGCGATCTGGCCATGTTTATG AGCAAACAGTATGACCTATGGCCTCCTAGCAAAGAGAACATTGTGGCATTTTCCATTGCTTATTGCTTGGTAACATG CATCATCACTGCTATCATTTGTGTGTGCATACACTTGCTTATAGTTATTCGCCCTAGACAAAGCAATGAGGAAAAAG AGAAAATGCCTTAACCTTTTTCCTCATACCTTTTCTTTACAGCATGGCTTCTGTTACAGCTCTAATTATTGCCAGCA TTGTCACTGTCGCTCACGGGCAAACAATTGTCCATATTACCTTAGGACATAATCACACTCTTGTAGGGCCCCCAATT ACTTCAGAGGTTATTTGGACCAAACTTGGAAGTGTTGATTATTTTGATATAATTTGCAACAAAACTGAACCAATATT TGTAATCTGTAACAGACAAAATCTCACGTTAATTAATGTTAGCAAAATTTATAACGGTTACTATTATGGTTATGATA GATCCAGTAGTCAATATAAAAATTACTTAGTTCGCATAACTCAGCCCAAATCAACAGTGCCAACTATGACAATAATT AAAATGGCTAATAAAGCATTAGAAAATTTTACATTACCAACAACGCCCAATGAAAAAAACATTCCAAATTCAATGAT TGCAATTATTGCGGCGGTGGCATTGGGAATGGCACTAATAATAATATGCATGTTCCTATATGCTTGTTGCTATAAAA AGTTTCAACATAAACAGGATCCACTACTAAATTTTAACATTTAATTTTTTATACAGATGATTTCCACTACAATTTTT ATCATTACTAGCCTTGCAGCTGTAACTTATGGCCGTTCACACCTAACTGTACCTGTTGGCTCAACATGTACACTACA AGGACCCCAAGAAGGCTATGTCACTTGGTGGAGAATATATGATAATGGAGGGTTCGCTAGACCATGTGATCAGCCTG GTACAAAATTTTCATGCAACGGAAGAGACTTGACCATTATTAACATAACATTAAATGAGCAAGGCTTCTATTATGGA ACCAACTATAAAAATAGTTTAGATTACAACATTATTGTAGTGCCAGCCACCACTTCTGCTCCCCGCAAATCCACTTT CTCTAGCAGCAGTGCCAAAGCAAGCACAATTCCTAAAACAGCTTCTGCTATGTTAAAGCTTCGAAAAATCGCTTTAA GTAATTCCACAGCAGCTCCCAATACAATTCCTAAATCAACAATTGGCATCATTACTGCCGTGGTAGTGGGATTAATG ATTATATTTTTGTGCATAATGTACTACGCCTGCTGCTATAGAAAACATGAACAAAAAGGTGATGCATTACTAAATTT TGATATTGTTTCAATCAAATGCCACTAACACTCTCAATGTGCAGACTACTTTAAAACATGACATGGAAAACCACACT ACCTCCTATGCATACACAAATATTCAGCCTAAATACGCTATGCAACTTAGAAATCACCATACTAATTGTAATTGGAA TTCTTACACTATCTGTTATTCTTTATTTTATATTCTGCCGTCAAATACCCAATGTTCATAGAAATTCTAAAAGACGT CCCATCTATTCTCCTATGATTAGTCGTCCCCATATGGCTCTGAATGAAATCTAAGATCTTTTTTTTTCTTTTACAGT ATGGTGAACATCAATCATGATTCCTAGAAATTTCTTCTTCACCATACTCATCTGTGCTTTCAATGTCTGTGCTACTT TCACAGCAGTAGCCACTGCAAGCCCAGACTGTATAGGACCATTTGCTTCCTATGCACTTTTTGCCTTTGTTACTTGC ATCTGCGTGTGTAGCATAGTCTGCCTGGTTATTAATTTTTTCCAACTGGTAGACTGGATCTTTGTGCGAATTGCCTA CCTACGTCACCATCCCGAATACCGCAATCAAAATGTTGCGGCACTTCTTAGGCTTATTTAAAACCATGCAGGCTATG CTACCAGTTATTTTAATTCTGCTACTACCCTGCATTGCCCTACCTTCCACCGCCACTCGCGCTACACCTGAACAACT TAGAAAATGCAAATTTCAACAACCATGGTCATTTCTTGATTGCTACCATGAAAAATCTGATTTTCCCACATACTGGA TAGTGATTGTTGGAATAATTAACATACTTTCATGTACCGTTTTCTCAATCACAATATACCCCACATTTAATTTTGGG TGGAATTCTCCCAATGCACTGGGTTACCCACAAGAACTAGATGAACATATCCCACTACAACACATACAACAACCACT AGCATTGGTAGAGTATGAAAATGAGCCACAACCTTCACTGCCTCCTGCTATTAGTTACTTCAACCTAACCGGCGGAG ATGACTGAAATACTCACCACCTCCAATTCCGCCGAGGATCTGCTTGATATGGACGGCCGCGCCTCAGAACAGCGACT CGCCCAACTACGCATCCGCCAGCAGCAGGAACGCGTGACCAAAGAGCTCAGAGATGTCATCCAAATTCACCAATGCA AAAAAGGCATATTTTGTTTGGTAAAACAAGCCAAGATATCCTACGAGATCACCGCTACTGACCATCGCCTTTCTTAC GAACTTGGCCCCCAACGACAAAAATTTACATGCATGGTGGGAATCAACCCTATAGTTATCACCCAGCAAAGTGGAGA TACTAAGGGTTGCATTCACTGCTCTTGCGATTCCACCGAGTGCACCTACACCCTGCTGAAGACCCTATGCGGCCTAA GAGACCTGCTACCCATGAATTAAAAATTAATAAAAAATTACTTACTTGAAATCAGCAATAAGGTCTCTGTTGAAATT TTTTCCCAGCAGCACCTCGCTTCCCTCTTCCCAACTCTGGTATTCTAAACCCCGTTCAGCGGCATACTTTCTCCATA CTTTAAATGGGATGTCAAATTTTAGCTCCTCTCCTGTACCCACGATCTTCATGTCTTTCTTCCCAGATGACCAAGAG AGTCCGGCTCAGTGATTCCTTCAACCCTGTCTACCCCTATGAAGATGAAAGCACCTCCCAACACCCCTTTATAAACC CAGGGTTTATTTCCCCAAATGGCTTTACACAAAGCCCAGACGGAGTTCTTACTTTAAATTGTTTAACCCCACTAACA ACCACAGGCGGGCCTTTACAGTTAAAAGTGGGAGGGGGACTTATAGTGGATGACACTGATGGGACCTTACAAGAAAA CATACGTGCTACAGCACCCATTACTAAAAATAATCATTCTGTAGAACTATCCATTGGAAATGGATTAGAAACACAAA ACAATAAACTATGTGCCAAATTGGGAAATGGGTTAAAATTTAACAACGGTGACATTTGTATAAAGGATAGTATTAAC ACCTTATGGACTGGAATAAAGCCTCCACCTAACTGTCAAATTGTGGAAAACACTGATACAAACGATGGCAAACTTAC TTTAGTATTAGTAAAAAACGGAGGGCTTGTTAATGGCTACGTATCTCTAGTTGGTGTATCAGACACTGTGAACCAAA TGTTCACACAAAAGTCAGCAACCATACAATTAAGATTATATTTCGACTCTTCTGGAAATCTATTAACTGATGAATCA AACTTAAAAATTCCACTTAAAAATAAATCTTCTACAGCAACCAGTGAAGCTGCAACCAGCAGCAAAGCCTTTATGCC AAGTACTACAGCTTATCCCTTTAACACCACTACTAGGGATAGTGAAAACTATATTCATGGAATATGTTACTATATGA CTAGTTATGATAGAAGTCTAGTTCCCTTAAACATTTCTATAATGCTAAACAGCCGTACGATTTCTTCCAATGTTGCC TATGCCATACAATTTGAATGGAATCTAAATGCAAAAGAATCTCCAGAAAGCAACATAGCTACGCTGACCACATCCCC CTTTTTCTTTTCTTATATTAGAGAAGACGACAACTAAAAAATAAAGTTTAAGTGTTTTTATTTAAAAATCACAAAAT TCGAGTAGTTATTTTGCCTCCCCCTTCCCATTTAACAGAATACACCAATCTCTCCCCACGCACAGCTTTAAACATTT GGATACCATTAGAGATAGACATAGTTTTAGTTTCCACATTCCAAACAGTTTCAGAGCGAGCCAATCTGGGGTCAGTG ATACATAAAAATGCATCGGGATAGTCTTTTAAAGCGCTTTCACAGTCCAACTGCTGCGGATGCGACTCCGGAGTCTG GATCACAGTCATCTGGAAGAAGAACGATGGGAATCATAATCCGAAAACGGAATCGGGCGATTGTGTCTCATCAAACC CACAAGCAGCCGCTGTCTGCGTCGCTCCGTGCGACTGCTGTTTATGGGATCGGGGTCTGCAGTGTCCTGAAGCATGA TTTTAATAGCCCTTAACATTAACTTTCTGGTGCGATGCGCGCAGCAACGCATTCTGATTTCACTTAGATTACTACAG TATGTACAGCACATTATCACAATATTGTTTAATAAACCATAATTAAAAGCGCTCCAGCCAAAACTCATATCTGATAC AATCGCCCCTGCATGACCATCATACCAAATTTTAATATAAATTAAATGTCGTTCCCTCAAAAACACACTACCCACAT ACATGATCTCTTTTGGCATGTGCATATTAACAATCTGTCTGTACCATGGACAACGTTGGTTAATCATGCAACCCAAT ATAACCTTCCGGAACCACACTGCCAACACCGCTCCCCCAGCCATGCATTGAAGTGAACCCTGCTGATTACAATGACA ATGAAGAACCCAATTCTCTCGACCATGAATCACTTGAGACTGAAAAATATCTATAGTAGCACAACAAAGACATAAAT GCATGCATCTTCTCATAATTTTTAACTCATCTGGATTTAAAAACATATCCCAAGGAATGGGAAACTCTTGCAAAACA GTAAAGCTGGCAGAACAAGGAAGACCACGAACACAACTTACACTATGCATAGTCATAGTATCACAATCTGGCAACAG CGGGTGGTCTTCAGTCATAGAAGCTCGGGTTTCATTTTCCTCACATCGTGGTAACTGGGCTCTGGTGTAAGGGTGAT GTCTGGCGCATGATGTCGAGCGTGCGCGCAACCTTGTCATAATGGAGTTGTTTCCTGACATTCTCGTATTTTGTATA GCAAAATGCGGCCCTGGCACAACACACTCTTCTTCGTCTTCTATCCTGCCGCTTAGTGTGTTCCGTCTGATAATTCA AGTACAGCCACACTCTTAAGTTGGTCAAAAGAATGCTGGCTTCAGTTGTAATCAAAACTCCATCATATTTAATTGTT CTAAGGAAATCATCCACGGTAGCATATGCAAATCCCAACCAAGCAATGCAACTGGATTGTGTTTCAAGCAGCAGAGG AGAGGGAAGAGACGGAAGAATCATGTTAATTTTTATTCCAAACGATCTCGCAGTACTTCAAATTGTAGATCGCGCAG ATGGCATCTATCGCCCCCACTGTGTTGGTGAAAAAGCACAGCTAAATCAAAAGAAATGCGATTTTCAAGGTGCTCAA CGGTGGCTTCCAACAAAGCCTCCACGCGCACATCCAAAAACAAAAGAATACCAAAAGAAGGAGCATTTTCTAACTCC TCAAACATCATATTACATTCCTGCACCATTCCCAGATAATTTTCAGCTTTCCAGCCTTGAATTATTCGTGTCAGTTC TTGTGGTAAATCCAAACCACACATTACAAACAGGTCACGGAGGGCGCCCTCCACCACCATTCTTAAACACACCCTCA TAATGACAAAATATCTTGCTCCTGTGTCACCTGTAGCAAATTAAGAATGGCATCATCAATTGACATGCCCTTGGCTC TAAGTTCTTCTCTAAGTTCTAGTTGTAAATACTCTCTCATATTATCACCAAACTGCTTAGCCAAAAGCCCCCCGGGA ACAATAGCAGGGGACGCTACAGTGCAGTACAAGCGCAGACCTCCCCAATTGGCTCCAGCAAAAACAAGATTAGAATA AGCATACTGGGAACCACCAGTAATATCATCAAAGTTGCTGGAAATATAATCAGGCAGAGTTTCTTGTAAAAATTGAA TAAAAGAAAAATTTTCCAAAGAAACATTCAAAATCTCTGGGATGCAAATGCAATAGGTTACCGCGCTGCGCTCCAAC ATTGTTAGTTTTGAATTAGTCTGCAAAATAAAAGAAACAAGCGTCATATCATAGTAGCCTGTCGAACAGGTGGATAA ATCAGTCTTTCCATCACAAGACAAGCCACAGGGTCTCCAGCTCGACCCTCGTAAAACCTGTCATCGTGATTAAACAA CAGCACCGAAAGTTCCTCGCGGTGGCCAGCATGAATAATTCTTGATGAAGCATATAATCCAGACATGTTAGCATCAG TTAAAGAGAAAAAACAGCCAACATAGCCTCTGGGTATAATTATGCTTAATCTTAAGTATAGCAAAGCCACCCCTCGC GGATACAAAGTAAAAGGCACAGGAGAATAAAAAATATAATTATTTCTCTGCCGCTGTTCAGGCAACGTCGCCCCCGG TCCATCTAAATACACATACAAAGCCTCATCAGCCATGGCTTACCAGACAAAGAACAGCGGGCGCACAAAGCACAAGC TCTAAAGAAGCTCTAAAGACACTCTCCAACCTCTCCACAATATATACACAAGCCCTAAACTGACGTAATGGGAGTAA AGTATAAAAAATCCCGCCAAGCCCAACACACACCCCGAAACTGCGTCAGCAGGGAAAAATACAGTTTCACTTCCGCA TTCCCAACAAGCGTAAGTTCCTCTTTCTCATGGTACGTCACATCCGATTAACTTGCAACGTCATTTTCCCACGGTCG CACCGCCCCTTTTAGCCGTTCACCCCGCAGCCAATCACCACACAGCGCGCACTTTTTTAAATTACCTCATTTGCATA TTGGCACCATTCCATCTATAAGGTATATTATATAGATAG

GenBank Accession No. AAW33370 MTKRVRLSDSFNPVYPYEDESTSQHPFINPGFISPNGFTQSPDGVLTLNCLTPLTTTGGPLQLKVGGGLIVDDTDGT LQENIRATAPITKNNHSVELSIGNGLETQNNKLCAKLGNGLKFNNGDICIKDSINTLWTGIKPPPNCQIVENTDTND GKLTLVLVKNGGLVNGYVSLVGVSDTVNOMFTQKSATIQLRLYFDSSGNLLTDESNLKIPLKNKSSTATSEAATSSK AFMPSTTAYPFNTTTRDSENYIHGICYYMTSYDRSLVPLNISIMLNSRTISSNVAYAIQFEWNLNAKESPESNIATL TTSPFFFSYIREDDN

GenBank Accession No. AAW33349 MMRRTVLGGAVVYPEGPPPSYESVMQQAAAATMQPPLEAPFVPPRYLAPTEGRNSIRYSELAPLYDTTRLYLVDNKS ADIASLNYQNDHSNFLTTVVONNDFTPTEASTQTINFDERSRWGGOLKTIMHTNMPNVNEYMESNKFKARVMVSRKA PEGVTVDDNYDHKQDILEYEWFEFTLPEGNFSATMTIDLMNNAIIDNYLEVGRONGVLESDIGVKEDTRNFRLGWDP ETKLIMPGVYTYEAFHPDIVLLPGCGVDFTESRLSNLLGIRKRHPFQEGFKILYEDLEGGNIPALLDVEAYENSKKE QEAKTEAAKAAAIAKANIVVSDPVRVANAEEVRGDNYTASSVATDESLLAAVAETTETKLTIKPVEKDSKSRSYNVL EDKVNTAYRSWYLSYNYGDPEKGVRSWTLLTTSDVTCGAEQVYWSLPDMMQDPVTFRSTRQVSNYPVVGAELMPVFS KSFYNEQAVYSQQLRQSTSLTHVENRFPENQILIRPPAPTITTVSENVPALTDHGTLPLRSSIRGVQRVTVTDARRR TCPYVYKALGIVAPRVLSSRTF

GenBank Accession No. AAW33354 MATPSMLPQWAYMHIAGQDASEYLSPGLVQFARATDTYFNLGNKFRNPTVAPTHDVTTDRSQRLMLRFVPVDREDNT YAYKVRYTLAVGDNRVLDMASTFFDIRGVLDRGPSFKPYSGTAYNSLAPKGAPNTSQWIAEGVKKEDGGSDEEEEKN LTTYTFGNAPVKAEGGDITKDKGLPIGSEITDGEAKPIYADKLYQPEPQVGDETWTDTDGTTEKYGGRALKPETKMK PCYGSFAKPTNVKGGQAKOKTTEQPONQQVEYDIDMNFFDEASQKANFSPKIVMYAENVDLETPDTHVVYKPGTSEE SSHANLGQQSMPNRPNYIGFRDNFIGLMYYNSTGNMGVLAGQASQLNAVVDLQDRNTELSYQLLLDSLGDRTRYFSM WNQAVDSYDPDVRIIENHGVEDELPNYCFPLDGVGVPISSYKIIEPNGQGADWKEPDINGTSEIGOGNLFAMEINLQ ANLWRSFLYSNVALYLPDSYKYTPANVTLPTNTNTYDYMNGRVVPPSLVDTYVNIGARWSLDAMDNVNPFNHHRNAG LRYRSMLLGNGRYVPFHIQVPQKFFAVKNLLLLPGSYTYEWNFRKDVNMVLQSSLGNDLRVDGASISFTSINLYATF FPMAHNTASTLEAMLRNDTNDQSFNDYLSAANMLYPIPANATNVPISIPSRNWAAFRGWSFTRLKTKETPSLGSGFD PYFVYSGSIPYLDGTFYLNHTFKKVSIMFDSSVSWPGNDRLLSPNEFEIKRTVDGEGYNVAQCNMTKDWFLVQMLAN YNIGYQGFYVPEGYKDRMYSFFRNFQPMSRQVVDEINYKDYKAVAVPYQHNNSGFVGYMAPTMRQGQAYPANYPYPL IGTTAVTSVTQKKFLCDRTMWRIPFSSNFMSMGALTDLGQNLLYANSAHALDMTFEVDPMDEPTLLYLLFEVFDVVR VHQPHRGVIEAVYLRTPFSAGNATT

GenBank Accession No. AY737797 (SEQ ID NO: 215) CATCATCAATAATATACCTTATAGATGGAATGGTGCCAATATGTAAATGAGGTGATTTTAAAAATTGTGGGGTGTGT GGTGATTGGCTGTGGGGTTAACGGCTAAACGGGGCGGCGCGGCCGTGGGAAAATGACGTTTTGTGGGGGTGGAGTTT TTTTGCAAGTTGTCGCGGGAAATGTGACGCATAAAAAGGCTTTTTTTCTCACGGAACTACTGACTTTTCCCACGGTA TTTAACAGGAAATGAGGTAGTTTTGACCGGATGCAAGTGAAAATTGCTGATTTGCGCGCGAAAACTGAATGAGGAAG TGTTTTTCTGAATAATGTGGTATTTATGGCAGGGTGGAGTATTTGTTCAGGGCCAGGTAGACTTTGACCCATTACGT GGAGGTTTCGATTACCGTGTTTTTTACCTGAATTTCCGCGTACCGTGTCAAAGTCTTCTGTTTTTACGTAGGTGTCA GCTGATCGCTACGGTATTTATACCTCAGGGTTTGTGTCAAGAGGCCACTCTTGAGTGCCAGCGAGAAGAGTTTTCTC CTCTGCGCCGGCAGTTTAATAATAAAAAAATGAGAGATTTGCGATTTCTGCCTCAGGAAATAATTTCTGCTGAGACT GGAAATGAAATACTGGAGCTTGTGGTGCACGCCCTGATGGGAGACGATCCGGAGCCACCTGTGCAGCTTTTTGAGCC TCCTACGCTTCAGGAACTGTATGATTTAGAGGTAGAGGGATCGGAGGATTCTAATGAGGAAGCTGTGAATGGCTTTT TTACCGATTCTATGCTTTTAGCTGCTAATGAAGGATTAGAATTAGATCCGCCTTTGGACACTTTCGATACTCCAGGG GTGATTGTGGAAAGCGGTACAGGTGTAAGAAAATTACCTGATTTGGGTTCCGTGGACTGTGATTTGCACTGCTATGA AGACGGGTTTCCTCCGAGTGATGAGGAGGACCATGAAAAGGAGCAGTCTATGCAGACTGCAGCGGGTGAGGGAGTGA AGGCTGCCAGTGTTGGTTTTCAGTTGGATTGCCCGGAGCTTCCTGGACATGGCTGTAAGTCTTGTGAATTTCACAGG AAAAATACTGGAGTAAAGGAACTGTTATGTTCGCTTTGTTATATGAGAGCGCACTGCCACTTTATTTACAGTAAGTG TGTTTAAGTTAAAATTTAAAGGAATATGCTGTTTTTCACATGTATATTGAGTGGGAGTTTTGTGCTTCTTATTATAG GTCCTGTGTCTGATGCTGATGAGTCACCATCTCCTGATTCTACTACCTCACCTCCTGAGATTCAAGCACCTGTTCCT GTGGACGTGCGCAAGCCCATTCCTGTGAAGCTTAAGCCTGGGAAACGTCCAGCAGTGGAAAAACTTGAGGACTTGTT ACAGGGTGGGGACGGACCTTTGGACTTGAGTACACGGAAACGGCCAAGACAATAAGTGTTCCATATCCGTGTTTACT TAAGGTGACGTCAATATTTGTGTGAGAGTGCAATGTAATAAAAATATGTTAACTGTTCACTGGTTTTTATTGCTTTT TGGGCGGGGACTCAGGTATATAAGTAGAAGCAGACCTGTATGGTTAGCTCATAGGAGCTGGCTTTCATCCATGGAGG TTTGGGCCATTTTGGAAGACCTTAGAAAGACTAGGCAACTGTTAGAGGACGCTTCGGACGGAGTCTCCGGTTTTTGG AGATTCTGGTTCGCTAGTGAATTAGCTAGGGTAGTTTTTAGGATAAAACAGGACTATAAAGAAGAATTTGAAAAGTT GTTGGTAGATTGCCCAGGACTTTTTGAAGCTCTTAATTTGGGCCATCAAGTTCACTTTAAAGAAAAAGTTTTATCAG TTTTAGACTTTTCAACCCCAGGTAGAACTGCCGCTGCTGTGGCTTTTCTTACTTTTATATTAGATAAATGGATCCCG CAGACTCATTTCAGCAGGGGATACGTTTTGGATTTCGTAGCCACAGCATTGTGGAGAACATGGAAGGTTCGCAAGAT GAGGACAATCTTAGGTTACTGGCCAGTGCAGCCTTTGGGTGTAGCGGGAATCCTGAGGCATCCACCGGTCATGCCAG CGGTTCTGGAGGAGGAACAGCAAGAGGACAACCCGAGAGCCGGCCTGGACCCTCCAGTGGAGGAGGCGGAGTAGCTG ACTTGTCTCCTGAACTGCAACGGGTGCTTACTGGATCTACGTCCACTGGACGGGATAGGGGCGTTAAGAGGGAGAGG GCATCTAGTGGTACTGATGCTAGATCTGAGTTGGCTTTAAGTTTAATGAGTCGCAGACGTCCTGAAACCATTTGGTG GCATGAGGTCCAGAAAGAGGGAAGGGATGAAGTTTCTGTATTGCAGGAGAAATATTCACTGGAACAGGTGAAAACAT GTTGGTTGGAGCCTGAGGATGATTGGGAGGTGGCCATTAAAAATTATGCCAAGATAGCTTTGAGGCCTGATAAACAG TATAAGATTACTAGACGGATTAATATCCGGAATGCTTGTTACATATCTGGAAATGGGGCTGAGGTGGTAATAGATAC TCAAGACAAGGCAGTTATTAGATGCTGCATGATGGATATGTGGCCTGGAGTAGTCGGTATGGAAGCAGTAACTTTTG TAAATGTTAAGTTTAGGGGAGATGGTTATAATGGAATAGTGTTTATGGCCAATACCAAACTTATATTGCATGGTTGT AGCTTTTTTGGTTTCAACAATACCTGTGTAGATGCCTGGGGACAGGTTAGTGTACGGGGATGTAGTTTCTATGCGTG TTGGATTGCCACAGCTGGCAGAACCAAGAGTCAATTGTCTCTGAAGAAATGCATATTCCAAAGATGTAACCTGGGCA TTCTGAATGAAGGCGAAGCAAGGGTCCGCCACTGCGCTTCTACAGATACTGGATGTTTTATTTTAATTAAGGGCAAT GCCAGCGTAAAGCATAACATGATTTGCGGTGCTTCCGATGAGAGGCCTTATCAAATGCTCACTTGTGCCGGTGGGCA TTGTAATATGCTGGCTACTGTGCATATTGTTTCCCATCAACGCAAAAAATGGCCTGTTTTTGATCACAATGTGTTGA CCAAGTGTACCATGCATGCAGGTGGGCGTAGAGGAATGTTTATGCCTTACCAGTGTAACATGAATCATGTGAAAGTG TTGTTGGAACCAGATGCCTTTTCCAGAATGAGCCTAACAGGAATCTTTGACATGAACATGCAAATCTGGAAGATCCT GAGGTATGATGATACGAGATCGAGGGTGCGCGCATGCGAATGCGGAGGCAAGCATGCCAGGTTCCAGCCGGTGTGTG TAGATGTGACTGAAGATCTGAGACCGGATCATTTGGTTATTGCCCGCACTGGAGCAGAGTTCGGATCCAGTGGAGAA GAAACTGACTAAGGTGAGTATTGGGAAAACTTGGGGTGGGGTTTTCAGATGGACAGATTGAGTAAAAATTTGTTTTT TCTGTCTTTCAGCTGTCATGAGTGGAAACGCTTCTTTTAAGGGGGGAGTCTTCAGCCCTTATCTGACAGGGCGTCTC CCATCCTGGGCAGGAGTTCGTCAGAATGTTATGGGATCTACTGTGGATGGAAGACCCGTCCAACCCGCCAATTCTTC AACGCTGACCTATGCTACTTTAAGTTCTTCACCTTTGGACGCAGCTGCAGCCGCCGCCGCCGCCTCTGTTGCCGCTA ACACTGTGCTTGGAATGGGTTACTATGGAAGTATCGTGGCTAATTCCACTTCCTCTAATAACCCTTCTACCCTGACT CAGGACAAGTTACTTGTCCTTTTGGCCCAGCTGGAGGCTTTGACCCAACGTCTGGGTGAACTTTATCAGCAGGTGGC CGAGTTGCGAGTACAAACTGAGTCTGCTGTCGGCACGGCAAAGTCTAAATAAAAAAAAATTCCACAATCAATGAATA AATAAACGAGCTTGTTGTTGATTTAAAATCAAGTGTTTTTATTTCATTTTTCGCGCACGGTATGCCCTAGACCACCG ATCTCGATCATTGAGAACACGGTGGATTTTTTCCAGAATCCTATAGAGGTGGGATTGAATGTTTAGATACATGGGCA TTAGGCCATCTTTGGGGTGGAGATAGCTCCATTGAAGGGATTCATGCTCCGGGGTAGTGTTGTAAATCACCCAGTCA TAACAAGGTCGCAGTGCATGGTGTTGCACAATATCTTTTAGAAGTAGGCTGATTGCCACAGATAAGCCCTTGGTGTA GGTGTTTACAAACCGGTTGAGCTGGGAGGGGTGCATTCGGGGTGAAATTATGTGCATTTTGGATTGGATTTTTAAGT TGGCAATATTGCCGCCAAGATCTCGTCTTGGGTTCATGTTATGAAGGACCACCAAGACGGTGTATCCGGTACATTTA GGAAATTTATCGTGTAGCTTGGATGGAAAAGCGTGGAAAAATTTGGAGACACCCTTGTGTCCTCCGAGATTTTCCAT GCACTCATCCATGATAATAGCAATGGGGCCGTGGGCAGCAGCGCGGGCAAACACGTTCCGTGGGTCTGACACATCAT AGTTATGTTCCTGAGTTAAATCATCATAAGCCATTTTAATGAATTTGGGGCGGAGAGTACCCGATTGGGGTATGAAT GTTCCTTCGGGCCCCGGAGCATAGTTCCCCTCACAGATTTGCATTTCCCAAGCTTTCAGTTCCGATGGTGGAATCAT GTCCACCTGGGGGGCTATGAAGAACACCGTTTCTGGGGCGGGGGTGATTAGTTGGGATGATAGCAAGTTTCTGAGCA ATTGAGATTTGCCACATCCGGTGGGGCCATAAATGATTCCGATTACAGGTTGCAGGTGGTAGTTTAGGGAACGGCAA CTGCCGTCTTCTCGAAGCAAGGGGGCCACCTCGTTCATCATTTCCCTTACATGCATATTTTCCCGCACCAAATCCAT TAGGAGGCGCTCTCCTCCTAGTGATAGAAGTTCTTGTAGTGAGGAAAAGTTTTTCAGCGGTTTTAGACCGTCAGCCA TGGGCATTTTGGAGAGAGTTTGCTGCAAAAGTTCTAGTCTGTTCCACAGTTCAGTGATGTGTTCTATGGCATCTCGA TCCAGCAGACCTCCTCGTTTCGCGGGTTTGGACGGCTCCTGGAGTAGGGTATGAGACGATGGGCGTCCAGCGCTGCC AGGGTTCGGTCCTTCCAGGGTCTCAGTGTTCGAGTCAGGGTTGTTTCCGTCACAGTGAAGGGGTGTGCGCCTGCTTG GGCGCTTGCCAGGGTGCGCTTCAGACTCATTCTGCTGGTGGAGAACTTCTGTCGCTTGGCGCCCTGTATGTCGGCCA AGTAGCAGTTTACCATGAGTTCGTAGTTGAGCGCCTCGGCTGCGTGGCCTTTGGCGCGGAGCTTACCTTTGGAAGTT TTCTTGCATACCGGGCAGTATAGGCATTTCAGCGCATACAGCTTGGGCGCAAGGAAAATGGATTCTGGGGAGTATGC ATCTGCGCCGCAGGAGGCGCAAACAGTTTCACATTCCACCAGCCAGGTTAAATCCGGTTCATTGGGGTCAAAAACAA GTTTTCCGCCATATTTTTTGATGCGTTTCTTACCTTTGGTCTCCATGAGTTCGTGTCCTCGTTGAGTGACAAACAGG CTGTCCGTATCCCCGTAGACTGATTTTACAGGCCTCTTCTCCAGTGGAGTGCCTCGGTCTTCTTCGTACAGGAACTC TGACCACTCTGATACAAAGGCGCGCGTCCAGGCCAGCACAAAGGAGGCTATGTGGGAGGGGTAGCGATCGTTGTCAA CCAGGGGGTCCACCTTTTCCAAAGTATGCAAACACATGTCACCCTCTTCAACATCCAGGAATGTGATTGGCTTGTAG GTGTATTTCACGTGACCTGGGGTCCCCGCTGGGGGGGTATAAAAGGGGGCGGTTCTTTGCTCTTCCTCACTGTCTTC CGGATCGCTGTCCAGGAACGTCAGCTGTTGGGGTAGGTATTCCCTCTCGAAGGCGGGCATGACCTCTGCACTCAGGT TGTCAGTTTCTAAGAACGAGGAGGATTTGATATTGACAGTGCCGGTTGAGATGCCTTTCATGAGGTTTTCGTCCATT TGGTCAGAAAACACAATTTTTTTATTGTCAAGTTTGGTGGCAAATGATCCATACAGGGCGTTGGATAAAAGTTTGGC AATGGATCGCATGGTTTGGTTCTTTTCCTTGTCCGCGCGCTCTTTGGCAGCGATGTTGAGTTGGACATACTCGCGTG CTAGGCACTTCCATTCGGGGAAGATAGTTGTCAATTCATCTGGCACGATTCTCACTTGCCACCCTCGATTATGCAAG GTAATTAAATCCACACTGGTGGCCACCTCGCCTCGAAGGGGTTCGTTGGTCCAACAGAGCCTACCTCCTTTCCTAGA ACAGAAAGGGGGAAGTGGGTCTAGCATAAGTTCATCGGGAGGGTCTGCATCCATGGTAAAGATTCCCGGAAGTAAAT CCTTATCAAAATAGCTGATGGGAGTGGGGTCATCTAAGGCCATTTGCCATTCTCGAGCTGCCAGTGCACGCTCATAT GGGTTAAGGGGACTGCCCCAGGGCATGGGATGGGTGAGTGCAGAGGCATACATGCCACAGATGTCATAGACGTAGAT GGGATCCTCAAAGATGCCTATATAGGTTGGATAGCATCGCCCCCCTCTGATACTTGCTCGCACATAGTCATATAGTT CATGTGATGGCGCTAGCAACCCCGGACCCAAGTTGGTGCGATTGGGTTTTTCTGTTCTGTAGACAATCTGGCGAAAG ATGGCGTGAGAATTGGAAGAGATGGTGGGTCTTTGAAAAATGTTGAAATGGGCATGAGGTAGACCTACAGAGTCTCT GACAAAGTGGGCATAAGATTCTTGAAGCTTGGTTACCAGTTCGGCGGTGACAAGTACGTCTAGGGCGCAGTAGTCAA GTGTTTCTTGAATGATGTCATAACCTGGTTGGTTTTTCTTTTCCCACAGTTCGCGGTTGAGAAGGTATTCTTCGCGA TCCTTCCAGTACTCTTCTAGCGGAAACCCGTCTTTGTCTGCACGGTAAGATCCTAGCATGTAGAACTGATTAACTGC CTTGTAAGGGCAGCAGCCCTTCTCTACGGGTAGAGAGTATGCTTGAGCAGCTTTTCGCAGCGAAGCGTGAGTAAGGG CGAAGGTGTCTCTGACCATGACTTTGAGAAATTGGTATTTGAAGTCCATGTCGTCACAGGCTCCCTGTTCCCAGAGT TGGAAGTCTACCCGTTTCTTGTAGGCGGGGTTGGGCAAAGCGAAAGTAACATCGTTGAAGAGAATCTTACCGGCTCT GGGCATAAAATTGCGAGTGATGCGGAAAGGCTGTGGTACTTCCGCTCGATTGTTGATCACCTGGGCAGCTAGGACGA TCTCGTCGAAACCGTTGATGTTGTGTCCTACGATGTATAATTCTATGAAACGCGGCGTGCCTTTGACGTGAGGTAGC TTATTGAGCTCATCAAAGGTTAGGTCTGTAGGGTCAGATAAGGCGTAGTGTTCGAGAGCCCATTCGTGCAGGTGAGG ATTTGCATGTAGGAATGATGACCAAAGATCCACCGCCAGTGCTGTTTGTAACTGGTCCCGATACTGACGAAAATGCT GGCCAATTGCCATTTTTTCTGGAGTGACACAGTAGAAGGTTCTGGGGTCTTGTTGCCATCGATCCCACTTTAGTTTA ATGGCTAGATCGTGGGCCATGTTGACGAGACGCTCTTCTCCTGAGAGTTTCATGACCAGCATGAAAGGAACTAGTTG TTTGCCAAAGGACCCCATCCAGGTGTAAGTTTCCACATCGTAGGTCAGGAAGAGTCTTTCTGTGCGAGGATGAGAGC CGATCGGGAAGAACTGGATTTCCTGCCACCAGTTGGAGGATTGGCTGTTGATGTGATGGAAGTAGAAGTTTCTGCGG CGCGCCGAGCATTCGTGTTTGTGCTTGTACAGACGGCCGCAGTAGTCGCAGCGTTGCACGGGTTGTATCTCGTGAAT GAGCTGTACCTGGCTTCCCTTGACGAGAAATTTCAGTGGGAAGCCGAGGCCTGGCGATTGTATCTCGTGCTCTTCTA TATTCGCTGTATCGGCCTGTTCATCTTCTGTTTCGGTGGTGGTCATGCTGACGAGCCCCCGCGGGAGGCAAGTCCAG ACCTCGGCGCGGGAGGGGCGGAGCTGAAGGACCAGAGCGCGCAGGCTGGAGCTGTCCAGAGTCCTGAGACGCTGCGG ACTCAGGTTAGTAGGTAGGGACAGAAGATTAACTTGCATGATCTTTTCCAGGGCGTGCGGGAGGTTCAGATGGTACT TGATTTCCACAGGTTCGTTTGTAGAGATGTCAATGGCTTGCAGGGTTCCGTGTCCTTTGGGCGCCACTACCGTACCT TTGTTTTTTCTTTTGATCGGTGGTGGCTCTCTTGCTTCTTGCATGCTCAGAAGCGATGACGGGGACGCGCGCCGGGC GGAAGCGGTTGTTCCGGACCCGGAGGCATGGCTGGTAGTGGCACGTCGGCGCCGCGCACGGGCAGGTTCTGGTACTG CGCTCTGAGAAGACTTGCGTGCGCCACCACGCGTCGATTGACGTCTTGTATCTGACGTCTCTGGGTGAAAGCTACCG GCCCCGTGAGCTTGAACCTGAAAGAGAGTTCAACAGAATCAATTTCGGTATCGTTAACGGCAGCTTGTCTCAGTATT TCTTGTACGTCACCAGAGTTGTCCTGGTAGGCGATCTCCGCCATGAACTGCTCGATTTCTTCCTCCTGAAGATCTCC GCGACCCGCTCTCTCGACGGTGGCCGCGAGGTCATTGGAGATACGGCCCATGAGTTGGGAGAATGCAGTCATGCCCG CCTCGTTCCAGACGCGGCTGTAAACCACGGCCCCCTCGGAGTCTCTTGCGCGCATCACCACCTGAGCGAGGTTAAGC TCCACGTGTCTGGTGAAGACCGCATAGTTGCATAGGCGCTGAAAAAGGTAGTTGAGTGTGGTGGCAATGTGTTCGGC GACGAAGAAATACATGATCCATCGTCTCAGCGGCATTTCGCTGACATCGCCCAGAGCTTCCAAGCGCTCCATGGCCT CGTAGAAGTCCACGGCAAAATTAAAAAACTGGGAGTTTCGCGCGGACACGGTCAATTCCTCCTCGAGAAGACGGATG AGTTCGGCTATGGTGGCCCGTACTTCGCGTTCGAAGGCTCCCGGGATCTCTTCTTCCTCTTCTATCTCTTCTTCCAC TAACATCTCTTCTTCGTCTTCAGGCGGGGGCGGAGGGGGCACACGGCGACGTCGACGGCGCACGGGCAAACGGTCGA TGAATCGTTCAATGACCTCTCCGCGGCGGCGGCGCATGGTTTCAGTGACGGCGCGGCCGTTCTCGCGCGGTCGCAGA GTAAAAACACCGCCGCGCATCTCCTTAAAGTGGTGACTGGGAGGTTCTCCGTTTGGGAGGGAGAGGGCGCTGATTAT CGACGAAAGCGTCTAACCAGTCACAGTCACAAGGTAGGCTGAGTACGGCTTCTTGTGGGCGGGGGTGGTTATGTGTT CGGTCTGGGTCTTCTGTTTCTTCTTCATCTCGGGAAGGTGAGACGATGCTGCTGGTGATGAAATTAAAGTAGGCAGT TCTAAGACGGCGGATGGTGGCGAGGAGCACCAGGTCTTTGGGTCCGGCTTGCTGGATACGCAGGCGATTGGCCATTC CCCAAGCATTATCCTGACATCTAGCAAGATCTTTGTAGTAGTCTTGCATGAGCCGTTCTACGGGCACTTCTTCCTCA CCCGTTCTGCCATGCATACGTGTGAGTCCAAACCCGCGCATTGGTTGTACCAGTGCCAAGTCAGCTACGACTCTTTC GGCGAGGATGGCTTGCTGTACTTGGGTGAGGGTGGCTTGAAAGTCATCAAAATCCACAAAGCGGTGGTAAGCCCCGG TATTAATGGTGTAAGCACAGTTGGCCATGACTGACCAGTTAACTGTCTGGTGACCAGGGCGCACGAGCTCGGTGTAT TTAAGGCGCGAATAGGCGCGGGTGTCAAAGATGTAATCGTTGCAGGTGCGCACCAGATACTGGTAACCTATAAGAAA ATGCGGCGGTGGTTGGCGGTAGAGAGGCCATCGTTCTGTAGCTGGAGCGCCGGGGGCGAGGTCTTCCAACATAAGGC GGTGATAGCCGTAGATGTACCTGGACATCCAGGTGATTCCTGCGGCGGTAGTAGAAGCCCGAGGAAACTCGCGTACG CGGTTCCAAATGTTGCGTAGCGGCATGAAGTAGTTCATTGTAGGCACGGTTTGACCAGTGAGGCGCGCGCAGTCATT GATGCTCTATAGACACGGAGAAAATGAAAGCGTTCAGCGACTCGACTCCGTAGCCTGGAGGAACGTGAACGGGTTGG GTCGCGGTGTACCCCGGTTCGAGACTTGTACTCGAGCCGGCCGGAGCCGCGGCTAACGTGGTATTGGCACTCCCGTC TCGACCCAGCCTACAAAAATCCAGGATACGGAATCGAGTCGTTTTGCTGGTTGCCGAATGGCAGGGAAGTGAGTCCT ATTTTTTTTTTTTGCCGCTCAGATGCATCCCGTGCTGCGACAGATGCGTCCCCAACAACAGCCCCCCTCGCAGCAGC AGCAACCACAAAAGGCTGTCCCTGCAACTACTGCAACTGCCGCTGTGAGCGGTGCGGGACAGCCCGCCTATGATCTG GACTTGGAAGAGGGCGAAGGACTGGCACGTCTAGGTGCGCCTTCGCCCGAGCGGCATCCGCGAGTTCAACTGAAAAA AGATTCTCGCGAGGCGTATGTGCCCCAACAGAACCTATTTAGAGACAGAAGCGGCGAGGAGCCGGAGGAGATGCGAG CTTCCCGCTTTAACGCGGGTCGTGAGCTGCGTCACGGTTTGGACAGAAGACGAGTGTTGCGGGACGAGGATTTCGAA GTTGATGAAGTGACAGGGATCAGTCCTGCCAGGGCACACGTGGCTGCAGCCAACCTTGTATCGGCTTACGAACAGAC AGTAAAGGAAGAGCGTAATTTCCAAAAGTCTTTTAATAATCATGTGCGAACCCTCATTGCCCGCGAAGAAGTCACCC TTGGTTTGATGCATTTGTGGGATTTGATGGAAGCTATCATTCAGAACCCTACTAGCAAACCTCTGACCGCACAGCTG TTTCTGGTGGTGCAACACAGCAGAGACAATGAGGCTTTCAGAGAGGCGCTGCTCAACATCACCGAACCCGAGGGGAG ATGGTTGTATGATCTTATCAACATTCTACAGAGTATCATAGTGCAGGAGCGGAGCCTGGGCCTGGCCGAGAAGGTGG CTGCCATCAATTACTCGGTTTTGAGCTTGGGAAAGTATTACGCTCGCAAGATCTACAAGACTCCATACGTTCCCATA GACAAGGAGGTGAAGATAGATGGGTTCTACATGCGCATGACGCTGAAGGTGTTGACCCTGAGCGATGATCTTGGGGT GTACCGCAATGACAGAATGCATCGCGCGGTGAGCGCCAGCAGGAGGCGCGAGTTAAGCGACAGGGAACTGATGCACA GTTTGCAAAGAGCTCTAACTGGAGCTGGAACCGAGGGTGAGAATTACTTTGATATGGGAGCTGACTTGCAGTGGCAG CCTAGTCGCAGGGCTCTGAACGCCGCGACGGCAGGATGTGAGCTTCCTTACATAGAAGAGGCGGATGAAGGCGAGGA GGAAGAGGGCGAGTACTTGGAAGACTGATGGCACAACCCGTGTTTTTTGCTAGATGGAACAGCAAGCACCGGATCCC GCAATGCGGGCGGCGCTGCAGAGCCAGCCGTCCGGCATTAACTCCTCGGACGATTGGACCCAGGCCATGCAACGTAT CATGGCGTTGACGACTCGCAACCCCGAAGCCTTTAGACAGCAACCCCAGGCCAACCGTCTATCGGCCATCATGGAAG CTGTAGTGCCTTCCCGCTCTAATCCCACTCATGAGAAGGTCCTGGCCATCGTGAACGCGTTGGTGGAGAACAAAGCT ATTCGTCCAGATGAGGCCGGACTGGTATACAACGCTCTCTTAGAACGCGTGGCTCGCTACAACAGTAGCAATGTGCA AACCAATTTGGACCGTATGATAACAGATGTACGCGAAGCCGTGTCTCAGCGCGAAAGGTTCCAGCGCGATGCCAACC TGGGTTCGCTGGTGGCGTTAAATGCTTTCTTGAGTACTCAGCCTGCTAATGTGCCGCGTGGTCAACAGGATTATACT AACTTTTTAAGTGCTTTGAGACTGATGGTATCAGAAGTACCTCAGAGCGAAGTATATCAGTCCGGTCCTGATTACTT CTTTCAGACTAGCAGACAGGGCTTGCAGACGGTAAATCTGAGCCAAGCTTTTAAAAACCTTAAAGGTTTGTGGGGAG TGCATGCCCCGGTAGGAGAAAGAGCAACCGTGTCTAGCTTGTTAACTCCGAACTCCCGCCTATTATTACTGTTGGTA GCTCCTTTCACCGACAGCGGTAGCATCGACCGTAATTCCTATTTGGGTTACCTACTAAACCTGTATCGCGAAGCCAT AGGGCAAAGTCAGGTGGACGAGCAGACCTATCAAGAAATTACCCAAGTCAGTCGCGCTTTGGGACAGGAAGACACTG GCAGTTTGGAAGCCACTCTGAACTTCTTGCTTACCAATCGGTCTCAAAAGATCCCTCCTCAATATGCTCTTACTGCG GAGGAGGAGAGGATCCTTAGATATGTGCAGCAGAGCGTGGGATTGTTTCTGATGCAAGAGGGGGCAACTCCGACTGC AGCACTGGACATGACAGCGCGAAATATGGAGCCCAGCATGTATGCCAGTAACCGACCTTTCATTAACAAACTGCTGG ACTACTTGCACAGAGCTGCCGCTATGAACTCTGATTATTTCACCAATGCCATCTTAAACCCGCACTGGCTGCCCCCA CCTGGTTTCTACACGGGCGAATATGACATGCCCGACCCTAATGACGGATTTCTGTGGGACGACGTGGACAGCGATGT TTTTTCACCTCTTTCTGATCATCGCACGTGGAAAAAGGAAGGCGGCGATAGAATGCATTCTTCTGCATCGCTGTCCG GGGTCATTGGTGCTACCGCGGCTGAGCCCGAGTCTGCAAGTCCTTTTCCTAGTCTACCCTTTTCTCTACACAGTGTA CGTAGCAGCGAAGTGGGTAGAATAAGTCGCCCGAGTTTAATGGGCGAAGAGGAGTACCTAAACGATTCCTTGCTCAG ACCGGCAAGAGAAAAAAATTTCCCAAACAATGGAATAGAAAGTTTGGTGGATAAAATGAGTAGATGGAAGACTTATG CTCAGGATCACAGAGACGAGCCTGGGATCATGGGGACTACAAGTAGAGCGAGCCGTAGACGCCAGCGCCATGACAGA CAGAGGGGTCTTGTGTGGGACGATGAGGATTCGGCCGATGATAGCAGCGTATTGGACTTGGGTGGGAGAGGAAGGGG CAACCCGTTTGCTCATTTGCGCCCTCGCTTGGGTGGTATGTTGTAAAAAAAAATAAAAAAGAAAAAACTCACCAAGG CCATGGCGACGAGCGTACGTTCGTTCTTCTTTATTATCTGTGTCTAGTATAATGAGGCGAGTCGTGCTAGGCGGAGC GGTGGTGTATCCGGAGGGTCCTCCTCCTTCGTACGAGAGCGTGATGCAGCAGCAGCAGGCGACGGCGGTGATGCAAT CCCCACTGGAGGCTCCCTTTGTGCCTCCGCGATACCTGGCACCTACGGAGGGCAGAAACAGCATTCGTTACTCGGAA CTGGCACCTCAGTACGATACCACCAGGTTGTATCTGGTGGACAACAAGTCGGCGGACATTGCTTCTCTGAACTATCA GAATGACCACAGCAACTTCTTGACCACGGTGGTGCAAAACAATGACTTTACCCCTACGGAAGCCAGCACCCAGACCA TTAACTTTGATGAACGATCGCGGTGGGGCGGTCAGCTAAAAACCATCATGCATACTAACATGCCCAACGTGAACGAG TATATGTTTAGTAACAAGTTCAAAGCGCGTGTGATGGTGTCCAGAAAACCTCCTGAGGGTGTTAGAGTAGACGATAA TTATGATCATAAGCAAGATATTCTAAAATACGAGTGGTTCGAGTTTACTTTGCCAGAAGGCAACTTTTCGGTCACTA TGACTATCGACTTGATGAACAATGCCATCATAGACAATTACTTGAAAGTGGGCAGACAGAATGGAGTGTTGGAAAGT GACATTGGTGTTAAGTTCGACACTAGGAACTTCAAGTTGGGATGGGATCCAGAAACTAAGTTGATCATGCCTGGGGT TTACACCTATGAGGCCTTCCATCCTGACATCGTATTGCTGCCTGGCTGCGGAGTGGACTTTACCGAAAGCCGTCTGA GCAACCTTCTTGGCATTAGAAAGAAACACCCATTCCAAGAGGGTTTTAAGATCTTGTATGAGGATTTAGAAGGAGGA AATATTCCAGCCCTTTTGGATGTAGATGCTTATGAGAACAGCAAGAAAGATCAAAAAGCCAAAATAGAAGCTGCTGC AGAAGCTAAAGCAAACATAGTTGCCAACGATCCGGTAAGGGTGGCTAACGCTAGTGAAATCAGGGGAGACAGTTTTG CCGCAACATCCGTTCCGACTAAAGAATCATTATTGGATGATGTGTCTCAAAACATAGAGTTAAAACTCACTATTAAG CCTGTGGAAAAAGATGGCAAAAACAGAAGTTACAATGTGTTGGAAGATAAAATCAACACGGCCTATCGCAGTTGGTA CCTTTCGTACAATTATGGCGACCCCGAAAAAGGAGTGCGTTCCTGGACATTGCTCACCACCTCAGATGTCACCTGCG GAGCGGAGCAGGTCTACTGGTCGCTTCCAGACATGATGCAGGATCCTGTCACTTTCCGCTCCACTAGACAAGTCAGT AACTACCCTGTGGTGGGTGCAGAGCTTATGCCCGTCTTTTCAAAGAGCTTCTACAACGAACAAGCTGTGTACTCCCA GCAGCTCCGCCAGTCCACCTCGCTTACGCACGTCTTCAACCGCTTTCCTGAGAACCAGATTTTAATCCGTCCGCCGG CGCCCACAATTACCACCGTCAGTGAAAACGTTCCTGCTCTCACAGATCACGGGACCCTGCCGTTGCGCAGCAGTATC CGGGGAGTCCAACGTGTGACCGTTACTGACGCCAGACGCCGCACCTGTCCCTACGTGTACAAGGCACTGGGCATAGT CGCACCGCGCGTCCTTTCAAGCCGCACTTTCTAAAAAAAAAAAAAAAATGTCCGTTCTTATCTCGCCCAGTAATAAC ACCGGTTGGGGTCTGCGCGCTCCCAGCAAGATGTACGGAGGCGCACGCAAACGTTCTACCCAACATCCCGTGCGTGT TCGCGGGCATTTTCGCGCTCCATGGGGTGCCCTCAAGGGCCGCACTCGCGTTCGAACCACCGTCGATGATGTAATCG ATCAGGTGGTTGCCGACGCCCGTAATTATACTCCTACTGCGCCTACATCTACTGTGGACGCAGTTATTGACAGTGTA GTGGCTGACGCTCGCAACTATGCTCGACGTAAGAGCCGGCGAAGGCGCATTGCCAGACGTCACCGAGCTACCACTGC CATGCGAGCAGCAAGAGCTCTGCTACGAAGAGCTAGACGCGTGGGGCGAAGAGCCATGCTTAGGGCGGCCAGACGTG CAGCTTCGGGCGCCAGCGCCGGCAGGTCCCGCAGGCAAGCAGCCGCTGTCGCAGCGGCGACTATTGCCGACATGGCC CAATCGCGAAGAGGCAATGTATACTGGGTGCGTGACGCTGCCACCGGTCAACGTGTACCCGTGCGCACCCGTCCCCC TCGCACTTAGAAGATACTGAGCAGTCTCCGATGTTGTGTCCCAGCGGCGAGGATGTCCAAGCGCAAATACAAGGAAG AAATGCTGCAGGTTATCGCACCTGAAGTCTACGGCCAACCGTTGAAGGATGAAAAAAAACCCCGCAAAATCAAGCGG GTAAAAAAGGACAAAAAAGAAGAGGAAGATGGCGATGATGGGCTGGCGGAGTTTGTGCGCGAGTTTGCCCCACGGCG ACGCGTGCAATGGCGTGGGCGCAAAGTTCGACATGTGTTGAGACCTGGAACTTCGGTGGTCTTTACACCCGGCGAGC GTTCAAGCGCTACTTTTAAGCGTTCCTATGATGAGGTGTACGGGGATGATGATATTCTTGAGCAGGCAGCTGACCGA TTAGGCGAGTTTGCTTATGGCAAGCGTAGTAGAATAAATCCCAAGGATGAAACAGTGTCCATACCCTTGGATCATGG AAATCCCACCCCTAGTCTTAAACCGGTCACTTTGCAGCAAGTGTTACCCGTAACTCCGCGAACAGGTGTTAAACGCG AAGGTGAAGATTTGTATCCCACTATGCAACTGATGGTGCCCAAACGCCAGAAGTTGGAGGACGTTTTGGAGAAAGTA AAAGTGGATCCAGATATTCAACCTGAGGTTAAAGTGAGACCCATTAAGCAGGTAGCGCCTGGTCTGGGAGTACAAAC TGTAGACATTAAAATTCCCACTGAAAGTATGGAAGTGCAAACTGAACCCGCAAAGCCTACTGCCACCTCCACTGAAG TGCAAACGGACCCATGGATGCCCATGCCTATTACAACTGACGCCGTCGGTCCCACTCGAAGATCCCGACGAAAGTAC GGTCCAGCAAGTCTGTTGATGCCCAACTATGTCGTACACCCATCTATTATTCCTACTCCTGGTTACCGAGGCACTCG CTACTATCGCAGCCGAAACAGTACTTCCCGCCGTCGCCGCAAGACACCTGCAAATCGCAGTCGTCGCCGTAGACGCA CAAGCAAACCGATTCCCGGCGCCCTGGTGCGGCAAGTGTACCGCAATGGTAGTGCGGAACCTTTGACACTGCCGCGT GCGCGTTACCATCCTAGTATCATCACTTAATCAATGTTGCCGCTGCCTCCTTGCAGATATGGCCCTCACTTGTCGCC TTCGCGTTCCCATCACTGGTTACCGAGGAAGAAACTCGCGCCGTAGAAGAGGGATGTTGGGGCGCGGAATGCGACGC TACAGGCGACGGCGTGCTATCCGCAAGCAATTGCGGGGTGGTTTTTTGCCAGCCTTAATTCCAATTATCGCTGCTGC GATTGGCGCAATACCAGGCATAGCTTCCGTGGCGGTTCAGGCCTCGCAACGACATTGACATTGGAAAAAAAAAAAAC GTATAAATAAAAAATACAATGGACTCTGACACTCCTGGTACTGTGACTATGTTTTCTTAGAGATGGAAGACATCAAT TTTTCATCCTTGGCTCCGCGACACGGCACGAAGCCGTACATGGGCACCTGGAGCGACATCGGCACGAGCCAACTGAA CGGGGGCGCCTTCAATTGGAGCAGTATCTGGAGCGGGCTTAAAAATTTTGGCTCAACCATAAAAACATACGGGAACA AAGCTTGGAACAGCAGTACAGGACAGGCGCTTAGAAATAAACTTAAAGACCAGAACTTCCAACAAAAAGTAGTCGAT GGGATAGCTTCCGGTATCAATGGAGTGGTAGATTTGGCTAACCAGGCTGTGCAGAAAAAGATAAACAGTCGTTTGGA CCCGCCGCCAGCAACCCCAGGTGAAATGCAAGTGGAGGAAGAAATTCCTCCGCCAGAAAAACGAGGCGACAAGCGTC CGCGTCCCGATTTGGAAGAGACGCTGGTGACGCGCGTAGATGAACCGCCTTCTTATGAGGAAGCAACGAAGCTTGGA ATGCCCACCACTAGACCGATAGCCCCTATGGCCACCGGGGTGATGAAACCTTCTCAGTTGCATCGACCCGTCACCTT GGATTTGCCCCCTCCTCCTGCTGCTACTGCTGTACCCGCTTCTAAGCCTGTCGCTGCCCCGAAACCAGTCGCCGTAG CCAGGTCACGTCCCGGGGGCGCTCCTCGTCCAAATGCACACTGGCAAAATACTCTGAACAGCATCGTGGGTCTAGGC GTGCAAAGTGTAAAACGCCGTCGCTGCTTTTAATTAAATATGGAGTAGCGCTTAACTTGCCTATCTGTGTATATGTG TCATTACACGCCGTCACAGCATCAGAGGAAAAAAGGAAGAGGTCGTGCGTCGACGCTGAGTTACTTTCAAGATGGCC ACCCCATCGATGCTGCCCCAATGGGCATACATGCACATCGCCGGACAGGATGCTTCGGAGTACCTGAGTCCGGGTCT GGTGCAGTTCGCCCGCGCCACAGACACCTACTTCAATCTGGGAAATAAGTTTAGAAATCCTACCGTAGCGCCGACCC ACGATGTGACCACCGATCGTAGCCAGCGGCTCATGTTGCGCTTCGTGCCCGTTGACCGGGAGGACAATACATACTCT TACAAAGTGCGGTACACCCTGGCCGTGGGCGACAACAGAGTGCTGGATATGGCCAGCACGTTCTTTGACATTAGGGG CGTGTTGGACAGAGGTCCCAGTTTTAAACCCTATTCTGGTACGGCTTACAACTCCCTGGCTCCTAAAGGCGCTCCAA ATGCATCTCAGTGGTTGGATAAGGGAGTTACAAGCACTGGCCTAGTGGACGACGGCAATACTGATGATGGGGAAGAA GCCAAAAAAGCAACATACACTTTTGGTAATGCTCCAGTAAAAGCCGAGGCTGAAATCACAAAAGACGGATTGCCGGT GGGCTTGGAAGTTTCAACTGAAGGTCCTAAACCAATCTATGCTGATAAGCTTTATCAGCCAGAACCTCAAGTGGGAG ACGAAACTTGGACTGACCTAGACGGAAAAACCGAAGAGTATGGAGGGAGGGTTCTTAAACCTGAAACTAAAATGAAA CCCTGCTACGGATCTTTTGCTAAACCTACTAATATTAAAGGAGGTCAGGCAAAGGTAAAACCAAAAGAAGACGATGG CACTAACAACATCGAGTATGACATTGACATGAACTTCTTTGACTTAAGATCACAAAGATCAGAACTCAAACCTAAAA TTGTAATGTATGCAGAAAATGTGGACCTGGAATGTCCAGATACTCATGTTGTGTACAAACCTGGAGTTTCAGATGCT AGTTCTGAGACCAATCTTGGACAACAGTCTATGCCCAACAGACCCAACTACATTGGCTTCAGAGATAACTTCATCGG ACTTATGTACTATAACAGTACTGGCAACATGGGGGTACTGGCTGGCCAAGCGTCTCAGTTGAATGCAGTGGTTGACT TGCAGGACAGAAACACAGAACTGTCTTACCAACTCTTGCTTGACTCTCTGGGCGACAGAACCAGATACTTTAGCATG TGGAATCAGGCTGTGGACAGTTATGATCCTGATGTACGTGTTATTGAAAATCATGGTGTGGAAGATGAACTTCCCAA CTATTGTTTTCCGTTGGATGGTGTCGGTCCGCGAACAGATAGTTACAAGGAGATTAAGCCAAATGGAGACCAATCTA CTTGGACAAATGTAGACCCAACTGGCAGCAGTGAACTTGCTAAGGGAAATCCATTTGCCATGGAAATTAACCTTCAA GCCAATCTATGGCGAAGTTTCCTTTATTCCAATGTGGCTCTATATCTCCCAGACTCGTACAAATACACCCCGTCCAA TGTCACTCTTCCAGAAAACAAAAACACCTACGACTACATGAACGGGCGGGTGGTGCCGCCATCTCTAGTAGACACCT ATGTGAACATTGGTGCCAGGTGGTCTCTGGATGCCATGGACAATGTCAACCCATTCAACCACCACCGTAACGCTGGC TTGCGTTACCGATCCATGCTTCTGGGTAACGGACGTTATGTGCCTTTCCACATACAAGTGCCTCAAAAATTCTTCGC TGTTAAAAACCTGCTGCTTCTCCCAGGCTCCTACACTTATGAGTGGAACTTTAGGAAGGATGTAAACATGGTTCTAC AGAGTTCCCTCGGTAACGACCTACGGGTAGATGGCGCCAGCATCAGTTTTACGAGCATCAACCTCTATGCTACTTTT TTCCCCATGGCTCACAACACCGCTTCCACCCTTGAAGCCATGCTGCGGAATGACACCAATGATCAGTCATTCAACGA CTACCTATCTGCAGCTAACATGCTCTACCCCATTCCTGCCAATGCAACCAATATTCCCATTTCCATTCCTTCTCGCA ACTGGGCGGCTTTCAGAGGCTGGTCATTTACCAGACTGAAAACCAAAGAAACTCCCTCTTTGGGGTCTGGATTTGAC CCCTACTTCGTCTATTCTGGTTCTATTCCCTACCTGGATGGTACCTTCTACCTGAACCACACTTTTAAGAAGGTTTC CATCATGTTTGACTCTTCAGTGAGCTGGCCTGGAAATGACAGGTTACTATCTCCTAACGAATTTGAAATAAAGCGCA CTGTGGATGGCGAAGGCTACAACGTAGCCCAATGCAACATGACCAAAGACTGGTTCTTGGTACAGATGCTCGCCAAC TACAACATCGGCTATCAGGGCTTCTACATTCCAGAAGGATACAAAGATCGCATGTATTCATTTTTCAGAAACTTCCA GCCCATGAGCAGGCAGGTGGTTGATGAGGTCAATTACAAAGACTTCAAGGCCGTCGCCATACCCTACCAACACAACA ACTCTGGCTTTGTGGGTTACATGGCTCCGACCATGCGTCAAGGTCAACCCTATCCCGCTAACTATCCCTATCCACTC ATTGGAACAACTGCCGTAAATAGTGTTACGCAGAAAAAGTTCTTGTGTGACAGAACCATGTGGCGCATACCGTTCTC AAGCAACTTCATGTCTATGGGAGCCCTTACAGACTTGGGACAGAACATGCTCTATGCCAACTCAGCTCATGCTCTGG ACATGACCTTTGAGGTGGATCCCATGGATGAGCCCACCCTGCTTTATCTTCTCTTCGAAGTTTTCGACGTGGTCAGA GTGCATCAGCCACACCGCGGCATCATCGAGGCAGTCTACCTGCGTACACCGTTCTCGGCCGGTAACGCTACCACGTA AGAAGCTTCTTGCTTCTTGCAAACAGCAGCTGCAACCATGGCCTGCGGATCCCAAAACGGCTCCAGCGAGCAAGAGC TCAGAGCCATTGTCCAAGACCTGGGTTGCGGACCATATTTTTTGGGAACCTTTGATAAGCGCTTCCCGGGGTTCATG GCCCCCGATAAGCTCGCCTGTGCCATTGTAAATACGGCCGGACGTGAGACGGGGGGAGAGCACTGGTTGGCTTTCGG TTGGAACCCACGTTCTAACACCTGCTACCTTTTTGATCCTTTTGGATTCTCGGATGATCGTCTCAAACAGATTTACC AGTTTGAATATGAGGGTCTCCTGCGCCGCAGCGCTCTTGCTACCAAGGACCGGTGTATTACGCTGGAAAAATCTACC CAGACCGTGCAGGGCCCCCGTTCTGCCGCCTGCGGACTTTTCTGCTGCATGTTCCTTCATGCCTTTGTGCACTGGCC TGACCGTCCCATGGACGGAAACCCCACCATGAAATTGCTAACTGGAGTGCCAAACAACATGCTTCATTCTCCTAAAG TCCAGCCCACCCTGTGTGACAATCAAAAAGCACTCTACCATTTTCTCAATACCCATTCGCCTTATTTTCGCTCTCAT CGTACACACATCGAAAGGGCCACTGCGTTCGACCGTATGGATGTGCAATAATGATTCATGTAAACAACGTGTTCAAT AAACAGCACTTTATTTTTTACATGTATCGAGGCTCTGGATTACTTATTTATTTACAAGTCGAATGGGTTCTGACGAG AATCAGAATGACCCGCAGGCAGTGATACGTTGCGGAACTGATACTTGGGTTGCCACTTGAATTCGGGAATCACCAAC TTGGGAACCGGTATATCGGGCAGGATGTCACTCCACAGCTTTCTGGTCAGCTGCAAAGCTCCCAGCAGGTCAGGAGC CGAAATCTTGAAATCACAATTAGGACCAGTGCTCTGAGCGCGAGAGTTGCGGTACACCGGATTGCAGCACTGAAACA CCATCAGCGACGGATGTCTTACGCTTGCCAGCACGGTGGGATCTGCAATCATGCCCACATCCAGATCTTCAGCATTG GCAATGCTGAACGGGGTCATCTTGCAGGTCTGCCTACCCATGGCGGGCACCCAATTAGGCTTGTGGTTACAATCGCA GTGCAGGGGGATCAGTATCATCTTGGCCTGATCCTGTCTGATTCCTGGATACACGGCTCTCATGAAAGCATCATATT GCTTGAAAGCCTGCTGGGCTTTACTACCCTCGGTATAAAACATCCCGCAGGACCTGCTCGAAAACTGGTTAGCTGCG CAGCCGGCATCATTCACACAGCAGCGGGCGTCATTGTTGGCTATTTGCACCACACTTCTGCCCCAGCGGTTTTGGGT GATTTTGGTTCGCTCGGGATTCTCCTTCAAGGCTCGTTGTCCGTTCTCGCTGGCCACATCCATCTCGATAATCTGCT CCTTCTGAATCATAATATTGCCATGCAAGCACTTCAGCTTGCCCTCATAATCATTGCAGCCATGAGGCCACAACGCA CAGCCTGTACATTCCCAATTATGGTGGGCGATCTGAGAAAAAGAATGTATCATTCCCTGCAGAAATCTTCCCATCAT CGTGCTCAGTGTCTTGTGACTAGTGAAAGTTAACTGGATGCCTCGGTGCTCCTCGTTCACGTACTGGTGACAGATGC GCTTGTATTGTTCGTGCTGCTCAGGCATTAGTTTAAAAGAGGTTCTAAGTTCGTTATCCAGCCTGTACTTCTCCATC AGCAGACACATCACTTCCATGCCTTTCTCCCAAGCAGACACCAGGGGCAAGCTAATCGGATTCTTAACAGTGCAGGC AGCAGCTCCTTTAGCCAGAGGGTCATCTTTGGCGATCTTCTCAATGCTTCTTTTGCCATCCTTCTCAACGATGCGCA CGGGCGGGTAGCTGAAACCCACTGCTACAAGTTGCGCCTCTTCTCTTTCTTCTTCGCTGTCTTGACTGATGTCTTGC ATGGGGACATGTTTGGTCTTCCTTGGCTTCTTTTTCGGGGGTATCGGAGGAGGAGGACTGTCGCTCCGTTCCGGAGA CAGGGAGGATTGTGACGTTTCGCTCACCATTACCAACTGACTGTCGGTAGAAGAACCTGACCCCACACGGCGACAGG TGTTTCTCTTCGGGGGCAGAGGTGGAGGCGATTGCGAAGGGCTGCGGTCCGACCTGGAAGGCGGATGACTGGCAGAA CCCCTTCCGCGTTCGGGGGTGTGCTCCCTGTGGCGGTCGCTTAACTGATTTCCTTCGCGGCTGGCCATTGTGTTCTC CTAGGCAGAGAAACAACAGACATGGAAACTCAGCCATTGCTGTCAACATCGCCACGAGTGCCATCACATCTCGTCCT CAGCGACGAGGAAAAGGAGCAGAGCTTAAGCATTCCACCGCCCAGTCCTGCCACCACCTCTACCCTAGAAGATAAGG AGGTCGACGCATCTCATGACATGCAGAATAAAAAAGCGAAAGAGTCTGAGCCAGACATCGAACAAGACCCGGGCTAT GTGACACCGGTGGAACACGAGGAAGAGTTGAAACGCTTTCTAGAGAGAGAGGATGAAAACTGCCCAAAACAGCAAGC GGATAACTATCACCAAGATGCTGGAAATAGGGATCAGAACACCGACTACCTCATAGGGCTTGACGGGGAAGACGCGC TCCTTAAACATCTAGCAAGACAGTCACTCATAGTCAAGGATGCATTATTGGACAGAACTGAAGTGCCCATCAGTGTC GAAGAGCTCAGCCGCGCCTACGAGCTTAACCTATTTTCACCTCGTACTCCCCCCAAACGTCAGCCAAACGGCACCTG CGAGCCAAATCCTCGCTTAAACTTTTATCCAGCTTTTGCTGTGCCAGAAGTACTGGCTACCTATCACATCTTTTTTA AAAATCAAAAAATTCCAGTCTCCTGCCGCGCTAATCGCACCCGCGCCGATGCCCTACTCAATCTGGGACCTGGTTCA CGCTTACCTGATATAGCTTCCTTGGAAGAGGTTCCAAAGATCTTCGAGGGTCTGGGCAATAATGAGACTCGGGCCGC AAATGCTCTGCAAAAGGGAGAAAATGGCATGGATGAGCATCACAGCGTTCTGGTGGAATTGGAAGGCGATAATGCCA GACTCGCAGTACTCAAGCGAAGCGTCGAGGTCACACACTTTGCATACCCCGCTGTCAACCTGCCCCCTAAAGTCATG ACGGCCGTCATGGACCAGTTACTCATTAAGCGCGCAAGTCCCCTTTCAGAAGACATGCATGACCCAGATGCCTGTGA TGAGGGTAAACCAGTGGTCAGTGATGAGCAGCTAACCCGATGGCTGGGCACCGACTCTCCCCGGGATTTGGAAGAGC GTCGCAAGCTTATGATGGCCGTGGTGCTGGTTACCGTAGAACTAGAGTGTCTTCGGCGTTTCTTTACCGATTCAGAA ACCTTGCGCAAACTCGAAGAGAATCTGCACTACACTTTTAGACACGGCTTTGTGCGGCAGGCATGCAAGATATCTAA CGTGGAACTCACCAACCTGGTTTCCTACATGGGTATTCTGCATGAGAATCGCCTAGGACAAAGCGTGCTGCACAGCA CCCTTAAGGGGGAAGCCCGCCGTGATTACATCCGCGATTGTGTTTATCTCTACCTGTGCCACACGTGGCAAACCGGC ATGGGTGTATGGCAGCAATGTTTAGAAGAACAGAACCTGAAAGAGCTAAACAAGCTCTTACAGAAATCTCTTAAGGT TCTGTGGACAGGGTTCGACGAGCGCACCGTCGCTTCCGACCTGGCAGACCTCATCTTCCCAGAGCGTCTCAGGGTTA CTTTGCGAAACGGACTGCCTGACTTTATGAGCCAGAGCATGCTTAACAATTTTCGCTCTTTCATCCTGGAACGCTCC GGTATCCTGCCCGCCACCTGCTGCGCACTGCCCTCCGACTTTGTGCCTCTCACCTACCGCGAATGCCCCCCGCCGCT ATGGAGTCACTGCTACCTGTTCCGTCTGGCCAACTACCTCTCCTACCACTCGGATGTGATCGAGGATGTGAGCGGAG ACGGCTTGCTGGAGTGTCACTGCCGCTGCAATCTGTGCACGCCCCACCGGTCCCTAGCTTGCAACCCCCAGTTGATG AGCGAAACCCAGATAATAGGCACCTTTGAATTGCAAGGCCCCAGCAGCCAAGGCGATGGGTCTTCTCCTGGGCAAAG TTTAAAACTGACCCCGGGACTGTGGACCTCCGCCTACTTGCGCAAGTTTGCCCCGGAAGATTACCACCCCTATGAAA TCAAGTTCTATGAGGACCAATCACAGCCTCCGAAAGCCGAACTTTCGGCCTGCGTCATCACCCAGGGGGCAATTCTG GCCCAATTGCAAGCCATCCAAAAATCCCGCCAAGAATTTCTACTGAAAAAGGGTAAGGGGGTCTACCTTGACCCCCA GACCGGCGAGGAACTCAACACAAGGTTCCCTCAGGATGTCCCAACGACGAGAAAGCAAGAAGTTGAAGGTGCAGCCG CCGCCCCCAGAAGATATGGAGGAAGATTGGGACAGTCAGGCAGAGGAAGCGGAGGAGGAGGACAGTCTGGAGGACAG TCTGGAGGAAGACAGTTTGGAGGAGGAAAACGAGGAGGCAGAGGAGGTGGAAGAAGTAACCGCCGACAAACAGTTAT CCTCGGCTGCGGAGACAAGCAACAGCGCTACCATCTCCGCTCCGAGTCGAGGAACCCGGCGGCGTCCCAGCAGTAGA TGGGACGAGACCGGACGCTTCCCGAACCCAACCAGCGCTTCCAAGACCGGTAAGAAGGATCGGCAGGGATACAAGTC CTGGCGGGGGCATAAGAATGCCATCATCTCCTGCTTGCATGAGTGCGGGGGCAACATATCCTTCACGCGGCGCTACT TGCTATTCCACCATGGGGTGAACTTTCCGCGCAATGTTTTGCATTACTACCGTCACCTCCACAGCCCCTACTATAGC CAGCAAATCCCGGCAGTCTCGACAGATAAAGACAGCGGCGGCGACCTCCAACAGAAAACCAGCAGCGGCAGTTAGAA AATACACAACAAGTGCAGCAACAGGAGGATTAAAGATTACAGCCAACGAGCCAGCGCAAACCCGAGAGTTAAGAAAT CGGATCTTTCCAACCCTGTATGCCATCTTCCAGCAGAGTCGGGGCCAAGAGCAGGAACTGAAAATAAAAAACCGATC TCTGCGTTCGCTCACCAGAAGTTGTTTGTATCACAAGAGCGAAGATCAACTTCAGCGCACTCTCGAGGACGCCGAGG CTCTCTTCAACAAGTACTGCGCGCTGACTCTTAAAGAGTAGGCAGCGACCGCGCTTATTCAAAAAAGGCGGGAATTA CATCATCCTCGACATGAGTAAAGAAATTCCCACGCCTTACATGTGGAGTTATCAGCCCCAAATGGGATTGGCGGCAG GCGCCTCCCAGGACTACTCCACCCGCATGAATTGGCTCAGCGCCGGGCCTTCTATGATTTCTCGAGTTAATGATATA CGCGCCTACCGAAACCAAATACTTTTGGAACAGTCAGCTCTTACCACCACGCCCCGCCAACACCTTAATCCCAGAAA TTGGCCCGCCGCCCTAGTGTACCAGGAAAGTCCCGCTCCCACCACTGTATTACTTCCTCGAGACGCCCAGGCCGAAG TCCAAATGACTAATGCAGGTGCGCAGTTAGCGGGCGGCTCCACCCTATGTCGTCACAGGCCTCGGCATAATATAAAA CGCCTGATGATCAGAGGCCGAGGTATCCAGCTCAACGACGAGTCGGTGAGCTCTCCGCTTGGTCTACGACCAGACGG AATCTTTCAGATTGCCGGCTGCGGGAGATCTTCCTTCACCCCTCGTCAGGCTGTTCTGACTTTGGAAAGTTCGTCTT CGCAACCCCGCTCGGGCGGAATCGGGACCGTTCAATTTGTGGAGGAGTTTACTCCCTCTGTCTACTTCAACCCCTTC TCCGGATCTCCTGGGCACTACCCGGACGAGTTCATACCGAACTTCGACGCGATTAGCGAGTCAGTGGACGGCTACGA TTGATGTCTGGTGACGCGGCTGAGCTATCTCGGCTGCGACATCTAGACCACTGCCGCCGCTTTCGCTGCTTTGCCCG GGAACTCATTGAGTTCATCTACTTCGAACTCCCCAAGGATCACCCTCAAGGTCCGGCCCACGGAGTGCGGATTACTA TCGAAGGCAAAATACACTCTCGCCTGCAACGAATTTTCTCCCAGCGGCCCGTGCTGATCGAGCGAGACCAGGGAAAC ACCACGGTTTCCATCTACTGCATTTGTAATCACCCCGGATTGCATGAAAGCCTTTGCTGTCTTATGTGTACTGAGTT TAATAAAAACTGAATTAAGACTCTCCTACGGACTGCCGCTTCTTCAACCCGGATTTTACAACCAGAAGAACGAAACT TTTCCTGTCGTCCAGGACTCTGTTAACTTCACCTTTCCTACTCACAAACTAGAAGCTCAACGACTACACCGCTTTTC CAGAAGCATTTTCCCTACTAATACTACTTTCAAAACCGGAGGTGAGCTCCAAGGTCTTCCTACAGAAAACCCTTGGG TGGAAGCGGGCCTTGTAGTGCTAGGAATTCTTGCGGGTGGGCTTGTGATTATTCTTTGCTACCTATACACACCTTGC TTCACTTTCCTAGTGGTGTTGTGGTATTGGTTTAAAAAATGGGGCCCATACTAGTCTTGCTTGTTTTACTTTCGCTT TTGGAACCGGGTTCTGCCAATTACGATCCATGTCTAGACTTCGACCCAGAAAACTGCACACTTACTTTTGCACCCGA CACAAGCCGCATCTGTGGAGTTCTTATTAAGTGCGGATGGGAATGCAGGTCCGTTGAAATTACACACAATAACAAAA CCTGGAACAATACCTTATCCACCACATGGGAGCCAGGAGTTCCCGAGTGGTACACTGTCTCTGTCCGAGGTCCTGAC GGTTCCATCCGCATTAGTAACAACACTTTCATTTTTTCTGAAATGTGCGATCTGGCCATGTTCATGAGCAAACAGTA TTCTCTATGGCCTCCTAGCAAGGACAACATCGTAACGTTCTCCATTGCTTATTGCTTGTGCGCTTGCCTTCTTACTG CTTTACTGTGCGTATGCATACACCTGCTTGTAACCACTCGCATCAAAAACGCCAATAACAAAGAAAAAATGCCTTAA CCTCTTTCTGTTTACAGACATGGCTTCTCTTACATCTCTCATATTTGTCAGCATTGTCACTGCCGCTCACGGACAAA CAGTCGTCTCTATCCCTCTAGGACATAATTACACTCTCATAGGACCCCCAATCACTTCAGAGGTCATCTGGACCAAA CTGGGAAGCGTTGATTACTTTGATATAATCTGCAACAAAACAAAACCAATAATAGTAACTTGCAACATACAAAATCT TACATTGATTAATGTTAGCAAAGTTTACAGCGGTTACTATTATGGTTATGACAGATACAGTAGTCAATATAGAAATT ACTTGGTTCGTGTTACCCAGTTAAAAACCACGAAAATGCCAAATATGGCAAAGATTCGATCCGATGACAATTCTCTA GAAACTTTTACATCTCCCACCACACCCGACGAAAAAAACATCCCAGATTCAATGATTGCAATTGTTGCAGCGGTGGC AGTGGTGATGGCACTAATAATAATATGCATGCTTTTATATGCTTGTCGCTACAAAAAGTTTCATCCTAAAAAACAAG ATCTCCTACTAAGGCTTAACATTTAATTTCTTTTTATACAGCCATGGTTTCCACTACCACATTCCTTATGCTTACTA GTCTTGCAACTCTGACTTCTGCTCGCTCACACCTCACTGTAACTATAGGCTCAAACTGCACACTAAAAGGACCTCAA GGTGGTCATGTCTTTTGGTGGAGAATATATGACAATGGATGGTTTACAAAACCATGTGACCAACCTGGTAGATTTTT CTGCAACGGCAGAGACCTAACCATTATCAACGTGACAGCAAATGACAAAGGCTTCTATTATGGAACCGACTATAAAA GTAGTTTAGATTATAACATTATTGTACTGCCATCTACCACTCCAGCACCCCGCACAACTACTTTCTCTAGCAGCAGT GTCGCTAACAATACAATTTCCAATCCAACCTTTGCCGCGCTTTTAAAACGCACTGTGAATAATTCTACAACTTCACA TACAACAATTTCCACTTCAACAATCAGCATTATCGCTGCAGTGACAATTGGAATATCTATTCTTGTTTTTACCATAA CCTACTACGCCTGCTGCTATAGAAAAGACAAACATAAAGGTGATCCATTACTTAGATTTGATATTTAATTTGTTCTT TTTTTTTTTATTTACAGTATGGTGAACACCAATCATGGTACCTAGAAATTTCTTCTTCACCATACTCATTTGTGCAT TTAATGTTTGCGCTACTTTCACAGCAGTAGCCACAGCAACCCCAGACTGTATAGGAGCATTTGCTTCCTATGCACTT TTTGCTTTTGTTACTTGCATCTGCGTATGTAGCATAGTCTGCCTGGTTATTAATTTTTTCCAACTTCTAGACTGGAT CCTTGTGCGAATTGCCTACCTGCGCCACCATCCCGAATACCGCAACCAAAATATCGCGGCACTTCTTAGACTCATCT AAAACCATGCAGGCTATACTACCAATATTTTTGCTTCTATTGCTTCCCTACGCTGTCTCAACCCCAGCTGCCTATAG TACTCCACCAGAACACCTTAGAAAATGCAAATTCCAACAACCGTGGTCATTTCTTGCTTGCTATCGAGAAAAATCAG AAATTCCCCCAAATTTAATAATGATTGCTGGAATAATTAATATAATCTGTTGCACCATAATTTCATTTTTGATATAC CCCCTATTTGATTTTGGCTGGAATGCTCCCAATGCACATGATCATCCACAAGACCCAGAGGAACACATTCCCCTACA AAACATGCAACATCCAATAGCGCTAATAGATTACGAAAGTGAACCACAACCCCCACTACTCCCTGCTATTAGTTACT TCAACCTAACCGGCGGAGATGACTGAAACACTCACCACCTCCAATTCCGCCGAGGATCTGCTCGATATGGACGGCCG CGTCTCAGAACAGCGACTTGCCCAACTACGCATCCGCCAGCAGCAGGAACGCGCGGCCAAAGAGCTCAGAGATGTCA TCCAAATTCACCAATGCAAAAAAGGCATATTCTGTTTGGTAAAACAAGCCAAGATATCCTACGAGATCACCGCTACT GACCATCGCCTCTCTTACGAACTTGGCCCCCAACGACAAAAATTTACCTGCATGGTGGGAATCAACCCCATAGTTAT CACCCAGCAAAGTGGAGATACTAAGGGTTGCATTCACTGCTCCTGCGATTCCATCGAGTGCACCTACACCCTGCTGA AGACCCTATGCGGCCTAAGAGACCTGCTACCAATGAATTAAAAAATGATTAATAAAAAATCACTTACTTGAAATCAG CAATAAGGTCTCTGTTGAAATTTTCTCCCAGCAGCACCTCACTTCCCTCTTCCCAACTCTGGTATTCTAAACCCCGT TCAGCGGCATACTTTCTCCATACTTTAAAGGGGATGTCAAATTTTAGCTCCTCTCCTGTACCCACAATCTTCATGTC TTTCTTCCCAGATGACCAAGAGAGTCCGGCTCAGTGACTCCTTCAACCCTGTCTACCCCTATGAAGATGAAAGCACC TCCCAACACCCCTTTATAAACCCAGGGTTTATTTCCCCAAATGGCTTCACACAAAGCCCAGACGGAGTTCTTACTTT AAAATGTTTAACCCCACTAACAACCACAGGCGGATCTCTACAGCTAAAAGTGGGAGGGGGACTTACAGTGGATGACA CTGATGGTACCTTACAAGAAAACATACGTGCTACAGCACCCATTACTAAAAATAATCACTCTGTAGAACTATCCATT GGAAATGGATTAGAAACTCAAAACAATAAACTATGTGCCAAATTGGGAAATGGGTTAAAATTTAACAACGGTGACAT TTGTATAAAGGATAGTATTAACACCTTATGGACTGGAATAAACCCTCCACCTAACTGTCAAATTGTGGAAAACACTA ATACAAATGATGGCAAACTTACTTTAGTATTAGTAAAAAACGGAGGGCTTGTTAATGGCTACGTGTCTCTAGTTGGT GTATCAGACACTGTGAACCAAATGTTCACACAAAAGACAGCAAACATCCAATTAAGATTATATTTTGACTCTTCTGG AAATCTATTAACTGATGAATCAGACTTAAAAATTCCACTTAAAAATAAATCTTCTACAGCGACCAGTGAAACTGTAG CCAGCAGCAAAGCCTTTATGCCAAGTACTACAGCTTATCCCTTCAACACCACTACTAGGGATAGTGAAAACTACATT CATGGAATATGTTACTACATGACTAGTTATGATAGAAGTCTATTTCCCTTGAACATTTCTATAATGCTAAACAGCCG TATGATTTCTTCCAATGTTGCCTATGCCATACAATTTGAATGGAATCTAAATGCAAGTGAATCTCCAGAAAGCAACA TAGCTACGCTGACCACATCCCCCTTTTTCTTTTCTTACATTACAGAAGACGACAACTAAAATAAAGTTTAAGTGTTT TTATTTAAAATCACAAAATTCGAGTAGTTATTTTGCCTCCACCTTCCCATTTGACAGAATACACCAATCTCTCCCCA CGCACAGCTTTAAACATTTGGATACCATTAGAGATAGACATTGTTTTAGATTCCACATTCCAAACAGTTTCAGAGCG AGCCAATCTGGGGTCAGTGATAGATAAAAATCCATCGCGATAGTCTTTTAAAGCGCTTTCACAGTCCAACTGCTGCG GATGCGAATCCGGAGTCTGGATCACGGTCATCTGGAAGAAGAACGATGGGAATCATAATCCGAAAACGGTATCGGAC GATTGTGTCTCATCAAACCCACAAGCAGCCGCTGTCTGCGTCGCTCCGTGCAACTGCTGTTTATGGGATCAGGGTCC ACAGTGTCCTGAAGCATGATTTTAATAGCCCTTAACATCAACTTTCTGGTGCGATGCGCGCAGCAACGCATTCTGAT TTCACTCAAATCTTTGCAGTAGGTACAACACATTATTACAATATTGTTTAATAAACCATAATTAAAAGCGCTCCAGC CAAAACTCATATCTGATATAATCGCCCCTGCATGACCATCATACCAAAGTTTAATATAAATTAAATGACGTTCCCTC AAAAACACACTACCCACATACATGATCTCTTTTGGCATGTGCATATTAACAATCTGTCTGTACCATGGACAACGTTG GTTAATCATGCAACCCAATATAACCTTCCGGAACCACACTGCCAACACCGCTCCCCCAGCCATGCATTGAAGTGAAC CCTGCTGATTACAATGACAATGAAGAACCCAATTCTCTCGACCGTGAATCACTTGAGAATGAAAAATATCTATAGTG GCACAACATAGACATAAATGCATGCATCTTCTCATAATTTTTAACTCCTCAGGATTTAGAAACATATCCCAGGGAAT AGGAAGCTCTTGCAGAACAGTAAAGCTGGCAGAACAAGGAAGACCACGAACACAACTTACACTATGCATAGTCATAG TATCACAATCTGGCAACAGCGGGTGGTCTTCAGTCATAGAAGCTCGGGTTTCATTTTCCTCACAACGTGGTAACTGG GCTCTGGTGTAAGGGTGATGTCTGGCGCATGATGTCGAGCGTGCGCGCAACCTTGTCATAATGGAGTTGCTTCCTGA CATTCTCGTATTTTGTATAGCAAAACGCGGCCCTGGCAGAACACACTCTTCTTCGCCTTCTATCCTGCCGCTTAGCG TGTTCCGTGTGATAGTTCAAGTACAGCCACACTCTTAAGTTGGTCAAAAGAATGCTGGCTTCAGTTGTAATCAAAAC TCCATCGCATCTAATTGTTCTGAGGAAATCATCCACGGTAGCATATGCAAATCCCAACCAAGCAATGCAACTGGATT GCGTTTCAAGCAGGAGAGGAGAGGGAAGAGACGGAAGAACCATGTTAATTTTTATTCCAAACGATCTCGCAGTACTT CAAATTGTAGATCGCGCAGATGGCATCTCTCGCCCCCACTGTGTTGGTGAAAAAGCACAGCTAAATCAAAAGAAATG CGATTTTCAAGGTGCTCAACGGTGGCTTCCAACAAAGCCTCCACGCGCACATCCAAGAACAAAAGAATACCAAAAGA AGGAGCATTTTCTAACTCCTCAATCATCATATTACATTCCTGCACCATTCCCAGATAATTTTCAGCTTTCCAGCCTT GAATTATTCGTGTCAGTTCTTGTGGTAAATCCAATCCACACATTACAAACAGGTCCCGGAGGGCGCCCTCCACCACC ATTCTTAAACACACCCTCATAATGACAAAATATCTTGCTCCTGTGTCACCTGTAGCGAATTGAGAATGGCAACATCA ATTGACATGCCCTTGGCTCTAAGTTCTTCTTTAAGTTCTAGTTGTAAAAACTCTCTCATATTATCACCAAACTGCTT AGCCAGAAGCCCCCCGGGAACAAGAGCAGGGGACGCTACAGTGCAGTACAAGCGCAGACCTCCCCAATTGGCTCCAG CAAAAACAAGATTGGAATAAGCATATTGGGAACCGCCAGTAATATCATCGAAGTTGCTGGAAATATAATCAGGCAGA GTTTCTTGTAAAAATTGAATAAAAGAAAAATTTGCCAAAAAAACATTCAAAACCTCTGGGATGCAAATGCAATAGGT TACCGCGCTGCGCTCCAACATTGTTAGTTTTGAATTAGTCTGCAAAAATAAAAAAAAAAACAAGCGTCATATCATAG TAGCCTGACGAACAGGTGGATAAATCAGTCTTTCCATCACAAGACAAGCCACAGGGTCTCCAGCTCGACCCTCGTAA AACCTGTCATGGTGATTAAACAACAGCACCGAAAGTTCCTCGCGGTGACCAGCATGAATAATTCTTGATGAAGCATA CAATCCAGACATGTTAGCATCAGTTAACGAGAAAAAACAGCCAACATAGCCTTTGGGTATAATTATGCTTAATCGTA AGTATAGCAAAGCCACCCCTCGCGGATACAAAGTAAAAGGCACAGGAGAATAAAAAATATAATTATTTCTCTGCTGC TGTTCAGGCAACGTCGCCCCCGGTCCCTCTAAATACACATACAAAGCCTCATCAGCCATGGCTTACCAGACAAAGTA CAGCGGGCACGCACAAGCTCTAAAGTCACTCTCCAACCTCTCCACAATATATATACACAAGCCCTAAACTGACGTAA TGGGAGTAAAGTGTAAAAAATCCCGCCAAACCCAACACACACCCCGAAACTGCGTCACCAGGGAAAAGTACAGTTTC ACTTCCGCAATCCCAACAAGCGTCACTTCCTCTTTCTCACGGTACGTCACATCCCATTAACTTGCAACGTCATTTTC CCACGGCCGCGCCGCCCCGTTTAGCCGTTAACCCCACAGCCAATCACCACACACCCCACAATTTTTAAAATCACCTC ATTTACATATTGGCACCATTCCATCTATAAGGTATATTATTGATGATG

GenBank Accession No. AAW33501 MTKRVRLSDSFNPVYPYEDESTSQHPFINPGFISPNGFTQSPDGVLTLKCLTPLTTTGGSLQLKVGGGLTVDDTDGT LQENIRATAPITKNNHSVELSIGNGLETONNKLCAKLGNGLKENNGDICIKDSINTLWTGINPPPNCQIVENTNTND GKLTLVLVKNGGLVNGYVSLVGVSDTVNQMFTQKTANIQLRLYFDSSGNLLTDESDLKIPLKNKSSTATSETVASSK AFMPSTTAYPFNTTTRDSENYIHGICYYMTSYDRSLFPLNISIMLNSRMISSNVAYAIQFEWNLNASESPESNIATL TTSPFFFSYITEDDN

GenBank Accession No. ABC49791 MRRVVLGGAVVYPEGPPPSYESVMQQQQATAVMOSPLEAPFVPPRYLAPTEGRNSIRYSELAPQYDTTRLYLVDNKS ADIASLNYQNDHSNFLTTVVONNDFTPTEASTQTINFDERSRWGGQLKTIMHTNMPNVNEYMESNKFKARVMVSRKP PEGVRVDDNYDHKQDILKYEWFEFTLPEGNFSVTMTIDLMNNAIIDNYLKVGRONGVLESDIGVKEDTRNFKLGWDP ETKLIMPGVYTYEAFHPDIVLLPGCGVDFTESRLSNLLGIRKKHPFQEGFKILYEDLEGGNIPALLDVDAYENSKKD QKAKIEAAAEAKANIVANDPVRVANASEIRGDSFAATSVPTKESLLDDVSQNIELKLTIKPVEKDGKNRSYNVLEDK INTAYRSWYLSYNYGDPEKGVRSWTLLTTSDVTCGAEQVYWSLPDMMQDPVTFRSTRQVSNYPVVGAELMPVFSKSF YNEQAVYSQQLRQSTSLTHVENRFPENQILIRPPAPTITTVSENVPALTDHGTLPLRSSIRGVQRVTVTDARRRTCP YVYKALGIVAPRVLSSRTF

GenBank Accession No. AAW33485 MATPSMLPQWAYMHIAGQDASEYLSPGLVQFARATDTYFNLGNKERNPTVAPTHDVTTDRSQRLMLRFVPVDREDNT YSYKVRYTLAVGDNRVLDMASTFFDIRGVLDRGPSFKPYSGTAYNSLAPKGAPNASQWLDKGVTSTGLVDDGNTDDG EEAKKATYTFGNAPVKAEAEITKDGLPVGLEVSTEGPKPIYADKLYQPEPQVGDETWTDLDGKTEEYGGRVLKPETK MKPCYGSFAKPTNIKGGQAKVKPKEDDGTNNIEYDIDMNFFDLRSQRSELKPKIVMYAENVDLECPDTHVVYKPGVS DASSETNLGQQSMPNRPNYIGFRDNFIGLMYYNSTGNMGVLAGQASQLNAVVDLQDRNTELSYOLLLDSLGDRTRYF SMWNQAVDSYDPDVRVIENHGVEDELPNYCFPLDGVGPRTDSYKEIKPNGDQSTWTNVDPTGSSELAKGNPFAMEIN LQANLWRSFLYSNVALYLPDSYKYTPSNVTLPENKNTYDYMNGRVVPPSLVDTYVNIGARWSLDAMDNVNPENHHRN AGLRYRSMLLGNGRYVPFHIQVPQKFFAVKNLLLLPGSYTYEWNFRKDVNMVLQSSLGNDLRVDGASISFTSINLYA TFFPMAHNTASTLEAMLRNDTNDQSENDYLSAANMLYPIPANATNIPISIPSRNWAAFRGWSFTRLKTKETPSLGSG FDPYFVYSGSIPYLDGTFYLNHTFKKVSIMFDSSVSWPGNDRLLSPNEFEIKRTVDGEGYNVAQCNMTKDWFLVQML ANYNIGYQGFYIPEGYKDRMYSFFRNFQPMSRQVVDEVNYKDFKAVAIPYQHNNSGFVGYMAPTMRQGQPYPANYPY PLIGTTAVNSVTQKKFLCDRTMWRIPFSSNFMSMGALTDLGQNMLYANSAHALDMTFEVDPMDEPTLLYLLFEVEDV VRVHQPHRGIIEAVYLRTPFSAGNATT

GenBank Accession No. DQ900900 (SEQ ID NO: 217) CATCATCATAATATACCCCACAAAGTAAACAAAAGTTAATATGCAAATGAGCTTTTGAATTTTAACGGTTTTGGGGC GGAGCCAACGCTGATTGGACGAGAAGCGGTGATGCAAATAACGTCACGACGCACGGCTAACGGCCGGCGCGGAGGCG TGGCCTAGGCCGGAAGCAAGTCGCGGGGCTAATGACGTATAAAAAAGCGGACTTTAGACCCGGAAACGGCCGATTTT CCCGCGGCCACGCCCGGATATGAGGTAATTCTGGGCGGATGCAAGTGAAATTAGGTCATTTTGGCGCCAAAACTGAA TGAGGAAGTGAAAAGTGAAAAATACCTGTCCCGCCCAGGGCGGAATATTTACCGAGGGCCGAGAGACTTTGACCGAT TACGTGGGGTTTCGATTGCGGTGTTTTTTTCGCGAATTTCCGCGTCCGTGTGAAAGTCCGGTGTTTATGTCACAGAT CAGCTGATCCACAGGGTATTTAAACCAGTTGAGCCCGTCAAGAGGCCACTCTTGAGTGCCAGCGAGTAGAGATTTCT CTGAGCTCCGCTCCCAAAGTGTGAGAAAAATGAGACACCTGCGCCTCCTGTCTTCAACTGTGCCTATTAACATGGCC GCATTATTGCTGGAGGACTATGTGAGTACAGTATTGGAGGACGAACTACATCCATCTCCATTTGAGCTGGGACCTAC ACTTCAGGACCTTTATGATTTGGAGGTAGATGCCCATGATGACGACCCAAACGAAGAGGCTGTGAATTTAATATTTC CAGAATCTCTGATTCTTCAGGCTGACATAGCCAGCGAAGCTGTACCTACACCACTTCATACACCGACTTTGTCACCC ATACCTGAATTGGAAGAGGAGGACGAGTTAGACCTCCGATGTTATGAGGAAGGTTTTCCTCCCAGCGATTCAGAGGA CGAACAGGGTGAGCAGAGCATGGCTCTAATCTCAGAATATGCTTGTGTGGTTGTGGAAGAGCATTTTGTGTTGGACA ATCCTGAGGTGCCCGGGCAAGGCTGTAGATCCTGCCAGTACCACCGGGATAAGACCGGAGACACAAACGCCTCCTGC GCTCTGTGTTACATGAAAAAGAACTTCAGCTTTATTTACAGTAAGTGGAGTGAATGTGAGAGAGGCTGAGTGCTTAA CACATAACTGGGTGATGCTTAAACAGCTGTGCTAAGTGTGGTTTATTTTTGTTTCTAGGTCCGGTGTCAGAGGATGA GTCATCACCCTCAGAAGAAAACCACCCGTGTCCCCCTGAGCTGTCAGGCGAAACGCCCCTGCAAGTGCACAAACCCA CCCCAGTCAGACCCAGTGGCGAGAGGCGAGCAGCTGTTGAAAAAATTGAGGACTTGTTACATGACATGGGTGGGGAT GAACCTTTGGACCTGAGCTTGAAACGCCCCAGGAACTAGGCGCAGCTGTGCTTAGTCATGTGTAAATAAAGTTGTAC AATAAAAGTATATGTGACGCATGCAAGGTGTGGTTTATGACTCATGGGGGGGGCTTAGTCCTATATAAGTGGCAACA CCTGGGCACTGGGCACAGACCTTCAGGGAGTTCCTGATGGATGTGTGGACTATCCTTGCAGACTTTAGCAAGACACG CCGGCTTGTAGAGGATAGTTCAGACGGGTGCTCCGGGTTCTGGAGACACTGGTTTGGAACTCCTCTATCTCGACTGG TGTACACAGTTAAGAAGGATTATAACGAGGAATTTGAAAATCTTTTTGCTGATTGCTCTGGCCTGCTAGATTCTCTG AATCTCGGCCACCAGTCCCTTTTCCAGGAAAGGGTACTCCACAGCCTTGATTTTTCCAGCCCAGGGCGCACTACAGC CGGGGTTGCTTTTGTGGTTTTTCTGGTTGACAAATGGAGCCAGAACACCCAACTGAGCAGGGGCTACATTCTGGACT TCGCAGCCATGCACCTGTGGAGGGCATGGGTGAGGCAGCGGGGACAGAGAATCTTGAACTACTGGCTTATACAGCCA GCAGCTCCGGGTCTTCTTCGTCTACACAGACAAACATCCATGTTGGAGGAAGAAATGAGGCAGGCCATGGACGAGAA CCCGAGGAGCGGCCTGGACCCTCCGTCGGAAGAGGAGCTGGATTGAATCAGGTATCCAGCTTGTACCCAGAGCTTAG CAAGGTGCTGACATCCATGGCTAGGGGAGTGAAGAGGGAGAGGAGCGATGGGGGCAATACCGGGATGATGACCGAGC TGACGGCCAGCCTGATGAATCGCAAGCGCCCAGAGCGCATTACCTGGCACGAGCTACAGATGGAGTGCAGGGATGAG TTGGGCCTGATGCAGGATAAATATGGCCTGGAGCAGATAAAAACACATTGGTTGAACCCAGATGAGGATTGGGAGGA GGCCATTAAGAAATATGCCAAGATAGCCCTGCGCCCAGATTGCAAGTACATAGTGACCAAGACCGTGAATATTAGAC ATGCCTGCTACATTTCAGGGAACGGGGCAGAGGTGGTCATCGATACCCTGGACAAGGCCGCCTTCAGGTGTTGCATG ATGGGAATGAGAGCAGGAGTGATGAATATGAATTCCATGATCTTCATGAACATGAAGTTCAATGGAGAGAAGTTTAA TGGGGTGCTGTTCATGGCCAACAGCCACATGACCCTGCATGGCTGCAGTTTCTTTGGCTTCAACAATATGTGCGCCG AGGTCTGGGGCGCTTCCAAGATCAGGGGATGTAAGTTTTATGGCTGCTGGATGGGCGTGGTCGGAAGACCTAAGAGC GAGATGTCTGTGAAGCAGTGTGTGTTTGAGAAATGCTACCTGGGAGTCTCTACCGAGGGCAATGCTAGAGTGAGACA CTGCTCTTCCCTGGATACGGGCTGCTTCTGCCTGGTGAAGGGTACGGCCTCTCTGAAGCATAATATGGTGAAGGGCT GCACAGATGAGCGCATGTACAACATGCTAACATGCGACTCGGGGGTCTGTCATATCCTGAAGAACATCCATGTGACC TCCCACCCCAGAAAGAAGTGGCCAGTGTTTGAGAATAACCTGCTGATCAAGTGCCATATGCACCTGGGTGCCAGAAG GGGCACCTTCCAGCCGTACCAGTGCAACTTTAGCCAGACCAAGCTGCTGTTGGAAAACGATGCCTTCTCCAGGGTGA ACCTGAACGGCATCTTTGACATGGATGTCTCGGTGTACAAGATCCTGAGATACGATGAGACCAAGTCCAGGGTGCGC GCTTGCGAGTGCGGGGGCAGACACACCAGGATGCAGCCAGTGGCCCTGGATGTGACCGAGGAGCTGAGACCAGACCA CCTGGTGATGGCCTGTACCGGGACCGAGTTCAGCTCCAGTGGGGAGGACACAGATTAGAGGTAGGTTTGAGTAGTGG GCGTGGCTAATGTGAGTATAAAGGCGGGTGTCTTACGAGGGTCTTTTTGCTTTTCTGCAGACATCATGAACGGGACC GGCGGGGCCTTCGAAGGGGGGCTTTTTAGCCCTTATTTGACAACCCGCCTGCCGGGATGGGCCGGAGTTCGTCAGAA TGTGATGGGATCTACGGTGGATGGGCGTCCAGTGCTTCCAGCAAATTCCTCGACCATGACCTACGCGACCGTGGGGA GCTCGTCGCTTGACAGCACCGCCGCAGCCGCGGCAGCCGCAGCCGCCATGACAGCGACGAGACTGGCCTCGAGCTAT ATGCCCAGCAGCGGTAGCAGCCCCTCTGTGCCCAGTTCCATCATCGCCGAGGAGAAACTGCTGGCCCTGCTGGCCGA GCTGGAAGCCCTGAGCCGCCAGCTGGCCGCCCTGACCCAGCAGGTGTCCGATCTCCGCGAGCAACAGCAGCAGCAAA ATAAATGATTCAATAAACACAGATTCTGATTCAAACAGCAAAGCATCTTTATTATTTATTTTTTCGCGCGCGGTAGG CCCTGGTCCACCTCTCCCGATCATTGAGAGTGCGGTGGATTTTTTCCAGGACCCGGTAGAGGTGGGATTGGATGTTG AGGTACATGGGCATGAGCCCGTCCCGGGGGTGGAGGTAGCACCACTGCATGGCCTCGTGCTCTGGGGTCGTGTTGTA GATAATCCAGTCATAGCAGGGGCGCTGGGCGTGGTGCTGGATGATGTCCTTGAGGAGGAGACTGATGGCCACGGGGA GCCCCTTGGTGTAGGTGTTGGCAAAGCGGTTAAGCTGGGAGGGATGCATGCGGGGGGAGATGATGTGCAGTTTGGCC TGGATCTTGAGGTTGGCGATGTTGCCACCCAGATCCCGCCGGGGGTTCATATTGTGCAGGACCACCAGAACGGTGTA GCCCGTGCACTTGGGGAACTTATCATGCAACTTGGAAGGGAATGCGTGGAAGAATTTGGAGACGCCCTTGTGCCCGC CCAGGTTTTCCATGCACTCATCCATGATGATGGCAATGGGCCCGTGGGCTGCGGCTTTGGCAAAAACGTTTCTGGGG TCAGAGACATCATAATTATGCTCCTGGGTGAGATCATCATAAGACATTTTAATGAATTTGGGGCGAAGGGTGCCAGA TTGGGGGACGATCGTTCCCTCGGGCCCCGGGGCGAAGTTCCCCTCGCAGATCTGCATCTCCCAGGCTTTCATCTCGG AGGGGGGGATCATGTCCACCTGCGGGGCGATGAAAAAAACGGTTTCCGGGGCGGGGGTGATGAGCTGCGAGGAGAGC AGGTTTCTTAACAGCTGGGACTTGCCGCACCCGGTCGGGCCGTAGATGACCCCGATGACGGGTTGCAGGTGGTAGTT CAAGGAGATGCAGCTGCCGTCGTCCCGGAGGAGGGGGGCCACCTCGTTGAGCATGTCTCTCACTTGGAGGTTTTCCC GGACGAGCTCGCCGAGGAGGCGGTCCCCGCCCAGCGAGAGCAGCTCTTGCAGGGAAGCAAAGTTTTTCAGGGGCTTG AGCCCGTCGGCCATGGGCATCTTGGCAAGGGTCTGCGAGAGGAGCTCCAGGCGGTCCCATAGCTCGGTGACGTGCTC TACGGCATCTCGATCCAGCAGACTTCCTCGTTTCGGGGGTTGGGACGACTGCGACTGTAGGGCACGAGACGATGGGC GTCCAGCGCGGCCAGCGTCATGTCCTTCCAGGGTCTCAGGGTCCGAGTGAGGGTGGTCTCCGTCACGGTGAAGGGGT GGGCCCCGGGCTGGGCGCTTGCAAGGGTGCGCTTGAGACTCATCCTGCTGGTGCTGAAACGGGCACGGTCTTCGCCC TGCGCGTCGGCGAGATAGCAGTTGACCATGAGCTTGTAGTTAAGGGCCTCGGCGGCGTGGCCCTTGGCACGGAGCTT GCCTTTGGAAGAGCGCCCGCAGGCGGGACAGAGGAGGGATTGCAGGGCGTAGAGCTTGGGTGCGAGAAAGACGGACT CGGGAGCGAAGGCGTCCGCTCCGCAGTGGGCGCAGACGGTTTCGCACTCGACGAGCCAGGTGAGCTCGGGCTGCTCG GGGTCAAAAACCAGTTTTCCCCCGTTCTTTTTGATGCGCTTCTTACCTCGCGTCTCCATGAGTCTGTGTCCGCGTTC GGTGACAAACAGGCTGTCTGTGTCCCCGTAGACGGACTTGATTGGCCTGTCCTGCAGGGGCGTCCCGCGGTCCTCCT CGTAGAGAAACTCGGACCACTCTGAGACAAAGGCGCGCGTCCACGCCAAGACAAAGGAGGCCACGTGCGAGGGGTAG CGGTCGTTGTCCACCAGGGGGTCCACCTTTTCCACCGTGTGCAGACACATGTCCCCCTCCTCCGCATCCAAGAAGGT GATTGGCTTGTAGGTGTAGGCCACGTGACCGGGGGTCCCCGACGGGGGGGTATAAAAGGGGGCGGGTCTGTGCTCGT CCTCACTCTCTTCCGCGTCGCTGTCCACGAGCGCCAGCTGTTGGGGTAGGTATTCCCTCTCGAGAGCGGGCATGACC TCGGCACTCAGGTTGTCAGTTTCTAGAAACGAGGAGGATTTGATGTTGGCCTGCCCTGCCGCAATGCTTTTTAGGAG ACTTTCATCCATCTGGTCAGAAAAGACTATTTTTTTATTGTCAAGCTTGGTGGCAAAGGAGCCATAGAGGGCGTTGG AGAGAAGCTTGGCGATGGATCTCATGGTCTGATTTTTGTCACGGTCGGCGCGCTCCTTGGCCGCGATGTTGAGCTGG ACATACTCGCGCGCGACACACTTCCATTCTGGGAAGACGGTGGTGCGCTCGTCGGGCACGATCCTGACGCGCCAGCC GCGATTATGCAGGGTGACCAGGTCCACGCTGGTGGCCACCTCGCCGCGCAGGGGCTCGTTGGTCCAGCAGAGGCGTC CGCCCTTGCGCGAGCAGAACGGGGGCAGCACATCAAGCAGATGCTCGTCAGGGGGGTCCGCATCGATGGTGAAGATG CCCGGACAGAGTTCCTTGTCAAAATAATCGATTTTTGAGGATGCATCATCCAAGGCCATCTGCCACTCGCGGGCGGC CAGCGCTCGCTCGTAGGGGTTGAGGGGCGGACCCCAGGGCATGGGATGCGTGAGGGCGGAGGCGTACATGCCGCAGA TGTCGTAGACATAGATGGGCTCCGAGAGGATGCCGATGTAGGTGGGATAACAGCGCCCCCCGCGGATGCTGGCGCGC ACATAGTCATACAACTCGTGCGAGGGGGCCAAGAAAGCGGGGCCGAGATTGGTGCGCTGGGGCTGCTCGGCGCGGAA GACGATCTGGCGAAAGATGGCATGCGAGTTGGAGGAGATGGTGGGCCGTTGGAAGATGTTAAAGTGGGCGTGGGGCA AGCGGACCGAGTCGCGGATGAAGTGCGCGTAGGAGTCTTGCAGCTTGGCAACGAGCTCGGCGGTGACAAGGACGTCC ATGGCGCAGTAGTCCAGCGTTTCACGGATGATGTCATAACCCGCCTCTTCTTTCTTCTCCCACAGCGCGCGGTTGAG GGCGTACTCCTCGTCATCCTTCCAGTACTCCCGGAGCGGGAATCCTCGATCGTCCGCACGGTAAGAGCCCAGCATGT AGAAATGGTTCACGGCCTTGTAGGGACAGCAGCCCTTCTCCACGGGGAGGGCGTAAGCTTGAGCGGCCTTGCGGAGC GAGGTGTGCGTCAGGGCGAAGGTATCCCTAACCATGACTTTCAAGAACTGGTACTTGAAATCCGAGTCGTCGCAGCC GCCGTGCTCCCAGAGCTCGAAATCGGTGCGCTTCTTCGAGAGGGGGTTAGGCAGAGCGAAAGTGACGTCATTGAAGA GAATCTTGCCTGCCCGCGGCATGAAATTGCGGGTGATGCGGAAAGGGCCCGGAACGGAGGCTCGGTTGTTGATGACC TGGGCGGCGAGGACGATCTCGTCGAAGCCGTTGATGTTGTGCCCGACGATGTAGAGTTCCATGAATCGCGGGCGGCC TTTGATGTGCGGCAGCTTTTTGAGTTCCTCGTAGGTGAGGTCCTCGGGGCATTGCAGGCCGTGCTGCTCGAGCGCCC ACTCCTGGAGATGTGGGTTGGCTTGCATGAATGAAGCCCAGAGCTCGCGGGCCATGAGGGTCTGGAGCTCGTCGCGA AAGAGGCGGAACTGCTGGCCCACGGCCATCTTTTCTGGGGTGACGCAGTAGAAGGTGAGGGGGTCCCGCTCCCAGCG ATCCCAGCGTAAGCGCACGGCGAGATCGCGAGCGAGGGCGACCAGCTCGGGGTCCCCGGAGAATTTCATGACCAGCA TGAAGGGGACGAGCTGCTTGCCGAAGGACCCCATCCAGGTGTAGGTTTCTACATCGTAGGTGACAAAGAGCCGCTCC GTGCGAGGATGAGAGCCGATTGGGAAGAACTGGATTTCCTGCCACCAGTTGGTCGAGTGGCTGTTGATGTGATGAAA GTAGAAATCCCGCCGGCGAACCGAGCACTCGTGCTGATGCTTGTAAAAGCGTCCGCAGTACTCGCAGCGCTGCACGG GCTGTACCTCATCCACGAGATACACAGCGCGTCCCTTGAGGAGGAACTTCAGGAGTGGCGGCCCTGGCTGGTGGTTT TCATGTTCGCCTGCGTGGGACTCACCCTGGGGCTCCTCGAGGACGGAGAGGCTGACGAGCCCGCGCGGGAGCCAGGT CCAGATCTCGGCGCGGCGGGGGCGGAGAGCGAAAACGAGGGCGCGCAGTTGGGAGCTGTCCATGGTGTCGCGGAGAT CCAGGTCCGGGGGCAGGGTTCTGAGGTTGACCTCGTAGAGGCGGGTGAGGGCGTGCTTGAGATGCAGATGGTACTTG ATCTCCACGGGTGAGTTGGTGGTCGTGTCCACGCATTGCATGAGCCCGTAGCTGCGCGGGGCCACGACCGTGCCGCG GTGCGCTTTTAGAAGCGGTGTCGCGGACGCGCTCCCGGCGGCAGCGGCGGTTCCGGCCCCGCGGGCAGTGGCGGTAG AGGCACGTCGGCGTGGCGCTCGGGCAGGTCCCGGTGCTGCGCCCTGAGAGCGCTGGCGTGCGCGACGACGCGGCGGT TGACATCCTGGATCTGCCGCCTTTGCGTGAAGACCACGGGCCCCGTGACTTTGAACCTGAAAGACAGTTCAACAGAA TCAATCTCGGCGTCATTGACGGCGGCCTGACGCAGGATCTCTTGCACGTCGCCCGAGTTGTCCTGGTAGGCGATCTC GGACATGAACTGCTCGATTTCCTCCTCCTGGAGATCGCCGCGGCCCGCGCGCTCTACGGTGGCGGCAAGGTCATTCG AGATGCGACCCATGAGCTGCGAGAAGGCGCCCAGGCCGCTCTCGTTCCAGACGCGGCTGTAAACCACGTCCCCGTCG GCGTCGCGCGCGCGCATGACCACCTGCGCGAGGTTGAGCTCCACGTGCCGCGCGAAGACGGCATAGTTGCGCAGGCG TTGGAAGAGGTAGTTGAGGGTGGTGGCGATGTGCTCGGTGACGAAGAAGTACATAATCCAGCGGCGCAGGGGCATTT CGCTGATGTCGCCAATGGCCTCCAGCCTTTCCATGGCCTCGTAGAAATCCACGGCGAAGTTGAAAAACTGGGCGTTG CGGGCCGAGACCGTGAGCTCGTCTTCCAGGAGCCTGATGAGTTCGGCGATGGTGGCGCGCACCTCGCGCTCGAAATC CCCGGGGGCCTCCTCCTCTTCCTCTTCTTCCATGACGACCTCTTCTTCTATTTCTTCCTCTGGGGGCGGTGGTGGTG GCGGGGCCCGACGACGACGGCGACGCACCGGGAGACGGTCGACGAAGCGCTCGATCATCTCCCCGCGGCGGCGACGC ATGGTTTCGGTGACGGCGCGACCCCGTTCGCGAGGACGCAGCGTGAAGACGCCGCCGGTCATCTCCCGGTAATGGGG TGGGTCCCCGTTGGGCAGCGATAGGGCGCTGACAATGCATCTTATCAATTGCGGTGTAGGGCACGTGAGCGCGTCGA GATCGACCGGATCGGAGAATCTTTCGAGGAAAGCGTCTAGCCAATCGCAGTCGCAAGGTAAGCTCAAACACGTAGCA GCCCTGTGGACGCTGTTAGAATTGCGGTTGCTGATGATGTAATTGAAGTAGGCGTTTTTGAGGCGGCGGATGGTGGC GAGGAGGACCAGGTCCTTGGGTCCCGCTTGCTGGATGCGGAGCCGCTCGGCCATGCCCCAGGCCTGGCCCTGACACC GGCTCAGGTTCTTGTAGTAGTCATGCATGAGCCTCTCGATGTCATCACTGGCGGAGGCGGAGTCTTCCATGCGGGTG ACCCCGACGCCCCTGAACGGCTGCACGAGCGCCAGGTCGGCGACGACGCGCTCGGCGAGGATGGCCTGTTGCACGCG GGTGAGGGTGTCCTGGAAGTCGTCCATGTCGACGAAGCGGTGGTAGGCCCCTGTGTTGATGGTGTAAGTGCAGTTGG CCATAAGCGACCAGTTGACGGTCTGCAGGCCGGGTTGCACGACCTCGGAGTACCTGAGCCGCGAGAAGGCGCGCGAG TCGAAGACATAGTCGTTGCAGGTGCGCACGAGGTACTGGTATCCGACTAGAAAGTGCGGCGGCGGCTGGCGGTAGAG CGGCCAGCGCTGGGTGGCCGGCGCGCCCGGGGCCAGGTCCTCAAGCATGAGTCGGTGGTAGCCGTAGAGGTAGCGGG ACATCCAGGTGATGCCGGCGGCGGTGGTGGAGGCGCGCGGGAACTCGCGGACGCGGTTCCAGATGTTGCGCAGGGGC AGGAAATAGTCCATGGTCGGCACGGTCTGGCCGGTGAGACGCGCGCAGTCATTGATGCTCTAGAGGCAAAAACGAAA GCGGTTGAGCGGGCTCTTCCTCCGTAGCCTGGCGGAACGCAAACGGGTTAGGCCGCGTGTGTACCCCGGTTCGAGTC CCCTCGAATCAGGCTGGAGCCGCGACTAACGTGGTATTGGCACTCCCGTCTCGACCCAAGCCCGATAGCCGCCAGGA TACGGCGGAGAGCCCTTTTTGTCGGCCGAGGGGAGTCGCTAGACTTGAAAGCGGCCGAAAACCCTGCCGGGTAGTGG CTCGCGCCCGTAGTCTGGAGAAGCATCGCCAGGGTTGAGTCGCGGCAGAACCCGGTTCAAGGACGGCCGCGGCGAGC GGGACTTGGTCACCCCGCCGATTTAAAGACCCACAGCCAGCCGACTTCTCCAGTTACGGGAGCGAGCCCCCTTTTTT CTTTTTGCCAGATGCATCCCGTCCTGCGCCAAATGCGTCCCACCCCCCCGGCGACCACCGCGACCGCGGCCGTAGCA GGCGCCGGCGCTAGCCAGCCACAGCCACAGACAGAGATGGACTTGGAAGAGGGCGAAGGGCTGGCGAGACTGGGGGC GCCGTCCCCGGAGCGACATCCCCGCGTGCAGCTGCAGAAGGACGTGCGCCCGGCGTACGTGCCTGCGCAGAACCTGT TCAGGGACCGCAGCGGGGAGGAGCCCGAGGAGATGCGCGACTGCCGGTTTCGGGGGGCAGGGAGCTGCGCGAGGGC CTGGACCGCCAGCGCGTGCTGCGCGACGAGGATTTCGAGCCGAACGAGCAGACGGGGATCAGCCCCGCGCGCGCGCA CGTGGCGGCGGCCAACCTGGTGACGGCCTACGAGCAGACGGTGAAGCAGGAGCGCAACTTCCAAAAGAGTTTCAACA ACCACGTGCGCACCCTGATCGCGCGCGAGGAGGTGGCCCTGGGCCTGATGCACCTGTGGGACCTGGCGGAGGCCATC GTGCAGAACCCGGACAGCAAGCCTCTGACGGCACAGCTGTTCCTGGTGGTGCAGCACAGCAGGGACAACGAGGCGTT CAGGGAGGCACTGCTGAACATCGCCGAGCCCGAGGGTCGCTGGCTGCTGGAGCTGATTAACATCTTGCAGAGCATCG TAGTGCAGGAGCGCAGCCTGAGCCTGGCCGAGAAGGTGGCGGCGATCAACTACTCGGTGCTGAGCCTGGGCAAGTTT TACGCGCGCAAGATTTACAAGACGCCGTATGTGCCCATAGACAAGGAGGTGAAGATAGACAGCTTTTACATGCGCAT GGCGCTCAAGGTGCTGACGCTGAGCGACGACCTGGGCGTGTACCGCAACGACCGCATCCACAAGGCCGTGAGCACAA GCCGGCGGCGCGAGCTGAGCGACCGCGAGCTGATGCTGAGTCTGCGCCGGGCGCTGGTAGGAGGCGCCACCGGCGGT GAGGAGTCCTACTTTGACATGGGGGGGGACCTGCATTGGCAGCCGAGCCGACGCGCCTTGGAGGCCGCCTACGGTCC AGAGGACTTGGATGAGGAAGAGGAAGAGGAGGAGGATGCACCCGTTGCGGGGTACTGACGCCTCCGTGATGTGTTTT TAGATGCAGCAAGCCCCGGACCCCGCCATAAGGGCGGCGCTGCAAAGCCAGCCGTCCGGTCTAGCATCGGACGACTG GGAGGCCGCGATGCAACGCATCATGGCCCTGACGACCCGCAACCCCGAGTCCTTTAGACAACAGCCGCAGGCCAACA GACTCTCGGCCATTCTGGAGGCGGTGGTTCCTTCTCGGACCAACCCCACGCACGAGAAGGTGCTGGCGATCGTGAAC GCGCTGGCGGAGAACAAGGCCATCCGTCCCGACGAGGCCGGGCTAGTGTACAACGCCCTGCTGGAGCGCGTGGGCCG CTACAACAGCACAAACGTGCAGTCCAACCTGGACCGGCTGGTGACGGACGTGCGCGAGGCCGTGGCGCAGCGCGAGC GGTTCAAGAACGAGGGCCTGGGTTCGCTGGTGGCGCTGAACGCCTTCCTGGCGACGCAGCCGGCGAACGTGCCGCGC GGGCAGGATGATTATACCAACTTTATAAGCGCGCTGCGGCTGATGGTGACCGAGGTGCCCCAGAGCGAGGTGTACCA GTCGGGCCCGGACTACTTTTTCCAGACGAGCAGACAGGGCCTGCAGACGGTGAACCTGAGTCAGGCTTTCAAGAACC TGCGCGGGCTGTGGGGCGTGCAGGCGCCCGTGGGCGACCGGTCGACGGTGAGCAGCTTGCTGACGCCCAACTCGCGG CTGCTGCTGCTGCTGATCGCGCCCTTCACCGACAGTGGCAGCGTGAACCGCAACTCGTACCTGGGTCACCTGCTGAC GCTGTACCGCGAGGCCATAGGCCAGGCGCAGGTGGATGAGCAGACCTTCCAGGAGATCACTAGCGTGAGCCGCGCGC TGGGTCAGAACGACACCGACAGTCTGAGGGCCACCCTGAACTTCTTGCTGACCAATAGACAGCAGAAGATCCCGGCG CAGTACGCGCTGTCGGCCGAGGAGGAGCGCATCCTGAGATATGTGCAGCAGAGCGTAGGGCTGTTCCTGATGCAGGA GGGGGCCACCCCCAGCGCCGCGCTGGACATGACCGCGCGCAACATGGAACCTAGCATGTACGCCGCCAACCGGCCGT TTATTAATAAGCTGATGGACTACCTGCACCGCGCGGCGTCCATGAACTCGGACTACTTTACCAATGCCATCTTGAAC CCGCACTGGCTCCCGCCGCCGGGGTTCTACACGGGCGAGTACGACATGCCTGACCCCAACGACGGGTTTTTGTGGGA CGACGTGGACAGCGCGGTGTTCTCACCGACCTTGCAAAAGCGCCAGGAGGCGGTGCGCACGCCCGCGAGCGAGGGCG CGGTGGGTCGGAGCCCCTTTCCTAGCTTAGGGAGTTTGCATAGCTTGCCGGGCTCGGTGAACAGCGGCAGGGTGAGC CGGCCGCGCTTGCTGGGCGAGGACGAGTACCTGAACGACTCGCTGCTGCAGCCGCCGCGGGTCAAGAACGCCATGGT CAATAACGGGATAGAGAGTCTGGTGGACAAACTGAACCGCTGGAAAACCTACGCTCAGGACCATAGGGAACCTGCGC CCGCGCCGCGGCGACAGCGTCACGACCGGCAGCGGGGCCTGGTGTGGGACGACGAGGACTCGGCCGACGATAGCAGC GTGTTGGACTTGGGCGGGAGCGGTGGGGCCAACCCGTTCGCGCATCTGCAGCCCAGACTGGGGCGACGGATGTTTTG AATGCAAAATAAAACTCACCAAGGCCATAGCGTGCGTTCTCTTCCTTGTTAGAGATGAGGCGCGCGGTGGTGTCTTC CTCTCCTCCTCCCTCGTACGAGAGCGTGATGGCGCAGGCGACCCTGGAGGTTCCGTTTGTGCCTCCGCGGTATATGG CTCCTACGGAGGGCAGAAACAGCATTCGTTACTCGGAGCTGGCTCCGCTGTACGACACCACTCGCGTGTATTTGGTG GACAACAAGTCGGCGGACATCGCTTCCCTGAACTACCAAAACGACCACAGCAACTTCCTGACCACGGTGGTGCAGAA CAACGATTTCACCCCTGCCGAGGCCAGCACGCAGACGATAAATTTTGACGAGCGGTCGCGGTGGGGCGGTGATCTGA AGACCATTCTGCACACCAACATGCCTAATGTGAACGAGTACATGTTCACCAGCAAGTTTAAGGCGCGGGTGATGGTG GCTAGAAAAAAGGCGGAAGGGGCTGATGCAAATGATAGGAGCAAGGATATCTTAGAGTATCAGTGGTTTGAGTTTAC CCTGCCCGAGGGCAACTTTTCCGAGACCATGACCATAGACCTAATGAACAACGCCATCTTGGAAAACTACTTGCAAG TGGGGCGGCAAAATGGCGTGCTGGAGAGTGATATCGGAGTCAAGTTTGACAGCAGAAATTTCAAGCTGGGCTGGGAC CCGGTGACCAAGCTGGTGATGCCAGGGGTCTACACCTACGAGGCCTTCCACCCGGACGTGGTGCTGCTGCCGGGCTG CGGGGTGGATTTCACCGAGAGCCGCCTGAGCAACCTCCTGGGCATTCGCAAGAAGCAACCTTTTCAAGAGGGCTTCA GAATCATGTATGAGGACCTAGTAGGGGGCAACATCCCCGCTCTCCTGAATGTCAAGGAGTATCTGAAGGATAAGGAA GAAGCTGGCAAAGCAGATGCAAATACTATTAAGGCTCAGAATGATGCCGTCCCAAGAGGAGATAACTATGCATCAGC GGCAGAAGCCAAAGCAGCAGGAAAAGAAATTGAGTTGAAGGCCATTTTGAAAGATGATTCAGACAGAAGCTACAATG TGATCGAGGGAACCACAGACACCCTGTACCGCAGTTGGTACCTGTCCTATACCTACGGGGATCCCGAGAAGGGGGTG CAGTCGTGGACGCTGCTCACCACCCCGGACGTCACCTGCGGCGCGGAGCAAGTCTACTGGTCGCTGCCGGACCTCAT GCAAGACCCCGTCACCTTCCGCTCTACCCAGCAAGTCAGCAACTACCCCGTGGTCGGCGCCGAGCTCATGCCCTTCC GCGCCAAGAGCTTTTACAACGACCTCGCCGTCTACTCCCAGCTCATCCGCAGCTACACCTCCCTCACCCACGTCTTC AACCGCTTCCCCGACAACCAGATCCTTTGCCGCCCGCCCGCGCCCACCATCACCACCGTCAGTGAAAACGTGCCTGC TCTCACAGATCACGGGACGCTACCGCTGCGCAGCAGTATCCGCGGAGTCCAGCGAGTGACCGTCACTGACGCCCGTC GCCGCACCTGTCCCTACGTCTACAAGGCCCTGGGCATAGTCGCGCCGCGCGTGCTTTCCAGTCGCACCTTCTAAAAA ATGTCTATTCTCATCTCGCCCAGCAATAACACCGGCTGGGGTCTTACTAGGCCCAGCACCATGTACGGAGGAGCCAA GAAACGCTCCCAGCAGCACCCCGTCCGCGTCCGCGGTCACTTCCGCGCTCCCTGGGGCGCTTACAAGCGGGGGCGGA CCTCTGCTCCTGCCGCCGTGCGCACCACCGTCGACGACGTCATCGACTCGGTGGTCGCCGATGCGCGCAACTACACC CCCGCCCCCTCGACCGTGGACGCGGTCATCGACAGCGTGGTGGCAGACGCGCGTGACTATGCCAGACGCAAGAGCCG GCGGCGACGGATCGCCAGGCGCCACCGGAGCACGCCCGCCATGCGCGCCGCCCGAGCTCTGCTGCGCCGCGCCAGAC GCACGGGCCGCCGGGCCATGATGCGAGCCGCGCGCCGCGCTGCCACTGCACCCACCCCCGCAGGCAGGACTCGCAGA CGAGCGGCCGCTGCCGCCGCCGCGGCCATCTCTAGCATGACCAGACCCAGGCGCGGAAACGTGTACTGGGTGCGCGA CTCCGTCACGGGCGTGCGCGTGCCCGTGCGCACCCGTCCTCCTCGTCCCTGATCTAATGCTTGTGTCCTCCCCCGCA AGCGACGATGTCAAAGCGCAAAATCAAGGAGGAGATGCTCCAGGTCGTCGCCCCGGAGATTTACGGACCACCCCAGG CGGACCAGAAACCCCGCAAAATCAAGCGGGTTAAAAAAAAGGATGAGGTGGACGAGGGGGCAGTAGAGTTTGTGCGC GAGTTCGCTCCGCGGCGGCGCGTAAATTGGAAGGGGCGCAGGGTGCAGCGTGTGTTGCGGCCCGGCACGGCGGTGGT GTTCACGCCCGGCGAGCGGTCCTCGGTCAGGAGCAAGCGTAGCTATGACGAGGTGTACGGCGACGACGACATCCTGG ACCAGGCGGCGGAGCGGGCGGGCGAGTTCGCCTACGGGAAGCGGTCGCGCGAAGAGGAGCTGATCTCGCTGCCGCTG GACGAAAGCAACCCCACGCCGAGCCTAAAGCCCGTGACCCTGCAGCAGGTGCTGCCCCAGGCAGTGCTGCTGCCGAG CCGCGGGGTCAAGCGCGAGGGCGAGAGCATGTACCCGACCATGCAGATCATGGTGCCCAAGCGCCGGCGCGTGGAGG ACGTGCTGGACACCGTAAAAATGGATGTGGAGCCCGAGGTCAAGGTGCGCCCCATCAAGCAGGTGGCGCCGGGCCTG GGCGTGCAAACCGTGGACATTCAGATCCCCACCGACATGGATGTCGACAAAAAACCCTCGACCAGCATCGAGGTGCA AACCGACCCCTGGCTCCCAGCCTCCACCGCTACCGCGTCCACTTCTACCGCCGCCACGGCTACCGAGCCTCCCAGGA GGCGAAGATGGGGCGCCGCCAGCCGGCTGATGCCCAACTACGTGTTGCATCCTTCCATCATCCCGACGCCAGGCTAC CGCGGCACCCGGTACTACGCCAGCCGCAGGCGCCCAGCCAGCAAACGCCGCCGCCGCACCGCCACCCGCCGCCGTCT GGCCCCCGCCCGCGTGCGCCGCGTGACCACGCGGCGGGGCCGCTCGCTCGTTCTGCCCACCGTGCGCTACCACCCCA GCATCCTTTAATCCGTGTGCTGTGATACTGTTGCAGAGAGATGGCTCTCACTTGCCGCCTGCGCATCCCCGTCCCGA ATTACCGAGGAAGATCCCGCCGCAGGAGAGGCATGGCAGGCAGTGGCCTGAACCGCCGCCGGCGGCGGGCCATGCGC AGGCGCCTGAGTGGCGGCTTTCTGCCCGCGCTCATCCCCATAATCGCCGCGGCCATCGGCACGATCCCGGGCATAGC TTCCGTTGCGCTGCAGGCGTCGCAGCGCCGTTGATGTGCGAATAAAGCCTCTTTAGACTCTGACACACCTGGTCCTG TATATTTTTAGAATGGAAGACATCAATTTTGCGTCCCTGGCTCCGCGGCACGGCACGCGGCCGTTCATGGGCACCTG GAACGAGATCGGCACCAGCCAGCTGAACGGGGGCGCCTTCAATTGGAGCAGTGTCTGGAGCGGGCTTAAAAATTTCG GCTCGACGCTCCGGACCTATGGGAACAAGGCCTGGAATAGTAGCACTGGGCAGTTGTTAAGGGAAAAGCTCAAAGAC CAGAACTTCCAGCAAAAGGTGGTGGACGGGCTGGCCTCGGGCATTAACGGGGTGGTGGACATCGCGAACCAGGCCGT GCAGCGCGAGATAAACAGCCGCCTGGACCCGCGGCCGCCCACGGTGGTGGAGATGGAAGATGCAACTCTTCCGCCGC CCAAGGGCGAGAAGCGACCGCGGCCCGACGCGGAGGAGACAATCCTGCAAGTGGACGAGCCGCCCTCGTACGAGGAG GCCGTCAAGGCCGGCATGCCCACCACGCGCATCATCGCGCCGCTGGCCACGGGTGTAATGAAACCCGCTACCCTTGA CCTGCCTCCACCACCCACGCCCGCTCCACCAAAAGCAGCTCCGGTTGTGCAGCCCCCTCCGGTGGCGACCGCCGTGC GCCGCGTCCCCGCCCGCCGCCAGGCCCAGAACTGGCAGAGCACGCTGCACAGTATCGTGGGCCTGGGAGTGAAAAGT CTGAAGCGCCGCCGATGCTATTGAGAGAGAGTAAAGAGGACACTAAAGGGAGAGCTTAACTTGTATGTGCCTTACCG CCAGAGAACGCGCGAAGATGGCCACCCCCTCGATGATGCCGCAGTGGGCGTACATGCACATCGCCGGGCAGGACGCC TCGGAGTACCTGAGCCCGGGTCTGGTGCAGTTTGCCCGCGCCACCGACACGTACTTCAGCCTGGGCAACAAGTTTAG GAACCCCACGGTGGCTCCCACCCACGATGTGACCACGGACCGGTCCCAGCGTCTGACGCTGCGCTTTGTGCCCGTGG ATCGCGAGGACACCACGTACTCGTACAAGGCGCGCTTCACTCTGGCCGTGGGCGACAACCGGGTGCTAGACATGGCC AGCACTTACTTTGACATCCGCGGCGTCCTGGACCGCGGCCCAAGCTTCAAACCCTACTCGGGCACGGCTTACAACAG CCTGGCCCCCAAGGGCGCCCCCAATCCCAGTCAGTGGACTACCAAAGAAAAGCAAAACGGAGGAACTGGAGCAGAAA AAGATGTTACAAAGACATTTGGACTTGCCGCCATGGGAGGCAGTAATATTTCTAAAGACGGTTTGCAGATTGGAACT GACAAAACAGCAAATGCTGAAAAACCAATCTATGCAGACAAAACTTTCCAGCCAGAACCTCAAGTTGGAGAAGAAAA CTGGCAGGATAATGATGAATATTATGGCGGCAGGGCTCTTAAAAAAGATACCAAAATGAAGCCATGCTATGGTTCAT TTGCTAAACCCACAAACAAGGAAGGTGGGCAGGCTAAATTGAAAGAAACACCCAATGGTACCGATCCTCAATACGAT GTGGACATGGCTTTCTTTGACTCAAGCACTATAAATATACCAGATGTGGTGTTGTACACTGAAAATGTAGATTTGGA AACTCCAGATACACATGTGGTGTACAAACCAGGCAAAGAGGATGACAGTTCTGAAGCTAATTTAACTCAGCAGTCCA TGCCTAACAGACCAAACTACATTGGCTTCAGAGACAACTTTGTGGGGCTATTGTACTACAACAGCACTGGCAACATG GGTGTGCTGGCTGGTCAGGCTTCTCAGTTGAATGCCGTGGTCGACTTGCAAGACAGAAACACCGAACTGTCTTACCA GCTCTTGCTAGATTCTCTTGGTGACAGAACCAGATATTTTAGTATGTGGAACTCTGCGGTGGACAGCTATGATCCCG ATGTCAGGATCATTGAGAACCACGGTGTGGAAGATGAACTTCCTAACTATTGCTTCCCCTTGGACGGTGTTCAAACT AATTCAGCCTATCAAGGTGTTAAACTAAAGCCTGATCAAACAGGAGGCGGAGTTAATGGAGATTGGGTAAAGGATGA TGACATTTCAGCCCATAATCAAATTGGAAAGGGCAACATCTTTGCCATGGAGATCAACCTCCAGGCCAACCTGTGGA AGAGTTTTCTGTACTCGAACGTGGCCCTGTACCTGCCCGACTCCTACAAGTACACGCCGGCCAACGTCACGCTGCCC GCCAACACCAACACCTATGAGTACATGAACGGCCGCGTGGTAGCCCCCTCGCTGGTGGACGCCTACATTAACATCGG CGCCCGCTGGTCGCTGGACCCCATGGACAACGTCAACCCCTTTAACCACCACCGCAATGCGGGCCTGCGCTACCGCT CCATGCTTTTGGGCAATGGCCGCTACGTGCCCTTCCACATCCAAGTGCCCCAAAAGTTCTTTGCCATCAAGAACCTG CTCCTGCTCCCCGGCTCCTACACCTACGAGTGGAACTTCCGCAAGGATGTCAACATGATCCTGCAGAGTTCCCTCGG AAACGACCTGCGCGTCGACGGCGCCTCCGTCCGCTTCGACAGCGTCAACCTCTACGCCACCTTCTTCCCCATGGCGC ACAACACCGCCTCCACCCTGGAAGCCATGCTGCGCAACGACACCAACGACCAGTCCTTCAACGACTACCTCTCGGCC GCCAACATGCTCTACCCCATCCCGGCCAAGGCCACCAACGTGCCCATTTCCATCCCCTCGCGCAACTGGGCCGCCTT CCGCGGCTGGAGTTTCACCCGGCTCAAGACCAAGGAAACTCCCTCCCTTGGCTCGGGTTTTGACCCCTACTTTGTCT ACTCGGGCTCCATCCCCTACCTCGACGGGACCTTCTACCTCAACCACACCTTCAAGAAGGTTTCCATCATGTTCGAC TCCTCGGTCAGCTGGCCCGGCAACGACCGGCTGCTTACGCCGAACGAGTTCGAGATCAAGCGCAGCGTCGACGGGGA GGGCTACAACGTGGCCCAATGCAACATGACCAAGGACTGGTTCCTCGTCCAGATGCTCTCCCACTACAACATCGGCT ACCAGGGCTTCCATGTGCCCGAGGGCTACAAGGACCGCATGTACTCCTTCTTCCGCAACTTCCAGCCCATGAGCAGG CAGGTGGTCGATGAGATCAACTACAAGGACTACAAGGCAGTCACCCTGCCCTTCCAGCACAACAACTCTGGCTTCAC CGGCTACCTGGCACCCACCATGCGTCAGGGGCAGCCCTACCCCGCCAACTTCCCCTACCCGCTCATCGGCTCCACCG CAGTGCCATCCGTCACCCAGAAAAAGTTCCTCTGCGACAGGGTCATGTGGCGCATCCCCTTCTCCAGCAACTTCATG TCCATGGGCGCCCTCACCGACCTGGGTCAGAACATGCTCTACGCCAACTCGGCCCACGCGCTCGACATGACCTTCGA GGTGGACCCCATGGATGAGCCCACCCTCCTCTATCTTCTCTTCGAAGTTTTCGACGTGGTCAGAGTGCACCAGCCGC ACCGCGGCGTCATCGAGGCCGTCTACCTGCGCACGCCCTTCTCCGCCGGCAACGCCACCACCTAAGCATGAGCGGCT CCAGCGAACGAGAGCTCGCGGCCATCGTGCGCGACCTGGGCTGCGGGCCCTACTTTTTGGGCACCCACGACAAGCGC TTCCCGGGCTTCCTCGCCGGCGACAAGCTGGCCTGCGCCATCGTCAACACGGCCGGCCGCGAGACCGGGGGCGTGCA CTGGCTCGCCTTTGGCTGGAACCCGCGCTCGCGCACCTGCTACATGTTCGACCCCTTCGGGTTCTCGGACCGCCGGC TCAAGCAGATTTACAGCTTCGAGTACGAGGCCATGCTGCGCCGAAGCGCCCTGGCCTCCTCGCCCGATCGCTGTCTT AGCCTCGAACAGTCCACCCAGACCGTGCAGGGGCCCGACTCCGCCGCCTGCGGACTCTTCTGTTGCATGTTCTTGCA TGCCTTCGTGCACTGGCCCGACCGACCCATGGACGGGAACCCCACCATGAACTTGCTGACGGGGGTGCCCAACGGCA TGCTACAATCGCCACAGGTGCTGCCCACCCTCAGGCGCAACCAGGAGGAGCTCTACCGCTTCCTCGCGCGCCACTCC CCCTACTTTCGCTCCCACCGCGCCGCCATCGAACACGCCACCGCTTTTGATAAAATGAAACAACTGCGTGTATGACT CAAATAAACAGCACTTTTATTTTACACATGCGCTGGAGTATATGCAAGTTATTTAAAAGTCGAAGGGGTTCTCGCGC TCGTCGTTGTGCGCCGCGCTGGGGAGGGCCACGTTGCGGTACTGGAACTTGGGCTGCCACTTGAACTCGGGGATCAC CAGTTTGGGCACTGGAGTCTCGGGGAAGGTCTCGCTCCACATGCGCCGGCTCATTTGCAGGGCGCCCAGCATGTCAG GGCCGGAGATCTTGAAATCGCAGTTGGGACCGGTGCTCTGCGCGCGCGAGTTGCGGTACACGGGGTTGCAGCACTGG AACACCATCAGACTGGGGTACTTCACACTGGCAAGCACGCTCTTGTCGCTAATCTGATCCTTGTCCAGGTCCTCGGC GTTGCTCAGGCCGAACGGGGTCATCTTGCACAGCTGGCGGCCCAGGAAGGGCACGCTCTGAGGCTTGTGGTTACACT CGCAGTGCACGGGCATCAGCATCATCCCCGCGCCGCGCTGCATATTCGGGTAGAGGGCCTTGACGAAGGCCGCGATC TGCTTGAAAGCTTGCTGGGCCTTGGCCCCCTCGCTAAAAAACAGGCCGCAGCTCTTCCCGCTGAACTGGTTATTCCC GCACCCGGCATCATGCACGCAGCAGCGCGCGTCATGGCTGGTCAGTTGCACCACGCTCCGTCCCCAGCGGTTCTGGG TCACCTTAGCCTTGCTGGGCTGCTCCTTCAGCGCGCGCTGTCCGTTCTCGCTGGTCACATCCATCTCCACCACGTGG TCCTTGTGAATCATCACCGTTCCATGCAGACACTTGAGCTGACCTTCCACCTCGGTGCAGCCGTGATCCCACAGGAC GCAGCCGGTGCACTCCCAATTCTTGTGCGCGATCCCGCTGTGGCTGAAAATGTAACCTTGCAACAGGCGACCCATAA TGGTGCTAAATGCTTTCTGGGTGGTGAATGTCAGTTGCATCCCGCGGGCCTCCTCGTTCATCCAGGTCTGGCACATC TTCTGGAAGATCTCGGTCTGCTCCGGCATGAGCTTGTAAGCATCGCGCAAGCCGCTGTCGACGCGGTAGCGTTCCAT CAGCACGTTCATGGTATCCATGCCCTTCTCCCATGACGAGACCAGAGGCAGACTCAGGGGGTTGCGCACGTTCAGGA CACCAGGGGTCGCGGGCTCGACGATGCGTTTTCCGTCCTTGCCTTCCTTCAACAGAACCGGAGGCTGGCTGAATCCC ACTCCCACGATCACGGCGTCTTCCTGGGGCATCTCTTCGTCGGGGTCTACCTTGGTCACATGCTTGGTCTTTCTGGC TTGCTTCTTTTTTGGAGGGCTGTCCACGGGGACCACGTCCTCCTCGGAAGACCCGGAGCCCACCCGCTGATACTTTC GGCGCTTGGTGGGCAGAGGAGGTGGCGGCGGCGAGGGGCTCCTCTCCTGCTCCGGCGGATAGCGCGCCGACCCGTGG CCCCGGGGCGGAGTGGCCTCTCGCTCCATGAACCGGCGCACGTCCTGACTGCCGCCGGCCATTGTTTCCTAGGGGAA GATGGAGGAGCAGCCGCGTAAGCAGGAGCAGGAGGAGGACTTAACCACCCACGAGCAACCCAAAATCGAGCAGGACC TGGGCTTCGAAGAGCCGGCTCGTCTAAAACCCCCACAGGATGAACAGGAGCACGAGCAAGACGCAGGCCAGGAGGAG ACCGACGCTGGGCTCGAGCATGGCTACCTGGGAGGAGAGGAGGATGTGCTGCTAAAACACCTGCAGCGCCAGTCCCT CATCCTCCGGGACGCCCTGGCCGACCGGAGCGAAACCCCCCTCAGCGTCGAGGAGCTGTGTCGGGCCTACGAGCTCA ACCTCTTCTCGCCGCGCGTGCCCCCCAAACGCCAGCCCAACGGCACCTGCGAGCCCAACCCGCGTCTCAACTTCTAT CCCGTCTTTGCGGTCCCCGAGGCCCTTGCCACCTATCACATCTTTTTCAAGAACCAAAAGATCCCCATCTCCTGTCG CGCCAATCGCACTCGCGCCGACGCGCTCCTCGCTCTGGGGCCCGGCGCGCGCATACCTGATATCGCTTCCCTGGAAG AGGTGCCCAAGATCTTCGAAGGGCTCGGTCGGGACGAGACGCGCGCGGCAAACGCTCTGAAAGAAACAGCAGAGGAA GAGGGTTACACTAGCGCCCTGGTAGAGTTGGAAGGCGACAACGCCAGGCTGGCCGTGCTTAAGCGCAGCGTCGAGCT CACCCATTTCGCCTACCCCGCCGTCAACCTCCCGCCCAAGGTCATGCGTCGCATCATGGATCAGCTCATCATGCCCC ACATCGAGGCCCTTGATGAAAGTCAGGAACAGCGCCCCGAGAACGCCCAGCCCGTGGTCAGCGACGAGATGCTCGCG CGCTGGCTCGGGACCCGCGACCCCCAGGCCCTGGAGCAGCGGCGCAAGCTCATGCTGGCCGTGGTCCTGGTCACCCT TGAGCTCGAATGCATGCGCCGCTTTTTTACCGACCCCGAGACCCTGCGCAAGGTCGAGGAGACCCTGCACTACACTT TCAGACACGGTTTCGTCAGGCAGGCCTGCAAGATCTCCAACGTGGAGCTGACCAACCTGGTCTCCTGCCTGGGGATC CTACACGAGAACCGCTTGGGACAGACCGTGCTCCACTCTACCCTGAAGGGCGAGGCGCGGCGGGACTACATCCGCGA CTGCGTCTTTCTCTTTCTCTGCCACACATGGCAAGCGGCCATGGGCGTGTGGCAGCAGTGTCTCGAGGACGAGAACC TGAAGGAGCTGGACAAGCTTCTTGCTAGAAACCTTAAAAAGCTGTGGACGGGCTTCGACGAGCGCACCGTCGCCTCG GACCTGGCCGAGATCGTCTTCCCCGAGCGCCTGAGGCAGACGCTGAAAGGAGGGCTGCCCGACTTCATGAGCCAGAG CATGTTGCAAAACTACCGCACTTTCATTCTCGAGCGATCTGGGATGCTGCCCGCCACCTGCAACGCCTTCCCCTCCG ACTTTGTCCCGCTGAGCTACCGCGAGTGTCCCCCGCCGCTGTGGAGCCACTGCTACCTCTTGCAGCTGGCCAACTAC ATTGCCCACCACTCGGATGTGATCGAGGACGTGAGCGGCGAGGGGCTGCTCGAGTGCCACTGTCGCTGCAACCTATG CTCCCCGCACCGCTCCCTGGTCTGCAACCCCCAGCTACTGAGCGAGACCCAGGTCATCGGTACCTTTGAGCTGCAAG GTCCGCAGGAGTCCACCGCTCCGCTGAAACTCACGCCGGGGTTGTGGACTTCCGCGTACCTGCGCAAATTTGTACCC GAGGACTACTACGCCCATGAGATAAAGTTCTTCGAGGACCAATCGCGTCCGCAGCACGCGGATCTCACGGCCTGCGT CATCACCCAGGGCGCGATCCTCGCCCAATTGCACGCCATCCAAAAATCCCGCCAAGAGTTTCTTCTGAAAAAGGGTA GAGGGGTCTACCTGGACCCCCAGACGGGCGAGGTGCTCAACCCGGGTCTCCCCCAGCATGCCGAGGAAGAAGCAGGA GCCGCTAGTGGAGGAGATGGAAGAAGAATGGGACAGCCAGGCAGAGGAGGACGAATGGGAGGAGGAGACAGAGGAGG AAGACTTGGAAGAGGTGGAAGAGGAGCAGGCAACAGAGCAGCCCGTCGCCGCACCATCCGCGCCGGCAGCCCCTCCG GTCACGGATACAACCTCCGCAGCTCCGGCCAAGCCTCCTCGTAGATGGGATCGAGTGAAGGGTGACGGTAAGCACGA GCGACAGGGCTACCGATCATGGAGGGCCCACAAAGCCGCGATCATCGCCTGCTTGCAAGACTGCGGGGGGAACATCG CTTTCGCCCGCCGCTACCTGCTCTTCCACCGCGGGGTGAACATCCCCCGCAACGTGTTGCATTACTACCGTCACCTT CACAGCTAAGAAAAAGCAAGTCAAAGGAGTCGCCGGAGGAGGAGGCCTGAGGATCGCGGCGAACGAGCCCTTGACCA CCAGGGAGCTGAGGAACCGGATCTTCCCCACTCTTTATGCCATTTTTCAGCAAAGTCGAGGTCAGCAGCAAGAGCTC AAAGTAAAAAACCGGTCTCTGCGCTCGCTCACCCGCAGTTGCTTGTACCACAAAAACGAAGATCAGCTGCAGCGCAC TCTCGAAGACGCCGAGGCTCTGTTCCACAAGTACTGCGCGCTGACTCTTAAAGACTAAGGCGCGCCCACCCGGAAAA AAGGCGGGAATTACCTCATCGCCACCATGAGCAAGGAGATTCCCACCCCTTACATGTGGAGCTATCAGCCCCAGATG GGCCTGGCCGCGGGCGCCTCCCAGGACTACTCCACCCGCATGAACTGGCTTAGTGCCGGCCCCTCGATGATCTCACG GGTCAACGGGGTCCGTAACCATCGAAACCAGATATTGTTGCAGCAGGCGGCGGTCACCTCCACGCCCAGGGCAAAGC TCAACCCGCGTAATTGGCCCTCCACCCTGGTGTATCAGGAAATCCCCGGGCCGACTACCGTACTACTTCCGCGTGAC GCACTGGCCGAAGTCCGCATGACTAACTCAGGTGTCCAGCTGGCCGGCGGCGCTTCCCGGTGCCCGCTCCGCCCACA ATCGGGTATAAAAACCCTGGTGATCCGAGGCAGAGGCACACAGCTCAACGACGAGTTGGTGAGCTCTTCAATCGGTC TGCGACCGGACGGAGTGTTCCAACTAGCCGGAGCCGGGAGATCGTCCTTCACTCCCAACCAGGCCTACCTGACCTTG CAGAGCAGCTCTTCGGAGCCTCGCTCGGGAGGCATCGGAACCCTCCAGTTCGTGGAGGAGTTTGTGCCCTCGGTCTA CTTCAACCCCTTCTCGGGCTCGCCAGGCCTCTACCCGGACGAGTTTATACCGAACTTCGACGCAGTGAGAGAAGCGG TGGACGGCTACGACTGAATGTCCTATGGTGACTCGGCTGAGCTCGCTCGGTTGAGGCATCTGGACCACTGCCGCCGC CTGCGCTGCTTTGCCCGGGAGAGCTACGGCCTCATCTACTTTGAGCTGCCCGAGGAGCACCCCAACGGCCCTGCACA CGGAGTGCGGATCACCGTAGAGGGCACCACCGAGTCTCACCTGGTCAGGTTCTTCACCCAGCAACCCTTCCTGGTCG AGCGGGACCGGGGCGCCACCACCTACACCGTCTACTGCATTTGTCCTACCCCGAAGTTGCATGAGAATTTTTGTTGT ACTCTTTGTGGTGAGTTTAATAAAAGCTAAACTCTTGCAATACTCTGGACCTTGTCGTCATCAACTCAACGAGACCG TCTACCTCACCAACCAGACTGAGGTAAAACTTACCTGCAGACCACACAAGACCTATATCATCTGGTTCTTCGAGAAC ACCTCATTTGCAGTCTCCAACACTCACTGCAACGACGGTGTTGAACTTCCCAACAACCTTTCCAGTGGACTGAGTTA CAATACACGTAGAGCTAAGCTCATCCTCTACAATCCTTTTGTAGAGGGAACCTACCAGTGCCAGAGCGGACCTTGCT TCCACAGTTTTACTTTGGTGAACGTTACCGGCAGCAGCACAGCCGCTCCAGAAACTAACCTTCCTTCTGATACTATC AAACCTTGTTTCGGAGGTGAGCTAAGGCTTCCCCCTTCTCAGGAGGGGGTTAGCCCATACGAAGTGGTCGGGTATTT GATTTTAGGGGTGGTCCTGGGTGGGTGCATAGCGGTGCTAGCTCAGCTGCCTTGCTGGGTGGAAATCAAAATCTTTA TATGCTGGGTAAGACATTGTGGGGAGGAACTATGAAGGGGCTCTTGCTGATTATCCTTTCCCTGGTGGGGGGTGTGC TGTCATGCCACGAACAGCCACGATGTAACATCACCACAGGCAATGAGAGGAACGACTGCTCTGTAGTTATCAAATGC GAGCACCATTGTCCTCTCAACATTACATTCAAAAATAAGACCATGGGAAATGTATGGGTGGGATTCTGGCAACCAGG AGATGAGCAGAACTACACGGTCACTGTCCATGGTAGCAATGGCAATCACACTTTCGGTTTCAAATTCATTTTTGAAG TCATGTGTGATATCACACTACATGTGGCTAGACTTCATGGCTTGTGGCCCCCTACCAAGGATAACATGGTGGGTTTT TCTTTGGCTTTTGTGATCATGGCCTGCTTGATGTCAGGTCTGCTGGTAGGGGCTCTAGTGTGGTTTCTGAAACGCAA GCCCAGGTATGGAAATGAAGAGAAGGAAAAATTGCTATAAATTCTTTTTCTTTTTCGCAGAACCATGAATACAGTGA TCCGTATCGTGCTGCTCTCTCTTCTTGTAGCTTTTAGTCAGGCAGGATTTCATACTATCAATGCTACATGGTGGGCT AATATAACTTTAGTGGGACCCCCAGACACACCAGTCACTTGGTATGATACTCAAGGATTGTGGTTTTGCAATGGCAG TAGAGTTAAGAATCCTCAAATCAGACATACATGTAATGATCAAAACCTTACTTTGATCCATGTGAACAAAACTTATG AAAGAACATACATGGGTTATAATAGACAAGGGACTAAAAAAGAAGACTACAAAGTTGTAGTTATACCACCTCCTCCT GCTACTGTAAAACCACAGCCAGAGCCAGAGTATGTGTTTGTTTATATGGGAGAGAACAAAACTCTAGAAGGTCCTCC GGGAACTCCAGTCACATGGTTTAATCAGGATGGAAAGAAATTTTGTGAAGGAGAAAAAGTTCTTCATCCAGAATTTA ACCACACCTGTGACAAACAAAACCTTATACTACTGTTTGTGAATTTTACACATGATGGAGCTTACCTTGGGTACAAT CATCAAGGAACCCAGAGAACACACTATGAAGTTACAGTATTAGATCTTTTTCCAGATTCTGGCCAAATGAAAATTGA AAATCATAGTGAGGAAACAGAGCAAAAAAATGATGAACATCATAACTGGCAGAAACAGGGTGGGCAAAAACAGGGTG GGCAAAAAACAAATCAAACAAAAGTTAATGACAGGAGAAAAACAGCGCAAAAAAGACCATCAAAGCTAAAGCCGGCA ACTATTGAGGCAATGCTGGTTACAGTGACTGCCGGGTCTAACTTAACTTTGGTTGGACCTAAAGCAGAAGGAAAAGT TACTTGGTTTGATGGAGATTTAAAAAGACCATGTGAGCCTAATTACAGACTAAGACACGAATGTAATAATCAAAACT TAACTCTGATTAATGTAACTAAAGATTATGAGGGAACTTACTATGGTACAAATGACAAAGATGAGGGCAAAAGGTAC AGAGTGAAAGTAAATACTACAAATTCTCAATCTGTGAAAATTCAGCCATATACCAGACAAACTACTCCTGATCAAGA GCACAAATTTGAATTACAGTTCGAAACTAATGGAAATTATGATTCAAAAATTCCCTCAACCACTGTGGCAATCGTGG TGGGTGTGATTGCGGGCTTCATAACTCTGATCATTGTCTTCATATGCTACATCTGCTGCCGCAAGCGTCCCAGGGCA TACAATCATATGGTAGACCCACTACTCAGCTTCTCTTACTAAGACTCAGTCACTTTCATTTCAGAACCATGAAGGCT TTCACAGCTTGCGTTCTGATTAGCCTAGTCACACTTAGTGTAGCTATTAAAAATCAATATCATGTTCATAATGTTAC CAGAGATGGATATATCACATTAAATGTAACAATTGATAATACTACCTGGACAAGATATCATTTAAATAAGTGGCATC AAATTTGTACGTGGTCAGACCCATCATACAAATGTCACAGCAATGGCAGCATTACCATTCATGCTTTCAATATTACT TCTGGCCAGTACAAAGCTGAAAGTTTTACTAACTGGTTTAGATATTACGGTAATCATAAACATGAAATTCATATTTT TAACATAACTGTAATTGAGCATCCTACAACAAAAGCACCCACCACTGCTAATACAGCTACATCAATTAAATCAACAA CCACACAGCCTACTACTAGGGAGACAACTCAACCTACCACCACAGTCAGTACAACTACTGAGACCACTACTCAAACT ACACAGCTAGACACAACAGTGCAGAATAGCACTGTGTTGGTTAGGTATCTGTTGAGGGAGGAAAGTACTACTGAACA GACAGAGGCTACCTCAAGTGCCTTTAGCAGCACTGCAAATTTAACTTCGCTTGCTTGGACTAATGAAACCGGAGTAT CATTGATGAATCATCAGCCTTTCTCAGGTTTGGATATTCAAATTACTTTTCTGGTTGTTTGTGGGATCTTTATTCTT GTGGTTCTTCTGTACTTTGTCTGCTGCAAAGCCAGAGAGAAATCTAGGAGGCCCATCTACAGGCCAGTAATCGGGGA ACCTCAGCCACTCCAAGTGGAAGGGGGTCTAAGGAATCTTCTTTTCTCTTTTTCAGTATGGTGATCAGCCATGATTC CTAGGTTCTTCCTATTTAACATCCTCTTCTGTCTCTTCAACATCTGCGCTGCCTTTGCAGCCGTCTCGCACGCCTCG CCCGACTGTCTCGGGCCCTTCCCAACCTACCTCCTCTTTGCCCTGCTCACCTGCACCTGCGTCTGCAGCATTGTCTG CCTGGTCATCACCTTCCTGCAGCTTATCGACTGGTGCTGTGCGCGCTACAATTATCTCCATCACAGTCCCGAATACA GGGACAAGAACGTAGCCAGAATCTTAAGGCTCATCTGACCATGCAGACTCTGCTCATGCTGCTATCCCTCCTATCCC CTGCCCTAGCCACTTATGCTGATTACTCTAAATGCAAATTCGCAGACATATGGAATTTCTTAGATTGCTATCAGGAA AAAATTGATATGCCCTCCTATTACTTGGTGATTGTGGGAATAGTCATGGTCTGCTCCTGCACTTTCTTTGCCATCAT GATTTACCCCTGTTTTGATCTCGGCTGGAACTCTGTTGAAGCATTCACATACACACTAGAAAGCAGTTCACTAGCCT CCACGCCACCACCCACACCGCCTCCTCGCAGAAATCAGTTCCCCCTGATACAGTACTTAGAAGAGCCCCCTCCCCGA CCCCCTTCCACTGTTAGCTACTTTCACATAACCGGCGGCGATGACTGACCACCACCTGGACCTCGAGATGGACGGCC AGGCCTCCGAGCAGCGCATCCTGCAACTGCGCGTCCGTCAGCAGCAGGAGCGGGCCGCCAAGGAGCTCCTTGATGCC ATCAACATCCACCAGTGCAAGAAGGGCATCTTCTGCCTGGTCAAACAGGCAAAGATCACCTACGAGCTCGTGTCCAA CGGCAAACAGCATCGCCTTACCTATGAGATGCCCCAGCAGAAGCAGAAGTTCACCTGCATGGTGGGCGTCAACCCCA TAGTCATCACCCAGCAGTCGGGCGAGACCAACGGCTGCATCCACTGCTCCTGCGAAAGCCCCGAGTGCATCTACTCC CTTCTCAAGACCCTTTGCGGACTCCGCGACCTCCTCCCCATGAACTGATGTTGATTAAAAGCCCAGAAACCAATCAG ACCCTTCCTCATTTCCCCATCCCAATACTCATAAGAATAAATCATTGGAATTAATCATTCAATAAAGATCACTTACT TGAAATCTGAAAGTATGTCTCTGGTGTAGTTGCTCAGCAACACCTCGGTACCCTCCTCCCAGCTCTGGTACTCCAGT CCCCGGCGGGCGGCGAACTTCCTCCACACCTTGAAAGGGATGTCAAATTCCTGGTCCACAATTTTCATTGTCTTCCC TCTTAGATGTCAAAGAGGCTCCGGGTGGAAGATGACTTCAACCCCGTCTACCCCTATGGCTACGCGCGGAATCAGAA TATCCCCTTCCTCACTCCCCCCTTTGTCTCCTCCGATGGATTCAAAAACTTCCCCCCTGGGGTACTGTCACTCAAAC TGGCTGATCCAATCACCATTACCAATGGGGATGTATCCCTCAAGGTGGGAGGTGGTCTCACTTTGCAAGATGGAAGC CTAACTGTAAACCCTAAGGCTCCACTGCAAGTTAATACTGATAAAAAACTTGAGCTTGCATATGATAATCCATTTGA AAGTAGTGCTAATAAACTTAGTTTAAAAGTAGGACATGGATTAAAAGTATTAGATGAAAAAAGTGCTGCGGGGTTAA AAGATTTAATTGGCAAACTTGTGGTTTTAACAGGAAAAGGAATAGGCACTGAAAATTTAGAAAATACAGATGGTAGC AGCAGAGGAATTGGTATAAATGTAAGAGCAAGAGAAGGGTTGACATTTGACAATGATGGATACTTGGTAGCATGGAA CCCAAAGTATGACACGCGCACACTTTGGACAACACCAGACACATCTCCAAACTGCACAATTGCTCAAGATAAGGACT CTAAACTCACTTTGGTACTTACAAAGTGTGGAAGTCAAATATTAGCTAATGTGTCTTTGATTGTGGTCGCAGGAAAG TACCACATCATAAATAATAAGACAAATCCAAAAATAAAAAGTTTTACTATTAAACTGCTATTTAATAAGAACGGAGT GCTTTTAGACAACTCAAATCTTGGAAAAGCTTATTGGAACTTTAGAAGTGGAAATTCCAATGTTTCGACAGCTTATG AAAAAGCAATTGGTTTTATGCCTAATTTGGTAGCGTATCCAAAACCCAGTAATTCTAAAAAATATGCAAGAGACATA GTTTATGGAACTATATATCTTGGTGGAAAACCTGATCAGCCAGCAGTCATTAAAACTACCTTTAACCAAGAAACTGG ATGTGAATACTCTATCACATTTAACTTTAGTTGGTCCAAAACCTATGAAAATGTTGAATTTGAAACCACCTCTTTTA CCTTCTCCTATATTGCCCAAGAATGAAAGACCAATAAACGTGTTTTTCATTTGAAATTTTCATGTATCTTTATTGAT TTTTACACCAGCACGAGTAGACAGTCTCCCACCACCAGCCCATTTTACAGTGTACACGGTTCTCTCAGCACGGGTAG CCTTAAATAGGGAAATATTCTCATTAGTGCGGGAATTGGACTTGGGGTCTATAATCCACACAGTTTCCTGGCGAGCC AAACGGGGGTCGGTGATTGAAATAAAGCCGTCCTCTGAAAAGTCATCCAAGCGGGCCTCACAGTCCAAGGTCACAGT CTGGTGGAACGAGAAGAACGCACAGATTCATACTCGGAAAACAGGATGGGTCTGTGCCTCTCCATCAGCGCCCTCAG CAGTCTCTGCCGCCGGGGCTCGGTGCGGCTGCTGCAAATGGGATCGGGATCACAAGTCTCTCTGACTATGATCCCAA CAGCCTTCAGCATCAGTCTCCTGGTGCGACGGGCACAGCACCGCATCCTGATCTCTGCCATGTTCTCACAGTAAGTG CAGCACATAATCACCATGTTATTCAGCAGCCCATAATTCAGGGCGCTCCAGCCAAAGCTCATGTTGGGAATGATGGA ACCCACGTGACCATCGTACCAGATGCGACAGTATATCAGGTGCCTGCCCCTCATGAACACACTGCCCATGTACATGA TCTCTTTGGGCATGTTTCTGTTTACAATCTGGCGGTACCAGGGGAAGCGCTGGTTGAACATGCACCCGTAAATGACT CTCCTGAACCACACGGCCAGCAGGGTGCCTCCCGCCCGACACTGCAGGGAGCCAGGGGATGAACAGTGGCAATGCAG GATCCAGCGCTCGTACCCGCTCACCATTTGAGCTCTTACCAAGTCCAGGGTAGCGGGGCACAGGCACACTGACATAC ATCTTTTTAAAATTTTTATTTCCTCTGTGGTGAGGATCATATCCCAGGGGACTGGAAACTCTTGGAGCAGGGTAAAG CCAGCAGCACATGGTAATCCACGGACAGAACTTACATTATGATAATCTGCATGATCACAATCGGGCAACAGGGGATG TTGTTCAGTCAGTGAAGCCCTGGTTTCCTCATCAGATCGTGGTAAACGGGCCCTGCGATATGGATGATGGCGGAGCG AGCTGGATTGAATCTCGGTTTGCATTGTAGTGGATTCTCTTGCGTACCTTGTCGTACTTCTGCCAGCAGAAATGGGC CCTTGAACAGCATATACCCCTCCTACGGCCGTCCTTTCGCTGCTGCCGCTCAGTCATCCAACTAAAGTACATCCATT CTCGAAGATTCTGGAGAAGTTCCTCTGCATCTGATAAAATAAAAAACCCGTCCATGCGAATTCCCCTCATCACATCA GCCAGGACTCTGTAGGCCATCCCCATCCAGTTAATGCTGCCTTGTCTATCATTCAGAGGGGGCGGTGGCAGGACTGG AAGAACCATTTTTATTCCAAACGGTCTCGAAGGACGATAAAGTGCAAGTCACGCAGGTGACAGCGTTCCCCTCCGCT GTGCTGGTGGAAACAGACAGCCAGGTCAAAACCCACTCTATTTTCAAGGTGCTCGACCGTGGCTTCGAGCAGTGGCT CTACGCGCACATCCAGCATAAGAATCACATTAAAGGCTGGCCCTCCATCGATTTCATCAATCATCAGGTTACATTCC TGCACCATCCCCAGGTAATTCTCATTTTTCCAGCCTTGGATTATCTCTACAAATTGTTGGTGTAAGTCCACTCCGCA CATGTGGAAAAGCTCCCACAGTGCCCCCTCCACTTTCATAATCAGGCAGACCTTCATAATAGAAACAGATCCTGCTG CTCCACCACCTGCAGCGTGTTCAAAACAACAAGATTCAATAAGGTTCTGCCCTCCGCCCTGAGCTCGCGCCTCAATG TCAGCTGCAAAAAGTCACTTAAGTCCTGGGCCACTACAGCTGACAATTCAGAGCCAGGGCTAAGCGTGGGACTGGCA AGCGTAAGGGAAAACTTTAATGCTCCAAAGCTAGCACCCAAAAACTGCATGCTGGAATAAGCTCTCTTTGTGTCTCC GGTGATGCCTTCCAAAATGTGAGTGATAAAGCGTGGTAGTTTTTCTTTAATCATTTGCGTAATAGAAAAGTCCTCTA AATAAGTCACTAGGACCCCAGGGACCACAATGTGGTAGCTTACACCGCGTCGCTGAAGCATGGTTAGTAGAGATGAG AGTCTGAAAAACAGAAAGCATGCACTAAACTAAGGTGGCTATTTTCACTGAAGGAAAAATCACTCTCTCCAGCAGCA GGGTACCCACTGGGTGGCCCTTGCGGACATACAAAAATCGGTCCGTGTGATTAAAAAGCAGCACAGTAAGTTCCTGT CTTCTTCCGGCAAAAATCACATCAGACTGGGTTAGTATGTCCCTGGCATGGTAGTCATTCAAGGCCATAAATCTGCC CTGATATCCAGTAGGAACCAGCACACTCACTTTTAGGTGAAGCAATACCACCCCATGCGGAGGAATGTGGAAAGATT CAGGGCAAAAAAATTATATCTATTGCTAGCCCCTTCCTGGACGGGAGCAATCCCTCCAGGACTATCTATAAAAGCAT ACAGAGATTCAGCCATAGCTTAGCCCGCTTACCAGTAGACAGAAAGCACAGCAGTACAAGCGCCAACAGCAGCAACT GACTACCCACTGACCCAGCTCCCTATTTAAAGGCACCTTACACTGACGTAATGACCAAAGGTCTAAAAACCCCGCCA AAAAAAACACACACGCCCTGGGTGTTTTTCACAAAAACACTTCCGCGTTCTCACTTCCTCGTATCGATTTTGTGACT CAACTTCCGGGTTCCCACGTTACGTCACTTCTGCCCTTACATGTAACTTGGCCGTATGGCGCCATCTTGCCCACGTC CAAAATGGCTTTCATGACCGGCCACGCCTCCGCGCCGGCCGTTAGCCGTGCGTCGTGACGTTATTTGCATCACCGCT TCTCGTCCAATCAGCGTTGGCTCCGCCCCAAAACCGTTAAAATTCAAAAGCTCATTTGCATATTAACTTTTGTTTAC TTTGTGGGGTATATTATGATGATG

GenBank Accession No. ABK59080 MSKRLRVEDDENPVYPYGYARNQNIPFLTPPFVSSDGFKNFPPGVLSLKLADPITITNGDVSLKVGGGLTLQDGSLT VNPKAPLQVNTDKKLELAYDNPFESSANKLSLKVGHGLKVLDEKSAAGLKDLIGKLVVLTGKGIGTENLENTDGSSR GIGINVRAREGLTFDNDGYLVAWNPKYDTRTLWTTPDTSPNCTIAQDKDSKLTLVLTKCGSQILANVSLIVVAGKYH IINNKTNPKIKSFTIKLLFNKNGVLLDNSNLGKAYWNFRSGNSNVSTAYEKAIGFMPNLVAYPKPSNSKKYARDIVY GTIYLGGKPDQPAVIKTTFNQETGCEYSITENFSWSKTYENVEFETTSFTFSYIAQE

GenBank Accession No. ABK59086 MRRAVVSSSPPPSYESVMAQATLEVPFVPPRYMAPTEGRNSIRYSELAPLYDTTRVYLVDNKSADIASLNYQNDHSN FLTTVVONNDFTPAEASTQTINFDERSRWGGDLKTILHTNMPNVNEYMFTSKFKARVMVARKKAEGADANDRSKDIL EYQWFEFTLPEGNFSETMTIDLMNNAILENYLOVGRONGVLESDIGVKFDSRNFKLGWDPVTKLVMPGVYTYEAFHP DVVLLPGCGVDFTESRLSNLLGIRKKOPFQEGFRIMYEDLVGGNIPALLNVKEYLKDKEEAGKADANTIKAQNDAVP RGDNYASAAEAKAAGKEIELKAILKDDSDRSYNVIEGTTDTLYRSWYLSYTYGDPEKGVQSWTLLTTPDVTCGAEQV YWSLPDLMQDPVTFRSTQQVSNYPVVGAELMPFRAKSFYNDLAVYSQLIRSYTSLTHVENRFPDNQILCRPPAPTIT TVSENVPALTDHGTLPLRSSIRGVQRVTVTDARRRTCPYVYKALGIVAPRVLSSRTF

GenBank Accession No. ABK59070 MCLTARERAKMATPSMMPQWAYMHIAGQDASEYLSPGLVQFARATDTYFSLGNKFRNPTVAPTHDVTTDRSQRLTLR FVPVDREDTTYSYKARFTLAVGDNRVLDMASTYFDIRGVLDRGPSFKPYSGTAYNSLAPKGAPNPSQWTTKEKONGG TGAEKDVTKTFGLAAMGGSNISKDGLQIGTDKTANAEKPIYADKTFQPEPQVGEENWODNDEYYGGRALKKDTKMKP CYGSFAKPTNKEGGQAKLKETPNGTDPQYDVDMAFFDSSTINIPDVVLYTENVDLETPDTHVVYKPGKEDDSSEANL TQQSMPNRPNYIGFRDNFVGLLYYNSTGNMGVLAGQASQLNAVVDLQDRNTELSYQLLLDSLGDRTRYFSMWNSAVD SYDPDVRIIENHGVEDELPNYCFPLDGVQTNSAYQGVKLKPDQTGGGVNGDWVKDDDISAHNQIGKGNIFAMEINLQ ANLWKSFLYSNVALYLPDSYKYTPANVTLPANTNTYEYMNGRVVAPSLVDAYINIGARWSLDPMDNVNPFNHHRNAG LRYRSMLLGNGRYVPFHIQVPQKFFAIKNLLLLPGSYTYEWNFRKDVNMILQSSLGNDLRVDGASVREDSVNLYATF FPMAHNTASTLEAMLRNDTNDOSENDYLSAANMLYPIPAKATNVPISIPSRNWAAFRGWSFTRLKTKETPSLGSGFD PYFVYSGSIPYLDGTFYLNHTFKKVSIMFDSSVSWPGNDRLLTPNEFEIKRSVDGEGYNVAQCNMTKDWFLVOMLSH YNIGYQGFHVPEGYKDRMYSFFRNFQPMSRQVVDEINYKDYKAVTLPFOHNNSGFTGYLAPTMRQGQPYPANFPYPL IGSTAVPSVTQKKFLCDRVMWRIPFSSNFMSMGALTDLGQNMLYANSAHALDMTFEVDPMDEPTLLYLLFEVFDVVR VHQPHRGVIEAVYLRTPFSAGNATT

GenBank Accession No. AY737798 (SEQ ID NO: 218) CAATCAATATAATATACCTTATAGATGGAATGGTGCCAATATGTAAATGAGGTAATTTAAAAAAGTGCGCGCTGTGT GGTGATTGGCTGCGGGGTGAACGGCTAAAAGGGGCGGACATGCTGGGAGGTGACGTGACTTATGGGGGAGGAGTTAT GTTGCAAGTTATCGCGGTAAAGGTGACGTAAAACGAGGTGTGGTTTGGACACGGAAGTAGACAGTTTTCCCACGCTT ACTGACAGGATATGAGGTAGTTTTGGGCGGATGCAAGTGAAAATTCTCCATTTTCGCGCGAAAACTGAATGAGGAAG TGAATTTCTGAGTCATTTCGCGGTTATGACAGGGTGGAGTATTTGCCGAGGGCCGAGTAGACTTTGACCGTTTACGT GGAGGTTTCGATTACCGTGTTTTTCACCTAAATTTCCGCGTACGGTGTCAAAGTCCTGTGTTTTTACGTAGGTGTCA GCTGATCGCTAGGGTATTTAAACCTGTCGAGTTCCGTCAAGAGGCCACTCTTGAGTGCCAGCGAGAAGAGTTTTCTC CTCCGCGCCGCGAGTCAGTTTTGCGCTTTGAAAATGAGACACCTGCGATTCCTGCCACAGGAGATTATCTCCAGCGA GACCGGGATAGAAATACTGGAGTTTGTGGTAAATACCCTGATGGGAGATGACCCGGAACCGCCAGCGCAGCCTTTCG ATCCACCTACGCTGCACGATCTGTATGATTTAGAGGTAGACGGGCCGGACGATCCCAATGAGGAAGCTGTAAATGGG TTTTTTACTGATTCTATGCTACTAGCTGCCGATGAAGGATTGGACATAAACCCTCCTCCTGAGACCCTTGATACCCC AGGGGTGGTTGTGGAAAGCGGCAGAGGTGGGATAAAATTGCCTGATCTGGGAGCAGCTGAAATGGACTTGCGTTGTT ATGAAGAGGGTTTTCCTCCGAGTGATGATGAAGATGGGGAAACTGAACAGTCCATCCATACCGCAGTGAATGAGGGA GTAAAAGCTGCCAGCGATGTTTTTAAGTTGGACTGTCCGGAGCTGCCTGGACATGGCTGTAAGTCTTGTGAATTTCA CAGGAATAACACTGGAATGAAAGAACTATTGTGCTCGCTTTGCTATATGAGAATGCACTGCCATTTTATTTACAGTA AGTGTATTTAAGTGAAATTTAAAGGAATAGTGTAGCTGTTTAATAACTGTTGAATGGTAGATTTATGTTTTTACTTG TGATTTTTTGTAGGTCCTGTGTCTGATGATGAGTCGCCTTCTCCTGATTCAACTACCTCACCTCCTGAAATTCAGGC GCCCGTACCTGCAAACGTATGCAAGCCCATTCCTGTGAAGCCTAAGTCTGGGAAACGCCCTGCTGTGGATAAGCTTG AGGACTTGTTGGAGGGTGGGGATGGACCTTTGGACCTTAGTACCCGGAAACTGCCAAGGCAATGAGTGCCCTGCAGC TGTGTTTATTTAATGTGACGTCATGTAATAAAATTATGTCAGCTGCTGAGTGTTTTATTGCTTCTTGGGTGGGGACT TGGATATATAAGTAGGAGCAGATCTGTGTGGTTAGCTTATAGCAACCTGCTGCCATCCATGGAGGTTTGGGCTATCT TGGAAGACCTGAGACAGACTAGGCTACTGCTAGAAAACGCCTCGGACGGAGTCTCTGGCTTTTGGAGATTCTGGTTC GGTGGTGATCTAGCTAGGCTAGTGTTTAGGATAAAACAGGACTACAGGGAAGAATTTGAAAAGTTATTGGACGACAG TCCAGGACTTTTTGAAGCTCTTAACTTGGGCCATCAGGCTCATTTTAAGGAGAAGGTTTTATCAGTTTTAGATTTTT CTACTCCTGGTAGAACTGCTGCTGCTGTAGCTTTTCTTACTTTTATATTGGATAAATGGATCCGACAAACCCACTTC AGCAAGGGATACGTTTTGGATTTCATAGCAGCAGCTTTGTGGAGAACATGGAAGGCTCGCAGCATGAGGACAATCTT AGATTACTGGCCAGTGCAGCCTCTGGGAGTAGCAGGGATACTGAGACACCCACCGACCATGCCAGCGGTTCTGGAGG AGGAGCAGCAGGAGGACAATCCGAGAGCCGGCCTGGACCCTCCGGTGGAGGAGTAGCTGACCTGTTTCCTGAACTGC GACGGGTGCTTACTAGGTCTACGTCCAGTGGACAGGACAGGGGCATTAAGAGGGAAAGGAATCCTAGTGGGAATAAT TCAAGAACCGAGTTGGCTTTAAGTTTAATGAGCCGTAGGCGTCCTGAAACTGTTTGGTGGCATGAGGTTCAGAGCGA AGGCAGGGATGAAGTTTCAATATTGCAGGAGAAATATTCACTAGAACAACTTAAGACCTGTTGGTTGGAACCTGAGG ATGATTGGGAGGTGGCCATTAGGAATTATGCTAAGATATCTCTGAGGCCTGATAAACAGTATAGAATTACTAAAAAG ATTAATATTAGAAATGCATGCTACATATCAGGGAATGGGGCAGAGGTTATAATAGATACCCAAGATAAAGCAGCTTT TAGATGTTGTATGATGGGTATGTGGCCAGGGGTTGTCGGCATGGAAGCAGTAACATTTATGAATATTAGGTTTAAAG GGGATGGGTATAATGGCATTGTATTTATGGCTAACACTAAGCTGATTCTACATGGTTGTAGCTTTTTTGGGTTTAAT AATACTTGTGTAGAAGCTTGGGGGCAAGTTGGTGTGAGGGGTTGTAGTTTTTATGCATGCTGGATTGCAACATCAGG TAGGGTCAAGAGTCAGTTGTCTGTGAAGAAATGCATGTTTGAGAGATGTAATCTTGGCATACTGAATGAAGGTGAAG CAAGGGTCCGCCACTGCGCAGCTACAGAAACTGGCTGCTTCATTCTAATAAAGGGAAATGCCAGTGTGAAGCATAAT ATGATCTGTGGACATTCGAATGAGAGGCCTTATCAGATGCTGACTTGCGCTGGTGGACATTGCAATATTCTTGCTAC CGTGCATATCGTTTCCCATGCACGCAAGAAATGGCCTGTATTTGAACATAATGTGATTACCAAGTGCACCATGCACA TAGGTGGTCGCAGGGGAATGTTTATGCCTTACCAGTGTAACATGAATCATGTGAAGGTGATGTTGGAACCAGATGCC TTTTCCAGAGTGAGCTTAACAGGAATCTTTGATATGAATATTCAACTATGGAAGATACTGAGATATGATGACACTAA ACCGAGGGTGCGCGCATGCGAATGCGGAGGCAAGCATGCTAGATTCCAGCCGGTGTGCGTGGATGTGACTGAAGACC TGAGACCCGATCATTTGGTGCTTGCCTGCACTGGAGCGGAGTTCGGTTCTAGTGGTGAAGAAACTGACTAAAGTGAG TAGTGGGGCAAGATGTGGATGGAGACTTTCAGGTTGGTAAGGTGGACAGATTGGGTAAATTTTGTTAATTTCTGTCT TGCAGCTGCCATGAGTGGAAGCGCTTCTTTTGAGGGGGGAGTATTTAGCCCTTATCTGACGGGCAGGCTCCCATCAT GGGCAGGAGTTCGTCAGAATGTCATGGGATCCACTGTGGATGGGAGACCCGTCCAGCCCGCCAATTCCTCAACGCTG ACCTATGCCACTTTGAGTTCGTCACCATTGGATGCAGCTGCAGCCGCCGCCGCTACTGCTGCCGCCAACACCATCCT TGGAATGGGCTATTACGGAAGCATCGTTGCCAATTCCAGTTCCTCTAATAACCCTTCAACCCTGGCTGAGGACAAGC TACTTGTTCTCTTGGCTCAGCTCGAGGCCTTAACCCAACGCTTAGGCGAACTGTCTAAGCAGGTGGCCCAGTTGCGT GAGCAAACTGAGTCTGCTGTTGCCACAGCAAAGTCTAAATAAAGATCTCAAATCAATAAATAAAGAAATACTTGTTA TAAAAACAAATGAATGTTTATTTGGTTTTTCGCGCGCGGTATGCCCTGGACCATCGGTCTCGATCATTGAGAACTCG GTGGATCTTTTCCAGTACCCTGTAAAGGTGGGATTGAATGTTTAGATACATGGGCATTAGTCCGTCTCGGGGGTGGA GATAGCTCCATTGAAGAGCCTCTTGCTCCGGGGTAGTGTTATAAATCACCCAGTCATAGCAAGGTCGGAGTGCATGG TGTTGCACAATATCTTTTAGGAGCAGACTAATTGCAACGGGGAGGCCCTTAGTGTAGGTGTTTACAAATCTGTTGAG CTGGGACGGGTGCATCCTGGGGGAAATTATATGCATTTTGGACTGGATCTTGAGGTTGGCAATGTTGCCGCCTAGAT CCCGTCTCGGGTTCATATTGTGCAGAACCACCAAGACAGTGTATCCGGTGCACTTGGGAAATTTATCATGCAGCTTA GAGGGAAAAGCATGAAAAAATTTGGAGACGCCTTTGTGACCCCCCAGATTCTCCATGCACTCATCCATAATGATAGC GATGGGGCCGTGGGCAGCGGCACGGGCGAACACGTTCCGGGGGTCTGAAACATCATAGTTATGCTCCTGAGTCAGGT CATCATAAGCCATTTTAATAAACTTTGGGCGGAGGGTGCCAGATTGGGGGATGAAAGTTCCCTCTGGCCCGGGAGCA TAGTTTCCCTCACATATTTGCATTTCCCAGGCTTTCAGTTCCGAGGGGGGGATCATGTCCACCTGCGGGGCTATAAA AAATACCGTTTCTGGAGCCGGGGTGATTAACTGGGATGAGAGCAAATTCCTAAGCAGCTGAGACTTGCCGCACCCAG TGGGACCGTAAATGACCCCAATTACGGGTTGCAGATGGTAGTTTAGGGAGCGACAGCTGCCGTCCTCCCGGAGCAGG GGGGCCACTTCGTTCATCATTTCCCTTACATGGATATTTTCCCGCACCAAGTCCGTTAGGAGGCGCTCTCCCCCAAG GGATAGAAGCTCCTGGAGCGAGGAGAAGTTTTTCAACGGTTTCAGCCCGTCAGCCATGGGCATTTTGGAAAGAGTCT GTTGCAAGAGCTCGAGCCGGTCCCAGAGCTCGGTGATGTGCTCTATGGCATCTCGATCCAGCAGACCTCCTCGTTTC GCGGGTTGGGACGGCTCCTGGAGTAGGGAATCAGACGATGAGCGTCCAGCGCTGCTAGGGTCCGATCCTTCCATGGT CGCAGCGTCCGAGTCAGGGTTGTTTCCGTCACGGTGAAGGGGTGCGCGCCTGGTTGGGCGCTTGCGAGGGTGCGCTT CAGACTCATCCTGCTGGTCGAGAACCGCTGCCGATCGGCGCCCTGCATGTCGGCCAGGTAGCAGTTTACCATGAGTT CGTAGTTGAGCGCCTCGGCCGCGTGGCCTTTGGCACGGAGCTTACCTTTGGAAGTTTTATGGCAGGCGGGGCAGTAG ATACATTTGAGGGCATACAGCTTGGGCGCGAGGAAAATGGATTCGGGGGAGTATGCATCCGCACCGCAGGAGGCGCA GACGGTTTCGCACTCCACGAGCCAGGTCAGATCCGGCTCATCGGGGTCAAAAACAAGTTTTCCGCCATGTTTTTTGA TGCGTTTCTTACCTTTGGTTTCCATGAGTTCGTGTCCCCGCTGGGTGACAAAGAGGCTGTCCGTGTCCCCGTAGACC GACTTTATGGGCCTGTCCTCGAGCGGAGTGCCTCGGTCCTCTTCGTAGAGGAACCCAGCCCACTCTGATACAAAAGC GCGTGTCCAGGCCAGCACAAAGGAGGCCACGTGGGAGGGGTAGCGGTCGTTGTCAACCAGGGGGTCCACCTTCTCTA CGGTATGTAAACACATGTCCCCCTCCTCCACATCCAAGAATGTGATTGGCTTGTAAGTGTAGGCCACGTGACCAGGG GTCCCCGCCGGGGGGGTATAAAAGTGGGCGGGCCTCTGTTCGTCCTCACTGTCTTCCGGATCACTGTCCAGGAGCGC CAGCTGTTGGGGTAGGTATTCTCTCTCGAAGGCGGGCATGACCTCTGCACTCAGGTTGTCAGTTTCTAGGAACGAGG AGGATTTGATATTGACAGTACCAGCAGAGATGCCTTTCATAAGACTCTCGTCCATCTGGTCAGAAAACACAATCTTC TTGTTATCCAGCTTGGTGGCAAATGATCCATAGAGGGCATTGGATAGAAGCTTGGCGATGGAGCGCATGGTTTGGTT CTTTTCCTTGTCCGCGCGCTCCTTGGCGGCGATGTTAAGCTGGACGTACTCGCGCGCCACACATTTCCATTCAGGGA AGATGGTTGTCAGTTCATCCGGAACTATTCTGACTCGCCATCCCCTATTGTGCAGGGTTATCAGATCCACACTGGTG GCTACCTCGCCTCGGAGGGGCTCATTGGTCCAGCAGAGTCGACCTCCTTTTCTTGAACAGAAAGGGGGGAGGGGGTC TAGCATGAACTCATCAGGGGGGTCCGCATCTATGGTAAATATTCCCGGTAGCAAATCTTTGTCAAAATAGCTGATGG TGGCGGGATCATCCAAGGTCATCTGCCATTCTCGAACTGCCAGCGCGCGCTCGTAGGGGTTAAGAGGGGTGCCCCAG GGCATGGGGTGGGTGAGCGCGGAGGCATACATGCCACAGATATCGTAGACATAGAGGGGCTCTTCGAGGATGCCGAT GTAAGTGGGATAACAGCGCCCCCCTCTGATGCTTGCTCGCACATAGTCATAGAGTTCATGTGAGGGGGCGAGAAGAC CCGGGCCCAGATTGGTGCGGTTGGGTTTTTCCGCCCTGTAAACGATCTGGCGAAAGATGGCATGGGAATTGGAAGAG ATAGTAGGTCTCTGGAATATGTTAAAATGGGCATGAGGTAGGCCTACAGAGTCCCTTATGAAGTGGGCATATGACTC TTGCAGCTTGGCTACCAGCTCGGCGGTGACGAGTACGTCCAGGGCACAGTAGTCGAGAGTTTCCTGGATGATGTCAT AACGCGGTTGGCTTTTCTTTTCCCACAGCTCGCGGTTGAGAAGGTATTCTTCGCGATCCTTCCAGTACTCTTCGAGG GGAAACCCGTCTTTTTCTGCACGGTAAGAGCCCAACATGTAGAACTGATTGACTGCCTTGTAGGGACAGCATCCCTT CTCCACTGGGAGAGAGTATGCTTGGGCTGCATTGCGCAGCGAGGTATGAGTGAGGGCAAAAGTGTCCCTGACCATGA CTTTGAGGAATTGATACTTGAAGTCGATGTCATCACAGGCCCCCTGTTCCCAGAGTTGGAAGTCCACCCGCTTCTTG TAGGCGGGGTTGGGCAAAGCGAAAGTAACATCATTGAAGAGGATCTTGCCGGCCCTGGGCATGAAATTTCGGGTGAT TCTGAAAGGCTGAGGGACCTCTGCTCGGTTATTGATAACCTGAGCGGCCAAGACGATCTCATCAAAGCCATTGATGT TGTGCCCCACTATGTACAGTTCTAAGAATCGAGGGGTGCCCCTGACATGAGGCAGCTTCTTGAGTTCTTCAAAAGTG AGGTCTGTAGGGTCAGTGAGAGCATAGTGTTCGAGGGCCCATTCGTGCACGTGAGGGTTCGCTTTGAGGAAGGAGGA CCAGAGGTCCACTGCCAGTGCTGTTTGTAACTGGTCCCGGTACTGACGAAAATGCTGCCCGACTGCCATCTTTTCTG GGGTGACGCAATAGAAGGTTTGGGGGTCCTGCCGCCAGCGATCCCACTTGAGTTTTATGGCCAGGTCATAGGCGATG TTGACGAGCCGCTGGTCTCCAGAGAGTTTCATGACCAGCATGAAGGGGATTAGCTGCTTGCCAAAGGACCCCATCCA GGTGTAGGTTTCCACATCGTAGGTGAGGAAGAGCCTTTCTGTGCGAGGATGAGAGCCAATCGGGAAGAACTGGATCT CCTGCCACCAGTTGGAGGAATGGCTGTTGATGTGATGGAAGTAGAACTCCCTGCGACGCGCCGAGCATTCATGCTTG TGCTTGTACAGACGGCCGCAGTACTCGCATCGATTCACGGGATGCACCTCATGAATGAGTTGTACCTGACTTCCTTT GACGAGAAATTTCAGTGGAAAATTGAGGCCTGGCGATTGTACCTCGCGCTCTACTATGTTGTCTGCATCGGCATGAC CATCTTCTGTCTCGATGGTGGTCATGCTGACGAGCCCTCGCGGGAGGCAAGTCCAGACCTCGGCGCGGCAGGGGCGG AGCTCGAGGACGAGAGCGCGCAGGCCGGAGCTGTCCAGGGTCCTGAGACGCTGCGGAGTCAGGTTAGTAGGCAGTGT CAGGAGATTGACTTGCATGATCTTTTCGAGGGCGTGAGGGAGGTTCAGATGGTACTTGATCTCCACGGGTCCGTTGG TGGAGATGTCGATGGCTTGCAGGGTTCCGTGCCCCTTGGGCGCTACCACCGTGCCCTTGTTTTTCCTTTTGGGCGGC GGTGGCTCTGTTGCTTCTTGCATGTTTAGAAGCGGTGTCGAGGGCGCGCACCGGGCGGCAGGGGCGGTTCGGGACCC GGCGGCATGGCCGGCAGTGGTACGTCGGCGCCGCGCGCGGGTAGGTTCTGGTACTGCGCCCTCAGAAGACTCGCATG CGCCACGACGCGGCGGTTGACATCCTGGATCTGACGCCTCTGGGTGAAAGCTACCGGCCCCGTGAGCTTGAACCTGA AAGAGAGTTCAACAGAATCAATCTCGGTATCGTTGACGGCGGCTTGCCTAAGGATTTCTTGCACGTCGCCAGAGTTG TCCTGGTAGGCGATCTCGGCCATGAACTGCTCGATCTCTTCCTCTTGAAGATCTCCGCGGCCCGCTCTCTCGACGGT GGCCGCGAGGTCGTTGGAGATACGCCCAATGAGTTGAGAGAATGCATTCATGCCCGCCTCGTTCCAGACGCGGCTGT AGACCACAGCCCCCACGGGATCTCTCGCGCGCATGACCACCTGGGCGAGGTTGAGCTCCACGTGGCGGGTGAAGACC GCATAGTTGCATAGGCGCTGGAAAAGGTAGTTGAGTGTGGTGGCGATGTGCTCGGTGACGAAGAAATACATGATCCA TCGTCTCAGCGGCATCTCGCTGACATCGCCCAGCGCTTCCAAGCGCTCCATGGCCTCGTAGAAGTCCACGGCAAAGT TGAAAAACTGGGAGTTACGCGCGGACACGGTCAACTCTTCTTCCAAAAGACGGATGAGTTCGGCGATGGTGGTGCGC ACCTCGAGCTCGAAAGCCCCTGGGATTTCTTCCTCAATCTCTTCTTCTTCCACTAACATCTCTTCCTCTTCAGGTGG GGCTGCAGGAGGAGGGGGAACGCGGCGACGCCGGCGGCGCACGGGCAGACGGTCGATGAATCTTTCAATGACCTCTC CGCGGCGGCGGCGCATGGTCTCGGTGACGGCACGACCGTTCTCCCTGGGTCTCAGAGTGAAGACGCCTCCGCGCATC TCCCTGAAGTGGTGACTGGGAGGCTCTCCGTTGGGCAGGGACACCGCGCTGATTATGCATTTTATTAATTGCCCCGT AGGTACTCCGCGCAAGGACCTGATCGTCTCAAGATCCACGGGATCTGAAAACCTTTCGACGAAAGCGTCTAACCAGT CGCAATCGCAAGGTAGGCTGAGCACTGTTTCTTGCGGGGGGGGGGGGCTAGACGCTCGGTCGGGGTTCTCTCTTTCT TCTCCTTCCTCCTCTTTGGAGGGTGAGACGATGCTGCTGGTGATGAAATTAAAATAGGCAGTTTTGAGACGGCGGAT GGTGGCGAGGAGCACCAGGTCTTTGGGTCCGGCTTGTTGGATGCGCAGGCGATGGGCCATTCCCCAAGCATTATCCT GACATCTGGCCAGATCTTTATAGTAGTCTTGCATGAGTCGTTCCACGGGCACTTCTTCTTCGCCCGCTCTGCCATGC ATGCGAGTGATCCCGAACCCGCGCATGGGCTGGACAAGTGCCAGGTCCGCTACAACCCTTTCTGCGAGGATGGCTTG CTGCACCTGGGTGAGGGTGGCTTGGAAGTCGTCAAAGTCCACGAAGCGGTGGTAAGCCCCGGTGTTGATTGTGTAGG AGCAGTTGGCCATGACTGACCAGTTGACTGTCTGGTGCCCAGGGCGCACAAGCTCGGTATACTTAAGGCGCGAGTAT GCGCGGGTGTCAAAGATGTAATCGTTACAGGTGCGCACCAGGTACTGGTAGCCGATGAGAAAGTGCGGCGGCGGCTG GCGGTATAGGGGCCATCGCTCTGTAGCCGGGGCGCCAGGGGCGAGGTCTTCCAGCATGAGGCGGTGATAACCGTAGA TGTACCTGGACATCCAGGTGATACCGGAGGCGGTGGTGGATGCCCGCGGGAACTCGCGTACGCGGTTCCAGATGTTG CGCAGCGGCATGAAGTAGTTCATGGTAGGCACGGTTTGGCCCGTGAGACGTGCACAGTCGTTGATGCTCTAGACATA CGGGCAAAAACGAAAGCGGTCAGCGGCTCGTCTCCGTGGCCTGGAGGCTAAGCGAACGGGTTGGGCTGCGCGTGTAC CCCGGTTCGAATCTCGGATCAGGCTGGAGCCGCAGCTAACGTGGTACTGGCACTCCCGTCTCGACCCAGGCCTGCAC AAAACCTCCAGGATACGGAGGCGGGTCGTTTTTTTTTTTTTGGCTTTTTCCTGGATGGGAGCCAATGCTGCGTCAAG CTTTAGAACACTCAGTTCTCGGGGCTGGGAGTGGCTCGCGCCCGTAGTCTGGAGAATCAATCGCCAGGGTTGCGTTG CGGTGTGCCCCGGTTCGAGTCTTAGCGCGCCGGATCGGCCGGTTTCCGCGACAAGCGAGGGTTTGGCAGCCTCGTCA TTTCTAAGACCCCGCCAGCCGACTTCTCCAGTTTACGGGAGCGAGCCCTCTTTTTTTGTTTTTTTGTTGCCCAGATG CATCCCGTGCTGCGACAGATGCGCCCCCAGCAACAGCCCCCTTCTCAGCAGCAGCTACAACAACAGCCACAAAAGGC TCTTCCTGCTCCTGTAACTACTGCGGCTGCAGCCGTCAGCGGCGCGGGGCAGCCCGCCTATGATCTGGACTTGGAAG AGGGCGAGGGACTGGCGCGCCTGGGCGCACCATCGCCCGAGCGGCACCCGCGGGTGCAACTGAAAAAGGACTCTCGC GAGGCGTACGTGCCCCAGCAGAACCTGTTCAGGGACAGGAGCGGCGAGGAGCCTGAGGAAATGCGAGCTTCCCGCTT TAACGCGGGTCGCGAACTGCGTCACGGTCTGGACCGAAGACGGGTGCTGCGTGATGATGATTTTGAAGTCGATGAAG TGACAGGAATAAGTCCTGCTAGGGCACATGTGGCCGCGGCCAACCTAGTATCAGCTTACGAGCAGACCGTGAAGGAG GAGCGCAACTTTCAAAAATCTTTCAACAACCATGTGCGCACCCTGATTGCCCGCGAGGAAGTGACACTGGGTCTGAT GCACCTGTGGGACCTGATGGAAGCCATTACCCAGAACCCCACCAGCAAACCTCTAACCGCTCAGCTGTTTCTGGTGG TGCAACATAGTAGAGACAATGAGGCATTTAGGGAGGCGCTGTTGAACATTACTGAGCCCGAGGGGAGATGGTTGTAT GATCTTATCAATATTCTGCAAAGTATAATCGTGCAAGAACGTAGCCTGGGTCTAGCTGAGAAGGTGGCTGCTATTAA CTACTCGGTCTTGAGCCTGGGCAAGCACTACGCTCGCAAGATCTACAAAACCCCATACGTACCTATAGACAAGGAGG TGAAGATAGATGGGTTTTATATGCGCATGACTCTCAAGGTGCTGACCTTGAGTGACGATCTGGGAGTGTACCGCAAC GACAGGATGCACCGCGCAGTGAGCGCCAGCAGAAGGCGTGAGCTGAGCGACAGAGAACTTATGCACAGCTTGCAAAG AGCTCTGACTGGGGCTGGAACCGAGGGGGAAAACTACTTTGACATGGGAGCGGACTTGCAGTGGCAGCCCAGTCGCA GGGCCCTGGACGCAGCAGGGTATGAGCTTCCTTACATAGAAGAGGTGGATGAAGGCCAGGATGAGGAGGGCGAGTAT CTGGAAGACTGATGGCGCGACCATCCATATTTTTGCTAGATGGAACAGCAGGCACCGGACCCCGCAAAACGGGCGGC GCTACAGAGCCAGCCGTCCGGCATTAACTCCTCGGACGATTGGAGCCAGGCCATGCAACGCATCATGGCGCTGACGA CCCGCAACCCCGAAGCCTTTAGACAGCAACCCCAGGCCAACCGCCTTTCTGCCATCCTGGAGGCCGTAGTGCCCTCC CGCTCCAACCCCACACACGAGAAGGTCCTGGCCATCGTGAACGCGCTGGTGGAGAACAAAGCCATACGTCCCGATGA GGCTGGGCTGGTATACAATGCCCTATTGGAGCGCGTAGCCCGTTACAACAGCAGCAACGTGCAGACCAACCTGGACC GGATGGTGACCGATGTGCGCGAGGCCGTGTCTCAGCGCGAGCGGTTCCAGCGAGACGCCAATTTAGGGTCGCTGGTG GCTTTGAACGCCTTCCTCAGCACTCAGCCTGCCAACGTGCCTCGCGGTCAGCAAGACTACACAAACTTTCTAAGTGC ATTGAGACTCATGGTGGCCGAAGTCCCTCAAAGCGAAGTGTACCAGTCCGGGCCAGACTACTTTTTCCAGACCAGCA GACAGGGCTTGCAGACAGTGAACCTGAGCCAGGCTTTTAAGAACCTGAATGGTCTGTGGGGAGTGCGCGCCCCAGTG GGAGATCGGGCGACCGTGTCTAGCTTGCTGACCCCCAACTCCCGCCTACTACTGCTCTTGGTAGCCCCATTCACTGA CAGCGGTAGCATCGACCGTAATTCGTACTTGGGCTATCTGTTGAACCTGTATCGCGAGGCCATAGGGCAAACTCAGG TAGATGAGCAAACCTATCAAGAAATTACCCAAGTGAGCCGCGCTCTGGGTCGGGAGGACACTGGCAGCTTGGAAGCC ACCTTAAACTTCTTGCTGACCAACCGGTCGCAGAAGATCCCTCCTCAGTATTCGCTTACCGCGGAGGAGGAACGGAT CCTGAGATACGTGCAGCAGAGCGTGGGACTGTTCCTAATGCAGGAGGGGGCGACTCCTACTGCTGCGCTCGATATGA CAGCCCGAAACATGGAGCCCAGCATGTATGCCAGTAACCGGCCTTTTATCAATAAACTGCTAGACTACTTACACAGG GCGGCTGCTATGAACTCTGATTATTTCACCAATGCTATCCTGAACCCCCATTGGCTGCCCCCACCTGGGTTCTATAC GGGCGAGTATGACATGCCCGACCCCAATGACGGGTTTTTATGGGACGATGTGGACAGTAGTGTTTTCTCCCCGCCTC CTGGTTATAACACTTGGAAGAAGGAAGGTGGCGATAGAAGGCACTCTTCCGTGTCACTGTCCGGAGCAACGGGTGCT GCAGCGGCTCCCGAGGCCGCAAGTCCTTTCCCTAGTTTGCCATTTTCGCTAAACAGTGTACGCAGCAGTGAGCTGGG AAGAATAACCCGTCCTCGCTTGATCGGCGAGGAGGAGTATTTGAACGACTCCCTGTTGAGACCCGAGAGGGAGAAGA ATTTCCCCAACAACGGGATAGAAAGCTTGGTTGACAAAATGAACCGCTGGAAGACGTACGCGCACGATCACAGGGAC GATCCCCGGGCGCTGGGGGATAGCCGGGGCATCGCTACCCGTAAACGCCAGTGGCACGACAGGCAGCGGGGCCTGGT GTGGGCCGATGATGATTCCGCCGACGACAGCAGCGTGTTGGACTTGGGTGGGAGTGGTGGTAACCCGTTCGCTCACC TGCGCCCCCGCGTCGGGCGCCTGATGTAAGAAACCGAAAATAAATACTCACCAAGGCCATGGCGACCAGCGTGCGTT CGTTTCTTCTCTGTTATAGCTAGTATGATGAGGCGAACCGTGCTAGGCGGAGCGGTGGTGTATCCGGAGGGTCCTCC TCCTTCGTACGAGAGCGTGATGCAGCAGGCGGCGGCGGCGGCGATGCAGCCACCACTGGAGGCTCCCTTTGTACCCC CTCGGTACCTGGCACCTACGGAGGGGAGAAACAGCATTCGTTACTCGGAGCTGGCACCATTGTATGATACCACCCGG TTGTATTTGGTGGACAACAAGTCCGCGGACATCGCCTCACTGAACTATCAGAACGACCACAGCAACTTCCTCACCAC GGTGGTGCAAAACAATGACTTTACCCCCACGGAGGCCAGCACCCAGACCATCAACTTTGACGAGCGGTCGCGATGGG GCGGTCAGCTGAAGACTATCATGCACACCAACATGCCCAACGTGAACGAGTACATGTTTAGCAACAAGTTCAAAGCT CGGGTGATGGTGTCTAGAAAGGCTCCTGAAGGTGTCACAGTAGATGACAATTATGATCACAAGCAGGATATTTTGGA ATATGAGTGGTTTGAGTTTACTCTACCGGAAGGGAACTTCTCAGCCACAATGACCATTGACCTAATGAACAATGCCA TCATTGATAATTACCTTGAAGTGGGCAGACAGAATGGAGTGTTGGAGAGTGACATTGGTGTTAAATTTGACACCAGG AACTTTAGACTGGGTTGGGATCCGGAAACTAAGTTGATTATGCCTGGGGTTTACACCTATGAGGCATTCCATCCTGA CATTGTATTGTTGCCTGGTTGCGGAGTTGACTTTACTGAAAGTCGCCTTAGTAACTTGCTTGGTATCAGGAAAAGAC ACCCATTCCAGGAGGGTTTTAAGATCTTGTATGAGGATCTTGAAGGGGGTAATATCCCGGCCCTGTTGGATGTAGAA GCCTATAAGAACAGTAAGAAAGAACGAGAAGCCAAAACAGAAGCCGCTAAAGCTGCTGCTATTGCTAAAGCCAACAT AGTTGTCAGCGACCCTGTAAGGGTGGCTAATGCCGAAGAAGTCAGAGGAGACAACTATACAGCTTCATCTGTTGCAA CTGAAGAATCGCTATTGGCTGCTGTGGCCGAAACTACAGAGACCAAACTCACTATTAAACCTGTAGAAAAAGACAGC AAGAGTAGAAGTTACAATGTCTTGGAAGATAAAGTGAATACAGCCTACCGCAGCTGGTACCTGTCCTACAACTATGG TGACCCTGAAAAAGGAGTCCGTTCCTGGACACTGCTCACCACCTCGGATGTCACCTGTGGAGCAGAGCAGGTGTACT GGTCGCTCCCAGACATGATGCAGGACCCTGTCACATTCCGTTCCACGAGACAAGTCAGCAACTATCCAGTGGTAGGT GCAGAGCTCATGCCGGTCTTCTCAAAGAGTTTCTACAACGAGCAAGCCGTGTACTCCCAGCAGCTTCGCCAGTCCAC CTCGCTCACGCACGTCTTCAACCGCTTCCCTGAGAACCAGATCCTCATCCGCCCGCCAGCGCCCACCATTACCACCG TCAGTGAAAACGTTCCTGCTCTCACAGATCACGGGACCCTGCCGTTGCGCAGCAGTATCCGGGGAGTCCAGCGCGTG ACCGTTACTGACGCCAGACGCCGCACCTGCCCCTACGTCTACAAGGCCCTGGGCATAGTCGCGCCGCGCGTCCTTTC AAGCCGCACTTTCTAAAAAAAAATGTCCATTCTTATCTCACCTAGTAATAACACCGGTTGGGGCCTGCGCGCGCCAA GCAAGATGTACGGAGGTGCTCGCAAACGCTCTACACAGCACCCTGTGCGCGTGCGCGGGCACTTCCGCGCTCCATGG GGCGCCCTCAAGGGTCGTACCCGCACTAGAACCACCGTCGATGATGTGATCGACCAGGTGGTGGCCGATGCTCGTAA TTATACTCCTACTGCACCTACATCTACTGTGGATGCAGTTATTGACAGCGTAGTGGCTGACGCCCGCGCCTATGCTC GCCGGAAGAGCAGGCGGAGACGCATCGCCAGGCGCCACCGGGCTACTCCCGCTATGCGAGCGGCAAGAGCTCTGCTG AGGAGGGCCAAACGCGTGGGGCGAAGAGCTATGCTTAGAGCGGCCAGACGCGCGGCTTCAGGTGCCAGTGCCGGCAG GTCCCGCAGGCGCGCAGCCACGGCGGCAGCAGCGGCCATTGCCAACATGGCCCAACCGCGAAGAGGCAATGTGTACT GGGTGCGCGACGCCACCACCGGCCAGCGCGTGCCCGTGCGCACCCGCCCCCCTCGCTCTTAGAAGATACTGAGCAGT CTCCGATGTTGTGTCCCAGCGAGGATGTCCAAGCGCAAATACAAGGAAGAGATGCTCCAGGTCATCGCGCCTGAAAT CTACGGTCCGCCGGTGAAGGATGAAAAAAAGCCCCGCAAAATCAAGCGGGTCAAAAAGGACAAAAAGGAAGAAGATG GCAATGATGGTCTGGCGGAGTTTGTACGCGAGTTCGCCCCAAGGCGGCGAGTGCAGTGGCGTGGACGCAAAGTGCAG CCTGTGCTGAGACCTGGAACCACGGTGGTCTTTACGCCCGGCGAGCGCTCCAGCACTGCTTTTAAGCGGTCCTATGA TGAGGTGTATGGGGATGATGATATTCTGGAGCAGGCGGCCGACCGCCTGGGCGAGTTTGCTTATGGCAAGCGCTCCC GCTCGAGCCCCAAGGAGGAGGCGGTGTCCATTCCCTTGGACAATGGGAATCCCACCCCTAGTCTCAAGCCAGTCACC CTGCAGCAAGTGCTGCCCGTGCCTCCACGCAGAGGCAACAAGCGAGAGGGTGAGGATCTGTATCCCACTATGCAATT GATGGTGCCCAAGCGCCAGCGGCTGGAGGACGTGCTGGAGAAAATGAAAGTGGATCCCGATATACAACCTGAGGTCA AAGTGAGACCCATCAAGCAGGTGGCGCCAGGTTTGGGAGTACAAACCGTAGACATCAAGATTCCCACCGAGTCAATG GAAGTCCAAACCGAACCTGCAAAGCCCACAACCACCTCCATTGAGGTGCAAACGGATCCCTGGATGACCGCACCCGT TACAACTCCAGCTGCTGTCAACACCACTCGAAGATCCCGGCGAAAGTACGGTCCAGCAAGTTTGCTGATGCCAAATT ATGCTCTGCACCCATCCATTATTCCAACTCCGGGTTACCGAGGCACTCGCTACTACCGCAGCAGGAGCAGCACTTCC CGCCGTCGCCGCAAAACACCTGCAAGTCGTAGTCATCGTCGTCGCCGCCGCCCCACCAGCAATCTGACTCCCGCTGC TCTGGTGCGGAGAGTGTATCGCGATGGCCGCGCGGATCCCCTGACGTTACCGCGCGTACGCTACCATCCAAGCATCA CAACTTAACAACTGTTGCCGCTGCCTCCTTGCAGATATGGCCCTCACTTGCCGCCTTCGTGTCCCCATTACTGGCTA CCGAGGAAGAAACTCGCGCCGTAGAAGAGGGATGTTGGGGCGCGGAATGCGACGCCACAGGCGGCGGCGCGCTATCA GCAAGAGGCTGGGGGGTGGCTTTCTGCCTGCTCTGATCCCCATCATAGCCGCGGCGATCGGGGCGATACCAGGCATA GCTTCCGTGGCGGTTCAGGCCTCGCAGCGCCACTGACATTGGAAAAACTTATAAATAAAACAGAATGGACTCTGATG CTCCTGGTCCTGTGACTATGTTTTTGTAGAGATGGAAGACATCAATTTTTCATCCCTGGCTCCGCGACACGGCACGA GGCCGTACATGGGCACCTGGAGCGACATCGGCACCAGCCAACTGAACGGGGGCGCCTTCAATTGGAGCAGTATCTGG AGCGGGCTTAAAAATTTTGGCTCTACCATAAAAACCTATGGGAACAAAGCTTGGAACAGCAGCACAGGGCAGGCATT GAAAAATAAGCTTAAAGAGCAAAACTTCCAACAGAAGGTGGTTGATGGAATCGCCTCTGGTATCAATGGGGTGGTGG ATCTGGCCAACCAGGCCGTGCAGAAACAGATAAACAGCCGCCTTGACCCGCCGCCGTCAGCCCCTGGTGAAATGGAA GTGGAGGAAGATCTCCCTCCCCTTGAAAAGCGGGGCGACAAGCGTCCGCGCCCCGATCTGGAGGAGACACTAGTCAC ACGCTCAGACGACCCGCCCTCCTACGAGGAGGCAGTGAAGCTTGGAATGCCCACCACCAGACCTGTAGCCCCCATGG CTACCGGGGTAATGAAACCTTCTCAGTCACACCGACCCGCTACCTTGGACTTGCCTCCCCCTGCTGTTGCAGCGCCT GCTCGCAAGCCTGTCGCTACCCCGAAGCCCACCACCGTACAGCCCGTCGCCGTAGCCAGACCGCGTCCTGGGGGCAC TCCACGTCCGAATGCAAACTGGCAGAGTACTCTGAACAGCATCGTGGGTCTGGGCGTGCAAAGTGTAAAGCGCCGTC GCTGCTTTTAAATTAAATATGGAGTAGCGCTTAACTTGCCTGTCTGTGTGTATGTGTCATCATCACGCCGCCGCCGC AGCAACAGCAGAGGAGCAAGGAAGAGGTCGCGCGCCGAGGCTGAGTTGATTTCAAGATGGCCACCCCATCGATGCTG CCCCAGTGGGCATACATGCACATCGCCGGACAGGATGCTTCGGAGTACCTGAGTCCGGGTCTGGTGCAGTTCGCCCG CGCCACAGACACCTACTTCAATCTGGGGAACAAGTTTAGGAACCCCACCGTGGCGCCCACCCATGATGTGACCACCG ACCGCAGTCAGCGGCTGATGCTGCGCTTTGTGCCCGTTGACCGGGAAGACAATACCTACGCATACAAAGTTCGATAC ACCTTGGCTGTGGGCGACAACAGAGTGCTGGATATGGCCAGCACTTTCTTTGACATTCGGGGTGTGTTGGATAGAGG TCCTAGCTTCAAGCCATATTCTGGCACTGCTTACAACTCATTGGCCCCTAAGGGCGCTCCCAATACATCTCAGTGGC TTAATAAGGGAGATGAAGAGGATGGGGAAGACGACCAACAAGCTACATACACTTTTGGCAATGCGCCAGTAAAAGCC GAAGCTGAAATTACAAAAGAAGGACTGCCAATAGGTTTGGAAGTTCCATCTGAAGGTGGCCCTAAACCCATTTATGC TGATAAACTGTATCAGCCAGAACCTCAGGTGGGAGAGGAATCTTGGACTGATACGGATGGCACAGATGAAAAATATG GAGGCAGAGCACTTAAACCTGAAACTAAAATGAAACCCTGCTACGGGTCTTTCGCTAAACCTACTAATGTTAAAGGC GGGCAGGCAAAAGTGAAGAAAGAAGAAGAAGGCAAGGTTGAATATGACATTGACATGAACTTTTTCGACCTAAGATC ACAAATGACTGGCCTCAAGCCTAAAATTGTAATGTATGCAGAAAATGTGGATCTAGAAACTCCTGACACTCATGTGG TGTACAAACCTGGAGCTTCAGATGCTAGCTCTCATGCAAACCTTGGTCAACAGTCCATGCCCAATAGACCTAACTAT ATTGGCTTCAGGGACAACTTCATCGGACTCATGTACTACAACAGTACTGGCAACATGGGAGTGCTGGCTGGACAAGC GTCTCAGCTAAATGCAGTGGTTGACTTGCAAGACAGAAACACAGAATTGTCATATCAACTCTTGCTTGATTCTCTGG GGGACAGAACCAGATATTTCAGTATGTGGAATCAAGCAGTGGATAGCTATGACCCAGATGTGCGTGTTATTGAGAAC CATGGTGTGGAAGATGAACTTCCTAACTATTGTTTTCCATTGGATGGTGTAGGTCCGCGAATAGACAGTTACAAGGG AATTGAGACAAATGGTGATGAAACCACTACTTGGAAAGATTTAGAGCCAAAGGGCATAAGTGAAATTGCTAAGGGAA ATCCGTTTGCCATGGAAATTAACCTCCAAGCTAATCTCTGGAGAAGTTTTCTTTATTCCAATGTGGCTCTGTATCTC CCAGACTCCTACAAATACACCCCAGCCAATGTCACTCTTCCAACTAACACCAACACTTATGACTACATGAATGGGCG GGTGGTTCCCCCATCCCTGGTGGATACCTACGTAAACATTGGCGCCAGATGGTCTTTGGATGCCATGGACAATGTCA ACCCCTTTAACCATCACCGCAACGCTGGCCTGCGATACCGGTCCATGCTTTTGGGCAATGGTCGTTACGTGCCTTTC CACATTCAAGTGCCTCAGAAATTCTTTGCTGTGAAGAACCTGCTGCTTCTACCCGGTTCTTACACCTACGAGTGGAA CTTCAGAAAGGATGTGAACATGGTCCTGCAGAGTTCCCTTGGTAATGATCTCCGGGTCGATGGTGCCAGCATAAGTT TTACCAGCATCAATCTCTATGCCACCTTCTTCCCCATGGCCCACAACACTGCCTCCACCCTTGAAGCCATGCTGCGC AATGACACCAATGATCAATCATTCAATGACTACCTTTCTGCTGCCAACATGCTCTACCCCATCCCGGCCAACGCTAC CAACGTTCCCATCTCCATTCCCTCTCGCAACTGGGCCGCCTTCAGAGGCTGGTCCTTCACCAGACTCAAAACCAAGG AGACTCCCTCTTTGGGATCAGGGTTCGATCCCTACTTTGTTTACTCTGGTTCTATACCCTACCTGGATGGTACCTTC TACCTTAACCACACTTTCAAGAAAGTCTCCATCATGTTTGACTCTTCAGTGAGCTGGCCTGGTAATGACAGATTGCT AAGTCCAAATGAGTTCGAAATCAAGCGCACAGTTGATGGGGAAGGCTACAATGTGGCCCAATGTAACATGACCAAAG ACTGGTTCCTGGTCCAGATGCTTGCCAACTACAACATTGGATACCAGGGCTTCTACGTTCCTGAGGGTTACAAGGAT CGCATGTACTCCTTCTTCAGAAACTTCCAGCCCATGAGTAGACAGGTGGTTGATGAGATTAACTACAAAGACTATAA AGCTGTCGCCGTACCCTACCAGCATAATAACTCTGGCTTTGTGGGTTACATGGCTCCTACCATGCGTCAGGGTCAAG CGTACCCTGCTAACTACCCATACCCCCTAATTGGAACCACTGCAGTAACCAGTGTCACCCAGAAAAAATTCCTGTGC GACAGGACCATGTGGCGCATCCCATTCTCTAGCAACTTCATGTCCATGGGTGCCCTTACAGACCTGGGACAGAACTT GCTGTATGCCAACTCGGCCCATGCGCTGGACATGACTTTTGAGGTGGATCCCATGGATGAGCCCACCCTGCTTTATC TTCTTTTCGAAGTCTTCGACGTGGTCAGAGTGCACCAGCCACACCGCGGCGTCATCGAGGCCGTCTACCTGCGCACA CCGTTCTCGGCCGGCAACGCCACCACATAAGAAGCCTCTTGCTTCTTGCAAGCAGCAGCTGCAGCCATGTCATGCGG GTCCGGAAACGGCTCCAGCGAGCAAGAGCTCAAAGCCATCGTCCGAGACCTGGGCTGCGGACCCTATTTCCTGGGAA CCTTTGACAAGCGTTTCCCGGGGTTCATGGCCCCCGACAAGCTCGCCTGCGCCATAGTCAACACTGCCGGACGCGAG ACGGGGGGAGAGCACTGGCTGGCTTTTGGTTGGAACCCGCGCTCCAACACCTGCTACCTTTTTGATCCTTTTGGGTT CTCGGATGAGCGACTCAAACAGATTTACCAGTTTGAGTACGAGGGGCTCCTGCGCCGCAGTGCCCTTGCTACCAAAG ACCGCTGCATCACCCTGGAAAAGTCCACCCAGAGCGTGCAGGGCCCGCGCTCAGCCGCCTGTGGACTTTTTTGCTGT ATGTTCCTTCATGCCTTTGTGCACTGGCCCGACCGCCCCATGAACGGAAACCCCACCATGAAGTTGCTGACTGGGGT GTCAAACAGCATGCTCCAATCACCCCAAGTCCAGCCCACCCTGCGCCGCAACCAGGAGGCGCTATATCGCTTCCTAA ACACCCACTCATCTTACTTTCGTTCTCACCGCGCACGCATTGAAAGGGCCACCGCGTTTGACCGTATGGATATGCAA TAAGTCATGTAAAACCGTGTTCAATAAAAAGCATTTTATTTTTACATGCACTAAGGCTCTGGTTTTTTGCTCATTCG TTTTCATCATTCACTCAGAAATCAAATGGGTTCTGGCGGGAGTCAGAGTGACCCGTGGGCAGGGAGACGTTGCGGAA CTGTAACCTGTTCTGCCACTTGAACTCGGGGATCACCAGCTTGGGAACTGGAATTTCGGGAAAGGTGTCTTGCCACA ACTTTCTGGTCAGTTGCAGGGCGCCAAGCAGGTCAGGAGCAGAGATCTTGAAATCACAGTTGGGGCCGGCATTCTGG ACACGGGAGTTGCGGTACACTGGGTTGCAACACTGGAACACCATCAAGGCTGGGTGTCTCACGCTTGCCAGCACGGT CGGGTCACTGATGGTAGTCACATCCAAGTCTTCAGCATTGGCCATTCCAAAGGGGGTCATCTTACAGGTCTGCCTGC CCATCACGGGAGCGCAGCCTGGCTTGTGGTTGCAATCGCAATGAATGGGGATCAGCATCATCCTGGCTTGGTCGGGG GTTATCCCTGGGTACACGGCCTTCATGAAGGCTTCGTACTGCTTGAAAGCTTCCTGAGCCTTACTTCCCTCGGTGTA AAACATCCCACAGGACTTGCTGGAAAATTGGTTAGTAGCACAGTTGGCATCATTCACACAGCAGCGGGCATCGTTGT TGGCCAACTGGACCACATTTCTGCCCCAGCGGTTCTGGGTGATCTTGGCTCTGTCTGGGTTCTCCTTCATAGCGCGC TGCCCGTTTTCGCTCGCCACATCCATCTCGATAATGTGGTCCTTCTGGATCATGATAGTGCCATGCAGGCATTTCAC CTTGCCTTCGTAATCGGTGCATCCATGAGCCCACAGAGCGCACCCGGTGCACTCCCAATTATTGTGGGCGATCTCAG AATAAGAATGCACCAATCCCTGCATGAATCTTCCCATCATCGCTGTCAGGGTCTTCATGCTACTAAATGTCAGCGGG ATGCCACGGTGCTCCTCGTTCACATACTGGTGGCAGATACGCTTGTACTGCTCGTGCTGCTCTGGCATCAGCTTGAA AGAGGTTCTCAGGTCATTATCCAGCCTATACCTCTCCATTAGCACAGCCATCACTTCCATGCCCTTCTCCCAGGCAG ATACCAGGGGCAAGCTCAAAGGATTCCTAACAGCAATAGAAGTAGCTCCTTTAGCTATAGGGTCATTCTTGTCGATC TTCTCAACACTTCTCTTGCCATCCTTCTCAATGATGCGCACCGGGGGGTAGCTGAAGCCCACGGCCACCAACTGAGC CTGTTCTCTTTCTTCTTCGCTGTCCTGGCTGATGTCTTGCAGAGGGACATGCTTGGTCTTCCTGGGCTTCTTCTTGG GAGGGATCGGGGGAGGACTGTTGCTCCGTTCCGGAGACAGGGATGACCGCGAAGTTTCGCTTACCAGTACCACCTGG CTCTCGATAGAAGAATCGGACCCCACGCGACGGTAGGTGTTCCTCTTCGGGGGCAGAGGTGGAGGCGACTGAGATGG GCTGCGGTCCGGCCTTGGAGGCGGATGGCTGGCAGAGCCCATTCCGCGTTCGGGGGTGTGCTCCCGTTGGCGGTCGC TTGACTGATTTCCTCCGCGGCTGGCCATTGTGTTCTCCTAGGCAGAGAAACAACAGACATGGAAACTCAGCCATCAC TGCCAACATCGCTGCAAGCGCCATCACACCTCGCCCCCAGCAGCGACGAGGAGGAGAGCTTAACCACCCCACCACCC AGTCCCGCTACCACCACCTCTACCCTCGATGATGAGGAGGAGGTCGACGCAGCCCAGGAGATGCAGGCGCAGGATAA TGTGAAAGCGGAAGAGATTGAGGCAGATGTCGAGCAGGACCCGGGCTATGTGACACCGGCGGAGCACGAGGAGGAGC TGAAACGTTTTCTAGACAGAGAGGATGACGACCGCCCAGAGCATCACCAGGAGGCTGGCCTCGGGGATCATGTTGCC GACTACCTCTCCGGGCTTGGGAGGGAGGACGTGCTCCTCAAACATCTAGCAAGGCAGTCGATCATAGTTAAAGACGC ACTACTCAACCTCACCGAAGTGCCTATCAGTGTGGAAGAGCTTAGCCGCGCCTACGAGCTGAACCTCTTTTCGCCTC AGATACCCCCCAAGCGGCAGCGAAACGGCACCTGCGAGGCCAACCCTCGACTCAACTTCTATCCAGCTTTTACTGTC CCCGAAGTGCTGGCCACCTACCACATCTTTTTTAAGAACCAAAAGATTCCAGTCTCCTGCCGCGCCAACCGCACCCG CGCAGATGCCCTTCTCAACTTGGGTCCGGGAGCTCGCTTACCTGATATAGCTTCCTTGGAAGAGGTTCCAAAGATCT TTGAGGGTCTGGGAAGTGATGAGACTCGGGCCGCAAATGCTCTGCAACAGGGAGAGAATGGTATGGATGAACATCAC AGCGCTCTAGTGGAACTGGAGGGTGACAATGCCCGGCTTGCAGTGCTCAAGCGCAGTATCGTGGTCACCCATTTTGC CTACCCCGCTGTTAACCTGCCGCCCAAAGTCATGAGCGCTGTCATGGACCATCTGCTCATCAAACGAGCAAGTCCAC TTTCAGAAAACCAGAACATGCAGGATCCAGACGCCTCGGAGGAGGGCAAGCCGGTAGTCAGTGACGAGCAGCTATCT CGCTGGCTGGGTACCAACTCCCCCCGAGATTTGGAAGAAAGACGCAAGCTTATGATGGCTGTAGTGCTAGTAACTGT TGAGTTGGAGTGTCTGCGCCGCTTTTTTACCGACCCCGAGACCCTGCGCAAGCTAGAGGAGAACCTGCACTACACCT TCAGACATGGCTTCGTGCGCCAGGCATGCAAGATCTCCAACGTGGAGCTCACCAACCTGGTTTCATACATGGGCATT TTGCATGAGAACCGGCTAGGGCAGAGCGTTCTGCACACCACCCTGAAGGGGGAGGCCCGCCGCGACTACATCCGAGA CTGTGTCTACCTCTACCTCTGCCATACCTGGCAGACTGGTATGGGTGTGTGGCAACAGTGTTTGGAAGAGCAGAACC TTAAAGAGCTGGACAAGCTCTTGCAGAGATCCCTCAAAGCCCTGTGGACAGGTTTTGACGAGCGCACCGTCGCCTCG GACCTGGCGGACATCATCTTCCCCGAGCGTCTTAGGGTTACTCTGCGAAACGGCCTGCCAGACTTCATGAGCCAGAG CATGCTTAACAACTTTCGCTCTTTCATCCTGGAACGCTCCGGTATCCTGCCTGCCACCTGCTGTGCGCTGCCCTCCG ACTTTGTGCCTCTCACCTACCGCGAGTGCCCACCGCCGCTATGGAGCCACTGCTACCTATTCCGCCTGGCCAACTAC CTCTCCTACCACTCGGATGTGATAGAGGATGTGAGCGGAGACGGCCTGCTGGAATGCCACTGCCGATGCAATTTATG CACACCCCACCGCTCCCTCGCCTGCAACCCCCAGTTGCTAAGCGAGACCCAGATCATCGGCACCTTCGAGTTGCAGG GTCCCAACAGTGAAGGCGAGGGGTCTTCTCCGGGGCAGAGTCTGAAACTGACACCGGGGCTGTGGACCTCCGCCTAC CTGCGCAAGTTTCATCCCGAGGACTATCATCCCTATGAGATCAGGTTCTATGAGGACCAGTCACATCCTCCCAAAGT CGAGCTCTCAGCCTGCGTCATCACCCAGGGGGCAATTCTGGCCCAATTGCAAGCCATCCAAAAATCCCGCCAAGAAT TTCTGCTGAAAAAGGGAAGCGGGGTCTACCTTGACCCCCAGACCGGTGAGGAGCTCAACACAAGGTTCCCCCAGGAT GTCCCATCGCCGAGGAAGCAAGAAGCTGAAGGTGCAGCTGTCACCCCCAGAGGATATGGAGGAAGACTGGGACAGTC AGGCAGAGGAGGAGATGGAAGATTGGGACAGCCAGGCAGAGGAGGTGGACAGCCTGGAGGAAGACAGTTTGGAGGAG GAAGACGAGGAGGCAGAGGAGGTGGAAGAAGCAACCGCCGCCAAACAGTTGTCATCGGCGGCGGAGACAAGCAAGTC CCCAGACAGCAGCACGGCTACCATCTCCGCTCCGGGTCGGGGGGCCCAGCGGCGGCCCAACAGTAGATGGGACGAGA CCGGGCGATTTCCAAACCCGACCACCGCTTCCAAGACCGGTAAGAAGGAGCGACAGGGATACAAGTCCTGGCGTGGA CATAAAAACGCTATCATCTCCTGCTTGCATGAATGCGGGGGCAACATATCCTTCACCCGGCGATACCTGCTTTTCCA CCACGGTGTGAACTTCCCCCGCAATATCTTGCATTACTACCGTCACCTCCACAGCCCCTACTGCAGTCAGCAAGTCC CGGCAACCCCGACAGAAAAAGACAGCAGCGACAACGGTGACCAGAAAAGCAGCAGTTAGAAAATCCACAACAAGTGC AGCAGGAGGAGGACTGAGGATCACAGCGAACGAGCCAGCGCAGACCAGAGAGCTGAGGAACCGGATCTTTCCAACCC TCTATGCCATCTTCCAGCAGAGTCGGGGGCAAGAGCAGGAATTGAAAGTAAAAAACCGATCTCTGCGCTCGCTCACC AGAAGTTGTTTGTATCACAAGAGCGAAGACCAACTTCAGCGCACTCTCGAGGACGCCGAGGCTCTCTTCAACAAGTA CTGCGCGCTGACTCTTAAAGAGTAGCCCTTGCCCGCGCTCATTCGAAAACGGCGGGAATCACGTCACCCTTGGCAGC TGTCCTTTGCCCTCGTCATGAGTAAAGAGATTCCCACGCCTTACATGTGGAGCTATCAGCCCCAAATGGGGTTGGCA GCAGGTGCTTCCCAGGACTACTCCACCCGCATGAATTGGCTTAGCGCCGGGCCCTCAATGATATCACGGGTTAATGA TATACGAGCTTATCGAAACCAGTTACTCCTAGAACAGTCAGCTCTTACCACCACACCCCGCCAACACCTTAATCCCC GAAATTGGCCCGCCGCCCTGGTGTACCAGGAAAATCCCGCTCCCACCACCGTACTACTTCCTCGAGACGCCCAGGCC GAAGTTCAGATGACTAACGCAGGTGTACAGCTGGCGGGCGGTTCCGCCCTATGTCGTCACCGGCCTCAACAGAGTAT AAAACGCCTGGTGATCAGAGGCCGAGGTATCCAGCTCAACGACGAGTCGGTTAGCTCTTCGCTTGGTCTGCGACCAG ACGGAGTCTTCCAGATCGCCGGCTGTGGGAGATCTTCCTTCACTCCTCGTCAGGCTGTGCTGACTTTGGAGAGTTCG TCCTCGCAGCCCCGCTCGGGCGGCATCGGAACTCTCCAGTTTGTGGAGGAGTTTACTCCCTCTGTCTACTTCAACCC CTTCTCCGGCTCTCCTGGCCAGTACCCGGACGAGTTCATACCGAACTTCGACGCAATCAGCGAGTCAGTGGATGGCT ATGATTGATGTCTAATGGTGGCGCGGCTGAGCTAGCTCGACTGCGACACCTAGACCACTGCCGCCGCTTTCGCTGTT TCGCCCGGGAACTCACCGAGTTCATCTACTTCGAACTCTCCGAGGAGCACCCTCAGGGTCCGGCCCACGGAGTGCGG ATTACCATCGAAGGGGGAATAGACTCTCGCCTGCATCGCATCTTCTCCCAGCGGCCCGTGCTGATTGAGCGCGACCA GGGAAATACAACCATCTCCATCTACTGCATCTGTAACCACCCCGGATTGCATGAAAGCCTTTGCTGTCTTGTTTGTG CTGAGTTTAATAAAAACTGAGTTAAGACCTTCCTACGGACTACCGCTTCTTCAATCAGGACTTTACAACACCAACCA GATCTTCCAGAAGACCCAGACCCTTCCTCCTCTGATCCAGGACTCTAACTCTACCTTACCAGCACCCTCCACTACTA ACCTTCCCGAAACTAACAAGCTTGGATCTCATCTGCAACACCGCCTTTCACGAAGCCTTCTTTCTGCCAATACTACC ACTCCCAAAACCGGAGGTGAGCTCCGCGGTCTTCCTACTGACGACCCCTGGGTGGTAGCGGGTTTTGTAACGTTAGG ATTAGTTGCGGGTGGGCTTGTGCTAATCCTTTGCTACCTATACACACCTTGCTGTGCATATTTAGTCATATTGTGCT GTTGGTTTAAGAAATGGGGGCCATACTAGTCGTGCTTGCTTTACTTTCGCTTTTGGGTCTGGGCTCTGCTAATCTCA ATCCTCTTGATCACAATCCATGTCTAGACTTCGACCCAGAAAATTGCACACTTACTTTTGCACCCGACACAAGCCGT CTCTGTGGAGTTCTTATTAAGTGCGGATGGGACTGCAGGTCCGTTGAAATTACACATAATAACAAAACATGGAACAA TACCTTATCCACCACATGGGAGCCAGGAGTTCCCGAGTGGTATACTGTCTCTGTCCGAGGTCCTGACGGTTCCATTC GCATTAGTAACAACACTTTCATTTTTTCTGAAATGTGCGATCTGGCCATGTTTATGAGCAAACAGTATGACCTATGG CCTCCTAGCAAAGAGAACATTGTGGCATTTTCCATTGCTTATTGCTTGGTAACATGCATCATCACTGCTATCATTTG TGTGTGCATACACTTGCTTATAGTTATTCGCCCTAGACAAAGCAATGAGGAAAAAGAGAAAATGCCTTAACCTTTTT CCTCATACCTTTTCTTTACAGCATGGCTTCTGTTACAGCTCTAATTATTGCCAGCATTGTCACTGTCGCTCACGGGC AAACAATTGTCCATATTACCTTAGGACATAATCACACTCTTGTAGGGCCCCCAATTACTTCAGAGGTTATTTGGACC AAACTTGGAAGTGTTGATTATTTTGATATAATTTGCAACAAAACTGAACCAATATTTGTAATCTGTAACAGACAAAA TCTCACGTTAATTAATGTTAGCAAAATTTATAACGGTTACTATTATGGTTATGATAGATCCAGTAGCCAATATAAAA ATTACTTAGTTCGCATAACTCAGCCCAAATCAACAGTGCCAACTATGACAATAATTAAAATGGCTAATAAAGCATTA GAAAATTTTACATTACCAACAACGCCCAATGAAAAAAACATTCCAAATTCAATGATTGCAATTATTGCGGCGGTGGC ATTGGGAATGGCACTAATAATAATATGCATGTTCCTATATGCTTGTTGCTATAAAAAGTTTCAACATAAACAGGATC CACTACTAAATTTTAACATTTAATTTTTTATACAGATGATTTCCACTACAATTTTTATCATTACTAGCCTTGCAGCT GTAACTTATGGCCGTTCACACCTAACTGTACCTGTTGGCTCAACATGTACACTACAAGGACCCCAAGAAGGCTATGT CACTTGGTGGAGAATATATGATAATGGAGGGTTCGCTAGACCATGTGATCAGCCTGGTACAAAATTTTCATGCAACG GAAGAGACTTGACCATTATTAACATAACATTAAATGAGCAAGGCTTCTATTATGGAACCAACTATAAAAATAGTTTA GATTACAACATTATTGTAGTGCCAGCCACCACTTCTGCTCCCCGCAAATCCACTTTCTCTAGCAGCAGTGCCAAAGC AAGCACAATTCCTAAAACAGCTTCTGCTATGTTAAAGCTTCGAAAAATCGCTTTAAGTAATTCCACAGCAGCTCCCA ATACAATTCCTAAATCAACAATTGGCATCATTACTGCCGTGGTAGTGGGATTAATGATTATATTTTTGTGCATAATG TACTACGCCTGCTGCTATAGAAAACATGAACAAAAAGGTGATGCATTACTAAATTTTGATATTTAATTTTTTATAGA ATTATGATATTGTTTCAATCCAATGCCACTAACACTATCAATGTGCAGACTACTTTAAAACATGACATGGAAAACCA CACTACCTCCTATGCATACACAAATATTCAGCCTAAATACGCTATGCAACTAGAAATCACCATACTAATTGTAATTG GAATTCTTATACTATCTGTTATTCTTTATTTTATATTCTGCCGTCAAATACCCAATGTTCATAGAAATTCTAAAAGA CGTCCCATCTATTCTCCTATGATTAGTCGTCCCCATATGGCTCTGAATGAAATCTAAGATCTTTTTTTTTCTTTTAC AGTATGGTGAACATCAATCATGATTCCTAGAAATTTCTTCTTCACCATACTCATCTGTGCTTTCAATGTCTGTGCTA CTTTCACAGCAGTAGCCACTGCAAGCCCAGACTGTATAGGACCATTTGCTTCCTATGCACTTTTTGCCTTTGTTACT TGCATCTGCGTGTGTAGCATAGTCTGCCTGGTTATTAATTTTTTCCAACTGGTAGACTGGATCTTTGTGCGAATTGC CTACCTACGTCACCATCCCGAATACCGCAATCAAAATGTTGCGGCACTTCTTAGGCTTATTTAAAACCATGCAGGCT ATGCTACCAGTTATTTTAATTCTGCTACTACCCTGCATTGCCCTACCTTCCACCGCCACTCGCGCTACACCTGAACA ACTTAGAAAATGCAAATTTCAACAACCATGGTCATTTCTTGATTGCTACCATGAAAAATCTGATTTTCCCACATACT GGATAGTGATTGTTGGAATAATTAACATACTTTCATGTACCTTTTTCTCAATCACAATATACCCCACATTTAATTTT GGGTGGAATTCTCCCAATGCACTGGGTTACCCACAAGAGCTAGATGAACATATCCCACTACAACACATACAACAACC ACTAGCATTGGTAGAGTATGAAAATGAGCCACAACCTTCACTGCCTCCTGCTATTAGTTACTTCAACCTAACCGGCG GAGATGACTGAAATACTCACCACCTCCAATTCCGCCGAGGATCTGCTTGATATGGACGGCCGCGCCTCAGAACAGCG ACTCGCCCAACTACGCATCCGCCAGCAGCAGGAACGCGTGACCAAAGAGCTCAGAGATGTCATCCAAATTCACCAAT GCAAAAAAGGCATATTTTGTTTGGTAAAACAAGCCAAGATATCCTACGAGATCACCGCTACTGACCATCGCCTCTCT TACGAACTTGGCCCCCAACGACAAAAATTTACATGCATGGTGGGAATCAACCCTATAGTTATCACCCAGCAAAGTGG AGATACTAAGGGTTGCATTCACTGCTCTTGCGATTCCACCGAGTGCACCTACACCCTGCTGAAGACCCTATGCGGCC TAAGAGACCTGCTACCCATGAATTAAAAATTAATAAAAAATTACTTACTTGAAATCAGCAATAAGGTCTCTGTTGAA ATTTTTTCCCAGCAGCACCTCGCTTCCCTCTTCCCAACTCTGGTATTCTAAACCCCGTTCAGCGGCATACTTTCTCC ATACTTTAAAGGGGATGTCAAATTTTAGCTCCTCTCCTGTACCCACGATCTTCATGTCTTTCTTCCCAGATGACCAA GAGAGTCCGGCTCAGTGATTCCTTCAACCCTGTCTACCCCTATGAAGACGAAAGCACCTCCCAACACCCCTTTATAA ACCCAGGGTTTATTTCCCCAAATGGCTTTACACAAAGCCCAGACGGAGTTCTTACTTTAAATTGTTTAACCCCACTA ACAACCACAGGCGGGCCTTTACAGTTAAAAGTGGGAGGGGGACTTATAGTGGATGACACTGATGGGACCTTACAAGA AAACATACGTGTTACAGCACCCATTACTAAAAATAATCATTCTGTAGAACTATCCATTGGAAATGGATTAGAAACAC AAAACAATAAACTATGTGCCAAATTGGGAAATGGGTTAAAATTTAACAACGGTGACATTTGTATAAAGGATAGTATT AACACCTTATGGACTGGAATAAAGCCTCCACCTAACTGTCAAATAGTGGAAAACACTGATACAAACGATGGCAAACT TACTTTAGTATTAGTAAAAAACGGAGGACTTGTTAATGGCTACGTATCTCTAGTTGGTGTATCAGACACTGTGAACC AAATGTTCACACAAAAGTCAGCAACCATACAATTAAGATTATATTTCGACTCTTCTGGAAATCTATTAACTGATGAA TCAAACTTAAAAATTCCACTTAAAAATAAATCTTCTACAGCAACCAGTGAAGCTGCAACCAGCAGCAAAGCCTTTAT GCCAAGTACTACAGCTTATCCCTTTAACACCACTACTAGGGATAGTGAAAACTATATTCATGGAATATGTTACTATA TGACTAGTTATGATAGAAGTCTAGTTCCCTTAAACATTTCTATAATGCTAAACAGCCGTACGATTTCTTCCAATGTT GCCTATGCCATACAATTTGAATGGAATCTAAATGCAAAAGAATCTCCAGAAAGCAACATAGCTACGCTGACCACATC CCCCTTTTTCTTTTCTTATATTAGAGAAGACGACAACTAAAAAATAAAGTTTAAGTGTTTTTATTTAAAAATCACAA AATTCGAGTAGTTATTTTGCCTCCCCCTTCCCATTTAACAGAATACACCAATCTCTCCCCACGCACAGCTTTAAACA TTTGGATACCATTAGAGATAGACATAGTTTTAGTTTCCACATTCCAAACAGTTTCAGAGCGAGCCAATCTGGGGTCA GTGATACATAAAAATGCATCGGGATAGTCTTTTAAAGCGCTTTCACAGTCCAACTGCTGCGGATGCGACTCCGGAGT CTGGATCACAGTCATCTGGAAGAAGAACGATGGGAATCATAATCCGAAAACGGAATCGGGCGATTGTGTCTCATCAA ACCCACAAGCAGCCGCTGTCTGCGTCGCTCCGTGCGACTGCTGTTTATGGGATCGGGGTCTGCAGTGTCCTGAAGCA TGATTTTAATAGCCCTTAACATTAACTTTCTGGTGCGATGCGCGCAGCAACGCATTCTGATTTCACTGAGATTACTA CAGTATGTACAGCACATTATCACAATATTGTTTAATAAACCATAATTAAAAGCGCTCCAGCCAAAACTCATATCTGA TACAATCGCCCCTGCATGACCATCATACCAAATTTTAATATAAATTAAATGTCGTTCCCTCAAAAACACACTACCCA CATACATGATCTCTTTTGGCATGTGCATATTAACAATCTGTCTGTACCATGGACAACGTTGGTTAATCATGCAACCC AATATAACCTTCCGGAACCACACTGCCAACACCGCTCCCCCAGCCATGCATTGAAGTGAACCCTGCTGATTACAATG ACAATGAAGAACCCAATTCTCTCGACCATGAATCACTTGAGACTGAAAAATATCTATAGTAGCACAACAAAGACATA AATGCATGCATCTTCTCATAATTTTTAACTCATCTGGATTTAAAAACATATCCCAAGGAATGGGAAACTCTTGCAAA ACAGTAAAGCTGGCAGAACAAGGAAGACCACGAACACAACTTACACTATGCATAGTCATAGTATCACAATCTGGCAA CAGCGGGTGGTCTTCAGTCATAGAAGCTCGGGTTTCATTTTCCTCACATCGTGGTAACTGGGCTCTGGTGTAAGGGT GATGTCTGGCGCATGATGTCGAGCGTGCGCGCAACCTTGTCATAATGGAGTTGTTTCCTGACATTCTCGTATTTTGT ATAGCAAAACGCGGCCCTGGCACAACACACTCTTCTTCGTCTTCTATCCTGCCGCTTAGTGTGTTCCGTCTGATAAT TCAAGTACAGCCACACTCTTAAGTTGGTCAAAAGAATGCTGGCTTCAGTTGTAATCAAAACTCCATCATATTTAATT GTTCTAAGGAAATCATCCACGGTAGCATATGCAAATCCCAACCAAGCAATGCAACTGGATTGCGTTTCAAGCAGCAG AGGAGAGGGAAGAGACGGAAGAATCATGTTAATTTTTATTCCAAACGATCTCGCAGTACTTCAAATTGTAGATCGCG CAGATGGCATCTATCGCCCCCACTGTGTTGGTGAAAAAGCACAGCTAAATCAAAAGAAATGCGATTTTCAAGGTGCT CAACGGTGGCTTCCAACAAAGCCTCCACGCGCACATCCAAAAACAAAAGAATACCAAAAGAAGGAGCATTTTCTAAC TCCTCAAACATCATATTACATTCCTGCACCATTCCCAGATAATTTTCAGCTTTCCAGCCTTGAATTATTCGTGTCAG TTCTTGTGGTAAATCCAAACCACACATTACAAACAGGTCCCGGAGGGCGCCCTCCACCACCATTCTTAAACACACCC TCATAATGACAAAATATCTTGCTCCTGTGTCACCTGTAGCAAATTAAGAATGGCATCATCAATTGACATGCCCTTGG CTCTAAGTTCTTCTCTAAGTTCTAGTTGTAAATACTCTCTCATATTATCACCAAACTGCTTAGCCAAAAGCCCCCCG GGAACAATAGCAGGGGACGCTACAGTGCAGTACAAGCGCAGACCTCCCCAATTGGCTCCAGCAAAAACAAGATTAGA ATAAGCATACTGGGAACCACCAGTAATATCATCAAAGTTGCTGGAAATATAATCAGGCAGAATTTCTTGTAAAAATT GAATAAAAGAAAAATTTTCCAAAGAAACATTCAAAATCTCTGGGATGCAAATGCAATAGGTTACCGCGCTGCGCTCC AACATTGTTAGTTTTGAATTAGTCTGCAAAATAAAAGAAACAAGCGTCATATCATAGTAGCCTGTCGAACAGGTGGA TAAATCAGTCTTTCCATCACAAGACAAGCCACAGGGTCTCCAGCTCGACCCTCGTAAAACCTGTCATCGTGATTAAA CAACAGCACCGAAAGTTCCTCGCGGTGGCCAGCATGAATAATTCTTGATGAAGCATATAATCCAGACATGTTAGCAT CAGTTAAAGAGAAAAAACAGCCAACATAGCCTCTGGGTATAATTATGCTTAATCTTAAGTATAGCAAAGCCACCCCT CGCGGATACAAAGTAAAAGGCACAGGAGAATAAAAAATATAATTATTTCTCTGCTGCTGTTCAGGCAACGTCGCCCC CGGTCCATCTAAATACACATACAAAGCCTCATCAGCCATGGCTTACCAGACAAAGTACAGCGGGCGCACAAAGCACA AGCTCTAAAGAAGCTCTAAAGACACTCTTCAACCTCTCCACAATATATACACAAGCCCTAAACTGACGTAATGGGAG TAAAGTGTAAAAAATCCCGCCAAGCCCAACACACACCCCGAAACTGCGTCAGCAGGGAAAAGTACAGTTTCACTTCC GCATTCCCAACAAGCGTAAGTTCCTCTTTCTCATGGTACGTCACATCCGATTAACTTGCAACGTCATTTTCCCACGG TCGCACCGCCCCTTTTAGCCGTTCACCCCGCAGCCAATCACCACACAGCGCGCACTTTTTTAAATTACCTCATTTAC ATATTGGCACCATTCCATCTATAAGGTATATTATATTGATTG

GenBank Accession No. AAW33547 MTKRVRLSDSFNPVYPYEDESTSQHPFINPGFISPNGFTQSPDGVLTLNCLTPLTTTGGPLQLKVGGGLIVDDTDGT LQENIRVTAPITKNNHSVELSIGNGLETONNKLCAKLGNGLKFNNGDICIKDSINTLWTGIKPPPNCQIVENTDTND GKLTLVLVKNGGLVNGYVSLVGVSDTVNQMFTQKSATIQLRLYFDSSGNLLTDESNLKIPLKNKSSTATSEAATSSK AFMPSTTAYPENTTTRDSENYIHGICYYMTSYDRSLVPLNISIMLNSRTISSNVAYAIQFEWNLNAKESPESNIATL TTSPFFFSYIREDDN

GenBank Accession No. AAW33525 MRRTVLGGAVVYPEGPPPSYESVMQQAAAAAMQPPLEAPFVPPRYLAPTEGRNSIRYSELAPLYDTTRLYLVDNKSA DIASLNYQNDHSNFLTTVVONNDFTPTEASTQTINFDERSRWGGQLKTIMHTNMPNVNEYMFSNKFKARVMVSRKAP EGVTVDDNYDHKQDILEYEWFEFTLPEGNFSATMTIDLMNNAIIDNYLEVGRONGVLESDIGVKFDTRNFRLGWDPE TKLIMPGVYTYEAFHPDIVLLPGCGVDFTESRLSNLLGIRKRHPFQEGFKILYEDLEGGNIPALLDVEAYKNSKKER EAKTEAAKAAAIAKANIVVSDPVRVANAEEVRGDNYTASSVATEESLLAAVAETTETKLTIKPVEKDSKSRSYNVLE DKVNTAYRSWYLSYNYGDPEKGVRSWTLLTTSDVTCGAEQVYWSLPDMMQDPVTFRSTRQVSNYPVVGAELMPVFSK SFYNEQAVYSQQLRQSTSLTHVENREPENQILIRPPAPTITTVSENVPALTDHGTLPLRSSIRGVQRVTVTDARRRT CPYVYKALGIVAPRVLSSRTF

GenBank Accession No. AAW33530 MATPSMLPQWAYMHIAGQDASEYLSPGLVQFARATDTYENLGNKERNPTVAPTHDVTTDRSQRLMLRFVPVDREDNT YAYKVRYTLAVGDNRVLDMASTFFDIRGVLDRGPSFKPYSGTAYNSLAPKGAPNTSQWLNKGDEEDGEDDQQATYTF GNAPVKAEAEITKEGLPIGLEVPSEGGPKPIYADKLYQPEPQVGEESWTDTDGTDEKYGGRALKPETKMKPCYGSFA KPTNVKGGQAKVKKEEEGKVEYDIDMNFFDLRSQMTGLKPKIVMYAENVDLETPDTHVVYKPGASDASSHANLGQQS MPNRPNYIGERDNFIGLMYYNSTGNMGVLAGQASQLNAVVDLQDRNTELSYQLLLDSLGDRTRYFSMWNQAVDSYDP DVRVIENHGVEDELPNYCFPLDGVGPRIDSYKGIETNGDETTTWKDLEPKGISEIAKGNPFAMEINLQANLWRSFLY SNVALYLPDSYKYTPANVTLPTNTNTYDYMNGRVVPPSLVDTYVNIGARWSLDAMDNVNPFNHHRNAGLRYRSMLLG NGRYVPFHIQVPQKFFAVKNLLLLPGSYTYEWNFRKDVNMVLQSSLGNDLRVDGASISFTSINLYATFFPMAHNTAS TLEAMLRNDTNDQSENDYLSAANMLYPIPANATNVPISIPSRNWAAFRGWSFTRLKTKETPSLGSGEDPYFVYSGSI PYLDGTFYLNHTFKKVSIMFDSSVSWPGNDRLLSPNEFEIKRTVDGEGYNVAQCNMTKDWFLVOMLANYNIGYQGFY VPEGYKDRMYSFFRNFQPMSROVVDEINYKDYKAVAVPYQHNNSGFVGYMAPTMROGQAYPANYPYPLIGTTAVTSV TOKKFLCDRTMWRIPFSSNFMSMGALTDLGQNLLYANSAHALDMTFEVDPMDEPTLLYLLFEVEDVVRVHOPHRGVI EAVYLRTPFSAGNATT

GenBank Accession No. AC 000008 (SEQ ID NO: 209) CATCATCAATAATATACCTTATTTTGGATTGAAGCCAATATGATAATGAGGGGGTGGAGTTTGTGACGTGGCGCGGG GCGTGGGAACGGGGGGGGTGACGTAGTAGTGTGGCGGAAGTGTGATGTTGCAAGTGTGGCGGAACACATGTAAGCGA CGGATGTGGCAAAAGTGACGTTTTTGGTGTGCGCCGGTGTACACAGGAAGTGACAATTTTCGCGCGGTTTTAGGCGG ATGTTGTAGTAAATTTGGGCGTAACCGAGTAAGATTTGGCCATTTTCGCGGGAAAACTGAATAAGAGGAAGTGAAAT CTGAATAATTTTGTGTTACTCATAGCGCGTAATATTTGTCTAGGGCCGCGGGGACTTTGACCGTTTACGTGGAGACT CGCCCAGGTGTTTTTCTCAGGTGTTTTCCGCGTTCCGGGTCAAAGTTGGCGTTTTATTATTATAGTCAGCTGACGTG TAGTGTATTTATACCCGGTGAGTTCCTCAAGAGGCCACTCTTGAGTGCCAGCGAGTAGAGTTTTCTCCTCCGAGCCG CTCCGACACCGGGACTGAAAATGAGACATATTATCTGCCACGGAGGTGTTATTACCGAAGAAATGGCCGCCAGTCTT TTGGACCAGCTGATCGAAGAGGTACTGGCTGATAATCTTCCACCTCCTAGCCATTTTGAACCACCTACCCTTCACGA ACTGTATGATTTAGACGTGACGGCCCCCGAAGATCCCAACGAGGAGGCGGTTTCGCAGATTTTTCCCGACTCTGTAA TGTTGGCGGTGCAGGAAGGGATTGACTTACTCACTTTTCCGCCGGCGCCCGGTTCTCCGGAGCCGCCTCACCTTTCC CGGCAGCCCGAGCAGCCGGAGCAGAGAGCCTTGGGTCCGGTTTCTATGCCAAACCTTGTACCGGAGGTGATCGATCT TACCTGCCACGAGGCTGGCTTTCCACCCAGTGACGACGAGGATGAAGAGGGTGAGGAGTTTGTGTTAGATTATGTGG AGCACCCCGGGCACGGTTGCAGGTCTTGTCATTATCACCGGAGGAATACGGGGGACCCAGATATTATGTGTTCGCTT TGCTATATGAGGACCTGTGGCATGTTTGTCTACAGTAAGTGAAAATTATGGGCAGTGGGTGATAGAGTGGTGGGTTT GGTGTGGTAATTTTTTTTTTAATTTTTACAGTTTTGTGGTTTAAAGAATTTTGTATTGTGATTTTTTTAAAAGGTCC TGTGTCTGAACCTGAGCCTGAGCCCGAGCCAGAACCGGAGCCTGCAAGACCTACCCGCCGTCCTAAAATGGCGCCTG CTATCCTGAGACGCCCGACATCACCTGTGTCTAGAGAATGCAATAGTAGTACGGATAGCTGTGACTCCGGTCCTTCT AACACACCTCCTGAGATACACCCGGTGGTCCCGCTGTGCCCCATTAAACCAGTTGCCGTGAGAGTTGGTGGGCGTCG CCAGGCTGTGGAATGTATCGAGGACTTGCTTAACGAGCCTGGGCAACCTTTGGACTTGAGCTGTAAACGCCCCAGGC CATAAGGTGTAAACCTGTGATTGCGTGTGTGGTTAACGCCTTTGTTTGCTGAATGAGTTGATGTAAGTTTAATAAAG GGTGAGATAATGTTTAACTTGCATGGCGTGTTAAATGGGGCGGGGCTTAAAGGGTATATAATGCGCCGTGGGCTAAT CTTGGTTACATCTGACCTCATGGAGGCTTGGGAGTGTTTGGAAGATTTTTCTGCTGTGCGTAACTTGCTGGAACAGA GCTCTAACAGTACCTCTTGGTTTTGGAGGTTTCTGTGGGGCTCATCCCAGGCAAAGTTAGTCTGCAGAATTAAGGAG GATTACAAGTGGGAATTTGAAGAGCTTTTGAAATCCTGTGGTGAGCTGTTTGATTCTTTGAATCTGGGTCACCAGGC GCTTTTCCAAGAGAAGGTCATCAAGACTTTGGATTTTTCCACACCGGGGCGCGCTGCGGCTGCTGTTGCTTTTTTGA GTTTTATAAAGGATAAATGGAGCGAAGAAACCCATCTGAGCGGGGGGTACCTGCTGGATTTTCTGGCCATGCATCTG TGGAGAGCGGTTGTGAGACACAAGAATCGCCTGCTACTGTTGTCTTCCGTCCGCCCGGCGATAATACCGACGGAGGA GCAGCAGCAGCAGCAGGAGGAAGCCAGGCGGCGGCGGCAGGAGCAGAGCCCATGGAACCCGAGAGCCGGCCTGGACC CTCGGGAATGAATGTTGTACAGGTGGCTGAACTGTATCCAGAACTGAGACGCATTTTGACAATTACAGAGGATGGGC AGGGGCTAAAGGGGGTAAAGAGGGAGCGGGGGGCTTGTGAGGCTACAGAGGAGGCTAGGAATCTAGCTTTTAGCTTA ATGACCAGACACCGTCCTGAGTGTATTACTTTTCAACAGATCAAGGATAATTGCGCTAATGAGCTTGATCTGCTGGC GCAGAAGTATTCCATAGAGCAGCTGACCACTTACTGGCTGCAGCCAGGGGATGATTTTGAGGAGGCTATTAGGGTAT ATGCAAAGGTGGCACTTAGGCCAGATTGCAAGTACAAGATCAGCAAACTTGTAAATATCAGGAATTGTTGCTACATT TCTGGGAACGGGGCCGAGGTGGAGATAGATACGGAGGATAGGGTGGCCTTTAGATGTAGCATGATAAATATGTGGCC GGGGGTGCTTGGCATGGACGGGGTGGTTATTATGAATGTAAGGTTTACTGGCCCCAATTTTAGCGGTACGGTTTTCC TGGCCAATACCAACCTTATCCTACACGGTGTAAGCTTCTATGGGTTTAACAATACCTGTGTGGAAGCCTGGACCGAT GTAAGGGTTCGGGGCTGTGCCTTTTACTGCTGCTGGAAGGGGGTGGTGTGTCGCCCCAAAAGCAGGGCTTCAATTAA GAAATGCCTCTTTGAAAGGTGTACCTTGGGTATCCTGTCTGAGGGTAACTCCAGGGTGCGCCACAATGTGGCCTCCG ACTGTGGTTGCTTCATGCTAGTGAAAAGCGTGGCTGTGATTAAGCATAACATGGTATGTGGCAACTGCGAGGACAGG GCCTCTCAGATGCTGACCTGCTCGGACGGCAACTGTCACCTGCTGAAGACCATTCACGTAGCCAGCCACTCTCGCAA GGCCTGGCCAGTGTTTGAGCATAACATACTGACCCGCTGTTCCTTGCATTTGGGTAACAGGAGGGGGGTGTTCCTAC CTTACCAATGCAATTTGAGTCACACTAAGATATTGCTTGAGCCCGAGAGCATGTCCAAGGTGAACCTGAACGGGGTG TTTGACATGACCATGAAGATCTGGAAGGTGCTGAGGTACGATGAGACCCGCACCAGGTGCAGACCCTGCGAGTGTGG CGGTAAACATATTAGGAACCAGCCTGTGATGCTGGATGTGACCGAGGAGCTGAGGCCCGATCACTTGGTGCTGGCCT GCACCCGCGCTGAGTTTGGCTCTAGCGATGAAGATACAGATTGAGGTACTGAAATGTGTGGGCGTGGCTTAAGGGTG GGAAAGAATATATAAGGTGGGGGTCTTATGTAGTTTTGTATCTGTTTTGCAGCAGCCGCCGCCGCCATGAGCACCAA CTCGTTTGATGGAAGCATTGTGAGCTCATATTTGACAACGCGCATGCCCCCATGGGCCGGGGTGCGTCAGAATGTGA TGGGCTCCAGCATTGATGGTCGCCCCGTCCTGCCCGCAAACTCTACTACCTTGACCTACGAGACCGTGTCTGGAACG CCGTTGGAGACTGCAGCCTCCGCCGCCGCTTCAGCCGCTGCAGCCACCGCCCGCGGGATTGTGACTGACTTTGCTTT CCTGAGCCCGCTTGCAAGCAGTGCAGCTTCCCGTTCATCCGCCCGCGATGACAAGTTGACGGCTCTTTTGGCACAAT TGGATTCTTTGACCCGGGAACTTAATGTCGTTTCTCAGCAGCTGTTGGATCTGCGCCAGCAGGTTTCTGCCCTGAAG GCTTCCTCCCCTCCCAATGCGGTTTAAAACATAAATAAAAAACCAGACTCTGTTTGGATTTGGATCAAGCAAGTGTC TTGCTGTCTTTATTTAGGGGTTTTGCGCGCGCGGTAGGCCCGGGACCAGCGGTCTCGGTCGTTGAGGGTCCTGTGTA TTTTTTCCAGGACGTGGTAAAGGTGACTCTGGATGTTCAGATACATGGGCATAAGCCCGTCTCTGGGGTGGAGGTAG CACCACTGCAGAGCTTCATGCTGCGGGGTGGTGTTGTAGATGATCCAGTCGTAGCAGGAGCGCTGGGCGTGGTGCCT AAAAATGTCTTTCAGTAGCAAGCTGATTGCCAGGGGCAGGCCCTTGGTGTAAGTGTTTACAAAGCGGTTAAGCTGGG ATGGGTGCATACGTGGGGATATGAGATGCATCTTGGACTGTATTTTTAGGTTGGCTATGTTCCCAGCCATATCCCTC CGGGGATTCATGTTGTGCAGAACCACCAGCACAGTGTATCCGGTGCACTTGGGAAATTTGTCATGTAGCTTAGAAGG AAATGCGTGGAAGAACTTGGAGACGCCCTTGTGACCTCCAAGATTTTCCATGCATTCGTCCATAATGATGGCAATGG GCCCACGGGCGGCGGCCTGGGCGAAGATATTTCTGGGATCACTAACGTCATAGTTGTGTTCCAGGATGAGATCGTCA TAGGCCATTTTTACAAAGCGCGGGCGGAGGGTGCCAGACTGCGGTATAATGGTTCCATCCGGCCCAGGGGCGTAGTT ACCCTCACAGATTTGCATTTCCCACGCTTTGAGTTCAGATGGGGGGATCATGTCTACCTGCGGGGCGATGAAGAAAA CGGTTTCCGGGGTAGGGGAGATCAGCTGGGAAGAAAGCAGGTTCCTGAGCAGCTGCGACTTACCGCAGCCGGTGGGC CCGTAAATCACACCTATTACCGGGTGCAACTGGTAGTTAAGAGAGCTGCAGCTGCCGTCATCCCTGAGCAGGGGGGC CACTTCGTTAAGCATGTCCCTGACTCGCATGTTTTCCCTGACCAAATCCGCCAGAAGGCGCTCGCCGCCCAGCGATA GCAGTTCTTGCAAGGAAGCAAAGTTTTTCAACGGTTTGAGACCGTCCGCCGTAGGCATGCTTTTGAGCGTTTGACCA AGCAGTTCCAGGCGGTCCCACAGCTCGGTCACCTGCTCTACGGCATCTCGATCCAGCATATCTCCTCGTTTCGCGGG TTGGGGCGGCTTTCGCTGTACGGCAGTAGTCGGTGCTCGTCCAGACGGGCCAGGGTCATGTCTTTCCACGGGCGCAG GGTCCTCGTCAGCGTAGTCTGGGTCACGGTGAAGGGGTGCGCTCCGGGCTGCGCGCTGGCCAGGGTGCGCTTGAGGC TGGTCCTGCTGGTGCTGAAGCGCTGCCGGTCTTCGCCCTGCGCGTCGGCCAGGTAGCATTTGACCATGGTGTCATAG TCCAGCCCCTCCGCGGCGTGGCCCTTGGCGCGCAGCTTGCCCTTGGAGGAGGCGCCGCACGAGGGGCAGTGCAGACT TTTGAGGGCGTAGAGCTTGGGCGCGAGAAATACCGATTCCGGGGAGTAGGCATCCGCGCCGCAGGCCCCGCAGACGG TCTCGCATTCCACGAGCCAGGTGAGCTCTGGCCGTTCGGGGTCAAAAACCAGGTTTCCCCCATGCTTTTTGATGCGT TTCTTACCTCTGGTTTCCATGAGCCGGTGTCCACGCTCGGTGACGAAAAGGCTGTCCGTGTCCCCGTATACAGACTT GAGAGGCCTGTCCTCGAGCGGTGTTCCGCGGTCCTCCTCGTATAGAAACTCGGACCACTCTGAGACAAAGGCTCGCG TCCAGGCCAGCACGAAGGAGGCTAAGTGGGAGGGGTAGCGGTCGTTGTCCACTAGGGGGTCCACTCGCTCCAGGGTG TGAAGACACATGTCGCCCTCTTCGGCATCAAGGAAGGTGATTGGTTTGTAGGTGTAGGCCACGTGACCGGGTGTTCC TGAAGGGGGGCTATAAAAGGGGGTGGGGGCGCGTTCGTCCTCACTCTCTTCCGCATCGCTGTCTGCGAGGGCCAGCT GTTGGGGTGAGTACTCCCTCTGAAAAGCGGGCATGACTTCTGCGCTAAGATTGTCAGTTTCCAAAAACGAGGAGGAT TTGATATTCACCTGGCCCGCGGTGATGCCTTTGAGGGTGGCCGCATCCATCTGGTCAGAAAAGACAATCTTTTTGTT GTCAAGCTTGGTGGCAAACGACCCGTAGAGGGCGTTGGACAGCAACTTGGCGATGGAGCGCAGGGTTTGGTTTTTGT CGCGATCGGCGCGCTCCTTGGCCGCGATGTTTAGCTGCACGTATTCGCGCGCAACGCACCGCCATTCGGGAAAGACG GTGGTGCGCTCGTCGGGCACCAGGTGCACGCGCCAACCGCGGTTGTGCAGGGTGACAAGGTCAACGCTGGTGGCTAC CTCTCCGCGTAGGCGCTCGTTGGTCCAGCAGAGGCGGCCGCCCTTGCGCGAGCAGAATGGCGGTAGGGGGTCTAGCT GCGTCTCGTCCGGGGGGTCTGCGTCCACGGTAAAGACCCCGGGCAGCAGGCGCGCGTCGAAGTAGTCTATCTTGCAT CCTTGCAAGTCTAGCGCCTGCTGCCATGCGCGGGCGGCAAGCGCGCGCTCGTATGGGTTGAGTGGGGGACCCCATGG CATGGGGTGGGTGAGCGCGGAGGCGTACATGCCGCAAATGTCGTAAACGTAGAGGGGCTCTCTGAGTATTCCAAGAT ATGTAGGGTAGCATCTTCCACCGCGGATGCTGGCGCGCACGTAATCGTATAGTTCGTGCGAGGGAGCGAGGAGGTCG GGACCGAGGTTGCTACGGGCGGGCTGCTCTGCTCGGAAGACTATCTGCCTGAAGATGGCATGTGAGTTGGATGATAT GGTTGGACGCTGGAAGACGTTGAAGCTGGCGTCTGTGAGACCTACCGCGTCACGCACGAAGGAGGCGTAGGAGTCGC GCAGCTTGTTGACCAGCTCGGCGGTGACCTGCACGTCTAGGGCGCAGTAGTCCAGGGTTTCCTTGATGATGTCATAC TTATCCTGTCCCTTTTTTTTCCACAGCTCGCGGTTGAGGACAAACTCTTCGCGGTCTTTCCAGTACTCTTGGATCGG AAACCCGTCGGCCTCCGAACGGTAAGAGCCTAGCATGTAGAACTGGTTGACGGCCTGGTAGGCGCAGCATCCCTTTT CTACGGGTAGCGCGTATGCCTGCGCGGCCTTCCGGAGCGAGGTGTGGGTGAGCGCAAAGGTGTCCCTGACCATGACT TTGAGGTACTGGTATTTGAAGTCAGTGTCGTCGCATCCGCCCTGCTCCCAGAGCAAAAAGTCCGTGCGCTTTTTGGA ACGCGGATTTGGCAGGGCGAAGGTGACATCGTTGAAGAGTATCTTTCCCGCGCGAGGCATAAAGTTGCGTGTGATGC GGAAGGGTCCCGGCACCTCGGAACGGTTGTTAATTACCTGGGCGGCGAGCACGATCTCGTCAAAGCCGTTGATGTTG TGGCCCACAATGTAAAGTTCCAAGAAGCGCGGGATGCCCTTGATGGAAGGCAATTTTTTAAGTTCCTCGTAGGTGAG CTCTTCAGGGGAGCTGAGCCCGTGCTCTGAAAGGGCCCAGTCTGCAAGATGAGGGTTGGAAGCGACGAATGAGCTCC ACAGGTCACGGGCCATTAGCATTTGCAGGTGGTCGCGAAAGGTCCTAAACTGGCGACCTATGGCCATTTTTTCTGGG GTGATGCAGTAGAAGGTAAGCGGGTCTTGTTCCCAGCGGTCCCATCCAAGGTTCGCGGCTAGGTCTCGCGCGGCAGT CACTAGAGGCTCATCTCCGCCGAACTTCATGACCAGCATGAAGGGCACGAGCTGCTTCCCAAAGGCCCCCATCCAAG TATAGGTCTCTACATCGTAGGTGACAAAGAGACGCTCGGTGCGAGGATGCGAGCCGATCGGGAAGAACTGGATCTCC CGCCACCAATTGGAGGAGTGGCTATTGATGTGGTGAAAGTAGAAGTCCCTGCGACGGGCCGAACACTCGTGCTGGCT TTTGTAAAAACGTGCGCAGTACTGGCAGCGGTGCACGGGCTGTACATCCTGCACGAGGTTGACCTGACGACCGCGCA CAAGGAAGCAGAGTGGGAATTTGAGCCCCTCGCCTGGCGGGTTTGGCTGGTGGTCTTCTACTTCGGCTGCTTGTCCT TGACCGTCTGGCTGCTCGAGGGGAGTTACGGTGGATCGGACCACCACGCCGCGCGAGCCCAAAGTCCAGATGTCCGC GCGCGGCGGTCGGAGCTTGATGACAACATCGCGCAGATGGGAGCTGTCCATGGTCTGGAGCTCCCGCGGCGTCAGGT CAGGCGGGAGCTCCTGCAGGTTTACCTCGCATAGACGGGTCAGGGCGCGGGCTAGATCCAGGTGATACCTAATTTCC AGGGGCTGGTTGGTGGCGGCGTCGATGGCTTGCAAGAGGCCGCATCCCCGCGGCGCGACTACGGTACCGCGCGGCGG GCGGTGGGCCGCGGGGGTGTCCTTGGATGATGCATCTAAAAGCGGTGACGCGGGCGAGCCCCCGGAGGTAGGGGGGG CTCCGGACCCGCCGGGAGAGGGGGCAGGGGCACGTCGGCGCCGCGCGCGGGCAGGAGCTGGTGCTGCGCGCGTAGGT TGCTGGCGAACGCGACGACGCGGCGGTTGATCTCCTGAATCTGGCGCCTCTGCGTGAAGACGACGGGCCCGGTGAGC TTGAGCCTGAAAGAGAGTTCGACAGAATCAATTTCGGTGTCGTTGACGGCGGCCTGGCGCAAAATCTCCTGCACGTC TCCTGAGTTGTCTTGATAGGCGATCTCGGCCATGAACTGCTCGATCTCTTCCTCCTGGAGATCTCCGCGTCCGGCTC GCTCCACGGTGGCGGCGAGGTCGTTGGAAATGCGGGCCATGAGCTGCGAGAAGGCGTTGAGGCCTCCCTCGTTCCAG ACGCGGCTGTAGACCACGCCCCCTTCGGCATCGCGGGCGCGCATGACCACCTGCGCGAGATTGAGCTCCACGTGCCG GGCGAAGACGGCGTAGTTTCGCAGGCGCTGAAAGAGGTAGTTGAGGGTGGTGGCGGTGTGTTCTGCCACGAAGAAGT ACATAACCCAGCGTCGCAACGTGGATTCGTTGATATCCCCCAAGGCCTCAAGGCGCTCCATGGCCTCGTAGAAGTCC ACGGCGAAGTTGAAAAACTGGGAGTTGCGCGCCGACACGGTTAACTCCTCCTCCAGAAGACGGATGAGCTCGGCGAC AGTGTCGCGCACCTCGCGCTCAAAGGCTACAGGGGCCTCTTCTTCTTCTTCAATCTCCTCTTCCATAAGGGCCTCCC CTTCTTCTTCTTCTGGCGGCGGTGGGGGAGGGGGGACACGGCGGCGACGACGGCGCACCGGGAGGCGGTCGACAAAG CGCTCGATCATCTCCCCGCGGCGACGGCGCATGGTCTCGGTGACGGCGCGGCCGTTCTCGCGGGGGCGCAGTTGGAA GACGCCGCCCGTCATGTCCCGGTTATGGGTTGGCGGGGGGCTGCCATGCGGCAGGGATACGGCGCTAACGATGCATC TCAACAATTGTTGTGTAGGTACTCCGCCGCCGAGGGACCTGAGCGAGTCCGCATCGACCGGATCGGAAAACCTCTCG AGAAAGGCGTCTAACCAGTCACAGTCGCAAGGTAGGCTGAGCACCGTGGCGGGCGGCAGCGGGCGGCGGTCGGGGTT GTTTCTGGCGGAGGTGCTGCTGATGATGTAATTAAAGTAGGCGGTCTTGAGACGGCGGATGGTCGACAGAAGCACCA TGTCCTTGGGTCCGGCCTGCTGAATGCGCAGGCGGTCGGCCATGCCCCAGGCTTCGTTTTGACATCGGCGCAGGTCT TTGTAGTAGTCTTGCATGAGCCTTTCTACCGGCACTTCTTCTTCTCCTTCCTCTTGTCCTGCATCTCTTGCATCTAT CGCTGCGGCGGCGGCGGAGTTTGGCCGTAGGTGGCGCCCTCTTCCTCCCATGCGTGTGACCCCGAAGCCCCTCATCG GCTGAAGCAGGGCTAGGTCGGCGACAACGCGCTCGGCTAATATGGCCTGCTGCACCTGCGTGAGGGTAGACTGGAAG TCATCCATGTCCACAAAGCGGTGGTATGCGCCCGTGTTGATGGTGTAAGTGCAGTTGGCCATAACGGACCAGTTAAC GGTCTGGTGACCCGGCTGCGAGAGCTCGGTGTACCTGAGACGCGAGTAAGCCCTCGAGTCAAATACGTAGTCGTTGC AAGTCCGCACCAGGTACTGGTATCCCACCAAAAAGTGCGGCGGCGGCTGGCGGTAGAGGGGCCAGCGTAGGGTGGCC GGGGCTCCGGGGGCGAGATCTTCCAACATAAGGCGATGATATCCGTAGATGTACCTGGACATCCAGGTGATGCCGGC GGCGGTGGTGGAGGCGCGCGGAAAGTCGCGGACGCGGTTCCAGATGTTGCGCAGCGGCAAAAAGTGCTCCATGGTCG GGACGCTCTGGCCGGTCAGGCGCGCGCAATCGTTGACGCTCTAGACCGTGCAAAAGGAGAGCCTGTAAGCGGGCACT CTTCCGTGGTCTGGTGGATAAATTCGCAAGGGTATCATGGCGGACGACCGGGGTTCGAGCCCCGTATCCGGCCGTCC GCCGTGATCCATGCGGTTACCGCCCGCGTGTCGAACCCAGGTGTGCGACGTCAGACAACGGGGGAGTGCTCCTTTTG GCTTCCTTCCAGGCGCGGCGGCTGCTGCGCTAGCTTTTTTGGCCACTGGCCGCGCGCAGCGTAAGCGGTTAGGCTGG AAAGCGAAAGCATTAAGTGGCTCGCTCCCTGTAGCCGGAGGGTTATTTTCCAAGGGTTGAGTCGCGGGACCCCCGGT TCGAGTCTCGGACCGGCCGGACTGCGGCGAACGGGGGTTTGCCTCCCCGTCATGCAAGACCCCGCTTGCAAATTCCT CCGGAAACAGGGACGAGCCCCTTTTTTGCTTTTCCCAGATGCATCCGGTGCTGCGGCAGATGCGCCCCCCTCCTCAG CAGCGGCAAGAGCAAGAGCAGCGGCAGACATGCAGGGCACCCTCCCCTCCTCCTACCGCGTCAGGAGGGGCGACATC CGCGGTTGACGCGGCAGCAGATGGTGATTACGAACCCCCGCGGCGCCGGGCCCGGCACTACCTGGACTTGGAGGAGG GCGAGGGCCTGGCGCGGCTAGGAGCGCCCTCTCCTGAGCGGTACCCAAGGGTGCAGCTGAAGCGTGATACGCGTGAG GCGTACGTGCCGCGGCAGAACCTGTTTCGCGACCGCGAGGGAGAGGAGCCCGAGGAGATGCGGGATCGAAAGTTCCA CGCAGGGCGCGAGCTGCGGCATGGCCTGAATCGCGAGCGGTTGCTGCGCGAGGAGGACTTTGAGCCCGACGCGCGAA CCGGGATTAGTCCCGCGCGCGCACACGTGGCGGCCGCCGACCTGGTAACCGCATACGAGCAGACGGTGAACCAGGAG ATTAACTTTCAAAAAAGCTTTAACAACCACGTGCGTACGCTTGTGGCGCGCGAGGAGGTGGCTATAGGACTGATGCA TCTGTGGGACTTTGTAAGCGCGCTGGAGCAAAACCCAAATAGCAAGCCGCTCATGGCGCAGCTGTTCCTTATAGTGC AGCACAGCAGGGACAACGAGGCATTCAGGGATGCGCTGCTAAACATAGTAGAGCCCGAGGGCCGCTGGCTGCTCGAT TTGATAAACATCCTGCAGAGCATAGTGGTGCAGGAGCGCAGCTTGAGCCTGGCTGACAAGGTGGCCGCCATCAACTA TTCCATGCTTAGCCTGGGCAAGTTTTACGCCCGCAAGATATACCATACCCCTTACGTTCCCATAGACAAGGAGGTAA AGATCGAGGGGTTCTACATGCGCATGGCGCTGAAGGTGCTTACCTTGAGCGACGACCTGGGCGTTTATCGCAACGAG CGCATCCACAAGGCCGTGAGCGTGAGCCGGCGGCGCGAGCTCAGCGACCGCGAGCTGATGCACAGCCTGCAAAGGGC CCTGGCTGGCACGGGCAGCGGCGATAGAGAGGCCGAGTCCTACTTTGACGCGGGCGCTGACCTGCGCTGGGCCCCAA GCCGACGCGCCCTGGAGGCAGCTGGGGCCGGACCTGGGCTGGCGGTGGCACCCGCGCGCGCTGGCAACGTCGGCGGC GTGGAGGAATATGACGAGGACGATGAGTACGAGCCAGAGGACGGCGAGTACTAAGCGGTGATGTTTCTGATCAGATG ATGCAAGACGCAACGGACCCGGCGGTGCGGGCGGCGCTGCAGAGCCAGCCGTCCGGCCTTAACTCCACGGACGACTG GCGCCAGGTCATGGACCGCATCATGTCGCTGACTGCGCGCAATCCTGACGCGTTCCGGCAGCAGCCGCAGGCCAACC GGCTCTCCGCAATTCTGGAAGCGGTGGTCCCGGCGCGCGCAAACCCCACGCACGAGAAGGTGCTGGCGATCGTAAAC GCGCTGGCCGAAAACAGGGCCATCCGGCCCGACGAGGCCGGCCTGGTCTACGACGCGCTGCTTCAGCGCGTGGCTCG TTACAACAGCGGCAACGTGCAGACCAACCTGGACCGGCTGGTGGGGGATGTGCGCGAGGCCGTGGCGCAGCGTGAGC GCGCGCAGCAGCAGGGCAACCTGGGCTCCATGGTTGCACTAAACGCCTTCCTGAGTACACAGCCCGCCAACGTGCCG CGGGGACAGGAGGACTACACCAACTTTGTGAGCGCACTGCGGCTAATGGTGACTGAGACACCGCAAAGTGAGGTGTA CCAGTCTGGGCCAGACTATTTTTTCCAGACCAGTAGACAAGGCCTGCAGACCGTAAACCTGAGCCAGGCTTTCAAAA ACTTGCAGGGGCTGTGGGGGGTGCGGGCTCCCACAGGCGACCGCGCGACCGTGTCTAGCTTGCTGACGCCCAACTCG CGCCTGTTGCTGCTGCTAATAGCGCCCTTCACGGACAGTGGCAGCGTGTCCCGGGACACATACCTAGGTCACTTGCT GACACTGTACCGCGAGGCCATAGGTCAGGCGCATGTGGACGAGCATACTTTCCAGGAGATTACAAGTGTCAGCCGCG CGCTGGGGCAGGAGGACACGGGCAGCCTGGAGGCAACCCTAAACTACCTGCTGACCAACCGGCGGCAGAAGATCCCC TCGTTGCACAGTTTAAACAGCGAGGAGGAGCGCATTTTGCGCTACGTGCAGCAGAGCGTGAGCCTTAACCTGATGCG CGACGGGGTAACGCCCAGCGTGGCGCTGGACATGACCGCGCGCAACATGGAACCGGGCATGTATGCCTCAAACCGGC CGTTTATCAACCGCCTAATGGACTACTTGCATCGCGCGGCCGCCGTGAACCCCGAGTATTTCACCAATGCCATCTTG AACCCGCACTGGCTACCGCCCCCTGGTTTCTACACCGGGGGATTCGAGGTGCCCGAGGGTAACGATGGATTCCTCTG GGACGACATAGACGACAGCGTGTTTTCCCCGCAACCGCAGACCCTGCTAGAGTTGCAACAGCGCGAGCAGGCAGAGG CGGCGCTGCGAAAGGAAAGCTTCCGCAGGCCAAGCAGCTTGTCCGATCTAGGCGCTGCGGCCCCGCGGTCAGATGCT AGTAGCCCATTTCCAAGCTTGATAGGGTCTCTTACCAGCACTCGCACCACCCGCCCGCGCCTGCTGGGCGAGGAGGA GTACCTAAACAACTCGCTGCTGCAGCCGCAGCGCGAAAAAAACCTGCCTCCGGCATTTCCCAACAACGGGATAGAGA GCCTAGTGGACAAGATGAGTAGATGGAAGACGTACGCGCAGGAGCACAGGGACGTGCCAGGCCCGCGCCCGCCCACC CGTCGTCAAAGGCACGACCGTCAGCGGGGTCTGGTGTGGGAGGACGATGACTCGGCAGACGACAGCAGCGTCCTGGA TTTGGGAGGGAGTGGCAACCCGTTTGCGCACCTTCGCCCCAGGCTGGGGAGAATGTTTTAAAAAAAAAAAAGCATGA TGCAAAATAAAAAACTCACCAAGGCCATGGCACCGAGCGTTGGTTTTCTTGTATTCCCCTTAGTATGCGGCGCGCGG CGATGTATGAGGAAGGTCCTCCTCCCTCCTACGAGAGTGTGGTGAGCGCGGCGCCAGTGGCGGCGGCGCTGGGTTCT CCCTTCGATGCTCCCCTGGACCCGCCGTTTGTGCCTCCGCGGTACCTGCGGCCTACCGGGGGGAGAAACAGCATCCG TTACTCTGAGTTGGCACCCCTATTCGACACCACCCGTGTGTACCTGGTGGACAACAAGTCAACGGATGTGGCATCCC TGAACTACCAGAACGACCACAGCAACTTTCTGACCACGGTCATTCAAAACAATGACTACAGCCCGGGGGAGGCAAGC ACACAGACCATCAATCTTGACGACCGGTCGCACTGGGGCGGCGACCTGAAAACCATCCTGCATACCAACATGCCAAA TGTGAACGAGTTCATGTTTACCAATAAGTTTAAGGCGCGGGTGATGGTGTCGCGCTTGCCTACTAAGGACAATCAGG TGGAGCTGAAATACGAGTGGGTGGAGTTCACGCTGCCCGAGGGCAACTACTCCGAGACCATGACCATAGACCTTATG AACAACGCGATCGTGGAGCACTACTTGAAAGTGGGCAGACAGAACGGGGTTCTGGAAAGCGACATCGGGGTAAAGTT TGACACCCGCAACTTCAGACTGGGGTTTGACCCCGTCACTGGTCTTGTCATGCCTGGGGTATATACAAACGAAGCCT TCCATCCAGACATCATTTTGCTGCCAGGATGCGGGGTGGACTTCACCCACAGCCGCCTGAGCAACTTGTTGGGCATC CGCAAGCGGCAACCCTTCCAGGAGGGCTTTAGGATCACCTACGATGATCTGGAGGGTGGTAACATTCCCGCACTGTT GGATGTGGACGCCTACCAGGCGAGCTTGAAAGATGACACCGAACAGGGGGGGGGTGGCGCAGGCGGCAGCAACAGCA GTGGCAGCGGCGCGGAAGAGAACTCCAACGCGGCAGCCGCGGCAATGCAGCCGGTGGAGGACATGAACGATCATGCC ATTCGCGGCGACACCTTTGCCACACGGGCTGAGGAGAAGCGCGCTGAGGCCGAAGCAGCGGCCGAAGCTGCCGCCCC CGCTGCGCAACCCGAGGTCGAGAAGCCTCAGAAGAAACCGGTGATCAAACCCCTGACAGAGGACAGCAAGAAACGCA GTTACAACCTAATAAGCAATGACAGCACCTTCACCCAGTACCGCAGCTGGTACCTTGCATACAACTACGGCGACCCT CAGACCGGAATCCGCTCATGGACCCTGCTTTGCACTCCTGACGTAACCTGCGGCTCGGAGCAGGTCTACTGGTCGTT GCCAGACATGATGCAAGACCCCGTGACCTTCCGCTCCACGCGCCAGATCAGCAACTTTCCGGTGGTGGGCGCCGAGC TGTTGCCCGTGCACTCCAAGAGCTTCTACAACGACCAGGCCGTCTACTCCCAACTCATCCGCCAGTTTACCTCTCTG ACCCACGTGTTCAATCGCTTTCCCGAGAACCAGATTTTGGCGCGCCCGCCAGCCCCCACCATCACCACCGTCAGTGA AAACGTTCCTGCTCTCACAGATCACGGGACGCTACCGCTGCGCAACAGCATCGGAGGAGTCCAGCGAGTGACCATTA CTGACGCCAGACGCCGCACCTGCCCCTACGTTTACAAGGCCCTGGGCATAGTCTCGCCGCGCGTCCTATCGAGCCGC ACTTTTTGAGCAAGCATGTCCATCCTTATATCGCCCAGCAATAACACAGGCTGGGGCCTGCGCTTCCCAAGCAAGAT GTTTGGCGGGGCCAAGAAGCGCTCCGACCAACACCCAGTGCGCGTGCGCGGGCACTACCGCGCGCCCTGGGGCGCGC ACAAACGCGGCCGCACTGGGCGCACCACCGTCGATGACGCCATCGACGCGGTGGTGGAGGAGGCGCGCAACTACACG CCCACGCCGCCACCAGTGTCCACAGTGGACGCGGCCATTCAGACCGTGGTGCGCGGAGCCCGGCGCTATGCTAAAAT GAAGAGACGGCGGAGGCGCGTAGCACGTCGCCACCGCCGCCGACCCGGCACTGCCGCCCAACGCGCGGCGGCGGCCC TGCTTAACCGCGCACGTCGCACCGGCCGACGGGCGGCCATGCGGGCCGCTCGAAGGCTGGCCGCGGGTATTGTCACT GTGCCCCCCAGGTCCAGGCGACGAGCGGCCGCCGCAGCAGCCGCGGCCATTAGTGCTATGACTCAGGGTCGCAGGGG CAACGTGTATTGGGTGCGCGACTCGGTTAGCGGCCTGCGCGTGCCCGTGCGCACCCGCCCCCCGCGCAACTAGATTG CAAGAAAAAACTACTTAGACTCGTACTGTTGTATGTATCCAGCGGCGGCGGCGCGCAACGAAGCTATGTCCAAGCGC AAAATCAAAGAAGAGATGCTCCAGGTCATCGCGCCGGAGATCTATGGCCCCCCGAAGAAGGAAGAGCAGGATTACAA GCCCCGAAAGCTAAAGCGGGTCAAAAAGAAAAAGAAAGATGATGATGATGAACTTGACGACGAGGTGGAACTGCTGC ACGCTACCGCGCCCAGGCGACGGGTACAGTGGAAAGGTCGACGCGTAAAACGTGTTTTGCGACCCGGCACCACCGTA GTCTTTACGCCCGGTGAGCGCTCCACCCGCACCTACAAGCGCGTGTATGATGAGGTGTACGGCGACGAGGACCTGCT TGAGCAGGCCAACGAGCGCCTCGGGGAGTTTGCCTACGGAAAGCGGCATAAGGACATGCTGGCGTTGCCGCTGGACG AGGGCAACCCAACACCTAGCCTAAAGCCCGTAACACTGCAGCAGGTGCTGCCCGCGCTTGCACCGTCCGAAGAAAAG CGCGGCCTAAAGCGCGAGTCTGGTGACTTGGCACCCACCGTGCAGCTGATGGTACCCAAGCGCCAGCGACTGGAAGA TGTCTTGGAAAAAATGACCGTGGAACCTGGGCTGGAGCCCGAGGTCCGCGTGCGGCCAATCAAGCAGGTGGCGCCGG GACTGGGCGTGCAGACCGTGGACGTTCAGATACCCACTACCAGTAGCACCAGTATTGCCACCGCCACAGAGGGCATG GAGACACAAACGTCCCCGGTTGCCTCAGCGGTGGCGGATGCCGCGGTGCAGGCGGTCGCTGCGGCCGCGTCCAAGAC CTCTACGGAGGTGCAAACGGACCCGTGGATGTTTCGCGTTTCAGCCCCCCGGCGCCCGCGCGGTTCGAGGAAGTACG GCGCCGCCAGCGCGCTACTGCCCGAATATGCCCTACATCCTTCCATTGCGCCTACCCCCGGCTATCGTGGCTACACC TACCGCCCCAGAAGACGAGCAACTACCCGACGCCGAACCACCACTGGAACCCGCCGCCGCCGTCGCCGTCGCCAGCC CGTGCTGGCCCCGATTTCCGTGCGCAGGGTGGCTCGCGAAGGAGGCAGGACCCTGGTGCTGCCAACAGCGCGCTACC ACCCCAGCATCGTTTAAAAGCCGGTCTTTGTGGTTCTTGCAGATATGGCCCTCACCTGCCGCCTCCGTTTCCCGGTG CCGGGATTCCGAGGAAGAATGCACCGTAGGAGGGGCATGGCCGGCCACGGCCTGACGGGCGGCATGCGTCGTGCGCA CCACCGGCGGCGGCGCGCGTCGCACCGTCGCATGCGCGGCGGTATCCTGCCCCTCCTTATTCCACTGATCGCCGCGG CGATTGGCGCCGTGCCCGGAATTGCATCCGTGGCCTTGCAGGCGCAGAGACACTGATTAAAAACAAGTTGCATGTGG AAAAATCAAAATAAAAAGTCTGGACTCTCACGCTCGCTTGGTCCTGTAACTATTTTGTAGAATGGAAGACATCAACT TTGCGTCTCTGGCCCCGCGACACGGCTCGCGCCCGTTCATGGGAAACTGGCAAGATATCGGCACCAGCAATATGAGC GGTGGCGCCTTCAGCTGGGGCTCGCTGTGGAGCGGCATTAAAAATTTCGGTTCCACCGTTAAGAACTATGGCAGCAA GGCCTGGAACAGCAGCACAGGCCAGATGCTGAGGGATAAGTTGAAAGAGCAAAATTTCCAACAAAAGGTGGTAGATG GCCTGGCCTCTGGCATTAGCGGGGTGGTGGACCTGGCCAACCAGGCAGTGCAAAATAAGATTAACAGTAAGCTTGAT CCCCGCCCTCCCGTAGAGGAGCCTCCACCGGCCGTGGAGACAGTGTCTCCAGAGGGGCGTGGCGAAAAGCGTCCGCG CCCCGACAGGGAAGAAACTCTGGTGACGCAAATAGACGAGCCTCCCTCGTACGAGGAGGCACTAAAGCAAGGCCTGC CCACCACCCGTCCCATCGCGCCCATGGCTACCGGAGTGCTGGGCCAGCACACACCCGTAACGCTGGACCTGCCTCCC CCCGCCGACACCCAGCAGAAACCTGTGCTGCCAGGCCCGACCGCCGTTGTTGTAACCCGTCCTAGCCGCGCGTCCCT GCGCCGCGCCGCCAGCGGTCCGCGATCGTTGCGGCCCGTAGCCAGTGGCAACTGGCAAAGCACACTGAACAGCATCG TGGGTCTGGGGGTGCAATCCCTGAAGCGCCGACGATGCTTCTGAATAGCTAACGTGTCGTATGTGTGTCATGTATGC GTCCATGTCGCCGCCAGAGGAGCTGCTGAGCCGCCGCGCGCCCGCTTTCCAAGATGGCTACCCCTTCGATGATGCCG CAGTGGTCTTACATGCACATCTCGGGCCAGGACGCCTCGGAGTACCTGAGCCCCGGGCTGGTGCAGTTTGCCCGCGC CACCGAGACGTACTTCAGCCTGAATAACAAGTTTAGAAACCCCACGGTGGCGCCTACGCACGACGTGACCACAGACC GGTCCCAGCGTTTGACGCTGCGGTTCATCCCTGTGGACCGTGAGGATACTGCGTACTCGTACAAGGCGCGGTTCACC CTAGCTGTGGGTGATAACCGTGTGCTGGACATGGCTTCCACGTACTTTGACATCCGCGGCGTGCTGGACAGGGGCCC TACTTTTAAGCCCTACTCTGGCACTGCCTACAACGCCCTGGCTCCCAAGGGTGCCCCAAATCCTTGCGAATGGGATG AAGCTGCTACTGCTCTTGAAATAAACCTAGAAGAAGAGGACGATGACAACGAAGACGAAGTAGACGAGCAAGCTGAG CAGCAAAAAACTCACGTATTTGGGCAGGCGCCTTATTCTGGTATAAATATTACAAAGGAGGGTATTCAAATAGGTGT CGAAGGTCAAACACCTAAATATGCCGATAAAACATTTCAACCTGAACCTCAAATAGGAGAATCTCAGTGGTACGAAA CTGAAATTAATCATGCAGCTGGGAGAGTCCTTAAAAAGACTACCCCAATGAAACCATGTTACGGTTCATATGCAAAA CCCACAAATGAAAATGGAGGGCAAGGCATTCTTGTAAAGCAACAAAATGGAAAGCTAGAAAGTCAAGTGGAAATGCA ATTTTTCTCAACTACTGAGGCGACCGCAGGCAATGGTGATAACTTGACTCCTAAAGTGGTATTGTACAGTGAAGATG TAGATATAGAAACCCCAGACACTCATATTTCTTACATGCCCACTATTAAGGAAGGTAACTCACGAGAACTAATGGGC CAACAATCTATGCCCAACAGGCCTAATTACATTGCTTTTAGGGACAATTTTATTGGTCTAATGTATTACAACAGCAC GGGTAATATGGGTGTTCTGGCGGGCCAAGCATCGCAGTTGAATGCTGTTGTAGATTTGCAAGACAGAAACACAGAGC TTTCATACCAGCTTTTGCTTGATTCCATTGGTGATAGAACCAGGTACTTTTCTATGTGGAATCAGGCTGTTGACAGC TATGATCCAGATGTTAGAATTATTGAAAATCATGGAACTGAAGATGAACTTCCAAATTACTGCTTTCCACTGGGAGG TGTGATTAATACAGAGACTCTTACCAAGGTAAAACCTAAAACAGGTCAGGAAAATGGATGGGAAAAAGATGCTACAG AATTTTCAGATAAAAATGAAATAAGAGTTGGAAATAATTTTGCCATGGAAATCAATCTAAATGCCAACCTGTGGAGA AATTTCCTGTACTCCAACATAGCGCTGTATTTGCCCGACAAGCTAAAGTACAGTCCTTCCAACGTAAAAATTTCTGA TAACCCAAACACCTACGACTACATGAACAAGCGAGTGGTGGCTCCCGGGTTAGTGGACTGCTACATTAACCTTGGAG CACGCTGGTCCCTTGACTATATGGACAACGTCAACCCATTTAACCACCACCGCAATGCTGGCCTGCGCTACCGCTCA ATGTTGCTGGGCAATGGTCGCTATGTGCCCTTCCACATCCAGGTGCCTCAGAAGTTCTTTGCCATTAAAAACCTCCT TCTCCTGCCGGGCTCATACACCTACGAGTGGAACTTCAGGAAGGATGTTAACATGGTTCTGCAGAGCTCCCTAGGAA ATGACCTAAGGGTTGACGGAGCCAGCATTAAGTTTGATAGCATTTGCCTTTACGCCACCTTCTTCCCCATGGCCCAC AACACCGCCTCCACGCTTGAGGCCATGCTTAGAAACGACACCAACGACCAGTCCTTTAACGACTATCTCTCCGCCGC CAACATGCTCTACCCTATACCCGCCAACGCTACCAACGTGCCCATATCCATCCCCTCCCGCAACTGGGCGGCTTTCC GCGGCTGGGCCTTCACGCGCCTTAAGACTAAGGAAACCCCATCACTGGGCTCGGGCTACGACCCTTATTACACCTAC TCTGGCTCTATACCCTACCTAGATGGAACCTTTTACCTCAACCACACCTTTAAGAAGGTGGCCATTACCTTTGACTC TTCTGTCAGCTGGCCTGGCAATGACCGCCTGCTTACCCCCAACGAGTTTGAAATTAAGCGCTCAGTTGACGGGGAGG GTTACAACGTTGCCCAGTGTAACATGACCAAAGACTGGTTCCTGGTACAAATGCTAGCTAACTACAACATTGGCTAC CAGGGCTTCTATATCCCAGAGAGCTACAAGGACCGCATGTACTCCTTCTTTAGAAACTTCCAGCCCATGAGCCGTCA GGTGGTGGATGATACTAAATACAAGGACTACCAACAGGTGGGCATCCTACACCAACACAACAACTCTGGATTTGTTG GCTACCTTGCCCCCACCATGCGCGAAGGACAGGCCTACCCTGCTAACTTCCCCTATCCGCTTATAGGCAAGACCGCA GTTGACAGCATTACCCAGAAAAAGTTTCTTTGCGATCGCACCCTTTGGCGCATCCCATTCTCCAGTAACTTTATGTC CATGGGCGCACTCACAGACCTGGGCCAAAACCTTCTCTACGCCAACTCCGCCCACGCGCTAGACATGACTTTTGAGG TGGATCCCATGGACGAGCCCACCCTTCTTTATGTTTTGTTTGAAGTCTTTGACGTGGTCCGTGTGCACCGGCCGCAC CGCGGCGTCATCGAAACCGTGTACCTGCGCACGCCCTTCTCGGCCGGCAACGCCACAACATAAAGAAGCAAGCAACA TCAACAACAGCTGCCGCCATGGGCTCCAGTGAGCAGGAACTGAAAGCCATTGTCAAAGATCTTGGTTGTGGGCCATA TTTTTTGGGCACCTATGACAAGCGCTTTCCAGGCTTTGTTTCTCCACACAAGCTCGCCTGCGCCATAGTCAATACGG CCGGTCGCGAGACTGGGGGCGTACACTGGATGGCCTTTGCCTGGAACCCGCACTCAAAAACATGCTACCTCTTTGAG CCCTTTGGCTTTTCTGACCAGCGACTCAAGCAGGTTTACCAGTTTGAGTACGAGTCACTCCTGCGCCGTAGCGCCAT TGCTTCTTCCCCCGACCGCTGTATAACGCTGGAAAAGTCCACCCAAAGCGTACAGGGGCCCAACTCGGCCGCCTGTG GACTATTCTGCTGCATGTTTCTCCACGCCTTTGCCAACTGGCCCCAAACTCCCATGGATCACAACCCCACCATGAAC CTTATTACCGGGGTACCCAACTCCATGCTCAACAGTCCCCAGGTACAGCCCACCCTGCGTCGCAACCAGGAACAGCT CTACAGCTTCCTGGAGCGCCACTCGCCCTACTTCCGCAGCCACAGTGCGCAGATTAGGAGCGCCACTTCTTTTTGTC ACTTGAAAAACATGTAAAAATAATGTACTAGAGACACTTTCAATAAAGGCAAATGCTTTTATTTGTACACTCTCGGG TGATTATTTACCCCCACCCTTGCCGTCTGCGCCGTTTAAAAATCAAAGGGGTTCTGCCGCGCATCGCTATGCGCCAC TGGCAGGGACACGTTGCGATACTGGTGTTTAGTGCTCCACTTAAACTCAGGCACAACCATCCGCGGCAGCTCGGTGA AGTTTTCACTCCACAGGCTGCGCACCATCACCAACGCGTTTAGCAGGTCGGGCGCCGATATCTTGAAGTCGCAGTTG GGGCCTCCGCCCTGCGCGCGCGAGTTGCGATACACAGGGTTGCAGCACTGGAACACTATCAGCGCCGGGTGGTGCAC GCTGGCCAGCACGCTCTTGTCGGAGATCAGATCCGCGTCCAGGTCCTCCGCGTTGCTCAGGGCGAACGGAGTCAACT TTGGTAGCTGCCTTCCCAAAAAGGGCGCGTGCCCAGGCTTTGAGTTGCACTCGCACCGTAGTGGCATCAAAAGGTGA CCGTGCCCGGTCTGGGCGTTAGGATACAGCGCCTGCATAAAAGCCTTGATCTGCTTAAAAGCCACCTGAGCCTTTGC GCCTTCAGAGAAGAACATGCCGCAAGACTTGCCGGAAAACTGATTGGCCGGACAGGCCGCGTCGTGCACGCAGCACC TTGCGTCGGTGTTGGAGATCTGCACCACATTTCGGCCCCACCGGTTCTTCACGATCTTGGCCTTGCTAGACTGCTCC TTCAGCGCGCGCTGCCCGTTTTCGCTCGTCACATCCATTTCAATCACGTGCTCCTTATTTATCATAATGCTTCCGTG TAGACACTTAAGCTCGCCTTCGATCTCAGCGCAGCGGTGCAGCCACAACGCGCAGCCCGTGGGCTCGTGATGCTTGT AGGTCACCTCTGCAAACGACTGCAGGTACGCCTGCAGGAATCGCCCCATCATCGTCACAAAGGTCTTGTTGCTGGTG AAGGTCAGCTGCAACCCGCGGTGCTCCTCGTTCAGCCAGGTCTTGCATACGGCCGCCAGAGCTTCCACTTGGTCAGG CAGTAGTTTGAAGTTCGCCTTTAGATCGTTATCCACGTGGTACTTGTCCATCAGCGCGCGCGCAGCCTCCATGCCCT TCTCCCACGCAGACACGATCGGCACACTCAGCGGGTTCATCACCGTAATTTCACTTTCCGCTTCGCTGGGCTCTTCC TCTTCCTCTTGCGTCCGCATACCACGCGCCACTGGGTCGTCTTCATTCAGCCGCCGCACTGTGCGCTTACCTCCTTT GCCATGCTTGATTAGCACCGGTGGGTTGCTGAAACCCACCATTTGTAGCGCCACATCTTCTCTTTCTTCCTCGCTGT CCACGATTACCTCTGGTGATGGCGGGCGCTCGGGCTTGGGAGAAGGGCGCTTCTTTTTCTTCTTGGGCGCAATGGCC AAATCCGCCGCCGAGGTCGATGGCCGCGGGCTGGGTGTGCGCGGCACCAGCGCGTCTTGTGATGAGTCTTCCTCGTC CTCGGACTCGATACGCCGCCTCATCCGCTTTTTTGGGGGCGCCCGGGGAGGCGGCGGCGACGGGGACGGGGACGACA CGTCCTCCATGGTTGGGGGACGTCGCGCCGCACCGCGTCCGCGCTCGGGGGTGGTTTCGCGCTGCTCCTCTTCCCGA CTGGCCATTTCCTTCTCCTATAGGCAGAAAAAGATCATGGAGTCAGTCGAGAAGAAGGACAGCCTAACCGCCCCCTC TGAGTTCGCCACCACCGCCTCCACCGATGCCGCCAACGCGCCTACCACCTTCCCCGTCGAGGCACCCCCGCTTGAGG AGGAGGAAGTGATTATCGAGCAGGACCCAGGTTTTGTAAGCGAAGACGACGAGGACCGCTCAGTACCAACAGAGGAT AAAAAGCAAGACCAGGACAACGCAGAGGCAAACGAGGAACAAGTCGGGCGGGGGGACGAAAGGCATGGCGACTACCT AGATGTGGGAGACGACGTGCTGTTGAAGCATCTGCAGCGCCAGTGCGCCATTATCTGCGACGCGTTGCAAGAGCGCA GCGATGTGCCCCTCGCCATAGCGGATGTCAGCCTTGCCTACGAACGCCACCTATTCTCACCGCGCGTACCCCCCAAA CGCCAAGAAAACGGCACATGCGAGCCCAACCCGCGCCTCAACTTCTACCCCGTATTTGCCGTGCCAGAGGTGCTTGC CACCTATCACATCTTTTTCCAAAACTGCAAGATACCCCTATCCTGCCGTGCCAACCGCAGCCGAGCGGACAAGCAGC TGGCCTTGCGGCAGGGCGCTGTCATACCTGATATCGCCTCGCTCAACGAAGTGCCAAAAATCTTTGAGGGTCTTGGA CGCGACGAGAAGCGCGCGGCAAACGCTCTGCAACAGGAAAACAGCGAAAATGAAAGTCACTCTGGAGTGTTGGTGGA ACTCGAGGGTGACAACGCGCGCCTAGCCGTACTAAAACGCAGCATCGAGGTCACCCACTTTGCCTACCCGGCACTTA ACCTACCCCCCAAGGTCATGAGCACAGTCATGAGTGAGCTGATCGTGCGCCGTGCGCAGCCCCTGGAGAGGGATGCA AATTTGCAAGAACAAACAGAGGAGGGCCTACCCGCAGTTGGCGACGAGCAGCTAGCGCGCTGGCTTCAAACGCGCGA GCCTGCCGACTTGGAGGAGCGACGCAAACTAATGATGGCCGCAGTGCTCGTTACCGTGGAGCTTGAGTGCATGCAGC GGTTCTTTGCTGACCCGGAGATGCAGCGCAAGCTAGAGGAAACATTGCACTACACCTTTCGACAGGGCTACGTACGC CAGGCCTGCAAGATCTCCAACGTGGAGCTCTGCAACCTGGTCTCCTACCTTGGAATTTTGCACGAAAACCGCCTTGG GCAAAACGTGCTTCATTCCACGCTCAAGGGCGAGGCGCGCCGCGACTACGTCCGCGACTGCGTTTACTTATTTCTAT GCTACACCTGGCAGACGGCCATGGGCGTTTGGCAGCAGTGCTTGGAGGAGTGCAACCTCAAGGAGCTGCAGAAACTG CTAAAGCAAAACTTGAAGGACCTATGGACGGCCTTCAACGAGCGCTCCGTGGCCGCGCACCTGGCGGACATCATTTT CCCCGAACGCCTGCTTAAAACCCTGCAACAGGGTCTGCCAGACTTCACCAGTCAAAGCATGTTGCAGAACTTTAGGA ACTTTATCCTAGAGCGCTCAGGAATCTTGCCCGCCACCTGCTGTGCACTTCCTAGCGACTTTGTGCCCATTAAGTAC CGCGAATGCCCTCCGCCGCTTTGGGGCCACTGCTACCTTCTGCAGCTAGCCAACTACCTTGCCTACCACTCTGACAT AATGGAAGACGTGAGCGGTGACGGTCTACTGGAGTGTCACTGTCGCTGCAACCTATGCACCCCGCACCGCTCCCTGG TTTGCAATTCGCAGCTGCTTAACGAAAGTCAAATTATCGGTACCTTTGAGCTGCAGGGTCCCTCGCCTGACGAAAAG TCCGCGGCTCCGGGGTTGAAACTCACTCCGGGGCTGTGGACGTCGGCTTACCTTCGCAAATTTGTACCTGAGGACTA CCACGCCCACGAGATTAGGTTCTACGAAGACCAATCCCGCCCGCCAAATGCGGAGCTTACCGCCTGCGTCATTACCC AGGGCCACATTCTTGGCCAATTGCAAGCCATCAACAAAGCCCGCCAAGAGTTTCTGCTACGAAAGGGACGGGGGGTT TACTTGGACCCCCAGTCCGGCGAGGAGCTCAACCCAATCCCCCCGCCGCCGCAGCCCTATCAGCAGCAGCCGCGGGC CCTTGCTTCCCAGGATGGCACCCAAAAAGAAGCTGCAGCTGCCGCCGCCACCCACGGACGAGGAGGAATACTGGGAC AGTCAGGCAGAGGAGGTTTTGGACGAGGAGGAGGAGGACATGATGGAAGACTGGGAGAGCCTAGACGAGGAAGCTTC CGAGGTCGAAGAGGTGTCAGACGAAACACCGTCACCCTCGGTCGCATTCCCCTCGCCGGCGCCCCAGAAATCGGCAA CCGGTTCCAGCATGGCTACAACCTCCGCTCCTCAGGCGCCGCCGGCACTGCCCGTTCGCCGACCCAACCGTAGATGG GACACCACTGGAACCAGGGCCGGTAAGTCCAAGCAGCCGCCGCCGTTAGCCCAAGAGCAACAACAGCGCCAAGGCTA CCGCTCATGGCGCGGGCACAAGAACGCCATAGTTGCTTGCTTGCAAGACTGTGGGGGCAACATCTCCTTCGCCCGCC GCTTTCTTCTCTACCATCACGGCGTGGCCTTCCCCCGTAACATCCTGCATTACTACCGTCATCTCTACAGCCCATAC TGCACCGGCGGCAGCGGCAGCGGCAGCAACAGCAGCGGCCACACAGAAGCAAAGGCGACCGGATAGCAAGACTCTGA CAAAGCCCAAGAAATCCACAGCGGCGGCAGCAGCAGGAGGAGGAGCGCTGCGTCTGGCGCCCAACGAACCCGTATCG ACCCGCGAGCTTAGAAACAGGATTTTTCCCACTCTGTATGCTATATTTCAACAGAGCAGGGGCCAAGAACAAGAGCT GAAAATAAAAAACAGGTCTCTGCGATCCCTCACCCGCAGCTGCCTGTATCACAAAAGCGAAGATCAGCTTCGGCGCA CGCTGGAAGACGCGGAGGCTCTCTTCAGTAAATACTGCGCGCTGACTCTTAAGGACTAGTTTCGCGCCCTTTCTCAA ATTTAAGCGCGAAAACTACGTCATCTCCAGCGGCCACACCCGGCGCCAGCACCTGTCGTCAGCGCCATTATGAGCAA GGAAATTCCCACGCCCTACATGTGGAGTTACCAGCCACAAATGGGACTTGCGGCTGGAGCTGCCCAAGACTACTCAA CCCGAATAAACTACATGAGCGCGGGACCCCACATGATATCCCGGGTCAACGGAATCCGCGCCCACCGAAACCGAATT CTCTTGGAACAGGCGGCTATTACCACCACACCTCGTAATAACCTTAATCCCCGTAGTTGGCCCGCTGCCCTGGTGTA CCAGGAAAGTCCCGCTCCCACCACTGTGGTACTTCCCAGAGACGCCCAGGCCGAAGTTCAGATGACTAACTCAGGGG CGCAGCTTGCGGGCGGCTTTCGTCACAGGGTGCGGTCGCCCGGGCAGGGTATAACTCACCTGACAATCAGAGGGCGA GGTATTCAGCTCAACGACGAGTCGGTGAGCTCCTCGCTTGGTCTCCGTCCGGACGGGACATTTCAGATCGGCGGCGC CGGCCGTCCTTCATTCACGCCTCGTCAGGCAATCCTAACTCTGCAGACCTCGTCCTCTGAGCCGCGCTCTGGAGGCA TTGGAACTCTGCAATTTATTGAGGAGTTTGTGCCATCGGTCTACTTTAACCCCTTCTCGGGACCTCCCGGCCACTAT CCGGATCAATTTATTCCTAACTTTGACGCGGTAAAGGACTCGGCGGACGGCTACGACTGAATGTTAAGTGGAGAGGC AGAGCAACTGCGCCTGAAACACCTGGTCCACTGTCGCCGCCACAAGTGCTTTGCCCGCGACTCCGGTGAGTTTTGCT ACTTTGAATTGCCCGAGGATCATATCGAGGGCCCGGCGCACGGCGTCCGGCTTACCGCCCAGGGAGAGCTTGCCCGT AGCCTGATTCGGGAGTTTACCCAGCGCCCCCTGCTAGTTGAGCGGGACAGGGGACCCTGTGTTCTCACTGTGATTTG CAACTGTCCTAACCTTGGATTACATCAAGATCTTTGTTGCCATCTCTGTGCTGAGTATAATAAATACAGAAATTAAA ATATACTGGGGCTCCTATCGCCATCCTGTAAACGCCACCGTCTTCACCCGCCCAAGCAAACCAAGGCGAACCTTACC TGGTACTTTTAACATCTCTCCCTCTGTGATTTACAACAGTTTCAACCCAGACGGAGTGAGTCTACGAGAGAACCTCT CCGAGCTCAGCTACTCCATCAGAAAAAACACCACCCTCCTTACCTGCCGGGAACGTACGAGTGCGTCACCGGCCGCT GCACCACACCTACCGCCTGACCGTAAACCAGACTTTTTCCGGACAGACCTCAATAACTCTGTTTACCAGAACAGGAG GTGAGCTTAGAAAACCCTTAGGGTATTAGGCCAAAGGCGCAGCTACTGTGGGGTTTATGAACAATTCAAGCAACTCT ACGGGCTATTCTAATTCAGGTTTCTCTAGAATCGGGGTTGGGGTTATTCTCTGTCTTGTGATTCTCTTTATTCTTAT ACTAACGCTTCTCTGCCTAAGGCTCGCCGCCTGCTGTGTGCACATTTGCATTTATTGTCAGCTTTTTAAACGCTGGG GTCGCCACCCAAGATGATTAGGTACATAATCCTAGGTTTACTCACCCTTGCGTCAGCCCACGGTACCACCCAAAAGG TGGATTTTAAGGAGCCAGCCTGTAATGTTACATTCGCAGCTGAAGCTAATGAGTGCACCACTCTTATAAAATGCACC ACAGAACATGAAAAGCTGCTTATTCGCCACAAAAACAAAATTGGCAAGTATGCTGTTTATGCTATTTGGCAGCCAGG TGACACTACAGAGTATAATGTTACAGTTTTCCAGGGTAAAAGTCATAAAACTTTTATGTATACTTTTCCATTTTATG AAATGTGCGACATTACCATGTACATGAGCAAACAGTATAAGTTGTGGCCCCCACAAAATTGTGTGGAAAACACTGGC ACTTTCTGCTGCACTGCTATGCTAATTACAGTGCTCGCTTTGGTCTGTACCCTACTCTATATTAAATACAAAAGCAG ACGCAGCTTTATTGAGGAAAAGAAAATGCCTTAATTTACTAAGTTACAAAGCTAATGTCACCACTAACTGCTTTACT CGCTGCTTGCAAAACAAATTCAAAAAGTTAGCATTATAATTAGAATAGGATTTAAACCCCCCGGTCATTTCCTGCTC AATACCATTCCCCTGAACAATTGACTCTATGTGGGATATGCTCCAGCGCTACAACCTTGAAGTCAGGCTTCCTGGAT GTCAGCATCTGACTTTGGCCAGCACCTGTCCCGCGGATTTGTTCCAGTCCAACTACAGCGACCCACCCTAACAGAGA TGACCAACACAACCAACGCGGCCGCCGCTACCGGACTTACATCTACCACAAATACACCCCAAGTTTCTGCCTTTGTC AATAACTGGGATAACTTGGGCATGTGGTGGTTCTCCATAGCGCTTATGTTTGTATGCCTTATTATTATGTGGCTCAT CTGCTGCCTAAAGCGCAAACGCGCCCGACCACCCATCTATAGTCCCATCATTGTGCTACACCCAAACAATGATGGAA TCCATAGATTGGACGGACTGAAACACATGTTCTTTTCTCTTACAGTATGATTAAATGAGACATGATTCCTCGAGTTT TTATATTACTGACCCTTGTTGCGCTTTTTTGTGCGTGCTCCACATTGGCTGCGGTTTCTCACATCGAAGTAGACTGC ATTCCAGCCTTCACAGTCTATTTGCTTTACGGATTTGTCACCCTCACGCTCATCTGCAGCCTCATCACTGTGGTCAT CGCCTTTATCCAGTGCATTGACTGGGTCTGTGTGCGCTTTGCATATCTCAGACACCATCCCCAGTACAGGGACAGGA CTATAGCTGAGCTTCTTAGAATTCTTTAATTATGAAATTTACTGTGACTTTTCTGCTGATTATTTGCACCCTATCTG CGTTTTGTTCCCCGACCTCCAAGCCTCAAAGACATATATCATGCAGATTCACTCGTATATGGAATATTCCAAGTTGC TACAATGAAAAAAGCGATCTTTCCGAAGCCTGGTTATATGCAATCATCTCTGTTATGGTGTTCTGCAGTACCATCTT AGCCCTAGCTATATATCCCTACCTTGACATTGGCTGGAAACGAATAGATGCCATGAACCACCCAACTTTCCCCGCGC CCGCTATGCTTCCACTGCAACAAGTTGTTGCCGGCGGCTTTGTCCCAGCCAATCAGCCTCGCCCCACTTCTCCCACC CCCACTGAAATCAGCTACTTTAATCTAACAGGAGGAGATGACTGACACCCTAGATCTAGAAATGGACGGAATTATTA CAGAGCAGCGCCTGCTAGAAAGACGCAGGGCAGCGGCCGAGCAACAGCGCATGAATCAAGAGCTCCAAGACATGGTT AACTTGCACCAGTGCAAAAGGGGTATCTTTTGTCTGGTAAAGCAGGCCAAAGTCACCTACGACAGTAATACCACCGG ACACCGCCTTAGCTACAAGTTGCCAACCAAGCGTCAGAAATTGGTGGTCATGGTGGGAGAAAAGCCCATTACCATAA CTCAGCACTCGGTAGAAACCGAAGGCTGCATTCACTCACCTTGTCAAGGACCTGAGGATCTCTGCACCCTTATTAAG ACCCTGTGCGGTCTCAAAGATCTTATTCCCTTTAACTAATAAAAAAAAATAATAAAGCATCACTTACTTAAAATCAG TTAGCAAATTTCTGTCCAGTTTATTCAGCAGCACCTCCTTGCCCTCCTCCCAGCTCTGGTATTGCAGCTTCCTCCTG GCTGCAAACTTTCTCCACAATCTAAATGGAATGTCAGTTTCCTCCTGTTCCTGTCCATCCGCACCCACTATCTTCAT GTTGTTGCAGATGAAGCGCGCAAGACCGTCTGAAGATACCTTCAACCCCGTGTATCCATATGACACGGAAACCGGTC CTCCAACTGTGCCTTTTCTTACTCCTCCCTTTGTATCCCCCAATGGGTTTCAAGAGAGTCCCCCTGGGGTACTCTCT TTGCGCCTATCCGAACCTCTAGTTACCTCCAATGGCATGCTTGCGCTCAAAATGGGCAACGGCCTCTCTCTGGACGA GGCCGGCAACCTTACCTCCCAAAATGTAACCACTGTGAGCCCACCTCTCAAAAAAACCAAGTCAAACATAAACCTGG AAATATCTGCACCCCTCACAGTTACCTCAGAAGCCCTAACTGTGGCTGCCGCCGCACCTCTAATGGTCGCGGGCAAC ACACTCACCATGCAATCACAGGCCCCGCTAACCGTGCACGACTCCAAACTTAGCATTGCCACCCAAGGACCCCTCAC AGTGTCAGAAGGAAAGCTAGCCCTGCAAACATCAGGCCCCCTCACCACCACCGATAGCAGTACCCTTACTATCACTG CCTCACCCCCTCTAACTACTGCCACTGGTAGCTTGGGCATTGACTTGAAAGAGCCCATTTATACACAAAATGGAAAA CTAGGACTAAAGTACGGGGCTCCTTTGCATGTAACAGACGACCTAAACACTTTGACCGTAGCAACTGGTCCAGGTGT GACTATTAATAATACTTCCTTGCAAACTAAAGTTACTGGAGCCTTGGGTTTTGATTCACAAGGCAATATGCAACTTA ATGTAGCAGGAGGACTAAGGATTGATTCTCAAAACAGACGCCTTATACTTGATGTTAGTTATCCGTTTGATGCTCAA AACCAACTAAATCTAAGACTAGGACAGGGCCCTCTTTTTATAAACTCAGCCCACAACTTGGATATTAACTACAACAA AGGCCTTTACTTGTTTACAGCTTCAAACAATTCCAAAAAGCTTGAGGTTAACCTAAGCACTGCCAAGGGGTTGATGT TTGACGCTACAGCCATAGCCATTAATGCAGGAGATGGGCTTGAATTTGGTTCACCTAATGCACCAAACACAAATCCC CTCAAAACAAAAATTGGCCATGGCCTAGAATTTGATTCAAACAAGGCTATGGTTCCTAAACTAGGAACTGGCCTTAG TTTTGACAGCACAGGTGCCATTACAGTAGGAAACAAAAATAATGATAAGCTAACTTTGTGGACCACACCAGCTCCAT CTCCTAACTGTAGACTAAATGCAGAGAAAGATGCTAAACTCACTTTGGTCTTAACAAAATGTGGCAGTCAAATACTT GCTACAGTTTCAGTTTTGGCTGTTAAAGGCAGTTTGGCTCCAATATCTGGAACAGTTCAAAGTGCTCATCTTATTAT AAGATTTGACGAAAATGGAGTGCTACTAAACAATTCCTTCCTGGACCCAGAATATTGGAACTTTAGAAATGGAGATC TTACTGAAGGCACAGCCTATACAAACGCTGTTGGATTTATGCCTAACCTATCAGCTTATCCAAAATCTCACGGTAAA ACTGCCAAAAGTAACATTGTCAGTCAAGTTTACTTAAACGGAGACAAAACTAAACCTGTAACACTAACCATTACACT AAACGGTACACAGGAAACAGGAGACACAACTCCAAGTGCATACTCTATGTCATTTTCATGGGACTGGTCTGGCCACA ACTACATTAATGAAATATTTGCCACATCCTCTTACACTTTTTCATACATTGCCCAAGAATAAAGAATCGTTTGTGTT ATGTTTCAACGTGTTTATTTTTCAATTGCAGAAAATTTCAAGTCATTTTTCATTCAGTAGTATAGCCCCACCACCAC ATAGCTTATACAGATCACCGTACCTTAATCAAACTCACAGAACCCTAGTATTCAACCTGCCACCTCCCTCCCAACAC ACAGAGTACACAGTCCTTTCTCCCCGGCTGGCCTTAAAAAGCATCATATCATGGGTAACAGACATATTCTTAGGTGT TATATTCCACACGGTTTCCTGTCGAGCCAAACGCTCATCAGTGATATTAATAAACTCCCCGGGCAGCTCACTTAAGT TCATGTCGCTGTCCAGCTGCTGAGCCACAGGCTGCTGTCCAACTTGCGGTTGCTTAACGGGCGGCGAAGGAGAAGTC CACGCCTACATGGGGGTAGAGTCATAATCGTGCATCAGGATAGGGCGGTGGTGCTGCAGCAGCGCGCGAATAAACTG CTGCCGCCGCCGCTCCGTCCTGCAGGAATACAACATGGCAGTGGTCTCCTCAGCGATGATTCGCACCGCCCGCAGCA TAAGGCGCCTTGTCCTCCGGGCACAGCAGCGCACCCTGATCTCACTTAAATCAGCACAGTAACTGCAGCACAGCACC ACAATATTGTTCAAAATCCCACAGTGCAAGGCGCTGTATCCAAAGCTCATGGCGGGGACCACAGAACCCACGTGGCC ATCATACCACAAGCGCAGGTAGATTAAGTGGCGACCCCTCATAAACACGCTGGACATAAACATTACCTCTTTTGGCA TGTTGTAATTCACCACCTCCCGGTACCATATAAACCTCTGATTAAACATGGCGCCATCCACCACCATCCTAAACCAG CTGGCCAAAACCTGCCCGCCGGCTATACACTGCAGGGAACCGGGACTGGAACAATGACAGTGGAGAGCCCAGGACTC GTAACCATGGATCATCATGCTCGTCATGATATCAATGTTGGCACAACACAGGCACACGTGCATACACTTCCTCAGGA TTACAAGCTCCTCCCGCGTTAGAACCATATCCCAGGGAACAACCCATTCCTGAATCAGCGTAAATCCCACACTGCAG GGAAGACCTCGCACGTAACTCACGTTGTGCATTGTCAAAGTGTTACATTCGGGCAGCAGCGGATGATCCTCCAGTAT GGTAGCGCGGGTTTCTGTCTCAAAAGGAGGTAGACGATCCCTACTGTACGGAGTGCGCCGAGACAACCGAGATCGTG TTGGTCGTAGTGTCATGCCAAATGGAACGCCGGACGTAGTCATATTTCCTGAAGCAAAACCAGGTGCGGGCGTGACA AACAGATCTGCGTCTCCGGTCTCGCCGCTTAGATCGCTCTGTGTAGTAGTTGTAGTATATCCACTCTCTCAAAGCAT CCAGGCGCCCCCTGGCTTCGGGTTCTATGTAAACTCCTTCATGCGCCGCTGCCCTGATAACATCCACCACCGCAGAA TAAGCCACACCCAGCCAACCTACACATTCGTTCTGCGAGTCACACACGGGAGGAGCGGGAAGAGCTGGAAGAACCAT GTTTTTTTTTTTATTCCAAAAGATTATCCAAAACCTCAAAATGAAGATCTATTAAGTGAACGCGCTCCCCTCCGGTG GCGTGGTCAAACTCTACAGCCAAAGAACAGATAATGGCATTTGTAAGATGTTGCACAATGGCTTCCAAAAGGCAAAC GGCCCTCACGTCCAAGTGGACGTAAAGGCTAAACCCTTCAGGGTGAATCTCCTCTATAAACATTCCAGCACCTTCAA CCATGCCCAAATAATTCTCATCTCGCCACCTTCTCAATATATCTCTAAGCAAATCCCGAATATTAAGTCCGGCCATT GTAAAAATCTGCTCCAGAGCGCCCTCCACCTTCAGCCTCAAGCAGCGAATCATGATTGCAAAAATTCAGGTTCCTCA CAGACCTGTATAAGATTCAAAAGCGGAACATTAACAAAAATACCGCGATCCCGTAGGTCCCTTCGCAGGGCCAGCTG AACATAATCGTGCAGGTCTGCACGGACCAGCGCGGCCACTTCCCCGCCAGGAACCATGACAAAAGAACCCACACTGA TTATGACACGCATACTCGGAGCTATGCTAACCAGCGTAGCCCCGATGTAAGCTTGTTGCATGGGCGGCGATATAAAA TGCAAGGTGCTGCTCAAAAAATCAGGCAAAGCCTCGCGCAAAAAAGAAAGCACATCGTAGTCATGCTCATGCAGATA AAGGCAGGTAAGCTCCGGAACCACCACAGAAAAAGACACCATTTTTCTCTCAAACATGTCTGCGGGTTTCTGCATAA ACACAAAATAAAATAACAAAAAAACATTTAAACATTAGAAGCCTGTCTTACAACAGGAAAAACAACCCTTATAAGCA TAAGACGGACTACGGCCATGCCGGCGTGACCGTAAAAAAACTGGTCACCGTGATTAAAAAGCACCACCGACAGCTCC TCGGTCATGTCCGGAGTCATAATGTAAGACTCGGTAAACACATCAGGTTGATTCACATCGGTCAGTGCTAAAAAGCG ACCGAAATAGCCCGGGGGAATACATACCCGCAGGCGTAGAGACAACATTACAGCCCCCATAGGAGGTATAACAAAAT TAATAGGAGAGAAAAACACATAAACACCTGAAAAACCCTCCTGCCTAGGCAAAATAGCACCCTCCCGCTCCAGAACA ACATACAGCGCTTCCACAGCGGCAGCCATAACAGTCAGCCTTACCAGTAAAAAAGAAAACCTATTAAAAAAACACCA CTCGACACGGCACCAGCTCAATCAGTCACAGTGTAAAAAAGGGCCAAGTGCAGAGCGAGTATATATAGGACTAAAAA ATGACGTAACGGTTAAAGTCCACAAAAAACACCCAGAAAACCGCACGCGAACCTACGCCCAGAAACGAAAGCCAAAA AACCCACAACTTCCTCAAATCGTCACTTCCGTTTTCCCACGTTACGTAACTTCCCATTTTAAGAAAACTACAATTCC CAACACATACAAGTTACTCCGCCCTAAAACCTACGTCACCCGCCCCGTTCCCACGCCCCGCGCCACGTCACAAACTC CACCCCCTCATTATCATATTGGCTTCAATCCAAAATAAGGTATATTATTGATGATG

GenBank Accession No. AP_000226 MKRARPSEDTFNPVYPYDTETGPPTVPFLTPPFVSPNGFQESPPGVLSLRLSEPLVTSNGMLALKMGNGLSLDEAGN LTSQNVTTVSPPLKKTKSNINLEISAPLTVTSEALTVAAAAPLMVAGNTLTMQSQAPLTVHDSKLSIATQGPLTVSE GKLALQTSGPLTTTDSSTLTITASPPLTTATGSLGIDLKEPIYTONGKLGLKYGAPLHVTDDLNTLTVATGPGVTIN NTSLQTKVTGALGFDSQGNMOLNVAGGLRIDSQNRRLILDVSYPFDAQNQLNLRLGQGPLFINSAHNLDINYNKGLY LFTASNNSKKLEVNLSTAKGLMFDATAIAINAGDGLEFGSPNAPNTNPLKTKIGHGLEFDSNKAMVPKLGTGLSFDS TGAITVGNKNNDKLTLWTTPAPSPNCRLNAEKDAKLTLVLTKCGSQILATVSVLAVKGSLAPISGTVQSAHLIIRED ENGVLLNNSFLDPEYWNFRNGDLTEGTAYTNAVGEMPNLSAYPKSHGKTAKSNIVSQVYLNGDKTKPVTLTITLNGT QETGDTTPSAYSMSFSWDWSGHNYINEIFATSSYTFSYIAQE

GenBank Accession No. AP_000206 MRRAAMYEEGPPPSYESVVSAAPVAAALGSPFDAPLDPPFVPPRYLRPTGGRNSIRYSELAPLFDTTRVYLVDNKST DVASLNYONDHSNFLTTVIQNNDYSPGEASTQTINLDDRSHWGGDLKTILHTNMPNVNEFMFTNKEKARVMVSRLPT KDNQVELKYEWVEFTLPEGNYSETMTIDLMNNAIVEHYLKVGRONGVLESDIGVKFDTRNFRLGFDPVTGLVMPGVY TNEAFHPDIILLPGCGVDFTHSRLSNLLGIRKROPFQEGFRITYDDLEGGNIPALLDVDAYQASLKDDTEQGGGGAG GSNSSGSGAEENSNAAAAAMQPVEDMNDHAIRGDTFATRAEEKRAEAEAAAEAAAPAAQPEVEKPQKKPVIKPLTED SKKRSYNLISNDSTFTQYRSWYLAYNYGDPQTGIRSWTLLCTPDVTCGSEQVYWSLPDMMQDPVTFRSTRQISNFPV VGAELLPVHSKSFYNDQAVYSQLIROFTSLTHVFNRFPENQILARPPAPTITTVSENVPALTDHGTLPLRNSIGGVQ RVTITDARRRTCPYVYKALGIVSPRVLSSRTF

GenBank Accession No. AP_000211 MATPSMMPQWSYMHISGQDASEYLSPGLVQFARATETYFSLNNKERNPTVAPTHDVTTDRSQRLTLRFIPVDREDTA YSYKARFTLAVGDNRVLDMASTYFDIRGVLDRGPTFKPYSGTAYNALAPKGAPNPCEWDEAATALEINLEEEDDDNE DEVDEQAEQQKTHVFGQAPYSGINITKEGIQIGVEGOTPKYADKTFQPEPQIGESQWYETEINHAAGRVLKKTTPMK PCYGSYAKPTNENGGQGILVKOONGKLESQVEMQFFSTTEATAGNGDNLTPKVVLYSEDVDIETPDTHISYMPTIKE GNSRELMGQQSMPNRPNYIAFRDNFIGLMYYNSTGNMGVLAGQASQLNAVVDLQDRNTELSYQLLLDSIGDRTRYFS MWNQAVDSYDPDVRIIENHGTEDELPNYCFPLGGVINTETLTKVKPKTGQENGWEKDATEFSDKNEIRVGNNFAMEI NLNANLWRNFLYSNIALYLPDKLKYSPSNVKISDNPNTYDYMNKRVVAPGLVDCYINLGARWSLDYMDNVNPFNHHR NAGLRYRSMLLGNGRYVPFHIQVPQKFFAIKNLLLLPGSYTYEWNFRKDVNMVLQSSLGNDLRVDGASIKEDSICLY ATFFPMAHNTASTLEAMLRNDTNDOSFNDYLSAANMLYPIPANATNVPISIPSRNWAAFRGWAFTRLKTKETPSLGS GYDPYYTYSGSIPYLDGTFYLNHTFKKVAITFDSSVSWPGNDRLLTPNEFEIKRSVDGEGYNVAQCNMTKDWFLVQM LANYNIGYQGFYIPESYKDRMYSFFRNFQPMSRQVVDDTKYKDYQQVGILHQHNNSGFVGYLAPTMREGQAYPANFP YPLIGKTAVDSITQKKFLCDRTLWRIPFSSNFMSMGALTDLGQNLLYANSAHALDMTFEVDPMDEPTLLYVLFEVED VVRVHRPHRGVIETVYLRTPFSAGNATT

GenBank Accession No. AY128640 (SEQ ID NO: 216) CATCATCAATAATATACCTTATAGATGGAATGGTGCCAATATGTAAATGAGGTGATTTTAAAAAGTGTGGGCCGTGT GGTGATTGGCTGTGGGGTTAACGGTTAAAAGGGGCGGCGCGGCCGTGGGAAAATGACGTTTTATGGGGGTGGAGTTT TTTTGCAAGTTGTCGCGGGAAATGTTACGCATAAAAAGGCTTCTTTTCTCACGGAACTACTTAGTTTTCCCACGGTA TTTAACAGGAAATGAGGTAGTTTTGACCGGATGCAAGTGAAAATTGCTGATTTTCGCGCGAAAACTGAATGAGGAAG TGTTTTTCTGAATAATGTGGTATTTATGGCAGGGTGGAGTATTTGTTCAGGGCCAGGTAGACTTTGACCCATTACGT GGAGGTTTCGATTACCGTGTTTTTTACCTGAATTTCCGCGTACCGTGTCAAAGTCTTCTGTTTTTACGTAGGTGTCA GCTGATCGCTAGGGTATTTATACCTCAGGGTTTGTGTCAAGAGGCCACTCTTGAGTGCCAGCGAGAAGAGTTTTCTC CTCTGCGCCGGCAGTTTAATAATAAAAAAATGAGAGATTTGCGATTTCTGCCTCAGGAAATAATCTCTGCTGAGACT GGAAATGAAATATTGGAGCTTGTGGTGCACGCCCTGATGGGAGACGATCCGGAGCCACCTGTGCAGCTTTTTGAGCC TCCTACGCTTCAGGAACTGTATGATTTAGAGGTAGAGGGATCGGAGGATTCTAATGAGGAAGCTGTGAATGGCTTTT TTACCGATTCTATGCTTTTAGCTGCTAATGAAGGATTAGAATTAGATCCGCCTTTGGACACTTTCAATACTCCAGGG GTGATTGTGGAAAGCGGTACAGGTGTAAGAAAATTACCTGATTTGAGTTCCGTGGACTGTGATTTGCACTGCTATGA AGACGGGTTTCCTCCGAGTGATGAGGAGGACCATGAAAAGGAGCAGTCCATGCAGACTGCAGCGGGTGAGGGAGTGA AGGCTGCCAATGTTGGTTTTCAGTTGGATTGCCCGGAGCTTCCTGGACATGGCTGTAAGTCTTGTGAATTTCACAGG AAAAATACTGGAGTAAAGGAACTGTTATGTTCGCTTTGTTATATGAGAACGCACTGCCACTTTATTTACAGTAAGTG TGTTTAAGTTAAAATTTAAAGGAATATGCTGTTTTTCACATGTATATTGAGTGTGAGTTTTGTGCTTCTTATTATAG GTCCTGTGTCTGATGCTGATGAATCACCATCTCCTGATTCTACTACCTCACCTCCTGATATTCAAGCACCTGTTCCT GTGGACGTGCGCAAGCCCATTCCTGTGAAGCTTAAGCCTGGGAAACGTCCAGCAGTGGAGAAACTTGAGGACTTGTT ACAGGGTGGGGACGGACCTTTGGACTTGAGTACACGGAAACGTCCAAGACAATAAGTGTTCCATATCCGTGTTTACT TAAGGTGACGTCAATATTTGTGTGAGAGTGCAATGTAATAAAAATATGTTAACTGTTCACTGGTTTTTATTGCTTTT TGGGCGGGGACTCAGGTATATAAGTAGAAGCAGACCTGTGTGGTTAGCTCATAGGAGCTGGCTTTCATCCATGGAGG TTTGGGCCATTTTGGAAGACCTTAGGAAGACTAGGCAACTGTTAGAGAGCGCTTCGGACGGAGTCTCCGGTTTTTGG AGATTCTGGTTCGCTAGTGAATTAGCTAGGGTAGTTTTTAGGATAAAACAGGACTATAAACAAGAATTTGAAAAGTT GTTGGTAGATTGCCCAGGACTTTTTGAAGCTCTTAATTTGGGCCATCAGGTTCACTTTAAAGAAAAAGTTTTATCAG TTTTAGACTTTTCAACCCCAGGTAGAACTGCTGCTGCTGTGGCTTTTCTTACTTTTATATTAGATAAATGGATCCCG CAGACTCATTTCAGCAGGGGATACGTTTTGGATTTCATAGCCACAGCATTGTGGAGAACATGGAAGGTTCGCAAGAT GAGGACAATCTTAGGTTACTGGCCAGTGCAGCCTTTGGGTGTAGCGGGAATCCTGAGGCATCCACCGGTCATGCCAG CGGTTCTGGAGGAGGAACAGCAAGAGGACAACCCGAGAGCCGGCCTGGACCCTCCAGTGGAGGAGGCGGAGTAGCTG ACTTGTCTCCTGAACTGCAACGGGTGCTTACTGGATCTACGTCCACTGGACGGGATAGGGGCGTTAAGAGGGAGAGG GCATCCAGTGGTACTGATGCTAGATCTGAGTTGGCTTTAAGTTTAATGAGTCGCAGACGTCCTGAAACCATTTGGTG GCATGAGGTTCAGAAAGAGGGAAGGGATGAAGTTTCTGTATTGCAGGAGAAATATTCACTGGAACAGGTGAAAACAT GTTGGTTGGAGCCAGAGGATGATTGGGAGGTGGCCATTAAAAATTATGCCAAGATAGCTTTGAGGCCTGATAAACAG TATAAGATCAGTAGACGGATTAATATCCGGAATGCTTGTTACATATCTGGAAATGGGGCTGAGGTGGTAATAGATAC TCAAGACAAGACAGTTATTAGATGCTGCATGATGGATATGTGGCCTGGAGTAGTCGGTATGGAAGCAGTCACTTTTG TAAATGTTAAGTTTAGGGGAGATGGTTATAATGGAATAGTGTTTATGGCCAATACCAAACTTATATTGCATGGTTGT AGCTTTTTTGGTTTCAACAATACCTGTGTAGATGCCTGGGGACAGGTTAGTGTACGGGGGTGTAGTTTCTATGCGTG TTGGATTGCCACAGCTGGCAGAACCAAGAGTCAATTGTCTCTGAAGAAATGCATATTCCAAAGATGTAACCTGGGCA TTCTGAATGAAGGCGAAGCAAGGGTCCGTCACTGCGCTTCTACAGATACTGGATGTTTTATTTTAATTAAGGGAAAT GCCAGCGTAAAGCATAACATGATTTGTGGTGCTTCCGATGAGAGGCCTTATCAAATGCTCACTTGTGCTGGTGGGCA TTGTAATATGCTGGCTACTGTGCATATTGTTTCCCATCAACGCAAAAAATGGCCTGTTTTTGATCACAATGTGTTGA CCAAGTGCACCATGCATGCAGGTGGGCGTAGAGGAATGTTTATGCCTTACCAGTGTAACATGAATCATGTGAAAGTG TTGTTGGAACCAGATGCCTTTTCCAGAATGAGCCTAACAGGAATCTTTGACATGAACACGCAAATCTGGAAGATCCT GAGGTATGATGATACGAGATCGAGGGTGCGCGCATGCGAATGCGGAGGCAAGCATGCCAGGTTCCAGCCGGTGTGTG TAGATGTGACCGAAGATCTCAGACCGGATCATTTGGTTATTGCCCGCACTGGAGCAGAGTTCGGATCCAGTGGAGAA GAAACTGACTAAGGTGAGTATTGGGAAAACTTTGGGGTGGGATTTTCAGATGGACAGATTGAGTAAAAATTTGTTTT TTCTGTCTTGCAGCTGACATGAGTGGAAATGCTTCTTTTAAGGGGGGAGTCTTCAGCCCTTATCTGACAGGGCGTCT CCCATCCTGGGCAGGAGTTCGTCAGAATGTTATGGGATCTACTGTGGATGGAAGACCCGTTCAACCCGCCAATTCTT CAACGCTGACCTATGCTACTTTAAGTTCTTCACCTTTGGACGCAGCTGCAGCCGCTGCCGCCGCCTCTGTCGCCGCT AACACTGTGCTTGGAATGGGTTACTATGGAAGCATCGTGGCTAATTCCACTTCCTCTAATAACCCTTCTACACTGAC TCAGGACAAGTTACTTGTCCTTTTGGCCCAGCTGGAGGCTTTGACCCAACGTCTGGGTGAACTTTCTCAGCAGGTGG CCGAGTTGCGAGTACAAACTGAGTCTGCTGTCGGCACGGCAAAGTCTAAATAAAAAAAATTCCAGAATCAATGAATA AATAAACGAGCTTGTTGTTGATTTAAAATCAAGTGTTTTTATTTCATTTTTCGCGCACGGTATGCCCTGGACCACCG ATCTCGATCATTGAGAACTCGGTGGATTTTTTCCAGAATCCTATAGAGGTGGGATTGAATGTTTAGATACATGGGCA TTAGGCCGTCTTTGGGGTGGAGATAGCTCCATTGAAGGGATTCATGCTCCGGGGTAGTGTTGTAAATCACCCAGTCA TAACAAGGTCGCAGTGCATGGTGTTGCACAATATCTTTTAGAAGTAGGCTGATTGCCACAGATAAGCCCTTGGTGTA GGTGTTTACAAACCGGTTGAGCTGGGAGGGGTGCATTCGAGGTGAAATTATGTGCATTTTGGATTGGATTTTTAAGT TGGCAATATTGCCGCCAAGATCCCGTCTTGGGTTCATGTTATGAAGGACTACCAAGACGGTGTATCCGGTACATTTA GGAAATTTATCGTGCAGCTTGGATGGAAAAGCGTGGAAAAATTTGGAGACACCCTTGTGTCCTCCGAGATTTTCCAT GCACTCATCCATGATAATAGCAATGGGGCCGTGGGCAGCGGCGCGGGCAAACACGTTCCGTGGGTCTGACACATCAT AGTTATGTTCCTGAGTTAAATCATCATAAGCCATTTTAATGAATTTGGGGCGGAGCGTACCAGATTGGGGTATGAAT GTTCCTTCGGGCCCCGGAGCATAGTTCCCCTCACAGATTTGCATTTCCCAAGCTTTCAGTTCTGAGGGTGGAATCAT GTCCACCTGGGGGGCTATGAAGAACACCGTTTCGGGGGCGGGGGTGATTAGTTGGGATGATAGCAAGTTTCTGAGCA ATTGAGATTTGCCACATCCGGTGGGGCCATAAATAATTCCGATTACAGGTTGCAGGTGGTAGTTTAGGGAACGGCAA CTGCCGTCTTCTCGAAGCAAGGGGGCCACCTCGTTCATCATTTCCCTTACATGCATATTTTCCCGCACCAAATCCAT TAGGAGGCGCTCTCCTCCTAGTGATAGAAGTTCTTGTAGTGAGGAAAAGTTTTTCAGCGGTTTTAGACCGTCAGCCA TGGGCATTTTGGAAAGAGTTTGCTGCAAAAGTTCTAGTCTGTTCCACAGTTCAGTGATGTGTTCTATGGCATCTCGA TCCAGCAGACCTCCTCGTTTCGCGGGTTTGGACGGCTCCTGGAGTAGGGTATGAGACGATGGGCGTCCAGCGCTGCC AGGGTTCGGTCCTTCCAGGGTCTCAGTGTTCGAGTCAGGGTTGTTTCCGTCACAGTGAAGGGGTGTGCGCCTGCTTG GGCGCTTGCCAGGGTGCGCTTCAGACTCATTCTGCTGGTGGAGAACTTCTGTCGCTTGGCGCCCTGTATGTCGGCCA AGTAGCAGTTTACCATGAGTTCGTAGTTGAGCGCCTCGGCTGCGTGGCCTTTGGCGCGGAGCTTACCTTTGGAAGTT TTCTTGCATACCGGGCAGTATAGGCATTTCAGCGCATACAGCTTGGGCGCAAGGAAAATGGATTCTGGGGAGTATGC ATCCGCGCCGCAGGAGGCGCAAACAGTTTCACATTCCACCAGCCAGGTTAAATCCGGTTCATTGGGGTCAAAAACAA GTTTTCCGCCATATTTTTTGATGCGTTTCTTACCTTTGGTCTCCATAAGTTCGTGTCCTCGTTGAGTGACAAACAGG CTGTCCGTATCTCCGTAGACTGATTTTACAGGCCTCTTCTCCAGTGGAGTGCCTCGGTCTTCTTCGTACAGGAACTC TGACCACTCTGATACAAAGGCGCGCGTCCAGGCCAGCACAAAGGAGGCTATGTGGGAGGGGTAGCGATCGTTGTCAA CCAGGGGGTCCACCTTTTCCAAAGTATGCAAACACATGTCACCCTCTTCAACATCCAGGAATGTGATTGGCTTGTAG GTGTATTTCACGTGACCTGGGGTCCCCGCTGGGGGGGTATAAAAGGGGGGGGTTCTTTGCTCTTCCTCACTGTCTTC CGGATCGCTGTCCAGGAACGTCAGCTGTTGGGGTAGGTATTCCCTCTCGAAGGCGGGCATGACCTCTGCACTCAGGT TGTCAGTTTCTAAGAACGAGGAGGATTTGATATTGACAGTGCCGGTTGAGATGCCTTTCATGAGGTTTTCGTCCATT TGGTCAGAAAACACAATTTTTTTATTGTCAAGTTTGGTGGCAAATGATCCATACAGGGCGTTGGATAAAAGTTTGGC AATGGATCGCATGGTTTGGTTCTTTTCCTTGTCCGCGCGCTCTTTGGCGGCGATGTTGAGTTGGACATACTCGCGTG CCAGGCACTTCCATTCGGGGAAGATAGTTGTTAATTCATCTGGCACGATTCTCACTTGCCACCCTCGATTATGCAAG GTAATTAAATCCACACTGGTGGCCACCTCGCCTCGAAGGGGTTCATTGGTCCAACAGAGCCTACCTCCTTTCCTAGA ACAGAAAGGGGGAAGTGGGTCTAGCATAAGTTCATCGGGAGGGTCTGCATCCATGGTAAAGATTCCCGGAAGTAAAT CCTTATCAAAATAGCTGATGGGAGTGGGGTCATCTAAGGCCATTTGCCATTCTCGAGCTGCCAGTGCGCGCTCATAT GGGTTAAGGGGACTGCCCCAGGGCATGGGATGGGTGAGAGCAGAGGCATACATGCCACAGATGTCATAGACGTAGAT GGGATCCTCAAAGATGCCTATGTAGGTTGGATAGCATCGCCCCCCTCTGATACTTGCTCGCACATAGTCATATAGTT CATGTGATGGCGCTAGCAGCCCCGGACCCAAGTTGGTGCGATTGGGTTTTTCTGTTCTGTAGACGATCTGGCGAAAG ATGGCGTGAGAATTGGAAGAGATGGTGGGTCTTTGAAAAATGTTGAAATGGGCATGAGGTAGACCTACAGAGTCTCT GACAAAGTGGGCATAAGATTCTTGAAGCTTGGTTACCAGTTCGGCGGTGACAAGTACGTCTAGGGCGCAGTAGTCAA GTGTTTCTTGAATGATGTCATAACCTGGTTGGTTTTTCTTTTCCCACAGTTCGCGGTTGAGAAGGTATTCTTCGCGA TCCTTCCAGTACTCTTCTAGCGGAAACCCGTCTTTGTCTGCACGGTAAGATCCTAGCATGTAGAACTGATTAACTGC CTTGTAAGGGCAGCAGCCCTTCTCTACGGGTAGAGAGTATGCTTGAGCAGCTTTTCGTAGCGAAGCGTGAGTAAGGG CAAAGGTGTCTCTGACCATGACTTTGAGAAATTGGTATTTGAAGTCCATGTCGTCACAGGCTCCCTGTTCCCAGAGT TGGAAGTCTACCCGTTTCTTGTAGGCGGGGTTGGGCAAAGCGAAAGTAACATCATTGAAGAGAATCTTACCGGCTCT GGGCATAAAATTGCGAGTGATGCGGAAAGGCTGTGGTACTTCCGCTCGATTGTTGATCACCTGGGCAGCTAGGACGA TTTCGTCGAAACCGTTGATGTTGTGTCCTACGATGTATAATTCTATGAAACGCGGCGTGCCTCTGACGTGAGGTAGC TTACTGAGCTCATCAAAGGTTAGGTCTGTGGGGTCAGATAAGGCGTAGTGTTCGAGAGCCCATTCGTGCAGGTGAGG ATTTGCATGTAGGAATGATGACCAAAGATCTACCGCCAGTGCTGTTTGTAACTGGTCCCGATACTGACGAAAATGCC GGCCAATTGCCATTTTTTCTGGAGTGACACAGTAGAAGGTTCTGGGGTCTTGTTGCCATCGATCCCACTTGAGTTTA ATGGCTAGATCGTGGGCCATGTTGACGAGACGCTCTTCTCCTGAGAGTTTCATGACCAGCATGAAAGGAACTAGTTG TTTGCCAAAGGATCCCATCCAGGTGTAAGTTTCCACATCGTAGGTCAGGAAGAGTCTTTCTGTGCGAGGATGAGAGC CGATCGGGAAGAACTGGATTTCCTGCCACCAGTTGGAGGATTGGCTGTTGATGTGATGGAAGTAGAAGTTTCTGCGG CGCGCCGAGCATTCGTGTTTGTGCTTGTACAGACGGCCGCAGTAGTCGCAGCGTTGCACGGGTTGTATCTCGTGAAT GAGCTGTACCTGGCTTCCCTTGACGAGAAATTTCAGTGGGAAGCCGAGGCCTGGCGATTGTATCTCGTGCTCTTCTA TATTCGCTGTATCGGCCTGTTCATCTTCTGTTTCGATGGTGGTCATGCTGACGAGCCCCCGCGGGAGGCAAGTCCAG ACCTCGGCGCGGGAGGGGCGGAGCTGAAGGACGAGAGCGCGCAGGCTGGAGCTGTCCAGAGTCCTGAGACGCTGCGG ACTCAGGTTAGTAGGTAGGGACAGAAGATTAACTTGCATGATCTTTTCCAGGGCGTGCGGGAGGTTCAGATGGTACT TGATTTCCACAGGTTCGTTTGTAGAGACGTCAATGGCTTGCAGGGTTCCGTGTCCTTTGGGCGCCACTACCGTACCT TTGTTTTTTCTTTTGATCGGTGGTGGCTCTCTTGCTTCTTGCATGCTCAGAAGCGGTGACGGGGACGCGCGCCGGGC GGCAGCGGTTGTTCCGGACCCGGGGGCATGGCTGGTAGTGGCACGTCGGCGCCGCGCACGGGCAGGTTCTGGTATTG CGCTCTGAGAAGACTTGCGTGCGCCACCACGCGTCGATTGACGTCTTGTATCTGACGTCTCTGGGTGAAAGCTACCG GCCCCGTGAGCTTGAACCTGAAAGAGAGTTCAACAGAATCAATTTCGGTATCGTTAACGGCAGCTTGTCTCAGTATT TCTTGTACGTCACCAGAGTTGTCCTGGTAGGCGATCTCCGCCATGAACTGCTCGATTTCTTCCTCCTGAAGATCTCC GCGACCCGCTCTTTCGACGGTGGCCGCGAGGTCATTGGAGATACGGCCCATGAGTTGGGAGAATGCATTCATGCCCG CCTCGTTCCAGACGCGGCTGTAAACCACGGCCCCCTCGGAGTCTCTTGCGCGCATCACCACCTGAGCGAGGTTAAGC TCCACGTGTCTGGTTAAGACCGCATAGTTGCATAGGCGCTGAAAAAGGTAGTTGAGTGTGGTGGCAATGTGTTCGGC GACGAAGAAATACATGATCCATCGTCTCAGCGGCATTTCGCTAACATCGCCCAGAGCTTCCAAGCGCTCCATGGCCT CGTAGAAGTCCACGGCAAAATTAAAAAACTGGGAGTTTCGCGCGGACACGGTCAATTCCTCCTCGAGAAGACGGATG AGTTCGGCTATGGTGGCCCGTACTTCGCGTTCGAAGGCTCCCGGGATCTCTTCTTCCTCTTCTATCTCTTCTTCCAC TAACATCTCTTCTTCGTCTTCAGGCGGGGGCGGAGGGGGCACGCGGCGACGTCGACGGCGCACGGGCAAACGGTCGA TGAATCGTTCAATGACCTCTCCGCGGCGGCGGCGCATGGTTTCAGTGACGGCGCGGCCGTTCTCGCGCGGTCGCAGA GTAAAAACACCGCCGCGCATCTCCTTAAAGTGGTGACTGGGAGGTTCTCCGTTTGGGAGGGAGAGGGCGCTGATTAT CGACGAAAGCGTCTAACCAGTCACAGTCACAAGGTAGGCTGAGTACGGCTTCTTGTGGGCGGGGGTGGTTATGTGTT CGGTCTGGGTCTTCTGTTTCTTCTTCATCTCGGGAAGGTGAGACGATGCTGCTGGTGATGAAATTAAAGTAGGCAGT TCTAAGACGGCGGATGGTGGCGAGGAGCACCAGGTCTTTGGGTCCGGCTTGCTGGATACGCAGGCGATTGGCCATTC CCCAAGCATTATCCTGACATCTAGCAAGATCTTTGTAGTAGTCTTGCATGAGCCGTTCTACGGGCACTTCTTCCTCA CCCGTTCTGCCATGCATACGTGTGAGTCCAAATCCGCGCATTGGTTGTACCAGTGCCAAGTCAGCTACGACTCTTTC GGCGAGGATGGCTTGCTGTACTTGGGTAAGGGTGGCTTGAAAGTCATCAAAATCCACAAAGCGGTGGTAAGCCCCTG TATTAATGGTGTAAGCACAGTTGGCCATGACTGACCAGTTAACTGTCTGGTGACCAGGGCGCACGAGCTCGGTGTAT TTAAGGCGCGAATAGGCGCGGGTGTCAAAGATGTAATCGTTGCAGGTGCGCACCAGATACTGGTACCCTATAAGAAA ATGCGGCGGTGGTTGGCGGTAGAGAGGCCATCGTTCTGTAGCTGGAGCGCCAGGGGCGAGGTCTTCCAACATAAGGC GGTGATAGCCGTAGATGTACCTGGACATCCAGGTGATTCCTGCGGCGGTAGTAGAAGCCCGAGGAAACTCGCGTACG CGGTTCCAAATGTTGCGTAGCGGCATGAAGTAGTTCATTGTAGGCACGGTTTGACCAGTGAGGCGCGCGCAGTCATT GATGCTCTATAGACACGGAGAAAATGAAAGCGTTCAGCGACTCGACTCCGTAGCCTGGAGGAACGTGAACGGGTTGG GTCGCGGTGTACCCCGGTTCGAGACTTGTACTCGAGCCGGCCGGAGCCGCGGCTAACGTGGTATTGGCACTCCCGTC TCGACCCAGCCTACAAAAATCCAGGATACGGAATCGAGTCGTTTTGCTGGTTTCCGAATGGCAGGGAAGTGAGTCCT ATTTTTTTTTTTTTTTGCCGCTCAGAATGCATCCCGTGCTGCGACAGATGCGCCCCCAACAACAGCCCCCCTCGCAG CAGCAGCAGCAGCAACCACAAAAGGCTGTCCCTGCAACTACTGCAACTGCCGCCGTGAGCGGTGCGGGACAGCCCGC CTATGATCTGGACTTGGAAGAGGGCGAAGGACTGGCACGTCTAGGTGCGCCTTCGCCCGAGCGGCATCCGCGAGTTC AACTGAAAAAAGATTCTCGCGAGGCGTATGTGCCCCAACAGAACCTATTTAGAGACAGAAGCGGCGAGGAGCCGGAG GAGATGCGAGCTTCCCGCTTTAACGCGGGTCGTGAGCTGCGTCACGGTTTGGACCGAAGACGAGTGTTGCGAGACGA GGATTTCGAAGTTGATGAAGTGACAGGGATCAGTCCTGCCAGGGCACACGTGGCTGCAGCCAACCTTGTATCGGCTT ACGAGCAGACAGTAAAGGAAGAGCGTAACTTCCAAAAGTCTTTTAATAATCATGTGCGAACCCTGATTGCCCGCGAA GAAGTTACCCTTGGTTTGATGCATTTGTGGGATTTGATGGAAGCTATCATTCAGAACCCTACTAGCAAACCTCTGAC CGCCCAGCTGTTTCTGGTGGTGCAACACAGCAGAGACAATGAGGCTTTCAGAGAGGCGCTGCTGAACATCACCGAAC CCGAGGGGAGATGGTTGTATGATCTTATCAACATTCTACAGAGTATCATAGTGCAGGAGCGGAGCCTGGGCCTGGCC GAGAAGGTAGCTGCCATCAATTACTCGGTTTTGAGCTTGGGAAAATATTACGCTCGCAAAATCTACAAGACTCCATA CGTTCCCATAGACAAGGAGGTGAAGATAGATGGGTTCTACATGCGCATGACGCTCAAGGTCTTGACCCTGAGCGATG ATCTTGGGGTGTATCGCAATGACAGAATGCATCGCGCGGTTAGCGCCAGCAGGAGGCGCGAGTTAAGCGACAGGGAA CTGATGCACAGTTTGCAAAGAGCTCTGACTGGAGCTGGAACCGAGGGTGAGAATTACTTCGACATGGGAGCTGACTT GCAGTGGCAGCCTAATCGCAGGGCTCTGAGCGCCGCGACGGCAGGATGTGAGCTTCCTTACATAGAAGAGGCGGATG AAGGCGAGGAGGAAGAGGGCGAGTACTTGGAAGACTGATGGCACAACCCGTGTTTTTTGCTAGATGGAACAGCAAGC ACCGGATCCCGCAATGCGGGCGGCGCTGCAGAGCCAGCCGTCCGGCATTAACTCCTCGGACGATTGGACCCAGGCCA TGCAACGTATCATGGCGTTGACGACTCGCAACCCCGAAGCCTTTAGACAGCAACCCCAGGCCAACCGTCTATCGGCC ATCATGGAAGCTGTAGTGCCTTCCCGATCTAATCCCACTCATGAGAAGGTCCTGGCCATCGTGAACGCGTTGGTGGA GAACAAAGCTATTCGTCCAGATGAGGCCGGACTGGTATACAACGCTCTCTTAGAACGCGTGGCTCGCTACAACAGTA GCAATGTGCAAACCAATTTGGACCGTATGATAACAGATGTACGCGAAGCCGTGTCTCAGCGCGAAAGGTTCCAGCGT GATGCCAACCTGGGTTCGCTGGTGGCGTTAAATGCTTTCTTGAGTACTCAGCCTGCTAATGTGCCGCGTGGTCAACA GGATTATACTAACTTTTTAAGTGCTTTGAGACTGATGGTATCAGAAGTACCTCAGAGCGAAGTGTATCAGTCCGGTC CTGATTACTTCTTTCAGACTAGCAGACAGGGCTTGCAGACGGTAAATCTGAGCCAAGCTTTTAAAAACCTTAAAGGT TTGTGGGGAGTGCATGCCCCGGTAGGAGAAAGAGCAACCGTGTCTAGCTTGTTAACTCCGAACTCCCGCCTGTTATT ACTGTTGGTAGCTCCTTTCACCGACAGCGGTAGCATCGACCGTAATTCCTATTTGGGTTACCTACTAAACCTGTATC GCGAAGCCATAGGGCAAAGTCAGGTGGACGAGCAGACCTATCAAGAAATTACCCAAGTCAGTCGCGCTTTGGGACAG GAAGACACTGGCAGTTTGGAAGCCACTCTGAACTTCTTGCTTACCAATCGGTCTCAAAAGATCCCTCCTCAATATGC TCTTACTGCGGAGGAGGAGAGGATCCTTAGATATGTGCAGCAGAGCGTGGGATTGTTTCTGATGCAAGAGGGGGCAA CTCCGACTGCAGCACTGGACATGACAGCGCGAAATATGGAGCCCAGCATGTATGCCAGTAACCGACCTTTCATTAAC AAACTGCTGGACTACTTGCACAGAGCTGCCGCTATGAACTCTGATTATTTCACCAATGCCATCTTAAACCCGCACTG GCTGCCCCCACCTGGTTTCTACACGGGCGAATATGACATGCCCGACCCTAATGACGGATTTCTGTGGGACGACGTGG ACAGCGATGTTTTTTCACCTCTTTCTGATCATCGCACGTGGAAAAAGGAAGGCGGTGATAGAATGCATTCTTCTGCA TCGCTGTCCGGGGTCATGGGTGCTACCGCGGCTGAGCCCGAGTCTGCAAGTCCTTTTCCTAGTCTACCCTTTTCTCT ACACAGTGTACGTAGCAGCGAAGTGGGTAGAATAAGTCGCCCGAGTTTAATGGGCGAAGAGGAGTACCTAAACGATT CCTTGCTCAGACCGGCAAGAGAAAAAAATTTCCCAAACAATGGAATAGAAAGTTTGGTGGATAAAATGAGTAGATGG AAGACTTATGCTCAGGATCACAGAGACGAGCCTGGGATCATGGGGACTACAAGTAGAGCGAGCCGTAGACGCCAGCG CCATGACAGACAGAGGGGTCTTGTGTGGGACGATGAGGATTCGGCCGATGATAGCAGCGTGTTGGACTTGGGTGGGA GAGGAAGGGGCAACCCGTTTGCTCATTTGCGCCCTCGCTTGGGTGGTATGTTGTGAAAAAAAATAAAAAAGAAAAAC TCACCAAGGCCATGGCGACGAGCGTACGTTCGTTCTTCTTTATTATCTGTGTCTAGTATAATGAGGCGAGTCGTGCT AGGCGGAGCGGTGGTGTATCCGGAGGGTCCTCCTCCTTCGTACGAGAGCGTGATGCAGCAGCAGCAGGCGACGGCGG TGATGCAATCCCCACTGGAGGCTCCCTTTGTGCCTCCGCGATACCTGGCACCTACGGAGGGCAGAAACAGCATTCGT TACTCGGAACTGGCACCTCAGTACGATACCACCAGGTTGTATCTGGTGGACAACAAGTCGGCGGACATTGCTTCTCT GAACTATCAGAATGACCACAGCAACTTCTTGACCACGGTGGTGCAGAACAATGACTTTACCCCTACGGAAGCCAGCA CCCAGACCATTAACTTTGATGAACGATCGCGGTGGGGCGGTCAGCTAAAGACCATCATGCATACTAACATGCCAAAC GTGAACGAGTATATGTTTAGTAACAAGTTCAAAGCGCGTGTGATGGTGTCCAGAAAACCTCCCGACGGTGCTGCAGT TGGGGATACTTATGATCACAAGCAGGATATTTTGGAATATGAGTGGTTCGAGTTTACTTTGCCAGAAGGCAACTTTT CAGTTACTATGACTATTGATTTGATGAACAATGCCATCATAGATAATTACTTGAAAGTGGGTAGACAGAATGGAGTG CTTGAAAGTGACATTGGTGTTAAGTTCGACACCAGGAACTTCAAGCTGGGATGGGATCCCGAAACCAAGTTGATCAT GCCTGGAGTGTATACGTATGAAGCCTTCCATCCTGACATTGTCTTACTGCCTGGCTGCGGAGTGGATTTTACCGAGA GTCGTTTGAGCAACCTTCTTGGTATCAGAAAAAAACAGCCATTTCAAGAGGGTTTTAAGATTTTGTATGAAGATTTA GAAGGTGGTAATATTCCGGCCCTCTTGGATGTAGATGCCTATGAGAACAGTAAGAAAGAACAAAAAGCCAAAATAGA AGCTGCTACAGCTGCTGCAGAAGCTAAGGCAAACATAGTTGCCAGCGACTCTACAAGGGTTGCTAACGCTGGAGAGG TCAGAGGAGACAATTTTGCGCCAACACCTGTTCCGACTGCAGAATCATTATTGGCCGATGTGTCTGAAGGAACGGAC GTGAAACTCACTATTCAACCTGTAGAAAAAGATAGTAAGAATAGAAGCTATAATGTGTTGGAAGACAAAATCAACAC AGCCTATCGCAGTTGGTATCTTTCGTACAATTATGGCGATCCCGAAAAAGGAGTGCGTTCCTGGACATTGCTCACCA CCTCAGATGTCACCTGCGGAGCAGAGCAGGTTTACTGGTCGCTTCCAGACATGATGAAGGATCCTGTCACTTTCCGC TCCACTAGACAAGTCAGTAACTACCCTGTGGTGGGTGCAGAGCTTATGCCCGTCTTCTCAAAGAGCTTCTACAACGA ACAAGCTGTGTACTCCCAGCAGCTCCGCCAGTCCACCTCGCTTACGCACGTCTTCAACCGCTTTCCTGAGAACCAGA TTTTAATCCGTCCGCCGGCGCCCACCATTACCACCGTCAGTGAAAACGTTCCTGCTCTCACAGATCACGGGACCCTG CCGTTGCGCAGCAGTATCCGGGGAGTCCAACGTGTGACCGTTACTGACGCCAGACGCCGCACCTGTCCCTACGTGTA CAAGGCACTGGGCATAGTCGCACCGCGCGTCCTTTCAAGCCGCACTTTCTAAAAAAAAAATGTCCATTCTTATCTCG CCCAGTAATAACACCGGTTGGGGTCTGCGCGCTCCAAGCAAGATGTACGGAGGCGCACGCAAACGTTCTACCCAACA TCCCGTGCGTGTTCGCGGACATTTTCGCGCTCCATGGGGTGCCCTCAAGGGCCGCACTCGCGTTCGAACCACCGTCG ATGATGTAATCGATCAGGTGGTTGCCGACGCCCGTAATTATACTCCTACTGCGCCTACATCTACTGTGGATGCAGTT ATTGACAGTGTAGTGGCTGACGCTCGCAACTATGCTCGACGTAAGAGCCGGCGAAGGCGCATTGCCAGACGCCACCG AGCTACCACTGCCATGCGAGCCGCAAGAGCTCTGCTACGAAGAGCTAGACGCGTGGGGCGAAGAGCCATGCTTAGGG CGGCCAGACGTGCAGCTTCGGGCGCCAGCGCCGGCAGGTCCCGCAGGCAAGCAGCCGCTGTCGCAGCGGCGACTATT GCCGACATGGCCCAATCGCGAAGAGGCAATGTATACTGGGTGCGTGACGCTGCCACCGGTCAACGTGTACCCGTGCG CACCCGTCCCCCTCGCACTTAGAAGATACTGAGCAGTCTCCGATGTTGTGTCCCAGCGGCGAGGATGTCCAAGCGCA AATACAAGGAAGAAATGCTGCAGGTTATCGCACCTGAAGTCTACGGCCAACCGTTGAAGGATGAAAAAAAACCCCGC AAAATCAAGCGGGTTAAAAAGGACAAAAAAGAAGAGGAAGATGGCGATGATGGGCTGGCGGAGTTTGTGCGCGAGTT TGCCCCACGGCGACGCGTGCAATGGCGTGGGCGCAAAGTTCGACATGTGTTGAGACCTGGAACTTCGGTGGTCTTTA CACCCGGCGAGCGTTCAAGCGCTACTTTTAAGCGTTCCTATGATGAGGTGTACGGGGATGATGATATTCTTGAGCAG GCGGCTGACCGATTAGGCGAGTTTGCTTATGGCAAGCGTAGTAGAATAACTTCCAAGGATGAGACAGTGTCAATACC CTTGGATCATGGAAATCCCACCCCTAGTCTTAAACCGGTCACTTTGCAGCAAGTGTTACCCGTAACTCCGCGAACAG GTGTTAAACGCGAAGGTGAAGATTTGTATCCCACTATGCAACTGATGGTACCCAAACGCCAGAAGTTGGAGGACGTT TTGGAGAAAGTAAAAGTGGATCCAGATATTCAACCTGAGGTTAAAGTGAGACCCATTAAGCAGGTAGCGCCTGGTCT GGGGGTACAAACTGTAGACATTAAGATTCCCACTGAAAGTATGGAAGTGCAAACTGAACCCGCAAAGCCTACTGCCA CCTCCACTGAAGTGCAAACGGATCCATGGATGCCCATGCCTATTACAACTGACGCCGCCGGTCCCACTCGAAGATCC CGACGAAAGTACGGTCCAGCAAGTCTGTTGATGCCCAATTATGTTGTACACCCATCTATTATTCCTACTCCTGGTTA CCGAGGCACTCGCTACTATCGCAGCCGAAACAGTACCTCCCGCCGTCGCCGCAAGACACCTGCAAATCGCAGTCGTC GCCGTAGACGCACAAGCAAACCGACTCCCGGCGCCCTGGTGCGGCAAGTGTACCGCAATGGTAGTGCGGAACCTTTG ACACTGCCGCGTGCGCGTTACCATCCGAGTATCATCACTTAATCAATGTTGCCGCTGCCTCCTTGCAGATATGGCCC TCACTTGTCGCCTTCGCGTTCCCATCACTGGTTACCGAGGAAGAAACTCGCGCCGTAGAAGAGGGATGTTGGGACGC GGAATGCGACGCTACAGGCGACGGCGTGCTATCCGCAAGCAATTGCGGGGTGGTTTTTTACCAGCCTTAATTCCAAT TATCGCTGCTGCAATTGGCGCGATACCAGGCATAGCTTCCGTGGCGGTTCAGGCCTCGCAACGACATTGACATTGGA AAAAAAACGTATAAATAAAAAAAAATACAATGGACTCTGACACTCCTGGTCCTGTGACTATGTTTTCTTAGAGATGG AAGACATCAATTTTTCATCCTTGGCTCCGCGACACGGCACGAAGCCGTACATGGGCACCTGGAGCGACATCGGCACG AGCCAACTGAACGGGGGCGCCTTCAATTGGAGCAGTATCTGGAGCGGGCTTAAAAATTTTGGCTCAACCATAAAAAC ATACGGGAACAAAGCTTGGAACAGCAGTACAGGACAGGCGCTTAGAAATAAACTTAAAGACCAGAACTTCCAACAAA AAGTAGTCGATGGGATAGCTTCCGGCATCAATGGAGTGGTAGATTTGGCTAACCAGGCTGTGCAGAAAAAGATAAAC AGTCGTTTGGACCCGCCGCCAGCAACCCCAGGTGAAATGCAAGTGGAGGAAGAAATTCCTCCGCCAGAAAAACGAGG CGACAAGCGTCCGCGTCCCGATTTGGAAGAGACGCTGGTGACGCGCGTAGATGAACCGCCTTCTTATGAGGAAGCAA CGAAGCTTGGAATGCCCACCACTAGACCGATAGCCCCAATGGCCACCGGGGTGATGAAACCTTCTCAGTTGCATCGA CCCGTCACCTTGGATTTGCCCCCTCCCCCTGCTGCTACTGCTGTACCCGCTTCTAAGCCTGTCGCTGCCCCGAAACC AGTCGCCGTAGCCAGGTCACGTCCCGGGGGCGCTCCTCGTCCAAATGCGCACTGGCAAAATACTCTGAACAGCATCG TGGGTCTAGGCGTGCAAAGTGTAAAACGCCGTCGCTGCTTTTAATTAAATATGGAGTAGCGCTTAACTTGCCTATCT GTGTATATGTGTCATTACACGCCGTCACAGCAGCAGAGGAAAAAAGGAAGAGGTCGTGCGTCGACGCTGAGTTACTT TCAAGATGGCCACCCCATCGATGCTGCCCCAATGGGCATACATGCACATCGCCGGACAGGATGCTTCGGAGTACCTG AGTCCGGGTCTGGTGCAGTTCGCCCGCGCCACAGACACCTACTTCAATCTGGGAAATAAGTTTAGAAATCCCACCGT AGCGCCGACCCACGATGTGACCACCGACCGTAGCCAGCGGCTCATGTTGCGCTTCGTGCCCGTTGACCGGGAGGACA ATACATACTCTTACAAAGTGCGGTACACCCTGGCCGTGGGCGACAACAGAGTGCTGGATATGGCCAGCACGTTCTTT GACATTAGGGGCGTGTTGGACAGAGGTCCCAGTTTCAAACCCTATTCTGGTACGGCTTACAACTCTCTGGCTCCTAA AGGCGCTCCAAATGCATCTCAATGGATTGCAAAAGGCGTACCAACTGCAGCAGCCGCAGGCAATGGTGAAGAAGAAC ATGAAACAGAGGAGAAAACTGCTACTTACACTTTTGCCAATGCTCCTGTAAAAGCCGAGGCTCAAATTACAAAAGAG GGCTTACCAATAGGTTTGGAGATTTCAGCTGAAAACGAATCTAAACCCATCTATGCAGATAAACTTTATCAGCCAGA ACCTCAAGTGGGAGATGAAACTTGGACTGACCTAGACGGAAAAACCGAAGAGTATGGAGGCAGGGCTCTAAAGCCTA CTACTAACATGAAACCCTGTTACGGGTCCTATGCGAAGCCTACTAATTTAAAAGGTGGTCAGGCAAAACCGAAAAAC TCGGAACCGTCGAGTGAAAAAATTGAATATGATATTGACATGGAATTTTTTGATAACTCATCGCAAAGAACAAACTT CAGTCCTAAAATTGTCATGTATGCAGAAAATGTAGGTTTGGAAACGCCAGACACTCATGTAGTGTACAAACCTGGAA CAGAAGACACAAGTTCCGAAGCTAATTTGGGACAACAGTCTATGCCCAACAGACCCAACTACATTGGCTTCAGAGAT AACTTTATTGGACTCATGTACTATAACAGTACTGGTAACATGGGGGTGCTGGCTGGTCAAGCGTCTCAGTTAAATGC AGTGGTTGACTTGCAGGACAGAAACACAGAACTTTCTTACCAACTCTTGCTTGACTCTCTGGGCGACAGAACCAGAT ACTTTAGCATGTGGAATCAGGCTGTGGACAGTTATGATCCTGATGTACGTGTTATTGAAAATCATGGTGTGGAAGAT GAACTTCCCAACTATTGTTTTCCACTGGACGGCATAGGTGTTCCAACAACCAGTTACAAATCAATAGTTCCAAATGG AGAAGATAATAATAATTGGAAAGAACCTGAAGTAAATGGAACAAGTGAGATCGGACAGGGTAATTTGTTTGCCATGG AAATTAACCTTCAAGCCAATCTATGGCGAAGTTTCCTTTATTCCAATGTGGCTCTGTATCTCCCAGACTCGTACAAA TACACCCCGTCCAATGTCACTCTTCCAGAAAACAAAAACACCTACGACTACATGAACGGGCGGGTGGTGCCGCCATC TCTAGTAGACACCTATGTGAACATTGGTGCCAGGTGGTCTCTGGATGCCATGGACAATGTCAACCCATTCAACCACC ACCGTAACGCTGGCTTGCGTTACCGATCTATGCTTCTGGGTAACGGACGTTATGTGCCTTTCCACATACAAGTGCCT CAAAAATTCTTCGCTGTTAAAAACCTGCTGCTTCTCCCAGGCTCCTACACTTATGAGTGGAACTTTAGGAAGGATGT GAACATGGTTCTACAGAGTTCCCTCGGTAACGACCTGCGGGTAGATGGCGCCAGCATCAGTTTCACGAGCATCAACC TCTATGCTACTTTTTTCCCCATGGCTCACAACACCGCTTCCACCCTTGAAGCCATGCTGCGGAATGACACCAATGAT CAGTCATTCAACGACTACCTATCTGCAGCTAACATGCTCTACCCCATTCCTGCCAATGCAACCAATATTCCCATTTC CATTCCTTCTCGCAACTGGGCGGCTTTCAGAGGCTGGTCATTTACCAGACTGAAAACCAAAGAAACTCCCTCTTTGG GGTCTGGATTTGACCCCTACTTTGTCTATTCTGGTTCTATTCCCTACCTGGATGGTACCTTCTACCTGAACCACACT TTTAAGAAGGTTTCCATCATGTTTGACTCTTCAGTGAGCTGGCCTGGAAATGACAGGTTACTATCTCCTAACGAATT TGAAATAAAGCGCACTGTGGATGGCGAAGGCTACAACGTAGCCCAATGCAACATGACCAAAGACTGGTTCTTGGTAC AGATGCTCGCCAACTACAACATCGGCTATCAGGGCTTCTACATTCCAGAAGGATACAAAGATCGCATGTATTCATTT TTCAGAAACTTCCAGCCCATGAGCAGGCAGGTGGTTGATGAGGTCAATTACAAAGACTTCAAGGCCGTCGCCATACC CTACCAACACAACAACTCTGGCTTTGTGGGTTACATGGCTCCGACCATGCGCCAAGGTCAACCCTATCCCGCTAACT ATCCCTATCCACTCATTGGAACAACTGCCGTAAATAGTGTTACGCAGAAAAAGTTCTTGTGTGACAGAACCATGTGG CGCATACCGTTCTCGAGCAACTTCATGTCTATGGGGGCCCTTACAGACTTGGGACAGAATATGCTCTATGCCAACTC AGCTCATGCTCTGGACATGACCTTTGAGGTGGATCCCATGGATGAGCCCACCCTGCTTTATCTTCTCTTCGAAGTTT TCGACGTGGTCAGAGTGCATCAGCCACACCGCGGCATCATCGAGGCAGTCTACCTGCGTACACCGTTCTCGGCCGGT AACGCTACCACGTAAGAAGCTTCTTGCTTCTTGCAAATAGCAGCTGCAACCATGGCCTGCGGATCCCAAAACGGCTC CAGCGAGCAAGAGCTCAGAGCCATTGTCCAAGACCTGGGTTGCGGACCCTATTTTTTGGGAACCTACGATAAGCGCT TCCCGGGGTTCATGGCCCCCGATAAGCTCGCCTGTGCCATTGTAAATACGGCCGGACGTGAGACGGGGGGAGAGCAC TGGTTGGCTTTCGGTTGGAACCCACGTTCTAACACCTGCTACCTTTTTGATCCTTTTGGATTCTCGGATGATCGTCT CAAACAGATTTACCAGTTTGAATATGAGGGTCTCCTGCGCCGCAGCGCTCTTGCTACCAAGGACCGCTGTATTACGC TGGAAAAATCTACCCAGACCGTGCAGGGCCCCCGTTCTGCCGCCTGCGGACTTTTCTGCTGCATGTTCCTTCACGCC TTTGTGCACTGGCCTGACCGTCCCATGGACGGAAACCCCACCATGAAATTGCTAACTGGAGTGCCAAACAACATGCT TCATTCTCCTAAAGTCCAGCCCACCCTGTGTGACAATCAAAAAGCACTCTACCATTTTCTTAATACCCATTCGCCTT ATTTTCGCTCTCATCGTACACACATCGAAAGGGCCACTGCGTTCGACCGTATGGATGTTCAATAATGACTCATGTAA ACAACGTGTTCAATAAACATCACTTTATTTTTTTACATGTATCAAGGCTCTGGATTACTTATTTATTTACAAGTCGA ATGGGTTCTGACGAGAATCAGAATGACCCGCAGGCAGTGATACGTTGCGGAACTGATACTTGGGTTGCCACTTGAAT TCGGGAATCACCAACTTGGGAACCGGTATATCGGGCAGGATGTCACTCCACAGCTTTCTGGTCAGCTGCAAAGCTCC AAGCAGGTCAGGAGCCGAAATCTTGAAATCACAATTAGGACCAGTGCTCTGAGCGCGAGAGTTGCGGTACACCGGAT TGCAGCACTGAAACACCATCAGCGACGGATGTCTCACGCTTGCCAGCACGGTGGGATCTGCAATCATGCCCACATCC AGATCTTCAGCATTGGCAATGCTGAACGGGGTCATCTTGCAGGTCTGCCTACCCATGGCGGGCACCCAATTAGGCTT GTGGTTGCAATCGCAGTGCAGGGGGATCAGTATCATCTTGGCCTGATCCTGTCTGATTCCTGGATACACGGCTCTCA TGAAAGCATCATATTGCTTGAAAGCCTGCTGGGCTTTACTACCCTCGGGATAAAACATCCCGCAGGACCTGCTCGAA AACTGGTTAGCCTGCACAGCCGGCATCATTCACACAGCAGCGGGCGTCATTGTTGGCTATTTGCACCACACTTCTGC CCCAGCGGTTTTGGGTGATTTTGGTTCGCTCGGGATTCTCCTTTAAGGCTCGTTGTCCGTTCTCGCTGGCCACATCC ATCTCGATAATCTGCTCCTTCTGAATCATAATATTGCCATGCAGGCACTTCAGCTTGCCCTCATAATCATTGCAGCC ATGAGGCCACAACGCACAGCCTGTACATTCCCAATTATGGTGGGCGATCTGAGAAAAAGAATGTATCATTCCCTGCA GAAATCTTCCCATCATCGTGCTCAGTGTCTTGTGACTAGTGAAAGTTAACTGGATGCCTCGGTGCTCTTCGTTTACG TACTGGTGACAGATGCGCTTGTATTGTTCGTGTTGCTCAGGCATTAGTTTAAAACAGGTTCTAAGTTCGTTATCCAG CCTGTACTTCTCCATCAGCAGACACATCACTTCCATGCCTTTCTCCCAAGCAGACACCAGGGGCAAGCTAATCGGAT TCTTAACAGTGCAGGCAGCAGCTCCTTTAGCCAGAGGGTCATCTTTAGCGATCTTCTCAATGCTTCTTTTGCCATCC TTCTCAACGATGCGCACGGGCGGGTAGCTGAAACCCACTGCTACAAGTTGCGCCTCTTCTCTTTCTTCTTCGCTGTC TTGACTGATGTCTTGCATGGGGATATGTTTGGTCTTCCTTGGCTTCTTTTTGGGGGGTATCGGAGGAGGAGGACTGT CGCTCCGTTCCGGAGACAGGGAGGATTGTGACGTTTCGCTCACCATTACCAACTGACTGTCGGTAGAAGAACCTGAC CCCACACGGCGACAGGTGTTTTTCTTCGGGGGCAGAGGTGGAGGCGATTGCGAAGGGCTGCGGTCCGACCTGGAAGG CGGATGACTGGCAGAACCCCTTCCGCGTTCGGGGGTGTGCTCCCTGTGGCGGTCGCTTAACTGATTTCCTTCGCGGC TGGCCATTGTGTTCTCCTAGGCAGAGAAACAACAGACATGGAAACTCAGCCATTGCTGTCAACATCGCCACGAGTGC CATCACATCTCGTCCTCAGCGACGAGGAAAAGGAGCAGAGCTTAAGCATTCCACCGCCCAGTCCTGCCACCACCTCT ACCCTAGAAGATAAGGAGGTCGACGCATCTCATGACATGCAGAATAAAAAAGCGAAAGAGTCTGAGACAGACATCGA GCAAGACCCGGGCTATGTGACACCGGTGGAACACGAGGAAGAGTTGAAACGCTTTCTAGAGAGAGAGGATGAAAACT GCCCAAAACAGCGAGCAGATAACTATCACCAAGATGCTGGAAATAGGGATCAGAACACCGACTACCTCATAGGGCTT GACGGGGAAGACGCGCTCCTTAAACATCTAGCAAGACAGTCGCTCATAGTCAAGGATGCATTATTGGACAGAACTGA AGTGCCCATCAGTGTGGAAGAGCTCAGCTGCGCCTACGAGCTTAACCTTTTTTCACCTCGTACTCCCCCCAAACGTC AGCCAAACGGCACCTGCGAGCCAAATCCTCGCTTAAACTTTTATCCAGCTTTTGCTGTGCCAGAAGTACTGGCTACC TATCACATCTTTTTTAAAAATCAAAAAATTCCAGTCTCCTGCCGCGCTAATCGCACCCGCGCCGATGCCCTACTCAA TCTGGGACCTGGTTCACGCTTACCTGATATAGCTTCCTTGGAAGAGGTTCCAAAGATCTTCGAGGGTCTGGGCAATA ATGAGACTCGGGCCGCAAATGCTCTGCAAAAGGGAGAAAATGGCATGGATGAGCATCACAGCGTTCTGGTGGAATTG GAAGGCGATAATGCCAGACTCGCAGTACTCAAGCGAAGCGTCGAGGTCACACACTTCGCATATCCCGCTGTCAACCT GCCCCCTAAAGTCATGACGGCGGTCATGGACCAGTTACTCATTAAGCGCGCAAGTCCCCTTTCAGAAGACATGCATG ACCCAGATGCCTGTGATGAGGGTAAACCAGTGGTCAGTGATGAGCAGCTAACCCGATGGCTGGGCACCGACTCTCCC CGGGATTTGGAAGAGCGTCGCAAGCTTATGATGGCCGTGGTGCTGGTTACCGTAGAACTAGAGTGTCTCCGACGTTT CTTTACCGATTCAGAAACCTTGCGCAAACTCGAAGAGAATCTGCACTACACTTTTAGACACGGCTTTGTGCGGCAGG CATGCAAGATATCTAACGTGGAACTCACCAACCTGGTTTCCTACATGGGTATTCTGCATGAGAATCGCCTAGGACAA AGCGTGCTGCACAGCACCCTTAAGGGGGAAGCCCGCCGTGATTACATCCGCGATTGTGTCTATCTCTACCTGTGCCA CACGTGGCAAACCGGCATGGGTGTATGGCAGCAATGTTTAGAAGAACAGAACTTGAAAGAGCTTGACAAGCTCTTAC AGAAATCTCTTAAGGTTCTGTGGACAGGGTTCGACGAGCGCACCGTCGCTTCCGACCTGGCAGACCTCATCTTCCCA GAGCGTCTCAGGGTTACTTTGCGAAACGGATTGCCTGACTTTATGAGCCAGAGCATGCTTAACAATTTTCGCTCTTT CATCCTGGAACGCTCCGGTATCCTGCCCGCCACCTGCTGCGCACTGCCCTCCGACTTTGTGCCTCTCACCTACCGCG AGTGCCCCCCGCCGCTATGGAGTCACTGCTACCTGTTCCGTCTGGCCAACTATCTCTCCTACCACTCGGATGTGATC GAGGATGTGAGCGGAGACGGCTTGCTGGAGTGCCACTGCCGCTGCAATCTGTGCACGCCCCACCGGTCCCTAGCTTG CAACCCCCAGTTGATGAGCGAAACCCAGATAATAGGCACCTTTGAATTGCAAGGCCCCAGCAGCCAAGGCGATGGGT CTTCTCCTGGGCAAAGTTTAAAACTGACCCCGGGACTGTGGACCTCCGCCTACTTGCGCAAGTTTGCTCCGGAAGAT TACCACCCCTATGAAATCAAGTTCTATGAGGACCAATCACAGCCTCCAAAGGCCGAACTTTCGGCTTGCGTCATCAC CCAGGGGGCAATTCTGGCCCAATTGCAAGCCATCCAAAAATCCCGCCAAGAATTTCTACTGAAAAAGGGTAAGGGGG TCTACCTTGACCCCCAGACCGGCGAGGAACTCAACACAAGGTTCCCTCAGGATGTCCCAACGACGAGAAAACAAGAA GTTGAAGGTGCAGCCGCCGCCCCCAGAAGATATGGAGGAAGATTGGGACAGTCAGGCAGAGGAGGCGGAGGAGGACA GTCTGGAGGACAGTCTGGAGGAAGACAGTTTGGAGGAGGAAAACGAGGAGGCAGAGGAGGTGGAAGAAGTAACCGCC GACAAACAGTTATCCTCGGCTGCGGAGACAAGCAACAGCGCTACCATCTCCGCTCCGAGTCGAGGAACCCGGCGGCG TCCCAGCAGTAGATGGGACGAGACCGGACGCTTCCCGAACCCAACCAGCGCTTCCAAGACCGGTAAGAAGGATCGGC AGGGATACAAGTCCTGGCGGGGGCATAAGAATGCCATCATCTCCTGCTTGCATGAGTGCGGGGGCAACATATCCTTC ACGCGGCGCTACTTGCTATTCCACCATGGGGTGAACTTTCCGCGCAATGTTTTGCATTACTACCGTCACCTCCACAG CCCCTACTATAGCCAGCAAATCCCGACAGTCTCGACAGATAAAGACAGCGGCGGCGACCTCCAACAGAAAACCAGCA GCGGCAGTTAGAAAATACACAACAAGTGCAGCAACAGGAGGATTAAAGATTACAGCCAACGAGCCAGCGCAAACCCG AGAGTTAAGAAATCGGATCTTTCCAACCCTGTATGCCATCTTCCAGCAGAGTCGGGGTCAAGAGCAGGAACTGAAAA TAAAAAACCGATCTCTGCGTTCGCTCACCAGAAGTTGTTTGTATCACAAGAGCGAAGATCAACTTCAGCGCACTCTC GAGGACGCCGAGGCTCTCTTCAACAAGTACTGCGCGCTGACTCTTAAAGAGTAGGCAGCGACCGCGCTTATTCAAAA AAGGCGGGAATTACATCATCCTCGACATGAGTAAAGAAATTCCCACGCCTTACATGTGGAGTTATCAACCCCAAATG GGATTGGCAGCAGGCGCCTCCCAGGACTACTCCACCCGCATGAATTGGCTCAGCGCCGGGCCTTCTATGATTTCTCG AGTTAATGATATACGCGCCTACCGAAACCAAATACTTTTGGAACAGTCAGCTCTTACCACCACGCCCCGCCAACACC TTAATCCCAGAAATTGGCCCGCCGCCCTAGTGTACCAGGAAAGTCCCGCTCCCACCACTGTATTACTTCCTCGAGAC GCCCAGGCCGAAGTCCAAATGACTAATGCAGGTGCGCAGTTAGCTGGCGGCTCCACCCTATGTCGTCACAGGCCTCG GCATAATATAAAACGCCTGATGATCAGAGGCCGAGGTATCCAGCTCAACGACGAGTCGGTGAGCTCTCCGCTTGGTC TACGACCAGACGGAATCTTTCAGATTGCCGGCTGCGGGAGATCTTCCTTCACCCCTCGTCAGGCTGTTCTGACTTTG GAAAGTTCGTCTTCGCAACCCCGCTCGGGCGGAATCGGGACCGTTCAATTTGTAGAGGAGTTTACTCCCTCTGTCTA CTTCAACCCCTTCTCCGGATCTCCTGGGCACTACCCGGACGAGTTCATACCGAACTTCGACGCGATTAGCGAGTCAG TGGACGGCTACGATTGATGTCTGGTGACGCGGCTGAGCTATCTCGGCTGCGACATCTAGACCACTGCCGCCGCTTTC GCTGCTTTGCCCGGGAACTTATTGAGTTCATCTACTTCGAACTCCCCAAGGATCACCCTCAAGGTCCGGCCCACGGA GTGCGGATTACTATCGAAGGCAAAATAGACTCTCGCCTGCAACGAATTTTCTCCCAGCGGCCCGTGCTGATCGAGCG AGACCAGGGAAACACCACGGTTTCCATCTACTGCATTTGTAATCACCCCGGATTGCATGAAAGCCTTTGCTGTCTTA TGTGTACTGAGTTTAATAAAAACTGAATTAAGACTCTCCTACGGACTGCCGCTTCTTCAACCCGGATTTTACAACCA GAAGAACAAAACTTTTCCTGTCGTCCAGGACTCTGTTAACTTCACCTTTCCTACTCACAAACTAGAAGCTCAACGAC TACACCGCTTTTCCAGAAGCATTTTCCCTACTAATACTACTTTCAAAACCGGAGGTGAGCTCCACGGTCTCCCTACA GAAAACCCTTGGGTGGAAGCGGGCCTTGTAGTACTAGGAATTCTTGCGGGTGGGCTTGTGATTATTCTTTGCTACCT ATACACACCTTGCTTCACTTTCCTAGTGGTGTTGTGGTATTGGTTTAAAAAATGGGGCCCATACTAGTCTTGCTTGT TTTACTTTCGCTTTTGGAACCGGGTTCTGCCAATTACGATCCATGTCTAGACTTTGACCCAGAAAACTGCACACTTA CTTTTGCACCCGACACAAGCCGCATCTGTGGAGTTCTTATTAAGTGCGGATGGGAATGCAGGTCCGTTGAAATTACA CACAATAACAAAACCTGGAACAATACCTTATCCACCACATGGGAGCCAGGAGTTCCCGAGTGGTACACTGTCTCTGT CCGAGGTCCTGACGGTTCCATCCGCATTAGTAACAACACTTTCATTTTTTCTGAAATGTGCGATCTGGCCATGTTCA TGAGCAAACAGTATTCTCTATGGCCTCCTAGCAAGGACAACATCGTAACGTTCTCCATTGCTTATTGCTTGTGCGCT TGCCTTCTTACTGCTTTACTGTGCGTATGCATACACCTGCTTGTAACCACTCGCATCAAAAACGCCAATAACAAAGA AAAAATGCCTTAACCTCTTTCTGTTTACAGACATGGCTTCTCTTACATCTCTCATATTTGTCAGCATTGTCACTGCC GCTCACGGACAAACAGTCGTCTCTATCCCACTAGGACATAATTACACTCTCATAGGACCCCCAATCACTTCAGAGGT CATCTGGACCAAACTGGGAAGCGTTGATTACTTTGATATAATCTGTAACAAAACAAAACCAATAATAGTAACTTGCA ACATACAAAATCTTACATTGATTAATGTTAGCAAAGTTTACAGCGGTTACTATTATGGTTATGACAGATACAGTAGT CAATATAGAAATTACTTGGTTCGTGTTACCCAGTTGAAAACCACGAAAATGCCAAATATGGCAAAGATTCGATCCGA TGACAATTCTCTAGAAACTTTTACATCTCCCACCACACCCGACGAAAAAAACATCCCAGATTCAATGATTGCAATTG TTGCAGCGGTGGCAGTGGTGATGGCACTAATAATAATATGCATGCTTTTATATGCTTGTCGCTACAAAAAGTTTCAT CCTAAAAAACAAGATCTCCTACTAAGGCTTAACATTTAATTTCTTTTTATACAGCCATGGTTTCCACTACCACATTC CTTATGCTTACTAGTCTCGCAACTCTGACTTCTGCTCGCTCACACCTCACTGTAACTATAGGCTCAAACTGCACACT AAAAGGACCTCAAGGTGGTCATGTCTTTTGGTGGAGAATATATGACAATGGATGGTTTACAAAACCATGTGACCAAC CTGGTAGATTTTTCTGCAACGGCAGAGACCTAACCATTATCAACGTGACAGCAAATGACAAAGGCTTCTATTATGGA ACCGACTATAAAAGTAGTTTAGATTATAACATTATTGTACTGCCATCTACCACTCCACCACCCCGCACAACTACTTT CTCTAGCAGCAGTGTCGCTAACAATACAATTTCCAATCCAACCTTTGCCGCGCTTTTAAAACGCACTGTGAATAATT CTACAACTTCACATACAACAATTTCCACTTCAACAATCAGCATCATCGCTGCAGTGACAATTGGAATATCTATTCTT GTTTTTACCATAACCTACTACGCCTGCTGCTATAGAAAAGACAAACATAAAGGTGATCCATTACTTAGATTTGATAT TTAATTTGTTCTTTTTTTTTATTTACAGTATGGTGAACACCAATCATGGTACCTAGAAATTTCTTCTTCACCATACT CATCTGTGCTTTTAATGTTTGCGCTACTTTCACAGCAGTAGCCACAGCAACCCCAGACTGTATAGGAGCATTTGCTT CCTATGCACTTTTTGCTTTTGTTACTTGCATCTGCGTATGTAGCATAGTCTGCCTGGTTATTAATTTTTTCCAACTT CTAGACTGGATCCTTGTGCGAATTGCCTACCTGCGCCACCATCCCGAATACCGCAACCAAAATATCGCGGCACTTCT TAGACTCATCTAAAACCATGCAGGCTATACTACCAATATTTTTGCTTCTATTGCTTCCCTACGCTGTCTCAACCCCA GCTGCCTATAGTACTCCACCAGAACACCTTAGAAAATGCAAATTCCAACAACCGTGGTCATTTCTTGCTTGCTATCG AGAAAAATCAGAAATCCCCCCAAATTTAATAATGATTGCTGGAATAATTAATATAATCTGTTGCACCATAATTTCAT TTTTGATATACCCCCTATTTGATTTTGGCTGGAATGCTCCCAATGCACATGATCATCCACAAGACCCAGAGGAACAC ATTCCCCCACAAAACATGCAACATCCAATAGCGCTAATAGATTACGAAAGTGAACCACAACCCCCACTACTCCCTGC TATTAGTTACTTCAACCTAACCGGCGGAGATGACTGAAACACTCACCACCTCCAATTCCGCCGAGGATCTGCTCGAT ATGGACGGCCGCGTCTCAGAACAACGACTTGCCCAACTACGCATCCGCCAGCAGCAGGAACGCGTGGCCAAAGAGCT CAGAGATGTCATCCAAATTCACCAATGCAAAAAAGGCATATTCTGTTTGGTAAAACAAGCCAAGATATCCTACGAGA TCACCGCTACTGACCATCGCCTCTCTTACGAACTTGGCCCCCAACGACAAAAATTTACCTGCATGGTGGGAATCAAC CCCATAGTTATCACCCAACAAAGTGGAGATACTAAGGGTTGCATTCACTGTTCCTGCGATTCCATCGAGTGCACCTA CACCCTGCTGAAGACCCTATGCGGCCTAAGAGACCTGCTACCAATGAATTAAAAAAAAATGATTAATAAAAAATCAC TTACTTGAAATCAGCAATAAGGTCTCTGTTGAAATTTTCTCCCAGCAGCACCTCACTTCCCTCTTCCCAACTCTGGT ATTCTAAACCCCGTTCAGCGGCATACTTTCTCCATACTTTAAAGGGGATGTCAAATTTTAGCTCCTCTCCTGTACCC ACAATCTTCATGTCTTTCTTCCCAGATGACCAAGAGAGTCCGGCTCAGTGACTCCTTCAACCCTGTCTACCCCTATG AAGATGAAAGCACCTCCCAACACCCCTTTTATAACCCAGGGTTTATTTCCCCAAATGGCTTCACACAAAGCCCAGAC GGAGTTCTTACTTTAAAATGTTTAACCCCACTAACAACCACAGGCGGATCTCTACAGCTAAAAGTGGGAGGGGGACT TACAGTGGATGACACTGATGGTACCTTACAAGAAAACATACGTGCTACAGCACCCATTACTAAAAATAATCACTCTG TAGAACTATCCATTGGAAATGGATTAGAAACTCAAAACAATAAACTATGTGCCAAATTGGGAAATGGGTTAAAATTT AACAACGGTGACATTTGTATAAAGGATAGTATTAACACCTTATGGACTGGAATAAACCCTCCACCTAACTGTCAAAT TGTGGAAAACACTAATACAAATGATGGCAAACTTACTTTAGTATTAGTAAAAAATGGAGGGCTTGTTAATGGCTACG TGTCTCTAGTTGGTGTATCAGACACTGTGAACCAAATGTTCACACAAAAGACAGCAAACATCCAATTAAGATTATAT TTTGACTCTTCTGGAAATCTATTAACTGAGGAATCAGACTTAAAAATTCCACTTAAAAATAAATCTTCTACAGCGAC CAGTGAAACTGTAGCCAGCAGCAAAGCCTTTATGCCAAGTACTACAGCTTATCCCTTCAACACCACTACTAGGGATA GTGAAAACTACATTCATGGAATATGTTACTACATGACTAGTTATGATAGAAGTCTATTTCCCTTGAACATTTCTATA ATGCTAAACAGCCGTATGATTTCTTCCAATGTTGCCTATGCCATACAATTTGAATGGAATCTAAATGCAAGTGAATC TCCAGAAAGCAACATAGCTACGCTGACCACATCCCCCTTTTTCTTTTCTTACATTACAGAAGACGACAACTAAAATA AAGTTTAAGTGTTTTTATTTAAAATCACAAAATTCGAGTAGTTATTTTGCCTCCACCTTCCCATTTGACAGAATACA CCAATCTCTCCCCACGCACAGCTGTTAAACATTTGGATACCATTAGAGATAGACATTGTTTTAGATTCCACATTCCA AACAGTTTCAGAGCGAGCCAATCTGGGGTCAGTGATAGATAAAAATCCATCGCGATAGTCTTTTAAAGCGCTTTCAC AGTCCAACTGCTGCGGATGCGACTCCGGAGTTTGGATCACGGTCATCTGGAAGAAGAACGATGGGAATCATAATCCG AAAACGGTATCGGACGATTGTGTCTCATCAAACCCACAAGCAGCCGCTGTCTGCGTCGCTCCGTGCGACTGCTGTTT ATGGGATCAGGGTCCACAGTTTCCTGAAGCATGATTTTAATAGCCCTTAACATCAACTTTCTGGTGCGATGCGCGCA GCAACGCATTCTGATTTCACTCAAATCTTTGCAGTAGGTACAACACATTATTACAATATTGTTTAATAAACCATAAT TAAAAGCGCTCCAGCCAAAACTCATATCTGATATAATCGCCCCTGCATGACCATCATACCAAAGTTTAATATAAATT AAATGACGTTCCCTCAAAAACACACTACCCACATACATGATCTCTTTTGGCATGTGCATATTAACAATCTGTCTGTA CCATGGACAACGTTGGTTAATCATGCAACCCAATATAACCTTCCGGAACCACACTGCCAACACCGCTCCCCCAGCCA TGCATTGAAGTGAACCCTGCTGATTACAATGACAATGAAGAACCCAATTCTCTCGACCGTGAATCACTTGAGAATGA AAAATATCTATAGTGGCACAACATAGACATAAATGCATGCATCTTCTCATAATTTTTAACTCCTCAGGATTTAGAAA CATATCCCAGGGAATAGGAAGCTCTTGCAGAACAGTAAAGCTGGCAGAACAAGGAAGACCACGAACACAACTTACAC TATGCATAGTCATAGTATCACAATCTGGCAACAGCGGGTGGTCTTCAGTCATAGAAGCTCGGGTTTCATTTTCCTCA CAACGTGGTAACTGGGCTCTGGTGTAAGGGTGATGTCTGGCGCATGATGTCGAGCGTGCGCGCAACCTTGTCATAAT GGAGTTGCTTCCTGACATTCTCGTATTTTGTATAGCAAAACGCGGCCCTGGCAGAACACACTCTTCTTCGCCTTCTA TCCTGCCGCTTAGCGTGTTCCGTGTGATAGTTCAAGTACAGCCACACTCTTAAGTTGGTCAAAAGAATGCTGGCTTC AGTTGTAATCAAAACTCCATCGCATCTAATTGTTCTGAGGAAATCATCCACGGTAGCATATGCAAATCCCAACCAAG CAATGCAACTGGATTGCGTTTCAAGCAGGAGAGGAGAGGGAAGAGACGGAAGAACCATGTTAATTTTTATTCCAAAC GATCTCGCAGTACTTCAAATTGTAGATCGCGCAGATGGCATCTCTCGCCCCCACTGTGTTGGTGAAAAAGCACAGCT AAATCAAAAGAAATGCGATTTTCAAGGTGCTCAACGGTGGCTTCCAACAAAGCCTCCACGCGCACATCCAAGAACAA AAGAATACCAAAAGAAGGAGCATTTTCTAACTCCTCAATCATCATATTACATTCCTGCACCATTCCCAGATAATTTT CAGCTTTCCAGCCTTGAATTATTCGTGTCAGTTCTTGTGGTAAATCCAATCCACACATTACAAACAGGTCCCGGAGG GCGCCCTCCACCACCATTCTTAAACACACCCTCATAATGACAAAATATCTTGCTCCTGTGTCACCTGTAGCGAATTG AGAATGGCAACATCAATTGACATGCCCTTGGCTCTAAGTTCTTCTTTAAGTTCTAGTTGTAAAAACTCTCTCATATT ATCACCAAACTGCTTAGCCAGAAGCCCCCCGGGAACAAGAGCAGGGGACGCTACAGTGCAGTACAAGCGCAGACCTC CCCAATTGGCTCCAGCAAAAACAAGATTGGAATAAGCATATTGGGAACCACCAGTAATATCATCGAAGTTGCTGGAA ATATAATCAGGCAGAGTTTCTTGTAGAAATTGAATAAAAGAAAAATTTGCCAAAAAAACATTCAAAACCTCTGGGAT GCAAATGCAATAGGTTACCGCGCTGCGCTCCAACATTGTTAGTTTTGAATTAGTCTGCAAAAATAAAAAAAAAACAA GCGTCATATCATAGTAGCCTGACGAACAGGTGGATAAATCAGTCTTTCCATCACAAGACAAGCCACAGGGTCTCCAG CTCGACCCTCGTAAAACCTGTCATCGTGATTAAACAACAGCACCGAAAGTTCCTCGCGGTGACCAGCATGAATAAGT CTTGATGAAGCATACAATCCAGACATGTTAGCATCAGTTAAGGAGAAAAAACAGCCAACATAGCCTTTGGGTATAAT TATGCTTAATCGTAAGTATAGCAAAGCCACCCCTCGCGGATACAAAGTAAAAGGCACAGGAGAATAAAAAATATAAT TATTTCTCTGCTGCTGTTTAGGCAACGTCGCCCCCGGTCCCTCTAAATACACATACAAAGCCTCATCAGCCATGGCT TACCAGAGAAAGTACAGCGGGCACACAAACCACAAGCTCTAAAGTCACTCTCCAACCTCTCCACAATATATATACAC AAGCCCTAAACTGACGTAATGGGACTAAAGTGTAAAAAATCCCGCCAAACCCAACACACACCCCGAAACTGCGTCAC CAGGGAAAAGTACAGTTTCACTTCCGCAATCCCAACAAGCGTCACTTCCTCTTTCTCACGGTACGTCACATCCCATT AACTTACAACGTCATTTTCCCACGGCCGCGCCGCCCCTTTTAACCGTTAACCCCACAGCCAATCACCACACGGCCCA CACTTTTTAAAATCACCTCATTTACATATTGGCACCATTCCATCTATAAGGTATATTATTGATGATG

GenBank Accession No. AP_000580 MRRVVLGGAVVYPEGPPPSYESVMQQQQATAVMOSPLEAPFVPPRYLAPTEGRNSIRYSELAPQYDTTRLYLVDNKS ADIASLNYQNDHSNFLTTVVONNDFTPTEASTQTINFDERSRWGGOLKTIMHTNMPNVNEYMESNKFKARVMVSRKP PDGAAVGDTYDHKQDILEYEWFEFTLPEGNESVIMTIDLMNNAIIDNYLKVGRONGVLESDIGVKFDTRNFKLGWDP ETKLIMPGVYTYEAFHPDIVLLPGCGVDFTESRLSNLLGIRKKOPFQEGFKILYEDLEGGNIPALLDVDAYENSKKE QKAKIEAATAAAEAKANIVASDSTRVANAGEVRGDNFAPTPVPTAESLLADVSEGTDVKLTIQPVEKDSKNRSYNVL EDKINTAYRSWYLSYNYGDPEKGVRSWTLLTTSDVTCGAEQVYWSLPDMMKDPVTFRSTRQVSNYPVVGAELMPVFS KSFYNEQAVYSQQLRQSTSLTHVENRFPENQILIRPPAPTITTVSENVPALTDHGTLPLRSSIRGVQRVTVTDARRR TCPYVYKALGIVAPRVLSSRTF

GenBank Accession No. AP_000585 MATPSMLPQWAYMHIAGQDASEYLSPGLVQFARATDTYENLGNKFRNPTVAPTHDVTTDRSQRLMLRFVPVDREDNT YSYKVRYTLAVGDNRVLDMASTFFDIRGVLDRGPSFKPYSGTAYNSLAPKGAPNASQWIAKGVPTAAAAGNGEEEHE TEEKTATYTFANAPVKAEAQITKEGLPIGLEISAENESKPIYADKLYQPEPQVGDETWTDLDGKTEEYGGRALKPTT NMKPCYGSYAKPTNLKGGQAKPKNSEPSSEKIEYDIDMEFFDNSSQRTNESPKIVMYAENVGLETPDTHVVYKPGTE DTSSEANLGQQSMPNRPNYIGFRDNFIGLMYYNSTGNMGVLAGQASQLNAVVDLQDRNTELSYQLLLDSLGDRTRYF SMWNQAVDSYDPDVRVIENHGVEDELPNYCFPLDGIGVPTTSYKSIVPNGEDNNNWKEPEVNGTSEIGQGNLFAMEI NLQANLWRSFLYSNVALYLPDSYKYTPSNVTLPENKNTYDYMNGRVVPPSLVDTYVNIGARWSLDAMDNVNPFNHHR NAGLRYRSMLLGNGRYVPFHIQVPQKFFAVKNLLLLPGSYTYEWNFRKDVNMVLQSSLGNDLRVDGASISFTSINLY ATFFPMAHNTASTLEAMLRNDTNDQSENDYLSAANMLYPIPANATNIPISIPSRNWAAFRGWSFTRLKTKETPSLGS GFDPYFVYSGSIPYLDGTFYLNHTFKKVSIMFDSSVSWPGNDRLLSPNEFEIKRTVDGEGYNVAQCNMTKDWFLVQM LANYNIGYQGFYIPEGYKDRMYSFFRNFQPMSRQVVDEVNYKDFKAVAIPYQHNNSGFVGYMAPTMRQGQPYPANYP YPLIGTTAVNSVTQKKFLCDRTMWRIPFSSNFMSMGALTDLGONMLYANSAHALDMTFEVDPMDEPTLLYLLFEVED VVRVHQPHRGIIEAVYLRTPFSAGNATT

### OTHER EMBODIMENTS

It will be appreciated that the scope of the present disclosure is to be defined by that which may be understood from the disclosure and claims rather than by the specific embodiments that have been presented by way of example. Elements described with respect to one aspect or embodiment of the present disclosure are also contemplated with respect to other aspects or embodiments of the present disclosure. For example, elements of claims that depend directly or indirectly from a certain independent claim presented herein serve as support for those elements being presented in additional dependent claims of one or more other independent claims. Throughout the description, where compositions or methods are described as having, including, or comprising specific elements, compositions that consist essentially of, consist of, or do not comprise the recited elements. All references cited herein are hereby incorporated by reference.

What is claimed is:

1. An O⁶BG-resistant O(6)-methylguanine-DNA-methyltransferase (MGMT) polypeptide, wherein the O⁶BG-resistant MGMT polypeptide comprises one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

2. A nucleic acid encoding an O⁶BG-resistant O(6)-methylguanine-DNA-methyltransferase (MGMT) polypeptide, wherein the O⁶BG-resistant MGMT polypeptide comprises one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

3. The nucleic acid of claim 2, wherein each of the one or more amino acid mutations is encoded in the nucleic acid by a corresponding nucleic acid mutation selected from Table 1.

4. A cell comprising the polypeptide of claim 1.

5. A cell comprising the nucleic acid of claim 2 or 3.

6. The cell of claim 4 or 5, wherein the cell is a mammalian cell, optionally wherein the mammalian cell is a human cell.

7. The cell of any one of claims 4-6, wherein the cell is a hematopoietic cell, optionally wherein the hematopoietic cell is a hematopoietic stem and progenitor cell (HSPC) or a hematopoietic stem cell (HSC).

8. The cell of any one of claims 4-7, wherein the cell comprises a therapeutic gene and/or encode a therapeutic expression product.

9. A pharmaceutical composition comprising the cell of any one of claims 4-8.

10. A method of hematopoietic stem cell (HSC) transplantation comprising administering the cell of any one of claims 4-8 or the pharmaceutical composition of claim 9 to a subject in need thereof.

11. Use of the cell of any one of claims 4-8 or the pharmaceutical composition of claim 9 in hematopoietic stem cell (HSC) transplantation.

12. The method or use of claim 10 or claim 11, wherein the HSC transplantation is an autologous therapy in that the cells are derived from cells of the subject.

13. The method or use of claim 10 or claim 11, wherein the HSC transplantation is an allogeneic therapy in that the cells are not derived from cells of the subject.

14. A method comprising contacting an endogenous O(6)-methylguanine-DNA-methyltransferase (MGMT)-encoding nucleic acid of one or more cells of a mammalian subject with an editing enzyme to produce a modified MGMT-encoding nucleic acid, wherein the contacting occurs in vivo, in vitro, or ex vivo and the modified MGMT-encoding nucleic acid encodes an O⁶-benzylguanine (O⁶BG)-resistant MGMT polypeptide,
wherein the O⁶BG-resistant MGMT polypeptide is MGMT^{P140K}.

15. A method comprising contacting an endogenous O(6)-methylguanine-DNA-methyltransferase (MGMT)-encoding nucleic acid of one or more cells of a mammalian subject with an editing enzyme to produce a modified MGMT-encoding nucleic acid, wherein the contacting occurs in vivo, in vitro, or ex vivo and the modified MGMT-encoding nucleic acid encodes an O⁶-benzylguanine (O⁶BG)-resistant MGMT polypeptide,
wherein the O⁶BG-resistant MGMT polypeptide is not MGMT^{P140K} and/or does not comprise a lysine (K) at position 140 corresponding to SEQ ID NO: 1.

16. The method of claim 15, wherein the O⁶BG-resistant MGMT polypeptide comprises one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK.

17. The method of claim 15, wherein the O⁶BG-resistant MGMT polypeptide comprises one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

18. The method of claim 16 or 17, wherein each of the one or more amino acid mutations is encoded in the modified MGMT-encoding nucleic acid by a corresponding nucleic acid mutation selected from Table 1 and/or Table 2.

19. The method of any one of claims 14-18, wherein the contacting occurs in vivo.

20. Use of an editing enzyme for modification of an endogenous O(6)-methylguanine-DNA-methyltransferase (MGMT)-encoding nucleic acid of one or more cells of a mammalian subject to produce a modified MGMT-encoding nucleic acid,
wherein the use comprises contacting the endogenous MGMT-encoding nucleic acid with the editing enzyme in vivo, in vitro, or ex vivo and the modified MGMT-encoding nucleic acid encodes an O⁶-benzylguanine (O⁶BG)-resistant MGMT polypeptide,
wherein the O⁶BG-resistant MGMT polypeptide is MGMT^{P140K}.

21. Use of an editing enzyme for modification of an endogenous O(6)-methylguanine-DNA-methyltransferase (MGMT)-encoding nucleic acid of one or more cells of a mammalian subject to produce a modified MGMT-encoding nucleic acid,
wherein the use comprises contacting the endogenous MGMT-encoding nucleic acid with the editing enzyme in vivo, in vitro, or ex vivo and the modified MGMT-encoding nucleic acid encodes an O⁶-benzylguanine (O⁶BG)-resistant MGMT polypeptide,
wherein the O⁶BG-resistant MGMT polypeptide is not MGMT^{P140K} and/or does not comprise a lysine (K) at position 140 corresponding to SEQ ID NO: 1.

22. The use of claim 21, wherein the O⁶BG-resistant MGMT polypeptide comprises one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK.

23. The use of claim 21, wherein the O⁶BG-resistant MGMT polypeptide comprises one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

24. The use of claim 22 or 23, wherein each of the one or more amino acid mutations is encoded in the modified MGMT-encoding nucleic acid by a corresponding nucleic acid mutation selected from Table 1 and/or Table 2.

25. The use of any one of claims 20-24, wherein the contacting occurs in vivo.

26. The method or use of any one of claims 14-25, comprising administering to the mammalian subject a nucleic acid encoding the editing enzyme.

27. The method or use of claim 26, wherein the nucleic acid encoding the editing enzyme further encodes a guide RNA that directs editing of the endogenous MGMT-encoding nucleic acid by the editing enzyme.

28. The method or use of claim 26 or claim 27, wherein the nucleic acid encoding the editing enzyme is administered parenterally.

29. The method or use of any one of claims 26-28, wherein the nucleic acid encoding the editing enzyme is administered by injection.

30. The method or use of any one of claims 26-29, wherein the nucleic acid encoding the editing enzyme is administered intravenously.

31. The method or use of claim any one of claims 14-30, comprising mobilization of hematopoietic stem cells of the subject prior to administration of the nucleic acid.

32. The method or use of any one of claims 14-31, comprising administering one or more immunosuppression agents to the subject, optionally wherein the administration of the one or more immunosuppression agents is prior to the administration of the nucleic acid.

33. The method or use of any one of claims 14-32, comprising administering one or more MGMT inhibitors to the subject after the nucleic acid has been administered.

34. The method or use of any one of claims 14-33, wherein the one or more MGMT inhibitors comprises O⁶BG or an analog or derivative thereof, and/or wherein the one or more MGMT inhibitors comprises Lomeguatrib.

35. The method or use of any one of claims 14-34, comprising administering one or more alkylating agents to the subject after the nucleic acid has been administered.

36. The method or use of any one of claims 14-35, wherein the one or more alkylating agent comprises 1,3-bis(2-chloroethyl)-1-nitrosourea (BCNU) or temozolomide.

37. The method or use of any one of claims 14-36, wherein the modified MGMT-encoding nucleic acid confers a selective advantage to, and/or permits selection of, cells comprising the modified MGMT-encoding nucleic acid.

38. The method or use of any one of claims 14-37, comprising selecting for cells comprising the modified MGMT-encoding nucleic acid.

39. A nucleic acid encoding an editing enzyme and optionally further encoding a guide RNA, wherein the editing enzyme, upon contact with an O(6)-methylguanine-DNA-methyltransferase (MGMT)-encoding nucleic acid, produces a modified MGMT-encoding nucleic acid that encodes an O⁶-benzylguanine (O⁶BG)-resistant MGMT polypeptide,
wherein the O⁶BG-resistant MGMT polypeptide is MGMT^{P140K}.

40. A nucleic acid encoding an editing enzyme and optionally further encoding a guide RNA, wherein the editing enzyme, upon contact with an O(6)-methylguanine-DNA-methyltransferase (MGMT)-encoding nucleic acid, produces a modified MGMT-encoding nucleic acid that encodes an O⁶-benzylguanine (O⁶BG)-resistant MGMT polypeptide,
wherein the O⁶BG-resistant MGMT polypeptide is not MGMT^{P140K} and/or does not comprise a lysine (K) at position 140 corresponding to SEQ ID NO: 1.

41. The nucleic acid encoding an editing enzyme of claim 40, wherein the O⁶BG-resistant MGMT polypeptide comprises one or more amino acid mutations selected from V139F, P140A, P140G, P140I, P140L, P140M, P140N, P140Q, P140R, P140S, P140T, L142M, C150Y, S152H, A154G, G156A, N157T, Y158F, Y158H, G160A, G160R, G160S, L162P, L162V, K165E, K165N, K165R, A170S, PVP(138-140)CMK, PVP(138-140)CIK, PVP(138-140)HLK, PVP(138-140)KIK, PVP(138-140)KIR, PVP(138-140)KLK, PVP(138-140)KMK, PVP(138-140)KVK, PVP(138-140)KWK, PVP(138-140)KYK, PVP(138-140)KYN, PVP(138-140)KYR, PVP(138-140)MIK, PVP(138-140)MLK, PVP(138-140)MMK, PVP(138-140)MVK, PVP(138-140)MWK, PVP(138-140)MYR, PVP(138-140)NIK, PVP(138-140)NLK, PVP(138-140)NLL, PVP(138-140)PLK, PVP(138-140)PYR, PVP(138-140)QLN, PVP(138-140)RFK, PVP(138-140)RTK, PVP(138-140)RYK, PVP(138-140)SFK, PVP(138-140)SMK, PVP(138-140)TIK, PVP(138-140)TLK, PVP(138-140)TLN, PVP(138-140)TNK, PVP(138-140)RCK, PVP(138-140)SYK, PVP(138-140)VMK, and PVP(138-140)YAK.

42. The nucleic acid encoding an editing enzyme of claim 40, wherein the O⁶BG-resistant MGMT polypeptide comprises one or more amino acid mutations selected from L33F, L33K, L33P, L33R, L33W, L33Y, M134F, M134V, M134W, M134Y, R135G, R135K, R135L, R135T, N137D, N137F, N137P, P138K, P140E, P140F, P140H, G156I, G156P, G156V, Y158M, Y158W, S159F, S159I, S159L, S159P, S159T, S159W, S159Y, G160D, G160E, G160H, G160K, and G160P.

43. The nucleic acid encoding an editing enzyme of claim 41 or 42, wherein each of the one or more amino acid mutations is encoded in the modified MGMT-encoding nucleic acid by a corresponding nucleic acid mutation selected from Table 1 and/or Table 2.

44. The nucleic acid nucleic acid encoding an editing enzyme of any one of claims 39-43, wherein the nucleic acid encoding the editing enzyme encodes a guide RNA that directs editing of the endogenous MGMT-encoding nucleic acid by the editing enzyme.

45. A pharmaceutical composition comprising the nucleic acid encoding an editing enzyme of any one of claims 39-44.

46. The pharmaceutical composition of claim 45, wherein the pharmaceutical composition is formulated for administration to a mammalian subject, optionally wherein the mammalian subject is a human subject.

47. The pharmaceutical composition of claim 45 or claim 46, wherein the pharmaceutical composition is formulated for parenteral administration.

48. The pharmaceutical composition of any one of claims 45-47, wherein the pharmaceutical composition is formulated for injection.

49. The pharmaceutical composition of any one of claims 45-48, wherein the pharmaceutical composition is formulated for intravenous injection.

50. A kit comprising the nucleic acid encoding an editing enzyme of any one of claims 39-44 or the pharmaceutical composition of any one of claims 45-49.

51. The kit of claim 50, wherein the kit comprises one or more MGMT inhibitors.

52. The kit of claim 51, wherein the one or more MGMT inhibitors comprises O⁶BG or an analog or derivative thereof, and/or wherein the one or more MGMT inhibitors comprises Lomeguatrib.

53. The kit of any one of claims 50-52, wherein the kit comprises one or more alkylating agents.

54. The kit of claim 53, wherein the one or more alkylating agent comprises 1,3-bis(2-chloroethyl)-1-nitrosourea (BCNU) or temozolomide.

55. The kit of any one of claims 50-54, wherein the kit comprises one or more mobilization agents.

56. The kit of any one of claims 50-55, wherein the kit comprises one or more immunosuppression agents.

57. The kit of any one of claims 50-56, wherein the kit comprises instructions for selection for cells comprising modified MGMT-encoding nucleic acids.

58. The method, use, nucleic acid, pharmaceutical composition, or kit of any one of claims 14-57, wherein the editing enzyme is a base editing enzyme that deaminates a nucleobase in the endogenous MGMT-encoding nucleic acid.

59. The method, use, nucleic acid, pharmaceutical composition, or kit of claim 58, wherein the base editing enzyme comprises a DNA binding domain and a deaminase domain.

60. The method, use, nucleic acid, pharmaceutical composition, or kit of claim 59, wherein the DNA binding domain and deaminase domain are fused.

61. The method, use, nucleic acid, pharmaceutical composition, or kit of claim 59 or claim 60, wherein the DNA binding domain is a zinc finger domain.

62. The method, use, nucleic acid, pharmaceutical composition, or kit of claim 59 or claim 60, wherein the DNA binding domain is a TALEN domain.

63. The method, use, nucleic acid, pharmaceutical composition, or kit of claim 59 or claim 60, wherein the DNA binding domain is an RNA guided DNA binding domain.

64. The method, use, nucleic acid, pharmaceutical composition, or kit of claim 63, wherein the RNA guided DNA binding domain is a modified Cas9 variant or a modified Cas12a variant.

65. The method, use, nucleic acid, pharmaceutical composition, or kit of claim 63 or claim 64, wherein the RNA guided DNA binding domain is a catalytically impaired nuclease domain.

66. The method, use, nucleic acid, pharmaceutical composition, or kit of any one of claims 63-65, wherein the RNA guided DNA binding domain is a nickase variant.

67. The method, use, nucleic acid, pharmaceutical composition, or kit of any one of claims 59-66, wherein the deaminase domain is a cytidine deaminase domain.

68. The method, use, nucleic acid, pharmaceutical composition, or kit of any one of claims 59-66, wherein the deaminase domain is an adenosine deaminase domain.

69. The method, use, nucleic acid, pharmaceutical composition, or kit of any one of claims 14-57, wherein the editing enzyme is a prime editing enzyme that comprises a DNA binding domain and a reverse transcriptase domain.

70. The method, use, nucleic acid, pharmaceutical composition, or kit of claim 69, wherein the DNA binding domain is an RNA guided DNA binding domain.

71. The method, use, nucleic acid, pharmaceutical composition, or kit of claim 70, wherein the RNA guided DNA binding domain and reverse transcriptase domain are fused.

72. The method, use, nucleic acid, pharmaceutical composition, or kit of claim 69 or claim 70, wherein the RNA guided DNA binding domain is a modified Cas9 variant or a modified Cas12a variant.

73. The method, use, nucleic acid, pharmaceutical composition, or kit of any one of claims 70-72, wherein the RNA guided DNA binding domain is a catalytically impaired nuclease domain.

74. The method, use, nucleic acid, pharmaceutical composition, or kit of any one of claims 70-73, wherein the RNA guided DNA binding domain is a nickase variant.

75. The method, use, nucleic acid, pharmaceutical composition, or kit of any one of claims 70-74, wherein the reverse transcriptase domain is an MLV reverse transcriptase domain.

76. The method, use, nucleic acid, pharmaceutical composition, or kit of any one of claims 14-57, wherein the editing enzyme is an RNA editing enzyme that deaminates a nucleobase in mRNA transcripts produced from the endogenous MGMT gene to produce a modified MGMT mRNA transcript.

77. The method, use, nucleic acid, pharmaceutical composition, or kit of any one of claims 26-76, wherein nucleic acid encoding the editing enzyme is encapsidated in a viral particle.

78. The method, use, nucleic acid, pharmaceutical composition, or kit of claim 77, wherein the viral particle is a recombinant adenovirus.

79. The method, use, nucleic acid, pharmaceutical composition, or kit of claim 78, wherein the recombinant adenovirus is a recombinant Ad35 virus.

80. The method, use, nucleic acid, pharmaceutical composition, or kit of claim 78, wherein the recombinant adenovirus is a recombinant Ad5 virus.

81. The method, use, nucleic acid, pharmaceutical composition, or kit of claim 78, wherein the recombinant adenovirus is an Ad3, Ad7, Ad11, Ad14, Ad16, Ad21, Ad34, Ad37, or Ad50 virus.

82. The method, use, nucleic acid, pharmaceutical composition, or kit of claim 78, wherein the recombinant adenovirus is a chimeric adenovirus.

83. The method, use, nucleic acid, pharmaceutical composition, or kit of claim 82, wherein the chimeric adenovirus is an Ad5/35 virus (e.g., an Ad5/35++ virus).

84. The method, use, nucleic acid, pharmaceutical composition, or kit of any one of claims 26-76, wherein nucleic acid encoding the editing enzyme is encapsulated in a lipid nanoparticle.

85. The method, use, nucleic acid, pharmaceutical composition, or kit of any one of claims 26-76, wherein nucleic acid encoding the editing enzyme is encapsulated in a liposome.

86. The method, use, nucleic acid, pharmaceutical composition, or kit of any one of claims 26-85, wherein the nucleic acid further comprises a therapeutic payload.

87. The method, use, nucleic acid, pharmaceutical composition, or kit of claim 86, wherein the therapeutic payload is a non-integrating payload.

88. The method, use, nucleic acid, pharmaceutical composition, or kit of claim 86, wherein the therapeutic payload is an integrating payload, optionally wherein the integrating payload does not encode the editing enzyme.

89. The method, use, nucleic acid, pharmaceutical composition, or kit of claim 86 or claim 88, wherein the therapeutic payload comprises a nucleic acid encoding a globin protein, wherein the globin protein comprises a γ-globin, a β-globin, and/or an α-globin.

90. The method, use, nucleic acid, pharmaceutical composition, or kit of claim 86 or claim 88, wherein the therapeutic payload comprises a nucleic acid encoding a chimeric antigen receptor (CAR), engineered T-cell receptor (TCR), checkpoint inhibitor, and/or therapeutic antibody.

91. The method, use, nucleic acid, pharmaceutical composition, or kit of any one of claims 14-90, wherein the endogenous MGMT-encoding nucleic acid is an MGMT gene in the genomes of the one or more cells.

92. The method, use, nucleic acid, pharmaceutical composition, or kit of any one of claims 14-91, wherein the endogenous MGMT-encoding nucleic acid is an MGMT mRNA transcript expressed from an MGMT gene of a genome of the one or more cells.

93. The method, use, nucleic acid, pharmaceutical composition, or kit of any one of claims 14-92, wherein the cells are hematopoietic cells.

94. The method, use, nucleic acid, pharmaceutical composition, or kit of any one of claims 14-92, wherein the cells are hematopoietic stem cells.

95. The O⁶BG-resistant MGMT polypeptide, nucleic acid, pharmaceutical composition, method, use, or kit of any one of claims 1-94, wherein the O⁶BG-resistant MGMT polypeptide has at least 80% sequence identity with SEQ ID NO: 1.
